(19) United States
(12) Patent Application Publication
(10) Pub. No.: US 2026/0166039 A1
(43) Pub. Date: Jun. 18, 2026

(54) TRICYCLIC COMPOUNDS

(71) Applicant: SCHRÖDINGER, INC., New York, NY (US)

(72) Inventors: Pieter Harm BOS, New York, NY (US); Anthony John CLARK, New York, NY (US); Aleksey Igorevich GERASYUTO, New York, NY (US); Phani GHANAKOTA, New York, NY (US); Jennifer Lynn KNIGHT, New York, NY (US); Andrew PLACZEK, New York, NY (US); Jiashi WANG, New York, NY (US)

(21) Appl. No.: 19/129,372

(22) PCT Filed: Nov. 13, 2023
(86) PCT No.: PCT/US2023/037176

§ 371 Date: May 13, 2025

Related U.S. Application Data
(60) Provisional application No. 63/425,218, filed on Nov. 14, 2022.

(51) Int. Cl. A61K 31/519; A61K 31/5377; A61K 31/5386; A61K 31/541; A61K 31/551; A61K 31/553; A61K 31/675; C07B 59/00; C07D 491/147; C07D 519/00; C07F 9/6561

(52) U.S. Cl. CPC A61K 31/519; A61K 31/5377; A61K 31/5386; A61K 31/541; A61K 31/551; A61K 31/553; A61K 31/675; C07B 59/002; C07D 491/147; C07D 519/00; C07F 9/6561; C07B 2200/05

(57) ABSTRACT

The present application relates to compounds of Formula (I), as defined herein, and pharmaceutically acceptable salts thereof. The present application also describes pharmaceutical composition comprising a compound of Formula (I), and pharmaceutically acceptable salts thereof, and methods of using the compounds and compositions for treating diseases such as cancer.

### CROSS-REFERENCE TO RELATED APPLICATIONS

This present application claims the benefit from U.S. Provisional Application No. 63/425,218, filed Nov. 14, 2022, which is hereby incorporated by reference in its entirety.

### TECHNICAL FIELD

This present application relates to fused heterocyclic compounds that are useful for treating proliferative disorders such as cancer.

### BACKGROUND

Wee1 is a highly conserved serine/threonine kinase that inhibits cell cycle progress and cell entry into mitosis through inhibitory phosphorylation of cyclin-dependent kinase 1 and 2 (CDK1 and 2). It is a key regulator of cell cycle progression through S-phase and at the G2-M checkpoint. See, e.g., Hamer, et al., Clin. Cancer Res., Vol. 17, No. 13, pp. 4200-4207 (2011) and McGowan and Russell, EMBO J., Vol. 14, No. 10, pp. 2166-2175 (1995).

In normal cells, DNA damage response (DDR) is mediated by various checkpoints which either activate the DNA repair system or induce cellular apoptosis/senescence, therefore maintaining overall genomic integrity. In cancer cells, however, with a loss of or defect in DDR due to oncogenic activation or tumor suppressor inactivation, DNA replication may persist to meet the demands of unrestrained proliferation despite the presence of unrepaired DNA lesions, which then leads to replication stress—a hallmark of cancer cells that typically includes the perturbation of error-free DNA replication and/or slow-down of DNA synthesis. See, e.g., Zhang et al, Genes, 2016, 7, 51; 1-16.

Overexpression and activation of oncogenes are a major driver of replication stress. For example, oncogenes KRAS, MYC, and CCNE1, and CDC25A result in replication stress, for example, through the creation of conflicts between replication and transcription, increasing topological stress, and/or producing a nucleotide shortage. Replication stress can cause cells to slow down replication cycles; therefore, in order to maintain its proliferative program, a cancer cell typically has ways of dealing with and resolving replication stress in order to continue growing. One example is by bypassing mechanisms of DNA damage repair, for example the loss of p53, the mutation of ATM, and defects in the homologous recombination repair pathway (such as via mutation to BRCA1, BRCA2, and PALB2). See Forment and O'Connor, Pharmacology & Therapeutics, 188 (2018) 155-167. Together, these compensatory mechanisms can result in increased genomic instability, which in turn lead to further replication stress. In general, in tumors where DNA damage response elements are bypassed or impaired, the cancer cells may become more dependent on the remaining active components of the DNA damage response and cell cycle checkpoints such as Wee1.

Inhibition of Wee1 kinase activity enhances CDK activity, and cells in S phase can be induced to enter mitosis prematurely even if DNA replication is defective or incomplete. The increased CDK activity driven by Wee1 inhibition can also rapidly increase replication initiation, leading to a shortage of nucleotides that are required for DNA replication. Wee1 inhibitors can thus be effective to enhance replicative stress and drive cancer cells undergoing a high level of this stress into premature mitosis and subsequent death from mitotic catastrophe. However, currently there are no marketed therapeutic Wee1 inhibitors.

### SUMMARY

Accordingly, provided herein is are compounds of Formula (I):

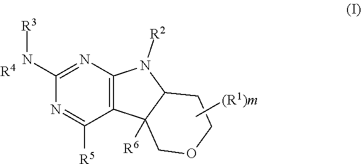

or a pharmaceutically acceptable salt thereof, wherein (R¹)ₘ, R², R³, R⁴, R⁵, R⁶, R^{A}, R^{B}, R^{C}, R^{D}, R^{E}, R^{F}, R^{G}, R^{H}, R^{I}, m, and n, are as defined herein.

Also provided herein is a pharmaceutical composition comprising a compound of Formula (I), or a pharmaceutically acceptable salt thereof, and at least one pharmaceutically acceptable excipient.

Also provided herein is a method of inhibiting mammalian cell proliferation, in vitro or in vivo, comprising contacting the mammalian cell with an effective amount of a compound of Formula (I) or a pharmaceutically acceptable salt thereof, or a pharmaceutical composition thereof as defined herein.

Also provided herein is a method of inhibiting Wee1 kinase activity in a mammalian cell, in vitro or in vivo, comprising contacting the mammalian cell with an effective amount of a compound of Formula (I) or a pharmaceutically acceptable salt thereof, or a pharmaceutical composition thereof as defined herein.

Also provided herein is a method of treating cancer, for example, uterine, ovarian, breast, gastric, colorectal, and non-small cell lung cancer, in a subject in need thereof, comprising administering to the subject an effective amount of a compound of Formula (I), or a pharmaceutically acceptable salt thereof, or a pharmaceutical composition thereof as defined herein.

Also provided herein is a method of treating a cancer in a subject in need thereof, the method comprising:
(a) identifying the cancer as having replication stress; and
(b) administering to the subject an effective amount of a compound of Formula (I), or a pharmaceutically acceptable salt thereof, or a pharmaceutical composition thereof as defined herein.

Also provided herein is a method of treating a cancer in a subject in need thereof, the method comprising:
administering an effective amount of a compound of Formula (I), or a pharmaceutically acceptable salt thereof, or a pharmaceutical composition thereof as defined herein, to a subject identified as having a cancer having replication stress.

Also provided herein is a method of treating a cancer in a subject in need thereof, the method comprising:
(a) identifying the cancer as having an inactivated tumor suppressor gene; and
(b) administering to the subject an effective amount of a compound of Formula (I), or a pharmaceutically acceptable salt thereof, or a pharmaceutical composition thereof as defined herein.

Also provided herein is a method of treating a cancer in a subject in need thereof, the method comprising:
administering an effective amount of a compound of Formula (I), or a pharmaceutically acceptable salt thereof, or a pharmaceutical composition thereof as defined herein, to a subject identified as having a cancer having an inactivated tumor suppressor gene.

Also provided herein is a method of treating a cancer in a subject in need thereof, the method comprising:
(a) identifying the cancer as having an activated oncogene; and
(b) administering to the subject an effective amount of a compound of Formula (I), or a pharmaceutically acceptable salt thereof, or a pharmaceutical composition thereof as defined herein.

Also provided herein is a method of treating a cancer in a subject in need thereof, the method comprising:
administering an effective amount of a compound of Formula (I), or a pharmaceutically acceptable salt thereof, or a pharmaceutical composition thereof as defined herein, to a subject identified as having a cancer having an activated oncogene.

Also provided herein is a method of treating a cancer in a subject in need thereof, the method comprising:
(i) administering to the subject an effective amount of a therapy comprising:
(a) a DNA-damaging agent;
(b) a DNA repair inhibiting agent;
(c) radiation;
(d) (a) and (b);
(e) (a) and (c);
(f) (b) and (c);
(g) (a), (b), and (c); and
(ii) after (i), administering to the subject an effective amount of a compound of Formula (I), or a pharmaceutically acceptable salt thereof, or a pharmaceutical composition thereof as defined herein.

Also provided herein is a method of treating a cancer in a subject in need thereof, the method comprising:
administering an effective amount of a compound of Formula (I), or a pharmaceutically acceptable salt thereof, or a pharmaceutical composition thereof as defined herein, to a subject previously administered one or more doses of a therapy comprising:
(a) a DNA-damaging agent;
(b) a DNA repair inhibiting agent;
(c) radiation;
(d) (a) and (b);
(e) (a) and (c);
(f) (b) and (c);
(g) (a), (b), and (c).

Also provided herein is a method of treating a cancer in a subject in need thereof, the method comprising:
administering to the subject:
(i) an effective amount of a compound of Formula (I), or a pharmaceutically acceptable salt thereof, or a pharmaceutical composition thereof as defined herein; and
(ii) an effective amount of a therapy comprising:
(a) a DNA-damaging agent;
(b) a DNA repair inhibiting agent;
(c) radiation;
(d) (a) and (b);
(e) (a) and (c);
(f) (b) and (c);
(g) (a), (b), and (c).

Also provided herein is a method for inducing mitotic collapse in a mammalian cell, comprising contacting the mammalian cell with a compound of Formula (I), or a pharmaceutically acceptable salt thereof.

Also provided herein is a compound of Formula (I), or a pharmaceutically acceptable salt thereof or a pharmaceutical composition thereof as defined herein, for use in the treatment of cancer (e.g., a cancer with replication stress).

Also provided herein is a compound of Formula (I), or a pharmaceutically acceptable salt thereof, or a pharmaceutical composition thereof as defined herein for use in the treatment of cancer.

Also provided herein is a compound of Formula (I), or a pharmaceutically acceptable salt thereof for use in the inhibition of Wee1 kinase activity.

Also provided herein is a compound of Formula (I), or a pharmaceutically acceptable salt thereof, as defined herein, in the manufacture of a medicament for the treatment of cancer (e.g., a cancer with replication stress). In some embodiments, the cancer is selected from one or more of uterine, ovarian, breast, gastric, colorectal, and non-small cell lung.

Also provided herein is a compound of Formula (I), or a pharmaceutically acceptable salt thereof, defined herein in the manufacture of a medicament for the inhibition of Wee1 kinase activity.

Also provided herein is a process for preparing a compound of Formula (I), or a pharmaceutically acceptable salt thereof.

Also provided herein is a compound of Formula (I), or a pharmaceutically acceptable salt thereof obtained by a process of preparing the compound as defined herein.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the disclosure will be apparent from the following detailed description and figures, and from the claims.

### DETAILED DESCRIPTION

### Definitions

The term “compound,” as used herein is meant to include all stereoisomers, geometric isomers, tautomers, and isotopically enriched variants of the structures depicted. Compounds herein identified by name or structure as one particular tautomeric form are intended to include other tautomeric forms unless otherwise specified.

The term “tautomer,” as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the disclosure, and the naming of the compounds does not exclude any tautomer. An example of a tautomeric forms includes the following example:

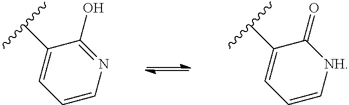 [SMILES: *c1cccnc1O ; *c1ccc[nH]c1=O]

It will be appreciated that certain compounds provided herein may contain one or more centers of asymmetry and may therefore be prepared and isolated in a mixture of isomers such as a racemic mixture, or in an enantiomerically pure form.

The term “halo” refers to one of the halogens, group 17 of the periodic table. In particular the term refers to fluorine, chlorine, bromine and iodine. Preferably, the term refers to fluorine or chlorine.

The term “C1-C6 alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl, n-pentyl and n-hexyl. Similarly, a C1-C3 alkyl group is a linear or branched hydrocarbon chain containing 1, 2, or 3 carbon atoms.

The term “C1-C6 deuteroalkyl” refers to an alkyl group, as described herein, where one or more hydrogen atoms are replaced with deuterium, such as —CD₃.

The term “C1-C6 alkoxy” refers to a C1-C6 alkyl group which is attached to a molecule via an oxygen atom. This includes moieties where the alkyl part may be linear or branched, such as methoxy, ethoxy, n-propoxy, iso-propoxy, n-butoxy, sec-butoxy, tert-butoxy, n-pentoxy and n-hexoxy.

As used herein, the term “cyano” refers to a —CN radical.

As used herein, the term “hydroxyl” refers to an —OH radical.

As used herein, the term “amino” refers to a —NH₂ radical.

As used herein, the term “C6-C10 aryl” refers to a 6 to 10 carbon mono- or bicyclic ring system wherein at least one ring in the system is aromatic. Non-limiting examples of aryl groups include phenyl, naphthyl, tetrahydronaphthyl. In bicyclic ring systems where only one ring is aromatic, the non-aromatic ring can be a cycloalkyl group, as defined herein.

As used herein, the term “heteroaryl” refers to a mono- or bicyclic ring system with, for example, 5 to 10 ring atoms, wherein the ring system is aromatic; wherein one or more carbon atoms in at least one ring in the system is/are replaced with an heteroatom independently selected from N, O, and S. Non-limiting examples of heteroaryl groups include pyridine, pyrimidine, pyrrole, pyrazole, imidazole, and indole.

As used herein, the term “cycloalkyl” refers to a saturated or partially unsaturated 3-10 mono- or bicyclic hydrocarbon group; wherein bicyclic systems include fused, spiro (optionally referred to as “spirocycloalkyl” groups), and bridged ring systems. Non-limiting examples of cycloalkyl groups include cyclopropyl, cyclohexyl, spiro[2.3]hexyl, and bicyclo[1.1.1]pentyl.

The term “heterocyclyl” refers to a saturated or partially unsaturated 3-12 membered hydrocarbon monocyclic or bicyclic ring system, having at least one heteroatom within the ring selected from N, O and S. Bicyclic heterocyclyl groups include fused, spiro, and bridged ring systems. The heterocyclyl ring system may include oxo substitution at one or more C, N, or S ring members. In bicyclic ring systems, one ring can be aromatic, if the other ring is not aromatic. For example, one ring could be phenyl and the other ring could be pyrrolidine, or, one ring could be pyridine and the other ring could be cyclohexane. The heterocyclyl group may be denoted as, for example, a “5-10 membered heterocyclyl group,” which is a ring system containing 5, 6, 7, 8, 9 or 10 atoms at least one being a heteroatom. For example, there may be 1, 2 or 3 heteroatoms, optionally 1 or 2. The heterocyclyl group may be bonded to the rest of the molecule through any carbon atom or through a heteroatom such as nitrogen. Exemplary heterocyclyl groups include, but are not limited to, piperidinyl, piperazinyl, morpholino, tetrahydropyranyl, azetidinyl, oxetanyl, 2-azaspiro[3.3]heptanyl, pyrrolidin-2-one, sulfolane, isothiazoline S,S-dioxide, and decahydronaphthalenyl.

The term “heterocyclyloxy” refers to a heterocyclyl group which is attached to a molecule via an oxygen atom.

As used herein, the term “oxo” refers to an “═O” group attached to a carbon atom.

As used herein, the symbol [symbol] depicts the point of attachment of an atom or moiety to the indicated atom or group in the remainder of the molecule.

The compounds of Formula (I) include pharmaceutically acceptable salts thereof. In addition, the compounds of Formula (I) also include other salts of such compounds which are not necessarily pharmaceutically acceptable salts, and which may be useful as intermediates for preparing and/or purifying compounds of Formula (I) and/or for separating enantiomers of compounds of Formula (I). Non-limiting examples of pharmaceutically acceptable salts of compounds of Formula (I) include trifluoroacetic acid and hydrochloride salts.

It will further be appreciated that the compounds of Formula (I) or their salts may be isolated in the form of solvates, and accordingly that any such solvate is included within the scope of the present disclosure. For example, compounds of Formula (I) and salts thereof can exist in unsolvated as well as solvated forms with pharmaceutically acceptable solvents such as water, ethanol, and the like.

In one embodiment, the compounds of Formula (I) include the compounds of Examples 1-832 and stereoisomers and pharmaceutically acceptable salts thereof. In some embodiments, the compounds of Examples 1-832 are present in the form of a free base. In some embodiments, the compounds of Examples 1-832 are present in the form of a pharmaceutically acceptable salt.

The term “pharmaceutically acceptable” indicates that the compound, or salt or composition thereof is compatible chemically and/or toxicologically with the other ingredients comprising a formulation and/or the subject being treated therewith.

Compounds provided herein may also contain unnatural proportions of atomic isotopes at one or more of the atoms that constitute such compounds. That is, an atom, in particular when mentioned in relation to a compound according to Formula (I), comprises all isotopes and isotopic mixtures of that atom, either naturally occurring or synthetically produced, either with natural abundance or in an isotopically enriched form. For example, when hydrogen is mentioned, it is understood to refer to ¹H, ²H, ³H or mixtures thereof; when carbon is mentioned, it is understood to refer to ¹¹C, ¹²C, ¹³C, ¹⁴C or mixtures thereof; when nitrogen is mentioned, it is understood to refer to ¹³N, ¹⁴N, ¹⁵N or mixtures thereof; when oxygen is mentioned, it is understood to refer to ¹⁴O, ¹⁵O, ¹⁶O, ¹⁷O, ¹⁸O or mixtures thereof; and when fluoro is mentioned, it is understood to refer to ¹⁸F, ¹⁹F or mixtures thereof; unless expressly noted otherwise. For example, in deuteroalkyl and deuteroalkoxy groups, where one or more hydrogen atoms are specifically replaced with deuterium (²H). As some of the aforementioned isotopes are radioactive, the compounds provided herein therefore also comprise compounds with one or more isotopes of one or more atoms, and mixtures thereof, including radioactive compounds, wherein one or more non-radioactive atoms has been replaced by one of its radioactive enriched isotopes. Radiolabeled compounds are useful as therapeutic agents, e.g., cancer therapeutic agents, research reagents, e.g., assay reagents, and diagnostic agents, e.g., in vivo imaging agents. All isotopic variations of the compounds provided herein, whether radioactive or not, are intended to be encompassed within the scope of the present disclosure.

Protecting groups can be a temporary substituent which protects a potentially reactive functional group from undesired chemical transformations. The choice of the particular protecting group employed is well within the skill of one of ordinary skill in the art. A number of considerations can determine the choice of protecting group including, but not limited to, the functional group being protected, other functionality present in the molecule, reaction conditions at each step of the synthetic sequence, other protecting groups present in the molecule, functional group tolerance to conditions required to remove the protecting group, and reaction conditions for the thermal decomposition of the compounds provided herein. The field of protecting group chemistry has been reviewed in Greene, T. W.; Wuts, P. G. M. Protective Groups in Organic Synthesis, 2^{nd} ed.; Wiley: New York, 1991, which is incorporated by reference herein in its entirety.

The ability of selected compounds to act as Wee1 inhibitors may be demonstrated by the biological assays described herein. IC₅₀ values are shown in Table A.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

As used herein, the term “subject” refers to any animal, including mammals such as humans. In some embodiments, the subject is a human. In some embodiments, the subject has experienced and/or exhibited at least one symptom of the disease or disorder to be treated and/or prevented.

Persistent replication stress (sometimes also called replicative stress) is a phenomenon that is observed in cancer cells and is rarely observed in non-cancerous cells. One hallmark of replication stress is fork stalling. In some embodiments, a tumor that has “replication stress” is one that has stalled replication forks. In many cases, when DNA damage occurs to a strand being replicated, the replication machinery cannot pass the lesion, resulting in fork stalling. To repair the stalled replication fork, single-stranded DNA (ssDNA) on the leading strand is typically exposed, initiating the Replication protein A (RPA) to bind to the ssDNA and activate the ATR/Chk1 pathway. By activating this pathway, entry into M phase is limited. If replication stress is exacerbated, for example, by inactivation of one or more tumor suppressor genes (e.g., p53, RB1, CDKN2A, BRCA1, BRCA2, FBXW7, SETD2, NOTCH1 or a combination thereof) (for instance, resulting in the premature onset of S phase), activation of one or more oncogenes (e.g., Cyclin E, CDC25A, Myc, a RAS gene (e.g., KRAS, NRAS, HRAS, or a combination thereof), or a combination thereof), increased DNA damage (e.g., through reactive oxygen species (ROS), chemotherapy (e.g., platinum-based chemotherapy, alkylating agents, nucleobase/nucleoside/nucleotide analogs, topoisomerase I and/or II inhibitors, PARP1 and/or PARP2 inhibitors, ATR inhibitors, Chk1 inhibitors), and/or radiation therapy), premature entry into M phase (e.g., via inhibition of Wee1), or a combination thereof, mitotic catastrophe can occur, leading to cell death. See, e.g., U.S. Publication No. 2020/0157638, Zhang et al, Genes, 2016, 7, 51; 1-16, Berti and Vindigni Nature Structural & Molecular Biology, 2016, 23, 2: 103-109, and Ren et al. Oncotarget, 2017 8, 23: 36996. Without being bound by any particular theory, it is believed that cells that have replication stress are more dependent on the activity of Wee1 (e.g., to prevent aberrant entry into M phase) due to the dysregulation of one or more other mechanisms that typically regulate the cell cycle.

In some embodiments, the subject has been identified or diagnosed as having a cancer with replication stress. In some embodiments, the subject has a tumor that is positive for replication stress. The subject can be a subject with a tumor(s) that tests positive for replication stress. The subject can be a subject whose tumors have replication stress. In some embodiments, the subject is suspected of having a tumor with replication stress. In some embodiments, the subject has a clinical record indicating that the subject has a tumor that has replication stress. In some embodiments, the subject is a pediatric subject. In some embodiments, the subject has been identified or diagnosed as having a cancer that, based on histological examination, is determined to be associated with replication stress. The presence of replication stress in a subject (e.g., in a tumor of a subject (e.g., a sample of the tumor)) can be detected in any appropriate way. In some embodiments, detection of replication stress can be detected directly. In some embodiments, replication stress can be detected indirectly. In some embodiments, replication stress can be detected using H2AX immunohistological staining to measure, for example, γH2AX. In some embodiments, replication stress can be detected by measuring cleaved caspase. In some embodiments, replication stress can be detected using a terminal deoxynucleotidyl transferase-mediated deoxyuridine triphosphate nick-end labeling (TUNEL) assay. In some embodiments, replication stress can be detected by measuring the immune response to cytosolic DNA. See, e.g., Ubhi and Brown. Cancer Research 79.8 (2019): 1730-1739.

In some embodiments, replication stress can be detected via DNA fiber analyses, for example, by measuring DNA synthesis rates of individual DNA replication forks. In some embodiments, replication stress can be detected via DNA pull-downs to identify proteins bound directly at replication forks in vivo. See, e.g., Ubhi and Brown. Cancer Research 79.8 (2019): 1730-1739.

In some embodiments, replication stress can be detected using a biomarker of replication stress. In some embodiments, a biomarker of replication stress can include Ki-67, Cyclin E, POLD3, γH2AX, FANCD2, or a combination thereof. In some embodiments, a biomarker of replication stress can include pH2AX Ser139 (γH2AX), pATR Thr1989, pCHK1 Ser345, pRPA32 Ser33, or a combination thereof. See, e.g., Forment and O'Connor, Pharmacology & Therapeutics, 188 (2018) 155-16. In some embodiments, a biomarker of replication stress can be an activated oncogene. In some embodiments, a biomarker of replication stress can be an inactivated tumor suppressor gene. In some embodiments, a biomarker of replication stress can be one or more genes listed in Tables 1A or 1B in WO 2019/173456 (A1). In some embodiments, two or more of these methods can be combined. For example, in some embodiments, replication stress can be detected using the p53 status of the tumor(s) of the subject, optionally combined with the proliferation index of the tumor(s) (e.g., as measured by Ki67). See, e.g., Reaper et al. Nature Chemical Biology 7.7 (2011): 428-430. In some embodiments, replication stress can be detected using chromosomal instability (e.g., by karyotype or by measuring chromosomal instability genes). See, e.g., Burrell et al. Nature 494.7438 (2013): 492-496.

In some embodiments, the subject has been identified or diagnosed as having a cancer with an inactivation of one or more tumor suppressor genes (e.g., as determined using a regulatory agency-approved, e.g., FDA-approved, assay or kit). In some embodiments, the subject has a tumor that is positive for inactivation of one or more tumor suppressor genes (e.g., as determined using a regulatory agency-approved, e.g., FDA-approved, assay or kit). The subject can be a subject with a tumor(s) that is positive for inactivation of one or more tumor suppressor genes (e.g., identified as positive using a regulatory agency-approved, e.g., FDA-approved, assay or kit). The subject can be a subject whose tumors have inactivation of one or more tumor suppressor genes (e.g., where the tumor is identified as such using a regulatory agency-approved, e.g., FDA-approved, kit or assay). In some embodiments, the subject is suspected of having a cancer with inactivation of one or more tumor suppressor genes. In some embodiments, the subject has a clinical record indicating that the subject has a tumor that has inactivation of one or more tumor suppressor genes (and optionally the clinical record indicates that the subject should be treated with any of the compositions provided herein). In some embodiments, the subject is a pediatric subject. In some embodiments, the subject has been identified or diagnosed as having a cancer that, based on histological examination, is determined to inactivation of one or more tumor suppressor genes. Inactivation of a tumor suppressor gene can be through any appropriate mechanism, including, but not limited to, gene deletion, inactivating mutation, inactivating translocation, transcriptional silencing, epigenetic alteration, and degradation of mRNA and/or protein products of the gene.

A tumor suppressor gene can be any appropriate tumor suppressor gene. In some embodiments, a tumor suppressor gene can be p53, RB1, CDKN2A, BRCA1, BRCA2, FBXW7, SETD2, NOTCH1, or a combination thereof. See, e.g., Forment and O'Connor, Pharmacology & Therapeutics, 188 (2018) 155-167, Reaper et al. Nature Chemical Biology 7.7 (2011): 428-430, and Méndez et al. Clinical Cancer Research 24.12 (2018): 2740-2748. In some embodiments, an inactivated tumor suppressor gene is a mutated p53 gene. In some embodiments, an inactivated tumor suppressor gene is a deleted p53 gene. In some embodiments, an inactivated tumor suppressor gene is a mutated CDKN2A gene. In some embodiments, an inactivated tumor suppressor gene is a mutated NOTCH1 gene. In some embodiments, an inactivated tumor suppressor gene is a deleted FBXW7 gene. A non-limiting example of a cancer that can have a deleted FBXW7 gene is uterine serous carcinoma. In some embodiments, an inactivated tumor suppressor gene is a mutated FBXW7 gene. In some embodiments, an inactivated tumor suppressor gene is a mutated RB1 gene. In some embodiments, an inactivated tumor suppressor gene is a deleted BRCA1 gene. In some embodiments, an inactivated tumor suppressor gene is a mutated BRCA1 gene. In some embodiments, an inactivated tumor suppressor gene is a BRCA1 gene with a hypermethylated promoter region. In some embodiments, an inactivated tumor suppressor gene is a deleted BRCA2 gene. In some embodiments, an inactivated tumor suppressor gene is a mutated BRCA2 gene. In some embodiments, an inactivated tumor suppressor gene is a BRCA2 gene with a hypermethylated promoter region. In some embodiments, an inactivated tumor suppressor gene is a mutated NOTCH1 gene. In some embodiments, an inactivated tumor suppressor gene is a mutated SETD2 gene.

In some embodiments, the subject has been identified or diagnosed as having a cancer with an activation of one or more oncogenes (e.g., as determined using a regulatory agency-approved, e.g., FDA-approved, assay or kit). In some embodiments, the subject has a tumor that is positive for activation of one or more oncogenes (e.g., as determined using a regulatory agency-approved, e.g., FDA-approved, assay or kit). The subject can be a subject with a tumor(s) that is positive for activation of one or more oncogenes (e.g., identified as positive using a regulatory agency-approved, e.g., FDA-approved, assay or kit). The subject can be a subject whose tumors have activation of one or more oncogenes (e.g., where the tumor is identified as such using a regulatory agency-approved, e.g., FDA-approved, kit or assay). In some embodiments, the subject is suspected of having a cancer with activation of one or more oncogenes. In some embodiments, the subject has a clinical record indicating that the subject has a tumor that has activation of one or more oncogenes (and optionally the clinical record indicates that the subject should be treated with any of the compositions provided herein). In some embodiments, the subject is a pediatric subject. In some embodiments, the subject has been identified or diagnosed as having a cancer that, based on histological examination, is determined to activation of one or more oncogenes. Activation of an oncogene can be through any appropriate mechanism, including, but not limited to, gene amplification, activating mutation, activating translocation, transcriptional activation, epigenetic alteration, and/or overexpression of the protein product of the oncogene.

An oncogene can be any appropriate oncogene. In some embodiments, an oncogene can be cyclin E (sometimes also called cyclin E1 or CCNE1), CDC25A, Myc, a RAS gene (e.g., KRAS, NRAS, HRAS, or a combination thereof), or a combination thereof. See, e.g., Haigis, Trends in Cancer 3.10 (2017): 686-697, Kalkat, et al. Genes (2017) 8, 151, Feng et al. Molecular and Cellular Biology 31.16 (2011): 3457-3471, Kok et al. Oncogenesis (2020) 9:88, Dang, Cell 149.1 (2012): 22-35. In some embodiments, an activated oncogene is an amplified cyclin E gene. Non-limiting examples of cancers that can have amplified cyclin E (e.g., cyclin E1) include rhabdomyosarcoma, urinary bladder adenocarcinoma, malignant fibrous histiocytoma, small intestine adenocarcinoma, medullary breast cancer, gallbladder adenocarcinoma, stomach adenocarcinoma, urinary bladder transitional cell carcinoma, urinary bladder small cell carcinoma, non-serous ovarian carcinoma, uterine cervix squamous cell carcinoma, and ovarian endometrial (endometrioid) carcinoma. In some embodiments, an activated oncogene is an overexpressed CDC25A. Non-limiting examples of cancer that can have overexpressed CDC25A include breast cancer, colorectal cancer, lung cancer, hepatocellular carcinoma, prostate cancer, esophageal cancer (e.g., esophageal squamous cell carcinoma), pancreatic ductal adenocarcinoma, thyroid neoplasms, non-Hodgkin's lymphoma, and neuroblastoma. In some embodiments, an activated oncogene is an amplified Myc gene. Non-limiting examples of cancers that can have Myc amplification include breast invasive ductal carcinoma, lung adenocarcinoma, prostate adenocarcinoma, colon adenocarcinoma, and high grade ovarian serous adenocarcinoma. In some embodiments, an activated oncogene is a Myc gene with an activating translocation. In some embodiments, an activated oncogene is a transcriptionally activated Myc gene. In some embodiments, an activated oncogene is a mutated RAS gene (e.g., a KRAS gene, an NRAS gene, an HRAS gene, or a combination thereof). In some embodiments, a mutated RAS gene (e.g., a KRAS gene, an NRAS gene, an HRAS gene, or a combination thereof) includes a mutation at position G12 of the protein product of the gene. In some embodiments, a mutated RAS gene (e.g., a KRAS gene, an NRAS gene, an HRAS gene, or a combination thereof) includes a mutation at position G13 of the protein product of the gene. In some embodiments, a mutated RAS gene (e.g., a KRAS gene, an NRAS gene, an HRAS gene, or a combination thereof) includes a mutation at position Q61 of the protein product of the gene. Non-limiting examples of cancers that can have KRAS mutations include pancreatic ductal adenocarcinoma (PDAC), colorectal cancer (CRC), and non-small cell lung cancer (NSCLC).

In some embodiments, the subject has been identified or diagnosed as having a cancer with increased DNA damage. In some embodiments, the subject has a tumor that is positive for increased DNA damage. The subject can be a subject with a tumor(s) that tests positive for increased DNA damage. The subject can be a subject whose tumors have increased DNA damage. In some embodiments, the subject is suspected of having a tumor with increased DNA damage. In some embodiments, the subject has a clinical record indicating that the subject has a tumor that has increased DNA damage. In some embodiments, the subject is a pediatric subject. In some embodiments, the subject has been identified or diagnosed as having a cancer that, based on histological examination, is determined to be associated with increased DNA damage.

Typically, “increased” DNA damage is achieved by administration of one or more DNA-damaging agents, one or more DNA repair inhibiting agents, and/or radiation to the subject. In some embodiments, a DNA-damaging agent can include a platinum-based chemotherapy, an alkylating agent, a nucleobase, nucleoside, and/or nucleotide analog, or a combination thereof. In some embodiments, a DNA repair inhibiting agent can include a topoisomerase I inhibitor, a topoisomerase II inhibitor, a PARP inhibitor, an ATR inhibitor, a Chk inhibitor, or a combination thereof. Non-limiting examples of platinum-based chemotherapeutics include carboplatin, cisplatin, and oxaplatin. Non-limiting examples of alkylating agents include cyclophosphamide, carmustine, busulfan, procarbazine, dacarbazine, temozoloamide, thiotepa, and mitomycin C. Non-limiting examples of nucleobase, nucleoside, and/or nucleotide analogs include fluorouracil, cytarabine, gemcitabine, azacitidine, and decitabine. Non-limiting examples of topoisomerase I inhibitors include topotecan, irinotecan, belotecan, and camptothecin. Non-limiting examples of topoisomerase II inhibitors include etoposide, tenoposide, doxorubicin, daunorubicin, epirubicin, and idarubacin. Non-limiting examples of PARP inhibitors include olaparib, niraparib, rucaparib, talazoparib, and veliparib. Non-limiting examples of ATR inhibitors include AZD6738, BAY1895344, and M6620. Non-limiting examples of Chk1 inhibitors include prexasertib, GDC-0575, SCH 900776, and SRA737.

The term “pediatric subject” as used herein refers to a subject under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

In certain embodiments, compounds of Formula (I), or a pharmaceutically acceptable salt thereof are useful for preventing diseases and disorders as defined herein (for example, cancer). The term “preventing” as used herein means the prevention of the onset, recurrence or spread, in whole or in part, of the disease or condition as described herein, or a symptom thereof.

Without being bound by any particular theory, it is believed that cancers that exhibit replication stress are more reliant on the cell cycle checkpoint regulators such as Wee1. In some embodiments, cancers that exhibit replication stress overexpress Wee1. Non-limiting examples of cancers that can overexpress Wee1 include hepatocellular carcinoma, breast cancers, cervical cancers, lung cancers, squamous cell carcinoma, diffuse intrinsic pontine glioma, glioblastoma, medulloblastoma, leukemia, melanoma, ovarian cancers, pancreatic cancers, and colorectal cancers. See, e.g., P Reigan et al Trends in Pharmacol Sci 2016; Mir, et al., Cancer Cell, Vol. 18, No. 3, pp. 244-257 (2010)).

The term “regulatory agency” refers to a country's agency for the approval of the medical use of pharmaceutical agents with the country. For example, a non-limiting example of a regulatory agency is the U.S. Food and Drug Administration (FDA).

### Compounds

Accordingly, provided herein are compounds of Formula (I):

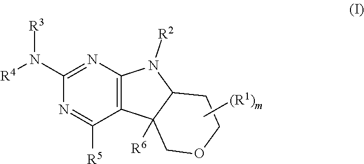

or a pharmaceutically acceptable salt thereof, wherein:
each R¹ is independently C1-C6 alkyl;
m is 0, 1, or 2;
R² is hydrogen, C1-C6 alkyl, phenyl, 5-10 membered heteroaryl, or 5-10 membered heterocyclyl, wherein the phenyl, 5-10 membered heteroaryl, and 5-10 membered heterocyclyl are each optionally substituted with 1-3 substituents each independently selected from R^{A};
each R^{A} is independently selected from: halogen, cyano, —NR^{B}R^{C}, —C(═O)NR^{B}R^{C}, —N═S(O)Me)₂, C1-C6 alkyl optionally substituted with hydroxyl or —NR^{B}R^{C}; C3-C6 cycloalkyl optionally substituted with —NR^{B}R^{C}; and 4-6 membered heterocyclyl optionally substituted with 1-2 substituents independently selected from halogen and C1-C6 alkyl;
each R^{B} and R^{C} are independently hydrogen or C1-C6 alkyl;
R³ is hydrogen or C1-C6 alkyl;
R⁴ is
(i) phenyl optionally substituted with 1 or 2 substituents each independently selected from the group consisting of: halogen, cyano, —SO₂(C1-C6 alkyl), C1-C6 haloalkyl, C1-C6 deuteroalkyl, C1-C6 alkyl optionally substituted with 1 or 2 substituents each independently selected from: —NR^{B}R^{C} and —CO₂H; —(C1-C6 alkyl)ₙ-C(═O)NR^{E}R^{F}, C3-C6 cycloalkyl optionally substituted with C1-C6 alkyl; 5-6 membered heteroaryl optionally substituted with C1-C6 alkyl; 4-12 membered heterocyclyloxy optionally substituted with 1 or 2 independently selected R^{G}; and 4-12 membered heterocyclyl optionally substituted with 1 or 2 independently selected R^{G};
(ii) 9-12 membered heterocyclyl optionally substituted with 1-3 independently selected C1-C6 alkyl;
(iii) 5-10 membered heteroaryl optionally substituted with 1 or 2 substituents independently selected from the group consisting of C1-C6 alkyl, C1-C6 haloalkyl, C1-C6 deuteroalkyl, C3-C6 cycloalkyl, 5-6 membered heteroaryl optionally substituted with C1-C6 alkyl; 4-12 membered heterocyclyloxy optionally substituted with 1 or 2 independently selected R^{G}; and 4-12 membered heterocyclyl optionally substituted with 1 or 2 C1-C6 alkyl or amino;
(iv) C3-C6 cycloalkyl; or
(v) C(O)—R^{I};
n is 0 or 1;
each R^{E} and R^{F} are independently hydrogen or C1-C6 alkyl; or
R^{E} and R^{F}, together with the nitrogen atom to which they are attached, form a 4-8 membered heterocyclyl optionally substituted with C1-C6 alkyl;
each R^{G} is independently halogen, C1-C6 alkyl, C1-C6 deuteroalkyl, —NR^{B}R^{C}, or ═NR^{H};
R^{H} is hydrogen or C1-C6 alkyl;
R^{I} is C1-C6 alkyl, phenyl optionally substituted with 1 to 3 halogen; 5-6 membered heteroaryl optionally substituted with 1 to 3 C1-C6 alkyl; C3-C6 cycloalkyl optionally substituted with 1 substituent selected from the group consisting of: halogen, phenyl, and 5-6 membered heteroaryl optionally substituted with 1 to 3 C1-C6 alkyl;
R⁵ is hydrogen, halogen, or C1-C6 alkyl; and
R⁶ is hydrogen or C1-C6 alkyl.

In some embodiments, m is 0. In some embodiments, m is 1. In some embodiments, m is 2.

In some embodiments, each R¹ is independently C1-C6 alkyl. In some embodiments, each R¹ is independently C1-C3 alkyl. In some embodiments, each R¹ is the same. In some embodiments, each R¹ is different. In some embodiments, each R¹ is methyl. In some embodiments, m is 2 and each R¹ is methyl. In some embodiments, two independently selected (R¹)ₘ groups are geminal. In some embodiments, m is 2 and each R¹ is methyl. In some embodiments, m is 2, each R¹ is methyl, and the methyl groups are geminal. In some embodiments, two independently selected R¹ groups are geminal, and the carbon atom to which they are attached is adjacent to the ring oxygen.

In some embodiments, R² is phenyl optionally substituted with 1-3 independently selected R^{A}. In some embodiments, R² is phenyl substituted with 1-3 independently selected R^{A}. In some embodiments, R² is phenyl substituted with 3 independently selected R^{A}. In some embodiments, R² is phenyl substituted with 1 or 2 independently selected R^{A}. In some embodiments, R² is phenyl substituted with 2 independently selected R^{A}. In some embodiments, R² is phenyl substituted with one R^{A}.

In some embodiments, R² is phenyl substituted with 1-3 independently selected R^{A}, wherein 1 or 2 R^{A} groups are ortho to the point of attachment of R² to the remainder of Formula (I). In some embodiments, R² is phenyl substituted with 1-3 independently selected R^{A}, wherein 1 or 2 R^{A} groups are meta to the point of attachment of R² to the remainder of Formula (I). In some embodiments, R² is phenyl substituted with 1-3 independently selected R^{A}, wherein one R^{A} group is para to the point of attachment of R² to the remainder of Formula (I).

In some embodiments, R² is an unsubstituted phenyl.

In some embodiments, R² is a 5-10 membered heteroaryl optionally substituted with 1-3 independently selected R^{A}. In some embodiments, R² is a 5-10 membered heteroaryl substituted with 1-3 independently selected R^{A}. In some embodiments, R² is a 5-10 membered heteroaryl substituted with 3 independently selected R^{A}. In some embodiments, R² is a 5-10 membered heteroaryl substituted with 1 or 2 independently selected R^{A}. In some embodiments, R² is a 5-10 membered heteroaryl substituted with 2 independently selected R^{A}. In some embodiments, R² is a 5-10 membered heteroaryl substituted with one R^{A}.

In some embodiments, R² is a 5-6 membered heteroaryl optionally substituted with 1-3 independently selected R^{A}. In some embodiments, R² is a 5-6 membered heteroaryl substituted with 1-3 independently selected R^{A}. In some embodiments, R² is a 5-6 membered heteroaryl substituted with 3 independently selected R^{A}. In some embodiments, R² is a 5-6 membered heteroaryl substituted with 1 or 2 independently selected R^{A}. In some embodiments, R² is a 5-6 membered heteroaryl substituted with 2 independently selected R^{A}. In some embodiments, R² is a 5-6 membered heteroaryl substituted with one R^{A}.

In some embodiments, R² is pyridyl, pyrazinyl, pyridazinyl, or pyrimidinyl optionally substituted with 1-3 independently selected R^{A}. In some embodiments, R² is pyridyl, pyrazinyl, pyridazinyl, or pyrimidinyl substituted with 1-3 independently selected R^{A}. In some embodiments, R² is pyridyl, pyrazinyl, pyridazinyl or pyrimidinyl substituted with 3 independently selected R^{A}. In some embodiments, R² is pyridyl, pyrazinyl, pyridazinyl or pyrimidinyl substituted with 2 independently selected R^{A}. In some embodiments, R² is pyridyl, pyrazinyl, pyridazinyl or pyrimidinyl substituted with one R^{A}.

In some embodiments, R² is pyridyl substituted with 1-3 independently selected R^{A}, for example, a 2-pyridyl, 3-pyridyl, or 4-pyridyl.

In some embodiments, R² is selected from the group consisting of

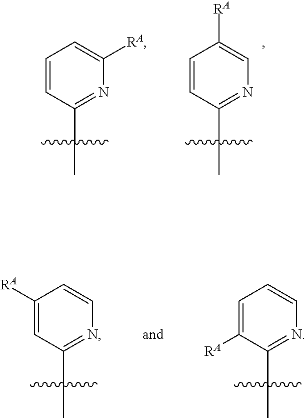

In some embodiments, R² is selected from the group consisting of

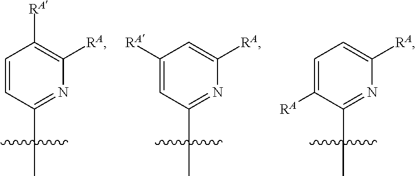

wherein R^{A′} is independently selected from R^{A}.

In some embodiments, R² is selected from the group consisting of

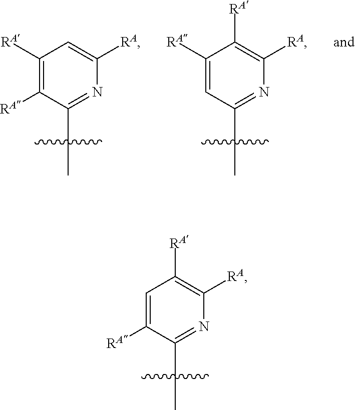

wherein R^{A′} and R^{A″} are each independently selected from R^{A}.

In some embodiments, R² is

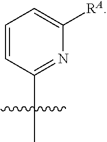

In some embodiments, R² is

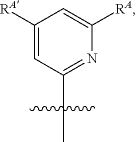

wherein R^{A′} is independently selected from R^{A}.

In some embodiments, R² is an unsubstituted 5-10 membered heteroaryl. In some embodiments, R² is an unsubstituted 5-6 membered heteroaryl. In some embodiments, R² is an unsubstituted pyridyl, pyrazinyl, pyridazinyl, or pyrimidinyl.

In some embodiments, R² is a 5-10 membered heterocyclyl optionally substituted with 1-3 independently selected R^{A}. In some embodiments, R² is a 5-10 membered heterocyclyl substituted with 1-3 independently selected R^{A}. In some embodiments, R² is a 5-10 membered heterocyclyl substituted with 3 independently selected R^{A}. In some embodiments, R² is a 5-10 membered heterocyclyl substituted with 1 or 2 independently selected R^{A}. In some embodiments, R² is a 5-10 membered heterocyclyl substituted with 2 independently selected R^{A}. In some embodiments, R² is a 5-10 membered heterocyclyl substituted with one R^{A}.

In some embodiments, R² is

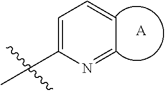

optionally substituted with 1-3 independently selected R^{A}; wherein Ring A is a C5-C6 cycloalkyl or 5-6 membered heterocyclyl. In some embodiments, R² is

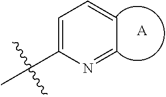

substituted with 1-3 independently selected R^{A}; wherein Ring A is a C5-C6 cycloalkyl or 5-6 membered heterocyclyl. In some embodiments, R² is

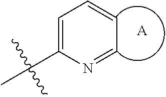

optionally substituted with 3 independently selected R^{A}; wherein Ring A is a C5-C6 cycloalkyl or 5-6 membered heterocyclyl. In some embodiments, R² is

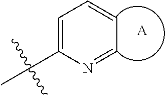

optionally substituted with 1 or 2 independently selected R^{A}; wherein Ring A is a C5-C6 cycloalkyl or 5-6 membered heterocyclyl. In some embodiments, R² is

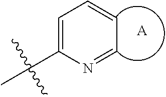

optionally substituted with 2 independently selected R^{A}; wherein Ring A is a C5-C6 cycloalkyl or 5-6 membered heterocyclyl. In some embodiments, R² is

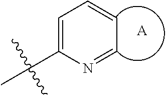

optionally substituted with one R^{A}; wherein Ring A is a C5-C6 cycloalkyl or 5-6 membered heterocyclyl. In some embodiments, Ring A is substituted and the pyridinyl ring is unsubstituted. In some embodiments, Ring A is unsubstituted and the pyridinyl ring is substituted. In some embodiments, both Ring A and the pyridinyl ring are substituted. In some embodiments, both Ring A and the pyridinyl ring are unsubstituted.

In some embodiments, Ring A is a C5-C6 cycloalkyl. In some embodiments, Ring A is cyclopentyl. In some embodiments, Ring A is cyclopentyl substituted with 1 or 2 independently selected R^{A}. In some embodiments, Ring A is cyclopentyl substituted with 2 geminal R^{A}. In some embodiments, Ring A is cyclopentyl substituted with 2 geminal R^{A} that are the same. In some embodiments, Ring A is cyclopentyl substituted with 2 geminal R^{A} that are different.

In some embodiments, Ring A is a 5-6 membered heterocyclyl. In some embodiments, Ring A is a 5-6 membered heterocyclyl containing a nitrogen atom. In some embodiments, Ring A is a piperidinyl substituted with 1 or 2 independently selected R^{A}. In some embodiments, Ring A is a piperidinyl substituted with one R^{A}.

In some embodiments, R^{A} is halogen. In some embodiments, R^{A} is fluoro or chloro. In some embodiments, R^{A} is cyano. In some embodiments, R^{A} is —N═S(O)(Me)₂.

In some embodiments, R^{A} is —NR^{B}R^{C}.

In some embodiments, R^{A} is —C(═O)NR^{B}R^{C}.

In some embodiments, R^{A} is C1-C6 alkyl substituted with hydroxyl or —NR^{B}R^{C}. In some embodiments, R^{A} is C1-C6 alkyl substituted with hydroxyl. In some embodiments, R^{A} is 2-hydroxy-2-propyl. In some embodiments, R^{A} is C1-C6 alkyl substituted with —NR^{B}R^{C}. In some embodiments, R^{A} is 2-amino-2-propyl. In some embodiments, R^{A} is —CH₂NR^{B}CH₃ or —CH(CH₃)NR^{B}CH₃; wherein R^{B} is hydrogen or methyl. In some embodiments, R^{A} is methyl.

In some embodiments, R^{A} is C3-C6 cycloalkyl optionally substituted with —NR^{B}R^{C}. In some embodiments, R^{A} is C3-C6 cycloalkyl substituted with —NR^{B}R^{C}. In some embodiments, R^{A} is cyclopropyl optionally substituted with —NR^{B}R^{C}. In some embodiments, R^{A} is an unsubstituted C3-C6 cycloalkyl. In some embodiments, R^{A} is cyclopropyl.

In some embodiments, R^{A} is 4-6 membered heterocyclyl optionally substituted with 1-2 substituents independently selected from halogen and C1-C6 alkyl. In some embodiments, R^{A} is 4-6 membered heterocyclyl substituted with 1-2 substituents independently selected from halogen and C1-C6 alkyl.

In some embodiments, one R^{A} is selected from the group consisting of azetidinyl, oxetanyl, pyrrolidinyl, 2-pyrrolidinone, oxazolidin-2-onyl, morpholinyl, piperazinyl, and piperidinyl; each optionally substituted with halogen or C1-C3 alkyl. In some embodiments, one R^{A} is selected from azetidinyl, oxetanyl, pyrrolidinyl, 2-pyrrolidinone, oxazolidin-2-onyl, morpholinyl, piperazinyl, and piperidinyl; each substituted with halogen or C1-C3 alkyl. In some embodiments, one R^{A} is selected from azetidinyl, oxetanyl, pyrrolidinyl, 2-pyrrolidinone, oxazolidin-2-onyl, morpholinyl, piperazinyl, and piperidinyl; each optionally substituted with fluoro or methyl. In some embodiments, one R^{A} is selected from azetidinyl, oxetanyl, pyrrolidinyl, 2-pyrrolidinone, oxazolidin-2-onyl, morpholinyl, piperazinyl, and piperidinyl; each substituted with fluoro or methyl. In some embodiments, the azetidinyl, oxetanyl, pyrrolidinyl, 2-pyrrolidinone, oxazolidin-2-onyl, morpholinyl, piperazinyl, or piperidinyl of one R^{A} is substituted on a carbon atom. In some embodiments, the azetidinyl, pyrrolidinyl, oxazolidin-2-onyl, morpholinyl, piperazinyl, or piperidinyl of one R^{A} is substituted on a nitrogen atom. In some embodiments, one R^{A} is an unsubstituted 5-6 membered heterocyclyl. In some embodiments, one R^{A} is selected from unsubstituted azetidinyl, oxetanyl, pyrrolidinyl, 2-pyrrolidinone, oxazolidin-2-onyl, morpholinyl, piperazinyl, and piperidinyl.

In some embodiments, R^{A} is a 4 membered heterocyclyl optionally substituted with 1-2 substituents independently selected from halogen and C1-C6 alkyl. In some embodiments, R^{A} is a 4 membered heterocyclyl optionally substituted with 1-2 substituents independently selected from fluoro and methyl. In some embodiments, R^{A} is azetidinyl optionally substituted with 1-2 substituents independently selected from fluoro and methyl.

In some embodiments, R^{A} is 5 membered heterocyclyl optionally substituted with 1-2 substituents independently selected from halogen and C1-C6 alkyl. In some embodiments, R^{A} is a 5 membered heterocyclyl optionally substituted with 1-2 substituents independently selected from fluoro and methyl. In some embodiments, R^{A} is pyrrolidinyl optionally substituted with 1-2 substituents independently selected from fluoro and methyl. In some embodiments, R^{A} is oxazolidin-2-onyl optionally substituted with 1-2 substituents independently selected from fluoro and methyl. In some embodiments, R^{A} is 6 membered heterocyclyl optionally substituted with 1-2 substituents independently selected from halogen and C1-C6 alkyl. In some embodiments, R^{A} is a 6 membered heterocyclyl optionally substituted with 1-2 substituents independently selected from fluoro and methyl. In some embodiments, R^{A} is selected from the group consisting of piperidinyl, piperazinyl, and morpholinyl, wherein each R^{A} is optionally substituted with 1-2 substituents independently selected from halogen and C1-C6 alkyl. In some embodiments, R^{A} is piperidinyl optionally substituted with 1-2 substituents independently selected from fluoro and methyl. In some embodiments, R^{A} is piperazinyl optionally substituted with 1-2 substituents independently selected from fluoro and methyl. In some embodiments, R^{A} is morpholinyl optionally substituted with 1-2 substituents independently selected from fluoro and methyl. In some embodiments, R^{A} is an unsubstituted piperidinyl, piperazinyl, or morpholinyl.

In some embodiments, R^{B} and R^{C} are independently hydrogen or C1-C6 alkyl. In some embodiments, R^{B} and R^{C} are the same. In some embodiments, R^{B} and R^{C} are different. In some embodiments, R^{B} and R^{C} are each hydrogen. In some embodiments, one of R^{B} and R^{C} is hydrogen and the other of R^{B} and R^{C} is C1-C6 alkyl. In some embodiments, one of R^{B} and R^{C} is hydrogen and the other of R^{B} and R^{C} is methyl. In some embodiments, R^{B} and R^{C} are each an independently selected C1-C6 alkyl. In some embodiments, R^{B} and R^{C} are each methyl.

In some embodiments, R² is selected from the group consisting of:

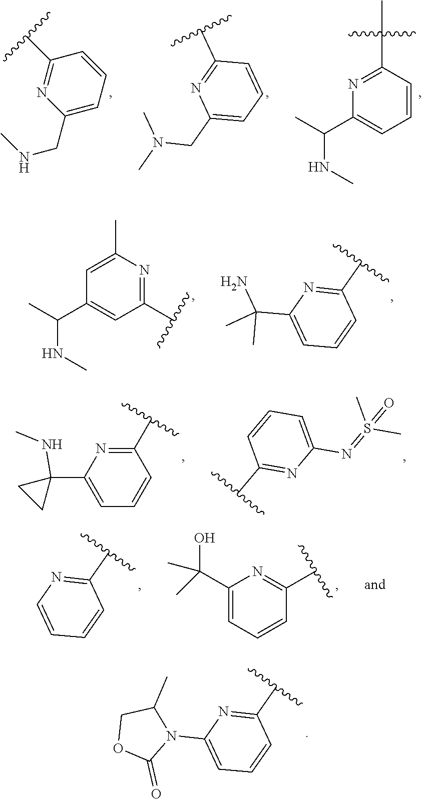

In some embodiments, R² is selected from the group consisting of:

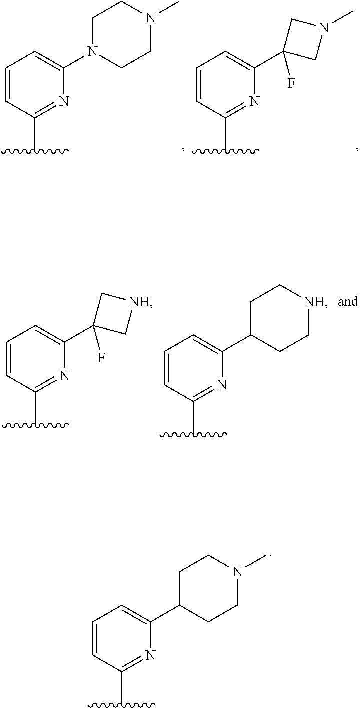

In some embodiments, R² is selected from the group consisting of:

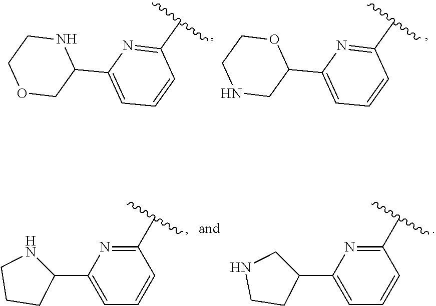 [SMILES: *c1cccc(C2COCCN2)n1 ; *c1cccc(C2CNCCO2)n1 ; *c1cccc(C2CCCN2)n1 ; *c1cccc(C2CCNC2)n1]

In some embodiments, R² is selected from the group consisting of:

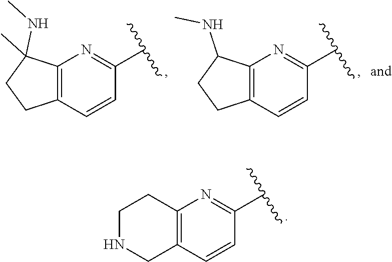

In some embodiments, R² is selected from the group consisting of:

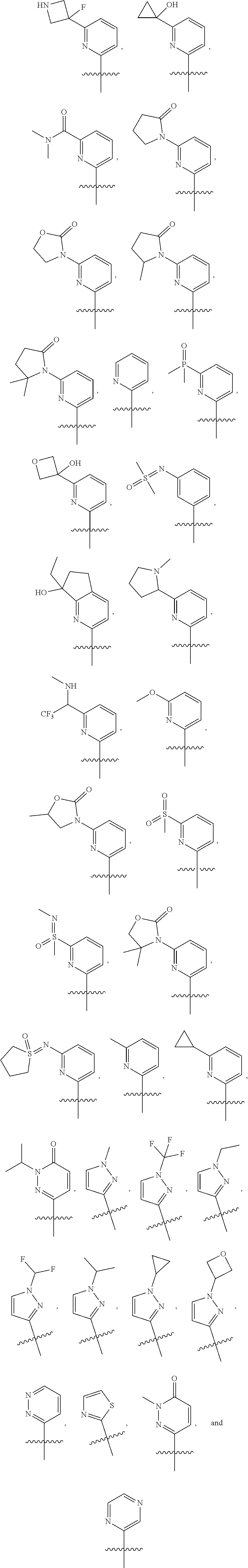

In some embodiments, R² is C1-C6 alkyl. In some embodiments, R² is C1-C3 alkyl. In some embodiments, R² is methyl.

In some embodiments, R² is hydrogen.

In some embodiments, R³ is C1-C6 alkyl. In some embodiments, R³ is C1-C3 alkyl. In some embodiments, R³ is methyl.

In some embodiments, R³ is hydrogen.

In some embodiments, R⁴ is
(i) phenyl optionally substituted with 1 or 2 substituents each independently selected from the group consisting of: halogen, cyano, —SO₂(C1-C6 alkyl), C1-C6 haloalkyl, C1-C6 deuteroalkyl, C1-C6 alkyl optionally substituted with 1 or 2 substituents each independently selected from: —NR^{B}R^{C} and —CO₂H; —(C1-C6 alkyl)ₙ-C(═O)NR^{E}R^{F}, C3-C6 cycloalkyl optionally substituted with C1-C6 alkyl; 5-6 membered heteroaryl optionally substituted with C1-C6 alkyl; 4-12 membered heterocyclyloxy optionally substituted with 1 or 2 independently selected R^{G}; and 4-12 membered heterocyclyl optionally substituted with 1 or 2 independently selected R^{G};
(ii) 9-12 membered heterocyclyl optionally substituted with 1-3 independently selected C1-C6 alkyl;
(iii) 5-10 membered heteroaryl optionally substituted with 1 or 2 substituents independently selected from the group consisting of C1-C6 alkyl; C1-C6 haloalkyl, C1-C6 deuteroalkyl, C3-C6 cycloalkyl, 5-6 membered heteroaryl optionally substituted with C1-C6 alkyl; 4-12 membered heterocyclyloxy optionally substituted with 1 or 2 independently selected R^{G}; and 4-12 membered heterocyclyl optionally substituted with 1 or 2 C1-C6 alkyl or amino;
(iv) C3-C6 cycloalkyl; or
(v) C(O)—R^{I}.

In some embodiments, R⁴ is phenyl optionally substituted with 1 or 2 substituents each independently selected from the group consisting of halogen, cyano, —SO₂(C1-C6 alkyl), C1-C6 haloalkyl, C1-C6 deuteroalkyl, C1-C6 alkyl optionally substituted with 1 or 2 substituents independently selected from —NR^{B}R^{C} and —CO₂H; —(C1-C6 alkyl)ₙ-C(═O)NR^{E}R^{F}, C3-C6 cycloalkyl optionally substituted with C1-C6 alkyl; 5-6 membered heteroaryl optionally substituted with C1-C6 alkyl; 4-12 membered heterocyclyloxy optionally substituted with 1 or 2 independently selected R^{G}; and 4-12 membered heterocyclyl optionally substituted with 1 or 2 independently selected R^{G}.

In some embodiments, R⁴ is phenyl substituted with 1 or 2 substituents independently selected from the group consisting of halogen, cyano, —SO₂(C1-C6 alkyl), C1-C6 haloalkyl, C1-C6 deuteroalkyl, C1-C6 alkyl optionally substituted with 1 or 2 substituents independently selected from —NR^{B}R^{C} and —CO₂H; —(C1-C6 alkyl)ₙ-C(═O)NR^{E}R^{F}, C3-C6 cycloalkyl optionally substituted with C1-C6 alkyl; 5-6 membered heteroaryl optionally substituted with C1-C6 alkyl; 4-12 membered heterocyclyloxy optionally substituted with 1 or 2 independently selected R^{G}; and 4-12 membered heterocyclyl optionally substituted with 1 or 2 independently selected R^{G}.

In some embodiments, R⁴ is phenyl substituted with 2 substituents independently selected from the group consisting of halogen, cyano, —SO₂(C1-C6 alkyl), C1-C6 haloalkyl, C1-C6 deuteroalkyl, C1-C6 alkyl optionally substituted with 1 or 2 substituents independently selected from —NR^{B}R^{C} and —CO₂H; —(C1-C6 alkyl)ₙ-C(═O)NR^{E}R^{F}, C3-C6 cycloalkyl optionally substituted with C1-C6 alkyl; 5-6 membered heteroaryl optionally substituted with C1-C6 alkyl; 4-12 membered heterocyclyloxy optionally substituted with 1 or 2 independently selected R^{G}; and 4-12 membered heterocyclyl optionally substituted with 1 or 2 independently selected R^{G}.

In some embodiments, R⁴ is phenyl substituted with 1 substituent selected from the group consisting of halogen, cyano, —SO₂(C1-C6 alkyl), C1-C6 haloalkyl, C1-C6 deuteroalkyl, C1-C6 alkyl optionally substituted with 1 or 2 substituents independently selected from —NR^{B}R^{C} and —CO₂H; —(C1-C6 alkyl)ₙ-C(═O)NR^{E}R^{F}, C3-C6 cycloalkyl optionally substituted with C1-C6 alkyl; 5-6 membered heteroaryl optionally substituted with C1-C6 alkyl; 4-12 membered heterocyclyloxy optionally substituted with 1 or 2 independently selected R^{G}; and 4-12 membered heterocyclyl optionally substituted with 1 or 2 independently selected R^{G}.

In some embodiments, R⁴ is selected from the group consisting of:

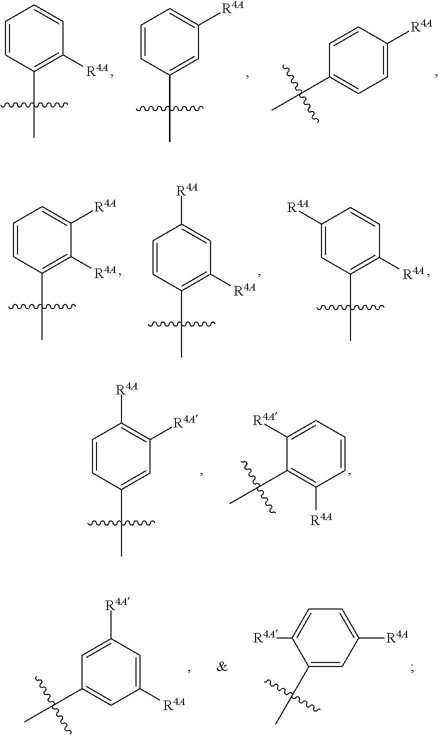

wherein each R^{4A} and R^{4A′} are independently selected from the group consisting of halogen, cyano, —SO₂(C1-C6 alkyl), C1-C6 alkyl optionally substituted with 1 or 2 substituents independently selected from —NR^{B}R^{C} and —CO₂H; —(C1-C6 alkyl)ₙ-C(═O)NR^{E}R^{F}, C3-C6 cycloalkyl optionally substituted with C1-C6 alkyl; 5-6 membered heteroaryl optionally substituted with C1-C6 alkyl; 4-12 membered heterocyclyloxy optionally substituted with 1 or 2 independently selected R^{G}; and 4-12 membered heterocyclyl optionally substituted with 1 or 2 independently selected R^{G}.

In some embodiments, R⁴ is phenyl substituted with 2 independently selected C1-C6 alkyl. In some embodiments, R⁴ is phenyl substituted with one C1-C6 alkyl. In some embodiments, R⁴ is phenyl substituted with t-butyl.

In some embodiments, R⁴ is phenyl substituted with 1 or 2 independently selected C1-C6 alkyl optionally substituted with 1 or 2 substituents independently selected from —NR^{B}R^{C} and —CO₂H. In some embodiments, R⁴ is phenyl substituted with 1 or 2 substituents independently selected from the group consisting of —C(CH₃)₂CO₂H and —CH₂CH(NCH₃R^{C})CO₂H, wherein R^{C} is selected from hydrogen and methyl.

In some embodiments, R⁴ is phenyl substituted with 1 or 2 substituents independently selected from the group consisting of halogen, —SO₂(C1-C6 alkyl), and C1-C6 alkyl. In some embodiments, R⁴ is phenyl substituted with 1 or 2 independently selected halogen. In some embodiments, R⁴ is phenyl substituted with —SO₂(C1-C6 alkyl). In some embodiments, R⁴ is phenyl substituted with 2 substituents independently selected from halogen and —SO₂(C1-C6 alkyl). In some embodiments, R⁴ is phenyl substituted with 2 substituents independently selected from halogen and C1-C6 alkyl. In some embodiments, R⁴ is phenyl substituted with 2 substituents independently selected from —SO₂(C1-C6 alkyl) and C1-C6 alkyl. In some embodiments, R⁴ is phenyl substituted with —SO₂CH₃.

In some embodiments, R⁴ is phenyl substituted with 1 or 2 substituents independently selected from the group consisting of halogen and —(C1-C6 alkyl)ₙ-C(═O)NR^{E}R^{F}. In some embodiments, R⁴ is phenyl substituted with 1 or 2 substituents independently selected from the group consisting of fluoro and —(C1-C3 alkyl)ₙ-C(═O)NR^{E}R^{F}. In some embodiments, R⁴ is phenyl substituted with —(C1-C6 alkyl)ₙ-C(═O)NR^{E}R^{F}, wherein the C1-C6 alkyl is a branched alkyl. In some embodiments, R⁴ is phenyl substituted with —(C1-C6 alkyl)ₙ-C(═O)NR^{E}R^{F}, wherein C1-C6 alkyl is —C(C1-C3 alkyl)₂-. In some embodiments, R⁴ is phenyl substituted with —C(═O)NR^{E}R^{F} or —C(CH₃)₂C(═O)NR^{E}R^{F}. In some embodiments, R⁴ is phenyl substituted with 1 or 2 substituents independently selected from the group consisting of fluoro, —CO₂NHCH₃, —CO₂N(CH₃)₂, and —C(CH₃)₂CO₂N(CH₃)₂.

In some embodiments, R⁴ is

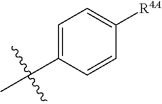

wherein R^{4A} is —(C1-C3 alkyl)ₙ-C(═O)NR^{E}R^{F}. In some embodiments, R⁴ is

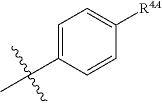

wherein R^{4A} is —C(═O)NR^{E}R^{F} or —C(CH₃)₂C(═O)NR^{E}R^{F}.

In some embodiments, R⁴ is

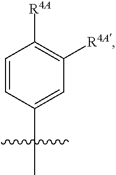

wherein R^{4A} is —(C1-C3 alkyl)ₙ-C(═O)NR^{E}R^{F} and R^{4A′} is halogen. In some embodiments, R⁴ is

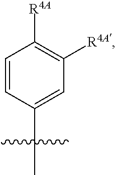

wherein R^{4A} is —C(═O)NR^{E}R^{F} and R^{4A′} is fluoro.

In some embodiments, n is 0. In some embodiments, n is 1.

In some embodiments, R^{E} and R^{F} are each independently hydrogen or C1-C6 alkyl. In some embodiments, R^{E} and R^{F} are the same. In some embodiments, R^{E} and R^{F} are different. In some embodiments, one of R^{E} and R^{F} is hydrogen and the other of R^{E} and R^{F} is C1-C6 alkyl. In some embodiments, one of R^{E} and R^{F} is hydrogen and the other of R^{E} and R^{F} is C1-C3 alkyl. In some embodiments, one of R^{E} and R^{F} is hydrogen and the other of R^{E} and R^{F} is methyl. In some embodiments, R^{E} and R^{F} are independently selected C1-C6 alkyl. In some embodiments, R^{E} and R^{F} are independently selected C1-C3 alkyl. In some embodiments, R^{E} and R^{F} are both methyl. In some embodiments, R^{E} and R^{F} are both hydrogen.

In some embodiments, R^{E} and R^{F}, together with the nitrogen atom to which they are attached, form a 4-8 membered heterocyclyl optionally substituted with C1-C6 alkyl. In some embodiments, R^{E} and R^{F}, together with the nitrogen atom to which they are attached, form a 4-6 membered heterocyclyl optionally substituted with C1-C6 alkyl. In some embodiments, R^{E} and R^{F}, together with the nitrogen atom to which they are attached, form a ring selected from the group consisting of morpholinyl, thiomorpholinyl, piperazinyl, piperdidinyl, pyrrolininyl, and azedidinyl, each optionally substituted with C1-C6 alkyl. In some embodiments, R^{E} and R^{F}, together with the nitrogen atom to which they are attached, form an unsubstituted 4-8 membered heterocyclyl. In some embodiments, R^{E} and R^{F}, together with the nitrogen atom to which they are attached, form morpholinyl, thiomorpholinyl, piperazinyl, piperdidinyl, pyrrolininyl, or azedidinyl. In some embodiments, R^{E} and R^{F}, together with the nitrogen atom to which they are attached, form morpholinyl.

In some embodiments, R⁴ is phenyl substituted with C3-C6 cycloalkyl optionally substituted with C1-C6 alkyl. In some embodiments, R⁴ is phenyl substituted with C3-C6 cycloalkyl substituted with C1-C6 alkyl. In some embodiments, R⁴ is phenyl substituted with C3-C6 cycloalkyl substituted with C1-C3. In some embodiments, R⁴ is phenyl substituted with cyclobutyl optionally substituted with methyl.

In some embodiments, R⁴ is phenyl substituted with an unsubstituted C3-C6 cycloalkyl.

In some embodiments, R⁴ is

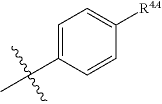

wherein R^{4A} is C3-C6 cycloalkyl optionally substituted with C1-C6 alkyl. In some embodiments, R⁴ is

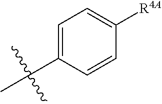

wherein R^{4A} is C3-C4 cycloalkyl optionally substituted with C1-C3 alkyl. In some embodiments, R⁴ is

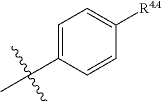

wherein R^{4A} is cyclobutyl optionally substituted with methyl.

In some embodiments, R⁴ is

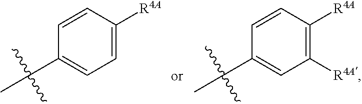

wherein R^{4A} is 5-6 membered heteroaryl optionally substituted with C1-C6 alkyl, and R^{4A′} is independently selected from R^{4A}.

In some embodiments, R⁴ is

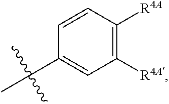

wherein R^{4A} is 5-6 membered heteroaryl optionally substituted with C1-C6 alkyl, and R^{4A′} is independently selected from halo and C1-C6 alkyl.

In some embodiments, R⁴ is

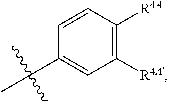

wherein R^{4A} is imidazolyl or pyrazolyl optionally substituted with C1-C3 alkyl, and R^{4A′} is independently selected from fluoro, chloro and methyl.

In some embodiments, R⁴ is selected from the group consisting of

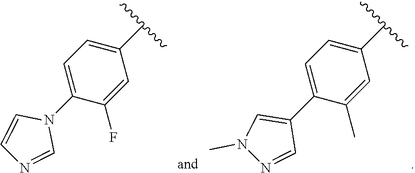 [SMILES: *c1ccc(-n2ccnc2)c(F)c1 ; *c1ccc(-c2cnn(C)c2)c(C)c1]

In some embodiments, R⁴ is

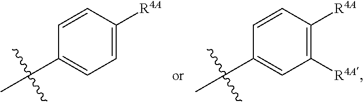

wherein R^{4A} is 4-12 membered heterocyclyloxy optionally substituted with 1 or 2 independently selected R^{G}, and R^{4A′} is independently selected from R^{4A}.

In some embodiments, R⁴ is

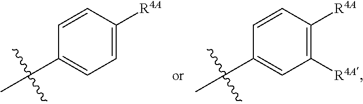

wherein R^{4A} is selected from the group consisting of piperidinyl, piperazinyl, morpholinyl, pyrrolidinyl, pyrrolidinonyl, tetrahydrofuranyl, tetrahydropyranyl, oxetanyl, azetidinyl, 1,4-oxazepan-4-yl, 2-oxa-5-azabicyclo[2.2.1]heptanyl, (1R,5S)-3-oxa-8-azabicyclo[3.2.1]octanyl, 2,5-diazabicyclo[2.2.1]heptanyl, 1,4-diazepanyl, 2-oxopiperazinyl, 3-oxopiperazinyl, and thiomorpholinyl-1-oxide, each optionally substituted with 1 or 2 independently selected R^{G}; and R^{4A′} is independently selected from fluoro, chloro, and C1-C3 alkyl.

In some embodiments, R⁴ is phenyl substituted with 1 or 2 substituents independently selected from the group consisting of halogen, C1-C6 alkyl, and 4-12 membered heterocyclyl optionally substituted with 1 or 2 independently selected R^{G}. In some embodiments, R⁴ is phenyl substituted with 1 or 2 substituents independently selected from the group consisting of halogen, C1-C6 alkyl, and 4-12 membered heterocyclyl substituted with 1 or 2 independently selected R^{G}.

In some embodiments, the 4-12 membered heterocyclyl is a 4-7 membered heterocyclyl. In some embodiments, the 4-12 membered heterocyclyl is an unsubstituted 4-7 membered heterocyclyl.

In some embodiments, R⁴ is phenyl substituted with 4-7 membered heterocyclyl selected from the group consisting of piperidinyl, piperazinyl, morpholinyl, pyrrolidinyl, pyrrolidinonyl, tetrahydrofuranyl, tetrahydropyranyl, oxetanyl, azetidinyl, 1,4-oxazepan-4-yl, 3-oxa-8-azabicyclo[3.2.1]octanyl, 2,5-diazabicyclo[2.2.1]heptanyl, 2-oxa-5-azabicyclo[2.2.1]heptanyl, 1,4-diazepanyl, 2-oxopiperazinyl, 3-oxopiperazinyl, thiomorpholinyl, and thiomorpholinyl-1-oxide, wherein each 4-7 membered heterocyclyl is optionally substituted with 1 or 2 independently selected R^{G}.

In some embodiments, R⁴ is

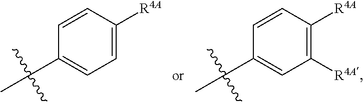

wherein R^{4A} is 4-12 membered heterocyclyl optionally substituted with 1 or 2 independently selected R^{G}. In some embodiments, R^{4A} is 4-12 membered heterocyclyl substituted with 1 or 2 independently selected R^{G}. In some embodiments, R^{4A} is selected from the group consisting of piperidinyl, piperazinyl, morpholinyl, pyrrolidinyl, pyrrolidinonyl, tetrahydrofuranyl, tetrahydropyranyl, oxetanyl, azetidinyl, 1,4-oxazepan-4-yl, 3-oxa-8-azabicyclo[3.2.1]octanyl, 2,5-diazabicyclo[2.2.1]heptanyl, 2-oxa-5-azabicyclo[2.2.1]heptanyl, 1,4-diazepanyl, 2-oxopiperazinyl, 3-oxopiperazinyl, thiomorpholinyl, thiomorpholinyl-1-oxide, octahydropyrrolo[3,4-c]pyrrolyl and 2,6-diazaspiro[3.3]heptanyl, wherein each 4-12 membered heterocyclyl is optionally substituted with 1 or 2 independently selected R^{G}. In some embodiments, R^{4A′} is independently selected from halogen and C1-C6 alkyl. In some embodiments, R^{4A′} is independently selected from fluoro, chloro, and methyl.

In some embodiments, R⁴ is

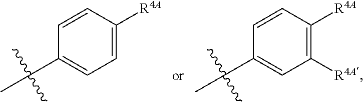

wherein R^{4A} is piperidinyl, optionally substituted with 1 or 2 independently selected R^{G}. In some embodiments, R^{4A} is piperidinyl substituted with 1 or 2 independently selected R^{G}. In some embodiments, R^{4A} is

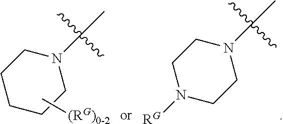

In some embodiments, R⁴ is selected from the group consisting of

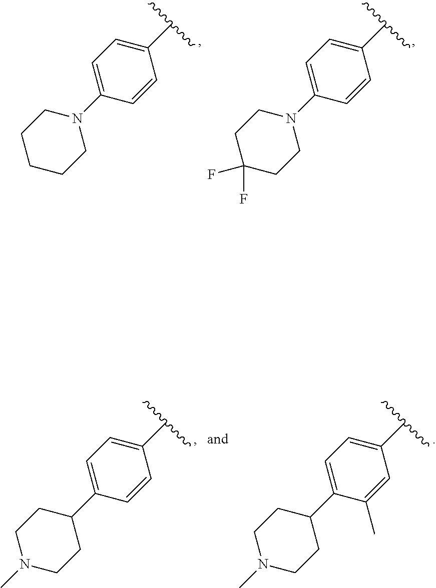 [SMILES: *c1ccc(N2CCCCC2)cc1 ; *c1ccc(N2CCC(F)(F)CC2)cc1 ; *c1ccc(C2CCN(C)CC2)cc1 ; *c1ccc(C2CCN(C)CC2)c(C)c1]

In some embodiments, R⁴ is

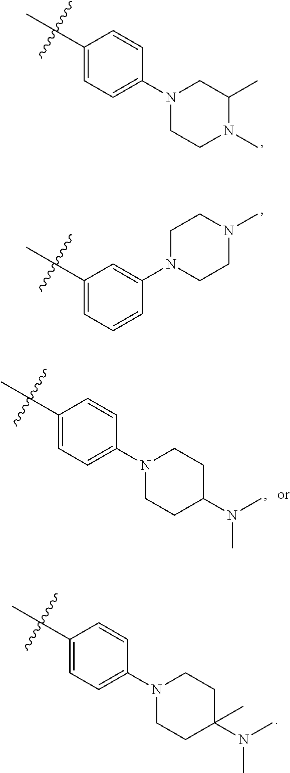 [SMILES: *c1ccc(N2CCN(C)C(C)C2)cc1 ; *c1cccc(N2CCN(C)CC2)c1 ; *c1ccc(N2CCC(N(C)C)CC2)cc1 ; *c1ccc(N2CCC(C)(N(C)C)CC2)cc1]

In some embodiments, R⁴ is

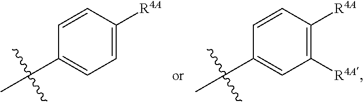

wherein R^{4A} is piperazinyl, optionally substituted with 1 or 2 independently selected R^{G}. In some embodiments, R^{4A} is piperazinyl substituted with 1 or 2 independently selected R^{G}. In some embodiments, R^{4A} is

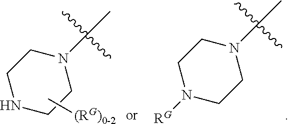

In some embodiments, R⁴ is selected from the group consisting of

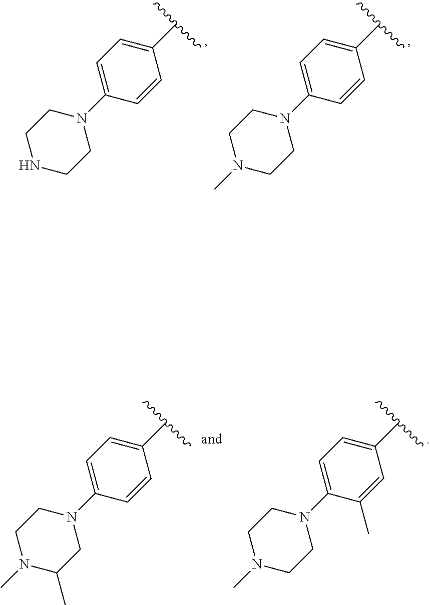 [SMILES: *c1ccc(N2CCNCC2)cc1 ; *c1ccc(N2CCN(C)CC2)cc1 ; *c1ccc(N2CCN(C)C(C)C2)cc1 ; *c1ccc(N2CCN(C)CC2)c(C)c1]

In some embodiments, R⁴ is

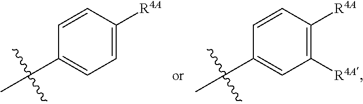

wherein R^{4A} is morpholinyl, optionally substituted with 1 or 2 independently selected R^{G}. In some embodiments, R^{4A} is

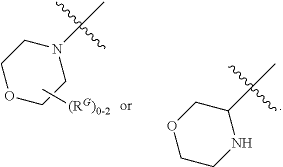

In some embodiments, R⁴ is selected from the group consisting of

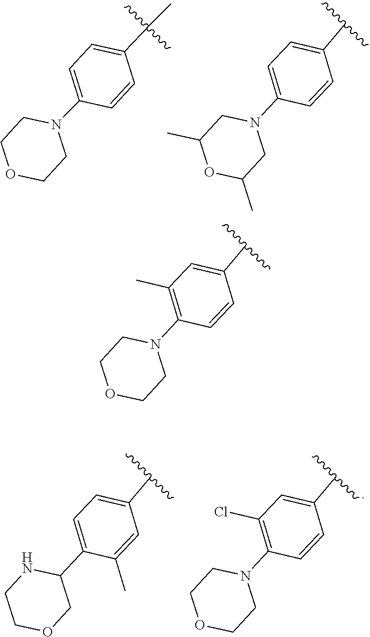

In some embodiments, R⁴ is

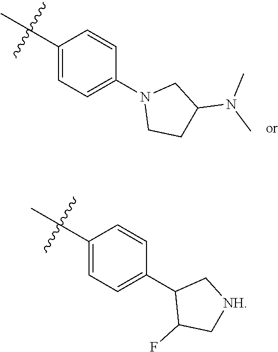 [SMILES: *c1ccc(N2CCC(N(C)C)C2)cc1 ; *c1ccc(C2CNCC2F)cc1]

In some embodiments, R⁴ is

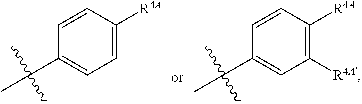

wherein R^{4A} is pyrrolidinyl, optionally substituted with 1 or 2 independently selected R^{G}. In some embodiments, R^{4A} is

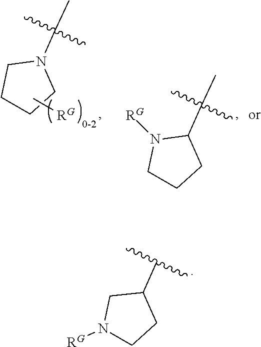

In some embodiments, R⁴ is selected from the group consisting of

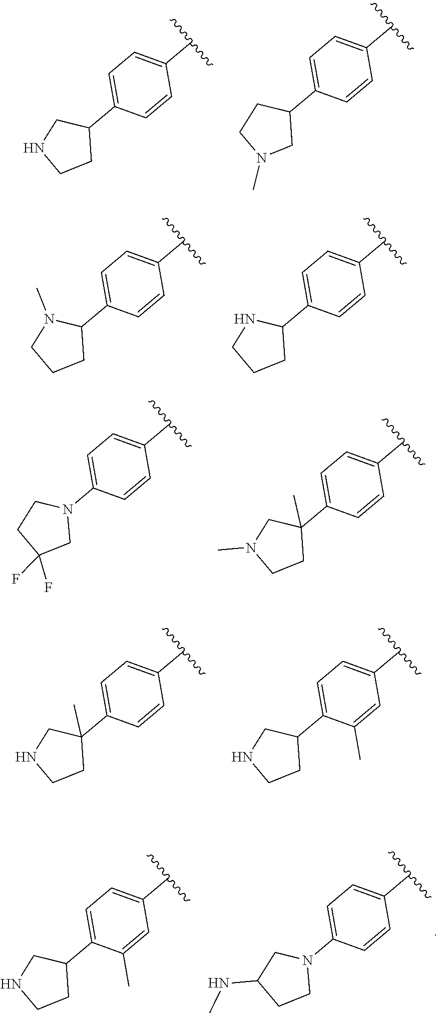

In some embodiments, R⁴ is

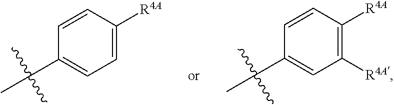

wherein R^{4A} is pyrrolidinonyl, optionally substituted with 1 or 2 independently selected R^{G}. In some embodiments, R^{4A} is selected from the group consisting of

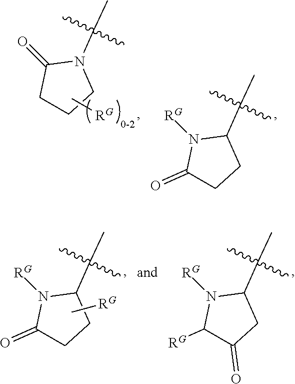

In some embodiments, R⁴ is selected from the group consisting of:

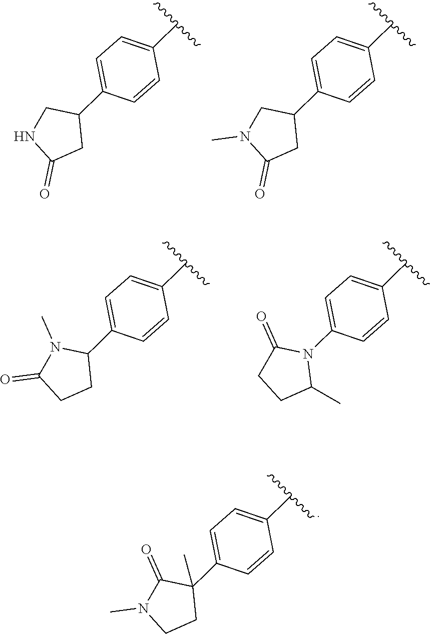 [SMILES: *c1ccc(C2CNC(=O)C2)cc1 ; *c1ccc(C2CC(=O)N(C)C2)cc1 ; *c1ccc(C2CCC(=O)N2C)cc1 ; *c1ccc(N2C(=O)CCC2C)cc1 ; *c1ccc(C2(C)CCN(C)C2=O)cc1]

In some embodiments, R⁴ is

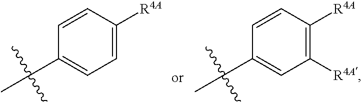

wherein R^{4A} is tetrahydrofuranyl, optionally substituted with 1 or 2 independently selected R^{G}. In some embodiments, R^{4A} is

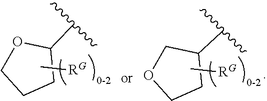

In some embodiments, R⁴ is selected from the group consisting of

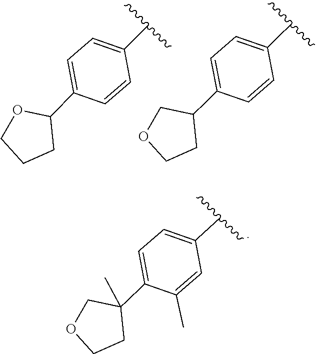

In some embodiments, R⁴ is

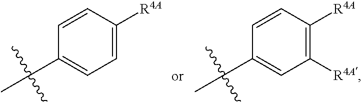

wherein R^{4A} is tetrahydropyranyl, optionally substituted with 1 or 2 independently selected R^{G}. In some embodiments, R⁴ is

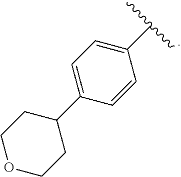 [SMILES: *c1ccc(C2CCOCC2)cc1]

In some embodiments, R⁴ is

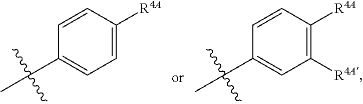

wherein R^{4A} is oxetanyl, optionally substituted with 1 or 2 independently selected R^{G}. In some embodiments, R⁴ is

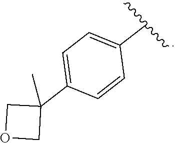 [SMILES: *c1ccc(C2(C)COC2)cc1]

In some embodiments, R⁴ is

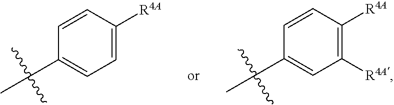

wherein R^{4A} is azetidinyl, optionally substituted with 1 or 2 independently selected R^{G}. In some embodiments, R⁴ is

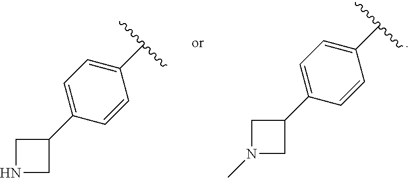 [SMILES: *c1ccc(C2CNC2)cc1 ; *c1ccc(C2CN(C)C2)cc1]

In some embodiments, the phenyl ring is further substituted by fluoro, for example,

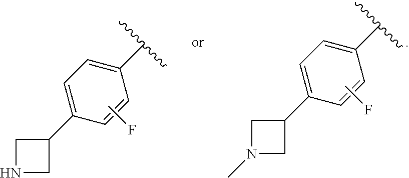

In some embodiments, R⁴ is

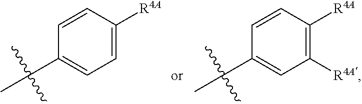

wherein R^{4A} is thiomorpholinyl-1-oxide, optionally substituted with 1 or 2 independently selected R^{G}. In some embodiments, R^{4A} is thiomorpholinyl-1-oxide, substituted with ═NR^{H}. In some embodiments, R⁴ is

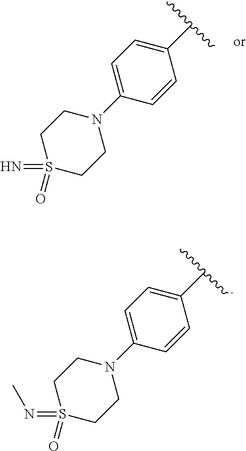 [SMILES: *c1ccc(N2CCS(=N)(=O)CC2)cc1 ; *c1ccc(N2CCS(=O)(=NC)CC2)cc1]

In some embodiments, R⁴ is

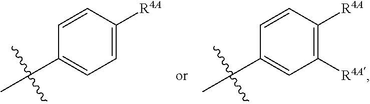

wherein R^{4A} is 1,4-oxazepan-4-yl, optionally substituted with 1 or 2 independently selected R^{G}. In some embodiments, R⁴ is

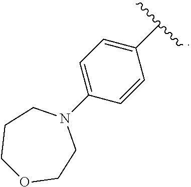 [SMILES: *c1ccc(N2CCCOCC2)cc1]

In some embodiments, R⁴ is

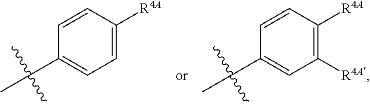

wherein R^{4A} is 1,4-diazepanyl, optionally substituted with 1 or 2 independently selected R^{G}. In some embodiments, R⁴ is

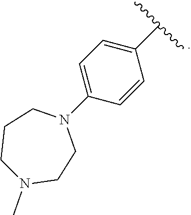 [SMILES: *c1ccc(N2CCCN(C)CC2)cc1]

In some embodiments, R⁴ is

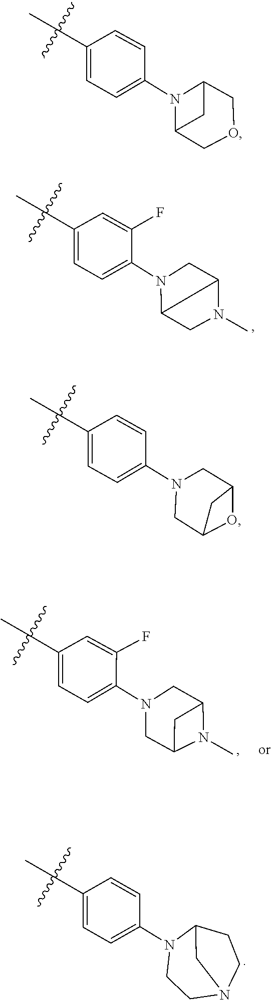 [SMILES: *c1ccc(N2C3COCC2C3)cc1 ; *c1ccc(N2CC3C2CN3C)c(F)c1 ; *c1ccc(N2CC3CC(C2)O3)cc1 ; *c1ccc(N2CC3CC(C2)N3C)c(F)c1 ; *c1ccc(N2CCN3CCC2C3)cc1]

In some embodiments, R⁴ is

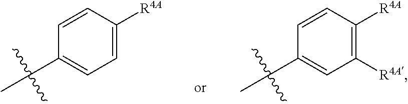

wherein R^{4A} is selected from the group consisting of 3-oxa-8-azabicyclo[3.2.1]octanyl, 2,5-diazabicyclo[2.2.1]heptanyl, and 2-oxa-5-azabicyclo[2.2.1]heptanyl. In some embodiments, R⁴ is selected form the group consisting of

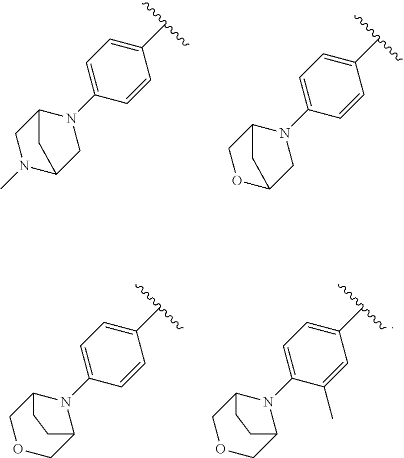 [SMILES: *c1ccc(N2CC3CC2CN3C)cc1 ; *c1ccc(N2CC3CC2CO3)cc1 ; *c1ccc(N2C3CCC2COC3)cc1 ; *c1ccc(N2C3CCC2COC3)c(C)c1]

In some embodiments, the phenyl ring is further substituted by fluoro.

In some embodiments, R⁴ is

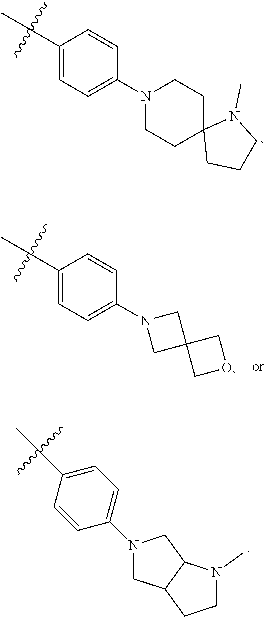 [SMILES: *c1ccc(N2CCC3(CCCN3C)CC2)cc1 ; *c1ccc(N2CC3(COC3)C2)cc1 ; "or" ; *c1ccc(N2CC3CCN(C)C3C2)cc1]

In some embodiments, In some embodiments, R⁴ is

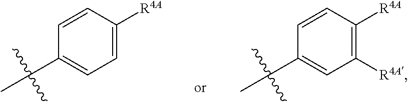

wherein R^{4A} is selected from the group consisting of octahydropyrrolo[3,4-c]pyrrolyl and 2,6-diazaspiro[3.3]heptanyl. In some embodiments, R⁴ is selected form the group consisting of:

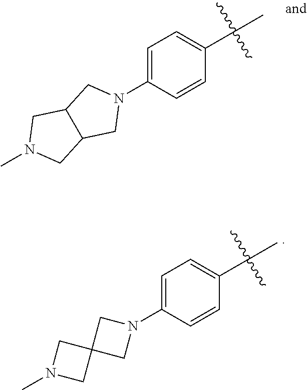 [SMILES: *c1ccc(N2CC3CN(C)CC3C2)cc1 ; *c1ccc(N2CC3(CN(C)C3)C2)cc1]

In some embodiments, R⁴ is

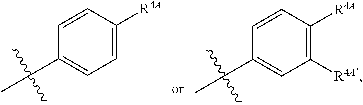

wherein R^{4A} is 2-oxopiperazinyl or 3-oxopiperazinyl. In some embodiments, R⁴ is selected form the group consisting of

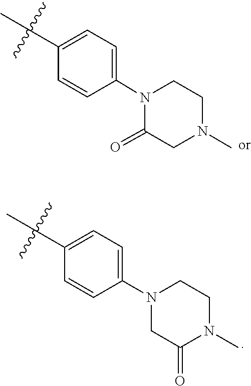 [SMILES: *c1ccc(N2CCN(C)CC2=O)cc1 ; *c1ccc(N2CCN(C)C(=O)C2)cc1]

In some embodiments, R⁴ is

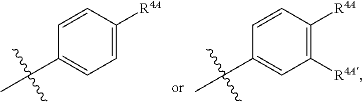

wherein R^{4A} is 4-12 membered heterocyclyloxy optionally substituted with 1 or 2 independently selected R^{G}, and R^{4A′} is independently selected from R^{4A}.

In some embodiments, R⁴ is

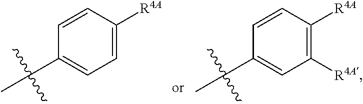

wherein R^{4A} is selected from the group consisting of azetidinyloxy, piperidinyloxy, and pyrrolidinyloxy, each optionally substituted with 1 or 2 independently selected R^{G}.

In some embodiments, R⁴ is selected form the group consisting of

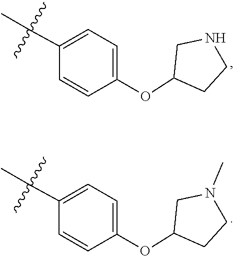 [SMILES: *c1ccc(OC2CCNC2)cc1 ; *c1ccc(OC2CCN(C)C2)cc1]

In some embodiments, R⁴ is an unsubstituted phenyl.

In some embodiments, each R^{G} is independently selected from halogen, C1-C6 alkyl, C1-C6 deuteroalkyl, —NR^{B}R^{C}, and ═NR^{H}. In some embodiments, each R^{G} is halogen or C1-C3 alkyl. In some embodiments, each R^{G} is independently selected from fluoro, chloro, and methyl. In some embodiments, each R^{G} is C1-C3 deuteroalkyl. In some embodiments, each R^{G} is —CD₃. In some embodiments, each R^{G} is —NR^{B}R^{C}. In some embodiments, each R^{G} is —NCH₃R^{C}, wherein R^{C} is selected from hydrogen and methyl. In some embodiments, each R^{G} is ═NR^{H}. In some embodiments, R^{G} is methyl. In some embodiments, two independently selected R^{G} groups are geminal.

In some embodiments, R^{H} is hydrogen. In some embodiments, R^{H} is C1-C6 alkyl. In some embodiments, R^{H} is methyl.

In some embodiments, R⁴ is a 9-12 membered heterocyclyl optionally substituted with 1-3 independently selected C1-C6 alkyl. In some embodiments, R⁴ is a 9-12 membered heterocyclyl substituted with 1-3 independently selected C1-C6 alkyl.

In some embodiments, R⁴ is

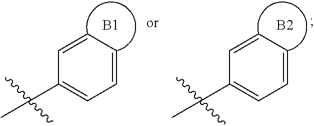

each optionally substituted with 1-3 independently selected C1-C6 alkyl. In some embodiments, Ring B1 and Ring B2 are 5-6 membered heterocyclyl groups. In some embodiments, Ring B1 and Ring B2 are 5 membered heterocyclyl. In some embodiments, Ring B1 and Ring B2 are 5 membered heterocyclyl containing one nitrogen. In some embodiments, Ring B1 and Ring B2 are 6 membered heterocyclyl. In some embodiments, Ring B1 and Ring B2 are 6 membered heterocyclyl containing one nitrogen. In some embodiments, Ring B1 and Ring B2 are 5-6 membered heterocyclyl substituted with 1-3 independently selected C1-C3 alkyl. In some embodiments, Ring B1 and Ring B2 are 5-6 membered heterocyclyl substituted with two independently selected geminal C1-C3 alkyl groups. In some embodiments, Ring B1 and Ring B2 are 5-6 membered heterocyclyl with a spiro C3-C6 cycloalkyl.

In some embodiments, Ring B1 and Ring B2 are selected from the group consisting of pyrrolidin-2-onyl, piperidin-2-onyl, piperidinyl, and pyrrolidinyl. In some embodiments, R⁴ is selected from the group consisting of

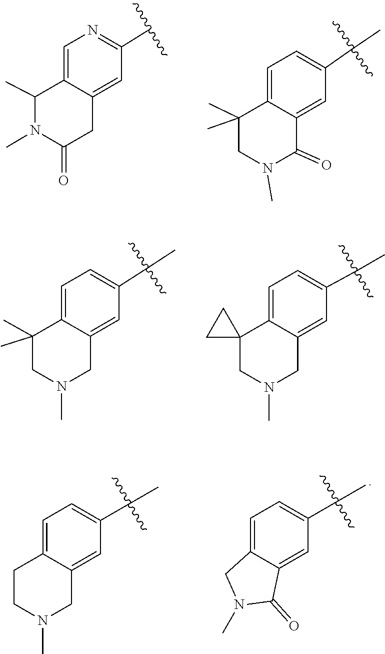 [SMILES: *c1cc2c(cn1)C(C)N(C)C(=O)C2 ; *c1ccc2c(c1)C(=O)N(C)CC2(C)C ; *c1ccc2c(c1)CN(C)CC2(C)C ; *c1ccc2c(c1)CN(C)CC21CC1 ; *c1ccc2c(c1)CN(C)CC2 ; *c1ccc2c(c1)C(=O)N(C)C2]

In some embodiments, R⁴ is an unsubstituted 9-12 membered heterocyclyl.

In some embodiments, R4 is 5-10 membered heteroaryl optionally substituted with 1 or 2 substituents independently selected from the group consisting of C1-C6 alkyl; C1-C6 haloalkyl, C1-C6 deuteroalkyl, C3-C6 cycloalkyl, 5-6 membered heteroaryl optionally substituted with C1-C6 alkyl; 4-12 membered heterocyclyloxy optionally substituted with 1 or 2 independently selected R^{G}; and 4-12 membered heterocyclyl optionally substituted with C1-C6 alkyl or amino.

In some embodiments, R⁴ is 5-6 membered heteroaryl optionally substituted with 1 or 2 substituents independently selected from the group consisting of C1-C6 alkyl; C1-C6 haloalkyl, C1-C6 deuteroalkyl, C3-C6 cycloalkyl, 5-6 membered heteroaryl optionally substituted with C1-C6 alkyl; 4-12 membered heterocyclyloxy optionally substituted with 1 or 2 independently selected R^{G}; and 4-12 membered heterocyclyl optionally substituted with C1-C6 alkyl or amino.

In some embodiments, R⁴ is a 5-6 membered heteroaryl substituted with 1 or 2 substituents independently selected from the group consisting of C1-C6 alkyl; C1-C6 haloalkyl, C1-C6 deuteroalkyl, C3-C6 cycloalkyl, 5-6 membered heteroaryl optionally substituted with C1-C6 alkyl; 4-12 membered heterocyclyloxy optionally substituted with 1 or 2 independently selected R^{G}; and 4-12 membered heterocyclyl optionally substituted with C1-C6 alkyl or amino. In some embodiments, R⁴ is a 5-6 membered heteroaryl substituted with 2 substituents independently selected from the group consisting of C1-C6 alkyl; C1-C6 haloalkyl, C1-C6 deuteroalkyl, C3-C6 cycloalkyl, 5-6 membered heteroaryl optionally substituted with C1-C6 alkyl; 4-12 membered heterocyclyloxy optionally substituted with 1 or 2 independently selected RG; and 4-12 membered heterocyclyl optionally substituted with C1-C6 alkyl or amino.

In some embodiments, R⁴ is a 5-6 membered heteroaryl substituted with C1-C6 alkyl; 5-6 membered heteroaryl optionally substituted with C1-C6 alkyl; 4-12 membered heterocyclyloxy optionally substituted with 1 or 2 independently selected R^{G}; or 4-12 membered heterocyclyl optionally substituted with C1-C6 alkyl or amino.

In some embodiments, R⁴ is a 5-6 membered heteroaryl substituted with 1 or 2 independently selected C1-C6 alkyl.

In some embodiments, R⁴ is selected from the group consisting of

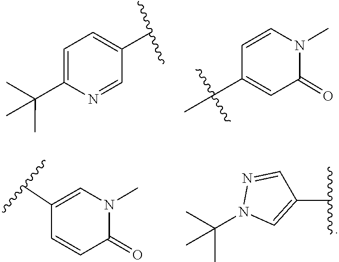

In some embodiments, R⁴ is a 5-6 membered heteroaryl substituted with 1 or 2 substituents independently selected from the group consisting of pyrazolyl and imidazolyl, each optionally substituted with C1-C6 alkyl. In some embodiments, R⁴ is selected from the group consisting of

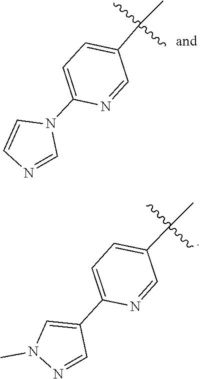 [SMILES: *c1ccc(-n2ccnc2)nc1 ; *c1ccc(-c2cnn(C)c2)nc1]

In some embodiments, R⁴ is a 5-6 membered heteroaryl substituted with 1 or 2 substituents independently selected from 4-12 membered heterocyclyloxy optionally substituted with 1 or 2 independently selected R^{G}. In some embodiments R⁴ is a 5-6 membered heteroaryl substituted with 1 or 2 substituents independently selected from the group consisting of azetidinyloxy, piperidinyloxy, and pyrrolidinyloxy, each optionally substituted with 1 or 2 independently selected R^{G}.

In some embodiments, R⁴ is a 5-6 membered heteroaryl substituted with 4-12 membered heterocyclyloxy selected from the group consisting of:

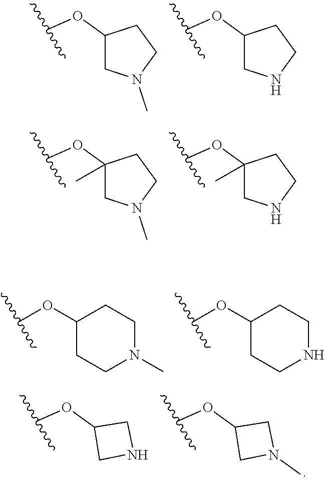

In some embodiments, R⁴ is a 5-6 membered heteroaryl substituted with a 4-12 membered heterocyclyl group optionally substituted with C1-C6 alkyl or amino. In some embodiments, R⁴ is a 5 membered heteroaryl substituted with a 5-6 membered heterocyclyl group optionally substituted with C1-C3 alkyl. In some embodiments, R⁴ is a 5 membered heteroaryl substituted with a 5-6 membered heterocyclyl group optionally substituted with methyl. In some embodiments, R⁴ is a 6 membered heteroaryl substituted with a 5-6 membered heterocyclyl group optionally substituted with C1-C3 alkyl. In some embodiments, R⁴ is a 6 membered heteroaryl substituted with a 5-6 membered heterocyclyl group optionally substituted with methyl.

In some embodiments, R⁴ is a 5-6 membered heteroaryl substituted with a 4-12 membered heterocyclyl group selected from the group consisting of piperidinyl, piperazinyl, morpholinyl, pyrrolidinyl, pyrrolidinonyl, tetrahydrofuranyl, tetrahydropyranyl, oxetanyl, 1,4-oxazepan-4-yl, 3-oxa-8-azabicyclo[3.2.1]octanyl, 2,5-diazabicyclo[2.2.1]heptanyl, 2-oxa-5-azabicyclo[2.2.1]heptanyl, thiomorpholinyl, thiomorpholinyl-1-oxide, wherein each 4-12 membered heterocyclyl is optionally substituted with C1-C3 alkyl or amino. In some embodiments, R⁴ is a 5-6 membered heteroaryl substituted with a 4-12 membered heterocyclyl group selected from the group consisting of piperidinyl, piperazinyl, and tetrahydropyranyl, optionally substituted with C1-C3 alkyl. In some embodiments, R⁴ is a 5-6 membered heteroaryl substituted with piperidinyl optionally substituted with methyl or amino. In some embodiments, R⁴ is a 5-6 membered heteroaryl substituted with piperazinyl optionally substituted with methyl or amino. In some embodiments, R⁴ is a 5-6 membered heteroaryl substituted with tetrahydropyranyl optionally substituted with methyl or amino. In some embodiments, R⁴ is a 5-6 membered heteroaryl substituted with an unsubstituted 4-12 membered heterocyclyl group.

In some embodiments, R⁴ is a 5-6 membered heteroaryl substituted with a 4-12 membered heterocyclyl group selected from the group consisting of:

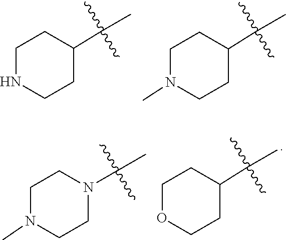

In some embodiments, R⁴ is a 5-6 membered heteroaryl substituted with 1 or 2 independently selected C1-C6 alkyl. In some embodiments, R⁴ is a 5-6 membered heteroaryl substituted with C1-C4 alkyl. In some embodiments, R⁴ is a 5-6 membered heteroaryl substituted with t-butyl.

In some embodiments, R⁴ is an unsubstituted 5-6 membered heteroaryl.

In some embodiments, R⁴ is selected from pyrazolyl, pyridyl, triazolyl, pyridazinly, and pyridonyl. In some embodiments, R⁴ is selected from the group consisting of pyrazolyl, pyridyl, triazolyl, and pyridazinyl.

In some embodiments, R⁴ is selected from the group consisting of:

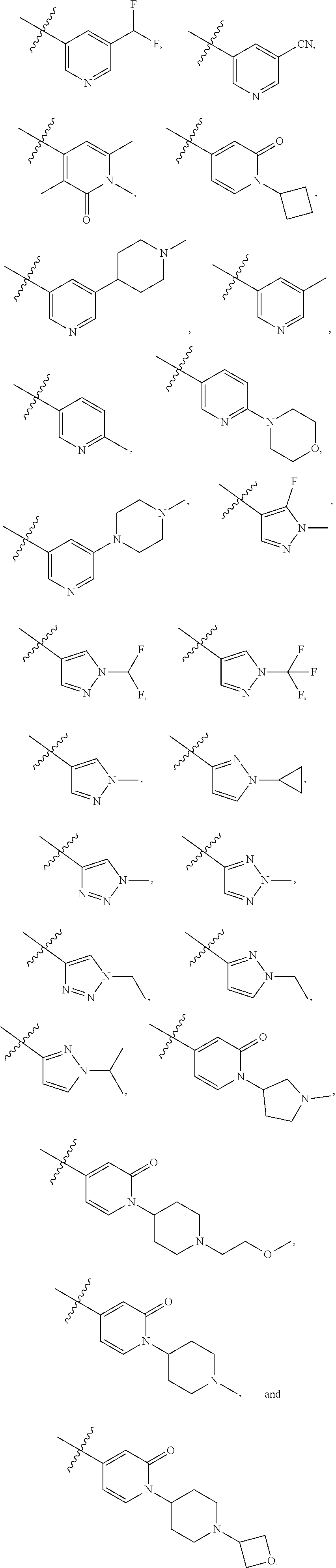

In some embodiments, R⁴ is selected from pyrazol-4-yl, 1,2,3-triazol-4-yl, pyridin-2-yl, pyridin-3-yl, and pyridin-4-yl. In some embodiments, R⁴ is selected from pyrazol-4-yl, pyrazol-3-yl, imidazolyl, isoxazolyl, thiazolyl, 1,2,3-triazol-4-yl, pyridin-3-yl, pyrazinyl, pyrimidinyl, and pyridazinyl, In some embodiments, R⁴ is selected from the group consisting of:

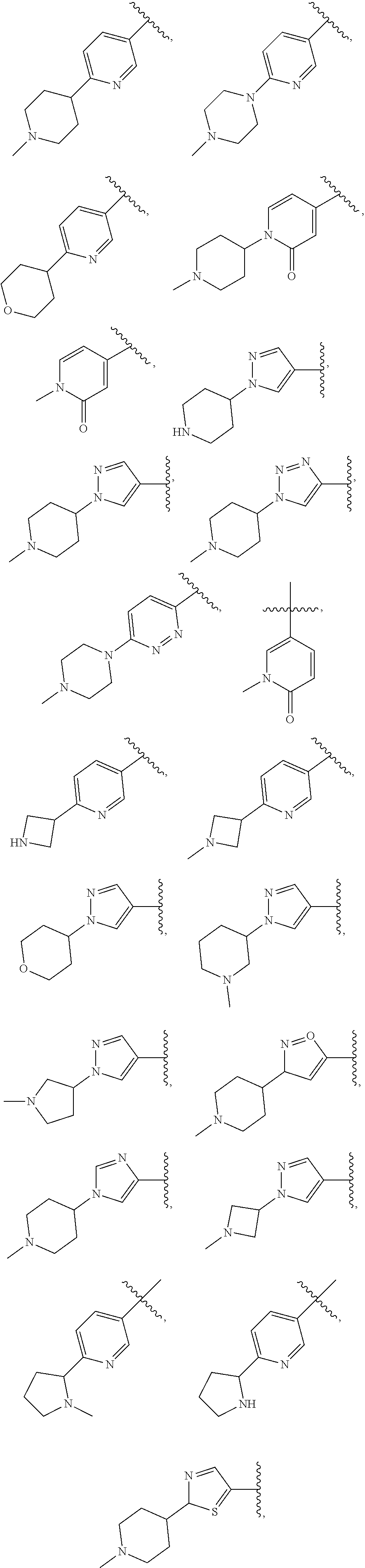

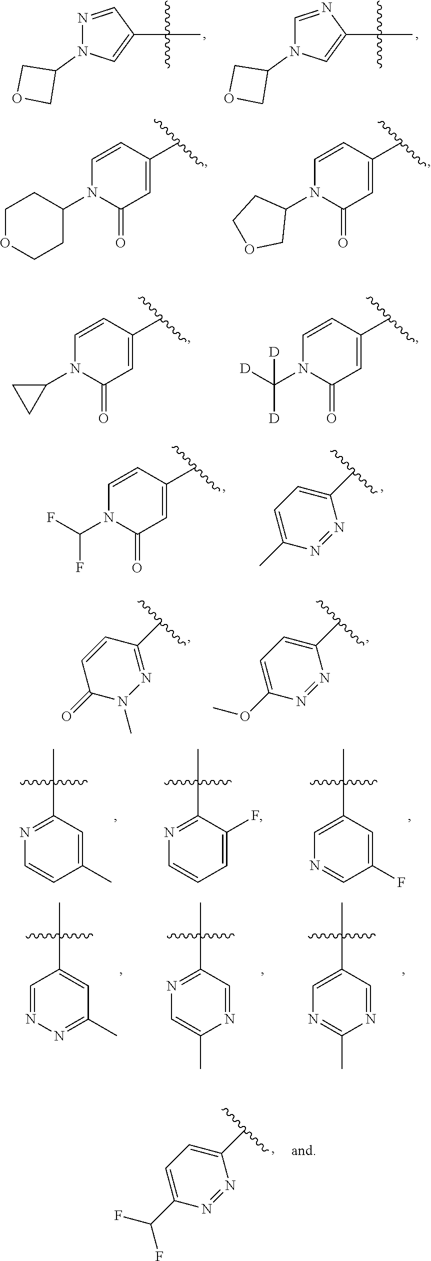

In some embodiments, R⁴ is a 9-10 membered heteroaryl optionally substituted with one or two C1-C6 alkyl. In some embodiments R⁴ is a 9-10 membered heteroaryl optionally substituted with one C1-C6 alkyl. In some embodiments R⁴ is a 9-10 membered heteroaryl optionally substituted with one C1-C3 alkyl. In some embodiments R⁴ is a 9-10 membered heteroaryl optionally substituted with one methyl. In some embodiments R⁴ is a 9-10 membered heteroaryl selected from the group consisting of:

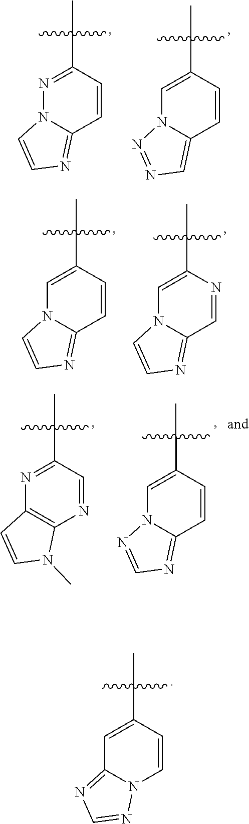

In some embodiments, R⁴ is a C3-C6 cycloalkyl. In some embodiments, R⁴ is a bridged C3-C6 cycloalkyl. In some embodiments, R⁴ is bicyclo[1.1.1]pentyl.

In some embodiments, R⁴ is C(O)—R^{I} wherein R^{I} is C1-C6 alkyl, phenyl optionally substituted with halogen; 5-6 membered heteroaryl optionally substituted with C1-C6 alkyl; C3-C6 cycloalkyl optionally substituted with 1 substituent selected from the group consisting of: halogen, phenyl, and 5-6 membered heteroaryl optionally substituted with C1-C6 alkyl.

In some embodiments, R⁴ is C(O)—R^{I} wherein R^{I} is C1-C6 alkyl. In other embodiments, R^{I} is C1-C3 alkyl. In other embodiments, R^{I} is methyl.

In some embodiments, R⁴ is C(O)—R^{I} wherein R^{I} is phenyl optionally substituted with halogen.

In some embodiments, R⁴ is C(O)—R^{I} wherein R^{I} is 5-6 membered heteroaryl optionally substituted with C1-C6 alkyl. In some embodiments, R^{I} is piperdinyl. In some embodiments, R^{I} is pyrazolyl optionally substituted with C1-C6 alkyl.

In some embodiments, R⁴ is C(O)—R^{I} wherein R^{I} is C3-C6 cycloalkyl optionally substituted with 1 substituent selected from the group consisting of: halogen, phenyl, and 5-6 membered heteroaryl optionally substituted with C1-C6 alkyl. In some embodiments R^{I} is cyclopropyl optionally substituted with 1 substituent selected from the group consisting of: halogen, phenyl, and 5-6 membered heteroaryl optionally substituted with C1-C6 alkyl. In some embodiments R^{I} is selected from the group consisting of:

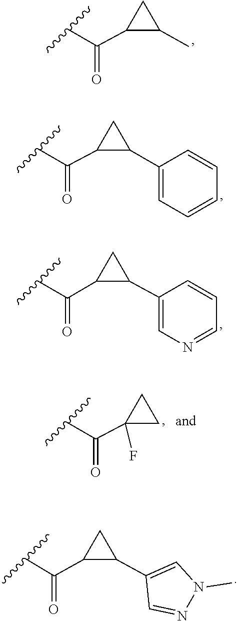

In some embodiments, R⁵ is halogen. In some embodiments, R⁵ is fluoro. In some embodiments, R⁵ is hydrogen. In some embodiments, R⁵ is C1-C6 alkyl. In some embodiments, R⁵ is C1-C3 alkyl. In some embodiments, R⁵ is methyl.

In some embodiments, R⁶ is hydrogen. In some embodiments, R⁶ is C1-C6 alkyl. In some embodiments, R⁶ is C1-C3 alkyl. In some embodiments, R⁶ is methyl.

In some embodiments, the compound of Formula (I) has the structure:

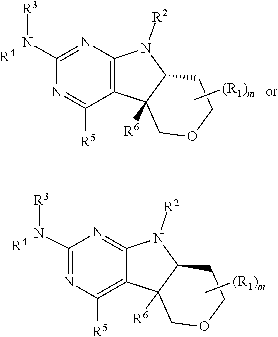

or a pharmaceutically acceptable salt thereof.

In some embodiments, the compound of Formula (I) is a compound of Formula (I-A):

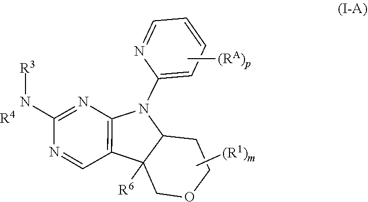

or a pharmaceutically acceptable salt thereof;
wherein:
m is 0, 1, or 2;
p is 0, 1, or 2;
each R^{A} is independently —NR^{B}R^{C}, —C(═O)NR^{B}R^{C}, C1-C6 alkyl substituted with hydroxyl or —NR^{B}R^{C}; C3-C6 cycloalkyl substituted with —NR^{B}R^{C}; —N═S(O)Me)₂, and 4-6 membered heterocyclyl optionally substituted with halogen or C1-C6 alkyl; and each R^{B} and R^{C} are independently hydrogen or C1-C6 alkyl.

In some embodiments, m is 0. In some embodiments, m is 1. In some embodiments, m is 2. In some embodiments, p is 0. In some embodiments, p is 1.

In some embodiments, the compound of Formula (I-A) has the structure:

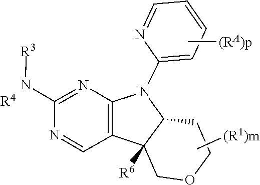

or a pharmaceutically acceptable salt thereof.

In some embodiments, the compound of Formula (I-A) has the structure:

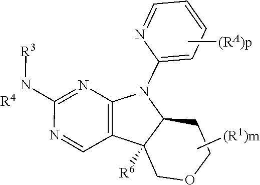

or a pharmaceutically acceptable salt thereof.

In some embodiments, the compound of Formula (I) is a compound of Formula (I-B):

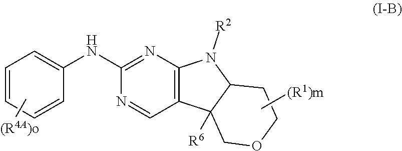

or a pharmaceutically acceptable salt thereof;
wherein:
m is 0, 1, or 2;
o is 0, 1, or 2;
each R^{4A} is independently halogen, cyano, —SO₂(C1-C6 alkyl), C1-C6 haloalkyl, C1-C6 deuteroalkyl, C1-C6 alkyl optionally substituted with 1 or 2 substituents independently selected from —NR^{B}R^{C} and —CO₂H; —(C1-C6 alkyl)ₙ₁-C(═O)NR^{E}R^{F}, C3-C6 cycloalkyl optionally substituted with C1-C6 alkyl; 5-6 membered heteroaryl optionally substituted with C1-C6 alkyl; 4-12 membered heterocyclyloxy optionally substituted with 1 or 2 independently selected R^{G}; and 4-12 membered heterocyclyl optionally substituted with 1 or 2 independently selected R^{G};
n1 is 0 or 1;
each R^{E} and R^{F} are independently hydrogen or C1-C6 alkyl;
each R^{G} is independently halogen, C1-C6 alkyl, C1-C6 deuteroalkyl, —NR^{B}R^{C}, and ═NR^{H}; and
each R^{H} is selected from hydrogen and C1-C6 alkyl.

In some embodiments, m is 0. In some embodiments, m is 1. In some embodiments, m is 2. In some embodiments, o is 0. In some embodiments, o is 1. In some embodiments, o is 2.

In some embodiments, the compound of Formula (I-B) has the structure:

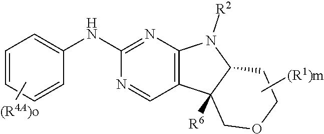

or a pharmaceutically acceptable salt thereof.

In some embodiments, the compound of Formula (I-B) has the structure:

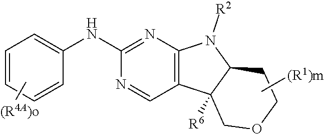

or a pharmaceutically acceptable salt thereof.

In some embodiments, the compound of Formula (I) is a compound of Formula (I-C):

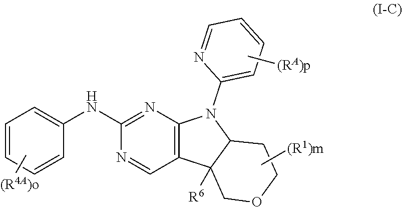

or a pharmaceutically acceptable salt thereof;
wherein:
m is 0, 1, or 2;
o is 0, 1, or 2;
p is 0, 1, or 2;
each R^{A} is independently-NR^{B}R^{C}, —C(═O)NR^{B}R^{C}, C1-C6 alkyl substituted with hydroxyl or —NR^{B}R^{C}; C3-C6 cycloalkyl substituted with —NR^{B}R^{C}; —N═S(O)Me)2, 4-6 membered heterocyclyl optionally substituted with halogen or C1-C6 alkyl;
each R^{B} and R^{C} are independently hydrogen or C1-C6 alkyl.
each R^{4A} is independently halogen, cyano, —SO₂(C1-C6 alkyl), C1-C6 haloalkyl, C1-C6 deuteroalkyl, C1-C6 alkyl optionally substituted with 1 or 2 substituents independently selected from —NR^{B}R^{C} and —CO₂H; —(C1-C6 alkyl)ₙ-C(═O)NR^{E}R^{F}; C3-C6 cycloalkyl optionally substituted with C1-C6 alkyl; 5-6 membered heteroaryl optionally substituted with C1-C6 alkyl; 4-12 membered heterocyclyloxy optionally substituted with 1 or 2 independently selected R^{G1}; and 4-12 membered heterocyclyl optionally substituted with 1 or 2 independently selected R^{G1};
n is 0 or 1;
each R^{E} and R^{F} are independently hydrogen or C1-C6 alkyl; and
each R^{G1} is independently halogen or C1-C6 alkyl.

In some embodiments, m is 0. In some embodiments, m is 1. In some embodiments, m is 2. In some embodiments, p is 0. In some embodiments, p is 1. In some embodiments, p is 2. In some embodiments, o is 0. In some embodiments, o is 1. In some embodiments, o is 2.

In some embodiments, the compound of Formula (I-C) has the structure:

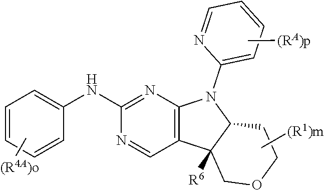

or a pharmaceutically acceptable salt thereof.

In some embodiments, the compound of Formula (I-C) has the structure:

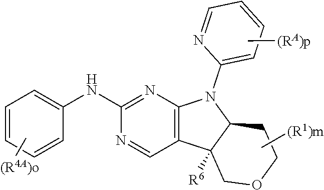

or a pharmaceutically acceptable salt thereof.

In some embodiments, the compound of Formula (I) is a compound of Formula (I-D):

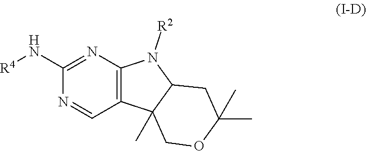

or a pharmaceutically acceptable salt thereof.

In some embodiments, the compound of Formula (I-D) has the structure:

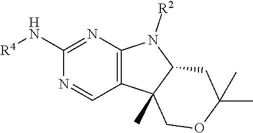

or a pharmaceutically acceptable salt thereof.

In some embodiments, the compound of Formula (I-D) has the structure:

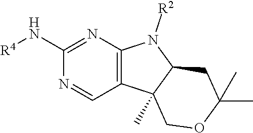

or a pharmaceutically acceptable salt thereof.

In some embodiments, the compound of Formula (I) is a compound of Formula (I-E):

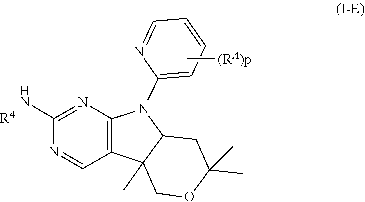

or a pharmaceutically acceptable salt thereof; wherein
p is 1 or 2;
each R^{A} of Formula (I-E) is independently —NR^{B}R^{C}, —C(═O)NR^{B}R^{C}, C1-C6 alkyl substituted with hydroxyl or —NR^{B}R^{C}; C3-C6 cycloalkyl substituted with —NR^{B}R^{C}; —N═S(O)Me)2, 4-6 membered heterocyclyl optionally substituted with halogen or C1-C6 alkyl; and
each R^{B} and R^{C} are independently hydrogen or C1-C6 alkyl.

In some embodiments, p is 1. In some embodiments, p is 2.

In some embodiments, the compound of Formula (I-E) has the structure:

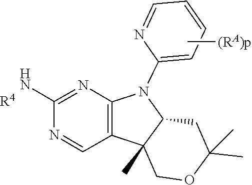

or a pharmaceutically acceptable salt thereof.

In some embodiments, the compound of Formula (I-E) has the structure:

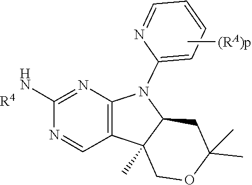

or a pharmaceutically acceptable salt thereof.

In some embodiments, the compound of Formula (I) is a compound of Formula (I-F):

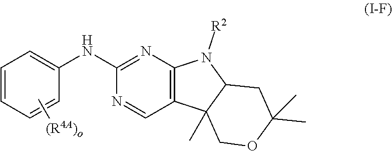

or a pharmaceutically acceptable salt thereof;
wherein o is 1 or 2;
each R^{4A} is independently halogen, cyano, —SO₂(C1-C6 alkyl), C1-C6 haloalkyl, C1-C6 deuteroalkyl, C1-C6 alkyl optionally substituted with 1 or 2 substituents independently selected from —NR^{B}R^{C} and —CO₂H; —(C1-C6 alkyl)ₙ-C(═O)NR^{E}R^{F}; C3-C6 cycloalkyl optionally substituted with C1-C6 alkyl; 5-6 membered heteroaryl optionally substituted with C1-C6 alkyl; 4-12 membered heterocyclyloxy optionally substituted with 1 or 2 independently selected R^{G1}; and 4-12 membered heterocyclyl optionally substituted with 1 or 2 independently selected R^{G1};
n is 0 or 1;
each R^{E} and R^{F} are independently hydrogen or C1-C6 alkyl; and
each R^{G1} is independently halogen or C1-C6 alkyl.

In some embodiments, o is 1. In some embodiments, o is 2.

In some embodiments, the compound of Formula (I-F) has the structure:

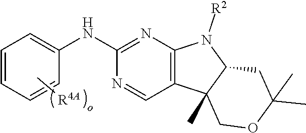

or a pharmaceutically acceptable salt thereof.

In some embodiments, the compound of Formula (I-F) has the structure:

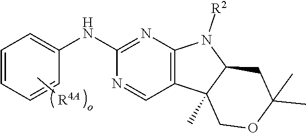

or a pharmaceutically acceptable salt thereof.

In some embodiments, the compound of Formula (I) is a compound of Formula (I-G):

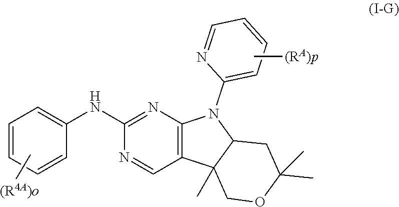

or a pharmaceutically acceptable salt thereof; wherein
p is 0, 1, or 2;
o is 1 or 2;
each R^{A} is independently halogen, cyano, —NR^{B}R^{C}, —C(═O)NR^{B}R^{C}, C1-C6 alkyl optionally substituted with hydroxyl or —NR^{B}R^{C}; C3-C6 cycloalkyl optionally substituted with —NR^{B}R^{C}; —N═S(O)Me)2, 4-6 membered heterocyclyl optionally substituted with halogen or C1-C6 alkyl;
each R^{4A} is independently halogen, cyano, —SO₂(C1-C6 alkyl), C1-C6 haloalkyl, C1-C6 deuteroalkyl, C1-C6 alkyl optionally substituted with 1 or 2 substituents independently selected from —NR^{B}R^{C} and —CO₂H; —(C1-C6 alkyl)ₙ₁-C(═O)NR^{E}R^{F}; C3-C6 cycloalkyl optionally substituted with C1-C6 alkyl; 5-6 membered heteroaryl optionally substituted with C1-C6 alkyl; 4-12 membered heterocyclyloxy optionally substituted with 1 or 2 independently selected R^{G1}; and 4-12 membered heterocyclyl optionally substituted with 1 or 2 independently selected R^{G1};
n1 is 0 or 1;
each R^{E} and R^{F} are independently hydrogen or C1-C6 alkyl; and
each R^{G1} is independently halogen or C1-C6 alkyl.

In some embodiments, p is 0. In some embodiments, p is 1. In some embodiments, p is 2. In some embodiments, p is 3. In some embodiments, o is 1. In some embodiments, o is 2.

In some embodiments, the compound of Formula (I-G) has the structure:

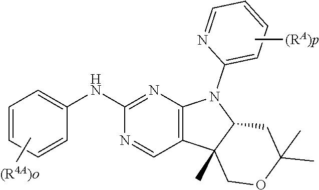

or a pharmaceutically acceptable salt thereof.

In some embodiments, the compound of Formula (I-G) has the structure:

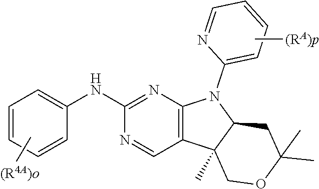

or a pharmaceutically acceptable salt thereof.

In some embodiments, the compound of Formula (I) is present in the form of a pharmaceutically acceptable salt. In some embodiments, the compound of Formula (I) is present in the form of a free base.

In some embodiments, the compound is selected from the group consisting of the compounds in Table 1, and pharmaceutically acceptable salts thereof.

TABLE 1
IUPAC Name
(4S)-4-methyl-3-(6-(4b,7,7-trimethyl-2-((4-(4-methylpiperazin-1-yl)
phenyl)amino)-4b,7,8,8a-tetrahydropyrano[3',4':4,5]pyrrolo[2,3-d]
pyrimidin-9(5H)-yl)pyridin-2-yl)oxazolidin-2-one
2-(6-(4b,7,7-trimethyl-2-((4-(4-methylpiperazin-1-yl)phenyl)amino)-
4b,7,8,8a-tetrahydropyrano[3',4':4,5]pyrrolo[2,3-d]pyrimidin-
9(5H)-yl)pyridin-2-yl)propan-2-ol
4b,7,7-trimethyl-9-(6-((methylamino)methyl)pyridin-2-yl)-N-(4-
morpholinophenyl)-4b,5,7,8,8a,9-hexahydropyrano[3',4':4,5]
pyrrolo[2,3-d]pyrimidin-2-amine
2-(6-(4b,7,7-trimethyl-2-((3-methyl-4-(1-methylpiperidin-4-yl)
phenyl)amino)-4b,7,8,8a-tetrahydropyrano[3',4':4,5]
pyrrolo[2,3-d]pyrimidin-9(5H)-yl)pyridin-2-yl)propan-2-ol
(4S)-4-methyl-3-(6-(4b,7,7-trimethyl-2-((1-(1-methylpiperidin-4-yl)-
1H-pyrazol-4-yl)amino)-4b,7,8,8a-tetrahydropyrano[3',4':4,5]
pyrrolo[2,3-d]pyrimidin-9(5H)-yl)pyridin-2-yl)oxazolidin-2-one
dimethyl((6-(4b,7,7-trimethyl-2-((4-(4-methylpiperazin-1-yl)phenyl)
amino)-4b,7,8,8a-tetrahydropyrano[3',4':4,5]
pyrrolo[2,3-d]pyrimidin-9(5H)-yl)pyridin-2-yl)imino)-I6-sulfanone
(4S)-4-methyl-3-(6-((8aR)-4b,7,7-trimethyl-2-((4-(5-methyl-2,5-
diazabicyclo[2.2.1]heptan-2-yl)phenyl)amino)-4b,7,8,8a-
tetrahydropyrano[3',4':4,5]pyrrolo[2,3-d]pyrimidin-9(5H)-yl)pyridin-2-
yl)oxazolidin-2-one
dimethyl((6-(4b,7,7-trimethyl-2-((1-(1-methylpiperidin-4-yl)-1H-
pyrazol-4-yl)amino)-4b,7,8,8a-tetrahydropyrano[3',4':4,5]
pyrrolo[2,3-d]pyrimidin-9(5H)-yl)pyridin-2-yl)imino)-I6-sulfanone
2-(6-(4b,7,7-trimethyl-2-((1-(1-methylpiperidin-4-yl)-1H-pyrazol-4-yl)
amino)-4b,7,8,8a-tetrahydropyrano[3',4':4,5]
pyrrolo[2,3-d]pyrimidin-9(5H)-yl)pyridin-2-yl)propan-2-ol
N-(4-(4,4-difluoropiperidin-1-yl)phenyl)-4b,7,7-trimethyl-
9-(6-((methylamino)methyl)pyridin-2-yl)-4b,5,7,8,8a,9-
hexahydropyrano[3',4':4,5]pyrrolo[2,3-d]pyrimidin-2-amine
4b,7,7-trimethyl-9-(6-((methylamino)methyl)pyridin-2-yl)-
N-(4-(tetrahydro-2H-pyran-4-yl)phenyl)-4b,5,7,8,8a,9-
hexahydropyrano[3',4':4,5]pyrrolo[2,3-d]pyrimidin-2-amine
4b,7,7-trimethyl-9-(6-((methylamino)methyl)pyridin-2-yl)-
N-(4-(tetrahydrofuran-3-yl)phenyl)-4b,5,7,8,8a,9-
hexahydropyrano[3',4':4,5]pyrrolo[2,3-d]pyrimidin-2-amine
dimethyl((6-(4b,7,7-trimethyl-2-((4-(1-methylpiperidin-4-yl)phenyl)
amino)-4b,7,8,8a-tetrahydropyrano[3',4':4,5]
pyrrolo[2,3-d]pyrimidin-9(5H)-yl)pyridin-2-yl)imino)-I6-sulfanone
1-methyl-5-(4-((4b,7,7-trimethyl-9-(6-((methylamino)methyl)pyridin-
2-yl)-4b,5,7,8,8a,9-hexahydropyrano[3',4':4,5]
pyrrolo[2,3-d]pyrimidin-2-yl)amino)phenyl)pyrrolidin-2-one
dimethyl((6-(4b,7,7-trimethyl-2-((2,4,4-trimethyl-1,2,3,4-
tetrahydroisoquinolin-7-yl)amino)-4b,7,8,8a-tetrahydropyrano[3',4':4,5]
pyrrolo[2,3-d]pyrimidin-9(5H)-yl)pyridin-2-yl)imino)-I6-sulfanone
dimethyl((6-((4bR)-4b,7,7-trimethyl-2-((3-methyl-4-(1-
methylpyrrolidin-3-yl)phenyl)amino)-4b,7,8,8a-tetrahydropyrano[3',4':
4,5]pyrrolo[2,3-d]pyrimidin-9(5H)-yl)pyridin-2-yl)imino)-I6-sulfanone
1-methyl-5-(5-((4b,7,7-trimethyl-9-(6-((methylamino)methyl)pyridin-
2-yl)-4b,5,7,8,8a,9-hexahydropyrano[3',4':4,5]
pyrrolo[2,3-d]pyrimidin-2-yl)amino)pyridin-2-yl)pyrrolidin-2-one
(4S)-4-methyl-3-(6-(4b,7,7-trimethyl-2-((6-(1-methylpyrrolidin-3-yl)
pyridin-3-yl)amino)-4b,7,8,8a-tetrahydropyrano[3',4':4,5]
pyrrolo[2,3-d]pyrimidin-9(5H)-yl)pyridin-2-yl)oxazolidin-2-one
(4S)-4-methyl-3-(6-(4b,7,7-trimethyl-2-((1-(1-methylpiperidin-4-yl)-2-
oxo-1,2-dihydropyridin-4-yl) amino)-4b,7,8,8a-tetrahydropyrano[3',4':
4,5]pyrrolo[2,3-d]pyrimidin-9(5H)-yl)pyridin-2-yl)oxazolidin-2-one
(4S)-4-methyl-3-(6-(4b,7,7-trimethyl-2-((6-(1-methylpiperidin-4-yl)
pyridin-3-yl)amino)-4b,7,8,8a-tetrahydropyrano[3',4':4,5]
pyrrolo[2,3-d]pyrimidin-9(5H)-yl)pyridin-2-yl)oxazolidin-2-one
(4S)-4-methyl-3-(6-(4b,7,7-trimethyl-2-((4-(1-methylpyrrolidin-3-yl)
phenyl)amino)-4b,7,8,8a-tetrahydropyrano[3',4':4,5]
pyrrolo[2,3-d]pyrimidin-9(5H)-yl)pyridin-2-yl)oxazolidin-2-one
(4S)-4-methyl-3-(6-(4b,7,7-trimethyl-2-((4-(1-methylazetidin-3-yl)
phenyl)amino)-4b,7,8,8a-tetrahydropyrano[3',4':4,5]
pyrrolo[2,3-d]pyrimidin-9(5H)-yl)pyridin-2-yl)oxazolidin-2-one
(4S)-4-methyl-3-(6-(4b,7,7-trimethyl-2-((4-((1-methylpyrrolidin-3-yl)
oxy)phenyl)amino)-4b,7,8,8a-tetrahydropyrano[3',4':4,5]
pyrrolo[2,3-d]pyrimidin-9(5H)-yl)pyridin-2-yl)oxazolidin-2-one
dimethyl((6-(4b,7,7-trimethyl-2-((6-(1-methylazetidin-3-yl)pyridin-3-yl)
amino)-4b,7,8,8a-tetrahydropyrano[3',4':4,5]
pyrrolo[2,3-d]pyrimidin-9(5H)-yl)pyridin-2-yl)imino)-I6-sulfanone
dimethyl((6-(4b,7,7-trimethyl-2-((1-(tetrahydro-2H-pyran-4-yl)-1H-
pyrazol-4-yl)amino)-4b,7,8,8a-tetrahydropyrano[3',4':4,5]
pyrrolo[2,3-d]pyrimidin-9(5H)-yl)pyridin-2-yl)imino)-I6-sulfanone
dimethyl((6-(4b,7,7-trimethyl-2-((1-(1-methylpyrrolidin-3-yl)-1H-
pyrazol-4-yl)amino)-4b,7,8,8a-tetrahydropyrano[3',4':4,5]
pyrrolo[2,3-d]pyrimidin-9(5H)-yl)pyridin-2-yl)imino)-I6-sulfanone
dimethyl((6-(4b,7,7-trimethyl-2-((6-(1-methylpyrrolidin-2-yl)pyridin-
3-yl)amino)-4b,7,8,8a-tetrahydropyrano[3',4':4,5]
pyrrolo[2,3-d]pyrimidin-9(5H)-yl)pyridin-2-yl)imino)-I6-sulfanone
dimethyl((6-(4b,7,7-trimethyl-2-((4-(6-methyl-2,6-diazaspiro[3.3]
heptan-2-yl)phenyl)amino)-4b,7,8,8a-tetrahydropyrano[3',4':4,5]
pyrrolo[2,3-d]pyrimidin-9(5H)-yl)pyridin-2-yl)imino)-I6-sulfanone
(4S)-4-methyl-3-(6-(4b,7,7-trimethyl-2-((4-((methylamino)methyl)
phenyl)amino)-4b,7,8,8a-tetrahydropyrano[3',4':4,5]
pyrrolo[2,3-d]pyrimidin-9(5H)-yl)pyridin-2-yl)oxazolidin-2-one
((6-(2-((4-(2,6-diazaspiro[3.3]heptan-2-yl)phenyl)amino)-4b,7,7-
trimethyl-4b,7,8,8a-tetrahydropyrano[3',4':4,5]pyrrolo[2,3-d]
pyrimidin-9(5H)-yl)pyridin-2-yl)imino)dimethyl-I6-sulfanone
((6-(2-((4-((S)-hexahydropyrrolo[1,2-a]pyrazin-2(1H)-yl)phenyl)
amino)-4b,7,7-trimethyl-4b,7,8,8a-tetrahydropyrano[3',4':4,5]
pyrrolo[2,3-d]pyrimidin-9(5H)-yl)pyridin-2-yl)imino)dimethyl-I6-
sulfanone
dimethyl((6-(4b,7,7-trimethyl-2-(pyridin-4-ylamino)-4b,7,8,8a-
tetrahydropyrano[3',4':4,5]pyrrolo[2,3-
d]pyrimidin-9(5H)-yl)pyridin-2-yl)imino)-I6-sulfanone
4b,7,7-trimethyl-9-(1-methyl-1H-pyrazol-3-yl)-N-(4-((1S,4S)-5-
methyl-2,5-diazabicyclo[2.2.1]heptan-2-yl)phenyl)-4b,5,7,8,8a,9-
hexahydropyrano[3',4':4,5]pyrrolo[2,3-d]pyrimidin-2-amine
4b,7,7-trimethyl-N-(4-morpholinophenyl)-9-(6-((R)-pyrrolidin-3-
yl)pyridin-2-yl)-4b,5,7,8,8a,9-hexahydropyrano[3',4':4,5]
pyrrolo[2,3-d]pyrimidin-2-amine
(S)-5-methyl-1-(6-(4b,7,7-trimethyl-2-((1-(1-methylpiperidin-4-yl)-
1H-pyrazol-4-yl)amino)-4b,7,8,8a-tetrahydropyrano[3',4':4,5]
pyrrolo[2,3-d]pyrimidin-9(5H)-yl)pyridin-2-yl)pyrrolidin-2-one
4b,7,7-trimethyl-9-((R)-7-methyl-7-(methylamino)-6,7-dihydro-
5H-cyclopenta[b]pyridin-2-yl)-N-(4-morpholinophenyl)-4b,5,7,8,8a,9-
hexahydropyrano[3',4':4,5]pyrrolo[2,3-d]pyrimidin-2-amine
4b-methyl-9-(6-((methylamino)methyl)pyridin-2-yl)-N-(4-
morpholinophenyl)-4b,5,7,8,8a,9-hexahydropyrano[3',4':4,5]
pyrrolo[2,3-d]pyrimidin-2-amine
N-(4-(2-oxa-5-azabicyclo[2.2.1]heptan-5-yl)phenyl)-4b,7,7-trimethyl-
9-(6-(1-(methylamino)ethyl)pyridin-2-yl)-4b,5,7,8,8a,9-
hexahydropyrano[3',4':4,5]pyrrolo[2,3-d]pyrimidin-2-amine
3-(6-(4b,7,7-trimethyl-2-((4-(pyrrolidin-3-yl)phenyl)amino)-4b,7,8,8a-
tetrahydropyrano[3',4':4,5]pyrrolo[2,3-d]pyrimidin-9(5H)-
yl)pyridin-2-yl)oxazolidin-2-one
1-(6-(4b,7,7-trimethyl-2-((4-((1-methylpyrrolidin-3-yl)oxy)phenyl)
amino)-4b,7,8,8a-tetrahydropyrano[3',4':4,5]pyrrolo[2,3-d]
pyrimidin-9(5H)-yl)pyridin-2-yl)pyrrolidin-2-one
1-(6-(4b,7,7-trimethyl-2-((3-methyl-4-((1S,4S)-5-methyl-2,5-
diazabicyclo[2.2.1]heptan-2-yl)phenyl)amino)-4b,7,8,8a-
tetrahydropyrano[3',4':4,5]pyrrolo[2,3-d]pyrimidin-9(5H)-yl)pyridin-
2-yl)pyrrolidin-2-one
(S)-4-methyl-3-(6-(4b-methyl-2-((4-(4-methylpiperazin-1-yl)phenyl)
amino)-4b,7,8,8a-tetrahydropyrano[3',4':4,5]
pyrrolo[2,3-d]pyrimidin-9(5H)-yl)pyridin-2-yl)oxazolidin-2-one
3-(6-(4b,7,7-trimethyl-2-((4-((3aR,6aR)-1-methylhexahydropyrrolo[3,4-
b]pyrrol-5(1H)-yl)phenyl)amino)-4b,7,8,8a-tetrahydropyrano[3',4':4,5]
pyrrolo[2,3-d]pyrimidin-9(5H)-yl)pyridin-2-yl)oxazolidin-2-one
dimethyl((3-(4b,7,7-trimethyl-2-((6-(1-methylpiperidin-4-yl)pyridin-3-yl)
amino)-4b,7,8,8a-tetrahydropyrano[3',4':4,5]pyrrolo[2,3-d]
pyrimidin-9(5H)-yl)phenyl)imino)-I6-sulfanone
dimethyl((3-(4b,7,7-trimethyl-2-((4-((1S,4S)-5-methyl-2,5-
diazabicyclo[2.2.1]heptan-2-yl)phenyl)amino)-4b,7,8,8a-
tetrahydropyrano[3',4':4,5]pyrrolo[2,3-d]pyrimidin-9(5H)-yl)phenyl)
imino)-I6-sulfanone
9-(1-isopropyl-1H-pyrazol-3-yl)-4b,7,7-trimethyl-N-(4-(4-
methylpiperazin-1-yl)phenyl)-4b,5,7,8,8a,9-hexahydropyrano[3',4':4,5]
pyrrolo[2,3-d]pyrimidin-2-amine
(S)-5-methyl-1-(6-(4b,7,7-trimethyl-2-((4-((1R,5S)-6-methyl-3,6-
diazabicyclo[3.1.1]heptan-3-yl)phenyl)amino)-4b,7,8,8a-
tetrahydropyrano[3',4':4,5]pyrrolo[2,3-d]pyrimidin-9(5H)-yl)pyridin-2-
yl)pyrrolidin-2-one
(R)-7-ethyl-2-(4b,7,7-trimethyl-2-((4-(4-methylpiperazin-1-yl)phenyl)
amino)-4b,7,8,8a-tetrahydropyrano[3',4':4,5]pyrrolo[2,3-d]
pyrimidin-9(5H)-yl)-6,7-dihydro-5H-cyclopenta[b]pyridin-7-ol
(S)-7-ethyl-2-(4b,7,7-trimethyl-2-((4-(4-methylpiperazin-1-yl)phenyl)
amino)-4b,7,8,8a-tetrahydropyrano[3',4':4,5]pyrrolo[2,3-d]
pyrimidin-9(5H)-yl)-6,7-dihydro-5H-cyclopenta[b]pyridin-7-ol
5,5-dimethyl-1-(6-(4b,7,7-trimethyl-2-((4-((1R,4R)-5-methyl-2,5-
diazabicyclo[2.2.1]heptan-2-yl)phenyl)amino)-4b,7,8,8a-
tetrahydropyrano[3',4':4,5]pyrrolo[2,3-d]pyrimidin-9(5H)-yl)pyridin-2-
yl)pyrrolidin-2-one
dimethyl((6-(4b,7,7-trimethyl-2-(pyrimidin-5-ylamino)-4b,7,8,8a-
tetrahydropyrano[3',4':4,5]pyrrolo[2,3-d]pyrimidin-9(5H)-yl)pyridin-
2-yl)imino)-I6-sulfanone

In some embodiments, the compound is selected from the group consisting of the compounds in Table 2, and pharmaceutically acceptable salts thereof. Absolute stereochemistry of the following examples was arbitrarily assigned.

TABLE 2

| Ex. # | IUPAC Name |
|---|---|
| 1, 2 | (4bS,8aS)-4b,7,7-trimethyl-9-(6-((methylamino)methyl)pyridin-2-yl)-N-(4-(piperidin-1- |
| | yl)phenyl)-4b,5,7,8,8a,9-hexahydropyrano[3′,4′:4,5]pyrrolo[2,3-d]pyrimidin-2-amine and |
| | (4bR,8aR)-4b,7,7-trimethyl-9-(6-((methylamino)methyl)pyridin-2-yl)-N-(4-(piperidin-1- |
| | yl)phenyl)-4b,5,7,8,8a,9-hexahydropyrano[3′,4′:4,5]pyrrolo[2,3-d]pyrimidin-2-amine |
| 3, 4 | 2-(6-((4bS,8aS)-4b,7,7-trimethyl-2-((4-(4-methylpiperazin-1-yl)phenyl)amino)-4b,7,8,8a- |
| | tetrahydropyrano[3′,4′:4,5]pyrrolo[2,3-d]pyrimidin-9(5H)-yl)pyridin-2-yl)propan-2-ol and 2-(6- |
| | ((4bR,8aR)-4b,7,7-trimethyl-2-((4-(4-methylpiperazin-1-yl)phenyl)amino)-4b,7,8,8a- |
| | tetrahydropyrano[3′,4′:4,5]pyrrolo[2,3-d]pyrimidin-9(5H)-yl)pyridin-2-yl)propan-2-ol |
| 5, 6 | (4bR,8aR)-4b,7,7-trimethyl-9-(6-((methylamino)methyl)pyridin-2-yl)-N-(4-morpholinophenyl)- |
| | 4b,5,7,8,8a,9-hexahydropyrano[3′,4′:4,5]pyrrolo[2,3-d]pyrimidin-2-amine and (4bS,8aS)- |
| | 4b,7,7-trimethyl-9-(6-((methylamino)methyl)pyridin-2-yl)-N-(4-morpholinophenyl)- |
| | 4b,5,7,8,8a,9-hexahydropyrano[3′,4′:4,5]pyrrolo[2,3-d]pyrimidin-2-amine |
| 7, 8 | 2-(6-((4bS,8aS)-4b,7,7-trimethyl-2-((3-methyl-4-(1-methylpiperidin-4-yl)phenyl)amino)- |
| | 4b,7,8,8a-tetrahydropyrano[3′,4′:4,5]pyrrolo[2,3-d]pyrimidin-9(5H)-yl)pyridin-2-yl)propan-2-ol |
| | and 2-(6-((4bR,8aR)-4b,7,7-trimethyl-2-((3-methyl-4-(1-methylpiperidin-4-yl)phenyl)amino)- |
| | 4b,7,8,8a-tetrahydropyrano[3′,4′:4,5]pyrrolo[2,3-d]pyrimidin-9(5H)-yl)pyridin-2-yl)propan-2-ol |
| 9, 10 | 2-(6-((4bR,8aR)-4b,7,7-trimethyl-2-((3-methyl-4-(4-methylpiperazin-1-yl)phenyl)amino)- |
| | 4b,7,8,8a-tetrahydropyrano[3′,4′:4,5]pyrrolo[2,3-d]pyrimidin-9(5H)-yl)pyridin-2-yl)propan-2-ol |
| | and 2-(6-((4bS,8aS)-4b,7,7-trimethyl-2-((3-methyl-4-(4-methylpiperazin-1-yl)phenyl)amino)- |
| | 4b,7,8,8a-tetrahydropyrano[3′,4′:4,5]pyrrolo[2,3-d]pyrimidin-9(5H)-yl)pyridin-2-yl)propan-2-ol |
| 11, 12 | 2-(6-((4bR,8aR)-4b,7,7-trimethyl-2-((1-(1-methylpiperidin-4-yl)-1H-pyrazol-4-yl)amino)- |
| | 4b,7,8,8a-tetrahydropyrano[3′,4′:4,5]pyrrolo[2,3-d]pyrimidin-9(5H)-yl)pyridin-2-yl)propan-2-ol |
| | and 2-(6-((4bS,8aS)-4b,7,7-trimethyl-2-((1-(1-methylpiperidin-4-yl)-1H-pyrazol-4-yl)amino)- |
| | 4b,7,8,8a-tetrahydropyrano[3′,4′:4,5]pyrrolo[2,3-d]pyrimidin-9(5H)-yl)pyridin-2-yl)propan-2-ol |
| 13, 14 | (4bS,8aS)-N-(3-chloro-4-morpholinophenyl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bR,8aR)-N-(3-chloro-4-morpholinophenyl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine |
| 15, 16 | (4bS,8aS)-N-(4-(4,4-difluoropiperidin-1-yl)phenyl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bR,8aR)-N-(4-(4,4-difluoropiperidin-1-yl)phenyl)-4b,7,7-trimethyl- |
| | 9-(6-((methylamino)methyl)pyridin-2-yl)-4b,5,7,8,8a,9-hexahydropyrano[3′,4′:4,5]pyrrolo[2,3- |
| | d]pyrimidin-2-amine |
| 17, 18 | (4bS,8aS)-N-(4-(1,4-oxazepan-4-yl)phenyl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bR,8aR)-N-(4-(1,4-oxazepan-4-yl)phenyl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine |
| 19, 20 | (4bS,8aS)-N-(4-((2S,6R)-2,6-dimethylmorpholino)phenyl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bR,8aR)-N-(4-((2S,6R)-2,6-dimethylmorpholino)phenyl)-4b,7,7- |
| | trimethyl-9-(6-((methylamino)methyl)pyridin-2-yl)-4b,5,7,8,8a,9- |
| | hexahydropyrano[3′,4′:4,5]pyrrolo[2,3-d]pyrimidin-2-amine |
| 21, 22 | (4bS,8aS)-N-(4-(3,3-difluoropyrrolidin-1-yl)phenyl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bR,8aR)-N-(4-(3,3-difluoropyrrolidin-1-yl)phenyl)-4b,7,7-trimethyl- |
| | 9-(6-((methylamino)methyl)pyridin-2-yl)-4b,5,7,8,8a,9-hexahydropyrano[3′,4′:4,5]pyrrolo[2,3- |
| | d]pyrimidin-2-amine |
| 23, 24 | morpholino(4-(((4bS,8aS)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2- |
| | yl)amino)phenyl)methanone and morpholino(4-(((4bR,8aR)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-yl)amino)phenyl)methanone |
| 25, 26 | (4bS,8aS)-4b,7,7-trimethyl-N-(3-methyl-4-morpholinophenyl)-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bR,8aR)-4b,7,7-trimethyl-N-(3-methyl-4-morpholinophenyl)-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine |
| 27, 28 | (S)-5-methyl-1-(4-(((4bS,8aS)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)phenyl)pyrrolidin- |
| | 2-one and (S)-5-methyl-1-(4-(((4bR,8aR)-4b,7,7-trimethyl-9-(6-((methylamino)methyl)pyridin- |
| | 2-yl)-4b,5,7,8,8a,9-hexahydropyrano[3′,4′:4,5]pyrrolo[2,3-d]pyrimidin-2- |
| | yl)amino)phenyl)pyrrolidin-2-one |
| 29, 30 | (R)-5-methyl-1-(4-(((4bS,8aS)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)phenyl)pyrrolidin- |
| | 2-one and (R)-5-methyl-1-(4-(((4bR,8aR)-4b,7,7-trimethyl-9-(6-((methylamino)methyl)pyridin- |
| | 2-yl)-4b,5,7,8,8a,9-hexahydropyrano[3′,4′:4,5]pyrrolo[2,3-d]pyrimidin-2- |
| | yl)amino)phenyl)pyrrolidin-2-one |
| 31, 32 | 2-methyl-6-(((4bS,8aS)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)isoindolin-1-one |
| | and 2-methyl-6-(((4bR,8aR)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)isoindolin-1-one |
| 33, 34 | (4bS,8aS)-4b,7,7-trimethyl-9-(6-((methylamino)methyl)pyridin-2-yl)-N-(4- |
| | (methylsulfonyl)phenyl)-4b,5,7,8,8a,9-hexahydropyrano[3′,4′:4,5]pyrrolo[2,3-d]pyrimidin-2- |
| | amine and (4bR,8aR)-4b,7,7-trimethyl-9-(6-((methylamino)methyl)pyridin-2-yl)-N-(4- |
| | (methylsulfonyl)phenyl)-4b,5,7,8,8a,9-hexahydropyrano[3′,4′:4,5]pyrrolo[2,3-d]pyrimidin-2- |
| | amine |
| 35, 36 | (4bR,8aR)-4b,7,7-trimethyl-9-(6-((methylamino)methyl)pyridin-2-yl)-N-(4-(tetrahydro-2H- |
| | pyran-4-yl)phenyl)-4b,5,7,8,8a,9-hexahydropyrano[3′,4′:4,5]pyrrolo[2,3-d]pyrimidin-2-amine |
| | and (4bS,8aS)-4b,7,7-trimethyl-9-(6-((methylamino)methyl)pyridin-2-yl)-N-(4-(tetrahydro-2H- |
| | pyran-4-yl)phenyl)-4b,5,7,8,8a,9-hexahydropyrano[3′,4′:4,5]pyrrolo[2,3-d]pyrimidin-2-amine |
| 37, 38 | (4bS,8aS)-4b,7,7-trimethyl-9-(6-((methylamino)methyl)pyridin-2-yl)-N-(4-((S)-tetrahydrofuran- |
| | 3-yl)phenyl)-4b,5,7,8,8a,9-hexahydropyrano[3′,4′:4,5]pyrrolo[2,3-d]pyrimidin-2-amine and |
| | (4bR,8aR)-4b,7,7-trimethyl-9-(6-((methylamino)methyl)pyridin-2-yl)-N-(4-((S)-tetrahydrofuran- |
| | 3-yl)phenyl)-4b,5,7,8,8a,9-hexahydropyrano[3′,4′:4,5]pyrrolo[2,3-d]pyrimidin-2-amine |
| 39, 40 | (4bS,8aS)-4b,7,7-trimethyl-9-(6-((methylamino)methyl)pyridin-2-yl)-N-(4-((R)-tetrahydrofuran- |
| | 3-yl)phenyl)-4b,5,7,8,8a,9-hexahydropyrano[3′,4′:4,5]pyrrolo[2,3-d]pyrimidin-2-amine and |
| | (4bR,8aR)-4b,7,7-trimethyl-9-(6-((methylamino)methyl)pyridin-2-yl)-N-(4-((R)- |
| | tetrahydrofuran-3-yl)phenyl)-4b,5,7,8,8a,9-hexahydropyrano[3′,4′:4,5]pyrrolo[2,3-d]pyrimidin- |
| | 2-amine |
| 41, 42 | (4bR,8aR)-N-(4-((1R,4R)-2-oxa-5-azabicyclo[2.2.1]heptan-5-yl)phenyl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bS,8aS)-N-(4-((1R,4R)-2-oxa-5-azabicyclo[2.2.1]heptan-5- |
| | yl)phenyl)-4b,7,7-trimethyl-9-(6-((methylamino)methyl)pyridin-2-yl)-4b,5,7,8,8a,9- |
| | hexahydropyrano[3′,4′:4,5]pyrrolo[2,3-d]pyrimidin-2-amine |
| 43, 44 | (4bS,8aS)-N-(4-((1R,4R)-2-oxa-5-azabicyclo[2.2.1]heptan-5-yl)phenyl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bR,8aR)-N-(4-((1R,4R)-2-oxa-5-azabicyclo[2.2.1]heptan-5- |
| | yl)phenyl)-4b,7,7-trimethyl-9-(6-((methylamino)methyl)pyridin-2-yl)-4b,5,7,8,8a,9- |
| | hexahydropyrano[3′,4′:4,5]pyrrolo[2,3-d]pyrimidin-2-amine |
| 45, 46 | (4bR,8aR)-4b,7,7-trimethyl-9-(6-((methylamino)methyl)pyridin-2-yl)-N-phenyl-4b,5,7,8,8a,9- |
| | hexahydropyrano[3′,4′:4,5]pyrrolo[2,3-d]pyrimidin-2-amine and (4bS,8aS)-4b,7,7-trimethyl-9- |
| | (6-((methylamino)methyl)pyridin-2-yl)-N-phenyl-4b,5,7,8,8a,9- |
| | hexahydropyrano[3′,4′:4,5]pyrrolo[2,3-d]pyrimidin-2-amine |
| 47, 48 | (4bS,8aS)-N-(1-(tert-butyl)-1H-pyrazol-4-yl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bR,8aR)-N-(1-(tert-butyl)-1H-pyrazol-4-yl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine |
| 49, 50 | 1-imino-4-(4-(((4bR,8aR)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)phenyl)-116- |
| | thiomorpholine 1-oxide and 1-imino-4-(4-(((4bS,8aS)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-yl)amino)phenyl)-116-thiomorpholine 1-oxide |
| 51, 52 | (S)-1-methyl-5-(4-(((4bS,8aS)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)phenyl)pyrrolidin- |
| | 2-one and (S)-1-methyl-5-(4-(((4bR,8aR)-4b,7,7-trimethyl-9-(6-((methylamino)methyl)pyridin- |
| | 2-yl)-4b,5,7,8,8a,9-hexahydropyrano[3′,4′:4,5]pyrrolo[2,3-d]pyrimidin-2- |
| | yl)amino)phenyl)pyrrolidin-2-one |
| 53, 54 | (R)-1-methyl-5-(4-(((4bS,8aS)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)phenyl)pyrrolidin- |
| | 2-one and (R)-1-methyl-5-(4-(((4bR,8aR)-4b,7,7-trimethyl-9-(6-((methylamino)methyl)pyridin- |
| | 2-yl)-4b,5,7,8,8a,9-hexahydropyrano[3′,4′:4,5]pyrrolo[2,3-d]pyrimidin-2- |
| | yl)amino)phenyl)pyrrolidin-2-one |
| 55, 56 | (4bS,8aS)-4b,7,7-trimethyl-9-(6-((methylamino)methyl)pyridin-2-yl)-N-(6-(tetrahydro-2H- |
| | pyran-4-yl)pyridin-3-yl)-4b,5,7,8,8a,9-hexahydropyrano[3′,4′:4,5]pyrrolo[2,3-d]pyrimidin-2- |
| | amine and (4bR,8aR)-4b,7,7-trimethyl-9-(6-((methylamino)methyl)pyridin-2-yl)-N-(6- |
| | (tetrahydro-2H-pyran-4-yl)pyridin-3-yl)-4b,5,7,8,8a,9-hexahydropyrano[3′,4′:4,5]pyrrolo[2,3- |
| | d]pyrimidin-2-amine |
| 57, 58 | (4bS,8aS)-4b,7,7-trimethyl-9-(6-((methylamino)methyl)pyridin-2-yl)-N-(4-((S)-tetrahydrofuran- |
| | 2-yl)phenyl)-4b,5,7,8,8a,9-hexahydropyrano[3′,4′:4,5]pyrrolo[2,3-d]pyrimidin-2-amine and |
| | (4bS,8aS)-4b,7,7-trimethyl-9-(6-((methylamino)methyl)pyridin-2-yl)-N-(4-((R)-tetrahydrofuran- |
| | 2-yl)phenyl)-4b,5,7,8,8a,9-hexahydropyrano[3′,4′:4,5]pyrrolo[2,3-d]pyrimidin-2-amine |
| 59, 60 | (4bR,8aR)-4b,7,7-trimethyl-9-(6-((methylamino)methyl)pyridin-2-yl)-N-(4-((S)-tetrahydrofuran- |
| | 2-yl)phenyl)-4b,5,7,8,8a,9-hexahydropyrano[3′,4′:4,5]pyrrolo[2,3-d]pyrimidin-2-amine and |
| | (4bR,8aR)-4b,7,7-trimethyl-9-(6-((methylamino)methyl)pyridin-2-yl)-N-(4-((R)- |
| | tetrahydrofuran-2-yl)phenyl)-4b,5,7,8,8a,9-hexahydropyrano[3′,4′:4,5]pyrrolo[2,3-d]pyrimidin- |
| | 2-amine |
| 61, 62 | (S)-4-(4-(((4bR,8aR)-4b,7,7-trimethyl-9-(6-((methylamino)methyl)pyridin-2-yl)-4b,5,7,8,8a,9- |
| | hexahydropyrano[3′,4′:4,5]pyrrolo[2,3-d]pyrimidin-2-yl)amino)phenyl)pyrrolidin-2-one and (R)- |
| | 4-(4-(((4bR,8aR)-4b,7,7-trimethyl-9-(6-((methylamino)methyl)pyridin-2-yl)-4b,5,7,8,8a,9- |
| | hexahydropyrano[3′,4′:4,5]pyrrolo[2,3-d]pyrimidin-2-yl)amino)phenyl)pyrrolidin-2-one |
| 63, 64 | (R)-4-(4-(((4bR,8aR)-4b,7,7-trimethyl-9-(6-((methylamino)methyl)pyridin-2-yl)-4b,5,7,8,8a,9- |
| | hexahydropyrano[3′,4′:4,5]pyrrolo[2,3-d]pyrimidin-2-yl)amino)phenyl)pyrrolidin-2-one and (R)- |
| | 4-(4-(((4bS,8aS)-4b,7,7-trimethyl-9-(6-((methylamino)methyl)pyridin-2-yl)-4b,5,7,8,8a,9- |
| | hexahydropyrano[3′,4′:4,5]pyrrolo[2,3-d]pyrimidin-2-yl)amino)phenyl)pyrrolidin-2-one |
| 65, 66 | 1-(methylimino)-4-(4-(((4bR,8aR)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)phenyl)-116- |
| | thiomorpholine 1-oxide and 1-(methylimino)-4-(4-(((4bS,8aS)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-yl)amino)phenyl)-116-thiomorpholine 1-oxide |
| 67, 68 | (R)-1-methyl-4-(4-(((4bS,8aS)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)phenyl)pyrrolidin- |
| | 2-one and (R)-1-methyl-4-(4-(((4bR,8aR)-4b,7,7-trimethyl-9-(6-((methylamino)methyl)pyridin- |
| | 2-yl)-4b,5,7,8,8a,9-hexahydropyrano[3′,4′:4,5]pyrrolo[2,3-d]pyrimidin-2- |
| | yl)amino)phenyl)pyrrolidin-2-one |
| 69, 70 | (S)-1-methyl-4-(4-(((4bR,8aR)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)phenyl)pyrrolidin- |
| | 2-one and (S)-1-methyl-4-(4-(((4bS,8aS)-4b,7,7-trimethyl-9-(6-((methylamino)methyl)pyridin- |
| | 2-yl)-4b,5,7,8,8a,9-hexahydropyrano[3′,4′:4,5]pyrrolo[2,3-d]pyrimidin-2- |
| | yl)amino)phenyl)pyrrolidin-2-one |
| 71, 72 | (4bS,8aS)-4b,7,7-trimethyl-N-(3-methyl-4-(1-methylpiperidin-4-yl)phenyl)-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bR,8aR)-4b,7,7-trimethyl-N-(3-methyl-4-(1-methylpiperidin-4- |
| | yl)phenyl)-9-(6-((methylamino)methyl)pyridin-2-yl)-4b,5,7,8,8a,9- |
| | hexahydropyrano[3′,4′:4,5]pyrrolo[2,3-d]pyrimidin-2-amine |
| 73, 74 | (4bR,8aR)-N-(4-(tert-butyl)phenyl)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-amine and (4bS,8aS)-N-(4- |
| | (tert-butyl)phenyl)-4b,7,7-trimethyl-9-(6-((methylamino)methyl)pyridin-2-yl)-4b,5,7,8,8a,9- |
| | hexahydropyrano[3′,4′:4,5]pyrrolo[2,3-d]pyrimidin-2-amine |
| 75, 76 | N,N,2-trimethyl-2-(4-(((4bS,8aS)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2- |
| | yl)amino)phenyl)propanamide and N,N,2-trimethyl-2-(4-(((4bR,8aR)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-yl)amino)phenyl)propanamide |
| 77, 78 | (S)-1,3-dimethyl-3-(4-(((4bS,8aS)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)phenyl)pyrrolidin- |
| | 2-one and (S)-1,3-dimethyl-3-(4-(((4bR,8aR)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-yl)amino)phenyl)pyrrolidin-2-one |
| 79, 80 | (R)-1,3-dimethyl-3-(4-(((4bS,8aS)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)phenyl)pyrrolidin- |
| | 2-one and (R)-1,3-dimethyl-3-(4-(((4bR,8aR)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-yl)amino)phenyl)pyrrolidin-2-one |
| 81, 82 | (4bR,8aR)-4b,7,7-trimethyl-9-(6-((methylamino)methyl)pyridin-2-yl)-N-(4-(3-methyloxetan-3- |
| | yl)phenyl)-4b,5,7,8,8a,9-hexahydropyrano[3′,4′:4,5]pyrrolo[2,3-d]pyrimidin-2-amine and |
| | (4bS,8aS)-4b,7,7-trimethyl-9-(6-((methylamino)methyl)pyridin-2-yl)-N-(4-(3-methyloxetan-3- |
| | yl)phenyl)-4b,5,7,8,8a,9-hexahydropyrano[3′,4′:4,5]pyrrolo[2,3-d]pyrimidin-2-amine |
| 83, 84 | (4bS,8aS)-4b,7,7-trimethyl-9-(6-((methylamino)methyl)pyridin-2-yl)-N-(4-(3-methyloxetan-3- |
| | yl)phenyl)-4b,5,7,8,8a,9-hexahydropyrano[3′,4′:4,5]pyrrolo[2,3-d]pyrimidin-2-amine and |
| | (4bR,8aR)-4b,7,7-trimethyl-9-(6-((methylamino)methyl)pyridin-2-yl)-N-(4-(3-methyloxetan-3- |
| | yl)phenyl)-4b,5,7,8,8a,9-hexahydropyrano[3′,4′:4,5]pyrrolo[2,3-d]pyrimidin-2-amine |
| 85, 86 | (4bR,8aR)-N-(4-(3-oxa-8-azabicyclo[3.2.1]octan-8-yl)-3-methylphenyl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bS,8aS)-N-(4-(3-oxa-8-azabicyclo[3.2.1]octan-8-yl)-3- |
| | methylphenyl)-4b,7,7-trimethyl-9-(6-((methylamino)methyl)pyridin-2-yl)-4b,5,7,8,8a,9- |
| | hexahydropyrano[3′,4′:4,5]pyrrolo[2,3-d]pyrimidin-2-amine |
| 87, 88 | (4bR,8aR)-N-(4-(3-oxa-8-azabicyclo[3.2.1]octan-8-yl)phenyl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bS,8aS)-N-(4-(3-oxa-8-azabicyclo[3.2.1]octan-8-yl)phenyl)-4b,7,7- |
| | trimethyl-9-(6-((methylamino)methyl)pyridin-2-yl)-4b,5,7,8,8a,9- |
| | hexahydropyrano[3′,4′:4,5]pyrrolo[2,3-d]pyrimidin-2-amine |
| 89, 90 | (4bR,8aR)-N-(bicyclo[1.1.1]pentan-1-yl)-4b,7,7-trimethyl-9-(6-((methylamino)methyl)pyridin- |
| | 2-yl)-4b,5,7,8,8a,9-hexahydropyrano[3′,4′:4,5]pyrrolo[2,3-d]pyrimidin-2-amine and (4bS,8aS)- |
| | N-(bicyclo[1.1.1]pentan-1-yl)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-amine |
| 91, 92 | 2,4,4-trimethyl-7-(((4bS,8aS)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)-3,4- |
| | dihydroisoquinolin-1(2H)-one and 2,4,4-trimethyl-7-(((4bR,8aR)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-yl)amino)-3,4-dihydroisoquinolin-1(2H)-one |
| 93, 94 | (4bS,8aS)-4b,7,7-trimethyl-9-(6-((methylamino)methyl)pyridin-2-yl)-N-(4-(4-methylpiperazin- |
| | 1-yl)phenyl)-4b,5,7,8,8a,9-hexahydropyrano[3′,4′:4,5]pyrrolo[2,3-d]pyrimidin-2-amine and |
| | (4bR,8aR)-4b,7,7-trimethyl-9-(6-((methylamino)methyl)pyridin-2-yl)-N-(4-(4-methylpiperazin- |
| | 1-yl)phenyl)-4b,5,7,8,8a,9-hexahydropyrano[3′,4′:4,5]pyrrolo[2,3-d]pyrimidin-2-amine |
| 95, 96 | (S)-1-methyl-5-(5-(((4bS,8aS)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)pyridin-2- |
| | yl)pyrrolidin-2-one and (S)-1-methyl-5-(5-(((4bR,8aR)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-yl)amino)pyridin-2-yl)pyrrolidin-2-one |
| 97, 98 | (R)-1-methyl-5-(5-(((4bS,8aS)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)pyridin-2- |
| | yl)pyrrolidin-2-one and (R)-1-methyl-5-(5-(((4bR,8aR)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-yl)amino)pyridin-2-yl)pyrrolidin-2-one |
| 99, 100 | dimethyl((6-((4bS,8aS)-4b,7,7-trimethyl-2-((4-(4-methylpiperazin-1-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bR,8aR)-4b,7,7-trimethyl-2-((4-(4-methylpiperazin-1- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone |
| 101, 102 | dimethyl((6-((4bS,8aS)-4b,7,7-trimethyl-2-((1-(1-methylpiperidin-4-yl)-1H-pyrazol-4-yl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bR,8aR)-4b,7,7-trimethyl-2-((1-(1-methylpiperidin-4-yl)-1H- |
| | pyrazol-4-yl)amino)-4b,7,8,8a-tetrahydropyrano[3′,4′:4,5]pyrrolo[2,3-d]pyrimidin-9(5H)- |
| | yl)pyridin-2-yl)imino)-16-sulfanone |
| 103, 104 | dimethyl((6-((4bS,8aS)-4b,7,7-trimethyl-2-((3-methyl-4-(1-methylpiperidin-4-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bR,8aR)-4b,7,7-trimethyl-2-((3-methyl-4-(1-methylpiperidin-4- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone |
| 105, 106 | dimethyl((6-((4bS,8aS)-4b,7,7-trimethyl-2-((3-methyl-4-(4-methylpiperazin-1- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone and dimethyl((6-((4bR,8aR)-4b,7,7-trimethyl-2-((3-methyl-4-(4- |
| | methylpiperazin-1-yl)phenyl)amino)-4b,7,8,8a-tetrahydropyrano[3′,4′:4,5]pyrrolo[2,3- |
| | d]pyrimidin-9(5H)-yl)pyridin-2-yl)imino)-16-sulfanone |
| 107, 108 | dimethyl((6-((4bR,8aR)-4b,7,7-trimethyl-2-((2′-methyl-2′,3′-dihydro-1′H-spiro[cyclopropane- |
| | 1,4′-isoquinolin]-7′-yl)amino)-4b,7,8,8a-tetrahydropyrano[3′,4′:4,5]pyrrolo[2,3-d]pyrimidin- |
| | 9(5H)-yl)pyridin-2-yl)imino)-16-sulfanone and dimethyl((6-((4bS,8aS)-4b,7,7-trimethyl-2-((2′- |
| | methyl-2′,3′-dihydro-1′H-spiro[cyclopropane-1,4′-isoquinolin]-7′-yl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 109, 110 | dimethyl((6-((4bR,8aR)-4b,7,7-trimethyl-2-((2-methyl-1,2,3,4-tetrahydroisoquinolin-7- |
| | yl)amino)-4b,7,8,8a-tetrahydropyrano[3′,4′:4,5]pyrrolo[2,3-d]pyrimidin-9(5H)-yl)pyridin-2- |
| | yl)imino)-16-sulfanone and dimethyl((6-((4bS,8aS)-4b,7,7-trimethyl-2-((2-methyl-1,2,3,4- |
| | tetrahydroisoquinolin-7-yl)amino)-4b,7,8,8a-tetrahydropyrano[3′,4′:4,5]pyrrolo[2,3-d]pyrimidin- |
| | 9(5H)-yl)pyridin-2-yl)imino)-16-sulfanone |
| | dimethyl((6-((4bS,8aS)-4b,7,7-trimethyl-2-((4-(1-methylpiperidin-4-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| 113, 114 | dimethyl((6-((4bS,8aS)-4b,7,7-trimethyl-2-((3-methyl-4-((R)-morpholin-3-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bR,8aR)-4b,7,7-trimethyl-2-((3-methyl-4-((R)-morpholin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone |
| 115, 120 | dimethyl((6-((4bS,8aS)-4b,7,7-trimethyl-2-((3-methyl-4-((S)-morpholin-3-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bR,8aR)-4b,7,7-trimethyl-2-((3-methyl-4-((S)-morpholin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone |
| 116, 117 | 4-(((4bS,8aS)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)amino)-N- |
| | methylbenzamide and 4-(((4bR,8aR)-9-(6-((dimethyl(oxo)-16-sulfaneylidene)amino)pyridin-2- |
| | yl)-4b,7,7-trimethyl-4b,5,7,8,8a,9-hexahydropyrano[3′,4′:4,5]pyrrolo[2,3-d]pyrimidin-2- |
| | yl)amino)-N-methylbenzamide |
| 118, 119 | 4-(((4bS,8aS)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)amino)-2-fluoro-N- |
| | methylbenzamide and 4-(((4bR,8aR)-9-(6-((dimethyl(oxo)-16-sulfaneylidene)amino)pyridin-2- |
| | yl)-4b,7,7-trimethyl-4b,5,7,8,8a,9-hexahydropyrano[3′,4′:4,5]pyrrolo[2,3-d]pyrimidin-2- |
| | yl)amino)-2-fluoro-N-methylbenzamide |
| 121, 122 | dimethyl((6-((4bS,8aS)-4b,7,7-trimethyl-2-((4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| | and dimethyl((6-((4bR,8aR)-4b,7,7-trimethyl-2-((4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 123, 124 | dimethyl((6-((4bS,8aS)-4b,7,7-trimethyl-2-((4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| | and dimethyl((6-((4bR,8aR)-4b,7,7-trimethyl-2-((4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 125, 126 | dimethyl((6-((4bS,8aS)-4b,7,7-trimethyl-2-((4-((S)-1-methylpyrrolidin-2-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bR,8aR)-4b,7,7-trimethyl-2-((4-((S)-1-methylpyrrolidin-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone |
| 127, 128 | dimethyl((6-((4bR,8aR)-4b,7,7-trimethyl-2-((4-((R)-1-methylpyrrolidin-2-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bS,8aS)-4b,7,7-trimethyl-2-((4-((R)-1-methylpyrrolidin-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone |
| 129, 130 | dimethyl((6-((4bR,8aR)-4b,7,7-trimethyl-2-((4-((S)-1-methylpyrrolidin-3-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bS,8aS)-4b,7,7-trimethyl-2-((4-((S)-1-methylpyrrolidin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone |
| 131, 132 | dimethyl((6-((4bR,8aR)-4b,7,7-trimethyl-2-((4-((S)-pyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| | and dimethyl((6-((4bS,8aS)-4b,7,7-trimethyl-2-((4-((S)-pyrrolidin-3-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone |
| 133, 134 | dimethyl((6-((4bR,8aR)-4b,7,7-trimethyl-2-((4-((R)-1-methylpyrrolidin-3-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bS,8aS)-4b,7,7-trimethyl-2-((4-((R)-1-methylpyrrolidin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone |
| 135, 136 | dimethyl((6-((4bR,8aR)-4b,7,7-trimethyl-2-((2,4,4-trimethyl-1,2,3,4-tetrahydroisoquinolin-7- |
| | yl)amino)-4b,7,8,8a-tetrahydropyrano[3′,4′:4,5]pyrrolo[2,3-d]pyrimidin-9(5H)-yl)pyridin-2- |
| | yl)imino)-16-sulfanone and dimethyl((6-((4bS,8aS)-4b,7,7-trimethyl-2-((2,4,4-trimethyl-1,2,3,4- |
| | tetrahydroisoquinolin-7-yl)amino)-4b,7,8,8a-tetrahydropyrano[3′,4′:4,5]pyrrolo[2,3-d]pyrimidin- |
| | 9(5H)-yl)pyridin-2-yl)imino)-16-sulfanone |
| 137, 138 | dimethyl((6-((4bR,8aR)-4b,7,7-trimethyl-2-((4-((R)-pyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| | and dimethyl((6-((4bS,8aS)-4b,7,7-trimethyl-2-((4-((R)-pyrrolidin-3-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone |
| 139, 140 | dimethyl((6-((4bS,8aS)-4b,7,7-trimethyl-2-((6-(1-methylpiperidin-4-yl)pyridin-3-yl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bR,8aR)-4b,7,7-trimethyl-2-((6-(1-methylpiperidin-4-yl)pyridin-3- |
| | yl)amino)-4b,7,8,8a-tetrahydropyrano[3′,4′:4,5]pyrrolo[2,3-d]pyrimidin-9(5H)-yl)pyridin-2- |
| | yl)imino)-16-sulfanone |
| 141, 142 | 4-(((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)amino)-N,N- |
| | dimethylbenzamide and 4-(((4bS,8aS)-9-(6-((dimethyl(oxo)-16-sulfaneylidene)amino)pyridin- |
| | 2-yl)-4b,7,7-trimethyl-4b,5,7,8,8a,9-hexahydropyrano[3′,4′:4,5]pyrrolo[2,3-d]pyrimidin-2- |
| | yl)amino)-N,N-dimethylbenzamide |
| 143, 144 | dimethyl((6-((4bR,8aR)-4b,7,7-trimethyl-2-((3-methyl-4-((S)-pyrrolidin-3-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bS,8aS)-4b,7,7-trimethyl-2-((3-methyl-4-((S)-pyrrolidin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone |
| 145, 146 | dimethyl((6-((4bR,8aR)-4b,7,7-trimethyl-2-((3-methyl-4-((S)-1-methylpyrrolidin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone and dimethyl((6-((4bS,8aS)-4b,7,7-trimethyl-2-((3-methyl-4-((S)-1- |
| | methylpyrrolidin-3-yl)phenyl)amino)-4b,7,8,8a-tetrahydropyrano[3′,4′:4,5]pyrrolo[2,3- |
| | d]pyrimidin-9(5H)-yl)pyridin-2-yl)imino)-16-sulfanone |
| 147, 148 | dimethyl((6-((4bR,8aR)-4b,7,7-trimethyl-2-((3-methyl-4-((R)-1-methylpyrrolidin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone and dimethyl((6-((4bS,8aS)-4b,7,7-trimethyl-2-((3-methyl-4-((R)-1- |
| | methylpyrrolidin-3-yl)phenyl)amino)-4b,7,8,8a-tetrahydropyrano[3′,4′:4,5]pyrrolo[2,3- |
| | d]pyrimidin-9(5H)-yl)pyridin-2-yl)imino)-16-sulfanone |
| 149, 150 | dimethyl((6-((4bR,8aR)-4b,7,7-trimethyl-2-((3-methyl-4-((R)-pyrrolidin-3-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bS,8aS)-4b,7,7-trimethyl-2-((3-methyl-4-((R)-pyrrolidin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone |
| 151, 152 | 4-(((4bS,8aS)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)amino)-1-(1- |
| | methylpiperidin-4-yl)pyridin-2(1H)-one and 4-(((4bR,8aR)-9-(6-((dimethyl(oxo)-16- |
| | sulfaneylidene)amino)pyridin-2-yl)-4b,7,7-trimethyl-4b,5,7,8,8a,9- |
| | hexahydropyrano[3′,4′:4,5]pyrrolo[2,3-d]pyrimidin-2-yl)amino)-1-(1-methylpiperidin-4- |
| | yl)pyridin-2(1H)-one |
| 153, 154 | dimethyl((6-((4bR,8aR)-4b,7,7-trimethyl-2-((4-(((S)-pyrrolidin-3-yl)oxy)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bS,8aS)-4b,7,7-trimethyl-2-((4-(((S)-pyrrolidin-3- |
| | yl)oxy)phenyl)amino)-4b,7,8,8a-tetrahydropyrano[3′,4′:4,5]pyrrolo[2,3-d]pyrimidin-9(5H)- |
| | yl)pyridin-2-yl)imino)-16-sulfanone |
| 155, 156 | dimethyl((6-((4bR,8aR)-4b,7,7-trimethyl-2-((4-(((R)-pyrrolidin-3-yl)oxy)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bS,8aS)-4b,7,7-trimethyl-2-((4-(((R)-pyrrolidin-3- |
| | yl)oxy)phenyl)amino)-4b,7,8,8a-tetrahydropyrano[3′,4′:4,5]pyrrolo[2,3-d]pyrimidin-9(5H)- |
| | yl)pyridin-2-yl)imino)-16-sulfanone |
| 157, 158 | dimethyl((6-((4bR,8aR)-4b,7,7-trimethyl-2-((4-(((S)-1-methylpyrrolidin-3- |
| | yl)oxy)phenyl)amino)-4b,7,8,8a-tetrahydropyrano[3′,4′:4,5]pyrrolo[2,3-d]pyrimidin-9(5H)- |
| | yl)pyridin-2-yl)imino)-16-sulfanone and dimethyl((6-((4bS,8aS)-4b,7,7-trimethyl-2-((4-(((S)-1- |
| | methylpyrrolidin-3-yl)oxy)phenyl)amino)-4b,7,8,8a-tetrahydropyrano[3′,4′:4,5]pyrrolo[2,3- |
| | d]pyrimidin-9(5H)-yl)pyridin-2-yl)imino)-16-sulfanone |
| 159, 160 | dimethyl((6-((4bR,8aR)-4b,7,7-trimethyl-2-((4-(((R)-1-methylpyrrolidin-3- |
| | yl)oxy)phenyl)amino)-4b,7,8,8a-tetrahydropyrano[3′,4′:4,5]pyrrolo[2,3-d]pyrimidin-9(5H)- |
| | yl)pyridin-2-yl)imino)-16-sulfanone and dimethyl((6-((4bS,8aS)-4b,7,7-trimethyl-2-((4-(((R)-1- |
| | methylpyrrolidin-3-yl)oxy)phenyl)amino)-4b,7,8,8a-tetrahydropyrano[3′,4′:4,5]pyrrolo[2,3- |
| | d]pyrimidin-9(5H)-yl)pyridin-2-yl)imino)-16-sulfanone |
| 161, 162 | dimethyl((6-((4bS,8aS)-4b,7,7-trimethyl-2-((4-(1-(methyl-d3)piperidin-4-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bR,8aR)-4b,7,7-trimethyl-2-((4-(1-(methyl-d3)piperidin-4- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone |
| 163, 164 | ((6-((4bR,8aR)-2-((4-(azetidin-3-yl)phenyl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone and ((6-((4bS,8aS)-2-((4-(azetidin-3-yl)phenyl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 165, 166 | dimethyl((6-((4bR,8aR)-4b,7,7-trimethyl-2-((4-(1-methylazetidin-3-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bS,8aS)-4b,7,7-trimethyl-2-((4-(1-methylazetidin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone |
| 167, 168 | ((6-((4bS,8aS)-2-((4-((R)-1,3-dimethylpyrrolidin-3-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2- |
| | yl)imino) dimethyl-16-sulfanone and ((6-((4bR,8aR)-2-((4-((R)-1,3-dimethylpyrrolidin-3- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)imino)dimethyl-16-sulfanone |
| 169, 170 | ((6-((4bR,8aR)-2-((4-((S)-1,3-dimethylpyrrolidin-3-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2- |
| | yl)imino)dimethyl-16-sulfanone and ((6-((4bS,8aS)-2-((4-((S)-1,3-dimethylpyrrolidin-3- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)imino)dimethyl-16-sulfanone |
| 171, 172 | dimethyl((6-((4bR,8aR)-4b,7,7-trimethyl-2-((4-((S)-3-methylpyrrolidin-3-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bS,8aS)-4b,7,7-trimethyl-2-((4-((S)-3-methylpyrrolidin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone |
| 173, 174 | dimethyl((6-((4bR,8aR)-4b,7,7-trimethyl-2-((4-((R)-3-methylpyrrolidin-3-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bS,8aS)-4b,7,7-trimethyl-2-((4-((R)-3-methylpyrrolidin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone |
| 175, 176 | ((6-((4bS,8aS)-2-((3-fluoro-4-((1R,4R)-5-methyl-2,5-diazabicyclo[2.2.1]heptan-2- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)imino)dimethyl-16-sulfanone and ((6-((4bR,8aR)-2-((3-fluoro- |
| | 4-((1R,4R)-5-methyl-2,5-diazabicyclo[2.2.1]heptan-2-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2- |
| | yl)imino)dimethyl-16-sulfanone |
| 177, 178 | ((6-((4bR,8aR)-2-((3-fluoro-4-(1-methylpiperidin-4-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2- |
| | yl)imino)dimethyl-16-sulfanone and ((6-((4bS,8aS)-2-((3-fluoro-4-(1-methylpiperidin-4- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)imino)dimethyl-16-sulfanone |
| 179, 180 | ((6-((4bS,8aS)-2-((3-fluoro-4-((1S,4S)-5-methyl-2,5-diazabicyclo[2.2.1]heptan-2- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)imino)dimethyl-16-sulfanone and ((6-((4bR,8aR)-2-((3-fluoro- |
| | 4-((1S,4S)-5-methyl-2,5-diazabicyclo[2.2.1]heptan-2-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2- |
| | yl)imino)dimethyl-16-sulfanone |
| 181, 182 | ((6-((4bR,8aR)-2-((3-chloro-4-(1-methylazetidin-3-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2- |
| | yl)imino) dimethyl-16-sulfanone and ((6-((4bS,8aS)-2-((3-chloro-4-(1-methylazetidin-3- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)imino)dimethyl-16-sulfanone |
| 183, 189 | ((6-((4bR,8aR)-2-((4-(azetidin-3-yl)-3-fluorophenyl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone and ((6-((4bS,8aS)-2-((4-(azetidin-3-yl)-3-fluorophenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2- |
| | yl)imino)dimethyl-16-sulfanone |
| 184, 185 | ((6-((4bR,8aR)-2-((3-fluoro-4-(1-methylazetidin-3-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2- |
| | yl)imino)dimethyl-16-sulfanone and ((6-((4bS,8aS)-2-((3-fluoro-4-(1-methylazetidin-3- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)imino)dimethyl-16-sulfanone |
| 186, 194 | dimethyl((6-((4bS,8aS)-4b,7,7-trimethyl-2-((1-(tetrahydro-2H-pyran-4-yl)-1H-pyrazol-4- |
| | yl)amino)-4b,7,8,8a-tetrahydropyrano[3′,4′:4,5]pyrrolo[2,3-d]pyrimidin-9(5H)-yl)pyridin-2- |
| | yl)imino)-16-sulfanone and dimethyl((6-((4bR,8aR)-4b,7,7-trimethyl-2-((1-(tetrahydro-2H- |
| | pyran-4-yl)-1H-pyrazol-4-yl)amino)-4b,7,8,8a-tetrahydropyrano[3′,4′:4,5]pyrrolo[2,3- |
| | d]pyrimidin-9(5H)-yl)pyridin-2-yl)imino)-16-sulfanone |
| 187, 188 | dimethyl((6-((4bS,8aS)-4b,7,7-trimethyl-2-((6-methyl-6,7-dihydro-5H-pyrrolo[3,4-b]pyridin-3- |
| | yl)amino)-4b,7,8,8a-tetrahydropyrano[3′,4′:4,5]pyrrolo[2,3-d]pyrimidin-9(5H)-yl)pyridin-2- |
| | yl)imino)-16-sulfanone and dimethyl((6-((4bR,8aR)-4b,7,7-trimethyl-2-((6-methyl-6,7-dihydro- |
| | 5H-pyrrolo[3,4-b]pyridin-3-yl)amino)-4b,7,8,8a-tetrahydropyrano[3′,4′:4,5]pyrrolo[2,3- |
| | d]pyrimidin-9(5H)-yl)pyridin-2-yl)imino)-16-sulfanone |
| 190, 191 | ((6-((4bR,8aR)-2-((6-(azetidin-3-yl)pyridin-3-yl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone and ((6-((4bS,8aS)-2-((6-(azetidin-3-yl)pyridin-3-yl)amino)-4b,7,7-trimethyl- |
| | 4b,7,8,8a-tetrahydropyrano[3′,4′:4,5]pyrrolo[2,3-d]pyrimidin-9(5H)-yl)pyridin-2- |
| | yl)imino)dimethyl-16-sulfanone |
| 192, 193 | dimethyl((6-((4bR,8aR)-4b,7,7-trimethyl-2-((6-(1-methylazetidin-3-yl)pyridin-3-yl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bS,8aS)-4b,7,7-trimethyl-2-((6-(1-methylazetidin-3-yl)pyridin-3- |
| | yl)amino)-4b,7,8,8a-tetrahydropyrano[3′,4′:4,5]pyrrolo[2,3-d]pyrimidin-9(5H)-yl)pyridin-2- |
| | yl)imino)-16-sulfanone |
| 195, 196 | dimethyl((6-((4bS,8aS)-4b,7,7-trimethyl-2-((1-((R)-1-methylpyrrolidin-3-yl)-1H-pyrazol-4- |
| | yl)amino)-4b,7,8,8a-tetrahydropyrano[3′,4′:4,5]pyrrolo[2,3-d]pyrimidin-9(5H)-yl)pyridin-2- |
| | yl)imino)-16-sulfanone and dimethyl((6-((4bR,8aR)-4b,7,7-trimethyl-2-((1-((R)-1- |
| | methylpyrrolidin-3-yl)-1H-pyrazol-4-yl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 197, 198 | dimethyl((6-((4bS,8aS)-4b,7,7-trimethyl-2-((1-((S)-1-methylpyrrolidin-3-yl)-1H-pyrazol-4- |
| | yl)amino)-4b,7,8,8a-tetrahydropyrano[3′,4′:4,5]pyrrolo[2,3-d]pyrimidin-9(5H)-yl)pyridin-2- |
| | yl)imino)-16-sulfanone and dimethyl((6-((4bR,8aR)-4b,7,7-trimethyl-2-((1-((S)-1- |
| | methylpyrrolidin-3-yl)-1H-pyrazol-4-yl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 199, 200 | dimethyl((6-((4bR,8aR)-4b,7,7-trimethyl-2-((1-((S)-1-methylpiperidin-3-yl)-1H-pyrazol-4- |
| | yl)amino)-4b,7,8,8a-tetrahydropyrano[3′,4′:4,5]pyrrolo[2,3-d]pyrimidin-9(5H)-yl)pyridin-2- |
| | yl)imino)-16-sulfanone and dimethyl((6-((4bS,8aS)-4b,7,7-trimethyl-2-((1-((S)-1- |
| | methylpiperidin-3-yl)-1H-pyrazol-4-yl)amino)-4b,7,8,8a-tetrahydropyrano[3′,4′:4,5]pyrrolo[2,3- |
| | d]pyrimidin-9(5H)-yl)pyridin-2-yl)imino)-16-sulfanone |
| 201, 202 | dimethyl((6-((4bR,8aR)-4b,7,7-trimethyl-2-((1-((R)-1-methylpiperidin-3-yl)-1H-pyrazol-4- |
| | yl)amino)-4b,7,8,8a-tetrahydropyrano[3′,4′:4,5]pyrrolo[2,3-d]pyrimidin-9(5H)-yl)pyridin-2- |
| | yl)imino)-16-sulfanone and dimethyl((6-((4bS,8aS)-4b,7,7-trimethyl-2-((1-((R)-1- |
| | methylpiperidin-3-yl)-1H-pyrazol-4-yl)amino)-4b,7,8,8a-tetrahydropyrano[3′,4′:4,5]pyrrolo[2,3- |
| | d]pyrimidin-9(5H)-yl)pyridin-2-yl)imino)-16-sulfanone |
| 203, 204 | dimethyl((6-((4bS,8aS)-4b,7,7-trimethyl-2-((3-(1-methylpiperidin-4-yl)isoxazol-5-yl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bR,8aR)-4b,7,7-trimethyl-2-((3-(1-methylpiperidin-4-yl)isoxazol- |
| | 5-yl)amino)-4b,7,8,8a-tetrahydropyrano[3′,4′:4,5]pyrrolo[2,3-d]pyrimidin-9(5H)-yl)pyridin-2- |
| | yl)imino)-16-sulfanone |
| 205, 206 | dimethyl((6-((4bR,8aR)-4b,7,7-trimethyl-2-((1-(1-methylpiperidin-4-yl)-1H-imidazol-4- |
| | yl)amino)-4b,7,8,8a-tetrahydropyrano[3′,4′:4,5]pyrrolo[2,3-d]pyrimidin-9(5H)-yl)pyridin-2- |
| | yl)imino)-16-sulfanone and dimethyl((6-((4bS,8aS)-4b,7,7-trimethyl-2-((1-(1-methylpiperidin-4- |
| | yl)-1H-imidazol-4-yl)amino)-4b,7,8,8a-tetrahydropyrano[3′,4′:4,5]pyrrolo[2,3-d]pyrimidin- |
| | 9(5H)-yl)pyridin-2-yl)imino)-16-sulfanone |
| 207, 208 | dimethyl((6-((4bR,8aR)-4b,7,7-trimethyl-2-((1-(1-methylazetidin-3-yl)-1H-pyrazol-4-yl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bS,8aS)-4b,7,7-trimethyl-2-((1-(1-methylazetidin-3-yl)-1H- |
| | pyrazol-4-yl)amino)-4b,7,8,8a-tetrahydropyrano[3′,4′:4,5]pyrrolo[2,3-d]pyrimidin-9(5H)- |
| | yl)pyridin-2-yl)imino)-16-sulfanone |
| 209, 210 | dimethyl((6-((4bS,8aS)-4b,7,7-trimethyl-2-((6-((R)-1-methylpyrrolidin-2-yl)pyridin-3-yl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bR,8aR)-4b,7,7-trimethyl-2-((6-((R)-1-methylpyrrolidin-2- |
| | yl)pyridin-3-yl)amino)-4b,7,8,8a-tetrahydropyrano[3′,4′:4,5]pyrrolo[2,3-d]pyrimidin-9(5H)- |
| | yl)pyridin-2-yl)imino)-16-sulfanone |
| 211, 212 | dimethyl((6-((4bS,8aS)-4b,7,7-trimethyl-2-((6-((R)-pyrrolidin-2-yl)pyridin-3-yl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bR,8aR)-4b,7,7-trimethyl-2-((6-((R)-pyrrolidin-2-yl)pyridin-3- |
| | yl)amino)-4b,7,8,8a-tetrahydropyrano[3′,4′:4,5]pyrrolo[2,3-d]pyrimidin-9(5H)-yl)pyridin-2- |
| | yl)imino)-16-sulfanone |
| 213, 214 | dimethyl((6-((4bS,8aS)-4b,7,7-trimethyl-2-((6-((S)-pyrrolidin-2-yl)pyridin-3-yl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bR,8aR)-4b,7,7-trimethyl-2-((6-((S)-pyrrolidin-2-yl)pyridin-3- |
| | yl)amino)-4b,7,8,8a-tetrahydropyrano[3′,4′:4,5]pyrrolo[2,3-d]pyrimidin-9(5H)-yl)pyridin-2- |
| | yl)imino)-16-sulfanone |
| 215, 216 | dimethyl((6-((4bS,8aS)-4b,7,7-trimethyl-2-((6-((S)-1-methylpyrrolidin-2-yl)pyridin-3-yl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone and dimethyl((6-((4bR,8aR)-4b,7,7-trimethyl-2-((6-((S)-1-methylpyrrolidin-2- |
| | yl)pyridin-3-yl)amino)-4b,7,8,8a-tetrahydropyrano[3′,4′:4,5]pyrrolo[2,3-d]pyrimidin-9(5H)- |
| | yl)pyridin-2-yl)imino)-16-sulfanone |
| 217, 218 | dimethyl((6-((4bR,8aR)-4b,7,7-trimethyl-2-((4-((3aR,6aS)-5-methylhexahydropyrrolo[3,4- |
| | c]pyrrol-2(1H)-yl)phenyl)amino)-4b,7,8,8a-tetrahydropyrano[3′,4′:4,5]pyrrolo[2,3-d]pyrimidin- |
| | 9(5H)-yl)pyridin-2-yl)imino)-16-sulfanone and dimethyl((6-((4bS,8aS)-4b,7,7-trimethyl-2-((4- |
| | ((3aR,6aS)-5-methylhexahydropyrrolo[3,4-c]pyrrol-2(1H)-yl)phenyl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 219, 220 | dimethyl((6-((4bR,8aR)-4b,7,7-trimethyl-2-((4-(6-methyl-2,6-diazaspiro[3.3]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)imino)-16-sulfanone and dimethyl((6-((4bS,8aS)-4b,7,7-trimethyl-2-((4-(6-methyl-2,6- |
| | diazaspiro[3.3]heptan-2-yl)phenyl)amino)-4b,7,8,8a-tetrahydropyrano[3′,4′:4,5]pyrrolo[2,3- |
| | d]pyrimidin-9(5H)-yl)pyridin-2-yl)imino)-16-sulfanone |
| 221 | dimethyl((6-((4bR,8aR)-4b,7,7-trimethyl-2-((2-(1-methylpiperidin-4-yl)thiazol-5-yl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone |
| 223 | dimethyl((6-((4bR,8aR)-4b,7,7-trimethyl-2-((1-(oxetan-3-yl)-1H-pyrazol-4-yl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 225 | dimethyl((6-((4bR,8aR)-4b,7,7-trimethyl-2-((1-((S)-1-methylpyrrolidin-3-yl)-1H-pyrazol-3- |
| | yl)amino)-4b,7,8,8a-tetrahydropyrano[3′,4′:4,5]pyrrolo[2,3-d]pyrimidin-9(5H)-yl)pyridin-2- |
| | yl)imino)-16-sulfanone |
| 227 | dimethyl((6-((4bR,8aR)-4b,7,7-trimethyl-2-((1-((R)-1-methylpyrrolidin-3-yl)-1H-pyrazol-3- |
| | yl)amino)-4b,7,8,8a-tetrahydropyrano[3′,4′:4,5]pyrrolo[2,3-d]pyrimidin-9(5H)-yl)pyridin-2- |
| | yl)imino)-16-sulfanone |
| 229 | dimethyl((6-((4bR,8aR)-4b,7,7-trimethyl-2-((1-(1-methylpiperidin-4-yl)-1H-pyrazol-3- |
| | yl)amino)-4b,7,8,8a-tetrahydropyrano[3′,4′:4,5]pyrrolo[2,3-d]pyrimidin-9(5H)-yl)pyridin-2- |
| | yl)imino)-16-sulfanone |
| 232 | dimethyl((6-((4bR,8aR)-4b,7,7-trimethyl-2-((1-(oxetan-3-yl)-1H-imidazol-4-yl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone |
| 233 | 4-(((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)amino)-1-(tetrahydro-2H- |
| | pyran-4-yl)pyridin-2(1H)-one |
| 234, 235 | (S)-4-methyl-3-(6-((4bS,8aS)-4b,7,7-trimethyl-2-((4-(4-methylpiperazin-1-yl)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and (S)-4-methyl-3-(6-((4bR,8aR)-4b,7,7-trimethyl-2-((4-(4-methylpiperazin-1- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one |
| 236, 237 | (S)-4-methyl-3-(6-((4bS,8aS)-4b,7,7-trimethyl-2-((3-methyl-4-(4-methylpiperazin-1- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one and (S)-4-methyl-3-(6-((4bR,8aR)-4b,7,7-trimethyl-2-((3-methyl-4-(4- |
| | methylpiperazin-1-yl)phenyl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 238, 239 | (S)-4-methyl-3-(6-((4bR,8aR)-4b,7,7-trimethyl-2-((3-methyl-4-(1-methylpiperidin-4- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one and (S)-4-methyl-3-(6-((4bS,8aS)-4b,7,7-trimethyl-2-((3-methyl-4-(1- |
| | methylpiperidin-4-yl)phenyl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 240, 241 | (S)-4-methyl-3-(6-((4bS,8aS)-4b,7,7-trimethyl-2-((6-(4-methylpiperazin-1-yl)pyridin-3- |
| | yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one and (S)-4-methyl-3-(6-((4bR,8aR)-4b,7,7-trimethyl-2-((6-(4- |
| | methylpiperazin-1-yl)pyridin-3-yl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 242, 243 | (S)-4-methyl-3-(6-((4bS,8aS)-4b,7,7-trimethyl-2-((1-(1-methylpiperidin-4-yl)-1H-pyrazol-4- |
| | yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one and (S)-4-methyl-3-(6-((4bR,8aR)-4b,7,7-trimethyl-2-((1-(1- |
| | methylpiperidin-4-yl)-1H-pyrazol-4-yl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 244, 245 | (S)-4-methyl-3-(6-((4bS,8aS)-4b,7,7-trimethyl-2-((4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | (S)-4-methyl-3-(6-((4bR,8aR)-4b,7,7-trimethyl-2-((4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 246, 247 | (S)-4-methyl-3-(6-((4bS,8aS)-4b,7,7-trimethyl-2-((4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | (S)-4-methyl-3-(6-((4bR,8aR)-4b,7,7-trimethyl-2-((4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 248, 249 | (S)-4-methyl-3-(6-((4bS,8aS)-4b,7,7-trimethyl-2-((6-(4-methylpiperazin-1-yl)pyridazin-3- |
| | yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one and (S)-4-methyl-3-(6-((4bR,8aR)-4b,7,7-trimethyl-2-((6-(4- |
| | methylpiperazin-1-yl)pyridazin-3-yl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 250, 251 | (S)-4-methyl-3-(6-((4bS,8aS)-4b,7,7-trimethyl-2-((4-(1-methylpiperidin-4-yl)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and (S)-4-methyl-3-(6-((4bR,8aR)-4b,7,7-trimethyl-2-((4-(1-methylpiperidin-4- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one |
| 252, 253 | (S)-4-methyl-3-(6-((4bS,8aS)-4b,7,7-trimethyl-2-((1-(1-methylpiperidin-4-yl)-1H-1,2,3-triazol- |
| | 4-yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one and (S)-4-methyl-3-(6-((4bR,8aR)-4b,7,7-trimethyl-2-((1-(1- |
| | methylpiperidin-4-yl)-1H-1,2,3-triazol-4-yl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 254, 255 | (S)-4-methyl-3-(6-((4bS,8aS)-4b,7,7-trimethyl-2-((1-(piperidin-4-yl)-1H-pyrazol-4-yl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and (S)-4-methyl-3-(6-((4bR,8aR)-4b,7,7-trimethyl-2-((1-(piperidin-4-yl)-1H-pyrazol-4- |
| | yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one |
| 256, 257 | (S)-4-methyl-3-(6-((4bS,8aS)-4b,7,7-trimethyl-2-((6-((R)-pyrrolidin-3-yl)pyridin-3-yl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and (S)-4-methyl-3-(6-((4bR,8aR)-4b,7,7-trimethyl-2-((6-((R)-pyrrolidin-3-yl)pyridin-3- |
| | yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one |
| 258, 259 | (S)-4-methyl-3-(6-((4bS,8aS)-4b,7,7-trimethyl-2-((6-((S)-pyrrolidin-3-yl)pyridin-3-yl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and (S)-4-methyl-3-(6-((4bR,8aR)-4b,7,7-trimethyl-2-((6-((S)-pyrrolidin-3-yl)pyridin-3- |
| | yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one |
| 260, 261 | (S)-4-methyl-3-(6-((4bR,8aR)-4b,7,7-trimethyl-2-((6-((S)-1-methylpyrrolidin-3-yl)pyridin-3- |
| | yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one and (S)-4-methyl-3-(6-((4bS,8aS)-4b,7,7-trimethyl-2-((6-((S)-1- |
| | methylpyrrolidin-3-yl)pyridin-3-yl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 262, 263 | (S)-4-methyl-3-(6-((4bS,8aS)-4b,7,7-trimethyl-2-((6-((S)-1-methylpyrrolidin-3-yl)pyridin-3- |
| | yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one and (S)-4-methyl-3-(6-((4bR,8aR)-4b,7,7-trimethyl-2-((6-((S)-1- |
| | methylpyrrolidin-3-yl)pyridin-3-yl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 264, 265 | (S)-4-methyl-3-(6-((4bS,8aS)-4b,7,7-trimethyl-2-((6-((R)-1-methylpyrrolidin-3-yl)pyridin-3- |
| | yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one and (S)-4-methyl-3-(6-((4bR,8aR)-4b,7,7-trimethyl-2-((6-((R)-1- |
| | methylpyrrolidin-3-yl)pyridin-3-yl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 266, 267 | (S)-4-methyl-3-(6-((4bS,8aS)-4b,7,7-trimethyl-2-((3-methyl-4-((R)-pyrrolidin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one and (S)-4-methyl-3-(6-((4bR,8aR)-4b,7,7-trimethyl-2-((3-methyl-4-((R)- |
| | pyrrolidin-3-yl)phenyl) amino)-4b,7,8,8a-tetrahydropyrano[3′,4′:4,5]pyrrolo[2,3-d]pyrimidin- |
| | 9(5H)-yl)pyridin-2-yl)oxazolidin-2-one |
| | yl)phenyl)amino)-4b,7,8,8a-tetrahydropyrano[3',4':4,5]pyrrolo[2,3-d]pyrimidin-9(5H)-yl)pyridin- |
| 268, 269 | (S)-4-methyl-3-(6-((4bS,8aS)-4b,7,7-trimethyl-2-((3-methyl-4-((S)-pyrrolidin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one and (S)-4-methyl-3-(6-((4bR,8aR)-4b,7,7-trimethyl-2-((3-methyl-4-((S)- |
| | pyrrolidin-3-yl)phenyl)amino)-4b,7,8,8a-tetrahydropyrano[3′,4′:4,5]pyrrolo[2,3-d]pyrimidin- |
| | 9(5H)-yl)pyridin-2-yl)oxazolidin-2-one |
| 270, 271 | (S)-4-methyl-3-(6-((4bS,8aS)-4b,7,7-trimethyl-2-((4-(4-methyl-2-oxopiperazin-1- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one and (S)-4-methyl-3-(6-((4bR,8aR)-4b,7,7-trimethyl-2-((4-(4-methyl-2- |
| | oxopiperazin-1-yl)phenyl)amino)-4b,7,8,8a-tetrahydropyrano[3′,4′:4,5]pyrrolo[2,3-d]pyrimidin- |
| | 9(5H)-yl)pyridin-2-yl)oxazolidin-2-one |
| 272, 273 | (S)-4-methyl-3-(6-((4bS,8aS)-4b,7,7-trimethyl-2-((1-(1-methylpiperidin-4-yl)-2-oxo-1,2- |
| | dihydropyridin-4-yl)amino)-4b,7,8,8a-tetrahydropyrano[3′,4′:4,5]pyrrolo[2,3-d]pyrimidin-9(5H)- |
| | yl)pyridin-2-yl)oxazolidin-2-one and (S)-4-methyl-3-(6-((4bR,8aR)-4b,7,7-trimethyl-2-((1-(1- |
| | methylpiperidin-4-yl)-2-oxo-1,2-dihydropyridin-4-yl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 274, 275 | (S)-4-methyl-3-(6-((4bS,8aS)-4b,7,7-trimethyl-2-((4-((S)-pyrrolidin-3-yl)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and (S)-4-methyl-3-(6-((4bR,8aR)-4b,7,7-trimethyl-2-((4-((S)-pyrrolidin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one |
| 276, 277 | (S)-3-(6-((4bS,8aS)-2-((4-((1R,4R)-2,5-diazabicyclo[2.2.1]heptan-2-yl)phenyl)amino)-4b,7,7- |
| | trimethyl-4b,7,8,8a-tetrahydropyrano[3′,4′:4,5]pyrrolo[2,3-d]pyrimidin-9(5H)-yl)pyridin-2-yl)-4- |
| | methyloxazolidin-2-one and (S)-3-(6-((4bS,8aS)-2-((4-((1S,4S)-2,5- |
| | diazabicyclo[2.2.1]heptan-2-yl)phenyl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)-4-methyloxazolidin-2- |
| | one |
| 278, 279 | (S)-4-methyl-3-(6-((4bS,8aS)-4b,7,7-trimethyl-2-((4-((R)-pyrrolidin-3-yl)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and (S)-4-methyl-3-(6-((4bR,8aR)-4b,7,7-trimethyl-2-((4-((R)-pyrrolidin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one |
| 280, 281 | (S)-4-methyl-3-(6-((4bS,8aS)-4b,7,7-trimethyl-2-((6-(((S)-1-methylpyrrolidin-3-yl)oxy)pyridin- |
| | 3-yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one and (S)-4-methyl-3-(6-((4bR,8aR)-4b,7,7-trimethyl-2-((6-(((S)-1- |
| | methylpyrrolidin-3-yl)oxy)pyridin-3-yl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 282, 283 | (S)-4-methyl-3-(6-((4bS,8aS)-4b,7,7-trimethyl-2-((6-(((R)-1-methylpyrrolidin-3-yl)oxy)pyridin- |
| | 3-yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one and (S)-4-methyl-3-(6-((4bR,8aR)-4b,7,7-trimethyl-2-((6-(((R)-1- |
| | methylpyrrolidin-3-yl)oxy)pyridin-3-yl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 284, 285 | (S)-4-methyl-3-(6-((4bS,8aS)-4b,7,7-trimethyl-2-((6-(((S)-pyrrolidin-3-yl)oxy)pyridin-3- |
| | yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one and (S)-4-methyl-3-(6-((4bR,8aR)-4b,7,7-trimethyl-2-((6-(((S)-pyrrolidin-3- |
| | yl)oxy)pyridin-3-yl)amino)-4b,7,8,8a-tetrahydropyrano[3′,4′:4,5]pyrrolo[2,3-d]pyrimidin-9(5H)- |
| | yl)pyridin-2-yl)oxazolidin-2-one |
| 286, 287 | (S)-4-methyl-3-(6-((4bS,8aS)-4b,7,7-trimethyl-2-((6-(((R)-pyrrolidin-3-yl)oxy)pyridin-3- |
| | yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one and (S)-4-methyl-3-(6-((4bR,8aR)-4b,7,7-trimethyl-2-((6-(((R)-pyrrolidin-3- |
| | yl)oxy)pyridin-3-yl)amino)-4b,7,8,8a-tetrahydropyrano[3′,4′:4,5]pyrrolo[2,3-d]pyrimidin-9(5H)- |
| | yl)pyridin-2-yl)oxazolidin-2-one |
| 288, 289 | (S)-4-methyl-3-(6-((4bS,8aS)-4b,7,7-trimethyl-2-((3-methyl-4-((1R,4R)-5-methyl-2,5- |
| | diazabicyclo[2.2.1]heptan-2-yl)phenyl)amino)-46,7,8,8a- |
| | tetrahydropyrano[3′,4′:4,5]pyrrolo[2,3-d]pyrimidin-9(5H)-yl)pyridin-2-yl)oxazolidin-2-one and |
| | (S)-4-methyl-3-(6-((4bR,8aR)-4b,7,7-trimethyl-2-((3-methyl-4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 290, 291 | (S)-3-(6-((4bS,8aS)-2-((3-fluoro-4-((1R,4R)-5-methyl-2,5-diazabicyclo[2.2.1]heptan-2- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)-4-methyloxazolidin-2-one and (S)-3-(6-((4bR,8aR)-2-((3- |
| | fluoro-4-((1R,4R)-5-methyl-2,5-diazabicyclo[2.2.1]heptan-2-yl)phenyl)amino)-46,7,7- |
| | trimethyl-4b,7,8,8a-tetrahydropyrano[3′,4′:4,5]pyrrolo[2,3-d]pyrimidin-9(5H)-yl)pyridin-2-yl)-4- |
| | methyloxazolidin-2-one |
| 292, 293 | (S)-3-(6-((4bS,8aS)-2-((3-fluoro-4-((1S,4S)-5-methyl-2,5-diazabicyclo[2.2.1]heptan-2- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)-4-methyloxazolidin-2-one and (S)-3-(6-((4bR,8aR)-2-((3- |
| | fluoro-4-((1S,4S)-5-methyl-2,5-diazabicyclo[2.2.1]heptan-2-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2-yl)-4- |
| | methyloxazolidin-2-one |
| 294, 295 | (S)-4-methyl-3-(6-((4bS,8aS)-4b,7,7-trimethyl-2-((3-methyl-4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | (S)-4-methyl-3-(6-((4bR,8aR)-4b,7,7-trimethyl-2-((3-methyl-4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 296, 297 | (S)-4-methyl-3-(6-((4bS,8aS)-4b,7,7-trimethyl-2-((6-(1-methylpiperidin-4-yl)pyridin-3- |
| | yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one and (S)-4-methyl-3-(6-((4bR,8aR)-4b,7,7-trimethyl-2-((6-(1- |
| | methylpiperidin-4-yl)pyridin-3-yl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 298, 299 | (S)-4-methyl-3-(6-((4bS,8aS)-4b,7,7-trimethyl-2-((4-(4-methyl-1,4-diazepan-1- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one and (S)-4-methyl-3-(6-((4bR,8aR)-4b,7,7-trimethyl-2-((4-(4-methyl-1,4- |
| | diazepan-1-yl)phenyl)amino)-4b,7,8,8a-tetrahydropyrano[3′,4′:4,5]pyrrolo[2,3-d]pyrimidin- |
| | 9(5H)-yl)pyridin-2-yl)oxazolidin-2-one |
| 300, 301 | (S)-3-(6-((4bS,8aS)-2-((6-(tert-butyl)pyridin-3-yl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)-4-methyloxazolidin-2- |
| | one and (S)-3-(6-((4bR,8aR)-2-((6-(tert-butyl)pyridin-3-yl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)-4-methyloxazolidin-2- |
| | ne |
| 302, 303 | (S)-4-methyl-3-(6-((4bS,8aS)-4b,7,7-trimethyl-2-((4-(3-methyloxetan-3-yl)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and (S)-4-methyl-3-(6-((4bR,8aR)-4b,7,7-trimethyl-2-((4-(3-methyloxetan-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one |
| 304, 305 | (S)-4-methyl-3-(6-((4bS,8aS)-4b,7,7-trimethyl-2-((4-((S)-1-methylpyrrolidin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one and (S)-4-methyl-3-(6-((4bR,8aR)-4b,7,7-trimethyl-2-((4-((S)-1- |
| | methylpyrrolidin-3-yl)phenyl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 306, 307 | (S)-4-methyl-3-(6-((4bR,8aR)-4b,7,7-trimethyl-2-((4-((R)-1-methylpyrrolidin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one and (S)-4-methyl-3-(6-((4bS,8aS)-4b,7,7-trimethyl-2-((4-((R)-1- |
| | methylpyrrolidin-3-yl)phenyl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 308, 310 | (S)-3-(6-((4bS,8aS)-2-((4-(azetidin-3-yl)phenyl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)-4-methyloxazolidin-2- |
| | one and (S)-3-(6-((4bR,8aR)-2-((4-(azetidin-3-yl)phenyl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)-4-methyloxazolidin-2- |
| | one |
| 309, 311 | (S)-4-methyl-3-(6-((4bS,8aS)-4b,7,7-trimethyl-2-((4-(1-methylazetidin-3-yl)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and (S)-4-methyl-3-(6-((4bR,8aR)-4b,7,7-trimethyl-2-((4-(1-methylazetidin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one |
| 312, 313 | (S)-3-(6-((4bS,8aS)-2-((4-((R)-3,4-dimethylpiperazin-1-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2-yl)-4- |
| | methyloxazolidin-2-one and (S)-3-(6-((4bR,8aR)-2-((4-((R)-3,4-dimethylpiperazin-1- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)-4-methyloxazolidin-2-one |
| 314, 315 | (S)-3-(6-((4bS,8aS)-2-((4-((S)-3,4-dimethylpiperazin-1-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2-yl)-4- |
| | methyloxazolidin-2-one and (S)-3-(6-((4bR,8aR)-2-((4-((S)-3,4-dimethylpiperazin-1- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)-4-methyloxazolidin-2-one |
| 316, 317 | (S)-4-methyl-3-(6-((4bS,8aS)-4b,7,7-trimethyl-2-((4-((S)-3-(methylamino)pyrrolidin-1- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one and (S)-4-methyl-3-(6-((4bR,8aR)-4b,7,7-trimethyl-2-((4-((S)-3- |
| | (methylamino)pyrrolidin-1-yl)phenyl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 318, 319 | (S)-4-methyl-3-(6-((4bS,8aS)-4b,7,7-trimethyl-2-((4-((R)-3-(methylamino)pyrrolidin-1- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one and (S)-4-methyl-3-(6-((4bR,8aR)-4b,7,7-trimethyl-2-((4-((R)-3- |
| | (methylamino)pyrrolidin-1-yl)phenyl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 320, 321 | (S)-2-(methylamino)-3-(4-(((4bS,8aS)-4b,7,7-trimethyl-9-(6-((S)-4-methyl-2-oxooxazolidin-3- |
| | yl)pyridin-2-yl)-4b,5,7,8,8a,9-hexahydropyrano[3′,4′:4,5]pyrrolo[2,3-d]pyrimidin-2- |
| | yl)amino)phenyl)propanoic acid and (S)-2-(methylamino)-3-(4-(((4bR,8aR)-4b,7,7-trimethyl-9- |
| | (6-((S)-4-methyl-2-oxooxazolidin-3-yl)pyridin-2-yl)-4b,5,7,8,8a,9- |
| | hexahydropyrano[3′,4′:4,5]pyrrolo[2,3-d]pyrimidin-2-yl)amino)phenyl)propanoic acid |
| 322, 323 | (R)-2-(methylamino)-3-(4-(((4bS,8aS)-4b,7,7-trimethyl-9-(6-((S)-4-methyl-2-oxooxazolidin-3- |
| | yl)pyridin-2-yl)-4b,5,7,8,8a,9-hexahydropyrano[3′,4′:4,5]pyrrolo[2,3-d]pyrimidin-2- |
| | yl)amino)phenyl)propanoic acid and (R)-2-(methylamino)-3-(4-(((4bR,8aR)-4b,7,7-trimethyl- |
| | 9-(6-((S)-4-methyl-2-oxooxazolidin-3-yl)pyridin-2-yl)-4b,5,7,8,8a,9- |
| | hexahydropyrano[3′,4′:4,5]pyrrolo[2,3-d]pyrimidin-2-yl)amino)phenyl)propanoic acid |
| 324, 325 | (S)-4-methyl-3-(6-((4bS,8aS)-4b,7,7-trimethyl-2-((6-(piperidin-4-yloxy)pyridin-3-yl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and (S)-4-methyl-3-(6-((4bR,8aR)-4b,7,7-trimethyl-2-((6-(piperidin-4-yloxy)pyridin-3- |
| | yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one |
| 326, 327 | (S)-4-methyl-3-(6-((4bS,8aS)-4b,7,7-trimethyl-2-((4-(((R)-pyrrolidin-3-yl)oxy)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and (S)-4-methyl-3-(6-((4bR,8aR)-4b,7,7-trimethyl-2-((4-(((R)-pyrrolidin-3- |
| | yl)oxy)phenyl)amino)-4b,7,8,8a-tetrahydropyrano[3′,4′:4,5]pyrrolo[2,3-d]pyrimidin-9(5H)- |
| | yl)pyridin-2-yl)oxazolidin-2-one |
| 328, 329 | (S)-4-methyl-3-(6-((4bS,8aS)-4b,7,7-trimethyl-2-((4-(((S)-pyrrolidin-3-yl)oxy)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and (S)-4-methyl-3-(6-((4bR,8aR)-4b,7,7-trimethyl-2-((4-(((S)-pyrrolidin-3- |
| | yl)oxy)phenyl)amino)-4b,7,8,8a-tetrahydropyrano[3′,4′:4,5]pyrrolo[2,3-d]pyrimidin-9(5H)- |
| | yl)pyridin-2-yl)oxazolidin-2-one |
| 330, 331 | (S)-4-methyl-3-(6-((4bS,8aS)-4b,7,7-trimethyl-2-((4-(((R)-1-methylpyrrolidin-3- |
| | yl)oxy)phenyl)amino)-4b,7,8,8a-tetrahydropyrano[3′,4′:4,5]pyrrolo[2,3-d]pyrimidin-9(5H)- |
| | yl)pyridin-2-yl)oxazolidin-2-one and (S)-4-methyl-3-(6-((4bR,8aR)-4b,7,7-trimethyl-2-((4-(((R)- |
| | 1-methylpyrrolidin-3-yl)oxy)phenyl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 332, 333 | (S)-4-methyl-3-(6-((4bS,8aS)-4b,7,7-trimethyl-2-((4-(((S)-1-methylpyrrolidin-3- |
| | yl)oxy)phenyl)amino)-4b,7,8,8a-tetrahydropyrano[3′,4′:4,5]pyrrolo[2,3-d]pyrimidin-9(5H)- |
| | yl)pyridin-2-yl)oxazolidin-2-one and (S)-4-methyl-3-(6-((4bR,8aR)-4b,7,7-trimethyl-2-((4-(((S)- |
| | 1-methylpyrrolidin-3-yl)oxy)phenyl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 334, 335 | (S)-4-methyl-3-(6-((4bS,8aS)-4b,7,7-trimethyl-2-((6-((1-methylpiperidin-4-yl)oxy)pyridin-3- |
| | yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one and (S)-4-methyl-3-(6-((4bR,8aR)-4b,7,7-trimethyl-2-((6-((1- |
| | methylpiperidin-4-yl)oxy)pyridin-3-yl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 336, 337 | (S)-4-methyl-3-(6-((4bS,8aS)-4b,7,7-trimethyl-2-((3-methyl-4-(((R)-pyrrolidin-3- |
| | yl)oxy)phenyl)amino)-4b,7,8,8a-tetrahydropyrano[3′,4′:4,5]pyrrolo[2,3-d]pyrimidin-9(5H)- |
| | yl)pyridin-2-yl)oxazolidin-2-one and (S)-4-methyl-3-(6-((4bR,8aR)-4b,7,7-trimethyl-2-((3- |
| | methyl-4-(((R)-pyrrolidin-3-yl)oxy)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 338, 339 | (S)-4-methyl-3-(6-((4bS,8aS)-4b,7,7-trimethyl-2-((3-methyl-4-(((S)-pyrrolidin-3- |
| | yl)oxy)phenyl)amino)-4b,7,8,8a-tetrahydropyrano[3′,4′:4,5]pyrrolo[2,3-d]pyrimidin-9(5H)- |
| | yl)pyridin-2-yl)oxazolidin-2-one and (S)-4-methyl-3-(6-((4bR,8aR)-4b,7,7-trimethyl-2-((3- |
| | methyl-4-(((S)-pyrrolidin-3-yl)oxy)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 340, 341 | (S)-4-methyl-3-(6-((4bS,8aS)-4b,7,7-trimethyl-2-((1-methyl-2-oxo-1,2-dihydropyridin-4- |
| | yl)amino)-4b,7,8,8a-tetrahydropyrano[3′,4′:4,5]pyrrolo[2,3-d]pyrimidin-9(5H)-yl)pyridin-2- |
| | yl)oxazolidin-2-one and (S)-4-methyl-3-(6-((4bR,8aR)-4b,7,7-trimethyl-2-((1-methyl-2-oxo- |
| | 1,2-dihydropyridin-4-yl)amino)-4b,7,8,8a-tetrahydropyrano[3′,4′:4,5]pyrrolo[2,3-d]pyrimidin- |
| | 9(5H)-yl)pyridin-2-yl)oxazolidin-2-one |
| 342, 343 | (S)-3-(6-((4bS,8aS)-2-((6-(azetidin-3-yloxy)pyridin-3-yl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)-4-methyloxazolidin-2- |
| | one and (S)-3-(6-((4bR,8aR)-2-((6-(azetidin-3-yloxy)pyridin-3-yl)amino)-4b,7,7-trimethyl- |
| | 4b,7,8,8a-tetrahydropyrano[3′,4′:4,5]pyrrolo[2,3-d]pyrimidin-9(5H)-yl)pyridin-2-yl)-4- |
| | methyloxazolidin-2-one |
| 344, 345 | (S)-4-methyl-3-(6-((4bS,8aS)-4b,7,7-trimethyl-2-((3-methyl-4-(((S)-1-methylpyrrolidin-3- |
| | yl)oxy)phenyl)amino)-4b,7,8,8a-tetrahydropyrano[3′,4′:4,5]pyrrolo[2,3-d]pyrimidin-9(5H)- |
| | yl)pyridin-2-yl)oxazolidin-2-one and (S)-4-methyl-3-(6-((4bR,8aR)-4b,7,7-trimethyl-2-((3- |
| | methyl-4-(((S)-1-methylpyrrolidin-3-yl)oxy)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 346, 347 | (S)-4-methyl-3-(6-((4bS,8aS)-4b,7,7-trimethyl-2-((3-methyl-4-(((R)-1-methylpyrrolidin-3- |
| | yl)oxy)phenyl)amino)-4b,7,8,8a-tetrahydropyrano[3′,4′:4,5]pyrrolo[2,3-d]pyrimidin-9(5H)- |
| | yl)pyridin-2-yl)oxazolidin-2-one and (S)-4-methyl-3-(6-((4bR,8aR)-4b,7,7-trimethyl-2-((3- |
| | methyl-4-(((R)-1-methylpyrrolidin-3-yl)oxy)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 348, 349 | (S)-3-(6-((4bS,8aS)-2-((4-((R)-1,3-dimethylpyrrolidin-3-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2-yl)-4- |
| | methyloxazolidin-2-one and (S)-3-(6-((4bR,8aR)-2-((4-((R)-1,3-dimethylpyrrolidin-3- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)-4-methyloxazolidin-2-one |
| 350, 351 | (S)-3-(6-((4bS,8aS)-2-((4-((S)-1,3-dimethylpyrrolidin-3-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2-yl)-4- |
| | methyloxazolidin-2-one and (S)-3-(6-((4bR,8aR)-2-((4-((S)-1,3-dimethylpyrrolidin-3- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)-4-methyloxazolidin-2-one |
| 352, 353 | (S)-4-methyl-3-(6-((4bR,8aR)-4b,7,7-trimethyl-2-((4-((1R,5S)-6-methyl-3,6- |
| | diazabicyclo[3.1.1]heptan-3-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | (S)-4-methyl-3-(6-((4bS,8aS)-4b,7,7-trimethyl-2-((4-((1R,5S)-6-methyl-3,6- |
| | diazabicyclo[3.1.1]heptan-3-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 354, 355 | (S)-4-methyl-3-(6-((4bS,8aS)-4b,7,7-trimethyl-2-((4-(6-methyl-2,6-diazaspiro[3.3]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one and (S)-4-methyl-3-(6-((4bR,8aR)-4b,7,7-trimethyl-2-((4-(6-methyl-2,6- |
| | diazaspiro[3.3]heptan-2-yl)phenyl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 356, 357 | (S)-3-(6-((4bR,8aR)-2-amino-4b,7,7-trimethyl-4b,7,8,8a- |
| | tetrahydropyrano[3′,4′:4,5]pyrrolo[2,3-d]pyrimidin-9(5H)-yl)pyridin-2-yl)-4-methyloxazolidin-2- |
| | one and (S)-3-(6-((4bS,8aS)-2-amino-4b,7,7-trimethyl-4b,7,8,8a- |
| | tetrahydropyrano[3′,4′:4,5]pyrrolo[2,3-d]pyrimidin-9(5H)-yl)pyridin-2-yl)-4-methyloxazolidin-2- |
| | one |
| 359, 360 | (S)-4-methyl-3-(6-((4bS,8aS)-4b,7,7-trimethyl-2-((4-((methylamino)methyl)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and (S)-4-methyl-3-(6-((4bR,8aR)-4b,7,7-trimethyl-2-((4- |
| | ((methylamino)methyl)phenyl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 36 | ((6-((4bR,8aR)-2-(imidazo[1,2-b]pyridazin-6-ylamino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 362 | ((6-((4bR,8aR)-2-([1,2,3]triazolo[1,5-a]pyridin-6-ylamino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 363 | 4-(((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)amino)-1-methylpyridin- |
| | 2(1H)-one |
| 364 | dimethyl((6-((4bR,8aR)-4b,7,7-trimethyl-2-((6-methylpyridazin-3-yl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 365 | 6-(((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)amino)-2- |
| | methylpyridazin-3(2H)-one |
| 366 | dimethyl((6-((4bR,8aR)-4b,7,7-trimethyl-2-(pyrazin-2-ylamino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 367 | dimethyl((6-((4bR,8aR)-4b,7,7-trimethyl-2-((4-methylpyridin-2-yl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 368 | ((6-((4bR,8aR)-2-((3-fluoropyridin-2-yl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 369 | ((6-((4bR,8aR)-2-((5-fluoropyridin-3-yl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 370 | dimethyl((6-((4bR,8aR)-4b,7,7-trimethyl-2-((6-methylpyridazin-4-yl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 371 | dimethyl((6-((4bR,8aR)-4b,7,7-trimethyl-2-((5-methylpyrazin-2-yl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 372 | ((6-((4bR,8aR)-2-((6-methoxypyridazin-3-yl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 373 | dimethyl((6-((4bR,8aR)-4b,7,7-trimethyl-2-((2-methylpyrimidin-5-yl)amino)-4b,7,8,8a- |
| | tetrahydropyrano [3′,4′:4,5]pyrrolo[2,3-d]pyrimidin-9(5H)-yl)pyridin-2-yl)imino)-16-sulfanone |
| 374 | 1-(difluoromethyl)-4-(((4bR,8aR)-9-(6-((dimethyl(oxo)-16-sulfaneylidene)amino)pyridin-2-yl)- |
| | 4b,7,7-trimethyl-4b,5,7,8,8a,9-hexahydropyrano[3′,4′:4,5]pyrrolo[2,3-d]pyrimidin-2- |
| | yl)amino)pyridin-2(1H)-one |
| 375 | 4-(((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)amino)-1-((R)- |
| | tetrahydrofuran-3-yl)pyridin-2(1H)-one |
| 376 | 4-(((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)amino)-1-((S)- |
| | tetrahydrofuran-3-yl)pyridin-2(1H)-one |
| 377 | ((6-((4bR,8aR)-2-((6-(difluoromethyl)pyridazin-3-yl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 378 | 4-(((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)amino)-1-(methyl- |
| | d3)pyridin-2(1H)-one |
| 379 | ((6-((4bR,8aR)-2-(imidazo[1,2-a]pyridin-7-ylamino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 380 | 1-cyclopropyl-4-(((4bR,8aR)-9-(6-((dimethyl(oxo)-16-sulfaneylidene)amino)pyridin-2-yl)- |
| | 4b,7,7-trimethyl-4b,5,7,8,8a,9-hexahydropyrano[3′,4′:4,5]pyrrolo[2,3-d]pyrimidin-2- |
| | yl)amino)pyridin-2(1H)-one |
| 381 | ((6-((4bR,8aR)-2-(benzo[d]isoxazol-6-ylamino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 382 | ((6-((4bR,8aR)-2-(imidazo[1,2-a]pyrazin-6-ylamino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 383 | dimethyl((6-((4bR,8aR)-4b,7,7-trimethyl-2-((5-methyl-5H-pyrrolo[2,3-b]pyrazin-2-yl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone |
| 384 | ((6-((4bR,8aR)-2-([1,2,4]triazolo[1,5-a]pyridin-6-ylamino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 385 | ((6-((4bR,8aR)-2-([1,2,4]triazolo[1,5-a]pyridin-7-ylamino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 386 | 1-(tetrahydro-2H-pyran-4-yl)-4-(((4bR,8aR)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-yl)amino)pyridin-2(1H)-one |
| 387 | 4-(((4bR,8aR)-9-(6-((dimethylamino)methyl)pyridin-2-yl)-4b,7,7-trimethyl-4b,5,7,8,8a,9- |
| | hexahydropyrano[3′,4′:4,5]pyrrolo[2,3-d]pyrimidin-2-yl)amino)-1-(tetrahydro-2H-pyran-4- |
| | yl)pyridin-2(1H)-one |
| 388, 389 | 1-((R)-tetrahydrofuran-3-yl)-4-(((4bR,8aR)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-yl)amino)pyridin-2(1H)-one and 1-((S)-tetrahydrofuran-3-yl)-4-(((4bR,8aR)- |
| | 4b,7,7-trimethyl-9-(6-((methylamino)methyl)pyridin-2-yl)-4b,5,7,8,8a,9- |
| | hexahydropyrano[3′,4′:4,5]pyrrolo[2,3-d]pyrimidin-2-yl)amino)pyridin-2(1H)-one |
| 390, 391 | 1-(tetrahydro-2H-pyran-4-yl)-4-(((4bR,8aR)-4b,7,7-trimethyl-9-(6-((S)-1- |
| | (methylamino)ethyl)pyridin-2-yl)-4b,5,7,8,8a,9-hexahydropyrano[3′,4′:4,5]pyrrolo[2,3- |
| | d]pyrimidin-2-yl)amino)pyridin-2(1H)-one and 1-(tetrahydro-2H-pyran-4-yl)-4-(((4bR,8aR)- |
| | 4b,7,7-trimethyl-9-(6-((R)-1-(methylamino)ethyl)pyridin-2-yl)-4b,5,7,8,8a,9- |
| | hexahydropyrano[3′,4′:4,5]pyrrolo[2,3-d]pyrimidin-2-yl)amino)pyridin-2(1H)-one |
| 392, 394 | 1-(tetrahydro-2H-pyran-4-yl)-4-(((4bR,8aR)-4b,7,7-trimethyl-9-(6-((S)-pyrrolidin-2-yl)pyridin-2- |
| | yl)-4b,5,7,8,8a,9-hexahydropyrano[3′,4′:4,5]pyrrolo[2,3-d]pyrimidin-2-yl)amino)pyridin-2(1H)- |
| | one and 1-(tetrahydro-2H-pyran-4-yl)-4-(((4bR,8aR)-4b,7,7-trimethyl-9-(6-((R)-pyrrolidin-2- |
| | yl)pyridin-2-yl)-4b,5,7,8,8a,9-hexahydropyrano[3′,4′:4,5]pyrrolo[2,3-d]pyrimidin-2- |
| | yl)amino)pyridin-2(1H)-one |
| 393, 395 | 1-(tetrahydro-2H-pyran-4-yl)-4-(((4bR,8aR)-4b,7,7-trimethyl-9-(6-((S)-1-methylpyrrolidin-2- |
| | yl)pyridin-2-yl)-4b,5,7,8,8a,9-hexahydropyrano[3′,4′:4,5]pyrrolo[2,3-d]pyrimidin-2- |
| | yl)amino)pyridin-2(1H)-one and 1-(tetrahydro-2H-pyran-4-yl)-4-(((4bR,8aR)-4b,7,7-trimethyl- |
| | 9-(6-((R)-1-methylpyrrolidin-2-yl)pyridin-2-yl)-4b,5,7,8,8a,9- |
| | hexahydropyrano[3′,4′:4,5]pyrrolo[2,3-d]pyrimidin-2-yl)amino)pyridin-2(1H)-one |
| 396 | (1R,2R)-N-((4bR,8aR)-9-(6-((dimethyl(oxo)-16-sulfaneylidene)amino)pyridin-2-yl)-4b,7,7- |
| | trimethyl-4b,5,7,8,8a,9-hexahydropyrano[3′,4′:4,5]pyrrolo[2,3-d]pyrimidin-2-yl)-2- |
| | phenylcyclopropane-1-carboxamide |
| 397 | (1S,2S)-N-((4bR,8aR)-9-(6-((dimethyl(oxo)-16-sulfaneylidene)amino)pyridin-2-yl)-4b,7,7- |
| | trimethyl-4b,5,7,8,8a,9-hexahydropyrano[3′,4′:4,5]pyrrolo[2,3-d]pyrimidin-2-yl)-2- |
| | phenylcyclopropane-1-carboxamide |
| 398 | N-((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)acetamide |
| 399 | N-((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2- |
| | yl)cyclopropanecarboxamide |
| 400 | N-((4bR,8aR)-4b,7,7-trimethyl-9-(6-((1-oxidotetrahydro-116-thiophen-1-ylidene)amino)pyridin- |
| | 2-yl)-4b,5,7,8,8a,9-hexahydropyrano[3′,4′:4,5]pyrrolo[2,3-d]pyrimidin-2- |
| | yl)cyclopropanecarboxamide |
| 401 | (1R,2R)-N-((4bR,8aR)-9-(6-((dimethyl(oxo)-16-sulfaneylidene)amino)pyridin-2-yl)-4b,7,7- |
| | trimethyl-4b,5,7,8,8a,9-hexahydropyrano[3′,4′:4,5]pyrrolo[2,3-d]pyrimidin-2-yl)-2-(pyridin-3- |
| | yl)cyclopropane-1-carboxamide |
| 402 | (1S,2S)-N-((4bR,8aR)-9-(6-((dimethyl(oxo)-16-sulfaneylidene)amino)pyridin-2-yl)-4b,7,7- |
| | trimethyl-4b,5,7,8,8a,9-hexahydropyrano[3′,4′:4,5]pyrrolo[2,3-d]pyrimidin-2-yl)-2-(pyridin-3- |
| | yl)cyclopropane-1-carboxamide |
| 403 | (1R,2R)-N-((4bR,8aR)-9-(6-((dimethyl(oxo)-16-sulfaneylidene)amino)pyridin-2-yl)-4b,7,7- |
| | trimethyl-4b,5,7,8,8a,9-hexahydropyrano[3′,4′:4,5]pyrrolo[2,3-d]pyrimidin-2-yl)-2-(1-methyl- |
| | 1H-pyrazol-4-yl)cyclopropane-1-carboxamide |
| 404 | N-((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)benzamide |
| 405 | N-((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)nicotinamide |
| 406 | (1S,2S)-N-((4bR,8aR)-9-(6-((dimethyl(oxo)-16-sulfaneylidene)amino)pyridin-2-yl)-4b,7,7- |
| | trimethyl-4b,5,7,8,8a,9-hexahydropyrano[3′,4′:4,5]pyrrolo[2,3-d]pyrimidin-2-yl)-2-(1-methyl- |
| | 1H-pyrazol-4-yl)cyclopropane-1-carboxamide |
| 407 | N-((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)picolinamide |
| 408 | N-((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)-1-methyl-1H-pyrazole-4- |
| | carboxamide |
| 409 | N-((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)-2-fluorobenzamide |
| 410 | N-((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)-1-fluorocyclopropane-1- |
| | carboxamide |
| 411 | (1S,2R)-N-((4bR,8aR)-9-(6-((dimethyl(oxo)-16-sulfaneylidene)amino)pyridin-2-yl)-4b,7,7- |
| | trimethyl-4b,5,7,8,8a,9-hexahydropyrano[3′,4′:4,5]pyrrolo[2,3-d]pyrimidin-2-yl)-2-(tetrahydro- |
| | 2H-pyran-4-yl)cyclopropane-1-carboxamide |
| 412 | (1R,2S)-N-((4bR,8aR)-9-(6-((dimethyl(oxo)-16-sulfaneylidene)amino)pyridin-2-yl)-4b,7,7- |
| | trimethyl-4b,5,7,8,8a,9-hexahydropyrano[3′,4′:4,5]pyrrolo[2,3-d]pyrimidin-2-yl)-2-(tetrahydro- |
| | 2H-pyran-4-yl)cyclopropane-1-carboxamide |
| 413, 414 | 2-(6-((4bR,8aR)-4b,7,7-trimethyl-2-((4-((1R,4R)-5-methyl-2,5-diazabicyclo[2.2.1]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)propan-2-ol and 2-(6-((4bS,8aS)-4b,7,7-trimethyl-2-((4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)propan-2-ol |
| 415, 416 | 2-(6-((4bS,8aS)-4b,7,7-trimethyl-2-((4-((1S,4S)-5-methyl-2,5-diazabicyclo[2.2.1]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)propan-2-ol and 2-(6-((4bR,8aR)-4b,7,7-trimethyl-2-((4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)propan-2-ol |
| 417, 418 | 2-(6-((4bR,8aR)-4b,7,7-trimethyl-2-((4-(((R)-pyrrolidin-3-yl)oxy)phenyl)amino)-4b,7,8,8a- |
| | tetrahydropyrano[3′,4′:4,5]pyrrolo[2,3-d]pyrimidin-9(5H)-yl)pyridin-2-yl)propan-2-ol and 2-(6- |
| | ((4bS,8aS)-4b,7,7-trimethyl-2-((4-(((R)-pyrrolidin-3-yl)oxy)phenyl)amino)-4b,7,8,8a- |
| | tetrahydropyrano[3′,4′:4,5]pyrrolo[2,3-d]pyrimidin-9(5H)-yl)pyridin-2-yl)propan-2-ol |
| 419, 420 | 2-(6-((4bR,8aR)-4b,7,7-trimethyl-2-((4-(((S)-pyrrolidin-3-yl)oxy)phenyl)amino)-4b,7,8,8a- |
| | tetrahydropyrano[3′,4′:4,5]pyrrolo[2,3-d]pyrimidin-9(5H)-yl)pyridin-2-yl)propan-2-ol and 2-(6- |
| | ((4bS,8aS)-4b,7,7-trimethyl-2-((4-(((S)-pyrrolidin-3-yl)oxy)phenyl)amino)-4b,7,8,8a- |
| | tetrahydropyrano[3′,4′:4,5]pyrrolo[2,3-d]pyrimidin-9(5H)-yl)pyridin-2-yl)propan-2-ol |
| 421, 422 | 2-(6-((4bR,8aR)-4b,7,7-trimethyl-2-((4-(((S)-1-methylpyrrolidin-3-yl)oxy)phenyl)amino)- |
| | 4b,7,8,8a-tetrahydropyrano[3′,4′:4,5]pyrrolo[2,3-d]pyrimidin-9(5H)-yl)pyridin-2-yl)propan-2-ol |
| | and 2-(6-((4bS,8aS)-4b,7,7-trimethyl-2-((4-(((S)-1-methylpyrrolidin-3-yl)oxy)phenyl)amino)- |
| | 4b,7,8,8a-tetrahydropyrano[3′,4′:4,5]pyrrolo[2,3-d]pyrimidin-9(5H)-yl)pyridin-2-yl)propan-2-ol |
| 423, 424 | 2-(6-((4bR,8aR)-4b,7,7-trimethyl-2-((4-(((R)-1-methylpyrrolidin-3-yl)oxy)phenyl)amino)- |
| | 4b,7,8,8a-tetrahydropyrano[3′,4′:4,5]pyrrolo[2,3-d]pyrimidin-9(5H)-yl)pyridin-2-yl)propan-2-ol |
| | and 2-(6-((4bS,8aS)-4b,7,7-trimethyl-2-((4-(((R)-1-methylpyrrolidin-3-yl)oxy)phenyl)amino)- |
| | 4b,7,8,8a-tetrahydropyrano[3′,4′:4,5]pyrrolo[2,3-d]pyrimidin-9(5H)-yl)pyridin-2-yl)propan-2-ol |
| 425, 426 | 2-(6-((4bR,8aR)-4b,7,7-trimethyl-2-((3-methyl-4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)propan-2-ol and 2-(6- |
| | ((4bS,8aS)-4b,7,7-trimethyl-2-((3-methyl-4-((1R,4R)-5-methyl-2,5-diazabicyclo[2.2.1]heptan- |
| | 2-yl)phenyl)amino)-4b,7,8,8a-tetrahydropyrano[3′,4′:4,5]pyrrolo[2,3-d]pyrimidin-9(5H)- |
| | yl)pyridin-2-yl)propan-2-ol |
| 427, 428 | 2-(6-((4bR,8aR)-2-((3-fluoro-4-((1R,4R)-5-methyl-2,5-diazabicyclo[2.2.1]heptan-2- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)propan-2-ol and 2-(6-((4bS,8aS)-2-((3-fluoro-4-((1R,4R)-5- |
| | methyl-2,5-diazabicyclo[2.2.1]heptan-2-yl)phenyl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)propan-2-ol |
| 429, 430 | 2-(6-((4bR,8aR)-4b,7,7-trimethyl-2-((3-methyl-4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)propan-2-ol and 2-(6- |
| | ((4bS,8aS)-4b,7,7-trimethyl-2-((3-methyl-4-((1S,4S)-5-methyl-2,5-diazabicyclo[2.2.1]heptan- |
| | 2-yl)phenyl)amino)-4b,7,8,8a-tetrahydropyrano[3′,4′:4,5]pyrrolo[2,3-d]pyrimidin-9(5H)- |
| | yl)pyridin-2-yl)propan-2-ol |
| 431, 432 | 2-(6-((4bR,8aR)-2-((3-fluoro-4-((1S,4S)-5-methyl-2,5-diazabicyclo[2.2.1]heptan-2- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)propan-2-ol and 2-(6-((4bS,8aS)-2-((3-fluoro-4-((1S,4S)-5- |
| | methyl-2,5-diazabicyclo[2.2.1]heptan-2-yl)phenyl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)propan-2-ol |
| 433, 434 | 2-(6-((4bR,8aR)-2-((3-chloro-4-((1R,4R)-5-methyl-2,5-diazabicyclo[2.2.1]heptan-2- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)propan-2-ol and 2-(6-((4bS,8aS)-2-((3-chloro-4-((1R,4R)-5- |
| | methyl-2,5-diazabicyclo[2.2.1]heptan-2-yl)phenyl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)propan-2-ol |
| 435, 436 | 2-(6-((4bR,8aR)-2-((3-chloro-4-((1S,4S)-5-methyl-2,5-diazabicyclo[2.2.1]heptan-2- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)propan-2-ol and 2-(6-((4bS,8aS)-2-((3-chloro-4-((1S,4S)-5- |
| | methyl-2,5-diazabicyclo[2.2.1]heptan-2-yl)phenyl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)propan-2-ol |
| 437, 438 | 2-(6-((4bR,8aR)-4b,7,7-trimethyl-2-((4-((3aR,6aR)-1-methylhexahydropyrrolo[3,4-b]pyrrol- |
| | 5(1H)-yl)phenyl)amino)-4b,7,8,8a-tetrahydropyrano[3′,4′:4,5]pyrrolo[2,3-d]pyrimidin-9(5H)- |
| | yl)pyridin-2-yl)propan-2-ol and 2-(6-((4bS,8aS)-4b,7,7-trimethyl-2-((4-((3aR,6aR)-1- |
| | methylhexahydropyrrolo[3,4-b]pyrrol-5(1H)-yl)phenyl)amino)-4b,7,8,8a- |
| | tetrahydropyrano[3′,4′:4,5]pyrrolo[2,3-d]pyrimidin-9(5H)-yl)pyridin-2-yl)propan-2-ol |
| 439, 440 | 2-(6-((4bR,8aR)-4b,7,7-trimethyl-2-((4-((3aS,6aS)-1-methylhexahydropyrrolo[3,4-b]pyrrol- |
| | 5(1H)-yl)phenyl)amino)-4b,7,8,8a-tetrahydropyrano[3′,4′:4,5]pyrrolo[2,3-d]pyrimidin-9(5H)- |
| | yl)pyridin-2-yl)propan-2-ol and 2-(6-((4bS,8aS)-4b,7,7-trimethyl-2-((4-((3aS,6aS)-1- |
| | methylhexahydropyrrolo[3,4-b]pyrrol-5(1H)-yl)phenyl)amino)-4b,7,8,8a- |
| | tetrahydropyrano[3′,4′:4,5]pyrrolo[2,3-d]pyrimidin-9(5H)-yl)pyridin-2-yl)propan-2-ol |
| 441, 442 | 2-(6-((4bR,8aR)-4b,7,7-trimethyl-2-((4-((1R,5S)-6-methyl-3,6-diazabicyclo[3.1.1]heptan-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)propan-2-ol and 2-(6-((4bS,8aS)-4b,7,7-trimethyl-2-((4-((1R,5S)-6-methyl-3,6- |
| | diazabicyclo[3.1.1]heptan-3-yl)phenyl)amino)-4b,7,8,8a- |
| | tetrahydropyrano[3′,4′:4,5]pyrrolo[2,3-d]pyrimidin-9(5H)-yl)pyridin-2-yl)propan-2-ol |
| 443, 444 | 2-(6-((4bR,8aR)-4b,7,7-trimethyl-2-((4-(6-methyl-2,6-diazaspiro[3.3]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)propan-2-ol and 2-(6-((4bS,8aS)-4b,7,7-trimethyl-2-((4-(6-methyl-2,6- |
| | diazaspiro[3.3]heptan-2-yl)phenyl)amino)-4b,7,8,8a-tetrahydropyrano[3′,4′:4,5]pyrrolo[2,3- |
| | d]pyrimidin-9(5H)-yl)pyridin-2-yl)propan-2-ol |
| 445, 446 | 2-(6-((4bS,8aS)-4b,7,7-trimethyl-2-((4-((3aR,6aS)-5-methylhexahydropyrrolo[3,4-c]pyrrol- |
| | 2(1H)-yl)phenyl)amino)-4b,7,8,8a-tetrahydropyrano[3′,4′:4,5]pyrrolo[2,3-d]pyrimidin-9(5H)- |
| | yl)pyridin-2-yl)propan-2-ol and 2-(6-((4bR,8aR)-4b,7,7-trimethyl-2-((4-((3aR,6aS)-5- |
| | methylhexahydropyrrolo[3,4-c]pyrrol-2(1H)-yl)phenyl)amino)-4b,7,8,8a- |
| | tetrahydropyrano[3′,4′:4,5]pyrrolo[2,3-d]pyrimidin-9(5H)-yl)pyridin-2-yl)propan-2-ol |
| 447, 448 | (4bS,8aS)-4b,7,7-trimethyl-N-(3-methyl-4-(4-methylpiperazin-1-yl)phenyl)-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bR,8aR)-4b,7,7-trimethyl-N-(3-methyl-4-(4-methylpiperazin-1- |
| | yl)phenyl)-9-(6-((methylamino)methyl)pyridin-2-yl)-4b,5,7,8,8a,9- |
| | hexahydropyrano[3′,4′:4,5]pyrrolo[2,3-d]pyrimidin-2-amine |
| 449, 450 | 1-methyl-4-(4-(((4bS,8aS)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)phenyl)piperazin- |
| | 2-one and 1-methyl-4-(4-(((4bR,8aR)-4b,7,7-trimethyl-9-(6-((methylamino)methyl)pyridin-2- |
| | yl)-4b,5,7,8,8a,9-hexahydropyrano[3′,4′:4,5]pyrrolo[2,3-d]pyrimidin-2- |
| | yl)amino)phenyl)piperazin-2-one |
| 451, 452 | (4bS,8aS)-4b,7,7-trimethyl-9-(6-((methylamino)methyl)pyridin-2-yl)-N-(4-((R)-pyrrolidin-3- |
| | yl)phenyl)-4b,5,7,8,8a,9-hexahydropyrano[3′,4′:4,5]pyrrolo[2,3-d]pyrimidin-2-amine and |
| | (4bR,8aR)-4b,7,7-trimethyl-9-(6-((methylamino)methyl)pyridin-2-yl)-N-(4-((R)-pyrrolidin-3- |
| | yl)phenyl)-4b,5,7,8,8a,9-hexahydropyrano[3′,4′:4,5]pyrrolo[2,3-d]pyrimidin-2-amine |
| 453, 454 | (4bS,8aS)-4b,7,7-trimethyl-9-(6-((methylamino)methyl)pyridin-2-yl)-N-(4-((S)-pyrrolidin-3- |
| | yl)phenyl)-4b,5,7,8,8a,9-hexahydropyrano[3′,4′:4,5]pyrrolo[2,3-d]pyrimidin-2-amine and |
| | (4bR,8aR)-4b,7,7-trimethyl-9-(6-((methylamino)methyl)pyridin-2-yl)-N-(4-((S)-pyrrolidin-3- |
| | yl)phenyl)-4b,5,7,8,8a,9-hexahydropyrano[3′,4′:4,5]pyrrolo[2,3-d]pyrimidin-2-amine |
| 455, 456 | 1-methyl-4-(((4bS,8aS)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)pyridin-2(1H)-one |
| | and 1-methyl-4-(((4bR,8aR)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)pyridin-2(1H)-one |
| 457, 458 | 2-methyl-2-(4-(((4bS,8aS)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)phenyl)propanoic |
| | acid and 2-methyl-2-(4-(((4bR,8aR)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)phenyl)propanoic |
| | acid |
| 459, 460 | (4bS,8aS)-4b,7,7-trimethyl-N-(6-(1-methyl-1H-pyrazol-4-yl)pyridin-3-yl)-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bR,8aR)-4b,7,7-trimethyl-N-(6-(1-methyl-1H-pyrazol-4-yl)pyridin- |
| | 3-yl)-9-(6-((methylamino)methyl)pyridin-2-yl)-4b,5,7,8,8a,9- |
| | hexahydropyrano[3′,4′:4,5]pyrrolo[2,3-d]pyrimidin-2-amine |
| 461, 462 | 1-methyl-5-(((4bS,8aS)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)pyridin-2(1H)-one |
| | and 1-methyl-5-(((4bR,8aR)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)amino)pyridin-2(1H)-one |
| 463, 464 | (4bS,8aS)-4b,7,7-trimethyl-N-(3-methyl-4-(1-methyl-1H-pyrazol-4-yl)phenyl)-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bR,8aR)-4b,7,7-trimethyl-N-(3-methyl-4-(1-methyl-1H-pyrazol-4- |
| | yl)phenyl)-9-(6-((methylamino)methyl)pyridin-2-yl)-4b,5,7,8,8a,9- |
| | hexahydropyrano[3′,4′:4,5]pyrrolo[2,3-d]pyrimidin-2-amine |
| 465, 466 | (4bS,8aS)-N-(3-fluoro-4-(1H-imidazol-1-yl)phenyl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bR,8aR)-N-(3-fluoro-4-(1H-imidazol-1-yl)phenyl)-4b,7,7-trimethyl- |
| | 9-(6-((methylamino)methyl)pyridin-2-yl)-4b,5,7,8,8a,9-hexahydropyrano[3′,4′:4,5]pyrrolo[2,3- |
| | d]pyrimidin-2-amine |
| 467, 468 | (4bS,8aS)-N-(6-(1H-imidazol-1-yl)pyridin-3-yl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bR,8aR)-N-(6-(1H-imidazol-1-yl)pyridin-3-yl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine |
| 469, 470 | (4bS,8aS)-4b,7,7-trimethyl-9-(6-((methylamino)methyl)pyridin-2-yl)-N-(3-morpholinophenyl)- |
| | 4b,5,7,8,8a,9-hexahydropyrano[3′,4′:4,5]pyrrolo[2,3-d]pyrimidin-2-amine and (4bR,8aR)- |
| | 4b,7,7-trimethyl-9-(6-((methylamino)methyl)pyridin-2-yl)-N-(3-morpholinophenyl)- |
| | 4b,5,7,8,8a,9-hexahydropyrano[3′,4′:4,5]pyrrolo[2,3-d]pyrimidin-2-amine |
| 471, 472 | (4bS,8aS)-4b,7,7-trimethyl-N-(1-methyl-1H-pyrazol-4-yl)-9-(6-((methylamino)methyl)pyridin- |
| | 2-yl)-4b,5,7,8,8a,9-hexahydropyrano[3′,4′:4,5]pyrrolo[2,3-d]pyrimidin-2-amine and (4bR,8aR)- |
| | 4b,7,7-trimethyl-N-(1-methyl-1H-pyrazol-4-yl)-9-(6-((methylamino)methyl)pyridin-2-yl)- |
| | 4b,5,7,8,8a,9-hexahydropyrano[3′,4′:4,5]pyrrolo[2,3-d]pyrimidin-2-amine |
| 473, 474 | (4bR,8aR)-4b,7,7-trimethyl-9-(6-((methylamino)methyl)pyridin-2-yl)-N-(4-((R)-3- |
| | methylmorpholino)phenyl)-4b,5,7,8,8a,9-hexahydropyrano[3′,4′:4,5]pyrrolo[2,3-d]pyrimidin-2- |
| | amine and (4bS,8aS)-4b,7,7-trimethyl-9-(6-((methylamino)methyl)pyridin-2-yl)-N-(4-((R)-3- |
| | methylmorpholino)phenyl)-4b,5,7,8,8a,9-hexahydropyrano[3′,4′:4,5]pyrrolo[2,3-d]pyrimidin-2- |
| | amine |
| 475, 476 | (4bS,8aS)-4b,7,7-trimethyl-9-(6-((methylamino)methyl)pyridin-2-yl)-N-(4-((S)-3- |
| | methylmorpholino)phenyl)-4b,5,7,8,8a,9-hexahydropyrano[3′,4′:4,5]pyrrolo[2,3-d]pyrimidin-2- |
| | amine and (4bR,8aR)-4b,7,7-trimethyl-9-(6-((methylamino)methyl)pyridin-2-yl)-N-(4-((S)-3- |
| | methylmorpholino)phenyl)-4b,5,7,8,8a,9-hexahydropyrano[3′,4′:4,5]pyrrolo[2,3-d]pyrimidin-2- |
| | amine |
| 477, 478 | (4bS,8aS)-4b,7,7-trimethyl-9-(6-((methylamino)methyl)pyridin-2-yl)-N-(6-methylpyridazin-4- |
| | yl)-4b,5,7,8,8a,9-hexahydropyrano[3′,4′:4,5]pyrrolo[2,3-d]pyrimidin-2-amine and (4bR,8aR)- |
| | 4b,7,7-trimethyl-9-(6-((methylamino)methyl)pyridin-2-yl)-N-(6-methylpyridazin-4-yl)- |
| | 4b,5,7,8,8a,9-hexahydropyrano[3′,4′:4,5]pyrrolo[2,3-d]pyrimidin-2-amine |
| 479, 480 | (4bR,8aR)-N-(4-((1R,5S)-3-oxa-6-azabicyclo[3.1.1]heptan-6-yl)phenyl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bS,8aS)-N-(4-((1R,5S)-3-oxa-6-azabicyclo[3.1.1]heptan-6- |
| | yl)phenyl)-4b,7,7-trimethyl-9-(6-((methylamino)methyl)pyridin-2-yl)-4b,5,7,8,8a,9- |
| | hexahydropyrano[3′,4′:4,5]pyrrolo[2,3-d]pyrimidin-2-amine |
| 481, 482 | (4bR,8aR)-N-(4-((1R,5S)-6-oxa-3-azabicyclo[3.1.1]heptan-3-yl)phenyl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine and (4bS,8aS)-N-(4-((1R,5S)-6-oxa-3-azabicyclo[3.1.1]heptan-3- |
| | yl)phenyl)-4b,7,7-trimethyl-9-(6-((methylamino)methyl)pyridin-2-yl)-4b,5,7,8,8a,9- |
| | hexahydropyrano[3′,4′:4,5]pyrrolo[2,3-d]pyrimidin-2-amine |
| 483 | dimethyl((6-((4bR,8aR)-4b,7,7-trimethyl-2-((5-(4-methylpiperazin-1-yl)pyridin-3-yl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone |
| 484 | dimethyl((6-((4bR,8aR)-4b,7,7-trimethyl-2-((1-methyl-1H-pyrazol-4-yl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 485 | dimethyl((6-((4bR,8aR)-4b,7,7-trimethyl-2-((5-(1-methylpiperidin-4-yl)pyridin-3-yl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone |
| 486 | dimethyl((6-((4bR,8aR)-4b,7,7-trimethyl-2-((3-(4-methylpiperazin-1-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone |
| 487 | ((6-((4bR,8aR)-2-((1-(difluoromethyl)-1H-pyrazol-4-yl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 488 | dimethyl((6-((4bR,8aR)-4b,7,7-trimethyl-2-((1-(trifluoromethyl)-1H-pyrazol-4-yl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone |
| 490 | ((6-((4bR,8aR)-2-((5-fluoro-1-methyl-1H-pyrazol-4-yl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 491 | dimethyl((6-((4bR,8aR)-4b,7,7-trimethyl-2-((6-methylpyridin-3-yl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 492 | dimethyl((6-((4bR,8aR)-4b,7,7-trimethyl-2-((5-methylpyridin-3-yl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 493 | dimethyl((6-((4bR,8aR)-4b,7,7-trimethyl-2-((6-morpholinopyridin-3-yl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 494 | ((6-((4bR,8aR)-2-((4-(2,6-diazaspiro[3.3]heptan-2-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2- |
| | yl)imino)dimethyl-16-sulfanone |
| 495 | 5-(((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)amino)-1-methylpyridin- |
| | 2(1H)-one |
| 496 | dimethyl((6-((4bR,8aR)-4b,7,7-trimethyl-2-((4-morpholinophenyl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 497 | ((6-((4bR,8aR)-2-((4-((1S,4S)-2-oxa-5-azabicyclo[2.2.1]heptan-5-yl)phenyl)amino)-4b,7,7- |
| | trimethyl-4b,7,8,8a-tetrahydropyrano[3′,4′:4,5]pyrrolo[2,3-d]pyrimidin-9(5H)-yl)pyridin-2- |
| | yl)imino)dimethyl-16-sulfanone |
| 498 | ((6-((4bR,8aR)-2-((4-((1R,4R)-2-oxa-5-azabicyclo[2.2.1]heptan-5-yl)phenyl)amino)-4b,7,7- |
| | trimethyl-4b,7,8,8a-tetrahydropyrano[3′,4′:4,5]pyrrolo[2,3-d]pyrimidin-9(5H)-yl)pyridin-2- |
| | yl)imino)dimethyl-16-sulfanone |
| 499 | ((6-((4bR,8aR)-2-((4-((R)-3,4-dimethylpiperazin-1-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2- |
| | yl)imino)dimethyl-16-sulfanone |
| 500 | ((6-((4bR,8aR)-2-((4-((S)-3,4-dimethylpiperazin-1-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2- |
| | yl)imino)dimethyl-16-sulfanone |
| 501 | dimethyl((6-((4bR,8aR)-4b,7,7-trimethyl-2-((4-(4-methyl-1,4-diazepan-1-yl)phenyl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone |
| 502 | dimethyl((6-((4bR,8aR)-4b,7,7-trimethyl-2-((4-((3aR,6aR)-1-methylhexahydropyrrolo[3,4- |
| | b]pyrrol-5(1H)-yl)phenyl)amino)-4b,7,8,8a-tetrahydropyrano[3′,4′:4,5]pyrrolo[2,3-d]pyrimidin- |
| | 9(5H)-yl)pyridin-2-yl)imino)-16-sulfanone |
| 503 | dimethyl((6-((4bR,8aR)-4b,7,7-trimethyl-2-((4-((3aS,6aS)-1-methylhexahydropyrrolo[3,4- |
| | b]pyrrol-5(1H)-yl)phenyl)amino)-4b,7,8,8a-tetrahydropyrano[3′,4′:4,5]pyrrolo[2,3-d]pyrimidin- |
| | 9(5H)-yl)pyridin-2-yl)imino)-16-sulfanone |
| 504 | ((6-((4bR,8aR)-2-(imidazo[1,2-a]pyridin-6-ylamino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 505 | ((6-((4bR,8aR)-2-((4-((R)-hexahydropyrrolo[1,2-a]pyrazin-2(1H)-yl)phenyl)amino)-4b,7,7- |
| | trimethyl-4b,7,8,8a-tetrahydropyrano[3′,4′:4,5]pyrrolo[2,3-d]pyrimidin-9(5H)-yl)pyridin-2- |
| | yl)imino)dimethyl-16-sulfanone |
| 506 | dimethyl((6-((4bR,8aR)-4b,7,7-trimethyl-2-((4-((1R,5S)-6-methyl-3,6- |
| | diazabicyclo[3.1.1]heptan-3-yl)phenyl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 507 | ((6-((4bR,8aR)-2-((4-((S)-hexahydropyrrolo[1,2-a]pyrazin-2(1H)-yl)phenyl)amino)-4b,7,7- |
| | trimethyl-4b,7,8,8a-tetrahydropyrano[3′,4′:4,5]pyrrolo[2,3-d]pyrimidin-9(5H)-yl)pyridin-2- |
| | yl)imino)dimethyl-16-sulfanone |
| 508 | ((6-((4bR,8aR)-2-((4-(2-oxa-6-azaspiro[3.3]heptan-6-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2- |
| | yl)imino)dimethyl-16-sulfanone |
| 509 | dimethyl((6-((4bR,8aR)-4b,7,7-trimethyl-2-((6-(4-methylpiperazin-1-yl)pyridin-3-yl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone |
| 510 | dimethyl((6-((4bR,8aR)-4b,7,7-trimethyl-2-((5-(4-methylpiperazin-1-yl)pyridin-2-yl)amino)- |
| | 4b,7,8,8a-tetrahydropyrano[3′,4′:4,5]pyrrolo[2,3-d]pyrimidin-9(5H)-yl)pyridin-2-yl)imino)-16- |
| | sulfanone |
| 511 | ((6-((4bR,8aR)-2-((4-(1,4-diazabicyclo[3.2.1]octan-4-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2- |
| | yl)imino)dimethyl-16-sulfanone |
| 512 | dimethyl((6-((4bR,8aR)-4b,7,7-trimethyl-2-(pyridin-4-ylamino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 513 | dimethyl((6-((4bR,8aR)-4b,7,7-trimethyl-2-((1-methyl-1H-pyrazol-3-yl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 514 | dimethyl((6-((4bR,8aR)-4b,7,7-trimethyl-2-((1-methyl-1H-1,2,3-triazol-4-yl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 515 | dimethyl((6-((4bR,8aR)-4b,7,7-trimethyl-2-((2-methyl-2H-1,2,3-triazol-4-yl)amino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 516 | ((6-((4bR,8aR)-2-((1-ethyl-1H-1,2,3-triazol-4-yl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 517 | 3-(6-((4bR,8aR)-4b,7,7-trimethyl-2-((4-((1R,4R)-5-methyl-2,5-diazabicyclo[2.2.1]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxetan-3-ol |
| 518 | 3-(6-((4bR,8aR)-4b,7,7-trimethyl-2-((4-((1S,4S)-5-methyl-2,5-diazabicyclo[2.2.1]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxetan-3-ol |
| 519 | (4bR,8aR)-4b,7,7-trimethyl-9-(1-methyl-1H-pyrazol-3-yl)-N-(4-((1S,4S)-5-methyl-2,5- |
| | diazabicyclo[2.2.1]heptan-2-yl)phenyl)-4b,5,7,8,8a,9-hexahydropyrano[3′,4′:4,5]pyrrolo[2,3- |
| | d]pyrimidin-2-amine |
| 520 | (4bR,8aR)-4b,7,7-trimethyl-9-(1-methyl-1H-pyrazol-3-yl)-N-(4-((1R,4R)-5-methyl-2,5- |
| | diazabicyclo[2.2.1]heptan-2-yl)phenyl)-4b,5,7,8,8a,9-hexahydropyrano[3′,4′:4,5]pyrrolo[2,3- |
| | d]pyrimidin-2-amine |
| 521, 522 | (4bS,8aS)-4b,7,7-trimethyl-N-(3-methyl-4-morpholinophenyl)-9-(6-((R)-pyrrolidin-2-yl)pyridin- |
| | 2-yl)-4b,5,7,8,8a,9-hexahydropyrano[3′,4′:4,5]pyrrolo[2,3-d]pyrimidin-2-amine and (4bR,8aR)- |
| | 4b,7,7-trimethyl-N-(3-methyl-4-morpholinophenyl)-9-(6-((R)-pyrrolidin-2-yl)pyridin-2-yl)- |
| | 4b,5,7,8,8a,9-hexahydropyrano[3′,4′:4,5]pyrrolo[2,3-d]pyrimidin-2-amine |
| 523, 524 | (4bR,8aR)-9-(6-((dimethylamino)methyl)pyridin-2-yl)-4b,7,7-trimethyl-N-(4- |
| | morpholinophenyl)-4b,5,7,8,8a,9-hexahydropyrano[3′,4′:4,5]pyrrolo[2,3-d]pyrimidin-2-amine |
| | and (4bS,8aS)-9-(6-((dimethylamino)methyl)pyridin-2-yl)-4b,7,7-trimethyl-N-(4- |
| | morpholinophenyl)-4b,5,7,8,8a,9-hexahydropyrano[3′,4′:4,5]pyrrolo[2,3-d]pyrimidin-2-amine |
| 525, 526 | (4bS,8aS)-4b,7,7-trimethyl-N-(4-morpholinophenyl)-9-(6-((S)-pyrrolidin-2-yl)pyridin-2-yl)- |
| | 4b,5,7,8,8a,9-hexahydropyrano[3′,4′:4,5]pyrrolo[2,3-d]pyrimidin-2-amine and (4bR,8aR)- |
| | 4b,7,7-trimethyl-N-(4-morpholinophenyl)-9-(6-((S)-pyrrolidin-2-yl)pyridin-2-yl)-4b,5,7,8,8a,9- |
| | hexahydropyrano[3′,4′:4,5]pyrrolo[2,3-d]pyrimidin-2-amine |
| 527, 528 | (4bS,8aS)-4b,7,7-trimethyl-N-(4-morpholinophenyl)-9-(6-((R)-pyrrolidin-2-yl)pyridin-2-yl)- |
| | 4b,5,7,8,8a,9-hexahydropyrano[3′,4′:4,5]pyrrolo[2,3-d]pyrimidin-2-amine and (4bR,8aR)- |
| | 4b,7,7-trimethyl-N-(4-morpholinophenyl)-9-(6-((R)-pyrrolidin-2-yl)pyridin-2-yl)-4b,5,7,8,8a,9- |
| | hexahydropyrano[3′,4′:4,5]pyrrolo[2,3-d]pyrimidin-2-amine |
| 529, 530 | (4bR,8aR)-4b,7,7-trimethyl-N-(3-methyl-4-morpholinophenyl)-9-(6-((S)-pyrrolidin-2-yl)pyridin- |
| | 2-yl)-4b,5,7,8,8a,9-hexahydropyrano[3′,4′:4,5]pyrrolo[2,3-d]pyrimidin-2-amine and (4bS,8aS)- |
| | 4b,7,7-trimethyl-N-(3-methyl-4-morpholinophenyl)-9-(6-((S)-pyrrolidin-2-yl)pyridin-2-yl)- |
| | 4b,5,7,8,8a,9-hexahydropyrano[3′,4′:4,5]pyrrolo[2,3-d]pyrimidin-2-amine |
| 531, 532 | (4bR,8aR)-4b,7,7-trimethyl-N-(4-morpholinophenyl)-9-(6-(piperidin-4-yl)pyridin-2-yl)- |
| | 4b,5,7,8,8a,9-hexahydropyrano[3′,4′:4,5]pyrrolo[2,3-d]pyrimidin-2-amine and (4bS,8aS)- |
| | 4b,7,7-trimethyl-N-(4-morpholinophenyl)-9-(6-(piperidin-4-yl)pyridin-2-yl)-4b,5,7,8,8a,9- |
| | hexahydropyrano[3′,4′:4,5]pyrrolo[2,3-d]pyrimidin-2-amine |
| 533, 534 | (4bR,8aR)-4b,7,7-trimethyl-9-(6-(1-methylpiperidin-4-yl)pyridin-2-yl)-N-(4-morpholinophenyl)- |
| | 4b,5,7,8,8a,9-hexahydropyrano[3′,4′:4,5]pyrrolo[2,3-d]pyrimidin-2-amine and (4bS,8aS)- |
| | 4b,7,7-trimethyl-9-(6-(1-methylpiperidin-4-yl)pyridin-2-yl)-N-(4-morpholinophenyl)- |
| | 4b,5,7,8,8a,9-hexahydropyrano[3′,4′:4,5]pyrrolo[2,3-d]pyrimidin-2-amine |
| 535, 536 | (4bR,8aR)-4b,7,7-trimethyl-9-(6-(4-methylpiperazin-1-yl)pyridin-2-yl)-N-(4-morpholinophenyl)- |
| | 4b,5,7,8,8a,9-hexahydropyrano[3′,4′:4,5]pyrrolo[2,3-d]pyrimidin-2-amine and (4bS,8aS)- |
| | 4b,7,7-trimethyl-9-(6-(4-methylpiperazin-1-yl)pyridin-2-yl)-N-(4-morpholinophenyl)- |
| | 4b,5,7,8,8a,9-hexahydropyrano[3′,4′:4,5]pyrrolo[2,3-d]pyrimidin-2-amine |
| 537, 538 | (4bR,8aR)-4b,7,7-trimethyl-9-(6-((S)-1-(methylamino)ethyl)pyridin-2-yl)-N-(4- |
| | morpholinophenyl)-4b,5,7,8,8a,9-hexahydropyrano[3′,4′:4,5]pyrrolo[2,3-d]pyrimidin-2-amine |
| | and (4bS,8aS)-4b,7,7-trimethyl-9-(6-((S)-1-(methylamino)ethyl)pyridin-2-yl)-N-(4- |
| | morpholinophenyl)-4b,5,7,8,8a,9-hexahydropyrano[3′,4′:4,5]pyrrolo[2,3-d]pyrimidin-2-amine |
| 539, 540 | (4bR,8aR)-4b,7,7-trimethyl-N-(4-morpholinophenyl)-9-(6-((R)-pyrrolidin-3-yl)pyridin-2-yl)- |
| | 4b,5,7,8,8a,9-hexahydropyrano[3′,4′:4,5]pyrrolo[2,3-d]pyrimidin-2-amine and (4bS,8aS)- |
| | 4b,7,7-trimethyl-N-(4-morpholinophenyl)-9-(6-((R)-pyrrolidin-3-yl)pyridin-2-yl)-4b,5,7,8,8a,9- |
| | hexahydropyrano[3′,4′:4,5]pyrrolo[2,3-d]pyrimidin-2-amine |
| 541, 542 | (4bS,8aS)-4b,7,7-trimethyl-N-(4-morpholinophenyl)-9-(6-((S)-pyrrolidin-3-yl)pyridin-2-yl)- |
| | 4b,5,7,8,8a,9-hexahydropyrano[3′,4′:4,5]pyrrolo[2,3-d]pyrimidin-2-amine and (4bR,8aR)- |
| | 4b,7,7-trimethyl-N-(4-morpholinophenyl)-9-(6-((S)-pyrrolidin-3-yl)pyridin-2-yl)-4b,5,7,8,8a,9- |
| | hexahydropyrano[3′,4′:4,5]pyrrolo[2,3-d]pyrimidin-2-amine |
| 543, 544 | (4bR,8aR)-4b,7,7-trimethyl-9-(6-(1-(methylamino)cyclopropyl)pyridin-2-yl)-N-(4- |
| | morpholinophenyl)-4b,5,7,8,8a,9-hexahydropyrano[3′,4′:4,5]pyrrolo[2,3-d]pyrimidin-2-amine |
| | and (4bS,8aS)-4b,7,7-trimethyl-9-(6-(1-(methylamino)cyclopropyl)pyridin-2-yl)-N-(4- |
| | morpholinophenyl)-4b,5,7,8,8a,9-hexahydropyrano[3′,4′:4,5]pyrrolo[2,3-d]pyrimidin-2-amine |
| 545, 546 | (4bR,8aR)-9-(6-(3-fluoroazetidin-3-yl)pyridin-2-yl)-4b,7,7-trimethyl-N-(4-morpholinophenyl)- |
| | 4b,5,7,8,8a,9-hexahydropyrano[3′,4′:4,5]pyrrolo[2,3-d]pyrimidin-2-amine and (4bS,8aS)-9-(6- |
| | (3-fluoroazetidin-3-yl)pyridin-2-yl)-4b,7,7-trimethyl-N-(4-morpholinophenyl)-4b,5,7,8,8a,9- |
| | hexahydropyrano[3′,4′:4,5]pyrrolo[2,3-d]pyrimidin-2-amine |
| 547, 548 | (4bR,8aR)-4b,7,7-trimethyl-9-(4-((methylamino)methyl)pyridin-2-yl)-N-(4-morpholinophenyl)- |
| | 4b,5,7,8,8a,9-hexahydropyrano[3′,4′:4,5]pyrrolo[2,3-d]pyrimidin-2-amine and (4bS,8aS)- |
| | 4b,7,7-trimethyl-9-(4-((methylamino)methyl)pyridin-2-yl)-N-(4-morpholinophenyl)- |
| | 4b,5,7,8,8a,9-hexahydropyrano[3′,4′:4,5]pyrrolo[2,3-d]pyrimidin-2-amine |
| 549, 550 | (4bR,8aR)-4b,7,7-trimethyl-9-(6-((R)-1-(methylamino)ethyl)pyridin-2-yl)-N-(4- |
| | morpholinophenyl)-4b,5,7,8,8a,9-hexahydropyrano[3′,4′:4,5]pyrrolo[2,3-d]pyrimidin-2-amine |
| | and (4bS,8aS)-4b,7,7-trimethyl-9-(6-((R)-1-(methylamino)ethyl)pyridin-2-yl)-N-(4- |
| | morpholinophenyl)-4b,5,7,8,8a,9-hexahydropyrano[3′,4′:4,5]pyrrolo[2,3-d]pyrimidin-2-amine |
| 551, 552 | (4bR,8aR)-4b,7,7-trimethyl-9-((S)-7-(methylamino)-6,7-dihydro-5H-cyclopenta[b]pyridin-2-yl)- |
| | N-(4-morpholinophenyl)-4b,5,7,8,8a,9-hexahydropyrano[3′,4′:4,5]pyrrolo[2,3-d]pyrimidin-2- |
| | amine and (4bS,8aS)-4b,7,7-trimethyl-9-((S)-7-(methylamino)-6,7-dihydro-5H- |
| | cyclopenta[b]pyridin-2-yl)-N-(4-morpholinophenyl)-4b,5,7,8,8a,9- |
| | hexahydropyrano[3′,4′:4,5]pyrrolo[2,3-d]pyrimidin-2-amine |
| 553, 554 | (4bR,8aR)-4b,7,7-trimethyl-9-((R)-7-(methylamino)-6,7-dihydro-5H-cyclopenta[b]pyridin-2-yl)- |
| | N-(4-morpholinophenyl)-4b,5,7,8,8a,9-hexahydropyrano[3′,4′:4,5]pyrrolo[2,3-d]pyrimidin-2- |
| | amine and (4bS,8aS)-4b,7,7-trimethyl-9-((R)-7-(methylamino)-6,7-dihydro-5H- |
| | cyclopenta[b]pyridin-2-yl)-N-(4-morpholinophenyl)-4b,5,7,8,8a,9- |
| | hexahydropyrano[3′,4′:4,5]pyrrolo[2,3-d]pyrimidin-2-amine |
| 555, 556 | (S)-5-methyl-1-(6-((4bS,8aS)-4b,7,7-trimethyl-2-((1-(1-methylpiperidin-4-yl)-1H-pyrazol-4- |
| | yl)amino)-4b,7,8,8a-tetrahydropyrano[3′,4′:4,5]pyrrolo[2,3-d]pyrimidin-9(5H)-yl)pyridin-2- |
| | yl)pyrrolidin-2-one and (S)-5-methyl-1-(6-((4bR,8aR)-4b,7,7-trimethyl-2-((1-(1- |
| | methylpiperidin-4-yl)-1H-pyrazol-4-yl)amino)-4b,7,8,8a-tetrahydropyrano[3′,4′:4,5]pyrrolo[2,3- |
| | d]pyrimidin-9(5H)-yl)pyridin-2-yl)pyrrolidin-2-one |
| 557, 558 | (4bR,8aR)-4b,7,7-trimethyl-9-(6-methyl-4-((R)-1-(methylamino)ethyl)pyridin-2-yl)-N-(4- |
| | morpholinophenyl)-4b,5,7,8,8a,9-hexahydropyrano[3′,4′:4,5]pyrrolo[2,3-d]pyrimidin-2-amine |
| | and (4bS,8aS)-4b,7,7-trimethyl-9-(6-methyl-4-((R)-1-(methylamino)ethyl)pyridin-2-yl)-N-(4- |
| | morpholinophenyl)-4b,5,7,8,8a,9-hexahydropyrano[3′,4′:4,5]pyrrolo[2,3-d]pyrimidin-2-amine |
| 559, 560 | (4bS,8aS)-4b,7,7-trimethyl-9-(6-methyl-4-((S)-1-(methylamino)ethyl)pyridin-2-yl)-N-(4- |
| | morpholinophenyl)-4b,5,7,8,8a,9-hexahydropyrano[3′,4′:4,5]pyrrolo[2,3-d]pyrimidin-2-amine |
| | and (4bR,8aR)-4b,7,7-trimethyl-9-(6-methyl-4-((S)-1-(methylamino)ethyl)pyridin-2-yl)-N-(4- |
| | morpholinophenyl)-4b,5,7,8,8a,9-hexahydropyrano[3′,4′:4,5]pyrrolo[2,3-d]pyrimidin-2-amine |
| 561, 562 | (4bS,8aS)-4b,7,7-trimethyl-N-(4-morpholinophenyl)-9-(5,6,7,8-tetrahydro-1,6-naphthyridin-2- |
| | yl)-4b,5,7,8,8a,9-hexahydropyrano[3′,4′:4,5]pyrrolo[2,3-d]pyrimidin-2-amine and (4bR,8aR)- |
| | 4b,7,7-trimethyl-N-(4-morpholinophenyl)-9-(5,6,7,8-tetrahydro-1,6-naphthyridin-2-yl)- |
| | 4b,5,7,8,8a,9-hexahydropyrano[3′,4′:4,5]pyrrolo[2,3-d]pyrimidin-2-amine |
| 563, 564 | (4bS,8aS)-9-((R)-7-amino-7-methyl-6,7-dihydro-5H-cyclopenta[b]pyridin-2-yl)-4b,7,7- |
| | trimethyl-N-(4-morpholinophenyl)-4b,5,7,8,8a,9-hexahydropyrano[3′,4′:4,5]pyrrolo[2,3- |
| | d]pyrimidin-2-amine and (4bR,8aR)-9-((R)-7-amino-7-methyl-6,7-dihydro-5H- |
| | cyclopenta[b]pyridin-2-yl)-4b,7,7-trimethyl-N-(4-morpholinophenyl)-4b,5,7,8,8a,9- |
| | hexahydropyrano[3′,4′:4,5]pyrrolo[2,3-d]pyrimidin-2-amine |
| 565, 566 | (4bR,8aR)-9-((S)-7-amino-7-methyl-6,7-dihydro-5H-cyclopenta[b]pyridin-2-yl)-4b,7,7- |
| | trimethyl-N-(4-morpholinophenyl)-4b,5,7,8,8a,9-hexahydropyrano[3′,4′:4,5]pyrrolo[2,3- |
| | d]pyrimidin-2-amine and (4bS,8aS)-9-((S)-7-amino-7-methyl-6,7-dihydro-5H- |
| | cyclopenta[b]pyridin-2-yl)-4b,7,7-trimethyl-N-(4-morpholinophenyl)-4b,5,7,8,8a,9- |
| | hexahydropyrano[3′,4′:4,5]pyrrolo[2,3-d]pyrimidin-2-amine |
| 567, 568 | (4bR,8aR)-9-(6-(2-aminopropan-2-yl)pyridin-2-yl)-4b,7,7-trimethyl-N-(4-morpholinophenyl)- |
| | 4b,5,7,8,8a,9-hexahydropyrano[3′,4′:4,5]pyrrolo[2,3-d]pyrimidin-2-amine and (4bS,8aS)-9-(6- |
| | (2-aminopropan-2-yl)pyridin-2-yl)-4b,7,7-trimethyl-N-(4-morpholinophenyl)-4b,5,7,8,8a,9- |
| | hexahydropyrano[3′,4′:4,5]pyrrolo[2,3-d]pyrimidin-2-amine |
| 569, 570 | (4bR,8aR)-4b,7,7-trimethyl-9-((R)-7-methyl-7-(methylamino)-6,7-dihydro-5H- |
| | cyclopenta[b]pyridin-2-yl)-N-(4-morpholinophenyl)-4b,5,7,8,8a,9- |
| | hexahydropyrano[3′,4′:4,5]pyrrolo[2,3-d]pyrimidin-2-amine and (4bS,8aS)-4b,7,7-trimethyl-9- |
| | ((R)-7-methyl-7-(methylamino)-6,7-dihydro-5H-cyclopenta[b]pyridin-2-yl)-N-(4- |
| | morpholinophenyl)-4b,5,7,8,8a,9-hexahydropyrano[3′,4′:4,5]pyrrolo[2,3-d]pyrimidin-2-amine |
| 571 | (4bR,8aR)-4b,7,7-trimethyl-9-((S)-7-methyl-7-(methylamino)-6,7-dihydro-5H- |
| | cyclopenta[b]pyridin-2-yl)-N-(4-morpholinophenyl)-4b,5,7,8,8a,9- |
| | hexahydropyrano[3′,4′:4,5]pyrrolo[2,3-d]pyrimidin-2-amine |
| 572, 573 | (4bR,8aR)-4b-methyl-9-(6-((methylamino)methyl)pyridin-2-yl)-N-(4-morpholinophenyl)- |
| | 4b,5,7,8,8a,9-hexahydropyrano[3′,4′:4,5]pyrrolo[2,3-d]pyrimidin-2-amine and (4bS,8aS)-4b- |
| | methyl-9-(6-((methylamino)methyl)pyridin-2-yl)-N-(4-morpholinophenyl)-4b,5,7,8,8a,9- |
| | hexahydropyrano[3′,4′:4,5]pyrrolo[2,3-d]pyrimidin-2-amine |
| 574, 575 | (4bS,8aS)-4b,7,7-trimethyl-N-(3-methyl-4-(1-methylpiperidin-4-yl)phenyl)-9-(6-(1- |
| | (methylamino)cyclopropyl)pyridin-2-yl)-4b,5,7,8,8a,9-hexahydropyrano[3′,4′:4,5]pyrrolo[2,3- |
| | d]pyrimidin-2-amine and (4bR,8aR)-4b,7,7-trimethyl-N-(3-methyl-4-(1-methylpiperidin-4- |
| | yl)phenyl)-9-(6-(1-(methylamino)cyclopropyl)pyridin-2-yl)-4b,5,7,8,8a,9- |
| | hexahydropyrano[3′,4′:4,5]pyrrolo[2,3-d]pyrimidin-2-amine |
| 576, 577 | 3-(6-((4bS,8aS)-4b,7,7-trimethyl-2-((4-(4-methylpiperazin-1-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | 3-(6-((4bR,8aR)-4b,7,7-trimethyl-2-((4-(4-methylpiperazin-1-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 578, 579 | (4bS,8aS)-N-(4-((1R,4R)-2-oxa-5-azabicyclo[2.2.1]heptan-5-yl)phenyl)-4b,7,7-trimethyl-9-(6- |
| | ((S)-1-(methylamino)ethyl)pyridin-2-yl)-4b,5,7,8,8a,9-hexahydropyrano[3′,4′:4,5]pyrrolo[2,3- |
| | d]pyrimidin-2-amine and (4bR,8aR)-N-(4-((1R,4R)-2-oxa-5-azabicyclo[2.2.1]heptan-5- |
| | yl)phenyl)-4b,7,7-trimethyl-9-(6-((S)-1-(methylamino)ethyl)pyridin-2-yl)-4b,5,7,8,8a,9- |
| | hexahydropyrano[3′,4′:4,5]pyrrolo[2,3-d]pyrimidin-2-amine |
| 580, 581 | (4bS,8aS)-N-(4-((1S,4S)-2-oxa-5-azabicyclo[2.2.1]heptan-5-yl)phenyl)-4b,7,7-trimethyl-9-(6- |
| | ((S)-1-(methylamino)ethyl)pyridin-2-yl)-4b,5,7,8,8a,9-hexahydropyrano[3′,4′:4,5]pyrrolo[2,3- |
| | d]pyrimidin-2-amine and (4bR,8aR)-N-(4-((1S,4S)-2-oxa-5-azabicyclo[2.2.1]heptan-5- |
| | yl)phenyl)-4b,7,7-trimethyl-9-(6-((S)-1-(methylamino)ethyl)pyridin-2-yl)-4b,5,7,8,8a,9- |
| | hexahydropyrano[3′,4′:4,5]pyrrolo[2,3-d]pyrimidin-2-amine |
| 582, 583 | (S)-5-methyl-1-(6-((4bS,8aS)-4b,7,7-trimethyl-2-((4-(4-methylpiperazin-1-yl)phenyl)amino)- |
| | 4b,7,8,8a-tetrahydropyrano[3′,4′:4,5]pyrrolo[2,3-d]pyrimidin-9(5H)-yl)pyridin-2-yl)pyrrolidin-2- |
| | one and (S)-5-methyl-1-(6-((4bR,8aR)-4b,7,7-trimethyl-2-((4-(4-methylpiperazin-1- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)pyrrolidin-2-one |
| 584, 585 | N,N-dimethyl-6-((4bS,8aS)-4b,7,7-trimethyl-2-((3-methyl-4-(1-methylpiperidin-4- |
| | yl)phenyl)amino)-4b,7,8,8a-tetrahydropyrano[3′,4′:4,5]pyrrolo[2,3-d]pyrimidin-9(5H)- |
| | yl)picolinamide and N,N-dimethyl-6-((4bR,8aR)-4b,7,7-trimethyl-2-((3-methyl-4-(1- |
| | methylpiperidin-4-yl)phenyl)amino)-4b,7,8,8a-tetrahydropyrano[3′,4′:4,5]pyrrolo[2,3- |
| | d]pyrimidin-9(5H)-yl)picolinamide |
| 586, 587 | 1-(6-((4bS,8aS)-4b,7,7-trimethyl-2-((4-(4-methylpiperazin-1-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one and 1- |
| | (6-((4bR,8aR)-4b,7,7-trimethyl-2-((4-(4-methylpiperazin-1-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 588, 589 | (R)-4-methyl-3-(6-((4bS,8aS)-4b,7,7-trimethyl-2-((4-(4-methylpiperazin-1-yl)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and (R)-4-methyl-3-(6-((4bR,8aR)-4b,7,7-trimethyl-2-((4-(4-methylpiperazin-1- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one |
| 590, 591 | 3-(6-((4bS,8aS)-4b,7,7-trimethyl-2-((4-(1-methylpiperidin-4-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | 3-(6-((4bR,8aR)-4b,7,7-trimethyl-2-((4-(1-methylpiperidin-4-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 592, 593 | 3-(6-((4bS,8aS)-4b,7,7-trimethyl-2-((4-((1R,4R)-5-methyl-2,5-diazabicyclo[2.2.1]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one and 3-(6-((4bR,8aR)-4b,7,7-trimethyl-2-((4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 594, 595 | 3-(6-((4bS,8aS)-4b,7,7-trimethyl-2-((4-((1S,4S)-5-methyl-2,5-diazabicyclo[2.2.1]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one and 3-(6-((4bR,8aR)-46,7,7-trimethyl-2-((4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 596, 597 | 3-(6-((4bS,8aS)-4b,7,7-trimethyl-2-((3-methyl-4-(1-methylpiperidin-4-yl)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and 3-(6-((4bR,8aR)-4b,7,7-trimethyl-2-((3-methyl-4-(1-methylpiperidin-4- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one |
| 598, 599 | (S)-5-methyl-3-(6-((4bS,8aS)-4b,7,7-trimethyl-2-((4-(4-methylpiperazin-1-yl)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and (S)-5-methyl-3-(6-((4bR,8aR)-4b,7,7-trimethyl-2-((4-(4-methylpiperazin-1- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one |
| 600, 601 | 3-(6-((4bS,8aS)-4b,7,7-trimethyl-2-((4-(piperazin-1-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | 3-(6-((4bR,8aR)-4b,7,7-trimethyl-2-((4-(piperazin-1-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 602, 603 | 3-(6-((4bR,8aR)-4b,7,7-trimethyl-2-((4-((R)-1-methylpyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | 3-(6-((4bS,8aS)-4b,7,7-trimethyl-2-((4-((R)-1-methylpyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 604, 605 | 3-(6-((4bS,8aS)-4b,7,7-trimethyl-2-((4-((S)-1-methylpyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | 3-(6-((4bR,8aR)-4b,7,7-trimethyl-2-((4-((S)-1-methylpyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 606, 607 | 3-(6-((4bS,8aS)-4b,7,7-trimethyl-2-((4-((S)-pyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | 3-(6-((4bR,8aR)-4b,7,7-trimethyl-2-((4-((S)-pyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 608, 609 | 3-(6-((4bR,8aR)-4b,7,7-trimethyl-2-((4-((R)-pyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | 3-(6-((4bS,8aS)-4b,7,7-trimethyl-2-((4-((R)-pyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 610, 611 | 2-(4-(((4bS,8aS)-9-(6-((dimethylamino)methyl)pyridin-2-yl)-4b,7,7-trimethyl-4b,5,7,8,8a,9- |
| | hexahydropyrano[3′,4′:4,5]pyrrolo[2,3-d]pyrimidin-2-yl)amino)phenyl)-2-methylpropanoic acid |
| | and 2-(4-(((4bR,8aR)-9-(6-((dimethylamino)methyl)pyridin-2-yl)-4b,7,7-trimethyl- |
| | 4b,5,7,8,8a,9-hexahydropyrano[3′,4′:4,5]pyrrolo[2,3-d]pyrimidin-2-yl)amino)phenyl)-2- |
| | methylpropanoic acid |
| 612, 613 | 3-(6-((4bS,8aS)-4b,7,7-trimethyl-2-((4-(((S)-pyrrolidin-3-yl)oxy)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | 3-(6-((4bR,8aR)-4b,7,7-trimethyl-2-((4-(((S)-pyrrolidin-3-yl)oxy)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 614, 615 | 3-(6-((4bS,8aS)-4b,7,7-trimethyl-2-((4-(((S)-1-methylpyrrolidin-3-yl)oxy)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one |
| | and 3-(6-((4bR,8aR)-4b,7,7-trimethyl-2-((4-(((S)-1-methylpyrrolidin-3-yl)oxy)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin- |
| | 2-one |
| 616, 617 | 1-(6-((4bR,8aR)-4b,7,7-trimethyl-2-((4-(((R)-pyrrolidin-3-yl)oxy)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one and 1- |
| | (6-((4bS,8aS)-4b,7,7-trimethyl-2-((4-(((R)-pyrrolidin-3-yl)oxy)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 618, 619 | 1-(6-((4bR,8aR)-4b,7,7-trimethyl-2-((4-(((S)-1-methylpyrrolidin-3-yl)oxy)phenyl)amino)- |
| | 4b,7,8,8a-tetrahydropyrano[3′,4′:4,5]pyrrolo[2,3-d]pyrimidin-9(5H)-yl)pyridin-2-yl)pyrrolidin-2- |
| | one and 1-(6-((4bS,8aS)-4b,7,7-trimethyl-2-((4-(((S)-1-methylpyrrolidin-3- |
| | yl)oxy)phenyl)amino)-4b,7,8,8a-tetrahydropyrano[3′,4′:4,5]pyrrolo[2,3-d]pyrimidin-9(5H)- |
| | yl)pyridin-2-yl)pyrrolidin-2-one |
| 620, 621 | 1-(6-((4bR,8aR)-4b,7,7-trimethyl-2-((4-(((S)-pyrrolidin-3-yl)oxy)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one and 1- |
| | (6-((4bS,8aS)-4b,7,7-trimethyl-2-((4-(((S)-pyrrolidin-3-yl)oxy)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 622, 623 | 3-(6-((4bR,8aR)-4b,7,7-trimethyl-2-((4-(((R)-pyrrolidin-3-yl)oxy)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | 3-(6-((4bS,8aS)-4b,7,7-trimethyl-2-((4-(((R)-pyrrolidin-3-yl)oxy)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 624, 625 | 3-(6-((4bR,8aR)-4b,7,7-trimethyl-2-((4-(((R)-1-methylpyrrolidin-3-yl)oxy)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and 3-(6-((4bS,8aS)-4b,7,7-trimethyl-2-((4-(((R)-1-methylpyrrolidin-3- |
| | yl)oxy)phenyl)amino)-4b,7,8,8a-tetrahydropyrano[3′,4′:4,5]pyrrolo[2,3-d]pyrimidin-9(5H)- |
| | yl)pyridin-2-yl)oxazolidin-2-one |
| 626, 627 | 1-(6-((4bR,8aR)-4b,7,7-trimethyl-2-((4-(((R)-1-methylpyrrolidin-3-yl)oxy)phenyl)amino)- |
| | 4b,7,8,8a-tetrahydropyrano[3′,4′:4,5]pyrrolo[2,3-d]pyrimidin-9(5H)-yl)pyridin-2-yl)pyrrolidin-2- |
| | one and 1-(6-((4bS,8aS)-4b,7,7-trimethyl-2-((4-(((R)-1-methylpyrrolidin-3- |
| | yl)oxy)phenyl)amino)-4b,7,8,8a-tetrahydropyrano[3′,4′:4,5]pyrrolo[2,3-d]pyrimidin-9(5H)- |
| | yl)pyridin-2-yl)pyrrolidin-2-one |
| 628, 629 | 1-(6-((4bS,8aS)-4b,7,7-trimethyl-2-((4-((1S,4S)-5-methyl-2,5-diazabicyclo[2.2.1]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)pyrrolidin-2-one and 1-(6-((4bR,8aR)-4b,7,7-trimethyl-2-((4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 630, 631 | 1-(6-((4bS,8aS)-4b,7,7-trimethyl-2-((4-((1R,4R)-5-methyl-2,5-diazabicyclo[2.2.1]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)pyrrolidin-2-one and 1-(6-((4bR,8aR)-4b,7,7-trimethyl-2-((4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 632, 633 | 1-(6-((4bR,8aR)-4b,7,7-trimethyl-2-((4-((S)-pyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one and 1- |
| | (6-((4bS,8aS)-4b,7,7-trimethyl-2-((4-((S)-pyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 634, 635 | 1-(6-((4bR,8aR)-4b,7,7-trimethyl-2-((4-((R)-1-methylpyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one and 1- |
| | (6-((4bS,8aS)-4b,7,7-trimethyl-2-((4-((R)-1-methylpyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 636, 637 | 1-(6-((4bR,8aR)-4b,7,7-trimethyl-2-((4-((R)-pyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one and 1- |
| | (6-((4bS,8aS)-4b,7,7-trimethyl-2-((4-((R)-pyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 638, 639 | 1-(6-((4bR,8aR)-2-((3-fluoro-4-((1R,4R)-5-methyl-2,5-diazabicyclo[2.2.1]heptan-2- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)pyrrolidin-2-one and 1-(6-((4bS,8aS)-2-((3-fluoro-4-((1R,4R)- |
| | 5-methyl-2,5-diazabicyclo[2.2.1]heptan-2-yl)phenyl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)pyrrolidin-2-one |
| 640, 641 | 1-(6-((4bR,8aR)-2-((3-fluoro-4-((1S,4S)-5-methyl-2,5-diazabicyclo[2.2.1]heptan-2- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)pyrrolidin-2-one and 1-(6-((4bS,8aS)-2-((3-fluoro-4-((1S,4S)- |
| | 5-methyl-2,5-diazabicyclo[2.2.1]heptan-2-yl)phenyl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)pyrrolidin-2-one |
| 642, 643 | 1-(6-((4bR,8aR)-4b,7,7-trimethyl-2-((4-((S)-1-methylpyrrolidin-3-yl)phenyl)amino)-46,7,8,8a- |
| | tetrahydropyrano[3′,4′:4,5]pyrrolo[2,3-d]pyrimidin-9(5H)-yl)pyridin-2-yl)pyrrolidin-2-one and 1- |
| | (6-((4bS,8aS)-4b,7,7-trimethyl-2-((4-((S)-1-methylpyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 644, 645 | 1-(6-((4bR,8aR)-4b,7,7-trimethyl-2-((4-((1S,4S)-5-methyl-2,5-diazabicyclo[2.2.1]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)cyclopropan-1-ol and 1-(6-((4bS,8aS)-4b,7,7-trimethyl-2-((4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)cyclopropan-1-ol |
| 646, 647 | 1-(6-((4bR,8aR)-4b,7,7-trimethyl-2-((4-((1R,4R)-5-methyl-2,5-diazabicyclo[2.2.1]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)cyclopropan-1-ol and 1-(6-((4bS,8aS)-4b,7,7-trimethyl-2-((4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)cyclopropan-1-ol |
| 650, 651 | (S)-5-methyl-1-(6-((4bS,8aS)-4b,7,7-trimethyl-2-((4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one and |
| | (S)-5-methyl-1-(6-((4bR,8aR)-4b,7,7-trimethyl-2-((4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 652, 653 | 1-(6-((4bR,8aR)-4b,7,7-trimethyl-2-((3-methyl-4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one and 1- |
| | (6-((4bS,8aS)-4b,7,7-trimethyl-2-((3-methyl-4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 654, 655 | 3-(6-((4bR,8aR)-4b,7,7-trimethyl-2-((3-methyl-4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | 3-(6-((4bS,8aS)-4b,7,7-trimethyl-2-((3-methyl-4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 656, 657 | (S)-5-methyl-1-(6-((4bS,8aS)-4b,7,7-trimethyl-2-((4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one and |
| | (S)-5-methyl-1-(6-((4bR,8aR)-4b,7,7-trimethyl-2-((4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 658, 659 | 1-(6-((4bR,8aR)-4b,7,7-trimethyl-2-((3-methyl-4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one and 1- |
| | (6-((4bS,8aS)-4b,7,7-trimethyl-2-((3-methyl-4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 660, 661 | 3-(6-((4bR,8aR)-4b,7,7-trimethyl-2-((3-methyl-4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | 3-(6-((4bS,8aS)-4b,7,7-trimethyl-2-((3-methyl-4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 662, 663 | 1-(6-((4bR,8aR)-2-((4-(azetidin-3-yl)phenyl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)pyrrolidin-2-one and 1- |
| | (6-((4bS,8aS)-2-((4-(azetidin-3-yl)phenyl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)pyrrolidin-2-one |
| 664, 665 | 3-(6-((4bR,8aR)-4b,7,7-trimethyl-2-((4-((S)-3-methylpyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | 3-(6-((4bS,8aS)-46,7,7-trimethyl-2-((4-((S)-3-methylpyrrolidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 666, 667 | 3-(6-((4bR,8aR)-2-((4-(azetidin-3-yl)phenyl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)oxazolidin-2-one and |
| | 3-(6-((4bS,8aS)-2-((4-(azetidin-3-yl)phenyl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)oxazolidin-2-one |
| 668, 669 | 3-(6-((4bR,8aR)-2-((3-fluoro-4-((1R,4R)-5-methyl-2,5-diazabicyclo[2.2.1]heptan-2- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)oxazolidin-2-one and 3-(6-((4bS,8aS)-2-((3-fluoro-4- |
| | ((1R,4R)-5-methyl-2,5-diazabicyclo[2.2.1]heptan-2-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2-yl)oxazolidin-2- |
| | one |
| 670, 671 | 3-(6-((4bR,8aR)-2-((4-((S)-1,3-dimethylpyrrolidin-3-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and 3-(6-((4bS,8aS)-2-((4-((S)-1,3-dimethylpyrrolidin-3-yl)phenyl)amino)-4b,7,7- |
| | trimethyl-4b,7,8,8a-tetrahydropyrano[3′,4′:4,5]pyrrolo[2,3-d]pyrimidin-9(5H)-yl)pyridin-2- |
| | yl)oxazolidin-2-one |
| 672, 673 | 3-(6-((4bR,8aR)-2-((4-((3S,4S)-4-fluoropyrrolidin-3-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and 3-(6-((4bS,8aS)-2-((4-((3S,4S)-4-fluoropyrrolidin-3-yl)phenyl)amino)-4b,7,7- |
| | trimethyl-4b,7,8,8a-tetrahydropyrano[3′,4′:4,5]pyrrolo[2,3-d]pyrimidin-9(5H)-yl)pyridin-2- |
| | yl)oxazolidin-2-one |
| 674, 675 | 3-(6-((4bS,8aS)-2-((4-((3R,4R)-4-fluoropyrrolidin-3-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and 3-(6-((4bR,8aR)-2-((4-((3R,4R)-4-fluoropyrrolidin-3-yl)phenyl)amino)-4b,7,7- |
| | trimethyl-4b,7,8,8a-tetrahydropyrano[3′,4′:4,5]pyrrolo[2,3-d]pyrimidin-9(5H)-yl)pyridin-2- |
| | yl)oxazolidin-2-one |
| 676, 677 | 3-(6-((4bR,8aR)-2-((3-fluoro-4-((1S,4S)-5-methyl-2,5-diazabicyclo[2.2.1]heptan-2- |
| | yl)phenyl)amino)-4b,7,7-trimethyl-4b,7,8,8a-tetrahydropyrano[3′,4′:4,5]pyrrolo[2,3- |
| | d]pyrimidin-9(5H)-yl)pyridin-2-yl)oxazolidin-2-one and 3-(6-((4bS,8aS)-2-((3-fluoro-4- |
| | ((1S,4S)-5-methyl-2,5-diazabicyclo[2.2.1]heptan-2-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2-yl)oxazolidin-2- |
| | one |
| 680, 681 | 3-(6-((4bR,8aR)-2-((4-((R)-4,4-difluoropyrrolidin-3-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and 3-(6-((4bS,8aS)-2-((4-((R)-4,4-difluoropyrrolidin-3-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2-yl)oxazolidin-2- |
| | one |
| 682, 683 | 3-(6-((4bR,8aR)-2-((4-((S)-4,4-difluoropyrrolidin-3-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and 3-(6-((4bS,8aS)-2-((4-((S)-4,4-difluoropyrrolidin-3-yl)phenyl)amino)-4b,7,7-trimethyl- |
| | 4b,7,8,8a-tetrahydropyrano[3′,4′:4,5]pyrrolo[2,3-d]pyrimidin-9(5H)-yl)pyridin-2-yl)oxazolidin-2- |
| | one |
| 684, 685 | 3-(6-((4bR,8aR)-4b,7,7-trimethyl-2-((4-((3aS,6aS)-1-methylhexahydropyrrolo[3,4-b]pyrrol- |
| | 5(1H)-yl)phenyl)amino)-4b,7,8,8a-tetrahydropyrano[3′,4′:4,5]pyrrolo[2,3-d]pyrimidin-9(5H)- |
| | yl)pyridin-2-yl)oxazolidin-2-one and 3-(6-((4bS,8aS)-4b,7,7-trimethyl-2-((4-((3aS,6aS)-1- |
| | methylhexahydropyrrolo[3,4-b]pyrrol-5(1H)-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 686, 687 | 1-(6-((4bR,8aR)-4b,7,7-trimethyl-2-((4-((3aR,6aR)-1-methylhexahydropyrrolo[3,4-b]pyrrol- |
| | 5(1H)-yl)phenyl)amino)-4b,7,8,8a-tetrahydropyrano[3′,4′:4,5]pyrrolo[2,3-d]pyrimidin-9(5H)- |
| | yl)pyridin-2-yl)pyrrolidin-2-one and 1-(6-((4bS,8aS)-4b,7,7-trimethyl-2-((4-((3aR,6aR)-1- |
| | methylhexahydropyrrolo[3,4-b]pyrrol-5(1H)-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 688, 689 | 1-(6-((4bR,8aR)-4b,7,7-trimethyl-2-((4-((3aS,6aS)-1-methylhexahydropyrrolo[3,4-b]pyrrol- |
| | 5(1H)-yl)phenyl)amino)-4b,7,8,8a-tetrahydropyrano[3′,4′:4,5]pyrrolo[2,3-d]pyrimidin-9(5H)- |
| | yl)pyridin-2-yl)pyrrolidin-2-one and 1-(6-((4bS,8aS)-4b,7,7-trimethyl-2-((4-((3aS,6aS)-1- |
| | methylhexahydropyrrolo[3,4-b]pyrrol-5(1H)-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 690, 691 | 1-(6-((4bR,8aR)-4b,7,7-trimethyl-2-((4-(6-methyl-2,6-diazaspiro[3.3]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)pyrrolidin-2-one and 1-(6-((4bS,8aS)-4b,7,7-trimethyl-2-((4-(6-methyl-2,6- |
| | diazaspiro[3.3]heptan-2-yl)phenyl)amino)-4b,7,8,8a-tetrahydropyrano[3′,4′:4,5]pyrrolo[2,3- |
| | d]pyrimidin-9(5H)-yl)pyridin-2-yl)pyrrolidin-2-one |
| 692, 693 | 3-(6-((4bR,8aR)-4b,7,7-trimethyl-2-((4-((3aR,6aS)-5-methylhexahydropyrrolo[3,4-c]pyrrol- |
| | 2(1H)-yl)phenyl)amino)-4b,7,8,8a-tetrahydropyrano[3′,4′:4,5]pyrrolo[2,3-d]pyrimidin-9(5H)- |
| | yl)pyridin-2-yl)oxazolidin-2-one and 3-(6-((4bS,8aS)-46,7,7-trimethyl-2-((4-((3aR,6aS)-5- |
| | methylhexahydropyrrolo[3,4-c]pyrrol-2(1H)-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 694, 695 | 1-(6-((4bR,8aR)-4b,7,7-trimethyl-2-((4-((3aR,6aS)-5-methylhexahydropyrrolo[3,4-c]pyrrol- |
| | 2(1H)-yl)phenyl)amino)-4b,7,8,8a-tetrahydropyrano[3′,4′:4,5]pyrrolo[2,3-d]pyrimidin-9(5H)- |
| | yl)pyridin-2-yl)pyrrolidin-2-one and 1-(6-((4bS,8aS)-4b,7,7-trimethyl-2-((4-((3aR,6aS)-5- |
| | methylhexahydropyrrolo[3,4-c]pyrrol-2(1H)-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 696, 697 | 1-(6-((4bR,8aR)-4b,7,7-trimethyl-2-((4-(1-methylazetidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one and 1- |
| | (6-((4bS,8aS)-4b,7,7-trimethyl-2-((4-(1-methylazetidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 698, 699 | 3-(6-((4bR,8aR)-4b,7,7-trimethyl-2-((4-(1-methylazetidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | 3-(6-((4bS,8aS)-4b,7,7-trimethyl-2-((4-(1-methylazetidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 700, 701 | 3-(6-((4bR,8aR)-4b,7,7-trimethyl-2-((4-((1R,5S)-6-methyl-3,6-diazabicyclo[3.1.1]heptan-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one and 3-(6-((4bS,8aS)-4b,7,7-trimethyl-2-((4-((1R,5S)-6-methyl-3,6- |
| | diazabicyclo[3.1.1]heptan-3-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 702 | (S)-4-methyl-3-(6-((4bR,8aR)-4b-methyl-2-((4-(4-methylpiperazin-1-yl)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one |
| 703, 704 | 1-(6-((4bR,8aR)-4b,7,7-trimethyl-2-((4-((1R,5S)-6-methyl-3,6-diazabicyclo[3.1.1]heptan-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)pyrrolidin-2-one and 1-(6-((4bS,8aS)-4b,7,7-trimethyl-2-((4-((1R,5S)-6-methyl-3,6- |
| | diazabicyclo[3.1.1]heptan-3-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 705, 706 | 3-(6-((4bR,8aR)-4b,7,7-trimethyl-2-((4-((3aR,6aR)-1-methylhexahydropyrrolo[3,4-b]pyrrol- |
| | 5(1H)-yl)phenyl)amino)-4b,7,8,8a-tetrahydropyrano[3′,4′:4,5]pyrrolo[2,3-d]pyrimidin-9(5H)- |
| | yl)pyridin-2-yl)oxazolidin-2-one and 3-(6-((4bS,8aS)-4b,7,7-trimethyl-2-((4-((3aR,6aR)-1- |
| | methylhexahydropyrrolo[3,4-b]pyrrol-5(1H)-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 707, 708 | 3-(6-((4bR,8aR)-4b,7,7-trimethyl-2-((4-(6-methyl-2,6-diazaspiro[3.3]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one and 3-(6-((4bS,8aS)-4b,7,7-trimethyl-2-((4-(6-methyl-2,6- |
| | diazaspiro[3.3]heptan-2-yl)phenyl)amino)-4b,7,8,8a-tetrahydropyrano[3′,4′:4,5]pyrrolo[2,3- |
| | d]pyrimidin-9(5H)-yl)pyridin-2-yl)oxazolidin-2-one |
| 709, 710 | dimethyl((3-((4bS,8aS)-4b,7,7-trimethyl-2-((6-(1-methylpiperidin-4-yl)pyridin-3-yl)amino)- |
| | 4b,7,8,8a-tetrahydropyrano[3′,4′:4,5]pyrrolo[2,3-d]pyrimidin-9(5H)-yl)phenyl)imino)-16- |
| | sulfanone and dimethyl((3-((4bR,8aR)-4b,7,7-trimethyl-2-((6-(1-methylpiperidin-4-yl)pyridin-3- |
| | yl)amino)-4b,7,8,8a-tetrahydropyrano[3′,4′:4,5]pyrrolo[2,3-d]pyrimidin-9(5H)-yl)phenyl)imino)- |
| | 16-sulfanone |
| 711, 712 | dimethyl((3-((4bS,8aS)-4b,7,7-trimethyl-2-((4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)phenyl)imino)-16-sulfanone and |
| | dimethyl((3-((4bR,8aR)-4b,7,7-trimethyl-2-((4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)phenyl)imino)-16-sulfanone |
| 713, 714 | dimethyl((3-((4bR,8aR)-4b,7,7-trimethyl-2-((4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)phenyl)imino)-16-sulfanone and |
| | dimethyl((3-((4bS,8aS)-4b,7,7-trimethyl-2-((4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)phenyl)imino)-16-sulfanone |
| 715, 716 | dimethyl((3-((4bS,8aS)-4b,7,7-trimethyl-2-((1-(1-methylpiperidin-4-yl)-1H-pyrazol-4-yl)amino)- |
| | 4b,7,8,8a-tetrahydropyrano[3′,4′:4,5]pyrrolo[2,3-d]pyrimidin-9(5H)-yl)phenyl)imino)-16- |
| | sulfanone and dimethyl((3-((4bR,8aR)-4b,7,7-trimethyl-2-((1-(1-methylpiperidin-4-yl)-1H- |
| | pyrazol-4-yl)amino)-4b,7,8,8a-tetrahydropyrano[3′,4′:4,5]pyrrolo[2,3-d]pyrimidin-9(5H)- |
| | yl)phenyl)imino)-16-sulfanone |
| 717 | (4bR,8aR)-4b,7,7-trimethyl-9-(1-methyl-1H-pyrazol-3-yl)-N-(4-(4-methylpiperazin-1- |
| | yl)phenyl)-4b,5,7,8,8a,9-hexahydropyrano[3′,4′:4,5]pyrrolo[2,3-d]pyrimidin-2-amine |
| 719 | (4bR,8aR)-4b,7,7-trimethyl-N-(4-(4-methylpiperazin-1-yl)phenyl)-9-(1-(trifluoromethyl)-1H- |
| | pyrazol-3-yl)-4b,5,7,8,8a,9-hexahydropyrano[3′,4′:4,5]pyrrolo[2,3-d]pyrimidin-2-amine |
| 720 | (4bR,8aR)-9-(1-(difluoromethyl)-1H-pyrazol-3-yl)-4b,7,7-trimethyl-N-(4-(4-methylpiperazin-1- |
| | yl)phenyl)-4b,5,7,8,8a,9-hexahydropyrano[3′,4′:4,5]pyrrolo[2,3-d]pyrimidin-2-amine |
| 721 | (4bR,8aR)-9-(1-isopropyl-1H-pyrazol-3-yl)-4b,7,7-trimethyl-N-(4-(4-methylpiperazin-1- |
| | yl)phenyl)-4b,5,7,8,8a,9-hexahydropyrano[3′,4′:4,5]pyrrolo[2,3-d]pyrimidin-2-amine |
| 722 | (4bR,8aR)-4b,7,7-trimethyl-N-(4-(4-methylpiperazin-1-yl)phenyl)-9-(pyridin-2-yl)- |
| | 4b,5,7,8,8a,9-hexahydropyrano[3′,4′:4,5]pyrrolo[2,3-d]pyrimidin-2-amine |
| 723 | dimethyl((6-((4bR,8aR)-4b,7,7-trimethyl-2-(methylamino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 725 | (S)-5-methyl-1-(6-((4bR,8aR)-4b,7,7-trimethyl-2-((4-(6-methyl-2,6-diazaspiro[3.3]heptan-2- |
| | yl)phenyl)amino)-4b,7,8,8a-tetrahydropyrano[3′,4′:4,5]pyrrolo[2,3-d]pyrimidin-9(5H)-yl) |
| | pyridin-2-yl)pyrrolidin-2-one |
| 726 | (4bR,8aR)-4b,7,7-trimethyl-N-(4-(4-methylpiperazin-1-yl)phenyl)-9-(thiazol-2-yl)- |
| | 4b,5,7,8,8a,9-hexahydropyrano[3′,4′:4,5]pyrrolo[2,3-d]pyrimidin-2-amine |
| 727 | (S)-5-methyl-1-(6-((4bR,8aR)-4b,7,7-trimethyl-2-((4-(1-methylazetidin-3-yl)phenyl)amino)- |
| | 4b,7,8,8a-tetrahydropyrano[3′,4′:4,5]pyrrolo[2,3-d]pyrimidin-9(5H)-yl)pyridin-2-yl)pyrrolidin- |
| | 2-one |
| 728 | 4,4-dimethyl-3-(6-((4bR,8aR)-4b,7,7-trimethyl-2-((4-(4-methylpiperazin-1-yl)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin- |
| | 2-one |
| 729 | 5,5-dimethyl-1-(6-((4bR,8aR)-4b,7,7-trimethyl-2-((4-(4-methylpiperazin-1-yl)phenyl)amino)- |
| | 4b,7,8,8a-tetrahydropyrano[3′,4′:4,5]pyrrolo[2,3-d]pyrimidin-9(5H)-yl)pyridin-2-yl)pyrrolidin- |
| | 2-one |
| 730 | (S)-5-methyl-1-(6-((4bR,8aR)-4b,7,7-trimethyl-2-((4-((methylamino)methyl)phenyl)amino)- |
| | 4b,7,8,8a-tetrahydropyrano[3′,4′:4,5]pyrrolo[2,3-d]pyrimidin-9(5H)-yl)pyridin-2-yl)pyrrolidin- |
| | 2-one |
| 731, 732 | (4bR,8aR)-4b,7,7-trimethyl-9-(6-((S)-morpholin-3-yl)pyridin-2-yl)-N-(4-morpholinophenyl)- |
| | 4b,5,7,8,8a,9-hexahydropyrano[3′,4′:4,5]pyrrolo[2,3-d]pyrimidin-2-amine and (4bS,8aS)- |
| | 4b,7,7-trimethyl-9-(6-((S)-morpholin-3-yl)pyridin-2-yl)-N-(4-morpholinophenyl)-4b,5,7,8,8a,9- |
| | hexahydropyrano[3′,4′:4,5]pyrrolo[2,3-d]pyrimidin-2-amine |
| 733, 734 | (4bS,8aS)-4b,7,7-trimethyl-9-(6-((R)-morpholin-3-yl)pyridin-2-yl)-N-(4-morpholinophenyl)- |
| | 4b,5,7,8,8a,9-hexahydropyrano[3′,4′:4,5]pyrrolo[2,3-d]pyrimidin-2-amine and (4bR,8aR)- |
| | 4b,7,7-trimethyl-9-(6-((R)-morpholin-3-yl)pyridin-2-yl)-N-(4-morpholinophenyl)-4b,5,7,8,8a,9- |
| | hexahydropyrano[3′,4′:4,5]pyrrolo[2,3-d]pyrimidin-2-amine |
| 735, 736 | (4bR,8aR)-4b,7,7-trimethyl-9-(6-((S)-morpholin-2-yl)pyridin-2-yl)-N-(4-morpholinophenyl)- |
| | 4b,5,7,8,8a,9-hexahydropyrano[3′,4′:4,5]pyrrolo[2,3-d]pyrimidin-2-amine and (4bS,8aS)- |
| | 4b,7,7-trimethyl-9-(6-((S)-morpholin-2-yl)pyridin-2-yl)-N-(4-morpholinophenyl)-4b,5,7,8,8a,9- |
| | hexahydropyrano[3′,4′:4,5]pyrrolo[2,3-d]pyrimidin-2-amine |
| 737, 738 | (4bR,8aR)-4b,7,7-trimethyl-9-(6-((R)-morpholin-2-yl)pyridin-2-yl)-N-(4-morpholinophenyl)- |
| | 4b,5,7,8,8a,9-hexahydropyrano[3′,4′:4,5]pyrrolo[2,3-d]pyrimidin-2-amine and (4bS,8aS)- |
| | 4b,7,7-trimethyl-9-(6-((R)-morpholin-2-yl)pyridin-2-yl)-N-(4-morpholinophenyl)-4b,5,7,8,8a,9- |
| | hexahydropyrano[3′,4′:4,5]pyrrolo[2,3-d]pyrimidin-2-amine |
| 739 | (4bR,8aR)-9-(1-cyclopropyl-1H-pyrazol-3-yl)-4b,7,7-trimethyl-N-(4-(4-methylpiperazin-1- |
| | yl)phenyl)-4b,5,7,8,8a,9-hexahydropyrano[3′,4′:4,5]pyrrolo[2,3-d]pyrimidin-2-amine |
| 742 | (4bR,8aR)-4b,7,7-trimethyl-N-(4-(4-methylpiperazin-1-yl)phenyl)-9-(1-(oxetan-3-yl)-1H- |
| | pyrazol-3-yl)-4b,5,7,8,8a,9-hexahydropyrano[3′,4′:4,5]pyrrolo[2,3-d]pyrimidin-2-amine |
| 744 | (4bR,8aR)-9-(1-ethyl-1H-pyrazol-3-yl)-4b,7,7-trimethyl-N-(4-(4-methylpiperazin-1-yl)phenyl)- |
| | 4b,5,7,8,8a,9-hexahydropyrano[3′,4′:4,5]pyrrolo[2,3-d]pyrimidin-2-amine |
| 746 | ((6-((4bR,8aR)-2-amino-4b,7,7-trimethyl-4b,7,8,8a-tetrahydropyrano[3′,4′:4,5]pyrrolo[2,3- |
| | d]pyrimidin-9(5H)-yl)pyridin-2-yl)imino)dimethyl-16-sulfanone |
| 748 | (S)-5-methyl-1-(6-((4bR,8aR)-4b,7,7-trimethyl-2-((4-((1R,5S)-6-methyl-3,6- |
| | diazabicyclo[3.1.1]heptan-3-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 749 | (4bR,8aR)-4b,7,7-trimethyl-N-(4-(4-methylpiperazin-1-yl)phenyl)-9-(6-(methylsulfonyl)pyridin- |
| | 2-yl)-4b,5,7,8,8a,9-hexahydropyrano[3′,4′:4,5]pyrrolo[2,3-d]pyrimidin-2-amine |
| 751 | (R)-methyl(methylimino)(6-((4bR,8aR)-4b,7,7-trimethyl-2-((4-(1-methylpiperidin-4- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)-16-sulfanone |
| 752 | (S)-methyl(methylimino)(6-((4bR,8aR)-4b,7,7-trimethyl-2-((4-(1-methylpiperidin-4- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)-16-sulfanone |
| 753 | dimethyl(6-((4bR,8aR)-4b,7,7-trimethyl-2-((4-(4-methylpiperazin-1-yl)phenyl)amino)- |
| | 4b,7,8,8a-tetrahydropyrano[3′,4′:4,5]pyrrolo[2,3-d]pyrimidin-9(5H)-yl)pyridin-2-yl)phosphine |
| | oxide |
| 754 | 1-((6-((4bR,8aR)-4b,7,7-trimethyl-2-((4-(1-methylazetidin-3-yl)phenyl)amino)-4b,7,8,8a- |
| | tetrahydropyrano[3′,4′:4,5]pyrrolo[2,3-d]pyrimidin-9(5H)-yl)pyridin-2-yl)imino)tetrahydro-1H- |
| | 116-thiophene 1-oxide |
| 755, 766 | (4bR,8aR)-4b,7,7-trimethyl-N-(4-(4-methylpiperazin-1-yl)phenyl)-9-(6-((R)-2,2,2-trifluoro-1- |
| | (methylamino)ethyl)pyridin-2-yl)-4b,5,7,8,8a,9-hexahydropyrano[3′,4′:4,5]pyrrolo[2,3- |
| | d]pyrimidin-2-amine and (4bR,8aR)-4b,7,7-trimethyl-N-(4-(4-methylpiperazin-1-yl)phenyl)-9- |
| | (6-((S)-2,2,2-trifluoro-1-(methylamino)ethyl)pyridin-2-yl)-4b,5,7,8,8a,9- |
| | hexahydropyrano[3′,4′:4,5]pyrrolo[2,3-d]pyrimidin-2-amine |
| 757 | (4bR,8aR)-4b,7,7-trimethyl-N-(4-(1-methylpiperidin-4-yl)phenyl)-9-(6-(methylsulfonyl)pyridin- |
| | 2-yl)-4b,5,7,8,8a,9-hexahydropyrano[3′,4′:4,5]pyrrolo[2,3-d]pyrimidin-2-amine |
| 758 | (4bR,8aR)-9-(6-methoxypyridin-2-yl)-4b,7,7-trimethyl-N-(4-(4-methylpiperazin-1-yl)phenyl)- |
| | 4b,5,7,8,8a,9-hexahydropyrano[3′,4′:4,5]pyrrolo[2,3-d]pyrimidin-2-amine |
| 759 | (4bR,8aR)-4b,7,7-trimethyl-N-(4-(4-methylpiperazin-1-yl)phenyl)-9-(6-methylpyridin-2-yl)- |
| | 4b,5,7,8,8a,9-hexahydropyrano[3′,4′:4,5]pyrrolo[2,3-d]pyrimidin-2-amine |
| 760, 761 | (1R,2R)-2-phenyl-N-((4bR,8aR)-4b,7,7-trimethyl-9-(6-((methylamino)methyl)pyridin-2-yl)- |
| | 4b,5,7,8,8a,9-hexahydropyrano[3′,4′:4,5]pyrrolo[2,3-d]pyrimidin-2-yl)cyclopropane-1- |
| | carboxamide and (1S,2S)-2-phenyl-N-((4bR,8aR)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-yl)cyclopropane-1-carboxamide |
| 762 | N-((4bR,8aR)-4b,7,7-trimethyl-9-(6-((methylamino)methyl)pyridin-2-yl)-4b,5,7,8,8a,9- |
| | hexahydropyrano[3′,4′:4,5]pyrrolo[2,3-d]pyrimidin-2-yl)cyclopropanecarboxamide |
| 763, 764 | (1S,2S)-2-(1-methyl-1H-pyrazol-4-yl)-N-((4bR,8aR)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-yl)cyclopropane-1-carboxamide and (1R,2R)-2-(1-methyl-1H-pyrazol-4-yl)-N- |
| | ((4bR,8aR)-4b,7,7-trimethyl-9-(6-((methylamino)methyl)pyridin-2-yl)-4b,5,7,8,8a,9- |
| | hexahydropyrano[3′,4′:4,5]pyrrolo[2,3-d]pyrimidin-2-yl)cyclopropane-1-carboxamide |
| 765 | (S)-methyl(methylimino)(6-((4bR,8aR)-4b,7,7-trimethyl-2-((4-(4-methylpiperazin-1- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)-16-sulfanone |
| 766 | (R)-methyl(methylimino)(6-((4bR,8aR)-4b,7,7-trimethyl-2-((4-(4-methylpiperazin-1- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)-16-sulfanone |
| 767 | (4bR,8aR)-N-(4-(2H-1,2,3-triazol-2-yl)phenyl)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-amine |
| 768, 769 | (1R,2R)-2-(pyridin-3-yl)-N-((4bR,8aR)-4b,7,7-trimethyl-9-(6-((methylamino)methyl)pyridin-2- |
| | yl)-4b,5,7,8,8a,9-hexahydropyrano[3′,4′:4,5]pyrrolo[2,3-d]pyrimidin-2-yl)cyclopropane-1- |
| | carboxamide and (1S,2S)-2-(pyridin-3-yl)-N-((4bR,8aR)-4b,7,7-trimethyl-9-(6- |
| | ((methylamino)methyl)pyridin-2-yl)-4b,5,7,8,8a,9-hexahydropyrano[3′,4′:4,5]pyrrolo[2,3- |
| | d]pyrimidin-2-yl)cyclopropane-1-carboxamide |
| 770, 771 | (R)-methyl(methylimino)(6-((4bR,8aR)-4b,7,7-trimethyl-2-((4-(6-methyl-2,6- |
| | diazaspiro[3.3]heptan-2-yl)phenyl)amino)-4b,7,8,8a-tetrahydropyrano[3′,4′:4,5]pyrrolo[2,3- |
| | d]pyrimidin-9(5H)-yl)pyridin-2-yl)-16-sulfanone and (S)-methyl(methylimino)(6-((4bR,8aR)- |
| | 4b,7,7-trimethyl-2-((4-(6-methyl-2,6-diazaspiro[3.3]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)-16-sulfanone |
| 772 | tetrahydro-2H-pyran-4-yl((4bR,8aR)-9-(6-((dimethyl(oxo)-16-sulfaneylidene)amino)pyridin-2- |
| | yl)-4b,7,7-trimethyl-4b,5,7,8,8a,9-hexahydropyrano[3′,4′:4,5]pyrrolo[2,3-d]pyrimidin-2- |
| | yl)carbamate |
| 773 | (S)-1-methylpyrrolidin-3-yl((4bR,8aR)-9-(6-((dimethyl(oxo)-16-sulfaneylidene)amino)pyridin- |
| | 2-yl)-4b,7,7-trimethyl-4b,5,7,8,8a,9-hexahydropyrano[3′,4′:4,5]pyrrolo[2,3-d]pyrimidin-2- |
| | yl)carbamate |
| 774 | (4bR,8aR)-9-(6-cyclopropylpyridin-2-yl)-4b,7,7-trimethyl-N-(4-(4-methylpiperazin-1- |
| | yl)phenyl)-4b,5,7,8,8a,9-hexahydropyrano[3′,4′:4,5]pyrrolo[2,3-d]pyrimidin-2-amine |
| 775 | dimethyl(6-((4bR,8aR)-4b,7,7-trimethyl-2-((4-(6-methyl-2,6-diazaspiro[3.3]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)phosphine oxide |
| 776 | (S)-5-methyl-1-(6-((4bR,8aR)-4b-methyl-2-((4-(6-methyl-2,6-diazaspiro[3.3]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)pyrrolidin-2-one |
| 777, 778 | dimethyl(6-((4bR,8aR)-4b,7,7-trimethyl-2-((4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)phosphine oxide and |
| | dimethyl(6-((4bR,8aR)-4b,7,7-trimethyl-2-((4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)phosphine oxide |
| 779 | 4-(((4bR,8aR)-9-(6-(dimethylphosphoryl)pyridin-2-yl)-4b,7,7-trimethyl-4b,5,7,8,8a,9- |
| | hexahydropyrano[3′,4′:4,5]pyrrolo[2,3-d]pyrimidin-2-yl)amino)-1-methylpyridin-2(1H)-one |
| 780 | 4-(((4bR,8aR)-9-(6-(dimethylphosphoryl)pyridin-2-yl)-4b,7,7-trimethyl-4b,5,7,8,8a,9- |
| | hexahydropyrano[3′,4′:4,5]pyrrolo[2,3-d]pyrimidin-2-yl)amino)-1-(tetrahydro-2H-pyran-4- |
| | yl)pyridin-2(1H)-one |
| 781 | (S)-4-methyl-3-(6-((4bR,8aR)-4b,7,7-trimethyl-2-((2-oxo-1-(tetrahydro-2H-pyran-4-yl)-1,2- |
| | dihydropyridin-4-yl)amino)-4b,7,8,8a-tetrahydropyrano[3′,4′:4,5]pyrrolo[2,3-d]pyrimidin-9(5H)- |
| | yl)pyridin-2-yl)oxazolidin-2-one |
| 782, 783 | 4-(((4bR,8aR)-9-(6-(dimethylphosphoryl)pyridin-2-yl)-4b,7,7-trimethyl-4b,5,7,8,8a,9- |
| | hexahydropyrano[3′,4′:4,5]pyrrolo[2,3-d]pyrimidin-2-yl)amino)-1-((S)-tetrahydrofuran-3- |
| | yl)pyridin-2(1H)-one and 4-(((4bR,8aR)-9-(6-(dimethylphosphoryl)pyridin-2-yl)-4b,7,7- |
| | trimethyl-4b,5,7,8,8a,9-hexahydropyrano[3′,4′:4,5]pyrrolo[2,3-d]pyrimidin-2-yl)amino)-1-((R)- |
| | tetrahydrofuran-3-yl)pyridin-2(1H)-one |
| 784 | (S)-4-methyl-3-(6-((4bR,8aR)-4b-methyl-2-((4-(6-methyl-2,6-diazaspiro[3.3]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one |
| 785 | 4,4-dimethyl-3-(6-((4bR,8aR)-4b,7,7-trimethyl-2-((4-(6-methyl-2,6-diazaspiro[3.3]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one |
| 786 | 5,5-dimethyl-1-(6-((4bR,8aR)-4b,7,7-trimethyl-2-((4-(6-methyl-2,6-diazaspiro[3.3]heptan-2- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)pyrrolidin-2-one |
| 787, 788 | (R)-7-ethyl-2-((4bR,8aR)-4b,7,7-trimethyl-2-((4-(4-methylpiperazin-1-yl)phenyl)amino)- |
| | 4b,7,8,8a-tetrahydropyrano[3′,4′:4,5]pyrrolo[2,3-d]pyrimidin-9(5H)-yl)-6,7-dihydro-5H- |
| | cyclopenta[b]pyridin-7-ol and (S)-7-ethyl-2-((4bR,8aR)-46,7,7-trimethyl-2-((4-(4- |
| | methylpiperazin-1-yl)phenyl)amino)-4b,7,8,8a-tetrahydropyrano[3′,4′:4,5]pyrrolo[2,3- |
| | d]pyrimidin-9(5H)-yl)-6,7-dihydro-5H-cyclopenta[b]pyridin-7-ol |
| 789, 790 | (S)-7-ethyl-2-((4bR,8aR)-4b,7,7-trimethyl-2-((4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)-6,7-dihydro-5H- |
| | cyclopenta[b]pyridin-7-ol and (R)-7-ethyl-2-((4bR,8aR)-4b,7,7-trimethyl-2-((4-((1R,4R)-5- |
| | methyl-2,5-diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)-6,7-dihydro-5H- |
| | cyclopenta[b]pyridin-7-ol |
| 791, 792 | (S)-7-ethyl-2-((4bR,8aR)-4b,7,7-trimethyl-2-((4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)-6,7-dihydro-5H- |
| | cyclopenta[b]pyridin-7-ol and (R)-7-ethyl-2-((4bR,8aR)-4b,7,7-trimethyl-2-((4-((1S,4S)-5- |
| | methyl-2,5-diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)-6,7-dihydro-5H- |
| | cyclopenta[b]pyridin-7-ol |
| 793, 794 | (S)-4-methyl-3-(6-((4bR,8aR)-4b-methyl-2-((4-((R)-1-methylpyrrolidin-3-yl)phenyl)amino)- |
| | 4b,7,8,8a-tetrahydropyrano[3′,4′:4,5]pyrrolo[2,3-d]pyrimidin-9(5H)-yl)pyridin-2-yl)oxazolidin-2- |
| | one and (S)-4-methyl-3-(6-((4bR,8aR)-4b-methyl-2-((4-((S)-1-methylpyrrolidin-3- |
| | yl)phenyl)amino)-4b,7,8,8a-tetrahydropyrano[3′,4′:4,5]pyrrolo[2,3-d]pyrimidin-9(5H)-yl)pyridin- |
| | 2-yl)oxazolidin-2-one |
| 795, 796 | 4,4-dimethyl-3-(6-((4bR,8aR)-4b,7,7-trimethyl-2-((4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one and |
| | 4,4-dimethyl-3-(6-((4bR,8aR)-4b,7,7-trimethyl-2-((4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)oxazolidin-2-one |
| 797, 798 | 5,5-dimethyl-1-(6-((4bR,8aR)-4b,7,7-trimethyl-2-((4-((1R,4R)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one and |
| | 5,5-dimethyl-1-(6-((4bR,8aR)-4b,7,7-trimethyl-2-((4-((1S,4S)-5-methyl-2,5- |
| | diazabicyclo[2.2.1]heptan-2-yl)phenyl)amino)-4b,7,8,8a- |
| | tetrahydropyrano[3′,4′:4,5]pyrrolo[2,3-d]pyrimidin-9(5H)-yl)pyridin-2-yl)pyrrolidin-2-one |
| 799 | dimethyl((6-((4bR,8aR)-4b,7,7-trimethyl-2-(pyridazin-4-ylamino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 800,801 | (4bR,8aR)-4b,7,7-trimethyl-N-(4-((1S,4S)-5-methyl-2,5-diazabicyclo[2.2.1]heptan-2- |
| | yl)phenyl)-9-(pyridin-2-yl)-4b,5,7,8,8a,9-hexahydropyrano[3′,4′:4,5]pyrrolo[2,3-d]pyrimidin-2- |
| | amine and (4bR,8aR)-4b,7,7-trimethyl-N-(4-((1R,4R)-5-methyl-2,5-diazabicyclo[2.2.1]heptan- |
| | 2-yl)phenyl)-9-(pyridin-2-yl)-4b,5,7,8,8a,9-hexahydropyrano[3′,4′:4,5]pyrrolo[2,3-d]pyrimidin- |
| | 2-amine |
| 802 | 1-cyclobutyl-4-(((4bR,8aR)-9-(6-((dimethyl(oxo)-16-sulfaneylidene)amino)pyridin-2-yl)-4b,7,7- |
| | trimethyl-4b,5,7,8,8a,9-hexahydropyrano[3′,4′:4,5]pyrrolo[2,3-d]pyrimidin-2-yl)amino)pyridin- |
| | 2(1H)-one |
| 803 | (4bR,8aR)-4b,7,7-trimethyl-N-(4-((3aR,6aS)-5-methylhexahydropyrrolo[3,4-c]pyrrol-2(1H)- |
| | yl)phenyl)-9-(pyridin-2-yl)-4b,5,7,8,8a,9-hexahydropyrano[3′,4′:4,5]pyrrolo[2,3-d]pyrimidin-2- |
| | amine |
| 804, 805 | 4-(((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)amino)-1-((R)-1- |
| | methylpyrrolidin-3-yl)pyridin-2(1H)-one and 4-(((4bR,8aR)-9-(6-((dimethyl(oxo)-16- |
| | sulfaneylidene)amino)pyridin-2-yl)-4b,7,7-trimethyl-4b,5,7,8,8a,9- |
| | hexahydropyrano[3′,4′:4,5]pyrrolo[2,3-d]pyrimidin-2-yl)amino)-1-((S)-1-methylpyrrolidin-3- |
| | yl)pyridin-2(1H)-one |
| 806 | 1-(1-methylpiperidin-4-yl)-4-(((4bR,8aR)-4b,7,7-trimethyl-9-(pyridin-2-yl)-4b,5,7,8,8a,9- |
| | hexahydropyrano[3′,4′:4,5]pyrrolo[2,3-d]pyrimidin-2-yl)amino)pyridin-2(1H)-one |
| 807 | (4bR,8aR)-9-(6-(difluoromethyl)pyridin-2-yl)-4b,7,7-trimethyl-N-(4-(4-methylpiperazin-1- |
| | yl)phenyl)-4b,5,7,8,8a,9-hexahydropyrano[3′,4′:4,5]pyrrolo[2,3-d]pyrimidin-2-amine |
| 808, 809 | (4bR,8aR)-N-(4-((5R)-1,4-diazabicyclo[3.2.1]octan-4-yl)phenyl)-4b,7,7-trimethyl-9-(pyridin-2- |
| | yl)-4b,5,7,8,8a,9-hexahydropyrano[3′,4′:4,5]pyrrolo[2,3-d]pyrimidin-2-amine and (4bR,8aR)- |
| | N-(4-((5S)-1,4-diazabicyclo[3.2.1]octan-4-yl)phenyl)-4b,7,7-trimethyl-9-(pyridin-2-yl)- |
| | 4b,5,7,8,8a,9-hexahydropyrano[3′,4′:4,5]pyrrolo[2,3-d]pyrimidin-2-amine |
| 810 | 4-(((4bR,8aR)-9-(6-(2-hydroxypropan-2-yl)pyridin-2-yl)-4b,7,7-trimethyl-4b,5,7,8,8a,9- |
| | hexahydropyrano[3′,4′:4,5]pyrrolo[2,3-d]pyrimidin-2-yl)amino)-1-(1-methylpiperidin-4- |
| | yl)pyridin-2(1H)-one |
| 811,812 | 4-(((4bR,8aR)-9-((S)-7-ethyl-7-hydroxy-6,7-dihydro-5H-cyclopenta[b]pyridin-2-yl)-4b,7,7- |
| | trimethyl-4b,5,7,8,8a,9-hexahydropyrano[3′,4′:4,5]pyrrolo[2,3-d]pyrimidin-2-yl)amino)-1-(1- |
| | methylpiperidin-4-yl)pyridin-2(1H)-one and 4-(((4bR,8aR)-9-((R)-7-ethyl-7-hydroxy-6,7- |
| | dihydro-5H-cyclopenta[b]pyridin-2-yl)-4b,7,7-trimethyl-4b,5,7,8,8a,9- |
| | hexahydropyrano[3′,4′:4,5]pyrrolo[2,3-d]pyrimidin-2-yl)amino)-1-(1-methylpiperidin-4- |
| | yl)pyridin-2(1H)-one |
| 813, 814 | (4bR,8aR)-9-(6-(difluoromethyl)pyridin-2-yl)-4b,7,7-trimethyl-N-(4-((1S,4S)-5-methyl-2,5- |
| | diazabicyclo[2.2.1]heptan-2-yl)phenyl)-4b,5,7,8,8a,9-hexahydropyrano[3′,4′:4,5]pyrrolo[2,3- |
| | d]pyrimidin-2-amine and (4bR,8aR)-9-(6-(difluoromethyl)pyridin-2-yl)-4b,7,7-trimethyl-N-(4- |
| | ((1R,4R)-5-methyl-2,5-diazabicyclo[2.2.1]heptan-2-yl)phenyl)-4b,5,7,8,8a,9- |
| | hexahydropyrano[3′,4′:4,5]pyrrolo[2,3-d]pyrimidin-2-amine |
| 815 | ((6-((4bR,8aR)-2-((5-(difluoromethyl)pyridin-3-yl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 816 | 4-(((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)amino)-1,6- |
| | dimethylpyridin-2(1H)-one |
| 817 | dimethyl((6-((4bR,8aR)-4b,7,7-trimethyl-2-(pyrimidin-5-ylamino)-4b,7,8,8a- |
| | tetrahydropyrano[3′,4′:4,5]pyrrolo[2,3-d]pyrimidin-9(5H)-yl)pyridin-2-yl)imino)-16-sulfanone |
| 818 | 5-(((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)amino)nicotinonitrile |
| 819 | (4bR,8aR)-N-(4-(4-(dimethylamino)piperidin-1-yl)phenyl)-4b,7,7-trimethyl-9-(pyridin-2-yl)- |
| | 4b,5,7,8,8a,9-hexahydropyrano[3′,4′:4,5]pyrrolo[2,3-d]pyrimidin-2-amine |
| 820, 821 | (4bR,8aR)-N-(4-((S)-3-(dimethylamino)pyrrolidin-1-yl)phenyl)-4b,7,7-trimethyl-9-(pyridin-2-yl)- |
| | 4b,5,7,8,8a,9-hexahydropyrano[3′,4′:4,5]pyrrolo[2,3-d]pyrimidin-2-amine and (4bR,8aR)-N-(4- |
| | ((R)-3-(dimethylamino)pyrrolidin-1-yl)phenyl)-4b,7,7-trimethyl-9-(pyridin-2-yl)-4b,5,7,8,8a,9- |
| | hexahydropyrano[3′,4′:4,5]pyrrolo[2,3-d]pyrimidin-2-amine |
| 822 | (4bR,8aR)-N-(4-(4-(dimethylamino)-4-methylpiperidin-1-yl)phenyl)-4b,7,7-trimethyl-9-(pyridin- |
| | 2-yl)-4b,5,7,8,8a,9-hexahydropyrano[3′,4′:4,5]pyrrolo[2,3-d]pyrimidin-2-amine |
| 823 | (4bR,8aR)-4b,7,7-trimethyl-N-(4-(1-methyl-1,8-diazaspiro[4.5]decan-8-yl)phenyl)-9-(pyridin-2- |
| | yl)-4b,5,7,8,8a,9-hexahydropyrano[3′,4′:4,5]pyrrolo[2,3-d]pyrimidin-2-amine |
| 824 | 4-(((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)amino)-1-(1-(oxetan-3- |
| | yl)piperidin-4-yl)pyridin-2(1H)-one |
| 825 | 4-(((4bR,8aR)-9-(6-((dimethyl(oxo)-16-sulfaneylidene)amino)pyridin-2-yl)-4b,7,7-trimethyl- |
| | 4b,5,7,8,8a,9-hexahydropyrano[3′,4′:4,5]pyrrolo[2,3-d]pyrimidin-2-yl)amino)-1-(1-(2- |
| | methoxyethyl)piperidin-4-yl)pyridin-2(1H)-one |
| 826 | 2-methyl-6-((4bR,8aR)-4b,7,7-trimethyl-2-((4-(4-methylpiperazin-1-yl)phenyl)amino)- |
| | 4b,7,8,8a-tetrahydropyrano[3′,4′:4,5]pyrrolo[2,3-d]pyrimidin-9(5H)-yl)pyridazin-3(2H)-one |
| 827 | (4bR,8aR)-4b,7,7-trimethyl-N-(4-(4-methylpiperazin-1-yl)phenyl)-9-(pyrazin-2-yl)- |
| | 4b,5,7,8,8a,9-hexahydropyrano[3′,4′:4,5]pyrrolo[2,3-d]pyrimidin-2-amine |
| 828 | (4bR,8aR)-4b,7,7-trimethyl-N-(4-(4-methylpiperazin-1-yl)phenyl)-9-(pyridazin-3-yl)- |
| | 4b,5,7,8,8a,9-hexahydropyrano[3′,4′:4,5]pyrrolo[2,3-d]pyrimidin-2-amine |
| 829 | 2-isopropyl-6-((4bR,8aR)-4b,7,7-trimethyl-2-((4-(4-methylpiperazin-1-yl)phenyl)amino)- |
| | 4b,7,8,8a-tetrahydropyrano[3′,4′:4,5]pyrrolo[2,3-d]pyrimidin-9(5H)-yl)pyridazin-3(2H)-one |
| 830 | ((6-((4bR,8aR)-2-((1-ethyl-1H-pyrazol-3-yl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |
| 831 | ((6-((4bR,8aR)-2-((1-cyclopropyl-1H-pyrazol-3-yl)amino)-4b,7,7-trimethyl-4b,7,8,8a- |
| | tetrahydropyrano [3′,4′:4,5]pyrrolo[2,3-d]pyrimidin-9(5H)-yl)pyridin-2-yl)imino)dimethyl-16- |
| | sulfanone |
| 832 | ((6-((4bR,8aR)-2-((1-isopropyl-1H-pyrazol-3-yl)amino)-4b,7,7-trimethyl-4b,7,8,8a- |
| | tetrahydropyrano[3′,4′:4,5]pyrrolo[2,3-d]pyrimidin-9(5H)-yl)pyridin-2-yl)imino)dimethyl-16- |
| | sulfanone |

### Methods of Treatment

Also provided herein are methods of treating cancer in a subject with a compound of Formula (I), or a pharmaceutical salt thereof, or a pharmaceutical composition thereof. Replication stress is present in many cancers, and as noted herein, it can in some cases be exacerbated by one or more factors, such as genetic features of the cancer and/or administration of DNA-damaging agents, DNA repair inhibiting agents, and/or radiation.

Accordingly, provided herein is a method of treating a cancer in a subject in need thereof, the method including identifying the cancer as having replication stress; and administering to the subject an effective amount of a compound of Formula (I), or a pharmaceutical salt thereof, or a pharmaceutical composition thereof.

Identifying the cancer as having replication stress can include any appropriate method of identification, such as the methods described herein. For example, in some embodiments, identifying the cancer as having replication stress includes staining for replication forks in a sample from the subject. In some embodiments, identifying the cancer as having replication stress includes detecting a biomarker of replication stress in a sample from the subject. A biomarker of replication stress can include any appropriate biomarker or set of biomarkers. In some embodiments, a biomarker of replication stress includes Ki-67, Cyclin E, POLD3, γH2AX, FANCD2, or a combination thereof. In some embodiments, a biomarker of replication stress includes pH2AX Ser139, pATR Thr1989, pCHK1 Ser345, pRPA32 Ser33, or a combination thereof. In some embodiments, a biomarker of replication stress includes an activated oncogene. In some embodiments, a biomarker of replication stress includes an inactivated tumor suppressor gene.

Also provided herein is a method of treating a cancer in a subject in need thereof, the method comprising administering an effective amount of a compound of Formula (I), or a pharmaceutical salt thereof, or a pharmaceutical composition thereof to a subject identified as having a cancer having replication stress.

In some cases, a genetic characteristic of a cancer can be indicative that the cancer can be treated effectively with a compound of Formula (I), or a pharmaceutical salt thereof, or a pharmaceutical composition thereof. Some such genetic characteristics include one or more inactivated tumor suppressor genes and/or one or more activated oncogenes.

Accordingly, also provided herein is a method of treating a cancer in a subject in need thereof, the method including: identifying the cancer as having an inactivated tumor suppressor gene; and administering to the subject an effective amount of a compound of Formula (I), or a pharmaceutical salt thereof, or a pharmaceutical composition thereof. Also provided herein is a method of treating a cancer in a subject in need thereof, the method including administering an effective amount of a compound of Formula (I), or a pharmaceutical salt thereof, or a pharmaceutical composition thereof to a subject identified as having a cancer having an inactivated tumor suppressor gene.

An inactivation of a tumor suppressor gene can be achieved by any appropriate mechanism, such as those described herein. In some embodiments, an inactivated tumor suppressor gene includes an inactivation selected from the group consisting of a deletion of the gene, an inactivating mutation in the protein product of the gene, an inactivating translocation in the protein product of the gene, a transcriptional silencing of the gene, an epigenetic alteration of the gene, degradation of mRNA products of the gene, degradation of protein products of the gene, and combinations thereof.

An inactive tumor suppressor gene can be any appropriate inactivated tumor suppressor gene, such as any of those described herein. In some embodiments, the tumor suppressor gene is selected from the group consisting of p53, RB1, CDKN2A, BRCA1, BRCA2, FBXW7, SETD2, NOTCH1, and a combination thereof.

In some embodiments, the inactivated tumor suppressor gene includes a mutation in the protein product of a p53 gene. In some embodiments, the inactivated tumor suppressor gene includes a deleted p53 gene. In some embodiments, the inactivated tumor suppressor gene includes a mutation in the protein product of a CDKN2A gene. In some embodiments, the inactivated tumor suppressor gene includes a mutation in the protein product of a NOTCH1 gene. In some embodiments, the inactivated tumor suppressor gene includes a deleted FBXW7 gene. In some embodiments, the inactivated tumor suppressor gene includes a mutation in the protein product of a FBXW7 gene. In some embodiments, the inactivated tumor suppressor gene includes a mutation in the protein product of a RB1 gene. In some embodiments, the inactivated tumor suppressor gene includes a deleted BRCA1 gene. In some embodiments, the inactivated tumor suppressor gene includes a mutation in the protein product of a BRCA1 gene. In some embodiments, the inactivated tumor suppressor gene includes a BRCA1 gene with a hypermethylated promoter region. In some embodiments, the inactivated tumor suppressor gene includes a deleted BRCA2 gene. In some embodiments, the inactivated tumor suppressor gene includes a mutation in the protein product of a BRCA2 gene. In some embodiments, the inactivated tumor suppressor gene includes a BRCA2 gene with a hypermethylated promoter region. In some embodiments, the inactivated tumor suppressor gene includes a mutation in the protein product of a NOTCH1 gene. In some embodiments, the inactivated tumor suppressor gene includes a mutation in the protein product of a SETD2 gene.

In some embodiments, the inactivated tumor suppressor gene is selected from the group consisting of a mutation in the protein product of a p53 gene, a deleted p53 gene, a mutation in the protein product of a CDKN2A gene, a mutation in the protein product of a NOTCH1 gene, a deleted FBXW7 gene, a mutation in the protein product of a FBXW7 gene, a mutation in the protein product of a RB1 gene, a deleted BRCA1 gene, a mutation in the protein product of a BRCA1 gene, a BRCA1 gene with a hypermethylated promoter region, a deleted BRCA2 gene, a mutation in the protein product of a BRCA2 gene, a BRCA2 gene with a hypermethylated promoter region, a mutation in the protein product of a NOTCH1 gene, a mutation in the protein product of a SETD2 gene, and a combination thereof.

Also provided herein is a method of treating a cancer in a subject in need thereof, the method including: identifying the cancer as having an activated oncogene; and administering to the subject an effective amount of a compound of Formula (I), or a pharmaceutical salt thereof, or a pharmaceutical composition thereof. Also provided herein is a method of treating a cancer in a subject in need thereof, the method including administering an effective amount of a compound of Formula (I), or a pharmaceutical salt thereof, or a pharmaceutical composition thereof to a subject identified as having a cancer having an activated oncogene.

An activation of an oncogene can be achieved by any appropriate mechanism, such as those described herein For example, in some embodiments, the activated oncogene has an activation selected from the group consisting of an amplification of the oncogene, an activating mutation of the protein product of the oncogene, an activating translocation of the protein product of the oncogene, transcriptional activation of the oncogene, epigenetic alteration of the oncogene, overexpression of the protein product of the oncogene, and combinations thereof.

An activated oncogene can be any appropriate oncogene, such as those described herein. In some embodiments, the oncogene is selected from the group consisting of cyclin E, CDC25A, Myc, a RAS gene, and combinations thereof. In some embodiments, RAS gene includes a KRAS gene. In some embodiments, the RAS gene includes an NRAS gene. In some embodiments, the RAS gene includes an HRAS gene.

In some embodiments, the activated oncogene includes an amplified cyclin E gene. In some embodiments, the activated oncogene includes an overexpression of the protein product of the CDC25A gene. In some embodiments, the activated oncogene includes an amplified Myc gene. In some embodiments, the activated oncogene includes an activating translocation in the protein product of a Myc gene. In some embodiments, the activated oncogene includes a transcriptionally activated Myc gene. In some embodiments, the activated oncogene includes a mutation in the protein product of a RAS gene. In some embodiments, the mutated RAS gene includes a mutation at position G12 of the protein product of the RAS gene. In some embodiments, the mutated RAS gene includes a mutation at position G13 of the protein product of the RAS gene. In some embodiments, wherein the mutated RAS gene includes a mutation at position Q61 of the protein product of the RAS gene. In some embodiments, the RAS gene includes a KRAS gene.

In some embodiments, the activated oncogene is selected from the group consisting of an amplified cyclin E gene, an overexpression of the protein product of the CDC25A gene, an amplified Myc gene, an activating translocation in the protein product of a Myc gene, a transcriptionally activated Myc gene, a mutation in the protein product of a RAS gene, and a combination thereof. In some embodiments, the mutated RAS gene includes a mutation at position G12, G13, Q61, or a combination thereof, of the protein product of the RAS gene. In some embodiments, the RAS gene includes a KRAS gene.

In some embodiments, the compounds of the present disclosure are particularly useful wherein the cancer is selected from one or more of uterine, ovarian, breast, gastric, colorectal, and non-small cell lung.

In the field of medical oncology, it is normal practice to use a combination of different forms of treatment to treat each subject with cancer. In medical oncology the other component(s) of such conjoint treatment or therapy in addition to compositions provided herein may be, for example, surgery, radiotherapy, and chemotherapeutic agents, such as other kinase inhibitors, kinase inhibitors, signal transduction inhibitors, and/or monoclonal antibodies. For example, a surgery may be open surgery or minimally invasive surgery. Compounds of Formula (I), or a pharmaceutically acceptable salt thereof therefore may also be useful as adjuvants to cancer treatment, that is, they can be used in combination with one or more additional therapies or therapeutic agents, for example, a chemotherapeutic agent that works by the same or by a different mechanism of action. In some embodiments, a compound of Formula (I), or a pharmaceutically acceptable salt thereof, can be used prior to administration of an additional therapeutic agent or additional therapy. For example, a subject in need thereof can be administered one or more doses of a compound of Formula (I), or a pharmaceutically acceptable salt thereof for a period of time and then undergo at least partial resection of the tumor. In some embodiments, the treatment with one or more doses of a compound of Formula (I), or a pharmaceutically acceptable salt thereof reduces the size of the tumor (e.g., the tumor burden) prior to the at least partial resection of the tumor. In some embodiments, a subject in need thereof can be administered one or more doses of a compound of Formula (I), or a pharmaceutically acceptable salt thereof for a period of time and under one or more rounds of radiation therapy. In some embodiments, the treatment with one or more doses of a compound of Formula (I), or a pharmaceutically acceptable salt thereof reduces the size of the tumor (e.g., the tumor burden) prior to the one or more rounds of radiation therapy.

In some embodiments of any of the methods described herein, the compound of Formula (I), or a pharmaceutically acceptable salt thereof, is administered in combination with an effective amount of at least one additional therapeutic agent selected from one or more additional therapies or therapeutic (e.g., chemotherapeutic or immunomodulatory) agents.

Non-limiting examples of additional therapeutic agents include: PARP inhibitors, other DNA repair inhibiting agents (e.g. topoisomerase inhibitors, DNA-dependent protein kinase (DNA-PK) inhibitors, ATM inhibitors, Aurora kinase inhibitors (such as Aurora A and/or Aurora B inhibitors), ATR inhibitors, and CHK1 inhibitors), signal transduction pathway inhibitors, Bcr-Abl inhibitors, histone deacetylase (HDAC) inhibitors, checkpoint inhibitors, modulators of the apoptosis pathway, cytotoxic chemotherapeutics, angiogenesis-targeted therapies, immune-targeted agents, including immunotherapy, and radiotherapy. In some embodiments, the additional therapeutic agent is an immunotherapy.

Non-limiting examples of checkpoint inhibitors include ipilimumab, tremelimumab, nivolumab, pidilizumab, MPDL3208A, MEDI4736, MSB0010718C, BMS-936559, BMS-956559, BMS-935559 (MDX-1105), AMP-224, and pembrolizumab.

In some embodiments, cytotoxic chemotherapeutics are selected from bleomycin, cabazitaxel, capecitabine, carboplatin, cisplatin, cyclophosphamide, cytarabine, dacarbazine, daunorubicin, docetaxel, paclitaxel, doxorubicin, etoposide, fluorouracil, gemcitabine, irinotecan, lomustine, methotrexate, mitomycin C, oxaliplatin, paclitaxel, pemetrexed, temozolomide, and vincristine.

Non-limiting examples of angiogenesis-targeted therapies include aflibercept and bevacizumab.

In some embodiments, a DNA repair inhibiting agent can include a topoisomerase I inhibitor, a topoisomerase II inhibitor, a PARP inhibitor, an ATR inhibitor, a Chk inhibitor, a DNA-dependent protein kinase (DNA-PK) inhibitor, an ATM inhibitor, an Aurora kinase inhibitor (such as an Aurora A and/or B inhibitor), or a combination thereof.

Non-limiting examples of PARP inhibitors include olaparib, niraparib, rucaparib, talazoparib, and veliparib.

Non-limiting examples of ATR inhibitors include AZD6738, BAY1895344, and M6620.

Non-limiting examples of Chk1 inhibitors include prexasertib, GDC-0575, SCH 900776, and SRA737.

Non-limiting examples of DNA-PK inhibitors include AZD7648, M3814, LY294002, nedisertib, and samotolisib.

Non-limiting examples of ATM inhibitors include KU55933, AZD0156, AZD1390, dactosilib, and berzosertib.

Non-limiting examples of Aurora kinase inhibitors include LY3295668, ZM447439, tozasertib, hesparadin, alisertib, and MLN8054.

Non-limiting examples of modulators of the apoptosis pathway include Bcl-2 inhibitors such as obataclax, venetoclax, and navitoclax.

In some embodiments, signal transduction pathway inhibitors include Ras-Raf-MEK-ERK pathway inhibitors (e.g., binimetinib, selumetinib, encorafenib, sorafenib, trametinib, and vemurafenib) or PI3K-Akt-mTOR-S6K pathway inhibitors (e.g., sirolimus, everolimus, rapamycin, perifosine, temsirolimus).

Non-limiting examples of Bcr-Abl inhibitors include imatinib, nilotinib, dasatinib, bosutinib, ponatinib, and bafetinib.

Non-limiting examples of HDAC inhibitors include pabinostat, vorinostat, belinostat, panobinostat, entinostat, tacedinaline, and mocetinostat.

Non-limiting examples of platinum-based chemotherapeutics include carboplatin, cisplatin, and oxaplatin. Non-limiting examples of alkylating agents include cyclophosphamide, carmustine, busulfan, procarbazine, dacarbazine, temozoloamide, thiotepa, and mitomycin C. Non-limiting examples of nucleobase, nucleoside, and/or nucleotide analogs include fluorouracil, cytarabine, gemcitabine, azacitidine, and decitabine. Non-limiting examples of topoisomerase I inhibitors include topotecan, irinotecan, belotecan, and camptothecin. Non-limiting examples of topoisomerase II inhibitors include etoposide, tenoposide, doxorubicin, daunorubicin, epirubicin, and idarubacin.

The term “immunotherapy” refers to an agent that modulates the immune system. In some embodiments, an immunotherapy can increase the expression and/or activity of a regulator of the immune system. In some embodiments, an immunotherapy can decrease the expression and/or activity of a regulator of the immune system. In some embodiments, an immunotherapy can recruit and/or enhance the activity of an immune cell.

In some embodiments, the immunotherapy is a cellular immunotherapy (e.g., adoptive T-cell therapy, dendritic cell therapy, natural killer cell therapy). In some embodiments, the cellular immunotherapy is sipuleucel-T (APC8015; Provenge™; Plosker (2011) Drugs 71(1): 101-108). In some embodiments, the cellular immunotherapy includes cells that express a chimeric antigen receptor (CAR). In some embodiments, the cellular immunotherapy is a CAR-T cell therapy. In some embodiments, the CAR-T cell therapy is tisagenlecleucel (Kymriah™).

In some embodiments, the immunotherapy is an antibody therapy (e.g., a monoclonal antibody, a conjugated antibody). In some embodiments, the antibody therapy is bevacizumab (Mvasti™, Avastin®), trastuzumab (Herceptin®), avelumab (Bavencio®), rituximab (MabThera™, Rituxan®), edrecolomab (Panorex), daratumuab (Darzalex®), olaratumab (Lartruvo™), ofatumumab (Arzerra®), alemtuzumab (Campath®), cetuximab (Erbitux®), oregovomab, pembrolizumab (Keytruda®), dinutiximab (Unituxin®), obinutuzumab (Gazyva®), tremelimumab (CP-675,206), ramucirumab (Cyramza®), ublituximab (TG-1101), panitumumab (Vectibix®), elotuzumab (Empliciti™), avelumab (Bavencio®), necitumumab (Portrazza™) cirmtuzumab (UC-961), ibritumomab (Zevalin®), isatuximab (SAR650984), nimotuzumab, fresolimumab (GC1008), lirilumab (INN), mogamulizumab (Poteligeo®), ficlatuzumab (AV-299), denosumab (Xgeva®), ganitumab, urelumab, pidilizumab or amatuximab.

In some embodiments, the immunotherapy is an antibody-drug conjugate. In some embodiments, the antibody-drug conjugate is gemtuzumab ozogamicin (Mylotarg™) inotuzumab ozogamicin (Besponsa®), brentuximab vedotin (Adcetris®), ado-trastuzumab emtansine (TDM-1; Kadcyla®), mirvetuximab soravtansine (IMGN853) or anetumab ravtansine

In some embodiments, the immunotherapy includes blinatumomab (AMG103; Blincyto®) or midostaurin (Rydapt).

In some embodiments, the immunotherapy includes a toxin. In some embodiments, the immunotherapy is denileukin diftitox (Ontak®).

In some embodiments, the immunotherapy is a cytokine therapy. In some embodiments, the cytokine therapy is an interleukin 2 (IL-2) therapy, an interferon alpha (IFNα) therapy, a granulocyte colony stimulating factor (G-CSF) therapy, an interleukin 12 (IL-12) therapy, an interleukin 15 (IL-15) therapy, an interleukin 7 (IL-7) therapy or an erythropoietin-alpha (EPO) therapy. In some embodiments, the IL-2 therapy is aldesleukin (Proleukin®). In some embodiments, the IFNα therapy is IntronA® (Roferon-A®). In some embodiments, the G-CSF therapy is filgrastim (Neupogen®).

In some embodiments, the immunotherapy is an immune checkpoint inhibitor. In some embodiments, the immunotherapy includes one or more immune checkpoint inhibitors. In some embodiments, the immune checkpoint inhibitor is a CTLA-4 inhibitor, a PD-1 inhibitor or a PD-L1 inhibitor. In some embodiments, the CTLA-4 inhibitor is ipilimumab (Yervoy®) or tremelimumab (CP-675,206). In some embodiments, the PD-1 inhibitor is pembrolizumab (Keytruda®) or nivolumab (Opdivo®). In some embodiments, the PD-L1 inhibitor is atezolizumab (Tecentriq®), avelumab (Bavencio®) or durvalumab (Imfinzi™).

Non-limiting examples of radiotherapy include radioiodide therapy, external-beam radiation, and radium 223 therapy.

In some embodiments, the one or more additional therapies or therapeutic agents are selected from cytarabine, fludarabine, cisplatin, carboplatin, docetaxel, gemcitabine, belinostat, radiotherapy, irinotecan, olaparib, pemetrexed, savolitinib, and temozolomide.

In some cases, a cancer having replication stress and/or including a genetic characteristic indicative that the cancer can be treated effectively with a compound of Formula (I), or a pharmaceutical salt thereof, or a pharmaceutical composition thereof can be treated with a combination of a compound of Formula (I), or a pharmaceutical salt thereof, or a pharmaceutical composition thereof and another agent that promotes genomic instability, such as a DNA-damaging agent, a DNA repair inhibiting agent, radiation, or a combination thereof.

Accordingly, in some embodiments, the methods described herein can further include administering to the subject a DNA-damaging agent, a DNA repair inhibiting agent, radiation, or a combination thereof.

In some cases, identification of replication stress might not be carried out on the cancer or might not be able to be carried out on a cancer. In some cases, genetic analysis might not be carried out on the cancer or might not be able to be carried out on a cancer. In some cases, a cancer might be negative for a genetic characteristic of a cancer can be indicative that the cancer can be treated effectively with a compound of Formula (I), or a pharmaceutical salt thereof, or a pharmaceutical composition thereof. However, many first-line treatment regimens for cancer include a DNA-damaging agent, a DNA repair inhibiting agent, or a combination thereof. In some such cases, a compound of Formula (I), or a pharmaceutical salt thereof, or a pharmaceutical composition thereof can still be indicated for treatment with a compound of Formula (I), or a pharmaceutical salt thereof, or a pharmaceutical composition thereof, as the combination of factors can promote mitotic collapse, thereby treating the cancer.

Accordingly, provided herein is a method of treating a cancer in a subject in need thereof, the method comprising: (i) administering to the subject an effective amount of a therapy comprising: (a) a DNA-damaging agent; (b) a DNA repair inhibiting agent; (c) radiation; (d) a DNA-damaging agent and a DNA repair inhibiting agent; (e) a DNA-damaging agent and radiation; (f) a DNA repair inhibiting agent and radiation; or (g) a DNA-damaging agent, a DNA repair inhibiting agent, and radiation; and (ii) after (i), administering to the subject an effective amount of a compound of Formula (I), or a pharmaceutical salt thereof, or a pharmaceutical composition thereof. Also provided herein is a method of treating a cancer in a subject in need thereof, the method comprising: administering an effective amount of a compound of Formula (I), or a pharmaceutical salt thereof, or a pharmaceutical composition thereof to a subject previously administered one or more doses of a therapy comprising: (a) a DNA-damaging agent; (b) a DNA repair inhibiting agent; (c) radiation; (d) a DNA-damaging agent and a DNA repair inhibiting agent; (e) a DNA-damaging agent and radiation; (f) a DNA repair inhibiting agent and radiation; or (g) a DNA-damaging agent, a DNA repair inhibiting agent, and radiation. In some embodiments, the therapy (e.g., of (a) to (g)) is continued to be administered to the subject as combination therapy with the compound of Formula (I), or a pharmaceutical salt thereof, or a pharmaceutical composition thereof. Also provided herein is a method of treating a cancer in a subject in need thereof, the method comprising: administering to the subject: (i) an effective amount of a compound of Formula (I), or a pharmaceutical salt thereof, or a pharmaceutical composition thereof; and (ii) an effective amount of a therapy comprising: (a) a DNA-damaging agent; (b) a DNA repair inhibiting agent; (c) radiation; (d) a DNA-damaging agent and a DNA repair inhibiting agent; (e) a DNA-damaging agent and radiation; (f) a DNA repair inhibiting agent and radiation; or (g) a DNA-damaging agent, a DNA repair inhibiting agent, and radiation.

In some embodiments, the compound of Formula (I), or a pharmaceutical salt thereof, or a pharmaceutical composition thereof and the therapy (e.g., of (a) to (g)) are administered simultaneously as separate dosages. In some embodiments, the compound of Formula (I), or a pharmaceutical salt thereof, or a pharmaceutical composition thereof and the therapy (e.g., of (a) to (g)) are administered separate dosages sequentially in any order.

A DNA-damaging agent can be any appropriate DNA-damaging agent, such as those described herein. In some embodiments, the DNA-damaging agent is selected from the group consisting of a platinum-based chemotherapy, an alkylating agent, a nucleobase, nucleoside, or nucleotide analog, and combinations thereof. In some embodiments, the platinum-based chemotherapy comprises carboplatin, cisplatin, oxaplatin, or a combination thereof. In some embodiments, the alkylating agent comprises cyclophosphamide, carmustine, busulfan, procarbazine, dacarbazine, temozoloamide, thiotepa, mitomycin C, or combinations thereof. In some embodiments, the nucleobase, nucleoside, or nucleotide analog comprises fluorouracil, cytarabine, gemcitabine, azacitidine, decitabine, or combinations thereof.

A DNA repair inhibiting agent can be any appropriate DNA repair inhibiting agent, such as those described herein. In some embodiments, the DNA repair inhibiting agent is selected from the group consisting of a topoisomerase I inhibitor, a topoisomerase II inhibitor, a PARP inhibitor, an ATR inhibitor, a Chk inhibitor, a DNA-dependent protein kinase (DNA-PK) inhibitor, an ATM inhibitors, an Aurora kinase inhibitor (such as Aurora A and/or Aurora B inhibitors), and a combination thereof.

In some embodiments, the topoisomerase I inhibitor comprises topotecan, irinotecan, belotecan, camptothecin, or combinations thereof. In some embodiments, the topoisomerase II inhibitor comprises etoposide, tenoposide, doxorubicin, daunorubicin, epirubicin, idarubacin, or combinations thereof. In some embodiments, the PARP inhibitor comprises olaparib, niraparib, rucaparib, talazoparib, veliparib, or combinations thereof. In some embodiments, the ATR inhibitor comprises AZD6738, BAY1895344, M6620, or a combination thereof. In some embodiments, the Chk1 inhibitor comprises prexasertib, GDC-0575, SCH 900776, SRA737, or a combination thereof. In some embodiments, the DNA-PK inhibitor comprises AZD7648, M3814, LY294002, nedisertib, samotolisib, or combinations thereof. In some embodiments, the ATM inhibitor comprises KU55933, AZD0156, AZD1390, dactosilib, berzosertib, or combinations thereof. In some embodiments, the Aurora kinase inhibitor comprises LY3295668, ZM447439, tozasertib, hesparadin, alisertib, MLN8054, or combinations thereof.

In some embodiments, the topoisomerase I inhibitor is topotecan, irinotecan, belotecan, camptothecin, or a combination thereof. In some embodiments, the topoisomerase II inhibitor is etoposide, tenoposide, doxorubicin, daunorubicin, epirubicin, idarubacin, or a combination thereof. In some embodiments, the PARP inhibitor is olaparib, niraparib, rucaparib, talazoparib, veliparib, or a combination thereof. In some embodiments, the ATR inhibitor is AZD6738, BAY1895344, M6620, or a combination thereof. In some embodiments, the Chk1 inhibitor is prexasertib, GDC-0575, SCH 900776, SRA737, or a combination thereof. In some embodiments, the DNA-PK inhibitor is AZD7648, M3814, LY294002, nedisertib, samotolisib, or a combination thereof. In some embodiments, the ATM inhibitor is KU55933, AZD0156, AZD1390, dactosilib, berzosertib, or a combination thereof. In some embodiments, the Aurora kinase inhibitor is LY3295668, ZM447439, tozasertib, hesparadin, alisertib, MLN8054, or a combination thereof.

Also provided herein is a method for treating a subject diagnosed with or identified as having a cancer associated with replication stress, e.g., any of the exemplary cancers disclosed herein, comprising administering to the subject an effective amount of a compound of Formula (I), or a pharmaceutically acceptable salt thereof, or a pharmaceutical composition thereof as defined herein.

Also provided herein is a method for treating cancer in a subject in need thereof, the method comprising: determining that the cancer is associated with replication stress; and administering to the subject an effective amount of a compound of Formula (I), or a pharmaceutical salt thereof, or a pharmaceutical composition thereof. Also provided herein is a method for treating cancer in a subject in need thereof, the method comprising: administering an effective amount of a compound of Formula (I), or a pharmaceutical salt thereof, or a pharmaceutical composition thereof to a subject identified as having a cancer associated with replication stress.

In some embodiments of any of the methods described herein, the method further includes administering an additional therapy or therapeutic agent to the subject. An additional therapy or therapeutic agent can be any appropriate therapy or therapeutic agent. In some embodiments, the additional therapy or therapeutic agent is selected from radiotherapy, cytotoxic chemotherapeutics, kinase-targeted therapeutics, kinase-targeted therapeutics, apoptosis modulators, signal transduction inhibitors, immune-targeted therapies, angiogenesis-targeted therapies, and combinations thereof. In some embodiments, the additional therapy or therapeutic agent is selected from kinase-targeted therapeutics, kinase-targeted therapeutics, apoptosis modulators, signal transduction inhibitors, immune-targeted therapies, angiogenesis-targeted therapies, and combinations thereof. In some embodiments, the additional therapy or therapeutic agent is an immune-targeted therapy. In some embodiments, the immune-targeted therapy is an immunotherapy.

Also provided herein is a method for inhibiting mammalian cell proliferation, comprising contacting the mammalian cell with a compound of Formula (I), or a pharmaceutical salt thereof. Also provided herein is a method for inducing mitotic collapse in a mammalian cell, comprising contacting the mammalian cell with a compound of Formula (I), or a pharmaceutical salt thereof. In some embodiments, the contacting occurs in vivo. In some embodiments, the contacting occurs in vitro. A mammalian cell can be any appropriate species or type of cell. In some embodiments, the mammalian cell is a mammalian immune cell. In some embodiments, the mammalian cell is a mammalian cancer cell. In some embodiments, the mammalian cancer cell is a mammalian cancer cell having replicative stress. In some embodiments, the mammalian cancer cell has an inactivated tumor suppressor gene. In some embodiments, the mammalian cancer cell has an activated oncogene. In some embodiments, the method further includes contacting the mammalian cell with a DNA-damaging agent, a DNA repair inhibitor, radiation, or a combination thereof.

Also provided herein is use of a compound of Formula (I), or a pharmaceutical salt thereof in the manufacture of a medicament for the treatment of cancer. In some embodiments, the cancer is a cancer having replication stress. In some embodiments, the cancer is a cancer having an inactivated tumor suppressor gene. In some embodiments, the cancer is a cancer having an activated oncogene. In some embodiments, the medicament is labeled for concurrent use with a DNA-damaging agent, a DNA repair inhibitor, radiation therapy, or a combination thereof. In some embodiments, the medicament is labeled for use subsequent to a DNA-damaging agent, a DNA repair inhibitor, radiation therapy, or a combination thereof.

In some embodiments of any of the methods or uses described herein, the cancer is a hematological cancer. In some embodiments of any of the methods or uses described herein, the cancer is a solid tumor. In some embodiments of any of the methods or uses described herein, the cancer is small cell lung cancer, ovarian cancer, solid tumors with BRCA mutations, head and neck cancer squamous cell carcinoma, adenocarcinoma of the pancreas, acute myeloid leukemia, osteosarcoma, multiple myeloma, epithelial ovarian cancers, triple negative breast cancer, cervical cancer, mantle cell lymphoma and diffuse large B-cell lymphoma, laryngeal squamous cell carcinoma, basal-like breast cancer, medulloblastoma, oropharyngeal cancers, sarcoma, kidney cancer, clear cell renal cell carcinoma, acute lymphoblastic leukemia, pediatric gliomas, head and neck precancer, Ewing sarcoma, gastrointestinal stromal tumors, giant cell tumor of bone, clear cell ovarian cancer, mucinous ovarian cancer, primary peritoneal carcinoma, serous surface papillary carcinoma, teratoma, dysgerminoma, endodermal sinus tumors, choriocarcinomas, granulosa cell tumors, granulosa-theca tumors, sertoli-leydig cell tumors, endometrial adenocarcinoma, adenosquamous carcinoma, papillary serous carcinoma, and uterine sarcoma.

In some embodiments, the subject is a human.

In some embodiments of any of the methods described herein, a compound of Formula (I) is selected from Examples 1-832 or a pharmaceutically acceptable salt thereof.

Also provided is a method for inhibiting Wee1 kinase activity in a mammalian cell, comprising contacting the mammalian cell with a compound of Formula (I). In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof to a subject having a mammalian cell having Wee1 kinase activity. In some embodiments, the mammalian cell is a mammalian immune cell. In some embodiments, the mammalian cell is a mammalian cancer cell. In some embodiments, the mammalian cancer cell is any cancer as described herein. In some embodiments, the mammalian cancer cell is a mammalian cancer cell having replication stress.

Also provided is a method for inhibiting Wee1 kinase activity in a mammalian cell, comprising contacting the mammalian cell with a compound of Formula (I). In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof to a mammal having a mammalian cell having Wee1 kinase activity. In some embodiments, the mammalian cell is a mammalian immune cell. In some embodiments, the mammalian cell is a mammalian cancer cell. In some embodiments, the mammalian cancer cell is any cancer as described herein. In some embodiments, the mammalian cancer cell is a mammalian cancer cell with replication stress. In some embodiments, the mammalian cell is a gastrointestinal mammalian cell. In some embodiments, the mammalian cell is a hematological mammalian cell.

As used herein, the term “contacting” refers to the bringing together of indicated moieties in an in vitro system or an in vivo system. For example, “contacting” a Wee1 kinase with a compound provided herein includes the administration of a compound provided herein to a subject, such as a human, having a Wee1 kinase, as well as, for example, introducing a compound provided herein into a sample containing a mammalian cellular or purified preparation containing the Wee1 kinase.

Also provided herein is a method of inhibiting mammalian cell proliferation, in vitro or in vivo, the method comprising contacting a mammalian cell with an effective amount of a compound of Formula (I), or a pharmaceutically acceptable salt thereof, or a pharmaceutical composition thereof as defined herein.

A “Wee1 kinase inhibitor” as defined herein includes any compound exhibiting Wee1 inhibition activity. In some embodiments, a Wee1 kinase inhibitor is selective for a Wee1 kinase. Exemplary Wee1 kinase inhibitors can exhibit inhibition activity (IC₅₀) against a Wee1 kinase of less than about 1000 nM, less than about 500 nM, less than about 200 nM, less than about 100 nM, less than about 50 nM, less than about 25 nM, less than about 10 nM, or less than about 1 nM as measured in an assay as described herein. In some embodiments, a Wee1 kinase inhibitor can exhibit inhibition activity (IC₅₀) against a Wee1 kinase of less than about 25 nM, less than about 10 nM, less than about 5 nM, or less than about 1 nM as measured in an assay as provided herein.

The phrase “effective amount” means an amount of compound that, when administered to a subject in need thereof, is sufficient to (i) treat a cancer (such as cancer associated with replication stress as described herein), (ii) attenuate, ameliorate, or eliminate one or more symptoms of the particular cancer, or (iii) delay the onset of one or more symptoms of the particular cancer described herein. The amount of a compound of Formula (I), or a pharmaceutically acceptable salt thereof that will correspond to such an amount will vary depending upon factors such as the particular compound, disease condition and its severity, the identity (e.g., weight) of the subject in need of treatment, but can nevertheless be routinely determined by one skilled in the art.

When employed as pharmaceuticals, compounds of Formula (I), including pharmaceutically acceptable salts thereof, can be administered in the form of pharmaceutical compositions. These compositions can be prepared in a manner well known in the pharmaceutical art, and can be administered by a variety of routes, depending upon whether local or systemic treatment is desired and upon the area to be treated. Administration can be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Oral administration can include a dosage form formulated for once-daily or twice-daily (BID) administration. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or can be, for example, by a continuous perfusion pump. Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like may be necessary or desirable.

Also provided herein are pharmaceutical compositions which contain, as the active ingredient, a compound of Formula (I) or pharmaceutically acceptable salt thereof, in combination with one or more pharmaceutically acceptable excipients. For example, a pharmaceutical composition prepared using a compound of Formula (I) or a pharmaceutically acceptable salt thereof. In some embodiments, the composition is suitable for topical administration. In making the compositions provided herein, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders. In some embodiments, the composition is formulated for oral administration. In some embodiments, the composition is a solid oral formulation. In some embodiments, the composition is formulated as a tablet or capsule.

Further provided herein are pharmaceutical compositions containing a compound of Formula (I) or a pharmaceutically acceptable salt thereof with a pharmaceutically acceptable carrier. Pharmaceutical compositions containing a compound of Formula (I) or a pharmaceutically acceptable salt thereof as the active ingredient can be prepared by intimately mixing the compound of Formula (I), or a pharmaceutically acceptable salt thereof with a pharmaceutical carrier according to conventional pharmaceutical compounding techniques. The carrier can take a wide variety of forms depending upon the desired route of administration (e.g., oral, parenteral). In some embodiments, the composition is a solid oral composition.

Suitable pharmaceutically acceptable carriers are well known in the art. Descriptions of some of these pharmaceutically acceptable carriers can be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

In preparing the compositions in oral dosage form, any of the usual pharmaceutical media can be employed. Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Solid oral preparations can also be coated with substances such as sugars or be enteric-coated so as to modulate major site of absorption. For parenteral administration, the carrier will usually consist of sterile water and other ingredients can be added to increase solubility or preservation. Injectable suspensions or solutions can also be prepared utilizing aqueous carriers along with appropriate additives. The pharmaceutical compositions herein will contain, per dosage unit, e.g., tablet, capsule, powder, injection, teaspoonful and the like, an amount of the active ingredient necessary to deliver an effective dose as described herein.

The compositions comprising a compound of Formula (I) or a pharmaceutically acceptable salt thereof can be formulated in a unit dosage form, each dosage containing from about 5 to about 1,000 mg (1 g), more usually about 100 mg to about 500 mg, of the active ingredient. The term “unit dosage form” refers to physically discrete units suitable as unitary dosages for human subjects and other subjects, each unit containing a predetermined quantity of active material (i.e., a compound of Formula (I) or a pharmaceutically acceptable salt thereof) calculated to produce the desired therapeutic effect, in association with a suitable pharmaceutical excipient.

In some embodiments, the compositions provided herein contain from about 5 mg to about 50 mg of the active ingredient. One having ordinary skill in the art will appreciate that this embodies compounds or compositions containing about 5 mg to about 10 mg, about 10 mg to about 15 mg, about 15 mg to about 20 mg, about 20 mg to about 25 mg, about 25 mg to about 30 mg, about 30 mg to about 35 mg, about 35 mg to about 40 mg, about 40 mg to about 45 mg, or about 45 mg to about 50 mg of the active ingredient.

In some embodiments, the compositions provided herein contain from about 50 mg to about 500 mg of the active ingredient. One having ordinary skill in the art will appreciate that this embodies compounds or compositions containing about 50 mg to about 100 mg, about 100 mg to about 150 mg, about 150 mg to about 200 mg, about 200 mg to about 250 mg, about 250 mg to about 300 mg, about 350 mg to about 400 mg, or about 450 mg to about 500 mg of the active ingredient. In some embodiments, the compositions provided herein contain about 10 mg, about 20 mg, about 80 mg, or about 160 mg of the active ingredient.

In some embodiments, the compositions provided herein contain from about 500 mg to about 1,000 mg of the active ingredient. One having ordinary skill in the art will appreciate that this embodies compounds or compositions containing about 500 mg to about 550 mg, about 550 mg to about 600 mg, about 600 mg to about 650 mg, about 650 mg to about 700 mg, about 700 mg to about 750 mg, about 750 mg to about 800 mg, about 800 mg to about 850 mg, about 850 mg to about 900 mg, about 900 mg to about 950 mg, or about 950 mg to about 1,000 mg of the active ingredient.

The daily dosage of the compound of Formula (I) or a pharmaceutically acceptable salt thereof can be varied over a wide range from 1.0 to 10,000 mg per adult human per day, or higher, or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 160, 200, 250 and 500 milligrams of the active ingredient for the symptomatic adjustment of the dosage to the subject to be treated. An effective amount of the drug is ordinarily supplied at a dosage level of from about 0.1 mg/kg to about 1000 mg/kg of body weight per day, or any range therein. Preferably, the range is from about 0.5 to about 500 mg/kg of body weight per day, or any range therein. More preferably, from about 1.0 to about 250 mg/kg of body weight per day, or any range therein. More preferably, from about 0.1 to about 100 mg/kg of body weight per day, or any range therein. In an example, the range can be from about 0.1 to about 50.0 mg/kg of body weight per day, or any amount or range therein. In another example, the range can be from about 0.1 to about 15.0 mg/kg of body weight per day, or any range therein. In yet another example, the range can be from about 0.5 to about 7.5 mg/kg of body weight per day, or any amount to range therein. Pharmaceutical compositions containing a compound of Formula (I) or a pharmaceutically acceptable salt thereof can be administered on a regimen of 1 to 4 times per day or in a single daily dose.

The active compound may be effective over a wide dosage range and is generally administered in a pharmaceutically effective amount. Optimal dosages to be administered can be readily determined by those skilled in the art. It will be understood, therefore, that the amount of the compound actually administered will usually be determined by a physician, and will vary according to the relevant circumstances, including the mode of administration, the actual compound administered, the strength of the preparation, the condition to be treated, and the advancement of the disease condition. In addition, factors associated with the particular subject being treated, including subject response, age, weight, diet, time of administration and severity of the subject's symptoms, will result in the need to adjust dosages.

In some embodiments, the compounds provided herein can be administered in an amount ranging from about 1 mg/kg to about 100 mg/kg. In some embodiments, the compound provided herein can be administered in an amount of about 1 mg/kg to about 20 mg/kg, about 5 mg/kg to about 50 mg/kg, about 10 mg/kg to about 40 mg/kg, about 15 mg/kg to about 45 mg/kg, about 20 mg/kg to about 60 mg/kg, or about 40 mg/kg to about 70 mg/kg. For example, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg.

One skilled in the art will recognize that both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy subjects and/or those suffering from a given disorder, can be completed according to methods well known in the clinical and medical arts.

Provided herein are pharmaceutical kits useful, for example, in the treatment of cancer (such as replication sensitive cancers), which include one or more containers containing a pharmaceutical composition comprising an effective amount of a compound provided herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### Preparation of Compounds

As disclosed herein, many of the starting materials used are commercially available or can be prepared using the routes described below using techniques known to those skilled in the art.

### General Schemes

Compounds of Formula (I) can be prepared as described in the Schemes below.

Tricyclic dihydro-pyrrolo pyrimidines of type id used as starting material in Scheme 2 were prepared via the route depicted in Scheme 1:

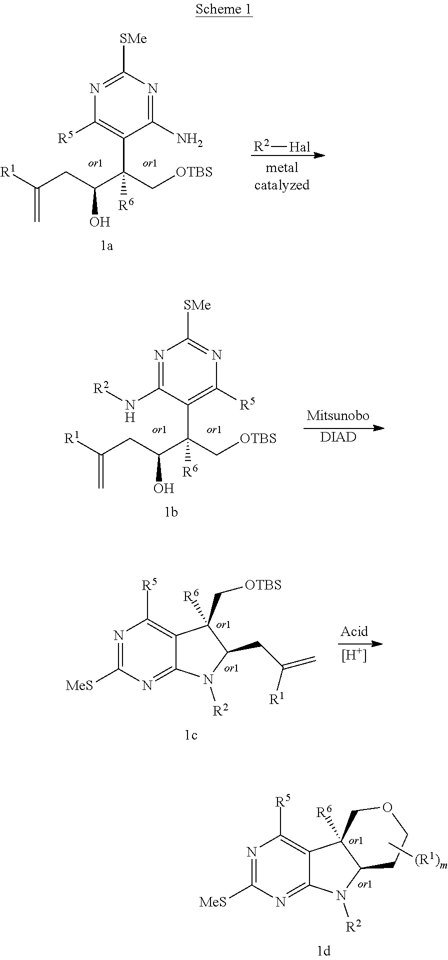

### General Procedure for Scheme 1

Hydroxy amino-pyrimidines of type 1b were prepared from functionalized aminopyrimidines of type 1a via metal-catalyzed coupling reaction with a suitable R²-substituted heteroaryl halide in the presence of an appropriate metal catalyst (such as CuI, XantPhos-Pd-G₂ and the like), an appropriate ligand (such as DMEDA, XantPhos and the like), and an appropriate base (such as Cs₂CO₃ and the like).

Intramolecular Mitsunobu reaction of the respective hydroxy amino-pyrimidines of type 1b to provide bicyclic dihydro-pyrrolopyrimidines of type 1c could be accomplished in the presence of an appropriate azodicarboxylate (such as DIAD and the like) and a suitable phosphine (such as PBu₃, PPh₃ and the like) in a suitable organic solvent (such as THF and the like).

Tricyclic dihydro-pyrrolopyrimidines of type id were prepared from bicyclic dihydro-pyrrolopyrimidines of type 1c by reaction with a suitable acid (where [H⁺] represents acids such as hydrochloric acid and the like) in a suitable organic solvent (such as 1,4-dioxane and the like).

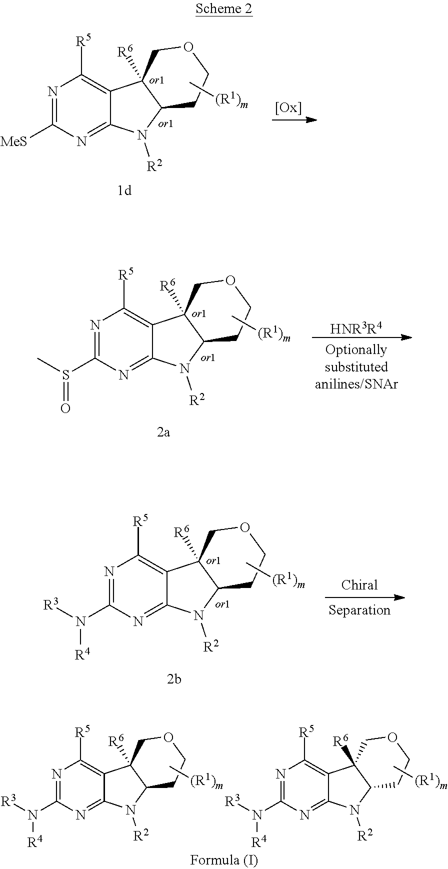

### General Procedure for Scheme 2

Pyrimidine sulfoxides of type 2a were prepared from tricyclic dihydro-pyrrolopyrimidines of type id by reaction with suitable oxidative regents (where [Ox] represents m-CPBA and the like) in a suitable organic solvent (such as toluene and the like).

Tricyclic compounds 2b representative of a compounds Formula (I) were prepared from the respective pyrimidine sulfoxides of type 2a through reaction with an optionally substituted anilines HNR³R⁴ and an appropriate base (such as DIPEA and the like) in a suitable organic solvent (such as toluene and the like).

Examples representative of Formula (I) were prepared upon the chiral separation with an appropriate condition (such as column, mobile phases, gradient condition and the like).

Alternatively, compounds of Formula (I) can be prepared in enantiomerically pure fashion as described in the Scheme 3 below.

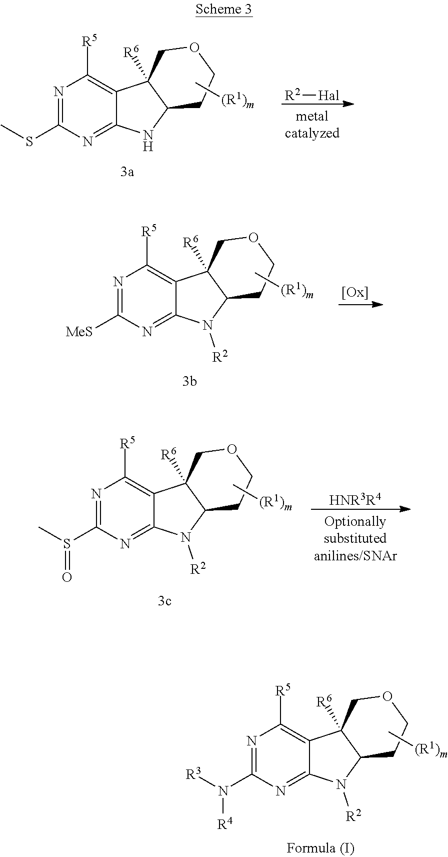

### General Procedure for Scheme 3

Tricyclic dihydro-pyrrolopyrimidines of type 3b were prepared from functionalized intermediates of type 3a via metal-catalyzed coupling reaction with a suitable R²-substituted heteroaryl halide in the presence of an appropriate metal catalyst (such as Pd₂dba₃, CuI, XantPhos-Pd-G₂ and the like), an appropriate ligand (such as BINAP, DMEDA, XantPhos and the like), and an appropriate base (such as Cs₂CO₃ and the like).

Pyrimidine sulfoxides of type 3c were prepared from tricyclic dihydro-pyrrolopyrimidines of type 3b by reaction with suitable oxidative regents (where [Ox] represents m-CPBA and the like) in a suitable organic solvent (such as toluene and the like).

Examples representative of Formula (I) were prepared from the respective pyrimidine sulfoxides of type 3c through reaction with an optionally substituted anilines HNR³R⁴ and an appropriate base (such as DIPEA and the like) in a suitable organic solvent (such as toluene and the like).

Compounds of Formula (I-D) can also be prepared as described in the Scheme 4 below.

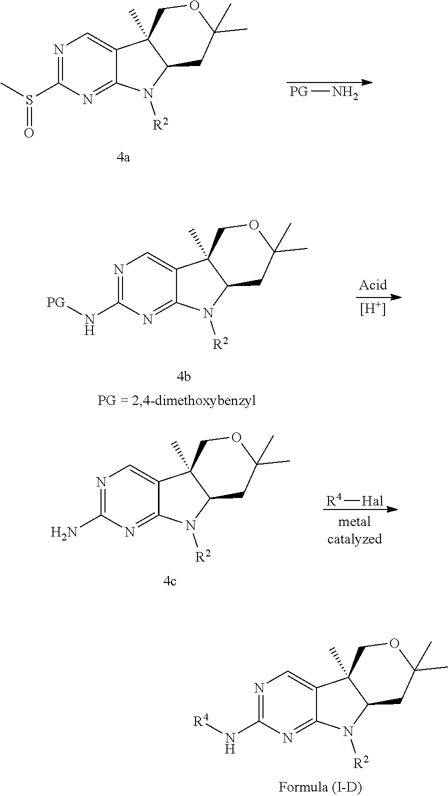

### General Procedure for Scheme 4

Amino-pyrimidines of type 4b were prepared from the respective pyrimidine sulfoxides of type 4a (representative of sulfoxides of type 3c) through reaction with a substituted amines PG-NH₂ (where PG-NH₂ represents 2,4-dimethoxylbenzylamine and the like) and an appropriate base (such as DIPEA and the like) in a suitable organic solvent (such as toluene and the like).

Compounds of type 4c were prepared from the respective amino-pyrimidines of type 4b through reaction with a suitable acid (such as TFA and the like). Examples representative of Formula (I-D) were prepared from compounds of 4c via metal-catalyzed coupling reaction with an optionally substituted heteroaryl halide R⁴-Hal in the presence of an appropriate metal catalyst (such as Xphos-Pd-G₃, XantPhos-Pd-G₃ and the like), an appropriate ligand (such as Xphos, XantPhos and the like), and an appropriate base (such as Cs₂CO₃ and the like).

### EXAMPLES

### Materials and Methods

The compounds provided herein, including salts thereof, can be prepared using known organic synthesis techniques and can be synthesized according to any of numerous possible synthetic routes.

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in Protecting Group Chemistry, 1^{st} Ed., Oxford University Press, 2000; March's Advanced Organic Chemistry: Reactions, Mechanisms, and Structure, 5^{th} Ed., Wiley-Interscience Publication, 2001; and Peturssion, S. et al., “Protecting Groups in Carbohydrate Chemistry,” J. Chem. Educ., 74(11), 1297 (1997).

Reactions sensitive to moisture or air were performed under nitrogen or argon using anhydrous solvents and reagents. The progress of reactions was determined by either analytical thin layer chromatography (TLC) usually performed with Sanpont precoated TLC plates, silica gel GF-254, layer thickness 0.25 mm or liquid chromatography-mass spectrometry (LC-MS).

Typically, the analytical LC-MS system used consisted of Shimadzu LCMS-2020 with electrospray ionization in positive ion detection mode with 20ADXR pump, SIL-20ACXR autosampler, CTO-20AC column oven, M20A PDA Detector and LCMS 2020 MS detector. The column was usually HALO a C18 30*5.0 mm, 2.7 μm. The mobile phase A is water containing 0.05% TFA and mobile phase B is acetonitrile containing 0.05% TFA. The gradient is from 5% mobile phase B to 95% in 2.0 min, hold 0.7 min, then reverting to 5% mobile phase B over 0.05 min and maintained for 0.25 min. The Column Oven (CTO-20AC) was operated at a temperature of 40.0° C. The flow rate was 1.5 mL/min, and the injection volume was 1 μl. PDA (SPD-M20A) detection was in the range 190-400 nm. The MS detector, which was configured with electrospray ionization as ionizable source; Acquisition mode: Scan; Nebulizing Gas Flow:1.5 L/min; Drying Gas Flow:15 L/min; Detector Voltage: Tuning Voltage±0.2 kv; DL Temperature: 250° C.; Heat Block Temperature: 250° C.; Scan Range: 90.00-900.00 m/z. ELSD (Alltech 3300) detector Parameters: Drift Tube Temperature:60±5° C.; N2 Flow-Rate: 1.8±0.2 L/min. Mobile phase gradients were optimized for the individual compounds.

The GC-MS system was usually performed with Shimadzu GCMS-QP2010 Ultra with FID and MS Detector. The MS detector of acquisition mode: Start Time: 2.00 min; End Time: 9.00 min; ACQ Mode: Scan; Event Time: 0.30 sec; Scan Speed: 2000; Start m/z: 50.00; End m/z: 550.00; Ion Source temperature: 200.00° C.; Interface temperature: 250.00° C.; Solvent Cut Time: 2.00 min.

Preparative HPLC purifications were usually performed with Waters Auto purification system (2545-2767) with a 2489 UV detector. The column was Waters C18, 19×150 mm, 5 μm; XBridge Prep OBD C18 Column, 30×150 mm 5 μm; XSelect CSH Prep C18 OBD Column, 5 μm, 19×150 mm; XBridge Shield RP18 OBD Column, 30×150 mm, 5 μm; Xselect CSH Fluoro Phenyl, 30×150 mm, 5 μm; YMC-Actus Triart C18, 30×150 mm, 5 μm. The mobile phases consisted of mixtures of acetonitrile (5-95%) in water containing 0.1% FA or 10 mmol/L NH₄HCO₃. Flow rates were maintained at 25 mL/min, the injection volume was 1200 μL, and the UV detector used two channels 254 nm and 220 nm. Mobile phase gradients were optimized for the individual compounds.

Chiral analytical chromatography was performed on one of Chiralpak AS, AD, Chiralcel OD, OJ Chiralpak IA, IB, IC, ID, IE, IF, IG, IH columns (Daicel Chemical Industries, Ltd.) (R,R)-Whelk-O1, (S,S)-Whelk-O1 columns (Regis technologies, Inc.) CHIRAL Cellulose-SB, SC, SA columns (YMC Co., Ltd.) at different column size (50×4.6 mm, 100×4.6 mm, 150×4.6 mm, 250×4.6 mm, 50×3.0 mm, 100×3.0 mm) with noted percentage of either ethanol in hexane (% Et/Hex) or isopropanol in hexane (% IPA/Hex) as isocratic solvent systems, or by supercritical fluid (SFC) conditions. Chiral preparative chromatography was conducted on one of Chiralpak AS, AD, Chiralcel OD, OJ Chiralpak IA, IB, IC, ID, IE, IF, IG, IH columns (Daicel Chemical Industries, Ltd.) (R,R)-Whelk-O1, (S,S)-Whelk-O1 columns (Regis technologies, Inc.) CHIRAL Cellulose-SB, SC, SA columns (YMC Co., Ltd.) at different column size (250×20 mm, 250×30 mm, 250×50 mm) with desired isocratic solvent systems identified on chiral analytical chromatography or by supercritical fluid (SFC) conditions.

Concentration of solutions was carried out on a rotary evaporator under reduced pressure. Flash column chromatography was usually performed using a Biotage Flash Chromatography apparatus (Dyax Corp.) on silica gel (40-60 μM, 60 Å pore size) in pre-packed cartridges of the size noted. ¹H NMR spectra were acquired at 400 MHz spectrometers (or 300 MHz spectrometers) in DMSO-d₆ solutions unless otherwise noted. Chemical shifts were reported in parts per million (ppm). Tetramethylsilane (TMS) was used as internal reference in DMSO-d₆ solutions, and residual CH₃OH peak or TMS was used as internal reference in CD₃OD solutions. Coupling constants (J) were reported in hertz (Hz).

TABLE 3
Abbreviations.

| | |
|---|---|
| AcOH/HOAc | Acetic acid |
| BINAP | 2,2′-bis(diphenylphosphino)-1,1′-binaphthyl |
| Boc₂O | Di-tert-butyl dicarbonate |
| Brettphos | 2-(Dicyclohexylphosphino)3,6-dimethoxy-2′,4′,6′-triisopropyl-1,1′-biphenyl |
| n-BuLi | n-Butyllithium |
| CH₃CN/ACN | Acetonitrile |
| Cu(OAc)₂ | Copper(II) acetate |
| CS₂CO₃ | Cesium carbonate |
| DAST | Diethylaminosulfur trifluoride |
| DCM | Dichloromethane or methylene chloride |
| DIAD | Diisopropyl azodicarboxylate |
| DIBAL-H | Diisobutylaluminium hydride |
| DIPEA/DIEA | N-ethyl-N-isopropyl-propan-2-amine |
| DMEDA | N,N′-Dimethylethylenediamine |
| DMF | N,N-Dimethylformamide |
| DMSO | Dimethyl sulfoxide |
| DMP | Dess-Martin periodinane |
| dtbbpy | 4,4′-Di-tert-butyl-2,2′-dipyridyl |
| EA, EtOAc | Ethyl acetate |
| FA | Formic acid |
| h | hour(s) |
| HCHO | Formaldehyde |
| H₂O | Water |
| Imd | Imidazole |
| (Ir[dF(CF3)ppy]2(dtbpy)) | [4,4′-Bis(1,1-dimethylethyl)-2,2′-bipyridine-N1, N1′]bis[3,5-difluoro-2-[5- |
| PF6 | (trifluoromethyl)-2-pyridinyl-N]phenyl-C]Iridium(III) hexafluorophosphate |
| [Ir(dF(Me)ppy)2(dtbbpy)] | Iridium(III) bis[2-(2,4-difluorophenyl)-5-methylpyridine-N,C20]-4,40-di-tert- |
| PF6 | butyl-2,20-bipyridine hexafluorophosphate |
| LDA | Lithium diisopropylamide |
| LIHMDS | Lithium Hexamethyldisilazide |
| m-CPBA | meta-Chloroperoxybenzoic acid |
| Mel | lodomethane |
| min | Minutes |
| mmol | Millimole |
| mol | Mole |
| NaBH₃CN | Sodium cyanoborohydride |
| NaHCO₃ | Sodium bicarbonate |
| NaN₃ | Sodium azide |
| NaHMDS | Sodium bis(trimethylsilyl)amide |
| NH₃ | Ammonia |
| NH₄HCO₃ | Ammonium bicarbonate |
| NH₃•H₂O | Ammonium hydroxide |
| NH₂SO₃H | Sulfamic acid |
| NaClO₂ | Sodium chlorite |
| Na₂CO₃ | Sodium carbonate |
| NaH | Sodium hydride |
| NaOH | Sodium hydroxide |
| Na₂SO₄ | Sodium sulfate |
| NiBr₂(DME) | Nickel(II) bromide ethylene glycol dimethyl ether complex |
| NiCl₂(DME) | Nickel(II) chloride ethylene glycol dimethyl ether complex |
| NMM | N-methyl morpholine |
| PBu₃ | Tributylphosphine |
| PE | Petroleum ether |
| Pd(dppf)Cl₂•DCM | [1,1′-Bis(diphenylphosphino)ferrocene]dichloropalladium(II) |
| | dichloromethane |
| Pd₂dba₃ | Tris(dibenzylideneacetone)dipalladium |
| PTSA | p-toluenesulfonic acid |
| r.t. | room temperature |
| Rt | retention time |
| SEMCl | 2-(Trimethylsilyl)ethoxymethyl chloride |
| SOCl₂ | Thionyl chloride |
| TBAF | Tetrabutylammonium fluoride |
| TBSOTf | Tert-butyldimethylsilyl trifluoromethanesulfonate |
| TEA | Triethylamine |
| TEACl | Tetraethylammonium Chloride |
| TFA | Trifluoroacetic acid |
| THF | Tetrahydrofuran |
| TMEDA | N,N,N′,N′-Tetramethylethylenediamine |
| Tol | Toluene |
| Xantphos | 4,5-Bis(diphenylphosphino)-9,9-dimethylxanthene |

### Preparation of Intermediates

### Intermediate 1: rel-(2S,3S)-2-(4-amino-2-(methylthio)pyrimidin-5-yl)-1-((tert-butyldimethylsilyl)oxy)-2,5-dimethylhex-5-en-3-ol

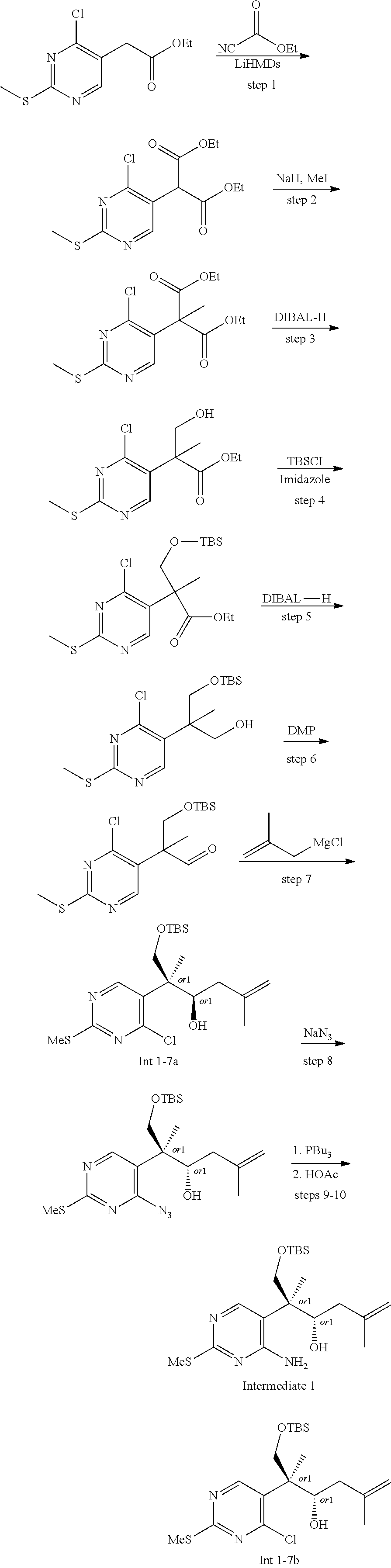

### Step 1. diethyl 2-(4-chloro-2-methylsulfanyl-pyrimidin-5-yl)propanedioate

To a solution of ethyl 2-(4-chloro-2-methylsulfanyl-pyrimidin-5-yl)acetate (10.00 g, 40.53 mmol) in THF (100 mL) was added LiHMDS (2 M in THF, 20.3 mL, 2 eq.) at −78° C. under N₂ atmosphere. The mixture was allowed to stir at −78° C. for 1 h. Then ethyl carbonocyanidate (8.05 g, 81.06 mmol, 2 eq.) was added slowly. The reaction mixture was warmed to room temperature and stirred for 3 h. The reaction was monitored by LC-MS. Ice/water (300 mL) was added to the reaction. The resulting mixture was extracted with EA (3×150 mL). The combined organic layers were washed with brine (2×100 mL), dried over anhydrous Na₂SO₄, filtered and concentrated under reduced pressure. The residue was purified by silica gel column chromatography (eluting with 1:3 EA/PE) to afford diethyl 2-(4-chloro-2-methylsulfanyl-pyrimidin-5-yl)propanedioate (12.00 g, 37.64 mmol, 93% yield) as a yellow oil. LCMS (ES, m/z): 319, 321 [M+H]⁺, Rt 0.769 min.

### Step 2. diethyl 2-(4-chloro-2-methylsulfanyl-pyrimidin-5-yl)-2-methyl-propane dioate

To a solution of diethyl 2-(4-chloro-2-methylsulfanyl-pyrimidin-5-yl)propanedioate (11.00 g, 34.51 mmol, 1 eq.) in THF (100 mL) was added NaH (2.76 g, 60% suspension in mineral oil, 69.01 mmol, 2 eq.) at 0° C. in 30 mins. The mixture was stirred for another 30 mins at 0° C. under N₂ atmosphere. Then Mel (9.80 g, 69.01 mmol, 2 eq.) was added dropwise. The mixture was stirred at 60° C. for 12 h under N₂ atmosphere. The reaction was monitored by LC-MS. The reaction mixture was allowed to cool down to room temperature and quenched by ice/water (300 mL). The aqueous layer was extracted with EA (3×100 mL). The combined organic layers were washed with brine (2×200 mL), dried over anhydrous Na₂SO₄, filtered and concentrated under reduced pressure. The residue was purified by silica gel column chromatography (eluting with 1:3 EA/PE) to afford diethyl 2-(4-chloro-2-methylsulfanyl-pyrimidin-5-yl)-2-methyl-propanedioate (10 g, 30.05 mmol, 77% yield) as a yellow oil. LCMS (ES, m/z): 333, 335 [M+H]⁺, Rt 0.914 min.

### Step 3. ethyl 2-(4-chloro-2-methylsulfanyl-pyrimidin-5-yl)-3-hydroxy-2-methyl-propanoate

To a solution of diethyl 2-(4-chloro-2-methylsulfanyl-pyrimidin-5-yl)-2-methyl-propanedioate (10.30 g, 30.95 mmol, 1 eq.) in ether (100 mL) was added DIBAL-H (1 M in toluene, 61.90 mL, 2 eq.) at −78° C. under N₂ atmosphere. The mixture was stirred at room temperature for 2 h under N₂ atmosphere. The reaction was monitored by LC-MS. The mixture was quenched by saturated potassium sodium tartrate (250 mL) and extracted with DCM (3×100 mL). The combined organic layers were washed with brine (2×100 mL), dried over anhydrous Na₂SO₄, filtered and concentrated under reduced pressure. The residue was purified by silica gel column chromatography (eluting with 1:1 EA/PE) to afford ethyl 2-(4-chloro-2-methylsulfanyl-pyrimidin-5-yl)-3-hydroxy-2-methyl-propanoate (4.8 g, 16.51 mmol, 53% yield) as a yellow oil. LCMS (ES, m/z): 291, 293 [M+H]⁺, Rt 0.655 min.

### Step 4. ethyl 3-[tert-butyl(dimethyl)silyl]oxy-2-(4-chloro-2-methylsulfanyl-pyrimidin-5-yl)-2-methyl-propanoate

To a mixture of ethyl 2-(4-chloro-2-methylsulfanyl-pyrimidin-5-yl)-3-hydroxy-2-methyl-propanoate (5.30 g, 18.22 mmol, 1 eq.) and Imd (2.48 g, 36.43 mmol, 2 eq.) in DCM (50 mL) was added TBSCl (3.31 g, 21.93 mmol, 1.2 eq.). The mixture was stirred at r.t. for 3 h. The reaction was monitored by LC-MS. Then ice/water (100 mL) was added. The resulting mixture was extracted with DCM (3×50 mL). The combined organic layers were washed with brine (2×100 mL), dried over anhydrous Na₂SO₄, filtered and concentrated under reduced pressure. The residue was purified by silica gel column chromatography (eluting with 1:3 EA/PE) to afford ethyl 3-[tert-butyl(dimethyl)silyl]oxy-2-(4-chloro-2-methylsulfanyl-pyrimidin-5-yl)-2-methyl-propanoate (6.4 g, 15.80 mmol, 96% yield) as a yellow oil. ¹H NMR (300 MHz, DMSO-d6) δ 8.62 (s, 1H), 4.26-3.90 (m, 4H), 2.53 (s, 3H), 1.56 (s, 3H), 1.14 (t, J=7.2 Hz, 3H), 0.76 (s, 9H), 0.00 (s, 3H), −0.09 (s, 3H). LCMS (ES, m/z): 405, 407 [M+H]⁺, Rt 0.907 min.

### Step 5. 3-[tert-butyl(dimethyl)silyl]oxy-2-(4-chloro-2-methylsulfanyl-pyrimidin-5-yl)-2-methyl-propan-1-ol

To a solution of ethyl 3-[tert-butyl(dimethyl)silyl]oxy-2-(4-chloro-2-methylsulfanyl-pyrimidin-5-yl)-2-methyl-propanoate (2 g, 4.95 mmol, 1 eq.) in DCM (20 mL) was added dropwise DIBAL-H (1M in DCM, 10 mL, 2 eq.) at −78° C. for 1 h under N₂ atmosphere. Then the mixture was stirred for another 1 h at room temperature. The reaction was monitored by LC-MS. The reaction was quenched with saturated sodium potassium tartrate solution (100 mL), and then the mixture was extracted with EA (3×80 mL). The combined organic layers were washed with brine (100 mL), dried over anhydrous Na₂SO₄, filtered and concentrated under vacuum. The residue was purified by flash chromatography (eluting with 1:2 EA/PE) to afford 3-[tert-butyl(dimethyl)silyl]oxy-2-(4-chloro-2-methylsulfanyl-pyrimidin-5-yl)-2-methyl-propan-1-ol (1.24 g, 3.38 mmol, 69% yield) as a colorless oil. LCMS (ES, m/z): 363, 365 [M+H]⁺, Rt 1.323 min.

### Step 6. 3-[tert-butyl(dimethyl)silyl]oxy-2-(4-chloro-2-methylsulfanyl-pyrimidin-5-yl)-2-methyl-propanal

To a solution of 3-[tert-butyl(dimethyl)silyl]oxy-2-(4-chloro-2-methylsulfanyl-pyrimidin-5-yl)-2-methyl-propan-1-ol (3.10 g, 8.54 mmol, 1 eq.) in DCM (100 mL) was added DMP (7.24 g, 17.08 mmol, 2 eq.) at 0° C. in portions. The mixture was stirred at r.t. for 4 h. The reaction was monitored by LC-MS. The mixture was washed with water (2×100 mL). The organic layer was dried over anhydrous Na₂SO₄, filtered and concentrated under reduced pressure. The residue was purified by silica gel column chromatography (eluting with 1:3 EA/PE) to afford 3-[tert-butyl(dimethyl)silyl]oxy-2-(4-chloro-2-methylsulfanyl-pyrimidin-5-yl)-2-methyl-propanal (2.80 g, 7.76 mmol, 91% yield) as a colorless oil. LCMS (ES, m/z): 361, 363 [M+H]⁺, Rt 0.924 min.

### Step 7. rel-(2S,3S)-1-((tert-butyldimethylsilyl)oxy)-2-(4-chloro-2-(methylthio)pyrimidin-5-yl)-2,5-dimethylhex-5-en-3-ol (Int 1-7b)

To a solution of 3-[tert-butyl(dimethyl)silyl]oxy-2-(4-chloro-2-methylsulfanyl-pyrimidin-5-yl)-2-methyl-propanal (2 g, 5.54 mmol, 1 eq.) in DCM (50 mL) was added (2-methylallyl) magnesium chloride (0.5 M in THF, 13.6 mL, 1.3 eq.) dropwise at −5° C. The mixture was stirred at 0° C. for 2 h. LCMS and TLC showed two diastereomers. The mixture was quenched by ice/water (50 mL). The aqueous layer was extracted with DCM (2×50 mL). The combined organic layers were washed with brine (2×50 mL), dried over anhydrous Na₂SO₄, filtered and concentrated under reduced pressure. The residue was purified by silica gel column chromatography (eluting with 1:1 EA/PE) to afford rel-(2S,3R)-1-((tert-butyldimethylsilyl)oxy)-2-(4-chloro-2-(methylthio)pyrimidin-5-yl)-2,5-dimethylhex-5-en-3-ol (Int 1-7a) (1.12 g, 2.43 mmol, 48% yield) as a yellow oil (undesired product) and rel-(2S,3S)-1-((tert-butyldimethylsilyl)oxy)-2-(4-chloro-2-(methylthio)pyrimidin-5-yl)-2,5-dimethylhex-5-en-3-ol (Int 1-7b) (900 mg, 1.95 mmol, 39% yield) as a yellow oil (desired product).

rel-(2S,3R)-1-[tert-butyl(dimethyl)silyl]oxy-2-(4-chloro-2-methylsulfanyl-pyrimidin-5-yl)-2,5-dimethyl-hex-5-en-3-ol (Int 1-7a): ¹H NMR (300 MHz, DMSO-d6) δ 8.54 (s, 1H), 4.82-4.78 (m, 1H), 4.70-4.66 (m, 1H), 4.63-4.60 (m, 1H), 4.58-4.52 (m, 1H), 4.46-4.40 (m, 1H), 3.70-3.64 (m, 1H), 2.51 (s, 3H), 2.03-1.93 (m, 1H), 1.73-1.61 (m, 4H), 1.41 (s, 3H), 0.69 (s, 9H), −0.04 (s, 3H), −0.09 (s, 3H). LCMS (ES, m/z): 417, 419[M+H]⁺, Rt 2.124 min.

rel-(2S,3S)-1-((tert-butyldimethylsilyl)oxy)-2-(4-chloro-2-(methylthio)pyrimidin-5-yl)-2,5-dimethylhex-5-en-3-ol (Int 1-7b): ¹H NMR (300 MHz, DMSO-d6) δ 8.56 (s, 1H), 4.75-4.67 (m, 3H), 4.43-4.37 (m, 1H), 4.30-4.25 (m, 1H), 3.77-3.71 (m, 1H), 2.52 (s, 3H), 2.05-1.97 (m, 2H), 1.70 (s, 3H), 1.40 (s, 3H), 0.74 (s, 9H), 0.00 (s, 3H), −0.01 (s, 3H). LCMS (ES, m/z): 417, 419[M+H]⁺, Rt 2.156 min.

### Step 8. rel-(2S,3S)-2-(4-azido-2-methylsulfanyl-pyrimidin-5-yl)-1-[tert-butyl(dimethyl)silyl]oxy-2,5-dimethyl-hex-5-en-3-ol

To a stirred solution of Int 1-7b (1 g, 2.40 mmol, 1 eq.) in DMF (15 mL) was added NaN₃ (450 mg, 5.99 mmol, 2.5 eq.). The mixture was stirred at 60° C. for 18 h. The reaction was monitored by LC-MS. The mixture was allowed to cool down to room temperature. Then ice/water (80 mL) was added. The resulting mixture was extracted with EA (3×80 mL). The organic layers were combined, dried over anhydrous Na₂SO₄, filtered and concentrated under reduced pressure. The residue was purified by silica gel column chromatography (eluting with 1:4 EA/PE) to afford to yield rel-(2S,3S)-2-(4-azido-2-methylsulfanyl-pyrimidin-5-yl)-1-[tert-butyl(dimethyl)silyl]oxy-2,5-dimethyl-hex-5-en-3-ol (800 mg, 1.89 mmol, 79% yield) as a colorless oil. LCMS (ES, m/z): 424.15 [M+H]⁺, Rt 0.892 min.

### Steps 9 and 10. rel-(2S,3S)-2-(4-amino-2-(methylthio)pyrimidin-5-yl)-1-((tert-butyldimethylsilyl)oxy)-2,5-dimethylhex-5-en-3-ol (Intermediate 1)

To a stirred solution of rel-(2S,3S)-2-(4-azido-2-methylsulfanyl-pyrimidin-5-yl)-1-[tert-butyl(dimethyl)silyl]oxy-2,5-dimethyl-hex-5-en-3-ol (900 mg, 2.12 mmol, 1 eq.) in toluene (10 mL) was added PBu₃ (1.57 mL, 6.37 mmol, 3 eq.). The mixture was stirred at 100° C. for 1 h under N₂ atmosphere. The reaction was monitored by LC-MS. The mixture was allowed to cool down to room temperature and was concentrated under reduced pressure to afford the desired tributyl-phosphanylidene intermediate (1.3 g, crude) as a light-yellow oil. LCMS (ES, m/z): 598.55 [M+H]⁺, Rt 1.257 min. To a stirred mixture of the tributyl-phosphanylidene intermediate (1.3 g, 2.17 mmol, 1 eq.) in THF (30 mL) and H₂O (5 mL) was added AcOH (10 mL) slowly. The mixture was stirred at 100° C. for 3 h. The reaction was monitored by LC-MS. The mixture was allowed to cool down to room temperature. The resulting mixture was concentrated under reduced pressure. The pH value was adjusted to 8˜9 by saturated NaHCO₃. The resulting mixture was extracted with EA (3×50 mL). The organic layers were combined, dried over anhydrous Na₂SO₄, filtered and concentrated under reduced pressure. The residue was purified by silica gel column chromatography (eluting with 1:3 EA/PE) to afford Intermediate 1 (700 mg, 1.67 mmol, 77% yield) as a white solid. ¹H NMR (400 MHz, DMSO-d6) δ 7.77 (s, 1H), 7.51 (br, 2H), 6.10 (d, J=5.2 Hz, 1H), 4.75-4.63 (m, 2H), 4.03-4.01 (m, 1H), 3.84-3.77 (m, 1H), 3.61-3.55 (m, 1H), 2.39 (s, 3H), 2.04-1.95 (m, 1H), 1.69-1.60 (m, 4H), 1.30 (s, 3H), 0.76 (s, 9H), −0.05 (s, 3H), −0.16 (s, 3H). LCMS (ES, m/z): 398.15 [M+H]⁺, Rt 0.796 min.

### Intermediate 2: tert-butyl ((6-bromopyridin-2-yl)methyl)(methyl)carbamate

### Step 1. 1-(6-bromopyridin-2-yl)-N-methylmethanamine

To a stirred solution of 6-bromopyridine-2-carbaldehyde (5 g, 26.9 mmol, 1 eq.) and methanamine (2 M in THF, 15 mL, 1.1 eq.) in DCM (50 mL) was added sodium triacetoxyborohydride (6.27 g, 29.6 mmol, 1.1 eq.) in portions at 0° C. The resulting mixture was stirred at r.t. for 12 h. The reaction was monitored by LC-MS. The resulting mixture was washed with brine (2×80 mL) and dried over anhydrous Na₂SO₄. After filtration, the filtrate was concentrated under reduced pressure to afford 1-(6-bromopyridin-2-yl)-N-methylmethanamine (10 g, crude) as a yellow solid. LCMS (ESI+, m/z): 201, 203 [M+H]⁺, Rt 0.289 min.

### Step 2. tert-butyl ((6-bromopyridin-2-yl)methyl)(methyl)carbamate (Intermediate 2)

To a stirred solution of 1-(6-bromo-2-pyridyl)-N-methyl-methanamine (10 g, 49.7 mmol, 1 eq.) and triethylamine (0.12 mol, 17.3 mL, 2.5 eq.) in DCM (100 mL) was added di-tert-butyl dicarbonate (49.7 mmol, 11.4 mL, 1 eq.) dropwise at 0° C. The resulting mixture was stirred at r.t. for 3 h. The reaction was monitored by LC-MS. The mixture was washed with brine (2×80 mL), dried over anhydrous Na₂SO₄. After filtration, the filtrate was concentrated under reduced pressure. The residue was purified by silica gel column chromatography (eluting with 1:1 EA/PE) to afford tert-butyl ((6-bromopyridin-2-yl)methyl)(methyl)carbamate (8 g, 23.9 mmol, 53% yield) as a yellow oil. ¹H NMR (300 MHz, DMSO-d₆) 7.77-7.73 (m, 1H), 7.53 (d, J=7.8 Hz, 1H), 7.25-7.18 (m, 1H), 4.44 (s, 2H), 2.87 (s, 3H), 1.43-1.30 (m, 9H). LCMS (ESI+, m/z): 301, 303 [M+H]⁺, Rt 0.649 min.

The intermediates described herein are commercially available or were synthesized according to the indicated methods below.

TABLE 4
Commercially available intermediates
Structure/Intermediate #

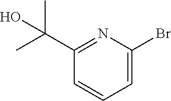

Intermediate 3

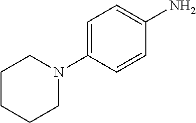 [SMILES: Nc1ccc(N2CCCCC2)cc1]

Intermediate 4

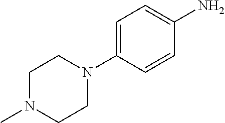

Intermediate 5

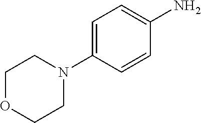 [SMILES: Nc1ccc(N2CCOCC2)cc1]

Intermediate 6

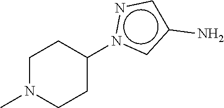

Intermediate 8

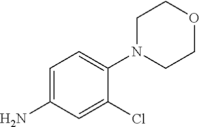 [SMILES: Nc1ccc(N2CCOCC2)c(Cl)c1]

Intermediate 9

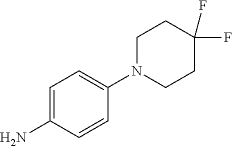 [SMILES: Nc1ccc(N2CCC(F)(F)CC2)cc1]

Intermediate 10

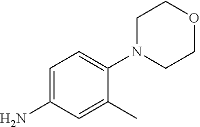

Intermediate 15

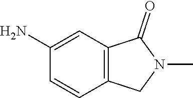

Intermediate 17

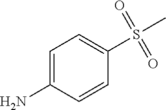

Intermediate 18

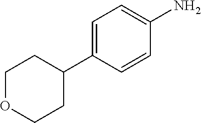 [SMILES: Nc1ccc(C2CCOCC2)cc1]

Intermediate 20

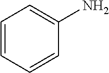

Intermediate 25

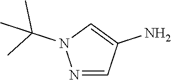

Intermediate 26

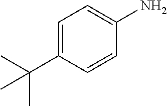

Intermediate 39

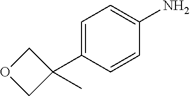

Intermediate 44

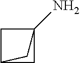

Intermediate 47

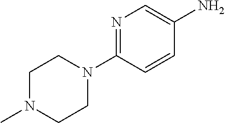

Intermediate 64

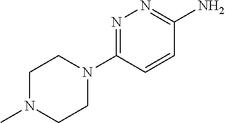

Intermediate 65

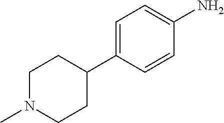

Intermediate 66

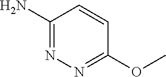

Intermediate 78

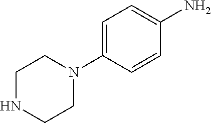 [SMILES: Nc1ccc(N2CCNCC2)cc1]

Intermediate 79 (N-boc)

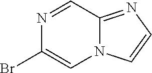

Intermediate 85

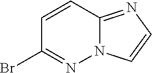

Intermediate 87

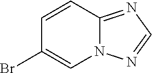

Intermediate 88

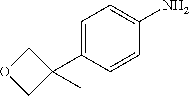

Intermediate 107

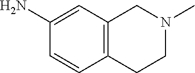

Intermediate 108

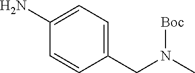 [SMILES: CN(Cc1ccc(N)cc1)C(=O)OC(C)(C)C]

Intermediate 112

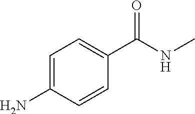

Intermediate 115

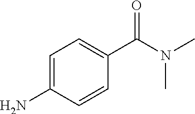

Intermediate 116

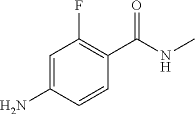

Intermediate 117

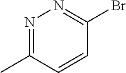

Intermediate 119

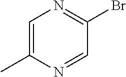

Intermediate 121

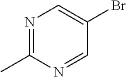

Intermediate 122

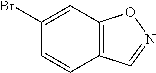

Intermediate 123

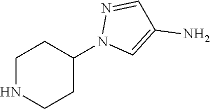 [SMILES: Nc1cnn(C2CCNCC2)c1]

Intermediate 125

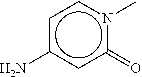

Intermediate 143

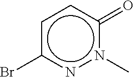

Intermediate 150

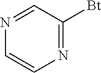

Intermediate 151

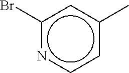

Intermediate 152

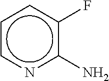

Intermediate 153

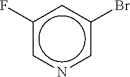

Intermediate 154

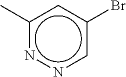

Intermediate 155

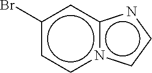

Intermediate 156

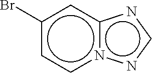

Intermediate 157

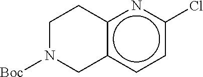

Intermediate 209

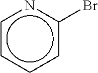

Intermediate 164

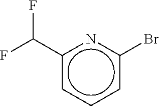

Intermediate 194

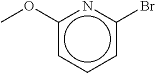

Intermediate 196

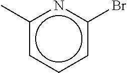

Intermediate 197

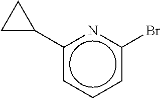

Intermediate 198

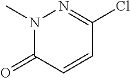

Intermediate 201

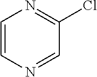

Intermediate 203

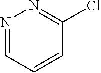

Intermediate 204

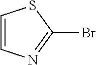

Intermediate 214

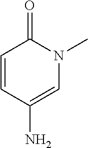

Intermediate 255

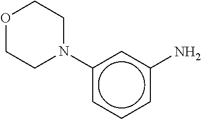

Intermediate 257

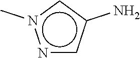

Intermediate 260

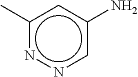

Intermediate 155

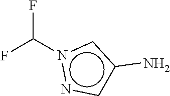

Intermediate 261

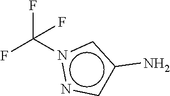

Intermediate 262

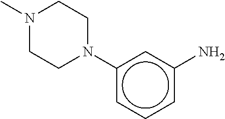

Intermediate 258

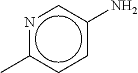

Intermediate 265

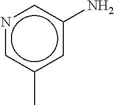

Intermediate 266

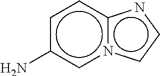

Intermediate 268

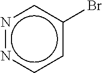

Intermediate 271

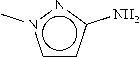

Intermediate 272

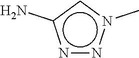

Intermediate 276

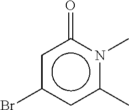

Intermediate 274

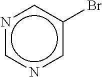

Intermediate 275

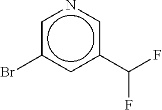

Intermediate 276

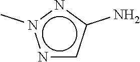

Intermediate 278

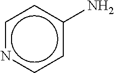

Intermediate 279

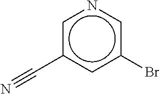

Intermediate 280

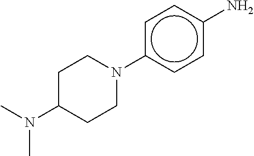

Intermediate 281

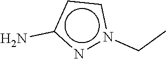

Intermediate 282

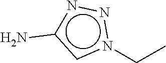

Intermediate 283

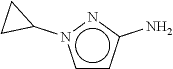

Intermediate 284

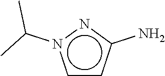

Intermediate 285

### Intermediate 7: 3-methyl-4-(4-methylpiperazin-1-yl)aniline

### Step 1. 1-methyl-4-(2-methyl-4-nitrophenyl)piperazine

To a stirred mixture of 1-fluoro-2-methyl-4-nitrobenzene (5.0 g, 32.23 mmol, 1 eq.) and 1-methylpiperazine (3.87 g, 38.68 mmol, 4.29 mL, 1.20 eq.) in DMF (20 mL) was added K₂CO₃ (9.90 g, 64.46 mmol, 2 eq.). The resulting mixture was stirred for 3 h at 90° C. The reaction was monitored by LC-MS. The mixture was allowed to cool down to room temperature. The resulting mixture was diluted with H₂O (100 mL). The resulting mixture was extracted with EA (3×100 mL). The combined organic layers were washed with brine (2×200 mL), dried over anhydrous Na₂SO₄. After filtration, the filtrate was concentrated under reduced pressure. The residue was purified by silica gel column chromatography (eluting with 1:1 EA/PE) to afford 1-methyl-4-(2-methyl-4-nitrophenyl)piperazine (5.1 g, 67% yield) as a yellow solid. LCMS (ESI+, m/z): 236 [M+H]⁺, Rt 0.553 min.

### Step 2. 3-methyl-4-(4-methylpiperazin-1-yl)aniline (Intermediate 7)

To a stirred solution of 1-methyl-4-(2-methyl-4-nitrophenyl)piperazine (5.1 g, 21.68 mmol, 1 eq) in MeOH (50 mL) was added Pd/C (200 mg, 10 wt %) at room temperature under N₂ atmosphere. The resulting mixture was stirred for 5 h at room temperature under H₂ atmosphere. The solids were filtered and washed with MeOH (3×15 mL). The filtrate was concentrated under reduced pressure. The residue was purified by silica gel column chromatography (eluting with 10:1 DCM/MeOH) to afford 3-methyl-4-(4-methylpiperazin-1-yl)aniline (2.7 g, 61% yield) as a yellow solid. LCMS (ESI+, m/z): 206 [M+H]⁺, Rt 0.305 min.

TABLE 5
The following intermediates were synthesized according to the procedure for
Intermediate 7.

| Structure/Intermediate # | LCMS |
|---|---|
| 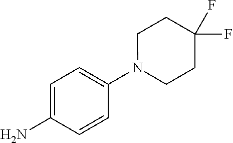 [SMILES: Nc1ccc(N2CCC(F)(F)CC2)cc1] | LCMS (ES, m/z): 213 [M + H]⁺, Rt 0.555 min |
| Intermediate 10 | |
| 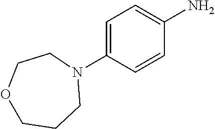 [SMILES: Nc1ccc(N2CCCOCC2)cc1] | LCMS (ES, m/z): 193 [M + H]⁺, Rt 0.375 min |
| Intermediate 11 | |
| 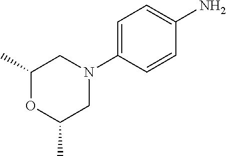 | LCMS (ES, m/z): 207 [M + H]⁺, Rt 0.660 min. |
| Intermediate 12 | |
| 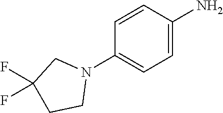 [SMILES: Nc1ccc(N2CCC(F)(F)C2)cc1] | LCMS (ES, m/z): 199 [M + H]⁺, Rt 0.533 min |
| Intermediate 13 | |
| 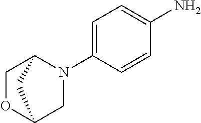 [SMILES: Nc1ccc(N2C[C@@H]3C[C@H]2CO3)cc1] | LCMS (ES, m/z): 191 [M + H]⁺, Rt 0.261 min |
| Intermediate 23 | |
| 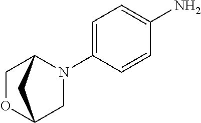 [SMILES: Nc1ccc(N2C[C@H]3C[C@@H]2CO3)cc1] | LCMS (ES, m/z): 191 [M + H]⁺, Rt 0.255 min |
| Intermediate 24 | |
| 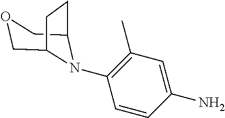 [SMILES: Cc1cc(N)ccc1N1C2CCC1COC2] | LCMS (ES, m/z): 219 [M + H]⁺, Rt 0.499 min. |
| Intermediate 45 | |
| 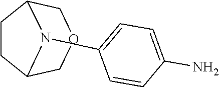 [SMILES: Nc1ccc(N2C3CCC2COC3)cc1] | LCMS (ES, m/z): 205 [M + H]⁺, Rt 0.401 min. |
| Intermediate 46 | |
| 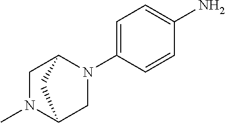 | LCMS (ES, m/z): 204 [M + H]⁺; RT: 0.499 min. |
| Intermediate 54 | |
| 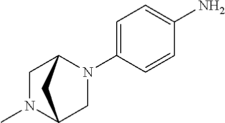 | LCMS (ES, m/z): 204 [M + H]⁺, Rt 0.162 min |
| Intermediate 55 | |
| 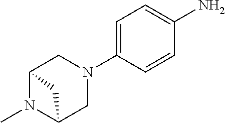 | LCMS (ES, m/z): 290 [M + H]⁺; RT: 0.450 min. |
| Intermediate 60 | |
| 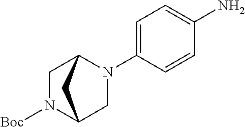 [SMILES: CC(C)(C)OC(=O)N1C[C@H]2C[C@@H]1CN2c1ccc(N)cc1] | LCMS (ES, m/z): 290 [M + H]⁺, Rt 0.583 min. |
| Intermediate 80 | |
| 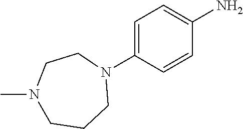 | LCMS (ES, m/z): 206 [M + H]⁺, Rt 0.098 min. |
| Intermediate 81 | |
| 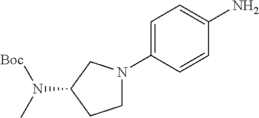 [SMILES: CN(C(=O)OC(C)(C)C)[C@H]1CCN(c2ccc(N)cc2)C1] | LCMS (ES, m/z): 292 [M + H]⁺, Rt 0.723 min. |
| Intermediate 82 | |
| 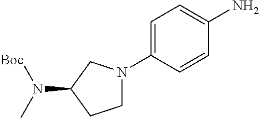 [SMILES: CN(C(=O)OC(C)(C)C)[C@@H]1CCN(c2ccc(N)cc2)C1] | LCMS (ES, m/z): 292 [M + H]⁺, Rt 0.722 min |
| Intermediate 83 | |
| 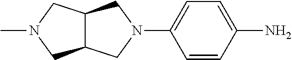 [SMILES: CN1C[C@@H]2CN(c3ccc(N)cc3)C[C@@H]2C1] | LCMS (ES, m/z): 218 [M + H]⁺, Rt 0.857 min |
| Intermediate 84 | |
| 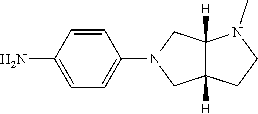 [SMILES: CN1CC[C@@H]2CN(c3ccc(N)cc3)C[C@@H]21] | LCMS (ES, m/z): 218 [M + H]⁺, Rt 0.588 min. |
| Intermediate 230 | |
| 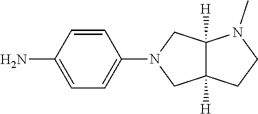 [SMILES: CN1CC[C@H]2CN(c3ccc(N)cc3)C[C@H]21] | LCMS (ES, m/z): 218 [M + H]⁺, Rt 0.133min. |
| Intermediate 231 | |
| 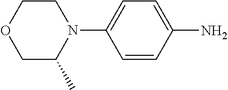 | LCMS (ES, m/z): 193 [M + H]⁺, Rt 0.840 min. |
| Intermediate 232 | |
| 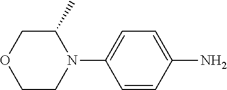 | LCMS (ES, m/z): 193 [M + H]⁺, Rt 0.302 min. |
| Intermediate 233 | |
| 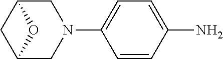 [SMILES: Nc1ccc(N2C[C@H]3C[C@@H](C2)O3)cc1] | LCMS (ES, m/z): 191 [M + H]⁺, Rt 0.3 min. |
| Intermediate 234 | |
| 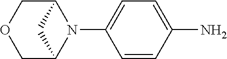 [SMILES: Nc1ccc(N2[C@@H]3COC[C@H]2C3)cc1] | LCMS (ES, m/z): 191 [M + H]⁺, Rt 0.400 min. |
| Intermediate 235 | |
| 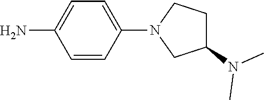 | LCMS (ES, m/z): 206 [M + H]⁺, Rt 0.154 min. |
| Intermediate 236 | |
| 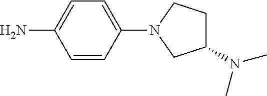 | LCMS (ES, m/z): 206 [M + H]⁺, Rt 0.870 min. |
| Intermediate 237 | |

### Intermediate 16: (S)-1-(4-aminophenyl)-5-methylpyrrolidin-2-one

### Step 1. (S)-5-methyl-1-(4-nitrophenyl)pyrrolidin-2-one

A solution of (S)-5-methylpyrrolidin-2-one (800 mg, 8.07 mmol, 1 eq), 1-bromo-4-nitrobenzene (1.96 g, 9.68 mmol, 1.00 mL, 1.2 eq), Pd₂(dba)₃ (739 mg, 807.02 μmol, 0.1 eq), Xantphos (385 mg, 807 μmol, 0.1 eq) and Cs₂CO₃ (5.26 g, 16.14 mmol, 2 eq) in dioxane (10 mL) was stirred for 2 h at 100° C. under N₂ atmosphere. The reaction was monitored by LCMS. The mixture was allowed to cool down to room temperature. The solids were filtered out and washed with EA (2×50 mL). The filtrate was concentrated under reduced pressure. The residue was purified by silica gel column chromatography (eluting with 1:2 EA/PE) to afford (S)-5-methyl-1-(4-nitrophenyl)pyrrolidin-2-one (1.5 g, 84% yield) as yellow solid. LCMS (ESI+, m/z): 221 [M+H]⁺, Rt 0.620 min.

### Step 2. (S)-1-(4-aminophenyl)-5-methylpyrrolidin-2-one (Intermediate 16)

To a stirred solution of (S)-5-methyl-1-(4-nitrophenyl)pyrrolidin-2-one (1.0 g, 4.54 mmol) in MeOH (15 mL) and EA (5 mL) was added Pd/C (551 mg, 10 wt %) under N₂ atmosphere. The mixture was stirred for 24 h at room temperature under H₂ atmosphere. The reaction was monitored by LCMS. The solids were filtered and washed with MeOH (2×30 mL). The filtrate was concentrated under reduced pressure. The residue was purified by silica gel column chromatography (eluting with 10:1 DCM/MeOH) to afford (S)-1-(4-aminophenyl)-5-methyl-pyrrolidin-2-one (700 mg, 81% yield) as a yellow solid. ¹H NMR (300 MHz, DMSO-d6) δ 6.99-6.92 (m, 2H), 6.59-6.52 (m, 2H), 5.06 (br, 2H), 4.17-4.04 (m, 1H), 2.49-2.18 (m, 3H), 1.71-1.53 (m, 1H), 1.05 (d, J=6.0 Hz, 3H). LCMS (ESI+, m/z): 191 [M+H]⁺, Rt 0.403 min.

### Intermediate 19: (R)-1-(4-aminophenyl)-5-methylpyrrolidin-2-one

### Step 1. (R)-5-methyl-1-(4-nitrophenyl)pyrrolidin-2-one

A solution of (R)-5-methylpyrrolidin-2-one (450 mg, 4.54 mmol, 1 eq.), 1-bromo-4-nitrobenzene (1.10 g, 5.45 mmol, 1.2 eq.), Pd₂(dba)₃ (416 mg, 454 μmol, 0.1 eq.), Xantphos (216 mg, 454 μmol, 0.1 eq.) and Cs₂CO₃ (2.96 g, 9.08 mmol, 2 eq.) in dioxane (5 mL) was stirred for 2 h at 100° C. under N₂ atmosphere. The reaction was monitored by LC-MS. The mixture was allowed to cool down to room temperature. The solids were filtered and washed with EA (2×10 mL). The filtrate was concentrated under reduced pressure. The residue was purified by silica gel column chromatography (eluting with 1:2 EA/PE) to afford (R)-5-methyl-1-(4-nitrophenyl)pyrrolidin-2-one (950 mg, 95% yield) as yellow solid. LCMS (ESI+, m/z): 221 [M+H]⁺, Rt 0.637 min.

### Step 2. (R)-1-(4-aminophenyl)-5-methylpyrrolidin-2-one (Intermediate 19)

To a stirred solution of (R)-5-methyl-1-(4-nitrophenyl)pyrrolidin-2-one (0.95 g, 4.31 mmol, 1 eq.) in MeOH (6 mL) and EA (6 mL) was added Pd/C (100 mg, 10 wt %). The mixture was stirred for 24 h at room temperature under H₂ atmosphere. The reaction was monitored by LC-MS. The solids were filtered and washed with MeOH (3×20 mL). The filtrate was concentrated under reduced pressure. The residue was purified by silica gel column chromatography (eluting with 10:1 DCM/MeOH) to afford (R)-1-(4-aminophenyl)-5-methyl-pyrrolidin-2-one (750 mg, 92% yield) as a yellow solid. ¹H NMR (300 MHz, DMSO-d₆) δ 6.99-6.91 (m, 2H), 6.59-6.51 (m, 2H), 5.06 (br, 2H), 4.20-3.97 (m, 1H), 2.49-2.17 (m, 3H), 1.70-1.53 (m, 1H), 1.05 (d, J=6.0 Hz, 3H). LCMS (ESI+, m/z): 191 [M+H]⁺, Rt 0.403 min.

### Intermediates 21 and 22: (S)-4-(tetrahydrofuran-3-yl)aniline and (R)-4-(tetrahydrofuran-3-yl)aniline

### Step 1. 3-(4-nitrophenyl)-2,5-dihydrofuran

A mixture of 1-bromo-4-nitrobenzene (800 mg, 3.96 mmol, 1 eq.), 2-(2,5-dihydrofuran-3-yl)-4,4,5,5-tetramethyl-1,3,2-dioxaborolane (1.16 g, 5.94 mmol, 1.5 eq.), Pd(dppf)Cl₂ (323 mg, 396 μmol, 0.1 eq.) and Na₂CO₃ (839 mg, 7.92 mmol, 2 eq.) in mixed solvent of H₂O (4 mL) and dioxane (16 mL) was irradiated with microwave at 130° C. for 30 min under N₂ atmosphere. The reaction was monitored by LC-MS. The mixture was allowed to cool down to room temperature. The resulting mixture was diluted with brine (30 mL). The resulting mixture was extracted with EA (3×30 mL). The combined organic layers were dried over anhydrous Na₂SO₄. After filtration, the filtrate was concentrated under reduced pressure. The residue was purified by silica gel column chromatography (eluting with 1:3 EA/PE) to afford 3-(4-nitrophenyl)-2,5-dihydrofuran (450 mg, 59% yield) as a yellow solid. LCMS (ESI+, m/z): 192 [M+H]⁺, Rt 0.673 min.

### Step 2. 4-(tetrahydrofuran-3-yl)aniline

To a stirred mixture of 3-(4-nitrophenyl)-2,5-dihydrofuran (450 mg, 2.35 mmol, 1 eq.) in MeOH (10 mL) was added Pd/C (90 mg, 10 wt %) under N₂ atmosphere. The resulting mixture was stirred for 2 h at room temperature under H₂ atmosphere. The reaction was monitored by LC-MS. The solids were filtered and washed with EA (3×20 ml). The filtrate was concentrated under reduced pressure. The residue was purified by silica gel column chromatography (eluting with 1:5 EA/PE) to afford 4-(tetrahydrofuran-3-yl)aniline (320 mg, 83% yield) as brown solid. ¹H NMR (300 MHz, DMSO-d₆) δ 6.93 (d, J=8.4 Hz, 2H), 6.51 (d, J=8.4 Hz, 2H), 4.90 (s, 2H), 4.01-3.84 (m, 2H), 3.80-3.72 (m, 1H), 3.45-3.38 (m, 1H), 3.23-3.12 (m, 1H), 2.26-2.14 (m, 1H), 1.89-1.76 (m, 1H). LCMS (ESI+, m/z): 164 [M+H]⁺, Rt 0.132 min.

### Step 3. Separation of enantiomers to obtain (S)-4-(tetrahydrofuran-3-yl)aniline and (R)-4-(tetrahydrofuran-3-yl)aniline (Intermediates 21 and 22)

The racemic compound 4-(tetrahydrofuran-3-yl)aniline (320 mg, 1.96 mmol, 1 eq.) was separated by Prep-Chiral-HPLC with the following conditions (Column: CHIRALPAK IH, 2×25 cm, 5 μm; Mobile Phase A: Hex (0.5% 2 M NH₃-MeOH)-HPLC, Mobile Phase B: IPA-HPLC; Flow rate: 20 mL/min; Gradient: 80% B to 80% B in 20 min; Wave Length: 220/254 nm; RT1(min): 12.481; RT2(min): 15.848). The first eluting isomer was isolated to afford Intermediate 21 (150 mg, 47% yield) as a yellow solid. The second eluting isomer was isolated to afford Intermediate 22 (140 mg, 44% yield) as a yellow solid.

### Intermediates 28 and 29: (S)-5-(4-aminophenyl)-1-methylpyrrolidin-2-one and (R)-5-(4-aminophenyl)-1-methylpyrrolidin-2-one

### Step 1. 5-(4-bromophenyl)-1-methylpyrrolidin-2-one

To a solution of 5-(4-bromophenyl)pyrrolidin-2-one (1.00 g, 4.16 mmol, 1 eq.) in DMF (15 mL) was added NaH (60% suspension in mineral oil, 144 mg, 6.25 mmol, 1.5 eq.) at 0° C. The mixture was stirred for 15 min at room temperature. Then Mel (709 mg, 5.00 mmol, 1.2 eq.) was added. The mixture was allowed to warm to room temperature and stirred for 2 h. The reaction was monitored by LC-MS. The reaction mixture was quenched by water (50 mL) and extracted with EA (3×50 mL). The filtrate was concentrated under reduced pressure. The residue was purified by silica gel column chromatography (eluting with 1:3 EA/PE) to afford 5-(4-bromophenyl)-1-methyl-pyrrolidin-2-one (900 mg, 85% yield) as a yellow oil. LCMS (ESI+, m/z): 254 [M+H]⁺, Rt 1.118 min.

### Step 2. 5-(4-((diphenylmethylene)amino)phenyl)-1-methylpyrrolidin-2-one

To a solution of 5-(4-bromophenyl)-1-methyl-pyrrolidin-2-one (900 mg, 3.54 mmol, 1 eq.), diphenylmethanimine (770 mg, 4.25 mmol, 1.2 eq.), BrettPhos (190 mg, 354 μmol, 0.1 eq.) and Cs₂CO₃ (3.46 g, 10.62 mmol, 3 eq.) in 1,4-dioxane (8 mL) was added BrettPhos-Pd-G3 (321 mg, 354 μmol, 0.1 eq.) at room temperature under N₂ atmosphere. The resulting mixture was stirred for 3 h at 100° C. under N₂ atmosphere. The reaction was monitored by LCMS. The mixture was allowed to cool down to room temperature. The resulting mixture was filtered and the filter cake was washed with DCM (3×10 mL). The filtrate was concentrated under reduced pressure. The residue was purified by silica gel column chromatography (eluting with 1:10 MeOH/DCM) to afford 5-(4-((diphenylmethylene)amino)phenyl)-1-methylpyrrolidin-2-one (905 mg, 72% yield) as a yellow solid. LCMS (ESI+, m/z): 355 [M+H]⁺, Rt 0.978 min.

### Step 3. 5-(4-aminophenyl)-1-methylpyrrolidin-2-one

To a solution of 5-(4-((diphenylmethylene)amino)phenyl)-1-methylpyrrolidin-2-one (905 mg, 2.55 mmol) in THF (6 mL) were added H₂O (1 mL) and AcOH (2 mL). The resulting mixture was stirred for 1 h at 100° C. The reaction was monitored by LC-MS. The mixture was allowed to cool down to room temperature. The reaction was quenched by saturated aqueous NaHCO₃ (50 mL). The resulting mixture was extracted with EA (3×50 mL). The combined organic layers were washed with brine (2×100 mL), and dried over anhydrous Na₂SO₄. After filtration, the filtrate was concentrated under reduced pressure. The residue was purified by silica gel column chromatography (eluting with 1:10 MeOH/DCM) to afford 5-(4-aminophenyl)-1-methylpyrrolidin-2-one (420 mg, 87% yield) as a yellow solid. LCMS (ESI+, m/z): 191 [M+H]⁺, Rt 0.320 min. ¹H NMR (300 MHz, DMSO-d6) δ 6.92-6.87 (m, 2H), 6.58-6.54 (m, 2H), 5.08 (br, 2H), 4.37-4.32 (m, 1H), 2.45 (s, 3H), 2.42-2.20 (m, 3H), 1.80-1.69 (m, 1H). LCMS (ES, m/z): 191[M+H]⁺, Rt 0.320 min.

### Step 4. Separation of enantiomers to obtain (S)-5-(4-aminophenyl)-1-methylpyrrolidin-2-one and (R)-5-(4-aminophenyl)-1-methylpyrrolidin-2-one (Intermediates 28 and 29)

The racemic product 5-(4-Aminophenyl)-1-methyl-pyrrolidin-2-one (850 mg, 4.47 mmol, 1 eq.) was separated by prep-Chiral-HPLC with the following conditions (Column: EnantioPak A1-5, 2.12×25 cm, 5 μm; Mobile Phase A: CO₂, Mobile Phase B: MeOH (0.1% 2M NH₃-MeOH); Flow rate: 50 mL/min; Gradient: isocratic 35% B; Column Temperature: 35° C.; Back Pressure: 100 bar; Wave Length: 220 nm; RT1(min): 3.4; RT2(min): 4.61; Sample Solvent: MeOH (0.1% 2M NH₃-MeOH) to afford Intermediate 28 (350 mg, 41% yield) and Intermediate 29 (330 mg, 39% yield).

### Intermediate 30: 6-(tetrahydro-2H-pyran-4-yl)pyridin-3-amine

### Step 1. 2-(3,6-dihydro-2H-pyran-4-yl)-5-nitropyridine

A mixture of 2-bromo-5-nitropyridine (2 g, 9.85 mmol, 1 eq.), 2-(3,6-dihydro-2H-pyran-4-yl)-4,4,5,5-tetramethyl-1,3,2-dioxaborolane (4.14 g, 19.71 mmol, 2 eq.), KOAc (8.34 g, 29.56 mmol, 3 eq.) and Pd(dppf)C12 (805 mg, 985 μmol, 0.1 eq.) in 1,4-dioxane (20 mL) and H₂O (5 mL) was stirred for 2 h at 100° C. under N₂ atmosphere. The reaction was monitored by LCMS. The mixture was allowed to cool down to room temperature. Then water (30 mL) was added. The mixture was extracted with EA (3×30 mL). The combined organic layers were washed with brine (2×30 mL), dried over anhydrous Na₂SO₄, filtered and concentrated. The residue was purified by silica gel column chromatography (eluting with 1:1 EA/PE) to afford 2-(3,6-dihydro-2H-pyran-4-yl)-5-nitropyridine (900 mg, 44% yield) as a yellow solid. LCMS (ESI+, m/z): 207 [M+H]⁺, Rt 0.623 min.

### Step 2. 6-(tetrahydro-2H-pyran-4-yl)pyridin-3-amine (Intermediate 30)

To a stirred solution of 2-(3,6-dihydro-2H-pyran-4-yl)-5-nitropyridine (550 mg, 2.67 mmol, 1 eq.) in EtOH (10 mL) and EA (10 mL) was added Pd/C (110 mg, 10 wt %) under N₂ atmosphere. The resulting mixture was stirred for 2 h at room temperature under H₂ atmosphere. The reaction was monitored by LCMS. The resulting mixture was filtered. The filter cake was washed with MeOH (3×10 mL). The filtrate was concentrated and purified by silica gel column chromatography (eluting with 1:10 MeOH/DCM) to afford 6-(tetrahydro-2H-pyran-4-yl)pyridin-3-amine (420 mg, 76% yield) as a yellow solid. ¹H NMR (300 MHz, DMSO-d₆) δ 7.86 (s, 1H), 6.96-6.82 (m, 2H), 5.10 (br, 2H), 4.02-3.80 (m, 2H), 3.49-3.30 (m, 2H), 2.78-2.60 (m, 1H), 1.74-1.54 (m, 4H). LCMS (ESI+, m/z): 179 [M+H]⁺, Rt 0.385 min.

TABLE 6
The following intermediates were prepared in an analogous manner as
intermediate 30.

| Structure/Intermediate # | LCMS |
|---|---|
| 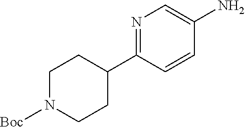 [SMILES: CC(C)(C)OC(=O)N1CCC(c2ccc(N)cn2)CC1] | LCMS (ES, m/z): 278 [M + H]⁺, Rt 0.583 min. |
| Intermediate 67 | |
| 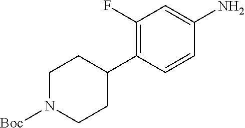 [SMILES: CC(C)(C)OC(=O)N1CCC(c2ccc(N)cc2F)CC1] | LCMS (ES, m/z): 239 [M + H − 56]⁺, Rt 0.667 min. |
| Intermediate 68 | |
| 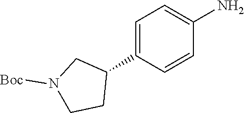 [SMILES: CC(C)(C)OC(=O)N1CC[C@@H](c2ccc(N)cc2)C1] | LCMS (ES, m/z): 207 [M + H − 56]⁺, Rt 0.553 min. |
| Intermediate 89 | |
| 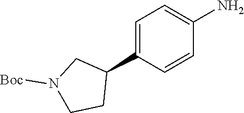 [SMILES: CC(C)(C)OC(=O)N1CC[C@H](c2ccc(N)cc2)C1] | LCMS (ES, m/z): 207 [M + H − 56]⁺, Rt 0.553 min. |
| Intermediate 90 | |
| 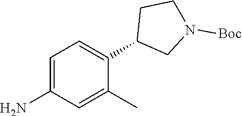 [SMILES: Cc1cc(N)ccc1[C@@H]1CCN(C(=O)OC(C)(C)C)C1] | LCMS (ES, m/z): 221 [M + H − 56]⁺, Rt 0.716 min. |
| Intermediate 91 | |
| 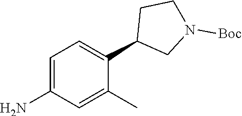 [SMILES: Cc1cc(N)ccc1[C@H]1CCN(C(=O)OC(C)(C)C)C1] | LCMS (ES, m/z): 221 [M + H − 56]⁺, Rt 0.716 min. |
| Intermediate 92 | |
| 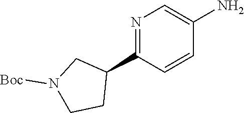 [SMILES: CC(C)(C)OC(=O)N1CC[C@H](c2ccc(N)cn2)C1] | LCMS (ES, m/z): 264 [M + H]⁺, Rt 0.477 min. |
| Intermediate 93 | |
| 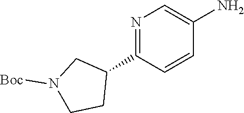 [SMILES: CC(C)(C)OC(=O)N1CC[C@@H](c2ccc(N)cn2)C1] | LCMS (ES, m/z): 264 [M + H]⁺, Rt 0.477 min. |
| Intermediate 94 | |
| 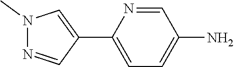 | LCMS (ES, m/z): 175 [M + H]⁺, Rt 0.468 min. |
| Intermediate 238 | |
| 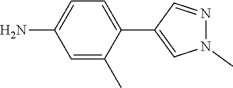 | LCMS (ES, m/z): 188 [M + H]⁺, Rt 0.389 min. |
| Intermediate 239 | |

### Intermediate 31 and 32: (R)-4-(tetrahydrofuran-2-yl)aniline and (S)-4-(tetrahydrofuran-2-yl)aniline

### Step 1. 5-(4-nitrophenyl)-2,3-dihydrofuran

A mixture of 1-bromo-4-nitro-benzene (1.50 g, 7.43 mmol, 1 eq.), Pd₂(dba)₃ (680 mg, 743 μmol, 0.1 eq.), sodium formate (656 mg, 9.65 mmol, 1.3 eq.), TBAC (3.10 g, 11.14 mmol, 1.5 eq.) and 2,3-dihydrofuran (2.86 g, 40.84 mmol, 5.5 eq.) in DMF (80 mL) was stirred for 16 h at room temperature. The reaction was monitored by TLC. The resulting mixture was filtered through a Celite pad and washed with EA (3×20 mL). The filtrate was concentrated under reduced pressure. The residue was purified by silica gel column chromatography (eluting with 1:3 EA/PE) to afford 5-(4-nitrophenyl)-2,3-dihydrofuran (880 mg, 62% yield) as a yellow oil.

### Step 2. 4-(tetrahydrofuran-2-yl)aniline

To a solution of 5-(4-nitrophenyl)-2,3-dihydrofuran (1.20 g, 6.28 mmol) in EtOH (50 mL) was added Pd/C (120 mg, 10 wt %) under N₂ atmosphere. The resulting mixture was stirred for 1 h at room temperature under H₂ atmosphere. The reaction was monitored by LC-MS. The resulting mixture was filtered through a Celite pad and washed with MeOH (2×10 mL). The filtrate was concentrated under reduced pressure. The residue was purified by silica gel column chromatography (eluting with 1:1 EA/PE) to afford 4-(tetrahydrofuran-2-yl)aniline (700 mg, 69% yield) as a yellow oil. ¹H NMR (300 MHz, DMSO-d6) δ 6.98-6.89 (m, 2H), 6.54-6.44 (m, 2H), 4.96 (br, 2H), 4.58 (t, J=6.0 Hz, 1H), 3.95-3.88 (m, 1H), 3.75-3.68 (m, 1H), 2.20-2.09 (m, 1H), 1.99-1.85 (m, 2H), 1.69-1.57 (m, 1H). LCMS (ESI+, m/z): 164 [M+H]⁺, Rt 0.942 min.

### Step 3. Separation of enantiomers to obtain (R)-4-(tetrahydrofuran-2-yl)aniline and (S)-4-(tetrahydrofuran-2-yl)aniline (Intermediates 31 and 32)

4-(Tetrahydrofuran-2-yl)aniline (700 mg, 4.29 mmol, 1 eq.) was separated by chiral-HPLC by the following conditions (Column: Exsil Chiral-NR, 3×25 cm, 8 μm; Mobile Phase A: CO₂, Mobile Phase B: IPA (0.5% 2M NH₃-MeOH); Flow rate: 80 mL/min; Gradient: isocratic 40% B; Column Temperature: 35° C.; Back Pressure: 100 bar; Wave Length: 220 nm; RT1(min): 3.95; RT2(min): 5.65; Sample Solvent: MeOH—Preparative; Injection Volume: 4.8 mL; Number Of Runs: 4). The first eluting isomer was isolated to afford Intermediate 31 (157 mg) as a yellow oil. The second eluting isomer was isolated to afford Intermediate 32 (150 mg) as a yellow oil.

### Intermediates 33 and 34: tert-butyl (S)-4-(4-aminophenyl)-2-oxo-pyrrolidine-1-carboxylate and tert-butyl (R)-4-(4-aminophenyl)-2-oxo-pyrrolidine-1-carboxylate

### Step 1. tert-butyl 2-hydroxy-4-(4-nitrophenyl)pyrrolidine-1-carboxylate

A mixture of 4-nitrobenzenediazonium tetrafluoroborate (5 g, 21.10 mmol, 1 eq), tert-butyl 2,5-dihydropyrrole-1-carboxylate (3.57 g, 21.10 mmol, 1 eq) and palladium(II) acetate (474 mg, 2.11 mmol, 0.1 eq) in ACN (30 mL) was stirred for 3 h at room temperature under N₂ atmosphere. The reaction was monitored by LC-MS. The reaction mixture was diluted with H₂O (100 mL). The resulting mixture was extracted with EA (3×100 mL). The combined organic layers were washed with brine (2×200 mL), dried over anhydrous Na₂SO₄. After filtration, the filtrate was concentrated under reduced pressure to afford tert-butyl 2-hydroxy-4-(4-nitrophenyl)pyrrolidine-1-carboxylate (5 g, crude), which was used in the next step directly without further purification. LCMS (ESI+, m/z): 309 [M+H]⁺, Rt 0.967 min.

### Step 2. tert-butyl 4-(4-nitrophenyl)-2-oxo-pyrrolidine-1-carboxylate (Intermediate 33/34-2)

To a stirred solution of tert-butyl 2-hydroxy-4-(4-nitrophenyl)pyrrolidine-1-carboxylate (5 g, 16.22 mmol, 1 eq.) in DCM (20 mL) was added pyridinium chlorochromate (6.99 g, 32.43 mmol, 2 eq.) at 0° C. The resulting mixture was stirred for 2 h at room temperature. The reaction was monitored by LC-MS. The solids were filtered out and washed with DCM (3×50 mL). The filtrate was concentrated under reduced pressure. The residue was purified by silica gel column chromatography (eluting with 1:1 EA/PE) to afford tert-butyl 4-(4-nitrophenyl)-2-oxo-pyrrolidine-1-carboxylate (Intermediate 33/34-2) (2.7 g, 54% yield) as a yellow oil. LCMS (ESI+, m/z): 307 [M+H]⁺, Rt 0.942 min.

### Step 3. tert-butyl 4-(4-aminophenyl)-2-oxo-pyrrolidine-1-carboxylate

To a stirred mixture of tert-butyl 4-(4-nitrophenyl)-2-oxo-pyrrolidine-1-carboxylate (1.2 g, 3.92 mmol, 1 eq) in EA (10 mL) and EtOH (10 mL) was added Pd/C (120 mg, 10 wt %) under N₂ atmosphere. The resulting mixture was stirred for 2 h at room temperature under H₂ atmosphere. The reaction was monitored by LC-MS. The solids were filtered out and washed with EtOH (2×15 mL). The filtrate was concentrated under reduced pressure. The residue was purified by silica gel column chromatography (eluting with 10:1 DCM/MeOH) to afford tert-butyl 4-(4-aminophenyl)-2-oxo-pyrrolidine-1-carboxylate (700 mg, 65% yield) as a yellow oil. ¹H NMR (300 MHz, DMSO-d₆) δ 6.97 (d, J=8.4 Hz, 2H), 6.52 (d, J=8.4 Hz, 2H), 4.98 (br, 2H), 4.01-3.90 (m, 1H), 3.51-3.42 (m, 1H), 3.40-3.30 (m, 1H), 2.66-2.59 (m, 2H), 1.45 (s, 9H). LCMS (ESI+, m/z): 277 [M+H]⁺, Rt 0.553 min.

### Step 4. Separation of Enantiomers to Obtain tert-butyl (S)-4-(4-aminophenyl)-2-oxo-pyrrolidine-1-carboxylate and tert-butyl (R)-4-(4-aminophenyl)-2-oxo-pyrrolidine-1-carboxylate (Intermediates 33 and 34)

The racemic compound tert-butyl 4-(4-aminophenyl)-2-oxo-pyrrolidine-1-carboxylate (700 mg, 2.52 mmol, 1 eq) was separation by Prep-Chiral-HPLC with the following conditions (Column: CHIRALPAK IF, 3×25 cm, 5 μm; Mobile Phase A: CO₂, Mobile Phase B: MeOH (0.1% 2M NH₃-MeOH); Flow rate: 70 mL/min; Gradient: isocratic 35% B; Column Temperature: 35° C.; Back Pressure: 100 bar; Wave Length: 220 nm; RT1(min): 6.13; RT2(min): 8.14; Sample Solvent: MeOH—Preparative; Injection Volume: 4.8 mL; Number Of Runs: 5). The first eluting isomer was isolated to afford Intermediate 33 (300 mg) as a yellow solid. The second eluting isomer was isolated to afford Intermediate 34 (310 mg) as a yellow solid.

### Intermediates 35 and 36: (R)-4-(4-aminophenyl)-1-methyl-pyrrolidin-2-one and (S)-4-(4-aminophenyl)-1-methyl-pyrrolidin-2-one

### Step 1. 4-(4-nitrophenyl)pyrrolidin-2-one

To a solution of Intermediate 33/34-2 (2.9 g, 9.47 mmol, 1 eq.) in DCM (30 mL) was added TFA (5 mL) at 0° C. The resulting mixture was stirred for 1 h at room temperature. The reaction was monitored by LC-MS. The mixture was basified to pH=8 with NaHCO₃(aq). The resulting mixture was extracted with EA (3×50 mL). The combined organic layers were washed with brine (2×50 mL), dried over anhydrous Na₂SO₄. After filtration, the filtrate was concentrated under reduced pressure. The residue was purified by silica gel column chromatography (eluting with 10:1 DCM/MeOH) to afford 4-(4-nitrophenyl)pyrrolidin-2-one (1.4 g, 72% yield) as a yellow solid. LCMS (ESI+, m/z): 207 [M+H]⁺, Rt 0.667 min.

### Step 2. 1-methyl-4-(4-nitrophenyl)pyrrolidin-2-one

To a solution of 4-(4-nitrophenyl)pyrrolidin-2-one (1.4 g, 6.79 mmol, 1 eq.) in DMF (40 mL) was added sodium hydride (406 mg, 10.18 mmol, 60% wt in mineral oil) at 0° C. The mixture was stirred for 15 min followed by addition of iodomethane (2.89 g, 20.37 mmol, 3 eq). The mixture was stirred for 3 h at room temperature. The reaction was monitored by LCMS. The reaction mixture was quenched by ice/water (100 mL). The resulting mixture was extracted with EA (3×50 mL). The combined organic layers were washed with brine (2×50 mL), dried over anhydrous Na₂SO₄. After filtration, the filtrate was concentrated under reduced pressure. The residue was purified by silica gel column chromatography (eluting with 20:1 DCM/MeOH) to afford 1-methyl-4-(4-nitrophenyl)pyrrolidin-2-one (1.07 g, 72% yield) as a yellow solid. ¹H NMR (300 MHz, DMSO-d₆) δ 8.21 (d, J=9.0 Hz, 2H), 7.61 (d, J=9.0 Hz, 2H), 3.80-3.71 (m, 2H), 3.40-3.36 (m, 1H), 2.78 (s, 3H), 2.74-2.67 (m, 1H), 2.42-2.36 (m, 1H). LCMS (ESI+, m/z): 221 [M+H]⁺, Rt 0.725 min.

### Step 3. 4-(4-aminophenyl)-1-methyl-pyrrolidin-2-one

To a stirred mixture of 1-methyl-4-(4-nitrophenyl)pyrrolidin-2-one (970 mg, 4.40 mmol, 1 eq.) in EA (10 mL) and EtOH (10 mL) was added Pd/C (100 mg, 10 wt %) at room temperature under N₂ atmosphere. The resulting mixture was stirred for 3 h at room temperature under H₂ atmosphere. The reaction was monitored by LC-MS. The solids were filtered out and washed with EtOH (3×20 mL). The filtrate was concentrated under reduced pressure. The residue was purified by silica gel column chromatography (eluting with 10:1 DCM/MeOH) to afford 4-(4-aminophenyl)-1-methyl-pyrrolidin-2-one (700 mg, 84% yield) as a yellow solid. LCMS (ESI+, m/z): 191 [M+H]⁺, Rt 0.553 min.

### Step 4. Separation of Enantiomers to Obtain (R)-4-(4-aminophenyl)-1-methyl-pyrrolidin-2-one and (S)-4-(4-aminophenyl)-1-methyl-pyrrolidin-2-one (Intermediates 35 and 36)

The racemic compound 4-(4-aminophenyl)-1-methyl-pyrrolidin-2-one (700 mg, 3.66 mmol, 1 eq.) was separated by Prep-Chiral-HPLC with the following conditions (Column: CHIRAL ART Cellulose-SC, 2×25 cm, 5 μm; Mobile Phase A: Hex:DCM=3:1 (0.5% 2M NH₃-MeOH)-HPLC, Mobile Phase B: EtOH-HPLC; Flow rate: 20 mL/min; Gradient: 20% B for 11.5 min; Wave Length: 254/220 nm; RT1(min): 9.02; RT2(min): 10.82; Sample Solvent: EtOH-HPLC; Injection Volume: 0.8 mL; Number Of Runs: 7). The first eluting isomer was isolated to afford Intermediate 35 (310 mg) as a yellow solid. The second eluting isomer was isolated to afford Intermediate 36 (290 mg) as a yellow solid.

### Intermediate 27: tert-butyl (4-(4-aminophenyl)-1-oxido-1l6-thiomorpholin-1-ylidene)carbamate

### Step 1. 4-(4-nitrophenyl)thiomorpholine

To a solution of thiomorpholine (3.29 g, 31.89 mmol, 1.5 eq.) and K₂CO₃ (5.88 g, 42.52 mmol, 2 eq.) in DMF (30 mL) was added 1-fluoro-4-nitrobenzene (3.00 g, 21.26 mmol, 1 eq.) at room temperature. The mixture was stirred for 1 h at 100° C. The reaction was monitored by LC-MS. The mixture was allowed to cool down to room temperature. Then the reaction was quenched with water (100 mL) and extracted with EtOAc (3×80 mL). The combined organic layers were washed with brine (150 mL), and dried over anhydrous Na₂SO₄. After filtration, the filtrate was concentrated under reduced pressure. The residue was purified by silica gel column chromatography (eluting with 1:1 EA/PE) to afford 4-(4-nitrophenyl)thiomorpholine (4.30 g, 90% yield) as a yellow solid. LCMS (ESI+, m/z): 225 [M+H]⁺, Rt 1.093 min.

### Step 2. 4-(4-nitrophenyl)thiomorpholine 1-oxide

To a solution of 4-(4-nitrophenyl)thiomorpholine (4.30 g, 19.17 mmol, 1 eq.) in ACN (50 mL) was added H₂O₂ (19.56 mL, 632.80 mmol, 33 eq.) at room temperature. The mixture was stirred for 4 h at room temperature. The reaction was monitored by LC-MS. The resulting mixture was concentrated under vacuum and purified by silica gel column chromatography (eluting with 3:1 EA/PE) to afford 4-(4-nitrophenyl)thiomorpholine 1-oxide (3.5 g, 76% yield) as a yellow solid. LCMS (ESI+, m/z): 241 [M+H]⁺, Rt 0.678 min.

### Step 3. tert-butyl (4-(4-nitrophenyl)-1-oxido-1l6-thiomorpholin-1-ylidene)carbamate

To a stirred suspension of 4-(4-nitrophenyl)thiomorpholine 1-oxide (1.20 g, 4.99 mmol, 1 eq.), tert-butyl carbamate (878 mg, 7.49 mmol, 1.5 eq.), Rh₂(OAc)₄ (112 mg, 499 μmol, 0.1 eq.) and MgO (805 mg, 19.98 mmol, 4 eq.) in 1,2-DCE (10 mL) was added PhI(OAc)₂ (2.41 g, 7.49 mmol, 1.5 eq.) at room temperature. The resulting mixture was stirred at 70° C. for 6 h under N₂ atmosphere. The reaction was monitored by LC-MS. The reaction mixture was cooled down to room temperature and diluted with EtOAc (10 mL). The solids were filtered through a pad of celite and washed by EtOAc (10 mL×3). The filtrate was concentrated. The residue was purified by silica gel column chromatography (eluting with 2:1 EA/PE) to afford tert-butyl (4-(4-nitrophenyl)-1-oxido-1l6-thiomorpholin-1-ylidene)carbamate (750 mg, 42% yield) as a yellow solid. LCMS (ESI+, m/z): 356 [M+H]⁺, Rt 0.890 min.

### Step 4. tert-butyl (4-(4-aminophenyl)-1-oxido-1l6-thiomorpholin-1-ylidene)carbamate (Intermediate 27)

To a solution of tert-butyl (4-(4-nitrophenyl)-1-oxido-1l6-thiomorpholin-1-ylidene)carbamate (300 mg, 844 μmol, 1 eq.) in EtOH (10 mL) was added Pd/C (60 mg, 10 wt %) under N₂ atmosphere. The mixture was stirred at room temperature for 2 h under H₂ atmosphere. The reaction was monitored by LC-MS. The resulting mixture was filtered and the filter cake was washed with EtOH (3×5 mL). The filtrate was concentrated under reduced pressure and purified by silica gel column chromatography (eluting with 2:1 EA/PE) to afford tert-butyl (4-(4-aminophenyl)-1-oxido-1l6-thiomorpholin-1-ylidene)carbamate (210 mg, 76% yield) as a yellow solid. ¹H NMR (300 MHz, DMSO-d6) δ 6.86-6.77 (m, 2H), 6.55-6.51 (m, 2H), 4.71 (br, 2H), 3.67-3.51 (m, 4H), 3.47-3.36 (m, 4H), 1.40 (s, 9H). LCMS (ESI+, m/z): 326 [M+H]⁺, Rt 0.617 min.

### Intermediate 37: 4-(1-methylimino-1-oxo-1,4-thiazinan-4-yl)aniline

### Step 1. 1-imino-4-(4-nitrophenyl)-1,4-thiazinane 1-oxide

To a stirred mixture of Intermediate 27/step 3 (1.0 g, 2.81 mmol, 1 eq.) in DCM (20 mL) was added TFA (8 mL) dropwise under 0° C. The resulting mixture was stirred for 2 h at room temperature. The reaction was monitored by LC-MS. The resulting mixture was concentrated under reduced pressure. The residue was purified by silica gel column chromatography (eluting with 1:20 MeOH/DCM) to afford 1-imino-4-(4-nitrophenyl)-1,4-thiazinane 1-oxide (650 mg, 91% yield) as a brown solid. LCMS (ESI+, m/z): 255 [M+H]⁺, Rt 0.474 min.

### Step 2. 1-methylimino-4-(4-nitrophenyl)-1,4-thiazinane 1-oxide

To a stirred mixture of 1-imino-4-(4-nitrophenyl)-1,4-thiazinane 1-oxide (600 mg, 2.35 mmol, 1 eq.) in MeOH (30 mL) were added HCHO (705 mg, 23.50 mmol, 10 eq.), AcOH (0.5 mL) and NaBH₃CN (1.48 g, 23.50 mmol, 10 eq.) at 0° C. The resulting mixture was stirred for 16 h at room temperature. The reaction was monitored by LC-MS. The resulting mixture was concentrated under reduced pressure. The residue was purified by silica gel column chromatography (eluting with 1:1 EA/PE) to afford 1-methylimino-4-(4-nitrophenyl)-1,4-thiazinane 1-oxide (500 mg, 79% yield) as a brown solid. LCMS (ESI+, m/z): 269 [M+H]⁺, Rt 0.544 min.

### Step 3. 4-(1-methylimino-1-oxo-1,4-thiazinan-4-yl)aniline (Intermediate 37)

A mixture of 1-methylimino-4-(4-nitrophenyl)-1,4-thiazinane 1-oxide (500 mg, 1.86 mmol, 1 eq.) and Pd/C (100 mg, 10 wt %) in EtOH (30 mL) was stirred for 2 h at room temperature under H₂ atmosphere. The reaction was monitored by LC-MS. The resulting mixture was filtered, and the filter cake was washed with EA (3×20 ml). The filtrate was concentrated under reduced pressure. The residue was purified by silica gel column chromatography (eluting with 1:5 EA/PE) to afford 4-(1-methylimino-1-oxo-1,4-thiazinan-4-yl)aniline (380 mg, 85% yield) as a brown solid. ¹H NMR (300 MHz, DMSO-d6) δ 6.77 (d, J=9 Hz, 2H), 6.52 (d, J=9 Hz, 2H), 4.67 (br, 2H), 3.55-3.42 (m, 2H), 3.37-3.28 (m, 2H), 3.21-2.98 (m, 4H), 2.66 (s, 3H). LCMS (ESI+, m/z): 239 [M+H]⁺, Rt 0.149 min.

### Intermediate 38: 3-methyl-4-(1-methylpiperidin-4-yl)aniline

### Step 1. 1-methyl-4-(2-methyl-4-nitrophenyl)-1,2,3,6-tetrahydropyridine

A solution of 1-bromo-2-methyl-4-nitrobenzene (2.0 g, 9.26 mmol, 1 eq.), 1-methyl-4-(4,4,5,5-tetramethyl-1,3,2-dioxaborolan-2-yl)-1,2,3,6-tetrahydropyridine (2.48 g, 11.11 mmol, 1.2 eq.), Pd(dppf)Cl₂ (759 mg, 926 μmol, 0.1 eq.) and Na₂CO₃ (370 mg, 18.52 mmol, 2 eq.) in dioxane (15 mL) and H₂O (5 mL) was stirred for 1 h at 100° C. under N₂ atmosphere. The reaction was monitored by LC-MS. The mixture was allowed to cool down to room temperature followed by addition of water (50 mL). The resulting mixture was extracted with ethyl acetate (3×50 mL). The combined organic layer was washed with brine (50 mL), dried over anhydrous Na₂SO₄. After filtration, the filtrate was concentrated under reduced pressure. The residue was purified by silica gel column chromatography (eluting with 1:1 EA/PE) to afford 1-methyl-4-(2-methyl-4-nitrophenyl)-1,2,3,6-tetrahydropyridine (2.0 g, 93% yield) as a yellow solid. LCMS (ESI+, m/z): 233 [M+H]⁺, Rt 0.680 min.

### Step 2. 3-methyl-4-(1-methylpiperidin-4-yl)aniline (Intermediate 38)

To a stirred solution of 1-methyl-4-(2-methyl-4-nitrophenyl)-1,2,3,6-tetrahydropyridine (2.0 g, 8.61 mmol, 1 eq.) in MeOH (10 mL) was added Pd/C (200 mg, 10 wt %) under N₂ atmosphere. The resulting mixture was stirred for 4 h at room temperature under H₂ atmosphere. The solids were filter and washed with MeOH (2×15 mL). The filtrate was concentrated under reduced pressure. The crude product was purified by silica gel column chromatography (eluting with 5:1 DCM/MeOH) to afford 3-methyl-4-(1-methyl-4-piperidyl)aniline (0.80 g, 45% yield) as a yellow solid. ¹H NMR (300 MHz, DMSO-d6) δ 6.84 (d, J=8.7 Hz, 1H), 6.42-6.32 (m, 2H), 4.73 (br, 2H), 2.92-2.79 (m, 2H), 2.52-2.35 (m, 1H), 2.19 (s, 3H), 2.15 (s, 3H), 2.01-1.88 (m, 2H), 1.67-1.49 (m, 4H). LCMS (ESI+, m/z): 205 [M+H]⁺, Rt 0.106 min.

### Intermediate 40: 2-(4-aminophenyl)-N,N,2-trimethylpropanamide

### Step 1. N,N,2-trimethyl-2-(4-nitrophenyl)propanamide

To a stirred solution of 2-methyl-2-(4-nitrophenyl)propanoic acid (2.0 g, 9.56 mmol, 1 eq.) and N-methylmethanamine hydrochloride (779 mg, 9.56 mmol, 1 eq.) in DMF (20 mL) was added HATU (5.45 g, 14.34 mmol, 1.5 eq.) and DIEA (3.70 g, 28.68 mmol, 3 eq.) at 0° C. The resulting mixture was stirred for 2 h at room temperature. The reaction was monitored by LC-MS. The resulting mixture was diluted with EA (100 mL) and washed with brine (3×50 ml). The organic layer was dried over anhydrous Na₂SO₄. After filtration, the filtrate was concentrated under reduced pressure. The residue was purified by silica gel column chromatography (eluting with 1:2 EA/PE) to afford N,N,2-trimethyl-2-(4-nitrophenyl)propanamide (2.0 g, 88% yield) as a yellow solid. ¹H NMR (300 MHz, DMSO-d6) δ 8.24 (d, J=9.0, 2H), 7.48 (d, J=9.0, 2H), 2.82 (s, 3H), 2.46 (s, 3H), 1.49 (s, 6H). LCMS (ESI+, m/z): 237 [M+H]⁺, Rt 0.642 min.

### Step 2. 2-(4-aminophenyl)-N,N,2-trimethylpropanamide (Intermediate 40)

To a stirred solution of N,N,2-trimethyl-2-(4-nitrophenyl)propanamide (2 g, 8.47 mmol, 1 eq.) in MeOH (10 mL) was added Pd/C (200 mg, 10 wt %) under nitrogen atmosphere. The resulting mixture was stirred for 2 h at room temperature under H₂ atmosphere. The reaction was monitored by LC-MS. The solids were filtered and washed with MeOH (3×30 mL). The filtrate was concentrated under reduced pressure. The residue was purified by silica gel column chromatography (eluting with 1:1 EA/PE) to afford 2-(4-aminophenyl)-N,N,2-trimethyl-propanamide (1.5 g, 86% yield) as a yellow solid. LCMS (ESI+, m/z): 207 [M+H]⁺, Rt 0.333 min.

### Intermediates 41 and 42: (S)-3-(4-aminophenyl)-1,3-dimethylpyrrolidin-2-one and (R)-3-(4-aminophenyl)-1,3-dimethylpyrrolidin-2-one

### Step 1. 1-methyl-3-(4-nitrophenyl)pyrrolidin-2-one

To a stirred solution of 1-methylpyrrolidin-2-one (540 mg, 5.45 mmol, 1.1 eq.) in THF (10 mL) was added s-BuLi (1.3 M in cyclohexane, 4.57 mL, 1.2 eq.) dropwise at −78° C. under N₂ atmosphere. The resulting mixture was stirred for 1 h at 0° C. Then the mixture was cooled down to −78° C. To the above mixture was added ZnCl₂ (1 M in Et₂O, 5.94 mL, 1.2 eq.) dropwise. The resulting mixture was stirred for an additional 1 h at 0° C. To the above mixture was added 1-bromo-4-nitrobenzene (1.0 g, 4.95 mmol, 1 eq.), Pd₂(dba)₃ (453 mg, 495 μmol, 0.1 eq.) and DavePhos (195 mg, 495 μmol, 0.1 eq.) in THF (10 mL) dropwise. The resulting mixture was stirred for an additional 20 h at 65° C. under N₂ atmosphere. The reaction was monitored by LC-MS. The mixture was allowed to cool down to room temperature. The resulting mixture was concentrated under reduced pressure. The residue was purified by silica gel column chromatography (eluting with 10:1 DCM/MeOH). The crude product was purified by reverse phase chromatography with the following conditions (Column: C18, 40-60 nm; Mobile Phase A: water (10 mmol/L NH₄HCO₃), Mobile Phase B: ACN (20% to 30% in 10 min); 254 nm) to afford 1-methyl-3-(4-nitrophenyl)pyrrolidin-2-one (300 mg, 28% yield) as a yellow solid. LCMS (ES, m/z): 221 [M+H]⁺, Rt 0.619 min.

### Step 2. 1,3-dimethyl-3-(4-nitrophenyl)pyrrolidin-2-one

To a stirred solution of 1-methyl-3-(4-nitrophenyl)pyrrolidin-2-one (1.0 g, 4.54 mmol, 1 eq.) in DMF (10 mL) was added NaH (363 mg, 9.08 mmol, 60% wt in mineral oil, 2 eq.) in portions at 0° C. under N₂ atmosphere. The resulting mixture was stirred for 1 h at room temperature. To the above solution was added Mel (967 mg, 6.81 mmol, 1.5 eq.) dropwise. The resulting mixture was stirred for an additional 2 h at room temperature. The reaction was monitored by LC-MS. The mixture was quenched with water/ice (30 mL). The resulting mixture was extracted with EA (3×30 mL). The combined organic layers were dried over anhydrous Na₂SO₄. After filtration, the filtrate was concentrated under reduced pressure. The residue was purified by silica gel column chromatography (eluting with 1:1 EA/PE) to afford 1,3-dimethyl-3-(4-nitrophenyl)pyrrolidin-2-one (750 mg, 71% yield) as a yellow solid. LCMS (ESI+, m/z): 235 [M+H]⁺, Rt 0.659 min.

### Step 3. 3-(4-aminophenyl)-1,3-dimethylpyrrolidin-2-one

A mixture of 1,3-dimethyl-3-(4-nitrophenyl)pyrrolidin-2-one (750 mg, 3.20 mmol) and Pd/C (150 mg, 10 wt %) in MeOH (10 mL) was stirred for 2 h at room temperature under H₂ atmosphere. The reaction was monitored by TLC. After filtration, the filtrate was concentrated under reduced pressure to afford 3-(4-aminophenyl)-1,3-dimethylpyrrolidin-2-one (550 mg, 85% yield) as a white solid. ¹H NMR (300 MHz, DMSO-d6) δ 6.99 (d, J=9 Hz, 2H), 6.50 (d, J=9 Hz, 2H), 4.92 (br, 2H), 3.26-3.15 (m, 2H), 2.77 (s, 3H), 2.28-2.21 (m, 1H), 2.05-1.96 (m, 1H), 1.31 (s, 3H). LCMS (ESI+, m/z): 205 [M+H]⁺, Rt 0.440 min.

### Step 4. Separation of enantiomers to obtain (S)-3-(4-aminophenyl)-1,3-dimethylpyrrolidin-2-one and (R)-3-(4-aminophenyl)-1,3-dimethylpyrrolidin-2-one (Intermediates 41 and 42)

The racemic compound 3-(4-aminophenyl)-1,3-dimethylpyrrolidin-2-one (550 mg, 2.71 mmol, 1 eq.) was separated by Prep-Chiral-HPLC with the following conditions (Column: (R, R)-WHELK-O1-Kromasil, 2.12×25 cm, 5 μm; Mobile Phase A: Hex (0.5% 2 M NH₃-MeOH)-HPLC, Mobile Phase B: EtOH-HPLC; Flow rate: 20 mL/min; Gradient: 40% B for 16 min; Wave Length: 220/254 nm; RT1(min): 11.76; RT2(min): 14.1) to afford Intermediate 41 (240 mg) as a yellow solid and Intermediate 42 (220 mg) as a yellow solid.

### Intermediate 43: 6-(tert-butyl)pyridin-3-amine

### Step 1. N-(6-(tert-butyl)pyridin-3-yl)-1,1-diphenylmethanimine

A mixture of 5-bromo-2-tert-butyl-pyridine (600 mg, 2.80 mmol, 1 eq.), diphenylmethanimine (609 mg, 3.36 mmol, 1.2 eq.), Cs₂CO₃ (2.74 g, 8.41 mmol, 3 eq.), BrettPhos-G3 (254 mg, 280 μmol, 0.1 eq.) and BrettPhos (150 mg, 280 μmol, 0.1 eq.) in 1,4-dioxane (20 mL) was stirred for 1 h at 100° C. under N₂ atmosphere. The reaction was monitored by TLC. The mixture was allowed to cool down to room temperature. The solids were filtered out. The filter cake was washed with DCM (3×10 mL). The filtrate was concentrated under reduced pressure. The residue was purified by silica gel column chromatography (eluting with 1:1 EA/PE) to afford N-(6-tert-butyl-3-pyridyl)-1,1-diphenyl-methanimine (700 mg, 80% yield) as a yellow oil. LCMS (ESI+, m/z): 315 [M+H]⁺, Rt 0.839 min.

### Step 2. 6-(tert-butyl)pyridin-3-amine (Intermediate 43)

To a stirred solution of N-(6-tert-butyl-3-pyridyl)-1,1-diphenyl-methanimine (690 mg, 2.19 mmol) in THF (9 mL) was added AcOH (3 mL) and H₂O (1.5 mL). The resulting mixture was stirred for 1 h at 100° C. The reaction was monitored by LC-MS. The mixture was allowed to cool down to room temperature and concentrated under vacuum. The crude product was purified by reverse phase chromatography (Column: C18; Mobile phase, A: water (containing 10 mmol/L NH₄HCO₃) and B: ACN (5% to 100% B over 40 min); Detector, UV 254 nm) to afford 6-tert-butylpyridin-3-amine (220 mg, 67% yield) as a yellow oil. ¹H NMR (300 MHz, DMSO-d6) δ 7.87 (s, 1H), 7.07-7.04 (m, 1H), 6.88-6.84 (m, 1H), 5.02 (br, 2H), 1.23 (s, 9H). LCMS (ESI+, m/z): 151 [M+H]⁺, Rt 0.506 min.

### Intermediate 45: 4-(3-oxa-8-azabicyclo[3.2.1]octan-8-yl)-3-methylaniline

### Step 1. 8-(2-methyl-4-nitrophenyl)-3-oxa-8-azabicyclo[3.2.1]octane

A mixture of 1-bromo-2-methyl-4-nitrobenzene (1.5 g, 6.94 mmol, 1 eq.), 3-oxa-8-azabicyclo[3.2.1]octane hydrochloride salt (1.56 g, 10.42 mmol, 1.5 eq.), Cs₂CO₃ (9.05 g, 27.77 mmol, 4 eq.), RuPhos Pd G3 (581 mg, 694 μmol, 0.1 eq.) and RuPhos (648 mg, 1.39 mmol, 0.2 eq.) in 1,4-dioxane (15 mL) was stirred for 2 h at 100° C. under N₂ atmosphere. The reaction was monitored by LC-MS. The resulting mixture was allowed to cool down to room temperature. After filtration, the filtrate was concentrated under reduced pressure. The residue was purified by silica gel column chromatography (eluting with 1:9 EA/PE) to afford 8-(2-methyl-4-nitrophenyl)-3-oxa-8-azabicyclo[3.2.1]octane (1.5 g, 87% yield) as a white solid. LCMS (ESI+, m/z): 249 [M+H]⁺, Rt 0.790 min.

### Step 2. 4-(3-oxa-8-azabicyclo[3.2.1]octan-8-yl)-3-methylaniline (Intermediate 45)

A mixture of (8-(2-methyl-4-nitrophenyl)-3-oxa-8-azabicyclo[3.2.1]octane (580 mg, 2.34 mmol, 1 eq.) and Pd/C (120 mg, 10 wt %) in EA (15 mL) and EtOH (15 mL) was stirred for 2 h at room temperature under H₂ atmosphere. The reaction was monitored by LC-MS. After filtration, the filtrate was concentrated under reduced pressure. The residue was purified by silica gel column chromatography (eluting with 1:3 EA/PE) to afford 3-methyl-4-[(1S,5R)-3-oxa-8-azabicyclo[3.2.1]octan-8-yl]aniline (450 mg, 88% yield) as a white solid. ¹H NMR (300 MHz, DMSO-d6) δ 6.54 (d, J=9 Hz, 1H), 6.42 (s, 1H), 6.30 (d, J=9 Hz, 1H), 4.60 (br, 2H), 3.72 (d, J=12 Hz, 2H), 3.56-3.52 (m, 2H), 3.36-3.34 (m, 2H), 2.19 (s, 3H), 1.90-1.79 (m, 4H). LCMS (ESI+, m/z): 219 [M+H]⁺, Rt 0.499 min.

### Intermediate 48: 7-amino-2,4,4-trimethyl-3H-isoquinolin-1-one

### Step 1. 2-bromo-N-methyl-5-nitrobenzamide

To a stirred solution of 2-bromo-5-nitrobenzoic acid (6.00 g, 24.4 mmol, 1 eq.) and MeNH₂·HCl (2.47 g, 36.58 mmol, 1.5 eq.) in DMF (100 mL) was added PyBOP (19.04 g, 36.58 mmol, 1.5 eq.) and DIPEA (12.61 g, 97.56 mmol, 4 eq.).

The resulting mixture was stirred for 2 h at room temperature. The reaction was monitored by LC-MS. The resulting mixture was quenched by cool water (300 mL) and extracted with EA (3×200 mL). The combined organic layers were washed with brine (2×200 mL), dried over anhydrous Na₂SO₄. After filtration, the filtrate was concentrated under reduced pressure. The residue was purified by silica gel column chromatography (eluting with 1:1 EA/PE) to afford 2-bromo-N-methyl-5-nitrobenzamide (3.50 g, 55% yield) as a yellow solid. LCMS (ESI+, m/z): 259, 261 [M+H]⁺, Rt 0.637 min.

### Step 2. 2-bromo-N-methyl-N-(2-methylallyl)-5-nitrobenzamide

To a solution of 2-bromo-N-methyl-5-nitrobenzamide (3.50 g, 13.5 mmol, 1 eq.) in DMF (50 mL) was added NaH (60%, 1.04 g, 27.02 mmol, 2 eq.) at 0° C. The mixture was stirred for 0.5 h at room temperature. Then 3-bromo-2-methylprop-1-ene (2.37 g, 17.56 mmol, 1.3 eq.) was added at 0° C. The mixture was allowed to warm to room temperature and stirred for 1 h. The reaction was monitored by LC-MS. The reaction mixture was quenched by ice/water (150 mL) and extracted with EtOAc (3×200 mL). The combined organic layers were washed with brine (200 mL), dried over anhydrous Na₂SO₄. After filtration, the filtrate was concentrated under reduced pressure. The residue was purified by silica gel column chromatography (eluting with 1:1 EA/PE) to afford 2-bromo-N-methyl-N-(2-methylallyl)-5-nitrobenzamide (2.9 g, 69% yield) as a yellow solid. LCMS (ESI+, m/z): 313, 315 [M+H]⁺, Rt 0.898 min.

### Step 3. 2,4,4-trimethyl-7-nitro-3,4-dihydroisoquinolin-1(2H)-one

A mixture of 2-bromo-N-methyl-N-(2-methylallyl)-5-nitrobenzamide (2.90 g, 9.26 mmol, 1 eq.), TEACl (1.68 g, 9.26 mmol, 1 eq.), HCOONa (693 mg, 10.19 mmol, 1.1 eq.), NaOAc (1.67 g, 20.37 mmol, 2.2 eq.) and Pd(OAc)₂, (208 mg, 926 μmol. 0.1 eq.) in DMF (40 mL) was stirred at 70° C. for 16 h under N₂ atmosphere. The reaction was monitored by LC-MS. The reaction was cooled to room temperature and filtered through a pad of celite. The filter cake was washed with DCM (3×50 mL). The filtrate was concentrated under reduced pressure and the resulting residue purified by silica gel column chromatography (eluting with 1:4 EA/PE) to afford 2,4,4-trimethyl-7-nitro-3,4-dihydroisoquinolin-1(2H)-one (1.6 g, 74% yield) as a yellow solid. LCMS (ESI+, m/z): 235 [M+H]⁺, Rt 0.780 min.

### Step 4. 7-amino-2,4,4-trimethyl-3H-isoquinolin-1-one (Intermediate 48)

To a stirred solution of 2,4,4-trimethyl-7-nitro-3H-isoquinolin-1-one (500 mg, 2.13 mmol, 1 eq.) in EtOH (10 mL) was added Pd/C (100 mg, 10 wt %) under N₂ atmosphere. The resulting mixture was stirred for 16 h at room temperature under H2 atmosphere. The solids were filtered out and washed with EtOH (2×5 mL). The filtrate was concentrated under reduced pressure. The crude product was purified by silica gel column chromatography (eluting with 10:1 DCM/MeOH) to afford 7-amino-2,4,4-trimethyl-3H-isoquinolin-1-one (380 mg, 1.77 mmol, 83% yield) as a yellow solid. LCMS (ES, m/z): 205 [M+H]⁺, Rt 0.294 min.

### Intermediates 49 and 50: (S)-5-(5-aminopyridin-2-yl)-1-methylpyrrolidin-2-one and (R)-5-(5-aminopyridin-2-yl)-1-methylpyrrolidin-2-one

### Step 1. methyl 4-(5-bromopyridin-2-yl)-4-oxobutanoate

A solution of 5-bromopyridine-2-carbaldehyde (10 g, 53.8 mmol, 1 eq.), 2-(3-ethyl-4-methyl-thiazol-3-ium-5-yl)ethanol bromide (2.71 g, 10.8 mmol, 0.2 eq.), Et₃N (10.9 g, 107.6 mmol, 2 eq.) and methyl prop-2-enoate (5.55 g, 64.5 mmol, 1.2 eq.) in MeOH (300 mL) was stirred for 16 h at 70° C. The reaction was monitored by LC-MS. The mixture was allowed to cool down to room temperature. The resulting mixture was concentrated under reduced pressure. The residue was diluted with EA (300 mL) and washed with brine (3×100 mL). The combined organic layers were dried over anhydrous Na₂SO₄. After filtration, the filtrate was concentrated under reduced pressure. The residue was purified by silica gel column chromatography (eluting with 1:4 EA/PE) to afford methyl 4-(5-bromopyridin-2-yl)-4-oxobutanoate (4.0 g, 31% yield) as a yellow solid. LCMS (ESI+, m/z): 237 [M+H]⁺, Rt 0.642 min.

### Step 2. 5-(5-bromopyridin-2-yl)pyrrolidin-2-one

A solution of methyl 4-(5-bromopyridin-2-yl)-4-oxobutanoate (4.0 g, 14.70 mmol, 1 eq.) and NH₄OAc (11.32 g, 147.0 mmol, 10 eq.) in MeOH (70 mL) was stirred for 1 h at room temperature. To the above mixture was added NaBH₃CN (1.02 g, 16.17 mmol, 1.1 eq.) in portions. The resulting mixture was stirred for an additional 4 h at 70° C. under N₂ atmosphere. The reaction was monitored by LC-MS. The mixture was allowed to cool down to room temperature. The resulting mixture was concentrated under reduced pressure. The residue was diluted with EA (200 mL) and washed with brine (3×30 mL). The combined organic layers were dried over anhydrous Na₂SO₄. After filtration, the filtrate was concentrated under reduced pressure. The residue was purified by silica gel column chromatography (eluting with 24:1 DCM/MeOH) to afford 5-(5-bromopyridin-2-yl)pyrrolidin-2-one (2.2 g, 62% yield) as a yellow solid. LCMS (ESI+, m/z): 241, 243 [M+H]⁺, Rt 0.547 min.

### Step 3. 5-(5-bromopyridin-2-yl)-1-methylpyrrolidin-2-one

To a solution of 5-(5-bromopyridin-2-yl)pyrrolidin-2-one (2.1 g, 8.71 mmol, 1 eq.) in DMF (30 mL) was added NaH (522 mg, 13.07 mmol, 60% suspension in mineral oil, 1.5 eq.) in portions at 0° C. The mixture was stirred for 0.5 h at room temperature under N₂ atmosphere followed by dropwise addition of Mel (1.48 g, 10.45 mmol, 1.2 eq.) at 0° C. The mixture was stirred for an additional 1.5 h at room temperature under N₂ atmosphere. The reaction was monitored by TLC. The resulting mixture was quenched with ice/water (100 mL) and extracted with EA (3×60 mL). The combined organic phase was dried over anhydrous Na₂SO₄. After filtration, the filtrate was concentrated under reduced pressure. The residue was purified by silica gel column chromatography (eluting with 19:1 DCM/MeOH) to afford 5-(5-bromopyridin-2-yl)-1-methylpyrrolidin-2-one (2.0 g, 90% yield) as yellow oil. LCMS (ESI+, m/z): 255, 257 [M+H]⁺, Rt 0.542 min.

### Step 4. 5-(5-((diphenylmethylene)amino)pyridin-2-yl)-1-methylpyrrolidin-2-one

A mixture of 5-(5-bromopyridin-2-yl)-1-methylpyrrolidin-2-one (1.9 g, 7.45 mmol, 1 eq.), diphenylmethanimine (1.62 g, 8.94 mmol, 1.2 eq.), Pd₂(dba)₃ (694 mg, 745 μmol, 0.1 eq.), BINAP (463 mg, 745 μmol, 0.1 eq.) and Cs₂CO₃ (4.86 g, 14.90 mmol, 2 eq.) in toluene (20 mL) was stirred for 2 h at 100° C. under N₂ atmosphere. The reaction was monitored by LC-MS. The mixture was allowed to cool down to room temperature. The resulting mixture was diluted with ethyl acetate (80 mL) and washed with brine (3×20 mL). The combined organic layers were dried over anhydrous Na₂SO₄. After filtration, the filtrate was concentrated under reduced pressure. The residue was purified by silica gel column chromatography (eluting with 2:1 EA/PE) to afford 5-(5-((diphenylmethylene)amino)pyridin-2-yl)-1-methylpyrrolidin-2-one (2.0 g, 76% yield) as a yellow solid. LCMS (ESI+, m/z): 356 [M+H]⁺, Rt 0.720 min.

### Step 5. 5-(5-Aminopyridin-2-yl)-1-methylpyrrolidin-2-one

A solution of 5-(5-((diphenylmethylene)amino)pyridin-2-yl)-1-methylpyrrolidin-2-one (2.0 g, 5.63 mmol, 1 eq.) in THF (12 mL), H₂O (2 mL) and AcOH (4 mL) was stirred for 1 h at 100° C. The reaction was monitored by LC-MS. The mixture was allowed to cool down to room temperature. The resulting mixture was concentrated under reduced pressure. The crude product was purified by reverse phase chromatography (Column: C18; Mobile phase, A: water (containing 10 mmol/L NH₄HCO₃) and B: ACN (5% to 10% over 15 min); Detector, UV 254 nm, 280 nm) to afford 5-(5-aminopyridin-2-yl)-1-methylpyrrolidin-2-one. (900 mg, 84% yield). ¹H NMR (300 MHz, MeOD-d4) δ 8.01-8.00 (m, 1H), 7.13-7.07 (m, 2H), 4.64-4.60 (m, 1H), 2.63 (s, 3H), 2.60-2.54 (m, 1H), 2.51-2.40 (m, 2H), 2.07-1.96 (m, 1H). LCMS (ESI+, m/z): 192 [M+H]⁺, Rt 0.418 min.

### Step 6. Separation of Enantiomers to Obtain (S)-5-(5-aminopyridin-2-yl)-1-methylpyrrolidin-2-one and (R)-5-(5-aminopyridin-2-yl)-1-methylpyrrolidin-2-one (Intermediates 49 and 50)

The racemic product 5-(5-aminopyridin-2-yl)-1-methylpyrrolidin-2-one (900 mg, 4.71 mmol) was separated by prep-Chiral-HPLC with the following conditions (Column: CHIRALPAK IH, 3×25 cm, 5 μm; Mobile Phase A: CO₂, Mobile Phase B: IPA (0.5% 2M NH₃-MeOH); Flow rate: 70 mL/min; Gradient: isocratic 50% B; Column Temperature: 35° C.; Back Pressure: 100 bar; Wave Length: 220 nm; RT1(min): 4.51; RT2(min): 6.39) to afford Intermediate 49 (400 mg, 44% yield) as a yellow solid and Intermediate 50 (400 mg, 44% yield) as a yellow solid. LCMS (ESI+, m/z): 192 [M+H]⁺, Rt 0.418 min.

### Intermediate 51: tert-butyl 6-(4-aminophenyl)-2,6-diazaspiro[3.3]heptane-2-carboxylate

### Step 1. tert-butyl 6-(4-nitrophenyl)-2,6-diazaspiro[3.3]heptane-2-carboxylate

To a stirred mixture of 1-fluoro-4-nitrobenzene (5 g, 35.44 mmol, 1.0 eq.) and tert-butyl 2,6-diazaspiro[3.3]heptane-2-carboxylate (8.43 g, 42.52 mmol, 1.2 eq.) in DMF (30 mL) was added K₂CO₃ (14.69 g, 106.31 mmol, 3.0 eq.). The resulting mixture was stirred for 5 h at 80° C. The reaction was monitored by LC-MS. The mixture was allowed to cool down to room temperature. The resulting mixture was diluted with H₂O (200 mL). The resulting mixture was extracted with EA (3×100 mL). The combined organic layers were washed with brine (2×200 mL), dried over anhydrous Na₂SO₄. After filtration, the filtrate was concentrated under reduced pressure. The residue was purified by silica gel column chromatography (eluting with 1:2 EA/PE) to afford tert-butyl 6-(4-nitrophenyl)-2,6-diazaspiro[3.3]heptane-2-carboxylate (10.5 g, 31.28 mmol, 92% yield) as a yellow solid. ¹H NMR (300 MHz, DMSO-d6) δ 8.05 (d, J=9.0 Hz, 2H), 6.44 (d, J=9.3 Hz, 2H), 4.17 (s, 4H), 4.06 (s, 4H), 1.39 (s, 9H). LCMS (ES, m/z): 320 [M+H]⁺, Rt 0.675 min.

### Step 2. tert-butyl 6-(4-aminophenyl)-2,6-diazaspiro[3.3]heptane-2-carboxylate (Intermediate 51)

To a stirred solution of tert-butyl-6-(4-nitrophenyl)-2,6-diazaspiro[3.3]heptane-2-carboxylate (10.5 g, 31.28 mmol, 1 eq.) in EtOH (500 mL) was added Pd/C (1 g, 10%) at room temperature under N₂ atmosphere. The resulting mixture was stirred for 1 h at room temperature under the H₂ atmosphere. The solids were filtered and washed with EtOH (3×50 mL). The filtrate was concentrated under reduced pressure to afford tert-butyl 6-(4-amino phenyl)-2,6-diazaspiro[3.3]heptane-2-carboxylate (7.5 g, crude) as a red solid. LCMS (ES, m/z): 290[M+H]⁺, Rt 0.477 min.

### Intermediate 52. (5S)-3-(6-bromo-2-pyridyl)-5-methyl-oxazolidin-2-one

A mixture of (5S)-5-methyloxazolidin-2-one (840.00 mg, 8.31 mmol, 1.2 eq.), 3,4,7,8-tetramethyl-1,10-phenanthroline (490.83 mg, 2.08 mmol, 0.3 eq.), tripotassium phosphate (4.41 g, 20.77 mmol, 3.0 eq.), Copper(I) Iodide (263.72 mg, 1.38 mmol, 0.2 eq.) and 2,6-dibromopyridine (1.64 g, 6.92 mmol, 1.0 eq.) in toluene (15 mL) was irradiated with microwave for 0.5 h at 120° C. under a N₂ atmosphere. The mixture was allowed to cool down to room temperature. The desired product could be detected by LC-MS. The solids were filtrated and washed with EA (3×15 mL). The filtrate was concentrated under reduced pressure. The residue was purified by silica gel column chromatography (eluting with 1:2 EA/PE) to afford (5S)-3-(6-bromo-2-pyridyl)-5-methyl-oxazolidin-2-one (1.18 g, 4.59 mmol, 66% yield) as a white solid.

¹H NMR (300 MHz, CDCl₃) δ 8.11 (d, J=8.1 Hz, 1H), 7.54 (d, J=7.8 Hz, 1H), 7.19 (d, J=7.8 Hz, 1H), 4.95-4.85 (m, 1H), 4.52 (d, J=8.4 Hz, 1H), 4.09-4.04 (m, 1H), 1.48 (d, J=6.3 Hz, 3H). LCMS (ES, m/z): 257, 529 [M+H]⁺. Rt 0.715 min.

TABLE 7
The following compounds were prepared in an analogous manner as
Intermediate 52.

| Structure/Intermediate # | LCMS |
|---|---|
| 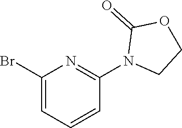 [SMILES: O=C1OCCN1c1cccc(Br)n1] | LCMS (ES, m/z): 243, 245 [M + H]⁺, Rt 0.703 min. |
| Intermediate 158 | |
| 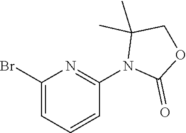 | LCMS (ES, m/z): 271, 273 [M + H]⁺, Rt 0.747 min. |
| Intermediate 162 | |
| 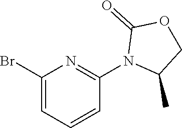 | LCMS (ES, m/z): 257, 259 [M + H]⁺, Rt 0.750 min. |
| Intermediate 165 | |
| 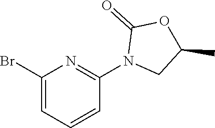 | LCMS (ES, m/z): 257, 259 [M + H]⁺, Rt 0.928 min. |
| Intermediate 166 | |

### Intermediate 53. (6-bromo-2-pyridyl)imino-dimethyl-oxo-sulfane

To a stirred mixture of 2,6-dibromopyridine (1 g, 4.22 mmol, 1 eq.) in 1,4-dioxane (35 mL) was added imino-dimethyl-oxo-sulfane (393 mg, 4.22 mmol, 1 eq.), (5-diphenylphosphanyl-9,9-dimethyl-xanthen-4-yl)-diphenyl-phosphane (244 mg, 422.13 mol), XantPhos Pd G2 (365 mg, 422.13 μmol, 0.1 eq.) and sodium 2-methylpropan-2-olate (811 mg, 8.44 mmol, 2 eq.). The resulting mixture was stirred for 2 h at 80° C. under the N₂ atmosphere. The reaction was monitored by LC-MS. The solids were filtered out and washed with EA (3×10 mL). The filtration was concentrated under reduced pressure. The residue was purified by silica gel column chromatography (eluting with 3:1 PE/EA) to afford (6-bromo-2-pyridyl)imino-dimethyl-oxo-sulfane (550 mg, 2.21 mmol, 52% yield) as a yellow solid. LCMS (ES, m/z): 249, 251 [M+H]⁺, Rt 0.589 min.

### Intermediate 56. 3-methyl-4-[(1R,4R)-5-methyl-2,5-diazabicyclo[2.2.1]heptan-2-yl]aniline

The titled compound was prepared in a similar manner as Intermediate 45, except with (1R,4R)-2-methyl-2,5-diazabicyclo[2.2.1]heptane dihydrobromide in place of 3-oxa-8-azabicyclo[3.2.1]octane hydrochloride salt. LCMS (ES, m/z): 218 [M+H]⁺

### Intermediate 57. 3-methyl-4-[(1S,4S)-5-methyl-2,5-diazabicyclo[2.2.1]heptan-2-yl]aniline

The titled compound was prepared in a similar manner as Intermediate 45, except with (1S,4S)-2-methyl-2,5-diazabicyclo[2.2.1]heptane dihydrobromide in place of 3-oxa-8-azabicyclo[3.2.1]octane hydrochloride salt. LCMS (ES, m/z): 218 [M+H]⁺

### Intermediate 70. tert-butyl 4-((5-aminopyridin-2-yl)oxy)piperidine-1-carboxylate

### Step 1. tert-butyl 4-((5-nitropyridin-2-yl)oxy)piperidine-1-carboxylate

To a stirred solution of tert-butyl 4-hydroxypiperidine-1-carboxylate (1.2 g, 5.96 mmol, 1.0 eq.) in THF (15 mL) was added NaH (60% in oil, 715.6 mg, 17.89 mmol, 3.0 eq.) in portions at 0° C. under N₂ atmosphere. The reaction mixture was stirred at 0° C. for 0.5 h. Then a solution of 2-chloro-5-nitro-pyridine (1.89 g, 11.92 mmol, 2.0 eq.) in THF (10 mL) was added dropwise and the mixture was stirred for 12 h at room temperature. The reaction was monitored by LCMS. Then saturated NH₄Cl (80 mL) was added. The mixture was extracted with EA (3×50 mL). The combined organic layers were washed with brine (80 mL), dried over anhydrous Na₂SO₄. After filtration, the filtrate was concentrated under reduced pressure. The residue was purified by silica gel column chromatography (eluting with 1:1 EA/PE) to afford tert-butyl 4-[(5-nitro-2-pyridyl) oxy]piperidine-1-carboxylate (1.5 g, 4.64 mmol, 78% yield) as a yellow oil. LCMS (ES, m/z): 268 [M+H-56]⁺, Rt 0.794 min.

### Step 2. tert-butyl 4-((5-aminopyridin-2-yl)oxy)piperidine-1-carboxylate (Intermediate 70)

To a stirred solution of tert-butyl 4-[(5-nitro-2-pyridyl)oxy]piperidine-1-carboxylate (1.5 g, 4.64 mmol, 1.0 eq.) in EtOH (20 mL) and EA (20 mL) was added Pd/C (300 mg, 10 wt %) in portions at room temperature under N₂ atmosphere. The resulting mixture was stirred for 2 h at room temperature under H₂ atmosphere. The reaction was monitored by LCMS. The solids were filtered out and washed with EtOH (3×10 mL). The filtrate was concentrated and purified by silica gel column chromatography (eluting with 1:10 MeOH/DCM) to afford tert-butyl 4-[(5-amino-2-pyridyl)oxy]piperidine-1-carboxylate (1 g, 3.41 mmol, 73% yield) as a yellow solid. LCMS (ES, m/z): 294 [M+H]⁺, Rt 0.516 min.

TABLE 8
The following intermediates were prepared in an analogous manner as
Intermediate 70.

| Structure/Intermediate # | LCMS |
|---|---|
| 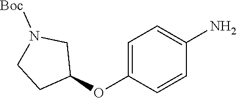 [SMILES: CC(C)(C)OC(=O)N1CC[C@H](Oc2ccc(N)cc2)C1] | LCMS (ES, m/z): 279 [M + H]+, Rt 0.580 min |
| Intermediate 71 | |
| 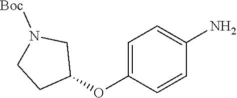 [SMILES: CC(C)(C)OC(=O)N1CC[C@@H](Oc2ccc(N)cc2)C1] | LCMS (ES, m/z): 279 [M + H]+, Rt 0.574 min. |
| Intermediate 72 | |
| 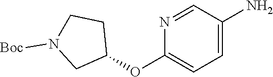 [SMILES: CC(C)(C)OC(=O)N1CC[C@H](Oc2ccc(N)cn2)C1] | LCMS (ES, m/z): 280 [M + H]+, Rt 0.704 min. |
| Intermediate 75 | |
| 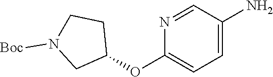 [SMILES: CC(C)(C)OC(=O)N1CC[C@H](Oc2ccc(N)cn2)C1] | LCMS (ES, m/z): 280 [M + H]+, Rt 0.661 min. |
| Intermediate 76 | |
| 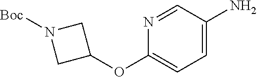 [SMILES: CC(C)(C)OC(=O)N1CC(Oc2ccc(N)cn2)C1] | LCMS (ES, m/z): 266 [M + H]+, Rt 0.576 min. |
| Intermediate 77 | |

### Intermediate 73. tert-Butyl (R)-3-(4-amino-2-methylphenoxy)pyrrolidine-1-carboxylate

### Step 1. tert-butyl (R)-3-(2-methyl-4-nitrophenoxy)pyrrolidine-1-carboxylate (Compound 2)

To a stirred solution of 2-methyl-4-nitro-phenol (1.2 g, 7.84 mmol, 1.0 eq.), tert-butyl (3S)-3-hydroxypyrrolidine-1-carboxylate (1.76 g, 9.40 mmol, 1.2 eq.) and PPh₃ (4.11 g, 15.67 mmol, 2.0 eq.) in THF (15 mL) was added DIAD (3.17 g, 15.67 mmol, 2.0 eq.) dropwise. The resulting mixture was stirred for 12 h at room temperature under N₂ atmosphere. The reaction was monitored by LCMS. Then saturated NH₄Cl (50 mL) was added. The mixture was extracted with EA (3×50 mL), the combined organic layers were washed with brine (2×60 mL), dried over anhydrous Na₂SO₄. After filtration, the filtrate was concentrated under reduced pressure. The residue was purified by silica gel column chromatography (eluting with 1:1 EA/PE) to afford tert-butyl (3R)-3-(2-methyl-4-nitro-phenoxy)pyrrolidine-1-carboxylate (2.07 g, 5.78 mmol, 82% yield) as a yellow solid. LCMS (ES, m/z): 267 [M+H-56]⁺, Rt 0.805 min.

### Step 2. tert-butyl (R)-3-(4-amino-2-methylphenoxy)pyrrolidine-1-carboxylate

To a stirred solution of tert-butyl (3R)-3-(2-methyl-4-nitro-phenoxy)pyrrolidine-1-carboxylate (1.2 g, 3.72 mmol, 1.0 eq.) in EtOH (25 mL) and EA (25 mL) was added Pd/C (250 mg, 10 wt %) in portions under N₂ atmosphere. The resulting mixture was stirred for 2 h at room temperature under the H₂ atmosphere. The reaction was monitored by LCMS. The solids were filtered out and washed with EtOH (3×10 mL). The filtrate was concentrated and purified by silica gel column chromatography (eluting with 1:10 MeOH/DCM) to afford tert-butyl (3R)-3-(4-amino-2-methyl-phenoxy)pyrrolidine-1-carboxylate (585 mg, 1.80 mmol, 54% yield,) as a yellow solid. LCMS (ES, m/z): 237 [M+H-56]⁺, Rt 0.685 min.

¹H NMR (300 MHz, DMSO-d6) δ 6.66 (d, J=8.4 Hz, 1H), 6.43-6.29 (m, 2H), 4.73-4.70 (m, 1H), 4.59 (br, 2H), 3.47-3.34 (m, 4H), 2.08-1.91 (m, 5H), 1.40 (d, J=6.3 Hz, 9H).

### Intermediate 74. tert-butyl (S)-3-(4-amino-2-methylphenoxy)pyrrolidine-1-carboxylate

Prepared in an analogous manner as Intermediate 73. LCMS (ES, m/z): 293 [M+H]⁺, Rt 0.686 min.

### Intermediate 86. 2-bromo-5-methylpyrrolo[2,3-b]pyrazine

To a solution of 2-bromo-5H-pyrrolo[2,3-b]pyrazine (200 mg, 1.01 mmol, 1 eq.) in DMF (5 mL) was added NaH (60% in oil, 101 mg, 2.53 mmol, 2.5 eq.) in portions at 0° C. The mixture was stirred for 0.5 h at 0° C. under the N₂ atmosphere. Then Mel (172.03 mg, 1.212 mmol, 1.2 eq.) was added dropwise and the mixture was allowed to warm to room temperature and stirred for 2 h. The reaction was monitored with LCMS. The mixture was quenched by water (20 mL) and extracted with DCM (3×20 mL). The combined organic layer was dried over anhydrous Na₂SO₄. After filtration, the filtrate was concentrated under reduced pressure. The residue was purified by silica gel column chromatography (eluting with 1:1 EA/PE) to afford 2-bromo-5-methylpyrrolo[2,3-b]pyrazine (150 mg, 70% yield) as a yellow solid. LCMS (ES, m/z): 212, 214 [M+H]⁺. Rt 0.653 min.

### Intermediate 95 and Intermediate 96. (S)-4-(1,3-dimethylpyrrolidin-3-yl)aniline and (R)-4-(1,3-dimethylpyrrolidin-3-yl)aniline

### Step 1. 3-(4-Bromophenyl)-1,3-dimethyl-pyrrolidin-2-one

To a stirred solution of 3-(4-bromophenyl)pyrrolidin-2-one (2 g, 8.33 mmol, 1 eq.) in DMF (20 mL) was added NaH (833.00 mg, 20.82 mmol, 60% purity, 2.5 eq.) at 0° C. under nitrogen atmosphere. The resulting mixture was stirred for 1 h at 0° C. To the above mixture was added iodomethane (1.56 mL, 24.99 mmol, 3 eq.) dropwise at 0° C. The resulting mixture was stirred for additional 2 h at room temperature. The reaction was monitored by LC-MS. The resulting mixture was quenched with water/ice (50 mL), and extracted with EA (3×20 mL). The combined organic layers were dried over Na₂SO₄. After filtration, the filtrate was concentrated under reduced pressure. The residue was purified by silica gel column chromatography (eluting with 3:1 EA/PE) to afford the crude product, and then purified by reverse phase chromatography with the following conditions (Column: C18 80 g, 40-60 nm; Mobile Phase A: water (10 mmol/L NH₄HCO₃), Mobile Phase B: CH₃CN (40% to 60% in 15 min); 254 nm) to afford 3-(4-bromophenyl)-1,3-dimethyl-pyrrolidin-2-one (1.4 g, 4.96 mmol, 60% yield) as a white solid. LCMS (ES, m/z): 268, 270 [M+H]⁺, Rt 0.727 min.

### Step 2. 3-(4-bromophenyl)-1,3-dimethyl-pyrrolidine

To a solution of 3-(4-bromophenyl)-1,3-dimethyl-pyrrolidin-2-one (1.2 g, 4.48 mmol, 1 eq.) and BF₃-Et₂O (2.86 g, 20.14 mmol, 4.5 eq.) in THF (10 mL) was added sodium borohydride (507.91 mg, 13.43 mmol, 3 eq.) at −10° C. The resulting mixture was stirred for 16 h at 20° C. under nitrogen atmosphere. The reaction was monitored by LCMS. The resulting mixture was concentrated under reduced pressure. To the above mixture was added 10 mL MeOH and HCl (4 M in dioxane, 2 mL). The resulting mixture was heated at reflux for 1 h. The mixture was allowed to cool down to room temperature and was basified to pH=8 with saturated Na₂CO₃. The resulting mixture was extracted with EA (3×100 mL). The combined organic layers were washed with brine (2×100 mL), dried over anhydrous Na₂SO₄. After filtration, the filtrate was concentrated under reduced pressure. The residue was purified by silica gel column chromatography (eluting with 10:1 DCM/MeOH) to afford 3-(4-bromophenyl)-1,3-dimethyl-pyrrolidine (800 mg, 3.15 mmol, 70% yield) as a yellow oil. LCMS (ES, m/z): 254, 256 [M+H]⁺, Rt 0.509 min.

### Step 3. N-[4-(1,3-dimethylpyrrolidin-3-yl)phenyl]-1,1-diphenyl-methanimine

To a stirred mixture of 3-(4-bromophenyl)-1,3-dimethyl-pyrrolidine (670 mg, 2.64 mmol, 1 eq) and diphenylmethanimine (573.29 mg, 3.16 mmol, 530.83 μL, 1.2 eq) in dioxane (10 mL) were added Brettphos Pd-G3 (238.96 mg, 263.61 μmol, 0.1 eq), Brettphos (282.99 mg, 527.22 μmol, 0.2 eq) and cesium carbonate (1.72 g, 5.27 mmol, 2 eq) at room temperature. The resulting mixture was stirred for 2 h at 100° C. under the nitrogen atmosphere. The reaction was monitored by LCMS. The mixture was allowed to cool down to room temperature. The solids were filtered out and washed with EA (3×10 mL). The filtrate was concentrated under reduced pressure. The residue was purified by silica gel column chromatography (eluting with 1:1 EA/PE) to afford N-[4-(1,3-dimethylpyrrolidin-3-yl)phenyl]-1,1-diphenyl-methanimine (700 mg, 1.98 mmol, 75% yield) as a light-yellow oil. LCMS (ES, m/z): 355 [M+H]⁺, Rt 0.657 min.

### Step 4. N-[4-[(3S)-1,3-dimethylpyrrolidin-3-yl]phenyl]-1,1-diphenyl-methanimine and N-[4-[(3R)-1,3-dimethylpyrrolidin-3-yl]phenyl]-1,1-diphenyl-methanimine

The mixture of N-[4-(1,3-dimethylpyrrolidin-3-yl)phenyl]-1,1-diphenyl-methanimine (700 mg, 1.97 mmol, 1 eq) was separated by Prep-CHIRAL-HPLC with the following conditions (Column: CHIRALPAK IH-3, 3.0×50 mm, 3 μm; Mobile Phase B: IPA (0.1% DEA); Flow rate: 2 mL/min; Gradient: isocratic 10% B; Wave Length: 220 nm) to afford N-[4-[(3S)-1,3-dimethylpyrrolidin-3-yl]phenyl]-1,1-diphenyl-methanimine (250 mg, 0.71 mmol, 36% yield) as a yellow oil and N-[4-[(3R)-1,3-dimethylpyrrolidin-3-yl]phenyl]-1,1-diphenyl-methanimine (250 mg, 0.71 mmol, 36% yield) as a yellow oil. LCMS (ES, m/z): 355 [M+H]⁺, Rt 0.657 min.

The absolute stereochemistry was arbitrarily assigned following the chiral HPLC separation.

### Step 5. Intermediates 95 and 96: (S)-4-(1,3-dimethylpyrrolidin-3-yl)aniline and (R)-4-(1,3-dimethylpyrrolidin-3-yl)aniline

Imine hydrolysis for each individual enantiomer obtained in step 4 above was carried out according to the general procedure below:

A solution of each individual separated enantiomer (50 mg, 141.05 μmol, 1 eq.) in THF (3 mL), AcOH (1 mL) and water (0.5 mL) was stirred for 1 h at 100° C. The resulting mixture was concentrated under reduced pressure to afford either (S)-4-(1,3-dimethylpyrrolidin-3-yl)aniline or (R)-4-(1,3-dimethylpyrrolidin-3-yl)aniline (35 mg, crude) as a yellow oils. The crude products were used in the next step directly without further purification.

LCMS (ES, m/z): 191 [M+H]⁺, Rt 0.266 min.

### Intermediate 97 and Intermediate 98. tert-butyl (R)-2-(4-aminophenyl)pyrrolidine-1-carboxylate and tert-butyl (S)-2-(4-aminophenyl)pyrrolidine-1-carboxylate

### Step 1. tert-butyl 2-(4-aminophenyl)pyrrolidine-1-carboxylate

To a solution of 4-bromoaniline (1.71 g, 9.94 mmol, 1 eq.) in THF (70 mL) was added s-BuLi (1.3 M in hexane, 9.94 mL, 1.3 eq.) dropwise at −30° C. under nitrogen atmosphere. The mixture was stirred for 5 min at −30° C. To above solution was added ZnCl₂ (1 M, 5.96 mL, 0.6 eq.) dropwise at −30° C. The mixture was stirred for additional 0.5 h at −30° C. and allowed to warm to room temperature and stirred for more 0.5 h. To the above solution was added tert-butyl pyrrolidine-1-carboxylate (1.19 g, 6.96 mmol, 0.7 eq.), Pd(OAc)₂ (109 mg, 497.03 umol, 0.05 eq.) and tBu₃PHBF₄ (109 mg, 621.28 umol, 0.0625 eq.). The mixture was stirred for additional 16 h at room temperature. The reaction was monitored by LCMS. Then 35% NH₄OH (100 ml) was added and the resulting solution was stirred at room temperature for 1 h. The solids were removed by filtration through Celite and washed with EA (100 ml). The filtrate was washed with HCl (100 ml, 1M) and saturated brine (200 ml), dried over Na₂SO₄ and concentrated under reduced pressure. The residue was purified by silica gel column chromatography (eluting with 1:2 EA/PE) to afford tert-butyl 2-(4-aminophenyl)pyrrolidine-1-carboxylate (1.0 g, 3.61 mmol, 38% yield) as yellow solid. LCMS (ES, m/z): 263[M+H]⁺, Rt 0.542 min.

### Step 2. Intermediates 97 and 98: tert-butyl (R)-2-(4-aminophenyl)pyrrolidine-1-carboxylate and tert-butyl (S)-2-(4-aminophenyl)pyrrolidine-1-carboxylate

The racemic product tert-butyl 2-(4-aminophenyl)pyrrolidine-1-carboxylate (1.0 g, 3.61 mmol, 1 eq.) was separated by the following conditions: (Column: OptiChiral-C9-5, 3×25 cm, 5 μm; Mobile Phase A: CO₂, Mobile Phase B: MeOH (0.1% 2M NH₃-MEOH); Flow rate: 100 mL/min; Gradient: isocratic 30% B; Column Temperature (° C.): 35; Back Pressure(bar): 100; Wave Length: 220 nm; RT1(min): 3.1; RT2(min): 4.7; Sample Solvent: ACN:MeOH=4:1) to afford tert-butyl (R)-2-(4-aminophenyl)pyrrolidine-1-carboxylate (0.44 g, 1.59 mmol, 32% yield) as a yellow solid and tert-butyl (S)-2-(4-aminophenyl)pyrrolidine-1-carboxylate (0.44 g, 1.59 mmol, 32% yield) as a yellow solid. LCMS (ES, m/z): 263 [M+H]⁺, Rt 0.542 min.

The absolute stereochemistry was arbitrarily assigned following the chiral HPLC separation.

### Intermediate 99 and Intermediate 100. tert-butyl (R)-2-(5-aminopyridin-2-yl)pyrrolidine-1-carboxylate and tert-butyl (S)-2-(5-aminopyridin-2-yl)pyrrolidine-1-carboxylate

### Step 1. tert-butyl 2-(5-bromo-2-pyridyl)pyrrolidine-1-carboxylate

A mixture of 2,5-dibromopyridine (500 mg, 2.11 mmol, 1 eq.), 1-tert-butoxycarbonylpyrrolidine-2-carboxylic acid (681.47 mg, 3.17 mmol, 1.5 eq.), Ir[(dF(Me)(ppy)]₂(dtppy)PF₆ (21.44 mg, 21.11 μmol, 0.01 eq.), 5,5′-dimethyl-2′-bipyridine (58.25 mg, 316.60 μmol, 0.15 eq.), dibromonickel 1,2-dimethoxyethane (65.14 mg, 211.07 μmol, 0.1 eq.) and Cs₂CO₃ (1.38 g, 4.22 mmol, 2 eq.) in DMF (36 mL) was irradiated with blue light (450 nm) for 1 h at 50° C. under N₂ atmosphere. The reaction was monitored by LC-MS. The resulting mixture was diluted with EA (100 mL) and washed with brine (3×20 ml). The organic layer was dried over anhydrous Na₂SO₄. After filtration, the filtrate was concentrated under reduced pressure. The residue was purified by silica gel column chromatography (eluting with 1:4 EA/PE) to afford tert-butyl 2-(5-bromo-2-pyridyl)pyrrolidine-1-carboxylate (150 mg, 458.41 μmol, 22% yield) as a white solid. LCMS (ES, m/z): 327, 329 [M+H]⁺, Rt 0.781 min.

¹H NMR (300 MHz, Chloroform-d) δ 8.59 (d, J=2.4 Hz, 1H), 7.77-7.73 (m, 1H), 7.09 (t, J=7.6 Hz, 1H), 4.93-4.81 (m, 1H), 3.65-3.55 (m, 2H), 2.4-2.29 (m, 1H), 1.92-1.86 (m, 3H), 1.24 (d, J=8.4 Hz, 9H).

### Step 2. tert-butyl 2-[5-(benzhydrylideneamino)-2-pyridyl]pyrrolidine-1-carboxylate

A mixture of tert-butyl 2-(5-bromo-2-pyridyl)pyrrolidine-1-carboxylate (600 mg, 1.83 mmol, 1 eq.), diphenylmethanimine (664.63 mg, 3.67 mmol, 615.40 μL, 2 eq.), BrettPhos (196.93 mg, 366.73 μmol, 0.2 eq.), BrettPhos Pd G3 (166.50 mg, 183.37 μmol, 0.1 eq.) and Cs₂CO₃ (1.19 g, 3.67 mmol, 2 eq.) in dioxane (10 mL) was stirred for 2 h at 100° C. under N₂ atmosphere. The reaction was monitored by LC-MS. The mixture was allowed to cool down to room temperature. The solids were filtered and washed with EA (2×10 mL). The filtrate was concentrated under reduced pressure. The residue was purified by silica gel column chromatography (eluting with 1:2 EA/PE) to afford tert-butyl 2-[5-(benzhydrylideneamino)-2-pyridyl]pyrrolidine-1-carboxylate (600 mg, 1.40 mmol, 77% yield) as a white solid. LCMS (ES, m/z): 428 [M+H]⁺, Rt 0.721 min.

### Step 3. tert-butyl 2-(5-amino-2-pyridyl)pyrrolidine-1-carboxylate

A mixture of tert-butyl 2-[5-(benzhydrylideneamino)-2-pyridyl]pyrrolidine-1-carboxylate (600 mg, 1.40 mmol, 1 eq.), palladium on carbon (120 mg, 10 wt %) and ammonium formate (442.46 mg, 7.02 mmol, 5 eq.) in mixed solvents of MeOH (5 mL) and THF (5 mL) was stirred at 60° C. under nitrogen atmosphere for 4 h. The reaction was monitored by LC-MS. The mixture was allowed to cool down to room temperature. The solids were filtered and washed with EA (2×10 mL). The filtrate was concentrated under reduced pressure. The residue was purified by silica gel column chromatography (eluting with 1:1 EA/PE) to afford tert-butyl 2-(5-amino-2-pyridyl)pyrrolidine-1-carboxylate (300 mg, 1.14 mmol, 81% yield) as a yellow solid. LCMS (ES, m/z): 264 [M+H]⁺, Rt 0.523 min.

¹H NMR (300 MHz, Chloroform-d) δ 8.04-8.00 (m, 1H), 7.02-6.91 (m, 2H), 4.91-4.78 (m, 1H), 3.63-3.55 (m, 2H), 2.34-2.22 (m, 2H), 1.89-1.83 (m, 2H), 1.24 (d, J=8.4 Hz, 9H).

### Step 4. tert-butyl (R)-2-(5-aminopyridin-2-yl)pyrrolidine-1-carboxylate and

### tert-butyl (S)-2-(5-aminopyridin-2-yl)pyrrolidine-1-carboxylate (Intermediate 99 and Intermediate 100)

The racemic product tert-butyl 2-(5-aminopyridin-2-yl)pyrrolidine-1-carboxylate (600 mg, 2.28 mmol, 1 eq.) was separated by Prep-CHIRAL-HPLC with the following conditions (Column: CHIRALPAK IA-3, 4.6*50 mm, 3 μm; Mobile Phase A: MtBE (0.5% IPAmine):IPA=70:30; Flow rate: 1 mL/min; Gradient: 0% B to 0% B; Injection Volume: 5 mL) to afford tert-butyl (R)-2-(5-aminopyridin-2-yl)pyrrolidine-1-carboxylate (250 mg, 0.95 mmol, 42% yield) as a yellow solid and tert-butyl (S)-2-(5-aminopyridin-2-yl)pyrrolidine-1-carboxylate (240 mg, 0.91 mmol, 40% yield) as a yellow solid. LCMS (ES, m/z): 264 [M+H]⁺, Rt 0.523 min.

The absolute stereochemistry was arbitrarily assigned following the chiral HPLC separation.

### Intermediate 101 and Intermediate 102. tert-butyl (3R)-3-(4-amino-2-methyl-phenyl)morpholine-4-carboxylate and tert-butyl (3S)-3-(4-amino-2-methyl-phenyl)morpholine-4-carboxylate

### Step 1. 2-(trimethylsilylmethoxy)ethyl 4-methylbenzenesulfonate

A mixture of trimethylsilylmethyl trifluoromethanesulfonate (5 g, 21.16 mmol, 1 eq.) in ethylene glycol (16.65 g, 268.26 mmol, 15 mL, 13.5 eq.) was stirred at room temperature for 16 h. The reaction was monitored by LC-MS. Then ice/water (50 mL) was added. The mixture was extracted with EA (3×50 mL). The organic layers were combined, dried over sodium sulfate, filtered and concentrated. The residue was dissolved in DCM (20 mL). Then N,N-dimethylpyridin-4-amine (231.53 mg, 1.90 mmol, 0.1 eq), N,N-diethylethanamine (2.11 g, 20.85 mmol, 2.91 mL, 1 eq.) and 4-methylbenzenesulfonyl chloride (3.61 g, 18.95 mmol, 0.9 eq.) were added in portions. The mixture was stirred at room temperature for 16 h. The reaction was monitored by LC-MS. Then ice/water (50 mL) was added. The mixture was extracted with DCM (3×50 mL). The organic layers were combined, dried over Na₂SO₄, filtered and concentrated. The residue was purified by silica gel column chromatography (eluting with 10:1 PE/EA) to afford 2-(trimethylsilylmethoxy)ethyl 4-methylbenzenesulfonate (4.1 g, 13.56 mmol, 64% yield) as a colorless oil. LCMS (ES, m/z): 303 [M+H]⁺, Rt 0.913 min.

### Step 2. 2-[2-(Trimethylsilylmethoxy)ethyl]isoindoline-1,3-dione

A mixture of (1,3-dioxoisoindolin-2-yl)potassium (3.03 g, 16.35 mmol, 1.15 eq.) and 2-(trimethylsilylmethoxy)ethyl 4-methylbenzenesulfonate (4.3 g, 14.22 mmol, 1 eq.) in N,N-dimethylformamide (80 mL) was stirred for 2 h at 100° C. The reaction was monitored by TLC. The mixture was cooled to room temperature. Then water (100 mL) was added. The mixture was extracted with DCM (80 mL×3). The organic layers were combined, washed with brine (50 mL×3), dried over Na₂SO₄, filtered and concentrated. The residue was purified by silica gel column chromatography (eluting with 5:1 PE/EA) to afford 2-[2-(trimethylsilylmethoxy)ethyl]isoindoline-1,3-dione (3.73 g, 13.45 mmol, 95% yield) as a colorless oil. LCMS (ES, m/z): 278 [M+H]⁺, Rt 0.900 min.

### Step 3. 2-(trimethylsilylmethoxy)ethanamine

A solution of 2-[2-(trimethylsilylmethoxy)ethyl]isoindoline-1,3-dione (2.7 g, 9.73 mmol, 1 eq.) in methanol (60 mL) was added hydrazine hydrate solution (35%) (4.73 mL, 97.34 mmol, 4.73 mL, 10 eq.). The mixture was stirred at 70° C. for 1 h. The reaction was monitored by TLC. After cooling to room temperature, water (100 mL) was added. The mixture was stirred at room temperature till the white solid was dissolved. The mixture was extracted with DCM (100 mL×3). The organic layers were combined, washed with water (100 mL) and brine (100 mL), dried over Na₂SO₄, filtered and concentrated to afford 2-(trimethylsilylmethoxy)ethanamine (1.35 g, 9.17 mmol, 94% yield) as a light-yellow oil. LCMS (ES, m/z): 148 [M+H]⁺, Rt 0.515 min.

### Step 4. 3-(2-methyl-4-nitro-phenyl)morpholine

A mixture of 2-(trimethylsilylmethoxy)ethanamine (1.45 g, 9.84 mmol, 1 eq.) and 2-methyl-4-nitro-benzaldehyde (1.63 g, 9.84 mmol, 1 eq.) in acetonitrile (40 mL) was added 4A molecular sieves (900 mg). The mixture was stirred at room temperature for 5 h. The solids were filtered out. The filtrate was concentrated to afford 1-(2-methyl-4-nitro-phenyl)-N-[2-(trimethylsilylmethoxy)ethyl]methanimine (2.7 g, crude) as a light-yellow oil.

A mixture of 1-(2-methyl-4-nitro-phenyl)-N-[2-(trimethylsilylmethoxy)ethyl]methanimine (1 g, 3.40 mmol, 1 eq.) in toluene (9.0 mL) and 1,1,1,3,3,3-Hexafluoro-2-propanol (1.0 mL) were added 2,4,6-triphenylpyrylium tetrafluoroborate (134.56 mg, 339.65 umol, 0.1 eq.) and trimethylsilyl trifluoromethanesulfonate (1.51 g, 6.79 mmol, 2 eq.). The mixture was stirred at room temperature for 16 h under blue light. The reaction was monitored by LCMS. Then DCM (100 mL) and NH₄OH solution (10%, 25 mL) was added. The phases were separated and the aqueous phase was extracted with DCM (3×100 mL). The organic phases were combined, washed with brine, dried over MgSO₄, filtered and concentrated under reduced pressure. The residue was purified by silica gel column chromatography (eluting with 10:1 DCM:MeOH) to afford 3-(2-methyl-4-nitro-phenyl)morpholine (340 mg, 1.53 mmol, 45% yield) as a brown oil. LCMS (ES, m/z): 223 [M+H]⁺, Rt 0.301 min.

### Step 5. tert-butyl 3-(2-methyl-4-nitro-phenyl)morpholine-4-carboxylate

To a stirred solution of 3-(2-methyl-4-nitro-phenyl)morpholine (300 mg, 1.35 mmol, 1 eq.), N,N-dimethylpyridin-4-amine (16.49 mg, 134.99 umol, 0.1 eq.) and N,N-diethylethanamine (188.15 uL, 1.35 mmol, 1 eq.) in DCM (20 mL) was added Boc₂O (441.92 mg, 2.02 mmol, 1.5 eq.) portionwise. The resulting mixture was stirred for 4 h at room temperature. The reaction was monitored by TLC. The resulting mixture was diluted with water (20 mL) and was extracted with DCM (2×30 ml). The combined organic layers were washed with brine (2×30 ml), dried over anhydrous Na₂SO₄. After filtration, the filtrate was concentrated under reduced pressure. The residue was purified by silica gel column chromatography (eluting with 2:1 PE:EA) to afford tert-butyl 3-(2-methyl-4-nitro-phenyl)morpholine-4-carboxylate (300 mg, 837.58 umol, 62% yield) as a yellow solid. LCMS (ES, m/z): 323 [M+H]⁺. Rt 1.277 min.

### Step 6. tert-butyl (3R)-3-(2-methyl-4-nitro-phenyl)morpholine-4-carboxylate and tert-butyl (3S)-3-(2-methyl-4-nitro-phenyl)morpholine-4-carboxylate

Racemic tert-butyl 3-(2-methyl-4-nitro-phenyl)morpholine-4-carboxylate (800 mg, 2.48 mmol, 1 eq.) was separated by prep-HPLC with the following conditions: (Column: CHIRAL ART Cellulose-SC, 2*25 cm, 5 μm; Mobile Phase A: Hex (0.5% 2M NH₃-MeOH)-HPLC, Mobile Phase B: EtOH-HPLC; Flow rate: 20 mL/min; Gradient: 15% B to 15% B in 20 min; Wave Length: 220/254 nm; RT1(min): 11.323; RT2(min): 16.352; Sample Solvent: EtOH-HPLC; Injection Volume: 1.2 mL; Number Of Runs: 5 to afford tert-butyl (3R)-3-(2-methyl-4-nitro-phenyl)morpholine-4-carboxylate (250 mg, 31%) as a colorless oil and tert-butyl (3S)-3-(2-methyl-4-nitro-phenyl)morpholine-4-carboxylate (280 mg, 35%) as a colorless oil. LCMS (ES, m/z): 323 [M+H]⁺. Rt 1.277 min.

The absolute stereochemistry was arbitrarily assigned following the chiral HPLC separation.

### Step 7. tert-butyl (3R)-3-(4-amino-2-methyl-phenyl)morpholine-4-carboxylate (Intermediate 101)

To a solution of tert-butyl (3R)-3-(2-methyl-4-nitro-phenyl)morpholine-4-carboxylate (250 mg, 775.54 umol, 1 eq.) in EtOH (5 mL) was added Pd/C (50 mg, 10 wt %) in portions at room temperature under N₂ atmosphere. The resulting mixture was stirred for 4 h at room temperature under the H₂ atmosphere. Desired product could be detected by LCMS. The solids were filtered out and washed with EtOH (3×5 mL). The filtrate was concentrated under reduced pressure. The residue was purified by reverse flash chromatography with the following conditions: (column, C18 silica gel; mobile phase: A, water (containing 0.1M NH₄HCO₃) and B, ACN (5% to 60%) over 60 min; detector, UV 254 nm) to afford tert-butyl (3R)-3-(4-amino-2-methyl-phenyl)morpholine-4-carboxylate (62 mg, 212.06 umol, 27% yield) as a yellow oil. LCMS (ES, m/z): 293 [M+H]⁺. Rt 0.633 min.

### Step 8. tert-butyl (3S)-3-(4-amino-2-methyl-phenyl)morpholine-4-carboxylate (Intermediate 102)

To a stirred solution of tert-butyl (3S)-3-(2-methyl-4-nitro-phenyl)morpholine-4-carboxylate (280 mg, 868.61 umol, 1 eq.) in EtOH (10 mL) was added Pd/C (60 mg, 10 wt %) in portions at room temperature under N₂ atmosphere. The resulting mixture was stirred for 4 h at 25° C. under the H₂ atmosphere. The reaction was monitored by LC-MS. The solids were filtered out and washed with EtOH (3×5 mL). The filtrate was concentrated under reduced pressure to afford tert-butyl (3S)-3-(4-amino-2-methyl-phenyl)morpholine-4-carboxylate (220 mg, crude) as a yellow oil. LCMS (ES, m/z): 293 [M+H]⁺. Rt 0.811 min.

### Intermediate 103. tert-Butyl 3-(4-aminophenyl)azetidine-1-carboxylate

To a stirred mixture of tert-butyl 3-iodoazetidine-1-carboxylate (500 mg, 1.77 mmol, 1 eq.) in isopropyl alcohol (10 mL) was added (4-aminophenyl)boronic acid (314.42 mg, 2.30 mmol, 1.3 eq.), (1R,2S)-2-aminocyclohexanol hydrochloride (16.07 mg, 105.97 μmol, 0.06 eq.) and diiodonickel (33.11 mg, 105.97 μmol, 0.06 eq.) in portions. The resulting mixture was stirred for 1 min at 0° C. under N₂ atmosphere. To the above mixture was added sodium bis(trimethylsilyl)azanide (1.62 g, 8.83 mmol, 5 eq.) at 0° C. under N₂ atmosphere. The resulting mixture was stirred for 2 h at 150° C. under N₂ atmosphere under microwave irradiation. The reaction was monitored by LC-MS. The resulting mixture was concentrated under reduced pressure. The resulting mixture was washed with brine (40 mL) and was extracted with EA (3×40 mL). The combined organic layers were dried over anhydrous Na₂SO₄. The resulting mixture was concentrated under reduced pressure. The residue was purified by silica gel column chromatography (eluting with 3:1 EA/PE) to afford tert-butyl 3-(4-aminophenyl)azetidine-1-carboxylate (88 mg, 354.38 μmol, 20% yield) as a brown solid.

¹H NMR (300 MHz, DMSO-d6) δ 6.98 (d, J=8.4 Hz, 2H), 6.54 (d, J=8.1 Hz, 2H), 4.99 (br, 2H), 4.21-4.12 (m, 2H), 3.81-3.54 (m, 3H), 1.40 (s, 9H). LCMS (ES, m/z): 193 [M+H-56]⁺, Rt 0.577 min.

### Intermediate 104. tert-Butyl 3-(4-amino-2-fluorophenyl)azetidine-1-carboxylate

Step 1. (1-(tert-butoxycarbonyl)azetidin-3-yl)zinc(II) iodide To a solution of tert-butyl 3-iodoazetidine-1-carboxylate (1.26 g, 4.45 mmol, 1 eq.) in DMAc (15 mL) were added Zn (436 mg, 6.68 mmol, 1.5 eq.), TMSCl (726 mg, 6.68 mmol, 1.5 eq.) and 1,2-dibromoethane (860 mg, 4.45 mmol, 1 eq.). The mixture was stirred for 0.5 h at 65° C. under nitrogen atmosphere. Then the reaction was allowed to cool down to room temperature. The mixture solution was used in the next step directly without further purification.

### Step 2. tert-Butyl 3-(2-fluoro-4-nitrophenyl)azetidine-1-carboxylate

To a solution of 1-bromo-2-fluoro-4-nitrobenzene (900 mg, 4.11 mmol, 1 eq.) in DMAc (10 mL) were added Pd(dppf)Cl₂ (298 mg, 0.411 mmol, 0.1 eq.), CuI (783 mg, 4.11 mmol, 1 eq.) and above solution of (1-(tert-butoxycarbonyl)azetidin-3-yl)zinc(II) iodide in DMAc (15 mL) dropwise. The mixture was stirred for 2 h at 85° C. under the N₂ atmosphere. The reaction was monitored by LC-MS. The mixture was allowed to cool down to room temperature and quenched by the addition of water (100 mL). The resulting mixture was extracted with EA (3×80 mL). The combined organic layers were washed with brine (100 mL), dried over anhydrous Na₂SO₄. After filtration, the filtrate was concentrated under reduced pressure. The residue was purified by silica gel column chromatography (eluting with 1:1 EA/PE) to afford tert-butyl 3-(2-fluoro-4-nitrophenyl)azetidine-1-carboxylate (750 mg, 2.53 mmol, 61% yield) as yellow solid. LCMS (ES, m/z): 297 [M+H]⁺, Rt 0.689 min.

¹H NMR (300 MHz, DMSO-d6) δ 8.10-8.06 (m, 1H), 7.95-7.91 (m, 1H), 7.61-7.56 (m, 1H), 4.44-4.35 (m, 2H), 4.13-4.01 (m, 3H), 1.47 (s, 9H).

### Step 3. tert-butyl 3-(4-amino-2-fluorophenyl)azetidine-1-carboxylate (Intermediate 104)

To a solution of tert-butyl 3-(2-fluoro-4-nitrophenyl)azetidine-1-carboxylate (620 mg, 2.09 mmol, 1 eq.) in HOAc (10 mL) were added Zn (548 mg, 8.36 mmol, 4 eq.) and NH₄Cl (1.12 g, 20.9 mmol, 10 eq.). The mixture was stirred for 3 h at 80° C. The reaction was monitored by LC-MS. The reaction was allowed to cool down to room temperature. The resulting mixture was filtered. The filter cake was washed with AcOH (2 mL). The filtrate was concentrated under reduced pressure. The residue was purified by silica gel column chromatography (eluting with 10:1 DCM/MeOH) to afford tert-butyl 3-(4-amino-2-fluorophenyl)azetidine-1-carboxylate (500 mg, 1.88 mmol, 89% yield) as a yellow solid. LCMS (ES, m/z): 211 [M+H-56]⁺. Rt 0.200 min.

### Intermediate 105. tert-Butyl 3-(5-amino-2-pyridyl)azetidine-1-carboxylate

### Step 1. tert-butyl 3-(5-nitro-2-pyridyl)azetidine-1-carboxylate

To a stirred mixture of 2-bromo-5-nitropyridine (0.9 g, 4.47 mmol, 1 eq.) and (1-(tert-butoxycarbonyl)azetidin-3-yl)zinc(II) iodide (Intermediate 104, step 1, 1.86 g, 5.37 mmol, 1.2 eq.) in DMAc (15 mL) were added Pd(dppf)C12 (327.76 mg, 0.45 mmol, 0.1 eq.) and CuI (170 mg, 0.89 mmol, 0.2 eq). The resulting mixture was stirred for 2 h at 80° C. under the N₂ atmosphere. The reaction was monitored by LC-MS. The mixture was cooled down to room temperature. Then water (50 mL) was added. The resulting mixture was extracted with EA (3×50 mL). The combined organic layers were washed with brine (2×50 mL), dried over anhydrous Na₂SO₄. After filtration, the filtrate was concentrated under reduced pressure. The residue was purified by silica gel column chromatography (eluting with 3:1 PE/EA) to afford tert-butyl 3-(5-nitropyridin-2-yl)azetidine-1-carboxylate (750 mg, 2.21 mmol, 55% yield) as a yellow solid. LCMS (ES, m/z): 224 [M+H-56]⁺, Rt 0.889 min.

¹H NMR (300 MHz, Chloroform-d) δ 9.45 (d, J=2.7 Hz, 1H), 8.47-8.43 (m, 1H), 7.44 (d, J=8.4 Hz, 1H), 4.38-4.32 (m, 2H), 4.21-4.11 (m, 2H), 4.05-3.95 (m, 1H), 1.47 (s, 9H).

### Step 2. tert-butyl 3-(5-amino-2-pyridyl)azetidine-1-carboxylate (Intermediate 105)

To a solution of tert-butyl 3-(5-nitro-2-pyridyl)azetidine-1-carboxylate (600 mg, 2.15 mmol, 1 eq.) in EtOH (20 mL) was added palladium on carbon (120 mg, 10 wt %). The mixture was stirred at room temperature for 2 h under the hydrogen atmosphere using a hydrogen balloon. The reaction was monitored by LC-MS. The solids were filtered out and washed with EtOH (3×5 mL). The resulting mixture was concentrated under reduced pressure to afford tert-butyl 3-(5-amino-2-pyridyl)azetidine-1-carboxylate (550 mg, crude) as a yellow oil. LCMS (ES, m/z): 250 [M+H]⁺, Rt 0.943 min.

### Intermediate 106. tert-Butyl 3-(4-amino-2-chlorophenyl)azetidine-1-carboxylate

Prepared in an analogous manner as intermediate 105. LCMS (ES, m/z): 268, 270 [M+H-56+41]+, Rt 0.787 min. ¹H NMR (300 MHz, DMSO-d6) δ 7.13 (d, J=8.4 Hz, 1H), 6.61 (s, 1H), 6.54 (d, J=8.4 Hz, 1H), 4.22-4.09 (m, 2H), 3.93-3.79 (m, 3H), 1.38 (s, 9H).

### Intermediate 109. 2-Methylspiro[1,3-dihydroisoquinoline-4,1′-cyclopropane]-7-amine

### Step 1. Methyl 1-(2-cyanophenyl)cyclopropanecarboxylate

To a stirred solution of methyl 2-(2-cyanophenyl)acetate (26.8 g, 152.98 mmol, 1 eq.) and tetrabutylammonium bromide (54.25 g, 168.28 mmol, 1.1 eq.) in toluene (216 mL) was added sodium hydroxide solution (50%, 107 mL, 9 eq.) dropwise at room temperature. The resulting mixture was stirred for 1 h at room temperature. The reaction was monitored by LC-MS. The reaction mixture was diluted with water, and extracted with ethyl acetate. The organic phases were combined and washed with brine. The organic phase was dried over anhydrous Na₂SO₄. After filtration, the filtrate was concentrated under reduced pressure. The residue was purified by silica gel column chromatography (eluting with 5:1 PE/EA) to afford methyl 1-(2-cyanophenyl)cyclopropanecarboxylate (22 g, 98.40 mmol, 64% yield) as a white solid. LCMS (ES, m/z): 202 [M+H]⁺, Rt 1.308 min.

### Step 2. Methyl 1-[2-(aminomethyl)phenyl]cyclopropanecarboxylate

To a stirred solution of methyl 1-(2-cyanophenyl)cyclopropanecarboxylate (11 g, 54.67 mmol, 1 eq.) in EtOH (85 mL) was added Pd/C (6.64 g, 5.47 mmol, 10 wt %) in portions at room temperature under N₂ atmosphere. The resulting mixture was stirred for 3 h at room temperature under the H₂ atmosphere. The reaction was monitored by TLC. After filtration, the filtrate was concentrated under reduced pressure. The crude product was washed with diethyl ether to obtain methyl 1-[2-(aminomethyl)phenyl]cyclopropanecarboxylate;hydrochloride (12 g, crude) as a white solid. LCMS (ES, m/z): 206 [M+H]⁺, Rt 0.373 min.

### Step 3. Spiro[1,2-dihydroisoquinoline-4,1′-cyclopropane]-3-one

To a stirred solution of methyl 1-[2-(aminomethyl)phenyl]cyclopropanecarboxylate (12 g, 49.65 mmol, 1 eq.) in MeOH (180 mL) was added NaOH (5 M, 12.97 mL, 1.31 eq) dropwise at room temperature under N₂ atmosphere. The resulting mixture was stirred for 0.5 h at room temperature. The reaction was monitored by LC-MS. The mixture was neutralized with aqueous 1 M hydrochloric acid. Then methanol was removed under reduced pressure. The residue was diluted with water, and extracted three times with EA. The organic layer was washed with brine, dried with anhydrous Na₂SO₄. After filtration, the filtrate was concentrated under reduced pressure to obtain spiro[1,2-dihydroisoquinoline-4,1′-cyclopropane]-3-one (8.5 g, crude) as a white solid. LCMS (ES, m/z): 174 [M+H]⁺, Rt 0.565 min.

### Step 4. 7-Nitrospiro[1,2-dihydroisoquinoline-4,1′-cyclopropane]-3-one

Potassium nitrate (5.26 g, 52.02 mmol, 1.06 eq.) was added over 5 mins to a sulfuric acid solution (80 mL) of spiro[1,2-dihydroisoquinoline-4,1′-cyclopropane]-3-one (8.5 g, 49.07 mmol, 1 eq.). The reaction was stirred for 10 mins at room temperature. The reaction was monitored by LC-MS. Then the mixture was poured into cold water. The precipitate was collected via filtration and washed with water to obtain 7-nitrospiro[1,2-dihydroisoquinoline-4,1′-cyclopropane]-3-one (9 g, crude). LCMS (ES, m/z): 219 [M+H]⁺, Rt 0.567 min.

### Step 5. 7′-Nitro-2′,3′-dihydro-1′H-spiro[cyclopropane-1,4′-isoquinoline]

Into a solution of NaBH₄ (4.68 g, 123.73 mmol, 3 eq.) in THF (42 mL) was added BF₃-Et₂O (20.09 mL, 82.49 mmol, 47% purity, 2 eq.) dropwise. The mixture was stirred for 1 h at room temperature. To the above, 7-nitrospiro[1,2-dihydroisoquinoline-4,1′-cyclopropane]-3-one (9 g, 41.24 mmol, 1 eq.) in THF (35 mL) was added, and heated to 70° C. for 2 h. The reaction was monitored by LC-MS. The reaction mixture was cooled to room temperature and then neutralized with saturated Na₂CO₃. The solvent was removed. The residue was dissolved in ethanol and HCl (5 M). The mixture was heated under reflux for 1 h. The reaction was cooled, then the solvent was evaporated under reduced pressure. The residue was neutralized with saturated aqueous K₂CO₃. The aqueous layer was extracted with DCM (3×100 mL), dried over anhydrous Na₂SO₄, filtered and concentrated under reduced pressure to yield 7-nitrospiro[2,3-dihydro-1H-isoquinoline-4,1′-cyclopropane](8 g, crude) as a brown solid. LCMS (ES, m/z): 205 [M+H]⁺, Rt 0.478 min.

Step 6. 2-Methyl-7-nitro-spiro[1,3-dihydroisoquinoline-4,1′-cyclopropane] Sodium cyanoborohydride (4.92 g, 78.35 mmol, 2 eq.) was added to a solution of 7-nitrospiro[2,3-dihydro-1H-isoquinoline-4,1′-cyclopropane](8 g, 39.17 mmol, 1 eq) in MeOH (190 mL). Then formaldehyde (10.69 g, 117.52 mmol, 33% purity, 3 eq.) and acetic acid (2.35 g, 39.17 mmol, 1 eq.) were added. The reaction was stirred at room temperature for 4 h. The reaction was monitored by LC-MS. The reaction was neutralized with sat. NaHCO₃. The solvent was evaporated under reduced pressure. The residue was diluted with water and extracted with MeOH:DCM (3×200 mL). The combined organic layers were concentrated under reduced pressure. The residue was purified by silica gel column chromatography (eluting with 10:1 DCM/MeOH) to afford 2-methyl-7-nitro-spiro[1,3-dihydroisoquinoline-4,1′-cyclopropane](5 g, 20.62 mmol, 53% yield) as a yellow solid. LCMS (ES, m/z): 219 [M+H]⁺, Rt 0.319 min.

### Step 7. 2-Methylspiro[1,3-dihydroisoquinoline-4,1′-cyclopropane]-7-amine (Intermediate 109)

To the mixture of 2-methyl-7-nitro-spiro[1,3-dihydroisoquinoline-4,1′-cyclopropane](1.8 g, 8.25 mmol, 1 eq.) and HCOONH₄ (5.20 g, 82.47 mmol, 10 eq.) in MeOH (80 mL) was added Pd/C (360.00 mg, 10 wt %). The reaction system was stirred at room temperature for 12 hours. The reaction was monitored by LC-MS. After filtration, the filtrate was diluted with saturated aqueous sodium carbonate solution. The solution was extracted with EA (3×100 mL). The organic layers were combined, washed with saturated brine and dried over anhydrous Na₂SO₄. After filtration, the filtrate was concentrated under reduced pressure. The crude product was purified by reverse flash chromatography with the following conditions: column, C18 silica gel; mobile phase: A, water and B, ACN (5% to 100%) over 30 min; detector, UV 254 nm to afford 2-methylspiro[1,3-dihydroisoquinoline-4,1′-cyclopropane]-7-amine (700 mg, 3.53 mmol, 43% yield) as a yellow solid. LCMS (ES, m/z): 189 [M+H]⁺, Rt 0.304 min.

### Intermediate 110: 2,4,4-Trimethyl-1,2,3,4-tetrahydroisoquinolin-7-amine

### Step 1. 2,4,4-Trimethyl-7-nitro-1,2,3,4-tetrahydroisoquinoline

To a stirred solution of 2,4,4-trimethyl-7-nitro-3,4-dihydroisoquinolin-1(2H)-one (Intermediate 48, step 3, 500 mg, 2.13 mmol, 1 eq.) in THF (10 mL) was added BH₃-THF (1 M in THF, 6.4 mL 6.4 mmol, 3 eq.) under N₂ atmosphere. The mixture was stirred at room temperature for 30 h. The reaction was monitored by LC-MS. The mixture was concentrated under reduced pressure. To the residue was added HCl (6 M, 8 mL) and stirred for 1 h at 100° C. The reaction was cooled to room temperature and concentrated under reduced pressure. The mixture was quenched with saturated NaHCO₃ (30 mL) and extracted with EA (3×30 mL). The combined organic layers were washed with brine (2×50 mL), dried over anhydrous Na₂SO₄. After filtration, the filtrate was concentrated under reduced pressure. The residue was purified by silica gel column chromatography (eluting with 1:1 EA/PE) to afford 2,4,4-trimethyl-7-nitro-1,2,3,4-tetrahydroisoquinoline (350 mg, 75% yield) as a yellow oil. ¹H NMR (300 MHz, DMSO-d6) δ 8.04-7.95 (m, 2H), 7.68-7.64 (m, 1H), 3.58 (s, 2H), 2.39 (s, 2H), 2.36 (s, 3H), 1.29 (s, 6H). LCMS (ESI+, m/z): 221 [M+H]⁺, Rt 0.575 min.

### Step 2. 2,4,4-trimethyl-1,2,3,4-tetrahydroisoquinolin-7-amine (Intermediate 110)

To a stirred solution of 2,4,4-trimethyl-7-nitro-1,3-dihydroisoquinoline (350 mg, 1.59 mmol) in ethanol (10 mL) was added Pd/C (70 mg, 10 wt %) under N₂ atmosphere. The resulting mixture was stirred for 16 h at room temperature under H₂ atmosphere. The reaction was monitored by LC-MS. The solids were filtered and washed with MeOH (2×15 mL). The filtrate was concentrated under reduced pressure. The crude product was purified by silica gel column chromatography (eluting with 10:1 DCM/MeOH) to afford 2,4,4-trimethyl-1,2,3,4-tetrahydroisoquinolin-7-amine (260 mg, 86% yield) as a yellow solid. LCMS (ESI+, m/z): 191 [M+H]⁺, Rt 0.278 min.

### Intermediate 111. tert-Butyl 3-amino-5,7-dihydropyrrolo[3,4-b]pyridine-6-carboxylate

### Step 1. tert-butyl 3-nitro-5,7-dihydropyrrolo[3,4-b]pyridine-6-carboxylate

A mixture of tert-butyl 3-oxopyrrolidine-1-carboxylate (1 g, 5.40 mmol, 1 eq.) and 1-methyl-3,5-dinitro-pyridin-2-one (1.29 g, 6.48 mmol, 1.2 eq.) in ammonia (7 M in MeOH, 10 mL) was stirred at 90° C. for 16 h. The reaction was monitored by LC-MS. The resulting mixture was concentrated under reduced pressure. The residue was purified by silica gel column chromatography (eluting with 1:1 EA/PE) to afford tert-butyl 3-nitro-5,7-dihydropyrrolo[3,4-b]pyridine-6-carboxylate (0.39 g, 1.47 mmol, 27% yield) as a yellow solid. LCMS (ES, m/z): 266 [M+H]⁺, Rt 0.830 min.

¹H NMR (300 MHz, Chloroform-d) δ 9.35 (s, 1H), 8.43-8.31 (m, 1H), 4.86-4.75 (m, 4H), 1.54 (s, 9H).

### Step 2. tert-butyl 3-amino-5,7-dihydropyrrolo[3,4-b]pyridine-6-carboxylate (Intermediate 111)

To a stirred mixture of tert-butyl 3-nitro-5,7-dihydropyrrolo[3,4-b]pyridine-6-carboxylate (150 mg, 565.47 μmol, 1 eq.) in MeOH (20 mL) were added Pd/C (30 mg, 10 wt %) under a nitrogen atmosphere. The resulting mixture was stirred for 1 h at room temperature under the nitrogen atmosphere. The reaction was monitored by LC-MS. The solids were filtered out and washed with MeOH (3×5 mL). The filtrate was concentrated under reduced pressure. The residue was purified by silica gel column chromatography (eluting with 2:1 EA/PE) to afford tert-butyl 3-amino-5,7-dihydropyrrolo[3,4-b]pyridine-6-carboxylate (100 mg, 425.02 μmol, 75% yield) as a yellow solid. LCMS (ES, m/z): 236 [M+H]⁺, Rt 0.507 min.

### Intermediate 113. tert-butyl (2S)-3-(4-aminophenyl)-2-[tert butoxycarbonyl(methyl)amino]propanoate

### Step 1. tert-butyl (2S)-2-(tert-butoxycarbonylamino)-3-(4-nitrophenyl)propanoate

To a stirred solution of (2S)-2-(tert-butoxycarbonylamino)-3-(4-nitrophenyl)propanoic acid (1 g, 3.23 mmol, 1 eq.) in tert-butanol (8 mL) were added Boc₂O (822.92 mg, 3.77 mmol, 1.2 eq.) and N,N-dimethylpyridin-4-amine (1.42 g, 11.60 mmol, 3.6 eq.) in portions. The resulting mixture was stirred for 2 h at room temperature. The reaction was monitored by LC-MS. Then water (50 mL) was added. The resulting mixture was extracted with EA (3×30 mL). The combined organic layers were washed with brine (2×50 mL), dried over anhydrous Na₂SO₄. After filtration, the filtrate was concentrated under reduced pressure. The residue was purified by silica gel column chromatography (eluting with 1:3 EA/PE) to afford tert-butyl (2S)-2-(tert-butoxycarbonylamino)-3-(4-nitrophenyl)propanoate (900 mg, 2.51 mmol, 76% yield) as a yellow oil. LCMS (ES, m/z): 252 [M+H-100-56+41]⁺, Rt 1.107 min.

### Step 2. tert-butyl (2S)-2-[tert-butoxycarbonyl(methyl)amino]-3-(4-nitrophenyl)propanoate

To a stirred solution of tert-butyl (2R)-2-(tert-butoxycarbonylamino)-3-(4-nitrophenyl)propanoate (880 mg, 2.40 mmol, 1 eq.) in DMF (10 mL) was added NaH (60% in oil, 144 mg, 3.60 mmol, 1.5 eq.) slowly at 0° C. over 10 minutes. Then iodomethane (409.07 mg, 2.88 mmol, 1.2 eq.) was added at 0° C. dropwise. The resulting mixture was stirred for 2 h at 0° C. A further batch of NaH (60% in oil, 96 mg, 2.40 mmol, 1 eq.) was added slowly. The resulting mixture was stirred for 10 minutes at 0° C. Iodomethane (340.89 mg, 1.87 mmol, 1 eq.) was added at 0° C. dropwise. The reaction mixture was stirred at 0° C. for another 1 h. The reaction was monitored by LC-MS. The reaction was quenched by saturated NH₄Cl (50 mL). The resulting mixture was extracted with EA (3×30 mL). The combined organic layers were washed with brine (2×50 mL), dried over anhydrous Na₂SO₄. After filtration, the filtrate was concentrated under reduced pressure. The crude was purified by silica gel column chromatography (eluting with 1:3 EA/PE) to afford tert-butyl (2S)-2-[tert-butoxycarbonyl(methyl)amino]-3-(4-nitrophenyl) propanoate (700 mg, 1.84 mmol, 75% yield) as a yellow oil. LCMS (ES, m/z): 381 [M+H]⁺, Rt 1.201 min.

### Step 3. tert-butyl (2S)-3-(4-aminophenyl)-2-[tert-butoxycarbonyl(methyl)amino]propanoate (Intermediate 113)

To a solution of tert-butyl (2R)-2-[tert-butoxycarbonyl(methyl)amino]-3-(4-nitrophenyl)propanoate (700 mg, 1.84 mmol, 1 eq.) in EtOH (10 mL) was added palladium on carbon (140 mg, 10 wt %). The mixture was hydrogenated at room temperature for 1 h using a hydrogen balloon. The reaction was monitored by LC-MS. The solids were filtered out and washed by EtOH (3×10 mL). The resulting mixture was concentrated under reduced pressure. The residue was purified by silica gel column chromatography (eluting with 1:1 EA/PE) to afford tert-butyl (2S)-3-(4-aminophenyl)-2-[tert-butoxycarbonyl(methyl)amino]propanoate (400 mg, 1.14 mmol, 62% yield) as a yellow oil. LCMS (ES, m/z): 351[M+H]⁺, Rt 1.377 min.

¹H NMR (300 MHz, DMSO-d6) δ 6.85 (d, J=7.5 Hz, 2H), 6.49 (d, J=7.8 Hz, 2H), 4.88 (br, 2H), 3.04-2.91 (m, 1H), 2.98-2.92 (m, 1H), 2.82-2.73 (m, 1H), 2.60 (s, 3H), 1.42-1.32 (m, 18H).

### Intermediate 114 tert-butyl (2R)-3-(4-aminophenyl)-2-[tert-butoxycarbonyl(methyl)amino]propanoate

Prepared according to the general procedure of Intermediate 113 using starting from (2R)-2-(tert-butoxycarbonylamino)-3-(4-nitrophenyl)propanoic acid. LCMS (ES, m/z): 351 [M+H]⁺, Rt 1.248 min.

### Intermediate 118. 6-tert-butylpyridin-3-amine

### Step 1. N-(6-tert-butyl-3-pyridyl)-1,1-diphenyl-methanimine

To a stirred solution of 5-bromo-2-tert-butyl-pyridine (600 mg, 2.80 mmol, 1 eq,) in dioxane (2 mL) were added diphenylmethanimine (609.46 mg, 3.36 mmol, 564.32 uL, 1.2 eq), Cs₂CO₃ (2.74 g, 8.41 mmol, 3 eq,), BrettPhos G3 (253.90 mg, 280.24 umol, 0.1 eq,) and BrettPhos (150.21 mg, 280.24 umol, 0.1 eq,). The resulting mixture was stirred for 1 h at 100° C. under the N₂ atmosphere. The reaction was monitored by LC-MS. The mixture was allowed to cool down to room temperature. The resulting mixture was filtered, the filter cake was washed with DCM (3×10 ml). The filtrate was concentrated under reduced pressure. The residue was purified by Prep-TLC (eluting with 1:1 EA/PE) to afford N-(6-tert-butyl-3-pyridyl)-1,1-diphenyl-methanimine (700 mg, 2.22 mmol, 79% yield) as a yellow oil. LCMS (ES, m/z): 315 [M+H]⁺, Rt 0.839 min.

### Step 2. 6-tert-butylpyridin-3-amine (Intermediate 118)

To a stirred solution of N-(6-tert-butyl-3-pyridyl)-1,1-diphenyl-methanimine (690 mg, 2.19 mmol, 1 eq) in tetrahydrofuran (9 mL) was added AcOH (3 mL) and H₂O (1.5 mL) dropwise. The resulting mixture was stirred for 1 h at 100° C. and monitored by LC-MS. The mixture was allowed to cool down to room temperature and was concentrated under vacuum. The crude product was purified by reverse phase chromatography (Column: C18; Mobile phase, A: water (containing 10 mmol/L NH₄HCO₃) and B: ACN (5% to 100% over 40 min); Detector, UV 254 nm) to afford 6-tert-butylpyridin-3-amine (220 mg, 1.45 mmol, 66% yield) as a yellow oil.

¹H NMR (300 MHz, DMSO-d6) δ 7.88 (d, J=5.4 Hz, 1H), 7.07-7.03 (m, 1H), 6.88-6.83 (m, 1H), 5.02 (br, 2H), 1.23 (s, 9H). LCMS (ES, m/z): 151 [M+H]⁺, Rt 0.506 min.

### Intermediate 124. 6-bromotriazolo[1,5-a]pyridine

### Step 1. N-[(E)-(5-bromo-2-pyridyl)methyleneamino]-4-methyl-benzenesulfonamide

The mixture of 5-bromopyridine-2-carbaldehyde (2 g, 10.75 mmol, 1 eq.) and 4-methylbenzenesulfonohydrazide (2.40 g, 12.90 mmol, 1.72 mL, 1.2 eq.) in MeOH (25 mL) was stirred at room temperature for 2 h. The reaction was monitored by LCMS. Then MeOH was removed. To the above was added water (50 mL). The mixture was extracted with EA (50 mL×3). The combined organic layer was dried with Na₂SO₄, filtered and concentrated to afford N-[(E)-(5-bromo-2-pyridyl)methyleneamino]-4-methyl-benzenesulfonamide (3 g, crude) as a yellow solid. LCMS (ES, m/z): 354, 356 [M+H]⁺. Rt 0.731 min.

### Step 2. 6-bromotriazolo[1,5-a]pyridine

A solution of N-[(E)-(5-bromo-2-pyridyl)methyleneamino]-4-methyl-benzenesulfonamide (3 g, 8.47 mmol) in NMM (20 mL) was stirred at 90° C. for 2 h. The reaction was monitored by LCMS. The reaction was cooled down to room temperature. The mixture was concentrated under reduced pressure. The residue was purified by silica gel column (eluting with 1:1 PE/EA) to afford 6-bromotriazolo[1,5-a]pyridine (1.5 g, 7.57 mmol, 89.44% yield) as a yellow solid. ¹H NMR (400 MHz, DMSO-d6) δ 8.91 (s, 1H), 8.08 (s, 1H), 7.62 (d, J=9.2 Hz, 1H), 7.33 (d, J=9.2 Hz, 1H). LCMS (ES, m/z): 198, 200 [M+H]+. Rt 0.576 min.

### Intermediate 134. 1-(1-methyl-4-piperidyl)pyrazol-3-amine

### Step 1. 1-methyl-4-(3-nitropyrazol-1-yl)piperidine

To a solution of 3-nitro-1H-pyrazole (900 mg, 7.96 mmol, 1 eq.), 1-methylpiperidin-4-ol (1.38 g, 11.94 mmol, 1.5 eq.) and triphenylphosphine (4.18 g, 15.92 mmol, 2.0 eq.) in THF (15 mL) was added DIAD (3.22 g, 15.92 mmol, 2 eq.) dropwise. The resulting mixture was stirred for 16 h at room temperature under the N₂ atmosphere. The reaction was monitored by LCMS. Then water (50 mL) was added. The resulting mixture was extracted with EA (50 mL×3). The combined organic layer was dried over anhydrous sodium sulfate. After filtration, the filtrate was concentrated under reduced pressure. The residue was purified by silica gel column chromatography (eluting with 1:1 EA/PE) to afford 1-methyl-4-(3-nitropyrazol-1-yl)piperidine (700 mg, 3.33 mmol, 42% yield) as a yellow solid. LCMS (ES, m/z): 211 [M+H]+, Rt 0.363 min.

### Step 2. 1-(1-methyl-4-piperidyl)pyrazol-3-amine (Intermediate 134)

To a stirred solution of 1-methyl-4-(3-nitropyrazol-1-yl)piperidine (700.00 mg, 3.33 mmol, 1 eq.) in MeOH (10 mL) was added Pd/C (70 mg, 10 wt %) under N₂ atmosphere. The resulting mixture was stirred for 1 h at room temperature under the H₂ atmosphere. The reaction was monitored by LCMS. The resulting mixture was filtered and filter cake was washed with MeOH (3×10 mL). The filtrate was concentrated under reduced pressure to afford 1-(1-methyl-4-piperidyl)pyrazol-3-amine (500 mg, crude) as a yellow solid. LCMS (ES, m/z): 181 [M+H]⁺, Rt 0.432 min.

¹H NMR (400 MHz, Chloroform-d) δ 7.30-7.26 (m, 1H), 5.55 (d, J=2.0 Hz, 1H), 3.98-3.88 (m, 1H), 3.47 (s, 3H), 3.04-2.98 (m, 2H), 2.28-2.20 (m, 2H), 2.19-2.08 (m, 2H), 1.97-1.87 (m, 2H).

TABLE 9
The following intermediates were prepared in an analogous manner as
intermediate 134. Absolute stereochemistry of the following intermediates was arbitrarily
assigned.

| Structure/Intermediate # | LCMS |
|---|---|
| 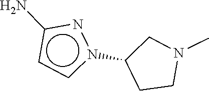 | LCMS (ES, m/z): 167 [M + H]+, Rt 0.503 min. |
| Intermediate 127 | |
| 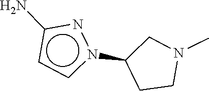 | LCMS (ES, m/z): 167 [M + H]+, Rt 0.559 min. |
| Intermediate 128 | |
| 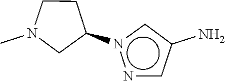 | LCMS (ES, m/z): 167 [M + H]+; RT: 0.372 min. |
| Intermediate 129 | |
| 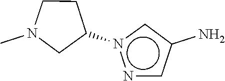 | LCMS (ES, m/z): 167[M + H]+, Rt 0.146 min. |
| Intermediate 130 | |
| 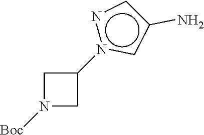 | LCMS (ES, m/z): 239 [M + H]+, Rt 0.545 min. |
| Intermediate 131 | |
| 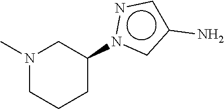 | LCMS (ES, m/z): 181[M + H]+, Rt 0.155 min. |
| Intermediate 132 | |
| 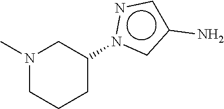 | LCMS (ES, m/z): 181 [M + H]+; RT: 0.128 min. |
| Intermediate 133 | |
| 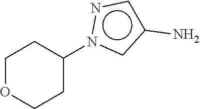 | LCMS (ES, m/z): 168 [M + H]+. Rt 0.433 min. |
| Intermediate 139 | |

### Intermediate 135. tert-butyl 4-(4-aminotriazol-1-yl)piperidine-1-carboxylate

### Step 1. tert-butyl 4-(4-nitrotriazol-1-yl)piperidine-1-carboxylate

Into a 250 mL flask was added 4-nitro-1H-triazole (3 g, 26.30 mmol, 1 eq.), tert-butyl 4-methylsulfonyloxypiperidine-1-carboxylate (11.02 g, 39.45 mmol, 1.5 eq.), K₂CO₃ (10.90 g, 78.90 mmol, 3 eq.) and DMF (50 mL). The mixture was stirred at 80° C. for 16 h. The reaction was completed according to LCMS. Then water (150 mL) was added. The mixture was extracted with EA (50 mL×3). The organic layers were combined, washed with brine (150 mL), dried over Na₂SO₄, filtered and concentrated. The residue was purified by silica gel column chromatography (eluting with 1:1 EA/PE) to afford tert-butyl 4-(4-nitrotriazol-1-yl)piperidine-1-carboxylate (2.2 g, 10.42 mmol, 40% yield) as a yellow solid. LCMS (ES, m/z): 242 [M+H-56]⁺, Rt 0.918 min.

### Step 2. tert-butyl 4-(4-aminotriazol-1-yl)piperidine-1-carboxylate (Intermediate 135)

A mixture of tert-butyl 4-(4-nitrotriazol-1-yl)piperidine-1-carboxylate (500 mg, 1.68 mmol, 1 eq.) and Pd/C (50 mg, 10 wt %) in MeOH (10 mL) was stirred for 2 h at room temperature under H₂ atmosphere. The reaction was monitored by LC-MS. The solids were filtered out and washed with MeOH (5 mL×3). The filtrate was concentrated under reduced pressure. The residue was purified by silica gel column chromatography (eluting with 1:1 EA/PE) to afford tert-butyl 4-(4-aminotriazol-1-yl)piperidine-1-carboxylate (350 mg, 1.31 mmol, 78% yield) as a white solid. HNMR (300 MHz, DMSO-d6) δ 7.18 (s, 1H), 4.69 (br, 2H), 4.58-4.43 (m, 1H), 4.12-3.96 (m, 2H), 3.04-2.79 (m, 2H), 2.09-1.93 (m, 2H), 1.87-1.71 (m, 2H), 1.43 (s, 9H). LCMS (ES, m/z): 268 [M+H]⁺, Rt 0.455 min.

### Intermediate 136. tert-butyl 4-(5-aminoisoxazol-3-yl)piperidine-1-carboxylate

### Step 1. tert-butyl 4-(2-cyanoacetyl)piperidine-1-carboxylate

Into a mixture of acetonitrile (214.67 μL, 4.11 mmol, 1 eq.) in THF (8.0 mL) was added n-BuLi (2.5 M in THF, 1.64 mL, 1 eq.) dropwise at −78° C. under nitrogen atmosphere. The mixture was stirred at −78° C. for 0.5 h. To the above 1-(tert-butyl) 4-methyl piperidine-1,4-dicarboxylate (1 g, 4.11 mmol, 1 eq.) was added slowly. The mixture was stirred at −78° C. for 0.5 h and then warmed to room temperature for another 2 h. The reaction was monitored by LC-MS. To the above was added H₂O (50 mL). The resulting mixture was extracted with EA (3×50 mL). The combined organic layers were washed with brine (100 mL), dried over anhydrous Na₂SO₄. After filtration, the filtrate was concentrated under reduced pressure. The residue was purified by silica gel column chromatography (eluting with 1:2 EA/PE) to afford tert-butyl 4-(2-cyanoacetyl)piperidine-1-carboxylate (0.6 g, 2.38 mmol, 58% yield) as a yellow solid. LCMS (ES, m/z): 197 [M+H-56]⁺, Rt 0.542 min.

¹H NMR (300 MHz, Chloroform-d) δ 3.52 (s, 2H), 2.89-2.65 (m, 3H), 1.94-1.82 (m, 2H), 1.67-1.48 (m, 3H), 1.46 (s, 9H), 1.30-1.22 (m, 1H).

### Step 2. tert-butyl 4-(5-aminoisoxazol-3-yl)piperidine-1-carboxylate (Intermediate 136)

A mixture of tert-butyl 4-(2-cyanoacetyl)piperidine-1-carboxylate (500 mg, 1.98 mmol, 1.0 eq.), hydroxylamine hydrochloride (206.57 mg, 2.97 mmol, 1.5 eq.) and sodium acetate (487.70 mg, 5.95 mmol, 3.0 eq.) in MeOH (10 mL) was stirred at room temperature for 24 h. The reaction was monitored by LC-MS. To the above was added H₂O (50 mL). The resulting mixture was extracted with EA (3×50 mL). The combined organic layers were washed with brine (100 mL), dried over anhydrous Na₂SO₄. After filtration, the filtrate was concentrated under reduced pressure. The residue was purified by silica gel column chromatography (eluting with 2:1 EA/PE) to afford tert-butyl 4-(5-aminoisoxazol-3-yl)piperidine-1-carboxylate (420 mg, 1.57 mmol, 79% yield) as a yellow solid. LCMS (ES, m/z): 212 [M+H-56]⁺, Rt 0.842 min.

### Intermediate 137. tert-butyl 4-(4-amino-1H-imidazol-1-yl)piperidine-1-carboxylate

Prepared in an analogous manner as intermediate 135.

¹H NMR (400 MHz, DMSO-d6) δ 7.06 (s, 1H), 6.91 (s, 1H), 4.16-4.08 (m, 1H), 4.01-3.97 (m, 2H), 3.81-3.74 (m, 2H), 1.93-1.88 (m, 2H), 1.72-1.62 (m, 2H), 1.41 (s, 9H). LCMS (ES, m/z): 267 [M+H]⁺, Rt 0.868 min.

### Intermediate 138. 2-(1-Methylpiperidin-4-yl)thiazol-5-amine

### Step 1. tert-butyl (2-(1-Methyl-1,2,3,6-tetrahydropyridin-4-yl)thiazol-5-yl)carbamate

To a solution of tert-butyl N-(2-bromothiazol-5-yl)carbamate (1 g, 3.58 mmol, 1 eq.) and tert-butyl 4-(4,4,5,5-tetramethyl-1,3,2-dioxaborolan-2-yl)-3,6-dihydro-2H-pyridine-1-carboxylate (1.66 g, 5.37 mmol, 1.5 eq.) in 1,4-dioxane (15 mL) and water (3 mL) were added 1,1′-bis(diphenylphosphino)ferrocene-palladium(II)dichloride (262 mg, 358.23 μmol, 0.1 eq.) and potassium carbonate (1.5 g, 10.75 mmol, 3 eq.). The mixture was stirred for 16 h at 100° C. under a nitrogen atmosphere. The reaction was monitored by LCMS. The reaction was cooled down to room temperature. The resulting mixture was filtered and the filterate was concentrated under reduced pressure. The residue was purified by silica gel column chromatography (eluting with 3:1 PE/EA) to afford tert-butyl N-[2-(1-methyl-3,6-dihydro-2H-pyridin-4-yl)thiazol-5-yl]carbamate (750 mg, 2.31 mmol, 64% yield) as a yellow solid. LCMS (ES, m/z): 296 [M+H]⁺, Rt 0.650 min.

### Step 2. tert-butyl (2-(1-methylpiperidin-4-yl)thiazol-5-yl)carbamate

To a solution of tert-butyl N-[2-(1-methyl-3,6-dihydro-2H-pyridin-4-yl)thiazol-5-yl]carbamate (730 mg, 2.47 mmol, 1 eq.) in ethanol (10 mL) was added Pd/C (150 mg, 10 wt %) under N₂ atmosphere. The mixture was hydrogenated at room temperature for 5 h under H₂ atmosphere using a hydrogen balloon. The reaction was monitored by LCMS. The solids were filtered through a Celite pad and washed with ethanol (2×5 mL). The filtrate was concentrated under reduced pressure to give tert-butyl N-[2-(1-methyl-4-piperidyl)thiazol-5-yl]carbamate (650 mg, crude) as a yellow solid. LCMS (ES, m/z): 298 [M+H]⁺, Rt 0.642 min.

### Step 3. 2-(1-methylpiperidin-4-yl)thiazol-5-amine (Intermediate 138)

To a solution of tert-butyl N-[2-(1-methyl-4-piperidyl)thiazol-5-yl]carbamate (460 mg, 1.54 mmol, 1 eq.) in ethanol (8 mL) was added HCl (4M in dioxane, 4 mL). The mixture was stirred for 2 h at room temperature. The reaction was monitored by LCMS. The mixture was concentrated under reduced pressure. The crude product was purified by reverse phase chromatography (Column: C18; Mobile phase, A: water (containing 10 mmol/L NH₄HCO₃) and B: ACN (5% to 40% over 35 min); Detector, UV 254 nm) to tert-butyl N-[2-(1-methyl-4-piperidyl)thiazol-5-yl]carbamate (180 mg, 913 μmol, 59% yield) as a yellow solid. LCMS (ES, m/z): 198 [M+H]⁺, Rt 0.775 min. ¹H NMR (400 MHz, DMSO-d6) δ 6.56 (s, 1H), 5.34 (br, 2H), 2.79-2.74 (m, 2H), 2.67-2.60 (m, 1H), 2.15 (s, 3H), 1.98-1.86 (m, 4H), 1.64-1.54 (m, 2H).

### Intermediate 142. tert-butyl 4-(4-bromo-2-oxo-1-pyridyl)piperidine-1-carboxylate

To a stirred mixture of 4-bromo-1H-pyridin-2-one (2 g, 11.49 mmol, 1 eq.) and tert-butyl 4-methylsulfonyloxypiperidine-1-carboxylate (6.42 g, 22.99 mmol, 2 eq.) in DMSO (20 mL) was added K₂CO₃ (4.77 g, 34.48 mmol, 3 eq.). The reaction was stirred for 16 h at 80° C. The mixture was allowed to cool down to room temperature and was diluted with H₂O (200 mL). The resulting mixture was extracted with EA (3×150 mL). The combined organic layers were washed with brine (2×200 mL) and dried over anhydrous Na₂SO₄. After filtration, the filtrate was concentrated under reduced pressure. The residue was purified by silica gel column chromatography, eluted with EA/PE (2:1) to afford tert-butyl 4-(4-bromo-2-oxo-1-pyridyl)piperidine-1-carboxylate (400 mg, 1.12 mmol, 10% yield) as a yellow solid. ¹H NMR (300 MHz, DMSO-d6) δ 7.75 (d, J=7.5 Hz, 1H), 6.72 (d, J=2.1 Hz, 1H), 6.49-6.40 (m, 1H), 4.86-4.69 (m, 1H), 4.14-3.97 (m, 2H), 2.99-2.73 (m, 2H), 1.77-1.60 (m, 4H), 1.42 (s, 9H). LCMS (ES, m/z): 357, 359 [M+H]⁺, Rt 0.718 min.

### Intermediate 144. 4-bromo-1-tetrahydropyran-4-yl-pyridin-2-one

To a stirred mixture of 4-bromo-1H-pyridin-2-one (500 mg, 2.87 mmol, 1 eq.) and tetrahydropyran-4-yl methanesulfonate (1.04 g, 5.75 mmol, 2 eq.) in DMSO (10 mL) were added K₂CO₃ (1.19 g, 8.62 mmol, 3 eq.) at room temperature. The resulting mixture was stirred for 16 h at 80° C. The mixture was allowed to cool down to room temperature and diluted with H₂O (200 mL). The resulting mixture was extracted with EA (3×150 mL). The combined organic layers were washed with brine (2×200 mL) and dried over anhydrous Na₂SO₄. After filtration, the filtrate was concentrated under reduced pressure. The residue was purified by reverse phase chromatography (Column: C18; Mobile phase A: water containing 10 mmol/L NH₄HCO₃ and B: ACN (30% B to 40% B over 10 min); Detector: UV 254 nm) to afford 4-bromo-1-tetrahydropyran-4-yl-pyridin-2-one (200 mg, 775.19 μmol, 27% yield) as a white solid. ¹H NMR (400 MHz, DMSO-d6) δ 7.75 (d, J=7.6 Hz, 1H), 6.73 (d, J=2.4 Hz, 1H), 6.55-6.44 (m, 1H), 4.90-4.78 (m, 1H), 4.01-3.93 (m, 2H), 3.51-3.40 (m, 2H), 1.94-1.79 (m, 2H), 1.73-1.64 (m, 2H). LCMS (ES, m/z): 258, 260 [M+H]⁺, Rt 0.559 min.

TABLE 10
The following intermediates were prepared in an analogous manner as
Intermediates 142 and 144.

| Structure/Intermediate # | LCMS |
|---|---|
| 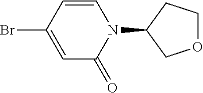 [SMILES: O=c1cc(Br)ccn1[C@H]1CCOC1] | LCMS (ES, m/z): 244, 246 [M + H]⁺, Rt 0.833 min. |
| Intermediate 145 | |
| 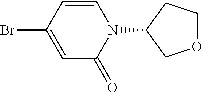 [SMILES: O=c1cc(Br)ccn1[C@@H]1CCOC1] | LCMS (ES, m/z): 244, 246 [M + H]⁺, Rt 0.557 min. |
| Intermediate 146 | |
| 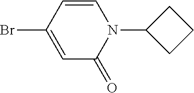 [SMILES: O=c1cc(Br)ccn1C1CCC1] | LCMS (ES, m/z): 228, 230 [M + H]⁺, Rt 0.548 min. |
| Intermediate 286 | |
| 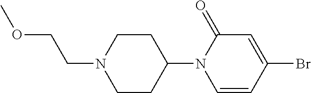 [SMILES: COCCN1CCC(n2ccc(Br)cc2=O)CC1] | LCMS (ES, m/z): 315, 317 [M + H]⁺, Rt 0.923 min. |
| Intermediate 287 | |
| 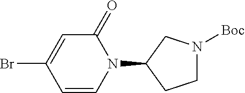 [SMILES: CC(C)(C)OC(=O)N1CC[C@@H](n2ccc(Br)cc2=O)C1] | LCMS (ES, m/z): 343, 345 [M + H]⁺, Rt 0.707 min. |
| Intermediate 288 | |
| 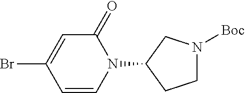 [SMILES: CC(C)(C)OC(=O)N1CC[C@H](n2ccc(Br)cc2=O)C1] | LCMS (ES, m/z): 343, 345 [M + H]⁺, Rt 0.783 min. |
| Intermediate 289 | |

### Intermediate 147. 4-bromo-1-(difluoromethyl)pyridin-2(1H)-one

To a solution of 4-bromo-2-chloropyridine (1.25 g, 6.51 mmol, 1 eq) in CH₃CN (15 mL) was added NaHCO₃ (1.64 g, 19.5 mmol, 3 eq) in portions. The reaction mixture was stirred at 80° C. for 30 minutes. Then a solution of 2,2-difluoro-2-(fluorosulfonyl)acetic acid (3.47 g, 19.5 mmol, 3 eq) in CH₃CN (10 mL) was added over 10 minutes and the reaction mixture was stirred at 80° C. for 2 h. The mixture was cooled to room temperature and diluted with water (100 mL), neutralized with 10% aq. NaHCO₃ and extracted with EA (2×100 mL). The combined organic layers were washed with brine (150 mL), dried over anhydrous Na₂SO₄. After filtrate, the filtration was concentrated under reduced pressure. The residue was purified by silica gel column chromatography, eluted with PE/EA (4:1) to afford 4-bromo-1-(difluoromethyl)pyridin-2(1H)-one (560 mg, 2.29 mmol, 39% yield) as a yellow oil. LCMS (ES, m/z): 224, 226 [M+H]⁺, Rt 0.598 min.

### Intermediate 148. 4-bromo-1-(methyl-d3)pyridin-2(1H)-one

To a stirred solution of 4-bromo-1H-pyridin-2-one (200 mg, 1.15 mmol, 1 eq.) in DMF (2 mL) was added NaH (60% purity, 114.8 mg, 2.87 mmol, 2.5 eq.) in portions at 0° C. under the N₂ atmosphere. The resulting mixture was stirred for 25 min at −5° C. before trideuterio(iodo)methane (199.95 mg, 1.38 mmol, 1.2 eq.) was added dropwise over 5 min at −5° C. The resulting mixture was stirred for 2 h at room temperature, then it was quenched with ice/water (50 mL) at 0° C. and extracted with EA (3×40 mL). The combined organic layers were washed with brine (2×80 mL), dried over anhydrous Na₂SO₄. After filtration, the filtrate was concentrated under reduced pressure. The residue was purified by silica gel column chromatography, eluted with MeOH/DCM (1:10) to afford 4-bromo-1-(methyl-d3)pyridin-2(1H)-one (140 mg, 725.50 μmol, 63% yield) as a white solid. ¹H NMR (400 MHz, DMSO-d6) δ 7.69 (d, J=7.2 Hz, 1H), 6.70 (s, 1H), 6.45 (d, J=7.2 Hz, 1H). LCMS (ES, m/z): 191, 193 [M+H]⁺, Rt 0.495 min.

### Intermediate 149. 4-bromo-1-cyclopropylpyridin-2(1H)-one

To a stirred mixture of 4-bromo-1H-pyridin-2-one (500 mg, 2.87 mmol, 1 eq.) and cyclopropylboronic acid (617.09 mg, 7.18 mmol, 2.5 eq.) in DCE (10 mL) were added 2,2′-bipyridine (448.82 mg, 2.87 mmol, 1 eq.), Cu(OAc)₂ (521.95 mg, 2.87 mmol, 1 eq.) and Na₂CO₃ (913.73 mg, 8.62 mmol, 3 eq.) at room temperature under the N₂ atmosphere. The resulting mixture was stirred for 18 h at 70° C. under the N₂ atmosphere. The reaction was allowed to cool down to room temperature. The resulting mixture was filtered, the filter cake was washed with EA (3×10 mL). The filtrate was concentrated under reduced pressure. The residue was purified by silica gel column chromatography, eluted with EA/PE (1:1) to afford 4-bromo-1-cyclopropylpyridin-2(1H)-one (300 mg, 1.40 mmol, 48% yield) as a light yellow oil. ¹H NMR (300 MHz, DMSO-d₆) δ 7.53 (d, J=7.2 Hz, 1H), 6.68 (d, J=2.1 Hz, 1H), 6.42-6.33 (m, 1H), 3.34-3.21 (m, 1H), 1.07-0.98 (m, 2H), 0.93-0.77 (m, 2H). LCMS (ES, m/z): 214, 216 [M+H]⁺, Rt 0.646 min.

### Intermediate 160. 1-(6-bromo-2-pyridyl)pyrrolidin-2-one

Into a solution of 2,6-dibromopyridine (500 mg, 2.11 mmol, 1 eq.) and pyrrolidin-2-one (179.63 mg, 2.11 mmol, 1 eq.) in dioxane (10 mL) was added Pd₂dba₃ (193.13 mg, 211.07 μmol, 0.1 eq.), BINAP (131.42 mg, 211.07 μmol, 0.1 eq.) and Cs₂CO₃ (1.38 g, 4.22 mmol, 2 eq.). The mixture was stirred for 1 h at 100° C. under the N₂ atmosphere. The reaction was monitored by LC-MS. The mixture was allowed to cool down to room temperature. The solids were filtered out and washed with EA (3×10 mL). The filtrate was concentrated under reduced pressure. The residue was purified by silica gel column chromatography (eluting with 1:5 EA/PE) to afford 1-(6-bromo-2-pyridyl)pyrrolidin-2-one (100 mg, 414.79 μmol, 19.65% yield) as a yellow solid. LCMS (ES, m/z): 241, 243 [M+H]⁺, Rt 0.720 min.

TABLE 11
The following intermediate were prepared in an analogous manner as
intermediate 160.

| Structure/Intermediate # | LCMS |
|---|---|
| 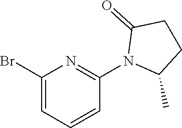 | LCMS (ES, m/z): 255, 257 [M + H]⁺, Rt 0.683 min. |
| Intermediate 159 | |

### Intermediate 161. (6-bromopyridin-2-yl)dimethylphosphine oxide

Into a mixture of 2,6-dibromopyridine (1 g, 4.22 mmol, 1 eq.) and dimethylphosphine oxide (988.42 mg, 12.66 mmol, 3 eq.) in ACN (12 mL) were added TEA (1.50 g, 14.77 mmol, 2.06 mL, 3.5 eq.) and Pd(PPh₃)₄ (243.90 mg, 211.07 μmol, 0.05 eq.). The reaction was stirred for 13 h at 90° C. under the N₂ atmosphere. The mixture was allowed to cool down to room temperature and concentrated under reduced pressure. The residue was purified by silica gel column chromatography (eluting with 1:1 EA/PE) to afford (6-bromopyridin-2-yl)dimethylphosphine oxide (520 mg, 2.23 mmol, 52% yield) as a white solid. LCMS (ES, m/z): 234, 236 [M+H]⁺, Rt 0.513 min.

### Intermediate 163. 1-(6-bromopyridin-2-yl)-5,5-dimethylpyrrolidin-2-one

To a stirred mixture of 2,6-dibromopyridine (1 g, 4.22 mmol, 1 eq) and 5,5-dimethylpyrrolidin-2-one (382.14 mg, 3.38 mmol, 0.8 eq.) in 1,4-dioxane (10 mL) was added CuI (803.95 mg, 4.22 mmol, 1 eq.), DMEDA (372.11 mg, 4.22 mmol, 1 eq) and K₂CO₃ (1.75 g, 12.66 mmol, 3 eq.). The reaction was stirred for 4 h at 100° C. under the N₂ atmosphere and the mixture was allowed to cool down to room temperature. The solids were filtered out and washed with EA (3×5 mL). The filtrate was dried over anhydrous Na₂SO₄ and concentrated. The residue was purified by silica gel column chromatography (eluting with 1:2 EA/PE) to afford 1-(6-bromopyridin-2-yl)-5,5-dimethylpyrrolidin-2-one (400 mg, 1.49 mmol, 35% yield) as a light-yellow oil. LCMS (ES, m/z): 269, 271 [M+H]⁺, Rt 0.726 min.

### Intermediate 167. 2-bromo-6-(1-((tert-butyldimethylsilyl)oxy)cyclopropyl)pyridine

### Step 1. 2-bromo-6-(1-((tert-butyldimethylsilyl)oxy)vinyl)pyridine

To a solution of 1-(6-bromo-2-pyridyl)ethanone (1 g, 5.00 mmol, 1 eq.) and triethylamine (1.52 g, 15.00 mmol, 3 eq.) in DCM (15 mL) was added TBSOTf (1.59 g, 6.00 mmol, 1.2 eq.) dropwise at 0° C. The resulting mixture was stirred for 1 h at room temperature under the N₂ atmosphere. The resulting mixture was diluted with H₂O (100 mL). The resulting mixture was extracted with DCM (3×100 mL). The combined organic layers were washed with brine (150 mL), dried over anhydrous MgSO₄. After filtration, the filtrate was concentrated under reduced pressure. The residue was purified by silica gel column chromatography, eluted with EA/PE (1:3) to afford 2-bromo-6-(1-((tert-butyldimethylsilyl)oxy)vinyl)pyridine (1.3 g, 4.06 mmol, 82% yield) as a colorless oil. LCMS (ES, m/z): 314, 316 [M+H]⁺, Rt 1.383 min.

### Step 2. 2-bromo-6-(1-((tert-butyldimethylsilyl)oxy)cyclopropyl)pyridine

To a solution of diethylzinc (14.75 g, 119.42 mmol, 30 eq.) in DCM (30 mL) was added chloro(iodo)methane (4.49 g, 25.45 mmol, 6.4 eq.) in DCM (6 mL) dropwise at 0° C. under N₂ atmosphere. The mixture was stirred at 0° C. for 15 min. To the above was added 2-bromo-6-(1-((tert-butyldimethylsilyl)oxy)vinyl)pyridine (1.25 g, 3.98 mmol, 1 eq.) in DCM (18 mL) at 0° C. The reaction mixture was stirred at 0° C. for 2 h. The resulting mixture was diluted with H₂O (100 mL). The resulting mixture was extracted with DCM (3×100 mL). The combined organic layers were washed with brine (200 mL), dried over anhydrous Na₂SO₄. After filtration, the filtrate was concentrated under reduced pressure. The residue was purified by silica gel column chromatography, eluted with EA/PE (1:4) to afford 2-bromo-6-(1-((tert-butyldimethylsilyl)oxy)cyclopropyl)pyridine (340 mg, 602.60 μmol, 15% yield) as a colorless oil. ¹H NMR (300 MHz, d6-DMSO) δ 7.74 (t, J=7.8 Hz, 1H), 7.56 (d, J=7.8 Hz, 1H), 7.42 (d, J=8.1 Hz, 1H), 1.29-1.18 (m, 4H), 0.91 (s, 9H), 0.08 (s, 6H). LCMS (ES, m/z): 328, 330 [M+H]⁺, Rt 1.258 min.

### Intermediate 168. 3-(6-bromo-2-pyridyl)oxetan-3-ol

To a stirred solution of 2,6-dibromopyridine (2 g, 8.44 mmol, 1 eq.) in THF (50 mL) was added n-BuLi (1.3M/n-hexane, 7.14 mL, 9.29 mmol, 1.1 eq.) dropwise at −78° C. under N₂ atmosphere. The resulting mixture was stirred for 30 min at −78° C. To the above solution was added oxetan-3-one (730.08 mg, 10.13 mmol, 1.2 eq) dropwise. The resulting mixture was stirred for 2 h at −78° C. The reaction was monitored by LCMS. The reaction mixture was quenched with saturated NH₄Cl (aq.) at −78° C. The combined organic layers were extracted with EA (3×100 mL), dried over anhydrous Na₂SO₄. After filtration, the filtrate was concentrated under reduced pressure. The residue was purified by silica gel column chromatography, eluted with EA/PE (1:4) to afford 3-(6-bromo-2-pyridyl)oxetan-3-ol (1.4 g, 6.09 mmol, 72% yield) as a white solid. LCMS (ES, m/z): 230, 232 [M+H]⁺, Rt 0.612 min.

### Intermediate 169. 2-chloro-7-ethyl-6,7-dihydro-5H-cyclopenta[b]pyridin-7-ol

To a solution of 2-chloro-5,6-dihydro-7H-cyclopenta[b]pyridin-7-one (6 g, 35.80 mmol, 1 eq.) in THF (200 mL) was added ethylmagnesium bromide (1 M in THF, 53.70 mL, 1.5 eq.) dropwise at −5° C. The mixture was stirred for 2 h at this temperature. The reaction was monitored by LCMS. Then water (300 mL) was added. The mixture was extracted with EA (3×100 mL). The organic layers were combined, washed by brine (200 mL), dried over Na₂SO₄, filtered and concentrated. The residue was purified by silica gel column chromatography (eluting with 1:5 PE/EA) to afford 2-chloro-7-ethyl-6,7-dihydro-5H-cyclopenta[b]pyridin-7-ol (4.2 g, 21.25 mmol, 59% yield) as a white solid. LCMS (ES, m/z): 198, 200 [M+H]⁺, Rt 0.688 min.

### Intermediate 170. ((3-bromophenyl)imino)dimethyl-l6-sulfanone

To a stirred mixture of 1-bromo-3-iodo-benzene (1 g, 3.53 mmol, 1 eq) and iminodimethyl-l6-sulfanone (493.88 mg, 5.30 mmol, 1.5 eq.) in 1,4-dioxane (5 mL) was added Pd₂dba₃ (80.86 mg, 88.37 μmol, 0.025 eq.), Xantphos (152.70 mg, 265.11 μmol, 0.075 eq.) and Cs₂CO₃ (1.73 g, 5.30 mmol, 1.5 eq.). The mixture was stirred for 2 h at 100° C. under the N₂ atmosphere. The reaction was monitored by LC-MS. The mixture was allowed to cool down to room temperature. The solids were filtered out and washed with EA (2×10 mL). The filtrate was concentrated under reduced pressure. The residue was purified by silica gel column chromatography (eluting with 1.5:1 EA/PE) to afford ((3-bromophenyl)imino)dimethyl-l6-sulfanone (0.5 g, 2.01 mmol, 57% yield) as yellow oil. LCMS (ES, m/z): 248, 250 [M+H]⁺, Rt 0.739 min.

### Intermediates 171 and 172. tert-butyl (R)-(1-(6-bromopyridin-2-yl)ethyl)(methyl)carbamate and tert-butyl (S)-(1-(6-bromopyridin-2-yl)ethyl)(methyl)carbamate

### Step 1. 1-(6-bromopyridin-2-yl)ethan-1-amine

To a mixture of 1-(6-bromopyridin-2-yl)ethan-1-one (2 g, 10.00 mmol, 1 eq.), NH₄OAc (1.54 g, 20.00 mmol, 2 eq.) in MeOH (30 mL) was added NaBH₃CN (1.24 g, 20.00 mmol, 2 eq.) in portions at 0° C. The mixture was stirred for 18 h at room temperature. The mixture was diluted with water (150 mL). The resulting mixture was washed with EA (3×100 mL). The combined organic phase was dried over anhydrous Na₂SO₄. The filtrate was concentrated under reduced pressure and residue was purified by silica gel column chromatography, eluted with PE/EA (1:1) to afford 1-(6-bromopyridin-2-yl)ethan-1-amine (1 g, 4.95 mmol, 50% yield) as a white solid. LCMS (ES, m/z): 201, 203 [M+H]⁺, Rt 0.430 min.

### Step 2. tert-butyl (1-(6-bromopyridin-2-yl)ethyl)carbamate

The mixture of 1-(6-bromopyridin-2-yl)ethan-1-amine (1.5 g, 7.46 mmol, 1 eq.), TEA (2.26 g, 22.38 mmol, 3 eq.) and Boc₂O (1.95 g, 8.95 mmol, 1.2 eq.) in THF (15 mL) was stirred for 2 h room temperature. The mixture was diluted with water (150 mL) and extracted with EA (3×100 mL). The combined organic phase was dried over anhydrous Na₂SO₄. After filtration, the filtrate was concentrated under reduced pressure. The residue was purified by silica gel column chromatography, eluted with PE/EA (2:1) to afford tert-butyl (1-(6-bromopyridin-2-yl)ethyl)carbamate (1.0 g, 3.32 mmol, 45% yield) as white solid. LCMS (ES, m/z): 301, 303 [M+H]⁺, Rt 0.850 min.

### Step 3. tert-butyl (1-(6-bromopyridin-2-yl)ethyl)(methyl)carbamate

To a stirred solution of tert-butyl (1-(6-bromopyridin-2-yl)ethyl)carbamate (500 mg, 1.66 mmol, 1 eq.) in DMF (10 mL) was added NaH (199.20 mg, 4.98 mmol, 60% purity, 3 eq.) in portions at 0° C. The mixture was stirred for 0.5 h at 25° C. To the above mixture was added Mel (282.77 mg, 1.99 mmol, 1.2 eq.) dropwise at 0° C. The mixture was stirred for an additional 2 h at 25° C. The reaction was monitored with LCMS. The mixture was diluted with water (100 mL) and extracted with EA (3×80 mL). The combined organic phase was dried over anhydrous Na₂SO₄. After filtration, the filtrate was concentrated under reduced pressure. The residue was purified by silica gel column chromatography, eluted with PE/EA (2:1) to afford to afford tert-butyl (1-(6-bromopyridin-2-yl)ethyl)(methyl)carbamate (300 mg, 0.95 mmol, 57% yield) as a yellow oil. LCMS (ES, m/z): 315, 317 [M+H]⁺, Rt 0.844 min.

### Step 4. tert-butyl (R)-(1-(6-bromopyridin-2-yl)ethyl)(methyl)carbamate and tert-butyl (S)-(1-(6-bromopyridin-2-yl)ethyl)(methyl)carbamate

Tert-butyl (1-(6-bromopyridin-2-yl)ethyl)(methyl)carbamate (300 mg, 0.95 mmol, 1 eq.) was separated by Chiral-HPLC (Column: NB_ASA CHIRAL ART Cellulose-SC(IC), 5*25 cm/10 μm; Mobile Phase A: Hex (0.2% DEA), Mobile Phase B: IPA; Flow rate: 20 mL/min; Gradient: 1% B in 20 min; 220/254 nm; RT1(min): 14.1; RT2(min): 15.8) to afford tert-butyl (S)-(1-(6-bromopyridin-2-yl)ethyl)(methyl)carbamate (150 mg, 0.48 mmol, 50% yield) as a yellow oil and tert-butyl (R)-(1-(6-bromopyridin-2-yl)ethyl)(methyl)carbamate (130 mg, 0.41 mmol, 43% yield) as a yellow oil. LCMS (ES, m/z): 315, 317 [M+H]⁺, Rt 0.844 min. Absolute stereochemistry of title compounds were assigned arbitrarily upon chiral separation.

### Intermediate 175. tert-butyl 4-(6-bromo-2-pyridyl)piperidine-1-carboxylate

A mixture of 2,6-dibromopyridine (500 mg, 2.11 mmol, 1 eq), potassium (1-(tert-butoxycarbonyl)piperidin-4-yl)trifluoroborate (614.54 mg, 2.11 mmol, 1 eq), (Ir[dF(CF3)ppy]₂(dtbpy))PF₆ (23.68 mg, 21.11 umol, 0.01 eq.), NiCl₂(DME) (46.43 mg, 211.07 umol, 0.1 eq.), dtbbpy (84.85 mg, 316.60 umol, 0.15 eq.) and Cs₂CO₃ (1.38 g, 4.22 mmol, 2 eq.) in dioxane (40 mL) was irradiated with blue light (450 nm) for 1 h at 20° C. under nitrogen atmosphere. The reaction was monitored by LCMS. The reaction was diluted with brine (100 mL) and was extracted with ethyl acetate (3×80 mL). The combined organic layers were dried over anhydrous Na₂SO₄. After filtration, the filtrate was concentrated under reduced pressure. The residue was purified by silica gel column chromatography (eluting with 1:4 EA/PE) to afford tert-butyl 4-(6-bromo-2-pyridyl)piperidine-1-carboxylate (350 mg, 1.03 mmol, 49% yield) as a white solid. ¹H NMR (300 MHz, DMSO-d6) δ 7.63 (t, J=7.8 Hz, 1H), 7.42 (d, J=8.1 Hz, 1H), 7.29 (d, J=7.8 Hz, 1H), 4.28-4.15 (m, 2H), 3.00-2.81 (m, 3H), 1.96-1.81 (m, 2H), 1.76-1.57 (m, 2H), 1.50 (s, 9H). LCMS (ES, m/z): 341, 343 [M+H]⁺, Rt 0.807 min.

### Intermediate 176. 1-(6-bromo-2-pyridyl)-4-methyl-piperazine

A solution of 2,6-dibromopyridine (200 mg, 844.27 umol, 1 eq.), 1-methylpiperazine (84.56 mg, 844.27 umol, 1 eq.), BINAP G2-Pd (78.69 mg, 84.43 umol, 0.1 eq.), BINAP (52.57 mg, 84.43 umol, 0.1 eq.) and Cs₂CO₃ (550.46 mg, 1.69 mmol) in dioxane (2 mL) was stirred for 2 h at 100° C. The reaction was monitored by LC-MS. The mixture was allowed to cool down to room temperature. The resulting mixture was diluted with brine (50 mL) and was extracted with ethyl acetate (3×50 mL). The combined organic layers were dried over anhydrous Na₂SO₄. After filtration, the filtrate was concentrated under reduced pressure. The residue was purified by silica gel column chromatography (eluting with 20:1 DCM/MeOH) to afford 1-(6-bromo-2-pyridyl)-4-methyl-piperazine (140 mg, 546.88 umol, 65% yield) as yellow oil.

¹H NMR (300 MHz, DMSO-d6) δ 7.48-7.40 (m, 1H), 7.42 (d, J=7.8 Hz, 2H), 3.53-3.42 (m, 4H), 2.43-2.35 (m, 4H), 2.23 (s, 3H). LCMS (ES, m/z): 256, 258 [M+H]⁺, Rt 0.487 min.

### Intermediate 177 and Intermediate 178. tert-butyl (R)-3-(6-bromopyridin-2-yl)pyrrolidine-1-carboxylate and tert-butyl (S)-3-(6-bromopyridin-2-yl)pyrrolidine-1-carboxylate

### Step 1. tert-butyl 3-(6-bromo-2-pyridyl)pyrrolidine-1-carboxylate

A mixture of 2,6-dibromopyridine (500 mg, 2.11 mmol, 1 eq.), tert-butyl 3-bromopyrrolidine-1-carboxylate (791.92 mg, 3.17, mmol, 1.5 eq.), (Ir[dF(CF3)ppy]₂(dtbpy))PF₆ (23.68 mg, 21.11 umol, 0.01 eq.), dtbbpy (84.85 mg, 316.60 umol, 0.15 eq.), Cs₂CO₃ (1.38 g, 4.22 mmol, 2 eq.), tris(trimethylsilyl)silane (523.45 mg, 2.11 mmol, 1 eq.) and NiBr₂(DME) (65.01 mg, 211.07 umol, 0.1 eq.) in DMAc (20 mL) was irradiated with blue light (450 nm) for 1 h at 20° C. under nitrogen atmosphere. The reaction was monitored by LC-MS. The resulting mixture was diluted with brine (80 mL) and was extracted with EA (3×50 mL). The combined organic layers were dried over anhydrous Na₂SO₄. After filtration, the filtrate was concentrated under reduced pressure. The residue was purified by silica gel column chromatography (eluting with 1:10 EA/PE) to afford tert-butyl 3-(6-bromo-2-pyridyl)pyrrolidine-1-carboxylate (150 mg, 458.72 umol, 22% yield) as a white solid. LCMS (ES, m/z): 327, 329 [M+H]⁺, Rt 0.803 min.

### Step 2. tert-butyl (R)-3-(6-bromopyridin-2-yl)pyrrolidine-1-carboxylate and tert-butyl (S)-3-(6-bromopyridin-2-yl)pyrrolidine-1-carboxylate

Tert-butyl 3-(6-bromo-2-pyridyl)pyrrolidine-1-carboxylate (1.1 g, 3.36 mmol, 1 eq.) was separated by Chiral-SFC: (Column: Exsil Chiral-NR, 3*25 cm/8 μm; Mobile Phase A: CO₂, Mobile Phase B: IPA:HEX=1:1 (0.1%2M NH₃-MeOH); Flow rate: 100 mL/min; Gradient: isocratic 20% B; Column Temperature(° C.): 35; Back Pressure(bar): 100; 220 nm; RT1(min): 8.72; RT2(min): 10.32) to afford tert-butyl (R)-3-(6-bromopyridin-2-yl)pyrrolidine-1-carboxylate (480 mg, 1.47 mmol, 44% yield) as a white solid and tert-butyl (S)-3-(6-bromopyridin-2-yl)pyrrolidine-1-carboxylate (420 mg, 1.28 mmol, 38% yield) as a white solid. LCMS (ES, m/z): 327, 329 [M+H]⁺, Rt 0.803 min.

Absolute stereochemistry of title compounds were assigned arbitrarily upon chiral separation.

### Intermediate 173 and Intermediate 174. tert-butyl (R)-2-(6-bromopyridin-2-yl)pyrrolidine-1-carboxylate and tert-butyl (S)-2-(6-bromopyridin-2-yl)pyrrolidine-1-carboxylate

The titled compounds were prepared in a similar manner as Intermediates 177 and 178, except with tert-butyl 2-bromopyrrolidine-1-carboxylate in place of tert-butyl 3-bromopyrrolidine-1-carboxylate. LCMS (ES, m/z): 327, 329 [M+H]⁺, Rt 0.858 min.

### Intermediate 179 and Intermediate 180. tert-butyl (S)-(2-chloro-6,7-dihydro-5H-cyclopenta[b]pyridin-7-yl)(methyl)carbamate and tert-butyl (R)-(2-chloro-6,7-dihydro-5H-cyclopenta[b]pyridin-7-yl)(methyl)carbamate

### Step 1. 2-chloro-N-methyl-6,7-dihydro-5H-cyclopenta[b]pyridin-7-amine

To a solution of 2-chloro-5,6-dihydrocyclopenta[b]pyridin-7-one (1 g, 5.97 mmol, 1 eq.) and methylamine (2M in THF, 9 mL, 17.90 mmol, 3 eq.) in DCM (15 mL) was added AcOH (525.00 mg, 8.74 mmol, 1.5 eq.) at room temperature for 2 h under the N₂ atmosphere. Then sodium triacetoxyborohydride (3.79 g, 17.90 mmol, 3 eq.) was added and stirred overnight at room temperature. The reaction was quenched with water (100 mL) and extracted with DCM (3×80 mL). The combined organic layers were washed with brine (100 mL), dried over anhydrous Na₂SO₄. After filtration, the filtrate was concentrated under reduced pressure. The residue was purified by silica gel column chromatography, eluted with EA/PE (1:1) to afford 2-chloro-N-methyl-6,7-dihydro-5H-cyclopenta[b]pyridin-7-amine (580 mg, 3.19 mmol, 54% yield) as a yellow oil. LCMS (ES, m/z): 183 [M+H]⁺, Rt 0.464 min.

### Step 2. tert-butyl (2-chloro-6,7-dihydro-5H-cyclopenta[b]pyridin-7-yl)(methyl)carbamate

In a solution of 2-chloro-N-methyl-6,7-dihydro-5H-cyclopenta[b]pyridin-7-amine (580 mg, 3.18 mmol, 1 eq.) and TEA (963.98 mg, 9.53 mmol, 3 eq.) in DCM (10 mL) was added Boc₂O (900.96 mg, 4.13 mmol, 1.3 eq.) in DCM (0.5 mL) at 0° C. Then the mixture was stirred for 2 h at room temperature. The reaction was quenched with water (60 mL) and extracted with DCM (3×40 mL). The combined organic layers were washed with brine (100 mL), dried over anhydrous Na₂SO₄. After filtration, the filtrate was concentrated under reduced pressure. The residue was purified by silica gel column chromatography, eluted with EA/PE (1:3) to afford tert-butyl (2-chloro-6,7-dihydro-5H-cyclopenta[b]pyridin-7-yl)(methyl)carbamate (785 mg, 2.78 mmol, 87% yield) as a yellow oil. LCMS (ES, m/z): 283 [M+H]⁺, Rt 1.084 min.

### Step 3. tert-butyl (S)-(2-chloro-6,7-dihydro-5H-cyclopenta[b]pyridin-7-yl)(methyl)carbamate and tert-butyl (R)-(2-chloro-6,7-dihydro-5H-cyclopenta[b]pyridin-7-yl)(methyl)carbamate

Tert-butyl (2-chloro-6,7-dihydro-5H-cyclopenta[b]pyridin-7-yl)(methyl)carbamate (900 mg, 3.18 mmol, 1 eq.) was separated by SFC: (Column: OptiChiral-C9-5, 3*25 cm/5 μm; Mobile Phase A: CO₂, Mobile Phase B: IPA (0.5% 2M NH₃-MeOH); Flow rate: 70 mL/min; Gradient: isocratic 25% B; Column Temperature(° C.): 35; Back Pressure(bar): 100; Wave Length: 220 nm; RT1(min): 3.9; RT2(min): 5.58) to give tert-butyl (S)-(2-chloro-6,7-dihydro-5H-cyclopenta[b]pyridin-7-yl)(methyl)carbamate (350 mg, 1.24 mmol, 39% yield) and tert-butyl (R)-(2-chloro-6,7-dihydro-5H-cyclopenta[b]pyridin-7-yl)(methyl)carbamate (310 mg, 1.10 mmol, 34% yield) as a colorless oil. LCMS (ES, m/z): 283 [M+H]⁺, Rt 1.084 min.

Absolute stereochemistry of title compounds were assigned arbitrarily upon chiral separation.

### Intermediate 181 and Intermediate 182. tert-butyl (S)-(2-chloro-7-methyl-6,7-dihydro-5H-cyclopenta[b]pyridin-7-yl)carbamate and tert-butyl (R)-(2-chloro-7-methyl-6,7-dihydro-5H-cyclopenta[b]pyridin-7-yl)carbamate

### Step 1. 2-chloro-7-methyl-6,7-dihydro-5H-cyclopenta[b]pyridin-7-ol

To a stirred solution of 2-chloro-5,6-dihydrocyclopenta[b]pyridin-7-one (5 g, 29.83 mmol, 1 eq) in THF (50 mL) was added methylmagnesium chloride (11.16 g, 149.17 mmol, 5 eq) dropwise at 0° C. under N₂ atmosphere. The resulting mixture was stirred for 16 h at 25° C. under the N₂ atmosphere. The reaction mixture was poured into ice water (200 mL) and stirred for 30 min. The resulting mixture was extracted with EtOAc (2×150 mL). The combined organic layers were washed with brine (200 mL) and dried over anhydrous Na₂SO₄. After filtration, the filtrate was concentrated under reduced pressure. The residue was purified by silica gel column chromatography, eluted with PE/EA (1:2) to afford 2-chloro-7-methyl-6,7-dihydro-5H-cyclopenta[b]pyridin-7-ol (2.3 g, 11.27 mmol, 38% yield) as a green solid. ¹H NMR (300 MHz, DMSO-d6) δ 7.72 (d, J=7.8 Hz, 1H), 7.32 (d, J=7.8 Hz, 1H), 5.24 (s, 1H), 2.96-2.68 (m, 2H), 2.10 (t, J=6.9 Hz, 2H), 1.43 (s, 3H). LCMS (ES, m/z): 184, 186 [M+H]⁺, Rt 1.087 min.

### Step 2. 7-azido-2-chloro-7-methyl-6,7-dihydro-5H-cyclopenta[b]pyridine

To a stirred solution of boron trifluoride etherate (278.2 mg, 1.96 mmol, 1.8 eq) in DCM (8 mL) was added azido(trimethyl)silane (627.3 mg, 5.45 mmol, 5 eq) dropwise at room temperature under N₂ atmosphere. The mixture was stirred at room temperature for 20 min. Then 2-chloro-7-methyl-6,7-dihydro-5H-cyclopenta[b]pyridin-7-ol (200 mg, 1.09 mmol, 1 eq) was added. The reaction mixture was stirred for 1 h at room temperature under the N₂ atmosphere. The resulting mixture was concentrated under reduced pressure. The residue was purified by silica gel column chromatography, eluted with PE/EtOAc (4:1) to afford 7-azido-2-chloro-7-methyl-6,7-dihydro-5H-cyclopenta[b]pyridine (103 mg, 493.66 umol, 45%) as a yellow oil. LCMS (ES, m/z): 209, 211 [M+H]⁺, Rt 0.935 min.

### Step 3. 2-chloro-7-methyl-6,7-dihydro-5H-cyclopenta[b]pyridin-7-amine

The mixture of 7-azido-2-chloro-7-methyl-6,7-dihydro-5H-cyclopenta[b]pyridine (800 mg, 383.42 umol, 1 eq), Zn (125.36 mg, 1.92 mmol, 5 eq) and NH₄Cl (205.1 mg, 3.83 mmol, 10 eq) in MeOH (15 mL) and H₂O (5 mL) was stirred for 1 h at 75° C. under N₂ atmosphere. The mixture was allowed to cool down to room temperature. The resulting mixture was filtered, the filter cake was washed with DCM (3×20 mL). The filtrate was concentrated under reduced pressure to afford 2-chloro-7-methyl-6,7-dihydro-5H-cyclopenta[b]pyridin-7-amine (500 mg, crude) as a yellow solid. ¹H NMR (300 MHz, DMSO-d6) δ 7.84 (d, J=7.8 Hz, 1H), 7.45 (d, J=7.8 Hz, 1H), 2.99-2.81 (m, 2H), 2.21 (t, J=6.9 Hz, 2H), 1.63 (s, 3H). LCMS (ES, m/z): 183, 185 [M+H]⁺, Rt 0.445 min.

### Step 4. tert-butyl (2-chloro-7-methyl-6,7-dihydro-5H-cyclopenta[b]pyridin-7-yl)carbamate

To a stirred solution of 2-chloro-7-methyl-5,6-dihydrocyclopenta[b]pyridin-7-amine (100 mg, 547.50 umol, 1 eq) and TEA (166.2 mg, 1.64 mmol, 3 eq) in DCM (4 mL) was added Boc₂O (238.98 mg, 1.09 mmol, 2 eq) portionwise at room temperature. The resulting mixture was stirred for 2 h at 25° C. The mixture was concentrated under reduced pressure. The residue was purified by silica gel column chromatography, eluted with PE/EtOAc (1:5) to afford tert-butyl N-(2-chloro-7-methyl-5,6-dihydrocyclopenta[b]pyridin-7-yl)carbamate (57 mg, 181.42 umol, 33% yield) as a yellow oil. LCMS (ES, m/z): 283, 285 [M+H]⁺, Rt 1.067 min.

### Step 5. tert-butyl (S)-(2-chloro-7-methyl-6,7-dihydro-5H-cyclopenta[b]pyridin-7-yl)carbamate and tert-butyl (R)-(2-chloro-7-methyl-6,7-dihydro-5H-cyclopenta[b]pyridin-7-yl)carbamate

Tert-butyl N-(2-chloro-7-methyl-5,6-dihydrocyclopenta[b]pyridin-7-yl)carbamate (557 mg, 1.97 mmol, 1 eq) was separated by Chiral-HPLC: (Column: CHIRALPAK IG, 3*25 cm/5 μm; Mobile Phase A: CO₂, Mobile Phase B: MeOH (0.1% 2M NH₃-MeOH); Flow rate: 80 mL/min; Gradient: 10% B; Column Temp: 35° C.; Back Pressure: 100 bar; 220 nm; RT1: 4.31 min; RT2: 8.13 min) to afford tert-butyl (S)-(2-chloro-7-methyl-6,7-dihydro-5H-cyclopenta[b]pyridin-7-yl)carbamate (250 mg, 848.76 umol, 45% yield) as a yellow oil and tert-butyl (R)-(2-chloro-7-methyl-6,7-dihydro-5H-cyclopenta[b]pyridin-7-yl)carbamate (256 mg, 869.13 umol, 46% yield) as a yellow oil. LCMS (ES, m/z): 283, 285 [M+H]⁺, Rt 1.067 min.

Absolute stereochemistry of title compounds were assigned arbitrarily upon chiral separation.

### Intermediate 184. 3-bromo-1-ethyl-1H-pyrazole

To a stirred solution of 3-bromo-1H-pyrazole (1 g, 5.16 mmol, 1 eq.) in DMF (10 mL) was added NaH (412.39 mg, 10.31 mmol, 60% purity, 2 eq.) in portions. The reaction mixture was stirred at 0° C. for 0.5 h before iodoethane (1.61 g, 10.31 mmol, 2 eq.) was added dropwise. The reaction mixture was stirred for 12 h at room temperature and monitored by LCMS. The reaction was quenched with NH₄Cl (100 mL) and extracted with EA (3×80 mL). The combined organic layers were washed with brine (2×150 mL), dried over anhydrous Na₂SO₄. After filtration, the filtrate was concentrated under reduced pressure. The residue was purified by silica gel column chromatography (eluting with 1:1 EA/PE) to afford 1-ethyl-3-iodo-pyrazole (600 mg, 3.43 mmol, 66% yield) as a yellow liquid. ¹HNMR (400 MHz, DMSO-d₆) δ 7.77 (d, J=2.4 Hz, 1H), 6.35 (d, J=2.4 Hz, 1H), 4.17-4.06 (m, 2H), 1.35 (t, J=7.2 Hz, 3H). LCMS (ES, m/z): 175, 177 [M+H]⁺, Rt 0.560 min.

### Intermediate 185. 3-bromo-1-cyclopropyl-1H-pyrazole

To a stirred mixture of 3-bromo-1H-pyrazole (500 mg, 3.40 mmol, 1 eq.) and cyclopropylboronic acid (876.66 mg, 10.21 mmol, 3 eq.) in DCE (20 mL) were added Cu(OAc)₂ (679.20 mg, 3.40 mmol, 1 eq.), 2,2-bipyridyl (1.06 g, 6.80 mmol, 2 eq.) and Na₂CO₃ (1.08 g, 10.21 mmol, 3 eq.) in portions at room temperature. The reaction mixture was stirred for 16 h at 65° C. under O₂ atmosphere and was allowed to cool down to room temperature. The resulting mixture was filtered, the filter cake was washed with DCM (3×10 mL) and filtrate was concentrated under reduced pressure. The residue was purified by silica gel column chromatography, eluted with EA/PE (1:1) to afford 3-bromo-1-cyclopropyl-1H-pyrazole (350 mg, 1.87 mmol, 55% yield) as a light-yellow oil. ¹H NMR (300 MHz, DMSO-d₆) δ 7.73-7.65 (m, 1H), 6.23 (d, J=2.4 Hz, 1H), 3.65-3.52 (m, 1H), 0.97-0.76 (m, 4H). LCMS (ES, m/z):187, 189 [M+H]⁺, Rt 0.750 min.

### Intermediate 186. 3-bromo-1-isopropyl-pyrazole

Following the procedure for Intermediate 184, 3-bromo-1-isopropyl-pyrazole (720 mg, 3.43 mmol, 50.38% yield) was obtained as a light-yellow oil, replacing EtI with 2-iodopropane. ¹H NMR (300 MHz, DMSO-d₆) δ 7.67 (d, J=2.4 Hz, 1H), 6.22 (d, J=2.4 Hz, 1H), 4.45-4.26 (m, 1H), 1.26 (d, J=6.6 Hz, 6H). LCMS (ES, m/z):189, 191 [M+H]⁺, Rt 0.625 min.

### Intermediate 187. 3-bromo-1-(oxetan-3-yl)pyrazole

To a stirred solution of 3-bromo-1H-pyrazole (2 g, 13.608 mmol, 1 eq) in DMF (30 mL) was added NaHMDS (4.99 g, 27.216 mmol, 2 eq.) dropwise at 0° C. under N₂ atmosphere. The resulting mixture was stirred for 36 h at 45° C. under N₂ atmosphere. The reaction was quenched by the addition of saturated NH₄Cl solution (200 mL) at 0° C. and mixture was extracted with EA (3×100 mL). The combined organic layers were washed with brine (2×150 mL), dried over anhydrous Na₂SO₄. After filtration, the filtrate was concentrated under reduced pressure. The residue was purified by silica gel column chromatography, eluted with [PE:EA](1:2) to afford 3-bromo-1-(oxetan-3-yl)pyrazole (1 g, 4.93 mmol, 36%) as a yellow oil. LCMS (ES, m/z): 203, 205 [M+H]⁺, Rt 0.556 min.

### Intermediate 188. 3-iodo-1-(trifluoromethyl)pyrazole

### Step 1. 1-[bromo(difluoro)methyl]-3-iodo-pyrazole

Following the procedure for Intermediate 184, 1-[bromo(difluoro)methyl]-3-iodo-pyrazole (1.8 g, crude) was obtained as a yellow oil, starting from 3-iodo-1H-pyrazole (2 g, 10.31 mmol, 1 eq.) and dibromo(difluoro)methane (3.25 g, 15.47 mmol, 1.5 eq.). 1H NMR (300 MHz, DMSO-d6) δ 8.33 (d, J=2.7 Hz, 1H), 6.87 (d, J=2.7 Hz, 1H). LCMS (ES, m/z): 323, 325 [M+H]⁺, Rt 0.905 min.

### Step 2. 3-iodo-1-(trifluoromethyl)pyrazole

To a stirred solution of 1-[bromo(difluoro)methyl]-3-iodo-pyrazole (1.3 g, 4.03 mmol, 1 eq.) in DCM (20 mL) was added silver tetrafluoroborate (2.35 g, 12.08 mmol, 3 eq.) in portions at −78° C. under N₂ atmosphere. The resulting mixture was stirred at room temperature for 16 h under the N₂ atmosphere. The reaction was quenched with ice/water (100 mL) and the mixture was extracted with DCM (3×50 mL). The combined organic layer was washed with brine (100 mL). The organic layer was dried over anhydrous Na₂SO₄. After filtration, the filtrate was concentrated to 5-10 mL to afford 3-iodo-1-(trifluoromethyl)pyrazole (crude) as a DCM solution. LCMS (ES, m/z): 263 [M+H]⁺, Rt 0.861 min.

### Intermediate 189. 1-(difluoromethyl)-3-iodo-1H-pyrazole

Following the procedure for Intermediate 184, 1-(difluoromethyl)-3-iodo-1H-pyrazole (0.9 g, 3.69 mmol, 72% yield) was obtained as a colorless oil, starting from 3-iodo-1H-pyrazole (1 g, 5.16 mmol, 1 eq) and difluoro(iodo)methane (9.17 g, 51.55 mmol, 10 eq). ¹H NMR (400 MHz, DMSO-d6) δ 8.17 (d, J=2.8 Hz, 1H), 7.95-7.66 (m, 1H), 6.77 (d, J=2.8 Hz, 1H). ¹⁹F NMR (400 MHz, DMSO-d6) δ −94.43. LCMS (ES, m/z): 245 [M+H]⁺, Rt 0.781 min.

### Intermediate 190. tert-butyl (1-(6-bromopyridin-2-yl)cyclopropyl)(methyl)carbamate

### Step 1. 1-(6-bromopyridin-2-yl)cyclopropan-1-amine

To a solution of 6-bromopyridine-2-carbonitrile (1 g, 5.46 mmol, 1 eq) in diethyl ether (10 mL) were added Ti(OiPr)₄ (1.55 g, 5.46 mmol, 1 eq) and bromo(ethyl)magnesium (1 M in THF, 6.56 mL, 1.2 eq) dropwise at −78° C. under N₂ atmosphere. The mixture was stirred at −78° C. for 1 h. Then the mixture was warmed to room temperature, stirred for an additional 4 h and quenched with ice/water (150 mL). The mixture was extracted with EA (3×100 mL). The combined organic layer was washed with brine (200 mL), dried over sodium sulfate, filtered and concentrated. The residue was purified by silica gel column, eluted with EA:PE (1:3) to afford 1-(6-bromopyridin-2-yl)cyclopropan-1-amine (230 mg, 1.08 mmol, 20% yield) as a yellow solid. ¹H NMR (300 MHz, DMSO-d6) δ 7.79-7.76 (m, 1H), 7.67 (t, J=6.0 Hz, 1H), 7.36-7.34 (m, 1H), 1.18-1.16 (m, 2H), 1.02-0.99 (d, J=6.6 Hz, 2H). LCMS (ES, m/z): 213, 215 [M+H]⁺, Rt 0.434 min.

### Step 2. tert-butyl (1-(6-bromopyridin-2-yl)cyclopropyl)carbamate

To a solution of 1-(6-bromo-2-pyridyl)cyclopropanamine (800 mg, 3.75 mmol, 1 eq) and TEA (1.14 g, 11.26 mmol, 1.57 mL, 3 eq) in DCM (25 mL) was added Boc₂O (983.32 mg, 4.51 mmol, 2 eq) portionwise. The mixture was stirred at room temperature for 16 h. Then ice/water (100 mL) was added and mixture was extracted with EA (3×80 mL). The combined organic layer was washed with brine (150 mL), dried over sodium sulfate, filtered and concentrated. The residue was purified by silica gel column, eluted with EA:PE (1:4) to afford tert-butyl (1-(6-bromopyridin-2-yl)cyclopropyl)carbamate (1.05 g, 3.35 mmol, 89.29% yield) as yellow solid. LCMS (ES, m/z): 313, 315 [M+H]⁺, Rt 1.112 min.

### Step 3. tert-butyl (1-(6-bromopyridin-2-yl)cyclopropyl)(methyl)carbamate

To a solution of tert-butyl (1-(6-bromopyridin-2-yl)cyclopropyl)carbamate (1 g, 3.19 mmol, 1 eq) in DMF (20 mL) was added NaH (383.12 mg, 9.58 mmol, 60% purity, 1.5 eq) in portions at 0° C. under N₂ atmosphere. The mixture was stirred at 25° C. for 1 h before Mel (679.80 mg, 4.79 mmol, 2 eq) was added dropwise at 0° C. under the N₂ atmosphere. The mixture was stirred at 25° C. for 4 h. Then ice/water (100 mL) was added and the mixture was extracted with EA (3×80 mL). The combined organic layer was washed with brine (2×150 mL), dried over sodium sulfate, filtered and concentrated. The residue was purified by silica gel column, eluted with EA:PE (1:3) to afford tert-butyl (1-(6-bromopyridin-2-yl)cyclopropyl)(methyl)carbamate (960 mg, 2.93 mmol, 92% yield) as yellow solid. LCMS (ES, m/z): 327, 329 [M+H]⁺, Rt 0.809 min.

### Intermediate 191. tert-butyl 3-(6-bromopyridin-2-yl)-3-fluoroazetidine-1-carboxylate

### Step 1. tert-butyl 3-(6-bromopyridin-2-yl)-3-hydroxyazetidine-1-carboxylate

To a solution of 2,6-dibromopyridine (4 g, 17.02 mmol, 1 eq.) in THF (50 mL) was added n-BuLi (2.5M in hexane, 17.02 mL, 42.55 mmol, 2.5 eq.) at −78° C. under N₂ atmosphere. The resulting mixture was stirred for 30 min at −78° C. Then tert-butyl 3-oxoazetidine-1-carboxylate (2.91 g, 17.02 mmol, 1 eq.) dissolved in THF (5 mL) was added to the above solution at −78° C. The resulting mixture was stirred for 1.5 h at −78° C. The reaction was quenched with aq. saturated NH₄Cl (200 mL) at 0° C. The resulting mixture was extracted with EA (3×150 mL). The combined organic layers were washed with brine (2×300 mL), dried over anhydrous Na₂SO₄. After filtration, the filtrate was concentrated under reduced pressure. The residue was purified by silica gel column chromatography, eluted with PE:EA (1:1) to afford tert-butyl 3-(6-bromopyridin-2-yl)-3-hydroxyazetidine-1-carboxylate (2.1 g, 6.38 mmol, 38%) as a yellow oil. LCMS (ES, m/z): 273, 275 [M+H-56]⁺, Rt 0.998 min.

### Step 2. tert-butyl 3-(6-bromopyridin-2-yl)-3-fluoroazetidine-1-carboxylate

To a solution of tert-butyl 3-(6-bromopyridin-2-yl)-3-hydroxyazetidine-1-carboxylate (2.1 g, 6.40 mmol, 1 eq.) in DCM (30 mL) was added DAST (2.06 g, 12.80 mmol, 2.5 eq.) at 0° C. The resulting mixture was stirred for 3 h at 25° C. The reaction was quenched with ice/water (150 mL) at 0° C. and was extracted with EA (3×100 mL). The combined organic layers were washed with brine (2×200 mL), dried over anhydrous Na₂SO₄. After filtration, the filtrate was concentrated under reduced pressure. The residue was purified by silica gel column chromatography, eluted with PE:EA (1:1) to afford tert-butyl 3-(6-bromopyridin-2-yl)-3-fluoroazetidine-1-carboxylate (1.3 g, 3.92 mmol, 62%) as a yellow oil. ¹H NMR (400 MHz, DMSO-d6) δ 7.87 (t, J=7.6 Hz, 1H), 7.74 (d, J=8.0 Hz, 1H), 7.63-7.60 (m, 1H), 4.40-4.22 (m, 4H), 1.43 (s, 9H). LCMS (ES, m/z): 275, 277 [M+H-56]⁺, Rt 1.148 min.

### Intermediate 192. tert-butyl (2-(6-bromopyridin-2-yl)propan-2-yl)carbamate

### Step 1. N-[1-(6-bromo-2-pyridyl)-1-methyl-ethyl]acetamide

To a solution of 2-(6-bromo-2-pyridyl)propan-2-ol (2 g, 9.26 mmol, 1 eq.) in acetonitrile (30 mL) was added boron trifluoride etherate (3.28 g, 23.14 mmol, 2.5 eq.) under N₂ at room temperature. The resulting mixture was stirred for 16 h at 80° C. and was allowed to cool down to room temperature. The mixture was neutralized with NaOH (5M) and extracted with DCM (3×100 mL). The organic phase was washed with brine (250 mL) and dried over Na₂SO₄. The filtrate was concentrated under reduced pressure. The residue was purified by silica gel column, eluted with EA/PE (1:1) to afford N-[1-(6-bromo-2-pyridyl)-1-methyl-ethyl]acetamide (920 mg, 3.53 mmol, 38% yield) as a white oil. LCMS (ES, m/z): 257, 259 [M+H]⁺, Rt 0.798 min.

### Step 2. 2-(6-bromo-2-pyridyl)propan-2-amine

A solution of N-[1-(6-bromo-2-pyridyl)-1-methyl-ethyl]acetamide (920 mg, 3.58 mmol, 1 eq.) in HCl (20 mL) was stirred for 16 h at 100° C. The reaction was allowed to cool down to room temperature. The mixture was neutralized with NaOH (2M) to pH=7-8 and extracted with DCM (3×100 mL). The organic phase was washed with brine (150 mL) and dried over Na₂SO₄. The filtrate was concentrated under reduced pressure. The residue was purified by Prep-TLC, eluted with MeOH/DCM (1:10) to afford 2-(6-bromo-2-pyridyl)propan-2-amine (430 mg, 56% yield) as a yellow oil. ¹H NMR (300 MHz, DMSO-d6) δ 7.71-7.67 (m, 2H), 7.45 (d, J=6.0 Hz, 1H), 1.35 (s, 6H). LCMS (ES, m/z): 215, 217 [M+H]⁺, Rt 0.798 min.

### Step 3. tert-butyl (2-(6-bromopyridin-2-yl)propan-2-yl)carbamate

To a stirred solution of 2-(6-bromo-2-pyridyl)propan-2-amine (300 mg, 1.39 mmol, 1 eq.) in DCM (10 mL) was added TEA (423.41 mg, 4.18 mmol, 3 eq.) and Boc₂O (365.29 mg, 1.67 mmol, 1.2 eq.) in DCM (2 mL) at 0° C. The resulting mixture was stirred for 1 h at room temperature. Desired product was detected by LCMS. The resulting mixture was concentrated under reduced pressure. The residue was purified by silica gel column chromatography, eluted with EA/PE (1:3) to afford tert-butyl (2-(6-bromopyridin-2-yl)propan-2-yl)carbamate (345 mg, 1.10 mmol, 79% yield) as a yellow oil. LCMS (ES, m/z): 315, 317 [M+H]⁺, Rt 1.111 min.

### Intermediate 193. 6-bromo-N,N-dimethyl-pyridine-2-carboxamide

To a stirred mixture of 6-bromopyridine-2-carboxylic acid (2 g, 9.90 mmol) in DMF (20 mL) was added N-methylmethanamine;hydrochloride (888.09 mg, 10.89 mmol), N,N,N′,N′-tetramethyl-1-(3-oxido-2,3-dihydrotriazolo[4,5-b]pyridin-3-ium-1-yl)methanediamine;hexafluorophosphate (5.65 g, 14.85 mmol) and N-ethyl-N-isopropyl-propan-2-amine (2.56 g, 19.80 mmol, 3.45 mL) at 0° C. The resulting mixture was stirred for 2 h at r.t. The reaction was monitored by LC-MS. The resulting mixture was washed with brine (60 mL). The resulting mixture was extracted with EA (3×60 mL). The combined organic layers were dried over anhydrous Na₂SO₄. The resulting mixture was concentrated under reduced pressure. The residue was purified by silica gel column chromatography (eluting with 1:1 EA/PE) to afford 6-bromo-N,N-dimethyl-pyridine-2-carboxamide (2 g, 8.73 mmol, 88.18% yield, 100% purity) as a white solid. LCMS (ES, m/z): 230 [M+H]⁺, Rt 0.571 min.

### Intermediate 195. tert-butyl (R)-(1-(6-bromopyridin-2-yl)-2,2,2-trifluoroethyl)carbamate

### Step 1. (S)—N-((6-bromopyridin-2-yl)methylene)-2-methylpropane-2-sulfinamide

To a stirred solution of 6-bromopyridine-2-carbaldehyde (2 g, 10.75 mmol, 1 eq.) in DCM (30 mL) was added (S)-2-methylpropane-2-sulfinamide (1.30 g, 10.75 mmol, 1 eq.) in portions. The reaction mixture was stirred at room temperature for 18 h and then concentrated under vacuum to afford (S)—N-((6-bromopyridin-2-yl)methylene)-2-methylpropane-2-sulfinamide (3.0 g, crude) as a white solid. LCMS (ES, m/z): 289, 291 [M+H]⁺, Rt 0.859 min.

### Step 2. (S)—N—((R)-1-(6-bromopyridin-2-yl)-2,2,2-trifluoroethyl)-2-methylpropane-2-sulfinamide

To a solution of (S)—N-((6-bromopyridin-2-yl)methylene)-2-methylpropane-2-sulfinamide (1 g, 3.46 mmol, 1 eq.) in THF (15 mL) was added tetramethylammonium fluoride (0.97 g, 10.37 mmol, 3 eq.) at 0° C. under the N₂ atmosphere. The reaction mixture was stirred at −60° C. for 10 mins. Then a solution of trimethyl(trifluoromethyl)silane (737.55 mg, 5.19 mmol, 1.5 eq.) in THF (0.5 mL) was added dropwise and the mixture was stirred for another 1 h at −60° C. The reaction was quenched by the addition of ice/water (100 mL). The resulting mixture was extracted with EA (3×80 mL). The combined organic layers were washed with brine (150 mL), dried over anhydrous Na₂SO₄. After filtration, the filtrate was concentrated under reduced pressure. The residue was purified by silica gel column chromatography, eluted with EA/PE (2:1) to afford (S)—N—((R)-1-(6-bromopyridin-2-yl)-2,2,2-trifluoroethyl)-2-methylpropane-2-sulfinamide (650 mg, 1.81 mmol, 52% yield) as a yellow solid. LCMS (ES, m/z): 359, 361 [M+H]⁺, Rt 0.919 min.

### Step 3. (R)-1-(6-bromopyridin-2-yl)-2,2,2-trifluoroethan-1-amine

To a stirred solution of (S)—N—((R)-1-(6-bromopyridin-2-yl)-2,2,2-trifluoroethyl)-2-methylpropane-2-sulfinamide (500 mg, 1.39 mmol, 1 eq.) in DCM (8 mL) were added HCl (4 M in dioxane, 1 mL). The mixture was stirred for 2 h at room temperature. The resulting mixture was concentrated under vacuum to afford (R)-1-(6-bromopyridin-2-yl)-2,2,2-trifluoroethan-1-amine (510 mg, crude) as a white solid. LCMS (ES, m/z): 255, 257 [M+H]⁺, Rt 0.466 min.

### Step 4. tert-butyl (R)-(1-(6-bromopyridin-2-yl)-2,2,2-trifluoroethyl)carbamate

To a solution of (R)-1-(6-bromopyridin-2-yl)-2,2,2-trifluoroethan-1-amine (510 mg, 2.00 mmol, 1 eq.), pyridine (474.54 mg, 6.00 mmol, 3 eq.) and DMAP (48.86 mg, 399.95 μmol. 0.2 eq.) in 1,4-dioxane (10 mL) was added Boc₂O (654.66 mg, 3.00 mmol, 1.5 eq.) at 0° C. The mixture was stirred for 16 h at room temperature. The resulting mixture was concentrated under reduced pressure. The residue was purified by silica gel column chromatography, eluted with EA/PE (1:1) to afford tert-butyl (R)-(1-(6-bromopyridin-2-yl)-2,2,2-trifluoroethyl)carbamate (345 mg, 951.99 μmol, 48% yield) as a white solid. LCMS (ES, m/z): 355, 357 [M+H]⁺, Rt 0.858 min.

### Intermediate 200. (6-bromopyridin-2-yl)(methyl)(methylimino)-l6-sulfanone

### Step 1. N-((6-bromopyridin-2-yl)(methyl)-λ4-sulfaneylidene)-2,2,2-trifluoroacetamide

To a stirred solution of NaH (60% in oil, 528 mg, 13.20 mmol, 8 eq.) in THF (15 mL) was added 2-bromo-6-methylsulfanyl-pyridine (3 g, 14.70 mmol, 1 eq.) and 2,2,2-trifluoroacetamide (2.49 g, 22.05 mmol, 1.5 eq.) in THF (30 mL) dropwise at 0° C. under N₂ atmosphere. After 5 min, a solution of 1,3-dibromo-5,5-dimethyl-imidazolidine-2,4-dione (6.30 g, 22.05 mmol, 1.5 eq.) in THF (20 mL) was added dropwise. The resulting mixture was stirred for 1 h at 0° C. under the N₂ atmosphere. The reaction was quenched with aq. Na₂SO₃ (25%, 150 mL) and mixture was extracted with EA (3×250 mL). The combined organic layers were washed with brine (500 mL), dried over anhydrous Na₂SO₄. After filtration, the filtrate was concentrated under reduced pressure to afford N-((6-bromopyridin-2-yl)(methyl)-λ4-sulfaneylidene)-2,2,2-trifluoroacetamide (4.3 g, crude) as a white solid. LCMS (ES, m/z): 315, 317 [M+H]⁺. Rt 0.653 min.

### Step 2. (6-bromopyridin-2-yl)(imino)(methyl)-l6-sulfanone

To a stirred solution of N-((6-bromopyridin-2-yl)(methyl)-λ4-sulfaneylidene)-2,2,2-trifluoroacetamide (4.3 g, 13.65 mmol, 1 eq.) in MeOH (40 mL), water (40 mL) and sulfolane (7 mL) was added Oxone (14 g, 40.43 mmol, 3 eq.) in portions. The mixture was adjusted to pH=10 with K₂CO₃ (10.8 g, 78.14 mmol, 6 eq.). The resulting mixture was stirred at room temperature for 16 h under the N₂ atmosphere. Then MeOH was removed under reduced pressure. The residue was partitioned between DCM (100 mL) and Na₂SO₃ (10%, 100 mL). The organic phase was washed with water (2×100 mL), dried over anhydrous MgSO₄. After filtration, the filtrate was concentrated to afford (6-bromopyridin-2-yl)(imino)(methyl)-l6-sulfanone (1.2 g, crude) as an off-white solid. LCMS (ES, m/z): 235, 237 [M+H]⁺. Rt 0.450 min.

### Step 3. (6-bromopyridin-2-yl)(methyl)(methylimino)-l6-sulfanone

To a stirred solution of (6-bromopyridin-2-yl)(imino)(methyl)-l6-sulfanone (1.2 g, 5.10 mmol, 1 eq.) in formic acid (48 mL) was added formaldehyde (1.53 g, 51.04 mmol, 10 eq) in portions at room temperature. The resulting mixture was stirred for 4 h at 100° C. The mixture was allowed to cool down to room temperature and slowly quenched with saturated NaHCO₃ (300 mL) and extracted with DCM (3×300 mL). The organic phase was dried over anhydrous MgSO₄. After filteration, the filtrate was concentrated under vacuum. The residue was purified by silica gel column chromatography (eluting with 1:10 MeOH/DCM) to afford (6-bromopyridin-2-yl)(methyl)(methylimino)-l6-sulfanone (675 mg, 2.63 mmol, 51% yield) as a yellow oil. LCMS (ES, m/z): 249, 251 [M+H]⁺. Rt 0.167 min.

### Intermediate 200A and Intermediate 200B. (R)-(6-bromopyridin-2-yl)(methyl)(methylimino)-l6-sulfanone and (S)-(6-bromopyridin-2-yl)(methyl)(methylimino)-l6-sulfanone

Racemic (6-bromopyridin-2-yl)(methyl)(methylimino)-l6-sulfanone (Intermediate 200, 910 mg, 3.64 mmol, 1 eq.) was separated by Chiral-SFC (Column: CHIRAL ART Cellulose-SC, 7*25 cm/10 μm; Mobile Phase A: CO₂, Mobile Phase B: IPA (0.5% 2M NH₃-MeOH); Flow rate: 200 mL/min; Gradient: isocratic 40% B; Column Temperature: 35° C.; Back Pressure: 100 bar; 220 nm; RT1(min): 21.18; RT2(min): 24.01) to afford (R)-(6-bromopyridin-2-yl)(methyl)(methylimino)-l6-sulfanone (235 mg, 0.94 mmol, 26% yield) and (S)-(6-bromopyridin-2-yl)(methyl)(methylimino)-l6-sulfanone (217 mg, 0.87 mmol, 24% yield) as a white solid. LCMS (ES, m/z): 249, 251 [M+H]⁺. Rt 0.167 min.

Absolute stereochemistry of title compounds were assigned arbitrarily upon chiral separation.

### Intermediate 202. 6-chloro-2-isopropylpyridazin-3-one

To a stirred solution of 3-chloro-1H-pyridazin-6-one (1 g, 7.66 mmol, 1 eq.) in DMF (10 mL) was added 2-iodopropane (1.30 g, 7.66 mmol, 1 eq.) and Cs₂CO₃ (7.49 g, 22.98 mmol, 3 eq.) mixture was then stirred at 25° C. for 1 h. LCMS indicated that about 45% of the target product was generated. The reaction was quenched with H₂O (100 mL). The resulting mixture was extracted with EA (3×80 mL). The combined organic layers were washed with brine (2×150 mL), dried over anhydrous Na₂SO₄. After filtration, the filtrate was concentrated under reduced pressure. The residue was purified by silica gel column chromatography, eluted with [EA/PE](1:1) to afford 6-chloro-2-isopropylpyridazin-3-one (320 mg, 1.86 mmol, 24% yield) as a white solid. ¹HNMR (300 MHz, DMSO-d6) δ 7.52 (d, J=9.6 Hz, 1H), 7.01 (d, J=9.6 Hz, 1H), 5.14-4.99 (m, 1H), 1.26 (d, J=6.6 Hz, 6H). LCMS (ES, m/z): 173 [M+H]⁺, Rt 0.588 min.

### Intermediate 205 and 206. tert-butyl (S)-3-(6-bromopyridin-2-yl)morpholine-4-carboxylate and tert-butyl (R)-3-(6-bromopyridin-2-yl)morpholine-4-carboxylate

### Step 1. tert-butyl 3-(6-bromo-2-pyridyl)morpholine-4-carboxylate

A mixture of 2,6-dibromopyridine (300 mg, 1.27 mmol, 1 eq.), 4-tert-butoxycarbonylmorpholine-3-carboxylic acid (439.27 mg, 1.90 mmol, 1.5 eq.), [Ir(dF(Me)ppy)2(dtbbpy)]PF₆ (12.94 mg, 12.66 umol, 0.01 eq.), dtbbpy (33.94 mg, 126.64 umol, 0.1 eq.), NiCl₂(DME) (39.13 mg, 193.72 umol, 0.15 eq.) and Cs₂CO₃ (825.23 mg, 2.53 mmol, 2 eq.) in DMF (10 mL) was irradiated with blue light (450 nm) for 2 h at 50° C. under nitrogen atmosphere. The reaction was monitored by TLC. The resulting mixture was cooled down to room temperature, and diluted with brine (80 mL). The mixture was extracted with EA (3×50 mL). The combined organic layers were dried over anhydrous Na₂SO₄. After filtration, the filtrate was concentrated under reduced pressure. The residue was purified by silica gel column chromatography (eluting with 1:4 EA/PE) to afford tert-butyl 3-(6-bromo-2-pyridyl)morpholine-4-carboxylate (120 mg, 349.85 umol, 28% yield) as a white solid. LCMS (ES, m/z): 343, 345 [M+H]⁺, Rt 0.760 min.

### Step 2. tert-butyl (S)-3-(6-bromopyridin-2-yl)morpholine-4-carboxylate and tert-butyl (R)-3-(6-bromopyridin-2-yl)morpholine-4-carboxylate

The racemic tert-butyl 2-(6-bromo-2-pyridyl)morpholine-4-carboxylate (460 mg, 1.34 mmol, 1 eq.) was separated by Chiral-HPLC (Column: CHIRALPAK AD-H, 2*25 cm/5 μm; Mobile Phase A: Hex0.1% DEA, Mobile Phase B: IPA; Flow rate: 20 mL/min; Gradient: 7% B in 10 min; Wave Length: 220/254 nm; RT1(min): 8.63; RT2(min): 9.92) to afford tert-butyl (S)-3-(6-bromopyridin-2-yl)morpholine-4-carboxylate (180 mg, 524.78 umol, 39% yield) as a white solid and tert-butyl (R)-3-(6-bromopyridin-2-yl)morpholine-4-carboxylate (180 mg, 524.78 umol, 39% yield) as a white solid. LCMS (ES, m/z): 343, 345 [M+H]⁺, Rt 0.778 min.

Absolute stereochemistry of title compounds were assigned arbitrarily upon chiral SFC separation.

### Intermediate 207 and 208. tert-butyl (S)-2-(6-bromopyridin-2-yl)morpholine-4-carboxylate and tert-butyl (R)-2-(6-bromopyridin-2-yl)morpholine-4-carboxylate

### Step 1. tert-butyl 2-(6-bromo-2-pyridyl)morpholine-4-carboxylate

A solution of 2,6-dibromopyridine (100 mg, 422.13 umol, 1 eq.), 4-tert-butoxycarbonylmorpholine-2-carboxylic acid (117.14 mg, 506.56 umol, 1.2 eq.), [Ir(dF(Me)ppy)2(dtbbpy)]PF₆ (4.28 mg, 4.22 umol, 0.01 eq.), NiBr₂(DME) (13.00 mg, 42.21 umol, 0.1 eq.), Cs₂CO₃ (275.08 mg, 844.27 umol, 2 eq.) and 5,5′-dimethyl-2,2′-dipyridyl (14.44 mg, 63.32 umol, 0.15 eq.) in DMF (12 mL) was irradiated with blue light for 6 h at 50° C. under nitrogen atmosphere. The reaction was monitored by LC-MS. The mixture was allowed to cool down to room temperature. The resulting mixture was diluted with brine (60 mL), and extracted with EA (3×40 mL). The combined organic layers were dried over anhydrous Na₂SO₄. After filtration, the filtrate was concentrated under reduced pressure. The residue was purified by silica gel column chromatography (eluting with 1:3 EA/PE) to afford tert-butyl 2-(6-bromo-2-pyridyl)morpholine-4-carboxylate (45 mg, 124.56 umol, 29.51% yield) as a colorless oil. LCMS (ES, m/z): 343, 345 [M+H]⁺, Rt 0.778 min.

### Step 2. tert-butyl (S)-2-(6-bromopyridin-2-yl)morpholine-4-carboxylate and tert-butyl (R)-2-(6-bromopyridin-2-yl)morpholine-4-carboxylate

The racemic tert-butyl 2-(6-bromo-2-pyridyl)morpholine-4-carboxylate (420 mg, 1.22 mmol) was separated by Chiral-HPLC (Column: (R, R)-WHELK-O1-Kromasil/2.11*25 cm/5 μm; Mobile Phase A: Hex (0.5% 2M NH₃-MeOH), Mobile Phase B: EtOH; Flow rate: 20 mL/min; Gradient: 5% B in 29 min; Wave Length: 220/254 nm; RT1(min): 22.89; RT2(min): 25.67) to afford tert-butyl (S)-2-(6-bromopyridin-2-yl)morpholine-4-carboxylate (160 mg, 466.47 umol, 38% yield) as a colorless oil and tert-butyl (R)-2-(6-bromopyridin-2-yl)morpholine-4-carboxylate (170 mg, 495.63 umol, 40% yield) as a colorless oil. LCMS (ES, m/z): 343, 345 [M+H]⁺, Rt 0.778 min.

Absolute stereochemistry of title compounds were assigned arbitrarily upon chiral separation.

### Intermediate 210. tert-butyl N-[(2-bromo-4-pyridyl)methyl]-N-methyl-carbamate

Following the 2-step procedure described for Intermediate 2, tert-butyl N-[(2-bromo-4-pyridyl)methyl]-N-methyl-carbamate (1.38 g, 84% yield) was obtained as a yellow oil, starting from 2-bromopyridine-4-carbaldehyde (1 g, 5.38 mmol, 1 eq.).

¹H NMR (400 MHz, DMSO-d₆) δ 8.35 (d, J=5.2 Hz, 1H), 7.44 (s, 1H), 7.25 (d, J=4.4 Hz, 1H), 4.40 (s, 2H), 2.84 (s, 3H), 2.49-2.28 (m, 9H). LCMS (ES, m/z): 301, 303 [M+H]⁺, Rt 1.633 min.

### Intermediate 211 and Intermediate 212. tert-butyl (R)-(1-(2-bromo-6-methylpyridin-4-yl)ethyl)(methyl)carbamate and tert-butyl (S)-(1-(2-bromo-6-methylpyridin-4-yl)ethyl)(methyl)carbamate

### Step 1. 1-(2-bromo-6-methyl-4-pyridyl)ethanone oxime

To a solution of 1-(2-bromo-6-methyl-4-pyridyl)ethanone (3.7 g, 17.28 mmol, 1 eq.) in pyridine (20 mL) was added hydroxylamine hydrochloride (2.40 g, 34.57 mmol, 2 eq.) at room temperature and stirred for 12 h. The reaction mixture was concentrated, washed with DCM (3×30 mL) to afford 1-(2-bromo-6-methyl-4-pyridyl)ethanone oxime (3.2 g, crude) as a yellow solid. LCMS (ES, m/z): 229, 231 [M+H]⁺, Rt 0.750 min.

### Step 2. 1-(2-bromo-6-methyl-4-pyridyl)ethanamine

The mixture of 1-(2-bromo-6-methyl-4-pyridyl)ethanone oxime (3.2 g, 13.97 mmol, 1 eq.) and Zn (3.20 g, 48.89 mmol, 3.5 eq.) in HOAc (20 mL) and H₂O (10 mL) was stirred at room temperature for 1 h. The reaction mixture was filtered and washed with H₂O (3×10 mL). The pH value was adjusted to pH=8 with ammonium hydroxide. The resulting mixture was extracted with EA (3×100 mL). The combined organic layers were washed with brine (2×150 mL), dried over anhydrous Na₂SO₄. After filtration, the filtrate was concentrated to give 1-(2-bromo-6-methyl-4-pyridyl)ethanamine (4 g, crude) as a yellow solid. LCMS (ES, m/z): 215, 217 [M+H]⁺, Rt 0.677 min.

### Step 3. tert-butyl N-[1-(2-bromo-6-methyl-4-pyridyl)ethyl]carbamate

To a solution of 1-(2-bromo-6-methylpyridin-4-yl)ethan-1-amine (2 g, 9.30 mmol, 1 eq.) and triethylamine (4.70 g, 46.49 mmol, 5 eq.) in DCM (20 mL) was added Boc₂O (4.06 g, 18.60 mmol, 2 eq.) at 0° C. The mixture was stirred at room temperature for 12 h. To the above was added water (150 mL). The mixture was extracted with DCM (3×100 mL). The combined organic layers were washed with brine (200 mL), dried over anhydrous Na₂SO₄ and concentrated. The residue was purified by silica gel column chromatography, eluted with EA/PE (1:1) to afford tert-butyl N-[1-(2-bromo-6-methyl-4-pyridyl)ethyl]carbamate (1.4 g, 4.44 mmol, 48% yield) as a yellow solid. LCMS (ES, m/z): 315, 317 [M+H]⁺, Rt 0.710 min.

### Step 4. tert-butyl (1-(2-bromo-6-methylpyridin-4-yl)ethyl)(methyl)carbamate

To a solution of tert-butyl N-[1-(2-bromo-6-methyl-4-pyridyl)ethyl]carbamate (1.4 g, 4.44 mmol, 1 eq.) in DMF (10 mL) and THF (10 mL) was added NaH (60% purity, 355.2 mg, 8.88 mmol, 2 eq.) at 0° C. under the N₂ atmosphere and stirred for 30 min. Then iodomethane (1.89 g, 13.32 mmol, 3 eq.) was added at 0° C. and the reaction was stirred 2 h at room temperature. The reaction mixture was quenched with ice/water (100 mL) and extracted with EA (3×80 mL). The combined organic layers were washed with brine (2×150 mL), dried over anhydrous Na₂SO₄ and concentrated. The residue was purified by silica gel column chromatography, eluted with EA/PE (1:1) to afford tert-butyl (1-(2-bromo-6-methylpyridin-4-yl)ethyl)(methyl)carbamate (1.2 g, 3.65 mmol, 82% yield) as a yellow oil. ¹H NMR (300 MHz, DMSO-d6) δ 7.20 (s, 1H), 7.13 (s, 1H), 5.28-4.91 (m, 1H), 2.64 (s, 3H), 2.42 (s, 3H), 1.42 (d, J=7.2 Hz, 3H), 1.35 (s, 9H). LCMS (ES, m/z): 329, 331 [M+H]⁺, Rt 0.743 min.

### Step 5. tert-butyl (R)-(1-(2-bromo-6-methylpyridin-4-yl)ethyl)(methyl)carbamate and tert-butyl (S)-(1-(2-bromo-6-methylpyridin-4-yl)ethyl)(methyl)carbamate

The racemic tert-butyl (1-(2-bromo-6-methylpyridin-4-yl)ethyl)(methyl) carbamate (1.2 g, 3.65 mmol, 1 eq.) was separated by Chiral-SFC (Column: CHIRAL ART Cellulose-SC, 7*25 cm/10 μm; Mobile Phase A: CO₂, Mobile Phase B: IPA (0.5% 2M NH₃-MeOH); Flow rate: 250 mL/min; Gradient: isocratic 50% B; Column Temperature(° C.): 35; Back Pressure(bar): 100; Wave Length: 220 nm; RT1(min): 8.5; RT2(min): 11) to afford tert-butyl (R)-(1-(2-bromo-6-methylpyridin-4-yl)ethyl)(methyl)carbamate (520 mg, 1.58 mmol, 43% yield) as a yellow oil and tert-butyl (S)-(1-(2-bromo-6-methylpyridin-4-yl)ethyl)(methyl)carbamate (470 mg, 1.43 mmol, 39% yield) as a yellow oil. LCMS (ES, m/z): 329, 331 [M+H]⁺, Rt 0.743 min.

Absolute stereochemistry of title compounds were assigned arbitrarily upon chiral separation.

### Intermediate 213. 1-((6-bromopyridin-2-yl)imino)tetrahydro-1H-1l6-thiophene 1-oxide

### Step 1. 1-iminothiolane 1-oxide

A solution of tetrahydrothiophene (500 mg, 5.67 mmol, 1 eq.), PhI(OAc)₂ (4.57 g, 14.18 mmol, 2.5 eq.) and ammonia carbamic acid (1.11 g, 14.18 mmol, 2.5 eq.) in MeOH (40 mL) was stirred for 3 h at room temperature. The solvent was removed under reduced pressure. The residue was purified by silica gel column chromatography, eluted with DCM/MeOH (10:1) to afford 1-iminothiolane 1-oxide (290 mg, 2.43 mmol, 43% yield) as a colorless oil. ¹H NMR (400 MHz, DMSO-d6) δ 3.79 (br, 1H), 3.02-2.88 (m, 4H), 2.13-1.97 (m, 4H).

### Step 2. 1-((6-bromopyridin-2-yl)imino)tetrahydro-1H-1l6-thiophene 1-oxide

A mixture of 1-iminothiolane 1-oxide (240 mg, 2.01 mmol, 1 eq.), 2,6-dibromopyridine (572.43 mg, 2.42 mmol, 1.2 eq.), Xantphos Pd G2 (178.96 mg, 201.37 μmol, 0.1 eq.), Xantphos (233.03 mg, 402.74 μmol, 0.2 eq.) and sodium tert-butoxide (387.04 mg, 4.03 mmol, 2 eq.) in 1,4-dioxane (10 mL) was stirred for 2 h at 80° C. under N₂ atmosphere. The mixture was allowed to cool down to room temperature, then filtered and the filter cake was washed with DCM (3×10 mL). The filtrate was concentrated and the residue was purified by silica gel column chromatography, eluted with DCM/MeOH (10:1) to afford 1-((6-bromopyridin-2-yl)imino)tetrahydro-1H-1l6-thiophene 1-oxide (340 mg, 1.24 mmol, 62% yield) as a yellow solid. ¹H NMR (400 MHz, DMSO-d6) δ 7.49 (t, J=8.0 Hz, 1H), 7.01 (d, J=7.6 Hz, 1H), 6.70 (d, J=8.0 Hz, 1H), 3.62-3.49 (m, 2H), 3.39-3.27 (m, 2H), 2.28-2.04 (m, 4H). LCMS (ES, m/z): 275, 277 [M+H]⁺, Rt 1.016 min.

### Intermediate 240. 3-fluoro-4-(imidazol-1-yl)aniline

### Step 1. 1-(2-fluoro-4-nitrophenyl)imidazole

To a stirred solution of 1,2-difluoro-4-nitrobenzene (1 g, 6.286 mmol, 1 eq.) and K₂CO₃ (2.61 g, 18.858 mmol, 3 eq.) in DMF (12 mL) was added imidazole (0.86 g, 12.572 mmol, 2 eq.) portionwise at 0° C. The resulting mixture was stirred for 2 h at 100° C. The reaction was quenched by H₂O (100 mL) and the resulting mixture was extracted with EA (3×80 mL). The combined organic layers were washed with brine (150 mL), dried over anhydrous Na₂SO₄. After filtration, the filtrate was concentrated under reduced pressure. The residue was purified by silica gel column chromatography, eluted with [PE:EA](1:1) to afford 1-(2-fluoro-4-nitrophenyl)imidazole (1.15 g, 5.53 mmol, 88%) as a yellow solid. LCMS (ES, m/z): 208 [M+H]⁺, Rt 0.458 min.

### Step 2. 3-fluoro-4-(imidazol-1-yl)aniline

To a solution of 1-(2-fluoro-4-nitrophenyl)imidazole (1.15 g, 5.551 mmol, 1 eq.) in EtOH (20 mL) was added Pd/C (230 mg, 10 wt %). The mixture was hydrogenated at room temperature for 1 h under H₂ atmosphere using a hydrogen balloon. The solids were filtered through a Celite pad and washed by EtOH (3×5 mL). The filtrate was concentrated under reduced pressure. The residue was purified by silica gel column chromatography, eluted with PE:EA (1:3) to afford 3-fluoro-4-(imidazol-1-yl)aniline (830 mg, 4.69 mmol, 84%) as a yellow solid. ¹H NMR (300 MHz, DMSO-d6) δ 7.79 (s, 1H), 7.35 (s, 1H), 7.18 (t, J=9.0 Hz, 1H), 7.07-7.03 (m, 1H), 6.54-6.45 (m, 2H), 5.70 (s, 2H). LCMS (ES, m/z): 178 [M+H]⁺, Rt 0.272 min.

TABLE 12
The following intermediates were prepared in an analogous manner as
Intermediate 240 starting from appropriate nitro-aryl starting material.

| Structure/Intermediate # | LCMS |
|---|---|
| 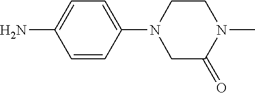 | LCMS (ES, m/z): 206 [M + H]⁺, Rt 0.604 min. |
| Intermediate 241 | |
| 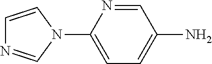 [SMILES: Nc1ccc(-n2ccnc2)nc1] | LCMS (ES, m/z): 161 [M + H]⁺, Rt 0.166 min. |
| Intermediate 242 | |
| 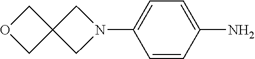 [SMILES: Nc1ccc(N2CC3(COC3)C2)cc1] | LCMS (ES, m/z): 191 [M + H]⁺, Rt 0.375 min. |
| Intermediate 243 | |
| 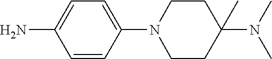 [SMILES: CN(C)C1(C)CCN(c2ccc(N)cc2)CC1] | LCMS (ES, m/z): 234 [M + H]⁺, Rt 0.934 min. |
| Intermediate 246 | |
| 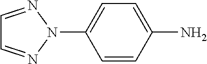 [SMILES: Nc1ccc(-n2nccn2)cc1] | LCMS (ES, m/z): 161 [M + H]⁺, Rt 0.392 min. |
| Intermediate 264 | |
| 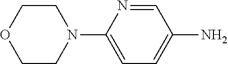 [SMILES: Nc1ccc(N2CCOCC2)nc1] | LCMS (ES, m/z): 180 [M + H]⁺, Rt 0.514 min. |
| Intermediate 267 | |
| 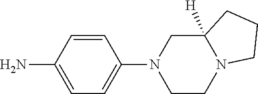 [SMILES: Nc1ccc(N2CCN3CCC[C@@H]3C2)cc1] | LCMS (ES, m/z): 218 [M + H]⁺, Rt 0.170 min. |
| Intermediate 269 | |
| 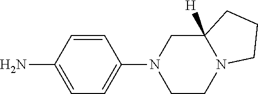 [SMILES: Nc1ccc(N2CCN3CCC[C@H]3C2)cc1] | LCMS (ES, m/z): 218 [M + H]⁺, Rt 0.265 min. |
| Intermediate 270 | |
| 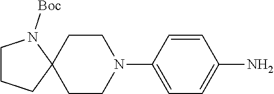 [SMILES: CC(C)(C)OC(=O)N1CCCC12CCN(c1ccc(N)cc1)CC2] | LCMS (ES, m/z): 332 [M + H]⁺, Rt 1.097 min. |
| Intermediate 247 | |
| 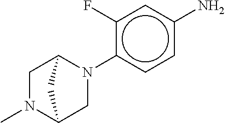 | LCMS (ES, m/z): 222 [M + H]⁺, Rt 0.16 min. |
| Intermediate 58 | |
| 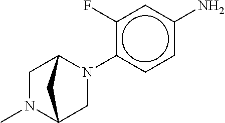 | LCMS (ES, m/z): 222 [M + H]⁺, Rt 0.597 min. |
| Intermediate 59 | |

### Intermediate 244 and Intermediate 245. 4-((5R)-1,4-diazabicyclo[3.2.1]octan-4-yl)aniline and 4-((5S)-1,4-diazabicyclo[3.2.1]octan-4-yl)aniline

### Step 1. 4-(4-nitrophenyl)-1,4-diazabicyclo[3.2.1]octane

Following the experimental procedure described in step 1/Intermediate 240, 4-(4-nitrophenyl)-1,4-diazabicyclo[3.2.1]octane (100 mg, 428.70 μmol, 36% yield) was obtained as a yellow solid, starting from 1-fluoro-4-nitro-benzene (200 mg, 1.42 mmol, 1.2 eq.) and 1,4-diazabicyclo[3.2.1]octane (132.50 mg, 1.18 mmol, 1 eq.). LCMS (ES, m/z): 234 [M+H]⁺, Rt 0.633 min.

### Step 2. (5R)-4-(4-nitrophenyl)-1,4-diazabicyclo[3.2.1]octane and (5S)-4-(4-nitrophenyl)-1,4-diazabicyclo[3.2.1]octane

The racemic 4-(4-nitrophenyl)-1,4-diazabicyclo[3.2.1]octane (100 mg, 428.70 μmol, 1 eq.) was separated by Prep-Chiral-HPLC (Column: CHIRALPAK IF, 2*25 cm/5 μm; Mobile Phase A: Hex (0.5% 2M NH₃-MeOH), Mobile Phase B: EtOH; Flow rate: 16 mL/min; Gradient: 50% B in 35 min; Wave Length: 220/254 nm; RT1(min): 22.362; RT2(min): 30.121) to afford (5R)-4-(4-nitrophenyl)-1,4-diazabicyclo[3.2.1]octane (30 mg, 128.61 μmol, 30% yield) and (5S)-4-(4-nitrophenyl)-1,4-diazabicyclo[3.2.1]octane (30 mg, 128.61 μmol, 30% yield) as yellow solid. LCMS (ES, m/z): 234 [M+H]⁺, Rt 0.633 min.

Absolute stereochemistry of title compounds was arbitrarily assigned upon the separation of racemic mixture by chiral-HPLC.

### Step 3. 4-((5R)-1,4-diazabicyclo[3.2.1]octan-4-yl)aniline and 4-((5S)-1,4-diazabicyclo[3.2.1]octan-4-yl)aniline

Following the experimental procedure described in step 2/intermediate 240, 4-((5R)-1,4-diazabicyclo[3.2.1]octan-4-yl)aniline (20 mg, crude) was obtained as a brown solid, starting from (5R)-4-(4-nitrophenyl)-1,4-diazabicyclo[3.2.1]octane (30 mg, 128.61 μmol, 1 eq.). LCMS (ES, m/z): 204 [M+H]⁺, Rt 0.410 min.

Following the experimental procedure described in step 2/intermediate 240, 4-((5S)-1,4-diazabicyclo[3.2.1]octan-4-yl)aniline (20 mg, crude) was obtained as a brown solid, starting from (5S)-4-(4-nitrophenyl)-1,4-diazabicyclo[3.2.1]octane (30 mg, 128.61 μmol, 1 eq.). LCMS (ES, m/z): 204 [M+H]⁺, Rt 0.410 min.

### Intermediate 248. 3-chloro-4-((1S,4S)-5-methyl-2,5-diazabicyclo[2.2.1]heptan-2-yl)aniline

### Step 1. (1S,4S)-2-(2-chloro-4-nitro-phenyl)-5-methyl-2,5-diazabicyclo[2.2.1]heptane

To a stirred mixture of 2-chloro-1-fluoro-4-nitro-benzene (1 g, 5.70 mmol, 1 eq.) and (1S,4S)-2-methyl-2,5-diazabicyclo[2.2.1]heptane (639.00 mg, 5.70 mmol, 1 eq.) in DMF (15 mL) was added K₂CO₃ (2.36 g, 17.09 mmol, 3 eq.) in portions. The resulting mixture was stirred for 2 h at 100° C. The reaction was allowed to cool down to room temperature and was diluted with H₂O (100 mL). The mixture was extracted with EA (3×80 mL). The combined organic layers were washed with brine (2×150 mL), dried over anhydrous Na₂SO₄. After filtration, the filtrate was concentrated under reduced pressure. The residue was purified by silica gel column chromatography, eluted with EA/PE (1:1) to afford (1S,4S)-2-(2-chloro-4-nitro-phenyl)-5-methyl-2,5-diazabicyclo[2.2.1]heptane (1.2 g, 4.48 mmol, 78% yield) as a yellow solid. ¹H NMR (300 MHz, DMSO-d₆) δ 8.10 (d, J=2.7 Hz, 1H), 8.03-7.93 (m, 1H), 6.92 (d, J=9.3 Hz, 1H), 4.63-4.60 (m, 1H), 3.90-3.79 (m, 1H), 3.52-3.42 (m, 2H), 2.90-2.80 (m, 1H), 2.72-2.62 (m, 1H), 2.27 (s, 3H), 1.96-1.86 (m, 1H), 1.82-1.72 (m, 1H). LCMS (ES, m/z): 268, 270 [M+H]⁺, Rt 0.468 min.

### Step 2. 3-chloro-4-((1S,4S)-5-methyl-2,5-diazabicyclo[2.2.1]heptan-2-yl)aniline

To a stirred mixture of (1S,4S)-2-(2-chloro-4-nitro-phenyl)-5-methyl-2,5-diazabicyclo[2.2.1]heptane (1.10 g, 4.11 mmol, 1 eq.) and NH₄Cl (2.20 g, 41.09 mmol, 10 eq.) in EtOH (10 mL) and H₂O (2 mL) was added Zn (1.61 g, 24.65 mmol, 6 eq.) in portions. The resulting mixture was stirred for 1 h at 80° C. under the N₂ atmosphere. The reaction was allowed to cool down to room temperature. The resulting mixture was filtered, the filter cake was washed with EtOH (3×10 mL). The filtrate was concentrated under reduced pressure to afford 3-chloro-4-[(1S,4S)-5-methyl-2,5-diazabicyclo[2.2.1]heptan-2-yl]aniline (1 g, crude) as a brown solid. LCMS (ES, m/z): 238, 240 [M+H]⁺, Rt 0.589 min.

### Intermediate 249. 3-chloro-4-((1R,4R)-5-methyl-2,5-diazabicyclo[2.2.1]heptan-2-yl)aniline

Following the general procedures described for Intermediate 248, 3-chloro-4-((1R,4R)-5-methyl-2,5-diazabicyclo[2.2.1]heptan-2-yl)aniline (1 g, crude) was obtained as a yellow oil, starting from 2-chloro-1-fluoro-4-nitro-benzene (1 g, 5.70 mmol, 1 eq.) and (1R,4R)-2-methyl-2,5-diazabicyclo[2.2.1]heptane (639.00 mg, 5.70 mmol, 1 eq.). ¹H NMR (400 MHz, DMSO-d6) δ 6.92-6.89 (m, 1H), 6.62 (d, J=2.8 Hz, 1H), 6.46 (dd, J=8.4, 2.4 Hz, 1H), 4.93 (br, 2H), 4.00-3.91 (m, 2H), 3.48-3.46 (m, 1H), 3.35-3.31 (m, 1H), 2.98-2.90 (m, 1H), 2.61 (s, 3H), 2.08-2.01 (m, 1H), 1.91-1.89 (m, 2H). LCMS (ES, m/z): 238, 240 [M+H]⁺, Rt 0.258 min.

### Intermediate 250 and Intermediate 251. tert-butyl (3R,4R)-3-(4-aminophenyl)-4-fluoropyrrolidine-1-carboxylate and tert-butyl (3S,4S)-3-(4-aminophenyl)-4-fluoropyrrolidine-1-carboxylate

### Step 1. rac-tert-butyl (3R,4S)-3-(4-bromophenyl)-4-hydroxy-pyrrolidine-1-carboxylate

To a stirred solution of 1,4-dibromobenzene (8.92 g, 37.79 mmol, 1 eq.) and boron trifluoride etherate (5.36 g, 37.79 mmol, 1 eq.) in THF (50 mL) was added dropwise n-BuLi (2.2M in hexane, 25.4 mL, 56.69 mmol, 1.5 eq,) at −78° C. under N₂ atmosphere. The reaction mixture was stirred at −78° C. for 30 min. To the above mixture was added tert-butyl 6-oxa-3-azabicyclo[3.1.0]hexane-3-carboxylate (7 g, 37.79 mmol, 1 eq.) in THF (10 mL) dropwise. The mixture was stirred for another 1.5 h at −78° C. The reaction was quenched by ice/water (150 mL), and then the mixture was extracted with EA (2×100 mL). The combined organic layers were washed with brine (200 mL), dried over anhydrous Na₂SO₄, filtered and concentrated under vacuum. The residue was purified by silica gel column chromatography, eluted with EA/PE (2:1) to afford rac-tert-butyl (3R,4S)-3-(4-bromophenyl)-4-hydroxy-pyrrolidine-1-carboxylate (4.0 g, 10.52 mmol, 28% yield) as a yellow oil. LCMS (ES, m/z): 286, 288 [M+H-56]⁺, Rt 1.389 min.

### Step 2. rac-tert-butyl (3R,4R)-3-(4-bromophenyl)-4-fluoropyrrolidine-1-carboxylate

To a stirred solution of rac-tert-butyl (3R,4S)-3-(4-bromophenyl)-4-hydroxy-pyrrolidine-1-carboxylate (600 mg, 1.75 mmol, 1 eq) in DCM (15 mL) was added DAST (847.80 mg, 5.26 mmol, 3 eq.) dropwise at −60° C. under N₂ atmosphere. And then the resulting mixture was stirred for 2 h at room temperature under the N₂ atmosphere. The reaction was quenched by the addition of saturated NaHCO₃ solution at 0° C. The resulting mixture was extracted with DCM (3×100 mL). The combined organic layers were washed with brine (2×100 mL), dried over anhydrous MgSO₄, filtered and concentrated under vacuum. The residue was purified by silica gel column chromatography, eluted with EA/PE (1:3) to afford rac-tert-butyl (3R,4R)-3-(4-bromophenyl)-4-fluoropyrrolidine-1-carboxylate (430 mg, 1.12 mmol, 64.13% yield) as a yellow oil. LCMS (ES, m/z): 344, 346 [M+H]⁺, Rt 1.145 min.

### Step 3. rac-tert-butyl (3R,4R)-3-(4-((diphenylmethylene)amino)phenyl)-4-fluoropyrrolidine-1-carboxylate

To a stirred mixture of rac-tert-butyl (3R,4R)-3-(4-bromophenyl)-4-fluoro-pyrrolidine-1-carboxylate (400 mg, 1.16 mmol, 1 eq.), Brettphos (62.29 mg, 0.12 mmol, 0.1 eq.), diphenylmethanimine (252.72 mg, 1.39 mmol, 1.2 eq.) and Cs₂CO₃ (1.14 g, 3.49 mmol, 3 eq.) in 1,4-dioxane (6 mL) was added Brettphos-Pd-G3 (105.28 mg, 116.21 μmol, 0.1 eq) in portions under N₂ atmosphere. The resulting mixture was stirred for 3 h at 100° C. under the N₂ atmosphere. The reaction was allowed to cool down to room temperature. The resulting mixture was filtered, the filter cake was washed with DCM (5×10 mL). The filtrate was concentrated under reduced pressure. The residue was purified by silica gel column chromatography, eluted with EA/PE (1:5) to afford rac-tert-butyl (3R,4R)-3-(4-((diphenylmethylene)amino)phenyl)-4-fluoropyrrolidine-1-carboxylate (300 mg, 0.67 mmol, 58% yield) as a yellow oil. LCMS (ES, m/z): 445 [M+H]⁺, Rt 1.213 min.

### Step 4. rac-tert-butyl (3R,4R)-3-(4-aminophenyl)-4-fluoro-pyrrolidine-1-carboxylate

A solution of rac-tert-butyl (3R,4R)-3-(4-((diphenylmethylene)amino)phenyl)-4-fluoropyrrolidine-1-carboxylate (270 mg, 607.37 μmol, 1 eq.) in AcOH (5 mL) was stirred for 1 h at 100° C. The resulting mixture was extracted with EA (3×50 mL) upon cooling to room temperature. The combined organic layers were washed with brine (2×50 mL), dried over anhydrous Na₂SO₄. After filtration, the filtrate was concentrated under reduced pressure. The residue was purified by silica gel column chromatography, eluted with EA/PE (1:1) to afford rac-tert-butyl (3R,4R)-3-(4-aminophenyl)-4-fluoro-pyrrolidine-1-carboxylate (110 mg, 391.99 μmol, 65% yield) as a yellow oil. ¹H NMR (400 MHz, DMSO-d6) δ 6.88 (d, J=8.4 Hz, 2H), 6.53 (d, J=8.4 Hz, 2H), 5.02 (s, 2H), 3.69-3.66 (m, 1H), 3.61-3.33 (m, 5H), 1.43 (s, 9H). LCMS (ES, m/z): 281 [M+H]⁺, Rt 0.698 min.

### Step 5. tert-butyl (3R,4R)-3-(4-aminophenyl)-4-fluoropyrrolidine-1-carboxylate and tert-butyl (3S,4S)-3-(4-aminophenyl)-4-fluoropyrrolidine-1-carboxylate

Rac-tert-butyl (3R,4R)-3-(4-aminophenyl)-4-fluoro-pyrrolidine-1-carboxylate (420 mg, 1.50 mmol, 1 eq.) was separated by Chiral-HPLC: (Column: CHIRAL ART Cellulose-SC, 2*25 cm/5 μm; Mobile Phase A: Hex (0.5% 2M NH₃-MeOH), Mobile Phase B: EtOH; Flow rate: 20 mL/min; Gradient: 15% B in 14 min; Wave Length: 254/220 nm; RT1(min): 7.99; RT2(min): 11.12) to afford tert-butyl (3R,4R)-3-(4-aminophenyl)-4-fluoropyrrolidine-1-carboxylate (170 mg, 41% yield) and tert-butyl (3S,4S)-3-(4-aminophenyl)-4-fluoropyrrolidine-1-carboxylate (170 mg, 41% yield) as a yellow solids. LCMS (ES, m/z): 281 [M+H]⁺, Rt 0.698 min.

Absolute stereochemistry of title compounds was arbitrarily assigned upon the separation of racemic mixture by chiral-HPLC.

### Intermediate 252 and Intermediate 253. tert-butyl (R)-4-(4-aminophenyl)-3,3-difluoropyrrolidine-1-carboxylate

### And tert-butyl (S)-4-(4-aminophenyl)-3,3-difluoropyrrolidine-1-carboxylate

### Step 1. tert-butyl 3-(4-bromophenyl)-4-oxopyrrolidine-1-carboxylate

To a stirred solution of rac-tert-butyl (3R,4S)-3-(4-bromophenyl)-4-hydroxy-pyrrolidine-1-carboxylate (step 1/intermediate 250/251, 4 g, 11.688 mmol, 1 eq.) in DCM (50 mL) was added Dess-Martin periodinane (9.91 g, 23.376 mmol, 2 eq.) in portions at 0° C. The resulting mixture was stirred for 16 h at room temperature. The reaction was quenched with Na₂S₂O₃/NaHCO₃ (200 mL/200 mL) at 0° C. The mixture was extracted with EA (3×300 mL), the combined organic layers were washed with brine (2×300 mL), dried over anhydrous Na₂SO₄. After filtration, the filtrate was concentrated under reduced pressure and residue was purified by silica gel column chromatography, eluted with PE:EA (1:1) to afford tert-butyl 3-(4-bromophenyl)-4-oxopyrrolidine-1-carboxylate (2.5 g, 7.35 mmol, 63%) as a yellow solid. LCMS (ES, m/z): 284, 342 [M+H-56]⁺, Rt 1.085 min.

### Step 2. tert-butyl 4-(4-bromophenyl)-3,3-difluoropyrrolidine-1-carboxylate

To a solution of tert-butyl 3-(4-bromophenyl)-4-oxopyrrolidine-1-carboxylate (2.5 g, 7.348 mmol, 1 eq.) in DCM (50 mL) was added DAST (23.69 g, 146.960 mmol, 20 eq.) at −60° C. under N₂ atmosphere. The resulting mixture was stirred for 6 h at room temperature. The reaction was quenched by ice/H₂O (200 mL) and the resulting mixture was extracted with EA (3×100 mL). The combined organic layers were washed with brine (2×200 mL), dried over anhydrous Na₂SO₄. After filtration, the filtrate was concentrated under reduced pressure. The residue was purified by silica gel column chromatography, eluted with PE:EA (1:2) to afford tert-butyl 4-(4-bromophenyl)-3,3-difluoropyrrolidine-1-carboxylate (690 mg, 1.91 mmol, 26%) as a yellow solid. ¹H NMR (400 MHz, DMSO-d6) δ 7.60 (d, J=8.4 Hz, 2H), 7.33 (d, J=8.4 Hz, 2H), 4.06-3.88 (m, 2H), 3.84-3.75 (m, 2H), 3.66-3.57 (m, 1H), 1.44 (s, 9H). LCMS (ES, m/z): 306, 308 [M+H-56]⁺, Rt 1.194 min.

### Step 3-4. tert-butyl 4-(4-aminophenyl)-3,3-difluoropyrrolidine-1-carboxylate

Following the experimental procedures (steps 3-4) described for Intermediate 250/251, the title compound (190 mg, 0.64 mmol, 33% overall yield) was obtained as a yellow oil, starting from tert-butyl 4-(4-bromophenyl)-3,3-difluoropyrrolidine-1-carboxylate (690 mg, 1.91 mmol, 1 eq.) and diphenylmethanimine (415 mg, 2.29 mmol). LCMS (ES, m/z): 299 [M+H]⁺, Rt 0.743 min.

### Step 5. tert-butyl (R)-4-(4-aminophenyl)-3,3-difluoropyrrolidine-1-carboxylate

### And tert-butyl (S)-4-(4-aminophenyl)-3,3-difluoropyrrolidine-1-carboxylate

Tert-butyl 4-(4-aminophenyl)-3,3-difluoropyrrolidine-1-carboxylate (260 mg, 0.87 mmol, 1 eq) was in separated by Chiral-HPLC (Column: CHIRAL ART Cellulose-SC, 2*25 cm/5 μm; Mobile Phase A: Hex (0.5% 2M NH₃-MeOH), Mobile Phase B: EtOH; Flow rate: 20 mL/min; Gradient: 20% B in 14 min; Wave Length: 254/220 nm; RT1(min): 7.99; RT2(min): 11.12) to afford tert-butyl (R)-4-(4-aminophenyl)-3,3-difluoropyrrolidine-1-carboxylate (100 mg, 38%) as a yellow solid and tert-butyl (S)-4-(4-aminophenyl)-3,3-difluoropyrrolidine-1-carboxylate (120 mg, 46%) as a yellow solid. LCMS (ES, m/z): 299 [M+H]⁺, Rt 0.743 min. Absolute stereochemistry of title compounds was arbitrarily assigned upon the separation of racemic mixture by chiral-HPLC.

### Intermediate 254. tert-butyl 2-(4-aminophenyl)-2-methyl-propanoate

### Step 1. tert-butyl 2-(4-nitrophenyl)acetate

To a solution of 2-(4-nitrophenyl)acetic acid (2 g, 11.04 mmol, 1 eq.) and 2-methylpropan-2-ol (4.09 g, 55.20 mmol, 5 eq.) in phosphoryl trichloride (15 mL) were added pyridine (2.62 g, 33.12 mmol, 3 eq). After stirring for 16 h at room temperature, the resulting mixture was concentrated under reduced pressure. The residue was purified by silica gel column chromatography, eluted with PE/EA (2:1) to afford tert-butyl 2-(4-nitrophenyl)acetate (2 g, 8.35 mmol, 76% yield) as a yellow oil. LCMS (ES, m/z): 238 [M+H]⁺, Rt 1.025 min.

### Step 2. tert-butyl 2-methyl-2-(4-nitrophenyl)propanoate

To a solution of tert-butyl 2-(4-nitrophenyl)acetate (2 g, 8.43 mmol, 1 eq.) in DMF (20 mL) was added NaH (60% in mineral oil, 1.01 g, 25.29 mmol, 3 eq.) in portions at 0° C. The mixture was stirred for 30 mins at room temperature. Then Mel (3.59 g, 25.29 mmol, 3 eq.) was added and the mixture was allowed to warm to room temperature and stirred for an additional 2 h. The reaction was quenched by ice/water (150 mL) and extracted with EA (3×100 mL). The combined organic layers were washed with brine (2×150 mL), dried over anhydrous Na₂SO₄. After filtration, the filtrate was concentrated under reduced pressure. The residue was purified by silica gel column chromatography, eluted with EA/PE (1:1) to afford tert-butyl 2-methyl-2-(4-nitrophenyl)propanoate (1.7 g, 5.96 mmol, 71% yield) as a yellow oil. LCMS (ES, m/z): 266 [M+H]⁺, Rt 1.345 min.

### Step 3. tert-butyl 2-(4-aminophenyl)-2-methyl-propanoate

To a solution of tert-butyl 2-methyl-2-(4-nitrophenyl)propanoate (1.7 g, 6.41 mmol, 1 eq.) in EtOH (30 mL) was added Pd/C (170 mg, 10 wt %). The mixture was hydrogenated at room temperature for 2 h under the hydrogen atmosphere using a hydrogen balloon. The reaction was filtered and the filter cake was washed with EtOH (3×10 mL). The filtrate was concentrated under reduced pressure. The residue was purified by silica gel column chromatography, eluted with EA/PE (1:1) to afford tert-butyl 2-(4-aminophenyl)-2-methyl-propanoate (1.20 g, 4.84 mmol, 76% yield) as a yellow solid. ¹H NMR (300 MHz, DMSO-d6) δ 6.98 (d, J=8.7 Hz, 2H), 6.52 (d, J=8.7 Hz, 2H), 4.95 (s, 2H), 1.37 (s, 6H), 1.33 (s, 9H). LCMS (ES, m/z): 236 [M+H]⁺, Rt 0.744 min.

### Intermediate 256. 5-(4-methylpiperazin-1-yl)pyridin-3-amine

### Step 1. 1-methyl-4-(5-nitropyridin-3-yl)piperazine

The mixture of 3-bromo-5-nitropyridine (1 g, 4.93 mmol, 1 eq.), 1-methylpiperazine (1.73 g, 17.24 mmol, 3.5 eq.), Cs₂CO₃ (3.21 g, 9.85 mmol, 2 eq.), Pd₂(dba)₃ (225.55 mg, 246.31 umol, 0.05 eq.) and Xantphos (285.04 mg, 492.63 umol, 0.1 eq.) in 1,4-dioxane (12 mL) was stirred at 100° C. for 12 h under N₂ atmosphere. The reaction was allowed to cool down to room temperature and diluted with water (100 mL). The mixture was extracted with EA (3×80 mL). The combined organic layers were washed with brine (2×150 mL) and dried over anhydrous Na₂SO₄. After filtration, the filtrate was concentrated under reduced pressure. The residue was purified by silica gel column chromatography, eluted with EA/PE (1:1) to afford 1-methyl-4-(5-nitropyridin-3-yl)piperazine (1 g, 4.50 mmol, 91% yield) as a yellow solid. LCMS (ES, m/z): 223 [M+H]⁺, Rt 0.358 min.

### Step 2. 5-(4-methylpiperazin-1-yl)pyridin-3-amine

To a solution of 1-methyl-4-(5-nitropyridin-3-yl)piperazine (2.6 g, 11.70 mmol, 1 eq.) in MeOH (25 mL) was added Pd/C (260 mg, 10 wt %) in portions. The reaction was stirred at room temperature for 4 h under H₂ atmosphere (1 atm). The resulting mixture was filtered, the filter cake was washed with MeOH (3×10 mL). The filtrate was concentrated and purified by silica gel column chromatography, eluted with MeOH/DCM (1:10) to afford 5-(4-methylpiperazin-1-yl)pyridin-3-amine (2 g, 10.40 mmol, 89% yield) as a yellow solid. LCMS (ES, m/z): 193 [M+H]⁺, Rt 0.187 min.

### Intermediate 259. tert-butyl 4-(5-aminopyridin-3-yl)piperidine-1-carboxylate

### Step 1. tert-butyl 4-(5-nitro-3-pyridyl)-3,6-dihydro-2H-pyridine-1-carboxylate

To a stirred mixture of 3-chloro-5-nitro-pyridine (500 mg, 3.15 mmol, 1 eq.) and tert-butyl 4-(4,4,5,5-tetramethyl-1,3,2-dioxaborolan-2-yl)-3,6-dihydro-2H-pyridine-1-carboxylate (1.17 g, 3.78 mmol, 1.2 eq.) in 1,4-dioxane (8 mL) and H₂O (2 mL) were added Pd(PPh₃)₂Cl₂ (221.36 mg, 315.37 μmol, 0.1 eq.) and K₂CO₃ (1.09 g, 7.88 mmol, 2.5 eq.) at room temperature. The reaction mixture was stirred for 2 h at 90° C. under the N₂ atmosphere. The mixture was allowed to cool down to room temperature and was filtered. The filter cake was washed with EA (3×10 mL) and filtrate was concentrated under reduced pressure. The residue was purified by silica gel column chromatography, eluted with EA/PE (1:5) to afford tert-butyl 4-(5-nitro-3-pyridyl)-3,6-dihydro-2H-pyridine-1-carboxylate (690 mg, 2.26 mmol, 72% yield) as a brown solid. ¹H NMR (300 MHz, DMSO-d₆) δ 9.25 (s, 1H), 9.08 (s, 1H), 8.53 (s, 1H), 6.61-6.45 (m, 1H), 4.10-4.04 (m, 2H), 3.62-3.51 (m, 2H), 2.64-2.53 (m, 2H), 1.44 (s, 9H). LCMS (ES, m/z): 306 [M+H]⁺, Rt 0.762 min.

### Step 2. tert-butyl 4-(5-aminopyridin-3-yl)piperidine-1-carboxylate

To a stirred mixture of tert-butyl 4-(5-nitro-3-pyridyl)-3,6-dihydro-2H-pyridine-1-carboxylate (500 mg, 1.64 mmol, 1 eq.) in MeOH (10 mL) was added Pd/C (50 mg, 10 wt %). The resulting mixture was stirred for 2 h at room temperature under the H₂ atmosphere (1 atm). The reaction mixture was filtered, the filter cake was washed with MeOH (3×10 mL). The filtrate was concentrated under reduced pressure. The residue was purified by silica gel column chromatography (eluting with 1:2 EA/PE) to afford tert-butyl 4-(5-aminopyridin-3-yl)piperidine-1-carboxylate (380 mg, 1.38 mmol, 84% yield) as a brown solid. LCMS (ES, m/z): 278 [M+H]⁺, Rt 0.558 min.

### Intermediate 263. 5-fluoro-1-methyl-pyrazol-4-amine

### Step 1. tert-butyl N-(1-methylpyrazol-4-yl)carbamate

To a stirred solution of 1-methylpyrazol-4-amine (2 g, 20.59 mmol, 1 eq.) and TEA (4.17 g, 41.19 mmol, 2 eq.) in DCM (50 mL) was added Boc₂O (6.74 g, 30.89 mmol, 1.5 eq.) in portions at 0° C. The resulting mixture was stirred for 16 h at 25° C. and then was concentrated under reduced pressure. The residue was purified by silica gel column chromatography (eluting with 1:3 EA/PE) to afford tert-butyl N-(1-methylpyrazol-4-yl)carbamate (3 g, 15.21 mmol, 74% yield) as a white solid. LCMS (ES, m/z): 198 [M+H]⁺, Rt 0.831 min.

### Step 2. tert-butyl N-(5-fluoro-1-methyl-pyrazol-4-yl)carbamate

To a stirred solution of tert-butyl N-(1-methylpyrazol-4-yl)carbamate (1 g, 5.07 mmol, 1 eq.) in DCM (20 mL) was added 1-(chloromethyl)-4-fluoro-1,4-diazoniabicyclo[2.2.2]octane ditetrafluoroborate (Selectfluor, 2.69 g, 7.61 mmol, 1.5 eq.) in portions at 0° C. The resulting mixture was stirred for 6 h at 20° C. and then was concentrated under reduced pressure. The residue was purified by silica gel column chromatography (eluting with 1:3 EA/PE) to afford tert-butyl N-(5-fluoro-1-methyl-pyrazol-4-yl)carbamate (160 mg, 743.41 μmol, 15% yield) as a yellow solid. ¹H NMR (400 MHz, CD₃OD) δ 9.80 (s, 1H), 8.45 (s, 1H), 3.34 (s, 3H), 2.31 (s, 9H). LCMS (ES, m/z): 216 [M+H]⁺, Rt 0.627 min.

### Step 3. 5-fluoro-1-methyl-pyrazol-4-amine

A solution of tert-butyl N-(5-fluoro-1-methyl-pyrazol-4-yl)carbamate (150 mg, 696.95 μmol, 1 eq.) in TFA (1 mL) and DCM (3 mL) was stirred for 4 h at 20° C. The mixture was concentrated under reduced pressure to afford 5-fluoro-1-methyl-pyrazol-4-amine (80 mg, crude TFA salt) as a yellow solid. LCMS (ES, m/z): 116 [M+H]⁺, Rt 0.136 min.

### Intermediate 277. (S)-1-methylpyrrolidin-3-yl carbonochloridate

To a stirred mixture of (3S)-1-methylpyrrolidin-3-ol (50 mg, 494.33 μmol, 1 eq.) in THF (6 mL) was added bis(trichloromethyl) carbonate (220.04 mg, 741.50 μmol, 1.5 eq.) at 0° C. The resulting mixture was stirred for 16 h at 30° C. and then was concentrated under reduced pressure to afford (S)-1-methylpyrrolidin-3-yl carbonochloridate (70 mg, crude) as a yellow solid. LCMS (ES, m/z): 164, 166 [M+H]⁺, Rt 0.547 min.

### Intermediate 290. ethyl (E)-5-[tert-butyl(dimethyl)silyl]oxy-4-(4-chloro-2-methylsulfanyl-pyrimidin-5-yl)-4-methyl-pent-2-enoate

To a stirred solution of 3-[tert-butyl(dimethyl)silyl]oxy-2-(4-chloro-2-methylsulfanyl-pyrimidin-5-yl)-2-methyl-propanal (Intermediate 1/step 6, 2.5 g, 6.93 mmol, 1 eq.) in THF (30 mL) was added NaH (60% in mineral oil, 318.44 mg, 13.85 mmol, 2 eq.) at 0° C. The mixture was stirred for 30 mins. To the above was added ethyl 2-diethoxyphosphorylacetate (1.86 g, 8.31 mmol, 1.2 eq) and the mixture was allowed to warm to room temperature and stirred for 2.5 h under N₂ atmosphere. The reaction mixture was quenched by ice/water (100 mL) and extracted with EA (3×100 mL). The combined organic layers were washed with brine (2×200 mL), dried over anhydrous Na₂SO₄. After filtration, the filtrate was concentrated under reduced pressure. The residue was purified by silica gel column chromatography, eluted with EA/PE (1:2) to afford ethyl (E)-5-[tert-butyl(dimethyl)silyl]oxy-4-(4-chloro-2-methylsulfanyl-pyrimidin-5-yl)-4-methyl-pent-2-enoate (2.25 g, 5.22 mmol, 75% yield) as a colorless oil. LCMS (ES, m/z): 431 [M+H]⁺, Rt 1.375 min.

### Intermediate 291. tert-butyl ((6-aminopyridin-2-yl)methyl)(methyl)carbamate

To a stirred solution of tert-butyl ((6-bromopyridin-2-yl)methyl)(methyl)carbamate (Intermediate 2, 500 mg, 1.66 mmol, 1 eq.) in ethylene glycol (5 mL) was added K₂CO₃ (45.89 mg, 332.03 μmol, 0.2 eq.), TMEDA (19.29 mg, 166.01 μmol, 0.1 eq.), Cu₂O (40.95 mg, 83.01 μmol, 0.05 eq.) and ammonium hydroxide (28% solution, 1.5 mL). The resulting mixture was stirred at 80° C. for 16 h under the N₂ atmosphere. The reaction was quenched with water (80 mL) and extracted with EA (3×50 mL). The combined organic layers were washed with brine (100 mL), dried over anhydrous Na₂SO₄. After filtration, the filtrate was concentrated under reduced pressure. The residue was purified by silica gel column chromatography, eluted with MeOH/DCM (1:10) to afford tert-butyl ((6-aminopyridin-2-yl)methyl)(methyl)carbamate (200 mg, 51% yield) as a brown solid. LCMS (ES, m/z): 238 [M+H]⁺, Rt 0.666 min.

### Examples 1 and 2: (4bS,8aS)-4b,7,7-trimethyl-9-(6-((methylamino)methyl)pyridin-2-yl)-N-(4-(piperidin-1-yl)phenyl)-4b,5,7,8,8a,9-hexahydropyrano[3′,4′:4,5]pyrrolo[2,3-d]pyrimidin-2-amine

### (4bR,8aR)-4b,7,7-trimethyl-9-(6-((methylamino)methyl)pyridin-2-yl)-N-(4-(piperidin-1-yl)phenyl)-4b,5,7,8,8a,9-hexahydropyrano[3′,4′:4,5]pyrrolo[2,3-d]pyrimidin-2-amine

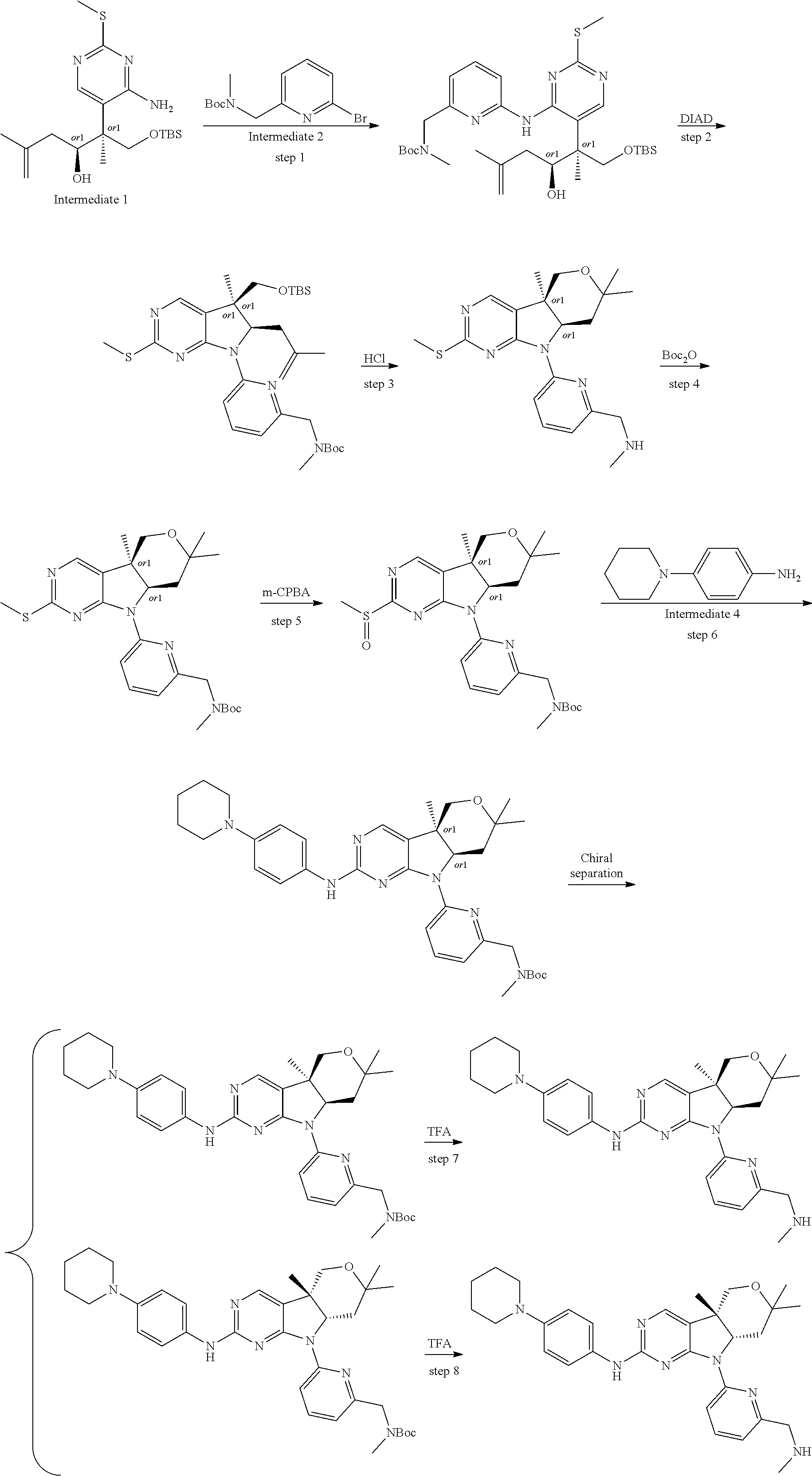

### Step 1. rel-tert-butyl-((6-((5-((2S,3S)-1-((tert-butyldimethylsilyl)oxy)-3-hydroxy-2,5-dimethylhex-5-en-2-yl)-2-(methylthio)pyrimidin-4-yl)amino)pyridin-2-yl)methyl)(methyl)carbamate

A mixture of rel-(2S,3S)-2-(4-amino-2-(methylthio)pyrimidin-5-yl)-1-((tert-butyldimethylsilyl)oxy)-2,5-dimethylhex-5-en-3-ol (Intermediate 1, 1 g, 2.51 mmol, 1 eq.), BINAP Pd G2 (235 mg, 0.25 mmol, 0.1 eq.), Intermediate 2 (909 mg, 3.02 mmol, 1.2 eq.), BINAP (164 mg, 0.25 mmol, 0.1 eq.) and Cs₂CO₃ (1.64 g, 5.03 mmol, 2 eq.) in dioxane (10 mL) was stirred for 2 h at 100° C. under nitrogen atmosphere. The reaction was monitored by LC-MS. The mixture was allowed to cool down to room temperature. After filtration, the filtrate was concentrated under reduced pressure. The residue was purified by silica gel column chromatography (eluting with 1:3 EA/PE) to afford rel-tert-butyl-((6-((5-((2S,3S)-1-((tert-butyldimethylsilyl)oxy)-3-hydroxy-2,5-dimethylhex-5-en-2-yl)-2-(methylthio)pyrimidin-4-yl)amino)pyridin-2-yl)methyl)(methyl)carbamate (1.35 g, 87% yield) as a white solid. LCMS (ES, m/z): 618[M+H]⁺, Rt 0.892 min.

### Step 2. rel-tert-butyl-((6-((5R,6R)-5-(((tert-butyldimethylsilyl)oxy)methyl)-5-methyl-6-(2-methylallyl)-2-(methylthio)-5,6-dihydro-7H-pyrrolo[2,3-d]pyrimidin-7-yl)pyridin-2-yl)methyl)(methyl)carbamate

To a stirred solution of rel-tert-butyl-((6-((5-((2S,3S)-1-((tert-butyldimethylsilyl)oxy)-3-hydroxy-2,5-dimethylhex-5-en-2-yl)-2-(methylthio)pyrimidin-4-yl)amino)pyridin-2-yl)methyl)(methyl)carbamate (1.35 g, 2.18 mmol, 1 eq.) and PPh₃ (1.15 g, 4.37 mmol, 2 eq.) in THF (20 mL) was added DIAD (4.37 mmol, 0.9 mL, 2 eq.) dropwise at 0° C. under nitrogen atmosphere. The resulting mixture was stirred for 2 h at 50° C. under the nitrogen atmosphere. The reaction was monitored by TLC. The resulting mixture was cooled to room temperature and concentrated under reduced pressure. The residue was purified by silica gel column chromatography (eluting with 1:3 EA/PE) to afford rel-tert-butyl-((6-((5R,6R)-5-(((tert-butyldimethylsilyl)oxy)methyl)-5-methyl-6-(2-methylallyl)-2-(methylthio)-5,6-dihydro-7H-pyrrolo[2,3-d]pyrimidin-7-yl)pyridin-2-yl)methyl)(methyl)carbamate (1.15 g, 88% yield) as a white solid. LCMS (ES, m/z): 600[M+H]⁺, Rt 0.954 min.

### Step 3. rel-N-methyl-1-(6-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)methanamine

To a stirred solution of rel-tert-butyl-((6-((5R,6R)-5-(((tert-butyldimethylsilyl)oxy)methyl)-5-methyl-6-(2-methylallyl)-2-(methylthio)-5,6-dihydro-7H-pyrrolo[2,3-d]pyrimidin-7-yl)pyridin-2-yl)methyl)(methyl)carbamate (1.15 g, 1.92 mmol, 1 eq.) in dioxane (10 mL) was added HCl (4 M in dioxane, 10 mL) dropwise at 0° C. The resulting mixture was stirred at r.t. for 3 h. The reaction was monitored by LC-MS. The resulting mixture was concentrated under reduced pressure to afford rel-N-methyl-1-(6-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)methanamine (1.4 g, crude) as a white solid. LCMS (ES, m/z): 386 [M+H]⁺, Rt 0.665 min.

### Step 4. tert-butyl rel-methyl((6-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)methyl)carbamate

To a stirred solution of rel-N-methyl-1-(6-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)methanamine (1.4 g, 1.82 mmol, 50% purity, 1 eq.) in DCM (20 mL) was added DIEA (9.08 mmol, 1.27 mL, 5 eq.) and tert-butoxycarbonyl tert-butyl carbonate (3.63 mmol, 833 uL, 2 eq.) dropwise at 0° C. The resulting mixture was stirred at r.t. for 2 h. The reaction was monitored by LC-MS. The resulting mixture was concentrated under vacuum. The residue was purified by silica gel column chromatography (eluting with 1:3 EA/PE) to afford tert-butyl rel-methyl((6-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)methyl)carbamate (0.8 g, 45% yield) as a white solid. ¹H NMR (300 MHz, DMSO-d6) δ 8.38 (d, J=8.1 Hz, 1H), 8.21 (s, 1H), 7.87-7.82 (t, J=7.8 Hz, 1H), 6.92 (d, J=7.5 Hz, 1H), 4.73-4.68 (m, 1H), 4.42 (s, 2H), 3.88-3.80 (m, 1H), 3.72-3.64 (m, 1H), 2.96-2.92 (m, 3H), 2.51 (s, 3H), 2.29-2.12 (m, 1H), 1.73-1.50 (m, 1H), 1.43 (s, 3H), 1.27 (s, 9H), 1.19 (s, 3H), 0.99 (s, 3H). LCMS (ES, m/z): 486 [M+H]⁺, Rt 0.785 min.

### Step 5. tert-butyl rel-methyl((6-((4bR,8aR)-4b,7,7-trimethyl-2-(methylsulfinyl)-4b,7,8,8a-tetrahydropyrano[3′,4′:4,5]pyrrolo[2,3-d]pyrimidin-9(5H)-yl)pyridin-2-yl)methyl)carbamate

To a stirred solution of tert-butyl rel-methyl((6-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)methyl)carbamate (40 mg, 82.37 umol, 1 eq.) in DCM (1 mL) was added m-CPBA (19 mg, 0.11 mmol, 1.3 eq.) in portions. The resulting mixture was stirred at 0° C. for 1 h. The reaction was monitored by LC-MS. The resulting mixture was concentrated under reduced pressure to afford tert-butyl rel-methyl((6-((4bR,8aR)-4b,7,7-trimethyl-2-(methylsulfinyl)-4b,7,8,8a-tetrahydropyrano[3′,4′:4,5]pyrrolo[2,3-d]pyrimidin-9(5H)-yl)pyridin-2-yl)methyl)carbamate (60 mg, crude) as a yellow solid. LCMS (ES, m/z): 502 [M+H]⁺. Rt 0.721 min.

### Step 6. tert-butyl methyl((6-((4bR,8aR)-4b,7,7-trimethyl-2-((4-(piperidin-1-yl)phenyl)amino)-4b,7,8,8a-tetrahydropyrano[3′,4′:4,5]pyrrolo[2,3-d]pyrimidin-9(5H)-yl)pyridin-2-yl)methyl)carbamate

### And tert-butyl methyl((6-((4bS,8aS)-4b,7,7-trimethyl-2-((4-(piperidin-1-yl)phenyl)amino)-4b,7,8,8a-tetrahydropyrano[3′,4′:4,5]pyrrolo[2,3-d]pyrimidin-9(5H)-yl)pyridin-2-yl)methyl)carbamate

A mixture of tert-butyl rel-methyl((6-((4bR,8aR)-4b,7,7-trimethyl-2-(methylsulfinyl)-4b,7,8,8a-tetrahydropyrano[3′,4′:4,5]pyrrolo[2,3-d]pyrimidin-9(5H)-yl)pyridin-2-yl)methyl)carbamate (40 mg, 79.74 umol, 1 eq.), Intermediate 4 (72 mg, 0.41 mmol, 5 eq.) and DIPEA (0.2 mL) in toluene (0.5 mL) was stirred at 110° C. for 4 h. The reaction was monitored by LC-MS. The mixture was allowed to cool down to room temperature. The crude product was concentrated under reduced pressure and purified by Prep-HPLC with the following conditions (Kinetex EVO C18 Column, 30×150, 5 um; Mobile Phase A: water (10 mmol/L NH₄HCO₃+0.1% NH₃—H₂O), Mobile Phase B: ACN (70% to 90% in 7 min); 254 nm; Flow rate: 60 mL/min; Rt: 6.35) to afford the racemic product. The mixture was separated by Prep-Chiral-HPLC (Column: CHIRALPAK AD-H, 2×25 cm, 5 μm; Mobile Phase A: Hex (0.5% 2M NH₃-MeOH)-HPLC, Mobile Phase B: EtOH-HPLC (10% to 10% in 38 min); Flow rate: 40 mL/min; Wave Length: 220/254 nm; RT1(min): 27.6; RT2(min): 34) to afford tert-butyl methyl((6-((4bR,8aR)-4b,7,7-trimethyl-2-((4-(piperidin-1-yl)phenyl)amino)-4b,7,8,8a-tetrahydropyrano[3′,4′:4,5]pyrrolo[2,3-d]pyrimidin-9(5H)-yl)pyridin-2-yl)methyl)carbamate (11 mg, 23% yield) as a yellow oil and tert-butyl methyl((6-((4bS,8aS)-4b,7,7-trimethyl-2-((4-(piperidin-1-yl)phenyl)amino)-4b,7,8,8a-tetrahydropyrano[3′,4′:4,5]pyrrolo[2,3-d]pyrimidin-9(5H)-yl)pyridin-2-yl)methyl)carbamate (16 mg, 32% yield) as a yellow oil. LCMS (ES, m/z): 614 [M+H]⁺. Rt 0.917 min.

### Step 7. Example 1

To a stirred solution of tert-butyl methyl((6-((4bS,8aS)-4b,7,7-trimethyl-2-((4-(piperidin-1-yl)phenyl)amino)-4b,7,8,8a-tetrahydropyrano[3′,4′:4,5]pyrrolo[2,3-d]pyrimidin-9(5H)-yl)pyridin-2-yl)methyl)carbamate (11 mg, 17.60 umol, 1 eq.) in DCM (1 mL) was added TFA (1 mL) dropwise. The resulting mixture was stirred at r.t. for 1 h. The reaction was monitored by LC-MS. The resulting mixture was concentrated under reduced pressure. The crude product was purified by Prep-HPLC with the following conditions (Column: XBridge Prep OBD C18 Column, 30×150 mm, 5 um; Mobile Phase A: water (50 mmol/L NH₄HCO₃), Mobile Phase B: ACN (64% to 86% in 7 min); Flow rate:60 mL/min; 254 nm; RT1: 5.27) to afford Example 1 (3.6 mg, 39% yield) as a white solid. ¹HNMR (400 MHz, DMSO-d6) δ 9.06 (s, 1H), 8.49 (d, J=8.4 Hz, 1H), 7.98 (s, 1H), 7.74 (t, J=8.0 Hz, 1H), 7.53 (d, J=9.2 Hz, 2H), 7.04 (d, J=7.6 Hz, 1H), 6.89 (d, J=9.2 Hz, 2H), 4.69-4.66 (m, 1H), 3.75-3.64 (m, 4H), 3.06-3.03 (m, 4H), 2.34 (s, 3H), 2.22-2.17 (m, 1H), 1.65-1.61 (m, 4H), 1.52-1.51 (m, 2H), 1.28-1.23 (m, 7H), 1.03 (s, 3H). LCMS (ES, m/z): 514 [M+H]⁺. Rt 0.946 min.

### Step 8. Example 2

To a stirred solution of tert-butyl methyl((6-((4bR,8aR)-4b,7,7-trimethyl-2-((4-(piperidin-1-yl)phenyl)amino)-4b,7,8,8a-tetrahydropyrano[3′,4′:4,5]pyrrolo[2,3-d]pyrimidin-9(5H)-yl)pyridin-2-yl)methyl)carbamate (16 mg, 25.25 umol, 1 eq.) in DCM (1 mL) was added TFA (1 mL) dropwise. The resulting mixture was stirred at r.t. for 1 h. The crude product was purified by Prep-HPLC with the following conditions (Column: XBridge Prep OBD C18 Column, 30×150 mm, 5 um; Mobile Phase A: water (50 mmol/L NH₄HCO₃), Mobile Phase B: ACN (66% to 86% in 7 min); Flow rate:25 mL/min; 254 nm; RT1: 5.27) to afford Example 2 (4.1 mg, 31% yield) as a white solid. ¹H NMR (400 MHz, DMSO-d6) δ 9.06 (s, 1H), 8.49 (d, J=8.4, 1H), 7.98 (s, 1H), 7.74 (t, J=8.0 Hz, 1H), 7.53 (d, J=8.8 Hz, 2H), 7.04 (d, J=7.2, 1H), 6.89 (d, J=8.8 Hz, 2H), 4.69-4.66 (m, 1H), 3.75-3.66 (m, 4H), 3.06-3.04 (m, 4H), 2.34 (s, 3H), 2.22-2.17 (m, 1H), 1.65-1.63 (m, 4H), 1.52-1.51 (m, 2H), 1.27-1.22 (m, 7H), 1.01 (s, 3H). LCMS (ES, m/z): 514 [M+H]⁺. Rt 0.946 min.

### Examples 3 and 4: 2-(6-((4bS,8aS)-4b,7,7-trimethyl-2-((4-(4-methylpiperazin-1-yl)phenyl) amino)-4b,7,8,8a-tetrahydropyrano[3′,4′:4,5]pyrrolo[2,3-d]pyrimidin-9(5H)-yl)pyridin-2-yl)propan-2-ol and 2-(6-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)pyridin-2-yl)propan-2-ol

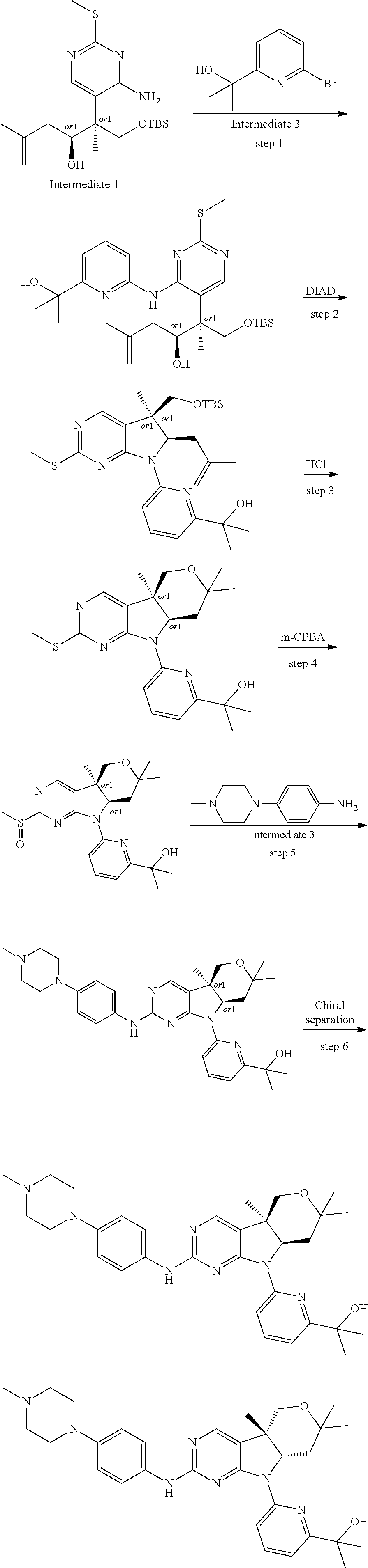

### Step 1. rel-(2S,3S)-1-((tert-butyldimethylsilyl)oxy)-2-(4-((6-(2-hydroxypropan-2-yl)pyridin-2-yl)amino)-2-(methylthio)pyrimidin-5-yl)-2,5-dimethylhex-5-en-3-ol

To a stirred mixture of rel-(2S,3S)-2-(4-amino-2-(methylthio)pyrimidin-5-yl)-1-((tert-butyldimethylsilyl)oxy)-2,5-dimethylhex-5-en-3-ol (Intermediate 1, 90 mg, 0.23 mmol, 1 eq.), Intermediate 3 (54 mg, 0.25 mmol, 1.1 eq.), CuI (43 mg, 0.23 mmol, 1 eq.) and K₂CO₃ (63 mg, 0.45 mmol, 2 eq.) in 1,4-dioxane (3 mL) was added DMEDA (20 mg, 0.23 mmol, 1 eq.) at 60° C. under N₂ atmosphere. The resulting mixture was stirred at 100° C. for 16 h under N₂ atmosphere. The reaction was monitored by LC-MS. The mixture was allowed to cool down to room temperature. The resulting mixture was concentrated under reduced pressure. The residue was purified by silica gel column chromatography (eluting with 1:1 EA/PE) to afford rel-(2S,3S)-1-((tert-butyldimethylsilyl)oxy)-2-(4-((6-(2-hydroxypropan-2-yl)pyridin-2-yl)amino)-2-(methylthio)pyrimidin-5-yl)-2,5-dimethylhex-5-en-3-ol (92 mg, 76% yield) as a yellow solid. LCMS (ES, m/z): 533 [M+H]⁺, Rt 1.082 min.

### Step 2. rel-2-(6-((5R,6R)-5-(((tert-butyldimethylsilyl)oxy)methyl)-5-methyl-6-(2-methylallyl)-2-(methylthio)-5,6-dihydro-7H-pyrrolo[2,3-d]pyrimidin-7-yl)pyridin-2-yl)propan-2-ol

To a stirred solution of rel-(2S,3S)-1-((tert-butyldimethylsilyl)oxy)-2-(4-((6-(2-hydroxypropan-2-yl)pyridin-2-yl)amino)-2-(methylthio)pyrimidin-5-yl)-2,5-dimethylhex-5-en-3-ol (92 mg, 0.17 mmol, 1 eq.) and PPh₃ (91 mg, 0.35 mmol, 2 eq.) in THF (4 mL) was added DIAD (70 mg, 0.35 mmol, 2 eq.) at r.t. under N₂ atmosphere. The resulting mixture was stirred for 3 h at 50° C. under N₂ atmosphere. The reaction was monitored by TLC. The mixture was allowed to cool down to room temperature. The resulting mixture was quenched with ice/water (10 mL) and was extracted with EA (2×10 ml). The combined organic layers were washed with brine (2×20 ml), dried over anhydrous Na₂SO₄. After filtration, the filtrate was concentrated under reduced pressure. The residue was purified by silica gel column chromatography (eluting with 1:2 EA/PE) to afford rel-2-(6-((5R,6R)-5-(((tert-butyldimethylsilyl)oxy)methyl)-5-methyl-6-(2-methylallyl)-2-(methylthio)-5,6-dihydro-7H-pyrrolo[2,3-d]pyrimidin-7-yl)pyridin-2-yl)propan-2-ol (70 mg, 79% yield) as a yellow solid. LCMS (ES, m/z): 515 [M+H]⁺, Rt 1.187 min.

### Step 3. rel-2-(6-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)propan-2-ol

To a stirred solution of rel-2-(6-((5R,6R)-5-(((tert-butyldimethylsilyl)oxy)methyl)-5-methyl-6-(2-methylallyl)-2-(methylthio)-5,6-dihydro-7H-pyrrolo[2,3-d]pyrimidin-7-yl)pyridin-2-yl)propan-2-ol (70 mg, 0.14 mmol, 1 eq.) in 1,4-dioxane (0.5 mL) was added HCl (4 M in 1,4-dioxane, 2 mL) at 0° C. The resulting mixture was stirred at r.t. for 3 h. The reaction was monitored by LCMS. The resulting mixture was quenched with water (10 mL). The pH value of the mixture was adjusted to 7-8 with NaOH (3 M in water). The resulting mixture was extracted with EA (2×10 ml). The combined organic layers were washed with brine (2×15 ml), dried over anhydrous Na₂SO₄. After filtration, the filtrate was concentrated under reduced pressure. The residue was purified by silica gel column chromatography (eluting with 1:1 EA/PE) to afford rel-2-(6-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)propan-2-ol (40 mg, 73% yield) as a yellow solid. LCMS (ES, m/z): 401 [M+H]⁺, Rt 0.828 min.

### Step 4. rel-2-(6-((4bR,8aR)-4b,7,7-trimethyl-2-(methylsulfinyl)-4b,7,8,8a-tetrahydropyrano[3′,4′:4,5]pyrrolo[2,3-d]pyrimidin-9(5H)-yl)pyridin-2-yl)propan-2-ol

To a stirred solution of rel-2-(6-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)propan-2-ol (40 mg, 99.87 umol, 1 eq.) in toluene (1 mL) was added m-CPBA (22 mg, 0.13 mmol, 1.3 eq.) at 0° C. The resulting mixture was stirred for 0.5 h at r.t. The reaction was monitored by TLC. The resulting mixture was concentrated under reduced pressure to afford rel-2-(6-((4bR,8aR)-4b,7,7-trimethyl-2-(methylsulfinyl)-4b,7,8,8a-tetrahydropyrano[3′,4′:4,5]pyrrolo[2,3-d]pyrimidin-9(5H)-yl)pyridin-2-yl)propan-2-ol (55 mg, crude) as a yellow oil. LCMS (ES, m/z):417 [M+H]⁺, Rt 0.753 min.

### Step 5. rel-2-(6-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)pyridin-2-yl)propan-2-ol

To a stirred solution of rel-2-(6-((4bR,8aR)-4b,7,7-trimethyl-2-(methylsulfinyl)-4b,7,8,8a-tetrahydropyrano[3′,4′:4,5]pyrrolo[2,3-d]pyrimidin-9(5H)-yl)pyridin-2-yl)propan-2-ol (55 mg, 0.13 mmol, 1 eq.) and DIPEA (0.5 mL) in toluene (0.5 mL) was added Intermediate 5 (76 mg, 396.12 umol, 3 eq.). The resulting mixture was stirred at 100° C. for 16 h. The reaction was monitored by LCMS. The mixture was allowed to cool down to room temperature and was concentrated under reduced pressure. The crude product was purified by Prep-HPLC (C18; mobile phase: A, water (containing 10 mM NH₄HCO₃) and B, ACN (5% to 80% over 35 min) to afford rel-2-(6-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)pyridin-2-yl)propan-2-ol (25 mg, 71% yield) as a yellow solid. LCMS (ES, m/z): 544 [M+H]⁺, Rt 0.563 min.

### Step 6: Separation of enantiomers to obtain 2-(6-((4bS,8aS)-4b,7,7-trimethyl-2-((4-(4-methylpiperazin-1-yl)phenyl) amino)-4b,7,8,8a tetrahydropyrano[3′,4′:4,5]pyrrolo[2,3-d]pyrimidin-9(5H)-yl)pyridin-2-yl) propan-2-ol and 2-(6-((4bR,8aR)-4b,7,7-trimethyl-2-((4-(4-methylpiperazin-1-yl)phenyl) amino)-4b,7,8,8a-tetrahydropyrano[3′,4′:4,5]pyrrolo[2,3-d]pyrimidin-9(5H)-yl)pyridin-2-yl)propan-2-ol (Examples 3 and 4)

The racemic mixture containing rel-2-(6-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)pyridin-2-yl)propan-2-ol (25 mg) was separated by Chiral-Prep-HPLC: (CHIRAL ART Cellulose-SC, 2×25 cm, 5 um; Mobile Phase A: Hex (0.5% 2M NH₃-MeOH) Mobile Phase B: EtOH-HPLC; Flow rate:20 mL/min; Gradient: 25% B to 25% B in 13 min; 254/220 nm; RT1:9.875; RT2:11.455). The first eluting isomer (RT 9.875 min) was concentrated to afford Example 3 (8.4 mg) as a white solid. The second eluting isomer (RT 11.455 min) was concentrated to afford Example 4 (8.3 mg) as a white solid.

Example 3: ¹HNMR (300 MHz, DMSO-d6) δ 9.06 (s, 1H), 8.47 (d, J=8.4 Hz, 1H), 7.95 (s, 1H), 7.75 (t, J=8.1 Hz, 1H), 7.54 (d, J=9.0 Hz, 2H), 7.27 (d, J=7.5 Hz, 1H), 6.88 (d, J=9.0 Hz, 2H), 5.14 (s, 1H), 4.63-4.58 (m, 1H), 3.78-3.63 (m, 2H), 3.09-3.03 (m, 4H), 2.45-2.43 (m, 4H), 2.28-2.22 (m, 4H), 1.58-1.52 (m, 1H), 1.45 (s, 6H), 1.28 (s, 3H), 1.23 (s, 3H), 1.03 (s, 3H). LCMS (ES, m/z): 544 [M+H]⁺. Rt 0.628 min.

Example 4: ¹HNMR (300 MHz, DMSO-d6) δ 9.06 (s, 1H), 8.47 (d, J=8.4 Hz, 1H), 7.95 (s, 1H), 7.75 (t, J=7.8 Hz, 1H), 7.54 (d, J=9.0 Hz, 2H), 7.27 (d, J=7.5 Hz, 1H), 6.88 (d, J=9.0 Hz, 2H), 5.14 (s, 1H), 4.64-4.58 (m, 1H), 3.78-3.63 (m, 2H), 3.09-3.05 (m, 4H), 2.49-2.46 (m, 4H), 2.27-2.20 (m, 4H), 1.62-1.53 (m, 1H), 1.45 (s, 6H), 1.28 (s, 3H), 1.23 (s, 3H), 1.03 (s, 3H). LCMS (ES, m/z): 544 [M+H]⁺. Rt 0.627 min.

TABLE 13
The examples in the following table were synthesized according to the
appropriate method as demonstrated in Examples 1-4. Absolute stereochemistry of the
examples in the following table was arbitrarily assigned.

| Structure | Ex # | ¹H NMR | LCMS |
|---|---|---|---|
| 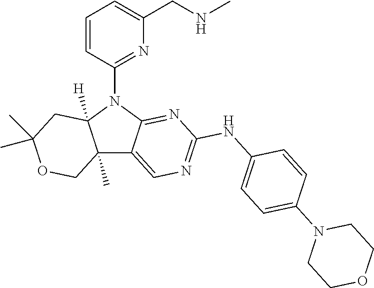 [SMILES: CNCc1cccc(N2c3nc(Nc4ccc(N5CCOCC5)cc4)ncc3[C@@]3(C)COC(C)(C)C[C@@H]23)n1] | 5, 6 | 1H NMR (300 MHz, DMSO-d6) δ 9.11 (s, 1H), 8.48 (d, J = 8.4 Hz, 1H), 7.98 (s, 1H), 7.77-7.72 (m, 1H), 7.57 (d, J = 8.4 Hz, 2H), 7.03 (d, J = 7.5 Hz, 1H), 6.90 (d, J = 8.4 Hz, 2H), 4.70-4.65 (m, 1H), 3.78- 3.71 (m, 7H), 3.66-3.62 (m, 1H), 3.06- 2.93 (m, 4H), 2.34 (s, 3H), 2.21-2.16 (m, 1H), 1.66-1.58 (m, 1H), 1.27 (s, 3H), 1.22 (s, 3H), 1.00 (s, 3H). 1H NMR (300 MHz, DMSO-d6) δ 9.11 (s, 1H), 8.49 (d, J = 8.4 Hz, 1H), 7.99 (s, 1H), 7.77-7.72 (m, 1H), 7.57 (d, J = 8.4 Hz, 2H), 7.04 (d, J = 7.5 Hz, 1H), 6.91 (d, J = 8.4 Hz, 2H), 4.71-4.65 (m, 1H), 3.79- 3.70 (m, 7H), 3.66-3.62 (m, 1H), 3.05- 3.02 (m, 4H), 2.35 (s, 3H), 2.21-2.17 (m, 1H), 1.66-1.58 (m, 1H), 1.27 (s, 3H), 1.22 (s, 3H), 1.01 (s, 3H). | LCMS (ES, m/z): 516[M + H]+, Rt 0.484 min. LCMS (ES, m/z): 516[M + H]+, Rt 0.483 min. |
| 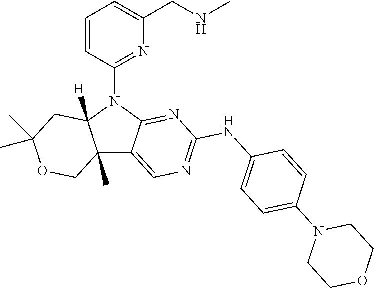 [SMILES: CNCc1cccc(N2c3nc(Nc4ccc(N5CCOCC5)cc4)ncc3[C@]3(C)COC(C)(C)C[C@H]23)n1] | | | |
| 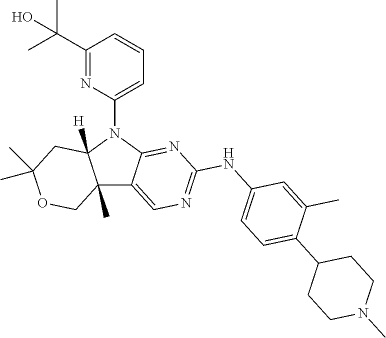 | 7, 8 | 1H NMR (300 MHz, DMSO-d6) δ 9.20 (s, 1H), 8.49 (d, J = 8.4 Hz, 1H), 8.01 (s, 1H), 7.78 (t, J = 7.8 Hz, 1H), 7.58 (s, 1H), 7.43 (d, J = 8.7 Hz, 1H), 7.31 (d, J = 7.5 Hz, 1H), 7.12 (d, J = 7.5 Hz, 1H), 5.17 (s, 1H), 4.67-4.62 (m, 1H), 3.81-3.66 (m, 2H), 2.93-2.90 (m, 2H), 2.74-2.61 (m, 1H), 2.30 (s, 3H), 2.26-2.22 (m, 4H), 2.09-2.00 (m, 2H), 1.75-1.60 (m, 5H), 1.47 (s, 6H), 1.28 (s, 3H), 1.23 (s, 3H), 1.05 (s, 3H). 1H NMR (300 MHz, DMSO-d6) δ 9.20 (s, 1H), 8.49 (d, J = 8.7 Hz, 1H), 8.01 (s, 1H), 7.78 (t, J = 8.1 Hz, 1H), 7.58 (s, 1H), 7.43 (d, J = 7.8 Hz, 1H), 7.31 (d, J = 7.2 Hz, 1H), 7.12 (d, J = 8.4 Hz, 1H), 5.17 (s, 1H), 4.67-4.62 (m, 1H), 3.81-3.66 (m, 2H), 2.93-2.89 (m, 2H), 2.74-2.61 (m, 1H), 2.30 (s, 3H), 2.26-2.23 (m, 4H), 2.08-2.00 | LCMS (ES, m/z): 557 [M + H]+. Rt 0.655 min. LCMS (ES, m/z): 557 [M + H]+. Rt 0.656 min. |
| | | (m, 2H), 1.67-1.60 (m, 5H), 1.47 (s, | |
| | | 6H), 1.31 (s, 3H), 1.25 (s, 3H), 1.05 | |
| | | (s, 3H). | |
| 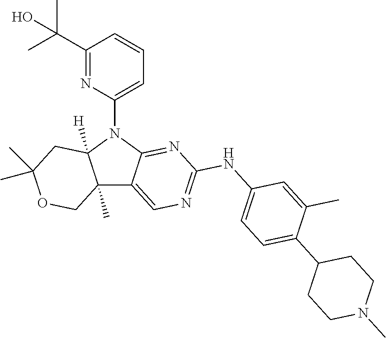 | | | |
| 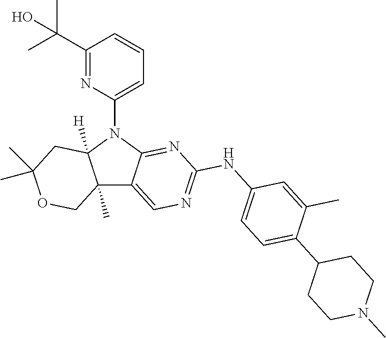 | 9, 10 | 1H NMR (300 MHz, DMSO-d6) δ 9.16 (s, 1H), 8.49 (d, J = 8.4 Hz, 1H), 8.00 (s, 1H), 7.78 (t, J = 7.8 Hz, 1H), 7.61 (s, 1H), 7.41 (d, J = 8.4 Hz, 1H), 7.30 (d, J = 7.5 Hz, 1H), 6.98 (d, J = 8.7 Hz, 1H), 5.17 (s, 1H), 4.67- 4.61 (m, 1H), 3.81-3.66 (m, 2H), 2.85- 2.81 (m, 4H), 2.62-2.52 (m, 4H), 2.30- 2.27 (m, 4H), 2.36 (s, 3H), 1.64-1.56 (m, 1H), 1.47 (s, 6H), 1.31 (s, 3H), 1.25 (s, 3H), 1.05 (s, 3H). 1H NMR (300 MHz, DMSO-d6) δ 9.17 (s, 1H), 8.49 (d, J = 7.5 Hz, 1H), 8.00 (s, 1H), 7.78 (t, J = 7.9 Hz, 1H), 7.61 (s, 1H), 7.42 (d, J = 7.8 Hz, 1H), 7.30 (d, J = 7.5 Hz, 1H), 6.99 (d, J = 7.8 Hz, 1H), 5.18 (s, 1H), 4.69- 4.59 (m, 1H), 3.81-3.66 (m, 2H), 2.92- 2.80 (m, 4H), 2.62-2.54 (m, 4H), 2.36- 2.30 (m, 4H), 2.26 (s, 3H), 1.68-1.58 (m, 1H), 1.59 (s, 6H), 1.31 (s, 3H), 1.25 (s, 3H), 1.05 (s, 3H). | LCMS (ES, m/z): 558 [M + H]+. Rt 2.949 min. LCMS (ES, m/z): 558 [M + H]+. Rt 2.949 min. |
| 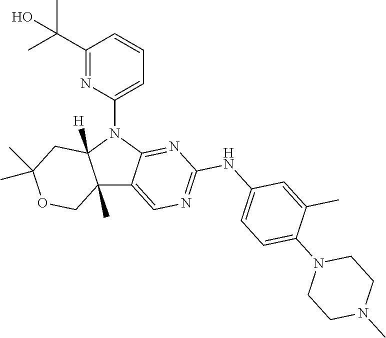 | | | |
| 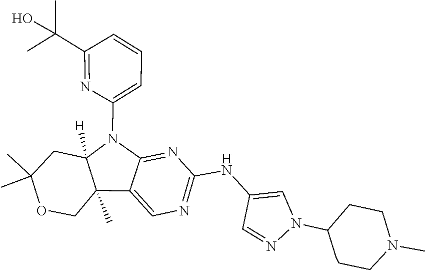 | 11, 12 | 1H NMR (300 MHz, DMSO-d6) δ 9.13 (s, 1H), 8.52-8.37 (m, 1H), 7.95 (s, 1H), 7.89 (s, 1H), 7.75 (t, J = 7.9 Hz, 1H), 7.45 (s, 1H), 7.28 (d, J = 7.2 Hz, 1H), 5.17 (s, 1H), 4.63-4.58 (m, 1H), 4.07-4.03 (m, 1H), 3.78-3.61 (m, 2H), 2.88-2.80 (m, 2H), 2.27-2.17 (m, 4H), 2.09-1.81 (m, 6H), 1.59-1.53 (m, 1H), 1.45 (s, 6H), 1.30 (s, 3H), 1.23 (s, 3H), 1.02 (s, 3H). 1H NMR (300 MHz, DMSO-d6) δ 9.13 (s, 1H), 8.52-8.34 (m, 1H), 7.95 (s, 1H), 7.89 (s, 1H), 7.73 (t, J = 7.9 Hz, 1H), 7.45 (s, 1H), 7.27 (d, J = 7.5 Hz, 1H), 5.17 (s, 1H), 4.62-4.56 (m, 1H), 4.05-3.99 (m, 1H), 3.78-3.60 (m, 2H), 2.88-2.80 (m, 2H), 2.26-2.16 (m, 4H), 2.09-1.83 (m, 6H), 1.59-1.50 (m, 1H), 1.44 (m, 6H), | LCMS (ES, m/z): 533 [M + H]+. Rt 0.614 min. LCMS (ES, m/z): 533 [M + H]+. Rt 0.612 min. |
| | | 1.30 (s, 3H), 1.23 (s, 3H), 1.02 (s, 3H). | |
| 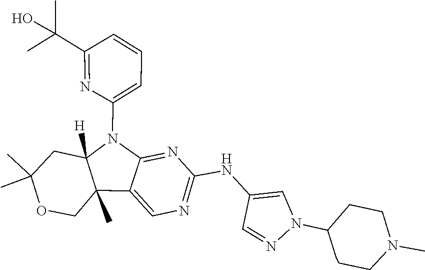 | | | |
| 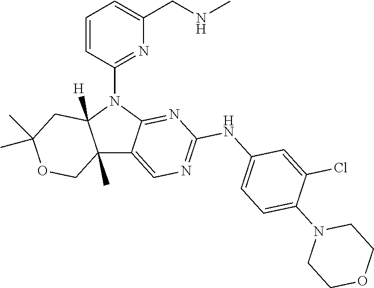 [SMILES: CNCc1cccc(N2c3nc(Nc4ccc(N5CCOCC5)c(Cl)c4)ncc3[C@]3(C)COC(C)(C)C[C@H]23)n1] | 13, 14 | 1H NMR (300 MHz, DMSO-d6) δ 9.44 (s, 1H), 8.45 (d, J = 8.4 Hz, 1H), 8.07-8.05 (m, 2H), 7.79-7.76 (m, 1H), 7.55-7.51 (m, 1H), 7.15-7.07 (m, 2H), 4.74-4.70 (m, 1H), 3.76-3.52 (m, 8H), 2.95-2.87 (m, 4H), 2.29 (s, 3H), 2.24-2.17 (m, 1H), 1.70-1.62 (m, 1H), 1.30 (s, 3H), 1.23 (s, 3H), 1.02 (s, 3H). 1H NMR (300 MHz, DMSO-d6) δ 9.44 (s, 1H), 8.45 (d, J = 8.4 Hz, 1H), 8.07-8.06 (m, 2H), 7.81-7.76 (m, 1H), 7.55-7.51 (m, 1H), 7.15-7.07 (m, 2H), 4.74-4.70 (m, 1H), 3.81-3.52 (m, 8H), 2.96-2.93 (m, 4H), 2.29 (s, 3H), 2.24-2.18 (m, 1H), 1.70-1.62 (m, 1H), 1.30 (s, 3H), 1.23 (s, 3H), 1.02 (s, 3H). | LCMS (ES, m/z): 550 [M+H] +, Rt 0.677 min LCMS (ES, m/z): 550 [M + H]+, Rt 2.854 min |
| 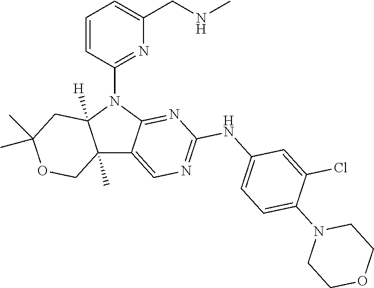 [SMILES: CNCc1cccc(N2c3nc(Nc4ccc(N5CCOCC5)c(Cl)c4)ncc3[C@@]3(C)COC(C)(C)C[C@@H]23)n1] | | | |
| 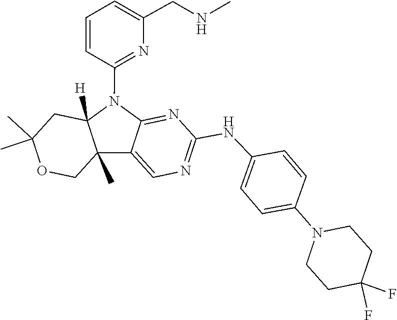 [SMILES: CNCc1cccc(N2c3nc(Nc4ccc(N5CCC(F)(F)CC5)cc4)ncc3[C@]3(C)COC(C)(C)C[C@H]23)n1] | 15, 16 | 1H NMR (300 MHz, DMSO-d6) δ 9.13 (s, 1H), 8.51 (d, J = 8.1 Hz, 1H), 8.01 (s, 1H), 7.77 (t, J = 7.8 Hz, 1H), 7.61 (d, J = 9.0 Hz, 2H), 7.07 (d, J = 7.2 Hz, 1H), 7.00 (d, J = 9.0 Hz, 2H), 4.72-4.67 (m, 1H), 3.80-3.65 (m, 4H), 3.34-3.25 (m, 4H), 2.35 (s, 3H), 2.24-2.03 (m, 5H), 1.68- 1.61 (m, 1H), 1.29 (s, 3H), 1.24 (s, 3H), 1.03 (s, 3H). 1H NMR (300 MHz, DMSO-d6) δ 9.15 (s, 1H), 8.51 (d, J = 8.4 Hz, 1H), 8.00 (s, 1H), 7.77 (t, J = 7.8 Hz, 1H), 7.60 (d, J = 9.0 Hz, 2H), 7.07 (d, J = 7.2 Hz, 1H), 6.99 (d, J = 9.0 Hz, 2H), 4.72-4.67 (m, 1H), 3.79-3.64 (m, 4H), 3.34-3.24 (m, 4H), 2.36 (s, 3H), 2.24-2.02 (m, 5H), 1.68- 1.60 (m, 1H), 1.29 (s, 3H), 1.23 (s, 3H), 1.02 (s, 3H). | LCMS (ES, m/z): 550 [M + H]+. Rt 0.744 min. LCMS (ES, m/z): 550 [M + H]+. Rt 0.740 min. |
| 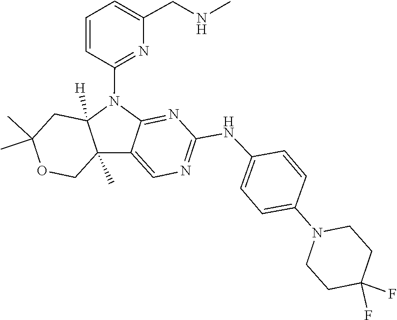 [SMILES: CNCc1cccc(N2c3nc(Nc4ccc(N5CCC(F)(F)CC5)cc4)ncc3[C@@]3(C)COC(C)(C)C[C@@H]23)n1] | | | |
| 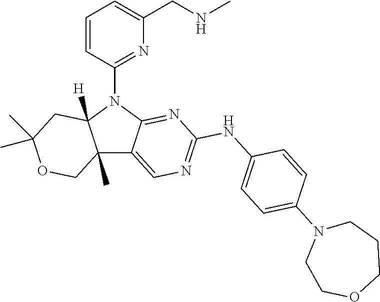 [SMILES: CNCc1cccc(N2c3nc(Nc4ccc(N5CCCOCC5)cc4)ncc3[C@]3(C)COC(C)(C)C[C@H]23)n1] | 17, 18 | 1H NMR (300 MHz, DMSO-d6) δ 8.98 (s, 1H), 8.62 (d, J = 8.7 Hz, 1H), 8.00 (s, 1H), 7.80 (t, J = 7.8 Hz, 1H), 7.47 (d, J = 9.0 Hz, 2H), 7.09 (d, J = 7.2 Hz, 1H), 6.74 (d, J = 9.0 Hz, 2H), 4.81-4.77 (m, 1H), 4.11 (s, 2H), 3.81-3.54 (m, 10H), 2.61 (s, 3H), 2.25-2.17 (m, 1H), 1.96-1.89 (m, 2H), 1.64-1.56 (m, 1H), 1.28 (s, 3H), 1.24 (s, 3H), 1.02 (s, 3H). 1H NMR (300 MHz, DMSO-d6) δ 8.95 (s, 1H), 8.52 (d, J = 8.7 Hz, 1H), 7.97 (s, 1H), 7.72 (t, J = 7.8 Hz, 1H), 7.48 (d, J = 8.7 Hz, 2H), 7.05 (d, J = 7.5 Hz, 1H), 6.74 (d, J = 9.0 Hz, 2H), 4.70-4.66 (m, 1H), 3.79-3.64 (m, 6H), 3.61-3.54 (m, 6H), 2.35 (s, 3H), 2.24-2.18 (m, 1H), 1.97- 1.89 (m, 2H), 1.67-1.59 (m, 1H), 1.28 (s, 3H), 1.23 (s, 3H), 1.03 (s, 3H). | LCMS (ES, m/z): 530 [M + H]+. Rt 0.591 min. LCMS (ES, m/z): 530 [M + H]+. Rt 1.247 min. |
| 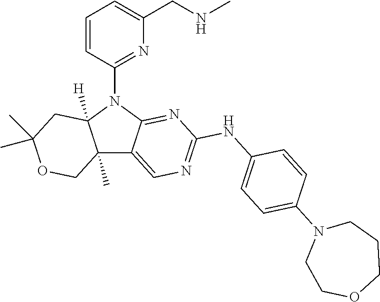 [SMILES: CNCc1cccc(N2c3nc(Nc4ccc(N5CCCOCC5)cc4)ncc3[C@@]3(C)COC(C)(C)C[C@@H]23)n1] | | | |
| 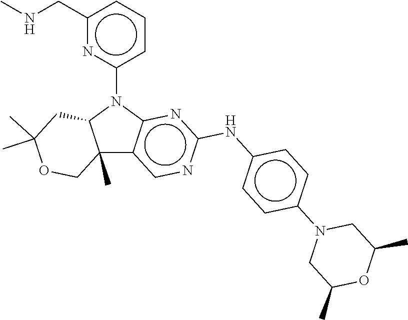 | 19, 20 | 1H NMR (400 MHz, DMSO-d6) δ 9.08 (s, 1H), 8.48 (d, J = 8.4 Hz, 1H), 7.98 (s, 1H), 7.75 (t, J = 8.0 Hz, 1H), 7.56 (d, J = 9.2 Hz, 2H), 7.04 (d, J = 7.2 Hz, 1H), 6.90 (d, J = 8.8 Hz, 2H), 4.69-4.66 (m, 1H), 3.74-3.66 (m, 6H), 3.53-3.45 (m, 2H), 2.34 (s, 3H), 2.23-2.18 (m, 3H), 1.56- 1.63 (m, 1H), 1.27 (s, 3H), 1.22 (s, 3H), 1.16 (d, J = 6.4 Hz, 6H), 1.01 (s, 3H). 1H NMR (400 MHz, DMSO-d6) δ 9.08 (s, 1H), 8.48 (d, J = 8.4 Hz, 1H), 7.98 (s, 1H), 7.75 (t, J = 7.6 Hz, 1H), 7.56 (d, J = 8.8 Hz, 2H), 7.04 (d, J = 7.2 Hz, 1H), 6.90 (d, J = 8.8 Hz, 2H), 4.69-4.66 (m, 1H), 3.74-3.63 (m, 6H), 3.49-3.41 (m, 2H), 2.34 (s, 3H), 2.23-2.18 (m, 3H), 1.65- 1.59 (m, 1H), 1.27 (s, 3H), 1.22 (s, 3H), 1.16 (d, J = 6.4 Hz, 6H), 1.01 (s, 3H). | LCMS (ES, m/z): 544 [M + H]+. Rt 0.631 min. LCMS (ES, m/z): 544 [M + H]+. Rt 0.626 min. |
| 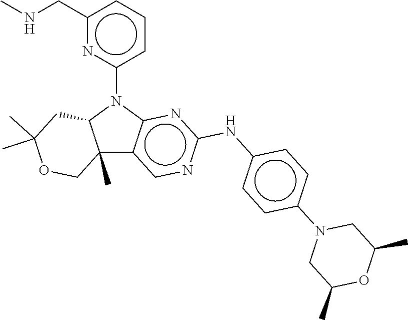 | | | |
| 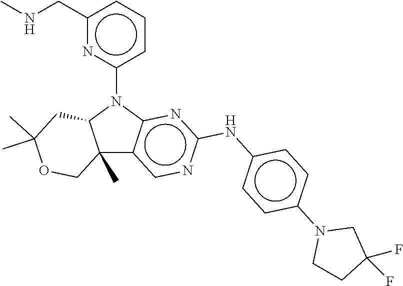 | 21, 22 | 1H NMR (300 MHz, DMSO-d6) δ 9.04 (s, 1H), 8.54 (d, J = 8.4 Hz, 1H), 7.99 (s, 1H), 7.78 (t, J = 7.8 Hz, 1H), 7.57 (d, J = 8.7 Hz, 2H), 7.07 (d, J = 7.2 Hz, 1H), 6.64 (d, J = 9.0 Hz, 2H), 4.73-4.69 (m, 1H), 3.81-3.63 (m, 6H), 3.46 (t, J = 7.2 Hz, 2H), 2.61-2.56 (m, 1H), 2.47-2.42 (m, 4H), 2.24-2.17 (m, 1H), 1.67-1.59 (m, 1H), 1.28 (s, 3H), 1.23 (s, 3H), 1.02 (s, 3H). 1H NMR (300 MHz, DMSO-d6) δ 9.04 (s, 1H), 8.52 (d, J = 8.4 Hz, 1H), 7.99 (s, 1H), 7.77 (t, J = 7.8 Hz, 1H), 7.57 (d, J = 8.7 Hz, 2H), 7.06 (d, J = 7.5 Hz, 1H), 6.64 (d, J = 9.0 Hz, 2H), 4.72-4.68 (m, 1H), 3.79-3.63 (m, 6H), 3.46 (t, J = 7.2 Hz, 2H), 2.61-2.52 (m, 2H), 2.38 (s, 3H), 2.24-2.17 (m, 1H), 1.67-1.59 (m, 1H), | LCMS (ES, m/z): 536 [M + H]+. Rt 0.684 min. LCMS (ES, m/z): 536 [M + H]+. Rt 0.690 min. |
| | | 1.28 (s, 3H), 1.23 (s, 3H), 1.02 (s, 3H). | |
| 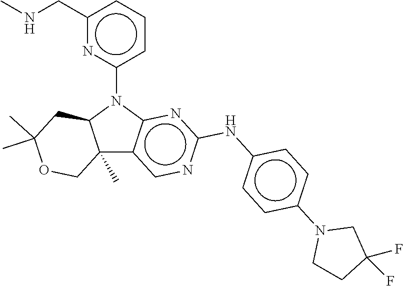 | | | |
| 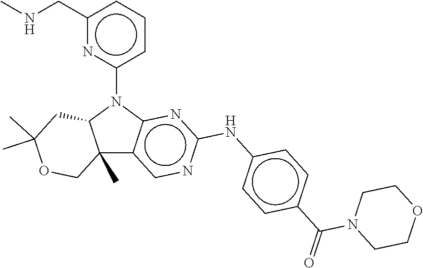 | 23, 24 | 1H NMR (300 MHz, DMSO-d6) δ 9.60 (s, 1H), 8.49 (d, J = 8.1 Hz, 1H), 8.09 (s, 1H), 7.86-7.77 (m, 3H), 7.39 (d, J = 7.8 Hz, 2H), 7.09 (d, J = 7.5 Hz, 1H), 4.74- 4.70 (m, 1H), 3.73-3.68 (m, 4H), 3.68- 3.58 (m, 4H), 3.58-3.52 (m, 4H), 2.36 (s, 3H), 2.24-2.18 (m, 1H), 1.71-1.64 (m, 1H), 1.31 (s, 3H), 1.24 (s, 3H), 1.03 (s, 3H). 1H NMR (300 MHz, DMSO-d6) δ 9.60 (s, 1H), 8.48 (d, J = 8.4 Hz, 1H), 8.09 (s, 1H), 7.88-7.76 (m, 3H), 7.38 (d, J = 8.7 Hz, 2H), 7.08 (d, J = 7.5 Hz, 1H), 4.75- 4.70 (m 1H), 3.78-3.7 (m, 4H), 3.66-3.61 (m, 4H), 3.57-3.49 (m, 4H), 2.35 (s, 3H), 2.24-2.18 (m, 1H), 1.71-1.63 (m, 1H), 1.31 (s, 3H), 1.23 (s, 3H), 1.02 (s, 3H). | LCMS (ES, m/z): 544 [M + H]+, Rt 0.626 min LCMS (ES, m/z): 544 [M + H]+, Rt 0.619 min |
| 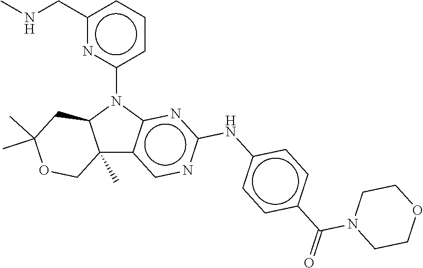 | | | |
| 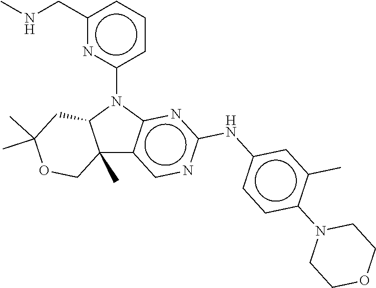 | 25, 26 | 1H NMR (300 MHz, DMSO-d6) δ 9.18 (s, 1H), 8.51 (d, J = 8.7 Hz, 1H), 8.03 (s, 1H), 7.76 (t, J = 7.8 Hz, 1H), 7.64-7.59 (m, 1H), 7.45-7.42 (m, 1H), 7.07 (d, J = 7.2 Hz, 1H), 7.01 (d, J = 8.7 Hz, 1H), 4.73-4.69 (m, 1H), 3.80-3.65 (m, 8H), 2.83-2.80 (m, 4H), 2.37 (s, 3H), 2.28 (s, 3H), 2.24-2.18 (m, 1H), 1.68-1.60 (m, 1H), 1.29 (s, 3H), 1.23 (s, 3H), 1.02 (s, 3H). 1H NMR (300 MHz, DMSO-d6) δ 9.18 (s, 1H), 8.50 (d, J = 8.4 Hz, 1H), 8.02 (s, 1H), 7.75 (t, J = 7.8 Hz, 1H), 7.63-7.59 (m, 1H), 7.45 (d, J = 6.9 Hz, 1H), 7.07 (d, J = 7.5 Hz, 1H), 7.01 (d, J = 8.7 Hz, 1H), 4.72-4.68 (m, 1H), 3.80-3.64 (m, 8H), 2.83-2.80 (m, 4H), 2.35 (s, 3H), 2.28 (s, 3H), 2.24-2.18 (m, 1H), 1.68-1.61 (m, | LCMS (ES, m/z): 530 [M + H]+. Rt 0.683 min. LCMS (ES, m/z): 530 [M + H]+. Rt 0.685 min. |
| | | 1H), 1.29 (s, 3H), 1.23 (s, 3H), 1.02 (s, | |
| | | 3H). | |
| 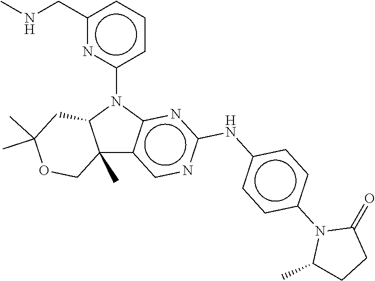 | 27, 28 | 1H NMR (300 MHz, DMSO-d6) δ 9.37 (s, 1H), 8.49 (d, J = 8.4 Hz, 1H), 8.05 (s, 1H), 7.79-7.73 (m, 3H), 7.32 (d, J = 9.0 Hz, 2H), 7.07 (d, J = 7.2 Hz, 1H), 4.73- 4.69 (m, 1H), 4.35-4.30 (m, 1H), 3.81- 3.65 (m, 4H), 2.50-2.39 (m, 1H), 2.36 (s, 3H), 2.33-2.16 (m, 3H), 1.71-1.62 (m, 2H), 1.30 (s, 3H), 1.23 (s, 3H), 1.13 (d, J = 6.3 Hz, 3H), 1.03 (s, 3H). 1H NMR (300 MHz, DMSO-d6) δ 9.37 (s, 1H), 8.50 (d, J = 8.4 Hz, 1H), 8.05 (s, 1H), 7.79-7.73 (m, 3H), 7.20 (d, J = 9 Hz, 2H), 7.07 (d, J = 7.2 Hz, 1H), 4.73-4.69 (m, 1H), 4.35-4.30 (m, 1H), 3.81-3.65 (m, 4H), 2.58-2.54 (m, 1H), 2.36 (s, 3H), 2.33-2.16 (m, 3H), 1.71-1.62 (m, 2H), 1.30 (s, 3H), 1.23 (s, 3H), 1.14 (d, J = 6.3 Hz, 3H), 1.03 (s, 3H). | LCMS (ES, m/z): 528 [M + H]+, Rt 0.644 min LCMS (ES, m/z): 528 [M + H]+, Rt 0.724 min. |
| 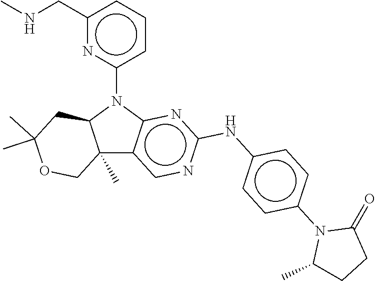 | | | |
| 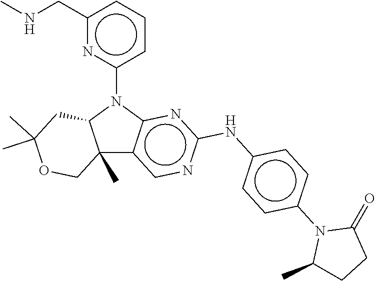 | 29, 30 | 1H NMR (300 MHz, DMSO-d6) δ 9.38 (s, 1H), 8.54 (d, J = 8.1 Hz, 1H), 8.07 (s, 1H), 7.83-7.73 (m, 3H), 7.32 (d, J = 8.7 Hz, 2H), 7.08 (d, J = 7.2 Hz, 1H), 4.78-4.73 (m, 1H), 4.37-4.32 (m, 1H), 3.90 (s, 2H), 3.85-3.68 (m, 2H), 2.47 (s, 3H), 2.41- 2.16 (m, 4H), 1.68-1.64 (m, 2H), 1.30 (s, 3H), 1.24 (s, 3H), 1.14 (d, J = 6.0 Hz, 3H), 1.03 (s, 3H). 1H NMR (300 MHz, DMSO-d6) δ 9.40 (s, 1H), 8.52 (d, J = 8.4 Hz, 1H), 8.07 (s, 1H), 7.82-7.71 (m, 3H), 7.37-7.25 (m, 2H), 7.09 (d, J = 6.6 Hz, 1H), 4.81-4.70 (m, 1H), 4.43-4.26 (m, 1H), 3.81-3.67 (m, 4H), 2.41 (s, 3H), 2.37-2.22 (m, 4H), 1.78-1.58 (m, 2H), 1.31 (s, 3H), 1.25 (s, 3H), 1.15 (d, J = 5.7 Hz, 3H), 1.04 (s, 3H). | LCMS (ES, m/z): 528 [M + H]+, Rt 0.605 min. LCMS (ES, m/z): 528 [M + H]+, Rt 0.608 min |
| 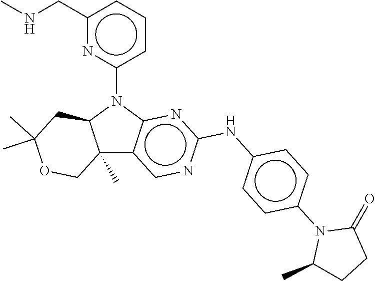 | | | |
| 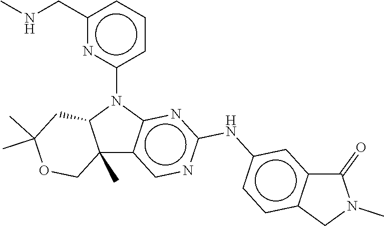 | 31, 32 | 1H NMR (300 MHz, DMSO-d6) δ 9.59 (s, 1H), 8.51 (d, J = 8.4 Hz, 1H), 8.31 (d, J = 1.8 Hz, 1H), 8.10 (s, 1H), 7.90-7.73 (m, 2H), 7.47 (d, J = 8.4 Hz, 1H), 7.08 (d, J = 7.5 Hz, 1H), 4.79-4.70 (m, 1H), 4.41 (s, 2H), 3.83-3.77 (m, 1H), 3.76-3.64 (m, 3H), 3.10 (s, 3H), 2.35 (s, 3H), 2.26-2.16 (m, 1H), 1.73-1.60 (m, 1H), 1.31 (s, 3H), 1.23 (s, 3H), 1.03 (s, 3H). 1H NMR (300 MHz, DMSO-d6) δ 9.59 (s, 1H), 8.51 (d, J = 8.4 Hz, 1H), 8.31 (d, J = 1.8 Hz, 1H), 8.10 (s, 1H), 7.89-7.72 (m, 2H), 7.47 (d, J = 8.1 Hz, 1H), 7.07 (d, J = 7.5 Hz, 1H), 4.78-4.68 (m, 1H), 4.40 | LCMS (ES, m/z): 500[M + H]+, Rt 0.568 min LCMS (ES, m/z): 500[M + H]+, Rt 0.559 min |
| | | (s, 2H), 3.84-3.72 (m, 1H), 3.72-3.62 (m, | |
| | | 3H), 3.10 (s, 3H), 2.35 (s, 3H), 2.27-2.15 | |
| | | (m, 1H), 1.73-1.60 (m, 1H), 1.30 (s, 3H), | |
| | | 1.23 (s, 3H) 1.02 (s, 3H). | |
| 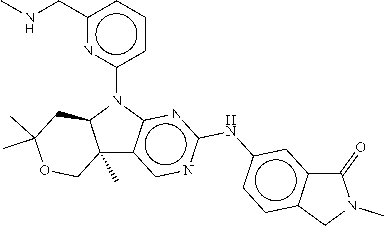 | | | |
| 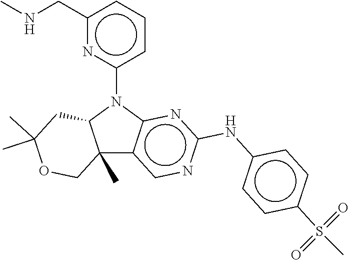 | 33, 34 | 1H NMR (300 MHz, DMSO-d6) δ 9.92 (s, 1H), 8.48 (d, J = 8.4 Hz, 1H), 8.14 (s, 1H), 8.07-7.98 (m, 2H), 7.90-7.77 (m, 3H), 7.11 (d, J = 7.2 Hz, 1H), 4.81-4.71 (m, 1H), 3.84-3.74 (m, 3H), 3.72-3.64 (m, 1H), 3.17 (s, 3H), 2.39 (s, 3H), 2.25-2.15 (m, 1H), 1.75-1.62 (m, 1H), 1.32 (s, 3H), 1.23 (s, 3H), 1.02 (s, 3H). 1H NMR (300 MHz, DMSO-d6) δ 9.91 (s, 1H), 8.47 (d, J = 8.4 Hz, 1H), 8.14 (s, 1H), 8.0-7.98 (m, 2H), 7.88-7.83 (m, 2H), 7.83-7.78 (m, 1H), 7.10 (d, J = 7.5 Hz, 1H), 4.79-4.70 (m, 1H), 3.84-3.76 (m, 1H), 3.75-3.69 (m, 3H), 3.17 (s, 3H), 2.36 (s, 3H), 2.26-2.15 (m, 1H), 1.74- 1.62 (m, 1H), 1.32 (s, 3H), 1.23 (s, 3H), | LCMS (ES, m/z): 509[M + H]+, Rt 0.571 min LCMS (ES, m/z): 509[M + H]+, Rt 0.581 min |
| | | 1.02 (s, 3H). | |
| 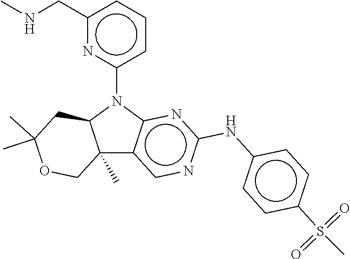 | | | |
| 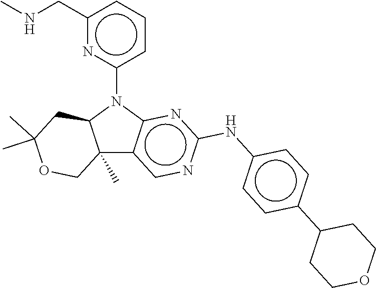 | 35, 36 | 1H NMR (300 MHz, DMSO-d6) δ 9.30 (s, 1H), 8.51 (d, J = 8.4 Hz, 1H), 8.03 (s, 1H), 7.80-7.75 (m, 1H), 7.67 (d, J = 8.4 Hz, 2H), 7.18 (d, J = 8.1 Hz, 2H), 7.07 (d, J = 7.2 Hz, 1H), 4.73-4.46 (m, 1H), 4.02- 3.89 (m, 2H), 3.80-3.65 (m, 4H), 3.49- 3.32 (m, 2H), 2.72 (s, 1H), 2.45 (s, 3H), 2.29-2.18 (m, 1H), 1.69-1.50 (m, 5H), 1.29 (s, 3H), 1.23 (s, 3H), 1.02 (s, 3H). 1H NMR (300 MHz, DMSO-d6) δ 9.26 (s, 1H), 8.50 (d, J = 8.4 Hz, 1H), 8.03 (s, 1H), 7.76 (t, J = 8.1 Hz, 1H), 7.66 (d, J = 8.4 Hz, 2H), 7.17 (d, J = 8.7 Hz, 2H), 7.06 (d, J = 7.5 Hz, 1H), 4.72-4.68 (m, 1H), 3.97- 3.94 (m, 2H), 3.80-3.64 (m, 4H), 3.52-3.31 (m, 2H), 2.76-2.66 (m, 1H), 2.51 (s, 3H), 2.35-2.18 (m, 1H), 1.72-1.61 (m, 5H), 1.29 (s, 3H), 1.23 (s, 3H), 1.05 (s, 3H). | LCMS (ES, m/z): 515 [M+H] +, Rt 0.642 min LCMS (ES, m/z): 515 [M+H] +, Rt 0.648 min |
| 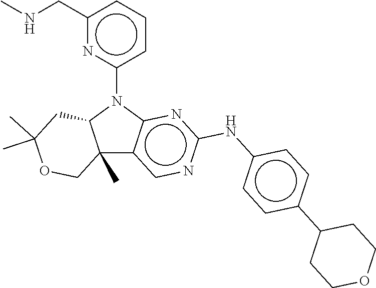 | | | |
| 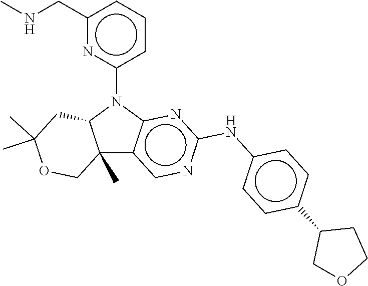 | 37, 38 | 1H NMR (300 MHz, DMSO-d6) δ 9.29 (s, 1H), 8.50 (d, J = 8.1 Hz, 1H), 8.03 (s, 1H), 7.79-7.74 (m, 1H), 7.68 (d, J = 8.4 Hz, 2H), 7.20 (d, J = 8.4 Hz, 2H), 7.06 (d, J = 7.2 Hz, 1H), 4.71-4.68 (m, 1H), 4.06- 3.92 (m, 2H), 3.85-3.69 (m, 2H), 3.65- 3.63 (m, 3H), 3.56-3.51 (m, 1H), 3.36- 3.31 (m, 1H), 2.35-2.18 (m, 5H), 1.96- 1.90 (m, 1H), 1.72-1.57 (m, 1H), 1.29 (s, 3H), 1.23 (s, 3H), 1.02 (s, 3H). 1H NMR (300 MHz, DMSO-d6) δ 9.29 (s, 1H), 8.50 (d, J = 8.1 Hz, 1H), 8.04 (s, 1H), 7.79-7.67 (m, 3H), 7.20 (d, J = 8.7 Hz, 2H), 7.06 (d, J = 7.2 Hz, 1H), 4.71-4.68 (m, 1H), 4.06-3.92 (m, 2H), 3.85-3.72 (m, 2H), 3.69-3.65 (m, 3H), 3.56-3.51 (m, 1H), 3.36-3.32 (m, 1H), 2.35-2.18 | LCMS (ES, m/z): 501 [M + H]+, Rt 0.615 min LCMS (ES, m/z): 501 [M + H]+, Rt 0.612 min |
| | | (m, 5H), 1.97-1.90 (m, 1H), 1.71-1.60 | |
| | | (m, 1H), 1.30 (s, 3H), 1.23 (s, 3H), 1.02 | |
| | | (s, 3H). | |
| 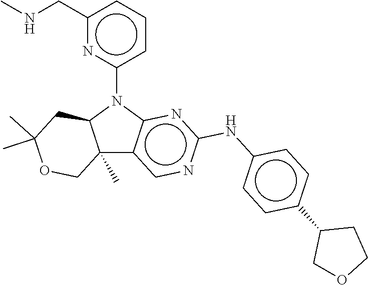 | | | |
| 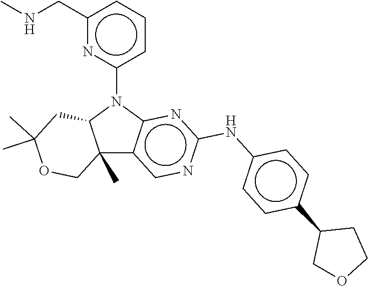 | 39, 40 | 1H NMR (300 MHz, DMSO-d6) δ 9.29 (s, 1H), 8.50 (d, J = 8.4 Hz, 1H), 8.03 (s, 1H), 7.79-7.67 (m, 3H), 7.20 (d, J = 8.7 Hz, 2H), 7.06 (d, J = 7.5 Hz, 1H), 4.73-4.68 (m, 1H), 4.06-3.92 (m, 2H), 3.85-3.65 (m, 5H), 3.56-3.51 (m, 1H), 3.36-3.32 (m, 1H), 2.35-2.18 (m, 5H), 1.97-1.90 (m, 1H), 1.70-1.61 (m, 1H), 1.29 (s, 3H), 1.23 (s, 3H), 1.02 (s, 3H). 1H NMR (300 MHz, DMSO-d6) δ 9.29 (s, 1H), 8.50 (d, J = 8.4 Hz, 1H), 8.03 (s, 1H), 7.79-7.73 (m, 1H), 7.68 (d, J = 8.7 Hz, 2H), 7.20 (d, J = 8.4 Hz, 2H), 7.06 (d, J = 7.5 Hz, 1H), 4.73-4.68 (m, 1H), 4.06- 3.92 (m, 2H), 3.85-3.65 (m, 5H), 3.56- 3.51 (m, 1H), 3.35-3.32 (m, 1H), 2.38- 2.18 (m, 5H), 1.96-1.90 (m, 1H), 1.70- | LCMS (ES, m/z): 501 [M + H]+, Rt 0.624 min LCMS (ES, m/z): 501 [M + H]+, Rt 0.631 min |
| | | 1.60 (m, 1H), 1.29 (s, 3H), 1.23 (s, 3H), | |
| | | 1.02 (s, 3H). | |
| 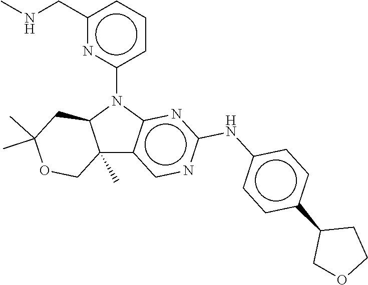 | | | |
| 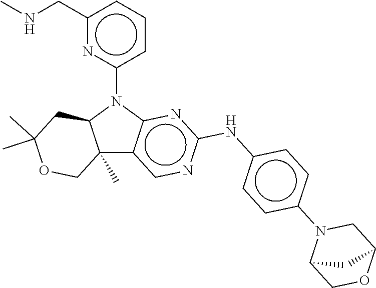 | 41, 42 | 1H NMR (300 MHz, DMSO-d6) δ 8.96 (s, 1H), 8.50 (d, J = 8.1 Hz, 1H), 7.97 (s, 1H), 7.75-7.70 (m, 1H), 7.48 (d, J = 8.7 Hz, 2H), 7.04 (d, J = 7.5 Hz, 1H), 6.60 (d, J = 8.7 Hz, 2H), 4.68-4.51 (m, 3H), 3.79- 3.64 (m, 6H), 3.56-3.51 (m, 1H), 2.97- 2.91 (m, 1H), 2.36 (s, 3H), 2.31-2.16 (m, 1H), 1.98-1.79 (m, 2H), 1.67-1.56 (m, 1H), 1.28 (s, 3H), 1.23 (s, 3H), 1.02 (s, 3H). 1H NMR (300 MHz, DMSO-d6) δ 8.96 (s, 1H), 8.51 (d, J = 8.4 Hz, 1H), 7.97 (s, 1H), 7.76-7.71 (m, 1H), 7.48 (d, J = 8.7 Hz, 2H), 7.04 (d, J = 7.5 Hz, 1H), 6.60 (d, J = 8.7 Hz, 2H), 4.72-4.67 (m, 1H), 4.62- 4.58 (m, 1H), 4.52-4.49 (m, 1H), 3.82- 3.61 (m, 6H), 3.54-3.48 (m, 1H), 2.97- 2.90 (m, 1H), 2.38 (s, 3H), 2.29-2.17 (m, | LCMS (ES, m/z): 528 [M + H]+, Rt 1.117 min LCMS (ES, m/z): 528 [M + H]+, Rt 1.116 min |
| | | 1H), 1.94-1.78 (m, 2H), 1.66-1.59 (m, | |
| | | 1H), 1.28 (s, 3H), 1.23 (s, 3H), 1.02 (s, | |
| | | 3H). | |
| 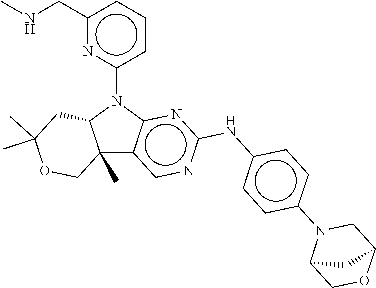 | | | |
| 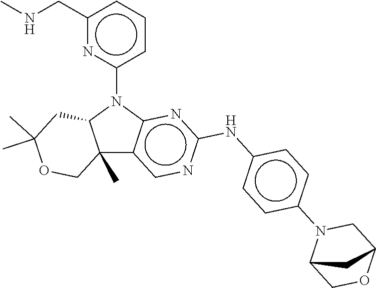 | 43, 44 | 1H NMR (300 MHz, DMSO-d6) δ 8.97 (s, 1H), 8.48 (d, J = 8.1 Hz, 1H), 7.96 (s, 1H), 7.71 (t, J = 7.8 Hz, 1H), 7.48(d, J = 9.0 Hz, 2H), 7.02 (d, J = 7.2 Hz, 1H), 6.59 (d, J = 9.0 Hz, 2H), 4.85-4.42 (m, 3H), 3.80-3.47 (m, 7H), 2.95-2.91 (m, 1H), 2.42-2.12 (m, 4H), 1.99-1.76 (m, 2H), 1.70-1.50 (m, 1H), 1.26 (s, 3H), 1.21 (s, 3H), 1.01 (s, 3H). 1H NMR (300 MHz, DMSO-d6) δ 8.97 (s, 1H), 8.49 (d, J = 8.4 Hz, 1H), 7.96 (s, 1H), 7.71 (t, J = 7.8 Hz, 1H), 7.48 (d, J = 9.0 Hz, 2H), 7.03 (d, J = 7.5 Hz, 1H), 6.60 (d, J = 9.0 Hz, 2H), 4.85-4.42 (m, 3H), 3.80-3.47 (m, 7H), 2.95-2.91 (m, 1H), 2.34 (s, 3H), 2.21-2.15 (m, 1H), 1.96- 1.76 (m, 2H), 1.70-1.50 (m, 1H), 1.26 (s, 3H), 1.21 (s, 3H), 1.01 (s, 3H). | LCMS (ES, m/z): 528 [M + H]+. Rt 0.609 min. LCMS (ES, m/z): 528 [M + H]+. Rt 0.608 min. |
| 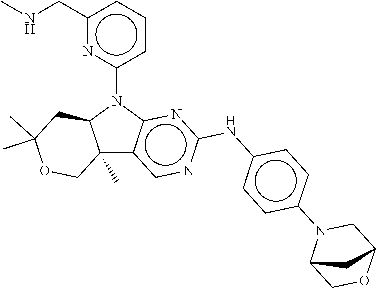 | | | |
| 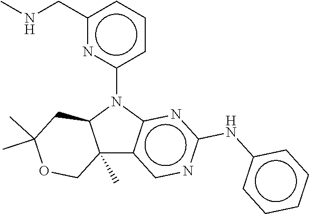 | 45, 46 | 1H NMR (300 MHz, DMSO-d6) δ 9.34 (s, 1H), 8.51 (d, J = 8.4 Hz, 1H), 8.05 (s, 1H), 7.79-7.74 (m, 3H), 7.29 (t, J = 7.8 Hz, 2H), 7.08 (d, J = 7.2 Hz, 1H), 6.94 (t, J = 7.2 Hz, 1H), 4.73-4.69 (m, 1H), 3.81- 3.65 (m, 4H), 2.35 (s, 3H), 2.24-2.18 (m, 1H), 1.69-1.62 (m, 1H), 1.30 (s, 3H), 1.23 (s, 3H), 1.02 (s, 3H). 1H NMR (300 MHz, DMSO-d6) δ 9.34 (s, 1H), 8.51 (d, J = 8.1 Hz, 1H), 8.05 (s, 1H), 7.79-7.74 (m, 3H), 7.29 (t, J = 7.8 Hz, 2H), 7.08 (d, J = 7.2 Hz, 1H), 6.94 (t, J = 7.2 Hz, 1H), 4.73-4.69 (m, 1H), 3.81- | LCMS (ES, m/z): 431 [M + H]+. Rt 0.611 min. LCMS (ES, m/z): 431 [M + H]+. Rt 0.608 min. |
| | | 3.65 (m, 4H), 2.35 (s, 3H), 2.24-2.18 (m, | |
| | | 1H), 1.69-1.62 (m, 1H), 1.30 (s, 3H), | |
| | | 1.23 (s, 3H), 1.02 (s, 3H). | |
| 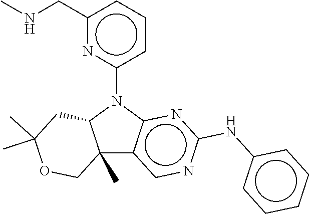 | | | |
| 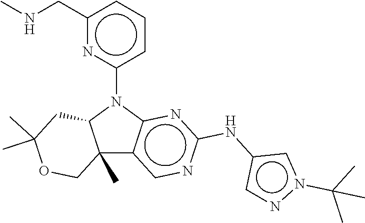 | 47, 48 | 1H NMR (300 MHz, DMSO-d6) δ 9.12 (s, 1H), 8.44-8.38 (m, 1H), 8.00 (s, 1H), 7.98-7.96 (m, 1H), 7.73 (t, J = 7.8 Hz, 1H), 7.48 (s, 1H), 7.07 (d, J = 7.5 Hz, 1H), 4.70-4.65 (m, 1H), 3.78-3.64 (m, 4H), 2.35 (s, 3H), 2.28-2.17 (m, 1H), 1.66-1.58 (m, 1H), 1.52 (s, 9H), 1.28 (s, 3H), 1.23 (s, 3H), 1.02 (s, 3H). 1H NMR (300 MHz, DMSO-d6) δ 9.12 (s, 1H), 8.44-8.38 (m, 1H), 8.00 (s, 1H), 7.98-7.96 (m, 1H), 7.73 (t, J = 7.8 Hz, 1H), 7.48 (s, 1H), 7.07 (d, J = 7.5 Hz, 1H), 4.70-4.65 (m, 1H), 3.78-3.63 (m, 4H), 2.35 (s, 3H), 2.29-2.17 (m, 1H), | LCMS (ES, m/z): 477 [M + H]+. Rt 0.588 min. LCMS (ES) m/z): 477 [M + H]+. Rt 0.586 min. |
| | | 1.66-1.58 (m, 1H), 1.52 (s, 9H), 1.28 (s, | |
| | | 3H), 1.23 (s, 3H), 1.02 (s, 3H). | |
| 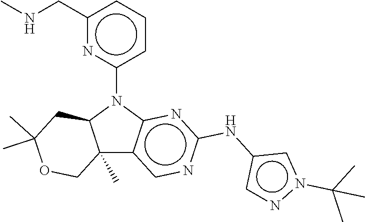 | | | |
| 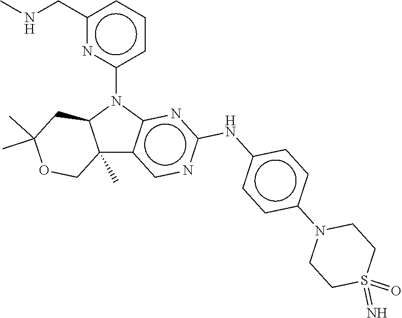 | 49, 50 | 1H NMR (300 MHz, DMSO-d6) δ 9.13 (s, 1H), 8.48 (d, J = 8.4 Hz, 1H), 8.00 (s, 1H), 7.81-7.70 (m, 1H), 7.60 (d, J = 9.0 Hz, 2H), 7.09-6.94 (m, 3H), 4.74-4.64 (m, 1H), 3.82-3.66 (m, 6H), 3.66-3.51 (m, 2H), 3.05-2.99 (m, 4H), 2.35 (s, 3H), 2.24-2.14 (m, 1H), 1.68-1.59 (m, 1H), 1.28 (s, 3H), 1.23 (s, 3H), 1.02 (s, 3H). 1H NMR (300 MHz, DMSO-d6) δ 9.13 (s, 1H), 8.49 (d, J = 8.4 Hz, 1H), 8.00 (s, 1H), 7.81-7.72 (m, 1H), 7.60 (d, J = 9.0 Hz, 2H), 7.05 (d, J = 7.5 Hz, 1H), 6.98 (d, J = 9.0 Hz, 2H), 4.75-4.62 (m, 1H), 3.82- 3.51 (m, 8H), 3.07-2.99 (m, 4H), 2.35 (s, 3H), 2.26-2.15 (m, 1H), 1.70-1.58 (m, 1H), 1.28 (s, 3H), 1.23 (s, 3H), 1.02 (s, 3H). | LCMS (ES, m/z): 563[M + H]+, Rt 0.520 min LCMS (ES, m/z): 563[M + H]+, Rt 0.515 min |
| 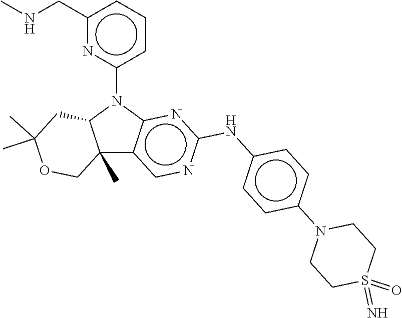 | | | |
| 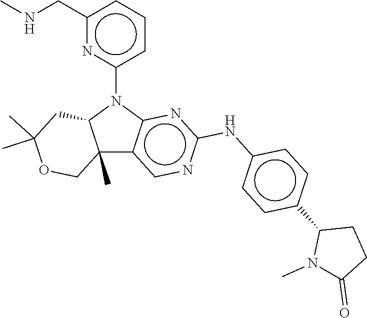 | 51, 52 | 1H NMR (300 MHz, DMSO-d6) δ 9.40 (s, 1H), 8.50 (d, J = 8.4 Hz, 1H), 8.05 (s, 1H), 7.81-7.73 (m, 3H), 7.19 (d, J = 8.1 Hz, 2H), 7.08 (d, J = 7.8 Hz, 1H), 4.73- 4.68 (m, 1H), 4.54-4.47 (m, 1H), 3.81- 3.65 (m, 4H), 2.51-2.35 (m, 9H), 2.24- 2.17 (m, 1H), 1.82-1.61 (m, 2H), 1.30 (s, 3H), 1.23 (s, 3H), 1.02 (s, 3H). 1H NMR (300 MHz, DMSO-d6) δ 9.41 (s, 1H), 8.52 (d, J = 8.7 Hz, 1H), 8.06 (s, 1H), 7.80-7.75 (m, 3H), 7.19 (d, J = 8.4 Hz, 2H), 7.08 (d, J = 7.2 Hz, 1H), 4.74 (m, 1H), 4.52-4.47 (m, 1H), 3.81-3.77 (m, 3H), 3.68-3.62 (m, 1H), 2.51-2.35 (m, 9H), 2.24-2.17 (m, 1H), 1.79-1.75 (m, 1H), 1.69-1.61 (m, 1H), 1.30 (s, 3H), 1.23 (s, 3H), 1.02 (s, 3H). | LCMS (ES, m/z): 528 [M + H]+. Rt 0.569 min LCMS (ES, m/z): 528 [M + H]+. Rt 0.572 min |
| 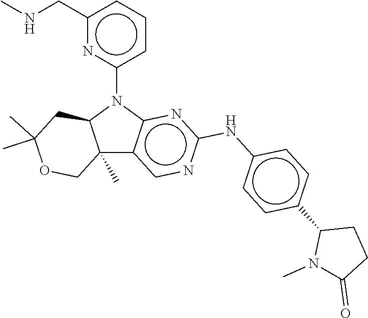 | | | |
| 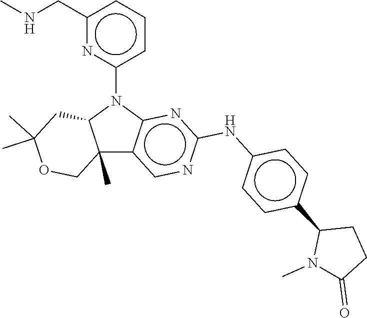 | 53, 54 | 1H NMR (300 MHz, DMSO-d6) δ 9.40 (s, 1H), 8.50 (d, J = 8.4 Hz, 1H), 8.05 (s, 1H), 7.82-7.71 (m, 3H), 7.19 (d, J = 8.1 Hz, 2H), 7.08 (d, J = 7.2 Hz, 1H), 4.73- 4.68 (m, 1H), 4.51-4.48 (m, 1H), 3.81- 3.65 (m, 4H), 2.51-2.35 (m, 9H), 2.22- 2.17 (m, 1H), 1.85-1.75 (m, 1H), 1.70- 1.61 (m, 1H), 1.30 (s, 3H), 1.23 (s, 3H), 1.02 (s, 3H). 1H NMR (300 MHz, DMSO-d6) δ 9.41 (s, 1H), 8.52 (d, J = 8.7 Hz, 1H), 8.06 (s, 1H), 7.80-7.75 (m, 3H), 7.19 (d, J = 8.1 Hz, 2H), 7.09 (d, J = 7.5 Hz, 1H), 4.75- 4.70 (m, 1H), 4.51-4.46 (m, 1H), 3.81- 3.78 (m, 3H), 3.69 (m, 1H), 2.51-2.44 (m, 7H), 2.30-2.17 (m, 3H), 1.80-1.76 (m, 1H), 1.68-1.61 (m, 1H), 1.30 (s, 3H), 1.23 (s, 3H), 1.02 (s, 3H). | LCMS (ES, m/z): 528 [M + H]+. Rt 0.573 min LCMS (ES, m/z): 528 [M + H]+. Rt 0.573 min |
| 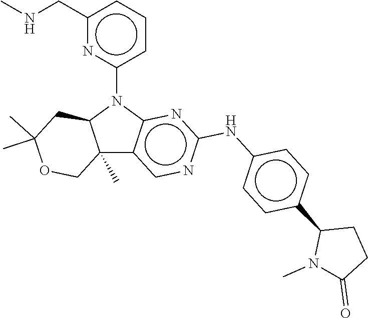 | | | |
| 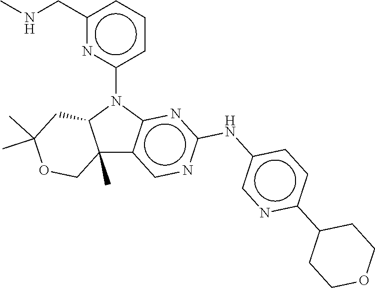 | 55, 56 | 1H NMR (300 MHz, DMSO-d6) δ 9.44 (s, 1H), 8.76 (d, J = 2.7 Hz, 1H), 8.46 (d, J = 8.4 Hz, 1H), 8.17-8.12 (m, 1H), 8.05 (s, 1H), 7.76 (t, J = 7.8 Hz, 1H), 7.22 (d, J = 8.7 Hz, 1H), 7.07 (d, J = 7.5 Hz, 1H), 4.73-4.67 (m, 1H), 4.01-3.90 (m, 2H), 3.82-3.63 (m, 4H), 3.48-3.40 (m, 2H), 2.97-2.78 (m, 1H), 2.34 (s, 3H), 2.23- 2.16 (m, 1H), 1.83-1.59 (m, 5H), 1.30 (s, 3H), 1.23 (s, 3H), 1.01 (s, 3H). 1H NMR (300 MHz, DMSO-d6) δ 9.44 (s, 1H), 8.76 (d, J = 2.7 Hz, 1H), 8.46 (d, J = 8.4 Hz, 1H), 8.16-8.12 (m, 1H), 8.05 (s, 1H), 7.76 (t, J = 7.8 Hz, 1H), 7.22 (d, J = 8.4 Hz, 1H), 7.07 (d, J = 7.5 Hz, 1H), 4.73-4.67 (m, 1H), 4.01-3.90 (m, 2H), 3.82-3.63 (m, 4H), 3.48-3.40 (m, 2H), 2.97-2.81 (m, 1H), 2.34 (s, 3H), 2.23- | LCMS (ES, m/z): 516 [M + H]+. Rt 0.534 min. LCMS (ES, m/z): 516 [M + H]+. Rt 0.536 min. |
| | | 2.16 (m, 1H), 1.82-1.58 (m, 5H), 1.30 (s, | |
| | | 3H), 1.23 (s, 3H), 1.01 (s, 3H). | |
| 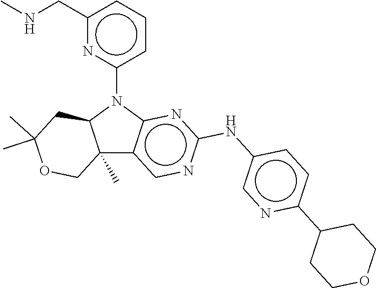 | | | |
| 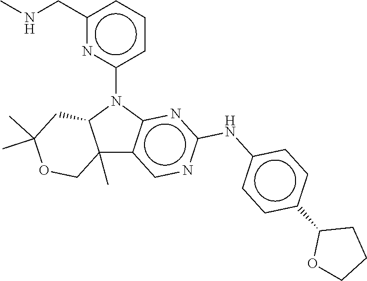 | 57, 59 | 1H NMR (400 MHz, DMSO-d6) δ 9.31 (s, 1H), 8.50 (d, J = 8.4 Hz, 1H), 8.03 (s, 1H), 7.75 (t, J = 7.8 Hz, 1H), 7.70 (d, J = 8.4 Hz, 2H), 7.24 (d, J = 8.4 Hz, 2H), 7.07 (d, J = 7.2 Hz, 1H), 4.75-4.68 (m, 2H), 4.01-3.95 (m, 1H), 3.81-3.65 (m, 5H), 2.35 (s, 3H), 2.29-2.18 (m, 2H), 1.99- 1.91 (m, 2H), 1.74-1.62 (m, 2H), 1.29 (s, 3H), 1.22 (s, 3H), 1.01 (s, 3H). 1H NMR (400 MHz, DMSO-d6) δ 9.31 (s, 1H), 8.50 (d, J = 8.4 Hz, 1H), 8.03 (s, 1H), 7.75 (t, J = 8.0 Hz, 1H), 7.70 (d, J = 8.4 Hz, 2H), 7.24 (d, J = 8.4 Hz, 2H), 7.07 (d, J = 7.2 Hz, 1H), 4.75-4.68 (m, 2H), 4.00-3.95 (m, 1H), 3.81-3.65 (m, 5H), 2.35 (s, 3H), 2.29-2.18 (m, 2H), 1.99- 1.91 (m, 2H), 1.74-1.62 (m, 2H), 1.29 (s, | LCMS (ES, m/z): 501 [M + H]+. Rt 0.544 min. LCMS (ES, m/z): 501 [M + H]+. Rt 0.539 min. |
| | | 3H), 1.22 (s, 3H), 1.01 (s, 3H). | |
| 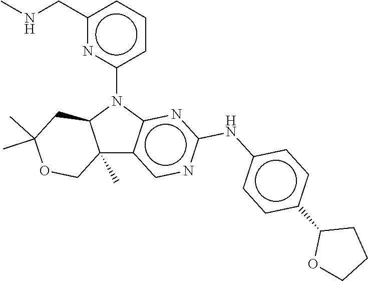 | | | |
| 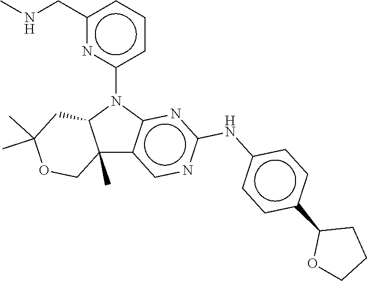 | 58, 60 | 1H NMR (400 MHz, DMSO-d6) δ 9.31 (s, 1H), 8.51 (d, J = 8.4 Hz, 1H), 8.04 (s, 1H), 7.76 (t, J = 8.0 Hz, 1H), 7.70 (d, J = 8.4 Hz, 2H), 7.24 (d, J = 8.4 Hz, 2H), 7.07 (d, J = 7.2 Hz, 1H), 4.75-4.69 (m, 2H), 4.00-3.95 (m, 1H), 3.81-3.75 (m, 4H), 3.68-3.64 (m, 1H), 2.35 (s, 3H), 2.29- 2.17 (m, 2H), 1.99-1.91 (m, 2H), 1.73- 1.61 (m, 2H), 1.29 (s, 3H), 1.22 (s, 3H), 1.01 (s, 3H). 1H NMR (400 MHz, DMSO-d6) δ 9.31 (s, 1H), 8.50 (d, J = 8.0 Hz, 1H), 8.03 (s, 1H), 7.75 (t, J = 8.0 Hz, 1H), 7.70 (d, J = 8.4 Hz, 2H), 7.24 (d, J = 8.8 Hz, 2H), 7.07 (d, J = 7.6 Hz, 1H), 4.75-4.68 (m, 2H), 4.01-3.95 (m, 1H), 3.81-3.65 (m, 5H), 2.35 (s, 3H), 2.29-2.18 (m, 2H), 1.99- | LCMS (ES, m/z): 501 [M + H]+. Rt 0.551 min. LCMS (ES, m/z): 501 [M + H]+. Rt 0.544 min. |
| | | 1.91 (m, 2H), 1.74-1.62 (m, 2H), 1.29 (s, | |
| | | 3H), 1.22 (s, 3H), 1.01 (s, 3H). | |
| 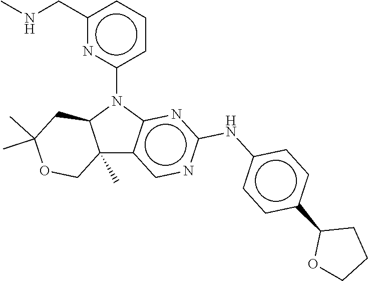 | | | |
| 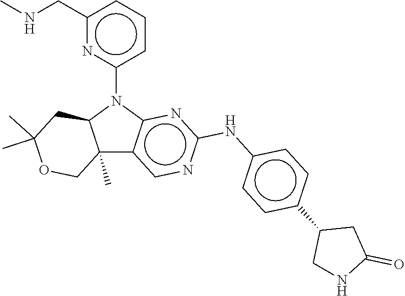 | 61, 62 | 1H NMR (300 MHz, DMSO-d6) δ 9.32 (s, 1H), 8.48 (d, J = 8.4 Hz, 1H), 8.03 (s, 1H), 7.76 (t, J = 7.8 Hz, 1H), 7.68 (d, J = 8.4 Hz, 2H), 7.22 (d, J = 8.7 Hz, 2H), 7.05 (d, J = 7.2 Hz, 1H), 4.80-4.55 (m, 1H), 3.80-3.50 (m, 6H), 3.22-3.08 (m, 1H), 2.48-2.45 (m, 1H), 2.35-2.15 (m, 5H), 1.70-1.55 (m, 1H), 1.27 (s, 3H), 1.22 (s, 3H), 1.01 (s, 3H). 1H NMR (300 MHz, DMSO-d6) δ 9.32 (s, 1H), 8.48 (d, J = 8.4 Hz, 1H), 8.03 (s, 1H), 7.76 (t, J = 7.8 Hz, 1H), 7.68 (d, J = 8.4 Hz, 2H), 7.22 (d, J = 8.7 Hz, 2H), 7.05 (d, J = 7.5 Hz, 1H), 4.80-4.55 (m, 1H), 3.80-3.50 (m, 6H), 3.22-3.08 (m, 1H), 2.40-2.19 (m, 6H), 1.70-1.55 (m, 1H), 1.27 (s, 3H), 1.22 (s, 3H), 1.01 (s, 3H). | LCMS (ES, m/z): 514 [M + H]+. Rt 0.583 min. LCMS (ES, m/z): 514 [M + H]+. Rt 0.576 min. |
| 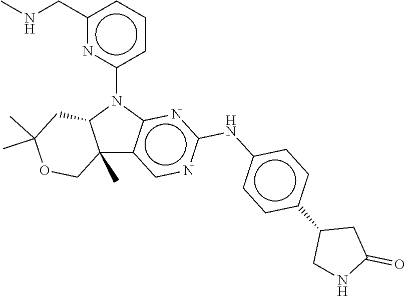 | | | |
| 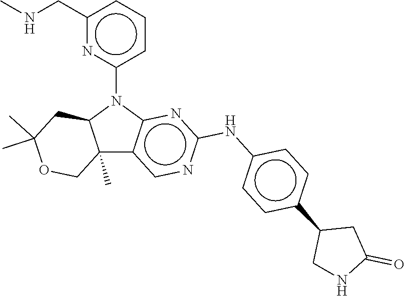 | 63, 64 | 1H NMR (300 MHz, DMSO-d6) δ 9.32 (s, 1H), 8.50 (d, J = 8.4 Hz, 1H), 8.04 (s, 1H), 7.80-7.75 (m, 1H), 7.73-7.65 (m, 3H), 7.23 (d, J = 8.1 Hz, 2H), 7.06 (d, J = 6.9 Hz, 1H), 4.74-4.68 (m, 1H), 3.80-3.64 (m, 4H), 3.61-3.46 (m, 2H), 3.25-3.20 (m, 1H), 2.48-2.40 (m, 1H), 2.30 (s, 3H), 2.28-2.15 (m, 2H), 1.69-1.61 (m, 1H), 1.29 (s, 3H), 1.23 (s, 3H), 1.02 (s, 3H). 1H NMR (300 MHz, DMSO-d6) δ 9.32 (s, 1H), 8.49 (d, J = 8.4 Hz, 1H), 8.04 (s, 1H), 7.80-7.69(m, 4H), 7.24 (d, J = 8.1 Hz, 2H), 7.06 (d, J = 7.2 Hz, 1H), 4.72-4.68 (m, 1H), 3.80-3.69 (m, 4H), 3.64-3.57 (m, 2H), 3.23-3.18 (m, 1H), 2.38-2.35 (m, 1H), 2.33 (s, 3H), 2.32-2.22 (m, 2H), 1.71-1.59 (m, 1H), 1.29 (s, 3H), 1.23 (s, 3H), 1.02 (s, 3H). | LCMS (ES, m/z): 514 [M + H]+, Rt 0.960 min LCMS (ES, m/z): 514 [M + H]+, Rt 0.954 min |
| 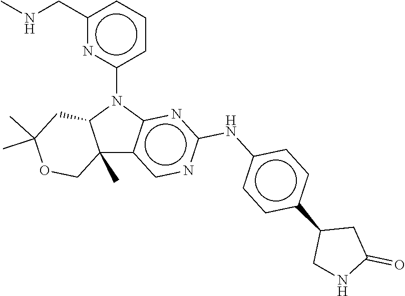 | | | |
| 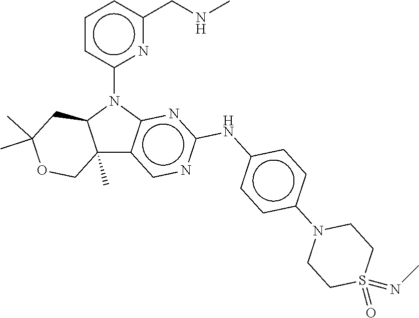 | 65, 66 | 1H NMR (300 MHz, DMSO-d6) δ 9.15 (s, 1H), 8.50 (d, J = 8.4 Hz, 1H), 8.01 (s, 1H), 7.80-7.75 (m, 1H), 7.48 (d, J = 8.4 Hz, 2H), 7.01-6.98 (m, 3H), 4.74-4.69 (m, 1H), 3.82-3.69 (m, 4H), 3.66-3.61 (m, 1H), 3.56-3.49 (m, 2H), 3.19-3.02 (m, 5H), 2.68 (s, 3H), 2.38 (s, 3H), 2.29-2.19 (m, 1H), 1.67-1.60 (m, 1H), 1.28 (s, 3H), 1.23 (s, 3H), 1.02 (s, 3H). 1H NMR (300 MHz, DMSO-d6) δ 9.14 (s, 1H), 8.48 (d, J = 7.5 Hz, 1H), 8.00 (s, 1H), 7.79-7.73 (m, 1H), 7.60 (d, J = 8.7 Hz, 2H), 7.06-6.98 (m, 3H), 4.74-4.69 (m, 1H), 3.79-3.71 (m, 6H), 3.68-3.49 (m, 2H), 3.16-3.06 (m, 2H), 3.06-2.94 (m, 2H), 2.68 (s, 3H), 2.35 (s, 3H), 2.29-2.18 (m, 1H), 1.67-1.60 (m, 1H), 1.28 (s, 3H), 1.23 (s, 3H), 1.02 (s, 3H). | LCMS (ES, m/z): 577 [M + H]+, Rt 0.939 min LCMS (ES, m/z): 577 [M + H]+, Rt 0.932 min |
| 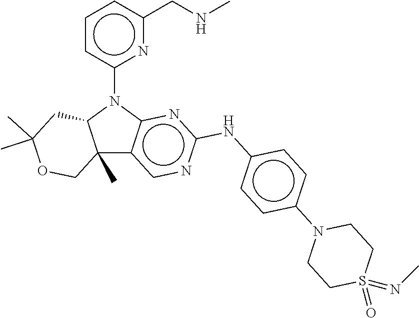 | | | |
| 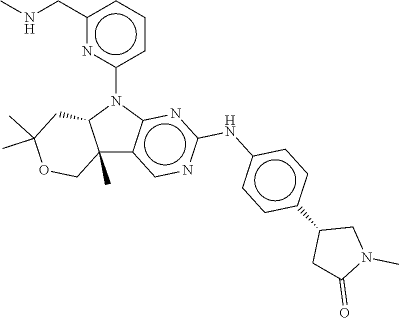 | 67, 68 | 1H NMR (300 MHz, DMSO-d6) δ 9.32 (s, 1H), 8.48 (d, J = 8.4 Hz, 1H), 8.03 (s, 1H), 7.76 (t, J = 7.8 Hz, 1H), 7.69 (d, J = 8.7 Hz, 2H), 7.21 (d, J = 8.7 Hz, 2H), 7.05 (d, J = 7.2 Hz, 1H), 4.80-4.48 (m, 1H), 3.90-3.43 (m, 6H), 3.36-3.34 (m, 1H), 2.77 (s, 3H), 2.70-2.59 (m, 1H), 2.40- 2.28 (m, 4H), 2.25-2.10 (m, 1H), 1.73- 1.45 (m, 1H), 1.27 (s, 3H), 1.22 (s, 3H), 1.01 (s, 3H). 1H NMR (300 MHz, DMSO-d6) δ 9.32 (s, 1H), 8.48 (d, J = 8.4 Hz, 1H), 8.03 (s, 1H), 7.77 (t, J = 7.8 Hz, 1H), 7.69 (d, J = 8.7 Hz, 2H), 7.22 (d, J = 8.7 Hz, 2H), 7.05 (d, J = 7.2 Hz, 1H), 4.79-4.60 (m, 1H), 3.85-3.62 (m, 5H), 3.59-3.45 (m, 1H), 3.36-3.34 (m, 1H), 2.77 (s, 3H), 2.70- 2.58 (m, 1H), 2.43-2.28 (m, 4H), 2.27- 2.15(m, 1H), 1.72-1.55 (m, 1H), 1.27 (s, | LCMS (ES, m/z): 528 [M + H]+. Rt 0.558 min. LCMS (ES, m/z): 528 [M + H]+. Rt 0.575 min. |
| | | 3H), 1.22 (s, 3H), 1.01 (s, 3H). | |
| 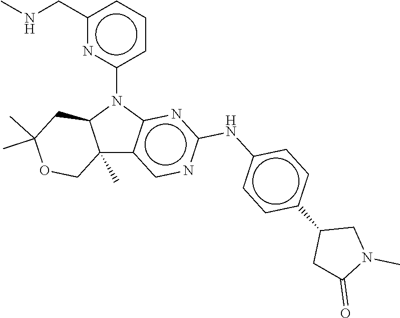 | | | |
| 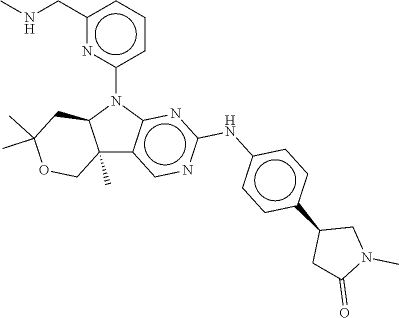 | 69, 70 | 1H NMR (300 MHz, DMSO-d6) δ 9.32 (s, 1H), 8.49 (d, J = 8.4 Hz, 1H), 8.04 (s, 1H), 7.80-7.69 (m, 3H), 7.22 (d, J = 6.0 Hz, 2H), 7.06 (d, J = 7.2 Hz, 1H), 4.74- 4.68 (m, 1H), 3.80-3.64 (m, 5H), 3.57- 3.46 (m, 1H), 3.35-3.32 (m, 1H), 2.83 (s, 3H), 2.67-2.58 (m, 1H), 2.41-2.35 (m, 4H), 2.28-2.18 (m, 1H), 1.71-1.55 (m, 1H), 1.27 (s, 3H), 1.22 (s, 3H), 1.03 (s, 3H). 1H NMR (300 MHz, DMSO-d6) δ 9.32 (s, 1H), 8.48 (d, J = 8.4 Hz, 1H), 8.03 (s, 1H), 7.79-7.68 (m, 3H), 7.22 (d, J = 6.0 Hz, 2H), 7.06 (d, J = 7.2 Hz, 1H), 4.72- 4.68 (m, 1H), 3.80-3.64 (m, 5H), 3.57- 3.46 (m, 1H), 3.40-3.35 (m, 1H), 2.79 (s, 3H), 2.67-2.58 (m, 1H), 2.44-2.38 (m, 4H), 2.23-2.17 (m, 1H), 1.69-1.61 (m, 1H), 1.27 (s, 3H), 1.22 (s, 3H), 1.01 (s, 3H). | LCMS (ES, m/z): 528 [M + H]+. Rt 0.559 min. LCMS (ES, m/z): 528 [M + H]+. Rt 0.560 min. |
| 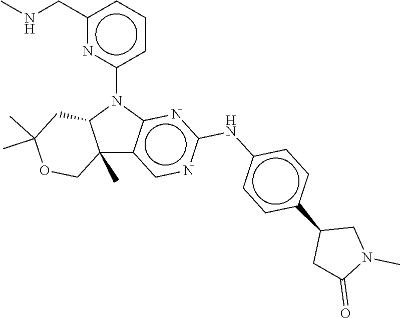 | | | |
| 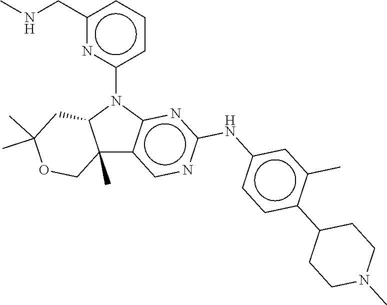 | 71, 72 | 1H NMR (400 MHz, DMSO-d6) δ 9.18 (s, 1H), 8.49 (d, J = 8.4 Hz, 1H), 8.02 (s, 1H), 7.73 (t, J = 8.0 Hz, 1H), 7.56 (s, 1H), 7.43-7.41 (d, J = 7.6 Hz, 1H), 7.12 (d, J = 8.8 Hz, 1H), 7.06 (d, J = 7.2 Hz, 1H), 4.71-4.68 (m, 1H), 3.78-3.64 (m, 4H), 2.90-2.86 (m, 2H), 2.60-2.50 (m, 1H), 2.34 (s, 3H), 2.28 (s, 3H), 2.22-2.18 (m, 4H), 2.01-1.95 (m, 2H), 1.68-1.61 (m, 5H), 1.28 (s, 3H), 1.22 (s, 3H), 1.01 (s, 3H). 1H NMR (400 MHz, DMSO-d6) δ 9.18 (s, 1H), 8.49 (d, J = 8.4 Hz, 1H), 8.02 (s, 1H), 7.73 (t, J = 8.0 Hz, 1H), 7.56 (s, 1H), 7.43-7.41 (d, J = 8.4 Hz, 1H), 7.12 (d, J = 8.4 Hz, 1H), 7.06 (d, J = 8.0 Hz, 1H), 4.71-4.67 (m, 1H), 3.78-3.64 (m, 4H), 2.90-2.86 (m, 2H), 2.62-2.56 (m, 1H), | LCMS (ES, m/z): 542 [M + H]+. Rt 0.665 min. LCMS (ES, m/z): 542 [M + H]+. Rt 0.667 min. |
| | | 2.34 (s, 3H), 2.28 (s, 3H), 2.22-2.18 (m, | |
| | | 4H), 2.01-1.95 (m, 2H), 1.67-1.61 (m, | |
| | | 5H), 1.28 (s, 3H), 1.22 (s, 3H), 1.01 (s, | |
| | | 3H). | |
| 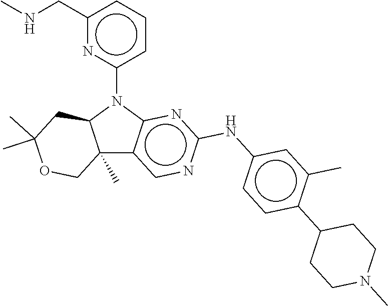 | | | |
| 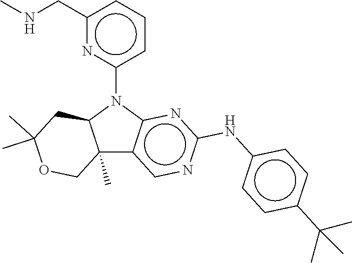 | 73, 74 | 1H NMR (300 MHz, DMSO-d6) δ 9.24 (s, 1H), 8.53 (d, J = 8.4 Hz, 1H), 8.02 (s, 1H), 7.75 (t, J = 7.8 Hz, 1H), 7.66 (d, J = 8.1 Hz, 2H), 7.32 (d, J = 8.1 Hz, 2H), 7.07 (d, J = 7.5 Hz, 1H), 4.72-4.68 (m, 1H), 3.80-3.64 (m, 4H), 2.35 (s, 3H), 2.24- 2.18 (m, 1H), 1.68-1.61 (m, 1H), 1.29 (s, 12H), 1.23 (s, 3H), 1.02 (s, 3H). 1H NMR (300 MHz, DMSO-d6) δ 9.24 (s, 1H), 8.53 (d, J = 8.1 Hz, 1H), 8.02 (s, 1H), 7.75 (t, J = 7.8 Hz, 1H), 7.66 (d, J = 8.1 Hz, 2H), 7.32 (d, J = 8.4 Hz, 2H), 7.07 (d, J = 6.9 Hz, 1H), 4.72-4.68 (m, 1H), 3.80-3.65 (m, 4H), 2.35 (s, 3H), 2.24- 2.18 (m, 1H), 1.68-1.61 (m, 1H), 1.29 (s, 12H), 1.23 (s, 3H), 1.02 (s, 3H). | LCMS (ES, m/z): 487 [M + H]+. Rt 0.735 min. LCMS (ES, m/z): 487 [M + H]+. Rt 0.732 min. |
| 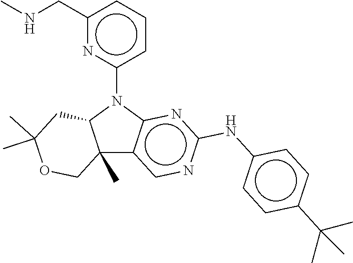 | | | |
| 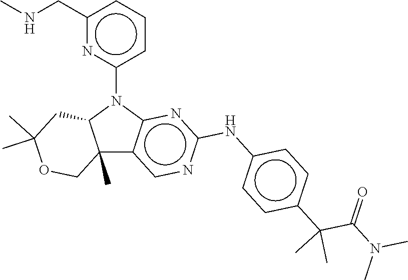 | 75, 76 | 1H NMR (300 MHz, DMSO-d6) δ 9.33 (s, 1H), 8.50 (d, J = 8.4 Hz, 1H), 8.04 (s, 1H), 7.79-7.66 (m, 3H), 7.14-7.04 (m, 3H), 4.75-4.67 (m, 1H), 3.83-3.73 (m, 3H), 3.72-3.63 (m, 1H), 2.92-2.56 (m, 6H), 2.38 (s, 3H), 2.26-2.16 (m, 1H), 1.71- 1.58 (m, 1H), 1.44 (s, 6H), 1.29 (s, 3H), 1.23 (s, 3H), 1.02 (s, 3H). 1H NMR (300 MHz, DMSO-d6) δ 9.32 (s, 1H), 8.49 (d, J = 8.4 Hz, 1H), 8.04 (s, 1H), 7.79-7.66 (m, 3H), 7.14-7.03 (m, 3H), 4.75-4.65 (m, 1H), 3.83-3.63 (m, 4H), 2.92-2.56 (m, 6H), 2.36 (s, 3H), 2.25- 2.15 (m, 1H), 1.72-1.58 (m, 1H), 1.44 (s, 6H), 1.29 (s, 3H), 1.23 (s, 3H), 1.02 (s, 3H). | LCMS (ES, m/z): 544[M + H]+, Rt 0.605 min LCMS (ES, m/z): 544[M + H]+, Rt 0.604 min. |
| 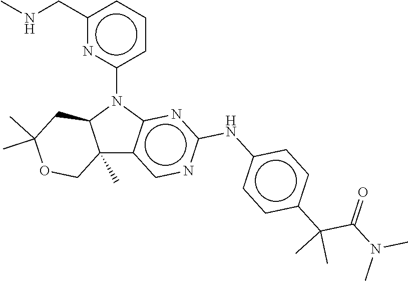 | | | |
| 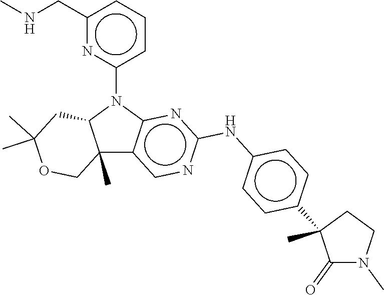 | 77, 78 | 1H NMR (300 MHz, DMSO-d6) δ 9.31 (s, 1H), 8.50 (d, J = 8.1 Hz, 1H), 8.03 (s, 1H), 7.78-7.72 (m, 1H), 7.67 (d, J = 7.8 Hz, 2H), 7.26 (d, J = 8.1 Hz, 2H), 7.06 (d, J = 7.2 Hz, 1H), 4.72-4.68 (m, 1H), 3.84- 3.58 (m, 4H), 2.90 (s, 3H), 2.69-2.63 (m, 2H), 2.39-2.36 (m, 1H), 2.35 (s, 3H), 2.28-2.20 (m, 1H), 2.14-2.09 (m, 1H), 1.70-1.59 (m, 1H), 1.40 (s, 3H), 1.29 (s, 3H), 1.23 (s, 3H), 1.02 (s, 3H). 1H NMR (300 MHz, DMSO-d6) δ 9.30 (s, 1H), 8.50 (d, J = 8.7 Hz, 1H), 8.03 (s, 1H), 7.77-7.72 (m, 1H), 7.67 (d, J = 7.5 Hz, 2H), 7.26 (d, J = 7.5 Hz, 2H), 7.06 (d, J = 6.6 Hz, 1H), 4.72-4.68 (m, 1H), 3.80- 3.65 (m, 4H), 2.81 (s, 3H), 2.74-2.68 (m, 2H), 2.35 (s, 3H), 2.34-2.27 (m, 1H), 2.23-2.18 (m, 1H), 2.13-2.05 (m, 1H), | LCMS (ES, m/z): 542 [M + H]+, Rt 0.599 min LCMS (ES, m/z): 524 [M + H]+, Rt 0.600 min |
| | | 1.68-1.61 (m, 1H), 1.40 (s, 3H), 1.29 (s, | |
| | | 3H), 1.23(s, 3H), 1.02 (s, 3H). | |
| 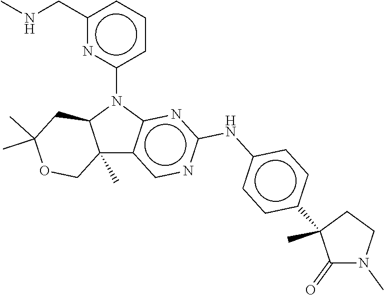 | | | |
| 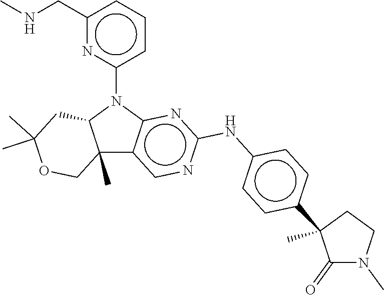 | 79, 80 | 1H NMR (400 MHz, DMSO-d6) δ 9.30 (s, 1H), 8.49 (d, J = 8.0 Hz, 1H), 8.02 (s, 1H), 7.74 (t, J = 8.0 Hz, 1H), 7.66 (d, J = 8.8 Hz, 2H), 7.25 (d, J = 8.4 Hz, 2H), 7.05 (d, J = 7.2 Hz, 1H), 4.70-4.67 (m, 1H), 3.78- 3.75 (m, 1H), 3.71-3.64 (m, 3H), 3.30- 3.24 (m, 2H), 2.80 (s, 3H), 2.38-2.30 (m, 4H), 2.22-2.17 (m, 1H), 2.11-2.08 (m, 1H), 1.69-1.58 (m, 1H), 1.41 (s, 3H), 1.28 (s, 3H), 1.21 (s, 3H), 1.01 (s, 3H). 1H NMR (400 MHz, DMSO-d6) δ 9.29 (s, 1H), 8.49 (d, J = 8.4 Hz, 1H), 8.02 (s, 1H), 7.74 (t, J = 8.0 Hz, 1H), 7.67-7.65 (d, J = 8.8 Hz, 2H), 7.25 (d, J = 7.2 Hz, 2H), 7.05 (d, J = 7.2 Hz, 1H), 4.70-4.67 (m, 1H), 3.78-3.64 (m, 4H), 3.30-3.24 (m, 2H), 2.80 (s, 3H), 2.37-2.30 (m, 4H), 2.22-2.17 (m, 1H), 2.11-2.05 (m, 1H), | LCMS (ES, m/z): 542 [M + H]+. Rt 0.591 min. LCMS (ES, m/z): 542 [M + H]+. Rt 0.599 min. |
| | | 1.69-1.61 (m, 1H), 1.40 (s, 3H), 1.28 (s, | |
| | | 3H), 1.22 (s, 3H), 1.01 (s, 3H) | |
| 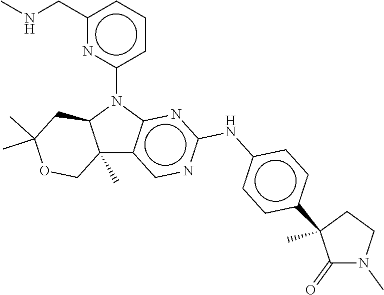 | | | |
| 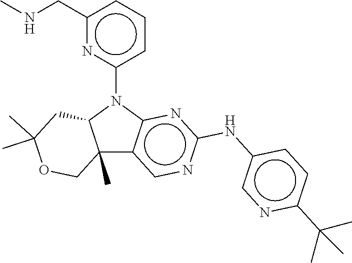 | 81, 82 | 1H NMR (300 MHz, DMSO-d6) δ 9.42 (s, 1H), 8.75 (s, 1H), 8.49 (d, J = 8.4 Hz, 1H), 8.14 (d, J = 9.0 Hz, 1H), 8.05 (s, 1H), 7.76 (t, J = 7.8 Hz, 1H), 7.37 (d, J = 8.7 Hz, 1H), 7.09 (d, J = 7.5 Hz, 1H), 4.73-4.69 (m, 1H), 3.81-3.65 (m, 4H), 2.35 (s, 3H), 2.24-2.18 (m, 1H), 1.69-1.61 (m, 1H), 1.31 (s, 9H), 1.29 (s, 3H), 1.23 (s, 3H), 1.02 (s, 3H). 1H NMR (300 MHz, DMSO-d6) δ 9.42 (s, 1H), 8.75 (s, 1H), 8.49 (d, J = 8.4 Hz, 1H), 8.14 (d, J = 9.0 Hz, 1H), 8.05 (s, 1H), 7.75 (t, J = 7.8 Hz, 1H), 7.37 (d, J = 8.7 Hz, 1H), 7.08 (d, J = 7.5 Hz, 1H), 4.73-4.69 (m, 1H), 3.81-3.65 (m, 4H), 2.35 (s, 3H), 2.29-2.18 (m, 1H), 1.69-1.62 (m, 1H), | LCMS (ES, m/z): 488 [M + H]+. Rt 0.593 min. LCMS (ES, m/z): 488 [M + H]+. Rt 0.587 min. |
| | | 1.31 (s, 9H), 1.29 (s, 3H), 1.23 (s, 3H), | |
| | | 1.02 (s, 3H). | |
| 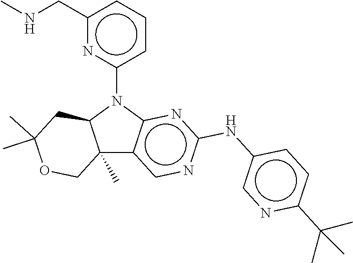 | | | |
| 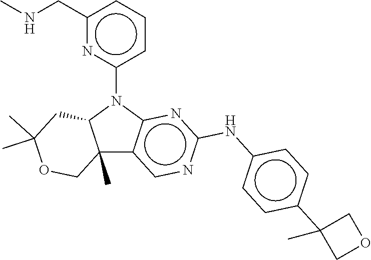 | 83, 84 | 1H NMR (300 MHz, DMSO-d6) δ 9.34 (s, 1H), 8.52 (d, J = 8.4 Hz, 1H), 8.04 (s, 1H), 7.80-7.71 (m, 3H), 7.20 (d, J = 8.4 Hz, 2H), 7.08 (d, J = 7.2 Hz, 1H), 4.82-4.79 (m, 2H), 4.73-4.68 (m, 1H), 4.54-4.51 (m, 2H), 3.80-3.65 (m, 4H), 2.35 (s, 3H), 2.24-2.18 (m, 1H), 1.69-1.62 (m, 4H), 1.29 (s, 3H), 1.23 (s, 3H), 1.02 (s, 3H). 1H NMR (300 MHz, DMSO-d6) δ 9.34 (s, 1H), 8.52 (d, J = 8.4 Hz, 1H), 8.04 (s, 1H), 7.80-7.71 (m, 3H), 7.18 (d, J = 8.4 Hz, 2H), 7.05 (d, J = 7.5 Hz, 1H), 4.82-4.79 (m, 2H), 4.73-4.68 (m, 1H), 4.54-4.51 (m, 2H), 3.80-3.65 (m, 4H), 2.35 (s, 3H), 2.23-2.18 (m, 1H), 1.69-1.61 (m, 4H), 1.29 (s, 3H), 1.23 (s, 3H), 1.02 (s, 3H). | LCMS (ES, m/z): 501 [M + H]+. Rt 0.599 min. LCMS (ES, m/z): 501 [M + H]+. Rt 0.603 min. |
| 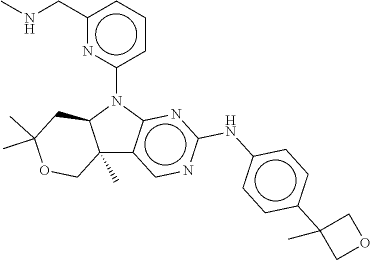 | | | |
| 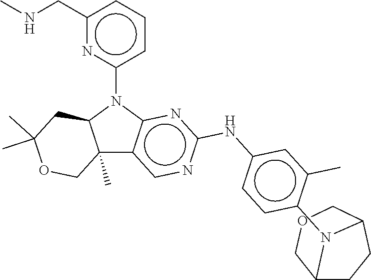 | 85, 86 | 1H NMR (300 MHz, DMSO-d6) δ 9.10 (s, 1H), 8.48 (d, J = 8.4 Hz, 1H), 8.00 (s, 1H), 7.76-7.71(m, 1H), 7.57 (s, 1H), 7.33 (d, J = 7.8 Hz, 1H), 7.05 (d, J = 7.5 Hz, 1H), 6.79 (d, J = 8.7 Hz, 1H), 4.70 (s, 1H), 3.78- 3.63 (m, 6H), 3.61-3.57 (m, 4H), 2.36 (s, 3H), 2.32 (s, 3H), 2.18-2.06 (m, 1H), 1.98-1.90 (m, 4H), 1.66-1.64 (m, 1H), 1.29 (s, 3H), 1.23 (s, 3H), 1.02 (s, 3H). 1H NMR (300 MHz, DMSO-d6) δ 9.12 (s, 1H), 8.48 (d, J = 8.1 Hz, 1H), 8.01 (d, J = 5.4 Hz, 1H), 7.76-7.71 (m, 1H), 7.57 (s, 1H), 7.33 (d, J = 7.8 Hz , 1H), 7.05 (d, J = 7.2 Hz, 1H), 6.79 (d, J = 8.4 Hz, 1H), 4.72-4.68 (m, 1H), 3.84-3.61 (m, 6H), 3.61-3.34 (m, 4H), 2.35 (s, 3H), 2.32 (s, 3H), 2.23-2.18 (m, 1H), 1.92-1.85 (m, | LCMS (ES, m/z): 556 [M+H] +, Rt 0.680 min LCMS (ES, m/z): 556 [M+H] +, Rt 0.682 min |
| | | 4H), 1.67-1.59(m, 1H), 1.28 (s, 3H), 1.23 | |
| | | (s, 3H), 1.04 (s, 3H). | |
| 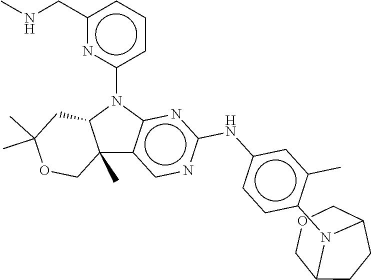 | | | |
| 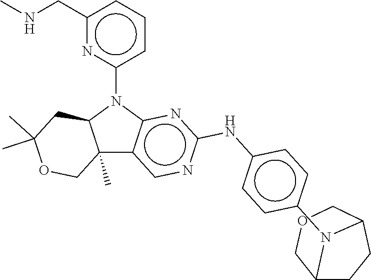 | 87, 88 | 1H NMR (300 MHz, DMSO-d6) δ 9.02 (s, 1H), 8.48 (d, J = 8.1 Hz, 1H), 7.97 (s, 1H), 7.73 (t, J = 7.8 Hz, 1H), 7.51 (d, J = 8.1 Hz, 2H), 7.03 (d, J = 7.5 Hz, 1H), 6.83 (d, J = 7.8 Hz, 2H), 4.67-4.66 (m, 1H), 4.09 (s, 2H), 3.76-3.63 (m, 6H), 3.46-3.43 (m, 2H), 2.34 (s, 3H), 2.28-2.18 (m, 1H), 1.93-1.88 (m, 4H), 1.66-1.59 (m, 1H), 1.27 (s, 3H), 1.22 (s, 3H), 1.03 (s, 3H). 1H NMR (300 MHz, DMSO-d6) δ 9.01 (s, 1H), 8.47 (d, J = 8.7 Hz, 1H), 7.96 (s, 1H), 7.72 (t, J = 7.8 Hz, 1H), 7.50 (d, J = 8.1 Hz, 2H), 7.02 (d, J = 7.5 Hz, 1H), 6.84 (d, J = 8.1 Hz, 2H), 4.66-4.64 (m, 1H), 4.07 (s, 2H), 3.84-3.62 (m, 6H), 3.45-3.42 (m, 2H), 2.33 (s, 3H), 2.27-2.10 (m, 1H), 1.92-1.78 (m, 4H), 1.65-1.53 (m, 1H), | LCMS (ES, m/z): 542 [M + H]+. Rt 0.614 min. LCMS (ES, m/z): 542 [M + H]+. Rt 0.615 min. |
| | | 1.26 (s, 3H), 1.22 (s, 3H), 1.01 (s, 3H). | |
| 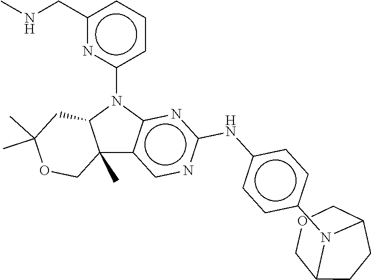 | | | |
| 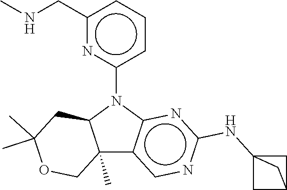 | 89, 90 | 1H NMR (300 MHz, DMSO-d6) δ 8.45 (d, J = 8.4 Hz, 1H), 7.88 (s, 1H), 7.71 (t, J = 7.8 Hz, 1H), 7.60 (s, 1H), 7.01(d, J = 6.0 Hz, 1H), 4.68-4.48 (m, 1H), 3.80-3.50 (m, 4H), 2.45-2.44 (m, 1H), 2.33 (s, 3H), 2.20-2.10 (m, 1H), 2.05 (s, 6H), 1.67- 1.52 (m, 1H), 1.27 (s, 3H), 1.22 (s, 3H), 1.01 (s, 3H). 1H NMR (300 MHz, DMSO-d6) δ 8.45 (d, J = 8.4 Hz, 1H), 7.88 (s, 1H), 7.71 (t, J = 7.8 Hz, 1H), 7.60 (s, 1H), 7.01 (d, J = 6.9 Hz, 1H), 4.68-4.40 (m, 1H), 3.80-3.50 | LCMS (ES, m/z): 421 [M + H]+. Rt 0.587 min. LCMS (ES, m/z): 421 [M + H]+. Rt 0.596 min. |
| | | (m, 4H), 2.45-2.44 (m, 1H), 2.34 (s, 3H), | |
| | | 2.20-2.10 (m, 1H), 2.05 (s, 6H), 1.67- | |
| | | 1.52 (m, 1H), 1.27 (s, 3H), 1.22 (s, 3H), | |
| | | 1.01 (s, 3H). | |
| 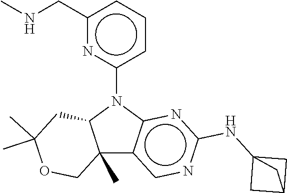 | | | |
| 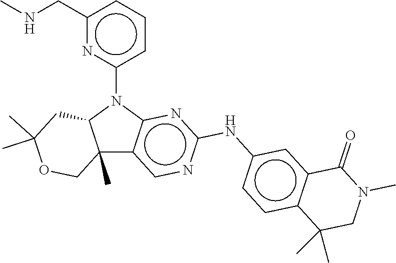 | 91, 92 | 1H NMR (400 MHz, DMSO-d6) δ 9.45 (s, 1H), 8.53 (d, J = 8.4 Hz, 1H), 8.27 (d, J = 2.4 Hz, 1H), 8.04 (s, 1H), 7.85-7.82 (m, 1H), 7.76 (t, J = 7.6 Hz, 1H), 7.32 (d, J = 8.4 Hz, 1H), 7.06 (d, J = 7.6 Hz, 1H), 4.73-4.70 (m, 1H), 3.80-3.65 (m, 4H), 3.32 (s, 2H), 3.07 (s, 3H), 2.35 (s, 3H), 2.23-2.18 (m, 1H), 1.66-1.61 (m, 1H), 1.29 (s, 3H), 1.28 (s, 6H), 1.23 (s, 3H), 1.02 (s, 3H). 1H NMR (400 MHz, DMSO-d6) δ 9.45 (s, 1H), 8.53 (d, J = 8.4 Hz, 1H), 8.27 (d, J = 2.4 Hz, 1H), 8.04 (s, 1H), 7.85-7.82 (m, 1H), 7.76 (t, J = 8.0 Hz, 1H), 7.32 (d, J = 8.4 Hz, 1H), 7.06 (d, J = 7.2 Hz, 1H), 4.73-4.70 (m, 1H), 3.80-3.65 (m, 4H), | LCMS (ES, m/z): 542 [M + H]+. Rt 0.612 min. LCMS (ES, m/z): 542 [M + H]+. Rt 0.608 min. |
| | | 3.34-3.32 (m, 2H), 3.07 (s, 3H), 2.35 (s, | |
| | | 3H), 2.23-2.18 (m, 1H), 1.66-1.61 (m, | |
| | | 1H), 1.29 (s, 3H), 1.28 (s, 6H), 1.23 (s, | |
| | | 3H), 1.02 (s, 3H). | |
| 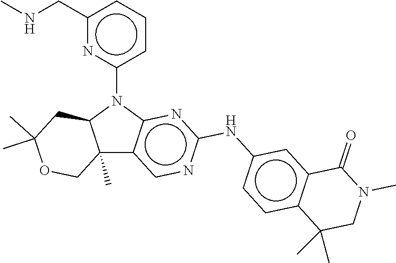 | | | |
| 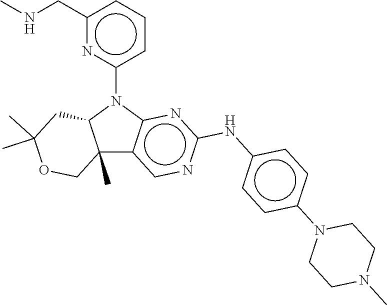 | 93, 94 | 1H NMR (300 MHz, DMSO-d6) δ 9.08 (s, 1H), 8.48 (d, J = 8.1 Hz, 1H), 7.98 (s, 1H), 7.74 (t, J = 8.1 Hz, 1H), 7.55 (d, J = 9.0 Hz, 2H), 7.03 (d, J = 7.2 Hz, 1H), 6.89 (d, J = 9.0 Hz, 2H), 4.76-4.68 (m, 1H), 3.82- 3.58 (m, 4H), 3.18-2.95 (m, 4H), 2.33 (s, 3H), 2.35-2.20 (m, 8H), 1.70-1.55 (m, 1H), 1.26 (s, 3H), 1.22 (s, 3H), 1.01 (s, 3H). 1H NMR (300 MHz, DMSO-d6) δ 9.07 (s, 1H), 8.48 (d, J = 8.4 Hz, 1H), 7.98 (s, 1H), 7.74 (t, J = 7.8 Hz, 1H), 7.54 (d, J = 9.0 Hz, 2H), 7.03 (d, J = 7.2 Hz, 1H), 6.89 (d, J = 9.0 Hz, 2H), 4.79-4.55 (m, 1H), 3.87- 3.53 (m, 4H), 3.15-2.90 (m, 4H), 2.33 (s, 3H), 2.31-2.20 (m, 8H), 1.70-1.55 (m, 1H), 1.27 (s, 3H), 1.22 (s, 3H), 1.01 (s, 3H). | LCMS (ES, m/z): 529 [M + H]+. Rt 0.660 min. LCMS (ES, m/z): 529 [M + H]+. Rt 0.652 min. |
| 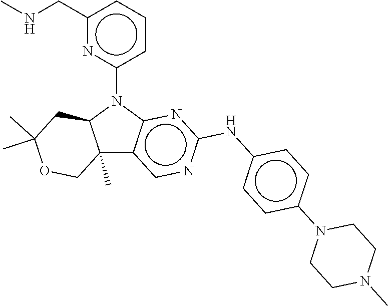 | | | |
| 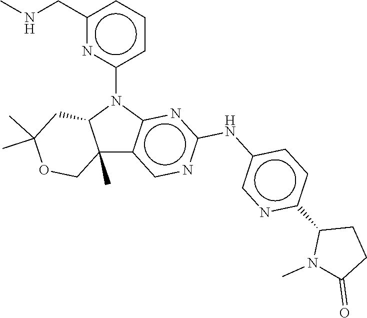 | 95, 96 | 1H NMR (300 MHz, DMSO-d6) δ 9.58 (s, 1H), 8.85 (s, 1H), 8.45 (d, J = 8.4 Hz, 1H), 8.29-8.23 (m, 1H), 8.08 (s, 1H), 7.80- 7.72 (m, 1H), 7.27 (d, J = 8.7 Hz, 1H), 7.08 (d, J = 7.5 Hz, 1H), 4.77-4.58 (m, 2H), 3.84-3.63 (m, 4H), 2.48-2.15 (m, 10H), 1.98-1.86 (m, 1H), 1.72-1.59 (m, 1H), 1.29 (s, 3H), 1.23 (s, 3H), 1.01 (s, 3H). 1H NMR (300 MHz, DMSO-d6) δ 9.58 (s, 1H), 8.85 (s, 1H), 8.45 (d, J-8.4 Hz, 1H), 8.31-8.21 (m, 1H), 8.08 (s, 1H), 7.76 (m, 1H), 7.27 (d, J-8.4 Hz, 1H), 7.08 (d, J-7.5 Hz, 1H), 4.77-4.60 (m, 2H), 3.84-3.62 (m, 4H), 2.48-2.14 (m, 10H), 1.98-1.86 (m, 1H), 1.73-1.60 (m, 1H), 1.29 (s, 3H), 1.23 (s, 3H), 1.02 (s, 3H). | LCMS (ES, m/z): 529[M + H]+, Rt 0.505 min LCMS (ES, m/z): 529[M + H]+, Rt 0.507 min |
| 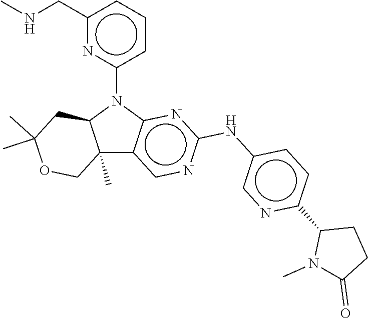 | | | |
| 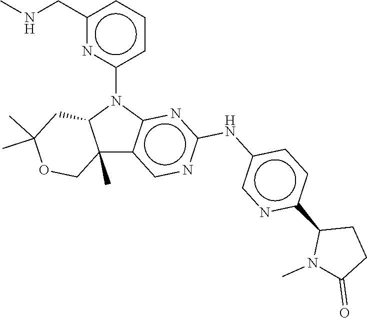 | 97, 98 | 1H NMR (300 MHz, DMSO-d6) δ 9.58 (s, 1H), 8.85 (s, 1H), 8.45 (d, J = 8.1 Hz, 1H), 8.26 (d, J = 8.1 Hz, 1H), 8.08 (s, 1H), 7.79- 7.74 (m, 1H), 7.27 (d, J = 8.4 Hz, 1H), 7.08 (d, J = 7.5 Hz, 1H), 4.74-4.61 (m, 2H), 3.81-3.65 (m, 4H), 2.48 (s, 3H), 2.42-2.26 (m, 6H), 2.25-2.14 (m, 1H), 1.98-1.88 (m, 1H), 1.76-1.67 (m, 1H), 1.30 (s, 3H), 1.23 (s, 3H), 1.02 (s, 3H). 1H NMR (300 MHz, DMSO-d6) δ 9.58 (s, 1H), 8.85 (s, 1H), 8.45 (d, J = 7.5 Hz, 1H), 8.26 (d, J = 7.8 Hz, 1H), 8.08 (s, 1H), 7.79- 7.74 (m, 1H), 7.27 (d, J = 8.7 Hz, 1H), 7.08 (d, J = 7.2 Hz, 1H), 4.74-4.61 (m, 2H), 3.81-3.65 (m, 4H), 2.73 (s, 3H), 2.36-2.33 (m, 6H), 2.22-2.17 (m, 1H), 1.96-1.91 (m, 1H), 1.69-1.62 (m, 1H), 1.30 (s, 3H), 1.23 (s, 3H), 1.02 (s, 3H). | LCMS (ES, m/z): 529 [M + H]+, Rt 0.528 min LCMS (ES, m/z): 529 [M + H]+, Rt 0.535 min |
| 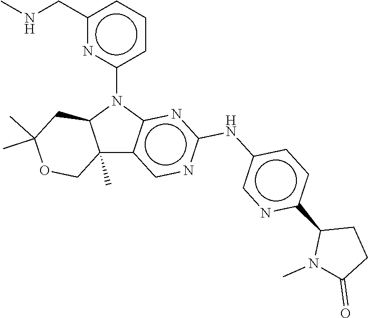 | | | |
| 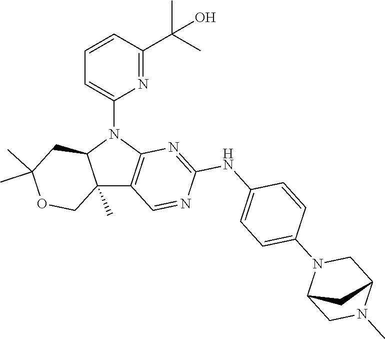 | 413, 414 | 1H NMR (300 MHz, DMSO-d6) δ 8.94 (s, 1H), 8.49 (d, J = 8.7 Hz, 1H), 7.94 (s, 1H), 7.74 (t, J = 7.8 Hz, 1H), 7.45 (d, J = 8.7 Hz, 2H), 7.28 (d, J = 7.5 Hz, 1H), 6.55 (d, J = 9.0 Hz, 2H), 5.17 (s, 1H), 4.66- 4.56 (m, 1H), 4.26 (s, 1H), 3.82-3.62 (m, 2H), 3.48-3.42 (m, 1H), 3.17-3.08 (m, 1H), 2.84-2.74 (m, 1H), 2.61-2.52 (m, 2H), 2.28 (s, 3H), 2.26-2.20 (m, 1H), 1.92-1.71 (m, 2H), 1.61-1.52 (m, 1H), 1.46 (s, 6H), 1.29 (s, 3H), 1.24 (s, 3H), 1.04 (s, 3H). 1H NMR (300 MHz, DMSO-d6) δ 8.94 (s, 1H), 8.51 (d, J = 8.7 Hz, 1H), 7.94 (s, 1H), 7.75 (t, J = 7.8 Hz, 1H), 7.45 (d, J = 8.7 Hz, 2H), 7.28 (d, J = 7.5 Hz, 1H), 6.55 (d, J = 9.0 Hz, 2H), 5.17 (s, 1H), 4.66- 4.56 (m, 1H), 4.27 (s, 1H), 3.82-3.62 (m, 2H), 3.48-3.42 (m, 1H), 3.17-3.08 (m, 1H), 2.84-2.74 (m, 1H), 2.61-2.52 (m, 2H), 2.28 (s, 3H), 2.26-2.20 (m, 1H), | LCMS (ES, m/z): 556 [M + H]+; RT: 0.662 min. LCMS (ES, m/z): 556 [M + H]+; RT: 0.66 min. |
| | | 1.92-1.71 (m, 2H), 1.61-1.52 (m, 1H), | |
| | | 1.46 (s, 6H), 1.29 (s, 3H), 1.24 (s, 3H), | |
| | | 1.05 (s, 3H). | |
| 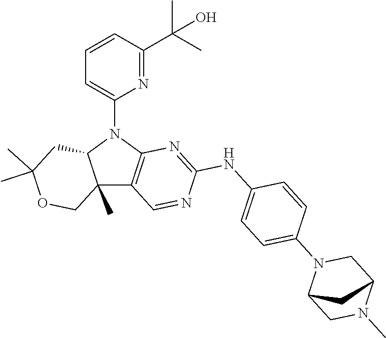 | | | |
| 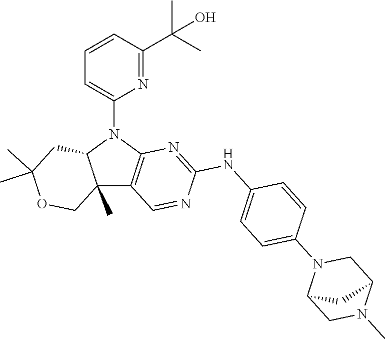 | 415, 416 | 1H NMR (300 MHz, DMSO-d6) δ 8.94 (s, 1H), 8.49 (d, J = 8.4 Hz, 1H), 7.94 (s, 1H), 7.73 (t, J = 8.1 Hz, 1H), 7.45 (d, J = 8.4 Hz, 2H), 7.28 (d, J = 7.5 Hz, 1H), 6.55 (d, J = 8.7 Hz, 2H), 5.17 (s, 1H), 4.66- 4.56 (m, 1H), 4.26 (s, 1H), 3.82-3.62 (m, 2H), 3.48-3.42 (m, 1H), 3.17-3.08 (m, 1H), 2.84-2.74 (m, 1H), 2.61-2.52 (m, 2H), 2.29 (s, 3H), 2.29-2.18 (m, 1H), 1.94-1.74 (m, 2H), 1.64-1.50 (m, 1H), 1.46 (s, 6H), 1.29 (s, 3H), 1.24 (s, 3H), 1.04 (s, 3H). 1H NMR (300 MHz, DMSO-d6) δ 8.94 (s, 1H), 8.49 (d, J = 8.4 Hz, 1H), 7.94 (s, 1H), 7.73 (t, J = 8.1 Hz, 1H), 7.45 (d, J = 8.7 Hz, 2H), 7.28 (d, J = 7.5 Hz, 1H), 6.61-6.49 (m, 2H), 5.17 (s, 1H), 4.66- 4.56 (m, 1H), 4.26 (s, 1H), 3.81-3.63 (m, 2H), 3.50-3.44 (m, 1H), 3.18-3.09 (m, 1H), 2.84-2.75 (m, 1H), 2.63-2.53 (m, 2H), 2.30 (s, 3H), 2.27-2.17 (m, 1H), | LCMS (ES, m/z): 556 [M + H]+; RT: 1.257 min. LCMS (ES, m/z): 556 [M + H]+; RT: 1.264 min. |
| | | 1.94-1.75 (m, 2H), 1.64-1.51 (m, 1H), | |
| | | 1.46 (s, 6H), 1.29 (s, 3H), 1.24 (s, 3H), | |
| | | 1.04 (s, 3H). | |
| 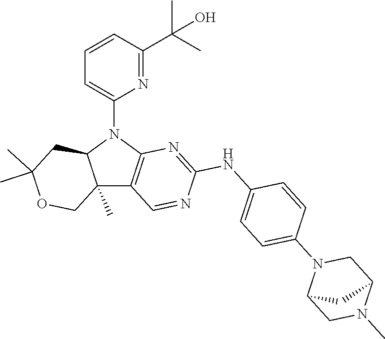 | | | |
| 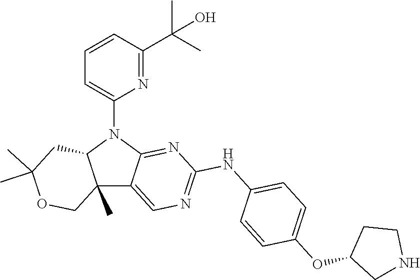 [SMILES: CC1(C)C[C@@H]2N(c3cccc(C(C)(C)O)n3)c3nc(Nc4ccc(O[C@@H]5CCNC5)cc4)ncc3[C@]2(C)CO1] | 417 418 | 1H NMR (300 MHz, DMSO-d6) δ 9.14 (s, 1H), 8.47 (d, J = 8.4 Hz, 1H), 7.97 (s, 1H), 7.78-7.58 (m, 3H), 7.29 (d, J = 7.5 Hz, 1H), 6.85 (d, J = 8.7 Hz, 2H), 5.40- 4.86(m, 1H), 4.81 (s, 1H), 4.65-4.60 (m, 1H), 3.79-3.75 (m, 1H), 3.68-3.64 (m, 1H), 3.09-3.03 (m, 1H), 2.97-2.76 (m, 2H), 2.51 (s, 1H), 2.28-2.22 (m, 1H), 2.06-1.97 (m, 1H), 1.80-1.75 (m, 1H), 1.63-1.55 (m, 1H), 1.46 (s, 6H), 1.29 (s, 3H), 1.23 (s, 3H), 1.04 (s, 3H). 1H NMR (300 MHz, DMSO-d6) δ 9.13 (s, 1H), 8.47 (d, J = 8.4 Hz, 1H), 7.97 (s, 1H), 7.75-7.73 (m, 1H), 7.59 (d, J = 8.7 Hz, 2H), 7.29 (d, J = 7.5 Hz, 1H), 6.85 (d, J = 9.0 Hz, 2H), 5.40-4.87(m, 1H), 4.80 (s, 1H), 4.65-4.60 (m, 1H), 3.79-3.75 (m, | LCMS (ES, m/z): 531 [M + H]+. Rt 0.660 min. LCMS (ES, m/z): 531 [M + H]+. Rt 0.655 min. |
| | | 1H), 3.68-3.64 (m, 1H), 3.08-3.02 (m, | |
| | | 1H), 2.96 -2.88 (m, 1H), 2.83-2.75 (m, | |
| | | 1H), 2.50 (s, 1H), 2.27-2.21 (m, 1H), | |
| | | 2.06-1.96 (m, 1H), 1.79-1.75 (m, 1H), | |
| | | 1.62-1.55 (m, 1H), 1.46 (s, 6H), 1.29 (s, | |
| | | 3H), 1.23 (s, 3H), 1.04 (s, 3H). | |
| 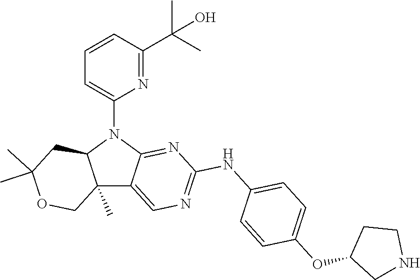 [SMILES: CC1(C)C[C@H]2N(c3cccc(C(C)(C)O)n3)c3nc(Nc4ccc(O[C@@H]5CCNC5)cc4)ncc3[C@@]2(C)CO1] | | | |
| 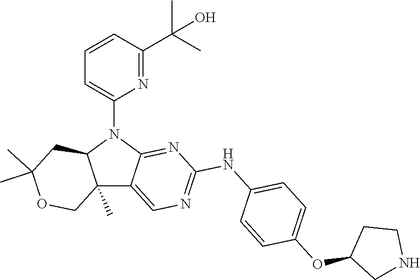 [SMILES: CC1(C)C[C@H]2N(c3cccc(C(C)(C)O)n3)c3nc(Nc4ccc(O[C@H]5CCNC5)cc4)ncc3[C@@]2(C)CO1] | 419, 420 | 1H NMR (300 MHz, DMSO-d6) δ 9.13 (s, 1H), 8.46 (d, J = 8.4 Hz, 1H), 7.96 (s, 1H), 7.75 (t, J = 7.8 Hz, 1H), 7.58 (d, J = 8.7 Hz, 2H), 7.28 (d, J = 7.5 Hz, 1H), 6.86-6.81 (m, 2H), 5.16 (s, 1H), 4.79- 4.64 (m, 1H), 4.62-4.59 (m, 1H), 3.78- 3.64 (m, 2H), 3.07-2.76 (m, 4H), 2.27- 2.20 (m, 1H), 1.98-1.90 (m, 1H), 1.78- 1.74 (m, 1H), 1.62-1.58 (m, 1H), 1.45 (s, 6H), 1.29 (s, 3H), 1.23 (s, 3H), 1.04 (s, 3H). 1H NMR (300 MHz, DMSO-d6) δ 9.14 (s, 1H), 8.47 (d, J = 8.1 Hz, 1H), 7.97 (s, 1H), 7.75 (t, J = 7.8 Hz, 1H), 7.59 (d, J = 9.0 Hz, 2H), 7.29 (d, J = 7.5 Hz, 1H), 6.86-6.81 (m, 2H), 5.16 (s, 1H), 4.79- 4.64 (m, 1H), 4.62-4.59 (m, 1H), 3.78- | LCMS (ES, m/z): 531 [M + H]+, Rt 0.644 min. LCMS (ES, m/z): 531 [M + H]+, Rt 0.646 min. |
| | | 3.64 (m, 2H), 3.07-2.76 (m, 4H), 2.27- | |
| | | 2.20 (m, 1H), 1.98-1.90 (m, 1H), 1.78- | |
| | | 1.74 (m, 1H), 1.62-1.58 (m, 1H), 1.46 (s, | |
| | | 6H), 1.29 (s, 3H), 1.23 (s, 3H), 1.04 (s, | |
| | | 3H) | |
| 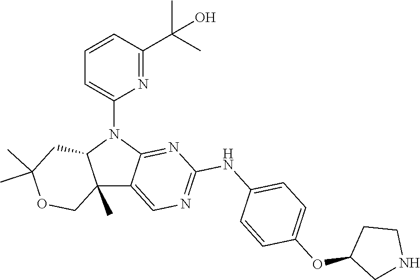 [SMILES: CC1(C)C[C@@H]2N(c3cccc(C(C)(C)O)n3)c3nc(Nc4ccc(O[C@H]5CCNC5)cc4)ncc3[C@]2(C)CO1] | | | |
| 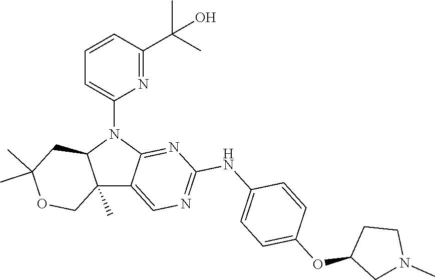 | 421, 422 | 1H NMR (300 MHz, DMSO-d6) δ 9.13 (s, 1H), 8.46 (d, J = 8.4 Hz, 1H), 7.97 (s, 1H), 7.75 (t, J = 7.8 Hz, 1H), 7.68 (d, J = 9.0 Hz, 2H), 7.28 (d, J = 7.5 Hz, 1H), 6.82 (d, J = 9.0 Hz, 2H), 5.16 (s, 1H), 4.83- 4.79 (m, 1H), 4.64-4.59 (m, 1H), 3.78 - 3.64 (m, 2H), 2.80-2.75 (m, 1H), 2.65- 2.55 (m, 2H), 2.40- 2.17 (m, 6H), 1.80- 1.76 (m, 1H), 1.60-1.56 (m, 1H), 1.46 (s, 6H), 1.29 (s, 3H), 1.23 (s, 3H), 1.04 (s, 3H). 1H NMR (300 MHz, DMSO-d6) δ 9.13 (s, 1H), 8.46 (d, J = 8.4 Hz, 1H), 7.97 (s, 1H), 7.75 (t, J = 7.8 Hz, 1H), 7.68 (d, J = 9.0 Hz, 2H), 7.28 (d, J = 7.5 Hz, 1H), 6.82 (d, J = 9.0 Hz, 2H), 5.16 (s, 1H), 4.83- 4.79 (m, 1H), 4.64-4.59 (m, 1H), 3.78 - | LCMS (ES, m/z): 545 [M + H]+, Rt 0.633 min. LCMS (ES, m/z): 545 [M + H]+, Rt 0.649 min. |
| | | 3.64 (m, 2H), 2.80-2.75 (m, 1H), 2.65- | |
| | | 2.55 (m, 2H), 2.40- 2.17 (m, 6H), 1.80- | |
| | | 1.76 (m, 1H), 1.60-1.56 (m, 1H), 1.46 (s, | |
| | | 6H), 1.29 (s, 3H), 1.23 (s, 3H), 1.04 (s, | |
| | | 3H). | |
| 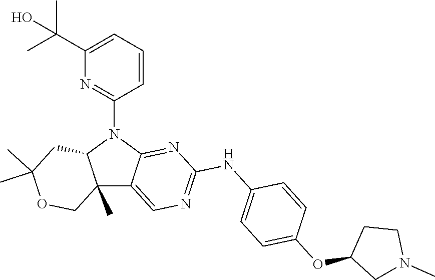 | | | |
| 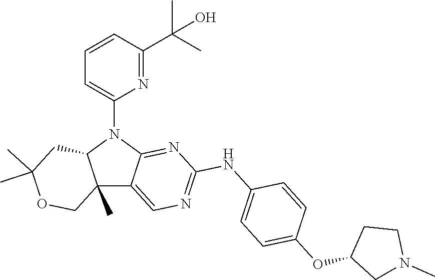 | 423, 424 | 1H NMR (300 MHz, DMSO-d6) δ 9.13 (s, 1H), 8.47 (d, J = 8.4 Hz, 1H), 7.97 (s, 1H), 7.78-7.73 (m, 1H), 7.59 (d, J = 8.7 Hz, 2H), 7.29 (d, J = 7.8 Hz, 1H), 6.82 (d, J = 8.7 Hz, 2H), 5.15 (s, 1H), 4.81-4.79 (m, 1H), 4.65-4.60 (m, 1H), 3.79-3.64 (m, 2H), 2.80-2.75 (m, 1H), 2.68-2.56 (m, 2H), 2.40-2.32 (m, 1H), 2.26-2.23 (m, 5H), 1.79-1.74 (m, 1H), 1.67-1.55 (m, 1H), 1.46 (s, 6H), 1.29 (s, 3H), 1.23 (s, 3H), 1.04 (s, 3H). LCMS (ES, m/z): 545 [M + H]+, Rt 0.659 min. 1H NMR (300 MHz, DMSO-d6) δ 9.13 (s, 1H), 8.47 (d, J = 8.4 Hz, 1H), 7.97 (s, 1H), 7.78-7.73 (m, 1H), 7.59 (d, J = 8.7 Hz, 2H), 7.29 (d, J = 7.5 Hz, 1H), 6.82 (d, J = 9.0 Hz, 2H), 5.15 (s, 1H), 4.82 (s, 1H), | LCMS (ES, m/z): 545 [M + H]+, Rt 0.659 min. LCMS (ES, m/z): 545 [M + H]+, Rt 0.652 min. |
| | | 4.64-4.60 (m, 1H), 3.79-3.64 (m, 2H), | |
| | | 2.81-2.76 (m, 1H), 2.68-2.56 (m, 2H), | |
| | | 2.41-2.32 (m, 1H), 2.29-2.21 (m, 5H), | |
| | | 1.80-1.74 (m, 1H), 1.62-1.55 (m, 1H), | |
| | | 1.46 (s, 6H), 1.29 (s, 3H), 1.23 (s, 3H), | |
| | | 1.04 (s, 3H). LCMS (ES, m/z): 545 | |
| | | [M + H]+, Rt 0.652 min. | |
| 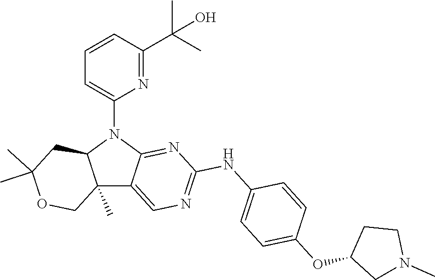 | | | |
| 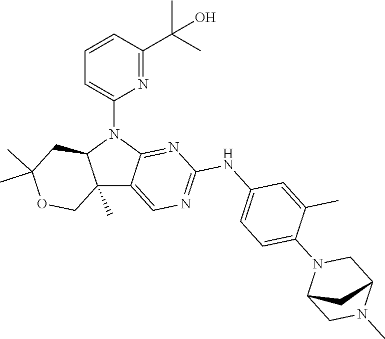 | 425, 424 | 1H NMR (300 MHz, DMSO-d6) δ 9.03 (s, 1H), 8.47 (d, J = 8.4 Hz, 1H), 7.96 (s, 1H), 7.72 (t, J = 7.8 Hz, 1H), 7.48-4.73 (m, 1H), 7.28 (d, J = 7.5 Hz, 2H), 6.74 (d, J = 8.7 Hz, 1H), 5.15 (s, 1H), 4.64-4.59 (m, 1H), 3.92 (s, 1H), 3.78-3.63 (m, 2H), 3.38-3.32 (m, 1H), 3.26-3.19 (m, 2H), 2.79-2.72 (m, 2H), 2.32-2.21 (m, 7H), 1.83-1.80 (m, 1H), 1.74-1.71 (m, 1H), 1.61-1.53 (m, 1H), 1.45 (s, 6H), 1.28 (s, 3H), 1.23 (s, 3H), 1.03 (s, 3H). 1H NMR (300 MHz, DMSO-d6) δ 9.01 (s, 1H), 8.47 (d, J = 8.4 Hz, 1H), 7.96 (s, 1H), 7.72 (t, J = 7.8 Hz, 1H), 7.48-4.73 (m, 1H), 7.28 (d, J = 7.5 Hz, 2H), 6.74 (d, J = 8.7 Hz, 1H), 5.15 (s, 1H), 4.64-4.59 (m, 1H), 3.92 (s, 1H), 3.78-3.63 (m, 2H), 3.38-3.32 (m, 1H), 3.26-3.19 (m, 2H), 2.79-2.72 (m, 2H), 2.32-2.21 (m, 7H), 1.83-1.80 (m, 1H), 1.74-1.71 (m, 1H), 1.61-1.53 (m, 1H), 1.45 (s, 6H), 1.28 (s, | LCMS (ES, m/z): 570 [M + H]+, Rt 0.643 min. LCMS (ES, m/z): 570 [M + H]+, Rt 0.641 min. |
| | | 3H), 1.23 (s, 3H), 1.03 (s, 3H). | |
| 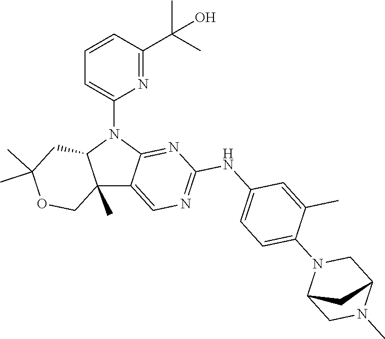 | | | |
| 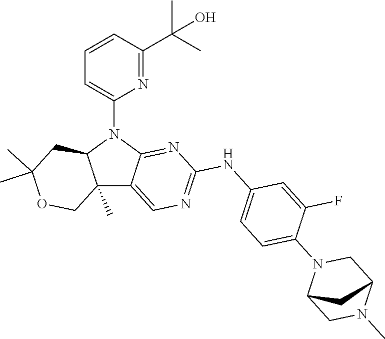 | 427, 428 | 1H NMR (300 MHz, DMSO-d6) δ 9.19 (s, 1H), 8.46 (d, J = 8.1 Hz, 1H), 7.99 (s, 1H), 7.76-7.61 (m, 2H), 7.30 (d, J = 7.5 Hz, 1H), 7.23 (d, J = 8.7 Hz, 1H), 6.72- 6.65 (m, 1H), 5.16 (s, 1H), 4.64-4.60 (m, 1H), 4.18 (s, 1H), 3.81-3.62 (m, 2H), 3.49-3.33 (m, 2H), 3.24-3.15 (m, 1H), 2.79-2.71 (m, 1H), 2.68-2.57 (m, 1H), 2.30-2.21 (m, 4H), 1.84-1.69 (m, 2H), 1.63-1.46 (m, 7H), 1.29 (s, 3H), 1.23 (s, 3H), 1.04 (s, 3H). 1H NMR (300 MHz, DMSO-d6) δ 9.19 (s, 1H), 8.46 (d, J = 8.7 Hz, 1H), 7.99 (s, 1H), 7.76-7.61 (m, 2H), 7.31-7.21 (m, 2H), 6.72-6.65 (m, 1H), 5.16 (s, 1H), 4.64-4.60 (m, 1H), 4.18 (s, 1H), 3.80- 3.64 (m, 2H), 3.43-3.33 (m, 2H), 3.21- 3.17 (m, 1H), 2.76-2.61 (m, 2H), 2.26- 2.21 (m, 4H), 1.83-1.70 (m, 2H), 1.63- 1.55 (m, 1H), 1.46 (s, 6H), 1.29 (s, 3H), 1.23 (s, 3H), 1.04 (s, 3H). | LCMS (ES, m/z): 574[M + H]+, Rt 0.647 min. LCMS (ES, m/z): 574[M + H]+, Rt 0.642 min. |
| 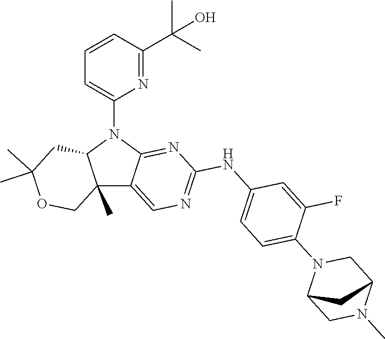 | | | |
| 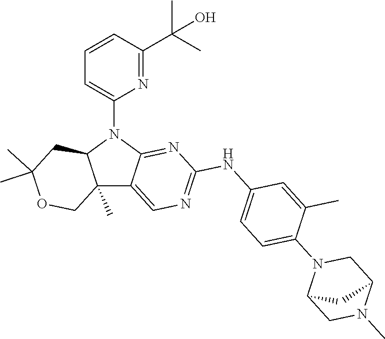 | 429, 430 | 1H NMR (300 MHz, DMSO-d6) δ 9.03 (s, 1H), 8.47 (d, J = 8.4 Hz, 1H), 7.96 (s, 1H), 7.73 (t, J = 8.1 Hz, 1H), 7.48 (d, J = 2.4 Hz, 1H), 7.28 (d, J = 7.8 Hz, 2H), 6.76 (d, J = 8.7 Hz, 1H), 5.16 (s, 1H), 4.67- 4.56 (m, 1H), 3.97-3.91 (m, 1H), 3.81- 3.44 (m, 3H), 2.84-2.78 (m, 2H), 2.37 (s, 3H), 2.30-2.24 (m, 2H), 2.21 (s, 3H), 1.92-1.72 (m, 2H), 1.65-1.51 (m, 1H), 1.46 (s, 6H), 1.33-1.19 (m, 7H), 1.04 (s, 3H). 1H NMR (300 MHz, DMSO-d6) δ 9.03 (s, 1H), 8.47 (d, J = 8.4 Hz, 1H), 7.96 (s, 1H), 7.73 (t, J = 8.1 Hz, 1H), 7.49 (d, J = 2.4 Hz, 1H), 7.34-7.24 (m, 2H), 6.77 (d, J = 8.7 Hz, 1H), 5.16 (s, 1H), 4.67-4.56 (m, 1H), 3.95 (s, 1H), 3.80-3.46 (m, 3H), 2.86-2.80 (m, 2H), 2.39 (s, 3H), 2.29- 2.25 (m, 2H), 2.21 (s, 3H), 1.92-1.74 (m, 2H), 1.64-1.51 (m, 1H), 1.46 (s, 6H), 1.33-1.20 (m, 7H), 1.04 (s, 3H). | LCMS (ES, m/z): 570 [M + H]+; RT: 0.648 min. LCMS (ES, m/z): 570 [M + H]+; RT: 0.640 min. |
| 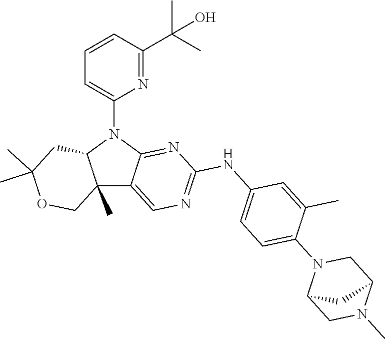 | | | |
| 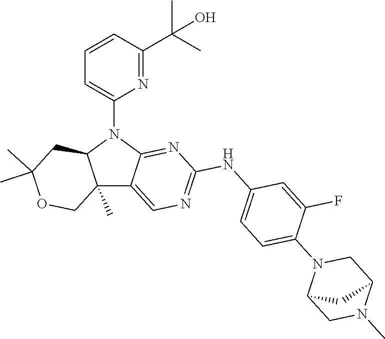 | 431, 432 | 1H NMR (300 MHz, DMSO-d6) δ 9.20 (s, 1H), 8.47 (d, J = 8.4 Hz, 1H), 7.99 (s, 1H), 7.77-7.71 (m, 1H), 7.68-7.62 (m, 1H), 7.30 (d, J = 7.5 Hz, 1H), 7.25-7.22 (m, 1H), 6.72-6.65 (m, 1H), 5.17 (s, 1H), 4.65-4.60 (m, 1H), 4.18 (s, 1H), 3.79- 3.75 (m, 1H), 3.68-3.64 (m, 1H), 3.44- 3.40 (m, 2H), 3.21-3.17 (m, 1H), 2.76- 2.73 (m, 1H), 2.69-2.61 (m, 1H), 2.31- 2.19 (m, 4H), 1.85-1.77 (m, 1H), 1.76- 1.65 (m, 1H), 1.63-1.55 (m, 1H), 1.46 (s, 6H), 1.29 (s, 3H), 1.23 (s, 3H), 1.04 (s, 3H). 1H NMR (300 MHz, DMSO-d6) δ 9.20 (s, 1H), 8.47 (d, J = 8.1 Hz, 1H), 7.99 (s, 1H), 7.76-7.62 (m, 2H), 7.31-7.22 (m, 2H), 6.72-6.65 (m, 1H), 5.17 (s, 1H), 4.65-4.60 (m, 1H), 4.18 (s, 1H), 3.79- 3.75 (m, 1H), 3.68-3.64 (m, 1H), 3.44- 3.41 (m, 2H), 3.21-3.17 (m, 1H), 2.76- 2.73 (m, 1H), 2.69-2.61 (m, 1H), 2.32- | LCMS (ES, m/z): 574 [M + H]+, Rt 0.639 min. LCMS (ES, m/z): 574 [M + H]+, Rt 0.642 min. |
| | | 2.15 (m, 4H), 1.88-1.79 (m, 1H), 1.78- | |
| | | 1.69 (m, 1H), 1.63-1.55 (m, 1H), 1.46 (s, | |
| | | 6H), 1.29 (s, 3H), 1.23 (s, 3H), 1.04 (s, | |
| | | 3H). | |
| 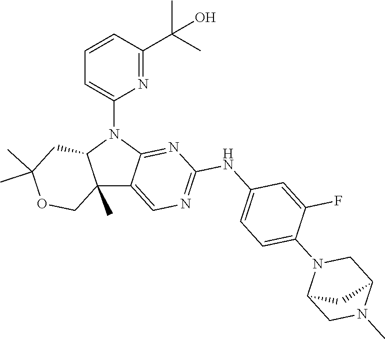 | | | |
| 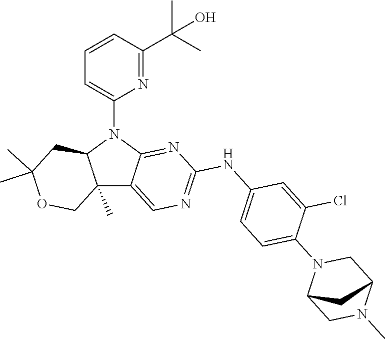 | 433, 434 | 1H NMR (400 MHz, DMSO-d6) δ 9.27 (s, 1H), 8.45 (d, J = 8.4 Hz, 1H), 8.00 (s, 1H), 7.92 (d, J = 2.4 Hz, 1H), 7.76 (t, J = 8.0 Hz, 1H), 7.38-7.29 (m, 2H), 6.91 (d, J = 8.8 Hz, 1H), 5.16 (s, 1H), 4.65-4.61 (m, 1H), 4.13 (s, 1H), 3.78-3.75 (m, 1H), 3.67-3.64 (m, 1H), 3.51-3.49 (m, 1H), 3.43-3.32 (m, 2H), 2.83-2.67 (m, 2H), 2.37-2.33 (m, 3H), 2.26-2.21 (m, 1H), 1.96-1.87 (m, 1H), 1.77-1.74 (m, 1H), 1.61-1.56 (m, 1H), 1.45 (s, 6H), 1.29 (s, 3H), 1.23 (s, 3H), 1.03 (s, 3H). 1H NMR (400 MHz, DMSO-d6) δ 9.26 (s, 1H), 8.45 (d, J = 8.0 Hz, 1H), 8.00 (s, 1H), 7.91 (d, J = 2.4 Hz, 1H), 7.76 (t, J = 8.0 Hz, 1H), 7.37-7.28 (m, 2H), 6.89 (d, J = 8.8 Hz, 1H), 5.16 (s, 1H), 4.65-4.61 (m, 1H), 4.10 (s, 1H), 3.78-3.64 (m, 2H), 3.49-3.47 (m, 1H), 3.45-3.38 (m, 1H), 3.29-3.27 (m, 1H), 2.80-2.67 (m, 2H), 2.31 (s, 3H), 2.26-2.21 (m, 1H), 1.84- | LCMS (ES, m/z): 590 [M + H]+. Rt 0.571 min. LCMS (ES, m/z): 590 [M + H]+. Rt 0.556 min. |
| | | 1.82 (m, 1H), 1.72-1.70 (m, 1H), 1.62- | |
| | | 1.56 (m, 1H), 1.45 (s, 6H), 1.29 (s, 3H), | |
| | | 1.23 (s, 3H), 1.03 (s, 3H). | |
| 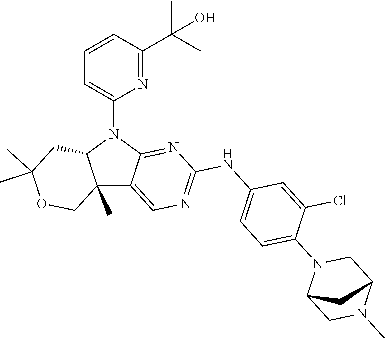 | | | |
| 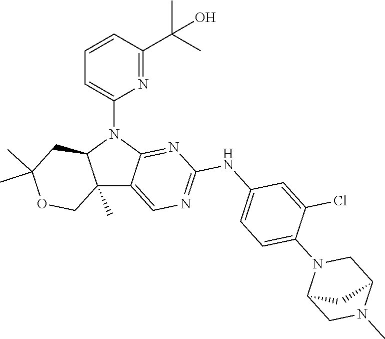 | 435, 436 | 1H NMR (300 MHz, DMSO-d6) δ 9.26 (s, 1H), 8.65-8.29 (m, 1H), 8.00 (s, 1H), 7.95-7.85 (m, 1H), 7.76 (t, J = 8.1 Hz, 1H), 7.65-7.19 (m, 2H), 7.01-6.75(m, 1H), 5.17 (s, 1H), 4.79-4.45 (m, 1H), 4.15-4.02 (m, 1H), 3.86-3.72 (m, 1H), 3.72-3.65 (m, 1H), 3.53-3.35 (m, 2H), 3.28-3.24 (m, 1H), 2.80-2.74 (m, 2H), 2.37-2.22 (m, 4H), 1.85-1.79 (m, 1H), 1.76-1.67 (m, 1H), 1.66-1.52 (m, 1H), 1.46 (s, 6H), 1.29 (s, 3H), 1.23 (s, 3H), 1.03 (s, 3H). H NMR (300 MHz, DMSO-d6) δ 9.26 (s, 1H), 8.65-8.29 (m, 1H), 8.00 (s, 1H), 7.95-7.85 (m, 1H), 7.76 (t, J = 8.1 Hz, 1H), 7.65-7.30 (m, 1H), 7.30-7.15 (m, 1H), 7.01-6.75(m, 1H), 5.17 (s, 1H), 4.79-4.45 (m, 1H), 4.25-4.02 (m, 1H), 3.86-3.72 (m, 1H), 3.72-3.65 (m, 1H), 3.53-3.35 (m, 2H), 3.28-3.24 (m, 1H), 2.80-2.74 (m, 2H), 2.37-2.28 (m, | LCMS (ES, m/z): 590 [M + H]+. Rt 0.661 min. LCMS (ES, m/z): 590 [M + H]+. Rt 0.658 min. |
| | | 3H), 2.28-2.18 (m, 1H), 1.90-1.79 (m, | |
| | | 1H), 1.76-1.67 (m, 1H), 1.66-1.52 (m, | |
| | | 1H), 1.46 (s, 6H), 1.29 (s, 3H), 1.23 (s, | |
| | | 3H), 1.03 (s, 3H). | |
| 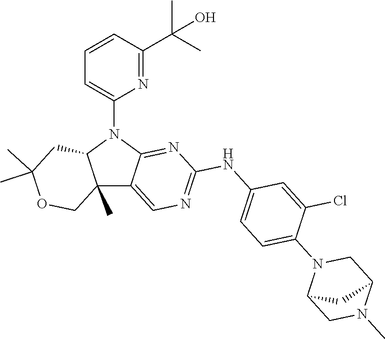 | | | |
| 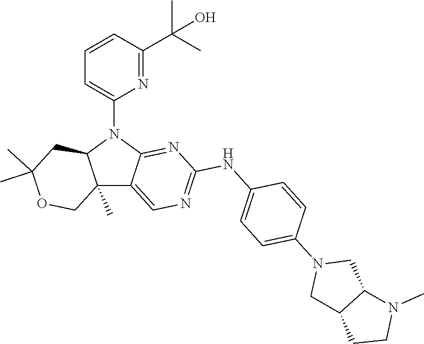 | 437, 438 | 1H NMR (300 MHz, DMSO-d6) δ 8.96 (s, 1H), 8.49 (d, J = 8.4 Hz, 1H), 7.94 (s, 1H), 7.74 (t, J = 7.8 Hz, 1H), 7.65-7.39 (m, 2H), 7.27 (d, J = 7.5 Hz, 1H), 6.57 (d, J = 8.7 Hz, 2H), 5.16 (s, 1H), 4.75-4.40 (m, 1H), 3.95 -3.55 (m, 2H), 3.32-3.18 (m, 2H), 3.16-2.99 (m, 2H), 2.99-2.75 (m, 3H), 2.32-2.18 (m, 5H), 2.15-1.96 (m, 1H), 1.64-1.50 (m, 2H), 1.45 (s, 6H), 1.28 (s, 3H), 1.23 (s, 3H), 1.04 (s, 3H). 1H NMR (300 MHz, DMSO-d6) δ 8.96 (s, 1H), 8.49 (d, J = 8.4 Hz, 1H), 7.94 (s, 1H), 7.74 (t, J = 7.8 Hz, 1H), 7.65-7.39 (m, 2H), 7.27 (d, J = 7.5 Hz, 1H), 6.57 (d, J = 8.7 Hz, 2H), 5.16 (s, 1H), 4.75-4.40 (m, 1H), 3.95 -3.55 (m, 2H), 3.32-3.18 (m, 2H), 3.16-2.99 (m, 2H), 2.99-2.75 (m, 3H), 2.32-2.18 (m, 5H), 2.15-1.96 (m, 1H), 1.64-1.50 (m, 2H), 1.45 (s, 6H), 1.28 (s, 3H), 1.23 (s, 3H), 1.04 (s, 3H). | LCMS (ES, m/z): 570 [M + H]+. Rt 0.649 min. LCMS (ES, m/z): 570 [M + H]+. Rt 0.651 min. |
| 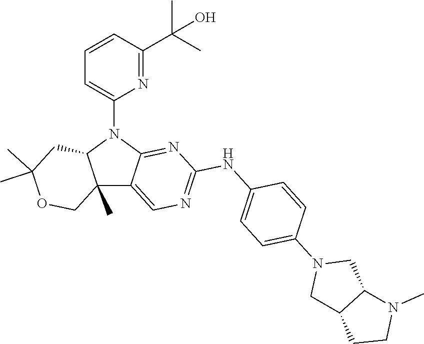 | | | |
| 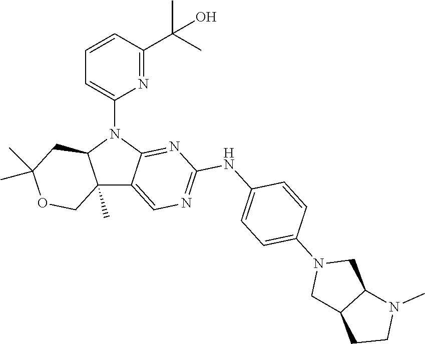 | 439, 440 | 1H NMR (300 MHz, DMSO-d6) δ 8.96 (s, 1H), 8.49 (d, J = 8.4 Hz, 1H), 7.94 (s, 1H), 7.79-7.68 (m, 1H), 7.47 (d, J = 8.7 Hz, 2H), 7.27 (d, J = 7.5 Hz, 1H), 6.57 (d, J = 9.0 Hz, 2H), 5.16 (s, 1H), 4.66-4.56 (m, 1H), 3.81-3.61 (m, 2H), 3.33-3.18 (m, 2H), 3.14-3.02 (m, 2H), 3.01-2.92 (m, 1H), 2.91-2.78 (m, 2H), 2.32-2.18 (m, 5H), 2.11-1.98 (m, 1H), 1.61-1.50 (m, 2H), 1.45 (s, 6H), 1.28 (s, 3H), 1.23 (s, 3H), 1.04 (s, 3H). 1H NMR (300 MHz, DMSO-d6) δ 8.96 (s, 1H), 8.49 (d, J = 8.4 Hz, 1H), 7.94 (s, 1H), 7.79-7.68 (m, 1H), 7.47 (d, J = 8.7 Hz, 2H), 7.27 (d, J = 7.5 Hz, 1H), 6.57 (d, J = 9.0 Hz, 2H), 5.16 (s, 1H), 4.66-4.55 (m, 1H), 3.81-3.61 (m, 2H), 3.32-3.18 (m, 2H), 3.14-3.02 (m, 2H), 3.01-2.90 (m, 1H), 2.910-2.78 (m, 2H), 2.32-2.18 (m, 5H), 2.11-1.96 (m, 1H), 1.68-1.50 (m, 2H), 1.45 (s, 6H), 1.28 (s, 3H), 1.23 | LCMS (ES, m/z): 570 [M + H]+, Rt 0.648 min. LCMS (ES, m/z): 570 [M + H]+, Rt 0.650 min. |
| | | (s, 3H), 1.04 (s, 3H). | |
| 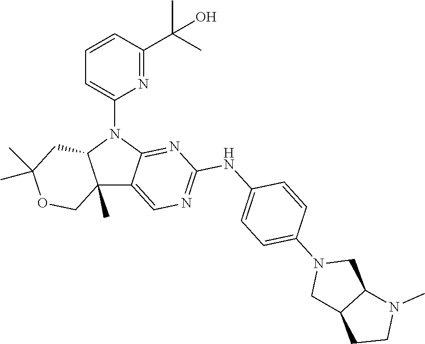 | | | |
| 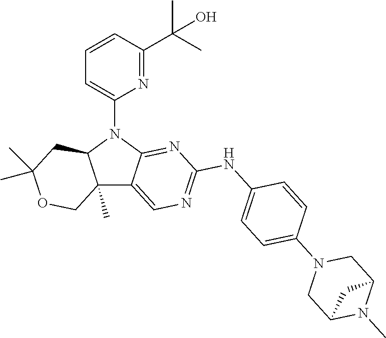 | 441, 442 | 1H NMR (300 MHz, DMSO-d6) δ 8.99 (s, 1H), 8.50 (d, J = 8.4 Hz, 1H), 7.94 (s, 1H), 7.74 (t, J = 8.1 Hz, 1H), 7.55 (d, J = 8.7 Hz, 2H), 7.27 (d, J = 7.5 Hz, 1H), 6.71 (d, J = 9.0 Hz, 2H), 5.17 (s, 1H), 4.66- 4.56 (m, 1H), 3.84-3.62 (m, 3H), 3.51- 3.45 (m, 2H), 3.42-3.37 (m, 3H), 2.30- 2.18 (m, 2H), 2.14-2.08 (m, 3H), 1.73- 1.51 (m, 2H), 1.45 (s, 6H), 1.29 (s, 3H), 1.23 (s, 3H), 1.04 (s, 3H). 1H NMR (300 MHz, DMSO-d6) δ 8.96 (s, 1H), 8.50 (d, J = 8.4 Hz, 1H), 7.94 (s, 1H), 7.80-7.68 (m, 1H), 7.57-7.48 (m, 2H), 7.27 (d, J = 7.5 Hz, 1H), 6.74-6.64 (m, 2H), 5.16 (s, 1H), 4.66-4.55 (m, 1H), 3.81-3.62 (m, 2H), 3.62-3.55 (m, 2H), 3.48-3.38 (m, 2H), 3.30-3.23 (m, 2H), 2.47-2.38 (m, 1H), 2.33-2.18 (m, 1H), 2.00 (s, 3H), 1.64-1.50 (m, 2H), 1.45 (s, 6H), 1.29 (s, 3H), 1.23 (s, 3H), 1.04 (s, 3H). | LCMS (ES, m/z): 556 [M + H]+; RT: 0.635 min. LCMS (ES, m/z): 556 [M + H]+; RT: 0.625 min. |
| 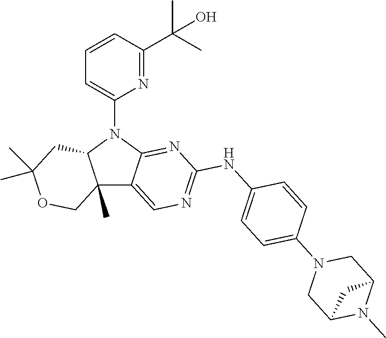 | | | |
| 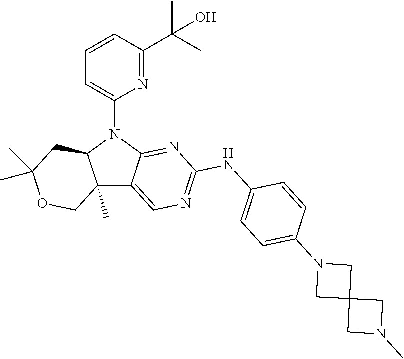 | 443, 444 | 1H NMR (300 MHz, DMSO-d6) δ 8.98 (s, 1H), 8.47 (d, J = 8.4 Hz, 1H), 7.93 (s, 1H), 7.73 (t, J = 8.4 Hz, 1H), 7.52 (d, J = 8.4 Hz, 2H), 7.30 (d, J = 7.5 Hz, 1H), 6.39 (d, J = 8.7 Hz, 2H), 5.17 (s, 1H), 4.63- 4.58 (m, 1H), 3.79-3.58 (m, 6H), 3.33- 3.24 (m, 4H), 2.24-2.18 (m, 4H), 1.57- 1.46 (m, 1H), 1.45 (s, 6H), 1.27 (s, 3H), 1.22 (s, 3H), 1.03 (s, 3H). 1H NMR (300 MHz, DMSO-d6) δ 8.98 (s, 1H), 8.46 (d, J = 8.1 Hz, 1H), 7.94 (s, 1H), 7.73 (t, J = 8.1 Hz, 1H), 7.47 (d, J = 8.4 Hz, 2H), 7.28 (d, J = 7.5 Hz, 1H), 6.39 (d, J = 8.7 Hz , 2H), 5.17 (s, 1H), 4.63- 4.58 (m, 1H), 3.79-3.58 (m, 6H), 3.33- 3.24 (m, 4H), 2.27-2.20 (m, 4H), 1.61- 1.53 (m, 1H), 1.45 (s, 6H), 1.28 (s, 3H), 1.23 (s, 3H), 1.03 (s, 3H). | LCMS (ES, m/z): 556 [M + H]+, Rt 1.289 min. LCMS (ES, m/z): 556 [M + H]+, Rt 1.289 min. |
| 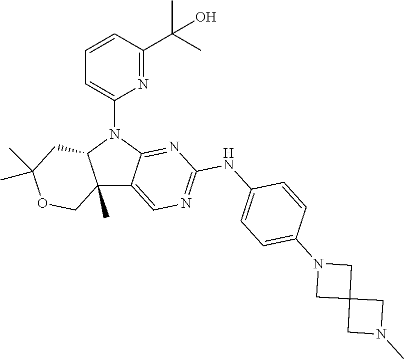 | | | |
| 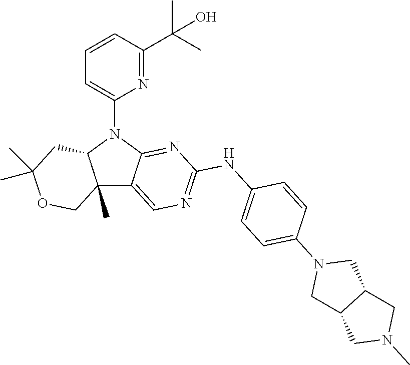 | 445, 446 | 1H NMR (400 MHz, DMSO-d6) δ 8.97 (s, 1H), 8.49 (d, J = 8.4 Hz, 1H), 7.93 (s, 1H), 7.73 (t, J = 8.0 Hz, 1H), 7.51 (d, J = 8.8 Hz, 2H), 7.28 (d, J = 7.6 Hz, 1H), 6.29-6.60 (m, 2H), 5.16 (s, 1H), 4.62- 4.58 (m, 1H), 3.78-3.67 (m, 2H), 3.32- 3.27 (m, 2H), 3.08-3.01 (m, 2H), 2.87- 2.82 (m, 2H), 2.60-2.58 (m, 2H), 2.38- 2.32 (m, 2H), 2.26-2.22 (m, 4H), 1.59- 1.51 (m, 1H), 1.47 (s, 6H), 1.28 (s, 3H), 1.23 (s, 3H), 1.04 (s, 3H). 1H NMR (400 MHz, DMSO-d6) δ 8.97 (s, 1H), 8.49 (d, J = 8.4 Hz, 1H), 7.93 (s, 1H), 7.73 (t, J = 8.0 Hz, 1H), 7.49 (d, J = 8.8 Hz, 2H), 7.28 (d, J = 7.6 Hz, 1H), 6.29-6.60 (m, 2H), 5.15 (s, 1H), 4.62- 4.58 (m, 1H), 3.81-3.62 (m, 2H), 3.32- 3.27 (m, 2H), 3.09-3.01 (m, 2H), 2.88- 2.82 (m, 2H), 2.60-2.58 (m, 2H), 2.38- 2.32 (m, 2H), 2.26-2.22 (m, 4H), 1.59- 1.45 (m, 7H), 1.28 (s, 3H), 1.23 (s, 3H), 1.04 (s, 3H). | LCMS (ES, m/z): 570 [M + H]+. Rt 0.633 min. LCMS (ES, m/z): 570 [M + H]+. Rt 0.636 min. |
| 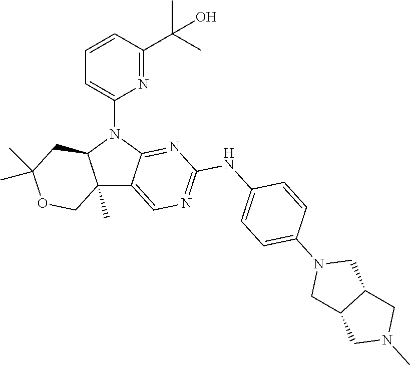 | | | |
| 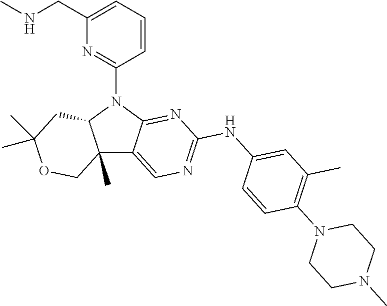 | 447, 448 | 1H NMR (300 MHz, DMSO-d6) δ 9.15 (s, 1H), 8.48 (d, J = 8.1 Hz, 1H), 8.01 (s, 1H), 7.74 (t, J = 7.5 Hz, 1H), 7.59 (d, J = 2.4 Hz, 1H), 7.40 (d, J = 8.4 Hz, 1H), 7.04 (d, J = 7.5 Hz, 1H), 6.97 (d, J = 8.7 Hz, 1H), 4.72-4.67 (m, 1H), 3.80-3.63 (m, 4H), 2.88-2.79 (m, 4H), 2.35-2.30 (m, 4H), 2.34 (s, 3H), 2.30-2.15 (m, 7H), 1.69-1.58 (m, 1H), 1.29 (s, 3H), 1.23 (s, 3H), 1.02 (s, 3H). 1H NMR (300 MHz, DMSO-d6) δ 9.15 (s, 1H), 8.48 (d, J = 8.4 Hz, 1H), 8.01 (s, 1H), 7.72 (t, J = 7.8 Hz, 1H), 7.59 (d, J = 2.4 Hz, 1H), 7.40 (d, J = 8.7 Hz, 1H), 7.04 (d, J = 7.5 Hz, 1H), 6.97 (d, J = 8.4 Hz, 1H), 4.72-4.67 (m, 1H), 3.80-3.63 (m, 4H), 2.88-2.79 (m, 4H), 2.41-2.34 (m, 4H), 2.34 (s, 3H), 2.30-2.15 (m, 7H), 1.69-1.58 (m, 1H), 1.29 (s, 3H), 1.23 (s, | LCMS (ES, m/z): 543 [M + H]+. Rt 0.525 min. LCMS (ES, m/z): 543 [M + H]+. Rt 0.532 min. |
| | | 3H), 1.02 (s, 3H). | |
| 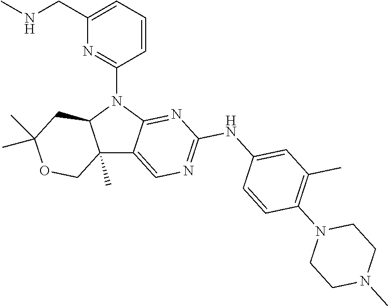 | | | |
| 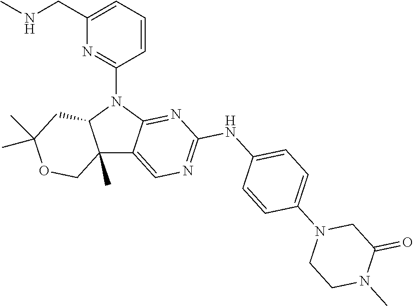 [SMILES: CNCc1cccc(N2c3nc(Nc4ccc(N5CCN(C)C(=O)C5)cc4)ncc3[C@]3(C)COC(C)(C)C[C@H]23)n1] | 449, 450 | 1H NMR (300 MHz, DMSO-d6) δ 9.15 (s, 1H), 8.52 (d, J = 8.4 Hz, 1H), 8.01 (s, 1H), 7.80 (t, J = 7.5 Hz, 1H), 7.59 (d, J = 8.7 Hz, 2H), 7.06 (d, J = 7.2 Hz, 1H), 6.99-6.88 (m, 2H), 4.78-4.68 (m, 1H), 3.86 (s, 2H), 3.81-3.62 (m, 4H), 3.47- 3.40 (m, 4H), 2.90 (s, 3H), 2.43 (s, 3H), 2.26-2.14 (m, 1H), 1.69-1.55 (m, 1H), 1.28 (s, 3H), 1.23 (s, 3H), 1.01 (s, 3H). 1H NMR (300 MHz, DMSO-d6) δ 9.15 (s, 1H), 8.54 (d, J = 8.4 Hz, 1H), 8.01 (s, 1H), 7.81 (t, J = 7.5 Hz, 1H), 7.64-7.53 (m, 2H), 7.07 (d, J = 7.2 Hz, 1H), 6.99- 6.88 (m, 2H), 4.80-4.70 (m, 1H), 3.91 (s, 2H), 3.82-3.62 (m, 4H), 3.48-3.37 (m, 4H), 2.90 (s, 3H), 2.47 (s, 3H), 2.27-2.15 (m, 1H), 1.68-1.55 (m, 1H), 1.27 (s, 3H), 1.23 (s, 3H), 1.01 (s, 3H). | LCMS (ES, m/z): 543 [M + H]+; RT: 0.586 min. LCMS (ES, m/z): 543 [M + H]+; RT: 0.580 min. |
| 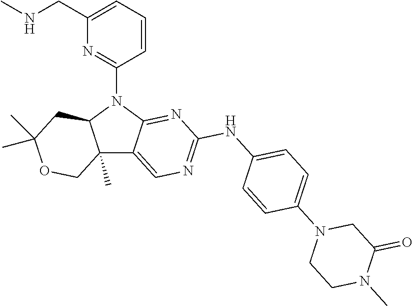 [SMILES: CNCc1cccc(N2c3nc(Nc4ccc(N5CCN(C)C(=O)C5)cc4)ncc3[C@@]3(C)COC(C)(C)C[C@@H]23)n1] | | | |
| 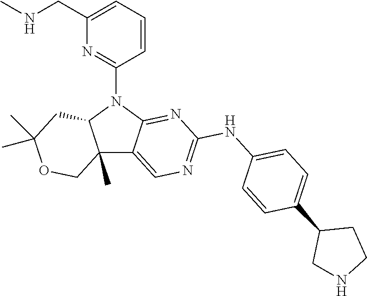 | 451, 452 | 1H NMR (300 MHz, DMSO-d6) δ 9.35- 9.26 (m, 1H), 8.50 (d, J = 8.1 Hz, 1H), 8.03 (s, 1H), 7.79-7.64 (m, 3H), 7.20 (t, J = 8.0 Hz, 2H), 7.06 (d, J = 7.2 Hz, 1H), 4.72-4.67 (m, 1H), 3.82-3.61 (m, 5H), 3.50-3.40 (m, 2H), 3.35-3.30 (m, 1H), 3.22-3.15 (m, 1H), 3.09-2.93 (m, 1H), 2.79-2.57 (m, 1H), 2.34 (s, 3H), 2.23- 2.12 (m, 1H), 1.68-1.61 (m, 2H), 1.29 (s, 3H), 1.23 (s, 3H), 1.02 (s, 3H). 1H NMR (300 MHz, DMSO-d6) δ 9.38- 9.23 (m, 1H), 8.50 (d, J = 8.1 Hz, 1H), 8.03 (s, 1H), 7.78-7.63 (m, 3H), 7.20 (t, J = 9.0 Hz, 2H), 7.06 (d, J = 7.2 Hz, 1H), 4.72-4.67 (m, 1H), 3.80-3.64 (m, 5H), 3.52-3.46 (m, 1H), 3.22-3.16 (m, 2H), 3.07-2.93 (m, 2H), 2.78-2.55 (m, 1H), 2.34 (s, 3H), 2.30-2.03 (m, 1H), 1.68- | LCMS (ES, m/z): 500 [M + H]+, Rt 0.513 min. LCMS (ES, m/z): 500 [M + H]+, Rt 0.512 min. |
| | | 1.61 (m, 2H), 1.29 (s, 3H), 1.23 (s, 3H), | |
| | | 1.02 (s, 3H). | |
| 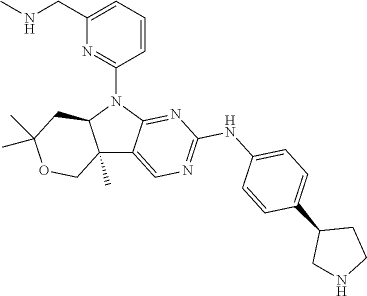 [SMILES: CNCc1cccc(N2c3nc(Nc4ccc([C@H]5CCNC5)cc4)ncc3[C@@]3(C)COC(C)(C)C[C@@H]23)n1] | | | |
| 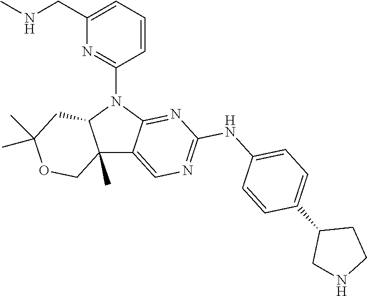 | 453, 454 | 1H NMR (300 MHz, DMSO-d6) δ 9.34- 9.24 (m, 1H), 8.50 (d, J = 8.4 Hz, 1H), 8.03 (s, 1H), 7.80-7.60 (m, 3H), 7.20 (t, J = 8.4 Hz, 2H), 7.06 (d, J = 7.5 Hz, 1H), 4.75-4.65 (m, 1H), 3.82-3.60 (m, 4H), 3.53-3.50 (m, 2H), 3.35-3.25 (m, 2H), 3.24-3.15 (m, 1H), 3.55-2.61 (m, 2H), 2.34 (s, 3H), 2.25-2.10 (m, 2H), 1.71- 1.57 (m, 1H), 1.29 (s, 3H), 1.22 (s, 3H), 1.01 (s, 3H). 1H NMR (300 MHz, DMSO-d6) δ 9.34- 9.24 (m, 1H), 8.50 (d, J = 8.4 Hz, 1H), 8.03 (s, 1H), 7.80-7.60 (m, 3H), 7.25- 7.16 (m, 2H), 7.06 (d, J = 7.2 Hz, 1H), 4.75-4.64 (m, 1H), 3.82-3.60 (m, 5H), 3.55-3.40 (m, 1H), 3.36-3.10 (m, 3H), 3.55-2.61 (m, 2H), 2.34 (s, 3H), 2.25- 2.10 (m, 2H), 1.71-1.57 (m, 1H), 1.29 (s, | LCMS (ES, m/z): 500 [M + H]+, Rt 0.508 min. LCMS (ES, m/z): 500 [M + H]+, Rt 0.958 min. |
| | | 3H), 1.22 (s, 3H), 1.01 (s, 3H). | |
| 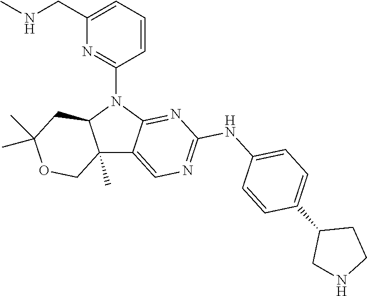 [SMILES: CNCc1cccc(N2c3nc(Nc4ccc([C@@H]5CCNC5)cc4)ncc3[C@@]3(C)COC(C)(C)C[C@@H]23)n1] | | | |
| 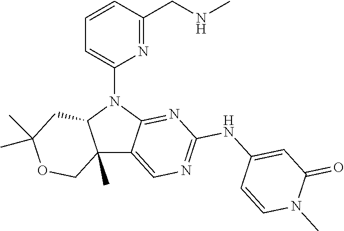 [SMILES: CNCc1cccc(N2c3nc(Nc4ccn(C)c(=O)c4)ncc3[C@]3(C)COC(C)(C)C[C@H]23)n1] | 455, 456 | 1H NMR (300 MHz, DMSO-d6) δ 9.59 (s, 1H), 8.44 (d, J = 8.4 Hz, 1H), 8.13 (s, 1H), 7.77 (t, J = 8.0 Hz, 1H), 7.51 (d, J = 7.5 Hz, 1H), 7.13-7.05 (m, 2H), 6.54- 6.45 (m, 1H), 4.78-4.67 (m, 1H), 3.95 - 3.62 (m, 4H), 3.28-3.25 (m, 3H), 2.34 (s, 3H), 2.26-2.13 (m, 1H), 1.74-1.60 (m, 1H), 1.30 (s, 3H), 1.22 (s, 3H), 1.00 (s, 3H). 1H NMR (300 MHz, DMSO-d6) δ 9.59 (s, 1H), 8.44 (d, J = 8.4 Hz, 1H), 8.13 (s, 1H), 7.77 (t, J = 7.9 Hz, 1H), 7.51 (d, J = 7.5 Hz, 1H), 7.13-7.05 (m, 2H), 6.54- 6.45 (m, 1H), 4.78-4.67 (m, 1H), 3.95 - | LCMS (ES, m/z): 462 [M + H]+. Rt 0.500 min. LCMS (ES, m/z): 462 [M + H]+. Rt 0.692 min. |
| | | 3.62 (m, 4H), 3.28-3.25 (m, 3H), 2.34 (s, | |
| | | 3H), 2.26-2.13 (m, 1H), 1.74-1.60 (m, | |
| | | 1H), 1.30 (s, 3H), 1.22 (s, 3H), 1.00 (s, | |
| | | 3H). | |
| 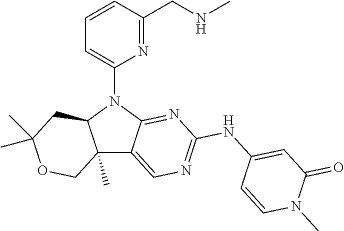 [SMILES: CNCc1cccc(N2c3nc(Nc4ccn(C)c(=O)c4)ncc3[C@@]3(C)COC(C)(C)C[C@@H]23)n1] | | | |
| 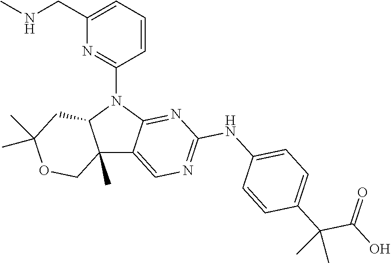 [SMILES: CNCc1cccc(N2c3nc(Nc4ccc(C(C)(C)C(=O)O)cc4)ncc3[C@]3(C)COC(C)(C)C[C@H]23)n1] | 457, 458 | 1H NMR (300 MHz, DMSO-d6) δ 9.29 (s, 1H), 8.50 (d, J = 8.4 Hz, 1H), 8.02 (s, 1H), 7.75-7.70 (m, 1H), 7.67-7.64 (m, 2H), 7.26-7.23 (m, 2H), 7.06-7.03 (m, 1H), 4.71-4.67 (m, 1H), 3.79-3.75 (m, 1H), 3.72-3.63 (m, 2H), 3.52-3.42 (m, 1H), 2.34 (s, 3H), 2.22-2.16 (m, 1H), 1.66-1.59 (m, 1H), 1.46 (s, 6H), 1.27 (s, 3H), 1.23 (s, 3H), 1.02 (s, 3H). 1H NMR (300 MHz, DMSO-d6) δ 9.29 (s, 1H), 8.50 (d, J = 8.4 Hz, 1H), 8.02 (s, 1H), 7.75-7.70 (m, 1H), 7.66-7.64 (m, 2H), 7.26-7.24 (m, 2H), 7.05-7.03 (m, 1H), 4.71-4.66 (m, 1H), 3.79-3.75 (m, 1H), 3.71-3.67 (m, 2H), 3.63-3.60 (m, 1H), 2.34 (s, 3H), 2.21-2.16 (m, 1H), | LCMS (ES, m/z): 517 M+H]+. Rt 0.610 min. LCMS (ES, m/z): 517 [M + H]+. Rt 0.616 min. |
| | | 1.66-1.63 (m, 1H), 1.46 (s, 6H), 1.27 | |
| | | (s, 3H), 1.21 (s, 3H), 1.00 (s, 3H). | |
| 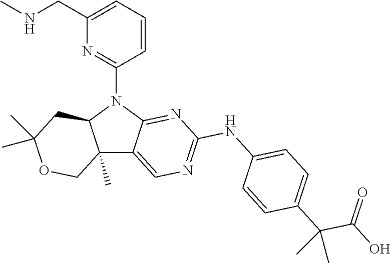 [SMILES: CNCc1cccc(N2c3nc(Nc4ccc(C(C)(C)C(=O)O)cc4)ncc3[C@@]3(C)COC(C)(C)C[C@@H]23)n1] | | | |
| 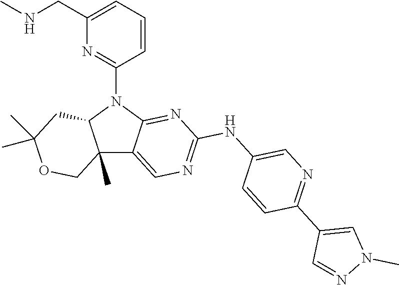 | 459, 460 | 1H NMR NMR (400 MHz, DMSO-d6) δ 9.53 (s, 1H), 8.79 (d, J = 2.4 Hz, 1H), 8.50 (d, J = 7.6 Hz, 1H), 8.20 (d, J = 8.4 Hz, 1H), 8.17 (s, 1H), 8.08 (s, 1H), 7.92 (s, 1H), 7.80 (t, J = 8.0 Hz, 1H), 7.58 (d, J = 8.8 Hz, 1H), 7.09 (d, J = 7.2 Hz, 1H), 4.77-4.72 (m, 1H), 3.97-3.76 (m, 6H), 3.72-3.65 (m, 1H), 2.43 (s, 3H), 2.23- 2.18 (m, 1H), 1.69-1.64 (m, 1H), 1.30 (s, 3H), 1.23 (s, 3H), 1.02 (s, 3H). 1H NMR NMR (300 MHz, DMSO-d6) δ 9.50 (s, 1H), 8.78 (d, J = 2.4 Hz, 1H), 8.47 (d, J = 8.1 Hz, 1H), 8.20 (d, J = 8.7 Hz, 1H), 8.16 (s, 1H), 8.06 (s, 1H), 7.91 (s, 1H), 7.78 (t, J = 7.8 Hz, 1H), 7.56 (d, J = 8.4 Hz, 1H), 7.07 (d, J = 7.1 Hz, 1H), 4.77-4.72 (m, 1H), 3.97-3.76 (m, 6H), | LCMS (ES, m/z): 512 [M + H]+. Rt 0.554 min. LCMS (ES, m/z): 512 [M + H]+. Rt 0.553 min. |
| | | 3.72-3.65 (m, 1H), 2.38 (s, 3H), 2.23- | |
| | | 2.18 (m, 1H), 1.69-1.64 (m, 1H), 1.29 (s, | |
| | | 3H), 1.22 (s, 3H), 1.01 (s, 3H). | |
| 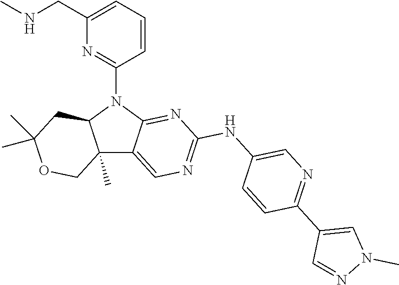 | | | |
| 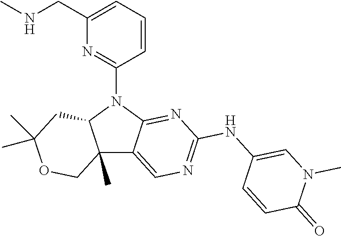 | 461, 462 | 1H NMR (400 MHz, DMSO-d6) δ 8.94 (s, 1H), 8.39-8.34 (m, 1H), 8.07 (s, 1H), 7.98 (s, 1H), 7.75 (d, J = 7.6 Hz, 1H), 7.58-7.55 (m, 1H), 7.05 (d, J = 7.6 Hz, 1H), 6.42 (d, J = 9.6 Hz, 1H), 4.69-4.66 (m, 1H), 3.76-3.63 (m, 4H), 3.43 (s, 3H), 2.34 (s, 3H), 2.20-2.16 (m, 1H), 1.64- 1.59 (m, 1H), 1.27 (s, 3H), 1.21 (s, 3H), 1.01 (s, 3H). 1H NMR (400 MHz, DMSO-d6) δ 8.94 (s, 1H), 8.40-8.35 (m, 1H), 8.07 (s, 1H), 7.98 (s, 1H), 7.75 (d, J = 8.0 Hz, 1H), 7.58-7.55 (m, 1H), 7.05 (d, J = 7.6 Hz, 1H), 6.42 (d, J = 9.2 Hz, 1H), 4.69-4.66 (m, 1H), 3.76-3.63 (m, 4H), 3.43 (s, 3H), | LCMS (ES, m/z): 462 [M + H]+. Rt 0.509 min. LCMS (ES, m/z): 462 [M + H]+. Rt 0.511 min. |
| | | 2.33 (s, 3H), 2.20-2.16 (m, 1H), 1.64- | |
| | | 1.59 (m, 1H), 1.26 (s, 3H), 1.21 (s, 3H), | |
| | | 1.01 (s, 3H). | |
| 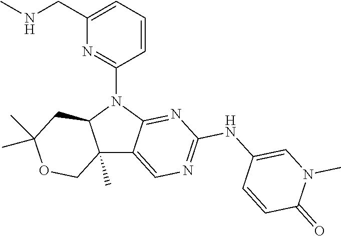 | | | |
| 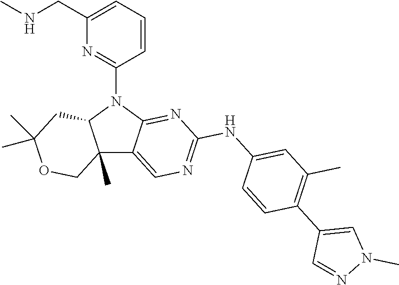 | 463, 464 | 1H NMR (300 MHz, DMSO-d6) δ 9.33 (s, 1H), 8.50 (d, J = 8.4 Hz, 1H), 8.05 (s, 1H), 7.88 (s, 1H), 7.77 (t, J = 7.8 Hz, 1H), 7.72-7.66 (m, 1H), 7.63 (s, 1H), 7.56- 7.47 (m, 1H), 7.27 (d, J = 8.4 Hz, 1H), 7.06 (d, J = 7.5 Hz, 1H), 4.76-4.65 (m, 1H), 3.89 (s, 3H), 3.83-3.75 (m, 1H), 3.75-3.60 (m, 3H), 2.39-2.31 (m, 6H), 2.27-2.15 (m, 1H), 1.72-1.58 (m, 1H), 1.29 (s, 3H), 1.23 (s, 3H), 1.02 (s, 3H). 1H NMR (300 MHz, DMSO-d6) δ 9.33 (s, 1H), 8.50 (d, J = 8.4 Hz, 1H), 8.05 (s, 1H), 7.88 (s, 1H), 7.77 (t, J = 7.8 Hz, 1H), 7.72-7.66 (m, 1H), 7.63 (s, 1H), 7.56- 7.47 (m, 1H), 7.27 (d, J = 8.4 Hz, 1H), 7.06 (d, J = 7.5 Hz, 1H), 4.76-4.65 (m, 1H), 3.89 (s, 3H), 3.83-3.60(m, 4H), | LCMS (ES, m/z): 525 [M + H]+. Rt 0.636 min. LCMS (ES, m/z): 525 [M + H]+. Rt 0.639 min. |
| | | 2.39-2.31 (m, 6H), 2.27-2.15 (m, 1H), | |
| | | 1.72-1.58 (m, 1H), 1.29 (s, 3H), 1.23 (s, | |
| | | 3H), 1.02 (s, 3H). | |
| 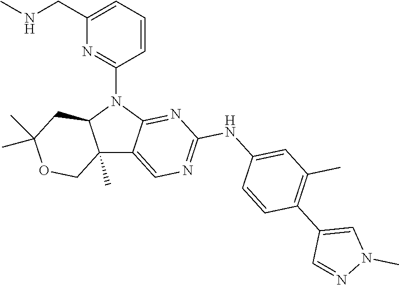 | | | |
| 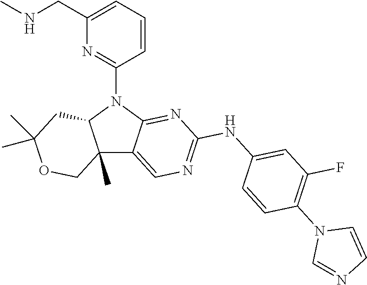 [SMILES: CNCc1cccc(N2c3nc(Nc4ccc(-n5ccnc5)c(F)c4)ncc3[C@]3(C)COC(C)(C)C[C@H]23)n1] | 465, 466 | 1H NMR (300 MHz, DMSO-d6) δ 9.80 (s, 1H), 8.46 (d, J = 8.4 Hz, 1H), 8.13-8.08 (m, 2H), 7.95 (s, 1H), 7.82-7.77 (m, 1H), 7.60-7.48 (m, 3H), 7.10-7.07 (m, 2H), 4.75-4.70 (m, 1H), 3.80-3.64 (m, 4H), 2.34 (s, 3H), 2.22-2.16 (m, 1H), 1.71- 1.64 (m, 1H), 1.30 (s, 3H), 1.21 (s, 3H), 1.00 (s, 3H). 1H NMR (400 MHz, DMSO-d6) δ 9.80 (s, 1H), 8.46 (d, J = 8.4 Hz, 1H), 8.13-8.08 (m, 2H), 7.95 (s, 1H), 7.82-7.77 (m, 1H), 7.60-7.48 (m, 3H), 7.10-7.07 (m, 2H), 4.74-4.70 (m, 1H), 3.80-3.64 (m, 4H), 2.33 (s, 3H), 2.22-2.16 (m, 1H), 1.71- 1.64 (m, 1H), 1.30 (s, 3H), 1.21 (s, 3H), 1.15 (s, 3H). | LCMS (ES, m/z): 515 [M + H]+. Rt 0.593 min LCMS (ES, m/z): 515 [M + H]+. Rt 0.532 min. |
| 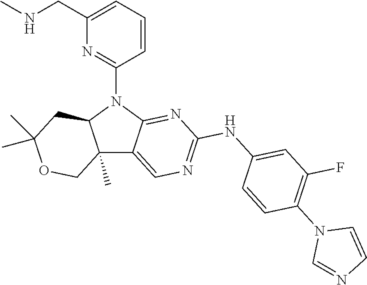 [SMILES: CNCc1cccc(N2c3nc(Nc4ccc(-n5ccnc5)c(F)c4)ncc3[C@@]3(C)COC(C)(C)C[C@@H]23)n1] | | | |
| 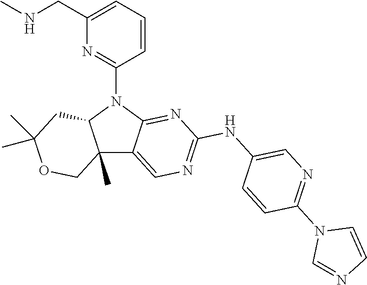 [SMILES: CNCc1cccc(N2c3nc(Nc4ccc(-n5ccnc5)nc4)ncc3[C@]3(C)COC(C)(C)C[C@H]23)n1] | 467, 468 | 1H NMR NMR (400 MHz, DMSO-d6) δ 9.70 (s, 1H), 8.11 (d, J = 2.8 Hz, 1H), 8.47-8.40 (m, 3H), 8.10 (s, 1H), 7.90- 7.89 (m, 1H), 7.83-7.75 (m, 2H), 7.11- 7.08 (m, 2H), 4.75-4.72 (m, 1H), 3.81- 3.66 (m, 4H), 2.37 (s, 3H), 2.23-2.18 (m, 1H), 1.69-1.64 (m, 1H), 1.30 (s, 3H), 1.23 (s, 3H), 1.01 (s, 3H). 1H NMR (400 MHz, DMSO-d6) δ 9.70 (s, 1H), 8.82 (d, J = 2.8 Hz, 1H), 8.47-8.40 (m, 3H), 8.10 (s, 1H), 7.89 (s, 1H), 7.83- 7.75 (m, 2H), 7.12-7.08 (m, 2H), 4.75- 4.72 (m, 1H), 3.81-3.66 (m, 4H), 2.36 (s, 3H), 2.23-2.18 (m, 1H), 1.69-1.64 (m, 1H), 1.30 (s, 3H), 1.23 (s, 3H), 1.01 (s, 3H). | LCMS (ES, m/z): 498 [M + H]+. Rt 0.519 min. LCMS (ES, m/z): 498 [M + H]+. Rt 0.518 min. |
| 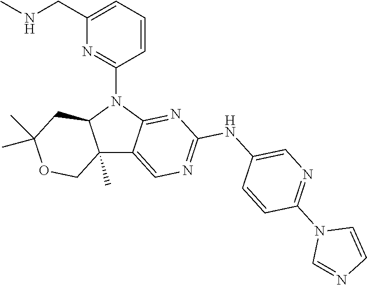 [SMILES: CNCc1cccc(N2c3nc(Nc4ccc(-n5ccnc5)nc4)ncc3[C@@]3(C)COC(C)(C)C[C@@H]23)n1] | | | |
| 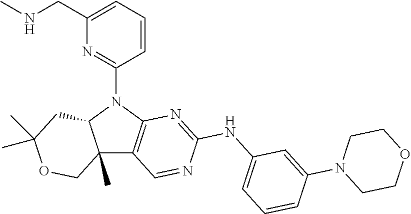 [SMILES: CNCc1cccc(N2c3nc(Nc4cccc(N5CCOCC5)c4)ncc3[C@]3(C)COC(C)(C)C[C@H]23)n1] | 469, 470 | 1H NMR (400 MHz, DMSO-d6) δ 9.17 (s, 1H), 8.48 (d, J = 8.4 Hz, 1H), 8.03 (s, 1H), 7.72 (t, J = 8.0 Hz, 1H), 7.41-7.35 (m, 1H), 7.25-7.18 (m, 1H), 7.12 (t, J = 8.0 Hz, 1H), 7.05 (d, J = 7.6 Hz, 1H), 6.58-6.51 (m, 1H), 4.82-4.55 (m, 1H), 3.80-3.61 (m, 8H), 3.10-3.01 (m, 4H), 2.34 (s, 3H), 2.24-2.15 (m, 1H), 1.78- 1.51 (m, 1H), 1.28 (s, 3H), 1.22 (s, 3H), 1.01 (s, 3H). 1H NMR (400 MHz, DMSO-d6) δ 9.17 (s, 1H), 8.48 (d, J = 8.4 Hz, 1H), 8.03 (s, 1H), 7.72 (t, J = 8.0 Hz, 1H), 7.41-7.35 | LCMS (ES, m/z): 516 [M + H]+. Rt 0.575 min. LCMS (ES, m/z): 516 [M + H]+. Rt 0.578 min. |
| | | (m, 1H), 7.25-7.18 (m, 1H), 7.12 (t, J = | |
| | | 8.0 Hz, 1H), 7.05 (d, J = 7.6 Hz, 1H), | |
| | | 6.58-6.51 (m, 1H), 4.82-4.55 (m, 1H), | |
| | | 3.90-3.61 (m, 8H), 3.10-3.01 (m, 4H), | |
| | | 2.34 (s, 3H), 2.24-2.15 (m, 1H), 1.78- | |
| | | 1.51 (m, 1H), 1.28 (s, 3H), 1.22 (s, 3H), | |
| | | 1.01 (s, 3H). | |
| 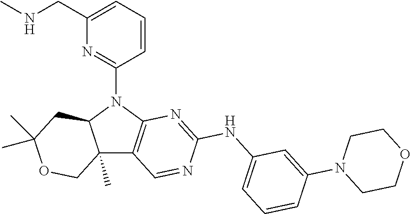 [SMILES: CNCc1cccc(N2c3nc(Nc4cccc(N5CCOCC5)c4)ncc3[C@@]3(C)COC(C)(C)C[C@@H]23)n1] | | | |
| 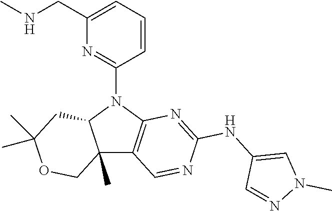 | 471 472 | 1H NMR (400 MHz, DMSO-d6) δ 9.15 (s, 1H), 8.48 (s, 1H), 8.00 (s, 1H), 7.87-7.75 (m, 2H), 7.47 (s, 1H), 7.06 (d, J = 7.5 Hz, 1H), 4.74-4.66 (m, 1H), 3.84-3.79 (m, 5H), 3.79-3.72 (m, 1H), 3.69-3.61 (m, 1H), 2.41 (s, 3H), 2.24-2.14 (m, 1H), 1.67-1.57 (m, 1H), 1.27 (s, 3H), 1.22 (s, 3H), 1.01 (s, 3H). 1H NMR (400 MHz, DMSO-d6) δ 9.14 (s, 1H), 8.47-8.42 (m, 1H), 7.98 (s, 1H), 7.88-7.73 (m, 2H), 7.46 (s, 1H), 7.05 (d, J = 7.4 Hz, 1H), 4.71-4.63 (m, 1H), 3.85- 3.61 (m, 7H), 2.34 (s, 3H), 2.26-2.14 (m, | LCMS (ES, m/z): 435 [M + H]+; RT: 0.923 min. LCMS (ES, m/z): 435 [M + H]+; RT: 0.501 min. |
| | | 1H), 1.68-1.58 (m, 1H), 1.27 (s, 3H), | |
| | | 1.22 (s, 3H), 1.01 (s, 3H). | |
| 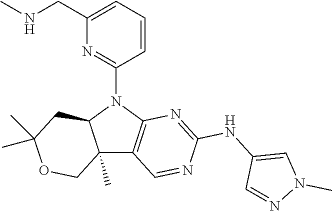 | | | |
| 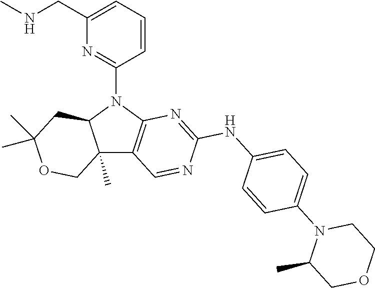 [SMILES: CNCc1cccc(N2c3nc(Nc4ccc(N5CCOC[C@H]5C)cc4)ncc3[C@@]3(C)COC(C)(C)C[C@@H]23)n1] | 473, 474 | 1H NMR (400 MHz, DMSO-d6) δ 9.13 (s, 1H), 8.62 (d, J = 8.4 Hz, 1H), 8.04 (s, 1H), 7.84 (t, J = 8.0 Hz, 1H), 7.55 (d, J = 8.8 Hz, 2H), 7.08 (d, J = 7.6 Hz, 1H), 6.89 (d, J = 8.8 Hz, 2H), 4.98-4.79 (m, 1H), 4.33-4.20 (m, 2H), 3.90-3.82 (m, 1H), 3.82-3.74 (m, 2H), 3.74-3.55 (m, 4H), 3.09-2.92 (m, 2H), 2.70 (s, 3H), 2.23- 2.13 (m, 1H), 1.85-1.60 (m, 1H), 1.27 (s, 3H), 1.23 (s, 3H), 1.01 (s, 3H), 0.93 (d, J = 6.4 Hz, 3H). 1H NMR (400 MHz, DMSO-d6) δ 9.13 (s, 1H), 8.62 (d, J = 8.4 Hz, 1H), 8.04 (s, 1H), 7.84 (t, J = 8.0 Hz, 1H), 7.55 (d, J = 8.8 Hz, 2H), 7.08 (d, J = 7.6 Hz, 1H), 6.89 (d, J = 8.8 Hz, 2H), 4.98-4.79 (m, 1H), 3.99-3.82 (m, 1H), 3.82-3.74 (m, 4H), 3.74-3.68 (m, 2H), 3.68-3.59 (m, 2H), | LCMS (ES, m/z): 530 [M + H]+. Rt 0.566 min. LCMS (ES, m/z): 530 [M + H]+. Rt 0.557 min. |
| | | 3.09-2.92 (m, 2H), 2.70 (s, 3H), 2.23- | |
| | | 2.13 (m, 1H), 1.85-1.60 (m, 1H), 1.27 (s, | |
| | | 3H), 1.23 (s, 3H), 1.01 (s, 3H), 0.93 (d, | |
| | | J = 6.4 Hz, 3H). | |
| 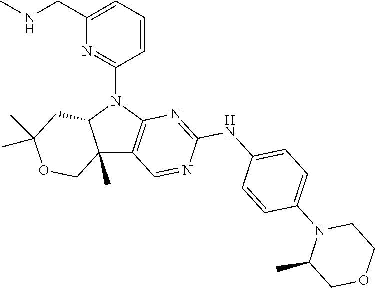 [SMILES: CNCc1cccc(N2c3nc(Nc4ccc(N5CCOC[C@H]5C)cc4)ncc3[C@]3(C)COC(C)(C)C[C@H]23)n1] | | | |
| 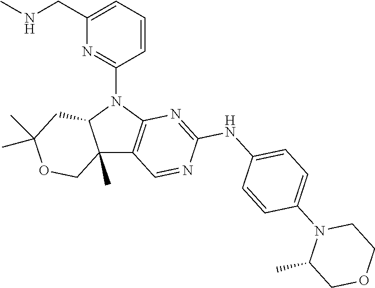 [SMILES: CNCc1cccc(N2c3nc(Nc4ccc(N5CCOC[C@@H]5C)cc4)ncc3[C@]3(C)COC(C)(C)C[C@H]23)n1] | 475, 476 | 1H NMR (300 MHz, DMSO-d6) δ 9.08 (s, 1H), 8.48 (d, J = 8.4 Hz, 1H), 7.98 (s, 1H), 7.72 (t, J = 7.8 Hz, 1H), 7.56 (d, J = 9.0 Hz, 2H), 7.03 (d, J = 7.2 Hz, 1H), 6.88 (d, J = 9.0 Hz, 2H), 4.68-4.65 (m, 1H), 3.87-3.83 (m, 1H), 3.77-3.55 (m, 8H), 3.03-2.96 (m, 2H), 2.34 (s, 3H), 2.20-2.15 (m, 1H), 1.62-1.56 (m, 1H), 1.26 (s, 3H), 1.21 (s, 3H), 1.00 (s, 3H), 0.92 (d, J = 6.3 Hz, 3H). 1H NMR (300 MHz, DMSO-d6) δ 9.08 (s, 1H), 8.48 (d, J = 8.4 Hz, 1H), 7.98 (s, 1H), 7.72 (t, J = 7.8 Hz, 1H), 7.56 (d, J = 9.0 Hz, 2H), 7.03 (d, J = 7.2 Hz, 1H), 6.88 (d, J = 9.0 Hz, 2H), 4.68-4.65 (m, 1H), 3.87-3.83 (m, 1H), 3.77-3.55 (m, 8H), 3.03-2.96 (m, 2H), 2.34 (s, 3H), 2.20-2.17 (m, 1H), 1.66-1.62 (m, 1H), | LCMS (ES, m/z): 530 [M + H]+, Rt 0.554 min. LCMS (ES, m/z): 530 [M + H]+, Rt 0.559 min. |
| | | 1.27 (s, 3H), 1.21 (s, 3H), 1.00 (s, 3H), | |
| | | 0.92 (d, J = 6.3 Hz, 3H). | |
| 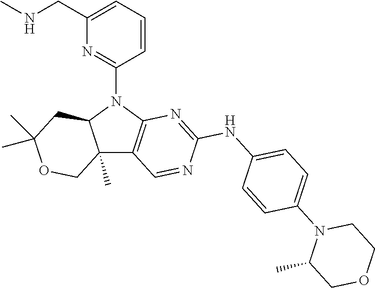 [SMILES: CNCc1cccc(N2c3nc(Nc4ccc(N5CCOC[C@@H]5C)cc4)ncc3[C@@]3(C)COC(C)(C)C[C@@H]23)n1] | | | |
| 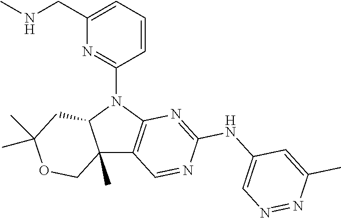 | 477, 478 | 1H NMR (400 MHz, DMSO-d6) δ 10.06 (s, 1H), 9.19 (d, J = 2.0 Hz, 1H), 8.38 (d, J = 8.4 Hz, 1H), 8.19 (s, 1H), 8.09 (s, 1H), 7.85-7.81 (m, 1H), 7.12 (d, J = 7.2 Hz, 1H), 4.77-4.74(m, 1H), 3.91-3.66 (m, 5H), 2.53 (s, 3H), 2.36 (s, 3H), 2.21-2.17 (m, 1H), 1.72-1.65 (m, 1H), 1.31 (s, 3H), 1.21 (s, 3H), 1.00 (s, 3H). 1H NMR (400 MHz, DMSO-d6) δ 10.07 (s, 1H), 9.20 (d, J = 2.0 Hz, 1H), 8.39 (d, J = 8.4 Hz, 1H), 8.19 (s, 1H), 8.10 (s, 1H), 7.85-7.82 (m, 1H), 7.12 (d, J = 7.2 Hz, 1H), 4.75-4.74 (m, 1H), 3.81-3.63(m, | LCMS (ES, m/z): 447 [M + H]+. Rt 0.561 min. LCMS (ES, m/z): 447 [M + H]+. Rt 0.515 min. |
| | | 5H), 2.53 (s, 3H), 2.35 (s, 3H), 2.21-2.18 | |
| | | (m, 1H), 1.72-1.67 (m, 1H), 1.31(s, 3H), | |
| | | 1.22 (s, 3H), 1.00 (s, 3H). | |
| 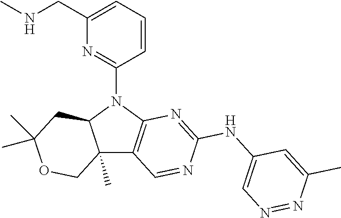 | | | |
| 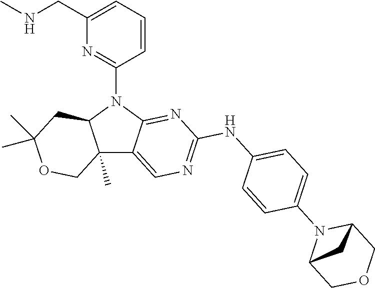 [SMILES: CNCc1cccc(N2c3nc(Nc4ccc(N5[C@@H]6COC[C@H]5C6)cc4)ncc3[C@@]3(C)COC(C)(C)C[C@@H]23)n1] | 479, 480 | 1H NMR (400 MHz, DMSO-d6) δ 9.00 (s, 1H), 8.48 (d, J = 8.4 Hz, 1H), 7.98 (d, J = 8.4 Hz, 1H), 7.71 (t, J = 7.9 Hz, 1H), 7.48 (d, J = 8.4 Hz, 2H), 7.03 (d, J = 7.6 Hz, 1H), 6.53 (d, J = 8.8 Hz, 2H), 4.69-4.66 (m, 1H), 4.22-4.19 (m, 4H), 3.88-3.72 (m, 3H), 3.66-3.55 (m, 3H), 2.67-2.62 (m, 1H), 2.35 (s, 3H), 2.22-2.17 (m, 1H), 1.83 (d, J = 8.0 Hz, 1H), 1.64-1.59 (m, 1H), 1.27 (s, 3H), 1.23 (s, 3H), 1.01 (s, 3H). 1H NMR (400 MHz, DMSO-d6) δ 9.00 (s, 1H), 8.48 (d, J = 8.4 Hz, 1H), 7.98 (d, J = 7.6 Hz, 1H), 7.71 (t, J = 8.0 Hz, 1H), 7.48 (d, J = 8.4 Hz, 2H), 7.03 (d, J = 7.2 Hz, 1H), 6.53 (d, J = 8.8 Hz, 2H), 4.69-4.66 (m, 1H), 4.22-4.19 (m, 4H), 3.77-3.72 (m, 3H), 3.66-3.58 (m, 3H), 2.66-2.62 | LCMS (ES, m/z): 528 [M + H]+, Rt 0.551 min. LCMS (ES, m/z): 528 [M + H]+, Rt 0.553 min. |
| | | (m, 1H), 2.35 (s, 3H), 2.21-2.17 (m, 1H), | |
| | | 1.83 (d, J = 8.0 Hz, 1H), 1.64-1.58 (m, | |
| | | 1H), 1.27 (s, 3H), 1.22 (s, 3H), 1.01 (s, | |
| | | 3H). | |
| 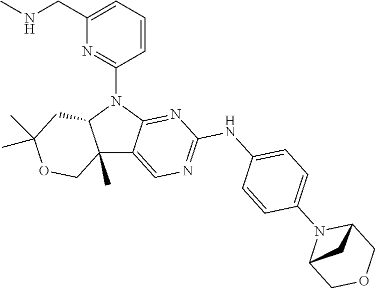 [SMILES: CNCc1cccc(N2c3nc(Nc4ccc(N5[C@@H]6COC[C@H]5C6)cc4)ncc3[C@]3(C)COC(C)(C)C[C@H]23)n1] | | | |
| 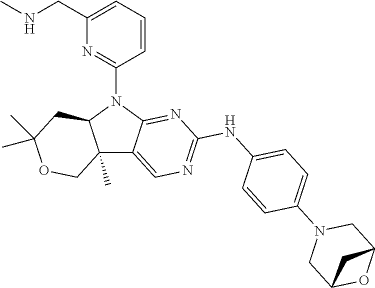 [SMILES: CNCc1cccc(N2c3nc(Nc4ccc(N5C[C@H]6C[C@@H](C5)O6)cc4)ncc3[C@@]3(C)COC(C)(C)C[C@@H]23)n1] | 481, 482 | 1H NMR (400 MHz, DMSO-d6) δ 8.98 (s, 1H), 8.51 (d, J = 8.4 Hz, 1H), 7.96 (s, 1H), 7.73 (t, J = 8 Hz, 1H), 7.54 (d, J = 7.6 Hz, 2H), 7.02 (d, J = 7.2 Hz, 1H), 6.71 (d, J = 8.8 Hz, 2H), 4.72-4.67 (m, 3H), 3.81-3.74 (m, 3H), 3.66-3.63 (m, 1H), 3.56-3.54 (m, 2H), 3.37-3.34 (m, 2H), 3.14-3.10 (m, 1H), 2.34 (s, 3H), 2.22- 2.17 (m, 1H), 1.96 (d, J = 8.0 Hz, 1H), 1.64-1.61 (m, 1H), 1.27 (s, 3H), 1.22 (s, 3H), 1.01 (s, 3H). 1H NMR (400 MHz, DMSO-d6) δ 8.99 (s, 1H), 8.52 (d, J = 8.0 Hz, 1H), 7.97(s, 1H), 7.74 (t, J = 8.0 Hz, 1H), 7.54 (d, J = 8.8 Hz, 2H), 7.03 (d, J = 7.2 Hz, 1H), 6.71 (d, J = 8.8 Hz, 2H), 4.72-4.67 (m, 3H), 3.81- 3.74 (m, 3H), 3.66-3.3.63 (m, 1H), 3.56- 3.54 (m, 2H), 3.37-3.34 (m, 2H), 3.14- | LCMS (ES, m/z): 528 [M + H]+. Rt1. 1.192 min. LCMS (ES, m/z): 528 [M + H]+. Rt1.0.575 min. |
| | | 3.10 (m, 1H), 2.38 (s, 3H), 2.22-2.17 | |
| | | (m, 1H), 1.96 (d, J = 8.0 Hz, 1H), 1.64- | |
| | | 1.61 (m, 1H), 1.27 (s, 3H), 1.22 (s, 3H), | |
| | | 1.01 (s, 3H). | |
| 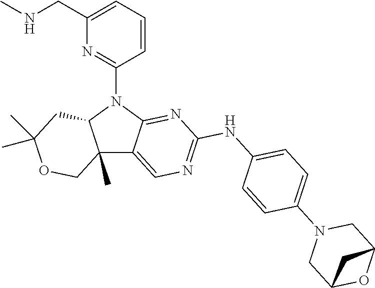 [SMILES: CNCc1cccc(N2c3nc(Nc4ccc(N5C[C@H]6C[C@@H](C5)O6)cc4)ncc3[C@]3(C)COC(C)(C)C[C@H]23)n1] | | | |
| 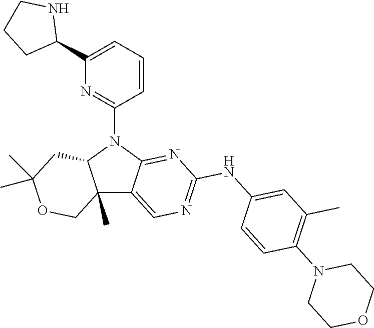 [SMILES: Cc1cc(Nc2ncc3c(n2)N(c2cccc([C@H]4CCCN4)n2)[C@H]2CC(C)(C)OC[C@@]32C)ccc1N1CCOCC1] | 521, 522 | 1H NMR (300 MHz, DMSO-d6) δ 9.18 (s, 1H), 8.49 (d, J = 8.1 Hz, 1H), 8.01 (s, 1H), 7.78-7.68 (m, 1H), 7.62 (d, J = 2.1 Hz, 1H), 7.43 (d, J = 9 Hz, 1H), 7.13 (d, J = 7.5 Hz, 1H), 7.00 (d, J = 9.3 Hz, 1H), 4.69-4.64 (m, 1H), 4.13 (t, J = 6.6 Hz, 1H), 3.84-3.63 (m, 6H), 3.02-2.75 (m, 7H), 2.28-2.12 (m, 5H), 1.75-1.60 (m, 4H), 1.30 (s, 3H), 1.25 (s, 3H), 1.05 (s, 3H). 1H NMR (300 MHz, DMSO-d6) δ 9.19 (s, 1H), 8.49 (d, J = 8.4 Hz, 1H), 8.02 (s, 1H), 7.77-7.69 (m, 1H), 7.62 (d, J = 2.4 Hz, 1H), 7.45-7.42 (m, 1H), 7.08 (d, J = 7.2 Hz, 1H), 7.00 (d, J = 8.6 Hz, 1H), 4.71-4.66 (m, 1H), 4.11 (d, J = 6.9 Hz, 1H), 3.81-3.65 (m, 6H), 3.11-3.02 (m, 1H), 2.89-2.80 (m, 6H), 2.28-2.21(m, 5H), 1.78-1.62 (m, 4H), 1.27 (d, J = 17.7 Hz, 6H), 1.05 (s, 3H). | LCMS (ES, m/z): 556.35 [M + H]+, Rt 0.787 min. LCMS (ES, m/z): 556.35 [M + H]+, Rt 0.803 min. |
| 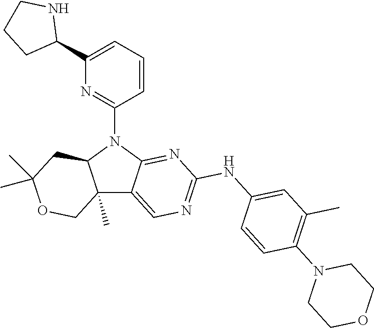 [SMILES: Cc1cc(Nc2ncc3c(n2)N(c2cccc([C@H]4CCCN4)n2)[C@@H]2CC(C)(C)OC[C@]32C)ccc1N1CCOCC1] | | | |
| 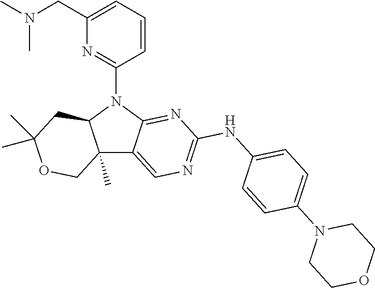 [SMILES: CN(C)Cc1cccc(N2c3nc(Nc4ccc(N5CCOCC5)cc4)ncc3[C@@]3(C)COC(C)(C)C[C@@H]23)n1] | 523, 524 | 1H NMR (300 MHz, DMSO-d6) δ 9.11 (s, 1H), 8.51 (d, J = 8.4 Hz, 1H), 8.00 (s, 1H), 7.83-7.72 (m, 1H), 7.58 (d, J = 9.0 Hz, 2H), 7.07 (d, J = 7.5 Hz, 1H), 6.96- 6.87 (m, 2H), 4.70-4.60 (m, 1H), 3.82- 3.72 (m, 5H), 3.70-3.60 (m, 1H), 3.60- 3.43 (m, 2H), 3.10-3.02 (m, 4H), 2.30- 2.15 (m, 7H), 1.72-1.59 (m, 1H), 1.28 (s, 3H), 1.22 (s, 3H), 1.01 (s, 3H). 1H NMR (300 MHz, DMSO-d6) δ 9.11 (s, 1H), 8.51 (d, J = 8.4 Hz, 1H), 8.00 (s, 1H), 7.83-7.72 (m, 1H), 7.58 (d, J = 9.0 Hz, 2H), 7.07 (d, J = 7.2 Hz, 1H), 6.92 (d, J = 9.0 Hz, 2H), 4.70-4.60 (m, 1H), 3.82- 3.72 (m, 5H), 3.70-3.60 (m, 1H), 3.57- 3.43 (m, 2H), 3.10-3.02 (m, 4H), 2.27- 2.15 (m, 7H), 1.72-1.59 (m, 1H), 1.28 (s, 3H), 1.22 (s, 3H), 1.01 (s, 3H). | LCMS (ES, m/z): 530 [M + H]+, Rt 0.575 min. LCMS (ES, m/z): 530 [M + H]+, Rt 0.567 min. |
| 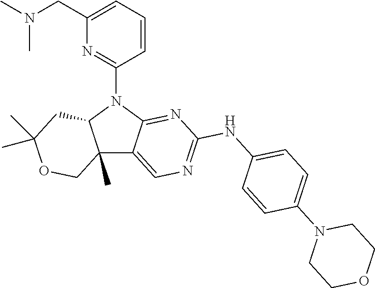 [SMILES: CN(C)Cc1cccc(N2c3nc(Nc4ccc(N5CCOCC5)cc4)ncc3[C@]3(C)COC(C)(C)C[C@H]23)n1] | | | |
| 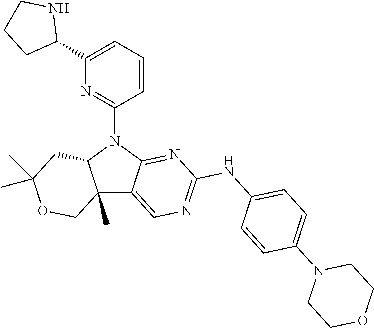 [SMILES: CC1(C)C[C@@H]2N(c3cccc([C@@H]4CCCN4)n3)c3nc(Nc4ccc(N5CCOCC5)cc4)ncc3[C@]2(C)CO1] | 525, 526 | 1H NMR (300 MHz, DMSO-d6) δ 9.11 (s, 1H), 8.50 (d, J = 8.4 Hz, 1H), 8.00 (d, J = 6.3 Hz, 1H), 7.79-7.68 (m, 1H), 7.59 (d, J = 9.0 Hz, 2H), 7.12 (d, J = 7.2 Hz, 1H), 6.92 (d, J = 9.0 Hz, 2H), 4.70-4.60 (m, 1H), 4.17-4.07 (m, 1H), 3.82-3.72 (m, 5H), 3.70-3.62 (m, 1H), 3.10-2.96 (m, 5H), 2.96-2.84 (m, 1H), 2.29-2.06 (m, 2H), 1.79-1.68 (m, 3H), 1.65-1.50 (m, 1H), 1.29 (s, 3H), 1.25 (s, 3H), 1.05 (s, 3H). 1H NMR (300 MHz, DMSO-d6) δ 9.11 (s, 1H), 8.50 (d, J = 8.4 Hz, 1H), 7.99 (s, 1H), 7.78-7.68 (m, 1H), 7.58 (d, J = 9.0 Hz, 2H), 7.07 (d, J = 7.5 Hz, 1H), 6.92 (d, J = 9.0 Hz, 2H), 4.72-4.62 (m, 1H), 4.14- 4.05 (m, 1H), 3.82-3.72 (m, 5H), 3.70- 3.62 (m, 1H), 3.10-3.00 (m, 5H), 2.92- 2.81 (m, 1H), 2.30-2.18 (m, 2H), 2.15- 2.05 (m, 1H), 1.80-1.70 (m, 3H), 1.67- | LCMS (ES, m/z): 542.30 [M + H]+, Rt 0.583 min. LCMS (ES, m/z): 542.30 [M + H]+, Rt 0.600 min. |
| | | 1.55 (m, 1H), 1.30 (s, 3H), 1.24 (s, 3H), | |
| | | 1.04 (s, 3H). | |
| 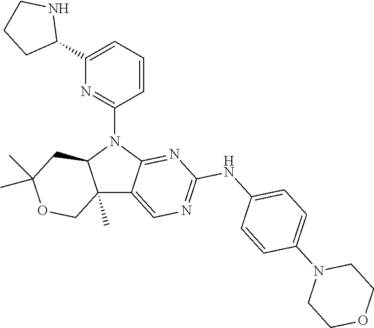 [SMILES: CC1(C)C[C@H]2N(c3cccc([C@@H]4CCCN4)n3)c3nc(Nc4ccc(N5CCOCC5)cc4)ncc3[C@@]2(C)CO1] | | | |
| 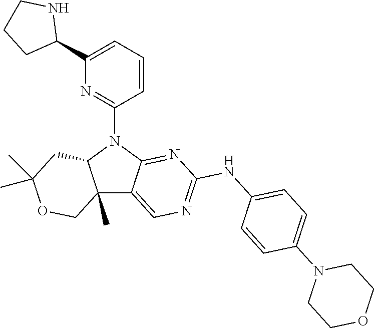 [SMILES: CC1(C)C[C@@H]2N(c3cccc([C@H]4CCCN4)n3)c3nc(Nc4ccc(N5CCOCC5)cc4)ncc3[C@]2(C)CO1] | 527, 528 | 1H NMR (400 MHz, DMSO-d6) δ 9.12 (s, 1H), 8.49 (d, J = 8.4 Hz, 1H), 7.98 (s, 1H), 7.72 (t, J = 7.6 Hz, 1H), 7.57 (d, J = 8.7 Hz, 2H), 7.06 (d, J = 7.6 Hz, 1H), 6.94-6.87 (m, 2H), 4.71-4.63 (m, 1H), 4.13-4.05 (m, 1H), 3.81-3.71 (m, 5H), 3.70-3.62 (m, 1H), 3.10-3.00 (m, 5H), 2.91-2.80 (m, 1H), 2.57-2.53 (m, 1H), 2.27-2.18 (m, 1H), 2.15-2.05 (m, 1H), 1.81-1.67 (m, 3H), 1.64-1.54 (m, 1H), 1.29 (s, 3H), 1.23 (s, 3H), 1.03 (s, 3H). 1H NMR (300 MHz, DMSO-d6) δ 9.14 (s, 1H), 8.60 (d, J = 8.4 Hz, 1H), 8.20 (s, 1H), 8.00 (s, 1H), 7.81 (t, J = 7.8 Hz, 1H), 7.59-7.50 (m, 2H), 7.11 (d, J = 7.5 Hz, 1H), 6.94-6.84 (m, 2H), 4.77-4.66 (m, 1H), 4.64-4.55 (m, 1H), 3.81-3.77 (m, 1H), 3.77-3.75 (m, 1H), 3.75-3.69 (m, 4H), 3.69-3.65 (m, 1H), 3.64-3.61 (m, 1H), 3.07-2.98 (m, 4H), 2.42-2.20 (m, | LCMS (ES, m/z): 542.25 [M + H]+, Rt 0.617 min. LCMS (ES, m/z): 542.25 [M + H]+, Rt 0.833 min. |
| | | 3H), 2.02-1.89 (m, 3H), 1.61-1.48 (m, | |
| | | 1H), 1.26 (s, 3H), 1.23 (s, 3H), 1.01 (s, | |
| | | 3H) | |
| 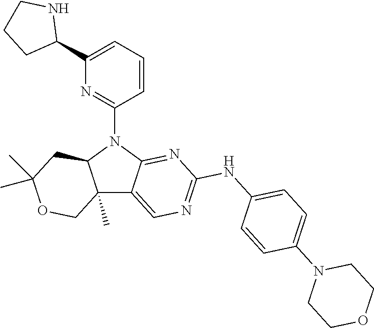 [SMILES: CC1(C)C[C@H]2N(c3cccc([C@H]4CCCN4)n3)c3nc(Nc4ccc(N5CCOCC5)cc4)ncc3[C@@]2(C)CO1] | | | |
| 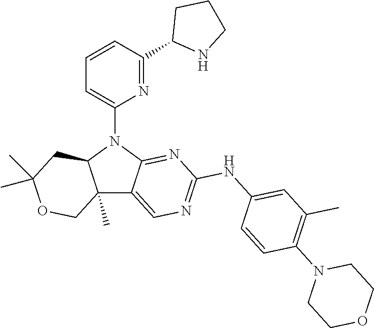 [SMILES: Cc1cc(Nc2ncc3c(n2)N(c2cccc([C@@H]4CCCN4)n2)[C@@H]2CC(C)(C)OC[C@]32C)ccc1N1CCOCC1] | 529, 530 | 1H NMR (300 MHz, DMSO-d6) δ 9.17 (s, 1H), 8.46 (d, J = 8.4 Hz, 1H), 7.98 (s, 1H), 7.69 (t, J = 7.8 Hz, 1H), 7.59 (d, J = 2.4 Hz, 1H), 7.40 (d, J = 9.0 Hz, 1H), 7.09 (d, J = 7.5 Hz, 1H), 6.96 (d, J = 8.7 Hz, 1H), 4.68-4.58 (m, 1H), 4.10 (t, J = 6.6 Hz, 1H), 3.81-3.61 (m, 6H), 3.05-2.93 (m, 1H), 2.93-2.82 (m, 1H), 2.86-2.73 (m, 4H), 2.24 (s, 4H), 2.23-2.14 (m, 1H), 2.14-2.03 (m, 1H), 1.77-1.65 (m, 3H), 1.63-1.50 (m, 1H), 1.27 (s, 3H), 1.21 (s, 3H), 1.01 (s, 3H). 1H NMR (300 MHz, DMSO-d6) δ 9.17 (s, 1H), 8.46 (d, J = 8.4 Hz, 1H), 7.98 (s, 1H), 7.69 (t, J = 7.8 Hz, 1H), 7.58 (d, J = 2.7 Hz, 1H), 7.40 (d, J = 8.7 Hz, 1H), 7.04 (d, J = 7.5 Hz, 1H), 6.96 (d, J = 8.7 Hz, 1H), 4.70-4.60 (m, 1H), 4.06 (t, J = 6.6 Hz, 1H), 3.81-3.61 (m, 6H), 3.05-2.99 (m, 1H), 2.90-2.74 (m, 5H), 2.53-2.50 | LCMS (ES, m/z): 556.25 [M + H]+, Rt 0.633 min. LCMS (ES, m/z): 556.25 [M + H]+, Rt 0.658 min. |
| | | (m, 1H), 2.24 (s, 3H), 2.23-2.16 (m, 1H), | |
| | | 2.14-2.07 (m, 1H), 1.80-1.68 (m, 3H), | |
| | | 1.64-1.50 (m, 1H), 1.27 (s, 3H), 1.21 (s, | |
| | | 3H), 1.01 (s, 3H). | |
| 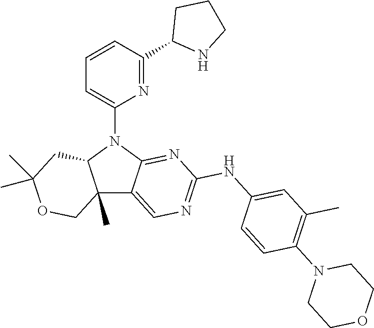 [SMILES: Cc1cc(Nc2ncc3c(n2)N(c2cccc([C@@H]4CCCN4)n2)[C@H]2CC(C)(C)OC[C@@]32C)ccc1N1CCOCC1] | | | |
| 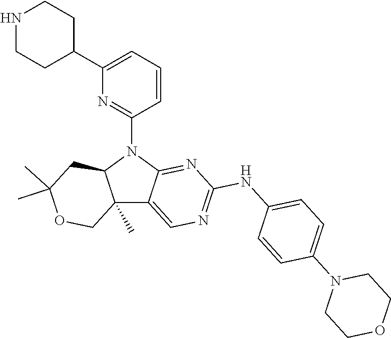 [SMILES: CC1(C)C[C@H]2N(c3cccc(C4CCNCC4)n3)c3nc(Nc4ccc(N5CCOCC5)cc4)ncc3[C@@]2(C)CO1] | 531 532 | 1H NMR (300 MHz, DMSO-d6) δ 9.09 (s, 1H), 8.46 (d, J = 8.4 Hz, 1H), 7.98 (s, 1H), 7.76-7.65 (m, 1H), 7.58 (d, J = 9.0 Hz, 2H), 6.96-6.89 (m, 3H), 4.70-4.60 (m, 1H), 3.82-3.62 (m, 6H), 3.10-3.01 (m, 6H), 2.77-2.54 (m, 3H), 2.30-2.20 (m, 1H), 1.86-1.75 (m, 2H), 1.71-1.55 (m, 3H), 1.30 (s, 3H), 1.25 (s, 3H), 1.04 (s, 3H). 1H NMR (300 MHz, DMSO-d6) δ 9.09 (s, 1H), 8.46 (d, J = 8.4 Hz, 1H), 7.98 (s, 1H), 7.76-7.65 (m, 1H), 7.58 (d, J = 9.0 Hz, 2H), 6.97-6.88 (m, 3H), 4.72-4.62 (m, 1H), 3.80-3.60 (m, 6H), 3.10-3.00 (m, 6H), 2.77-2.53 (m, 3H), 2.30-2.20 (m, 1H), 1.85-1.75 (m, 2H), 1.71- 1.55 (m, 3H), 1.30 (s, 3H), 1.25 (s, 3H), 1.04 (s, 3H). | LCMS (ES, m/z): 556.30 [M + H]+, Rt 0.750 min. LCMS (ES, m/z): 556.30 [M + H]+, Rt 0.742 min. |
| 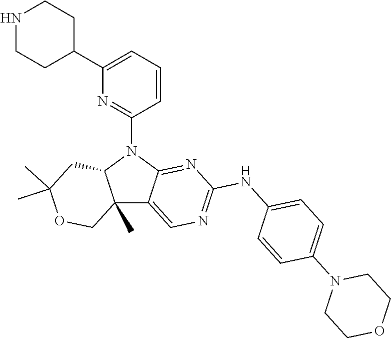 [SMILES: CC1(C)C[C@@H]2N(c3cccc(C4CCNCC4)n3)c3nc(Nc4ccc(N5CCOCC5)cc4)ncc3[C@]2(C)CO1] | | | |
| 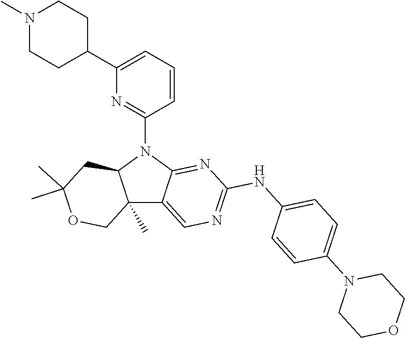 [SMILES: CN1CCC(c2cccc(N3c4nc(Nc5ccc(N6CCOCC6)cc5)ncc4[C@@]4(C)COC(C)(C)C[C@@H]34)n2)CC1] | 533, 534 | 1H NMR (300 MHz, DMSO-d6) δ 9.09 (s, 1H), 8.47 (d, J = 8.4 Hz, 1H), 7.98 (s, 1H), 7.76-7.65 (m, 1H), 7.58 (d, J = 8.7 Hz, 2H), 6.97-6.87 (m, 3H), 4.71-4.61 (m, 1H), 3.80-3.62 (m, 6H), 3.10-3.01 (m, 4H), 2.92-2.82 (m, 2H), 2.65-2.53 (m, 1H), 2.30-2.16 (m, 4H), 2.04-1.92 (m, 2H), 1.88-1.72 (m, 4H), 1.69-1.58 (m, 1H), 1.30 (s, 3H), 1.24 (s, 3H), 1.03 (s, 3H). 1H NMR (300 MHz, DMSO-d6) δ 9.09 (s, 1H), 8.47 (d, J = 8.7 Hz, 1H), 7.98 (s, 1H), 7.76-7.67 (m, 1H), 7.58 (d, J = 9.0 Hz, 2H), 6.97-6.87 (m, 3H), 4.71-4.61 (m, 1H), 3.81-3.61 (m, 6H), 3.09-3.01 (m, 4H), 2.92-2.83 (m, 2H), 2.65-2.53 (m, 1H), 2.30-2.17 (m, 4H), 2.04-1.92 (m, 2H), 1.89-1.72 (m, 4H), 1.70-1.58 (m, 1H), 1.30 (s, 3H), 1.24 (s, 3H), 1.03 (s, 3H). | LCMS (ES, m/z): 570.35 [M + H]+, Rt 2.128 min. LCMS (ES, m/z): 570.35 [M + H]+, Rt 0.783 min. |
| 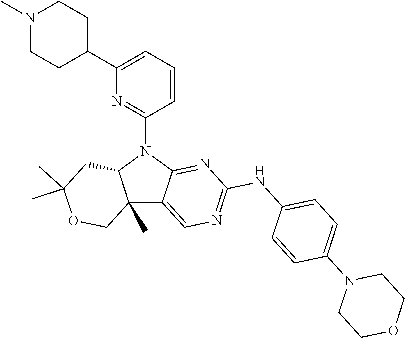 [SMILES: CN1CCC(c2cccc(N3c4nc(Nc5ccc(N6CCOCC6)cc5)ncc4[C@]4(C)COC(C)(C)C[C@H]34)n2)CC1] | | | |
| 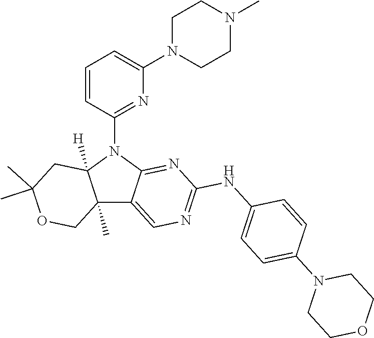 [SMILES: CN1CCN(c2cccc(N3c4nc(Nc5ccc(N6CCOCC6)cc5)ncc4[C@@]4(C)COC(C)(C)C[C@@H]34)n2)CC1] | 535, 536 | 1H NMR (300 MHz, DMSO-d6) δ 9.05 (s, 1H), 8.08-7.94 (m, 2H), 7.60-7.52 (m, 3H), 6.91 (d, J = 8.7 Hz, 2H), 6.46 (d, J = 8.1 Hz, 1H), 4.61-4.56 (m, 1H), 3.77- 3.76 (m, 5H), 3.68-3.64 (m, 1H), 3.60- 3.50 (m, 4H), 3.07-3.04 (m, 4H), 2.52- 2.42 (m, 4H), 2.30-2.01(m, 4H), 1.58- 1.44 (m, 1H), 1.29 (s, 3H), 1.23 (s, 3H), 1.07 (s, 3H). 1H NMR (300 MHz, DMSO-d6) δ 9.06 (s, 1H), 7.98-7.94 (m, 2H), 7.61-7.52 (m, 3H), 6.91 (d, J = 8.4 Hz, 2H), 6.46 (d, J = 8.1 Hz, 1H), 4.61-4.58 (m, 1H), 4.00- 3.76 (m, 5H), 3.68-3.64 (m, 1H), 3.61- 3.55 (m, 4H), 3.06-2.95 (m, 4H), 2.52- 2.42 (m, 4H), 2.29-2.13 (m, 4H), 1.58- 1.50 (m, 1H), 1.29 (s, 3H), 1.24 (s, 3H), 1.08 (s, 3H). | LCMS (ES, m/z): 571.35 [M + H]+, Rt 0.758 min. LCMS (ES, m/z): 571.35 [M + H]+, Rt 0.758 min. |
| 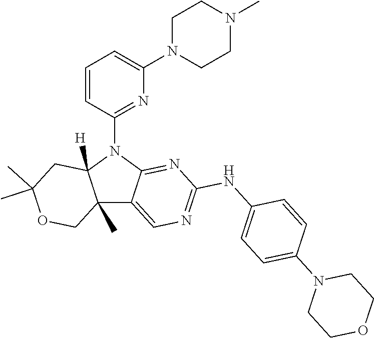 [SMILES: CN1CCN(c2cccc(N3c4nc(Nc5ccc(N6CCOCC6)cc5)ncc4[C@]4(C)COC(C)(C)C[C@H]34)n2)CC1] | | | |
| 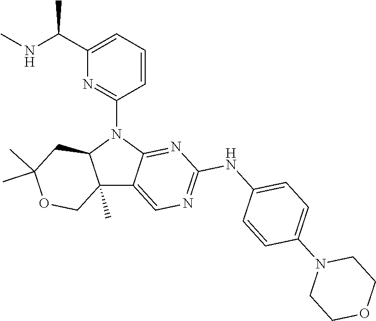 [SMILES: CN[C@@H](C)c1cccc(N2c3nc(Nc4ccc(N5CCOCC5)cc4)ncc3[C@@]3(C)COC(C)(C)C[C@@H]23)n1] | 537, 538 | 1H NMR (300 MHz, DMSO-d6) δ 9.10 (s, 1H), 8.49 (d, J = 8.1 Hz, 1H), 7.99 (s, 1H), 7.75 (t, J = 7.8 Hz, 1H), 7.58 (d, J = 8.7 Hz, 2H), 7.03 (d, J = 7.5 Hz, 1H), 6.92 (d, J = 9.0 Hz, 2H), 4.72-4.67 (m, 1H), 3.76 (s, 5H), 3.68-3.55 (m, 2H), 3.11- 3.01 (m, 4H), 2.29-2.16 (m, 4H), 1.69- 1.61 (m, 1H), 1.32-1.24 (m, 9H), 1.03 (s, 3H). 1H NMR (300 MHz, DMSO-d6) δ 9.11 (s, 1H), 8.49 (d, J = 8.4 Hz, 1H), 7.99 (s, 1H), 7.75 (d, J = 8.4 Hz, 1H), 7.58 (d, J = 9.0 Hz, 2H), 7.06 (d, J = 7.2 Hz, 1H), 6.91 (d, J = 9.0 Hz, 2H), 4.71-4.66 (m, 1H), 3.78-3.74 (m, 5H), 3.68-3.57 (m, 2H), 3.07-3.04 (m, 4H), 2.28-2.16 (m, 4H), 1.69-1.64 (m, 1H), 1.35-1.18 (m, 9H), 1.02 (s, 3H). | LCMS (ES, m/z): 530.25 [M + H]+, Rt 0.617 min. LCMS (ES, m/z): 530.25 [M + H]+, Rt 0.592 min. |
| 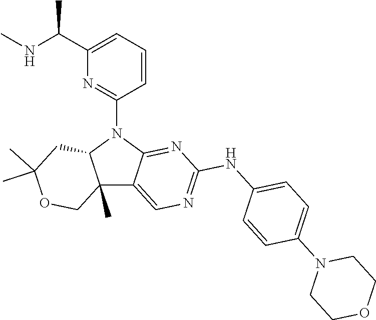 [SMILES: CN[C@@H](C)c1cccc(N2c3nc(Nc4ccc(N5CCOCC5)cc4)ncc3[C@]3(C)COC(C)(C)C[C@H]23)n1] | | | |
| 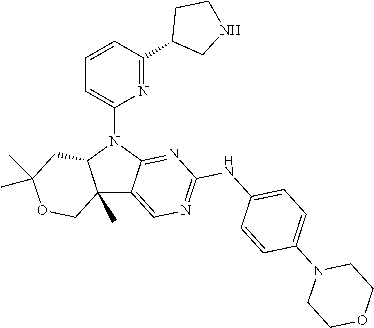 [SMILES: CC1(C)C[C@@H]2N(c3cccc([C@@H]4CCNC4)n3)c3nc(Nc4ccc(N5CCOCC5)cc4)ncc3[C@]2(C)CO1] | 539, 540 | 1H NMR (300 MHz, DMSO-d6) δ 9.09 (s, 1H), 8.49 (d, J = 8.4 Hz, 1H), 7.98 (s, 1H), 7.68 (t, J = 7.8 Hz, 1H), 7.59 (d, J = 9.0 Hz, 2H), 6.94-6.90 (m, 3H), 4.66- 4.62 (m, 1H), 3.81-3.65 (m, 6H), 3.53- 3.40 (m, 1H), 3.27-3.14 (m, 2H), 3.07- 3.04 (m, 4H), 2.99-2.76 (m, 2H), 2.29- 2.21 (m, 1H), 2.13-1.91 (m, 2H), 1.62- 1.55 (m, 1H), 1.29 (s, 3H), 1.24 (s, 3H), 1.05 (s, 3H). 1H NMR (300 MHz, DMSO-d6) δ 9.09 (s, 1H), 8.48 (d, J = 8.7 Hz, 1H), 7.98 (s, 1H), 7.68 (t, J = 7.8 Hz, 1H), 7.59 (d, J = 9.0 Hz, 2H), 6.94-6.90 (m, 3H), 4.66- 4.62 (m, 1H), 3.81-3.65 (m, 6H), 3.27- 3.14 (m, 2H), 3.07-3.04 (m, 4H), 2.99- 2.76 (m, 3H), 2.29-2.21 (m, 1H), 2.13- 1.91 (m, 2H), 1.62-1.55 (m, 1H), 1.29 (s, 3H), 1.24 (s, 3H), 1.05 (s, 3H). | LCMS (ES, m/z): 542.30 [M + H]+, Rt 0.733 min. LCMS (ES, m/z): 542.25 [M + H]+, Rt 0.625 min. |
| 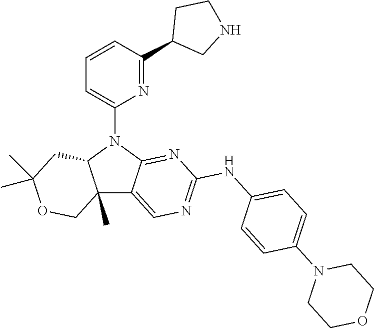 [SMILES: CC1(C)C[C@@H]2N(c3cccc([C@H]4CCNC4)n3)c3nc(Nc4ccc(N5CCOCC5)cc4)ncc3[C@]2(C)CO1] | | | |
| 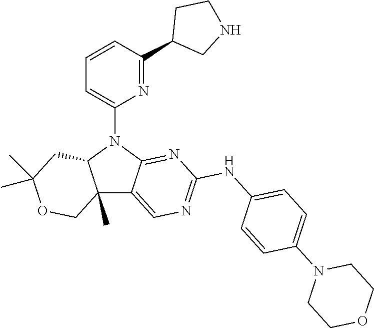 [SMILES: CC1(C)C[C@@H]2N(c3cccc([C@H]4CCNC4)n3)c3nc(Nc4ccc(N5CCOCC5)cc4)ncc3[C@]2(C)CO1] | 541, 542 | 1H NMR (300 MHz, DMSO-d6) δ 9.09 (s, 1H), 8.48 (d, J = 8.1 Hz, 1H), 7.98 (s, 1H), 7.68 (t, J = 7.5 Hz, 1H), 7.59 (d, J = 8.7 Hz, 2H), 6.94-6.90 (m, 3H), 4.66-4.61 (m, 1H), 3.80-3.74 (m, 5H), 3.69-3.65 (m, 1H), 3.52-3.47 (m, 1H), 3.27-3.16 (m, 2H), 3.07-3.04 (m, 4H), 2.97-2.91 (m, 2H), 2.29-2.21 (m, 1H), 2.13-1.93 (m, 2H), 1.63-1.55 (m, 1H), 1.29 (s, 3H), 1.25 (s, 3H), 1.05 (s, 3H). 1H NMR (300 MHz, DMSO-d6) δ 9.09 (s, 1H), 8.49 (d, J = 8.4 Hz, 1H), 7.98 (s, 1H), 7.68 (t, J = 7.8 Hz, 1H), 7.59 (d, J = 9.0 Hz, 2H), 6.94-6.90 (m, 3H), 4.66-4.62 (m, 1H), 3.81-3.65 (m, 6H), 3.53-3.40 (m, 1H), 3.27-3.14 (m, 2H), 3.07-3.04 (m, 4H), 2.98-2.91 (m, 1H), 2.85-2.82 (m, 1H), 2.29-2.21 (m, 1H), 2.15-2.03 (m, 1H), 2.00-1.88 (m, 1H), 1.62-1.55 (m, 1H), 1.29 (s, 3H), 1.24 (s, 3H), 1.05 | LCMS (ES, m/z): 542.30 [M + H]+, Rt 0.625 min. LCMS (ES, m/z): 542.30 [M + H]+, Rt 0.642 min. |
| | | (s, 3H). | |
| 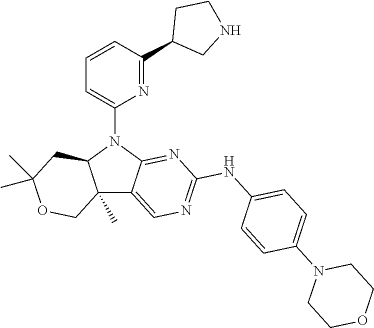 [SMILES: CC1(C)C[C@H]2N(c3cccc([C@H]4CCNC4)n3)c3nc(Nc4ccc(N5CCOCC5)cc4)ncc3[C@@]2(C)CO1] | | | |
| 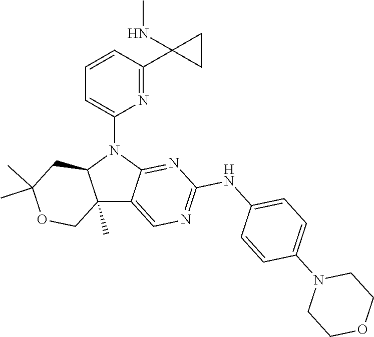 [SMILES: CNC1(c2cccc(N3c4nc(Nc5ccc(N6CCOCC6)cc5)ncc4[C@@]4(C)COC(C)(C)C[C@@H]34)n2)CC1] | 543, 544 | 1H NMR (400 MHz, DMSO-d6) δ 9.08 (s, 1H), 8.41 (d, J = 8.4 Hz, 1H), 7.95 (s, 1H), 7.71 (t, J = 8.0 Hz, 1H), 7.57 (d, J = 8.8 Hz, 2H), 7.28 (d, J = 7.6 Hz, 1H), 6.90 (d, J = 8.8 Hz, 2H), 4.54-4.50 (m, 1H), 3.77-3.73 (m, 5H), 3.66-3.63 (m 1H), 3.05-3.03 (m, 4H), 2.30 (s, 3H), 2.17- 2.12 (m, 1H), 1.53-1.47 (m, 1H), 1.34- 1.26 (m, 8H), 1.15-1.09 (m, 1H), 1.06- 0.91 (m, 5H). 1H NMR (400 MHz, DMSO-d6) δ 9.08 (s, 1H), 8.41 (d, J = 8.4 Hz, 1H), 7.95 (s, 1H), 7.71 (t, J = 8.0 Hz, 1H), 7.56 (d, J = 9.2 Hz, 2H), 7.28 (d, J = 7.6 Hz, 1H), 6.90 (d, J = 8.8 Hz, 2H), 4.54-4.50 (m, 1H), 3.77-3.73 (m, 5H), 3.66-3.63 (m 1H), 3.05-3.03 (m, 4H), 2.30 (s, 3H), 2.17- 2.12 (m, 1H), 1.53-1.47 (m, 1H), 1.32- 1.28 (m, 1H), 1.26-1.23 (m, 7H), 1.15- 1.11 (m, 1H), 1.06-0.91 (m, 5H). | LCMS (ES, m/z): 542.30 [M + H]+, Rt 0.617 min. LCMS (ES, m/z): 542.30 [M + H]+, Rt 0.617 min. |
| 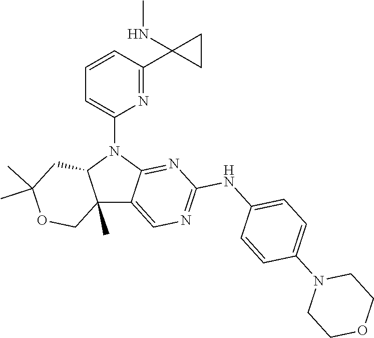 [SMILES: CNC1(c2cccc(N3c4nc(Nc5ccc(N6CCOCC6)cc5)ncc4[C@]4(C)COC(C)(C)C[C@H]34)n2)CC1] | | | |
| 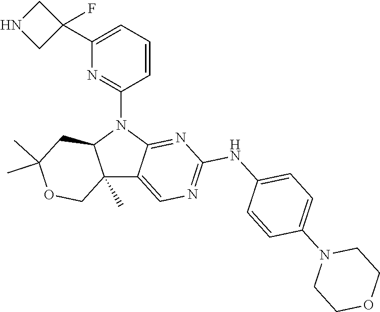 [SMILES: CC1(C)C[C@H]2N(c3cccc(C4(F)CNC4)n3)c3nc(Nc4ccc(N5CCOCC5)cc4)ncc3[C@@]2(C)CO1] | 545, 546 | 1H NMR (300 MHz, DMSO-d6) δ 9.16 (s, 1H), 8.70 (d, J = 8.4 Hz, 1H), 8.03 (s, 1H), 7.87 (t, J = 7.5 Hz, 1H), 7.60 (d, J = 9.0 Hz, 2H), 7.17 (d, J = 7.5 Hz, 1H), 6.94 (d, J = 9.0 Hz, 2H), 4.75-4.70 (m, 1H), 4.14-4.03 (m, 2H), 3.79-3.77 (m, 1H), 3.79-3.74 (m, 6H), 3.70-3.66 (m, 1H), 3.08-3.04 (m, 4H), 2.17-2.11 (m, 1H), 1.68-1.60 (m, 1H), 1.32 (s, 3H), 1.27 (s, 3H), 1.06 (s, 3H). 1H NMR (300 MHz, DMSO-d6) δ 9.16 (s, 1H), 8.70 (d, J = 8.4 Hz, 1H), 8.03 (s, 1H), 7.88 (t, J = 7.5 Hz, 1H), 7.60 (d, J = 7.5 Hz, 2H), 7.17 (d, J = 7.5 Hz, 1H), 6.94 (d, J = 9.0 Hz, 2H), 4.75-4.70 (m, 1H), 4.14-4.03 (m, 2H), 3.40-3.66 (m, 8H), 3.08-3.04 (m, 4H), 2.34-2.29 (m, 1H), 1.68-1.60 (m, 1H), 1.32 (s, 3H), 1.27 (s, 3H), 1.06 (s, 3H). | LCMS (ES, m/z): 546.25 [M + H]+, Rt 0.617 min. LCMS (ES, m/z): 546.25 [M + H]+, Rt 0.600 min. |
| 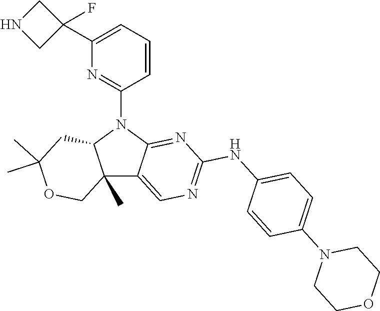 [SMILES: CC1(C)C[C@@H]2N(c3cccc(C4(F)CNC4)n3)c3nc(Nc4ccc(N5CCOCC5)cc4)ncc3[C@]2(C)CO1] | | | |
| 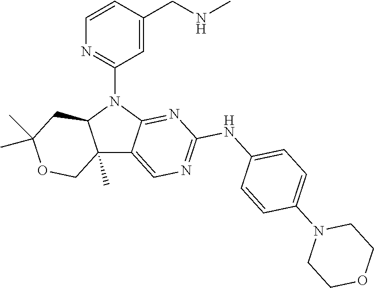 [SMILES: CNCc1ccnc(N2c3nc(Nc4ccc(N5CCOCC5)cc4)ncc3[C@@]3(C)COC(C)(C)C[C@@H]23)c1] | 547, 548 | 1H NMR (400 MHz, DMSO-d6) δ 9.05 (s, 1H), 8.44 (s, 1H), 8.27 (d, J = 4.8 Hz, 1H), 8.00 (s, 1H), 7.51 (d, J = 8.8 Hz, 2H), 7.04 (d, J = 4.8, 1H), 6.91 (d, J = 9.2 Hz, 2H), 4.69-4.66 (m, 1H), 3.81-3.78 (m, 1H), 3.75-3.73 (m, 4H), 3.67-3.62 (m, 4H), 3.06-3.03 (m, 4H), 2.27 (s, 3H), 2.18-2.13 (m, 1H), 1.60-1.54 (m, 1H), 1.23 (s, 3H), 1.20 (s, 3H), 1.00 (s, 3H). 1H NMR (400 MHz, DMSO-d6) δ 9.06 (s, 1H), 8.44 (s, 1H), 8.27 (d, J = 4.8 Hz, 1H), 8.00 (s, 1H), 7.51 (d, J = 8.8 Hz, 2H), 7.04 (d, J = 4.8, 1H), 6.91 (d, J = 9.2 Hz, 2H), 4.69-4.66 (m, 1H), 3.81-3.78 (m, 1H), 3.75-3.73 (m, 4H), 3.67-3.62 (m, 4H), 3.06-3.03 (m, 4H), 2.27 (s, 3H), 2.18-2.13 (m, 1H), 1.60-1.54 (m, 1H), 1.23 (s, 3H), 1.20 (s, 3H), 1.00 (s, 3H). | LCMS (ES, m/z): 516.30 [M + H]+, Rt 0.583 min. LCMS (ES, m/z): 516.30 [M + H]+, Rt 0.575 min. |
| 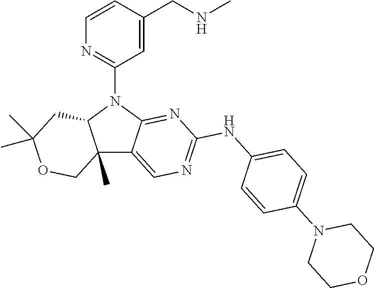 [SMILES: CNCc1ccnc(N2c3nc(Nc4ccc(N5CCOCC5)cc4)ncc3[C@]3(C)COC(C)(C)C[C@H]23)c1] | | | |
| 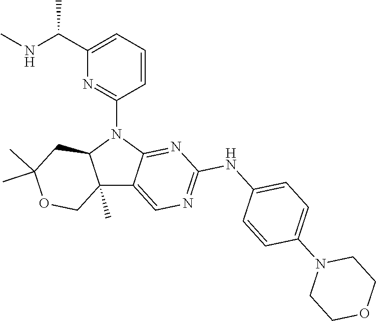 [SMILES: CN[C@H](C)c1cccc(N2c3nc(Nc4ccc(N5CCOCC5)cc4)ncc3[C@@]3(C)COC(C)(C)C[C@@H]23)n1] | 549, 550 | 1H NMR (300 MHz, DMSO-d6) δ 9.10 (s, 1H), 8.49 (d, J = 8.7 Hz, 1H), 7.99 (s, 1H), 7.75 (t, J = 8.1 Hz, 1H), 7.58 (d, J = 8.7 Hz, 2H), 7.06 (d, J = 7.2 Hz, 1H), 6.91 (d, J = 9.3 Hz, 2H), 4.71-4.66 (m, 1H), 3.77-3.74 (m, 5H), 3.68 (s, 1H), 3.64- 3.57 (m, 1H), 3.11-3.01 (m, 4H), 2.22 (s, 3H), 2.20-2.12 (s, 1H), 1.69-1.61 (m, 1H), 1.30-1.27 (m, 6H), 1.23 (s, 3H), 1.02 (s, 3H). 1H NMR (300 MHz, DMSO-d6) δ 9.10 (s, 1H), 8.49 (d, J = 8.4 Hz, 1H), 7.99 (s, 1H), 7.77-7.72 (m, 1H), 7.58 (d, J = 9.0 Hz, 2H), 7.03 (d, J = 7.5 Hz, 1H), 6.93- 6.90 (m, 2H), 4.71-4.67 (m, 1H), 3.78- 3.74 (m, 5H), 3.68-3.54 (m, 2H), 3.09- 3.01 (m, 4H), 2.29-2.17 (m, 4H), 2.07 (s, 1H), 1.69-1.61 (m, 1H), 1.32-1.29 (m, 6H), 1.24 (s, 3H), 1.03 (s, 3H). | LCMS (ES, m/z): 530.30 [M + H]+, Rt 0.600 min. LCMS (ES, m/z): 530.30 [M + H]+, Rt 0.617 min. |
| 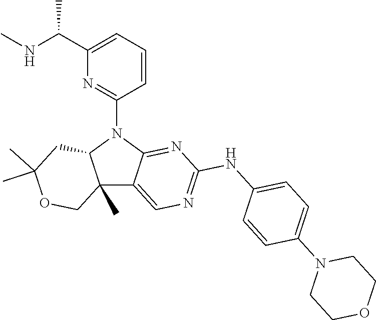 [SMILES: CN[C@H](C)c1cccc(N2c3nc(Nc4ccc(N5CCOCC5)cc4)ncc3[C@]3(C)COC(C)(C)C[C@H]23)n1] | | | |
| 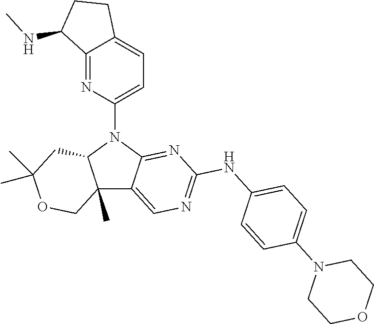 [SMILES: CN[C@H]1CCc2ccc(N3c4nc(Nc5ccc(N6CCOCC6)cc5)ncc4[C@]4(C)COC(C)(C)C[C@H]34)nc21] | 551 552 | 1H NMR (300 MHz, DMSO-d6) δ 9.08 (s, 1H), 8.45 (d, J = 8.4 Hz, 1H), 7.98 (s, 1H), 7.67-7.57 (m, 3H), 6.93 (d, J = 9.0 Hz, 2H), 4.69-4.65 (m, 1H), 3.93 (t, J = 6.9 Hz, 1H), 3.77-3.74 (m, 5H), 3.67- 3.63 (m, 1H), 3.33 (s, 3H), 3.07-3.04 (m, 4H), 2.94-2.84 (m, 1H), 2.80-2.72 (m, 2H), 2.40-2.30 (m, 1H), 2.25-2.19 (m, 1H), 1.77-1.61 (m, 2H), 1.28 (s, 3H), 1.21 (s, 3H), 1.02 (s, 3H). 1H NMR (300 MHz, DMSO-d6) δ 9.09 (s, 1H), 8.50 (d, J = 8.4 Hz, 1H), 7.98 (s, 1H), 7.68 (d, J = 8.4 Hz, 1H), 7.60 (d, J = 9.0 Hz, 2H), 6.93 (d, J = 9.0 Hz, 2H), 4.74-4.69 (m, 1H), 3.96 (t, J = 6.9 Hz, 1H), 3.80-3.64 (m, 6H), 3.33 (s, 3H), 3.07-3.04 (m, 4H), 2.92-2.71 (m, 3H), 2.39-2.28 (m, 1H), 2.21-2.15 (m, 1H), 1.82-1.75 (m, 1H), 1.66-1.58 (m, 1H), 1.28 (s, 3H), 1.21 (s, 3H), 1.02 (s, 3H). | LCMS (ES, m/z): 542.30 [M + H]+, Rt 0.633 min. LCMS (ES, m/z): 542.35 [M + H]+, Rt 0.600 min. |
| 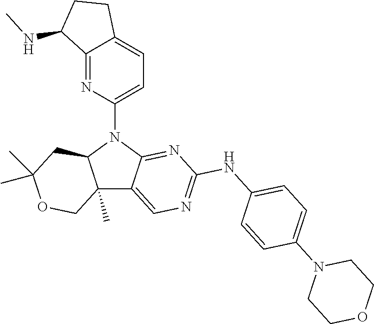 [SMILES: CN[C@H]1CCc2ccc(N3c4nc(Nc5ccc(N6CCOCC6)cc5)ncc4[C@@]4(C)COC(C)(C)C[C@@H]34)nc21] | | | |
| 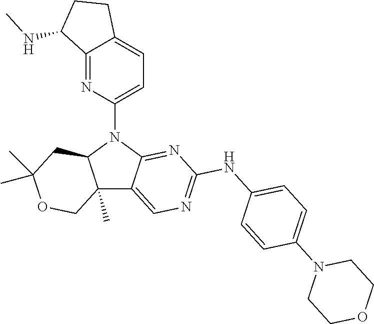 [SMILES: CN[C@@H]1CCc2ccc(N3c4nc(Nc5ccc(N6CCOCC6)cc5)ncc4[C@@]4(C)COC(C)(C)C[C@@H]34)nc21] | 553, 554 | 1H NMR (300 MHz, DMSO-d6) δ 9.09 (s, 1H), 8.49 (d, J = 8.4 Hz, 1H), 7.97 (s, 1H), 7.67-7.57 (m, 3H), 6.92 (d, J = 9.0 Hz, 2H), 4.73-4.68 (m, 1H), 3.96-3.91 (m, 1H), 3.79-3.74 (m, 5H), 3.68-3.64 (m, 1H), 3.07-3.04 (m, 4H), 2.95-2.70 (m, 2H), 2.49 (s, 3H), 2.39-2.15 (m, 2H), 1.84-1.58 (m, 2H), 1.28 (s, 3H), 1.21 (s, 3H), 1.01 (s, 3H). 1H NMR (300 MHz, DMSO-d6) δ 9.08 (s, 1H), 8.45 (d, J = 8.4 Hz, 1H), 7.97 (s, 1H), 7.67-7.57 (m, 3H), 6.92 (d, J = 9.0 Hz, 2H), 4.69-4.65 (m, 1H), 3.95-3.90 (m, 1H), 3.77-3.63 (m, 6H), 3.06-3.03 (m, 4H), 2.95-2.71 (m, 2H), 2.53 (s, 3H), 2.37-2.18 (m, 3H), 1.77-1.61 (m, 2H), 1.28 (s, 3H), 1.20 (s, 3H), 1.01 (s, 3H). | LCMS (ES, m/z): 542.35 [M + H]+, Rt 0.592 min. LCMS (ES, m/z): 542.35 [M + H]+, Rt 0.625 min. |
| 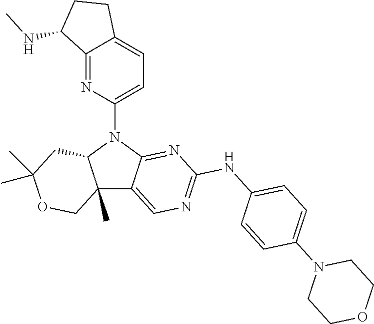 [SMILES: CN[C@@H]1CCc2ccc(N3c4nc(Nc5ccc(N6CCOCC6)cc5)ncc4[C@]4(C)COC(C)(C)C[C@H]34)nc21] | | | |
| 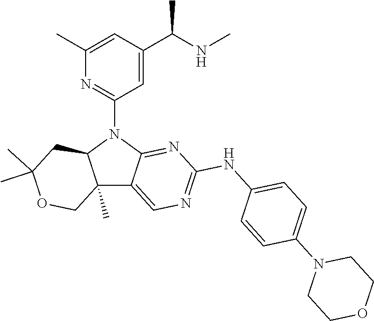 [SMILES: CN[C@H](C)c1cc(C)nc(N2c3nc(Nc4ccc(N5CCOCC5)cc4)ncc3[C@@]3(C)COC(C)(C)C[C@@H]23)c1] | 557, 558 | 1H NMR (400 MHz, DMSO-d6) δ 10.70 (s, 1H), 9.60 (s, 1H), 9.25 (s, 1H), 8.06 (br, 2H), 7.35 (d, J = 8.7 Hz, 2H), 7.26 (s, 1H), 7.04 (d, J = 8.7 Hz, 2H), 4.86-4.79 (m, 1H), 4.08-4.03 (m, 1H), 3.86-3.63 (m, 7H), 3.18-3.11 (m, 4H), 2.47-2.40 (m, 3H), 2.25-2.16 (m, 1H), 1.73-1.63 (m, 1H), 1.45 (s, 3H), 1.32 (s, 3H), 1.20 (s, 3H), 1.00 (s, 3H). 1H NMR (400 MHz, DMSO-d6) δ 10.76 (s, 1H), 9.62 (s, 1H), 9.28 (s, 1H), 8.22- 7.93 (m, 2H), 7.36 (d, J = 8.4 Hz, 2H), 7.33-7.26 (m, 1H), 7.22-7.00 (m, 3H), 4.89-4.81 (m, 1H), 4.09-4.04 (m, 1H), 3.82-3.74 (m, 6H), 3.69-3.62 (m, 1H), 3.20-3.11 (m, 4H), 2.49-2.43 (m, 3H), 2.19-2.06 (m, 1H), 1.83-1.73 (m, 1H), 1.46 (s, 3H), 1.33 (s, 3H), 1.19 (s, 3H), 0.98 (s, 3H). | LCMS (ES, m/z): 544.35 [M + H]+, Rt 0.658 min. LCMS (ES, m/z): 544.35 [M + H]+, Rt 0.667 min. |
| 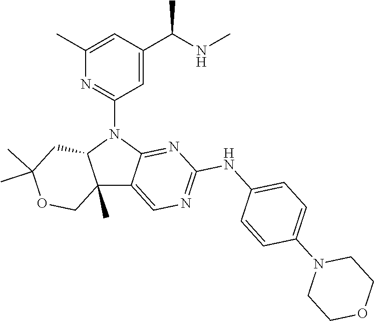 [SMILES: CN[C@H](C)c1cc(C)nc(N2c3nc(Nc4ccc(N5CCOCC5)cc4)ncc3[C@]3(C)COC(C)(C)C[C@H]23)c1] | | | |
| | 559, | 1H NMR (300 MHz, DMSO-d6) δ 9.02 (s, | LCMS (ES, |
| | 560 | 1H), 8.24 (s, 1H), 7.97 (s, 1H), 7.51 (d, J = | m/z): 544.35 |
| | | 8.7 Hz, 2H), 6.92-6.89 (m, 3H), 4.69- | [M + H]+, Rt |
| | | 4.64 (m, 1H), 3.80-3.74 (m, 5H), 3.67- | 0.658 min. |
| | | 3.62 (m, 1H), 3.54-3.52 (m, 1H), 3.06- | LCMS (ES, |
| | | 3.03 (m, 5H), 2.41 (s, 3H), 2.29-2.12 (m, | m/z): 544.35 |
| | | 1H), 2.08 (s, 3H), 1.60-1.57 (m, 1H), | [M + H]+, Rt |
| | | 1.25-1.22 (m, 9H), 1.02 (s, 3H). | 0.658 min. |
| | | 1H NMR (300 MHz, DMSO-d6) δ 9.02 (s, | |
| | | 1H), 8.23 (s, 1H), 7.97 (s, 1H), 7.51 (d, J = | |
| | | 9.0 Hz, 2H), 6.92-6.89 (m, 3H), 4.69- | |
| | | 4.64 (m, 1H), 3.80-3.74 (m, 5H), 3.67- | |
| | | 3.63 (m, 1H), 3.55-3.49 (m, 1H), 3.06- | |
| | | 3.03 (m, 5H), 2.41 (s, 3H), 2.28-2.12 (m, | |
| | | 1H), 2.08 (s, 3H), 1.60-1.57 (m, 1H), | |
| | | 1.33-1.16 (m, 9H), 1.02 (s, 3H). | |
| 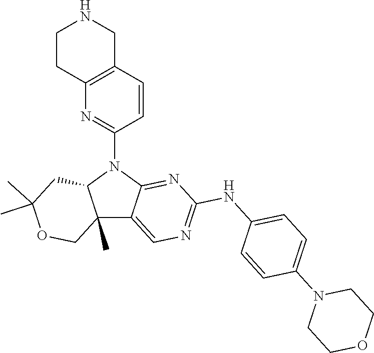 [SMILES: CC1(C)C[C@@H]2N(c3ccc4c(n3)CCNC4)c3nc(Nc4ccc(N5CCOCC5)cc4)ncc3[C@]2(C)CO1] | 561, 562 | 1H NMR (400 MHz, DMSO-d6) δ 9.04 (s, 1H), 8.35 (d, J = 8.4 Hz, 1H), 7.96 (s, 1H), 7.57 (d, J = 8.8 Hz, 2H), 7.44 (d, J = 8.8 Hz, 1H), 6.90 (d, J = 8.8 Hz, 2H), 4.66-4.63 (m, 1H), 3.82 (s, 2H), 3.76- 3.72 (m, 5H), 3.64-3.61 (m, 1H), 3.05- 3.00 (m, 6H), 2.72-2.67 (m, 3H), 2.17- 2.13 (m, 1H), 1.64-1.61 (m, 1H), 1.26 (s, 3H), 1.20 (s, 3H), 1.00 (s, 3H). 1H NMR (400 MHz, DMSO-d6) δ 9.04 (s, 1H), 8.35 (d, J = 8.8 Hz, 1H), 7.95 (s, 1H), 7.57 (d, J = 9.2 Hz, 2H), 7.44 (d, J = 8.4 Hz, 1H), 6.89 (d, J = 9.2 Hz, 2H), 4.66-4.63 (m, 1H), 3.82 (s, 2H), 3.76- 3.72 (m, 5H), 3.64-3.61 (m, 1H), 3.05- 3.00 (m, 6H), 2.71-2.67 (m, 3H), 2.17- 2.13 (m, 1H), 1.64-1.63 (m, 1H), 1.26 (s, 3H), 1.20 (s, 3H), 1.00 (s, 3H). | LCMS (ES, m/z): 528.30 [M + H]+, Rt 0.898 min. LCMS (ES, m/z): 528.30 [M + H]+, Rt 0.898 min. |
| 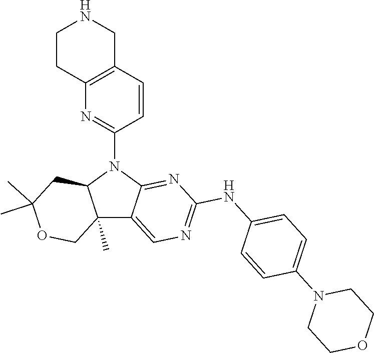 [SMILES: CC1(C)C[C@H]2N(c3ccc4c(n3)CCNC4)c3nc(Nc4ccc(N5CCOCC5)cc4)ncc3[C@@]2(C)CO1] | | | |
| 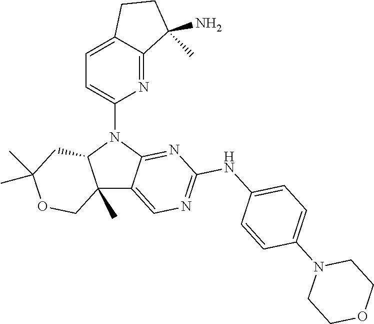 [SMILES: CC1(C)C[C@@H]2N(c3ccc4c(n3)[C@](C)(N)CC4)c3nc(Nc4ccc(N5CCOCC5)cc4)ncc3[C@]2(C)CO1] | 563, 564 | 1H NMR (300 MHz, DMSO-d6) δ 9.08 (s, 1H), 8.42 (d, J = 8.1 Hz, 1H), 7.96 (s, 1H), 7.64-7.57 (m, 3H), 6.90 (d, J = 9.0 Hz, 2H), 4.70-4.66 (m, 1H), 3.77-3.63 (m, 6H), 3.06-3.03 (m, 4H), 2.85-2.73 (m, 2H), 2.33-2.28 (m, 1H), 2.07-1.88 (m, 4H), 1.67-1.59 (m, 1H), 1.30-1.24 (m, 9H), 1.03 (s, 3H). 1H NMR (300 MHz, DMSO-d6) δ 9.08 (s, 1H), 8.44 (d, J = 8.4 Hz, 1H), 7.95 (s, 1H), 7.64-7.57 (m, 3H), 6.90 (d, J = 9.0 Hz, 2H), 4.76-4.73 (m, 1H), 3.77-3.64 (m, 6H), 3.06-3.03 (m, 4H), 2.86-2.80 (m, 1H), 2.76-2.73 (m, 1H), 2.28-2.19 (m, 1H), 2.05-1.97 (m, 2H), 1.81 (s, 2H), 1.65-1.57 (m, 1H), 1.32 (s, 3H), 1.30 (s, 3H), 1.25 (s, 3H), 1.02 (s, 3H). | LCMS (ES, m/z): 542.30 [M + H]+, Rt 0.583 min. LCMS (ES, m/z): 542.30 [M + H]+, Rt 0.625 min. |
| 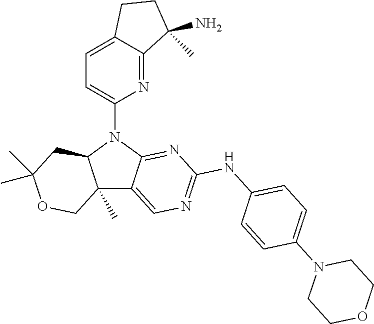 [SMILES: CC1(C)C[C@H]2N(c3ccc4c(n3)[C@](C)(N)CC4)c3nc(Nc4ccc(N5CCOCC5)cc4)ncc3[C@@]2(C)CO1] | | | |
| 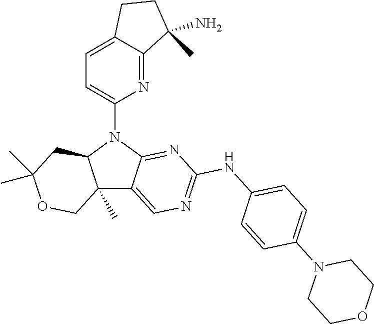 [SMILES: CC1(C)C[C@H]2N(c3ccc4c(n3)[C@@](C)(N)CC4)c3nc(Nc4ccc(N5CCOCC5)cc4)ncc3[C@@]2(C)CO1] | 565, 566 | 1H NMR (300 MHz, DMSO-d6) δ 9.08 (s, 1H), 8.42 (d, J = 8.4 Hz, 1H), 7.95 (s, 1H), 7.63-7.57 (m, 3H), 6.90 (d, J = 9.3 Hz, 2H), 4.70-4.66 (m, 1H), 3.77-63 (m, 6H), 3.06-3.03 (m, 4H), 2.85-2.73 (m, 2H), 2.33-2.28 (m, 1H), 2.07-1.87 (m, 4H), 1.67-1.59 (m, 1H), 1.30-1.24 (m, 9H), 1.03 (s, 3H). 1H NMR (300 MHz, DMSO-d6) δ 9.08 (s, 1H), 8.44 (d, J = 8.1 Hz, 1H), 7.96 (s, 1H), 7.64-7.57 (m, 3H), 6.90 (d, J = 9.0 Hz, 2H), 4.77-4.72 (m, 1H), 3.77-3.64 (m, 6H), 3.06-3.03 (m, 4H), 2.88-2.84 (m, 1H), 2.78-2.71 (m, 1H), 2.28-2.19 (m, 2H), 2.07-1.96 (m, 3H), 1.65-1.57 (m, 1H), 1.30-1.24 (m, 9H), 1.02 (s, 3H). | LCMS (ES, m/z): 542.30 [M + H]+, Rt 0.583 min. LCMS (ES, m/z): 542.30 [M + H]+, Rt 0.633 min. |
| 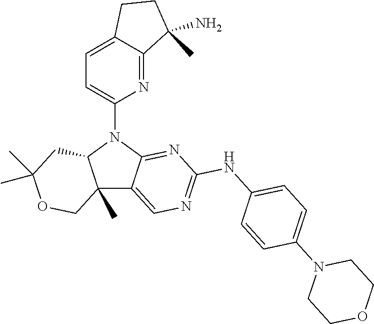 [SMILES: CC1(C)C[C@@H]2N(c3ccc4c(n3)[C@@](C)(N)CC4)c3nc(Nc4ccc(N5CCOCC5)cc4)ncc3[C@]2(C)CO1] | | | |
| 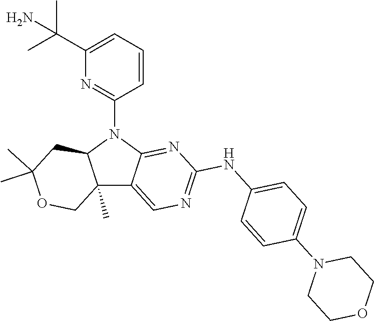 [SMILES: CC1(C)C[C@H]2N(c3cccc(C(C)(C)N)n3)c3nc(Nc4ccc(N5CCOCC5)cc4)ncc3[C@@]2(C)CO1] | 567 568 | 1H NMR (300 MHz, DMSO-d6) δ 9.10 (s, 1H), 8.49 (d, J = 8.4 Hz, 1H), 7.98 (s, 1H), 7.74 (t, J = 7.8 Hz, 1H), 7.60 (d, J = 8.4 Hz, 2H), 7.28 (d, J = 7.5 Hz, 1H), 6.93 (d, J = 9.0 Hz, 2H), 4.68-4.66 (m, 1H), 3.81-3.66 (m, 6H), 3.07-3.04 (m, 4H), 2.28-2.07 (m, 2H), 1.63-1.56 (m, 2H), 1.41 (s, 6H), 1.31 (s, 3H), 1.26 (s, 3H), 1.05 (s, 3H). 1H NMR (300 MHz, DMSO-d6) δ 9.10 (s, 1H), 8.49-8.47 (m, 1H), 7.98 (s, 1H), 7.74 (t, J = 7.5 Hz, 1H), 7.60 (d, J = 9.0 Hz, 2H), 7.28 (d, J = 7.5 Hz, 1H), 6.93 (d, J = 8.7 Hz, 2H), 4.69-4.64 (m, 1H), 3.80- 3.65 (m, 6H), 3.07-3.04 (m, 4H), 2.31- 2.07 (m, 2H), 1.63-1.55 (m, 2H), 1.41 (s, 6H), 1.31 (s, 3H), 1.26 (s, 3H), 1.05 (s, 3H). | LCMS (ES, m/z): 530.30 [M + H]+, Rt 0.600 min. LCMS (ES, m/z): 530.30 [M + H]+, Rt 0.592 min. |
| 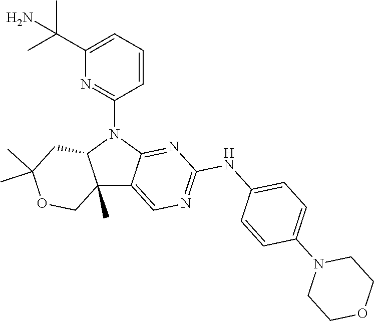 [SMILES: CC1(C)C[C@@H]2N(c3cccc(C(C)(C)N)n3)c3nc(Nc4ccc(N5CCOCC5)cc4)ncc3[C@]2(C)CO1] | | | |
| 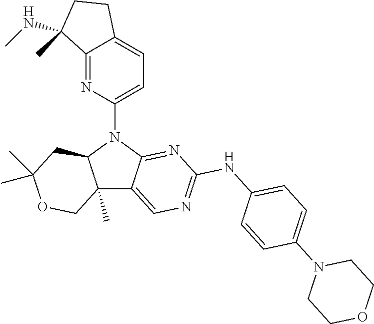 [SMILES: CN[C@]1(C)CCc2ccc(N3c4nc(Nc5ccc(N6CCOCC6)cc5)ncc4[C@@]4(C)COC(C)(C)C[C@@H]34)nc21] | 569, 570 | 1H NMR (300 MHz, DMSO-d6) δ 9.09 (s, 1H), 8.46 (d, J = 8.7 Hz, 1H), 7.96 (s, 1H), 7.66-7.57 (m, 3H), 6.91(d, J = 8.7 Hz, 2H), 4.70-4.66 (m, 1H), 3.76-3.64 (m, 6H), 3.07-3.04 (m, 4H), 2.94-2.84 (m, 1H), 2.79-2.69 (m, 1H), 2.29-2.09 (m, 5H), 1.99-1.90 (m, 1H), 1.69-1.61 (m, 1H), 1.35 (s, 3H), 1.30 (s, 3H), 1.23 (s, 3H), 1.02 (s, 3H). 1H NMR (300 MHz, DMSO-d6) δ 9.09 (s, 1H), 8.45 (d, J = 8.7 Hz, 1H), 7.97 (s, 1H), 7.68-7.57 (m, 3H), 6.91 (d, J = 8.4 Hz, 2H), 4.70-4.66 (m, 1H), 3.77-3.74 (m, 5H), 3.67-3.63 (m, 1H), 3.05 (t, J = 4.8 Hz, 4H), 2.92-2.75 (m, 3H), 2.24- 2.15 (m, 5H), 2.00-1.91 (m, 1H), 1.73- 1.66 (m, 1H), 1.36 (s, 3H), 1.30 (s, 3H), 1.22 (s, 3H), 1.01 (s, 3H). | LCMS (ES, m/z): 556.30 [M + H]+, Rt 0.642 min. LCMS (ES, m/z): 556.30 [M + H]+, Rt 0.617 min. |
| 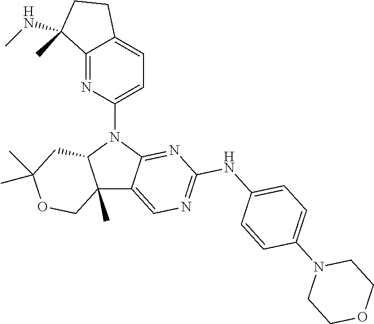 [SMILES: CN[C@]1(C)CCc2ccc(N3c4nc(Nc5ccc(N6CCOCC6)cc5)ncc4[C@]4(C)COC(C)(C)C[C@H]34)nc21] | | | |
| 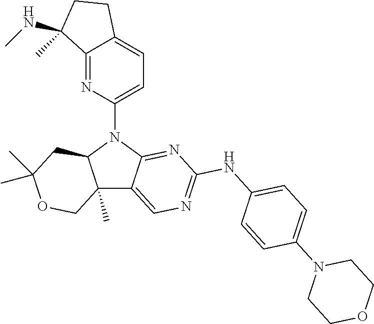 [SMILES: CN[C@@]1(C)CCc2ccc(N3c4nc(Nc5ccc(N6CCOCC6)cc5)ncc4[C@@]4(C)COC(C)(C)C[C@@H]34)nc21] | 571 | 1H NMR (300 MHz, DMSO-d6) δ 9.10 (s, 1H), 8.47 (d, J = 8.4 Hz, 1H), 7.98 (s, 1H), 7.68 (d, J = 8.7 Hz, 1H), 7.58 (d, J = 8.4 Hz, 2H), 6.93-6.90 (m, 2H), 4.71- 4.66 (m, 1H), 3.77-3.74 (m, 5H), 3.68- 3.64 (m, 1H), 3.07-3.04 (m, 4H), 2.96- 2.74 (m, 2H), 2.28-2.17 (m, 6H), 2.03- 1.95 (m, 1H), 1.73-1.66 (m, 1H), 1.39 (s, 3H), 1.30 (s, 3H), 1.22 (s, 3H), 1.01 (s, 3H). | LCMS (ES, m/z): 556.30 [M + H]+, Rt 0.567 min. |
| 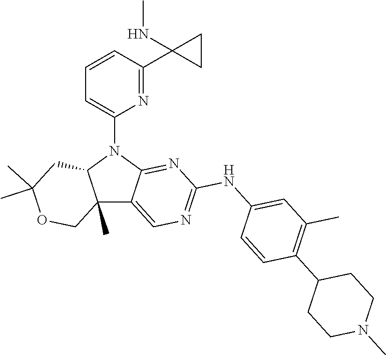 | 574, 575 | 1H NMR (400 MHz, DMSO-d6) δ 9.52- 9.48 (br, 1H), 9.32 (s, 1H), 8.59 (d, J = 8.4 Hz, 1H), 8.08 (s, 1H), 7.81 (t, J = 8.0 Hz, 1H), 7.59 (s, 1H), 7.48 (d, J = 8.0 Hz, 1H), 7.07-6.99 (m, 2H), 4.86-4.85 (m, 1H), 3.85-3.82 (m, 1H), 3.70-3.67 (m, 1H), 3.53-3.50 (m, 2H), 3.15-3.09 (m, 2H), 2.93-2.88 (m, 1H), 2.82 (s, 3H), 2.71-2.67 (m, 3H), 2.31 (s, 3H), 2.16- 2.11 (m, 1H), 1.92-1.81 (m, 4H), 1.57- 1.49 (m, 4H), 1.34-1.25 (m, 7H), 1.04 (s, 3H) 1H NMR (400 MHz, DMSO-d6) δ 9.19 (s, 1H), 8.41 (d, J = 8.4 Hz, 1H), 7.98 (s, 1H), 7.71 (d, J = 8.0 Hz, 1H), 7.56 (s, 1H), 7.42 (d, J = 8.4 Hz, 1H), 7.30 (d, J = 7.6 Hz, 1H), 7.10 (d, J = 8.4 Hz, 1H), 4.55-4.52 (m, 1H), 3.78-3.75 (m, 1H), 3.67-3.64 (m, 1H), 2.92-2.90 (m, 3H), 2.33-2.23 (m, 9H), 2.14-2.04 (m, 3H), 1.67-1.65 (m, 4H), 1.52-1.51 (m, 1H), 1.33-1.23 (m, 7H), 1.11-0.93 (m, 6H). | LCMS (ES, m/z): 568.40 [M + H]+, Rt 0.542 min. LCMS (ES, m/z): 568.35 [M + H]+, Rt 0.542 min. |
| 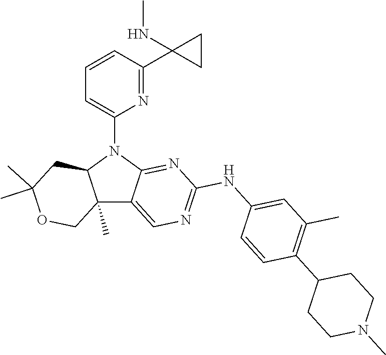 | | | |
| 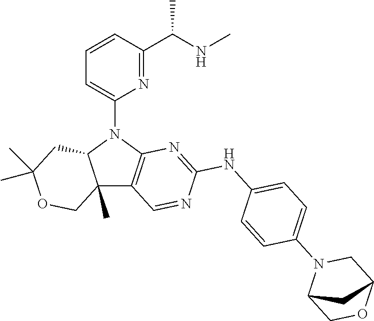 [SMILES: CN[C@@H](C)c1cccc(N2c3nc(Nc4ccc(N5C[C@H]6C[C@@H]5CO6)cc4)ncc3[C@]3(C)COC(C)(C)C[C@H]23)n1] | 578, 579 | 1H NMR (300 MHz, DMSO-d6) δ 8.96 (s, 1H), 8.48 (d, J = 8.4 Hz, 1H), 7.96 (s, 1H), 7.73 (t, J = 7.8 Hz, 1H), 7.48 (d, J = 8.4 Hz, 2H), 7.05 (d, J = 7.5 Hz, 1H), 6.60 (d, J = 8.7 Hz, 2H), 4.72-4.44 (m, 3H), 3.80-3.65 (m, 4H), 3.65-3.55 (m, 1H), 3.51 (d, J = 9.3 Hz, 1H), 2.94 (d, J = 9.3 Hz, 1H), 2.30-2.12 (m, 4H), 1.94 (d, J = 9.6 Hz, 1H), 1.84 (d, J = 9.6 Hz, 1H), 1.71-1.57 (m, 1H), 1.32-1.20 (m, 9H), 1.03 (s, 3H). 1H NMR (300 MHz, DMSO-d6) δ 8.96 (s, 1H), 8.49 (d, J = 8.4 Hz, 1H), 7.96 (s, 1H), 7.72 (t, J = 7.8 Hz, 1H), 7.49 (d, J = 8.4 Hz, 2H), 7.02 (d, J = 7.5 Hz, 1H), 6.60 (d, J = 8.7 Hz, 2H), 4.73-4.47 (m, 3H), 3.80-3.63 (m, 4H), 3.63-3.45 (m, 2H), 2.94 (d, J = 9.3 Hz, 1H), 2.30-2.14 (m, 4H), 1.94 (d, J = 9.6 Hz, 1H), 1.84 (d, J = 9.6 Hz, 1H), 1.70-1.57 (m, 1H), 1.32- | LCMS (ES, m/z): 542.35 [M + H]+, Rt 0.608 min. LCMS (ES, m/z): 542.35 [M + H]+, Rt 0.625 min. |
| | | 1.26 (m, 6H), 1.24 (s, 3H), 1.03 (s, 3H). | |
| 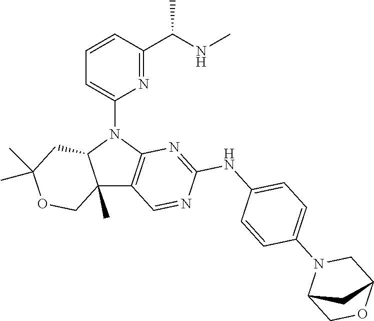 [SMILES: CN[C@@H](C)c1cccc(N2c3nc(Nc4ccc(N5C[C@H]6C[C@@H]5CO6)cc4)ncc3[C@]3(C)COC(C)(C)C[C@H]23)n1] | | | |
| 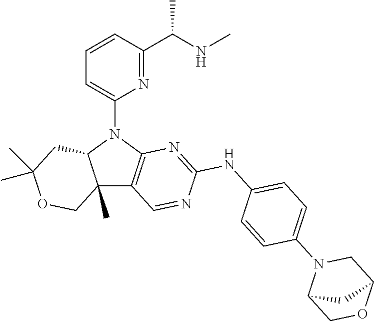 [SMILES: CN[C@@H](C)c1cccc(N2c3nc(Nc4ccc(N5C[C@@H]6C[C@H]5CO6)cc4)ncc3[C@]3(C)COC(C)(C)C[C@H]23)n1] | 580, 581 | 1H NMR (300 MHz, DMSO-d6) δ 8.96 (s, 1H), 8.49 (d, J = 8.4 Hz, 1H), 7.96 (s, 1H), 7.72 (t, J = 7.8 Hz, 1H), 7.49 (d, J = 8.7 Hz, 2H), 7.06-7.01 (m, 1H), 6.60 (d, J = 9.0 Hz , 2H), 4.69-4.60 (m, 2H), 4.50 (s, 1H), 3.77-3.68 (m, 4H), 3.64-3.57 (m, 1H), 3.50 (d, J = 9.3 Hz, 1H), 2.94 (d, J = 9.3 Hz, 1H), 2.22 (s, 3H), 2.19-2.11 (s, 1H), 1.99-1.79 (m, 2H), 1.70-1.54 (m, 1H), 1.28 (d, J = 6.8 Hz, 6H), 1.24 (s, 3H), 1.03 (s, 3H). 1H NMR (300 MHz, DMSO-d6) δ 8.96 (s, 1H), 8.49 (d, J = 7.8 Hz, 1H), 7.96 (d, J = 1.8 Hz, 1H), 7.72 (t, J = 8.0 Hz, 1H), 7.48 (d, J = 8.4 Hz, 2H), 7.02 (d, J = 7.5 Hz, 1H), 6.60 (d, J = 8.7 Hz, 2H), 4.70-4.60 (m, 2H), 4.51 (s, 1H), 3.77-3.68 (m, 4H), 3.64-3.49 (m, 2H), 2.94 (d, J = 9.1 Hz, 1H), 2.29-2.24 (m, 1H), 2.19 (s, 3H), 1.97-1.81 (m, 2H), 1.70-1.55 (m, 1H), | LCMS (ES, m/z): 542.35 [M + H]+, Rt 0.608 min. LCMS (ES, m/z): 542.35 [M + H]+, Rt 0.625 min. |
| | | 1.34-1.20 (m, 9H), 1.03 (s, 3H). | |
| 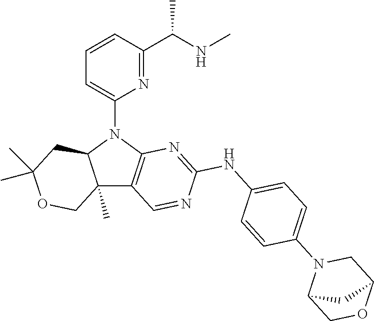 [SMILES: CN[C@@H](C)c1cccc(N2c3nc(Nc4ccc(N5C[C@@H]6C[C@H]5CO6)cc4)ncc3[C@@]3(C)COC(C)(C)C[C@@H]23)n1] | | | |
| 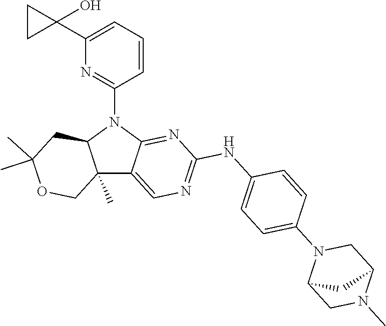 | 644, 645 | 1H NMR (400 MHz, DMSO-d6) δ 8.92 (s, 1H), 8.43 (d, J = 8.4 Hz, 1H), 7.91 (s, 1H), 7.73 (t, J = 8 Hz, 1H), 7.44-7.32 (m, 3H), 6.55 (d, J = 9.2 Hz, 2H), 6.11 (s, 1H), 4.48-4.44 (m, 1H), 4.24 (s, 1H), 3.79-3.61 (m, 2H), 3.42-3.38 (m, 1H), 3.33-3.28 (m, 2H), 3.13 (d, J = 9.2 Hz, 1H), 2.77-2.75 (m, 1H), 2.25 (s, 3H), 2.13-2.08 (m, 1H), 1.86 (d, J = 8.8 Hz, 1H), 1.77 (d, J = 8.8 Hz, 1H), 1.53-1.47 (m, 1H), 1.32-1.24 (m, 4H), 1.20-1.15 (m, 4H), 1.09-1.03 (m, 5H). 1H NMR (400 MHz, DMSO-d6) δ 8.92 (s, 1H), 8.43 (d, J = 8.0 Hz, 1H), 7.91 (s, 1H), 7.73 (t, J = 8.0 Hz, 1H), 7.44-7.32 (m, 3H), 6.55 (d, J = 9.2 Hz, 2H), 6.11 (s, 1H), 4.48-4.44 (m, 1H), 4.24 (s, 1H), 3.79-3.62 (m, 2H), 3.41-3.38 (m, 1H), 3.33-3.30 (m, 2H), 3.13 (d, J = 9.2 Hz, 1H), 2.77-2.75 (m, 1H), 2.24 (s, 3H), | LCMS (ES, m/z): 554 [M + H]+. Rt 0.626 min. LCMS (ES, m/z): 554 [M + H]+. Rt 0.622 min. |
| | | 2.13-2.08 (m, 1H), 1.86 (d, J = 8.8 Hz, | |
| | | 1H), 1.77 (d, J = 8.8 Hz, 1H), 1.53-1.47 | |
| | | (m, 1H), 1.32-1.24 (m, 4H), 1.20-1.15 | |
| | | (m, 4H), 1.12-1.08 (m, 2H), 1.04 (s, 3H). | |
| 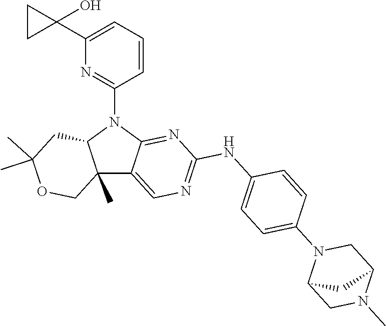 | | | |
| 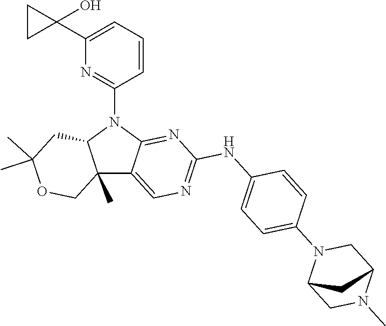 | 646, 647 | 1H NMR (300 MHz, DMSO-d6) δ 8.91 (s, 1H), 8.41 (d, J = 8.4 Hz, 1H), 7.91 (s, 1H), 7.70 (t, J = 8.0 Hz, 1H), 7.43 (d, J = 8.7 Hz, 2H), 7.33 (d, J = 7.2 Hz, 1H), 6.54 (d, J = 9.0 Hz, 2H), 6.11 (s, 1H), 4.48- 4.43 (m, 1H), 4.24 (s, 1H), 3.75-3.61 (m, 2H), 3.41-3.32 (m, 1H), 3.13-3.10 (m, 1H), 2.78-2.75 (m, 1H), 2.53-2.51 (m, 2H), 2.26 (s, 3H), 2.14-2.08 (m, 1H), 1.87- 1.74 (m, 2H), 1.53-1.45(m, 1H), 1.31-1.11 (m, 8H), 1.08-1.04 (m, 2H), 1.03 (s, 3H). 1H NMR (300 MHz, DMSO-d6) δ 8.91 (s, 1H), 8.41 (d, J = 8.4 Hz, 1H), 7.91 (s, 1H), 7.70 (t, J = 8.0 Hz, 1H), 7.43 (d, J = 8.7 Hz, 2H), 7.33 (d, J = 7.2 Hz, 1H), 6.54 (d, J = 9.0 Hz, 2H), 6.11 (s, 1H), 4.48- 4.43 (m, 1H), 4.24 (s, 1H), 3.75-3.61 (m, 2H), 3.41-3.32 (m, 1H), 3.13-3.10 (m, 1H), 2.78-2.75 (m, 1H), 2.53-2.51 (m, | LCMS (ES, m/z): 554 [M + H]+, Rt 0.611 min. LCMS (ES, m/z): 554 [M + H]+, Rt 1.187 min. |
| | | 2H), 2.26 (s, 3H), 2.14-2.08 (m, 1H), | |
| | | 1.87- 1.74 (m, 2H), 1.53-1.45(m, 1H), | |
| | | 1.31-1.11 (m, 8H), 1.08-1.04 (m, 2H), | |
| | | 1.03 (s, 3H). | |
| 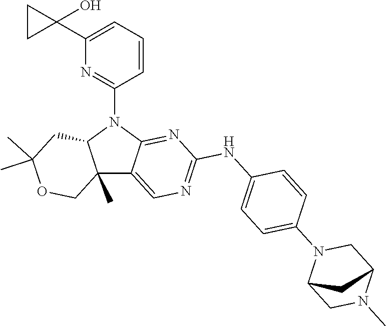 | | | |
| 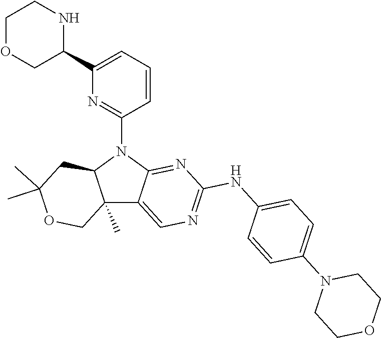 [SMILES: CC1(C)C[C@H]2N(c3cccc([C@H]4COCCN4)n3)c3nc(Nc4ccc(N5CCOCC5)cc4)ncc3[C@@]2(C)CO1] | 731, 732 | 1H NMR (300 MHz, DMSO-d6) δ 9.12 (s, 1H), 8.54 (d, J = 8.4 Hz, 1H), 7.99 (s, 1H), 7.77 (t, J = 8.1 Hz, 1H), 7.57 (d, J = 8.4 Hz, 2H), 7.13 (d, J = 7.5 Hz, 1H), 6.90 (d, J = 8.4 Hz, 2H), 4.80 - 4.63 (m, 1H), 4.01 - 3.97 (m, 1H), 3.86 - 3.64 (m, 8H), 3.40 - 3.10 (m, 3H), 3.06 - 3.03 (m, 4H), 2.90 - 2.88 (m, 2H), 2.21 - 2.17 (m, 1H), 1.61 - 1.53 (m, 1H), 1.28 (s, 3H), 1.24 (s, 3H), 1.03 (s, 3H). 1H NMR (300 MHz, DMSO-d6) δ 9.12 (s, 1H), 8.54 (d, J = 8.4 Hz, 1H), 8.00 (s, 1H), 7.77 (t, J = 7.9 Hz, 1H), 7.58 (d, J = 9.0 Hz, 2H), 7.11 (d, J = 7.2 Hz, 1H), 6.91 (d, J = 9.0 Hz, 2H), 4.69 - 4.65 (m, 1H), 4.01 - 3.97 (m, 1H), 3.87 - 3.83 (m, 1H), 3.89 - 3 .70 (m, 6H), 3.71 - 3.62 (m, 1H), 3.43 - 3.38 (m, 3H), 3.07 - 3.04 (m, 4H), 2.92 - 2.89 (m, 2H), 2.27 - 2.21 (m, 1H), 1.66 - 1.58 (m, 1H), 1.30 (s, 3H), 1.26 (s, 3H), 1.04 (s, 3H). | LCMS (ES, m/z): 558.30 [M + H]+, Rt 0.562 min. LCMS (ES, m/z): 558.25 [M + H]+, Rt 0.578 min. |
| 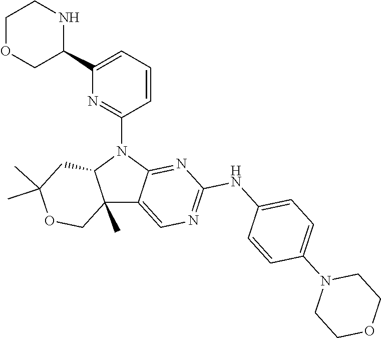 [SMILES: CC1(C)C[C@@H]2N(c3cccc([C@H]4COCCN4)n3)c3nc(Nc4ccc(N5CCOCC5)cc4)ncc3[C@]2(C)CO1] | | | |
| 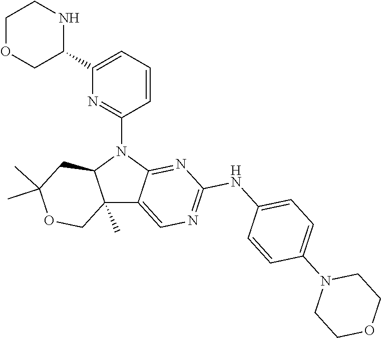 [SMILES: CC1(C)C[C@H]2N(c3cccc([C@@H]4COCCN4)n3)c3nc(Nc4ccc(N5CCOCC5)cc4)ncc3[C@@]2(C)CO1] | 733, 734 | 1H NMR (300 MHz, DMSO-d6) δ 9.12 (s, 1H), 8.56 (d, J = 8.4 Hz, 1H), 7.99 (s, 1H), 7.78 (t, J = 7.8 Hz, 1H), 7.59 (d, J = 8.7 Hz, 2H), 7.15 (d, J = 7.2 Hz, 1H), 6.93 (d, J = 8.7 Hz, 2H), 4.69-4.64 (m, 1H), 4.03-3.98 (m, 1H), 3.87-3.83 (m, 1H), 3.80-3.74 (m, 6H), 3.69-3.65 (m, 1H), 3.07-3.04 (m, 3H), 3.51-3.42 (m, 4H), 2.91-2.89 (m, 2H), 2.23-2.17 (m, 1H), 1.62-1.54 (m, 1H), 1.29 (s, 3H), 1.25 (s, 3H), 1.04 (s, 3H). 1H NMR (300 MHz, DMSO-d6) δ 9.12 (s, 1H), 8.55 (d, J = 8.7 Hz, 1H), 8.00 (s, 1H), 7.77 (t, J = 7.5 Hz, 1H), 7.59 (d, J = 8.7 Hz, 2H), 7.11 (d, J = 7.2 Hz, 1H), 6.91 (d, J = 8.7 Hz, 2H), 4.69-4.65 (m, 1H), 4.01-3.96 (m, 1H), 3.87-3.83 (m, 1H), 3.80-3.74 (m, 6H), 3.69-3.65 (m, 1H), 3.51-3.42 (m, 3H), 3.07-3.04 (m, 4H), 2.91-2.89 (m, 2H), 2.27-2.21 (m, 1H), 1.66-1.58 (m, 1H), 1.29 (m, 3H), 1.26 | LCMS (ES, m/z): 558.25 [M + H]+, Rt 0.553 min. LCMS (ES, m/z): 558.25 [M + H]+, Rt 0.578 min. |
| | | (m, 3H), 1.04 (s, 3H). | |
| 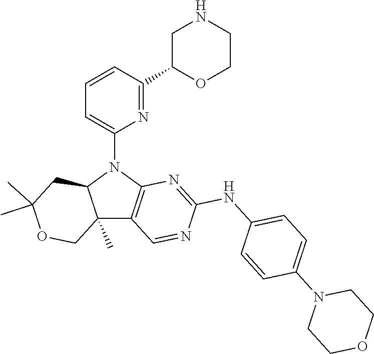 [SMILES: CC1(C)C[C@H]2N(c3cccc([C@@H]4CNCCO4)n3)c3nc(Nc4ccc(N5CCOCC5)cc4)ncc3[C@@]2(C)CO1] | 735, 736 | 1H NMR (400 MHz, DMSO-d6) δ 9.08 (s, 1H), 8.50 (d, J = 8.4 Hz, 1H), 7.98 (s, 1H), 7.78 (t, J = 8.0 Hz, 1H), 7.56 (d, J = 9.2 Hz, 2H), 7.06 (d, J = 7.2 Hz, 1H), 6.90 (d, J = 9.2 Hz, 2H), 4.60-4.59 (m, 1H), 4.42-4.39 (m, 1H), 3.91-3.76 (m, 1H), 3.78-3.71 (m, 5H), 3.69-3.61 (m, 2H), 3.20-3.15 (m, 2H), 3.05 (t, J = 4.8 Hz, 4H), 2.78-2.70 (m, 2H), 2.66-2.56 (m, 1H), 2.24-2.16 (m, 1H), 1.71-1.62 (m, 1H), 1.28 (s, 3H), 1.23 (s, 3H), 1.01 (s, 3H) 1H NMR (400 MHz, DMSO-d6) δ 9.09 (s, 1H), 8.51 (d, J = 8.4 Hz, 1H), 7.99 (s, 1H), 7.78 (t, J = 8.0 Hz, 1H), 7.56 (d, J = 9.2 Hz, 2H), 7.07 (d, J = 7.6 Hz, 1H), 6.91 (d, J = 9.2 Hz, 2H), 4.60-4.59 (m, 1H), 4.42-4.39 (m, 1H), 3.91-3.76 (m, 1H), 3.78-3.71 (m, 5H), 3.69-3.61 (m, 2H), 3.20-3.15 (m, 2H), 3.05 (t, J = 4.8 Hz, 4H), 2.78-2.70 (m, 2H), 2.66-2.56 (m, 1H), 2.24-2.16 (m, 1H), 1.71-1.62 (m, | LCMS (ES, m/z): 558.30 [M + H]+, Rt 0.578 min. LCMS (ES, m/z): 558.25 [M + H]+, Rt 0.570 min. |
| | | 1H), 1.28 (s, 3H), 1.23 (s, 3H), 1.02 (s, | |
| | | 3H). | |
| 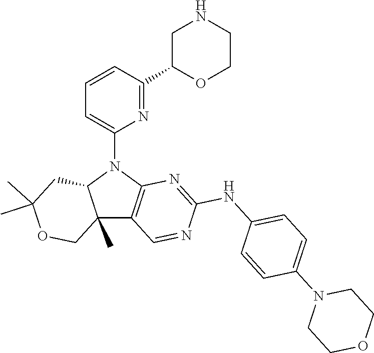 [SMILES: CC1(C)C[C@@H]2N(c3cccc([C@@H]4CNCCO4)n3)c3nc(Nc4ccc(N5CCOCC5)cc4)ncc3[C@]2(C)CO1] | | | |
| 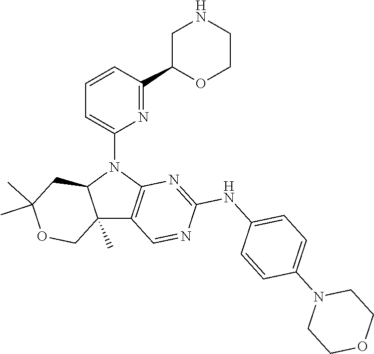 [SMILES: CC1(C)C[C@H]2N(c3cccc([C@H]4CNCCO4)n3)c3nc(Nc4ccc(N5CCOCC5)cc4)ncc3[C@@]2(C)CO1] | 737, 738 | 1H NMR (400 MHz, DMSO-d6) δ 9.09 (s, 1H), 8.54 (d, J = 8.4 Hz, 1H), 7.99 (s, 1H), 7.82-7.74 (m, 1H), 7.61-7.53 (m, 2H), 7.08 (d, J = 7.6 Hz, 1H), 6.95-6.87 (m, 2H), 4.65-4.61 (m, 1H), 4.42-4.39 (m, 1H), 3.94-3.90 (m, 1H), 3.77-3.74 (m, 5H), 3.67-3.61 (m, 2H), 3.28-3.19 (m, 2H), 3.08-3.01 (m, 4H), 2.80-2.66 (m, 2H), 2.62-2.55 (m, 1H), 2.18- 2.08 (m, 1H), 1.61-1.55 (m, 1H), 1.28 (s, 3H), 1.23 (s, 3H), 1.02 (s, 3H). 1H NMR (400 MHz, DMSO-d6) δ 9.09 (s, 1H), 8.54 (d, J = 8.4 Hz, 1H), 7.99 (s, 1H), 7.82-7.74 (m, 1H), 7.61-7.53 (m, 2H), 7.08 (d, J = 7.6 Hz, 1H), 6.95-6.87 (m, 2H), 4.65-4.61 (m, 1H), 4.42-4.39 (m, 1H), 3.94-3.90 (m, 1H), 3.77-3.74 (m, 5H), 3.67-3.61 (m, 2H), 3.28-3.19 (m, 2H), 3.08-3.01 (m, 4H), 2.80-2.66 (m, 2H), 2.62-2.55 (m, 1H), 2.18- 2.08 (m, 1H), 1.61-1.55 (m, 1H), 1.28 (s, 3H), 1.23 (s, 3H), 1.02 (s, 3H). | LCMS (ES, m/z): 558.25 [M + H]+, Rt 0.578 min. LCMS (ES, m/z): 558.25 [M + H]+, Rt 0.587 min. |
| 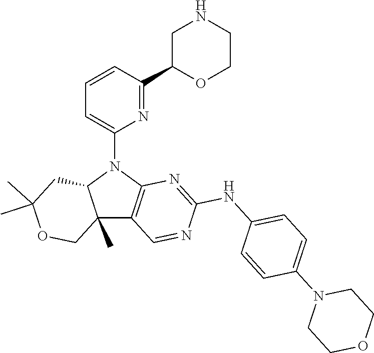 [SMILES: CC1(C)C[C@@H]2N(c3cccc([C@H]4CNCCO4)n3)c3nc(Nc4ccc(N5CCOCC5)cc4)ncc3[C@]2(C)CO1] | | | |

### Examples 111 and 112: (1R,9R)-8-[6-[[dimethyl(oxo)-sulfanylidene]amino]-2-pyridyl]-1,11,11-trimethyl-N-[4-(1-methyl-4-piperidyl)phenyl]-12-oxa-4,6,8-triazatricyclo[7.4.0.02,7]trideca-2,4,6-trien-5-amine and (1S,9S)-8-[6-[[dimethyl(oxo)-sulfanylidene]amino]-2-pyridyl]-1,11,11-trimethyl-N-[4-(1-methyl-4-piperidyl)phenyl]-12-oxa-4,6,8-triazatricyclo[7.4.0.02,7]trideca-2,4,6-trien-5-amine

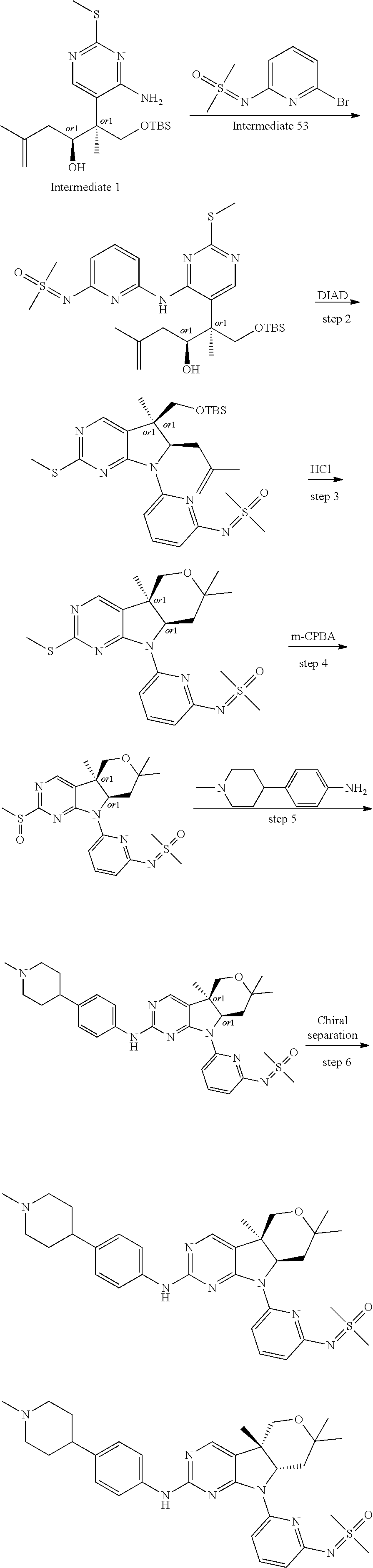

### Step 1. rel-(2S,3S)-1-[tert-Butyl(dimethyl)silyl]oxy-2-[4-[[6-[[dimethyl(oxo)-sulfanylidene]amino]-2-pyridyl]amino]-2-methylsulfanyl-pyrimidin-5-yl]-2,5-dimethyl-hex-5-en-3-ol

To a stirred solution of rel-(2S,3S)-2-(4-amino-2-(methylthio)pyrimidin-5-yl)-1-((tert-butyldimethylsilyl)oxy)-2,5-dimethylhex-5-en-3-ol (Intermediate 1, 600 mg, 1.51 mmol, 1 eq.) in dioxane (10 mL) was added (6-bromo-2-pyridyl)imino-dimethyl-oxo-sulfane (Intermediate 53, 563.85 mg, 2.26 mmol, 1.5 eq.), cesium carbonate (1.47 g, 4.53 mmolm, 3 eq.), BINAP-Pd-G2 (140.63 mg, 150.89 umol, 0.1 eq.) and BINAP (93.95 mg, 150.89 umol, 0.1 eq.). The resulting mixture was stirred for 2 h at 100° C. under the N₂ atmosphere. The reaction was monitored by LCMS. The mixture was allowed to cool down to room temperature. The solids were filtered and were washed with DCM (3×20 mL). The filtrate was concentrated under reduced pressure. The residue was purified by silica gel column chromatography (eluting with 1:1 EA/PE) to afford rel-(2S,3S)-1-[tert-butyl(dimethyl)silyl]oxy-2-[4-[[6-[[dimethyl(oxo)-sulfanylidene]amino]-2-pyridyl]amino]-2-methylsulfanyl-pyrimidin-5-yl]-2,5-dimethyl-hex-5-en-3-ol (680 mg, 1.20 mmol, 80% yield) as a yellow oil. LCMS (ES, m/z): 566 [M+H]⁺, Rt 1.058 min.

### Step 2. rel-((6-((5R,6R)-5-(((tert-butyldimethylsilyl)oxy)methyl)-5-methyl-6-(2-methylallyl)-2-(methylthio)-5,6-dihydro-7H-pyrrolo[2,3-d]pyrimidin-7-yl)pyridin-2-yl)imino)dimethyl-l6-sulfanone

To a solution of rel-(2S,3S)-1-[tert-butyl(dimethyl)silyl]oxy-2-[4-[[6-[[dimethyl(oxo)-sulfanylidene]amino]-2-pyridyl]amino]-2-methylsulfanyl-pyrimidin-5-yl]-2,5-dimethyl-hex-5-en-3-ol (735 mg, 1.30 mmol, 1 eq.) and triphenylphosphane (681.37 mg, 2.60 mmol, 2 eq.) in THF (10 mL) was added diisopropyl azodicarboxylate (525.30 mg, 2.60 mmol, 2 eq.) dropwise. The resulting mixture was stirred for 3 h at 50° C. under the N₂ atmosphere. The reaction was monitored by LCMS. The mixture was allowed to cool down to room temperature and was concentrated under reduced pressure. The residue was purified by silica gel chromatography (eluting with 1:10 MeOH/DCM) to afford rel-((6-((5R,6R)-5-(((tert-butyldimethylsilyl)oxy)methyl)-5-methyl-6-(2-methylallyl)-2-(methylthio)-5,6-dihydro-7H-pyrrolo[2,3-d]pyrimidin-7-yl)pyridin-2-yl)imino)dimethyl-l6-sulfanone (640 mg, 1.13 mmol, 87% yield) as a yellow oil. LCMS (ES, m/z): 548 [M+H]+, Rt 1.112 min.

### Step 3. rel-dimethyl((6-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)imino)-l6-sulfanone

To a stirred solution of rel-((6-((5R,6R)-5-(((tert-butyldimethylsilyl)oxy)methyl)-5-methyl-6-(2-methylallyl)-2-(methylthio)-5,6-dihydro-7H-pyrrolo[2,3-d]pyrimidin-7-yl)pyridin-2-yl)imino)dimethyl-l6-sulfanone (330 mg, 602.35 umol, 1 eq.) in dioxane (6 mL) was added HCl (4 M in dioxane, 2 mL) dropwise. The resulting mixture was stirred for 3 h at room temperature. The reaction was monitored by LCMS. The mixture was neutralized to pH=8 with saturated NaHCO₃. The resulting mixture was extracted with EA (3×50 mL). The combined organic layers were dried over anhydrous Na₂SO₄. After filtration, the filtrate was concentrated under reduced pressure. The residue was purified by silica gel column chromatography (eluting with 1:1 EA/PE) to afford rel-dimethyl((6-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)imino)-l6-sulfanone (164 mg, 379.48 umol, 63% yield) as a yellow oil. LCMS (ES, m/z): 434 [M+H]⁺, Rt 0.658 min.

### Step 4. rel-dimethyl((6-((4bR,8aR)-4b,7,7-trimethyl-2-(methylsulfinyl)-4b,7,8,8a-tetrahydropyrano[3′,4′:4,5]pyrrolo[2,3-d]pyrimidin-9(5H)-yl)pyridin-2-yl)imino)-l6-sulfanone

To a stirred mixture of rel-dimethyl((6-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)imino)-l6-sulfanone (90 mg, 207.57 umol, 1 eq.) in DCM (1 mL) was added m-CPBA (46.55 mg, 269.84 umol, 1.3 eq.) at 0° C. The resulting mixture was stirred for 2 h at room temperature. The reaction was monitored by LCMS. The mixture was concentrated to afford rel-dimethyl((6-((4bR,8aR)-4b,7,7-trimethyl-2-(methylsulfinyl)-4b,7,8,8a-tetrahydropyrano[3′,4′:4,5]pyrrolo[2,3-d]pyrimidin-9(5H)-yl)pyridin-2-yl)imino)-l6-sulfanone (90 mg, crude), which was used in the next step directly without further purification. LCMS (ES, m/z): 450 [M+H]+, Rt 0.520 min.

### Step 5. rel-dimethyl((6-((4bR,8aR)-4b,7,7-trimethyl-2-((4-(1-methylpiperidin-4-yl)phenyl)amino)-4b,7,8,8a-tetrahydropyrano[3′,4′:4,5]pyrrolo[2,3-d]pyrimidin-9(5H)-yl)pyridin-2-yl)imino)-l6-sulfanone

To a stirred mixture of rel-dimethyl((6-((4bR,8aR)-4b,7,7-trimethyl-2-(methylsulfinyl)-4b,7,8,8a-tetrahydropyrano[3′,4′:4,5]pyrrolo[2,3-d]pyrimidin-9(5H)-yl)pyridin-2-yl)imino)-l6-sulfanone (80 mg, 177.94 μmol, 1 eq.) and 4-(1-methyl-4-piperidyl)aniline (Intermediate 66, 67.72 mg, 355.88 μmol, 2 eq.) in toluene (1 mL) was added DIEA (114.77 mg, 889.70 μmol, 5 eq.) dropwise. The resulting mixture was stirred for 16 h at 100° C. The reaction was monitored by LC-MS. The mixture was allowed to cool down to room temperature and was concentrated under reduced pressure. The crude product was purified by reverse flash chromatography with the following conditions (Column: C18 Column, 40-60 nm, 40 g; Mobile Phase A: water (10 mmol/L NH₄HCO₃), Mobile Phase B: ACN (80% to 80% in 60 min); 254 nm) to afford rel-dimethyl((6-((4bR,8aR)-4b,7,7-trimethyl-2-((4-(1-methylpiperidin-4-yl)phenyl)amino)-4b,7,8,8a-tetrahydropyrano[3′,4′:4,5]pyrrolo[2,3-d]pyrimidin-9(5H)-yl)pyridin-2-yl)imino)-l6-sulfanone (85 mg, 147.63 μmol, 83% yield) as a yellow solid. LCMS (ES, m/z): 576 [M+H]⁺, Rt 0.530 min.

### Step 6. Separation of Enantiomers to Obtain (1R,9R)-8-[6-[[dimethyl(oxo)-sulfanylidene]amino]-2-pyridyl]-1,11,11-trimethyl-N-[4-(1-methyl-4-piperidyl)phenyl]-12-oxa-4,6,8-triazatricyclo[7.4.0.02,7]trideca-2,4,6-trien-5-amine and (1S,9S)-8-[6-[[dimethyl(oxo)-sulfanylidene]amino]-2-pyridyl]-1,11,11-trimethyl-N-[4-(1-methyl-4-piperidyl)phenyl]-12-oxa-4,6,8-triazatricyclo[7.4.0.02,7]trideca-2,4,6-trien-5-amine (Examples 111 and 112)

Racemic rel-dimethyl((6-((4bR,8aR)-4b,7,7-trimethyl-2-((4-(1-methylpiperidin-4-yl)phenyl)amino)-4b,7,8,8a-tetrahydropyrano[3′,4′:4,5]pyrrolo[2,3-d]pyrimidin-9(5H)-yl)pyridin-2-yl)imino)-l6-sulfanone (85 mg, 147.63 μmol, 1 eq.) was separated by chiral-HPLC with the following conditions (Column: Column: CHIRAL ART Cellulose-SC, 2*25 cm, 5 μm; Mobile Phase A: Hex (0.5% 2M NH₃-MeOH)-HPLC, Mobile Phase B: EtOH-HPLC; Flow rate: 20 mL/min; Gradient: 40% B to 40% B in 12 min; Wave Length: 220/254 nm; RT1(min): 8.57; RT2(min): 10.27; Sample Solvent: EtOH-HPLC; Injection Volume: 0.6 mL; Number Of Runs: 8) to afford (1R,9R)-8-[6-[[dimethyl(oxo)-sulfanylidene]amino]-2-pyridyl]-1,11,11-trimethyl-N-[4-(1-methyl-4-piperidyl)phenyl]-12-oxa-4,6,8-triazatricyclo[7.4.0.02,7]trideca-2,4,6-trien-5-amine (25.9 mg, 44.65 umol, 30% yield) as a white solid (the first eluting isomer) and (1S,9S)-8-[6-[[dimethyl(oxo)-sulfanylidene]amino]-2-pyridyl]-1,11,11-trimethyl-N-[4-(1-methyl-4-piperidyl)phenyl]-12-oxa-4,6,8-triazatricyclo[7.4.0.02,7]trideca-2,4,6-trien-5-amine (23.4 mg, 40.51 umol, 28% yield) as a white solid (the second eluting isomer).

Example 111: ¹H NMR (300 MHz, DMSO-d₆) δ 9.23 (s, 1H), 8.12 (d, J=8.4 Hz, 1H), 8.01 (s, 1H), 7.65 (d, J=8.1 Hz, 2H), 7.56 (t, J=7.8 Hz, 1H), 7.15 (d, J=8.4 Hz, 2H), 6.33 (d, J=8.1 Hz, 1H), 4.56-4.49 (m, 1H), 3.86-3.60 (m, 2H), 3.50 (s, 3H), 3.26 (s, 3H), 2.92-2.84 (m, 2H), 2.47-2.35 (m, 2H), 2.20 (s, 3H), 2.04-1.92 (m, 2H), 1.88-1.62 (m, 4H), 1.58-1.48 (m, 1H), 1.28 (s, 3H), 1.24 (s, 3H), 1.03 (s, 3H). LCMS (ES, m/z): 576 [M+H]⁺, Rt 0.558 min.

Example 112:: ¹H NMR (300 MHz, DMSO-d₆) δ 9.23 (s, 1H), 8.11 (d, J=8.4 Hz, 1H), 8.03 (s, 1H), 7.65 (d, J=8.7 Hz, 2H), 7.56 (t, J=8.1 Hz, 1H), 7.15 (d, J=8.7 Hz, 2H), 6.33 (d, J=7.5 Hz, 1H), 4.56-4.49 (m, 1H), 3.86-3.60 (m, 2H), 3.50 (s, 3H), 3.34 (s, 3H), 2.92-2.83 (m, 2H), 2.47-2.35 (m, 2H), 2.22 (s, 3H), 2.05-1.89 (m, 2H), 1.81-1.61 (m, 4H), 1.58-1.46 (m, 1H), 1.28 (s, 3H), 1.24 (s, 3H), 1.03 (s, 3H). LCMS (ES, m/z): 576 [M+H]⁺, Rt 0.567 min.

TABLE 14
The examples in the following table were synthesized according to the appropriate method as demonstrated in Examples 111-112.
Absolute stereochemistry of the examples in the following table was arbitrarily assigned.

| Structure | EX # | ¹H NMR | LCMS | Chiral conditions |
|---|---|---|---|---|
| 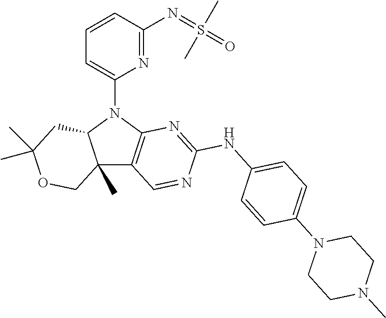 | 99, 100 | 1H NMR (300 MHz, DMSO-d6) δ 9.04 (s, 1H), 8.11 (d, J = 8.1 Hz, 1H), 7.98 (s, 1H), 7.60- 7.50 (m, 3H), 6.88 (d, J = 8.4 Hz, 2H), 6.31 (d, J = 8.1 Hz, 1H), 4.57-4.49 (m, 1H), 3.83-3.78 (m, 1H), 3.66-3.61 (m, 1H), 3.49 (s, 3H), 3.30 (s, 3H), 3.12-3.04 (m, 4H), 2.48-2.40 (m, 5H), 2.23 (s, 3H), 1.58-1.44 (m, 1H), 1.26 (s, 3H), 1.24 (s, 3H), 1.03 (s, 3H). | LCMS (ES, m/z): 577 [M + H]+, Rt 0.575 min. | Column: CHIRALPAK AD- H, 2*25 cm, 5 um; Mobile Phase A: Hex(0.5% 2 M NH3—MeOH)-- HPLC, Mobile Phase B: IPA-- HPLC; Flow rate: 20 mL/min; Gradient: 40 B to 40 B in 30 min; 220/254 nm; RT1: 19.9; RT2: 26.144 |
| 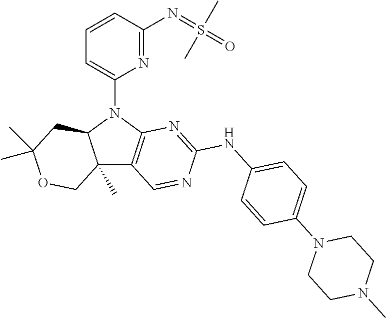 | | 1H NMR (300 MHz, DMSO-d6) δ 9.03 (s, 1H), 8.10 (d, J = 8.1 Hz, 1H), 7.96 (s, 1H), 7.59- 7.49 (m, 3H), 6.88 (d, J = 8.4 Hz, 2H), 6.31 (d, J = 7.8 Hz, 1H), 4.54-4.46 (m, 1H), 3.87-3.81 (m, 1H), 3.66-3.61 (m, 1H), 3.48 (s, 3H), 3.29 (s, 3H), 3.10-3.02 (m, 4H), 2.48-2.40 (m, 5H), 2.22 (s, 3H), 1.57-1.43 (m, 1H), 1.26 (s, 3H), 1.22 (s, 3H), 1.01 (s, 3H). | LCMS (ES, m/z): 577 [M + H]+, Rt 0.567 min. | |
| 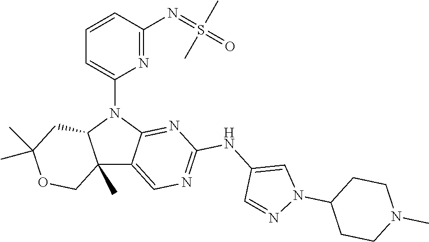 | 101, 102 | 1H NMR (400 MHz, DMSO-d6) δ 9.11 (s, 1H), 7.97-7.91 (m, 3H), 7.55-7.45 (m, 2H), 6.32 (d, J = 7.6 Hz, 1H), 4.52-4.48 (m, 1H), 4.06- 3.98 (m, 1H), 3.80-3.77 (m, 1H), 3.63-3.60 (m, 1H), 3.50 (s, 3H), 3.29 (s, 3H), 2.85-2.82 (m, 2H), 2.43-2.39 (m, 1H), 2.32 (s, 3H), 2.05-1.82 (m, 6H), 1.51-1.45 (m, 1H), 1.23 (s, 3H), 1.20 (s, 3H), 0.99 (s, 3H). | LCMS (ES, m/z): 566 [M + H]+. Rt 0.555 min. | Column: CHIRAL ART Cellulose-SC, 2*25 cm, 5 um; Mobile Phase A: Hex(0.5% 2 M NH3—MeOH)-- HPLC, Mobile Phase B: EtOH-- HPLC; Flow rate: 20 mL/min; Gradient: 50 B to 50 B in 17 min; 220/254 nm; RT1: 11.213; RT2: 14.1 |
| 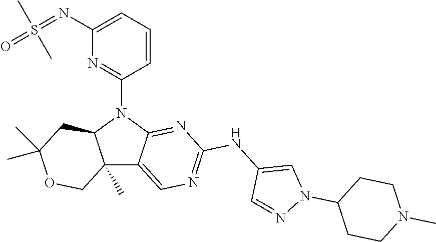 [SMILES: CN1CCC(n2cc(Nc3ncc4c(n3)N(c3cccc(N=S(C)(C)=O)n3)[C@@H]3CC(C)(C)OC[C@]43C)cn2)CC1] | | 1H NMR (400 MHz, DMSO-d6) δ 9.11 (s, 1H), 7.97-7.91 (m, 3H), 7.55-7.45 (m, 2H), 6.32 (d, J = 8.0 Hz, 1H), 4.50-4.47 (m, 1H), 4.05- 3.98 (m, 1H), 3.80-3.77 (m, 1H), 3.63-3.60 (m, 1H), 3.47 (s, 3H), 3.29 (s, 3H), 2.84-2.81 (m, 2H), 2.43-2.40 (m, 1H), 2.20 (s, 3H), 2.03-1.89 (m, 6H), 1.51-1.45 (m, 1H), 1.23 (s, 3H), 1.20 (s, 3H), 0.99 (s, 3H). | LCMS (ES, m/z): 566 [M + H]+. Rt 0.551 min. | |
| 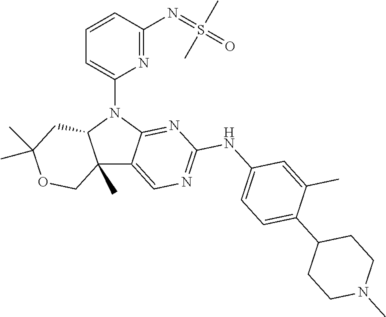 | 103, 104 | 1H NMR (300 MHz, DMSO-d6) δ 9.15 (s, 1H), 8.11 (d, J = 7.8 Hz, 1H), 8.01 (s, 1H), 7.59-7.51 (m, 2H), 7.46-7.41 (m, 1H), 7.11 (d, J = 8.4 Hz, 1H), 6.34 (d, J = 7.2 Hz, 1H), 4.57-4.52 (m, 1H), 3.86-3.82 (m, 1H), 3.67-3.63 (m, 1H), 3.49 (s, 3H), 3.30 (s, 3H), 2.95-2.92 (m, 2H), 2.61-2.54 (m, 1H), 2.47-2.42 (m, 1H), 2.29- 2.26 (m, 6H), 2.08-1.99 (m, 2H), 1.67-1.66 (m, 4H), 1.56-1.48 (m, 1H), 1.28 (s, 3H), 1.24 (s, 3H), 1.03 (s, 3H). | LCMS (ES, m/z): 590 [M + H]+. Rt 0.614 min. | Column: CHIRAL ART Cellulose- SC, 2*25 cm, 5 um; Mobile Phase A: Hex: DCM = 3:1(0.5% 2 M NH3—MeOH)-- HPLC, Mobile Phase B: EtOH-- HPLC; Flow rate: 20 mL/min; Gradient: 15 B to 15 B in 12 min; 220/254 nm; RT1: 7.053; RT2: 9.968 |
| 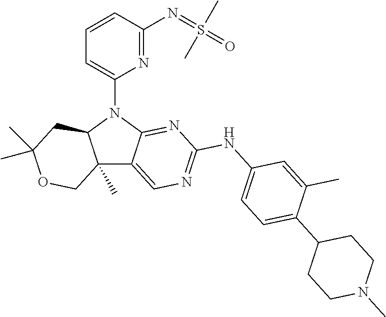 | | 1H NMR (300 MHz, DMSO-d6) δ 9.12 (s, 1H), 8.11 (d, J = 8.1 Hz, 1H), 8.01 (s, 1H), 7.63- 7.52 (m, 2H), 7.42-7.39 (m, 1H), 6.99 (d, J = 8.7 Hz, 1H), 6.35 (d, J = 7.8 Hz, 1H), 4.57- 4.52 (m, 1H), 3.87-3.82 (m, 1H), 3.66-3.61 (m, 1H), 3.50 (s, 3H), 3.31 (s, 3H), 2.87-2.83 (m, 4H), 2.59-2.50 (m, 3H), 2.48-2.40 (m, 2H), 2.33 (s, 3H), 2.25 (s, 3H), 1.56-1.48 (m, 1H), 1.28 (s, 3H), 1.24 (s, 3H), 1.03 (s, 3H). | LCMS (ES, m/z): 590 [M + H]+. Rt 0.612 min. | |
| 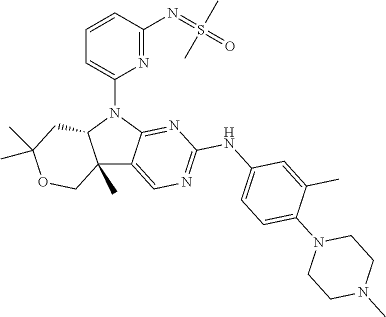 | 105, 106 | 1H NMR (300 MHz, DMSO-d6) δ 9.12 (s, 1H), 8.11 (d, J = 8.1 Hz, 1H), 8.01 (s, 1H), 7.62- 7.52 (m, 2H), 7.41 (d, J = 8.7 Hz, 1H), 6.98 (d, J = 8.7 Hz, 1H), 6.35 (d, J = 7.5 Hz, 1H), 4.57- 4.52 (m, 1H), 3.87-3.82 (m, 1H), 3.66-3.61 (m, 1H), 3.50 (s, 3H), 3.31 (s, 3H), 2.83-2.80 (m, 4H), 2.51-2.42 (m, 5H), 2.25 (s, 6H), 1.56-1.48 (m, 1H), 1.28 (s, 3H), 1.24 (s, 3H), 1.03 (s, 3H). | LCMS (ES, m/z): 591 [M + H]+. Rt 0.672 min. | Column: CHIRAL ART Cellulose- SC, 2*25 cm, 5 um; Mobile Phase A: Hex: DCM = 3:1(0.5% 2 M NH3—MeOH)-- HPLC, Mobile Phase B: EtOH--HPLC; Flow rate: 20 mL/min; Gradient: 10 B to 10 B in 16 min; 220/254 nm; RT1: 12.277; RT2: 14.446 |
| 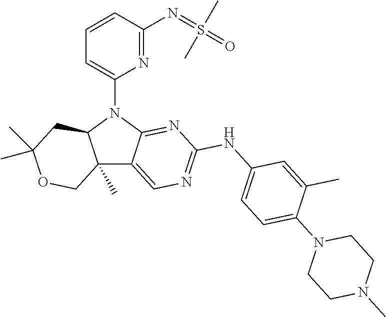 | | 1H NMR (300 MHz, DMSO-d6) δ 9.12 (s, 1H), 8.11 (d, J = 8.1 Hz, 1H), 8.01 (s, 1H), 7.63- 7.52 (m, 2H), 7.42-7.39 (m, 1H), 6.99 (d, J = 8.7 Hz, 1H), 6.35 (d, J = 7.8 Hz, 1H), 4.57- 4.52 (m, 1H), 3.87-3.82 (m, 1H), 3.66-3.61 (m, 1H), 3.50 (s, 3H), 3.31 (s, 3H), 2.87-2.83 (m, 4H), 2.59-2.50 (m, 3H), 2.48-2.40 (m, 2H), 2.33 (s, 3H), 2.25 (s, 3H), 1.56-1.48 (m, 1H), 1.28 (s, 3H), 1.24 (s, 3H), 1.03 (s, 3H). | LCMS (ES, m/z): 591 [M + H]+. Rt 0.679 min. | |
| 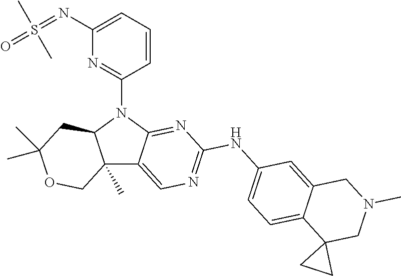 [SMILES: CN1Cc2cc(Nc3ncc4c(n3)N(c3cccc(N=S(C)(C)=O)n3)[C@@H]3CC(C)(C)OC[C@]43C)ccc2C2(CC2)C1] | 107, 108 | 1H NMR (400 MHz, DMSO-d6) δ 9.18 (s, 1H), 8.07 (d, J = 8.0 Hz, 1H), 8.00 (s, 1H), 7.55-7.51 (m, 2H), 7.35 (d, J = 8.4 Hz, 1H), 6.62 (d, J = 8.8 Hz, 1H), 6.34 (d, J = 7.6 Hz, 1H), 4.55-4.52 (m, 1H), 3.84-3.81 (m, 1H), 3.66-3.63 (m, 1H), 3.54-3.51 (m, 2H), 3.30 (s, 3H), 2.50 (s, 3H), 2.43 (s, 3H), 2.31-2.30 (m, 3H), 1.54-1.47 (m, 1H), 1.27 (s, 3H), 1.23 (s, 3H), 1.02 (s, 3H), 0.90-0.87 (m, 2H), 0.82-0.79 (m, 2H). | LCMS (ES, m/z): 574 [M + H]+. Rt 0.602 min. | Column: CHIRALPAK IE, 2*25 cm, 5 um; Mobile Phase A: HEX: DCM = 3:1(0.3% IPA)-- HPLC, Mobile Phase B: EtOH--HPLC; Flow rate: 20 mL/min; Gradient: 70 B to 70 B in 17 min; 220/254 nm; RT1: 10.167; RT2: 14.928 |
| 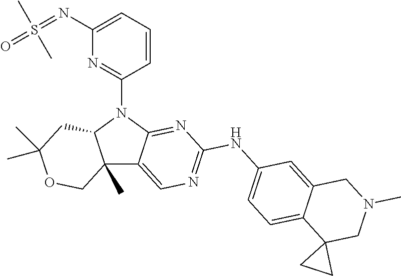 [SMILES: CN1Cc2cc(Nc3ncc4c(n3)N(c3cccc(N=S(C)(C)=O)n3)[C@H]3CC(C)(C)OC[C@@]43C)ccc2C2(CC2)C1] | | 1H NMR (300 MHz, DMSO-d6) δ 9.19 (s, 1H), 8.08 (d, J = 8.1 Hz, 1H), 8.01 (s, 1H), 7.56-7.51 (m, 2H), 7.37 (d, J = 8.4 Hz, 1H), 6.63 (d, J = 8.4 Hz, 1H), 6.35 (d, J = 7.5 Hz, 1H), 4.57-4.52 (m, 1H), 3.85-3.81 (m, 1H), 3.67-3.63 (m, 1H), 3.55-3.30 (m, 5H), 2.50 (s, 3H), 2.44 (s, 3H), 2.33-2.32 (m, 3H), 1.56-1.48 (m, 1H), 1.27 (s, 3H), 1.23 (s, 3H), 1.03 (s, 3H), 0.91-0.88 (m, 2H), 0.83-0.80 (m, 2H). | LCMS (ES, m/z): 574 [M + H]+. Rt 0.598 min. | |
| 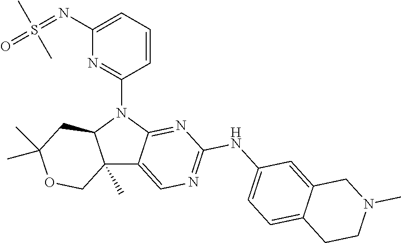 [SMILES: CN1CCc2ccc(Nc3ncc4c(n3)N(c3cccc(N=S(C)(C)=O)n3)[C@@H]3CC(C)(C)OC[C@]43C)cc2C1] | 109, 110 | 1H NMR (300 MHz, DMSO-d6) δ 9.20 (s, 1H), 8.08-8.02 (m, 2H), 7.54 (t, J = 7.8 Hz, 2H), 7.40 (d, J = 8.4 Hz, 1H), 7.01 (d, J = 8.1 Hz, 1H), 6.35 (d, J = 7.8 Hz, 1H), 4.57-4.52 (m, 1H), 3.86-3.81 (m, 1H), 3.67-3.62 (m, 1H), 3.50-3.46 (m, 5H), 3.31 (s, 3H), 2.78-2.74 (m, 2H), 2.61-2.57 (m, 2H), 2.47-2.42 (m, 1H), 2.35 (s, 3H), 1.56-1.48 (m, 1H), 1.28 (s, 3H), 1.24 (s, 3H), 1.03 (s, 3H). | LCMS (ES, m/z): 548 [M + H]+. Rt 0.576 min. | Column: CHIRALPAK IG, 2*25 cm, 5 um; Mobile Phase A: HEX: DCM = 3:1(0.3% IPA)--HPLC, Mobile Phase B: EtOH--HPLC; Flow rate: 20 mL/min; Gradient: 50 B to 50 B in 16 min; 220/254 nm; RT1: 9.022; RT2: 14.538 |
| 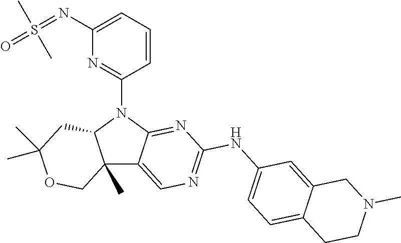 [SMILES: CN1CCc2ccc(Nc3ncc4c(n3)N(c3cccc(N=S(C)(C)=O)n3)[C@H]3CC(C)(C)OC[C@@]43C)cc2C1] | | 1H NMR (300 MHz, DMSO-d6) δ 9.20 (s, 1H), 8.08-8.02 (m, 2H), 7.54 (t, J = 7.8 Hz, 2H), 7.40 (d, J = 9.9 Hz, 1H), 7.01 (d, J = 8.4 Hz, 1H), 6.35 (d, J = 7.5 Hz, 1H), 4.57-4.52 (m, 1H), 3.86-3.82 (m, 1H), 3.67-3.61 (m, 1H), 3.50-3.46 (m, 5H), 3.31 (s, 3H), 2.78-2.74 (m, 2H), 2.61-2.57 (m, 2H), 2.47-2.42 (m, 1H), 2.35 (s, 3H), 1.56-1.48 (m, 1H), 1.28 (s, 3H), 1.24 (s, 3H), 1.03 (s, 3H). | LCMS (ES, m/z): 548 [M + H]+. Rt 0.580 min. | |
| 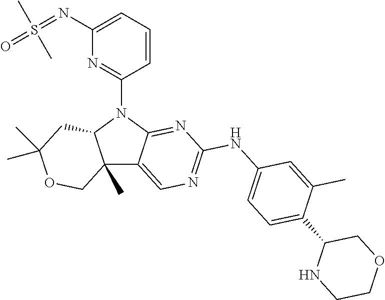 [SMILES: Cc1cc(Nc2ncc3c(n2)N(c2cccc(N=S(C)(C)=O)n2)[C@H]2CC(C)(C)OC[C@@]32C)ccc1[C@@H]1COCCN1] | 113, 114 | 1H NMR (300 MHz, DMSO-d6) δ 9.22 (s, 1H), 8.09 (d, J = 8.4 Hz, 1H), 8.02 (s, 1H), 7.59- 7.52 (m, 2H), 7.43-7.39 (m, 2H), 6.33 (d, J = 7.8 Hz, 1H), 4.56-4.52 (m, 1H), 3.92-3.81 (m, 2H), 3.76-3.63 (m, 3H), 3.49-3.46 (m, 4H), 3.30 (s, 3H), 3.11 (t, J = 10.5 Hz, 1H), 2.92- 2.89 (m, 2H), 2.50-2.44 (m, 2H), 2.32 (s, 3H), 1.53-1.48 (m, 1H), 1.27 (s, 3H), 1.23 (s, 3H), 1.02 (s, 3H). | LCMS (ES, m/z): 578 [M + H]+, Rt 0.579 min. | Column: CHIRAL ART Cellulose- SC, 2*25 cm, 5 um; Mobile Phase A: Hex: DCM = 3:1 (0.5% 2 M NH3—MeOH)-- HPLC, Mobile Phase B: EtOH-- HPLC; Flow rate: 20 mL/min; Gradient: 20% B to 0% B in 0 min; Wave Length: 220/254 nm; RT1(min): 5.49; RT2(min): 6.7; |
| 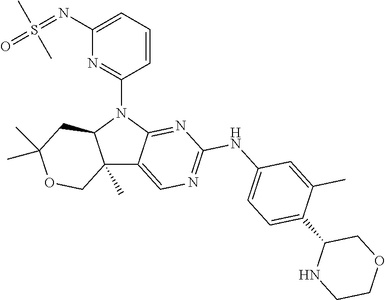 [SMILES: Cc1cc(Nc2ncc3c(n2)N(c2cccc(N=S(C)(C)=O)n2)[C@@H]2CC(C)(C)OC[C@]32C)ccc1[C@@H]1COCCN1] | | 1H NMR (300 MHz, DMSO-d6) δ 9.21 (s, 1H), 8.09 (d, J = 8.1 Hz, 1H), 8.02 (s, 1H), 7.59- 7.52 (m, 2H), 7.43-7.39 (m, 2H), 6.34 (d, J = 7.8 Hz, 1H), 4.56-4.52 (m, 1H), 3.92-3.81 (m, 2H), 3.77-3.63 (m, 3H), 3.49-3.46 (m, 4H), 3.30 (s, 3H), 3.11 (t, J = 10.2 Hz, 1H), 2.93- 2.89 (m, 2H), 2.50-2.42 (m, 2H), 2.32 (s, 3H), 1.53-1.48 (m, 1H), 1.27 (s, 3H), 1.24 (s, 3H), 1.02 (s, 3H). | LCMS (ES, m/z): 578 [M + H]+, Rt 0.592 min. | |
| 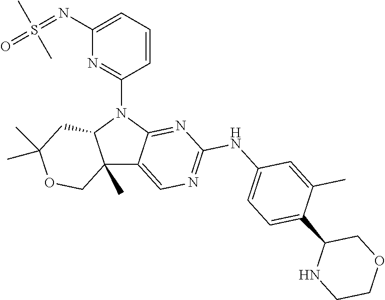 [SMILES: Cc1cc(Nc2ncc3c(n2)N(c2cccc(N=S(C)(C)=O)n2)[C@H]2CC(C)(C)OC[C@@]32C)ccc1[C@H]1COCCN1] | 115, 120 | 1H NMR (300 MHz, DMSO-d6) δ 9.22 (s, 1H), 8.10-8.02 (m, 2H), 7.59-7.52 (m, 2H), 7.47- 7.39 (m, 2H), 6.34 (d, J = 7.8 Hz, 1H), 4.57- 4.52 (m, 1H), 3.92-3.81 (m, 2H), 3.77-3.63 (m, 3H), 3.49-3.43 (m, 4H), 3.30 (s, 3H), 3.11 (t, J = 10.2 Hz, 1H), 2.96-2.89 (m, 2H), 2.56- 2.51 (m, 1H), 2.47-2.42 (m, 1H), 2.32 (s, 3H), 1.56-1.50 (m, 1H), 1.28 (s, 3H), 1.24 (s, 3H), 1.02 (s, 3H). | LCMS (ES, m/z): 578 [M + H]+, Rt 0.589 min. | Column: CHIRAL ART Cellulose- SB, 2*25 cm, 5 um; Mobile Phase A: MtBE(0.5% 2 M NH3—MeOH)-- HPLC, Mobile Phase B: EtOH-- HPLC; Flow rate: 20 mL/min; Gradient: 5% B to 5% B in 15 min; Wave Length: 220/254 nm; RT1(min): 10.32; RT2(min): 12.66; |
| 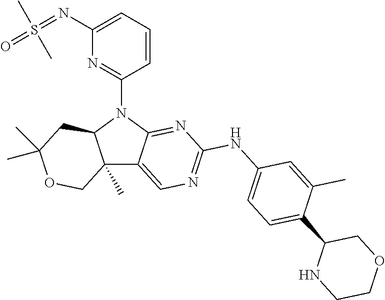 [SMILES: Cc1cc(Nc2ncc3c(n2)N(c2cccc(N=S(C)(C)=O)n2)[C@@H]2CC(C)(C)OC[C@]32C)ccc1[C@H]1COCCN1] | | 1H NMR (300 MHz, DMSO-d6) δ 9.21 (s, 1H), 8.08 (d, J = 6.0 Hz, 1H), 8.02 (s, 1H), 7.59- 7.52 (m, 2H), 7.45-7.39 (m, 2H), 6.33 (d, J = 5.6 Hz, 1H), 4.56-4.52 (m, 1H), 3.92-3.90 (m, 1H), 3.89-3.81 (m, 1H), 3.76-3.73 (m, 1H), 3.68-3.63 (m, 2H), 3.49-3.46 (m, 4H), 3.30 (s, 3H), 3.11 (t, J = 7.8 Hz, 1H), 2.92-2.89 (m, 2H), 2.50-2.44 (m, 2H), 2.32 (s, 3H), 1.56- 1.50 (m, 1H), 1.27 (s, 3H), 1.23 (s, 3H), 1.02 (s, 3H). | LCMS (ES, m/z): 578 [M + H]+, Rt 0.584 min. | |
| 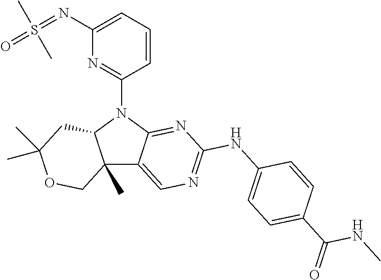 [SMILES: CNC(=O)c1ccc(Nc2ncc3c(n2)N(c2cccc(N=S(C)(C)=O)n2)[C@H]2CC(C)(C)OC[C@@]32C)cc1] | 116, 117 | 1H NMR (300 MHz, DMSO-d6) δ 9.60 (s, 1H), 8.22 (br, 1H), 8.11-8.08 (m, 2H), 7.86-7.79 (m, 4H), 7.68-7.63 (m, 1H), 6.36 (d, J = 7.8 Hz, 1H), 4.59-4.52 (m, 1H), 3.88-3.83 (m, 1H), 3.69-3.64 (m, 1H), 3.50 (s, 3H), 3.32 (s, 3H), 2.79 (d, J = 4.8 Hz, 3H), 2.50-2.42 (m, 1H), 1.58-1.52 (m, 1H), 1.30 (s, 3H), 1.24 (s, 3H), 1.03 (s, 3H). | LCMS (ES, m/z): 536 [M + H]+, Rt 0.691 min. | Column: CHIRAL ART Cellulose- SC, 2 × 25 cm, 5 um; Mobile Phase A: Hex: DCM = 3:1(0.5% 2 M NH3—MeOH)-- HPLC, Mobile Phase B: EtOH-- HPLC(50% to 50% in 8.7 min); Flow rate: 20 mL/min; Wave Length: 220/254 nm; RT1(min): 5.54; RT2(min): 7.44 |
| 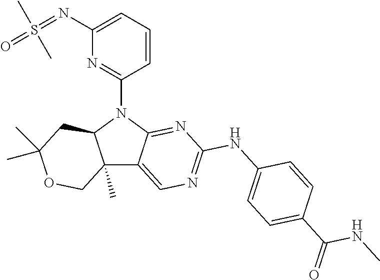 [SMILES: CNC(=O)c1ccc(Nc2ncc3c(n2)N(c2cccc(N=S(C)(C)=O)n2)[C@@H]2CC(C)(C)OC[C@]32C)cc1] | | 1H NMR (300 MHz, DMSO-d6) δ 9.60 (s, 1H), 8.22 (br, 1H), 8.11-8.08 (m, 2H), 7.86-7.75 (m, 4H), 7.63-7.58 (m, 1H), 6.36 (d, J = 7.8 Hz, 1H), 4.59-4.55 (m, 1H), 3.86-3.81 (m, 1H), 3.67-3.62 (m, 1H), 3.50 (s, 3H), 3.31 (s, 3H), 2.78 (d, J = 4.5 Hz, 3H), 2.50-2.44 (m, 1H), 1.59-1.52 (m, 1H), 1.30 (s, 3H), 1.24 (s, 3H), 1.03 (s, 3H). | LCMS (ES, m/z): 536 [M + H]+, Rt 0.690 min. | |
| 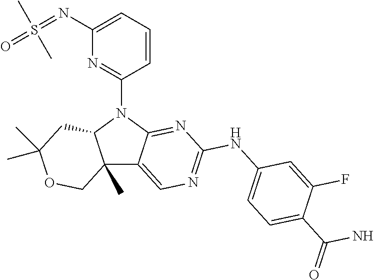 | 118, 119 | 1H NMR (300 MHz, DMSO-d6) δ 9.84 (s, 1H), 8.11 (s, 1H), 8.08- 7.82 (m, 3H), 7.79-7.61 (m, 2H), 7.53-7.40 (m, 1H), 6.36 (d, J = 7.8 Hz, 1H), 4.67-4.48 (m, 1H), 3.95-3.63 (m, 2H), 3.49 (s, 3H), 3.30 (s, 3H), 2.82-2.78 (m, 3H), 2.39 -2.37 (m, 1H), 1.67-1.48 (m, 1H), 1.29 (s, 3H), 1.23 (s, 3H), 1.04 (s, 3H). | LCMS (ES, m/z): 554 [M + H]+. Rt 0.657 min | Column: CHIRAL ART Cellulose- SC, 2*25 cm, 5 um; Mobile Phase A: Hex: DCM = 3:1(0.5% 2 M NH3—MeOH)-- HPLC, Mobile Phase B: EtOH-- HPLC; Flow rate: 20 mL/min; Gradient: 20% B to 20% B in 9 min; Wave Length: 220/254 nm; RT1(min): 6.61; RT2(min): 8.02; |
| 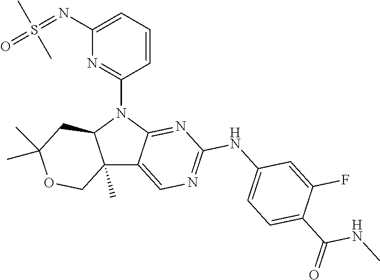 [SMILES: CNC(=O)c1ccc(Nc2ncc3c(n2)N(c2cccc(N=S(C)(C)=O)n2)[C@@H]2CC(C)(C)OC[C@]32C)cc1F] | | 1H NMR (300 MHz, DMSO-d6) δ 9.83 (s, 1H), 8.11(s, 1H), 8.04 (d, J = 8.1 Hz, 1H), 8.01-7.89 (m, 2H), 7.69-7.50 (m, 2H), 7.49-7.30 (m, 1H), 6.36 (d, J = 7.8 Hz, 1H), 4.65-4.47 (m, 1H), 3.86-3.81 (m, 1H), 3.67-3.62 (m, 1H), 3.49 (s, 3H), 3.30 (s, 3H), 2.77-2.75 (m, 3H), 2.49-2.47 (m, 1H), 1.68-1.51 (m, 1H), 1.29 (s, 3H), 1.23 (s, 3H), 1.01 (s, 3H). | LCMS (ES, m/z): 554 [M + H]+. Rt 0.652 min. | |
| 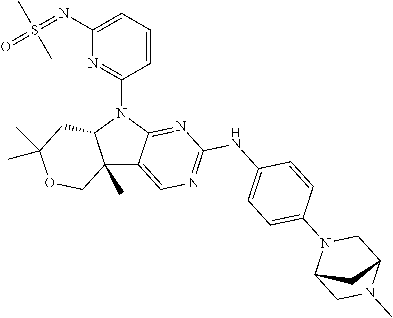 [SMILES: CN1C[C@H]2C[C@@H]1CN2c1ccc(Nc2ncc3c(n2)N(c2cccc(N=S(C)(C)=O)n2)[C@H]2CC(C)(C)OC[C@@]32C)cc1] | 121 122 | 1H NMR (300 MHz, DMSO-d6) δ 8.88 (s, 1H), 8.13 (d, J = 8.1 Hz, 1H), 7.95 (s, 1H), 7.55- 7.44 (m, 3H), 6.55 (d, J = 8.4 Hz, 2H), 6.32 (d, J = 7.5 Hz, 1H), 4.54-4.50 (m, 1H), 4.24-4.19 (m, 1H), 3.84-3.79 (m, 1H), 3.66-3.61 (m, 1H), 3.49-3.39 (m, 4H), 3.30-3.33 (sm, 4H), 3.13-3.08 (m, 1H), 2.78-2.75 (m, 1H), 2.44- 2.42 (m, 1H), 2.28-2.25 (m, 4H), 1.87-1.84 (m, 1H), 1.77-1.74 (m, 1H), 1.54-1.47 (m, 1H), 1.26 (s, 3H), 1.24 (s, 3H), 1.03 (s, 3H). | LCMS (ES, m/z): 589 [M + H]+. Rt 0.590 min. | Column: CHIRAL ART Amylose-SA, 2*25 cm, 5 um; Mobile Phase A: Hex: DCM = 3:1 (0.5% 2 M NH3—MeOH)--HPLC, Mobile Phase B: EtOH--HPLC; Flow rate: 20 mL/min; Gradient: 10% B to 10% B in 22 min; Wave Length: 220/254 nm; RT1(min): 10.04; RT2(min): 19.18; Sample Solvent: EtOH--HPLC; |
| 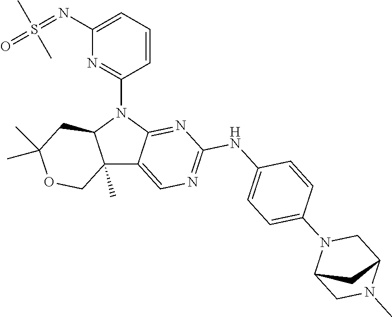 [SMILES: CN1C[C@H]2C[C@@H]1CN2c1ccc(Nc2ncc3c(n2)N(c2cccc(N=S(C)(C)=O)n2)[C@@H]2CC(C)(C)OC[C@]32C)cc1] | | 1H NMR (300 MHz, DMSO-d6) δ 8.88 (s, 1H), 8.13 (d, J = 8.1 Hz, 1H), 7.95 (s, 1H), 7.55- 7.44 (m, 3H), 6.55 (d, J = 8.7 Hz, 2H), 6.32 (d, J = 7.8 Hz, 1H), 4.54-4.50 (m, 1H), 4.24-4.18 (m, 1H), 3.87-3.82 (m, 1H), 3.66-3.61 (m, 1H), 3.49 (s, 3H), 3.40-3.33 (m, 1H), 3.33- 3.30 (m, 4H), 3.13-3.08 (m, 1H), 2.78-2.75 (m, 1H), 2.44-2.42 (m, 1H), 2.28-2.25 (m, 4H), 1.87-1.84 (m, 1H), 1.77-1.74 (m, 1H), 1.54-1.47 (m, 1H), 1.26 (s, 3H), 1.24 (s, 3H), 1.03 (s, 3H). | LCMS (ES, m/z): 589 [M + H]+. Rt 0.592 min. | |
| 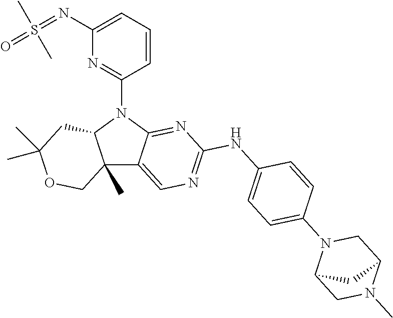 [SMILES: CN1C[C@@H]2C[C@H]1CN2c1ccc(Nc2ncc3c(n2)N(c2cccc(N=S(C)(C)=O)n2)[C@H]2CC(C)(C)OC[C@@]32C)cc1] | 123, 124 | 1H NMR (300 MHz, DMSO-d6) δ 8.89 (s, 1H), 8.11 (d, J = 8.1 Hz, 1H), 7.94 (s, 1H), 7.56- 7.41 (m, 3H), 6.58-6.48 (m, 2H), 6.30 (d, J = 7.8 Hz, 1H), 4.56-4.45 (m, 1H), 4.23-4.19 (m, 1H), 3.85-3.81 (m, 1H), 3.67-3.62 (m, 1H), 3.49 (s, 3H), 3.40-3.34 (m, 7H), 3.15-3.09 (m, 1H), 2.78-2.74 (m, 1H), 2.25 (s, 3H), 1.89- 1.73 (m, 2H), 1.55-1.41 (m, 1H), 1.29 (s, 3H), 1.25 (s, 3H), 1.02 (s, 3H). | LCMS (ES, m/z): 589 [M + H]+; RT: 0.596 min. | Column: CHIRAL ART Cellulose- SC, 2*25 cm, 5 um; Mobile Phase A: Hex: DCM = 3:1 (0.5% 2 M NH3—MeOH)-- HPLC, Mobile Phase B: EtOH-- HPLC; Flow rate: 20 mL/min; Gradient: 30% B to 30% B in 10.5 min; Wave Length: 220/254 nm; RT1(min): 7.09; RT2(min): 8.74; Sample Solvent: EtOH-- HPLC; Injection Volume: 0.8 mL; Number Of Runs: 4 |
| 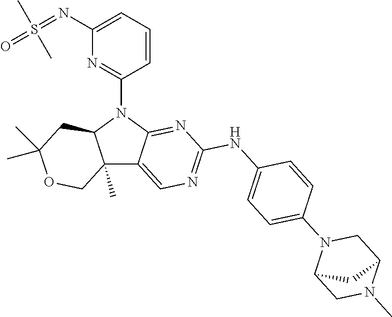 [SMILES: CN1C[C@@H]2C[C@H]1CN2c1ccc(Nc2ncc3c(n2)N(c2cccc(N=S(C)(C)=O)n2)[C@@H]2CC(C)(C)OC[C@]32C)cc1] | | 1H NMR (300 MHz, DMSO-d6) δ 8.90 (s, 1H), 8.11 (d, J = 8.1 Hz, 1H), 7.94 (s, 1H), 7.56- 7.42 (m, 3H), 6.58-6.48 (m, 2H), 6.30 (d, J = 7.8 Hz, 1H), 4.56-4.45 (m, 1H), 4.24-4.22 (m, 1H), 3.85-3.60 (m, 2H), 3.55-3.37 (m, 5H), 3.31-3.18 (m, 5H), 3.12-3.08 (m, 1H), 2.82- 2.72 (m, 1H), 2.26 (s, 3H), 1.90-1.73 (m, 2H), 1.49-1.41 (m, 1H), 1.29 (s, 3H), 1.24 (s, 3H), 1.02 (s, 3H). | LCMS (ES, m/z): 589 [M + H]+; RT: 0.599 min. | |
| 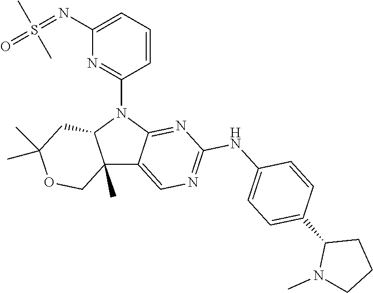 [SMILES: CN1CCC[C@H]1c1ccc(Nc2ncc3c(n2)N(c2cccc(N=S(C)(C)=O)n2)[C@H]2CC(C)(C)OC[C@@]32C)cc1] | 125, 126 | 1H NMR (300 MHz, DMSO-d6) δ 9.25 (s, 1H), 8.11 (d, J = 8.1 Hz, 1H), 8.02 (s, 1H), 7.67 (d, J = 8.1 Hz, 2H), 7.58-7.50 (m, 1H), 7.22 (d, J = 8.1 Hz, 2H), 6.34 (d, J = 8.4 Hz, 1H), 4.60- 4.50 (m, 1H), 3.90-3.60 (m, 2H), 3.50 (s, 3H), 3.31 (s, 3H), 3.20-3.10 (m, 1H), 3.02-2.92 (m, 1H), 2.49-2.40 (m, 1H), 2.25-2.15 (m, 1H), 2.12-2.05 (m, 4H), 1.90-1.70 (m, 2H), 1.65- 1.45 (m, 2H), 1.31(s, 3H), 1.25(s, 3H), 1.03 (s, 3H). | LCMS (ES, m/z): 562 [M + H]+, Rt 0.583 min. | Column: CHIRAL ART Cellulose- SC, 2*25 cm, 5 um; Mobile Phase A: Hex: DCM = 3:1(0.5% 2 M NH3—MeOH)-- HPLC, Mobile Phase B: EtOH-- HPLC; Flow rate: 20 mL/min; Gradient: 30% B to 30% B in 7 min; Wave Length: 220/254 nm; RT1(min): 5.13; RT2(min): 6.12; |
| 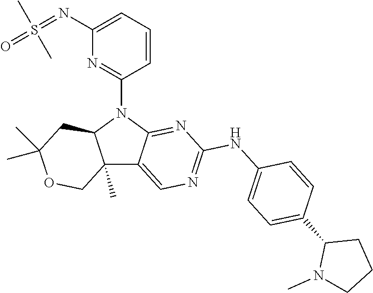 [SMILES: CN1CCC[C@H]1c1ccc(Nc2ncc3c(n2)N(c2cccc(N=S(C)(C)=O)n2)[C@@H]2CC(C)(C)OC[C@]32C)cc1] | | 1H NMR (300 MHz, DMSO-d6) δ 9.25 (s, 1H), 8.11 (d, J = 8.1 Hz, 1H), 8.02 (s, 1H), 7.67 (d, J = 8.1 Hz, 2H), 7.58-7.48 (m, 1H), 7.22 (d, J = 8.1 Hz, 2H), 6.34 (d, J = 8.1 Hz, 1H), 4.60- 4.50 (m, 1H), 3.88-3.60 (m, 2H), 3.50 (s, 3H), 3.31 (s, 3H), 3.20-3.10 (m, 1H), 3.05-2.90 (m, 1H), 2.50-2.40 (m, 1H), 2.25-2.14 (m, 1H), 2.12-2.05 (m, 4H), 1.90-1.70 (m, 2H), 1.60- 1.45 (m, 2H), 1.30 (s, 3H), 1.24 (s, 3H), 1.03 (s, 3H). | LCMS (ES, m/z): 562 [M + H]+, Rt 0.592 min. | |
| 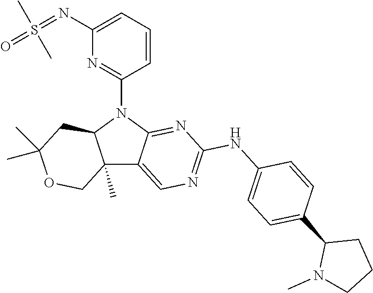 [SMILES: CN1CCC[C@@H]1c1ccc(Nc2ncc3c(n2)N(c2cccc(N=S(C)(C)=O)n2)[C@@H]2CC(C)(C)OC[C@]32C)cc1] | 127, 128 | 1H NMR (300 MHz, DMSO-d6) δ 9.35 (s, 1H), 8.12-8.04 (m, 2H), 7.78-7.71 (m, 2H), 7.56 (t, J = 8.1 Hz, 1H), 7.34-7.38 (m, 2H), 6.35 (d, J = 8.1 Hz, 1H), 4.58-4.53 (m, 1H), 3.87-3.82 (m, 1H), 3.66-3.61 (m, 1H), 3.50 (s, 3H), 3.33 (s, 3H), 2.52 (s, 3H), 2.50-2.45 (m, 1H), 2.35- 2.28 (m, 4H), 1.92-1.75 (m, 3H), 1.57-1.50 (m, 1H), 1.29 (s, 3H), 1.24 (s, 3H) 1.03 (s, 3H). | LCMS (ES, m/z): 562 [M + H]+, Rt 0.587 min. | Column: CHIRALPAK ID, 2*25 cm, 5 um; Mobile Phase A: Hex(0.5% 2 M NH3—MeOH)-- HPLC, Mobile Phase B: EtOH-- HPLC; Flow rate: 20 mL/min; Gradient: 30% B to 30% B in 22 min; Wave Length: 220/254 nm; RT1(min): 12.40; RT2(min): 14.49; |
| 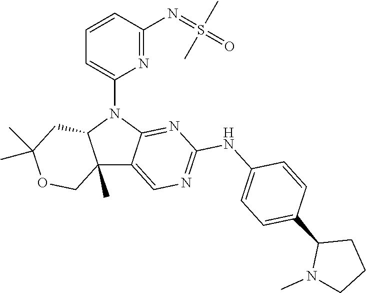 [SMILES: CN1CCC[C@@H]1c1ccc(Nc2ncc3c(n2)N(c2cccc(N=S(C)(C)=O)n2)[C@H]2CC(C)(C)OC[C@@]32C)cc1] | | 1H NMR (300 MHz, DMSO-d6) δ 9.33 (s, 1H), 8.12-8.04 (m, 2H), 7.77-7.72 (m, 2H), 7.55 (t, J = 7.8 Hz, 1H), 7.29-7.22 (m, 2H), 6.34 (d, J = 7.8 Hz, 1H), 4.57-4.51 (m, 1H), 3.87-3.82 (m, 1H), 3.66-3.61 (m, 1H), 3.50 (s, 3H), 3.31 (s, 3H), 3.18-3.12 (m, 1H), 2.51 (s, 3H), 2.50- 2.45 (m, 1H), 2.28-2.17 (m, 3H), 1.98-1.68 (m, 3H), 1.57-1.49 (m, 1H), 1.28 (s, 3H), 1.24 (s, 3H), 1.03 (s, 3H). | LCMS (ES, m/z): 562 [M + H]+, Rt 0.590 min. | |
| 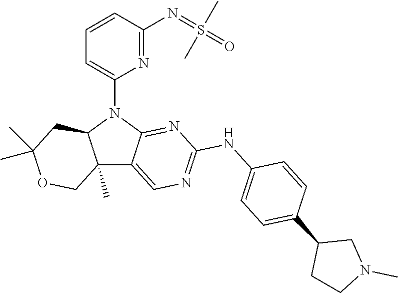 | 129, 130 | 1H NMR (300 MHz, DMSO-d6) δ 9.21 (s, 1H), 8.11 (d, J = 7.8 Hz, 1H), 8.01 (s, 1H), 7.64 (d, J = 7.8 Hz, 2H), 7.55 (t, J = 7.8 Hz, 1H), 7.17 (d, J = 7.8 Hz, 2H), 6.33 (d, J = 7.5 Hz, 1H), 4.55-4.48 (m, 1H), 3.87-3.82 (m, 1H), 3.66-3.61 (m, 1H), 3.49 (s, 2H), 3.33-3.21 (m, 5H), 2.86 (t, J = 8.4 Hz, 1H), 2.69-2.51 (m, 2H), 2.44- 2.30 (m, 2H), 2.23 (s, 4H), 1.79-1.70 (m, 1H), 1.59-1.45 (m, 1H), 1.27 (s, 3H), 1.24 (s, 3H), 1.03 (s, 3H). | LCMS (ES, m/z): 562 [M + H]+, Rt 0.583 min. | Column: CHIRAL ART Cellulose- SB, 2*25 cm, 5 um; Mobile Phase A: Hex(0.5% 2 M NH3—MeOH)-- HPLC, Mobile Phase B: EtOH-- HPLC; Flow rate: 20 mL/min; Gradient: 25% B to 25% B in 25 min; Wave Length: 220/254 nm; RT1(min): 15.40; RT2(min): 19.79; |
| 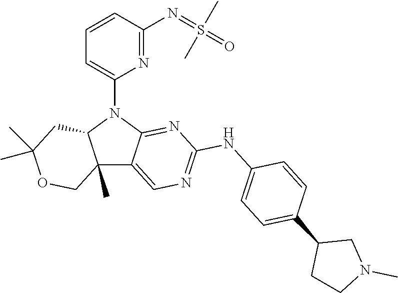 | | 1H NMR (300 MHz, DMSO-d6) δ 9.21 (s, 1H), 8.12 (d, J = 7.8 Hz, 1H), 8.01 (s, 1H), 7.64 (d, J = 7.8 Hz, 2H), 7.55 (t, J = 7.8 Hz, 1H), 7.18 (d, J = 7.8 Hz, 2H), 6.33 (d, J = 7.8 Hz, 1H), 4.54-4.52 (m, 1H), 3.87-3.82 (m, 1H), 3.66-3.61 (m, 1H), 3.49 (s, 2H), 3.30-3.24 (m, 5H), 2.86 (t, J = 7.8 Hz, 1H), 2.69-2.51 (m, 2H), 2.44- 2.34 (m, 2H), 3.30-2.17 (m, 4H), 1.79-1.70 (m, 1H), 1.59-1.45 (m, 1H), 1.28 (s, 3H), 1.24 (s, 3H), 1.03 (s, 3H). | LCMS (ES, m/z): 562 [M + H]+, Rt 0.580 min. | |
| 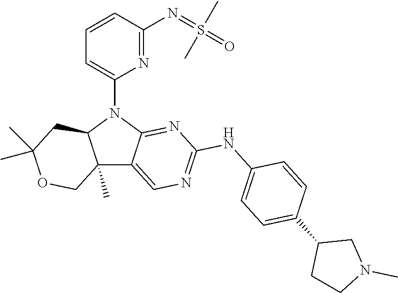 | 133, 134 | 1H NMR (300 MHz, DMSO-d6) δ 9.23 (s, 1H), 8.11 (d, J = 8.1 Hz, 1H), 8.01 (s, 1H), 7.68-7.60 (m, 2H), 7.59-7.49 (m, 1H), 7.16 (d, J = 7.8 Hz, 2H), 6.33 (d, J = 7.8 Hz, 1H), 4.58-4.48 (m, 1H), 3.87-3.61 (m, 2H), 3.49 (s, 3H), 3.33-3.30 (m, 5H), 2.92-2.80 (m, 1H), 2.69- 2.50 (m, 2H), 2.42-2.15 (m, 5H), 1.84-1.66 (m, 1H), 1.58-1.44 (m, 1H), 1.27 (s, 3H), 1.23 (s, 3H), 1.02 (s, 3H). | LCMS (ES, m/z): 562 [M + H]+; RT: 0.583 min. | Column: CHIRAL ART Cellulose- SB, 2*25 cm, 5 um; Mobile Phase A: Hex(0.5% 2 M NH3—MeOH)-- HPLC, Mobile Phase B: EtOH-- HPLC; Flow rate: 20 mL/min; Gradient: 25% B to 25% B in 26 min; Wave Length: 220/254 nm; RT1(min): 15.82; RT2(min): 20.65; |
| 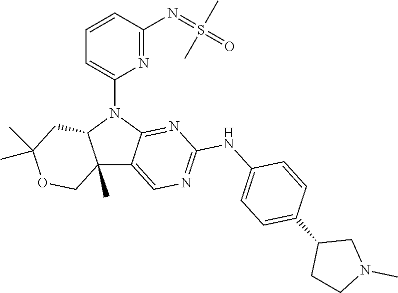 | | 1H NMR (300 MHz, DMSO-d6) δ 9.23 (s, 1H), 8.11 (d, J = 8.1 Hz, 1H), 8.01 (s, 1H), 7.69- 7.60 (m, 2H), 7.59-7.49 (m, 1H), 7.16 (d, J = 7.8 Hz, 2H), 6.33 (d, J = 7.8 Hz, 1H), 4.59- 4.48 (m, 1H), 3.87-3.61 (m, 2H), 3.49 (s, 3H), 3.32-3.19 (m, 5H), 2.92-2.57 (m, 3H), 2.46- 2.15 (m, 5H), 1.84-1.67 (m, 1H), 1.58-1.44 (m, 1H), 1.27 (s, 3H), 1.23 (s, 3H), 1.02 (s, 3H). | LCMS (ES, m/z): 562 [M + H]+; RT: 0.581 min. | |
| 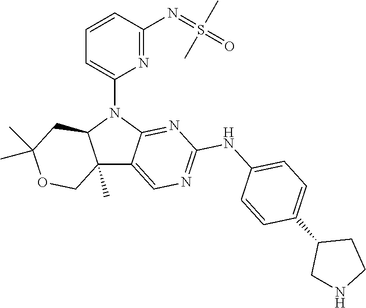 [SMILES: CC1(C)C[C@H]2N(c3cccc(N=S(C)(C)=O)n3)c3nc(Nc4ccc([C@@H]5CCNC5)cc4)ncc3[C@@]2(C)CO1] | 131, 132 | 1H NMR (300 MHz, DMSO-d6) δ 9.30-9.20 (m, 1H), 8.12 (d, J = 8.1 Hz, 1H), 8.02 (s, 1H), 7.73-7.61 (m, 2H), 7.60-7.50 (m, 1H), 7.21- 7.13 (m, 2H), 6.33 (d, J = 7.8 Hz, 1H), 4.58- 4.49 (m, 1H), 3.89-3.79 (m, 1H), 3.70-3.57 (m, 2H), 3.50 (s, 3H), 3.33-3.28 (m, 4H), 3.24-3.10 (m, 1H), 3.10-2.85 (m, 2H), 2.65- 2.56 (m, 1H), 2.48-2.40 (m, 1H), 2.20-2.05 (m, 1H), 1.75-1.62 (m, 1H), 1.59-1.45 (m, 1H), 1.28 (s, 3H), 1.24 (s, 3H), 1.03 (s, 3H). | LCMS (ES, m/z): 548 [M + H]+, Rt 0.583 min. | Column: CHIRAL ART Cellulose- SB, 2 × 25 cm, 5 um; Mobile Phase A: HEX (0.5% 2 M NH3—MeOH) --HPLC, Mobile Phase B: EtOH--HPLC; Flow rate: 20 mL/min; Gradient: 25% B to 25% B in 24 min; Wave Length: 220/254 nm; RT1(min): 15.22; RT2(min): 19.37; |
| 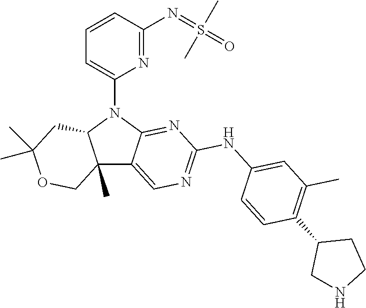 | | 1H NMR (300 MHz, DMSO-d6) δ 9.30-9.20 (m, 1H), 8.12 (d, J = 8.4 Hz, 1H), 8.02 (s, 1H), 7.70-7.61 (m, 2H), 7.60-7.50 (m, 1H), 7.21- 7.13 (m, 2H), 6.33 (d, J = 8.4 Hz, 1H), 4.58- 4.49 (m, 1H), 3.90-3.80 (m, 1H), 3.70-3.57 (m, 2H), 3.50 (s, 3H), 3.28-3.33 (m, 4H), 3.24-3.07 (m, 1H), 3.07-2.86 (m, 2H), 2.68- 2.56 (m, 1H), 2.48-2.40 (m, 1H), 2.25-2.04 (m, 1H), 1.76-1.62 (m, 1H), 1.59-1.46 (m, 1H), 1.28 (s, 3H), 1.24 (s, 3H), 1.03 (s, 3H). | LCMS (ES, m/z): 548 [M + H]+, Rt 0.583 min. | |
| 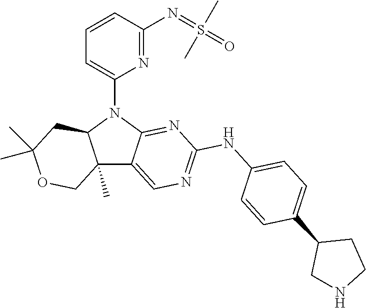 [SMILES: CC1(C)C[C@H]2N(c3cccc(N=S(C)(C)=O)n3)c3nc(Nc4ccc([C@H]5CCNC5)cc4)ncc3[C@@]2(C)CO1] | 137, 138 | 1H NMR (300 MHz, DMSO-d6) δ 9.27-9.22 (m, 1H), 8.11 (d, J = 8.1 Hz, 1H), 8.01 (s, 1H), 7.70-7.64 (m, 2H), 7.56 (t, J = 7.8 Hz, 1H), 7.21-7.16 (m, 2H), 6.33 (d, J = 7.8 Hz, 1H), 4.56-4.52 (m, 1H), 3.87-3.82 (m, 1H), 3.73- 3.56 (m, 2H), 3.49 (s, 3H), 3.35-3.33 (m, 4H), 3.24-2.91 (m, 3H), 2.74-2.62 (m, 1H), 2.51- 2.44 (m, 1H), 2.18-2.12 (m, 1H), 1.73-1.67 (m, 1H), 1.56-1.48 (m, 1H), 1.28 (s, 3H), 1.24 (s, 3H), 1.02 (s, 3H). | LCMS (ES, m/z): 548 [M + H]+, Rt 0.576 min. | Column: CHIRAL ART Cellulose- SB, 5*25 cm, 10 um; Mobile Phase A: Hex(0.5% 2 M NH3—MeOH)-- HPLC, Mobile Phase B: EtOH-- HPLC; Flow rate: 20 mL/min; Gradient: 25% B to 25% B in 16 min; Wave Length: 220/254 nm; RT1(min): 10.842; RT2(min): 13.781; |
| 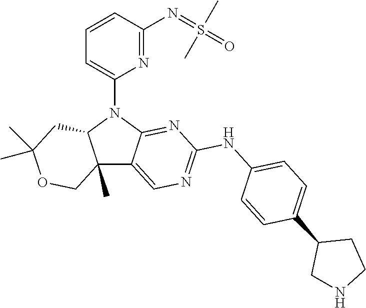 [SMILES: CC1(C)C[C@@H]2N(c3cccc(N=S(C)(C)=O)n3)c3nc(Nc4ccc([C@H]5CCNC5)cc4)ncc3[C@]2(C)CO1] | | 1H NMR (300 MHz, DMSO-d6) δ 9.27-9.24 (m, 1H), 8.11 (d, J = 8.1 Hz, 1H), 8.02 (s, 1H), 7.66 (d, J = 8.1 Hz, 2H), 7.56 (t, J = 8.1 Hz, 1H), 7.18 (d, J = 7.5 Hz, 2H), 6.33 (d, J = 7.8 Hz, 1H), 4.56-4.54 (m, 1H), 3.87-3.82 (m, 1H), 3.66-3.61 (m, 1H), 3.49-3.43 (m, 4H), 3.42 (s, 3H), 3.26-2.96 (m, 4H), 2.74-2.67 (m, 1H), 2.45-2.42 (m, 1H), 2.18-2.14 (m, 1H), 1.79-1.70 (m, 1H), 1.56-1.48 (m, 1H), 1.28 (s, 3H), 1.24 (s, 3H), 1.02 (s, 3H). | LCMS (ES, m/z): 548 [M + H]+, Rt 0.569 min. | |
| 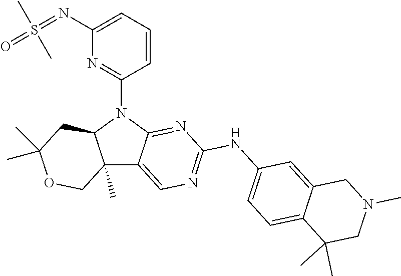 [SMILES: CN1Cc2cc(Nc3ncc4c(n3)N(c3cccc(N=S(C)(C)=O)n3)[C@@H]3CC(C)(C)OC[C@]43C)ccc2C(C)(C)C1] | 135, 136 | 1H NMR (300 MHz, DMSO-d6) δ 9.19 (s, 1H), 8.09-8.06 (m, 2H), 7.56-7.48 (m, 2H), 7.42- 7.39 (m, 1H), 7.25-7.22 (m, 1H), 6.34-6.33 (m, 1H), 4.45-4.52 (m, 1H), 3.86-3.81 (m, 1H), 3.67-3.62 (m, 1H), 3.50-3.43 (m, 5H), 3.31 (s, 3H), 2.44-2.42 (m, 1H), 2.33-2.28 (m, 5H), 1.56-1.48 (m, 1H), 1.28-1.24 (m, 12H), 1.03 (s, 3H). | LCMS (ES, m/z): 576 [M + H]+. Rt 0.603 min. | Column: CHIRALPAK IG, 2*25 cm, 5 um; Mobile Phase A: Hex: DCM = 3:1 (0.5% 2 M NH3— MeOH)--HPLC, Mobile Phase B: EtOH--HPLC; Flow rate: 16 mL/min; Gradient: 50% B to 50% B in 22 min; Wave Length: 254/220 nm; RT1(min): 13.39; RT2(min): 18.28; |
| 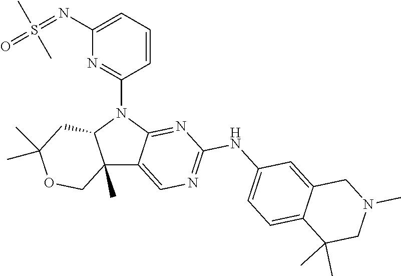 [SMILES: CN1Cc2cc(Nc3ncc4c(n3)N(c3cccc(N=S(C)(C)=O)n3)[C@H]3CC(C)(C)OC[C@@]43C)ccc2C(C)(C)C1] | | 1H NMR (300 MHz, DMSO-d6) δ 9.19 (s, 1H), 8.09-8.06 (m, 2H), 7.56-7.48 (m, 2H), 7.42- 7.39 (m, 1H), 7.25-7.22 (m, 1H), 6.36-6.33 (m, 1H), 4.56-4.52 (m, 1H), 3.86-3.81 (m, 1H), 3.67-3.61 (m, 1H), 3.50 (s, 3H), 3.43- 3.39 (m, 2H), 3.31 (s, 3H), 2.44-2.42 (m, 1H), 2.34-2.25 (m, 5H), 1.56-1.48 (m, 1H), 1.28- 1.24 (m, 13H), 1.03 (s, 3H). | LCMS (ES, m/z): 576 [M + H]+. Rt 0.612 min. | |
| 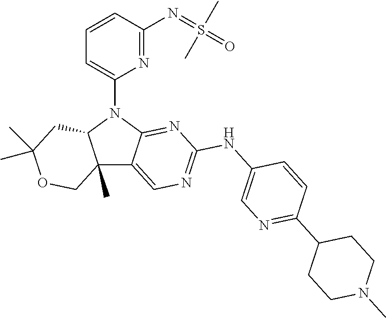 | 139, 140 | 1H NMR (300 MHz, DMSO-d6) δ 9.40 (s, 1H), 8.75 (d, J = 2.7 Hz, 1H), 8.17-8.02 (m, 3H), 7.60-7.49 (m, 1H), 7.20 (d, J = 8.7 Hz, 1H), 6.34 (d, J = 7.8 Hz, 1H), 4.60-4.49 (m, 1H), 3.89-3.62 (m, 2H), 3.49 (s, 3H), 3.40-3.33 (m, 4H), 2.89-2.81 (m, 2H), 2.46-2.19 (m, 4H), 2.08-1.94 (m, 2H), 1.86-1.65 (m, 4H), 1.59- 1.45 (m, 1H), 1.27 (s, 3H), 1.23 (s, 3H), 1.02 (s, 3H). | LCMS (ES, m/z): 577 [M + H]+; RT: 0.551 min. | Column: CHIRAL ART Cellulose- SC, 2*25 cm, 5 um; Mobile Phase A: MtBE(0.5% 2 M NH3—MeOH)-- HPLC, Mobile Phase B: EtOH-- HPLC; Flow rate: 20 mL/min; Gradient: 20% B to 20% B in 13 min; Wave Length: 254/220 nm; RT1(min): 6.56; RT2(min): 10.24; |
| 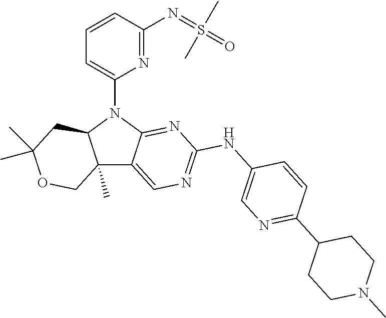 | | 1H NMR (300 MHz, DMSO-d6) δ 9.40 (s, 1H), 8.75 (d, J = 2.7 Hz, 1H), 8.16-8.02 (m, 3H), 7.60-7.49 (m, 1H), 7.20 (d, J = 8.4 Hz, 1H), 6.34 (d, J = 7.8 Hz, 1H), 4.59-4.49 (m, 1H), 3.86-3.81 (m, 1H), 3.67-3.62 (m, 1H), 3.49 (s, 3H), 3.40-3.33 (m, 4H), 2.87-2.81 (m, 2H), 2.46-2.15 (m, 4H), 2.04-1.89 (m, 2H), 1.86- 1.64 (m, 4H), 1.58-1.45 (m, 1H), 1.27 (s, 3H), 1.23 (s, 3H), 1.02 (s, 3H). | LCMS (ES, m/z): 577 [M + H]+; RT: 0.545 min. | |
| 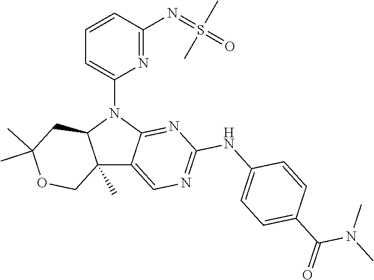 | 141, 142 | 1H NMR (300 MHz, DMSO-d6) δ 9.59 (s, 1H), 8.19-8.13 (m, 2H), 7.86 (d, J = 7.8 Hz, 2H), 7.66-7.62 (m, 1H), 7.40 (d, J = 7.8 Hz, 2H), 6.38 (d, J = 8.1 Hz, 1H), 4.56-4.52 (m, 1H), 3.91-3.86 (m, 1H), 3.77-3.72 (m, 1H), 3.53 (s, 3H), 3.32 (s, 3H), 3.02 (s, 6H), 2.56-2.50 (m, 1H), 1.58-1.50 (m, 1H), 1.33 (s, 3H), 1.27 (s, 3H), 1.06 (s, 3H). | LCMS (ES, m/z): 550 [M + H]+, Rt 0.656 min. | Column: CHIRALPAK ID, 2 × 25 cm, 5 um; Mobile Phase A: Hex: DCM = 3:1 (0.5% 2 M NH3— MeOH)--HPLC, Mobile Phase B: EtOH--HPLC(30% to 30% in 13 min); Flow rate: 20 mL/min; Wave Length: 220/254 nm; RT1(min): 10.32; RT2(min): 12.56 |
| 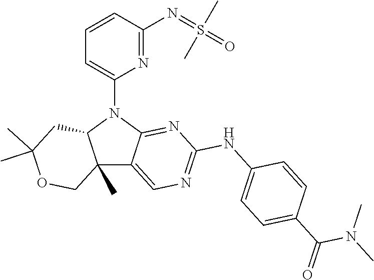 | | 1H NMR (300 MHz, DMSO-d6) δ 9.55 (s, 1H), 8.12-8.06 (m, 2H), 7.83 (d, J = 8.4 Hz, 2H), 7.59 (t, J = 8.1 Hz, 1H), 7.36 (d, J = 8.1 Hz, 2H), 6.35 (d, J = 7.5 Hz, 1H), 4.59-4.54 (m, 1H), 3.87-3.81 (m, 1H), 3.68-3.62 (m, 1H), 3.50 (s, 3H), 3.31 (s, 3H), 2.99 (s, 6H), 2.50- 2.44 (m, 1H), 1.58-1.51 (m, 1H), 1.29 (s, 3H), 1.24 (s, 3H), 1.03 (s, 3H). | LCMS (ES, m/z): 550 [M + H]+, Rt 0.652 min. | |
| 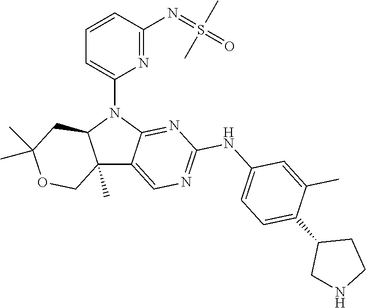 [SMILES: Cc1cc(Nc2ncc3c(n2)N(c2cccc(N=S(C)(C)=O)n2)[C@@H]2CC(C)(C)OC[C@]32C)ccc1[C@@H]1CCNC1] | 143, 144 | 1H NMR (300 MHz, DMSO-d6) δ 9.21 (s, 1H), 8.09 (d, J = 7.8 Hz, 1H), 8.01 (s, 1H), 7.70- 7.49 (m, 2H), 7.49-7.25 (m, 1H), 7.14 (d, J = 8.7 Hz, 1H), 6.32 (d, J = 7.8 Hz,1H), 4.65-4.35 (m, 1H), 3.93 -3.78 (m, 1H), 3.75 -3.60 (m,1H), 3.49 (s, 3H), 3.29-3.27 (m, 3H), 3.25- 3.09 (m, 2H), 3.01-2.82 (m, 2H), 2.65-2.55 (m, 1H), 2.31-2.27 (m, 4H), 2.11-2.01 (m, 1H), 1.72-1.46 (m, 2H), 1.27 (s, 3H), 1.22 (s, 3H), 1.02 (s, 3H). | LCMS (ES, m/z): 562 [M + H]+. Rt 1.200 min. | Column: CHIRAL ART Amylose-SA, 2*25 cm, 5 um; Mobile Phase A: Hex(0.1% 2 M NH3-MeOH) -- HPLC, Mobile Phase B: EtOH -- HPLC; Flow rate: 20 mL/min; Gradient: 20% B to 20% B in 25 min; Wave Length: 220/254 nm; RT1(min): 14.296; RT2(min): 19.437; |
| 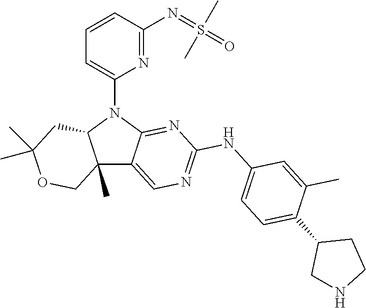 [SMILES: Cc1cc(Nc2ncc3c(n2)N(c2cccc(N=S(C)(C)=O)n2)[C@H]2CC(C)(C)OC[C@@]32C)ccc1[C@@H]1CCNC1] | | 1H NMR (300 MHz, DMSO-d6) δ 9.28-9.11 (m, 1H), 8.09 (d, J = 7.8 Hz, 1H), 8.01 (s, 1H), 7.70-7.50 (m, 2H), 7.49-7.29 (m, 1H), 7.14 (d, J = 8.7 Hz, 1H), 6.32 (d, J = 7.8 Hz, 1H), 4.60-4.32 (m, 1H), 3.93-3.75 (m, 1H), 3.73 - 3.63 (m, 1H), 3.49 (s, 3H), 3.29-3.27 (m, 3H), 3.27-3.11 (m, 2H), 3.01-2.85 (m, 2H), 2.65- 2.57 (m, 1H), 2.31-2.26 (m, 4H), 2.11-2.01 (m, 1H), 1.78-1.59 (m, 1H), 1.59-1.40 (m, 1H), 1.27 (s, 3H), 1.23 (s, 3H), 1.02 (s, 3H). | LCMS (ES, m/z): 562 [M + H]+. Rt 0.629 min. | |
| 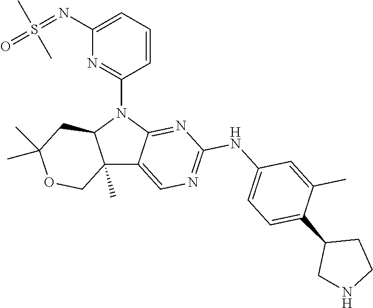 [SMILES: Cc1cc(Nc2ncc3c(n2)N(c2cccc(N=S(C)(C)=O)n2)[C@@H]2CC(C)(C)OC[C@]32C)ccc1[C@H]1CCNC1] | 149, 150 | 1H NMR (300 MHz, DMSO-d6) δ 9.25-9.14 (m, 1H), 8.11 (d, J = 8.1 Hz, 1H), 8.02 (s, 1H), 7.66-7.49 (m, 2H), 7.42 (d, J = 8.7 Hz, 1H), 7.15 (d, J = 8.4 Hz, 1H), 6.34 (d, J = 7.8 Hz, 1H), 4.59-4.49 (m, 1H), 3.88- 3.60 (m, 2H), 3.50 (s, 3H), 3.30-3.28 (m, 3H), 3.28-3.10 (m, 2H), 3.03-2.85 (m, 2H), 2.67-2.56 (m, 1H), 2.47-2.40 (m, 1H), 2.31 (s, 3H), 2.15-2.00 (m, 2H), 1.72-1.60 (m, 1H), 1.59-1.45 (m, 1H), 1.28 (s, 3H), 1.24 (s, 3H), 1.03 (s, 3H). | LCMS (ES, m/z): 562 [M + H]+, Rt 0.592 min. | Column: CHIRALPAK IG, 2 × 25 cm, 5 um; Mobile Phase A: Hex: DCM = 5:1 (0.5% 2 M NH3— MeOH)--HPLC, Mobile Phase B: EtOH--HPLC; Flow rate: 20 mL/min; Gradient: 30% B to 30% B in 23 min; Wave Length: 220/254 nm; RT1(min): 15.323; RT2(min): 19.503 |
| 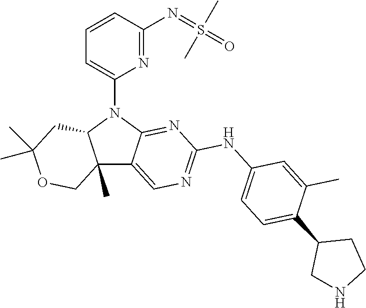 [SMILES: Cc1cc(Nc2ncc3c(n2)N(c2cccc(N=S(C)(C)=O)n2)[C@H]2CC(C)(C)OC[C@@]32C)ccc1[C@H]1CCNC1] | | 1H NMR (300 MHz, DMSO-d6) δ 9.25-9.14 (m, 1H), 8.11 (d, J = 8.1 Hz, 1H), 8.02 (s, 1H), 7.66-7.49 (m, 2H), 7.42 (d, J = 9.0 Hz, 1H), 7.15 (d, J = 8.1 Hz, 1H), 6.34 (d, J = 7.8 Hz, 1H), 4.59-4.49 (m, 1H), 3.88- 3.60 (m, 2H), 3.50 (s, 3H), 3.30 (s, 3H), 3.28-3.10 (m, 2H), 3.03-2.85 (m, 2H), 2.67-2.56 (m, 1H), 2.47- 2.40 (m, 1H), 2.31 (s, 3H), 2.15-2.00 (m, 2H), 1.70-1.60 (m, 1H), 1.59-1.45 (m, 1H), 1.28 (s, 3H), 1.24 (s, 3H), 1.03 (s, 3H). | LCMS (ES, m/z): 562 [M + H]+, Rt 0.600 min. | |
| 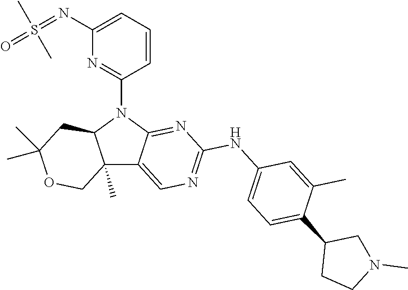 [SMILES: Cc1cc(Nc2ncc3c(n2)N(c2cccc(N=S(C)(C)=O)n2)[C@@H]2CC(C)(C)OC[C@]32C)ccc1[C@@H]1CCN(C)C1] | 145, 146 | 1H NMR (300 MHz, DMSO-d6) δ 9.16 (s, 1H), 8.09 (d, J = 7.8 Hz, 1H), 8.01 (s, 1H), 7.70- 7.50 (m, 2H), 7.48-7.40 (m, 1H), 7.20 (d, J = 8.1 Hz, 1H), 6.32 (d, J = 7.5 Hz, 1H), 4.55- 4.32 (m, 1H), 3.99-3.75(m, 1H), 3.75-3.60 (m, 1H), 3.55-3.45 (m, 4H), 3.29 (s, 3H), 2.85-2.78 (m, 1H), 2.65-2.63(m, 1H), 2.41- 2.39 (m, 2H), 2.31-2.25 (m, 8H), 1.80-1.45 (m, 2H), 1.26 (s, 3H), 1.21 (s, 3H), 1.02 (s, 3H). | LCMS (ES) m/z): 576 [M + H]+. Rt 0.599 min. | Column: CHIRAL ART Amylose-SA, 2*25 cm, 5 um; Mobile Phase A: Hex(0.1% 2 M NH3—MeOH)-- HPLC, Mobile Phase B: EtOH-- HPLC; Flow rate: 20 mL/min; Gradient: 20% B to 20% B in 25 min; Wave Length: 220/254 nm; RT1(min): 14.296; RT2(min): 19.437; Sample Solvent: EtOH-- HPLC; Injection Volume: 1 mL; Number Of Runs: 8 |
| 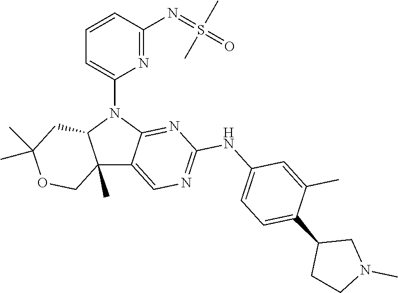 | | 1H NMR (300 MHz, DMSO-d6) δ 9.16 (s, 1H), 8.09 (d, J = 7.8 Hz, 1H), 8.01 (s, 1H), 7.65- 7.50 (m, 2H), 7.49-7.32 (m, 1H), 7.20 (d, J = 8.1 Hz, 1H), 6.32 (d, J = 7.5 Hz, 1H), 4.56- 4.32 (m, 1H), 3.95-3.75 (m, 1H), 3.75-3.60 (m, 1H), 3.55-3.45 (m, 4H), 3.29 (s, 3H), 2.85-2.75 (m, 1H), 2.65-2.61 (m, 1H), 2.41- 2.39 (m, 2H), 2.31-2.19 (m, 8H), 1.79-1.48 (m, 2H), 1.27 (s, 3H), 1.21 (s, 3H), 1.02 (s, 3H). | LCMS (ES) m/z): 576 [M + H]+. Rt 0.592 min. | |
| 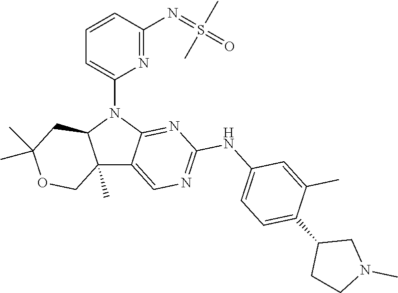 | 147, 148 | 1H NMR (300 MHz, DMSO-d6) δ 9.17 (s, 1H), 8.11 (d, J = 8.1 Hz, 1H), 8.02 (s, 1H), 7.62- 7.48 (m, 2H), 7.41 (d, J = 8.4 Hz, 1H), 7.21 (d, J = 8.4 Hz, 1H), 6.34 (d, J = 7.8 Hz, 1H), 4.60- 4.49 (m, 1H), 3.90-3.60 (m, 2H), 3.53-3.41 (m, 4H), 3.30 (s, 3H), 2.85-2.75 (m, 1H), 2.68-2.56 (m, 2H), 2.46-2.37 (m, 2H), 2.33- 2.19 (m, 7H), 1.77-1.64 (m, 1H), 1.57-1.45 (m, 1H), 1.30 (s, 3H), 1.23 (s, 3H), 1.02 (s, 3H). | LCMS (ES) m/z): 576 [M + H]+, Rt 1.317 min | Column: CHIRALPAK IG, 2 × 25 cm, 5 um; Mobile Phase A: Hex: DCM = 5:1 (0.5% 2 M NH3— MeOH)--HPLC, Mobile Phase B: EtOH--HPLC; Flow rate: 20 mL/min; Gradient: 30% B to 30% B in 23 min; Wave Length: 220/254 nm; RT1(min): 15.323; RT2(min): 19.503 |
| 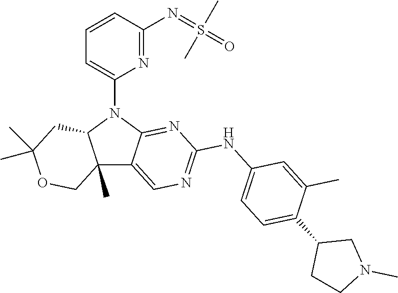 | | 1H NMR (300 MHz, DMSO-d6) δ 9.17 (s, 1H), 8.11 (d, J = 8.4 Hz, 1H), 8.02 (s, 1H), 7.62- 7.48 (m, 2H), 7.41 (d, J = 8.1 Hz, 1H), 7.21 (d, J = 8.7 Hz, 1H), 6.34 (d, J = 7.8 Hz, 1H), 4.59- 4.49 (m, 1H), 3.90-3.60 (m, 2H), 3.53-3.41 (m, 4H), 3.30 (s, 3H), 2.85-2.75 (m, 1H), 2.67-2.56 (m, 2H), 2.47-2.36 (m, 2H), 2.35- 2.15 (m, 7H), 1.77-1.64 (m, 1H), 1.57-1.45 (m, 1H), 1.28 (s, 3H), 1.23 (s, 3H), 1.02 (s, 3H) | LCMS (ES) m/z): 576 [M + H]+, Rt 1.333 min | |
| 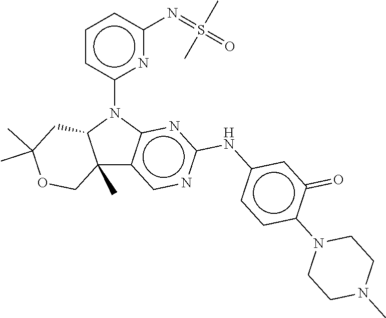 | 151, 152 | 1H NMR (300 MHz, DMSO-d6) δ 9.56 (s, 1H), 8.11 (s, 1H), 8.04 (d, J = 7.8 Hz, 1H), 7.64- 7.41 (m, 2H), 7.18-6.99 (m, 1H), 6.65-6.45 (m, 1H), 6.35 (d, J = 7.5 Hz, 1H), 4.67-4.45 (m, 2H), 3.95-3.59 (m, 2H), 3.49 (s, 3H), 3.30 (s, 3H), 2.95-2.75 (m, 2H), 2.50-2.40 (m, 1H), 2.20 (s, 3H), 2.05-1.78 (m, 4H), 1.71-1.45 (m, 3H), 1.28 (s, 3H), 1.24 (s, 3H), 1.02 (s, 3H). | LCMS (ES, m/z): 593 [M + H]+, Rt 0.563 min. | Column: CHIRALPAK IC-3, 4.6*50 mm 3 um; Mobile Phase A: (Hex: DCM= 1:1) (0.1% DEA); Mobile Phase B: EtOH = 50:50; Flow rate: 1 mL/min; Gradient: 50% B to 50% B in 10 min; Wave Length: 254/220 nm; RT1(min): 1.8; RT2(min): 4.0; |
| 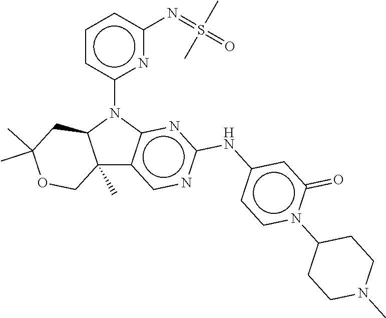 | | 1H NMR (300 MHz, DMSO-d6) δ 9.56 (s, 1H), 8.11 (s, 1H), 8.04 (d, J = 8.1 Hz, 1H), 7.64- 7.45 (m, 2H), 7.18-6.99 (m, 1H), 6.65-6.49 (m, 1H), 6.35 (d, J = 7.5 Hz, 1H), 4.70-4.45 (m, 2H), 3.95-3.61(m, 2H), 3.49 (s, 3H), 3.30 (s, 3H), 2.95-2.76 (m, 2H), 2.50-2.40 (m, 1h), 2.20 (s, 3H), 2.05-1.88 (m, 2H), 1.88-1.74 (m, 2H), 1.74-1.55 (m, 3H), 1.28 (s, 3H), 1.24(s, 3H), 1.02 (s, 3H). | LCMS (ES, m/z): 593 [M + H]+, Rt 0.566 min. | |
| 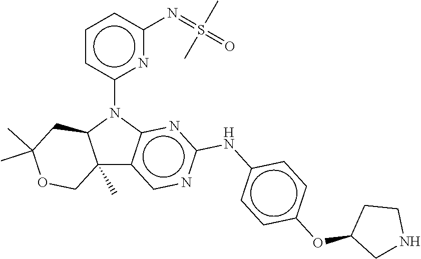 | 153, 154 | 1H NMR (300 MHz, DMSO-d6) δ 9.12 (d, J = 11.4 Hz, 1H), 8.09 (d, J = 8.1 Hz, 1H), 7.99 (s, 1H), 7.64-7.52 (m, 3H), 6.91-6.84 (m, 2H), 6.32 (d, J = 7.8 Hz, 1H), 4.95-4.82 (m, 1H), 4.56-4.51 (m, 1H), 3.85-3.81 (m, 1H), 3.67- 3.63 (m, 1H), 3.53 (s, 3H), 3.47-3.39 (m, 2H), 3.30 (s, 3H), 3.06-3.04 (m, 1H), 2.92-2.90 (m, 1H), 2.86-2.74 (m, 1H), 2.47-2.42 (m, 1H), 2.07-1.69 (m, 2H), 1.55-1.48 (m, 1H), 1.27 (s, 3H), 1.23 (s, 3H), 1.02 (s, 3H). | LCMS (ES, m/z): 564 [M + H]+, Rt 0.551 min. | Column: CHIRALPAK IG, 2*25 cm, 5 um; Mobile Phase A: MtBE(0.5% 2 M NH3—MeOH)-- HPLC, Mobile Phase B: EtOH-- HPLC; Flow rate: 20 mL/min; Gradient: 10% B to 10% B in 19 min; Wave Length: 220/254 nm; RT1(min): 11.72; RT2(min): 16.59; |
| 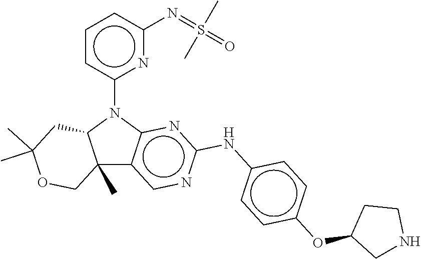 | | 1H NMR (300 MHz, DMSO-d6) δ 9.12 (d, J = 12.9 Hz, 1H), 8.09 (d, J = 7.8 Hz, 1H), 7.98 (s, 1H), 7.64-7.52 (m, 3H), 6.91-6.83 (m, 2H), 6.32 (d, J = 7.8 Hz, 1H), 4.95-4.80 (m, 1H), 4.56-4.51 (m, 1H), 3.85-3.81 (m, 1H), 3.67- 3.63 (m, 1H), 3.57 (s, 3H), 3.47-3.38 (m, 2H), 3.30 (s, 3H), 3.06-3.02 (m, 1H), 2.90- 2.87 (m, 1H), 2.82-2.74 (m, 1H), 2.44-2.42 (m, 1H), 2.07-1.69 (m, 2H), 1.55-1.47 (m, 1H), 1.27 (s, 3H), 1.23 (s, 3H), 1.02 (s, 3H). | LCMS (ES, m/z): 564 [M + H]+, Rt 0.553 min. | |
| 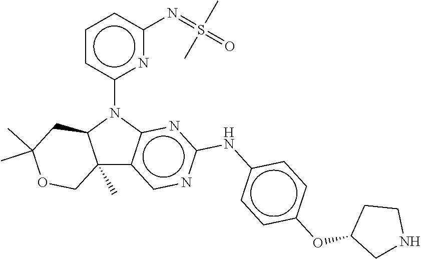 | 155, 156 | 1H NMR (300 MHz, DMSO-d6) δ 9.13 (d, J = 9.9 Hz, 1H), 8.09 (d, J = 7.8 Hz, 1H), 7.99 (s, 1H), 7.77-7.55 (m, 3H), 6.95-6.75 (m, 2H), 6.32 (d, J = 7.8 Hz, 1H), 4.95-4.81 (m, 1H), 4.65-4.55 (m, 1H), 3.85-3.80 (m, 1H), 3.76- 3.64 (m, 1H), 3.56 (s, 3H), 3.18 (s, 3H), 3.15- 3.02 (m, 2H), 3.01-2.82 (m, 2H), 2.50-2.40 (m, 1H), 2.15-1.95 (m, 2H), 1.80-1.75 (m, 1H), 1.65-1.45 (m, 1H), 1.27 (s, 3H), 1.24 (s, 3H), 1.05 (s, 3H). | LCMS (ES, m/z): 564 [M + H]+, Rt 0.567 min. | Column: CHIRALPAK ID, 2*25 cm, 5 um; Mobile Phase A: Hex(0.5% 2 M NH3—MeOH)-- HPLC, Mobile Phase B: EtOH-- HPLC; Flow rate: 20 mL/min; Gradient: 40% B to 40% B in 28.5 min; Wave Length: 220/254 nm; RT1(min): 19.50; RT2(min): 24.90; |
| 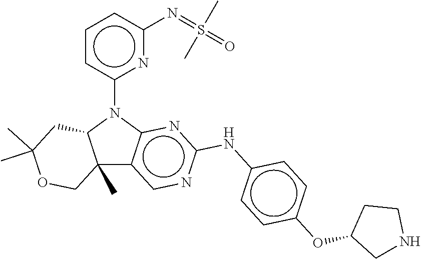 | | 1H NMR (300 MHz, DMSO-d6) δ 9.12 (d, J = 10.2 Hz, 1H), 8.12 (d, J = 7.8 Hz, 1H), 7.99 (s, 1H), 7.77-7.55 (m, 3H), 6.95-6.75 (m, 2H), 6.32 (d, J = 7.8 Hz, 1H), 4.95-4.81 (m, 1H), 4.65-4.55 (m, 1H), 3.85-3.80 (m, 1H), 3.76- 3.64 (m, 1H), 3.49 (s, 3H), 3.18 (s, 3H), 3.22- 3.02 (m, 2H), 2.98-2.77 (m, 2H), 2.50-2.40 (m, 1H), 2.17-1.95 (m, 2H), 1.85-1.75 (m, 1H), 1.66-1.41 (m, 1H), 1.27 (s, 3H), 1.24 (s, 3H), 1.03 (s, 3H). | LCMS (ES, m/z): 564 [M + H]+, Rt 0.567 min. | |
| 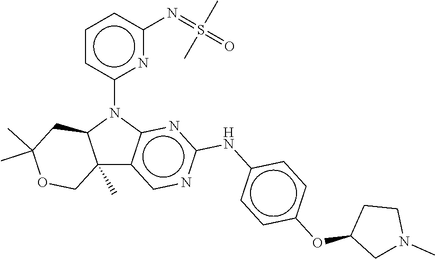 | 157, 158 | 1H NMR (300 MHz, DMSO-d6) δ 9.10 (s, 1H), 8.09 (d, J = 8.1 Hz, 1H), 7.98 (s, 1H), 7.61- 7.52 (m, 3H), 6.81 (d, J = 9.0 Hz, 2H), 6.32 (d, J = 7.8 Hz, 1H), 4.84-4.82 (m, 1H), 4.54-4.53 (m, 1H), 3.85-3.81 (m, 1H), 3.67-3.63 (m, 1H), 3.49 (s, 3H), 3.30 (s, 3H), 2.79-2.75 (m, 1H), 2.66-2.51 (m, 2H), 2.47-2.36 (m, 2H), 2.32-2.26 (m, 4H), 1.78-1.70 (m, 1H), 1.54- 1.50 (m, 1H), 1.27 (s, 3H), 1.26 (s, 3H), 1.03 (s, 3H). | LCMS (ES, m/z): 578 [M + H]+, Rt 0.559 min | Column: CHIRALPAK IG, 2*25 cm, 5 um; Mobile Phase A: MtBE(0.5% 2 M NH3—MeOH)-- HPLC, Mobile Phase B: EtOH-- HPLC; Flow rate: 20 mL/min; Gradient: 10% B to 10% B in 19 min; Wave Length: 220/254 nm RT1(min): 11.72; RT2(min): 16.59; |
| 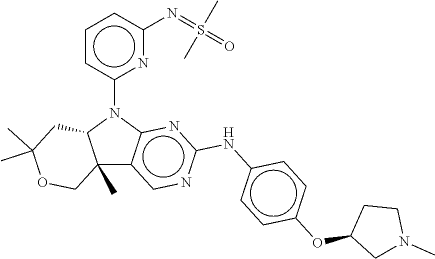 | | 1H NMR (300 MHz, DMSO-d6) δ 9.10 (s, 1H), 8.09 (d, J = 8.1 Hz, 1H), 7.98 (s, 1H), 7.61- 7.55 (m, 3H), 6.82 (d, J = 9.0 Hz, 2H), 6.32 (d, J = 8.1 Hz, 1H), 4.84-4.82 (m, 1H), 4.56-4.51 (m, 1H), 3.85-3.81 (m, 1H), 3.67-3.63 (m, 1H), 3.49 (s, 3H), 3.30 (s, 3H), 2.79-2.75 (m, 1H), 2.60-2.51 (m, 3H), 2.47-2.36 (m, 2H), 2.27 (s, 3H), 1.81-1.74 (m, 1H), 1.55-1.47 (m, 1H), 1.27 (s, 3H), 1.26 (s, 3H), 1.03 (s, 3H). | LCMS (ES, m/z): 578 [M + H]+, Rt 0.548 min | |
| 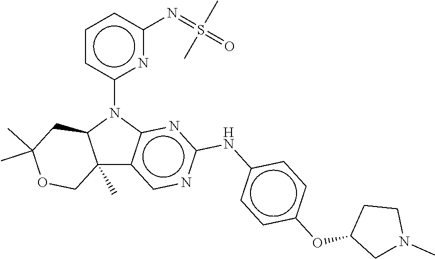 | 159, 160 | 1H NMR (300 MHz, DMSO-d6) δ 9.10 (s, 1H), 8.10 (d, J = 8.1 Hz, 1H), 7.99 (s, 1H), 7.62- 7.53 (m, 3H), 6.82 (d, J = 9.0 Hz, 2H), 6.33 (d, J = 7.5 Hz, 1H), 4.88-4.76 (m, 1H), 4.56-4.51 (m, 1H), 3.87-3.82 (m, 1H), 3.66-3.61 (m, 1H), 3.50 (s, 3H), 3.31 (s, 3H), 2.82-2.77 (m, 1H), 2.67-2.57 (m, 2H), 2.47-2.35 (m, 2H), 2.28-2.21 (m, 4H), 1.87-1.68 (m, 1H), 1.56- 1.48 (m, 1H), 1.27 (s, 3H), 1.24 (s, 3H), 1.03 (s, 3H). | LCMS (ES, m/z): 578 [M + H]+, Rt 0.555 min. | Column: CHIRALPAK ID, 2*25 cm, 5 um; Mobile Phase A: Hex(0.5% 2 M NH3—MeOH)-- HPLC, Mobile Phase B: EtOH-- HPLC; Flow rate: 20 mL/min; Gradient: 40% B to 40% B in 28.5 min; Wave Length: 220/254 nm; RT1(min): 19.50; RT2(min): 24.90; |
| 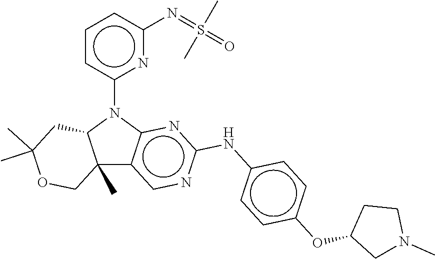 | | 1H NMR (300 MHz, DMSO-d6) δ 9.10 (s, 1H), 8.10 (d, J = 8.1 Hz, 1H), 7.98 (s, 1H), 7.62- 7.53 (m, 3H), 6.82 (d, J = 9.0 Hz, 2H), 6.32 (d, J = 7.8 Hz, 1H), 4.88-4.77 (m, 1H), 4.56-4.51 (m, 1H), 3.87-3.82 (m, 1H), 3.66-3.61 (m, 1H), 3.49 (s, 3H), 3.31 (s, 3H), 2.83-2.78 (m, 1H), 2.68-2.58 (m, 2H), 2.49-2.36 (m, 2H), 2.28-2.21 (m, 4H), 1.84-1.71 (m, 1H), 1.55- 1.48 (m, 1H), 1.27 (s, 3H), 1.24 (s, 3H), 1.03 (s, 3H). | LCMS (ES, m/z): 578 [M + H]+, Rt 0.562 min. | |
| 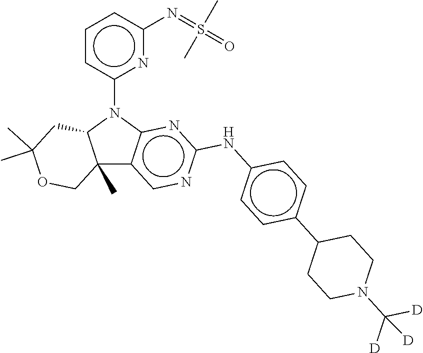 | 161 162 | 1H NMR (300 MHz, DMSO-d6) δ 9.20 (s, 1H), 8.11 (d, J = 8.1 Hz, 1H), 8.08 (s, 1H), 7.65- 7.52 (m, 3H), 7.15 (d, J = 8.4 Hz, 2H), 6.33 (d, J = 7.8 Hz, 1H), 4.55-4.50 (m, 1H), 3.87-3.82 (m, 1H), 3.66-3.61 (m, 1H), 3.48 (s, 3H), 3.29 (s, 3H), 2.87-2.81 (m 2H), 2.46-2.33 (m, 2H), 1.98-1.91 (m, 2H), 1.73-1.61 (m, 4H), 1.55- 1.47 (m, 1H), 1.27 (s, 3H), 1.22 (s, 3H), 1.03 (s, 3H). | LCMS (ES, m/z): 579 [M + H]+, Rt 0.576 min. | Column: CHIRAL ART Cellulose-SC, 2*25 cm, 5 um; Mobile Phase A: Hex: DCM = 3:1 (0.5% 2 M NH3—MeOH)-- HPLC, Mobile Phase B: EtOH-- HPLC; Flow rate: 20 mL/min; Gradient: 10% B to 10% B in 10 min; Wave Length: 220/254 nm; RT1(min): 7.66; RT2(min): 9.18 |
| 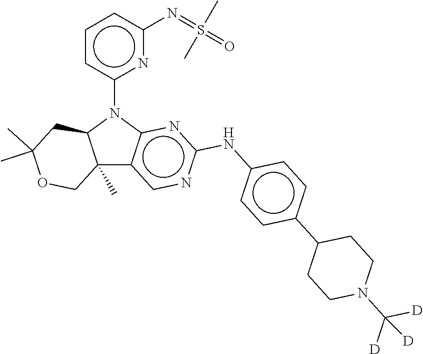 | | 1H NMR (300 MHz, DMSO-d6) δ 9.20 (s, 1H), 8.12 (d, J = 8.1 Hz, 1H), 8.00 (s, 1H), 7.65- 7.52 (m, 3H), 7.15 (d, J = 8.4 Hz, 2H), 6.33 (d, J = 7.8 Hz, 1H), 4.55-4.50 (m, 1H), 3.87-3.82 (m, 1H), 3.66-3.61 (m, 1H), 3.48 (s, 3H), 3.29 (s, 3H), 2.87-2.82 (m, 2H), 2.42-2.33 (m, 2H), 1.98-1.92 (m, 2H), 1.74-1.61 (m, 4H), 1.55- 1.47 (m, 1H), 1.27 (s, 3H), 1.22 (s, 3H), 1.01 (s, 3H). | LCMS (ES, m/z): 579 [M + H]+, Rt 0.575 min. | |
| 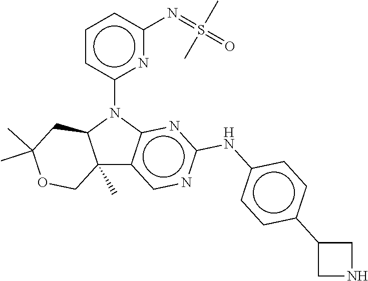 | 163, 164 | 1H NMR (300 MHz, DMSO-d6) δ 9.30 (s, 1H), 8.09 (d, J = 8.1 Hz, 1H), 8.00 (s, 1H), 7.71 (d, J = 8.4 Hz, 2H), 7.56 (t, J = 7.8 Hz, 1H), 7.28 (d, J = 7.8 Hz, 2H), 6.33 (d, J = 7.8 Hz, 1H), 4.65-4.55 (m, 1H), 4.05-3.89 (m, 2H), 3.87-3.79 (m, 3H), 3.76-3.71 (m, 2H), 3.48 (s, 3H), 3.30 (s, 3H), 2.45-2.40 (m, 1H), 1.48-1.41 (m, 1H), 1.27 (s, 3H), 1.23 (s, 3H), 1.02 (s, 3H). | LCMS (ES, m/z): 534 [M + H]+, Rt 0.575 min. | Column: CHIRALPAK IG, 2*25 cm, 5 um; Mobile Phase A: MtBE(0.5% 2 M NH3—MeOH)-- HPLC, Mobile Phase B: EtOH-- HPLC; Flow rate: 20 mL/min; Gradient: 10% B to 10% B in 22 min; Wave Length: 220/254 nm; RT1(min): 10.335; RT2(min): 16.127 |
| 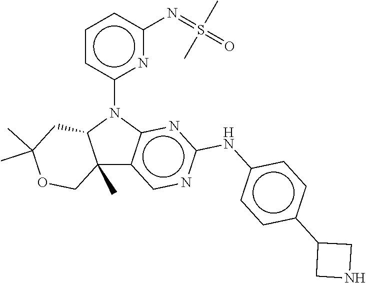 | | 1H NMR (300 MHz, DMSO-d6) δ 9.31 (s, 1H), 8.10 (d, J = 8.1 Hz, 1H), 8.02 (s, 1H), 7.72 (d, J = 8.4 Hz, 2H), 7.56 (t, J = 7.8 Hz, 1H), 7.28 (d, J = 7.8 Hz, 2H), 6.32 (d, J = 7.8 Hz, 1H), 4.65-4.55 (m, 1H), 4.25-3.98 (m, 2H), 3.87-3.78 (m, 3H), 3.68-3.61 (m, 2H), 3.48 (s, 3H), 3.29 (s, 3H), 2.50-2.42 (m, 1H), 1.56-1.42 (m, 1H), 1.27 (s, 3H), 1.23 (s, 3H), 1.02 (s, 3H). | LCMS (ES, m/z): 534 [M + H]+, Rt 0.575 min. | |
| 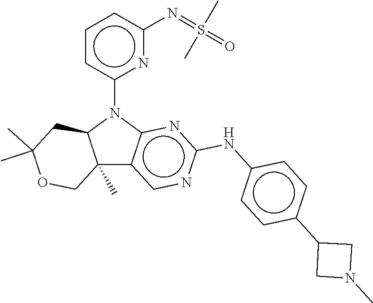 | 165, 166 | 1H NMR (300 MHz, DMSO-d6) δ 9.30 (s, 1H), 8.10 (d, J = 8.1 Hz, 1H), 8.01 (s, 1H), 7.68 (d, J = 8.4 Hz, 2H), 7.55 (t, J = 7.8 Hz, 1H), 7.22 (d, J = 8.4 Hz, 2H), 6.33 (d, J = 7.8 Hz, 1H), 4.57-4.49 (m, 1H), 3.87-3.81 (m, 1H), 3.70-3.58 (m, 3H), 3.57-3.51 (m, 1H), 3.48 (s, 3H), 3.30 (s, 3H), 3.07-3.01 (m, 2H), 2.46-2.41 (m, 1H), 2.27 (s, 3H), 1.48-1.41 (m, 1H), 1.27 (s, 3H), 1.23 (s, 3H), 1.02 (s, 3H). | LCMS (ES, m/z): 548 [M + H]+, Rt 0.584 min. | Column: CHIRALPAK IG, 2*25 cm, 5 um; Mobile Phase A: MtBE(0.5% 2 M NH3—MeOH)-- HPLC, Mobile Phase B: EtOH-- HPLC; Flow rate: 20 mL/min; Gradient: 10% B to 10% B in 22 min; Wave Length: 220/254 nm; RT1(min): 10.335; RT2(min): 16.127; |
| 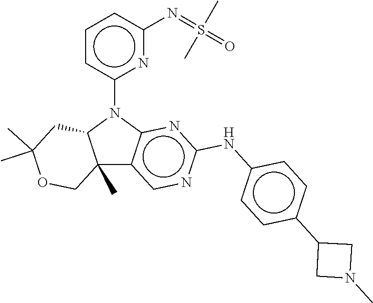 | | 1H NMR (300 MHz, DMSO-d6) δ 9.26 (s, 1H), 8.10 (d, J = 8.1 Hz, 1H), 8.01 (s, 1H), 7.68 (d, J = 8.4 Hz, 2H), 7.55 (t, J = 7.8 Hz, 1H), 7.22 (d, J = 8.4 Hz, 2H), 6.33 (d, J = 7.8 Hz, 1H), 4.57-4.49 (m, 1H), 3.87-3.81 (m, 1H), 3.70-3.58 (m, 3H), 3.57-3.51 (m, 1H), 3.48 (s, 3H), 3.30 (s, 3H), 3.07-3.01 (m, 2H), 2.46-2.41 (m, 1H), 2.28 (s, 3H), 1.48-1.41 (m, 1H), 1.27 (s, 3H), 1.23 (s, 3H), 1.02 (s, 3H). | LCMS (ES, m/z): 548 [M + H]+, Rt 0.576 min. | |
| 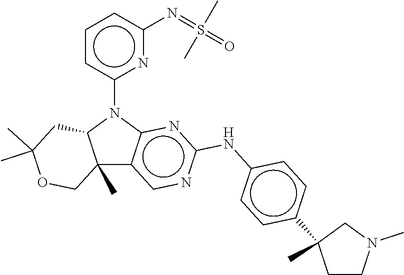 | 167, 168 | 1H NMR (300 MHz, DMSO-d6) δ 9.23 (s, 1H), 8.12 (d, J = 8.1 Hz, 1H), 8.00 (s, 1H), 7.79- 7.62 (m, 2H), 7.59-7.50 (m, 1H), 7.28-7.17 (m, 2H), 6.32 (d, J = 7.8 Hz, 1H), 4.75-4.49 (m, 1H), 3.93-3.75 (m, 1H), 3.75-3.62 (m, 1H), 3.49 (s, 3H), 3.31-3.29 (m, 3H), 2.95- 2.65 (m, 4H), 2.40-2.30 (m, 4H), 2.19-2.11 (m, 1H), 1.99-1.85 (m, 1H), 1.60-1.50 (m, 1H), 1.37 (s, 3H), 1.27 (s, 3H), 1.23 (s, 3H), 1.01 (s, 3H). | LCMS (ES, m/z): 576 [M + H]+, Rt 0.604 min. | Column: OptiChiral-C9-5, 3*25 cm, 5 um; Mobile Phase A: CO2, Mobile Phase B: MEOH(0.1% 2 M NH3— MEOH); Flow rate: 100 mL/min; Gradient: isocratic 60% B; Column Temperature (° C.): 35; Back Pressure(bar): 1000; Wave Length: 230 nm; RT1(min): 4.13; RT2(min): 6.3; |
| 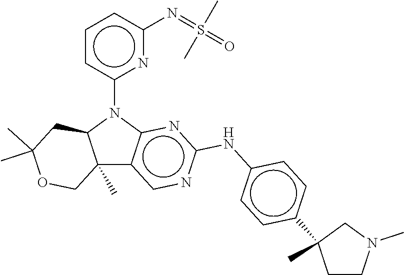 | | 1H NMR (300 MHz, DMSO-d6) δ 9.23 (s, 1H), 8.12 (d, J = 8.1 Hz, 1H), 8.00 (s, 1H), 7.66 (d, J = 8.1 Hz, 2H), 7.55 (t, J = 8.1 Hz, 1H), 7.23 (d, J = 8.7 Hz, 2H), 6.32 (d, J = 7.8 Hz, 1H), 4.75-4.49 (m, 1H), 3.93-3.75 (m, 1H), 3.75- 3.62 (m, 1H), 3.49 (s, 3H), 3.31-3.29 (m, 3H), 2.95-2.65 (m, 4H), 2.40-2.30 (m, 4H), 2.19- 2.11 (m, 1H), 1.99-1.85 (m, 1H), 1.60-1.50 (m, 1H), 1.40 (s, 3H), 1.28 (s, 3H), 1.24 (s, 3H), 1.01 (s, 3H). | LCMS (ES, m/z): 576 [M + H]+, Rt 0.609 min. | |
| 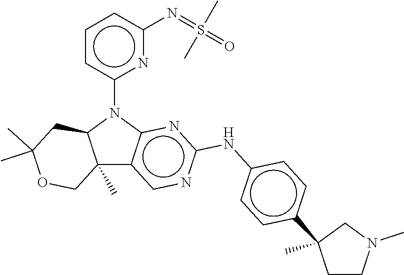 | 169, 170 | 1H NMR (300 MHz, DMSO-d6) δ 9.23 (s, 1H), 8.12 (d, J = 8.1 Hz, 1H), 8.00 (s, 1H), 7.79- 7.62 (m, 2H), 7.55 (d, J = 7.8 Hz, 1H), 7.21 (d, J = 8.4 Hz, 2H), 6.32 (d, J = 7.8 Hz, 1H), 4.75- 4.49 (m, 1H), 3.93-3.75 (m, 1H), 3.75-3.62 (m, 1H), 3.49 (s, 3H), 3.29 (s, 3H), 2.93-2.85 (m, 1H), 2.81-2.66 (m, 2H), 2.63-2.52 (m, 1H), 2.46-2.42 (m, 1H), 2.36 (s, 3H), 2.21- 2.17 (m, 1H), 1.98-1.83 (m, 1H), 1.59-1.42 (m, 1H), 1.36 (s, 3H), 1.27 (s, 3H), 1.23 (s, 3H), 1.01 (s, 3H). | LCMS (ES, m/z): 576 [M + H]+, Rt 0.605 min. | Column: CHIRALPAK ID, 2*25 cm, 5 um; Mobile Phase A: Hex: DCM = 3:1 (0.5% 2 M NH3—MeOH)-- HPLC, Mobile Phase B: EtOH-- HPLC; Flow rate: 20 mL/min; Gradient: 5% B to 5% B in 30 min; Wave Length: 220/254 nm; RT1(min): 21.24; RT2(min): 24.985; |
| 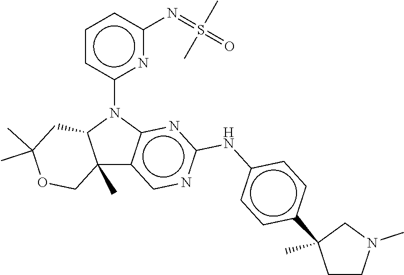 | | 1H NMR (300 MHz, DMSO-d6) δ 9.21 (s, 1H), 8.12 (d, J = 8.1 Hz, 1H), 8.00 (s, 1H), 7.79- 7.62 (m, 2H), 7.54 (d, J = 7.8 Hz, 1H), 7.21 (d, J = 8.4 Hz, 2H), 6.32 (d, J = 7.8 Hz, 1H), 4.75- 4.49 (m, 1H), 3.93-3.75 (m, 1H), 3.75-3.62 (m, 1H), 3.49 (s, 3H), 3.29 (s, 3H), 2.95-2.63 (m, 3H), 2.63-2.52 (m, 1H), 2.46-2.42 (m, 1H), 2.34 (s, 3H), 2.21-2.17 (m, 1H), 1.98- 1.83 (m, 1H), 1.59-1.42 (m, 1H), 1.36 (s, 3H), 1.27 (s, 3H), 1.23 (s, 3H), 1.01 (s, 3H). | LCMS (ES, m/z): 576 [M + H]+, Rt 0.600 min. | |
| 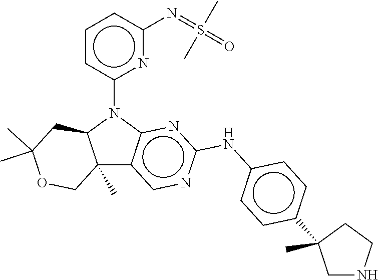 | 171, 172 | 1H NMR (300 MHz, DMSO-d6) δ 9.24 (s, 1H), 8.11 (d, J = 8.1 Hz, 1H), 8.01 (s, 1H), 7.66 (d, J = 8.7 Hz, 2H), 7.55 (t, J = 7.9 Hz, 1H), 7.23 (d, J = 8.4 Hz, 2H), 6.33 (d, J = 7.8 Hz, 1H), 4.56-4.51 (m, 1H), 3.87-3.81 (m, 1H), 3.66-3.61 (m, 1H), 3.48 (s, 3H), 3.29 (s, 3H), 3.17-2.99 (m, 3H), 2.47-2.36 (m, 2H), 2.11-1.75 (m, 2H), 1.55-1.45 (m, 1H), 1.28 (s, 3H), 1.27 (s, 3H), 1.23 (s, 3H), 1.02 (s, 3H). | LCMS (ES, m/z): 562 [M + H]+, Rt 0.567 min | Column: CHIRAL ART Cellulose-SB, 2*25 cm, 5 um; Mobile Phase A: Hex(0.5% 2 M NH3—MeOH)-- HPLC, Mobile Phase B: EtOH-- HPLC; Flow rate: 20 mL/min; Gradient: 20% B to 20% B in 19 min; Wave Length: 220/254 nm; RT1(min): 12.361; RT2(min): 15.89; |
| 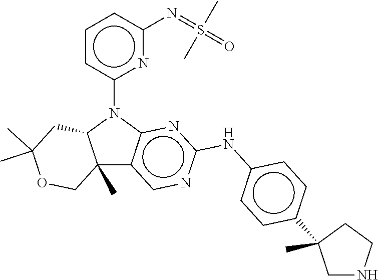 | | 1H NMR (400 MHz, DMSO-d6) δ 9.25 (s, 1H), 8.11 (d, J = 8.0 Hz, 1H), 8.01 (s, 1H), 7.66 (d, J = 8.8 Hz, 2H), 7.55 (t, J = 8.0 Hz, 1H), 7.22 (d, J = 8.4 Hz, 2H), 6.33 (d, J = 7.6 Hz, 1H), 4.55-4.51 (m, 1H), 3.87-3.81 (m, 1H), 3.66-3.61 (m, 1H), 3.49 (s, 3H), 3.29 (s, 3H), 3.17-3.04 (m, 3H), 2.50-2.43 (m, 2H), 2.09-1.90 (m, 2H), 1.54-1.49 (m, 1H), 1.28 (s, 3H), 1.27 (s, 3H), 1.23 (s, 3H), 1.02 (s, 3H). | LCMS (ES, m/z): 562 [M + H]+, Rt 0.571 min | |
| 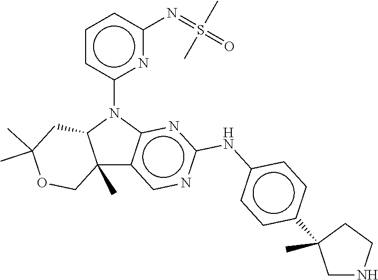 | 173, 174 | 1H NMR (400 MHz, DMSO-d6) δ 9.24 (s, 1H), 8.11 (d, J = 8.0 Hz, 1H), 8.00 (s, 1H), 7.69- 7.62 (m, 2H), 7.58-7.52 (m,1H), 7.22 (d, J = 8.4 Hz, 2H), 6.32 (d, J = 8.0 Hz, 1H), 4.56- 4.50 (m 1H), 3.85-3.61 (m, 2H), 3.48 (s, 3H), 3.29 (s, 3H), 3.11-2.90 (m, 3H), 2.49-2.41 (m, 2H), 2.12-1.94 (m, 1H), 1.92-1.82 (m, 1H), 1.56-1.46 (m, 1H), 1.27 (s, 6H), 1.22 (s, 3H), 1.01 (s, 3H). | LCMS (ES, m/z): 562 [M + H]+, Rt 0.578 min | Column: CHIRALPAK ID, 2 × 25 cm, 5 um; Mobile Phase A: Hex: DCM = 3:1 (0.5% 2 M NH3—MeOH)-- HPLC, Mobile Phase B: IPA-- HPLC; Flow rate: 20 mL/min; Gradient: 10% B to 10% B in 30 min; Wave Length: 220/254 nm; RT1(min): 10.236; RT2(min): 17.652; |
| 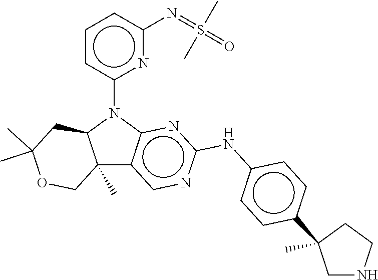 | | 1H NMR (400 MHz, DMSO-d6) δ 9.25 (s, 1H), 8.11 (d, J = 8.4 Hz, 1H), 8.01 (s, 1H), 7.71- 7.61 (m, 2H), 7.58-7.52 (m, 1H), 7.26-7.18 (m, 2H), 6.32 (d, J = 7.6 Hz, 1H), 4.57-4.50 (m, 1H), 3.86-3.61 (m, 2H), 3.48 (s, 3H), 3.29 (s, 3H), 3.13-2.94 (m, 3H), 2.49-2.41 (m, 2H), 2.12-1.96 (m, 1H), 1.95-1.85 (m, 1H), 1.56- 1.47 (m, 1H), 1.27 (s, 6H), 1.22 (s, 3H), 1.01 (s, 3H). | LCMS (ES, m/z): 562 [M + H]+, Rt 0.577 min | |
| 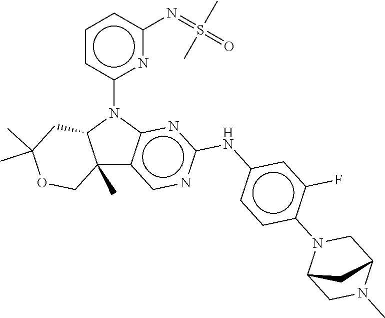 | 175, 176 | 1H NMR (300 MHz, DMSO-d6) δ 9.16 (s, 1H), 8.08 (d, J = 8.4 Hz, 1H), 7.99 (s, 1H), 7.71- 7.61 (m, 1H), 7.52 (t, J = 7.8 Hz, 1H), 7.23 (d, J = 9.6 Hz, 1H), 6.68 (t, J = 9.6 Hz, 1H), 6.32 (d, J = 7.8 Hz, 1H), 4.58-4.48 (m, 1H), 4.18- 4.12 (m, 1H), 3.87-3.60 (m, 2H), 3.48 (s, 3H), 3.45-3.35 (m, 2H), 3.29 (s, 3H), 3.23-3.14 (m, 1H), 2.80-2.60 (m, 2H), 2.49-2.39 (m, 1H), 2.27 (s, 3H), 1.86-1.67(m, 2H), 1.58-1.43 (m, 1H), 1.26 (s, 3H), 1.22 (s, 3H), 1.01 (s, 3H). | LCMS (ES, m/z): 607 [M + H]+, Rt 0.575 min. | Column: CHIRAL ART Amylose-SA, 2*25 cm, 5 um; Mobile Phase A: MtBE(0.5% IPAmine)-- HPLC, Mobile Phase B: EtOH-- HPLC; Flow rate: 20 mL/min; Gradient: 15% B to 15% B in 9.5 min; Wave Length: 220/254 nm; RT1(min): 6.64; RT2(min): 8.45 |
| 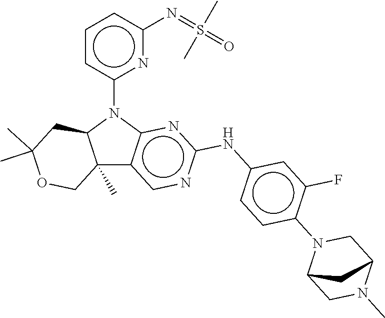 | | 1H NMR (300 MHz, DMSO-d6) δ 9.16 (s, 1H), 8.08 (d, J = 8.4 Hz, 1H), 7.99 (s, 1H), 7.68 (t, J = 9.3 Hz, 1H), 7.52 (d, J = 8.1 Hz, 1H), 7.28- 7.18 (m, 1H), 6.73-6.61 (m, 1H), 6.32 (d, J = 7.8 Hz, 1H), 4.58-4.48 (m, 1H), 4.18-4.12 (m, 1H), 3.87-3.60 (m, 2H), 3.48 (s, 3H), 3.45- 3.35 (m, 2H), 3.29 (s, 3H), 3.23-3.14 (m, 1H), 2.80-2.60 (m, 2H), 2.49-2.39 (m, 1H), 2.27 (s, 3H), 1.86-1.67(m, 2H), 1.58-1.43 (m, 1H), 1.26 (s, 3H), 1.22 (s, 3H), 1.01 (s, 3H). | LCMS (ES, m/z): 607 [M + H]+, Rt 0.583 min. | |
| 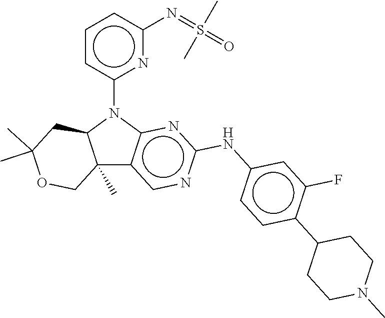 | 177, 178 | 1H NMR (300 MHz, DMSO-d6) δ 9.45 (s, 1H), 8.07-8.01 (m, 2H), 7.82-7.75 (m, 1H), 7.55 (t, J = 8.1 Hz, 1H), 7.41-7.33 (m, 1H), 7.18 (t, J = 8.7 Hz, 1H), 6.34 (d, J = 7.8 Hz, 1H), 4.58- 4.51 (m, 1H), 3.87-3.60 (m, 2H), 3.49 (s, 3H), 3.30 (s, 3H), 2.92-2.81 (m, 2H), 2.62-2.58 (m, 1H), 2.55-2.41 (m, 1H), 2.19 (s, 3H), 2.01- 1.89 (m, 2H), 1.76-1.65 (m, 4H), 161-1.48 (m, 1H), 1.26 (s, 3H), 1.22 (s, 3H), 1.02 (s, 3H). | LCMS (ES) m/z): 594 [M + H]+, Rt 0.614 min. | Column: CHIRAL ART Cellulose-SC, 2*25 cm, 5 um; Mobile Phase A: MtBE(0.5% IPAmine)-- HPLC, Mobile Phase B: IPA-- HPLC; Flow rate: 20 mL/min; Gradient: 20% B to 20% B in 9 min; Wave Length: 220/254 nm; RT1(min): 5.26; RT2(min): 7.33; |
| 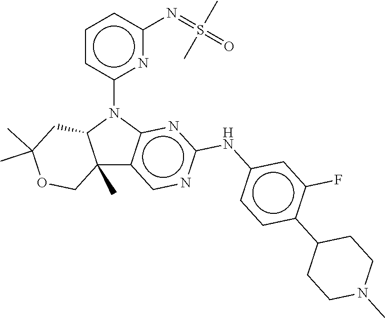 | | 1H NMR (300 MHz, DMSO-d6) δ 9.45 (s, 1H), 8.08-8.01 (m, 2H), 7.82-7.75 (m, 1H), 7.55 (t, J = 7.8 Hz, 1H), 7.41-7.33 (m, 1H), 7.18 (t, J = 8.7 Hz, 1H), 6.34 (d, J = 7.8 Hz, 1H), 4.58- 4.51 (m, 1H), 3.87-3.60 (m, 2H), 3.49 (s, 3H), 3.30 (s, 3H), 2.87-2.81 (m, 2H), 2.73-2.62 (m, 1H), 2.55-2.41 (m, 1H), 2.19 (s, 3H), 2.01- 1.89 (m, 2H), 1.78-1.67 (m, 4H), 1.65-1.58 (m, 1H), 1.27 (s, 3H), 1.22 (s, 3H), 1.01 (s, 3H). | LCMS (ES) m/z): 594 [M + H]+, Rt 0.617 min. | |
| 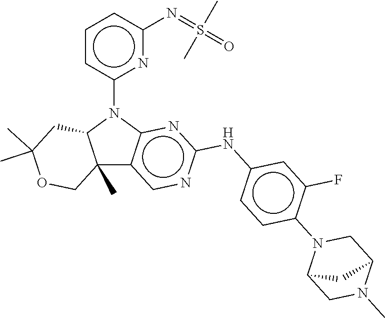 | 179, 180 | 1H NMR (300 MHz, DMSO-d6) δ 9.15 (s, 1H), 8.07 (d, J = 8.1 Hz, 1H), 7.99 (s, 1H), 7.68- 7.62 (m, 1H), 7.51 (t, J = 7.8 Hz, 1H), 7.24- 7.20 (m, 1H), 6.70-6.64 (m, 1H), 6.32 (d, J = 7.5 Hz, 1H), 4.54-4.51 (m, 1H), 4.17 (s, 1H), 3.83-3.79 (m, 1H), 3.65-3.61 (m, 1H), 3.48- 3.33 (m, 5H), 3.19 (s, 3H), 3.19-3.13 (m, 1H), 2.74-2.71 (m, 1H), 2.62-2.59 (m, 1H), 2.49- 2.42 (m, 1H), 2.26 (s, 3H), 1.85-1.64 (m, 2H), 1.56-1.45 (m, 1H), 1.26 (s, 3H), 1.22 (s, 3H), 1.01 (s, 3H). | LCMS (ES, m/z): 607 [M + H]+, Rt 0.574 min. | Column: CHIRAL ART Cellulose-SC, 2*25 cm, 5 um; Mobile Phase A: MtBE(0.5% IPAmine)-- HPLC, Mobile Phase B: IPA-- HPLC; Flow rate: 20 mL/min; Gradient: 20% B to 20% B in 10 min; Wave Length: 220/254 nm; RT1(min): 6.67; RT2(min): 8.89; |
| 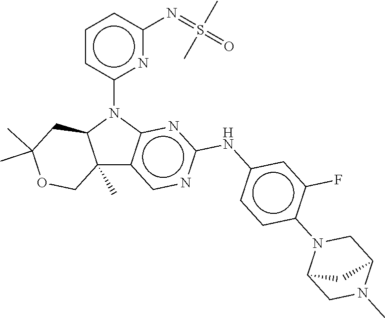 | | 1H NMR (300 MHz, DMSO-d6) δ 9.15 (s, 1H), 8.08-7.99 (m, 2H), 7.69-7.63 (m, 1H), 7.52 (t, J = 7.8 Hz, 1H), 7.25-7.21 (m, 1H), 6.72-6.66 (m, 1H), 6.32 (d, J = 7.5 Hz, 1H), 4.54-4.50 (m, 1H), 4.21-4.18 (m, 1H), 3.83-3.79 (m, 1H), 3.66-3.61 (m, 1H), 3.50-3.34 (m, 5H), 3.29 (s, 3H), 3.23-3.20 (m, 1H), 2.78-2.71 (m, 2H), 2.49-2.41 (m, 1H), 2.33 (s, 3H), 1.86- 1.81 (m, 1H), 1.78-1.75 (m, 1H), 1.52-1.47 (m, 1H), 1.26(s, 3H), 1.22 (s, 3H), 1.01 (s, 3H). | LCMS (ES, m/z): 607 [M + H]+, Rt 0.875 min. | |
| 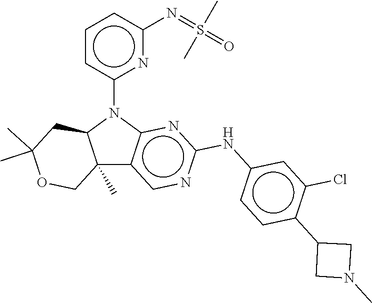 | 181, 182 | 1H NMR (400 MHz, DMSO-d6) δ 9.50 (s, 1H), 8.07-8.02 (m, 3H), 7.58-7.51 (m, 2H), 7.35 (d, J = 8.4 Hz, 1H), 6.35 (d, J = 7.6 Hz, 1H), 4.56-4.53 (m, 1H), 3.84-3.81 (m, 1H), 3.75- 3.71 (m, 1H), 3.69-3.63 (m, 3H), 3.50 (s, 3H), 3.29 (s, 3H), 3.02-2.99 (m, 2H), 2.46-2.41 (m, 1H), 2.23 (s, 3H), 1.55-1.50 (m, 1H), 1.27 (s, 3H), 1.22 (s, 3H), 1.01 (s, 3H). | LCMS (ES, m/z): 582, 584 [M + H]+, Rt 0.589 min. | Column: CHIRAL ART Cellulose-SB, 2*25 cm, 5 um; Mobile Phase A: Hex(0.5% IPAmine)-- HPLC, Mobile Phase B: EtOH-- HPLC; Flow rate: 20 mL/min; Gradient: 20% B to 20% B in 21 min; Wave Length: 220/254 nm; RT1(min): 14.681; RT2(min): 18.448; |
| 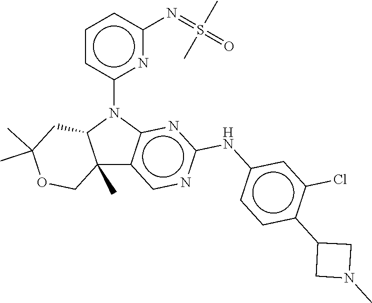 | | 1H NMR (400 MHz, DMSO-d6) δ 9.50 (s, 1H), 8.07-8.03 (m, 3H), 7.58-7.52 (m, 2H), 7.35 (d, J = 8.8 Hz, 1H), 6.35 (d, J = 7.6 Hz, 1H), 4.57-4.53 (m, 1H), 3.84-3.81 (m, 1H), 3.76- 3.63 (m, 4H), 3.50 (s, 3H), 3.29 (s, 3H), 3.04- 3.01 (m, 2H), 2.46-2.41 (m, 1H), 2.24 (s, 3H), 1.55-1.50 (m, 1H), 1.27 (s, 3H), 1.22 (s, 3H), 1.01 (s, 3H). | LCMS (ES, m/z): 582, 584 [M + H]+, Rt 0.590 min. | |
| 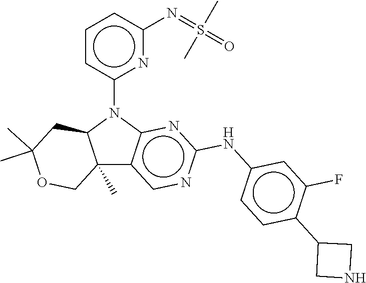 | 183, 189 | 1H NMR (400 MHz, DMSO-d6) δ 9.49 (s, 1H), 8.07-8.02 (m, 2H), 7.80-7.77 (m, 1H), 7.55 (t, J = 8.0 Hz, 1H), 7.44-7.31 (m, 2H), 6.35 (d, J = 8.0 Hz, 1H), 4.56-4.52 (m, 1H), 3.99-3.95 (m, 2H), 3.84-3.63 (m, 4H), 3.48 (s, 3H), 3.32- 3.28 (m, 5H), 2.46-2.42 (m, 1H), 1.56-1.50 (m, 1H), 1.27 (s, 3H), 1.22 (s, 3H), 1.01 (s, 3H). | LCMS (ES, m/z): 552 [M + H]+, Rt 2.377 min. | Column: CHIRALPAK IF, 2*25 cm, 5 um; Mobile Phase A: MtBE(0.5% IPAmine)-- HPLC, Mobile Phase B: EtOH-- HPLC; Flow rate: 20 mL/min; Gradient: 5% B to 5% B in 10 min; Wave Length: 220/254 nm; RT1(min): 7.8; RT2(min): 9.263; |
| 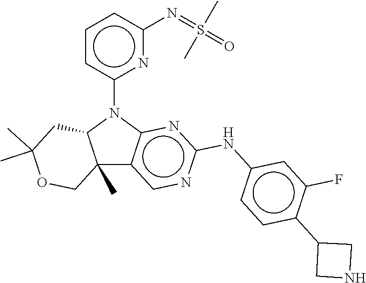 | | 1H NMR (400 MHz, DMSO-d6) δ 8.08 (s, 1H), 7.89 (d, J = 8.0 Hz, 1H), 7.78-7.75 (m, 1H), 7.61-7.57 (m, 1H), 7.49-7.41 (m, 2H), 6.45 (d, J = 8.0 Hz, 1H), 4.66-4.63 (m, 1H), 4.27- 4.23 (m, 3H), 4.20-4.17 (m, 2H), 3.85-3.82 (m, 1H), 3.68-3.64 (m, 1H), 3.47 (s, 3H), 3.31 (s, 3H), 2.41-2.37 (m, 1H), 1.56-1.51 (m, 1H), 1.32 (s, 3H), 1.21 (s, 3H), 1.01 (s, 3H). | LCMS (ES, m/z): 552 [M + H]+, Rt 1.079 min. | |
| 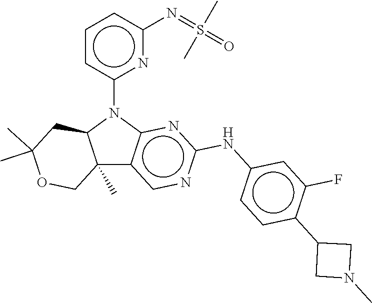 | 184, 185 | 1H NMR (400 MHz, DMSO-d6) δ 9.49 (s, 1H), 8.07-8.06 (m, 2H), 7.83-7.78 (m, 1H), 7.57 (t, J = 8.0 Hz, 1H), 7.43 (d, J = 8.4 Hz, 1H), 7.33 (t, J = 8.8 Hz, 1H), 6.35 (d, J = 7.6 Hz, 1H), 4.56-4.53 (m, 1H), 3.85-3.82 (m, 1H), 3.70- 3.61 (m, 4H), 3.49 (s, 3H), 3.30 (s, 3H), 3.04 (t, J = 6.0 Hz, 2H), 2.47-2.42 (m, 1H), 2.25(s, 3H), 1.56-1.50 (m, 1H), 1.28 (s, 3H), 1.22 (s, 3H), 1.01 (s, 3H). | LCMS (ES, m/z): 566 [M + H]+, Rt 1.117 min. | Column: CHIRALPAK IF, 2*25 cm, 5 um; Mobile Phase A: MtBE(0.5% IPAmine)-- HPLC, Mobile Phase B: EtOH--HPLC; Flow rate: 20 mL/min; Gradient: 5% B to 5% B in 10 min; Wave Length: 220/254 nm; RT1(min): 7.8; RT2(min): 9.263; |
| 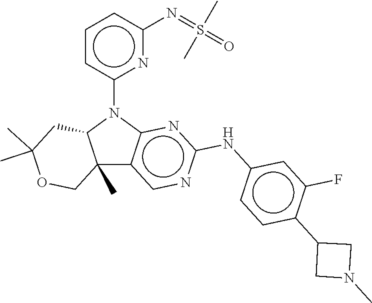 | | 1H NMR (400 MHz, DMSO-d6) δ 9.49 (s, 1H), 8.07-8.06 (m, 2H), 7.82-7.78 (m, 1H), 7.57 (t, J = 8.0 Hz, 1H), 7.43 (d, J = 8.4 Hz, 1H), 7.30 (t, J = 8.8 Hz, 1H), 6.35 (d, J = 6.8 Hz, 1H), 4.56-4.53 (m, 1H), 3.85-3.82 (m, 1H), 3.70- 3.61 (m, 4H), 3.49 (s, 3H), 3.30 (s, 3H), 3.05 (t, J = 6.0 Hz, 2H), 2.47-2.42 (m, 1H), 2.25 (s, 3H), 1.56-1.50 (m, 1H), 1.28 (s, 3H), 1.23 (s, 3H), 1.01 (s, 3H). | LCMS (ES, m/z): 566 [M + H]+, Rt 0.556 min. | |
| 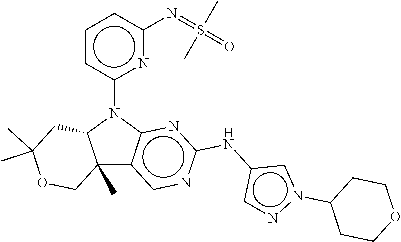 | 186, 194 | 1H NMR (400 MHz, DMSO-d6) δ 9.11 (s, 1H), 7.97-7.93 (m, 3H), 7.55-7.47 (m, 2H), 6.33 (d, J = 8.0 Hz, 1H), 4.52-4.49 (m, 1H), 4.33- 4.29 (m, 1H), 3.96-3.94 (m, 2H), 3.82-3.79 (m, 1H), 3.64-3.61 (m, 1H), 3.50-3.43 (m, 5H), 3.29 (s, 3H), 2.43-2.41 (m, 1H), 1.97- 1.86 (m, 4H), 1.53-1.47 (m, 1H), 1.25 (s, 3H), 1.22 (s, 3H), 1.01 (s, 3H). | LCMS (ES, m/z): 553 [M + H]+, Rt 0.609 min. | Column: CHIRALPAK IF, 2*25 cm, 5 um; Mobile Phase A: MtBE(0.5% IPAmine) -- HPLC, Mobile Phase B: EtOH-- HPLC; Flow rate: 20 mL/min; Gradient: 5% B to 5% B in 10 min; Wave Length: 220/254 nm; RT1(min): 7.8; RT2(min): 9.263; |
| 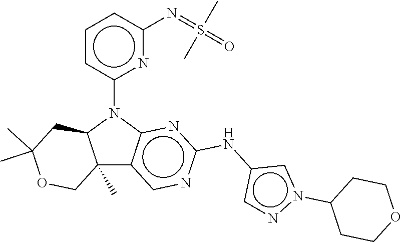 | | 1H NMR (300 MHz, DMSO-d6) δ 9.11 (s, 1H), 8.01-7.92 (m, 3H), 7.56-7.47 (m, 2H), 6.33 (d, J = 7.8 Hz, 1H), 4.51-4.48 (m, 1H), 4.33- 4.31 (m, 1H), 3.97-3.93 (m, 2H), 3.82-3.78 (m, 1H), 3.65-3.61 (m, 1H), 3.47-3.43 (m, 4H), 3.41-3.78 (m, 1H), 3.20 (s, 3H), 2.35- 2.27 (m, 1H), 1.98-1.86 (m, 4H), 1.53-1.45 (m, 1H), 1.25 (s, 3H), 1.21 (s, 3H), 1.01 (s, 3H). | LCMS (ES, m/z): 553 [M + H]+, Rt 0.614 min. | |
| 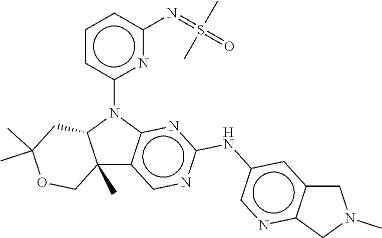 | 187, 188 | 1H NMR (400 MHz, DMSO-d6) δ 9.46 (s, 1H), 8.54 (s, 1H), 8.15-8.00 (m, 3H), 7.54 (t, J = 8.0 Hz, 1H), 6.34 (d, J = 7.6 Hz, 1H), 4.56- 4.53 (m, 1H), 3.87-3.82 (m, 3H), 3.76-3.71 (m, 2H), 3.66-3.61 (m, 1H), 3.48 (s, 3H), 3.30 (s, 3H), 2.50 (s, 3H), 2.46-2.42 (m, 1H), 1.55- 1.50 (m, 1H), 1.27 (s, 3H), 1.22 (s, 3H), 1.01 (s, 3H). | LCMS (ES, m/z): 535 [M + H]+, Rt 0.518 min. | Column: CHIRALPAK IE, 2*25 cm, 5 um; Mobile Phase A: MtBE(0.5% IPAmine) -- HPLC-- [symbol] , Mobile Phase B: EtOH--HPLC; Flow rate: 20 mL/min; Gradient: 15% B to 15% B in 13 min; Wave Length: 220/254 nm; RT1(min): 6.37; RT2(min): 8.309; |
| 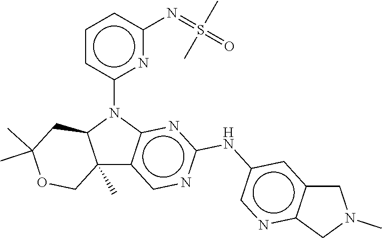 | | 1H NMR (400 MHz, DMSO-d6) δ 9.45 (s, 1H), 8.54 (m, 1H), 8.15-8.00 (m, 3H), 7.53 (t, J = 8.0 Hz, 1H), 6.34 (d, J = 8.0 Hz, 1H), 4.56- 4.53 (m, 1H), 3.87-3.82 (m, 3H), 3.76-3.71 (m, 2H), 3.66-3.62 (m, 1H), 3.48 (s, 3H), 3.30 (s, 3H), 2.55 (s, 3H), 2.46-2.42 (m, 1H), 1.55- 1.49 (m, 1H), 1.27 (s, 3H), 1.22 (s, 3H), 1.01 (s, 3H). | LCMS (ES, m/z): 535 [M + H]+, Rt 0.517 min. | |
| 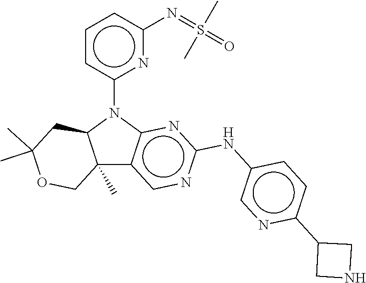 | 190 191 | 1H NMR (400 MHz, DMSO-d6) δ 9.44 (s, 1H), 8.81 (s, 1H), 8.18 (d, J = 8.4 Hz, 1H), 8.08- 8.04 (m, 2H), 7.55 (t, J = 8.0 Hz, 1H), 7.26 (d, J = 8.4 Hz, 1H), 6.35 (d, J = 7.6 Hz, 1H), 4.56- 4.53 (m, 1H), 3.95-3.89 (m, 1H), 3.85-3.79 (m, 3H), 3.70-3.64 (m, 3H), 3.49 (s, 3H), 3.30 (s, 3H), 2.47-2.42 (m, 1H), 1.55-1.49 (m, 1H), 1.27 (s, 3H), 1.23 (s, 3H), 1.01 (s, 3H). | LCMS (ES, m/z): 535 [M + H]+, Rt 0.510 min. | Column: CHIRAL ART Cellulose-SB, 2*25 cm, 5 um; Mobile Phase A: Hex(0.5% 2 M NH3—MeOH) -- HPLC, Mobile Phase B: EtOH-- HPLC; Flow rate: 20 mL/min; Gradient: 30% B to 30% B in 15 min; Wave Length: 220/254 nm; RT1(min): 10.08; RT2(min): 13.01; |
| 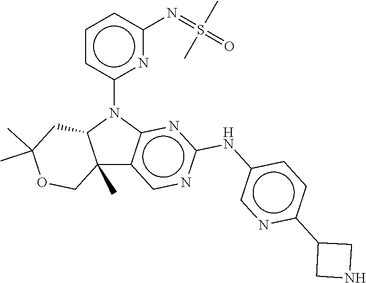 | | 1H NMR (400 MHz, DMSO-d6) δ 9.44 (s, 1H), 8.81 (s, 1H), 8.18 (d, J = 9.6 Hz, 1H), 8.08- 8.04 (m, 2H), 7.55 (t, J = 8.0 Hz, 1H), 7.26 (d, J = 8.4 Hz, 1H), 6.35 (d, J = 7.6 Hz, 1H), 4.56- 4.53 (m, 1H), 3.95-3.79 (m, 4H), 3.70-3.64 (m, 3H), 3.49 (s, 3H), 3.30 (s, 3H), 2.47-2.42 (m, 1H), 1.55-1.49 (m, 1H), 1.27 (s, 3H), 1.23 (s, 3H), 1.01 (s, 3H). | LCMS (ES, m/z): 535 [M + H]+, Rt 0.510 min. | |
| 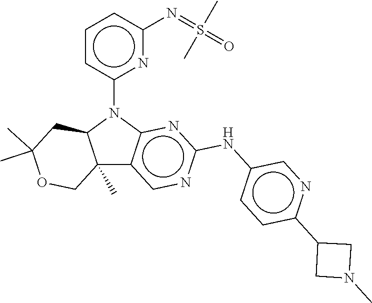 | 192, 193 | 1H NMR (400 MHz, DMSO-d6) δ 9.43 (s, 1H), 8.78 (s, 1H), 8.16-8.03 (m, 3H), 7.55 (t, J = 8.0 Hz, 1H), 7.26 (d, J = 8.4 Hz, 1H), 6.34 (d, J = 8.0 Hz, 1H), 4.56-4.52 (m, 1H), 3.85-3.82 (m, 1H), 3.66-3.57 (m, 4H), 3.48 (s, 3H), 3.29 (s, 3H), 3.22-3.19 (m, 2H), 2.47-2.42 (m, 1H), 2.26 (s, 3H), 1.55-1.49 (m, 1H), 1.27 (s, 3H), 1.22 (s, 3H), 1.01 (s, 3H). | LCMS (ES, m/z): 549 [M + H]+, Rt 0.527 min. | Column: CHIRAL ART Cellulose-SB, 2*25 cm, 5 um; Mobile Phase A: Hex(0.5% 2 M NH3-MeOH) -- HPLC, Mobile Phase B: EtOH- -HPLC; Flow rate: 20 mL/min; Gradient: 30% B to 30% B in 15 min; Wave Length: 220/254 nm; RT1(min): 10.08; RT2(min): 13.01; |
| 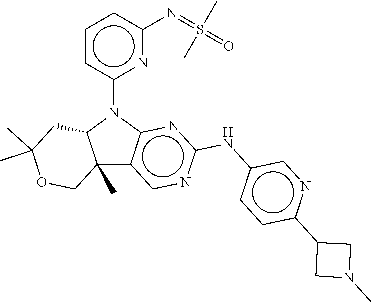 | | 1H NMR (400 MHz, DMSO-d6) δ 9.43 (s, 1H), 8.79 (s, 1H), 8.17-8.03 (m, 3H), 7.55 (t, J = 8.0 Hz, 1H), 7.26 (d, J = 8.4 Hz, 1H), 6.34 (d, J = 7.6 Hz, 1H), 4.56-4.52 (m, 1H), 3.85-3.82 (m, 1H), 3.66-3.57 (m, 4H), 3.48 (s, 3H), 3.29 (s, 3H), 3.22-3.19 (m, 2H), 2.47-2.42 (m, 1H), 2.26 (s, 3H), 1.55-1.49 (m, 1H), 1.27 (s, 3H), 1.23 (s, 3H), 1.01 (s, 3H). | LCMS (ES, m/z): 549 [M + H]+, Rt 0.519 min. | |
| 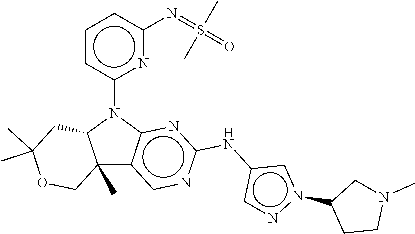 | 195, 196 | 1H NMR (400 MHz, DMSO-d6) δ 9.15 (s, 1H), 7.99 (s, 1H), 7.94 (br, 1H), 7.59-7.50 (m, 1H), 7.45 (s, 1H), 6.33 (d, J = 7.8 Hz, 1H), 4.89- 4.78 (m, 1H), 4.55-4.46 (m, 1H), 3.87-3.82 (m, 1H), 3.66-3.61 (m, 1H), 3.49 (s, 3H), 3.39 (s, 3H), 2.85-2.65 (m, 4H), 2.48-2.31 (m, 3H), 2.28 (s, 3H), 2.09-1.97 (m, 1H), 1.55-1.44 (m, 1H), 1.27 (s, 3H), 1.24 (s, 3H), 1.02 (s, 3H). | LCMS (ES, m/z): 552 [M + H]+; RT: 0.978 min. | Column: CHIRAL ART Cellulose-SC, 2*25 cm, 5 um; Mobile Phase A: Hex: DCM = 3:1 (0.5% 2 M NH3—MeOH)-- HPLC, Mobile Phase B: IPA-- HPLC; Flow rate: 20 mL/min; Gradient: 20% B to 20% B in 17 min; Wave Length: 220/254 nm; RT1(min): 11.876; RT2(min): 15.277 |
| 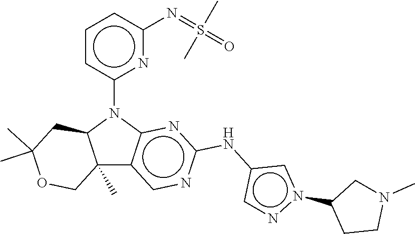 | | 1H NMR (300 MHz, DMSO-d6) δ 9.13 (s, 1H), 8.11-7.87 (m, 3H), 7.55 (t, J = 7.5 Hz, 1H), 7.45 (s, 1H), 6.32 (d, J = 7.8 Hz, 1H), 4.89- 4.76 (m, 1H), 4.56-4.45 (m, 1H), 3.87-3.82 (m, 1H), 3.66-3.61 (m, 1H), 3.48 (s, 3H), 3.30 (s, 2H), 2.87-2.66 (m, 3H), 2.48-2.31 (m, 4H), 2.28 (s, 3H), 2.11-1.94 (m, 1H), 1.56-1.42 (m, 1H), 1.27 (s, 3H), 1.24 (s, 3H), 1.01 (s, 3H). | LCMS (ES, m/z): 552 [M + H]+; RT: 0.976 min. | |
| 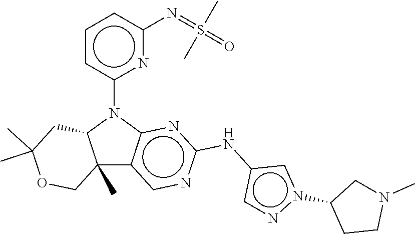 | 197, 198 | 1H NMR (300 MHz, DMSO-d6) δ 9.13 (s, 1H), 8.13-7.89 (m, 3H), 7.55 (t, J = 7.8 Hz, 1H), 7.45 (s, 1H), 6.32 (d, J = 7.8 Hz, 1H), 4.87- 4.79 (m, 1H), 4.53-4.49 (m, 1H), 3.83-3.79 (m, 1H), 3.65-3.61 (m, 1H), 3.48 (s, 3H), 3.30 (s, 3H), 2.89-2.68 (m, 3H), 2.47-2.40 (m, 2H), 2.38-2.31 (m, 1H), 2.27 (s, 3H), 2.08-1.99 (m, 1H), 1.54-1.46 (m, 1H), 1.25 (s, 3H), 1.22 (s, 3H), 1.01 (s, 3H). | LCMS (ES, m/z): 552 [M + H]+, Rt 0.515 min. | Column: CHIRAL ART Cellulose-SC, 2 × 25 cm, 5 um; Mobile Phase A: Hex: DCM = 3:1 (0.5% 2 M NH3—MeOH)-- HPLC, Mobile Phase B: IPA-- HPLC(20% to 20% in 18 min); Flow rate: 20 mL/min; Wave Length: 220/254 nm; RT1(min): 11.953; RT2(min): 16.001 |
| 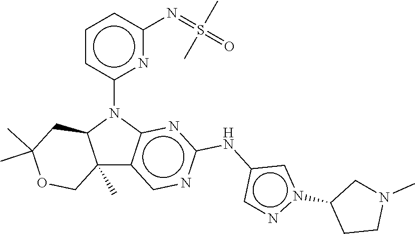 | | 1H NMR (300 MHz, DMSO-d6) δ 9.13 (s, 1H), 8.12-7.91 (m, 3H), 7.55 (t, J = 8.1 Hz, 1H), 7.45 (s, 1H), 6.32 (d, J = 7.8 Hz, 1H), 4.86- 4.80 (m, 1H), 4.53-4.48 (m, 1H), 3.83-3.79 (m, 1H), 3.65-3.61 (m, 1H), 3.48 (s, 3H), 3.30 (s, 3H), 2.84-2.78 (m, 1H), 2.73-2.67 (m, 2H), 2.47-2.41 (m, 2H), 2.39-2.30 (m, 1H), 2.27 (s, 3H), 2.04-2.02 (m, 1H), 1.54-1.46 (m, 1H), 1.25 (s, 3H), 1.22 (s, 3H), 1.01 (s, 3H). | LCMS (ES, m/z): 552 [M + H]+, Rt 0.523 min. | |
| 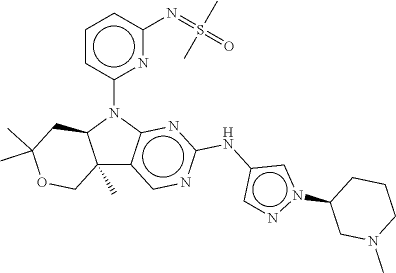 | 199, 200 | 1H NMR (300 MHz, DMSO-d6) δ 9.12 (s, 1H), 8.11-7.92 (m, 3H), 7.55 (t, J = 8.1 Hz, 1H), 7.46 (s, 1H), 6.32 (d, J = 7.8 Hz, 1H), 4.53- 4.48 (m, 1H), 4.14-4.08 (m, 1H), 3.95-3.88 (m, 1H), 3.83-3.79 (m, 1H), 3.65-3.61 (m, 1H), 3.48 (s, 3H), 3.30 (s, 3H), 2.94-2.89 (m, 1H), 2.47-2.41 (m, 2H), 2.18 (s, 3H), 2.17- 2.12 (m, 1H), 1.74-1.71 (m, 1H), 1.63-1.46 (m, 4H), 1.25 (s, 3H), 1.22 (s, 3H), 1.01 (s, 3H). | LCMS (ES, m/z): 566 [M + H]+, Rt 0.526 min. | Column: CHIRAL ART Cellulose-SB, 2 × 25 cm, 5 um; Mobile Phase A: Hex(0.5% IPAmine)-- HPLC, Mobile Phase B: EtOH-- HPLC(30% to 30% in 13 min); Flow rate: 20 mL/min; Wave Length: 220/254 nm; RT1(min): 9.112; RT2(min): 10.957 |
| 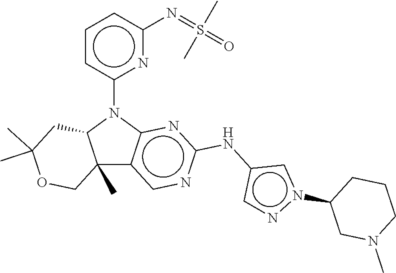 | | 1H NMR (300 MHz, DMSO-d6) δ 9.12 (s, 1H), 8.12-7.93 (m, 3H), 7.55 (t, J = 8.1 Hz, 1H), 7.46 (s, 1H), 6.32 (d, J = 7.8 Hz, 1H), 4.54- 4.49 (m, 1H), 4.14-4.08 (m, 1H), 3.96-3.89 (m, 1H), 3.83-3.79 (m, 1H), 3.65-3.61 (m, 1H), 3.48 (s, 3H), 3.30 (s, 3H), 2.90 (d, J = 7.2 Hz, 1H), 2.45-2.39 (m, 2H), 2.24-2.08 (m, 4H), 1.75-1.68 (m, 1H), 1.63-1.47 (m, 4H), 1.25 (s, 3H), 1.22 (s, 3H), 1.01 (s, 3H). | LCMS (ES, m/z): 566 [M + H]+, Rt 0.526 min. | |
| 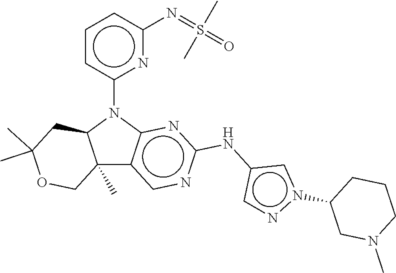 | 201, 202 | 1H NMR (300 MHz, DMSO-d6) δ 9.11 (s, 1H), 8.12-7.97 (m, 2H), 7.88 (s, 1H), 7.55 (t, J = 7.8 Hz, 1H), 7.45 (s, 1H), 6.32 (d, J = 7.8 Hz, 1H), 4.53-4.48 (m, 1H), 4.13-4.07 (m, 1H), 3.95-3.89 (m, 1H), 3.83-3.79 (m, 1H), 3.65- 3.61 (m, 1H), 3.47 (s, 3H), 3.29 (s, 3H), 2.92- 2.91 (m, 1H), 2.50-2.49 (m, 2H), 2.17 (s, 3H), 2.15-2.08 (m, 1H), 1.75-1.71 (m, 1H), 1.69- 1.67 (m, 4H), 1.25 (s, 3H), 1.22 (s, 3H), 1.01 (s, 3H). | LCMS (ES, m/z): 566 [M + H]+, Rt 0.528 min. | Column: CHIRAL ART Cellulose-SB, 2*25 cm, 5 um; Mobile Phase A: MtBE(0.5% IPAmine)-- HPLC, Mobile Phase B: EtOH-- HPLC; Flow rate: 20 mL/min; Gradient: 4% B to 4% B in 14.5 min; Wave Length: 220/254 nm; RT1(min): 9.78; RT2(min): 11.89; |
| 590 | | 1H NMR (300 MHz, DMSO-d6) δ 9.11 (s, 1H), 7.97-7.92 (m, 2H), 7.88 (s, 1H), 7.55-7.45 (m, 2H), 6.31 (d, J = 7.8 Hz, 1H), 4.52-4.48 (m, 1H), 4.14-4.08 (m, 1H), 3.95-3.79 (m, 2H), 3.65-3.61 (m, 1H), 3.47 (s, 3H), 3.29 (s, 3H), 2.94-2.91 (m, 1H), 2.50-2.40 (m, 2H), 2.17- 2.12 (m, 4H), 1.75-1.71 (m, 1H), 1.67-1.45 (m, 4H), 1.25 (s, 3H), 1.22 (s, 3H), 1.01 (s, 3H). | LCMS (ES) m/z): 566 [M + H]+, Rt 0.519 min. | |
| 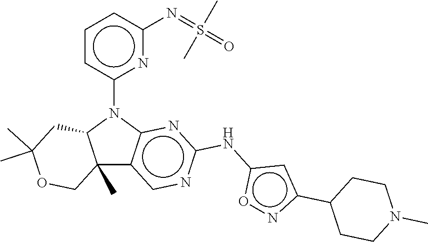 | 203, 204 | 1H NMR (300 MHz, DMSO-d6) δ 10.90 (s, 1H), 8.10 (s, 1H), 7.99 (d, J = 8.1 Hz, 1H), 7.55 (t, J = 8.1 Hz, 1H), 6.36 (d, J = 7.8 Hz, 1H), 6.19 (s, 1H), 4.59-4.55 (m, 1H), 3.87- 3.82 (m, 1H), 3.66-3.61 (m, 1H), 3.48 (s, 3H), 3.30 (s, 3H), 2.87-2.82 (m, 2H), 2.66-2.52 (m, 1H), 2.50-2.40 (m, 1H), 2.21 (s, 3H), 2.05- 1.97 (m, 2H), 1.86-1.81 (m, 2H), 1.69-1.52 (m, 3H), 1.28 (s, 3H), 1.23 (s, 3H), 1.00 (s, 3H). | LCMS (ES, m/z): 567 [M + H]+, Rt 0.555 min. | Column: CHIRAL ART Cellulose-SC, 2*25 cm, 5 um; Mobile Phase A: Hex: DCM = 3:1 (0.5% 2 M NH3—MeOH)-- HPLC, Mobile Phase B: EtOH-- HPLC; Flow rate: 20 mL/min; Gradient: 10% B to 10% B in 20 min; Wave Length: 220/254 nm; RT1(min): 15.74; RT2(min): 18.42; |
| 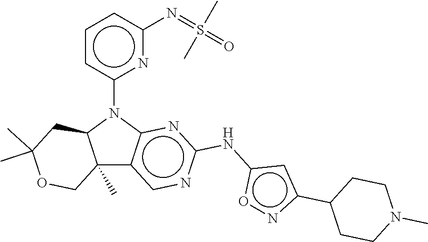 | | 1H NMR (300 MHz, DMSO-d6) δ 10.90 (s, 1H), 8.10 (s, 1H), 7.99 (d, J = 8.1 Hz, 1H), 7.55 (t, J = 8.1 Hz, 1H), 6.36 (d, J = 7.8 Hz, 1H), 6.19 (s, 1H), 4.59-4.55 (m, 1H), 3.87- 3.82 (m, 1H), 3.66-3.61 (m, 1H), 3.48 (s, 3H), 3.30 (s, 3H), 2.82-2.78 (m, 2H), 2.61-2.53 (m, 1H), 2.46-2.39 (m, 1H), 2.19 (s, 3H), 2.02- 1.95 (m, 2H), 1.89-1.78 (m, 2H), 1.69-1.51 (m, 3H), 1.28 (s, 3H), 1.22 (s, 3H), 1.00 (s, 3H). | LCMS (ES, m/z): 567 [M + H]+, Rt 0.549 min | |
| 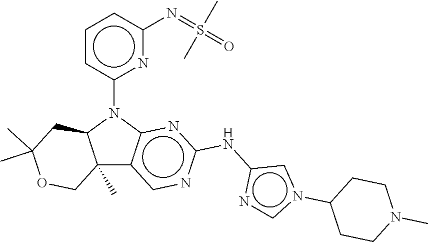 | 205, 206 | 1H NMR (300 MHz, DMSO-d6) δ 9.10 (s, 1H), 7.97-7.91 (m, 3H), 7.54 (t, J = 7.8 Hz, 1H), 7.46 (s, 1H), 6.33 (d, J = 7.8 Hz, 1H), 4.53- 4.48 (m, 1H), 4.08-4.00 (m, 1H), 3.83-3.79 (m, 1H), 3.65-3.61 (m, 1H), 3.48 (s, 3H), 3.29 (s, 3H), 2.88-2.82 (m, 2H), 2.46-2.39 (m, 1H), 2.21 (s, 3H), 2.09-1.86 (m, 6H), 1.54-1.46 (m, 1H), 1.25 (s, 3H), 1.22 (s, 3H), 1.01 (s, 3H). | LCMS (ES, m/z): 566 [M + H]+, Rt 0.518 min. | Column: (R,R)- WHELK-01- Kromasil, 5*25 cm, 5 um; Mobile Phase A: Hex(0.5% IPAmine)-- HPLC, Mobile Phase B: EtOH-- HPLC; Flow rate: 20 mL/min; Gradient: 50% B to 50% B in 12 min; Wave Length: 220/254 nm; RT1(min): 8.36; RT2(min): 10.39 |
| 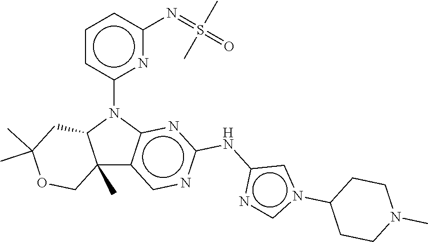 | | 1H NMR (400 MHz, DMSO-d6) δ 9.10 (s, 1H), 7.97-7.91 (m, 3H), 7.54 (t, J = 8.0 Hz, 1H), 7.46 (s, 1H), 6.28 (d, J = 8.0 Hz, 1H), 4.53- 4.49 (m, 1H), 4.06-4.00 (m, 1H), 3.82-3.79 (m, 1H), 3.64-3.61 (m, 1H), 3.48 (s, 3H), 3.29 (s, 3H), 2.88-2.82 (m, 2H), 2.45-2.40 (m, 1H), 2.20 (s, 3H), 2.06-1.87 (m, 6H), 1.53-1.47 (m, 1H), 1.25 (s, 3H), 1.22 (s, 3H), 1.01 (s, 3H). | LCMS (ES, m/z): 566 [M + H]+, Rt 0.520 min. | |
| 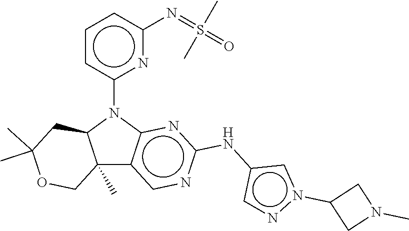 | 207, 208 | 1H NMR (300 MHz, DMSO-d6) δ 9.16 (s, 1H), 8.01-7.98 (m, 3H), 7.58-7.52 (m, 2H), 6.32 (d, J = 7.8 Hz, 1H), 4.87-4.79 (m, 1H), 4.51- 4.42 (m, 1H), 3.83-3.79 (m, 1H), 3.69-3.61 (m, 3H), 3.47 (s, 3H), 3.34-3.29 (m, 5H), 2.50-2.49 (m, 1H), 2.30 (s, 3H), 1.52-1.49 (m, 1H), 1.25 (s, 3H), 1.22 (s, 3H), 1.01 (s, 3H). | LCMS (ES, m/z): 538 [M + H]+, Rt 0.715 min. | Column: CHIRAL ART Cellulose-SB, 2*25 cm, 5 um; Mobile Phase A: Hex(0.5% IPAmine)-- HPLC, Mobile Phase B: EtOH-- HPLC; Flow rate: 20 mL/min; Gradient: 30% B to 30% B in 18 min; Wave Length: 220/254 nm; RT1(min): 12.23; RT2(min): 14.95 |
| 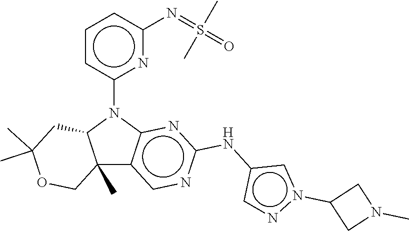 | | 1H NMR (300 MHz, DMSO-d6) δ 9.16 (s, 1H), 8.01-7.98 (m, 3H), 7.58-7.52 (m, 2H), 6.32 (d, J = 7.8 Hz, 1H), 4.87-4.79 (m, 1H), 4.51- 4.42 (m, 1H), 3.83-3.79 (m, 1H), 3.69-3.61 (m, 3H), 3.47 (s, 3H), 3.32-3.28 (m, 5H), 2.50-2.49 (m, 1H), 2.30 (s, 3H), 1.52-1.49 (m, 1H), 1.25 (s, 3H), 1.22 (s, 3H), 1.01 (s, 3H). | LCMS (ES, m/z): 538 [M + H]+, Rt 0.716 min. | |
| 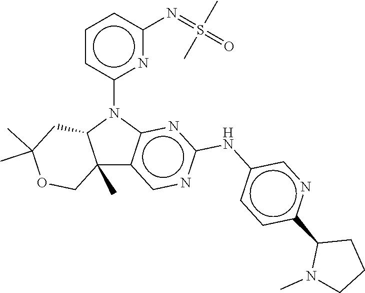 | 209, 210 | 1H NMR (300 MHz, DMSO-d6) δ 9.43 (s, 1H), 8.74 (d, J = 2.7 Hz, 1H), 8.20-8.17 (m, 1H), 8.08-8.05 (m, 2H), 7.58-7.52 (m, 1H), 7.35 (d, J = 8.4 Hz, 1H), 6.35 (d, J = 7.8 Hz, 1H), 4.56-4.55 (m, 1H), 3.87-3.83 (m, 1H), 3.69- 3.64 (m, 1H), 3.50 (s, 3H), 3.38 (s, 3H), 3.20- 3.14 (m, 2H), 2.49-2.42(m, 1H), 2.29-2.16(m, 1H), 2.14 (s, 4H), 1.91-1.72(m, 3H), 1.57-1.50 (m, 1H), 1.28 (s, 3H), 1.24 (s, 3H), 1.03 (s, 3H). | LCMS (ES, m/z): 563 [M + H]+, Rt 1.052 min. | Column: CHIRAL ART Cellulose-SC, 2 × 25 cm, 5 um; Mobile Phase A: Hex: DCM = 3:1 (0.5% 2 M NH3 × MeOH)-- HPLC, Mobile Phase B: EtOH-- HPLC(50% to 50% in 12 min); Flow rate: 20 mL/min; Wave Length: 220/254 nm; RT1(min): 5.54; RT2(min): 10.09 |
| 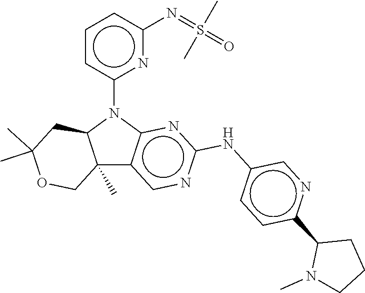 | | 1H NMR (300 MHz, DMSO-d6) δ 9.43 (s, 1H), 8.73 (d, J = 2.4 Hz, 1H), 8.21-8.17 (m, 1H), 8.08-8.05 (m, 2H), 7.55 (t, J = 7.8 Hz, 1H), 7.35 (d, J = 8.7 Hz, 1H), 6.35 (d, J = 7.8 Hz, 1H), 4.58-4.53 (m, 1H), 3.87-3.83 (m, 1H), 3.68-3.64 (m, 1H), 3.50 (s, 3H), 3.31 (s, 3H), 3.22-3.12 (m, 2H), 2.49-2.42 (m, 1H), 2.29- 2.23 (m, 1H), 2.19-2.13 (m, 4H), 1.82-1.67 (m, 3H), 1.57-1.49(m, 1H), 1.28 (s, 3H), 1.24 (s, 3H), 1.03 (s, 3H). | LCMS (ES, m/z): 563 [M + H]+, Rt 1.064 min. | |
| 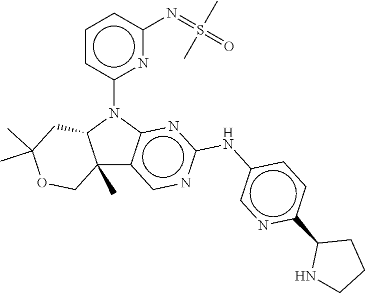 | 211, 212 | 1H NMR (300 MHz, DMSO-d6) δ 9.42 (s, 1H), 8.76 (s, 1H), 8.18-8.14 (m, 1H), 8.09-8.05 (m, 2H), 7.56 (t, J = 8.1 Hz, 1H), 7.37 (d, J = 8.4 Hz, 1H), 6.35 (d, J = 7.8 Hz, 1H), 4.58-4.53 (m, 1H), 4.11-4.07 (m, 1H), 3.87-3.83 (m, 1H), 3.69-3.65 (m, 1H), 3.50 (s, 3H), 3.35- 3.31 (m, 4H), 3.07-3.02 (m, 1H), 2.89-2.85 (m, 1H), 2.47-2.43 (m, 1H), 2.10-2.09 (m, 1H), 1.77-1.64 (m, 3H), 1.57-1.50 (m, 1H), 1.29 (s, 3H), 1.24 (s, 3H), 1.03 (s, 3H). | LCMS (ES, m/z): 549 [M + H]+. Rt 0.547 min. | Column: CHIRAL ART Cellulose-SC, 2 × 25 cm, 5 um; Mobile Phase A: Hex: DCM = 3:1 (0.5% 2 M NH3—MeOH)-- HPLC, Mobile Phase B: EtOH-- HPLC(50% to 50% in 12 min); Flow rate: 20 mL/min; Wave Length: 220/254 nm; RT1(min): 5.54; RT2(min): 10.09 |
| 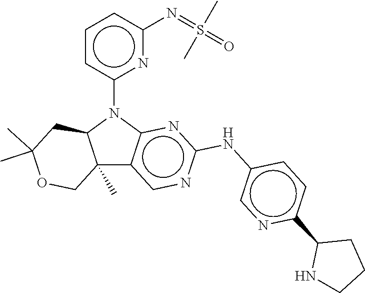 | | 1H NMR (300 MHz, DMSO-d6) δ 9.43 (s, 1H), 8.76 (s, 1H), 8.17-8.14 (m, 1H), 8.09-8.05 (m, 2H), 7.56 (t, J = 8.1 Hz, 1H), 7.37 (d, J = 8.4 Hz, 1H), 6.35 (d, J = 7.8 Hz, 1H), 4.58-4.53 (m, 1H), 4.12-4.07 (m, 1H), 3.87-3.83 (m, 1H), 3.69-3.65 (m, 1H), 3.50 (s, 3H), 3.35- 3.31 (m, 4H), 3.07-3.02 (m, 1H), 2.89-2.85 (m, 1H), 2.47-2.43 (m, 1H), 2.10-2.09 (m, 1H), 1.77-1.50 (m, 4H), 1.29 (s, 3H), 1.24 (s, 3H), 1.03 (s, 3H). | LCMS (ES, m/z): 549 [M + H]+. Rt 0.545 min. | |
| 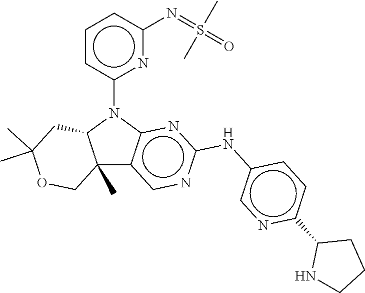 | 213, 214 | 1H NMR (300 MHz, DMSO-d6) δ 9.42 (s, 1H), 8.73 (s, 1H), 8.14-8.02 (m, 3H), 7.54 (t, J = 8.1 Hz, 1H), 7.35 (d, J = 8.4 Hz, 1H), 6.33 (d, J = 7.8 Hz, 1H), 4.56-4.51 (m, 1H), 4.05 (t, J = 7.2 Hz, 1H), 3.82-3.76 (m, 1H), 3.64-3.57 (m, 1H), 3.47-3.44 (m, 4H), 3.29 (s, 3H), 3.05- 3.01 (m, 1H), 2.90-2.73 (m, 1H), 2.57-2.40 (m, 1H), 2.13-2.01 (m, 1H), 1.76-1.48 (m, 4H), 1.26 (s, 3H), 1.22 (s, 3H), 1.00 (s, 3H). | LCMS (ES, m/z): 549 [M + H]+, Rt 0.541 min. | Column: CHIRAL ART Cellulose-SC, 2*25 cm, 5 um; Mobile Phase A: Hex: DCM = 3:1 (0.5% 2 M NH3—MeOH)-- HPLC, Mobile Phase B: EtOH-- HPLC; Flow rate: 20 mL/min; Gradient: 10% B to 10% B in 14 min; Wave Length: 220/254 nm; RT1(min): 10.06; RT2(min): 12.28; |
| 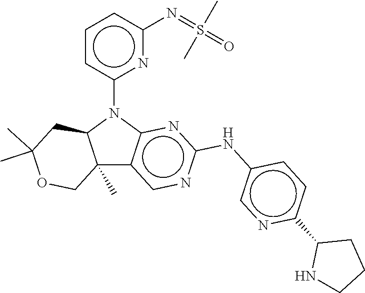 | | 1H NMR (300 MHz, DMSO-d6) δ 9.42 (s, 1H), 8.76 (s, 1H), 8.17-8.04 (m, 3H), 7.56 (t, J = 8.1 Hz, 1H), 7.37 (d, J = 8.4 Hz, 1H), 6.35 (d, J = 7.8 Hz, 1H), 4.58-4.53 (m, 1H), 4.07 (t, J = 7.2 Hz, 1H), 3.87-3.82 (m, 1H), 3.66-3.61 (m, 1H), 3.55-3.50 (m, 4H), 3.31 (s, 3H), 3.08- 3.01 (m, 1H), 2.90-2.81 (m, 1H), 2.47-2.43 (m, 1H), 2.12 -2.03 (m, 1H), 1.76-1.49 (m, 4H), 1.28 (s, 3H), 1.24 (s, 3H), 1.03 (s, 3H). | LCMS (ES, m/z): 549 [M + H]+, Rt 0.538 min. | |
| 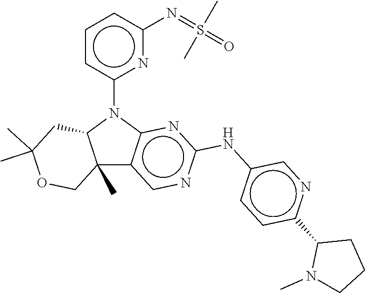 | 215, 216 | 1H NMR (300 MHz, DMSO-d6) δ 9.43 (s, 1H), 8.74 (s, 1H), 8.21-8.17 (m, 1H), 8.08-8.05 (m, 2H), 7.55 (t, J = 8.1 Hz, 1H), 7.35 (d, J = 8.4 Hz, 1H), 6.35 (d, J = 7.5 Hz, 1H), 4.58-4.53 (m, 1H), 3.87-3.82 (m, 1H), 3.66-3.61 (m, 1H), 3.50 (s, 3H), 3.31 (s, 3H), 3.22-3.12 (m, 2H), 2.51-2.43 (m, 1H), 2.30-2.24 (m, 1H), 2.18-2.12 (m, 4H), 1.82-1.68 (m, 3H), 1.57- 1.50 (m, 1H), 1.29 (s, 3H), 1.24 (s, 3H), 1.03 (s, 3H). | LCMS (ES, m/z): 563 [M + H]+, Rt 0.629 min. | Column: CHIRAL ART Cellulose-SB, 2 × 25 cm, 5 um; Mobile Phase A:Hex(0.5% 2 M NH3— MeOH)--HPLC, Mobile Phase B: EtOH--HPLC; Flow rate: 20 mL/min; Gradient: 5 B to 5 B in 31 min; 220/254 nm; RT1: 25.408; RT2: 28.039 |
| 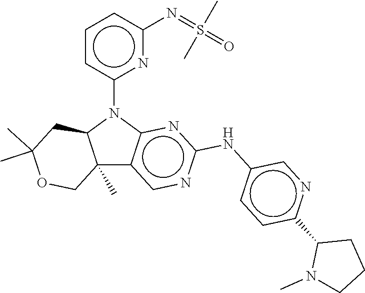 | | 1H NMR (300 MHz, DMSO-d6) δ 9.36 (s, 1H), 8.67 (s, 1H), 8.19-8.09 (m, 1H), 8.01-7.97 (m, 2H), 7.47 (t, J = 8.1 Hz, 1H), 7.28 (d, J = 8.4 Hz, 1H), 6.27 (d, J = 7.8 Hz, 1H), 4.50-4.45 (m, 1H), 3.78-3.72 (m, 1H), 3.66-3.61 (m, 1H), 3.42 (s, 3H), 3.23 (s, 3H), 3.18-3.05 (m, 2H), 2.43-2.35 (m, 1H), 2.31-3.25 (m, 1H), 2.15-2.07 (m, 4H), 1.86-1.62 (m, 3H), 1.49- 1.42 (m, 1H), 1.21 (s, 3H), 1.16 (s, 3H), 0.95 (s, 3H). | LCMS (ES, m/z): 563 [M + H]+, Rt 0.616 min. | |
| 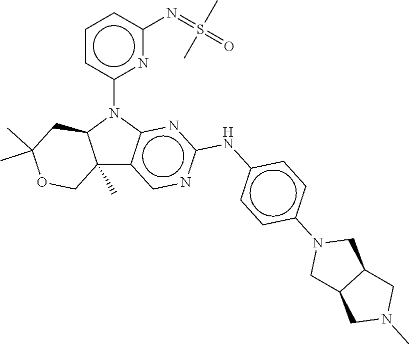 | 217, 218 | 1H NMR (400 MHz, DMSO-d6) δ 8.93 (s, 1H), 8.12 (d, J = 8.0 Hz, 1H), 7.94 (s, 1H), 7.54- 7.48 (m, 3H), 6.62 (d, J = 9.2 Hz, 2H), 6.31 (d, J = 7.6 Hz, 1H), 4.53-4.49 (m, 1H), 3.82-3.79 (m, 1H), 3.65-3.62 (m, 1H), 3.32 (s, 3H), 3.29-3.27 (m, 5H), 3.04-3.01 (m, 2H), 2.86- 2.84 (m, 2H), 2.60-2.56 (m, 2H), 2.47-2.42 (m, 1H), 2.37-2.34 (m, 2H), 2.22 (s, 3H), 1.52-1.46 (m, 1H), 1.25 (s, 3H), 1.22 (s, 3H), 1.01 (s, 3H). | LCMS (ES, m/z): 603 [M + H]+, Rt 0.568 min. | Column: CHIRAL ART Cellulose-SB, 2*25 cm, 5 um; Mobile Phase A: Hex(0.5% 2 M NH3—MeOH)-- HPLC, Mobile Phase B: EtOH-- HPLC; Flow rate: 20 mL/min; Gradient: 30% B to 30% B in 30 min; Wave Length: 220/254 nm; RT1(min): 17.88; RT2(min): 24.47 |
| 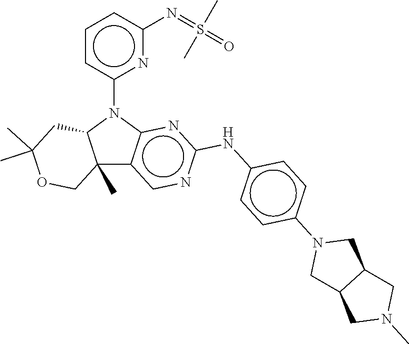 | | 1H NMR (400 MHz, DMSO-d6) δ 8.93 (s, 1H), 8.12 (d, J = 8.4 Hz, 1H), 7.94 (s, 1H), 7.54- 7.48 (m, 3H), 6.62 (d, J = 8.8 Hz, 2H), 6.31 (d, J = 7.6 Hz, 1H), 4.53-4.49 (m, 1H), 3.82-3.79 (m, 1H), 3.65-3.62 (m, 1H), 3.48 (s, 3H), 3.29-3.27 (m, 5H), 3.04-3.00 (m, 2H), 2.86- 2.84 (m, 2H), 2.60-2.55 (m, 2H), 2.47-2.42 (m, 1H), 2.37-2.34 (m, 2H), 2.21 (s, 3H), 1.52-1.46 (m, 1H), 1.25 (s, 3H), 1.22 (s, 3H), 1.01 (s, 3H). | LCMS (ES, m/z): 603 [M + H]+, Rt 0.574 min. | |
| 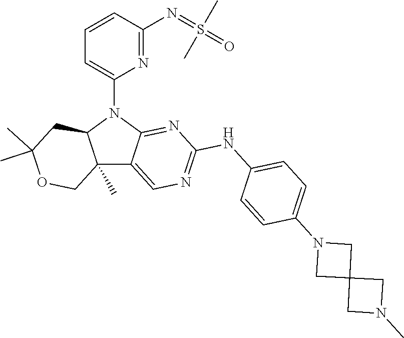 | 219, 220 | 1H NMR (300 MHz, DMSO-d6) δ 8.92 (s, 1H), 8.08 (d, J = 8.1 Hz, 1H), 7.93 (s, 1H), 7.53- 7.45 (m, 3H), 6.39-6.36 (m, 2H), 6.30 (d, J = 7.2 Hz, 1H), 4.53-4.48 (m, 1H), 3.82-3.79 (m, 5H), 3.64-3.58 (m, 1H), 3.53-3.44 (m, 4H), 3.31-3.28 (m, 6H), 2.49-2.40 (m, 1H), 2.22 (s, 3H), 1.52-1.44 (m, 1H), 1.25 (s, 3H), 1.22 (s, 3H), 1.01 (s, 3H). | LCMS (ES, m/z): 589 [M + H]+, Rt 0.570 min. | Column: CHIRAL ART Cellulose-SB, 2*25 cm, 5 um; Mobile Phase A: MtBE(0.5% 2 M NH3—MeOH)-- HPLC, Mobile Phase B: EtOH-- HPLC; Flow rate: 20 mL/min; Gradient: 15% B to 15% B in 12 min; Wave Length: 220/254 nm; RT1(min): 7.677; RT2(min): 9.54 |
| 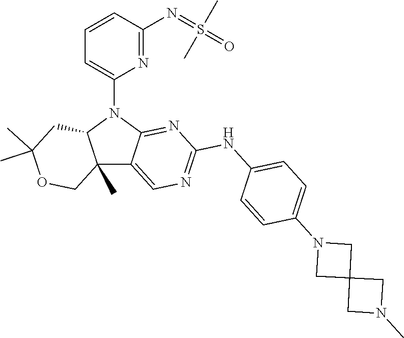 | | 1H NMR (300 MHz, DMSO-d6) δ 8.91 (s, 1H), 8.08 (d, J = 8.1 Hz, 1H), 7.93 (s, 1H), 7.53- 7.45 (m, 3H), 6.39-6.28 (m, 3H), 4.53-4.48 (m, 1H), 3.82-3.79 (m, 5H), 3.64-3.60 (m, 1H), 3.47 (s, 3H), 3.28 (s, 3H), 3.33-3.25 (m, 4H), 2.49-2.40 (m, 1H), 2.18 (s, 3H), 1.52- 1.45 (m, 1H), 1.25 (s, 3H), 1.22 (s, 3H), 1.01 (s, 3H). | LCMS (ES, m/z): 589 [M + H]+, Rt 0.547 min | |

### Examples 234 and 235: (S)-4-methyl-3-(6-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)pyridin-2-yl)oxazolidin-2-one and (S)-4-methyl-3-(6-((4bS,8aS)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)pyridin-2-yl)oxazolidin-2-one

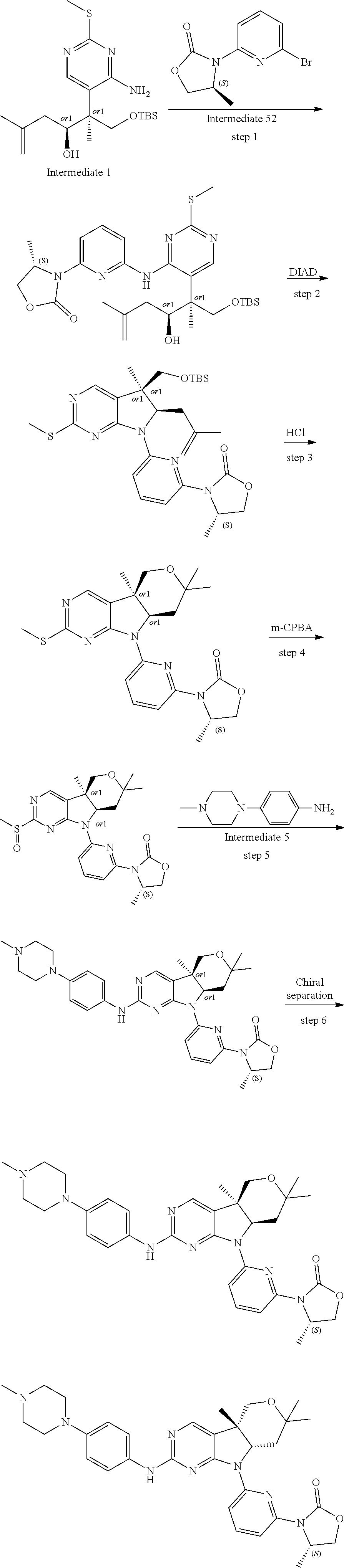

### Step 1. 3-(6-((5-rel-((2S,3S)-1-((tert-butyldimethylsilyl)oxy)-3-hydroxy-2,5-dimethylhex-5-en-2-yl)-2-(methylthio)pyrimidin-4-yl)amino)pyridin-2-yl)-(S)-4-methyloxazolidin-2-one

A solution of rel-(2S,3S)-2-(4-amino-2-(methylthio)pyrimidin-5-yl)-1-((tert-butyldimethylsilyl)oxy)-2,5-dimethylhex-5-en-3-ol (Intermediate 1, 1.1 g, 2.77 mmol, 1 eq.), (S)-3-(6-bromopyridin-2-yl)-4-methyloxazolidin-2-one (Intermediate 52, 853 mg, 3.32 mmol, 1.2 eq.), BINAP Pd G2 (258 mg, 276.63 umol, 0.1 eq.), BINAP (172 mg, 276.63 umol, 0.1 eq.) and Cs₂CO₃ (1.80 g, 5.53 mmol, 2 eq.) in dioxane (5 mL) was stirred for 2 h at 100° C. The reaction was monitored by LC-MS. The mixture was allowed to cool down to room temperature. The resulting mixture was diluted with brine (30 mL) and was extracted with ethyl acetate (3×30 mL). The combined organic layers were dried over anhydrous Na₂SO₄. After filtration, the filtrate was concentrated under reduced pressure. The residue was purified by silica gel column chromatography (eluting with 1:4 EA/PE) to afford 3-(6-((5-rel-((2S,3S)-1-((tert-butyldimethylsilyl)oxy)-3-hydroxy-2,5-dimethylhex-5-en-2-yl)-2-(methylthio)pyrimidin-4-yl)amino)pyridin-2-yl)-(S)-4-methyloxazolidin-2-one (1.3 g, 2.22 mmol, 81% yield) as a yellow solid. LCMS (ES, m/z): 574 [M+H]⁺, Rt 0.925 min.

### Step 2. 3-(6-rel-((5R,6R)-5-(((tert-butyldimethylsilyl)oxy)methyl)-5-methyl-6-(2-methylallyl)-2-(methylthio)-5,6-dihydro-7H-pyrrolo[2,3-d]pyrimidin-7-yl)pyridin-2-yl)-(S)-4-methyloxazolidin-2-one

To a solution of 3-(6-((5-rel-((2S,3S)-1-((tert-butyldimethylsilyl)oxy)-3-hydroxy-2,5-dimethylhex-5-en-2-yl)-2-(methylthio)pyrimidin-4-yl)amino)pyridin-2-yl)-(S)-4-methyloxazolidin-2-one (2.1 g, 3.66 mmol, 1 eq.) and PPh₃ (1.92 g, 7.32 mmol, 2 eq.) in THF (30 mL) was stirred for 5 min at 0° C. under nitrogen atmosphere. To above solution was added DIAD (1.48 g, 7.32 mmol, 2 eq.) dropwise at 0° C. The mixture was stirred for 3 h at 50° C. under N₂ atmosphere. The reaction was monitored by LC-MS. The resulting mixture was cooled to room temperature and was concentrated under reduced pressure. The residue was purified by silica gel column chromatography (eluting with 1:5 EA/PE) to afford 3-(6-rel-((5R,6R)-5-(((tert-butyldimethylsilyl)oxy)methyl)-5-methyl-6-(2-methylallyl)-2-(methylthio)-5,6-dihydro-7H-pyrrolo[2,3-d]pyrimidin-7-yl)pyridin-2-yl)-(S)-4-methyloxazolidin-2-one (1.8 g, 3.08 mmol, 88% yield) as a yellow solid. LCMS (ES, m/z): 556 [M+H]⁺, Rt 1.858 min.

### Step 3. (S)-4-methyl-3-(6-rel-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)oxazolidin-2-one

A solution of 3-(6-rel-((5R,6R)-5-(((tert-butyldimethylsilyl)oxy)methyl)-5-methyl-6-(2-methylallyl)-2-(methylthio)-5,6-dihydro-7H-pyrrolo[2,3-d]pyrimidin-7-yl)pyridin-2-yl)-(S)-4-methyloxazolidin-2-one (1.6 g, 2.88 mmol, 1 eq.) in dioxane (10 mL) was stirred for 5 min at 0° C. To above solution was added HCl (4 M, 16 mL, 20 eq.) dropwise at 0° C. The mixture was stirred for 3 h at room temperature. The reaction was monitored by LC-MS. The mixture was neutralized to pH=8 with saturated NaHCO₃. The resulting mixture was extracted with EA (3×50 mL). The combined organic layers were dried over anhydrous Na₂SO₄. After filtration, the filtrate was concentrated under reduced pressure. The residue was purified by silica gel column chromatography (eluting with 1:1 EA/PE) to afford (S)-4-methyl-3-(6-rel-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)oxazolidin-2-one (1.1 g, 2.37 mmol, 84% yield) as a white solid. LCMS (ES, m/z): 442 [M+H]⁺, Rt 0.733 min.

### Step 4. (S)-4-methyl-3-(6-rel-((4bR,8aR)-4b,7,7-trimethyl-2-(methylsulfinyl)-4b,7,8,8a-tetrahydropyrano[3′,4′:4,5]pyrrolo[2,3-d]pyrimidin-9(5H)-yl)pyridin-2-yl)oxazolidin-2-one

Into a solution of (S)-4-methyl-3-(6-rel-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)oxazolidin-2-one (2.0 g, 4.46 mmol, 1 eq.) in DCM (3 mL) was added m-CPBA (1.17 mg, 6.80 mmol, 1.5 eq.) in portions at 0° C. The mixture was stirred for 3 h at room temperature. The reaction was monitored by LC-MS. The mixture was concentrated under reduced pressure to afford (S)-4-methyl-3-(6-rel-((4bR,8aR)-4b,7,7-trimethyl-2-(methylsulfinyl)-4b,7,8,8a-tetrahydropyrano[3′,4′:4,5]pyrrolo[2,3-d]pyrimidin-9(5H)-yl)pyridin-2-yl)oxazolidin-2-one (2.0 g, crude) as a yellow oil. LCMS (ES, m/z): 458[M+H]⁺, Rt 0.675 min.

### Step 5. (S)-4-methyl-3-(6-rel-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)pyridin-2-yl)oxazolidin-2-one

A solution of (S)-4-methyl-3-(6-rel-((4bR,8aR)-4b,7,7-trimethyl-2-(methylsulfinyl)-4b,7,8,8a-tetrahydropyrano[3′,4′:4,5]pyrrolo[2,3-d]pyrimidin-9(5H)-yl)pyridin-2-yl)oxazolidin-2-one (2 g, 4.37 mmol, 1 eq.), 4-(4-methylpiperazin-1-yl)aniline (Intermediate 5, 1.00 g, 5.25 mmol, 1.2 eq.) and DIEA (2.82 g, 21.86 mmol, 5 eq.) in toluene (10 mL) was stirred for 12 h at 110° C. The reaction was monitored by LC-MS. The reaction was allowed to cool down to room temperature. The resulting mixture was concentrated under reduced pressure. The residue was purified by reverse phase chromatography (Column: C18; Mobile phase, A: water (containing 10 mmol/L NH₄HCO₃) and B: ACN (48% to 50% over 15 min); Detector, UV 254 nm, 280 nm) to afford (S)-4-methyl-3-(6-rel-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)pyridin-2-yl)oxazolidin-2-one (2 g, 3.42 mmol, 78% yield) as a yellow solid. LCMS (ES, m/z): 585 [M+H]⁺, Rt 0.692 min.

### Step 6. Separation of Enantiomers to Obtain (S)-4-methyl-3-(6-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)pyridin-2-yl)oxazolidin-2-one and (S)-4-methyl-3-(6-((4bS,8aS)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)pyridin-2-yl)oxazolidin-2-one (Examples 234 and 235)

The mixture obtained in Example 234/235—Step 5 (2.0 g, 3.42 mmol, 1 eq.) was separated by Chiral-HPLC (NB-Lux 5 um i-Cellulose-5, 2.12×25 cm, 5 μm; Mobile Phase A: CO₂, Mobile Phase B: IPA:ACN:DCM=2:2:1 (0.1% 2M NH₃-MEOH); Flow rate: 100 mL/min; Gradient: isocratic 50% B; Column Temperature(° C.): 35; Back Pressure(bar): 100; Wave Length: 220 nm; RT1(min): 7.63; RT2(min): 12.52) to afford Example 234 (816.4 mg, 1.38 mmol, 81% yield) as a yellow solid (the first eluting isomer) and Example 235 (842.6 mg, 1.43 mmol, 84% yield) as a yellow solid (the second eluting isomer).

Example 234: ¹H NMR (300 MHz, DMSO-d6) δ 9.12 (s, 1H), 8.42 (d, J=8.1 Hz, 1H), 8.01 (s, 1H), 7.83 (t, J=8.1 Hz, 1H), 7.66 (d, J=8.1 Hz, 1H), 7.55 (d, J=9.0 Hz, 2H), 6.91 (d, J=9.0 Hz, 2H), 4.85-4.75 (m, 1H), 4.63-4.53 (m, 1H), 4.53-4.44 (m, 1H), 4.15-4.05 (m, 1H), 3.85-3.77 (m, 1H), 3.72-3.65 (m, 1H), 3.12-3.03 (m, 4H), 2.50-2.44 (m, 4H), 2.40-2.30 (m, 1H), 2.24 (s, 3H), 1.66-1.52 (m, 1H), 1.49 (d, J=6.0 Hz, 3H), 1.29 (s, 3H), 1.26 (s, 3H), 1.08 (s, 3H). LCMS (ES, m/z): 585 [M+H]⁺, Rt 0.692 min.

Example 235: ¹H NMR (300 MHz, DMSO-d₆) δ 9.12 (s, 1H), 8.42 (d, J=8.1 Hz, 1H), 8.03 (s, 1H), 7.83 (d, J=8.1 Hz, 1H), 7.64 (d, J=7.8 Hz, 1H), 7.55 (d, J=8.7 Hz, 2H), 6.91 (d, J=9.0 Hz, 2H), 4.97-4.88 (m, 1H), 4.71-4.62 (m, 1H), 4.59-4.50 (m, 1H), 4.18-4.08 (m, 1H), 3.90-3.82 (m, 1H), 3.73-3.66 (m, 1H), 3.12-3.04 (m, 4H), 2.50-2.40 (m, 4H), 2.24 (s, 3H), 2.19-2.10 (m, 1H), 1.60-1.48 (m, 1H), 1.39 (d, J=6.3 Hz, 3H), 1.26 (s, 3H), 1.25 (s, 3H), 1.07 (s, 3H). LCMS (ES, m/z): 585 [M+H]⁺, Rt 0.675 min.

TABLE 15
The examples in the following table were synthesized according to the
appropriate method as demonstrated in Examples 234 and 235. Absolute stereochemistry of the
examples in the following table was arbitrarily assigned.

| | EX | | | Chiral |
|---|---|---|---|---|
| Structure | # | ¹H NMR | LCMS | conditions |
| 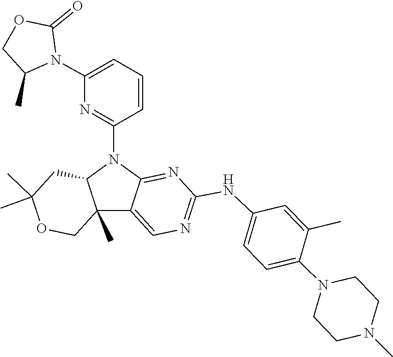 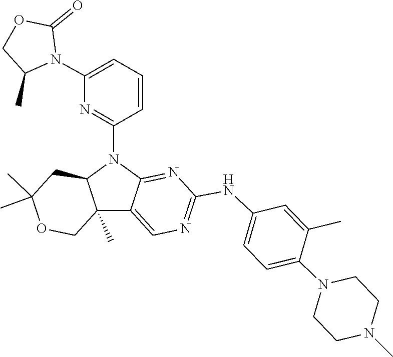 | 236, 237 | 1H NMR (300 MHz, DMSO-d6) δ 9.20 (s, 1H), 8.40 (d, J = 8.1 Hz, 1H), 8.03 (s, 1H), 7.81 (t, J = 8.1 Hz, 1H), 7.66 (d, J = 8.1 Hz, 1H), 7.65-7.53 (m, 1H), 7.40- 7.25 (m, 1H), 6.98 (d, J = 8.7 Hz, 1H), 4.95-4.72(m, 1H), 4.68-4.44 (m, 2H), 4.15-4.07 (m, 1H), 3.90-3.65 (m, 2H), 2.88-2.78 (m, 4H), 2.51-2.39 (m, 4H), 2.38-2.30 (m, 1H), 2.25 (s, 3H), 2.24 (s, 3H), 1.70-1.54 (m, 1H), 1.48 (d, J = 6.0 Hz, 3H), 1.28 (s, 3H), 1.24 (s, 3H), 1.08 (s, 3H). 1H NMR (300 MHz, DMSO-d6) δ 9.20 (s, 1H), 8.40 (d, J = 8.7 Hz, 1H), 8.05 (s, 1H), 7.82 (t, J = 8.1 Hz, 1H), 7.72-7.55 (m, 2H), 7.52-7.33 (m, 1H), 6.98 (d, J = 8.7 Hz, 1H), 5.07-4.82 (m, 1H), 4.75- 4.44 (m, 2H), 4.22-4.07 (m, 1H), 3.95- 3.65 (m, 2H), 2.88-2.78 (m, 4H), 2.49- 2.39(m, 4H), 2.25 (s, 3H), 2.24 (s, 3H), 2.18-2.13 (m, 1H), 1.65-1.45 (m, 1H), 1.48 (d, J = 6.0 Hz, 3H), 1.28 (s, 3H), 1.24 (s, 3H), 1.08 (s, 3H). | LCMS (ES, m/z): 599 [M + H]+. Rt 0.683 min. LCMS (ES, m/z): 599 [M + H]+. Rt 0.683 min. | Column: Reg- AD, 30*250 mm, 5 um; Mobile Phase A: Hex (0.5% 2M NH3-MeOH)- HPLC, Mobile Phase B: EtOH- HPLC; Flow rate: 38 mL/min; Gradient: 50 B to 50 B in 20 min; 220/254 nm; RT1: 11.2; RT2: 16.8 |
| 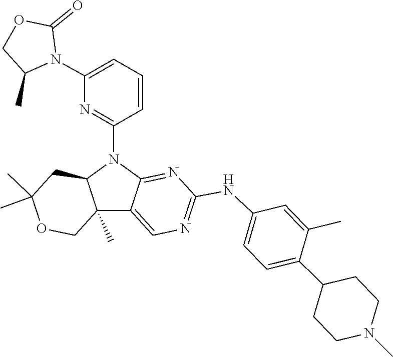 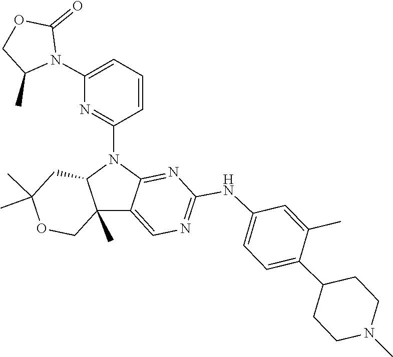 | 238, 239 | 1H NMR (300 MHz, DMSO-d6) δ 9.24 (s, 1H), 8.41 (d, J = 8.1 Hz, 1H), 8.06 (s, 1H), 7.82 (t, J = 8.1 Hz, 1H), 7.65 (d, J = 8.1 Hz, 1H), 7.63-7.55 (m, 1H), 7.50- 7.35 (m, 1H), 7.12 (d, J = 8.7 Hz, 1H), 5.01-4.85 (m, 1H), 4.75-4.65 (m, 1H), 4.55 (t, J = 8.1 Hz, 1H), 4.20-4.05 (m, 1H), 3.95-3.65 (m, 2H), 2.99-2.75 (m, 2H), 2.70-2.55 (m, 1H), 2.29 (s, 3H), 2.30-2.10 (m, 4H), 2.10-1.85 (m, 2H), 1.75-1.50 (m, 5H), 1.38 (d, J = 6.3 Hz, 3H), 1.27 (s, 3H), 1.25 (s, 3H), 1.06 (s, 3H). 1H NMR (300 MHz, DMSO-d6) δ 9.24 (s, 1H), 8.41 (d, J = 8.1 Hz, 1H), 8.06 (s, 1H), 7.82 (t, J = 8.1 Hz, 1H), 7.65 (d, J = 8.1 Hz, 1H), 7.63-7.55 (m, 1H), 7.50- 7.35 (m, 1H), 7.12 (d, J = 8.7 Hz, 1H), 4.90-4.75 (m, 1H), 4.65-4.45 (m, 2H), 4.18-4.05 (m, 1H), 3.89-3.78 (m, 1H), 3.75-3.68 (m, 1H), 2.99-2.85 (m, 2H), 2.72-2.58 (m, 1H), 2.40-2.30 (m, 1H), 2.29 (s, 3H), 2.21 (s, 3H), 2.08- 1.91 (m, 2H), 1.75-1.55 (m, 5H), 1.48 (d, J = 6.3 Hz, 3H), 1.27 (s, 3H), 1.25 (s, 3H), 1.08 (s, 3H). | LCMS (ES, m/z): 598 [M + H]+. Rt 0.683 min. LCMS (ES, m/z): 598 [M + H]+. Rt 0.683 min. | Column: Chiralpak IA, 2*25 cm, 5 um; Mobile Phase A: MTBE(0.5% 2M NH3- MeOH)-HPLC, Mobile Phase B: IPA-HPLC; Flow rate: 20 mL/min; Gradient: 20 B to 20 B in 17 min; 220/254 nm; RT1: 11.771; RT2: 13.319; |
| 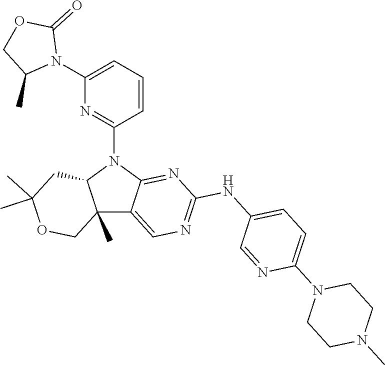 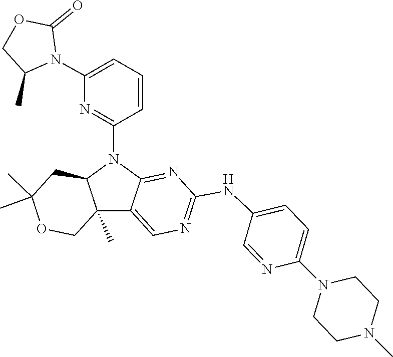 | 240, 241 | 1H NMR (400 MHz, DMSO-d6) δ 9.13 (s, 1H), 8.42-8.31 (m, 2H), 7.99 (s, 1H), 7.90 (d, J = 8.8 Hz, 1H), 7.79 (t, J = 8.4 Hz, 1H), 7.65 (d, J = 8.0 Hz, 1H), 6.84 (d, J = 9.6 Hz, 1H), 4.79-4.76 (m, 1H), 4.56 (t, J = 8.4 Hz, 1H), 4.49-4.45 (m, 1H), 4.11-4.08 (m, 1H), 3.81-3.78 (m, 1H), 3.68-3.65 (m, 1H), 3.41-3.38 (m, 4H), 2.49 (s, 3H), 2.36-2.31 (m, 1H), 2.29-2.21 (m, 4H), 1.60-1.54 (m, 1H), 1.47 (d, J = 6.8 Hz, 3H), 1.27 (s, 3H), 1.24 (s, 3H), 1.06 (s, 3H). 1H NMR (400 MHz, DMSO-d6) δ 9.13 (s, 1H), 8.41-8.35 (m, 2H), 8.00 (s, 1H), 7.91 (d, J = 8.8 Hz, 1H), 7.79 (t, J = 8.0 Hz, 1H), 7.63 (d, J = 8.0 Hz, 1H), 6.84 (d, J = 9.2 Hz, 1H), 4.93-4.90 (m, 1H), 4.67-4.64 (m, 1H), 4.53 (t, J = 8.4 Hz, 1H), 4.13-4.10 (m, 1H), 3.85-3.82 (m, 1H), 3.68-3.65 (m, 1H), 3.41-3.38 (m, 4H), 2.49 (s, 3H), 2.32-2.27 (m, 4H), 2.15-2.11 (m, 1H), 1.60-1.54 (m, 1H), 1.38 (d, J = 6.0 Hz, 3H), 1.27 (s, 3H), 1.24 (s, 3H), 1.05 (s, 3H). | LCMS (ES, m/z): 586 [M + H]+, Rt 0.648 min. LCMS (ES, m/z): 586 [M + H]+, Rt 0.650 min. | Column: CHIRALPAK IA, 2*25 cm, 5 μm; Mobile Phase A: MTBE(0.5% 2M NH3- MeOH)-HPLC, Mobile Phase B: EtOH-HPLC; Flow rate: 20 mL/min; Gradient: 50 B to 50 B in 10 min; 220/254 nm; RT1: 6.453; RT2: 8.348; |
| 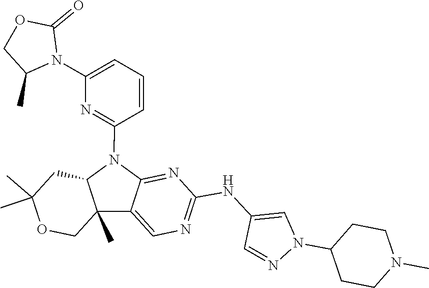 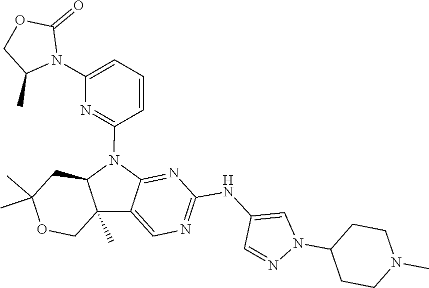 | 242, 243 | 1H NMR (400 MHz, DMSO-d6) δ 9.17 (s, 1H), 8.21 (br, 1H), 8.00 (s, 1H), 7.90 (s, 1H), 7.80 (t, J = 8.0 Hz, 1H), 7.66 (d, J = 8.0 Hz, 1H), 7.48 (s, 1H), 4.79-4.77 (m, 1H), 4.56 (t, J = 8.4 Hz, 1H), 4.48- 4.44 (m, 1H), 4.11-4.07 (m, 2H), 3.81- 3.78 (m, 1H), 3.68-3.65 (m, 1H), 2.91- 2.88 (m, 2H), 2.36-2.31 (m, 1H), 2.24 (s, 3H), 2.11-1.89 (m, 6H), 1.59-1.53 (m, 1H), 1.47 (d, J = 6.0 Hz, 3H), 1.27(s, 3H), 1.24 (s, 3H), 1.06 (s, 3H). 1H NMR (400 MHz, DMSO-d6) δ 9.17 (s, 1H), 8.32 (br, 1H), 8.01 (s, 1H), 7.90 (s, 1H), 7.80 (t, J = 8.0 Hz, 1H), 7.66 (d, J = 8.0 Hz, 1H), 7.48 (s, 1H), 4.97-4.80 (m, 1H), 4.70-4.60 (m, 1H), 4.54 (t, J = 8.4 Hz, 1H), 4.13-4.04 (m, 2H), 3.85- 3.81 (m, 1H), 3.68-3.62 (m, 1H), 2.87- 2.82 (m, 2H), 2.49 (s, 3H), 2.32-2.20 (m, 1H), 2.11-1.89 (m, 6H), 1.59-1.53 (m, 1H), 1.38 (d, J = 6.4 Hz, 3H), 1.24 (s, 3H), 1.23 (s, 3H), 1.05 (s, 3H). | LCMS (ES, m/z): 574 [M + H]+, Rt 0.558 min LCMS (ES, m/z): 574 [M + H]+, Rt 0.558 min. | Column: CHIRAL ART Cellulose-SC, 2*25 cm, 5 um; Mobile Phase A: Hex: DCM = 3:1 (0.5% 2M NH3- MeOH)-HPLC, Mobile Phase B: EtOH-HPLC; Flow rate: 20 mL/min; Gradient: 20 B to 20 B in 15 min; 220/254 nm; RT1: 9.784; RT2: 12.402 |
| 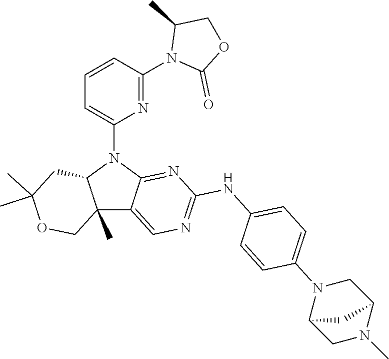 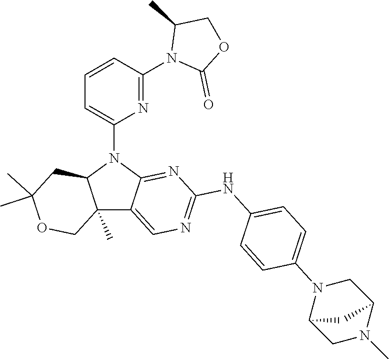 | 244, 245 | 1H NMR (300 MHz, DMSO-d6) δ 8.96 (s, 1H), 8.39 (d, J = 8.1 Hz, 1H), 7.95 (s, 1H), 7.76 (t, J = 8.1 Hz, 1H), 7.62 (d, J = 8.1 Hz, 1H), 7.42 (d, J = 8.4 Hz, 2H), 6.53 (d, J = 8.4 Hz, 2H), 4.79-4.73 (m, 1H), 4.54 (t, J = 8.1 Hz, 1H), 4.47-4.41 (m, 1H), 4.27-4.22 (m, 1H), 4.08 -4.01 (m, 1H), 3.82-3.61 (m, 3H), 3.14-3.05 (m, 2H), 2.81-2.73 (m, 2H), 2.39-2.22 (m, 4H), 1.90-1.71 (m, 2H), 1.61-1.48 (m, 1H), 1.45 (d, J = 6.0 Hz, 3H), 1.28 (s, 3H), 1.25 (s, 3H), 1.05 (s, 3H). 1H NMR (300 MHz, DMSO-d6) δ 8.96 (s, 1H), 8.39 (d, J = 8.1 Hz, 1H), 7.96 (s, 1H), 7.77 (t, J = 8.1 Hz, 1H), 7.60 (d, J = 8.1 Hz, 1H), 7.41 (d, J = 8.7 Hz, 2H), 6.57-6.48 (m, 2H), 4.93-4.87 (m, 1H), 4.68-4.62 (m, 1H), 4.52 (t, J = 8.1 Hz, 1H), 4.27-4.21 (m, 1H), 4.14-4.08 (m, 1H), 3.86-3.76 (m, 1H), 3.72-3.62 (m, 2H), 3.42-3.33 (m, 2H), 3.13-3.07 (m, 1H), 2.79-2.71 (m, 1H), 2.24 (s, 3H), 2.18-2.03 (m, 1H), 1.89-1.70 (m, 2H), 1.52-1.46 (m, 1H), 1.35 (d, J = 6.3 Hz, 3H), 1.25 (s, 6H), 1.04 (s, 3H). | LCMS (ES, m/z): 597 [M + H] +; RT: 0.687 min. LCMS (ES, m/z): 597 [M + H] +; RT: 0.685 min. | Column: CHIRALPAK IA, 2*25 cm, 5 μm; Mobile Phase A: MTBE (0.5% 2M NH3- MeOH)-HPLC, Mobile Phase B: EtOH-HPLC; Flow rate: 20 mL/min; Gradient: 50 B to 50 B in 10 min; 220/254 nm; RT1: 6.453; RT2: 8.348; |
| 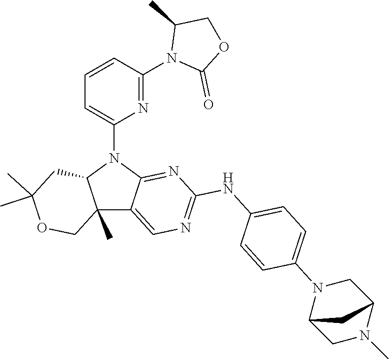 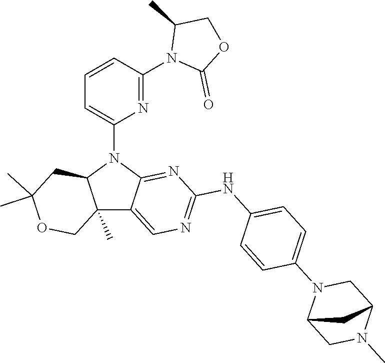 | 246, 247 | 1H NMR (400 MHz, DMSO-d6) δ 8.97 (s, 1H), 8.40 (d, J = 8.8 Hz, 1H), 7.96 (s, 1H), 7.78 (t, J = 8.4 Hz, 1H), 7.63 (d, J = 8.0 Hz, 1H), 7.43 (d, J = 8.8 Hz, 2H), 6.56-6.54 (m, 2H), 4.78-4.76 (m, 1H), 4.56 (t, J = 8.4 Hz, 1H), 4.48-4.44 (m, 1H), 4.26 (s, 1H), 4.11-4.08 (m, 1H), 3.80-3.77 (m, 1H), 3.67-3.64 (m, 1H), 3.48-3.35 (m, 1H), 3.18-3.13 (m, 1H), 2.84-2.77 (m, 1H), 2.71-2.55 (m, 2H), 2.35-2.30 (m, 4H), 1.88-1.85 (m, 1H), 1.80-1.78 (m, 1H), 1.59-1.53 (m, 1H), 1.47 (d, J = 6.4 Hz, 3H), 1.27 (s, 3H), 1.24 (s, 3H), 1.06 (s, 3H). 1H NMR (400 MHz, DMSO-d6) δ 8.97 (s, 1H), 8.40 (d, J = 4.4 Hz, 1H), 7.98 (s, 1H), 7.78 (t, J = 8.0 Hz, 1H), 7.62 (d, J = 8.0 Hz, 1H), 7.43 (d, J = 8.8 Hz, 2H), 6.55-6.53 (m, 2H), 4.92-4.90 (m, 1H), 4.66-4.62 (m, 1H), 4.55-4.51 (m, 1H), 4.27-4.21 (m, 1H), 4.12-4.09 (m, 1H), 3.84-3.81 (m, 1H), 3.68-3.65 (m, 1H), 3.42-3.33 (m, 1H), 3.15-3.11 (m, 1H), 2.81-2.76 (m, 1H), 2.54-2.51 (m, 2H), 2.26 (s, 3H), 2.15-2.11 (m, 1H), 1.87- 1.85 (m, 1H), 1.77-1.75 (m, 1H), 1.55- 1.51 (m, 1H), 1.37 (d, J = 6.0 Hz, 3H), 1.26 (s, 3H), 1.23 (s, 3H), 1.05 (s, 3H). | LCMS (ES, m/z): 597 [M + H]+, Rt 0.686 min LCMS (ES, m/z): 597 [M + H]+, Rt 0.688 min. | Column: CHIRALPAK IA, 2*25 cm, 5 um; Mobile Phase A: Hex: DCM = 3:1 (0.5% 2M NH3- MeOH)-HPLC, Mobile Phase B: EtOH-HPLC; Flow rate: 18 mL/min; Gradient: 50 B to 50 B in 23 min; 220/254 nm; RT1: 9.005; RT2: 16.704; Injection Volumn: 1.2 ml; Number Of Runs: 3; |
| 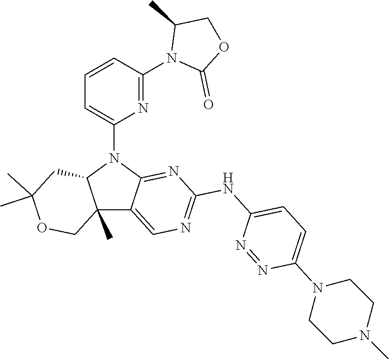 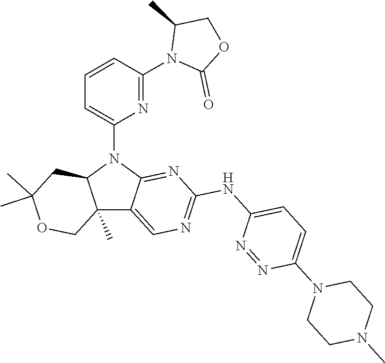 | 248, 249 | 1H NMR (300 MHz, DMSO-d6) δ 9.81 (s, 1H), 8.45 (d, J = 8.4 Hz, 1H), 8.15 (d, J = 9.6 Hz, 1H), 8.03 (s, 1H), 7.80 (t, J = 8.1 Hz, 1H), 7.65 (d, J = 8.1 Hz, 1H), 7.36 (d, J = 9.6 Hz, 1H), 4.85-4.78 (m, 1H), 4.58- 4.47 (m, 2H), 4.11-4.07 (m, 1H), 3.83-3.78 (m, 1H), 3.69-3.65 (m, 1H), 3.50-3.47 (m, 4H), 2.49-2.42 (m, 4H), 2.37-2.22 (m, 4H), 1.67-1.54 (m, 1H), 1.47 (d, J = 6.3 Hz, 3H), 1.28 (s, 3H), 1.26 (s, 3H), 1.06 (s, 3H). 1H NMR (300 MHz, DMSO-d6) δ 9.81 (s, 1H), 8.45 (d, J = 8.4 Hz, 1H), 8.15 (d, J = 9.6 Hz, 1H), 8.05 (s, 1H), 7.81 (t, J = 8.1 Hz, 1H), 7.64 (d, J = 7.8 Hz, 1H), 7.36 (d, J = 9.9 Hz, 1H), 4.94-4.90 (m, 1H), 4.70-4.65 (m, 1H), 4.53-4.50 (m, 1H), 4.13-4.09 (m, 1H), 3.87-3.83 (m, 1H), 3.73-3.66 (m, 1H), 3.50-3.47 (m, 4H), 2.49-2.39 (m, 4H), 2.27-2.12 (m, 4H), 1.67-1.54 (m, 1H), 1.47 (d, J = 6.3 Hz, 3H), 1.28 (s, 3H), 1.26 (s, 3H), 1.06 (s, 3H). | LCMS (ES, m/z): 587 [M + H]+, Rt 0.664 min. LCMS (ES, m/z): 587 [M + H]+, Rt 0.664 min | Column: CHIRALPAK ID, 2*25 cm, 5 um; Mobile Phase A: Hex: DCM = 3:1 (0.5% 2M NH3- MeOH)-HPLC, Mobile Phase B: EtOH-HPLC; Flow rate: 18 mL/min; Gradient: 50 B to 50 B in 29 min; 254/220 nm; RT1: 19.518; RT2: 25.182; |
| 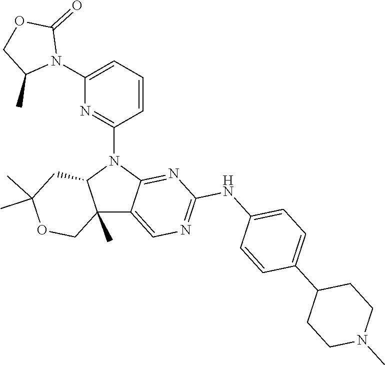 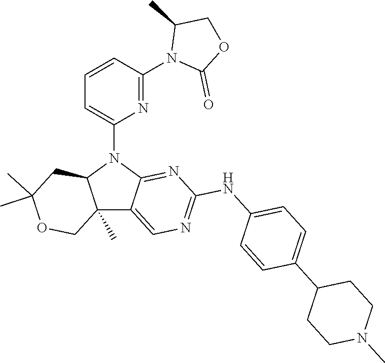 | 250, 251 | 1H NMR (300 MHz, DMSO-d6) δ 9.29 (s, 1H), 8.44 (d, J = 8.1 Hz, 1H), 8.04 (s, 1H), 7.86-7.80 (m, 1H), 7.69-7.61 (m, 3H), 7.18 (d, J = 8.7 Hz, 2H), 4.80-4.77 (m, 1H), 4.58-4.48 (m, 2H), 4.13-4.07 (m, 1H), 3.84-3.80 (m, 1H), 3.71-3.67 (m, 1H), 2.89-2.85 (m, 2H), 2.44-2.28 (m, 2H), 2.20 (s, 3H), 2.00-1.92 (m, 2H), 1.76-1.56 (m, 5H), 1.48 (d, J = 6.3 Hz, 3H), 1.30 (s, 3H), 1.26 (s, 3H), 1.08 (s, 3H). 1H NMR (400 MHz, DMSO-d6) δ 9.29 (s, 1H), 8.43 (d, J = 8.0 Hz, 1H), 8.04 (s, 1H), 7.82 (t, J = 8.4 Hz, 1H), 7.71-7.62 (m, 3H), 7.17 (d, J = 8.8 Hz, 2H), 4.83- 4.77 (m, 1H), 4.59-4.55 (m, 1H), 4.50- 4.47 (m, 1H), 4.11-4.08 (m, 1H), 3.83- 3.80 (m, 1H), 3.71-3.67 (m, 1H), 2.87- 2.85 (m, 2H), 2.39-2.32 (m, 2H), 2.19 (s, 3H), 1.98-1.92 (m, 2H), 1.71-1.60 (m, 5H), 1.47 (d, J = 6.4 Hz, 3H), 1.29 (s, 3H), 1.25 (s, 3H), 1.07 (s, 3H). | LCMS (ES, m/z): 584 [M + H]+. Rt 3.077 min. LCMS (ES, m/z): 584 [M + H]+. Rt 0.701 min. | Column: CHIRALPAK AS-H, 2*25 cm, 5 um; Mobile Phase A: Hex (0.5% 2M NH3-MeOH)- HPLC, Mobile Phase B: EtOH- HPLC; Flow rate: 20 mL/min; Gradient: 30 B to 30 B in 20 min; 220/254 nm; RT1: 12.526; RT2: 17.106 |
| 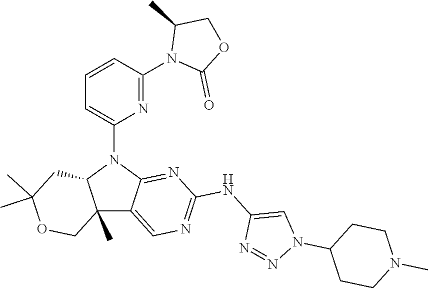 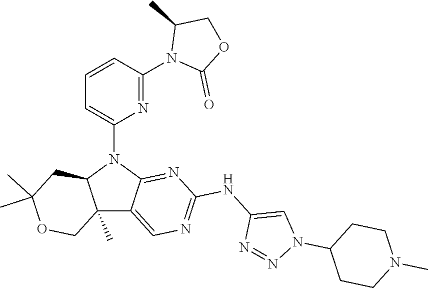 | 252, 253 | 1H NMR (300 MHz, DMSO-d6) δ 9.95 (s, 1H), 8.43 (br, 1H), 8.15 (s, 1H), 8.06 (s, 1H), 7.81 (t, J = 8.1 Hz, 1H), 7.68 (d, J = 8.1 Hz, 1H), 4.83-4.77 (m, 1H), 4.61-4.45 (m, 3H), 4.13-4.09 (m, 1H), 3.84-3.80 (m, 1H), 3.72-3.68 (m, 1H), 3.05-2.96 (m, 2H), 2.30 (s, 3H), 2.23- 2.01 (m, 7H), 1.63-1.56 (m, 1H), 1.49 (d, J = 6.3 Hz, 3H), 1.30 (s, 3H), 1.26 (s, 3H), 1.08 (s, 3H). 1H NMR (300 MHz, DMSO-d6) δ 9.95 (s, 1H), 8.42 (br, 1H), 8.15 (s, 1H), 8.08 (s, 1H), 7.81 (t, J = 8.1 Hz, 1H), 7.67 (d, J = 8.1 Hz, 1H), 4.96-4.92 (m, 1H), 4.71-4.66 (m, 1H), 4.58-4.44 (m, 2H), 4.15-4.11 (m, 1H), 3.89-3.84 (m, 1H), 3.72-3.68 (m, 1H), 3.01-2.95 (m, 2H), 2.29 (s, 3H), 2.19-2.02 (m, 7H), 1.59- 1.51 (m, 1H), 1.39 (d, J = 6.3 Hz, 3H), 1.27 (s, 3H), 1.25 (s, 3H), 1.07 (s, 3H). | LCMS (ES, m/z): 575 [M + H]+, Rt 2.823 min. LCMS (ES, m/z): 575 [M + H]+ , Rt 2.807 min. | Column: CHIRALPAK IA, 3 × 25 cm, 5 μm; Mobile Phase A: MtBE(0.5% 2M NH3- MeOH)-HPLC, Mobile Phase B: EtOH- HPLC(15% to 15% in 11 min); Flow rate: 15 mL/min; 220/254 nm; RT1: 7.77; RT2: 9.31 |
| 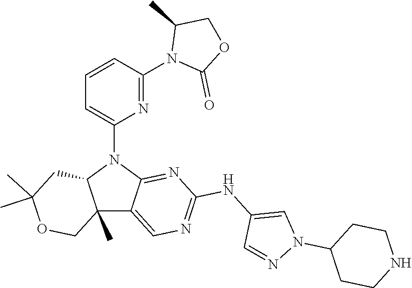 [SMILES: C[C@H]1COC(=O)N1c1cccc(N2c3nc(Nc4cnn(C5CCNCC5)c4)ncc3[C@]3(C)COC(C)(C)C[C@H]23)n1] 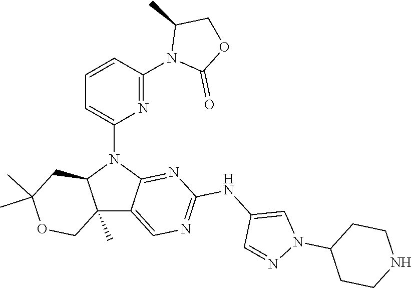 [SMILES: C[C@H]1COC(=O)N1c1cccc(N2c3nc(Nc4cnn(C5CCNCC5)c4)ncc3[C@@]3(C)COC(C)(C)C[C@@H]23)n1] | 254, 255 | 1H NMR (300 MHz, DMSO-d6) δ 9.21 (s, 1H), 8.37 (br, 1H), 8.02 (s, 1H), 7.85 (s, 1H), 7.82 (t, J = 8.1 Hz, 1H), 7.67 (d, J = 8.1 Hz, 1H), 7.47 (s, 1H), 4.83-4.73 (m, 1H), 4.57 (t, J = 8.1 Hz, 1H), 4.49- 4.42 (m, 1H), 4.22-4.07 (m, 2H), 3.86- 3.76 (m, 1H), 3.68-3.62 (m, 1H), 3.12- 3.05 (m, 2H), 2.68-2.55 (m, 2H), 2.41- 2.34 (m, 1H), 1.97-1.89 (m, 2H), 1.79- 1.67 (m, 2H), 1.62-1.51 (m, 1H), 1.48 (d, J = 6.0 Hz, 3H), 1.27 (s, 3H), 1.25 (s, 3H), 1.08 (s, 3H). 1H NMR (300 MHz, DMSO-d6) δ 9.21 (s, 1H), 8.37 (br, 1H), 8.03 (s, 1H), 7.86 (s, 1H), 7.80 (t, J = 8.1 Hz, 1H), 7.66 (d, J = 8.1 Hz, 1H), 7.47 (s, 1H), 4.99-4.85 (m, 1H), 4.71-4.62 (m, 1H), 4.54 (t, J = 8.1 Hz, 1H), 4.27-4.15 (m, 2H), 3.89- 3.79 (m, 1H), 3.73-3.63 (m, 1H), 3.09- 2.94 (m, 2H), 2.64-2.55 (m, 2H), 2.19- 2.11 (m, 1H), 1.98-1.87 (m, 2H), 1.78- 1.63 (m, 2H), 1.58-1.49 (m, 1H), 1.39 (d, J = 6.3 Hz, 3H), 1.25 (s, 6H), 1.06 (s, 3H). | LCMS (ES, m/z): 560 [M + H] +; RT: 0.649 min. LCMS (ES, m/z): 560 [M + H] +; RT: 0.647 min. | Column: CHIRALPAK IG, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 50% B to 50% B in 19 min; Wave Length: 254/220 nm; RT1(min): 9.28; RT2(min): 14.54; |
| 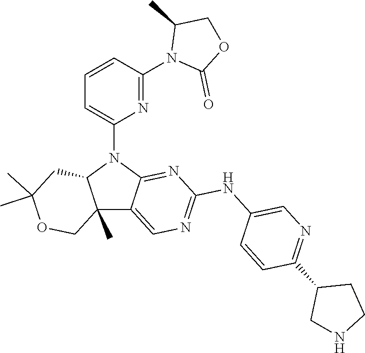 [SMILES: C[C@H]1COC(=O)N1c1cccc(N2c3nc(Nc4ccc([C@@H]5CCNC5)nc4)ncc3[C@]3(C)COC(C)(C)C[C@H]23)n1] 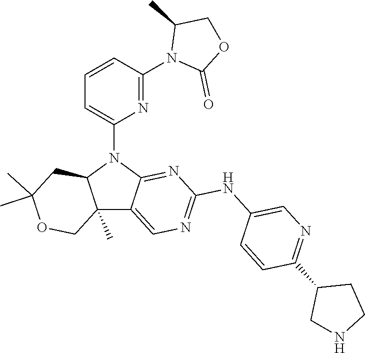 [SMILES: C[C@H]1COC(=O)N1c1cccc(N2c3nc(Nc4ccc([C@@H]5CCNC5)nc4)ncc3[C@@]3(C)COC(C)(C)C[C@@H]23)n1] | 256, 257 | 1H NMR (300 MHz, DMSO-d6) δ 9.48 (s, 1H), 8.75 (s, 1H), 8.39 (d, J = 8.1 Hz, 1H), 8.21-8.05 (m, 2H), 7.83 (t, J = 8.1 Hz, 1H), 7.69 (d, J = 8.1 Hz, 1H), 7.22 (d, J = 8.4 Hz, 1H), 4.85-4.75 (m, 1H), 4.63-4.45 (m, 2H), 4.15-4.10 (m, 1H), 3.84-3.65 (m, 2H), 3.30-3.22 (m, 1H), 3.20-3.10 (m, 1H), 3.02-2.70 (m, 3H), 2.40-2.25 (m, 1H), 2.13-2.02 (m, 1H), 1.95-1.80 (m, 1H), 1.68-1.54 (m, 1H), 1.49 (d, J = 6.0 Hz, 3H), 1.30 (s, 3H), 1.26 (s, 3H), 1.08 (s, 3H). min. 1H NMR (300 MHz, DMSO-d6) δ 9.48 (s, 1H), 8.75 (s, 1H), 8.39 (d, J = 8.1 Hz, 1H), 8.21-8.05 (m, 2H), 7.84 (t, J = 8.1 Hz, 1H), 7.67 (d, J = 8.1 Hz, 1H), 7.22 (d, J = 8.7 Hz, 1H), 4.99-4.89 (m, 1H), 4.74-4.65 (m, 1H), 4.60-4.50 (m, 1H), 4.18-4.09 (m, 1H), 3.92-3.65 (m, 2H), 3.30-3.10 (m, 3H), 3.02-2.70 (m, 2H), 2.20-2.00 (m, 2H), 1.95-1.80 (m, 1H), 1.63-1.50 (m, 1H), 1.39 (d, J = 6.0 Hz, 3H), 1.27 (s, 3H), 1.25 (s, 3H), 1.07 (s, 3H). | LCMS (ES, m/z): 557 [M + H]+, Rt 0.633 LCMS (ES, m/z): 557 [M + H]+, Rt 0.633 min | Column: CHIRALPAK IE, 2 × 25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 30% B to 30% B in 20 min; Wave Length: 254/220 nm; RT1(min): 14.09; RT2(min): 17.16; Sample Solvent: EtOH- HPLC; |
| 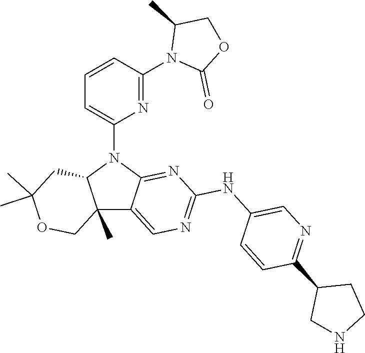 [SMILES: C[C@H]1COC(=O)N1c1cccc(N2c3nc(Nc4ccc([C@H]5CCNC5)nc4)ncc3[C@]3(C)COC(C)(C)C[C@H]23)n1] 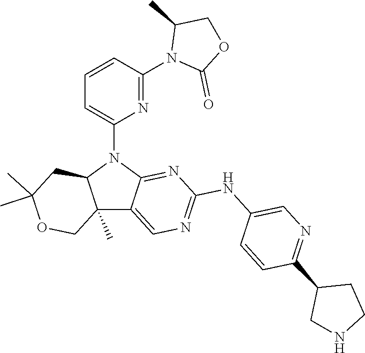 [SMILES: C[C@H]1COC(=O)N1c1cccc(N2c3nc(Nc4ccc([C@H]5CCNC5)nc4)ncc3[C@@]3(C)COC(C)(C)C[C@@H]23)n1] | 258, 259 | 1H NMR (400 MHz, DMSO-d6) δ 9.69- 9.35 (m, 1H), 8.85-8.65 (m, 1H), 8.38 (d, J = 8.0 Hz, 1H), 8.24-7.92(m, 2H), 7.83 (t, J = 8.4 Hz, 1H), 7.67 (d, J = 8.0 Hz, 1H), 7.35-7.15 (m, 1H), 4.95-4.65 (m, 1H), 4.57 (t, J = 8.0 Hz, 1H), 4.52- 4.39 (m, 1H), 4.15-4.05 (m, 1H), 3.86- 3.79 (m, 1H), 3.75-3.65 (m, 2H), 3.21- 3.12 (m, 1H), 3.02-2.85 (m, 3H), 2.38- 2.32 (m, 1H), 2.25-2.00 (m, 2H), 1.70- 1.55 (m, 1H), 1.47 (d, J = 6.8 Hz, 3H), 1.29 (s, 3H), 1.25 (s, 3H), 1.07 (s, 3H). 1H NMR (400 MHz, DMSO-d6) δ 9.62- 9.30 (m, 1H), 8.85-8.65 (m, 1H), 8.50- 8.38 (m, 1H), 8.22-7.95 (m, 2H), 7.83 (t, J = 8.1 Hz, 1H), 7.66 (d, J = 8.1 Hz, 1H), 7.30-7.08 (m, 1H), 5.05-4.85 (m, 1H), 4.85-4.65 (m, 1H), 4.54 (t, J = 8.4 Hz, 1H), 4.15-4.05 (m, 1H), 3.96-3.84 (m, 1H), 3.75-3.55 (m, 2H), 3.25-3.15 (m, 2H), 3.06-2.86 (m, 2H), 2.25-2.00 (m, 2H), 1.95-1.82 (m, 1H), 1.62-1.48 (m, 1H), 1.38 (d, J = 6.0 Hz, 3H), 1.26 (s, 3H), 1.24 (s, 3H), 1.06 (s, 3H). | LCMS (ES, m/z): 557 [M + H]+. Rt 0.633 min. LCMS (ES, m/z): 557 [M + H]+. Rt 0.633 min. | Column: XBridge Prep OBD C18 Column, 30*150 mm, 5 μm; Mobile Phase A: Water(10 mmol/L NH4HCO3 + 0.1% NH3.H2O), Mobile Phase B: ACN; Flow rate: 60 mL/min; Gradient: 34% B to 64% B in 7 min, 64% B; Wave Length: 254 nm; RT1(min): 5.3; Number Of Runs: 0 |
| 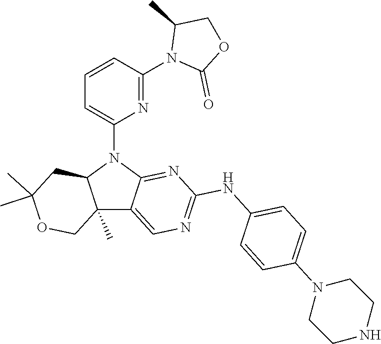 [SMILES: C[C@H]1COC(=O)N1c1cccc(N2c3nc(Nc4ccc(N5CCNCC5)cc4)ncc3[C@@]3(C)COC(C)(C)C[C@@H]23)n1] 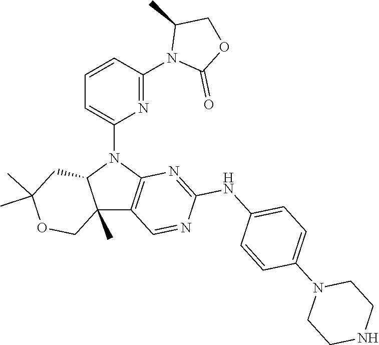 [SMILES: C[C@H]1COC(=O)N1c1cccc(N2c3nc(Nc4ccc(N5CCNCC5)cc4)ncc3[C@]3(C)COC(C)(C)C[C@H]23)n1] | 260, 261 | 1H NMR (300 MHz, DMSO-d6) δ 9.16 (s, 1H), 8.42 (d, J = 8.1 Hz, 1H), 8.01 (s, 1H), 7.82 (t, J = 8.1 Hz, 1H), 7.66 (d, J = 8.1 Hz, 1H), 7.56 (d, J = 8.7 Hz, 2H), 6.97-6.86 (m, 2H), 4.85-4.71 (m, 1H), 4.57 (t, J = 8.1 Hz, 1H), 4.50-4.42 (m, 1H), 4.13-4.05 (m, 1H), 3.86-3.76 (m, 1H), 3.73-3.63 (m, 1H), 3.10-3.03 (m, 4H), 3.02-2.87 (m, 4H), 2.44-2.25 (m, 1H), 1.64-1.51 (m, 1H), 1.48 (d, J = 6.0 Hz, 3H), 1.29 (s, 3H), 1.25 (s, 3H), 1.08 (s, 3H). 1H NMR (300 MHz, DMSO-d6) δ 9.17 (s, 1H), 8.41 (d, J = 8.1 Hz, 1H), 8.03 (s, 1H), 7.83 (t, J = 8.1 Hz, 1H), 7.68-7.53 (m, 3H), 6.97-6.86 (m, 2H), 4.99-4.86 (m, 1H), 4.69-4.62 (m, 1H), 4.55 (t, J = 8.1 Hz, 1H), 4.16-4.09 (m, 1H), 3.90- 3.80 (m, 1H), 3.73-3.63 (m, 1H), 3.17- 2.93 (m, 8H), 2.18-2.11 (m, 1H), 1.61- 1.52 (m, 1H), 1.38 (d, J = 6.3 Hz, 3H), 1.27 (s, 3H), 1.25 (s, 3H), 1.06 (s, 3H). | LCMS (ES, m/z): 571 [M + H] +; RT: 0.678 min. LCMS (ES, m/z): 571 [M + H] +; RT: 0.674 min. | Column: CHIRALPAK IG, 2*25 cm, 5 μm; Mobile Phase A: Hex: DCM = 2:1 (0.5% 2M NH3-MeOH)- HPLC, Mobile Phase B: EtOH- HPLC; Flow rate: 20 mL/min; Gradient: 30% B to 30% B in 25 min; Wave Length: 220/254 nm; RT1(min): 12.223; RT2(min): 18.472; Sample Solvent: EtOH- HPLC; Injection Volume: 0.8 mL; Number Of Runs: 5 |
| 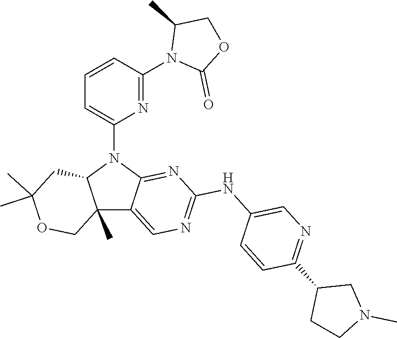 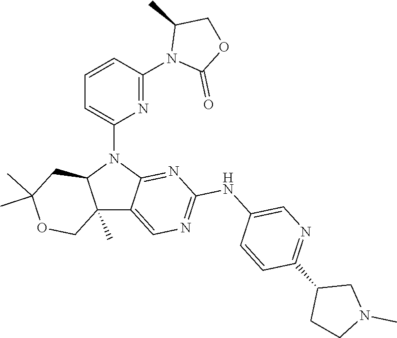 | 262, 263 | 1H NMR (300 MHz, DMSO-d6) δ 9.48 (s, 1H), 8.74 (d, J = 2.4 Hz, 1H), 8.38 (d, J = 8.1 Hz, 1H), 8.20-8.03 (m, 2H), 7.82 (t, J = 8.1 Hz, 1H), 7.67 (d, J = 8.1 Hz, 1H), 7.23 (d, J = 8.4 Hz, 1H), 4.88- 4.65 (m, 1H), 4.65-4.45 (m, 2H), 4.20- 4.01 (m, 1H), 4.01-2.59 (m, 2H), 3.43- 3.38 (m, 1H), 2.99-2.85 (m, 1H), 2.75- 2.64 (m, 2H), 2.28-2.24 (m, 5H), 2.10- 1.88 (m, 2H), 1.65-1.55 (m, 1H), 1.47 (d, J = 6.0 Hz, 3H), 1.29 (s, 3H), 1.25 (s, 3H), 1.07 (s, 3H). 1H NMR (300 MHz, DMSO-d6) δ 9.48 (s, 1H), 8.74 (d, J = 2.4 Hz, 1H), 8.38 (d, J = 8.1 Hz, 1H), 8.20-8.03 (m, 2H), 7.82 (t, J = 8.1 Hz, 1H), 7.66 (d, J = 8.1 Hz, 1H), 7.23 (d, J = 8.4 Hz, 1H), 5.00- 4.80 (m, 1H), 4.75-4.60 (m, 1H), 4.67 (t, J = 4.5 Hz, 1H), 4.20-4.09 (m, 1H), 3.94-3.71 (m, 1H), 3.78-3.64 (m, 1H), 3.52-3.40 (m, 1H), 2.99-2.85 (m, 1H), 2.75-2.64 (m, 2H), 2.28-2.24 (m, 4H), 2.20-2.08 (m, 2H), 2.08-1.82 (m, 1H), 1.65-1,45 (m, 1H), 1.38 (d, J = 6.3 Hz, 3H), 1.26 (s, 3H), 1.24 (s, 3H), 1.06 (s, 3H). | LCMS (ES, m/z): 571 [M + H]+. Rt 0.658 min. LCMS (ES, m/z): 571 [M + H]+. Rt 0.642 min. | Column: CHIRALPAK IG, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 20% B to 20% B in 22 min; Wave Length: 220/254 nm; RT1(min): 15.14; RT2(min): 20.05; |
| 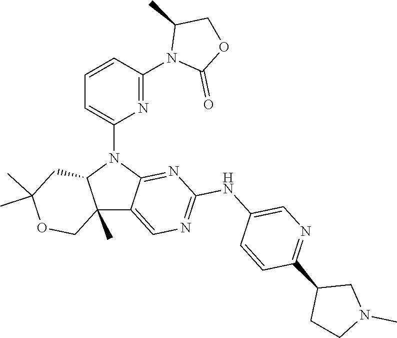 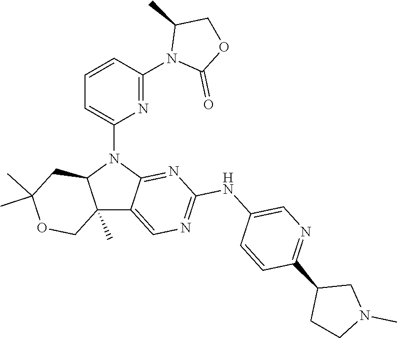 | 264, 265 | 1H NMR (400 MHz, DMSO-d6) δ 9.47 (s, 1H), 8.75 (d, J = 2.8 Hz, 1H), 8.39 (d, J = 8.4 Hz, 1H), 8.15-8.09 (m, 1H), 8.07 (s, 1H), 7.83 (t, J = 8.4 Hz, 1H), 7.68 (d, J = 8.0 Hz, 1H), 7.24 (d, J = 8.4 Hz, 1H), 4.84-4.74 (m, 1H), 4.57 (t, J = 8.4 Hz, 1H), 4.54-4.46 (m, 1H), 4.14- 4.07 (m, 1H), 3.86-3.66 (m, 2H), 3.53- 3.32 (m, 1H), 2.96-2.87 (m, 1H), 2.75- 2.55 (m, 2H), 2.49-2.32 (m, 2H), 2.29 (s, 3H), 2.25-2.10 (m, 1H), 2.05-1.93 (m, 1H), 1.65-1.55 (m, 1H), 1.48 (d, J = 6.0 Hz, 3H), 1.30 (s, 3H), 1.26 (s, 3H), 1.07 (s, 3H). 1H NMR (400 MHz, DMSO-d6) δ 9.49 (s, 1H), 8.75 (d, J = 2.4 Hz, 1H), 8.39 (d, J = 8.1 Hz, 1H), 8.16-8.06 (m, 2H), 7.84 (t, J = 8.1 Hz, 1H), 7.67 (d, J = 8.1 Hz, 1H), 7.24 (d, J = 8.7 Hz, 1H), 4.99- 4.88 (m, 1H), 4.74-4.64 (m, 1H), 4.59- 4.49 (m, 1H), 4.17-4.08 (m, 1H), 3.92- 3.65 (m, 2H), 3.55-3.35 (m, 1H), 2.98- 2.86 (m, 1H), 2.76-2.66 (m, 2H), 2.45- 2.40 (m, 1H), 2.29 (s, 3H), 2.23-2.10 (m, 2H), 2.05-1.93 (m, 1H), 1.62-1.50 (m, 1H), 1.48 (d, J = 6.0 Hz, 3H), 1.28 (s, 3H), 1.25 (s, 3H), 1.07 (s, 3H). | LCMS (ES, m/z): 571 [M + H]+, Rt 0.642 min LCMS (ES, m/z): 571 [M + H]+, Rt 0.650 min. | Column: CHIRALPAK IE, 2 × 25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 30% B to 30% B in 20 min; Wave Length: 254/220 nm; RT1(min): 14.09; RT2(min): 17.16 |
| 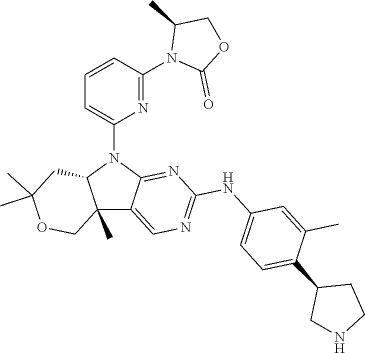 [SMILES: Cc1cc(Nc2ncc3c(n2)N(c2cccc(N4C(=O)OC[C@@H]4C)n2)[C@H]2CC(C)(C)OC[C@@]32C)ccc1[C@H]1CCNC1] 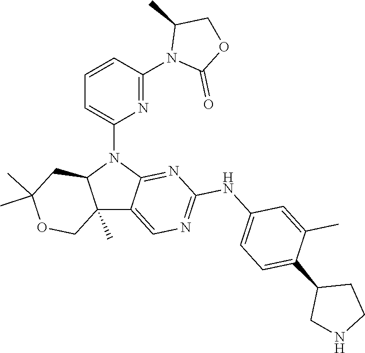 [SMILES: Cc1cc(Nc2ncc3c(n2)N(c2cccc(N4C(=O)OC[C@@H]4C)n2)[C@@H]2CC(C)(C)OC[C@]32C)ccc1[C@H]1CCNC1] | 266, 267 | 1H NMR (300 MHz, DMSO-d6) δ 9.31- 9.27 (m, 1H), 8.42 (d, J = 8.1 Hz, 1H), 8.06 (s, 1H), 7.82 (t, J = 8.4 Hz, 1H), 7.68 (d, J = 7.8 Hz, 1H), 7.61-7.58 (m, 1H), 7.44 (d, J = 8.1 Hz, 1H), 7.20-7.15 (m, 1H), 4.83-4.77 (m, 1H), 4.61-4.48 (m, 2H), 4.13-4.09 (m, 1H), 3.84-3.80 (m, 1H), 3.71-3.64 (m, 1H), 3.29-3.19 (m, 1H), 3.06-2.92 (m, 2H), 2.74-2.64 (m, 2H), 2.39-2.38 (m, 1H), 2.35-2.28 (m, 3H), 2.17-1.87 (m, 1H), 1.75-1.56 (m, 2H), 1.49 (d, J = 6.0 Hz, 3H), 1.30 (s, 3H), 1.25 (s, 3H), 1.08 (s, 3H). 1H NMR (300 MHz, DMSO-d6) δ 9.31- 9.27 (m, 1H), 8.42 (d, J = 8.1 Hz, 1H), 8.07 (s, 1H), 7.83 (t, J = 8.1 Hz, 1H), 7.66 (d, J = 7.8 Hz, 1H), 7.61-7.58 (m, 1H), 7.44 (d, J = 8.7 Hz, 1H), 7.18 (d, J = 8.1 Hz, 1H), 4.96-4.91 (m, 1H), 4.71- 4.66 (m, 1H), 4.55 (t, J = 8.4 Hz, 1H), 4.15-4.11 (m, 1H), 3.88-3.84 (m, 1H), 3.71-3.67 (m, 1H), 3.25-3.19 (m, 1H), 3.06-2.92 (m, 2H), 2.74-2.64 (m, 2H), 2.28 (s, 3H), 2.19-2.08 (m, 2H), 1.77- 1.65 (m, 1H), 1.59-1.48 (m, 1H), 1.39 (d, J = 6.0 Hz, 3H), 1.27 (s, 3H), 1.25 (s, 3H), 1.07 (s, 3H). | LCMS (ES, m/z): 570 [M + H]+, Rt 0.686 min. LCMS (ES, m/z): 570 [M + H]+, Rt 0.690 min. | Column: CHIRALPAK IE, 2 × 25 cm, 5 μm; Mobile Phase A: Hex: DCM = 3:1 (0.5% 2M NH3-MeOH)- HPLC, Mobile Phase B: EtOH- HPLC(15% to 15% in 19 min); Flow rate: 20 mL/min; Wave Length: 220/254 nm; RT1(min): 13.70; RT2(min): 16.57 |
| 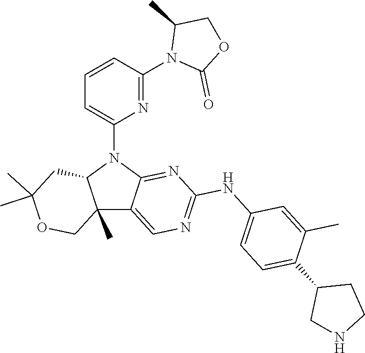 [SMILES: Cc1cc(Nc2ncc3c(n2)N(c2cccc(N4C(=O)OC[C@@H]4C)n2)[C@H]2CC(C)(C)OC[C@@]32C)ccc1[C@@H]1CCNC1] 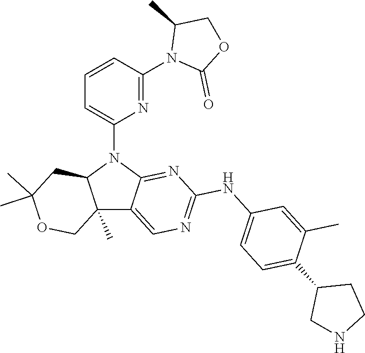 [SMILES: Cc1cc(Nc2ncc3c(n2)N(c2cccc(N4C(=O)OC[C@@H]4C)n2)[C@@H]2CC(C)(C)OC[C@]32C)ccc1[C@@H]1CCNC1] | 268, 269 | 1H NMR (400 MHz, DMSO-d6) δ 9.25 (s, 1H), 8.43 (d, J = 8.0 Hz, 1H), 8.05 (s, 1H), 7.81 (t, J = 8.0 Hz, 1H), 7.68-7.66 (m, 1H), 7.60-7.57 (m, 1H), 7.44-7.42 (m, 1H), 7.18-7.16 (m, 1H), 4.82-4.77 (m, 1H), 4.57 (t, J = 8.4 Hz, 1H), 4.51- 4.48 (m, 1H), 4.12-4.09 (m, 1H), 3.83- 3.80 (m, 1H), 3.70-3.67 (m, 1H), 3.30- 3.26 (m, 1H), 3.01-3.16 (m, 1H), 3.03- 2.90 (m, 2H), 2.67-2.61 (m, 1H), 2.38- 2.31 (m, 4H), 2.15-2.06 (m, 1H), 1.73- 1.56 (m, 2H), 1.49 (d, J = 6.4 Hz, 3H), 1.29 (m, 3H), 1.26 (m, 3H), 1.08 (s, 3H). 1H NMR (400 MHz, DMSO-d6) δ 9.24 (s, 1H), 8.43 (d, J = 8.0 Hz, 1H), 8.06 (s, 1H), 7.82 (t, J = 8.4 Hz, 1H), 7.66-7.65 (m, 1H), 7.57-7.56 (m, 1H), 7.48-7.41 (m, 1H), 7.18-7.16 (m, 1H), 4.94-4.91 (m, 1H), 4.70-4.66 (m, 1H), 4.57 (t, J = 8.0 Hz, 1H), 4.14-4.11 (m, 1H), 3.87- 3.84 (m, 1H), 3.70-3.67 (m, 1H), 3.53- 3.42 (m, 1H), 3.21-3.14 (m, 1H), 3.02- 2.88 (m, 2H), 2.63-2.59 (m, 1H), 2.33- 2.31 (m, 3H), 2.18-2.05 (m, 2H), 1.72- 1.63 (m, 1H), 1.58-1.52 (m, 1H), 1.40 (d, J = 6.0 Hz, 3H), 1.26 (s, 3H), 1.25 (s, 3H), 1.06 (s, 3H). | LCMS (ES, m/z): 570 [M + H]+. Rt 0.683 min. LCMS (ES, m/z): 570 [M + H]+. Rt 0.679 min. | Column: CHIRALPAK IE, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 15% B to 15% B in 22 min; Wave Length: 220/254 nm; RT1(min): 15.27; RT2(min): 18.65; Sample Solvent: EtOH- HPLC; |
| 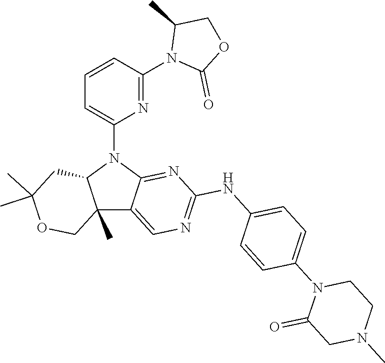 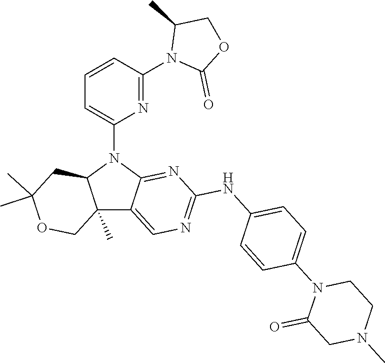 | 270, 271 | 1H NMR (400 MHz, DMSO-d6) δ 9.47 (s, 1H), 8.42 (d, J = 8.0 Hz, 1H), 8.07 (s, 1H), 7.84 (t, J = 8.0 Hz, 1H), 7.76-7.73 (m, 2H), 7.68 (d, J = 8.0 Hz, 1H), 7.23 (d, J = 8.8 Hz, 2H), 4.80-4.78 (m, 1H), 4.59-4.55 (m, 1H), 4.52-4.48 (m, 1H), 4.12-4.09 (m, 1H), 3.84-3.81 (m, 1H), 3.70-3.61 (m, 3H), 3.09 (s, 2H), 2.73- 2.67 (m, 2H), 2.37-2.29 (m, 4H), 1.63- 1.57 (m, 1H), 1.48 (d, J = 6.4 Hz, 3H), 1.30 (s, 3H), 1.26 (s, 3H), 1.07 (s, 3H). 1H NMR (400 MHz, DMSO-d6) δ 9.47 (s, 1H), 8.42 (d, J = 8.4 Hz, 1H), 8.08 (s, 1H), 7.85 (t, J = 8.4 Hz, 1H), 7.75 (d, J = 8.8 Hz, 2H), 7.66 (d, J = 8 Hz, 1H), 7.23 (d, J = 8.8 Hz, 2H), 4.95-4.91 (m, 1H), 4.70-4.67 (m, 1H), 4.56-4.52 (m, 1H), 4.13-4.11(m, 1H), 3.88-3.85 (m, 1H), 3.70-3.67 (m, 1H), 3.64-3.61 (m, 2H), 3.09 (s, 2H), 2.73-2.67 (m, 2H), 2.29 (s, 3H), 2.17-2.13 (m, 1H), 1.59- 1.53 (m, 1H), 1.39 (d, J = 6.0 Hz, 3H), 1.27 (s, 3H), 1.24 (s, 3H), 1.06 (s, 3H). | LCMS (ES, m/z): 599 [M + H]+. Rt 1.236 min. LCMS (ES, m/z): 599 [M + H]+. Rt 0.636 min. | Column: Chiral ART Cellulose- SA, 2*25 cm, 5 μm; Mobile Phase A: MtBE(0.5% 2M NH3-MeOH)- HPLC, Mobile Phase B: EtOH- HPLC; Flow rate: 20 mL/min; Gradient: 30% B to 30% B in 14 min; Wave Length: 254/220 nm; RT1(min): 8.65; RT2(min): 11.78 |
| 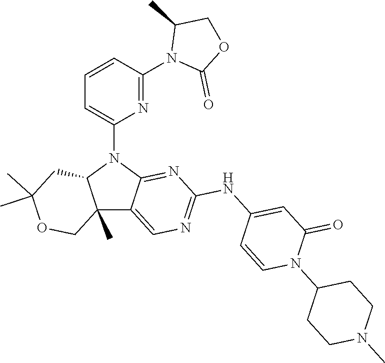 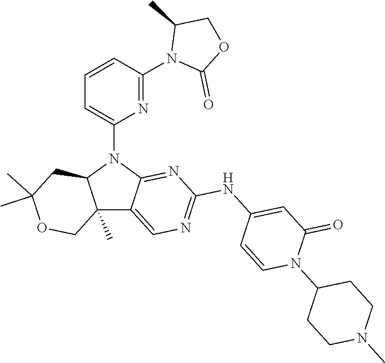 | 272, 273 | 1H NMR (300 MHz, DMSO-d6) δ 9.64 (s, 1H), 8.37 (d, J = 8.1 Hz, 1H), 8.15 (s, 1H), 7.85 (t, J = 8.1 Hz, 1H), 7.69 (d, J = 8.1 Hz, 1H), 7.57 (d, J = 8.1 Hz, 1H), 7.10 (d, J = 2.4 Hz, 1H), 6.65-6.35 (m, 1H), 4.89-4.72 (m, 1H), 4.69-4.43 (m, 3H), 4.16-4.06 (m, 1H), 3.89-3.79 (m, 1H), 3.75-3.65 (m, 1H), 2.93-2.83 (m, 2H), 2.32-2.29 (m, 1H), 2.20 (s, 3H), 2.07-1.94 (m, 2H), 1.90-1.73 (m, 2H), 1.69-1.55 (m, 3H), 1.48 (d, J = 6.3 Hz, 3H), 1.31 (s, 3H), 1.25 (s, 3H), 1.07 (s, 3H) 1H NMR (300 MHz, DMSO-d6) δ 9.64 (s, 1H), 8.37 (d, J = 8.1 Hz, 1H), 8.15 (s, 1H), 7.85 (t, J = 8.1 Hz, 1H), 7.69 (d, J = 8.1 Hz, 1H), 7.57 (d, J = 7.5 Hz, 1H), 7.10 (d, J = 2.4 Hz, 1H), 6.65-6.35 (m, 1H), 5.08-4.72 (m, 1H), 4.80-4.50(m, 3H), 4.16-4.06 (m, 1H), 3.95-3.79 (m, 1H), 3.79-3.65 (m, 1H), 2.93-2.81 (m, 2H), 2.28-2.18 (m, 4H), 2.07-1.85 (m, 4H), 1.71-1.55 (m, 3H), 1.38 (d, J = 6.3 Hz, 3H), 1.28 (s, 3H), 1.24 (s, 3H), 1.05 (s, 3H). | LCMS (ES, m/z): 601 [M + H]+. Rt 0.683 min. LCMS (ES, m/z): 601 [M + H]+. Rt 0.675 min. | Column: CHIRALPAK IG, 2*25 cm, 5 μm; Mobile Phase A: Hex: DCM = 3:1 (0.5% 2M NH3-MeOH)- HPLC, Mobile Phase B: EtOH- HPLC; Flow rate: 18 mL/min; Gradient: 50% B to 50% B in 20 min; Wave Length: 254/220 nm; RT1(min): 9.7; RT2(min): 14.59; |
| 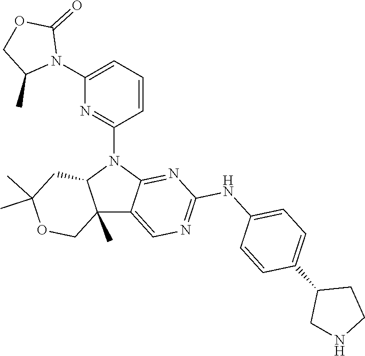 [SMILES: C[C@H]1COC(=O)N1c1cccc(N2c3nc(Nc4ccc([C@@H]5CCNC5)cc4)ncc3[C@]3(C)COC(C)(C)C[C@H]23)n1] 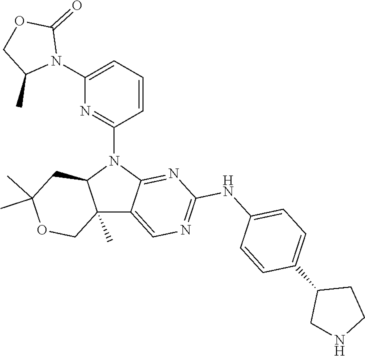 [SMILES: C[C@H]1COC(=O)N1c1cccc(N2c3nc(Nc4ccc([C@@H]5CCNC5)cc4)ncc3[C@@]3(C)COC(C)(C)C[C@@H]23)n1] | 274, 275 | 1H NMR (300 MHz, DMSO-d6) δ 9.39- 9.29 (m, 1H), 8.43 (d, J = 8.4 Hz, 1H), 8.05 (s, 1H), 7.83 (t, J = 8.1 Hz, 1H), 7.73-7.60 (m, 3H), 7.26-7.14 (m, 2H), 4.86-4.74 (m, 1H), 4.63-4.53 (m, 1H), 4.52-4.44 (m, 1H), 4.15-4.05 (m, 1H), 3.82-3.62 (m, 2H), 3.25-3.10 (m, 1H), 3.10-3.02 (m, 1H), 3.02-2.82 (m, 2H), 2.68-2.55 (m, 1H), 2.42-2.30 (m, 1H), 2.20-2.05 (m, 1H), 1.80-1.55 (m, 2H), 1.49 (d, J = 8.0 Hz, 3H), 1.30 (s, 3H), 1.26 (s, 3H), 1.08 (s, 3H). 1H NMR (300 MHz, DMSO-d6) δ 9.39- 9.29 (m, 1H), 8.43 (d, J = 8.4 Hz, 1H), 8.05 (s, 1H), 7.84 (t, J = 8.1 Hz, 1H), 7.73-7.60 (m, 3H), 7.26-7.14 (m, 2H), 5.00-4.88 (m, 1H), 4.73-4.63 (m, 1H), 4.60-4.48 (m, 1H), 4.17-4.08 (m, 1H), 3.92-3.64 (m, 2H), 3.10-3.02 (m, 1H), 3.02-2.84 (m, 2H), 2.67-2.55 (m, 1H), 2.42-2.30 (m, 1H), 2.20-2.00 (m, 2H), 1.77-1.60 (m, 1H), 1.60-1.50 (m, 1H), 1.39 (d, J = 8.0 Hz, 3H), 1.27 (s, 3H), 1.25 (s, 3H), 1.07 (s, 3H). | LCMS (ES, m/z): 556 [M + H]+, Rt 0.675 min. LCMS (ES, m/z): 556 [M + H]+, Rt 0.683 min. | Column: CHIRALPAK IG, 2 × 25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 30% B to 30% B in 21.5 min; Wave Length: 220/254 nm; RT1(min): 12.53; RT2(min): 19.11; |
| 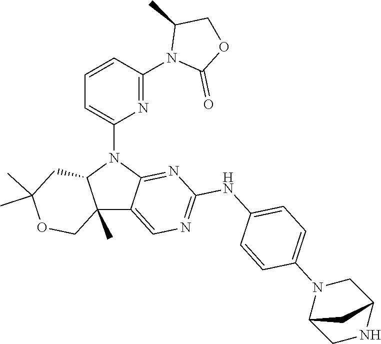 [SMILES: C[C@H]1COC(=O)N1c1cccc(N2c3nc(Nc4ccc(N5C[C@H]6C[C@@H]5CN6)cc4)ncc3[C@]3(C)COC(C)(C)C[C@H]23)n1] 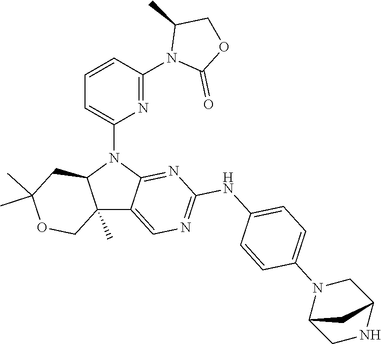 [SMILES: C[C@H]1COC(=O)N1c1cccc(N2c3nc(Nc4ccc(N5C[C@H]6C[C@@H]5CN6)cc4)ncc3[C@@]3(C)COC(C)(C)C[C@@H]23)n1] | 276, 277 | 1H NMR (300 MHz, DMSO-d6) δ 8.97 (s, 1H), 8.42 (d, J = 8.1 Hz, 1H), 7.98 (s, 1H), 7.79 (t, J = 8.1 Hz, 1H), 7.65 (d, J = 7.8 Hz, 1H), 7.44 (d, J = 8.7 Hz, 2H), 6.53 (d, J = 8.7 Hz, 2H), 4.89-4.73 (m, 1H), 4.57 (t, J = 8.4 Hz, 1H), 4.49-4.45 (m, 1H), 4.29-4.26 (m, 1H), 4.13-4.09 (m, 1H), 3.82-3.78 (m, 1H), 3.69-3.65 (m, 1H), 3.61-3.56 (m, 1H), 3.53-3.48 (m, 1H), 2.86 (s, 3H), 2.38-2.32 (m, 1H), 1.81-1.77 (m, 1H), 1.71-1.54 (m, 2H), 1.48 (d, J = 6.0 Hz, 3H), 1.28 (s, 3H), 1.26 (s, 3H), 1.08 (s, 3H). 1H NMR (300 MHz, DMSO-d6) δ 9.03 (s, 1H), 8.42 (d, J = 7.8 Hz, 1H), 8.00 (s, 1H), 7.81 (t, J = 8.1 Hz, 1H), 7.64 (d, J = 7.8 Hz, 1H), 7.49 (d, J = 8.7 Hz, 2H), 6.59 (d, J = 9.0 Hz, 2H), 4.95-4.92 (m, 1H), 4.69-4.64 (m, 1H), 4.55 (t, J = 8.1 Hz, 1H), 4.46-4.41 (m, 1H), 4.15-4.11 (m, 1H), 3.98-3.93 (m, 1H), 3.87-3.83 (m, 1H), 3.70-3.66 (m, 1H), 3.61-3.50 (m, 1H), 3.04-2.95 (m 3H), 2.18-2.12 (m, 1H), 1.98-1.92 (m, 1H), 1.81-1.77 (m, 1H), 1.57-1.49 (m, 1H), 1.38 (d, J = 6.3 Hz, 3H), 1.25 (s, 3H), 1.24 (s, 3H), 1.06 (s, 3H). | LCMS (ES, m/z): 583 [M + H]+, Rt 0.694 min. LCMS (ES, m/z): 583 [M + H]+, Rt 0.710 min. | Column: CHIRALPAK IA, 2*25 cm, 5 μm; Mobile Phase A: MtBE(0.5% 2M NH3- MeOH)-HPLC, Mobile Phase B: EtOH-HPLC; Flow rate: 20 mL/min; Gradient: 30% B to 30% B in 9.75 min; Wave Length: 220/254 nm; RT1(min): 6.42; RT2(min): 8.26; |
| 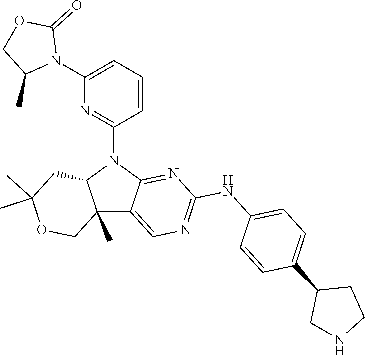 [SMILES: C[C@H]1COC(=O)N1c1cccc(N2c3nc(Nc4ccc([C@H]5CCNC5)cc4)ncc3[C@]3(C)COC(C)(C)C[C@H]23)n1] 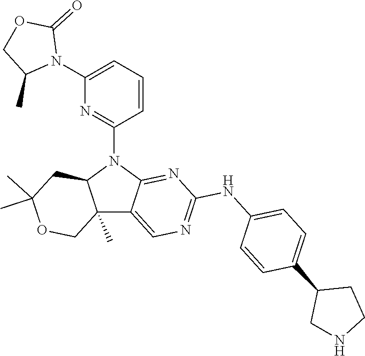 [SMILES: C[C@H]1COC(=O)N1c1cccc(N2c3nc(Nc4ccc([C@H]5CCNC5)cc4)ncc3[C@@]3(C)COC(C)(C)C[C@@H]23)n1] | 278, 279 | 1H NMR (300 MHz, DMSO-d6) δ 9.31 (s, 1H), 8.43 (d, J = 8.1 Hz, 1H), 8.04 (s, 1H), 7.83 (t, J = 8.1 Hz, 1H), 7.71-7.62 (m, 3H), 7.19 (d, J = 8.4 Hz, 2H), 4.88- 4.72 (m, 1H), 4.58 (t, J = 8.1 Hz, 1H), 4.51-4.46 (m, 1H), 4.12-4.08 (m, 1H), 3.83-3.79 (m, 1H), 3.70-3.66 (m, 1H), 3.51-3.43 (m, 1H), 3.21-3.15 (m, 1H), 3.09-2.93 (m, 2H), 2.65-2.61 (m, 1H), 2.38-2.32 (m, 1H), 2.24-2.03 (m, 1H), 1.76-1.52 (m, 2H), 1.48 (d, J = 6.0 Hz, 3H), 1.28 (s, 3H), 1.25 (s, 3H), 1.07 (s, 3H) 1H NMR (300 MHz, DMSO-d6) δ 9.31 (s, 1H), 8.43 (d, J = 8.1 Hz, 1H), 8.06 (s, 1H), 7.84 (t, J = 8.1 Hz, 1H), 7.67-7.63 (m, 3H), 7.18 (d, J = 8.7 Hz, 2H), 5.00- 4.86 (m, 1H), 4.71-4.66 (m, 1H), 4.55 (t, J = 8.1 Hz, 1H), 4.15-4.11 (m, 1H), 3.88-3.84 (m, 1H), 3.71-3.67 (m, 1H), 3.21-2.92 (m, 4H), 2.65-2.62 (m, 1H), 2.19-2.10 (m, 2H), 1.69-1.63 (m, 1H), 1.59-1.55 (m, 1H), 1.39 (d, J = 6.0 Hz, 3H), 1.27 (s, 3H), 1.25 (s, 3H), 1.07 (s, 3H) | LCMS (ES, m/z): 556 [M + H]+, Rt 0.687 min. LCMS (ES, m/z): 556 [M + H]+, Rt 0.686 min. | Column: CHIRALPAK IG, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 50% B to 50% B in 12 min; Wave Length: 220/254 nm; RT1(min): 9.26; RT2(min): 10.89; |
| 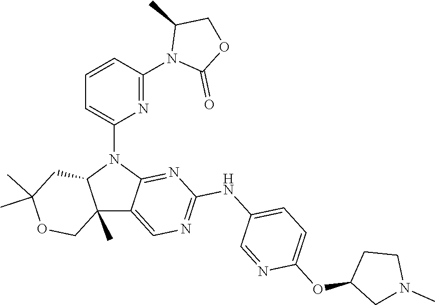 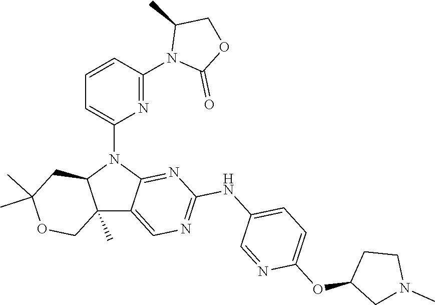 | 280, 281 | 1H NMR (300 MHz, DMSO-d6) δ 9.30 (s, 1H), 8.41-8.34 (m, 2H), 8.04-8.01 (m, 2H), 7.81 (t, J = 8.1 Hz, 1H), 7.67 (d, J = 8.1 Hz, 1H), 6.79 (d, J = 9 Hz, 1H), 5.30-5.29 (m, 1H), 4.96-4.91 (m, 1H), 4.70-4.66 (m, 1H), 4.57-4.52 (m, 1H), 4.13-4.08 (m, 1H), 3.83-3.79 (m, 1H), 3.70-3.66 (m, 1H), 2.81-2.75 (m, 1H), 2.66-2.55 (m, 2H), 2.37-2.35 (m, 2H), 2.26-2.12 (m, 4H), 1.83-1.74 (m, 1H), 1.58-1.51 (m, 1H), 1.48 (d, J = 6.3 Hz, 3H), 1.29 (s, 3H), 1.25 (s, 3H), 1.07 (s, 3H). 1H NMR (300 MHz, DMSO-d6) δ 9.30 (s, 1H), 8.41-8.34 (m, 2H), 8.04-8.01 (m, 2H), 7.82 (t, J = 8.1 Hz, 1H), 7.66 (d, J = 7.8 Hz, 1H), 6.79 (d, J = 9.0 Hz, 1H), 5.29-5.28 (m, 1H), 4.96-4.91 (m, 1H), 4.68-4.65 (m, 1H), 4.57-4.52 (m, 1H), 4.14-4.11 (m, 1H), 3.87-3.83 (m, 1H), 3.71-3.67 (m, 1H), 2.81-2.76 (m, 1H), 2.67-2.56 (m, 2H), 2.37-2.34 (m, 1H), 2.28-2.21 (m, 4H), 2.22-2.12 (m, 1H), 1.83-1.75 (m, 1H), 1.57-1.51 (m, 1H), 1.39 (d, J = 6.0 Hz, 3H), 1.26 (s, 3H), 1.24 (s, 3H), 1.06 (s, 3H). | LCMS (ES, m/z): 587 [M + H]+. Rt 0.671 min. LCMS (ES, m/z): 587 [M + H]+. Rt 0.669 min. | Column: CHIRAL ART Cellulose-SC, 2*25 cm, 5 μm; Mobile Phase A: Hex: DCM = 3:1 (0.5% 2M NH3-MeOH)- HPLC, Mobile Phase B: IPA- HPLC; Flow rate: 20 mL/min; Gradient: 30% B to 30% B in 13 min; Wave Length: 220/254 nm; RT1(min): 8.94; RT2(min): 11.56; |
| 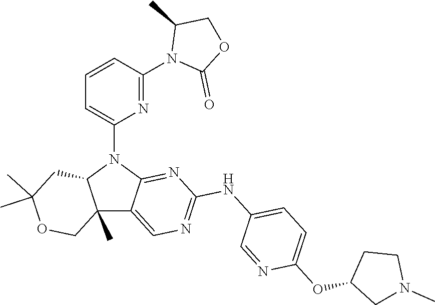 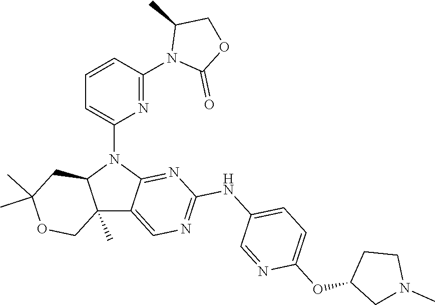 | 282, 283 | 1H NMR (300 MHz, DMSO-d6) δ 9.30 (s, 1H), 8.42-8.35 (m, 2H), 8.05-8.01 (m, 2H), 7.81 (t, J = 8.1 Hz, 1H), 7.68 (d, J = 7.8 Hz, 1H), 6.79 (d, J = 9.0 Hz, 1H), 5.34-5.27 (m, 1H), 4.85-4.76 (m, 1H), 4.57 (t, J = 8.4 Hz, 1H), 4.51-4.47 (m, 1H), 4.13-4.09 (m, 1H), 3.84-3.80 (m, 1H), 3.71-3.67 (m, 1H), 2.82-2.76 (m, 1H), 2.70-2.57 (m, 2H), 2.40-2.33 (m, 2H), 2.29-2.24 (m, 4H), 1.82-1.77 (m, 1H), 1.63-1.55 (m, 1H), 1.49 (d, J = 6.0 Hz, 3H), 1.29 (s, 3H), 1.26 (s, 3H), 1.08 (s, 3H). 1H NMR (300 MHz, DMSO-d6) δ 9.30 (s, 1H), 8.41-8.35 (m, 2H), 8.05-8.01 (m, 2H), 7.82 (t, J = 8.1 Hz, 1H), 7.67 (d, J = 8.1 Hz, 1H), 6.79 (d, J = 9.0 Hz, 1H), 5.32-5.28 (m, 1H), 4.95-4.91 (m, 1H), 4.70-4.66 (m, 1H), 4.55 (t, J = 8.4 Hz, 1H), 4.15-4.09 (m, 1H), 3.88-3.84 (m, 1H), 3.71-3.67 (m, 1H), 2.81-2.76 (m, 1H), 2.70-2.56 (m, 2H), 2.40-2.26 (m, 5H), 2.18-2.12 (m, 1H), 1.83-1.74 (m, 1H), 1.58-1.51 (m, 1H), 1.40 (d, J = 6.3 Hz, 3H), 1.26 (s, 3H), 1.25 (s, 3H), 1.07 (s, 3H). | LCMS (ES, m/z): 587 [M + H]+. Rt 0.675 min LCMS (ES, m/z): 587 [M + H]+. Rt 0.676 min. | Column: CHIRALPAK IG, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 15% B to 15% B in 19 min; Wave Length: 220/254 nm; RT1(min): 14.49; RT2(min): 16.99; |
| 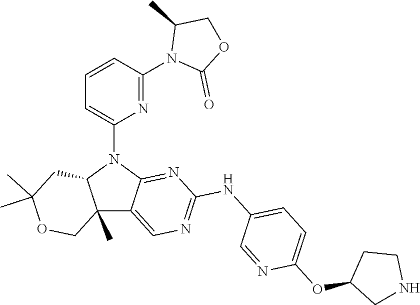 [SMILES: C[C@H]1COC(=O)N1c1cccc(N2c3nc(Nc4ccc(O[C@H]5CCNC5)nc4)ncc3[C@]3(C)COC(C)(C)C[C@H]23)n1] 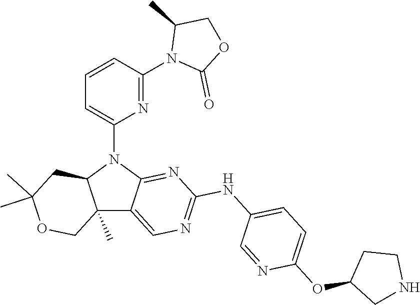 [SMILES: C[C@H]1COC(=O)N1c1cccc(N2c3nc(Nc4ccc(O[C@H]5CCNC5)nc4)ncc3[C@@]3(C)COC(C)(C)C[C@@H]23)n1] | 284, 285 | 1H NMR (300 MHz, DMSO-d6) δ 9.30 (s, 1H), 8.42-8.34 (m, 2H), 8.04-8.00 (m, 2H), 7.81 (t, J = 8.4 Hz, 1H), 7.67 (d, J = 8.1 Hz, 1H), 6.78 (d, J = 9.0 Hz, 1H), 5.32-5.30 (m, 1H), 4.82-4.74 (m, 1H), 4.60-4.46 (m, 2H), 4.12-4.08 (m, 1H), 3.83-3.79 (m, 1H), 3.70-3.66 (m, 1H), 3.11-3.05 (m, 1H), 2.93-2.78 (m, 3H), 2.36-2.28 (m, 1H), 2.09-1.96 (m, 1H), 1.82-1.70 (m, 1H), 1.63-1.55 (m, 1H), 1.48 (d, J = 6.3 Hz, 3H), 1.29 (s, 3H), 1.26 (s, 3H), 1.08 (s, 3H). 1H NMR (300 MHz, DMSO-d6) δ 9.30 (s, 1H), 8.41-8.34 (m, 2H), 8.04-8.01 (m, 2H), 7.82 (t, J = 8.1 Hz, 1H), 7.65 (d, J = 7.8 Hz, 1H), 6.78 (d, J = 9.0 Hz, 1H), 5.31-5.30 (m, 1H), 4.93 (s, 1H), 4.70-4.65 (m, 1H), 4.57-4.51 (m, 1H), 4.13-4.10 (m, 1H), 3.87-3.83 (m, 1H), 3.70-3.66 (m, 1H), 3.11-3.05 (m, 1H), 2.93-2.78 (m, 3H), 2.16-2.11 (m, 2H), 1.82-1.74 (m, 1H), 1.58-1.51 (m, 1H), 1.39 (d, J = 6.0 Hz, 3H), 1.26 (s, 3H), 1.24 (s, 3H), 1.04 (s, 3H). | LCMS (ES, m/z): 573 [M + H]+. Rt 0.660 min. LCMS (ES, m/z): 573 [M + H]+. Rt 0.659 min. | Column: CHIRAL ART Cellulose-SC, 2*25 cm, 5 μm; Mobile Phase A: Hex: DCM = 3:1 (0.5% 2M NH3-MeOH)- HPLC, Mobile Phase B: IPA- HPLC; Flow rate: 20 mL/min; Gradient: 30% B to 30% B in 13 min; Wave Length: 220/254 nm; RT1(min): 8.94; RT2(min): 11.56; |
| 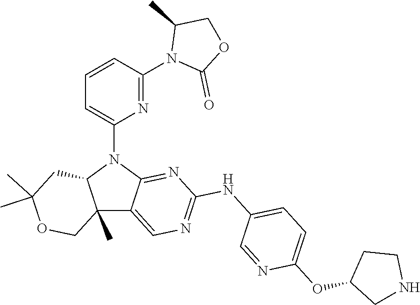 [SMILES: C[C@H]1COC(=O)N1c1cccc(N2c3nc(Nc4ccc(O[C@@H]5CCNC5)nc4)ncc3[C@]3(C)COC(C)(C)C[C@H]23)n1] 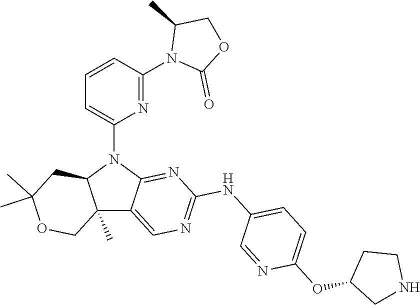 [SMILES: C[C@H]1COC(=O)N1c1cccc(N2c3nc(Nc4ccc(O[C@@H]5CCNC5)nc4)ncc3[C@@]3(C)COC(C)(C)C[C@@H]23)n1] | 286, 287 | 1H NMR (300 MHz, DMSO-d6) δ 9.30 (s, 1H), 8.42-8.35 (m, 2H), 8.05-8.01 (m, 2H), 7.81 (t, J = 8.4 Hz, 1H), 7.68 (d, J = 8.1 Hz, 1H), 6.78 (d, J = 9.0 Hz, 1H), 5.35-5.29 (m, 1H), 4.83-4.76 (m, 1H), 4.60-4.47 (m, 2H), 4.13-4.09 (m, 1H), 3.84-3.80 (m, 1H), 3.71-3.67 (m, 1H), 3.12-3.06 (m, 1H), 2.96-2.73 (m, 3H), 2.38-2.32 (m, 1H), 2.09-1.97 (m, 1H), 1.82-1.74 (m, 1H), 1.63-1.55 (m, 1H), 1.49 (d, J = 6.3 Hz, 3H), 1.29 (s, 3H), 1.26 (s, 3H), 1.08 (s, 3H). 1H NMR (300 MHz, DMSO-d6) δ 9.30 (s, 1H), 8.42-8.35 (m, 2H), 8.04-8.01 (m, 2H), 7.81 (t, J = 8.1 Hz, 1H), 7.67 (d, J = 7.8 Hz, 1H), 6.78 (d, J = 5.7 Hz, 1H), 5.33-5.28 (m, 1H), 4.97-4.88 (m, 1H), 4.71-4.66 (m, 1H), 4.55 (t, J = 8.4 Hz, 1H), 4.15-4.11 (m, 1H), 3.88-3.84 (m, 1H), 3.71-3.67 (m, 1H), 3.12-3.06 (m, 1H), 2.96-2.73 (m, 3H), 2.18-2.12 (m, 1H), 2.05-1.98 (m, 1H), 1.81-1.70 (m, 1H), 1.58-1.51 (m, 1H), 1.39 (d, J = 6.0 Hz, 3H), 1.26 (m, 3H), 1.25 (m, 3H), 1.07 (s, 3H). | LCMS (ES, m/z): 573 [M + H]+. Rt 0.659 min. LCMS (ES, m/z): 573 [M + H]+. Rt 1.232 min. | Column: CHIRALPAK IG, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 15% B to 15% B in 19 min; Wave Length: 220/254 nm; RT1(min): 14.49; RT2(min): 16.99 |
| 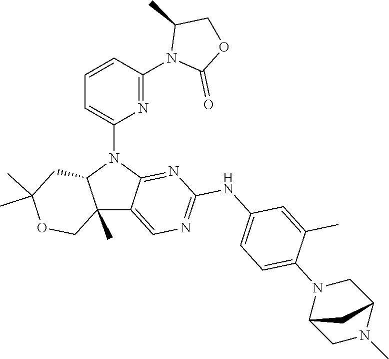 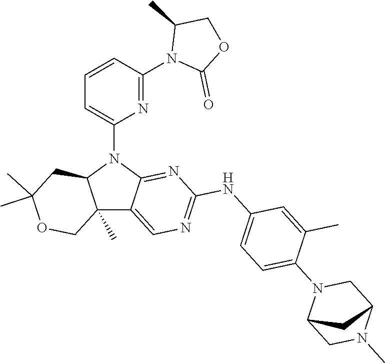 | 288, 289 | 1H NMR (300 MHz, DMSO-d6) δ 9.08 (s, 1H), 8.42 (d, J = 7.8 Hz, 1H), 8.02 (s, 1H), 7.79 (t, J = 8.1 Hz, 1H), 7.65 (d, J = 7.8 Hz, 1H), 7.46 (d, J = 2.1 Hz, 1H), 7.30-7.27 (m, 1H), 6.76 (d, J = 8.7 Hz, 1H), 4.93-4.89 (m, 1H), 4.69-4.64 (m, 1H), 4.57-4.51 (m, 1H), 4.14-4.10 (m, 1H), 3.91-3.87 (m, 2H), 3.83-3.66 (m, 1H), 3.34-3.19 (m, 2H), 2.75-2.68 (m, 2H), 2.22 (s, 3H), 2.16-2.13 (m, 4H), 1.78-1.69 (m, 3H), 1.53-1.49 (m, 1H), 1.39 (d, J = 6.3 Hz, 3H), 1.25 (s, 3H), 1.24 (s, 3H), 1.06 (s, 3H). 1H NMR (300 MHz, DMSO-d6) δ 9.07 (s, 1H), 8.42 (d, J = 8.1 Hz, 1H), 8.00 (s, 1H), 7.79 (t, J = 8.1 Hz, 1H), 7.66 (d, J = 7.8 Hz, 1H), 7.46 (d, J = 2.1 Hz, 1H), 7.29-7.26 (m, 1H), 6.76 (d, J = 8.7 Hz, 1H), 4.93 (s, 1H), 4.60-4.54 (m, 1H), 4.50-4.45 (m, 1H), 4.12-4.08 (m, 1H), 3.91 (s, 1H), 3.82-3.78 (m, 1H), 3.69- 3.65 (m, 1H), 3.25-3.17 (m, 2H), 2.79- 2.68 (m, 2H), 2.33-2.29 (m, 4H), 2.22 (s, 3H), 1.81-1.69 (m, 3H), 1.62-1.57 (m, 1H), 1.48 (d, J = 6.3 Hz, 3H), 1.29 (s, 3H), 1.24 (s, 3H), 1.07 (s, 3H). | LCMS (ES, m/z): 611 [M + H]+. Rt 0.709 min. LCMS (ES, m/z): 611 [M + H]+. Rt 0.710 min. | Column: CHIRALPAK IE, 3*25 cm, 5 μm; Mobile Phase A: Hex: DCM = 3:1 (0.5% 2M NH3-MeOH)- HPLC, Mobile Phase B: EtOH- HPLC; Flow rate: 45 mL/min; Gradient: 15% B to 15% B in 38 min; Wave Length: 220/254 nm; RT1(min): 28; RT2(min): 33.5; |
| 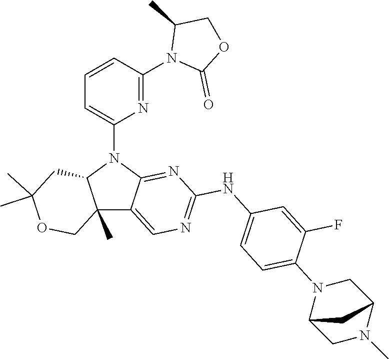 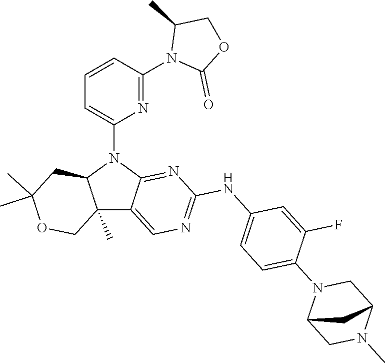 | 290, 291 | 1H NMR (300 MHz, DMSO-d6) δ 9.24 (s, 1H), 8.40 (d, J = 8.1 Hz, 1H), 8.04 (s, 1H), 7.81 (t, J = 8.1 Hz, 1H), 7.69-7.61 (m, 2H), 7.24 (d, J = 8.7 Hz, 1H), 6.73- 6.67 (m, 1H), 4.86-4.72 (m, 1H), 4.60- 4.48 (m, 2H), 4.19-4.09 (m, 2H), 3.83- 3.79 (m, 1H), 3.70-3.66 (m, 1H), 3.43- 3.33 (m, 2H), 3.21-3.18 (m, 1H), 2.87- 2.79 (m, 1H), 2.71-2.63 (m, 1H), 2.41- 2.30 (m, 1H), 2.27 (s, 3H), 1.88-1.83 (m, 1H), 1.77-1.68 (m, 1H), 1.67-1.53 (m, 1H), 1.48 (d, J = 6.0 Hz, 3H), 1.29 (s, 3H), 1.26 (s, 3H), 1.08 (s, 3H). 1H NMR (300 MHz, DMSO-d6) δ 9.25 (s, 1H), 8.40 (d, J = 8.1 Hz, 1H), 8.05 (s, 1H), 7.81 (t, J = 8.1 Hz, 1H), 7.67-7.61 (m, 2H), 7.26-7.23 (m, 1H), 6.73-6.67 (m, 1H), 4.98-4.86 (m, 1H), 4.72-4.58 (m, 2H), 4.19-4.11 (m, 2H), 3.88-3.84 (m, 1H), 3.71-3.67 (m, 1H), 3.43-3.35 (m, 1H), 3.25-3.18 (m, 1H), 2.78-2.71 (m, 1H), 2.67-2.62 (m, 1H), 2.55-2.50 (m, 1H), 2.38-2.32 (m, 1H), 2.27 (s, 3H), 1.88-1.67 (m, 2H), 1.62-1.46 (m, 1H), 1.48 (d, J = 6.3 Hz, 3H), 1.29 (s, 3H), 1.25 (s, 3H), 1.07 (s, 3H). | LCMS (ES, m/z): 615 [M + H]+, Rt 0.706 min. LCMS (ES, m/z): 615 [M + H]+, Rt 0.699 min. | Column: CHIRALPAK IA, 2*25 cm, 20 μm; Mobile Phase A: MtBE (0.5% 2M NH3-MeOH)- HPLC, Mobile Phase B: EtOH- HPLC; Flow rate: 20 mL/min; Gradient: 25% B to 0% B in 13.5 min; Wave Length: 220/254 nm; RT1(min): 6.77; RT2(min): 9.99; Sample Solvent: EtOH- HPLC; Injection Volume: 2.5 mL; Number Of Runs: 2 |
| 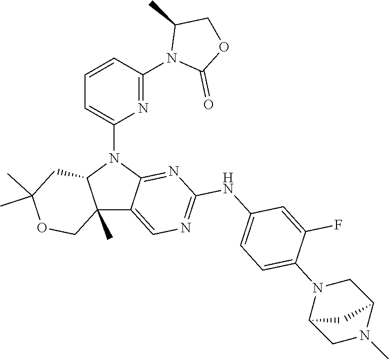 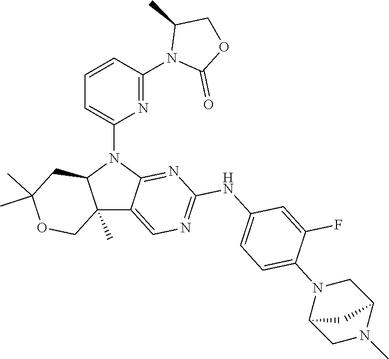 | 292, 293 | 1H NMR (300 MHz, DMSO-d6) δ 9.25 (s, 1H), 8.41 (d, J = 8.1 Hz, 1H), 8.04 (s, 1H), 7.81 (t, J = 8.1 Hz, 1H), 7.69-7.61 (m, 2H), 7.24 (d, J = 9.0 Hz, 1H), 6.73- 6.67 (m, 1H), 4.98-4.77 (m, 1H), 4.60- 4.48 (m, 2H), 4.19-4.16 (m, 1H), 4.13- 4.09 (m, 1H), 3.84-3.80 (m, 1H), 3.70- 3.66 (m, 1H), 3.44-3.41 (m, 1H), 3.22- 3.18 (m, 1H), 2.80-2.74 (m, 1H), 2.66- 2.61 (m, 1H), 2.55-2.51 (m, 1H), 2.39- 2.32 (m, 1H), 2.26 (s, 3H), 1.82-1.78 (m, 1H), 1.75-1.71 (m, 1H), 1.63-1.55 (m, 1H), 1.49 (d, J = 6.0 Hz, 3H), 1.29 (s, 3H), 1.26 (s, 3H), 1.08 (s, 3H). 1H NMR (300 MHz, DMSO-d6) δ 9.25 (s, 1H), 8.40 (d, J = 8.1 Hz, 1H), 8.05 (s, 1H), 7.81 (t, J = 8.3 Hz, 1H), 7.67-7.61 (m, 2H), 7.25-7.22 (m, 1H), 6.73-6.67 (m, 1H), 4.97-4.91 (m, 1H), 4.70-4.65 (m, 1H), 4.55 (t, J = 8.4 Hz, 1H), 4.19- 4.11 (m, 2H), 3.87-3.83 (m, 1H), 3.71- 3.67 (m, 1H), 3.44-3.40 (m, 1H), 3.22- 3.18 (m, 1H), 2.79-2.73 (m, 1H), 2.66- 2.61 (m, 1H), 2.55-2.52 (m, 1H), 2.27 (s, 3H), 2.18-2.12 (m, 1H), 1.86-1.81 (m, 1H), 1.74-1.69 (m, 1H), 1.59-1.51 (m, 1H), 1.39 (d, J = 6.3 Hz, 3H), 1.26 (s, 3H), 1.25 (s, 3H), 1.07 (s, 3H). | LCMS (ES, m/z): 615 [M + H]+, Rt 0.709 min. LCMS (ES, m/z): 615 [M + H]+, Rt 0.708 min. | Column: CHIRALPAK AD-H, 2 × 25 cm, 5 μm; Mobile Phase A: Hex(0.5% 2M NH3-MeOH)- HPLC, Mobile Phase B: EtOH- HPLC(50% to 50% in 22.5 min); Flow rate: 20 mL/min; Wave Length: 220/254 nm; RT1(min): 13.56; RT2(min): 18.08 |
| 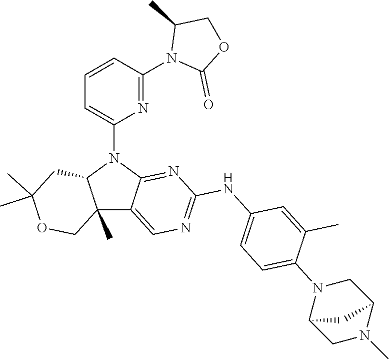 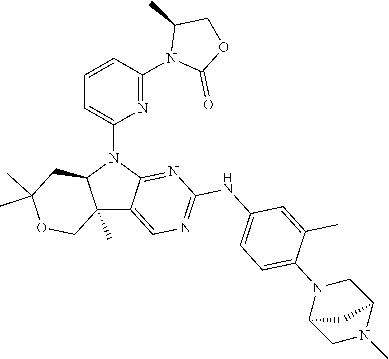 | 294, 295 | 1H NMR (300 MHz, DMSO-d6) δ 9.07 (s, 1H), 8.41 (d, J = 8.4 Hz, 1H), 8.01 (s, 1H), 7.79 (t, J = 8.1 Hz, 1H), 7.65 (d, J = 7.8 Hz, 1H), 7.46 (d, J = 2.4 Hz, 1H), 7.28 (d, J = 8.4 Hz, 1H), 6.75 (d, J = 8.7 Hz, 1H), 4.86-4.74 (m, 1H), 4.57 (t, J = 8.4 Hz, 1H), 4.50-4.47 (m, 1H), 4.12- 4.08 (m, 1H), 3.91 (s, 1H), 3.83-3.79 (m, 1H), 3.69-3.65 (m, 1H), 3.25-3.19 (m, 2H), 2.75-2.68 (m, 2H), 2.34-2.28 (m, 4H), 2.22 (s, 3H), 1.80-1.71 (m, 2H), 1.58-1.51 (m, 1H), 1.48 (d, J = 6.3 Hz, 3H), 1.28 (s, 3H), 1.26-1.15 (m, 4H), 1.08 (s, 3H). 1H NMR (300 MHz, DMSO-d6) δ 9.07 (s, 1H), 8.41 (d, J = 8.1 Hz, 1H), 8.02 (s, 1H), 7.80 (t, J = 8.1 Hz, 1H), 7.64 (d, J = 8.1 Hz, 1H), 7.46 (d, J = 2.4 Hz, 1H), 7.28 (d, J = 8.1 Hz, 1H), 6.76-6.73 (m, 1H), 4.86-4.73 (m, 1H), 4.67-4.64 (m, 1H), 4.54 (t, J = 8.4 Hz, 1H), 4.14-4.10 (m, 1H), 3.91-3.83 (m, 2H), 3.70-3.66 (m, 1H), 3.22-3.20 (m, 2H), 2.75-2.72 (m, 2H), 2.30 (s, 3H), 2.28-2.12 (m, 4H), 1.80-1.71 (m, 2H), 1.54-1.53 (m, 1H), 1.38 (d, J = 6.0 Hz, 3H), 1.28 (s, 3H), 1.26 (s, 3H), 1.25-1.24 (m, 1H), 1.06 (s, 3H). | LCMS (ES, m/z): 611 [M + H]+, Rt 0.712 min. LCMS (ES, m/z): 611 [M + H]+, Rt 0.716 min. | Column: CHIRAL ART Cellulose-SC, 2*25 cm, 5 μm; Mobile Phase A: MtBE(0.5% 2M NH3- MeOH)-HPLC, Mobile Phase B: EtOH-HPLC; Flow rate: 20 mL/min; Gradient: 10% B to 10% B in 11.5 min; Wave Length: 220/254 nm; RT1(min): 6.98; RT2(min): 9.71 |
| 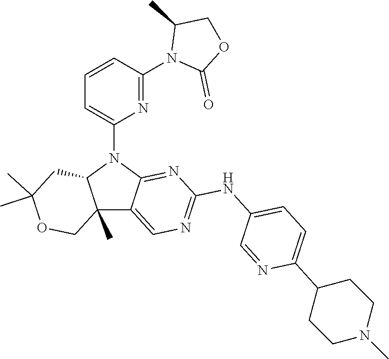 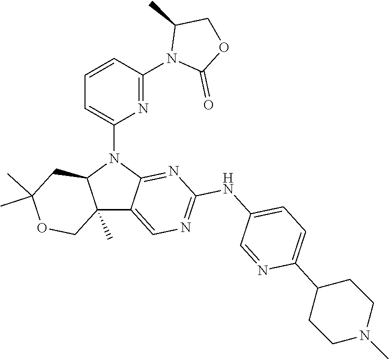 | 296, 297 | 1H NMR (300 MHz, DMSO-d6) δ 9.50 (s, 1H), 8.77 (d, J = 2.7 Hz, 1H), 8.38 (d, J = 8.1 Hz, 1H), 8.17-8.05 (m, 2H), 7.83 (t, J = 8.1 Hz, 1H), 7.68 (d, J = 8.1 Hz, 1H), 7.22 (d, J = 8.4 Hz, 1H), 4.83- 4.77 (m, 1H), 4.63-4.45 (m, 2H), 4.15- 4.05 (m, 1H), 3.87-3.65 (m, 2H), 3.11- 2.98 (m, 2H), 2.69-2.63 (m, 1H), 2.41- 2.29 (m, 6H), 1.93-1.78 (m, 4H), 1.67- 1.53 (m, 1H), 1.48 (d, J = 6.0 Hz, 3H), 1.29 (s, 3H), 1.25 (s, 3H), 1.07 (s, 3H). 1H NMR (300 MHz, DMSO-d6) δ 9.48 (s, 1H), 8.75 (d, J = 2.7 Hz, 1H), 8.38 (d, J = 8.1 Hz, 1H), 8.16-8.07 (m, 2H), 7.83 (t, J = 8.1 Hz, 1H), 7.67 (d, J = 8.1 Hz, 1H), 7.21 (d, J = 8.4 Hz, 1H), 4.97- 4.91 (m, 1H), 4.73-4.65 (m, 1H), 4.54 (t, J = 8.1 Hz, 1H), 4.15-4.08 (m, 1H), 3.92-3.82 (m, 1H), 3.74-3.64 (m, 1H), 2.98-2.88 (m, 2H), 2.65-2.52 (m, 1H), 2.26 (s, 3H), 2.21-2.01 (m, 3H), 1.88- 1.69 (m, 4H), 1.62-1.48 (m, 1H), 1.39 (d, J = 6.0 Hz, 3H), 1.26 (s, 3H), 1.24 (s, 3H), 1.06 (s, 3H). | LCMS (ES, m/z): 585 [M + H] +; RT: 0.630 min. LCMS (ES, m/z): 585 [M + H] +; RT: 0.683 min. | Column: CHIRAL ART Cellulose-SC, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 50% B to 50% B in 15 min; Wave Length: 220/254 nm; RT1(min): 8.19; RT2(min): 12.94; |
| 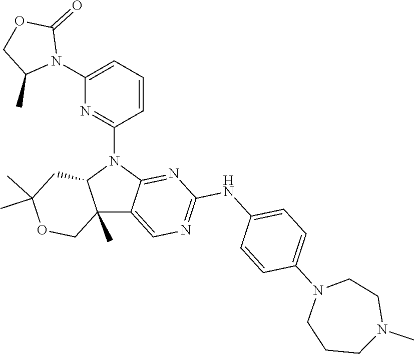 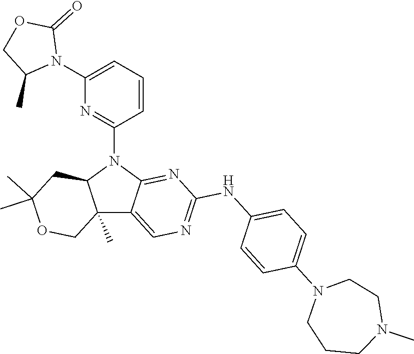 | 298, 299 | 1H NMR (400 MHz, DMSO-d6) δ 8.96 (s, 1H), 8.41 (d, J = 8.0 Hz, 1H), 7.96 (s, 1H), 7.78 (t, J = 8.4 Hz, 1H), 7.64 (d, J = 8.0 Hz, 1H), 7.43-7.41 (m, 2H), 6.66- 6.64 (m, 2H), 4.79-4.78 (m, 1H), 4.56 (t, J = 8.4 Hz, 1H), 4.48-4.44 (m, 1H), 4.11-4.08 (m, 1H), 3.80-3.77 (m, 1H), 3.68-3.65 (m, 1H), 3.50-3.40 (m, 4H), 2.67-2.59 (m, 2H), 2.48-2.41 (m, 2H), 2.36-2.32 (m, 1H), 2.26 (s, 3H), 1.90- 1.88 (m, 2H), 1.59-1.56 (m, 1H), 1.47 (d, J = 6.0 Hz, 3H), 1.27 (s, 3H), 1.25 (s, 3H), 1.07 (s, 3H). 1H NMR (400 MHz, DMSO-d6) δ 8.96 (s, 1H), 8.42 (d, J = 8.0 Hz, 1H), 7.98 (s, 1H), 7.76 (t, J = 8.4 Hz, 1H), 7.62 (d, J = 7.6 Hz, 1H), 7.43-7.41 (m, 2H), 6.66- 6.64 (m, 2H), 4.94-4.91 (m, 1H), 4.66- 4.62 (m, 1H), 4.53 (t, J = 8.0 Hz, 1H), 4.13-4.10 (m, 1H), 3.85-3.82 (m, 1H), 3.68-3.65 (m, 1H), 3.50-3.40 (m, 4H), 2.67-2.61 (m, 2H), 2.48-2.42 (m, 2H), 2.26 (s, 3H), 2.16-2.11 (m, 1H), 1.92- 1.88 (m, 2H), 1.55-1.51 (m, 1H), 1.37 (d, J = 6.0 Hz, 3H), 1.36 (s, 3H), 1.24 (s, 3H), 1.06 (s, 3H). | LCMS (ES, m/z): 599 [M + H]+, Rt 0.758 min LCMS (ES, m/z): 599 [M + H]+, Rt 0.759 min. | Column: CHIRAL ART Amylose-SA, 2*25 cm, 5 μm; Mobile Phase A: MtBE(0.5% 2M NH3- MeOH)-HPLC, Mobile Phase B: EtOH-HPLC; Flow rate: 20 mL/min; Gradient: 50% B to 50% B in 31 min; Wave Length: 220/254 nm; RT1(min): 8.75; RT2(min): 18.741; |
| 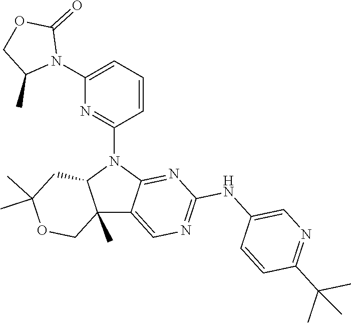 [SMILES: C[C@H]1COC(=O)N1c1cccc(N2c3nc(Nc4ccc(C(C)(C)C)nc4)ncc3[C@]3(C)COC(C)(C)C[C@H]23)n1] 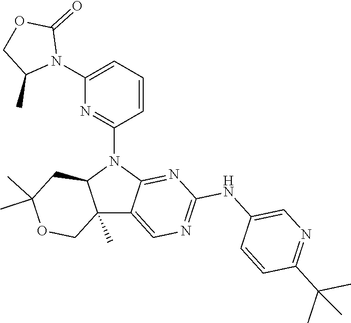 [SMILES: C[C@H]1COC(=O)N1c1cccc(N2c3nc(Nc4ccc(C(C)(C)C)nc4)ncc3[C@@]3(C)COC(C)(C)C[C@@H]23)n1] | 300, 301 | 1H NMR (300 MHz, DMSO-d6) δ 9.47 (s, 1H), 8.76 (d, J = 2.4 Hz, 1H), 8.42 (d, J = 8.1 Hz, 1H), 8.14-8.10 (m, 1H), 8.07 (s, 1H), 7.83 (t, J = 8.1 Hz, 1H), 7.69 (d, J = 8.1 Hz, 1H), 7.37 (d, J = 8.7 Hz, 1H), 4.81-4.78 (m, 1H), 4.60-4.47 (m, 2H), 4.13-4.09 (m, 1H), 3.85-3.81 (m, 1H), 3.71-3.67 (m, 1H), 2.39-2.33 (m, 1H), 1.64-1.58 (m, 1H), 1.49 (d, J = 6.0 Hz, 3H), 1.31 (s, 9H), 1.27 (s, 3H), 1.26 (s, 3H), 1.08 (s, 3H). 1H NMR (300 MHz, DMSO-d6) δ 9.47 (s, 1H), 8.76 (d, J = 2.4 Hz, 1H), 8.41 (d, J = 8.1 Hz, 1H), 8.14-8.10 (m, 1H), 8.08 (s, 1H), 7.83 (t, J = 8.1 Hz, 1H), 7.68 (d, J = 8.1 Hz, 1H), 7.37 (d, J = 8.7 Hz, 1H), 4.96-4.92 (m, 1H), 4.71-4.67 (m, 1H), 4.57-4.52 (m, 1H), 4.15-4.11 (m, 1H), 3.89-3.85 (m, 1H), 3.72-3.68 (m, 1H), 2.18-2.12 (m, 1H), 1.59-1.52 (m, 1H), 1.39 (d, J = 6.0 Hz, 3H), 1.31 (s, 9H), 1.27 (s, 3H), 1.25 (s, 3H), 1.07 (s, 3H). | LCMS (ES, m/z): 544 [M + H]+. Rt 0.829 min. LCMS (ES, m/z): 544 [M + H]+. Rt 0.823 min. | Column: CHIRAL ART Amylose-SA, 2*25 cm, 5 μm; Mobile Phase A: Hex (0.5% 2M NH3-MeOH)- HPLC, Mobile Phase B: EtOH- HPLC; Flow rate: 20 mL/min; Gradient: 30% B to 30% B in 18 min; Wave Length: 220/254 nm; RT1(min): 11.158; RT2(min): 13.925 |
| 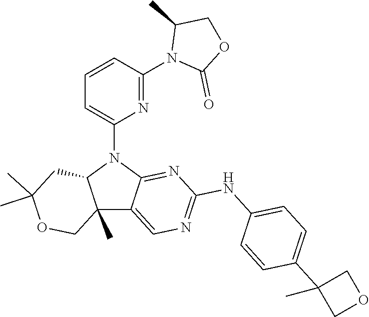 [SMILES: C[C@H]1COC(=O)N1c1cccc(N2c3nc(Nc4ccc(C5(C)COC5)cc4)ncc3[C@]3(C)COC(C)(C)C[C@H]23)n1] 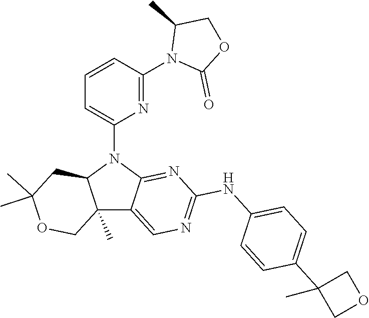 [SMILES: C[C@H]1COC(=O)N1c1cccc(N2c3nc(Nc4ccc(C5(C)COC5)cc4)ncc3[C@@]3(C)COC(C)(C)C[C@@H]23)n1] | 302, 303 | 1H NMR (300 MHz, DMSO-d6) δ 9.39 (s, 1H), 8.44 (d, J = 8.1 Hz, 1H), 8.07 (s, 1H), 7.85 (t, J = 8.1 Hz, 1H), 7.74-7.66 (m, 3H), 7.21 (d, J = 8.7 Hz, 2H), 4.83- 4.73 (m, 3H), 4.61-4.48 (m, 4H), 4.13- 4.09 (m, 1H), 3.85-3.81 (m, 1H), 3.71- 3.67 (m, 1H), 2.39-2.33 (m, 1H), 1.64- 1.56 (m, 4H), 1.50 (d, J = 6.0 Hz, 3H), 1.30 (s, 3H), 1.26 (s, 3H) 1.08 (s, 3H). 1H NMR (300 MHz, DMSO-d6) δ 9.39 (s, 1H), 8.45 (d, J = 8.4 Hz, 1H), 8.08 (s, 1H), 7.86 (t, J = 8.1 Hz, 1H), 7.74-7.65 (m, 3H), 7.21 (d, J = 8.4 Hz, 2H), 4.97- 4.91 (m, 1H), 4.83 (d, J = 5.7 Hz, 2H), 4.71-4.66 (m, 1H), 4.58-4.53 (m, 3H), 4.15-4.09 (m, 1H), 3.89-3.85 (m, 1H), 3.72-3.68 (m, 1H), 2.19-2.11 (m, 1H), 1.64-1.52 (m, 4H), 1.40 (d, J = 6.0 Hz, 3H), 1.27 (s, 3H), 1.25 (s, 3H), 1.07 (s, 3H). | LCMS (ES, m/z): 557 [M + H]+, Rt 0.834 min. LCMS (ES) m/z): 557 [M + H]+, Rt 0.835 min. | Column: CHIRAL ART Amylose-SA, 2*25 cm, 5 μm; Mobile Phase A: MtBE(0.5% 2M NH3- MeOH)-HPLC, Mobile Phase B: EtOH-HPLC; Flow rate: 20 mL/min; Gradient: 30% B to 30% B in 18 min; Wave Length: 220/254 nm; RT1(min): 7.33; RT2(min): 13.44; Sample Solvent: EtOH- HPLC; |
| 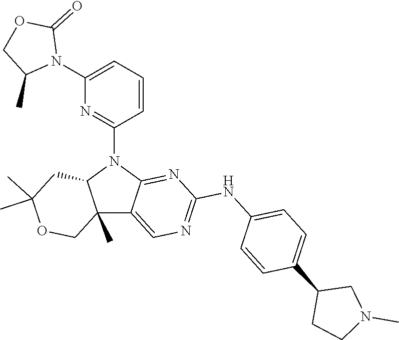 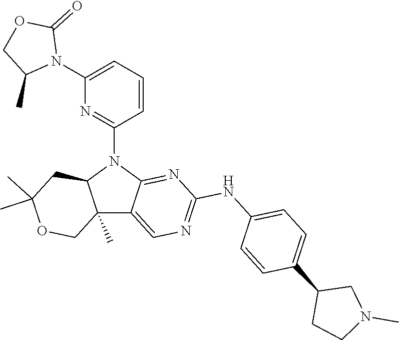 | 304, 305 | 1H NMR (300 MHz, DMSO-d6) δ 9.32 (s, 1H), 8.43 (d, J = 8.1 Hz, 1H), 8.05 (s, 1H), 7.83 (t, J = 8.1 Hz, 1H), 7.72-7.60 (m, 3H), 7.20 (d, J = 8.4 Hz, 2H), 4.86- 4.74 (m, 1H), 4.63-4.45 (m, 2H), 4.16- 4.04 (m, 1H), 3.88-3.64 (m, 2H), 3.30- 3.20 (m, 1H), 2.87 (t, J = 8.4 Hz, 1H), 2.72-2.54 (m, 2H), 2.42-2.15 (m, 6H), 1.85-1.67 (m, 1H), 1.67-1.55 (m, 1H), 1.49 (d, J = 6.3 Hz, 3H), 1.30 (s, 3H), 1.26 (s, 3H), 1.08 (s, 3H). 1H NMR (300 MHz, DMSO-d6) δ 9.32 (s, 1H), 8.43 (d, J = 8.1 Hz, 1H), 8.07 (s, 1H), 7.84 (t, J = 8.1 Hz, 1H), 7.70-7.60 (m, 3H), 7.19 (d, J = 8.4 Hz, 2H), 5.00- 4.87 (m, 1H), 4.73-4.64 (m, 1H), 4.60- 4.50 (m, 1H), 4.17-4.07(m, 1H), 3.90- 3.64 (m, 2H), 3.32-3.20 (m, 1H), 2.87 (t, J = 8.4 Hz, 1H), 2.72-2.54 (m, 2H), 2.42-2.10 (m, 6H), 1.84-1.67 (m, 1H), 1.67-1.55 (m, 1H), 1.39 (d, J = 6.3 Hz, 3H), 1.27 (s, 3H), 1.25 (s, 3H), 1.07 (s, 3H). | LCMS (ES, m/z): 570 [M + H]+, Rt 1.418 min. LCMS (ES, m/z): 570 [M + H]+, Rt 0.675 min. | Column: CHIRALPAK IG, 2 × 25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 30% B to 30% B in 16.5 min; Wave Length: 220/254 nm; RT1(min): 12.39; RT2(min): 15.10; |
| 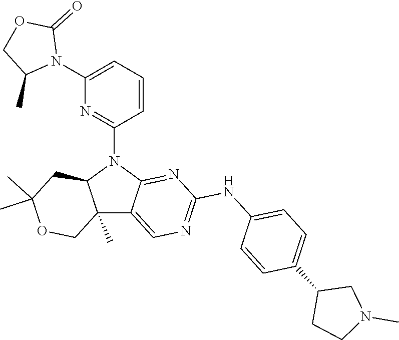 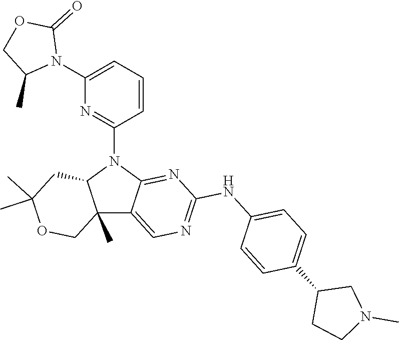 | 306, 307 | 1H NMR (300 MHz, DMSO-d6) δ 9.32 (s, 1H), 8.43 (d, J = 7.8 Hz, 1H), 8.06 (s, 1H), 7.83 (t, J = 7.8 Hz, 1H), 7.69-7.63 (m, 3H), 7.19 (d, J = 8.4 Hz, 2H), 4.86- 4.74 (m, 1H), 4.63-4.45 (m, 2H), 4.18- 4.07 (m, 1H), 3.84-3.80 (m, 1H), 3.71- 3.67 (m, 1H), 3.30-3.24 (m, 1H), 2.87 (t, J = 8.1 Hz, 1H), 2.67-2.58 (m, 2H), 2.40-2.35 (m, 2H), 2.30 (s, 3H), 2.25- 2.07 (m, 1H), 1.84-1.67 (m, 1H), 1.65- 1.52 (m, 1H), 1.50 (d, J = 6.0 Hz, 3H), 1.27 (s, 3H), 1.25 (s, 3H), 1.07 (s, 3H). 1H NMR (300 MHz, DMSO-d6) δ 9.32 (s, 1H), 8.43 (d, J = 8.1 Hz, 1H), 8.05 (s, 1H), 7.83 (t, J = 8.1 Hz, 1H), 7.69-7.63 (m, 3H), 7.20 (d, J = 8.4 Hz, 2H), 4.87- 4.72 (m, 1H), 4.62-4.46 (m, 2H), 4.13- 4.10 (m, 1H), 3.87-3.81 (m, 1H), 3.71- 3.64 (m, 1H), 3.33-3.26 (m, 1H), 2.87 (t, J = 8.4 Hz, 1H), 2.65-2.59 (m, 2H), 2.41-2.31 (m, 6H), 1.85-1.67 (m, 1H), 1.66-1.53 (m, 1H), 1.49 (d, J = 6.0 Hz, 3H), 1.30 (s, 3H), 1.26 (s, 3H), 1.08 (s, 3H). | LCMS (ES, m/z): 570 [M + H]+, Rt 1.261 min. LCMS (ES) m/z): 570 [M + H]+, Rt 2.981 min. | Column: CHIRALPAK IG, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 20% B to 20% B in 18 min; Wave Length: 220/254 nm; RT1(min): 13.82; RT2(min): 16.89; |
| 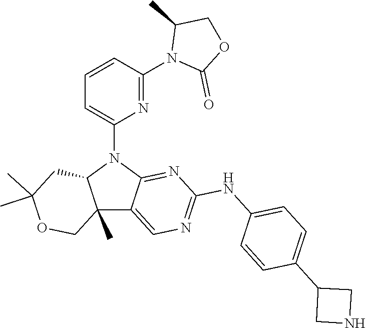 [SMILES: C[C@H]1COC(=O)N1c1cccc(N2c3nc(Nc4ccc(C5CNC5)cc4)ncc3[C@]3(C)COC(C)(C)C[C@H]23)n1] 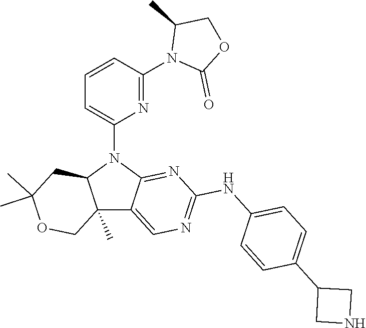 [SMILES: C[C@H]1COC(=O)N1c1cccc(N2c3nc(Nc4ccc(C5CNC5)cc4)ncc3[C@@]3(C)COC(C)(C)C[C@@H]23)n1] | 308, 310 | 1H NMR (300 MHz, DMSO-d6) δ 9.37 (s, 1H), 8.43 (d, J = 8.1 Hz, 1H), 8.07 (s, 1H), 7.84 (t, J = 8.4 Hz, 1H), 7.71-7.66 (m, 3H), 7.28 (d, J = 8.7 Hz, 2H), 4.83- 4.77 (m, 1H), 4.61-4.48 (m, 2H), 4.13- 4.09 (m, 1H), 3.85-3.71 (m, 5H), 3.67- 3.61 (m, 2H), 2.39-2.33 (m, 1H), 1.64- 1.56 (m, 1H), 1.49 (d, J = 6.3 Hz, 3H), 1.30 (s, 3H), 1.26 (s, 3H), 1.08 (s, 3H). 1H NMR (300 MHz, DMSO-d6) δ 9.37 (s, 1H), 8.43 (d, J = 8.1 Hz, 1H), 8.08 (s, 1H), 7.85 (t, J = 8.4 Hz, 1H), 7.68 (t, J = 9.0 Hz, 3H), 7.28 (d, J = 8.4 Hz, 2H), 4.97-4.91 (m, 1H), 4.71-4.66 (m, 1H), 4.55 (t, J = 8.1 Hz, 1H), 4.15-4.11 (m, 1H), 3.89-3.60 (m, 7H), 2.19-2.13 (m, 1H), 1.60-1.52 (m, 1H), 1.39 (d, J = 6.3 Hz, 3H), 1.27 (s, 3H), 1.25 (s, 3H), 1.07 (s, 3H). | LCMS (ES) m/z): 542 [M + H]+, Rt 0.671 min. LCMS (ES) m/z): 542 [M + H]+, Rt 0.675 min. | Column: CHIRALPAK IG, 2 × 25 cm, 5 μm; Mobile Phase A: Hex: DCM = 3:1 (0.5% 2M NH3-MeOH)- HPLC, Mobile Phase B: EtOH- HPLC(30% to 30% in 18 min); Flow rate: 20 mL/min; Wave Length: 220/254 nm; RT1(min): 12.48; RT2(min): 16.20 |
| 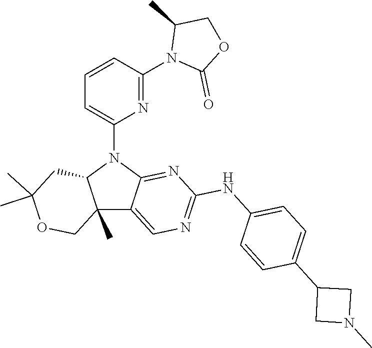 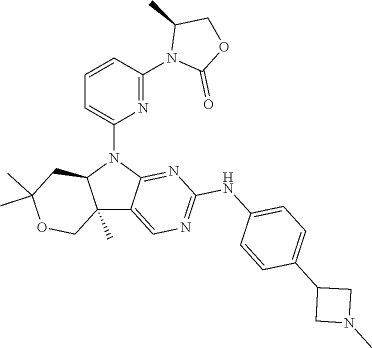 | 309, 311 | 1H NMR (300 MHz, DMSO-d6) δ 9.36 (s, 1H), 8.44 (d, J = 8.4 Hz, 1H), 8.06 (s, 1H), 7.84 (t, J = 8.4 Hz, 1H), 7.70-7.67 (m, 3H), 7.26 (d, J = 8.4 Hz, 2H), 4.83- 4.77 (m, 1H), 4.61-4.48 (m, 2H), 4.13- 4.09 (m, 1H), 3.85-3.81 (m, 1H), 3.71- 3.67 (m, 1H), 3.63-3.49 (m, 3H), 3.06- 3.02 (m, 2H), 2.39-2.33 (m, 1H), 2.27 (s, 3H), 1.64-1.56 (m, 1H), 1.49 (d, J = 6.3 Hz, 3H), 1.30 (s, 3H), 1.27 (s, 3H), 1.08 (s, 3H). 1H NMR (300 MHz, DMSO-d6) δ 9.36 (s, 1H), 8.43 (d, J = 8.1 Hz, 1H), 8.08 (s, 1H), 7.85 (t, J = 8.1 Hz, 1H), 7.70-7.65 (m, 3H), 7.26 (d, J = 8.7 Hz, 2H), 4.96- 4.92 (m, 1H), 4.71-4.66 (m, 1H), 4.55 (t, J = 8.4 Hz, 1H), 4.15-4.11 (m, 1H), 3.89-3.85 (m, 1H), 3.72-3.68 (m, 1H), 3.63-3.49 (m, 3H), 3.06-3.02 (m, 2H), 2.27 (s, 3H), 2.19-2.13 (m, 1H), 1.60- 1.52 (m, 1H), 1.39 (d, J = 6.0 Hz, 3H), 1.27 (s, 3H), 1.25 (s, 3H), 1.07 (s, 3H). | LCMS (ES, m/z): 556 [M + H]+, Rt 0.647 min. LCMS (ES, m/z): 556 [M + H]+, Rt 0.656 min. | Column: CHIRALPAK IG, 2 × 25 cm, 5 μm; Mobile Phase A: Hex: DCM = 3:1 (0.5% 2M NH3-MeOH)- HPLC, Mobile Phase B: EtOH- HPLC(30% to 30% in 18 min); Flow rate: 20 mL/min; Wave Length: 220/254 nm; RT1(min): 12.48; RT2(min): 16.20 |
| 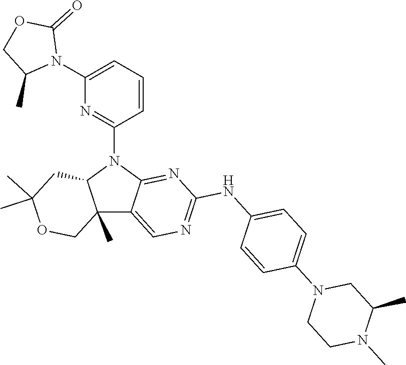 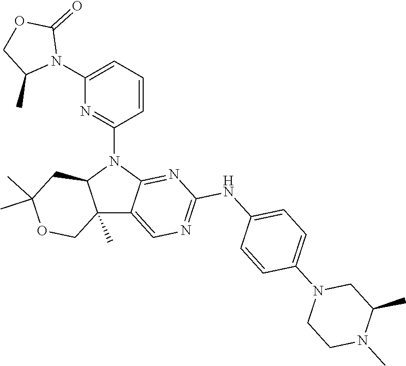 | 312, 313 | 1H NMR (300 MHz, DMSO-d6) δ 9.14 (s, 1H), 8.43 (d, J = 8.1 Hz, 1H), 8.01 (s, 1H), 7.83 (t, J = 8.1 Hz, 1H), 7.67 (d, J = 8.1 Hz, 1H), 7.56 (d, J = 9.0 Hz, 2H), 6.92 (d, J = 9.0 Hz, 2H), 4.82-4.76 (m, 1H), 4.60-4.46 (m, 2H), 4.13-4.09 (m, 1H), 3.83-3.79 (m, 1H), 3.70-3.66 (m, 1H), 3.46-3.45 (m, 2H), 2.85-2.74 (m, 1H), 2.72-2.68 (m, 1H), 2.72-2.17 (m, 7H), 1.58-1.50 (m, 1H), 1.39 (d, J = 6.3 Hz, 3H), 1.26 (s, 3H), 1.25 (s, 3H), 1.07 (s, 3H), 1.05 (s, 3H). 1H NMR (300 MHz, DMSO-d6) δ 9.13 (s, 1H), 8.43 (d, J = 8.4 Hz, 1H), 8.02 (s, 1H), 7.83 (t, J = 8.1 Hz, 1H), 7.66 (d, J = 8.1 Hz, 1H), 7.56 (d, J = 9.0 Hz, 2H), 6.91 (d, J = 9.0 Hz, 2H), 4.88-4.93 (m, 1H), 4.67-4.64 (m, 1H), 4.57-4.52 (m, 1H), 4.14-4.10 (m, 1H), 3.87-3.83 (m, 1H), 3.70-3.66 (m, 1H), 3.47-3.44 (m, 2H), 2.83-2.80 (m, 1H), 2.69-2.68 (m, 1H), 2.37-2.26 (m, 2H), 2.22 (s, 3H), 2.18-2.12 (m, 2H), 1.54-1.53 (m, 1H), 1.39 (d, J = 6.0 Hz, 3H), 1.26 (s, 3H), 1.25 (s, 3H), 1.07 (s, 3H), 1.05 (s, 3H). | LCMS (ES, m/z): 599 [M + H]+. Rt 0.651 min. LCMS (ES, m/z): 599 [M + H]+. Rt 0.652 min. | Column: CHIRAL ART Amylose-SA, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 20% B to 20% B in 13 min; Wave Length: 220/254 nm; RT1(min): 6.08; RT2(min): 11.02 |
| 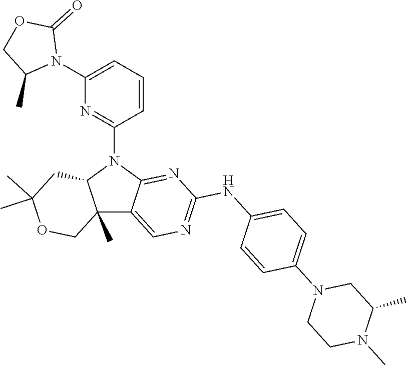 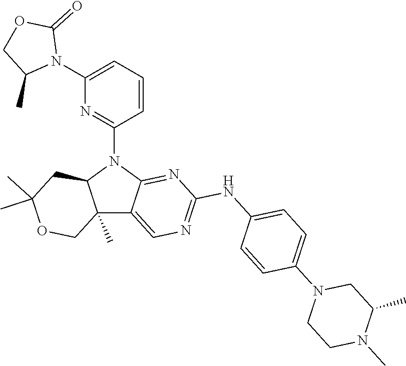 | 314, 315 | 1H NMR (300 MHz, DMSO-d6) δ 9.14 (s, 1H), 8.42 (d, J = 8.1 Hz, 1H), 8.01 (s, 1H), 7.82 (t, J = 8.1 Hz, 1H), 7.66 (d, J = 8.1 Hz, 1H), 7.59-7.50 (m, 2H), 6.95- 6.84 (m, 2H), 4.85-4.71 (m, 1H), 4.63- 4.41 (m, 2H), 4.15-3.98 (m, 1H), 3.87- 3.63 (m, 2H), 3.51-3.39 (m, 2H), 2.91- 2.61 (m, 2H), 2.41-2.09 (m, 7H), 1.66- 1.42 (m, 4H), 1.29 (s, 3H), 1.24 (s, 3H), 1.06 (s, 6H). 1H NMR (300 MHz, DMSO-d6) δ 9.14 (s, 1H), 8.42 (d, J = 8.1 Hz, 1H), 8.02 (s, 1H), 7.83 (t, J = 8.1 Hz, 1H), 7.64 (d, J = 8.1 Hz, 1H), 7.55 (d, J = 8.7 Hz, 2H), 6.95-6.84 (m, 2H), 5.00-4.82 (m, 1H), 4.68-4.65 (m, 1H), 4.54 (t, J = 8.1 Hz, 1H), 4.15-3.98 (m, 1H), 3.90-3.63 (m, 2H), 3.45-3.38 (m, 2H), 2.89-2.63 (m, 2H), 2.41-2.09 (m, 7H), 1.55-1.50 (m, 1H), 1.38 (d, J = 6.3 Hz, 3H), 1.27 (s, 3H), 1.25 (s, 3H), 1.09-0.98 (m, 6H). | LCMS (ES) m/z): 599 [M + H] +; RT: 0.650 min. LCMS (ES, m/z): 599 [M + H] +; RT: 1.328 min. | Column: CHIRAL ART Amylose-SA, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 10% B to 10% B in 12 min; Wave Length: 220/254 nm; RT1(min): 7.36; RT2(min): 9.69; |
| 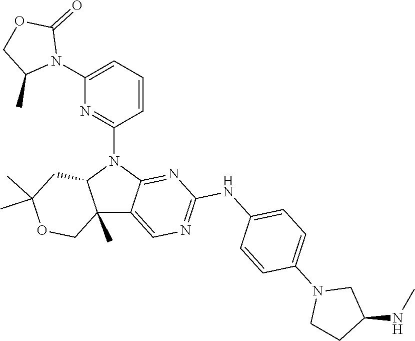 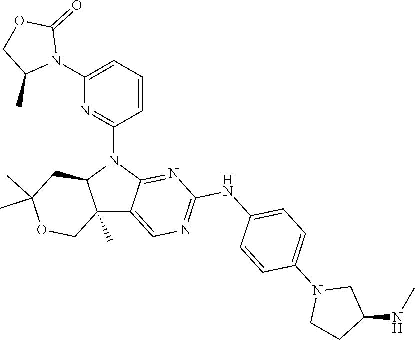 | 316, 317 | 1H NMR (400 MHz, DMSO-d6) δ 8.98 (s, 1H), 8.45 (d, J = 8.4 Hz, 1H), 7.96 (s, 1H), 7.78 (t, J = 8.1 Hz, 1H), 7.63 (d, J = 8.1 Hz, 1H), 7.43 (d, J = 8.4 Hz, 2H), 6.50 (d, J = 8.7 Hz, 2H), 4.85-4.70 (m, 1H), 4.65-4.56 (m, 2H), 4.11-4.08 (m, 1H), 3.80-3.77 (m, 1H), 3.67-3.64 (m, 1H), 3.45-3.37 (m, 1H), 3.33-3.21 (m, 3H), 3.03-2.97 (m, 1H), 2.40-2.30 (m, 4H), 2.20-2.09 (m, 1H), 1.88-1.85 (m, 1H), 1.80-1.78 (m, 1H), 1.49 (d, J = 6.0 Hz, 3H), 1.29 (s, 3H), 1.26 (s, 3H), 1.06 (s, 3H). 1H NMR (400 MHz, DMSO-d6) δ 9.05 (s, 1H), 8.42 (d, J = 8.1 Hz, 1H), 8.01 (s, 1H), 7.81 (t, J = 8.1 Hz, 1H), 7.64 (d, J = 7.8 Hz, 1H), 7.52 (d, J = 8.4 Hz, 2H), 6.60 (d, J = 9.0 Hz, 2H), 4.95-4.85 (m, 1H), 4.70-4.60 (m, 1H), 4.58-4.52 (m, 1H), 4.15-4.10 (m, 1H), 3.85-3.80 (m, 1H), 3.78-3.61 (m, 2H), 3.52-3.41 (m, 2H), 3.33-3.27 (m, 2H), 2.58 (s, 3H), 2.36-2.29 (m, 1H), 2.19-1.97 (m, 2H), 1.59-1.51 (m, 1H), 1.39 (d, J = 6.3 Hz, 3H), 1.25 (s, 6H), 1.07 (s, 3H). | LCMS (ES, m/z): 585 [M + H]+, Rt 0.685 min LCMS (ES, m/z): 585 [M + H]+, Rt 0.695 min | Column: CHIRALPAK IH- 3, 3.0*50 mm, 3 μm; Mobile Phase B: MeOH(1%-2M- NH3-MeOH); Flow rate: 2 mL/min; Gradient: isocratic 10% B; Wave Length: 220 nm |
| 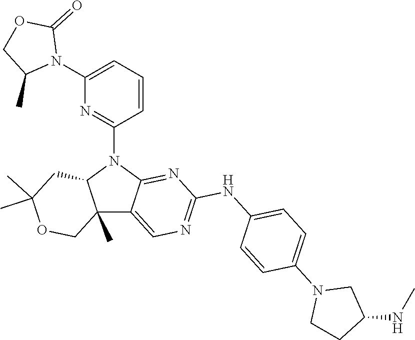 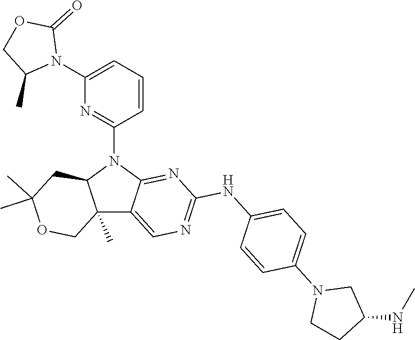 | 318, 319 | 1H NMR (300 MHz, DMSO-d6) δ 8.97 (s, 1H), 8.44 (d, J = 8.4 Hz, 1H), 7.98 (s, 1H), 7.80 (t, J = 8.1 Hz, 1H), 7.66 (d, J = 8.1 Hz, 1H), 7.47 (d, J = 8.7 Hz, 2H), 6.52 (d, J = 8.7 Hz, 2H), 4.83-4.78 (m, 1H), 4.57 (t, J = 8.4 Hz, 1H), 4.50-4.45 (m, 1H), 4.13-4.09 (m, 1H), 3.82-3.78 (m, 1H), 3.69-3.65 (m, 1H), 3.45-3.39 (m, 1H), 3.34-3.21 (m, 3H), 2.30-2.95 (m, 1H), 2.38-2.32 (m, 4H), 2.14-2.05 (m, 2H), 1.82-1.76 (m, 1H), 1.62-1.48 (m, 4H), 1.29 (s, 3H), 1.26 (s, 3H), 1.08 (s, 3H). 1H NMR (300 MHz, DMSO-d6) δ 8.97 (s, 1H), 8.44 (d, J = 8.1 Hz, 1H), 7.99 (s, 1H), 7.80 (t, J = 8.1 Hz, 1H), 7.65 (d, J = 7.8 Hz, 1H), 7.47 (d, J = 9.0 Hz, 2H), 6.52 (d, J = 8.7 Hz, 2H), 4.97-4.92 (m, 1H), 4.68-4.63 (m, 1H), 4.55 (t, J = 8.4 Hz, 1H), 4.15-4.11 (m, 1H), 3.87-3.83 (m, 1H), 3.70-3.66 (m, 1H), 3.45-3.39 (m, 1H), 3.34-3.81 (m, 3H), 2.30-2.95 (m, 1H), 2.33 (s, 3H), 2.18-2.08 (m, 3H), 1.84-1.75 (m, 1H), 1.57-1.50 (m, 1H), 1.40 (d, J = 6.3 Hz, 3H), 1.25 (s, 6H), 1.07 (s, 3H). | LCMS (ES, m/z): 585 [M + H]+. Rt 0.660 min LCMS (ES, m/z): 585 [M + H]+. Rt 0.663 min | Column: CHIRALPAK IA, 2*25 cm, 20 μm; Mobile Phase A: Hex(0.5% 2M NH3-MeOH)- HPLC, Mobile Phase B: EtOH- HPLC; Flow rate: 20 mL/min; Gradient: 35% B to 35% B in 30 min; Wave Length: 220/254 nm; RT1(min): 13.687; RT2(min): 21.458; Sample Solvent: EtOH- HPLC; |
| 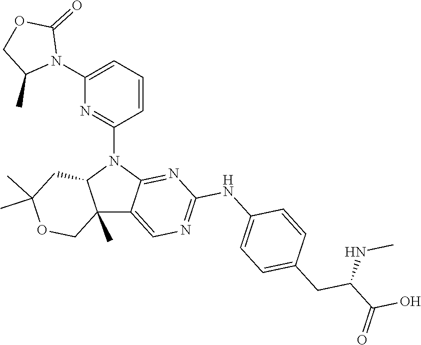 [SMILES: CN[C@@H](Cc1ccc(Nc2ncc3c(n2)N(c2cccc(N4C(=O)OC[C@@H]4C)n2)[C@H]2CC(C)(C)OC[C@@]32C)cc1)C(=O)O] 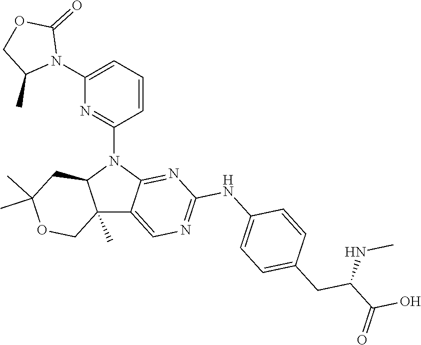 [SMILES: CN[C@@H](Cc1ccc(Nc2ncc3c(n2)N(c2cccc(N4C(=O)OC[C@@H]4C)n2)[C@@H]2CC(C)(C)OC[C@]32C)cc1)C(=O)O] | 320, 321 | 1H NMR (300 MHz, DMSO-d6) δ 9.36 (s, 1H), 8.44 (d, J = 8.4 Hz, 1H), 8.05 (s, 1H), 7.86 (t, J = 8.1 Hz, 1H), 7.68-7.63 (m, 3H), 7.20 (d, J = 8.7 Hz, 2H), 4.82- 4.76 (m, 1H), 4.60-4.47 (m, 2H), 4.13- 4.09 (m, 1H), 3.84-3.80 (m, 1H), 3.70- 3.66 (m, 1H), 3.34 (s, 1H), 3.09-3.03 (m, 1H), 2.95-2.90 (m, 1H), 2.50-2.32 (m, 4H), 1.64-1.61 (m, 1H), 1.49 (d, J = 6.0 Hz, 3H), 1.32 (s, 3H), 1.26 (s, 3H), 1.07 (s, 3H). 1H NMR (300 MHz, DMSO-d6) δ 9.36 (s, 1H), 8.44 (d, J = 8.4 Hz, 1H), 8.07 (s, 1H), 7.87 (d, J = 8.1 Hz, 1H), 7.67-7.62 (m, 3H), 7.19 (d, J = 8.7 Hz, 2H), 4.95- 4.91 (m, 1H), 4.71-4.66 (m, 1H), 4.57- 4.52 (m, 1H), 4.15-4.11 (m, 1H), 3.88- 3.84 (m, 1H), 3.71-3.67 (m, 1H), 3.34 (s, 1H), 3.09-3.02 (m, 1H), 2.94-2.89 (m, 1H), 2.51 (s, 3H), 2.35-2.28 (m, 1H), 1.61-1.59 (m, 1H), 1.39 (d, J = 6.0 Hz, 3H), 1.27 (s, 3H), 1.25 (s, 3H), 1.06 (s, 3H). | LCMS (ES, m/z): 588 [M + H]+. Rt 0.631 min. LCMS (ES, m/z): 588 [M + H]+. Rt 0.629 min. | Column: CHIRAL ART Cellulose-SC, 2*25 cm, 5 μm; Mobile Phase A: Hex(0.5% 2M NH3-MeOH)- HPLC, Mobile Phase B: EtOH- HPLC; Flow rate: 20 mL/min; Gradient: 40% B to 40% B in 13 min; Wave Length: 220/254 nm; RT1(min): 10.07; RT2(min): 12.23; |
| 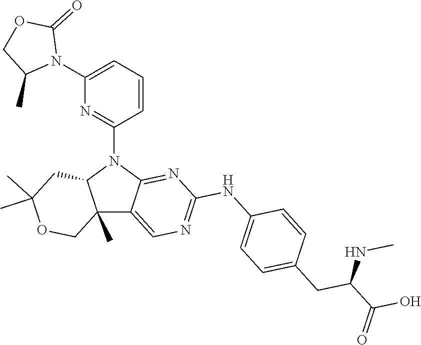 [SMILES: CN[C@H](Cc1ccc(Nc2ncc3c(n2)N(c2cccc(N4C(=O)OC[C@@H]4C)n2)[C@H]2CC(C)(C)OC[C@@]32C)cc1)C(=O)O] 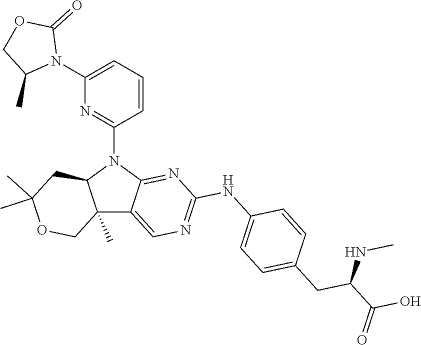 [SMILES: CN[C@H](Cc1ccc(Nc2ncc3c(n2)N(c2cccc(N4C(=O)OC[C@@H]4C)n2)[C@@H]2CC(C)(C)OC[C@]32C)cc1)C(=O)O] | 322, 323 | 1H NMR (300 MHz, DMSO-d6) δ 9.37 (s, 1H), 8.44 (d, J = 8.1 Hz, 1H), 8.06 (s, 1H), 7.89 (t, J = 8.1 Hz, 1H), 7.69-7.63 (m, 3H), 7.21 (d, J = 8.4 Hz, 2H), 4.83- 4.80 (m, 1H), 4.61-4.47 (m, 2H), 4.13- 4.09 (m, 1H), 3.84-3.80 (m, 1H), 3.71- 3.67 (m, 1H), 3.33-3.28 (m, 1H), 3.10- 3.03 (m, 1H), 2.95-2.88 (m, 1H), 2.37- 2.33 (m, 4H), 1.64-1.56 (m, 1H), 1.50 (d, J = 6.0 Hz, 3H), 1.30 (s, 3H), 1.26 (s, 3H), 1.08 (s, 3H). 1H NMR (300 MHz, DMSO-d6) δ 9.37 (s, 1H), 8.44 (d, J = 8.1 Hz, 1H), 8.07 (s, 1H), 7.89 (t, J = 8.1 Hz, 1H), 7.67-7.63 (m, 3H), 7.21 (d, J = 8.4 Hz, 2H), 4.97- 4.91 (m, 1H), 4.71-4.66 (m, 1H), 4.55 (d, J = 8.4 Hz, 1H), 4.15-4.11 (m, 1H), 3.88-3.84 (m, 1H), 3.71-3.67 (m, 1H), 3.27-3.18 (m, 1H), 3.10-3.03 (m, 1H), 2.95-2.88 (m, 1H), 2.36 (s, 3H), 2.19- 2.12 (m, 1H), 1.60-1.52 (m, 1H), 1.40 (d, J = 6.0 Hz, 3H), 1.27 (s, 3H), 1.25 (s, 3H), 1.07 (s, 3H). | LCMS (ES, m/z): 588 M + H]+. Rt 0.632 min LCMS (ES, m/z): 588 [M + H]+. Rt 0.632 min | Column: CHIRAL ART Cellulose-SC, 2*25 cm, 5 μm; Mobile Phase A: Hex (0.5% 2M NH3-MeOH)- HPLC, Mobile Phase B: EtOH- HPLC; Flow rate: 20 mL/min; Gradient: 40% B to 40% B in 14 min; Wave Length: 220/254 nm; RT1(min): 10.10; RT2(min): 12.40 |
| 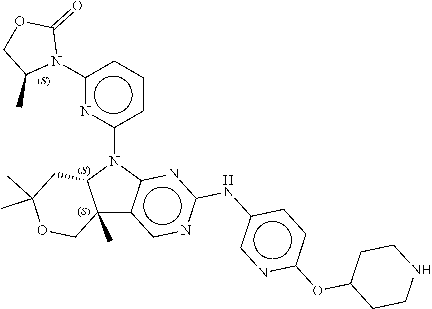 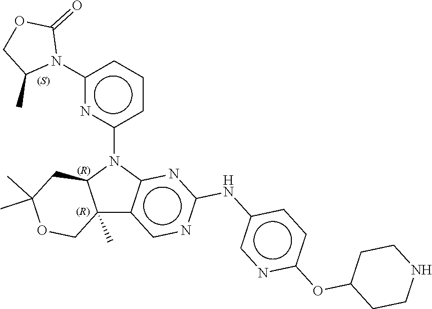 | 324, 325 | 1H NMR (300 MHz, DMSO-d6) δ 9.29 (s, 1H), 8.42-8.31 (m, 2H), 8.06-7.98 (m, 2H), 7.80 (t, J = 8.1 Hz, 1H), 7.66 (d, J = 8.1 Hz, 1H), 6.76 (d, J = 8.7 Hz, 1H), 5.05-4.90 (m, 1H), 4.85-4.72 (m, 1H), 4.63-4.44 (m, 2H), 4.15-4.08 (m, 1H), 3.86-3.76 (m, 1H), 3.73-3.63 (m, 1H), 2.99-2.90 (m, 2H), 2.67-2.56 (m, 1H), 2.38-2.31 (m, 2H), 2.01-1.90 (m, 2H), 1.65-1.41 (m, 6H), 1.28 (s, 3H), 1.25 (s, 3H), 1.07 (s, 3H). 1H NMR (300 MHz, DMSO-d6) δ 9.29 (s, 1H), 8.42-8.31 (m, 2H), 8.06-7.98 (m, 2H), 7.81 (t, J = 8.1 Hz, 1H), 7.65 (d, J = 8.1 Hz, 1H), 6.76 (d, J = 8.7 Hz, 1H), 5.04-4.87 (m, 2H), 4.70-4.62 (m, 1H), 4.59-4.50 (m, 1H), 4.19-4.11 (m, 1H), 3.90-3.80 (m, 1H), 3.74-3.63 (m, 1H), 3.05-2.92 (m, 2H), 2.69-2.52 (m, 2H), 2.19-2.11 (m, 1H), 2.02-1.90 (m, 2H), 1.60-1.42 (m, 3H), 1.38 (d, J = 6.3 Hz, 3H), 1.25 (s, 3H), 1.24 (s, 3H), 1.06 (s, 3H). | LCMS (ES, m/z): 587 [M + H] +; RT: 0.667 min. LCMS (ES, m/z): 587 [M + H] +; RT: 0.668 min. | Column: CHIRALPAK IE, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 15% B to 15% B in 25 min; Wave Length: 254/220 nm; RT1(min): 18.97; RT2(min): 22.37; |
| 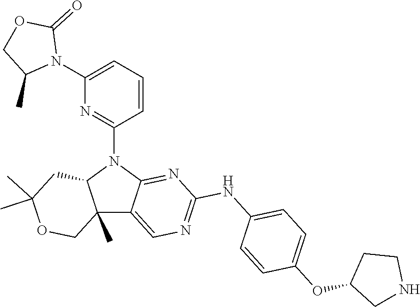 [SMILES: C[C@H]1COC(=O)N1c1cccc(N2c3nc(Nc4ccc(O[C@@H]5CCNC5)cc4)ncc3[C@]3(C)COC(C)(C)C[C@H]23)n1] 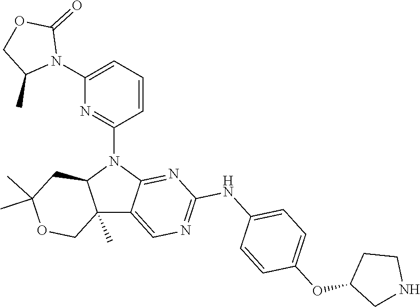 [SMILES: C[C@H]1COC(=O)N1c1cccc(N2c3nc(Nc4ccc(O[C@@H]5CCNC5)cc4)ncc3[C@@]3(C)COC(C)(C)C[C@@H]23)n1] | 326, 327 | 1H NMR (400 MHz, DMSO-d6) δ 9.19 (s, 1H), 8.40 (d, J = 8.4 Hz, 1H), 8.02 (s, 1H), 7.81 (t, J = 8.0 Hz, 1H), 7.67-7.58 (m, 3H), 6.85 (d, J = 9.2 Hz, 2H), 4.81- 4.78 (m, 2H), 4.57 (t, J = 8.4 Hz, 1H), 4.50-4.46 (m, 1H), 4.12-4.09 (m, 1H), 3.82-3.79 (m, 1H), 3.69-3.66 (m, 1H), 3.06-3.02 (m, 1H), 2.92-2.82 (m, 2H), 2.78-2.76 (m, 1H), 2.37-2.32 (m, 1H), 2.00-1.97 (m, 1H), 1.83-1.68 (m, 1H), 1.61-1.56 (m, 1H), 1.48 (d, J = 6.4 Hz, 3H), 1.29 (s, 3H), 1.26 (s, 3H), 1.07 (s, 3H). 1H NMR (400 MHz, DMSO-d6) δ 9.19 (s, 1H), 8.40 (d, J = 8.4 Hz, 1H), 8.03 (s, 1H), 7.82 (t, J = 8.0 Hz, 1H), 7.65-7.58 (m, 3H), 6.86 (d, J = 9.2 Hz, 2H), 4.95- 4.92 (m, 1H), 4.84-4.75 (m, 1H), 4.69- 4.65 (m, 1H), 4.54 (t, J = 8.4 Hz, 1H), 4.14-4.11 (m, 1H), 3.82-3.79 (m, 1H), 3.69-3.66 (m, 1H), 3.06-2.90 (m, 1H), 2.89-2.77 (m, 3H), 2.17-2.12 (m, 1H), 2.05-1.93 (m, 1H), 1.82-1.70 (m, 1H), 1.57-1.51 (m, 1H), 1.38 (d, J = 6.4 Hz, 3H), 1.29 (s, 3H), 1.26 (s, 3H), 1.06 (s, 3H) | LCMS (ES, m/z): 572 [M + H]+, Rt 0.672 min. LCMS (ES, m/z): 572 [M + H]+, Rt 0.670 min. | Column: Chiral ART Cellulose- SA, 2*25 cm, 5 μm; Mobile Phase A: Hex(0.5% 2M NH3-MeOH)- HPLC, Mobile Phase B: EtOH- HPLC; Flow rate: 20 mL/min; Gradient: 40% B to 40% B in 15 min; Wave Length: 254/220 nm; RT1(min): 9.23; RT2(min): 11.34; |
| 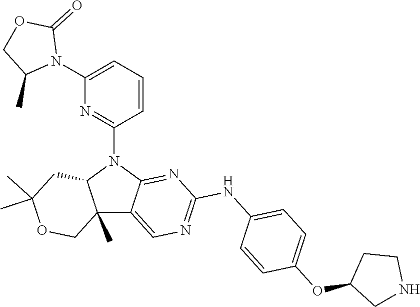 [SMILES: C[C@H]1COC(=O)N1c1cccc(N2c3nc(Nc4ccc(O[C@H]5CCNC5)cc4)ncc3[C@]3(C)COC(C)(C)C[C@H]23)n1] 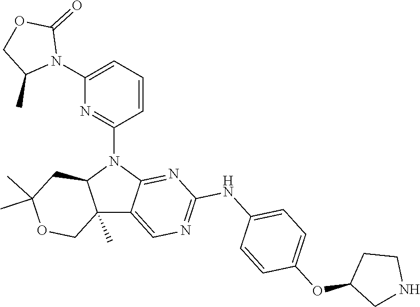 [SMILES: C[C@H]1COC(=O)N1c1cccc(N2c3nc(Nc4ccc(O[C@H]5CCNC5)cc4)ncc3[C@@]3(C)COC(C)(C)C[C@@H]23)n1] | 328, 329 | 1H NMR (400 MHz, DMSO-d6) δ 9.19 (s, 1H), 8.40 (d, J = 8.0 Hz, 1H), 8.02 (s, 1H), 7.82 (t, J = 8.0 Hz, 1H), 7.67-7.89 (m, 3H), 6.86 (d, J = 8.8 Hz, 2H), 4.83- 4.78 (m, 2H), 4.59-4.54 (m, 1H), 4.50- 4.46 (m, 1H), 4.12-4.09 (m, 1H), 3.82- 3.79 (m, 1H), 3.69-3.66 (m, 1H), 3.08- 3.04 (m, 1H), 2.94-2.76 (m, 3H), 2.37- 2.32 (m, 1H), 2.05-1.96 (m, 1H), 1.81- 1.74 (m, 1H), 1.61-1.56 (m, 1H), 1.48 (d, J = 6.4 Hz, 3H), 1.29 (s, 3H), 1.26 (s, 3H), 1.07 (s, 3H). 1H NMR (400 MHz, DMSO-d6) δ 9.20 (s, 1H), 8.40 (d, J = 8.0 Hz, 1H), 8.03 (s, 1H), 7.82 (t, J = 8.0 Hz, 1H), 7.66-7.58 (m, 3H), 6.86 (d, J = 9.2 Hz, 2H), 4.95- 4.91 (m, 1H), 4.83-4.80 (m, 1H), 4.69- 4.65 (m, 1H), 4.56-4.52 (m, 1H), 4.14- 4.11 (m, 1H), 3.86-3.83 (m, 1H), 3.70- 3.67 (m, 1H), 3.08-3.04 (m, 1H), 2.94- 2.79 (m, 3H), 2.17-2.12 (m, 1H), 2.05- 1.96 (m, 1H), 1.81-1.74 (m, 1H), 1.57- 1.51 (m, 1H), 1.38 (d, J = 6.4 Hz, 3H), 1.26 (s, 3H), 1.24 (s, 3H), 1.06 (s, 3H). | LCMS (ES, m/z): 572 [M + H]+, Rt 0.675 min. LCMS (ES) m/z): 572 [M + H]+, Rt 0.678 min. | Column: Chiral ART Cellulose- SA, 2 × 25 cm, 5 μm; Mobile Phase A: Hex(0.5% 2M NH3-MeOH)- HPLC, Mobile Phase B: EtOH- HPLC(50% to 50% in 15 min); Flow rate: 20 mL/min; Wave Length: 254/220 nm; RT1(min): 8.16; RT2(min): 10.93 |
| 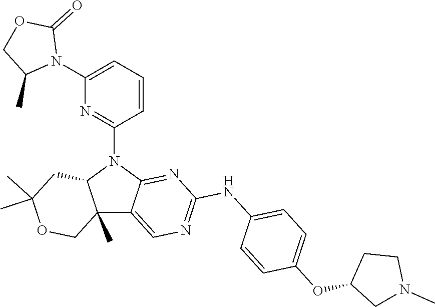 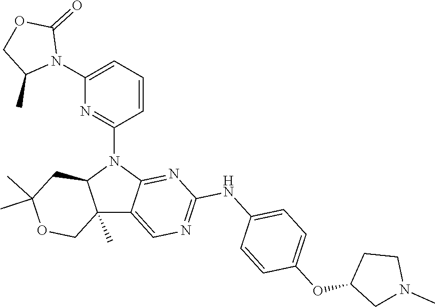 | 330, 331 | 1H NMR (400 MHz, DMSO-d6) δ 9.19 (s, 1H), 8.40 (d, J = 8.0 Hz, 1H), 8.01 (s, 1H), 7.82 (t, J = 8.0 Hz, 1H), 7.67-7.57 (m, 3H), 6.83 (d, J = 9.2 Hz, 2H), 4.81- 4.78 (m, 2H), 4.57 (t, J = 8.4 Hz, 1H), 4.50-4.46 (m, 1H), 4.12-4.09 (m, 1H), 3.82-3.79 (m, 1H), 3.69-3.66 (m, 1H), 2.80-2.76 (m, 1H), 2.65-2.62 (m, 1H), 2.60-2.57 (m, 1H), 2.38-2.32 (m, 2H), 2.26-2.2.25 (m, 4H), 1.82-1.71 (m, 1H), 1.63-1.53 (m, 1H), 1.48 (d, J = 6.4 Hz, 3H), 1.29 (s, 3H), 1.26 (s, 3H), 1.07 (s, 3H). 1H NMR (400 MHz, DMSO-d6) δ 9.19 (s, 1H), 8.40 (d, J = 8.0 Hz, 1H), 8.03 (s, 1H), 7.82 (t, J = 8.0 Hz, 1H), 7.66-7.58 (m, 3H), 6.83 (d, J = 8.8 Hz, 2H), 4.93- 4.65 (m, 3H), 4.54 (t, J = 8.4 Hz, 1H), 4.14-4.11 (m, 1H), 3.86-3.83 (m, 1H), 3.70-3.67 (m, 1H), 2.80-2.75 (m, 1H), 2.65-2.56 (m, 2H), 2.37-2.34 (m, 2H), 2.26 (s, 3H), 2.17-2.12 (m, 1H), 1.83- 1.69 (m, 1H), 1.57-1.51 (m, 1H), 1.38 (d, J = 6.0 Hz, 3H), 1.25 (s, 3H), 1.24 (s, 3H), 1.06 (s, 3H). | LCMS (ES, m/z): 586 [M + H]+, Rt 0.676 min. LCMS (ES, m/z): 586 [M + H]+, Rt 0.675 min. | Column: Chiral ART Cellulose- SA, 2*25 cm, 5 μm; Mobile Phase A: Hex(0.5% 2M NH3-MeOH)- HPLC, Mobile Phase B: EtOH- HPLC; Flow rate: 20 mL/min; Gradient: 40% B to 40% B in 15 min; Wave Length: 254/220 nm; RT1(min): 9.23; RT2(min): 11.34; |
| 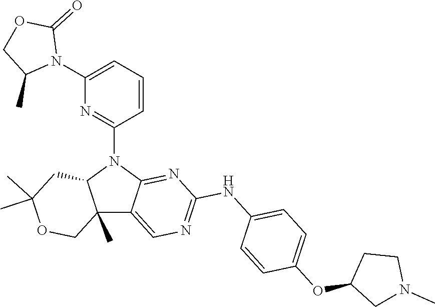 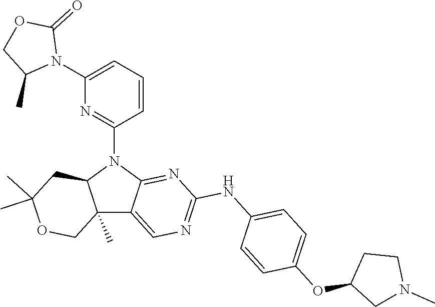 | 332, 333 | 1H NMR (400 MHz, DMSO-d6) δ 9.19 (s, 1H), 8.40 (d, J = 8.4 Hz, 1H), 8.01 (s, 1H), 7.82 (t, J = 8.4 Hz, 1H), 7.67-7.58 (m, 3H), 6.83 (d, J = 8.8 Hz, 2H), 4.82- 4.78 (m, 2H), 4.57 (, J = 8.0 Hz, 1H), 4.50-4.47 (m, 1H), 4.12-4.09 (m, 1H), 3.82-3.79 (m, 1H), 3.69-3.66 (m, 1H), 2.81-2.77 (m, 1H), 2.67-2.58 (m, 2H), 2.39-2.32 (m, 2H), 2.29-2.24 (m, 4H), 1.81-1.72 (m, 1H), 1.61-1.58 (m, 1H), 1.48 (d, J = 6.4 Hz, 3H), 1.29 (s, 3H), 1.26 (s, 3H), 1.07 (s, 3H). 1H NMR (400 MHz, DMSO-d6) δ 9.19 (s, 1H), 8.40 (d, J = 8.4 Hz, 1H), 8.03 (s, 1H), 7.82 (t, J = 8.4 Hz, 1H), 7.66-7.58 (m, 3H), 6.83 (d, J = 9.2 Hz, 2H), 4.97- 4.89 (m, 1H), 4.84-4.79 (m, 1H), 4.68- 4.65 (m, 1H), 4.54 (t, J = 8.4 Hz, 1H), 4.14-4.11 (m, 1H), 3.86-3.83 (m, 1H), 3.70-3.67 (m, 1H), 2.80-2.76 (m, 1H), 2.67-2.62 (m, 1H), 2.60-2.57 (m, 1H), 2.40-2.34 (m, 1H), 2.31-2.23 (m, 4H), 2.17-2.12 (m, 1H), 1.80-1.73 (m, 1H), 1.57-1.51 (m, 1H), 1.38 (d, J = 6.0 Hz, 3H), 1.26 (s, 3H), 1.24 (s, 3H), 1.06 (s, 3H). | LCMS (ES, m/z): 586 [M + H]+, Rt 1.350 min. LCMS (ES, m/z): 586 [M + H]+, Rt 1.325 min. | Column: Chiral ART Cellulose- SA, 2 × 25 cm, 5 μm; Mobile Phase A: Hex(0.5% 2M NH3-MeOH)- HPLC, Mobile Phase B: EtOH- HPLC(50% to 50% in 15 min); Flow rate: 20 mL/min; Wave Length: 254/220 nm; RT1(min): 8.16; RT2(min): 10.93 |
| 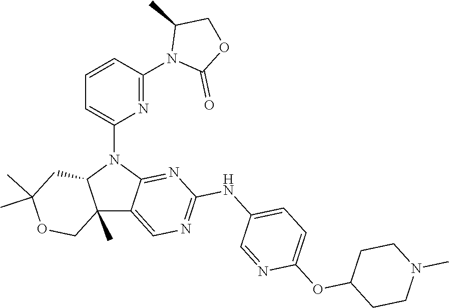 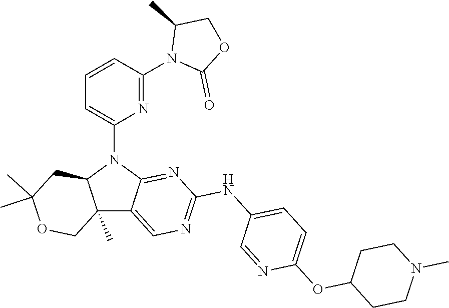 | 334, 335 | 1H NMR (300 MHz, DMSO-d6) δ 9.30 (s, 1H), 8.45-8.35 (m, 2H), 8.04-7.99 (m, 2H), 7.80 (t, J = 8.1 Hz, 1H), 7.67 (d, J = 8.1 Hz, 1H), 6.78 (d, J = 8.7 Hz, 1H), 5.03-4.89 (m, 1H), 4.85-4.73 (m, 1H), 4.62-4.45 (m, 2H), 4.14-4.05 (m, 1H), 3.86-3.76 (m, 1H), 3.73-3.63 (m, 1H), 2.78-2.72 (m, 2H), 2.40-2.26 (m, 6H), 2.05-1.95 (m, 2H), 1.80-1.53 (m, 3H), 1.48 (d, J = 6.0 Hz, 3H), 1.29 (s, 3H), 1.25 (s, 3H), 1.07 (s, 3H). 1H NMR (300 MHz, DMSO-d6) δ 9.30 (s, 1H), 8.42-8.33 (m, 2H), 8.09-7.99 (m, 2H), 7.81 (t, J = 8.1 Hz, 1H), 7.65 (d, J = 8.1 Hz, 1H), 6.77 (d, J = 8.7 Hz, 1H), 5.01-4.86 (m, 2H), 4.71-4.62 (m, 1H), 4.57-4.48 (m, 1H), 4.15-4.09 (m, 1H), 3.90-3.80 (m, 1H), 3.74-3.63 (m, 1H), 2.77-2.63 (m, 2H), 2.30-2.07 (m, 6H), 2.05-1.91 (m, 2H), 1.76-1.60 (m, 2H), 1.59-1.47 (m, 1H), 1.38 (d, J = 6.3 Hz, 3H), 1.25 (s, 3H), 1.24 (s, 3H), 1.06 (s, 3H). | LCMS (ES, m/z): 601 [M + H] +; RT: 0.687 min. LCMS (ES, m/z): 601 [M + H] +; RT: 0.694 min. | Column: CHIRALPAK IE, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 15% B to 15% B in 25 min; Wave Length: 254/220 nm; RT1(min): 18.97; RT2(min): 22.37; |
| 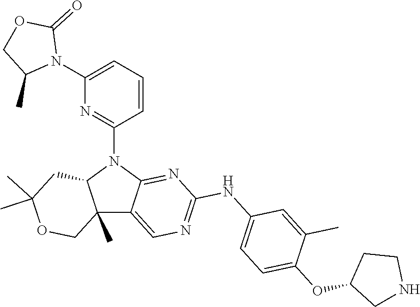 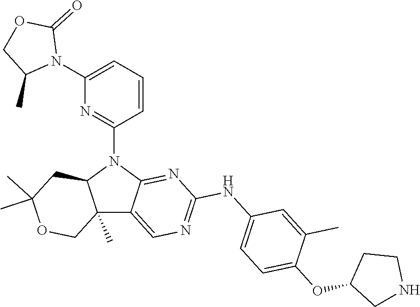 | 336, 337 | 1H NMR (300 MHz, DMSO-d6) δ 9.14 (s, 1H), 8.39 (d, J = 8.1 Hz, 1H), 8.02 (s, 1H), 7.79 (t, J = 8.1 Hz, 1H), 7.66 (d, J = 8.1 Hz, 1H), 7.52 (d, J = 2.7 Hz, 1H), 7.37 (d, J = 7.5 Hz, 1H), 6.85 (d, J = 8.7 Hz, 1H), 4.86-4.71 (m, 2H), 4.63-4.43 (m, 2H), 4.13-4.08 (m, 1H), 3.87-3.60 (m, 2H), 3.12-2.73 (m, 4H), 2.40-2.31 (m, 1H), 2.14 (s, 3H), 2.07-1.89 (m, 1H), 1.86-1.73 (m, 1H), 1.61-1.52 (m, 1H), 1.48 (d, J = 6.3 Hz, 3H), 1.28 (s, 3H), 1.25 (s, 3H), 1.07 (s, 3H). 1H NMR (300 MHz, DMSO-d6) δ 9.16 (s, 1H), 8.39 (d, J = 8.1 Hz, 1H), 8.03 (s, 1H), 7.80 (t, J = 8.1 Hz, 1H), 7.64 (d, J = 8.1 Hz, 1H), 7.52 (d, J = 2.4 Hz, 1H), 7.37 (d, J = 8.4 Hz, 1H), 6.95-6.80 (m, 1H), 4.99-4.85 (m, 1H), 4.85-4.75 (m, 1H), 4.69-4.60 (m, 1H), 4.54 (t, J = 8.1 Hz, 1H), 4.19-4.10 (m, 1H), 3.90-3.80 (m, 1H), 3.73-3.63 (m, 1H), 3.15-2.69 (m, 4H), 2.19-2.06 (m, 4H), 2.06-1.91 (m, 1H), 1.85-1.73 (m, 1H), 1.60-1.44 (m, 1H), 1.38 (d, J = 6.3 Hz, 3H), 1.25 (s, 3H), 1.24 (s, 3H), 1.06 (s, 3H). | LCMS (ES, m/z): 586 [M + H] +; RT: 0.720 min. LCMS (ES, m/z): 586 [M + H] +; RT: 0.724 min. | Column: CHIRALPAK IG, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 15% B to 15% B in 21 min; Wave Length: 220/254 nm; RT1(min): 15.11; RT2(min): 18.37; |
| 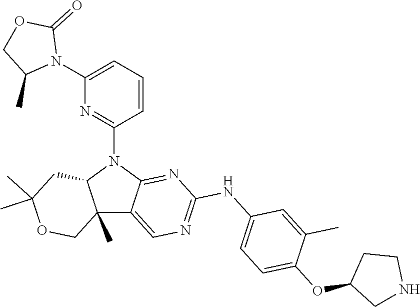 [SMILES: Cc1cc(Nc2ncc3c(n2)N(c2cccc(N4C(=O)OC[C@@H]4C)n2)[C@H]2CC(C)(C)OC[C@@]32C)ccc1O[C@H]1CCNC1] 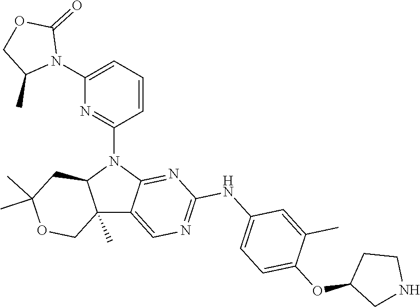 [SMILES: Cc1cc(Nc2ncc3c(n2)N(c2cccc(N4C(=O)OC[C@@H]4C)n2)[C@@H]2CC(C)(C)OC[C@]32C)ccc1O[C@H]1CCNC1] | 338, 339 | 1H NMR (300 MHz, DMSO-d6) δ 9.12 (s, 1H), 8.39 (d, J = 8.1 Hz, 1H), 8.01 (s, 1H), 7.79 (t, J = 8.1 Hz, 1H), 7.67-7.64 (m, 1H), 7.53-7.51 (m, 1H), 7.38-7.35 (m, 1H), 6.86-6.83 (m, 1H), 4.99-4.72 (m, 2H), 4.59-4.50 (m, 1H), 4.49-4.45 (m, 1H), 4.12-4.08 (m, 1H), 3.82-3.79 (m, 1H), 3.69-3.65 (m, 1H), 3.47-3.34 (m, 1H), 3.03-2.82 (m, 3H), 2.38-2.32 (m, 1H), 2.17-1.72 (m, 5H), 1.62-1.54 (m, 1H), 1.48 (d, J = 6.3 Hz, 3H), 1.28 (s, 3H), 1.25 (s, 3H), 1.07 (s, 3H). 1H NMR (400 MHz, DMSO-d6) δ 9.12 (s, 1H), 8.38 (d, J = 8.0 Hz, 1H), 8.02 (s, 1H), 7.79 (t, J = 7.2 Hz, 1H), 7.63 (d, J = 7.6 Hz, 1H), 7.51 (s, 1H), 7.37 (d, J = 6.8 Hz, 1H), 6.84 (d, J = 8.0 Hz, 1H), 4.99-4.91 (m, 1H), 4.79-4.71 (m, 1H), 4.67-4.56 (m, 1H), 4.54-4.52 (m, 1H), 4.13-4.11 (m, 1H), 3.86-3.83 (m, 1H), 3.69-3.66 (m, 1H), 3.16-3.12 (m, 1H), 3.06-3.03 (m, 1H), 2.94-2.91 (m, 1H), 2.87-2.79 (m, 1H), 2.33-2.25 (m, 4H), 2.14-1.93 (m, 1H), 1.79-1.71 (m, 1H), 1.56-1.50 (m, 1H), 1.38 (d, J = 6.0 Hz, 3H), 1.28 (s, 3H), 1.25 (s, 3H), 1.06 (s, 3H). | LCMS (ES, m/z): 586 [M + H]+, Rt 0.710 min LCMS (ES, m/z): 586 [M + H]+, Rt 0.708 min. | Column: CHIRALPAK IG, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 20% B to 20% B in 23 min; Wave Length: 220/254 nm; RT1(min): 15.25; RT2(min): 20.73; |
| 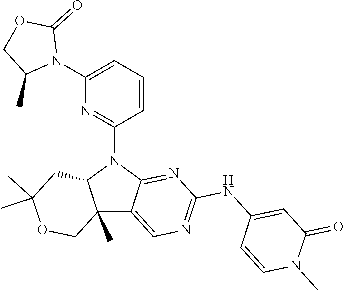 [SMILES: C[C@H]1COC(=O)N1c1cccc(N2c3nc(Nc4ccn(C)c(=O)c4)ncc3[C@]3(C)COC(C)(C)C[C@H]23)n1] 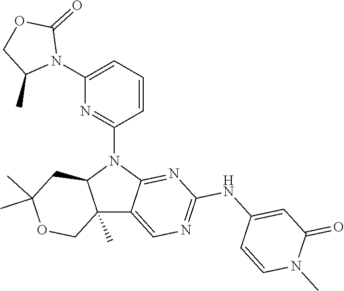 [SMILES: C[C@H]1COC(=O)N1c1cccc(N2c3nc(Nc4ccn(C)c(=O)c4)ncc3[C@@]3(C)COC(C)(C)C[C@@H]23)n1] | 340, 341 | 1H NMR (300 MHz, DMSO-d6) δ 9.62 (s, 1H), 8.46-8.34 (m, 1H), 8.16 (s, 1H), 7.85 (t, J = 8.1 Hz, 1H), 7.70 (d, J = 8.1 Hz, 1H), 7.52 (d, J = 7.5 Hz, 1H), 7.13- 7.06 (m, 1H), 6.55-6.45 (m, 1H), 4.82- 4.76 (m, 1H), 4.63-4.47 (m, 2H), 4.16- 4.06 (m, 1H), 3.89-3.79 (m, 1H), 3.75- 3.65 (m, 1H), 2.42-2.29 (m, 1H), 1.70- 1.56 (m, 1H), 1.48 (d, J = 6.3 Hz, 3H), 1.31 (s, 3H), 1.26 (s, 3H), 1.12 (d, J = 6.6 Hz, 3H), 1.07 (s, 3H). 1H NMR (300 MHz, DMSO-d6) δ 9.62 (s, 1H), 8.38 (d, J = 8.1 Hz, 1H), 8.17 (s, 1H), 7.85 (t, J = 8.1 Hz, 1H), 7.69 (d, J = 8.1 Hz, 1H), 7.52 (d, J = 7.5 Hz, 1H), 7.13-7.06 (m, 1H), 6.55-6.45 (m, 1H), 5.15-4.88 (m, 1H), 4.83-4.63 (m, 1H), 4.55 (t, J = 8.1 Hz, 1H), 4.18-4.08 (m, 1H), 3.93-3.83 (m, 1H), 3.83-3.61 (m, 1H), 2.22-2.09 (m, 1H), 1.69-1.51 (m, 1H), 1.39 (d, J = 6.3 Hz, 3H), 1.28 (s, 3H), 1.24 (s, 3H), 1.13 (d, J = 6.3 Hz, 3H), 1.06 (s, 3H). | LCMS (ES, m/z): 518 [M + H]+. Rt 0.750 min. LCMS (ES, m/z): 518 [M + H]+. Rt 0.742 min. | Column: CHIRALPAK IA, 2*25 cm, 5 μm; Mobile Phase A: MtBE(0.5% IPAmine)- HPLC- [symbol] , Mobile Phase B: EtOH-HPLC; Flow rate: 20 mL/min; Gradient: 20% B to 20% B in 19.5 min; Wave Length: 220/254 nm; RT1(min): 11.79; RT2(min): 17.33; Sample Solvent: EtOH (0.1% FA)- HPLC; Injection Volume: 1 mL; Number Of Runs: 2 |
| 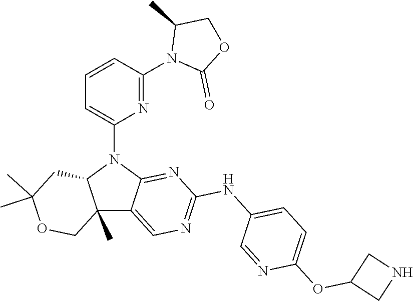 [SMILES: C[C@H]1COC(=O)N1c1cccc(N2c3nc(Nc4ccc(OC5CNC5)nc4)ncc3[C@]3(C)COC(C)(C)C[C@H]23)n1] 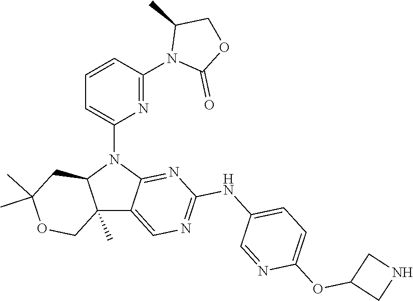 [SMILES: C[C@H]1COC(=O)N1c1cccc(N2c3nc(Nc4ccc(OC5CNC5)nc4)ncc3[C@@]3(C)COC(C)(C)C[C@@H]23)n1] | 342, 343 | 1H NMR (400 MHz, DMSO-d6) δ 9.30 (s, 1H), 8.38-8.33 (m, 2H), 8.05-8.02 (m, 2H), 7.80 (t, J = 8.4 Hz, 1H), 7.66 (d, J = 8.0 Hz, 1H), 6.81 (d, J = 8.8 Hz, 1H), 5.30-5.27 (m, 1H), 4.87-4.71 (m, 1H), 4.59-4.47 (m, 2H), 4.11-4.08 (m, 1H), 3.82-3.66 (m, 4H), 3.52-3.49 (m, 2H), 2.36-2.32 (m, 1H), 1.61-1.55 (m, 1H), 1.47 (d, J = 6.0 Hz, 3H), 1.28 (s, 3H), 1.25 (s, 3H), 1.06 (s, 3H). 1H NMR (300 MHz, DMSO-d6) δ 9.30 (s, 1H), 8.38-8.32 (m, 2H), 8.06-8.03 (m, 2H), 7.80 (t, J = 8.4 Hz, 1H), 7.64 (d, J = 7.8 Hz, 1H), 6.80 (d, J = 8.7 Hz, 1H), 5.30-5.26 (m, 1H), 4.96-4.92 (m, 1H), 4.69-4.64 (m, 1H), 4.53 (t, J = 8.4 Hz, 1H), 4.13-4.09 (m, 1H), 3.89-3.81 (m, 1H), 3.77-3.70 (m, 3H), 3.66-3.49 (m, 2H), 2.15-2.10 (m, 1H), 1.57-1.45 (m, 1H), 1.37 (d, J = 6.3 Hz, 3H), 1.28 (s, 3H), 1.25 (s, 3H), 1.06 (s, 3H). | LCMS (ES, m/z): 559 [M + H]+, Rt 0.675 min LCMS (ES, m/z): 559 [M + H]+, Rt 1.273 min. | Column: CHIRALPAK IE, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 15% B to 15% B in 28 min; Wave Length: 254/220 nm; RT1(min): 20.61; RT2(min): 24.91; |
| 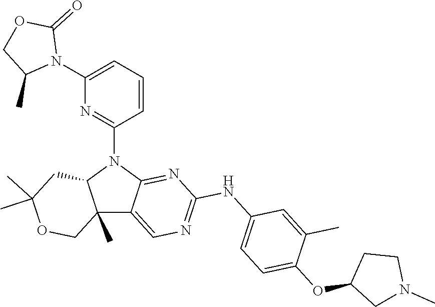 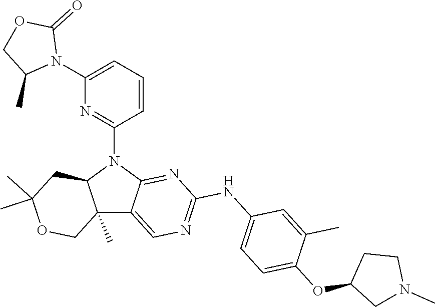 | 344, 345 | 1H NMR (300 MHz, DMSO-d6) δ 9.13 (s, 1H), 8.39 (d, J = 8.4 Hz, 1H), 8.01 (s, 1H), 7.79 (t, J = 8.1 Hz, 1H), 7.65 (d, J = 7.8 Hz, 1H), 7.51 (d, J = 2.7 Hz, 1H), 7.38-7.35 (m, 1H), 6.79 (d, J = 8.7 Hz, 1H), 4.81-4.78 (m, 2H), 4.57 (t, J = 8.4 Hz, 1H), 4.49-4.47 (m, 1H), 4.12-4.08 (m, 1H), 3.82-3.78 (m, 1H), 3.69-3.65 (m, 1H), 2.81-2.78 (m, 1H), 2.66-2.51 (m, 2H), 2.40-2.25 (m, 6H), 2.14 (s, 3H), 1.86-1.73 (m, 1H), 1.60-1.56 (m, 1H), 1.48 (d, J = 6.0 Hz, 3H), 1.28 (s, 3H), 1.25 (s, 3H), 1.06 (s, 3H). 1H NMR (300 MHz, DMSO-d6) δ 9.13 (s, 1H), 8.39 (d, J = 8.1 Hz, 1H), 8.03 (s, 1H), 7.80 (t, J = 8.1 Hz, 1H), 7.64 (d, J = 8.1 Hz, 1H), 7.51 (d, J = 2.1 Hz, 1H), 7.38-7.34 (m, 1H), 6.78 (d, J = 9.0 Hz, 1H), 4.91-4.80 (m, 1H), 4.81-4.78 (m, 1H), 4.69-4.65 (m, 1H), 4.54 (t, J = 8.4 Hz, 1H), 4.14-4.10 (m, 1H), 3.86-3.82 (m, 1H), 3.69-3.65 (m, 1H), 2.83-2.78 (m, 1H), 2.66-2.51 (m, 2H), 2.40-2.38 (m, 1H), 2.29-2.25 (m, 4H), 2.17-2.11 (m, 4H), 1.86-1.73 (m, 1H), 1.60-1.56 (m, 1H), 1.38 (d, J = 6.3 Hz, 3H), 1.25 (s, 3H), 1.24 (s, 3H), 1.06 (s, 3H). | LCMS (ES, m/z): 600 [M + H]+, Rt 0.715 min. LCMS (ES, m/z): 600 [M + H]+, Rt 0.704 min. | Column: CHIRALPAK IG, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 20% B to 20% B in 23 min; Wave Length: 220/254 nm; RT1(min): 15.25; RT2(min): 20.73 |
| 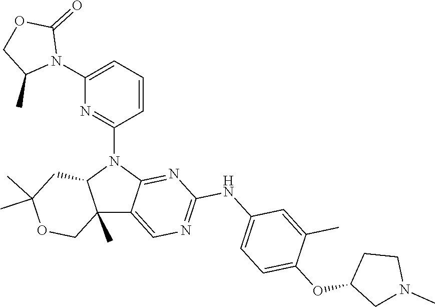 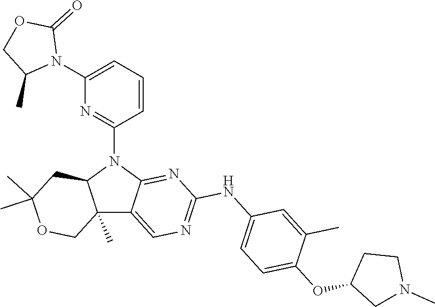 | 346, 347 | 1H NMR (300 MHz, DMSO-d6) δ 9.14 (s, 1H), 8.39 (d, J = 8.1 Hz, 1H), 8.02 (s, 1H), 7.79 (t, J = 8.1 Hz, 1H), 7.66 (d, J = 8.1 Hz, 1H), 7.52 (d, J = 2.7 Hz, 1H), 7.39-7.33 (m, 1H), 6.79 (d, J = 8.7 Hz, 1H), 4.87-4.72 (m, 2H), 4.61-4.44 (m, 2H), 4.15-4.08 (m, 1H), 3.86-3.76 (m, 1H), 3.72-3.62 (m, 1H), 2.88-2.80 (m, 1H), 2.75-2.56 (m, 2H), 2.48-2.18 (m, 2H), 2.35-2.20 (m, 4H), 2.15 (s, 3H), 1.88-1.72 (m, 1H), 1.61-1.53 (m, 1H), 1.48 (d, J = 6.3 Hz, 3H), 1.28 (s, 3H), 1.25 (s, 3H), 1.07 (s, 3H). 1H NMR (300 MHz, DMSO-d6) δ 9.13 (s, 1H), 8.38 (d, J = 8.1 Hz, 1H), 8.03 (s, 1H), 7.80 (t, J = 8.1 Hz, 1H), 7.64 (d, J = 8.1 Hz, 1H), 7.52 (d, J = 2.7 Hz, 1H), 7.39-7.33 (m, 1H), 6.79 (d, J = 8.7 Hz, 1H), 4.99-4.86 (m, 1H), 4.86-4.74 (m, 1H), 4.69-4.62 (m, 1H), 4.54 (t, J = 8.1 Hz, 1H), 4.15-4.08 (m, 1H), 3.90-3.80 (m, 1H), 3.73-3.63 (m, 1H), 2.88-2.80 (m, 1H), 2.73-2.54 (m, 2H), 2.45-2.35 (m, 1H), 2.33-2.22 (m, 4H), 2.20-2.10 (m, 4H), 1.87-1.71 (m, 1H), 1.57-1.49 (m, 1H), 1.38 (d, J = 6.3 Hz, 3H), 1.25 (s, 3H), 1.24 (s, 3H), 1.06 (s, 3H). | LCMS (ES, m/z): 600 [M + H] +; RT: 0.726 min. LCMS (ES, m/z): 600 [M + H] +; RT: 0.727 min. | Column: CHIRALPAK IG, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 15% B to 15% B in 21 min; Wave Length: 220/254 nm; RT1(min): 15.11; RT2(min): 18.37; |
| 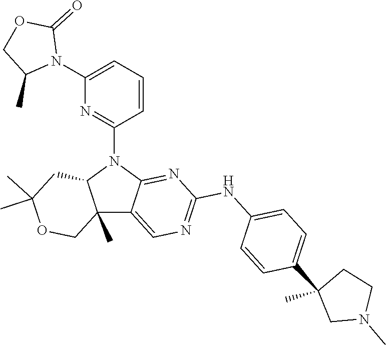 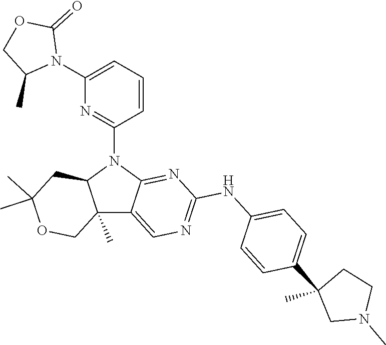 | 348, 349 | 1H NMR (300 MHz, DMSO-d6) δ 9.31 (s, 1H), 8.43 (d, J = 8.1 Hz, 1H), 8.04 (s, 1H), 7.82 (t, J = 8.1 Hz, 1H), 7.71-7.58 (m, 3H), 7.23 (d, J = 8.7 Hz, 2H), 4.85- 4.71 (m, 1H), 4.65-4.38 (m, 2H), 4.15- 4.05 (m, 1H), 3.86-3.62 (m, 2H), 2.84- 2.58 (m, 3H), 2.47-2.37 (m, 1H), 2.36- 2.26 (m, 4H), 2.20-1.85 (m, 2H), 1.69- 1.51 (m, 1H), 1.48 (d, J = 6.2 Hz, 3H), 1.36 (s, 3H), 1.29 (s, 3H), 1.25 (s, 3H), 1.07 (s, 3H). 1H NMR (300 MHz, DMSO-d6) δ 9.31 (s, 1H), 8.43 (d, J = 8.1 Hz, 1H), 8.05 (s, 1H), 7.83 (t, J = 8.1 Hz, 1H), 7.69-7.59 (m, 3H), 7.22 (d, J = 8.7 Hz, 2H), 4.96- 4.90 (m, 1H), 4.73-4.62 (m, 1H), 4.54 (t, J = 8.1 Hz, 1H), 4.17-4.07 (m, 1H), 3.91-3.81 (m, 1H), 3.73-3.63 (m, 1H), 2.85-2.57 (m, 3H), 2.51- 2.40 (m, 1H), 2.29 (s, 3H), 2.20-1.81 (m, 3H), 1.61- 1.47 (m, 1H), 1.38 (d, J = 6.3 Hz, 3H), 1.36 (s, 3H), 1.26 (s, 3H), 1.24 (s, 3H), 1.06 (s, 3H). | LCMS (ES, m/z): 584 [M + H]+. Rt 0.683 min. LCMS (ES, m/z): 584 [M + H]+. Rt 0.683 min. | Column: CHIRALPAK IG, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 50% B to 50% B in 12.5 min; Wave Length: 220/254 nm; RT1(min): 9.06; RT2(min): 11.40; Sample Solvent: EtOH- HPLC; Injection Volume: 0.8 mL; Number Of Runs: 10 |
| 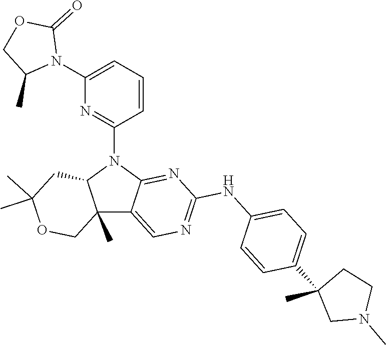 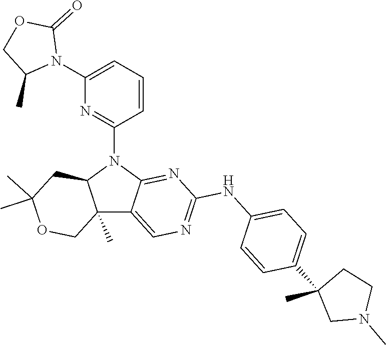 | 350, 351 | 1H NMR (300 MHz, DMSO-d6) δ 9.28 (s, 1H), 8.43 (d, J = 8.1 Hz, 1H), 8.04 (s, 1H), 7.82 (t, J = 8.1 Hz, 1H), 7.65 (t, J = 8.7 Hz, 3H), 7.23 (d, J = 8.7 Hz, 2H), 4.85-4.69 (m, 1H), 4.59-4.46 (m, 2H), 4.12-4.08 (m, 1H), 3.83-3.79 (m, 1H), 3.70-3.66 (m, 1H), 2.85-2.70 (m, 1H), 2.70-2.61 (m, 2H), 2.45-2.30 (m, 5H), 2.19-2.03 (m, 1H), 1.95-1.81 (m, 1H), 1.63-1.55 (m, 1H), 1.48 (d, J = 6.3 Hz, 3H), 1.36 (s, 3H), 1.29 (s, 3H), 1.25 (s, 3H), 1.07 (s, 3H). 1H NMR (300 MHz, DMSO-d6) δ 9.31 (s, 1H), 8.44 (d, J = 8.4 Hz, 1H), 8.07 (s, 1H), 7.84 (t, J = 8.1 Hz, 1H), 7.67-7.63 (m, 3H), 7.24 (d, J = 8.7 Hz, 2H), 4.99- 4.87 (m, 1H), 4.71-4.66 (m, 1H), 4.55 (t, J = 8.4 Hz, 1H), 4.15-4.11 (m, 1H), 3.89-3.85 (m, 1H), 3.72-3.68 (m, 1H), 2.82-2.62 (m, 4H), 2.31 (s, 3H), 2.19- 2.10 (m, 2H), 1.95-1.85 (m, 1H), 1.59- 1.52 (m, 1H), 1.40 (s, 3H), 1.37 (s, 3H), 1.27 (s, 3H), 1.25 (s, 3H),1.07 (s, 3H). | LCMS (ES, m/z): 584 [M + H]+, Rt 0.671 min. LCMS (ES, m/z): 584 [M + H]+, Rt 0.664 min. | Column: CHIRALPAK IG, 2*25 cm, 5 μm; Mobile Phase A: Hex: DCM = 3:1 (0.5% 2M NH3-MeOH)- HPLC, Mobile Phase B: EtOH- HPLC; Flow rate: 20 mL/min; Gradient: 30% B to 30% B in 13 min; Wave Length: 220/254 nm; RT1(min): 9.62; RT2(min): 11.90; Sample Solvent: EtOH- HPLC; Injection Volume: 1 mL; Number Of Runs: 10 |
| 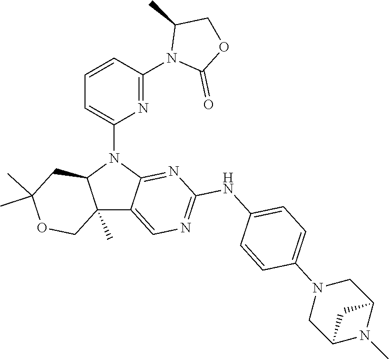 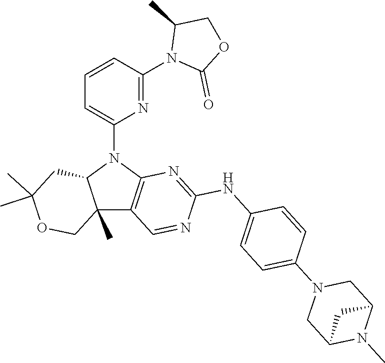 | 352, 353 | 1H NMR (300 MHz, DMSO-d6) δ 9.01 (s, 1H), 8.43 (d, J = 8.1 Hz, 1H), 7.99 (s, 1H), 7.81 (t, J = 8.1 Hz, 1H), 7.66-7.49 (m, 3H), 6.70 (d, J = 9.0 Hz, 2H), 4.99- 4.87 (m, 1H), 4.71-4.60 (m, 1H), 4.54 (t, J = 8.1 Hz, 1H), 4.10-4.15 (m, 1H), 3.89-3.79 (m, 1H), 3.72-3.62 (m, 1H), 3.57 (d, J = 5.7 Hz, 2H), 3.46-3.35 (m, 2H), 3.33-3.21 (m, 2H), 2.48-2.36 (m, 1H), 2.20-2.08 (m, 1H), 1.99 (s, 3H), 1.60-1.44 (m, 2H), 1.38 (d, J = 6.3 Hz, 3H), 1.25 (s, 3H), 1.24 (s, 3H), 1.06 (s, 3H). 1H NMR (300 MHz, DMSO-d6) δ 9.01 (s, 1H), 8.43 (d, J = 8.1 Hz, 1H), 7.98 (s, 1H), 7.80 (t, J = 8.1 Hz, 1H), 7.64 (d, J = 8.1 Hz, 1H), 7.52 (d, J = 8.7 Hz, 2H), 6.69 (d, J = 8.7 Hz, 2H), 4.81-4.75 (m, 1H), 4.56 (t, J = 8.1 Hz, 1H), 4.51-4.41 (m, 1H), 4.15-4.10 (m, 1H), 3.85-3.75 (m, 1H), 3.72-3.62 (m, 1H), 3.61-3.48 (m, 2H), 3.47-3.38 (m, 2H), 3.31-3.21 (m, 2H), 2.46-2.28 (m, 2H), 1.99 (s, 3H), 1.63-1.44 (m, 5H), 1.28 (s, 3H), 1.25 (s, 3H), 1.07 (s, 3H). | LCMS (ES, m/z): 597 [M + H] +; RT: 0.653 min. LCMS (ES, m/z): 597 [M + H] +; RT: 0.660 min. | Column: CHIRAL ART Amylose-SA, 2*25 cm, 5 μm; Mobile Phase A: MtBE(0.5% IPAmine)- HPLC, Mobile Phase B: IPA- HPLC; Flow rate: 20 mL/min; Gradient: 30% B to 30% B in 9 min; Wave Length: 254/220 nm; RT1(min): 6.97; RT2(min): 8.97; |
| 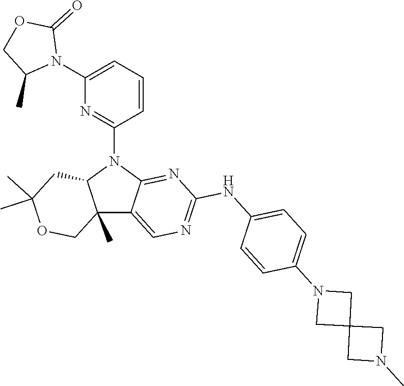 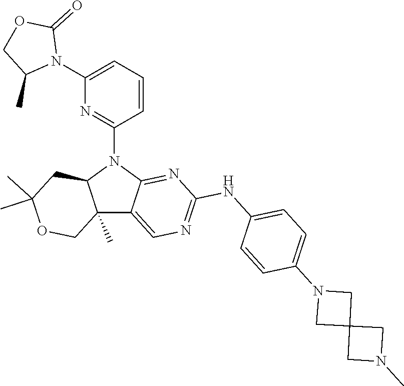 | 354, 355 | 1H NMR (400 MHz, DMSO-d6) δ 9.02 (s, 1H), 8.40 (d, J = 7.6 Hz, 1H), 7.97 (s, 1H), 7.76 (t, J = 8.0 Hz, 1H), 7.65 (d, J = 7.6 Hz, 1H), 7.46 (d, J = 8.8 Hz, 2H), 6.40 (d, J = 8.8 Hz, 2H), 4.78-4.76 (m, 1H), 4.56 (t, J = 8.0 Hz, 1H), 4.47-4.44 (m, 1H), 4.11-4.08 (m, 1H), 3.80-3.77 (m, 5H), 3.67-3.64 (m, 1H), 3.32-3.24 (m, 4H), 2.50-2.49 (m, 1H), 2.18 (s, 3H), 1.59-1.53 (m, 1H), 1.47 (d, J = 6.0 Hz, 3H), 1.27 (s, 3H), 1.24 (s, 3H), 1.06 (s, 3H). 1H NMR (400 MHz, DMSO-d6) δ 9.03 (s, 1H), 8.40 (d, J = 8.0 Hz, 1H), 7.98 (s, 1H), 7.79 (t, J = 8.0 Hz, 1H), 7.63 (d, J = 7.6 Hz, 1H), 7.46 (d, J = 8.4 Hz, 2H), 6.40 (d, J = 8.8 Hz, 2H), 4.94-4.90 (m, 1H), 4.66-4.62 (m, 1H), 4.55-4.51 (m, 1H), 4.12-4.10 (m, 1H), 3.84-3.79 (m, 5H), 3.68-3.65 (m, 1H), 3.32-3.24 (m, 4H), 2.18-2.11 (m, 4H), 1.54-1.49 (m, 1H), 1.37 (d, J = 6.4 Hz, 3H), 1.23 (s, 6H), 1.05 (s, 3H). | LCMS (ES, m/z): 597 [M + H]+. Rt 0.651 min. LCMS (ES, m/z): 597 [M + H]+. Rt 0.655 min | Column: XBridge Prep OBD C18 Column, 30*150 mm, 5 μm; Mobile Phase A: Water(10 mmol/L NH4HCO3 + 0.1% NH3.H2O), Mobile Phase B: ACN; Flow rate: 60 mL/min; Gradient: 40% B to 60% B in 7 min, 60% B; Wave Length: 254 nm; RT1(min): 5.53; Number Of Runs: 0 |
| 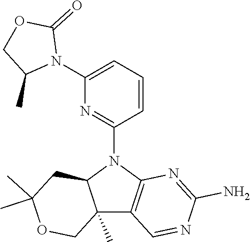 [SMILES: C[C@H]1COC(=O)N1c1cccc(N2c3nc(N)ncc3[C@@]3(C)COC(C)(C)C[C@@H]23)n1] 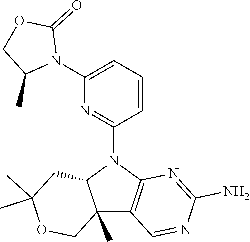 [SMILES: C[C@H]1COC(=O)N1c1cccc(N2c3nc(N)ncc3[C@]3(C)COC(C)(C)C[C@H]23)n1] | 356, 357 | 1H NMR (300 MHz, DMSO-d6) δ 8.41 (d, J = 8.1 Hz, 1H), 7.85 (s, 1H), 7.77 (t, J = 8.1 Hz, 1H), 7.60 (d, J = 7.8 Hz, 1H), 6.48-6.45 (s, 2H), 4.94-4.88 (m, 1H), 4.63-4.58 (m, 1H), 4.53 (t, J = 8.4 Hz, 1H), 4.12-4.09 (m, 1H), 3.81-3.77 (m, 1H), 3.66-3.62 (m, 1H), 2.15-2.07 (m, 1H), 1.51-1.47 (m, 1H), 1.38 (d, J = 6.0 Hz, 3H), 1.28 (s, 3H), 1.25 (s, 3H), 1.05 (s, 3H). 1H NMR (300 MHz, DMSO-d6) δ 8.41 (d, J = 8.1 Hz, 1H), 7.83 (s, 1H), 7.77 (t, J = 8.4 Hz, 1H), 7.61 (d, J = 7.8 Hz, 1H), 6.46-6.44 (m, 2H), 4.80-4.74 (m, 1H), 4.58-4.53 (m, 1H), 4.44-4.39 (m, 1H), 4.11-4.07 (m, 1H), 3.77-3.73 (m, 1H), 3.65-3.61 (m, 1H), 2.36-2.29 (m, 1H), 1.55-1.50 (m, 1H), 1.46 (d, J = 6.3 Hz, 3H), 1.28 (s, 3H), 1.25 (s, 3H), 1.05 (s, 3H). | LCMS (ES, m/z): 411 [M + H]+, Rt 0.666 min LCMS (ES, m/z): 411 [M + H]+, Rt 0.669 min. | Column: CHIRAL ART Cellulose-SA, 2*25 cm, 5 μm; Mobile Phase A: MtBE(0.3% IPA)-HPLC, Mobile Phase B: IPA-HPLC; Flow rate: 20 mL/min; Gradient: 70% B to 70% B in 7 min; Wave Length: 220/254 nm; RT1(min): 5.032; RT2(min): 5.987; Sample Solvent: IPA- HPLC; Injection Volume: 0.8 mL; Number Of Runs: 7 |
| 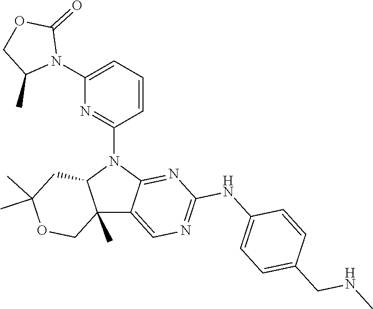 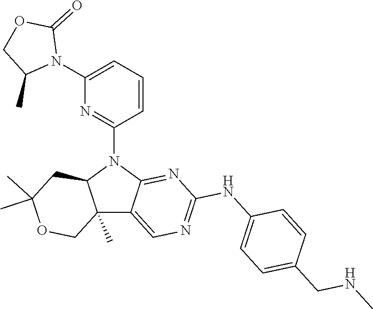 | 359, 360 | 1H NMR (300 MHz, DMSO-d6) δ 9.34 (s, 1H), 8.42 (d, J = 8.1 Hz, 1H), 8.05 (s, 1H), 7.82 (t, J = 8.1 Hz, 1H), 7.67-7.64 (m, 3H), 7.23-7.20 (m, 2H), 4.81-4.80 (m, 1H), 4.49-4.46 (m, 2H), 4.12-4.08 (m, 1H), 3.83-3.79 (m, 1H), 3.70-3.66 (m, 1H), 3.57 (s, 2H), 2.37-2.31 (m, 1H), 2.26 (s, 3H), 1.62-1.55 (m, 1H), 1.47 (d, J = 6.0 Hz, 3H), 1.25(s, 3H), 1.23 (s, 3H), 1.07 (s, 3H). 1H NMR (300 MHz, DMSO-d6) δ 9.34 (s, 1H), 8.41 (d, J = 8.4 Hz, 1H), 8.06 (s, 1H), 7.82 (t, J = 8.1 Hz, 1H), 7.67-7.63 (m, 3H), 7.23-7.20 (m, 2H), 4.98-4.86 (m, 1H), 4.68-4.65 (m, 1H), 4.53 (t, J = 8.4 Hz, 1H), 4.14-4.10 (m, 1H), 3.87- 3.83 (m, 1H), 3.70-3.66 (m, 1H), 3.57 (s, 2H), 2.26 (s, 3H), 2.17-2.11 (m, 1H), 1.54-1.51 (m, 1H), 1.38 (d, J = 6.3 Hz, 3H), 1.25 (s, 3H), 1.24 (s, 3H), 1.07 (s, 3H). | LCMS (ES, m/z): 530 [M + H]+, Rt 1.180 min. LCMS (ES, m/z): 530 [M + H]+, Rt 0.674 min. | Column: CHIRALPAK IE, 2*25 cm, 5 μm; Mobile Phase A: Hex (0.5% 2M NH3-MeOH)- HPLC, Mobile Phase B: EtOH- HPLC; Flow rate: 20 mL/min; Gradient: 40% B to 40% B in 27 min; Wave Length: 220/254 nm; RT1(min): 19.044; RT2(min): 23.594; Sample Solvent: EtOH- HPLC; Injection Volume: 0.75 mL; Number Of Runs: 6 |

[symbol]

indicates data missing or illegible when filed

### Examples 711 and 712: Dimethyl((3-((4bR,8aR)-4b,7,7-trimethyl-2-((4-((1S,4S)-5-methyl-2,5-diazabicyclo[2.2.1]heptan-2-yl)phenyl)amino)-4b,7,8,8a-tetrahydropyrano[3′,4′:4,5]pyrrolo[2,3-d]pyrimidin-9(5H)-yl)phenyl)imino)-l6-sulfanone and dimethyl((3-((4bS,8aS)-4b,7,7-trimethyl-2-((4-((1S,4S)-5-methyl-2,5-diazabicyclo[2.2.1]heptan-2-yl)phenyl)amino)-4b,7,8,8a-tetrahydropyrano[3′,4′:4,5]pyrrolo[2,3-d]pyrimidin-9(5H)-yl)phenyl)imino)-l6-sulfanone

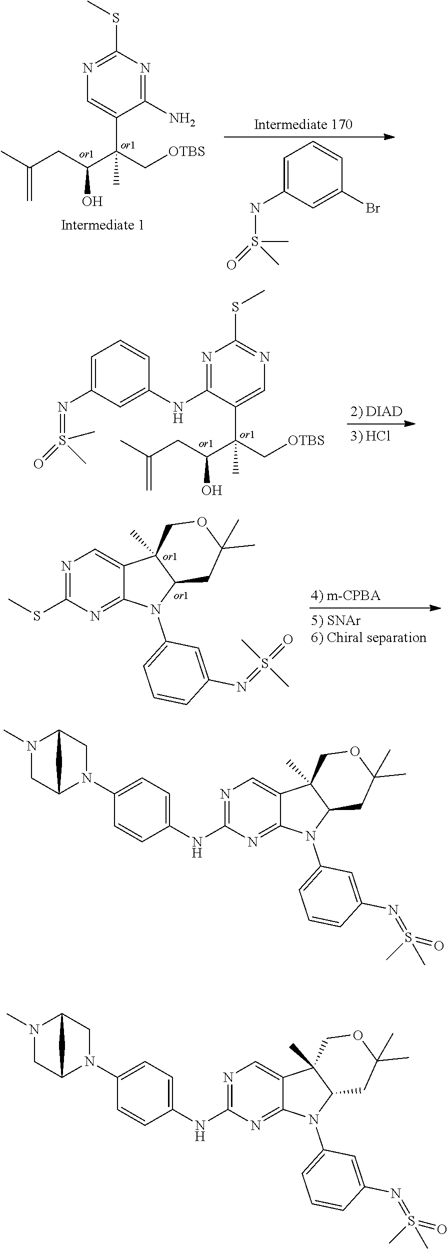

### Step 1. rel-((3-((5-((2R,3R)-1-((tert-butyldimethylsilyl)oxy)-3-hydroxy-2,5-dimethylhex-5-en-2-yl)-2-(methylthio)pyrimidin-4-yl)amino)phenyl)imino)dimethyl-l6-sulfanone

To a stirred solution of rel-(2S,3S)-2-(4-amino-2-(methylthio)pyrimidin-5-yl)-1-((tert-butyldimethylsilyl)oxy)-2,5-dimethylhex-5-en-3-ol (Intermediate 1, 0.6 g, 1.51 mmol, 1 eq.) and ((3-bromophenyl)imino)dimethyl-l6-sulfanone (Intermediate 170, 561.61 mg, 2.26 mmol, 1.5 eq.) in dioxane (5 mL) was added Brettphos Pd (136.70 mg, 150.89 μmol, 0.1 eq.), Brettphos (161.75 mg, 301.77 μmol, 0.2 eq.) and Cs₂CO₃ (983.78 mg, 3.02 mmol, 2 eq.). The reaction was stirred for 2 h at 100° C. under the N₂ atmosphere. The mixture was allowed to cool down to room temperature and the solids were filtered out and washed with EA (2×10 mL). The filtrate was concentrated under reduced pressure. The residue was purified by silica gel column chromatography (eluting with 1.5:1 EA/PE) to afford rel-((3-((5-((2R,3R)-1-((tert-butyldimethylsilyl)oxy)-3-hydroxy-2,5-dimethylhex-5-en-2-yl)-2-(methylthio)pyrimidin-4-yl)amino)phenyl)imino)dimethyl-l6-sulfanone (560 mg, 991.37 μmol, 66% yield) as yellow oil. LCMS (ES, m/z): 565 [M+H]⁺, Rt 0.720 min.

### Step 2-3. rel-dimethyl((3-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)phenyl)imino)-l6-sulfanone

Following the experimental procedures described for Example 111 and Example 112/step 2-3, rel-dimethyl((3-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)phenyl)imino)-l6-sulfanone (230 mg, 0.53 mmol) was obtained as a yellow oil, starting from rel-((3-((5-((2R,3R)-1-((tert-butyldimethylsilyl)oxy)-3-hydroxy-2,5-dimethylhex-5-en-2-yl)-2-(methylthio)pyrimidin-4-yl)amino)phenyl)imino)dimethyl-l6-sulfanone. LCMS (ES, m/z): 433 [M+H]⁺, Rt 0.568 min.

### Step 4-6. Dimethyl((3-((4bR,8aR)-4b,7,7-trimethyl-2-((4-((1S,4S)-5-methyl-2,5-diazabicyclo[2.2.1]heptan-2-yl)phenyl)amino)-4b,7,8,8a-tetrahydropyrano[3′,4′:4,5]pyrrolo[2,3-d]pyrimidin-9(5H)-yl)phenyl)imino)-l6-sulfanone and dimethyl((3-((4bS,8aS)-4b,7,7-trimethyl-2-((4-((1S,4S)-5-methyl-2,5-diazabicyclo[2.2.1]heptan-2-yl)phenyl)amino)-4b,7,8,8a-tetrahydropyrano[3′,4′:4,5]pyrrolo[2,3-d]pyrimidin-9(5H)-yl)phenyl)imino)-l6-sulfanone

Following the experimental procedures described for Example 111 and Example 112/steps 4-6, dimethyl((3-((4bR,8aR)-4b,7,7-trimethyl-2-((4-((1S,4S)-5-methyl-2,5-diazabicyclo[2.2.1]heptan-2-yl)phenyl)amino)-4b,7,8,8a-tetrahydropyrano[3′,4′:4,5]pyrrolo[2,3-d]pyrimidin-9(5H)-yl)phenyl)imino)-l6-sulfanone and dimethyl((3-((4bS,8aS)-4b,7,7-trimethyl-2-((4-((1S,4S)-5-methyl-2,5-diazabicyclo[2.2.1]heptan-2-yl)phenyl)amino)-4b,7,8,8a-tetrahydropyrano[3′,4′:4,5]pyrrolo[2,3-d]pyrimidin-9(5H)-yl)phenyl)imino)-l6-sulfanone were obtained as solids upon chiral separation. The Chiral-HPLC condition was (Column: CHIRALPAK IE, 2*25 cm/5 μm; Mobile Phase A: MtBE (0.5% IPAmine), Mobile Phase B: EtOH; Flow rate: 20 mL/min; Gradient: 10% B in 27 min; Wave Length: 220/254 nm; RT1(min): 20.98; RT2(min): 26.24).

Examples 711 and 712: ¹H NMR (400 MHz, DMSO-d6) δ 8.67 (s, 1H), 7.84 (s, 1H), 7.49-7.40 (m, 3H), 7.19 (t, J=8.1 Hz, 1H), 7.09-7.03 (m, 1H), 6.74-6.67 (m, 1H), 6.53-6.44 (m, 2H), 4.33-4.18 (m, 2H), 3.72-3.49 (m, 3H), 3.36-3.43 (m, 2H), 3.24-3.19 (m, 6H), 3.12-3.05 (m, 1H), 2.79-2.71 (m, 1H), 2.24 (s, 3H), 1.90-1.60 (m, 4H), 1.30 (s, 3H), 1.15 (s, 3H), 1.01 (s, 3H). LCMS (ES, m/z): 588 [M+H]⁺; RT: 0.577 min.

¹H NMR (400 MHz, DMSO-d6) δ 8.67 (s, 1H), 7.84 (s, 1H), 7.48-7.40 (m, 3H), 7.19 (t, J=8.1 Hz, 1H), 7.05 (d, J=2.4 Hz, 1H), 6.74-6.67 (m, 1H), 6.52-6.43 (m, 2H), 4.33-4.17 (m, 2H), 3.71-3.49 (m, 3H), 3.35-3.27 (m, 2H), 3.24-3.18 (m, 6H), 3.12-3.05 (m, 1H), 2.78-2.71 (m, 1H), 2.23 (s, 3H), 1.90-1.58 (m, 4H), 1.29 (s, 3H), 1.15 (s, 3H), 1.01 (s, 3H). LCMS (ES, m/z): 588 [M+H]⁺; RT: 0.579 min.

TABLE 16
Using appropriate intermediates and reaction conditions, the following
examples were prepared in the similar manner to Examples 711/712.

| Structure | EX # | ¹H NMR | LCMS |
|---|---|---|---|
| 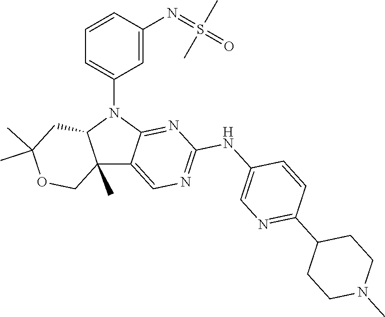 | 709, 710 | 1H NMR (300 MHz, DMSO-d6) δ 9.12 (s, 1H), 8.65 (d, J = 2.4 Hz, 1H), 8.06-8.03 (m, 1H), 7.85 (s, 1H), 7.27 (d, J = 8.7 Hz, 1H), 7.15 (t, J = 8.1 Hz, 1H), 7.08-7.00 (m, 2H), 6.68 (d, J = 7.8 Hz, 1H), 4.26 (t, J = 5.4 Hz, 1H), 3.68-3.59 (m, 1H), 3.51-3.44 (m, 2H), 3.17 (s, 3H), 3.16 (s, 3H), 2.83-2.75 (m, 2H), 2.13 (s, 3H), 1.94-1.88 (m, 2H), 1.80-1.56 (m, 6H), 1.25 (s, 3H), 1.08 (s, 3H), 0.95 (s, 3H). | LCMS (ES, m/z): 576 [M + H]+, Rt 0.619 min. |
| 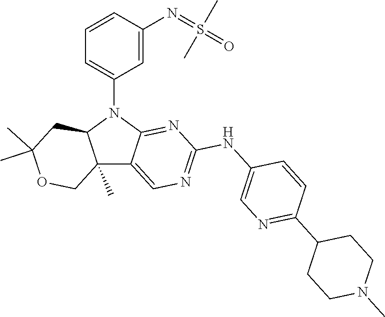 | | 1H NMR (300 MHz, DMSO-d6) δ 9.12 (s, 1H), 8.65 (d, J = 2.4 Hz, 1H), 8.07-8.03 (m, 1H), 7.85 (s, 1H), 7.27 (d, J = 9.3 Hz, 1H), 7.15 (t, J = 8.1 Hz, 1H), 7.08-7.00 (m, 2H), 6.70-6.67 (m, 1H), 4.26 (t, J = 5.4 Hz, 1H), 3.65-3.54 (m, 1H), 3.51-3.38 (m, 2H), 3.16 (d, J = 3.3 Hz, 6H), 2.88-2.75 (m, 2H), 2.13 (s, 3H), 1.95-1.91 (m, 2H), 1.80-1.56 (m, 6H), 1.25 (s, 3H), 1.08 (s, 3H), 0.95 (s, 3H). | LCMS (ES, m/z): 576 [M + H]+, Rt 0.615 min. |
| 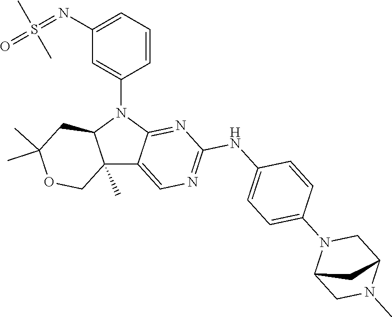 [SMILES: CN1C[C@H]2C[C@@H]1CN2c1ccc(Nc2ncc3c(n2)N(c2cccc(N=S(C)(C)=O)c2)[C@@H]2CC(C)(C)OC[C@]32C)cc1] | 713, 714 | 1H NMR (300 MHz, DMSO-d6) δ 8.64 (s, 1H), 7.83 (s, 1H), 7.46-7.42 (m, 3H), 7.18 (t, J = 8.1 Hz, 1H), 7.06 (s, 1H), 6.70 (d, J = 8.1 Hz, 1H), 6.48 (d, J = 9.0 Hz, 2H), 4.30-4.27 (m, 1H), 4.19 (s, 1H), 3.68-3.64 (m, 1H), 3.57-3.50 (m, 1H), 3.33-3.18 (m, 7H), 3.10- 3.07 (m, 1H), 2.75-2.73 (m, 1H), 2.59-2.46 (m, 1H), 2.23 (s, 3H), 1.87-1.81 (m, 2H), 1.75-1.72 (m, 1H), 1.66-1.61 (m, 1H), 1.27- 1.20 (m, 4H), 1.15 (s, 3H), 1.02 (s, 3H). | LCMS (ES, m/z): 588 [M + H]+, Rt 0.566 min. |
| 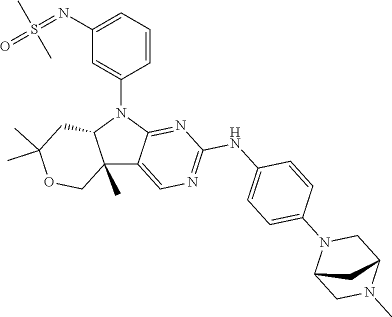 [SMILES: CN1C[C@H]2C[C@@H]1CN2c1ccc(Nc2ncc3c(n2)N(c2cccc(N=S(C)(C)=O)c2)[C@H]2CC(C)(C)OC[C@@]32C)cc1] | | 1H NMR (300 MHz, DMSO-d6) δ 8.64 (s, 1H), 7.83 (s, 1H), 7.46-7.42 (m, 3H), 7.18 (t, J = 8.1 Hz, 1H), 7.06 (s, 1H), 6.70 (d, J = 8.1 Hz, 1H), 6.48 (d, J = 9.0 Hz, 2H), 4.30-4.27 (m, 1H), 4.19 (s, 1H), 3.68-3.64 (m, 1H), 3.57-3.50 (m, 1H), 3.33-3.18 (m, 7H), 3.10- 3.07 (m, 1H), 2.75-2.73 (m, 1H), 2.59-2.46 (m, 1H), 2.23 (s, 3H), 1.87-1.81 (m, 2H), 1.5- 1.72 (m, 1H), 1.66-1.61 (m, 1H), 1.27-1.20 (m, 4H), 1.15 (s, 3H), 1.02 (s, 3H). | LCMS (ES, m/z): 588 [M + H]+, Rt 0.568 min. |
| 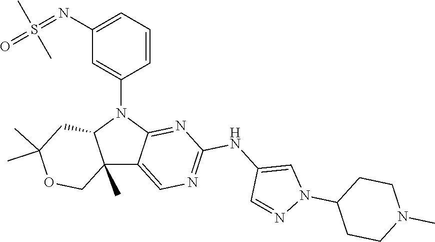 [SMILES: CN1CCC(n2cc(Nc3ncc4c(n3)N(c3cccc(N=S(C)(C)=O)c3)[C@H]3CC(C)(C)OC[C@@]43C)cn2)CC1] | 715, 716 | 1H NMR (300 MHz, DMSO-d6) δ 8.95 (s, 1H), 7.86 (s, 1H), 7.81 (s, 1H), 7.46 (s, 1H), 7.27-7.20 (m, 2H), 7.08 (s, 1H), 6.76 (d, J = 7.5 Hz, 1H), 4.30 (t, J = 5.4 Hz, 1H), 4.02- 3.95 (m, 1H), 3.71-3.58 (m, 1H), 3.57-3.49 (m, 1H), 3.23 (s, 6H), 2.89-2.82 (m, 2H), 2.20 (s, 3H), 2.10-1.96 (m, 2H), 1.96-1.75 (m, 5H), 1.74-1.59 (m, 1H), 1.31 (s, 3H), 1.14 (s, 3H), 1.02 (s, 3H). | LCMS (ES, m/z): 565 [M + H]+, Rt 0.546 min. |
| 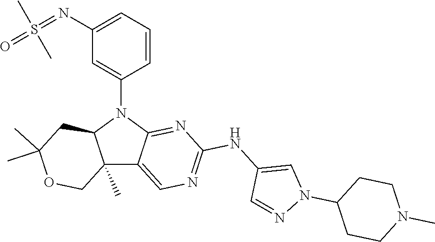 [SMILES: CN1CCC(n2cc(Nc3ncc4c(n3)N(c3cccc(N=S(C)(C)=O)c3)[C@@H]3CC(C)(C)OC[C@]43C)cn2)CC1] | | 1H NMR (300 MHz, DMSO-d6) δ 8.95 (s, 1H), 7.86 (s, 1H), 7.81 (s, 1H), 7.46 (s, 1H), 7.27-7.20 (m, 2H), 7.08 (s, 1H), 6.76 (d, J = 6.6 Hz, 1H), 4.30 (t, J = 5.4 Hz, 1H), 4.02- 3.95 (m, 1H), 3.71-3.58 (m, 1H), 3.57-3.49 (m, 1H), 3.23 (s, 6H), 2.88-2.79 (m, 2H), 2.21 (s, 3H), 2.11-1.99 (m, 2H), 1.97-1.74 (m, 5H), 1.73-1.55 (m, 1H), 1.31 (s, 3H), 1.14 (s, 3H), 1.02 (s, 3H). | LCMS (ES, m/z): 565 [M + H]+, Rt 0.550 min. |

### Examples 569 and 570. (4bR,8aR)-4b,7,7-trimethyl-9-((R)-7-methyl-7-(methylamino)-6,7-dihydro-5H-cyclopenta[b]pyridin-2-yl)-N-(4-morpholinophenyl)-4b,5,7,8,8a,9-hexahydropyrano[3′,4′:4,5]pyrrolo[2,3-d]pyrimidin-2-amine and (4bS,8aS)-4b,7,7-trimethyl-9-((R)-7-methyl-7-(methylamino)-6,7-dihydro-5H-cyclopenta[b]pyridin-2-yl)-N-(4-morpholinophenyl)-4b,5,7,8,8a,9-hexahydropyrano[3′,4′:4,5]pyrrolo[2,3-d]pyrimidin-2-amine

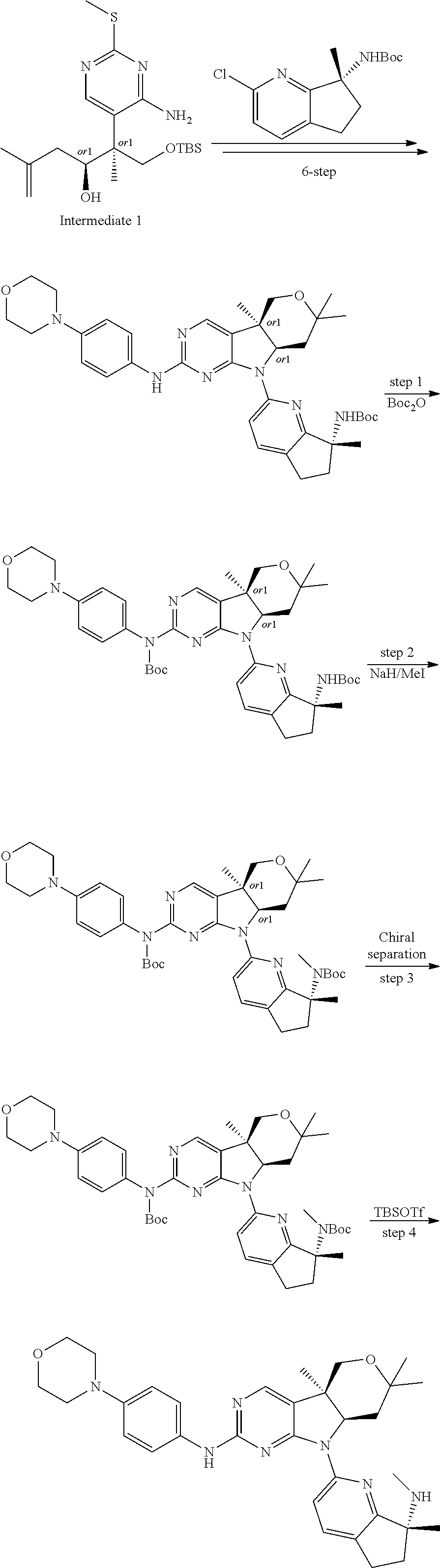

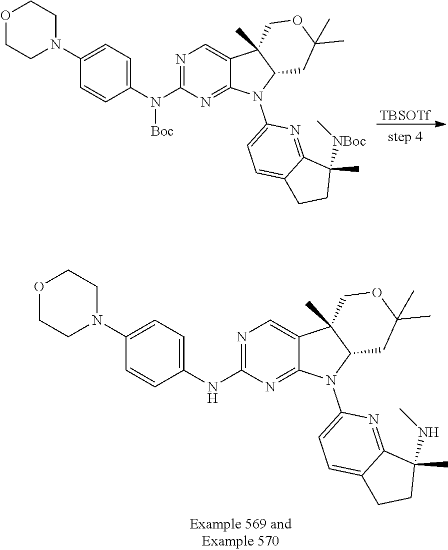 [SMILES: "TBSOTf" ; CN(C(=O)OC(C)(C)C)[C@]1(C)CCc2ccc(N3c4nc(N(C(=O)OC(C)(C)C)c5ccc(N6CCOCC6)cc5)ncc4[C@]4(C)COC(C)(C)C[C@H]34)nc21 ; CN[C@]1(C)CCc2ccc(N3c4nc(Nc5ccc(N6CCOCC6)cc5)ncc4[C@]4(C)COC(C)(C)C[C@H]34)nc21]

### Step 1. tert-butyl rel-((4bR,8aR)-9-((R)-7-((tert-butoxycarbonyl)amino)-7-methyl-6,7-dihydro-5H-cyclopenta[b]pyridin-2-yl)-4b,7,7-trimethyl-4b,5,7,8,8a,9-hexahydropyrano[3′,4′:4,5]pyrrolo[2,3-d]pyrimidin-2-yl)(4-morpholinophenyl)carbamate

Following the experimental procedures described in Examples 1 and 2/steps 1-6, tert-butyl ((R)-7-methyl-2-rel-((4bR,8aR)-4b,7,7-trimethyl-2-((4-morpholinophenyl)amino)-4b,7,8,8a-tetrahydropyrano[3′,4′:4,5]pyrrolo[2,3-d]pyrimidin-9(5H)-yl)-6,7-dihydro-5H-cyclopenta[b]pyridin-7-yl)carbamate was prepared stating from rel-(2S,3S)-2-(4-amino-2-(methylthio)pyrimidin-5-yl)-1-((tert-butyldimethylsilyl)oxy)-2,5-dimethylhex-5-en-3-ol (Intermediate 1) and tert-butyl (R)-(2-chloro-7-methyl-6,7-dihydro-5H-cyclopenta[b]pyridin-7-yl)carbamate (Intermediate 181/182) which was used directly in the next step described below.

To a stirred solution of tert-butyl ((R)-7-methyl-2-rel-((4bR,8aR)-4b,7,7-trimethyl-2-((4-morpholinophenyl)amino)-4b,7,8,8a-tetrahydropyrano[3′,4′:4,5]pyrrolo[2,3-d]pyrimidin-9(5H)-yl)-6,7-dihydro-5H-cyclopenta[b]pyridin-7-yl)carbamate (130 mg, 202.55 umol, 1 eq.) in THF (5 mL) was added DMAP (24.75 mg, 202.55 umol, 1 eq.) and pyridine (32.04 mg, 405.11 umol, 2 eq.) at room temperature. The resulting mixture was stirred for 15 min at room temperature. To the above mixture was added Boc₂O (88.41 mg, 405.11 umol, 2 eq.) at 0° C. The reaction was stirred for 16 h at 70° C. and was allowed to cool down to room temperature. The reaction mixture was concentrated under reduced pressure. The residue was purified by silica gel column chromatography (eluting with 3:2 EA/PE) to afford tert-butyl rel-((4bR,8aR)-9-((R)-7-((tert-butoxycarbonyl)amino)-7-methyl-6,7-dihydro-5H-cyclopenta[b]pyridin-2-yl)-4b,7,7-trimethyl-4b,5,7,8,8a,9-hexahydropyrano[3′,4′:4,5]pyrrolo[2,3-d]pyrimidin-2-yl)(4-morpholinophenyl)carbamate (130 mg, 175.44 umol, 87% yield) as a yellow solid. LCMS (ES, m/z): 742 [M+H]⁺, Rt 0.819 min.

### Step 2. tert-butyl ((R)-2-rel-((4bR,8aR)-2-((tert-butoxycarbonyl)(4-morpholinophenyl)amino)-4b,7,7-trimethyl-4b,7,8,8a-tetrahydropyrano[3′,4′:4,5]pyrrolo[2,3-d]pyrimidin-9(5H)-yl)-7-methyl-6,7-dihydro-5H-cyclopenta[b]pyridin-7-yl)(methyl)carbamate

To a stirred solution of tert-butyl rel-((4bR,8aR)-9-((R)-7-((tert-butoxycarbonyl)amino)-7-methyl-6,7-dihydro-5H-cyclopenta[b]pyridin-2-yl)-4b,7,7-trimethyl-4b,5,7,8,8a,9-hexahydropyrano[3′,4′:4,5]pyrrolo[2,3-d]pyrimidin-2-yl)(4-morpholinophenyl)carbamate (130 mg, 175.22 umol, 1 eq.) in DMF (6 mL) was added NaH (8.41 mg, 350.44 umol, 2 eq.) at 0° C. under N₂ atmosphere. The reaction was stirred for 1 h at 0° C. under N₂ atmosphere before Mel (37.31 mg, 262.83 umol, 1.5 eq.) was added at 0° C. under the N₂ atmosphere. The resulting mixture was stirred for 3 h at room temperature and was concentrated under reduced pressure. The residue was purified by silica gel column chromatography (eluting with 3:2 EA/PE) to afford tert-butyl ((R)-2-rel-((4bR,8aR)-2-((tert-butoxycarbonyl)(4-morpholinophenyl)amino)-4b,7,7-trimethyl-4b,7,8,8a-tetrahydropyrano[3′,4′:4,5]pyrrolo[2,3-d]pyrimidin-9(5H)-yl)-7-methyl-6,7-dihydro-5H-cyclopenta[b]pyridin-7-yl)(methyl)carbamate (100 mg, 132.27 umol, 75% yield) as a yellow solid. LCMS (ES, m/z): 756 [M+H]⁺, Rt 0.837 min.

### Step 3. tert-butyl ((R)-2-((4bR,8aR)-2-((tert-butoxycarbonyl)(4-morpholinophenyl)amino)-4b,7,7-trimethyl-4b,7,8,8a-tetrahydropyrano[3′,4′:4,5]pyrrolo[2,3-d]pyrimidin-9(5H)-yl)-7-methyl-6,7-dihydro-5H-cyclopenta[b]pyridin-7-yl)(methyl)carbamate and tert-butyl ((R)-2-((4bS,8aS)-2-((tert-butoxycarbonyl)(4-morpholinophenyl)amino)-4b,7,7-trimethyl-4b,7,8,8a-tetrahydropyrano[3′,4′:4,5]pyrrolo[2,3-d]pyrimidin-9(5H)-yl)-7-methyl-6,7-dihydro-5H-cyclopenta[b]pyridin-7-yl)(methyl)carbamate

Racemic tert-butyl ((R)-2-rel-((4bR,8aR)-2-((tert-butoxycarbonyl)(4-morpholinophenyl)amino)-4b,7,7-trimethyl-4b,7,8,8a-tetrahydropyrano[3′,4′:4,5]pyrrolo[2,3-d]pyrimidin-9(5H)-yl)-7-methyl-6,7-dihydro-5H-cyclopenta[b]pyridin-7-yl)(methyl)carbamate (100 mg, 132.27 umol, 1 eq.) was separated by chiral HPLC (Column: CHIRALPAK IE, 2×25 cm/5 μm; Mobile Phase A: Hex:DCM=5:1 (0.5% 2M NH₃-MeOH), Mobile Phase B: IPA (30% B in 12.75 min); Flow rate: 20 mL/min; 220/254 nm; RT1(min): 6.816; RT2(min): 10.24) to afford tert-butyl ((R)-2-((4bR,8aR)-2-((tert-butoxycarbonyl)(4-morpholinophenyl)amino)-4b,7,7-trimethyl-4b,7,8,8a-tetrahydropyrano[3′,4′:4,5]pyrrolo[2,3-d]pyrimidin-9(5H)-yl)-7-methyl-6,7-dihydro-5H-cyclopenta[b]pyridin-7-yl)(methyl)carbamate (30 mg, 30% yield) as a yellow solid and

tert-butyl ((R)-2-((4bS,8aS)-2-((tert-butoxycarbonyl)(4-morpholinophenyl)amino)-4b,7,7-trimethyl-4b,7,8,8a-tetrahydropyrano[3′,4′:4,5]pyrrolo[2,3-d]pyrimidin-9(5H)-yl)-7-methyl-6,7-dihydro-5H-cyclopenta[b]pyridin-7-yl)(methyl)carbamate (35 mg, 35% yield) as a yellow solid. LCMS (ES, m/z): 756 [M+H]⁺, Rt 0.837 min. Absolute stereochemistry of title compounds was arbitrarily assigned upon chiral separation.

### Step 4. (4bR,8aR)-4b,7,7-trimethyl-9-((R)-7-methyl-7-(methylamino)-6,7-dihydro-5H-cyclopenta[b]pyridin-2-yl)-N-(4-morpholinophenyl)-4b,5,7,8,8a,9-hexahydropyrano[3′,4′:4,5]pyrrolo[2,3-d]pyrimidin-2-amine and (4bS,8aS)-4b,7,7-trimethyl-9-((R)-7-methyl-7-(methylamino)-6,7-dihydro-5H-cyclopenta[b]pyridin-2-yl)-N-(4-morpholinophenyl)-4b,5,7,8,8a,9-hexahydropyrano[3′,4′:4,5]pyrrolo[2,3-d]pyrimidin-2-amine

To a stirred mixture of tert-butyl ((R)-2-((4bR,8aR)-2-((tert-butoxycarbonyl)(4-morpholinophenyl)amino)-4b,7,7-trimethyl-4b,7,8,8a-tetrahydropyrano[3′,4′:4,5]pyrrolo[2,3-d]pyrimidin-9(5H)-yl)-7-methyl-6,7-dihydro-5H-cyclopenta[b]pyridin-7-yl)(methyl)carbamate (30 mg, 39.68 umol, 1 eq.) in DCM (2 mL) was added TBSOTf (104.91 mg, 396.85 umol, 10 eq.) dropwise under 0° C. The reaction was stirred for 2 h at 0° C. and then was concentrated under reduced pressure. The crude product was purified by Prep-HPLC (Column: XBridge Prep Phenyl OBD Column, 19×250 mm/5 μm; Mobile Phase A: Water (10 mmol/L NH₄HCO₃+0.1% NH₃·H₂O), Mobile Phase B: ACN (55% to 85% B in 7 min); Flow rate: 25 mL/min; Wave Length: 254 nm; RT1(min): 5.08) to afford (4bR,8aR)-4b,7,7-trimethyl-9-((R)-7-methyl-7-(methylamino)-6,7-dihydro-5H-cyclopenta[b]pyridin-2-yl)-N-(4-morpholinophenyl)-4b,5,7,8,8a,9-hexahydropyrano[3′,4′:4,5]pyrrolo[2,3-d]pyrimidin-2-amine (17.2 mg, 78% yield) as a white solid. ¹HNMR (300 MHz, DMSO-d6) δ 9.09 (s, 1H), 8.46 (d, J=8.7 Hz, 1H), 7.96 (s, 1H), 7.66-7.57 (m, 3H), 6.91 (d, J=8.7 Hz, 2H), 4.70-4.66 (m, 1H), 3.76-3.64 (m, 6H), 3.07-3.04 (m, 4H), 2.94-2.84 (m, 1H), 2.79-2.69 (m, 1H), 2.29-2.09 (m, 5H), 1.99-1.90 (m, 1H), 1.69-1.61 (m, 1H), 1.35 (s, 3H), 1.30 (s, 3H), 1.23 (s, 3H), 1.02 (s, 3H). LCMS (ES, m/z): 556 [M+H]⁺, Rt 0.642 min.

To a stirred mixture of tert-butyl ((R)-2-((4bS,8aS)-2-((tert-butoxycarbonyl)(4-morpholinophenyl)amino)-4b,7,7-trimethyl-4b,7,8,8a-tetrahydropyrano[3′,4′:4,5]pyrrolo[2,3-d]pyrimidin-9(5H)-yl)-7-methyl-6,7-dihydro-5H-cyclopenta[b]pyridin-7-yl)(methyl)carbamate (35 mg, 46.30 umol, 1 eq.) in DCM (2 mL) was added TBSOTf (104.90 mg, 396.86 umol, 10 eq.) dropwise under 0° C. The resulting mixture was stirred for 2 h at 0° C. then was concentrated under reduced pressure. The crude product was purified by Prep-HPLC (Column: XBridge Shield RP18 OBD Column, 30×150 mm/5 μm; Mobile Phase A: Water (10 mmol/L NH₄HCO₃+0.1% NH₃·H₂O), Mobile Phase B: ACN (47% to 55% B in 10 min); Flow rate: 60 mL/min; Wave Length: 254 nm; RT1(min): 6.5) to afford (4bS,8aS)-4b,7,7-trimethyl-9-((R)-7-methyl-7-(methylamino)-6,7-dihydro-5H-cyclopenta[b]pyridin-2-yl)-N-(4-morpholinophenyl)-4b,5,7,8,8a,9-hexahydropyrano[3′,4′:4,5]pyrrolo[2,3-d]pyrimidin-2-amine (13.3 mg, 51% yield) as a white solid. ¹HNMR (300 MHz, DMSO-d6) δ 9.09 (s, 1H), 8.45 (d, J=8.7 Hz, 1H), 7.97 (s, 1H), 7.68-7.57 (m, 3H), 6.91 (d, J=8.4 Hz, 2H), 4.70-4.66 (m, 1H), 3.77-3.74 (m, 5H), 3.67-3.63 (m, 1H), 3.05 (t, J=4.8 Hz, 4H), 2.92-2.75 (m, 2H), 2.24-2.15 (m, 5H), 2.00-1.91 (m, 1H), 1.73-1.66 (m, 1H), 1.36 (s, 3H), 1.30 (s, 3H), 1.22 (s, 3H), 1.01 (s, 3H). LCMS (ES, m/z): 556 [M+H]⁺, Rt 0.614 min.

TABLE 17
The examples in the following table were synthesized using appropriate
intermediates and reaction conditions as demonstrated for Examples 1-4, 111-112, 234-235, 711-
712, 569-570. Absolute stereochemistry of the examples in the following table was arbitrarily
assigned.

| Structure | Ex # | ¹H NMR | LCMS |
|---|---|---|---|
| 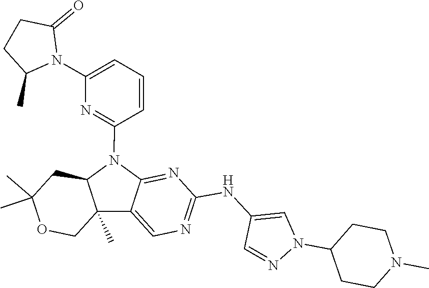 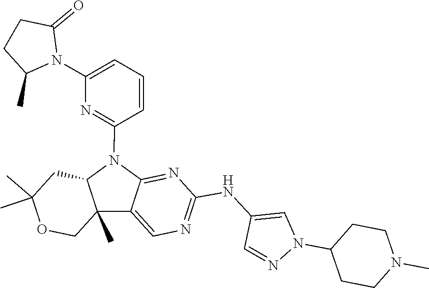 | 555, 556 | 1H NMR (400 MHz, DMSO-d6) δ 9.17 (s, 1H), 8.37 (s, 1H), 8.00 (s, 1H), 7.92-7.90 (m, 2H), 7.79-7.75 (m, 1H), 7.47 (s, 1H), 4.72-4.67 (m, 1H), 4.51-4.47 (m, 1H), 4.09-4.01 (m, 1H), 3.81-3.78 (m, 1H), 3.69-3.66 (m, 1H), 2.87-2.83 (m, 2H), 2.80-2.67 (m, 1H), 2.47-2.42 (m, 1H), 2.34-2.28 (m, 2H), 2.23 (s, 3H), 2.08- 1.85 (m, 6H), 1.77-1.70 (m, 1H), 1.59- 1.54 (m, 1H), 1.41-1.39 (m, 3H), 1.28 (s, 3H), 1.25 (s, 3H), 1.07 (s, 3H). 1H NMR (400 MHz, DMSO-d6) δ 9.17 (s, 1H), 8.35 (s, 1H), 8.01 (s, 1H), 7.91 (s, 1H), 7.86-7.84 (m, 1H), 7.80-7.76 (m, 1H), 7.47 (s, 1H), 4.86-4.81 (m, 1H), 4.64-4.61 (m, 1H), 4.05-4.00 (m, 1H), 3.84-3.81 (m, 1H), 3.68-3.65 (m, 1H), 2.87-2.77 (m, 3H), 2.47-2.41 (m, 1H), 2.34-2.26 (m, 1H), 2.21-2.15 (m, 4H), 2.08-2.01 (m, 2H), 1.97-1.88 (m, 4H), 1.74-1.73 (m, 1H), 1.58-1.52 (m, 1H), 1.31-1.29 (m, 3H), 1.25 (s, 3H), 1.24 (s, 3H), 1.05 (s, 3H). | LCMS (ES, m/z): 572.30 [M + H]+, Rt 0.683 min. LCMS (ES, m/z): 572.30 [M + H]+, Rt 0.675 min. |
| 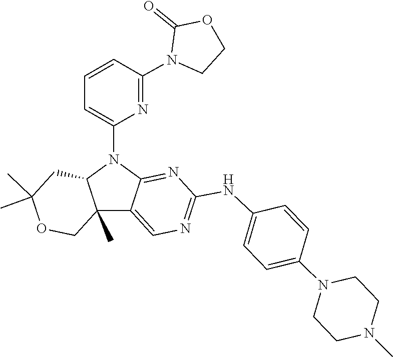 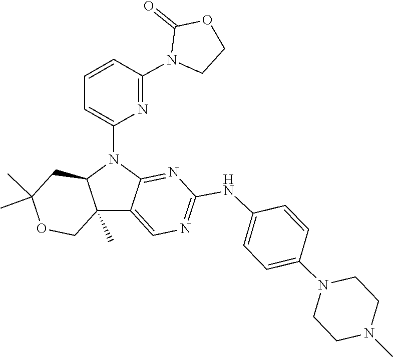 | 576, 577 | 1H NMR (300 MHz, DMSO-d6) δ 9.12 (s, 1H), 8.41 (d, J = 8.4 Hz, 1H), 8.00 (s, 1H), 7.83 (t, J = 8.1 Hz, 1H), 7.69 (d, J = 8.1 Hz, 1H), 7.55 (d, J = 9.0 Hz, 2H), 6.91 (d, J = 9.0 Hz, 2H), 4.62-4.57 (m, 1H), 4.48 (t, J = 8.1 Hz, 2H), 4.33-4.17 (m, 2H), 3.81-3.77 (m, 1H), 3.69-3.65 (m, 1H), 3.10-3.07 (m, 4H), 2.47 (d, J = 6.0 Hz, 4H), 2.32-2.27 (m, 1H), 2.24 (s, 3H), 1.63-1.55 (m, 1H), 1.29 (s, 3H), 1.26 (s, 3H), 1.06 (s, 3H). 1H NMR (300 MHz, DMSO-d6) δ 9.13 (s, 1H), 8.41 (d, J = 8.1 Hz, 1H), 8.00 (s, 1H), 7.83 (t, J = 8.4 Hz, 1H), 7.69 (d, J = 7.8 Hz, 1H), 7.55 (d, J = 8.7 Hz, 2H), 6.95-6.89 (m, 2H), 4.62-4.57 (m, 1H), 4.48 (t, J = 8.1 Hz, 2H), 4.33-4.14 (m, 2H), 3.81-3.77 (m, 1H), 3.69-3.65 (m, 1H), 3.10-3.06 (m, 4H), 2.47 (d, J = 6.0 Hz, 4H), 2.32-2.23 (m, 4H), 1.63-1.55 (m, 1H), 1.29 (s, 3H), 1.26 (s, 3H), 1.06 (s, 3H). | LCMS (ES, m/z): 571 [M + H]+, Rt 0.640 min. LCMS (ES, m/z): 571 [M + H]+, Rt 0.648 min. |
| 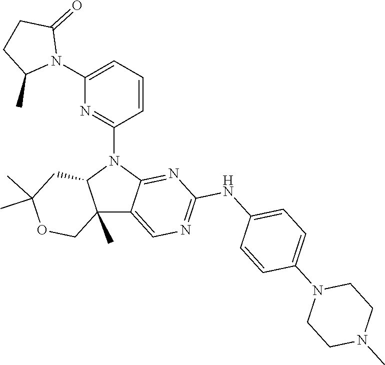 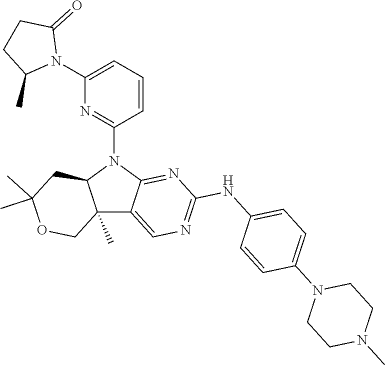 | 582, 583 | 1H NMR (400 MHz, DMSO-d6) δ 9.13 (s, 1H), 8.41(d, J = 8.0 Hz, 1H), 8.00 (s, 1H), 7.92 (d, J = 7.6 Hz, 1H), 7.79 (t, J = 8.0 Hz, 1H), 7.56 (d, J = 9.2 Hz, 2H), 6.91 (d, J = 9.2 Hz, 2H), 4.71-4.67 (m, 1H), 4.52- 4.48 (m, 1H), 3.87-3.79 (m, 1H), 3.72- 3.67 (m, 1H), 3.09-3.06 (m, 4H), 2.83- 2.74 (m, 1H), 2.48-2.42 (m, 5H), 2.34- 2.28 (m, 2H), 2.23 (s, 3H), 1.77-1.71 (m, 1H), 1.60-1.54 (m, 1H), 1.41-1.40 (m, 3H), 1.28 (s, 3H), 1.26 (s, 3H), 1.08 (s, 3H). 1H NMR (400 MHz, DMSO-d6) δ 9.13 (s, 1H), 8.42 (d, J = 7.6 Hz, 1H), 8.01 (s, 1H), 7.85-7.77 (m, 2H), 7.56 (d, J = 8.8 Hz, 2H), 6.91 (d, J = 9.2 Hz, 2H), 4.87- 4.83 (m, 1H), 4.66-4.62 (m, 1H), 3.89- 3.81 (m, 1H), 3.73-3.64 (m, 1H), 3.08- 3.06 (m, 4H), 2.84-2.75 (m, 1H), 2.47- 2.45 (m, 5H), 2.29-2.26 (m, 1H), 2.23- 2.15 (m, 4H), 1.76-1.71 (m, 1H), 1.58- 1.53 (m, 1H), 1.30 (d, J = 6.4 Hz, 3H), 1.26 (s, 3H), 1.25 (s, 3H), 1.06 (s, 3H). | LCMS (ES, m/z): 583 [M + H]+. Rt 0.692 min. LCMS (ES, m/z): 583 [M + H]+. Rt 0.693 min. |
| 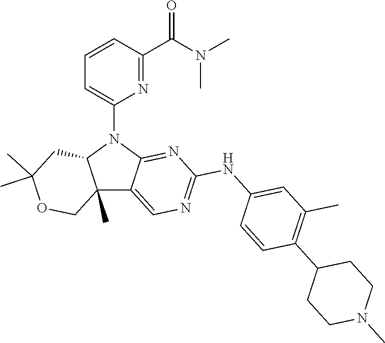 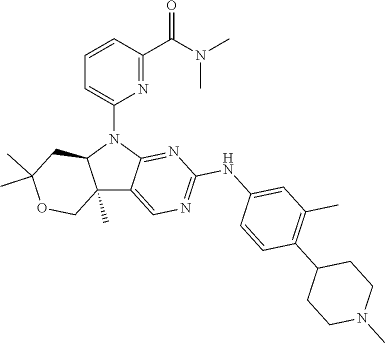 | 584, 585 | 1H NMR (300 MHz, DMSO-d6) δ 9.26 (s, 1H), 8.70 (d, J = 8.4 Hz, 1H), 8.08 (s, 1H), 7.95-7.84 (m, 1H), 7.54 (d, J = 2.1 Hz, 1H), 7.48-7.39 (m, 1H), 7.22 (d, J = 7.5 Hz, 1H), 7.13 (d, J = 8.4 Hz, 1H), 4.69-4.59 (m, 1H), 3.84-3.61 (m, 2H), 3.03 (s, 6H), 2.93-2.84 (m, 2H), 2.65- 2.54 (m, 1H), 2.29 (s, 3H), 2.21 (s, 3H), 2.17-2.07 (m, 1H), 2.04-1.94 (m, 2H), 1.74-1.60 (m, 5H), 1.28 (s, 3H), 1.20 (s, 3H), 1.01 (s, 3H) 1H NMR (300 MHz, DMSO-d6) δ 9.26 (s, 1H), 8.70 (d, J = 8.7 Hz, 1H), 8.08 (s, 1H), 7.95-7.84 (m, 1H), 7.54 (d, J = 2.4 Hz, 1H), 7.48-7.39 (m, 1H), 7.22 (d, J = 7.5 Hz, 1H), 7.13 (d, J = 8.4 Hz, 1H), 4.69-4.59 (m, 1H), 3.84-3.61 (m, 2H), 3.03 (s, 6H), 2.94-2.85 (m, 2H), 2.65- 2.54 (m, 1H), 2.29 (s, 3H), 2.21 (s, 3H), 2.17-2.07 (m, 1H), 2.06-1.92 (m, 2H), 1.74-1.60 (m, 5H), 1.28 (s, 3H), 1.20 (s, 3H), 1.01 (s, 3H). | LCMS (ES, m/z): 570.35 [M + H]+, Rt 0.633 min. LCMS (ES, m/z): 570.35 [M + H]+, Rt 0.633 min. |
| 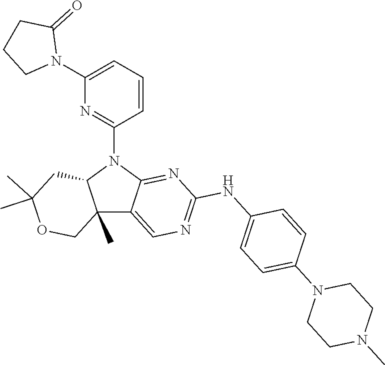 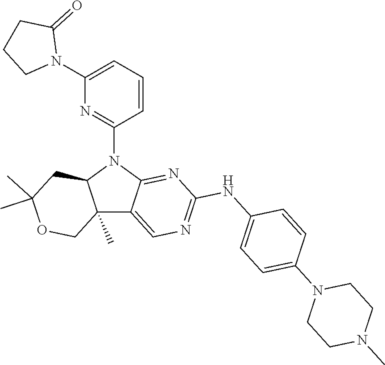 | 586, 587 | 1H NMR (300 MHz, DMSO-d6) δ 9.12 (s, 1H), 8.40 (d, J = 8.1 Hz, 1H), 7.99 (s, 1H), 7.96-7.88 (m, 1H), 7.85-7.73 (m, 1H), 7.55 (d, J = 8.7 Hz, 2H), 6.90 (d, J = 9.0 Hz, 2H), 4.64-4.51 (m, 1H), 4.18- 3.95 (m, 2H), 3.83-3.61 (m, 2H), 3.12- 3.02 (m, 4H), 2.64-2.53 (m, 2H), 2.51- 2.42 (m, 4H), 2.35-2.23 (m, 4H), 2.14- 2.01 (m, 2H), 1.65-1.52 (m, 1H), 1.28 (s, 3H), 1.25 (s, 3H), 1.06 (s, 3H). 1H NMR (300 MHz, DMSO-d6) δ 9.12 (s, 1H), 8.40 (d, J = 8.1 Hz, 1H), 7.99 (s, 1H), 7.96-7.88 (m, 1H), 7.85-7.73 (m, 1H), 7.55 (d, J = 8.7 Hz, 2H), 6.90 (d, J = 9.0 Hz, 2H), 4.64-4.51 (m, 1H), 4.18- 3.95 (m, 2H), 3.83-3.61 (m, 2H), 3.12- 3.02 (m, 4H), 2.64-2.53 (m, 2H), 2.51- 2.42 (m, 4H), 2.35-2.23 (m, 4H), 2.14- 2.01 (m, 2H), 1.65-1.52 (m, 1H), 1.28 (s, 3H), 1.25 (s, 3H), 1.06 (s, 3H). | LCMS(ES, m/z): 569 [M + H]+. Rt 0.671 min. LCMS(ES, m/z): 569 [M + H]+. Rt 0.672 min. |
| 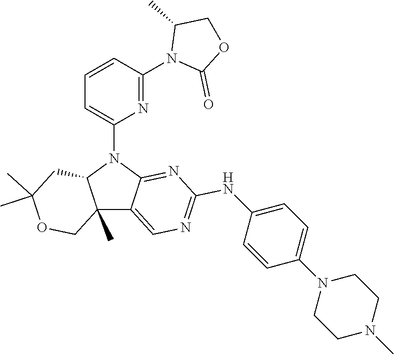 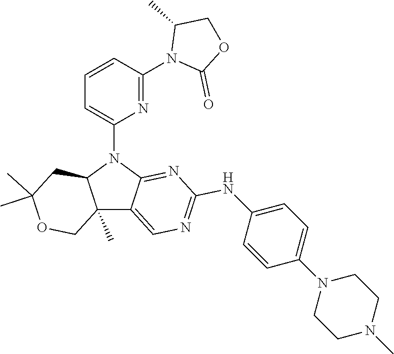 | 588, 589 | 1H NMR (300 MHz, DMSO-d6) δ 9.14 (s, 1H), 8.42 (d, J = 8.1 Hz, 1H), 8.03 (s, 1H), 7.83 (t, J = 8.1 Hz, 1H), 7.65 (d, J = 7.8 Hz, 1H), 7.55 (d, J = 9.0 Hz, 2H), 6.91 (d, J = 9.0 Hz, 2H), 4.96-4.91 (m, 1H), 4.69-4.65 (m, 1H), 4.55 (t, J = 8.4 Hz, 1H), 4.15-4.11 (m, 1H), 3.85-3.69 (m, 2H), 3.09-3.06 (m, 4H), 2.51-2.42 (m, 4H), 2.28-2.18 (m, 4H), 1.62-1.48 (m, 1H), 1.39 (d, J = 6.3 Hz, 3H), 1.25 (d, J = 3.3 Hz, 6H), 1.07 (s, 3H). 1H NMR (300 MHz, DMSO-d6) δ 9.14 (s, 1H), 8.42 (d, J = 8.1 Hz, 1H), 8.01 (s, 1H), 7.83 (t, J = 8.1 Hz, 1H), 7.66 (d, J = 8.1 Hz, 1H), 7.55 (d, J = 8.7 Hz, 2H), 6.91 (d, J = 9.0 Hz, 2H), 4.83-4.77 (m, 1H), 4.60-4.46 (m, 2H), 4.13-4.09 (m, 1H), 3.87-3.62 (m, 2H), 3.12-3.01 (m, 4H), 2.51-2.42 (m, 4H), 2.39-2.32 (m, 1H), 2.23 (s, 3H), 1.63-1.48 (m, 4H), 1.28 (d, J = 8.7 Hz, 6H), 1.08 (s, 3H). | LCMS (ES, m/z): 585.35 [M + H]+, Rt 0.733 min. LCMS (ES, m/z): 585.30 [M + H]+, Rt 0.742 min. |
| 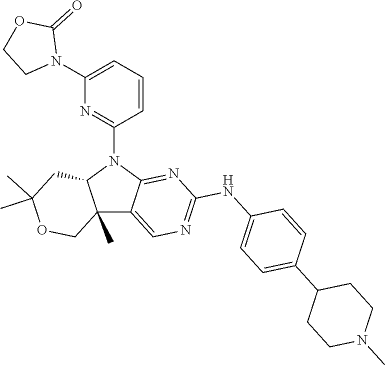 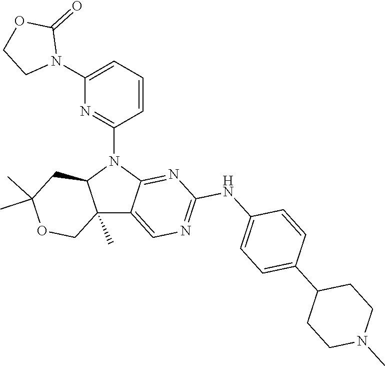 | 590, 591 | 1H NMR (300 MHz, DMSO-d6) δ 9.31 (s, 1H), 8.42 (d, J = 8.1 Hz, 1H), 8.04 (s, 1H), 7.84 (t, J = 8.1 Hz, 1H), 7.73-7.61 (m, 3H), 7.17 (d, J = 8.4 Hz, 2H), 4.63-4.55 (m, 1H), 4.53-4.41 (m, 2H), 4.33-4.15 (m, 2H), 3.86-3.62 (m, 2H), 2.94-2.86 (m, 2H), 2.49-2.24 (m, 2H), 2.22 (s, 3H), 2.09-1.92 (m, 2H), 1.80-1.57 (m, 5H), 1.30 (s, 3H), 1.26 (s, 3H), 1.06 (s, 3H). 1H NMR (300 MHz, DMSO-d6) δ 9.31 (s, 1H), 8.42 (d, J = 8.1 Hz, 1H), 8.04 (s, 1H), 7.83 (t, J = 8.1 Hz, 1H), 7.73-7.61 (m, 3H), 7.17 (d, J = 8.4 Hz, 2H), 4.63-4.55 (m, 1H), 4.53-4.41 (m, 2H), 4.33-4.15 (m, 2H), 3.86-3.62 (m, 2H), 2.94-2.86 (m, 2H), 2.49-2.24 (m, 2H), 2.23 (s, 3H), 2.09-1.92 (m, 2H), 1.80-1.57 (m, 5H), 1.30 (s, 3H), 1.26 (s, 3H), 1.06 (s, 3H). | LCMS (ES, m/z): 570 [M + H]+. Rt 0.671 min. LCMS (ES, m/z): 570 [M + H]+. Rt 0.666 min. |
| 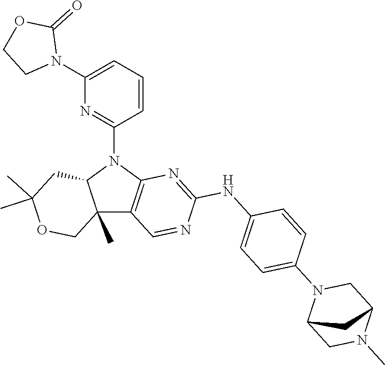 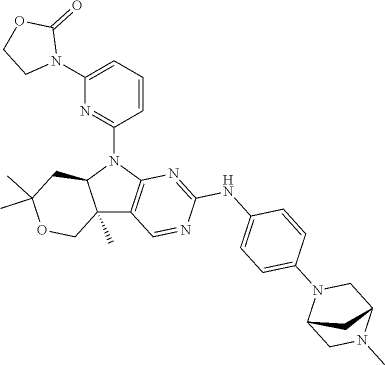 | 592, 593 | 1H NMR (300 MHz, DMSO-d6) δ 8.98 (s, 1H), 8.40 (d, J = 8.1 Hz, 1H), 7.97 (s, 1H), 7.79 (t, J = 7.8 Hz, 1H), 7.68 (d, J = 7.8 Hz, 1H), 7.44 (d, J = 8.7 Hz, 2H), 6.55 (d, J = 8.7 Hz, 2H), 4.60-4.56 (m, 1H), 4.51-4.45 (m, 2H), 4.32-4.13 (m, 3H), 3.80-3.72 (m, 1H), 3.68-3.64 (m, 1H), 3.42-3.34 (m, 1H), 3.32-3.28 (m, 1H), 3.14-3.11 (m, 1H), 2.79-2.76 (m, 1H), 2.55-2.50 (m, 1H), 2.31-2.27 (m, 4H), 1.88-1.85 (m, 1H), 1.79-1.75 (m, 1H), 1.61-1.53 (m, 1H), 1.28 (s, 3H), 1.25 (s, 3H), 1.06 (s, 3H). 1H NMR (300 MHz, DMSO-d6) δ 8.98 (s, 1H), 8.40 (d, J = 8.1 Hz, 1H), 7.97 (s, 1H), 7.79 (t, J = 7.8 Hz, 1H), 7.68 (d, J = 7.8 Hz, 1H), 7.44 (d, J = 8.7 Hz, 2H), 6.55 (d, J = 8.7 Hz, 2H), 4.60-4.56 (m, 1H), 4.51-4.45 (m, 2H), 4.32-4.13 (m, 3H), 3.80-3.72 (m, 1H), 3.68-3.64 (m, 1H), 3.42-3.34 (m, 1H), 3.32-3.28 (m, 1H), 3.14-3.11 (m, 1H), 2.79-2.76 (m, 1H), 2.55-2.50 (m, 1H), 2.31-2.27 (m, 4H), 1.88-1.85 (m, 1H), 1.79-1.75 (m, 1H), 1.61-1.53 (m, 1H), 1.28 (s, 3H), 1.25 (s, 3H), 1.06 (s, 3H). | LCMS (ES, m/z): 583 [M + H]+, Rt 2.841 min. LCMS (ES, m/z): 583 [M + H]+, Rt 2.834 min. |
| 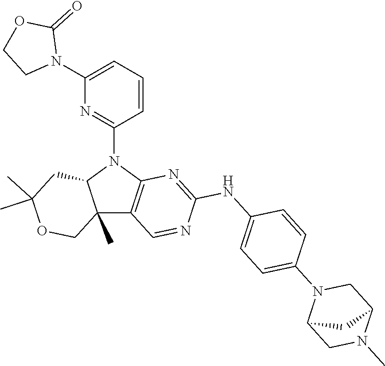 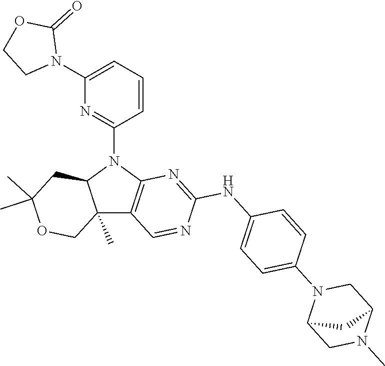 | 594, 595 | 1H NMR (300 MHz, DMSO-d6) δ 8.99 (s, 1H), 8.40 (d, J = 8.1 Hz, 1H), 7.97 (s, 1H), 7.78 (t, J = 8.1 Hz, 1H), 7.67 (d, J = 8.1 Hz, 1H), 7.44 (d, J = 8.4 Hz, 2H), 6.61-6.51 (m, 2H), 4.63-4.40 (m, 3H), 4.34-4.11 (m, 3H), 3.84-3.61 (m, 2H), 3.48-3.40 (m, 2H), 3.17-3.08 (m, 1H), 2.83-2.73 (m, 1H), 2.60-2.51 (m, 1H), 2.36-2.22 (m, 1H), 2.28 (s, 3H), 1.92- 1.74 (m, 2H), 1.63-1.50 (m, 1H), 1.27 (s, 3H), 1.25 (s, 3H), 1.06 (s, 3H). 1H NMR (300 MHz, DMSO-d6) δ 8.99 (s, 1H), 8.40 (d, J = 8.1 Hz, 1H), 7.97 (s, 1H), 7.79 (t, J = 8.1 Hz, 1H), 7.67 (d, J = 8.1 Hz, 1H), 7.44 (d, J = 8.4 Hz, 2H), 6.61-6.50 (m, 2H), 4.64-4.41 (m, 3H), 4.34-4.13 (m, 3H), 3.82-3.62 (m, 2H), 3.54-3.43 (m, 2H), 3.18-3.09 (m, 1H), 2.84-2.75 (m, 1H), 2.63-2.54 (m, 1H), 2.30 (s, 3H), 2.29-2.22 (m, 1H), 1.93- 1.76 (m, 2H), 1.64-1.50 (m, 1H), 1.27 (s, 3H), 1.25 (s, 3H), 1.06 (s, 3H). | LCMS (ES, m/z): 583 [M + H]+; RT: 1.227 min. LCMS (ES, m/z): 583 [M + H]+; RT: 0.639 min. |
| 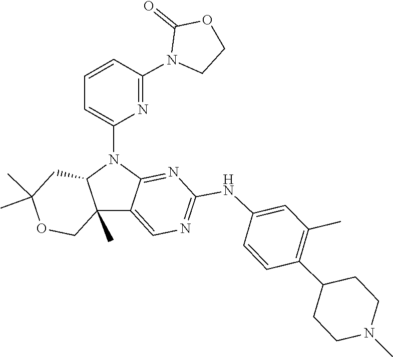 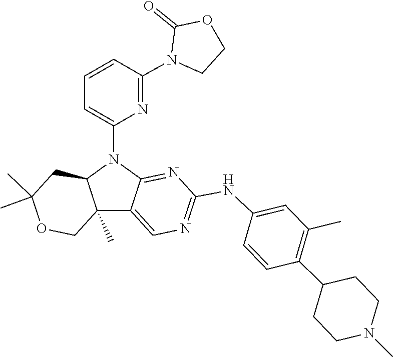 | 596, 597 | 1H NMR (300 MHz, DMSO-d6) δ 9.25 (s, 1H), 8.42 (d, J = 8.1 Hz, 1H), 8.04 (s, 1H), 7.84-7.79 (m, 1H), 7.71-7.68 (m, 1H), 7.56 (d, J = 2.1 Hz, 1H), 7.45-7.42 (m, 1H), 7.13-7.10 (m, 1H), 4.63-4.58 (m, 1H), 4.51-4.45 (m, 2H), 4.33-4.17 (m, 2H), 3.82-3.78 (m, 1H), 3.69-3.65 (m, 1H), 2.92-2.88 (m, 2H), 2.62-2.57 (m, 1H), 2.32-3.26 (m, 4H), 2.22 (s, 3H), 2.06-1.97 (m, 2H), 1.66-1.55 (m, 5H), 1.29 (s, 3H), 1.25 (s, 3H), 1.06 (s, 3H). 1H NMR (300 MHz, DMSO-d6) δ 9.24 (s, 1H), 8.42 (d, J = 8.1 Hz, 1H), 8.04 (s, 1H), 7.84-7.79 (m, 1H), 7.71-7.68 (m, 1H), 7.56 (d, J = 2.1 Hz, 1H), 7.45-7.42 (m, 1H), 7.13-7.10 (m, 1H), 4.63-4.58 (m, 1H), 4.51-4.45 (m, 2H), 4.29-4.17 (m, 2H), 3.82-3.78 (m, 1H), 3.69-3.65 (m, 1H), 2.90-2.86 (m, 2H), 2.61-2.56 (m, 1H), 2.32-3.26 (m, 4H), 2.21 (s, 3H), 2.03-1.95 (m, 2H), 1.65-1.55 (m, 5H), 1.29 (s, 3H), 1.25 (s, 3H), 1.06 (s, 3H). | LCMS (ES, m/z): 584 [M + H]+. Rt 0.649 min. LCMS (ES, m/z): 584 [M + H]+. Rt 0.656 min. |
| 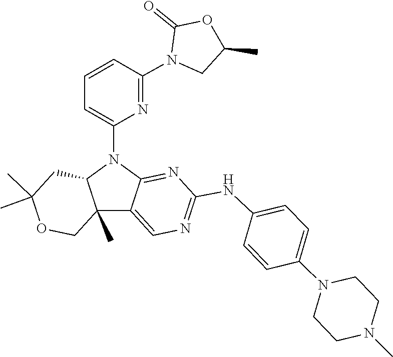 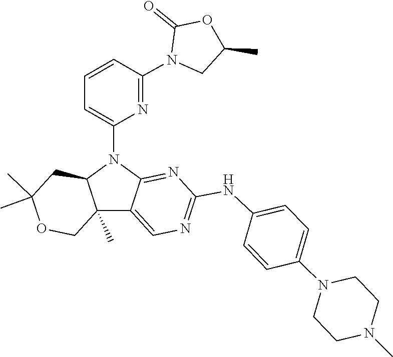 | 598, 599 | 1H NMR (300 MHz, DMSO-d6) δ 9.13 (s, 1H), 8.40 (d, J = 7.8 Hz, 1H), 7.99 (s, 1H), 7.82 (t, J = 8.1 Hz, 1H), 7.67 (d, J = 8.1 Hz, 1H), 7.55 (d, J = 9.0 Hz, 2H), 6.90 (d, J = 9.0 Hz, 2H), 4.89-4.80 (m, 1H), 4.61-4.57 (m, 1H), 4.42 (t, J = 9.0 Hz, 1H), 3.79-3.65 (m, 3H), 3.09-3.06 (m, 4H), 2.48-2.45 (m, 4H), 2.32-2.23 (m, 4H), 1.60-1.53 (m, 1H), 1.44 (d, J = 6.0 Hz, 3H), 1.29 (s, 3H), 1.25 (s, 3H), 1.07 (s, 3H). 1H NMR (300 MHz, DMSO-d6) δ 9.13 (s, 1H), 8.40 (d, J = 7.8 Hz, 1H), 8.00 (s, 1H), 7.82 (t, J = 8.1 Hz, 1H), 7.67 (d, J = 7.8 Hz, 1H), 7.55 (d, J = 9.0 Hz, 2H), 6.90 (d, J = 9.0 Hz, 2H), 4.89-4.82 (m, 1H), 4.61-4.57 (m, 1H), 4.30 (t, J = 9.0 Hz, 1H), 3.88-3.76 (m, 2H), 3.68-3.64 (m, 1H), 3.09-3.06 (m, 4H), 2.48-2.45 (m, 4H), 2.31-2.23 (m, 4H), 1.64-1.56 (m, 1H), 1.44 (d, J = 6.3 Hz, 3H), 1.29 (s, 3H), 1.25 (s, 3H), 1.06 (s, 3H). | LCMS (ES, m/z): 585.35 [M + H]+, Rt 0.642 min. LCMS (ES, m/z): 585.35 [M + H]+, Rt 0.642 min. |
| 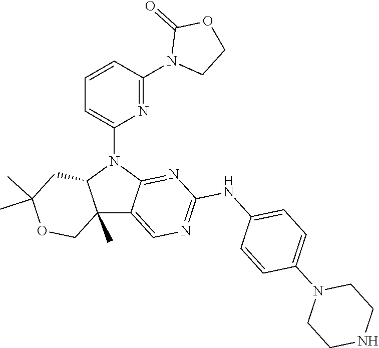 [SMILES: CC1(C)C[C@@H]2N(c3cccc(N4CCOC4=O)n3)c3nc(Nc4ccc(N5CCNCC5)cc4)ncc3[C@]2(C)CO1] 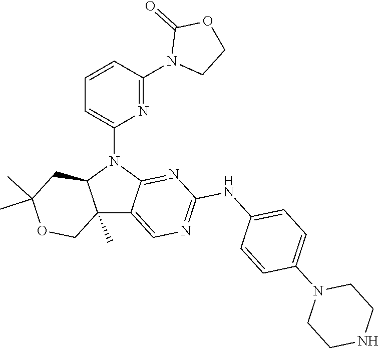 [SMILES: CC1(C)C[C@H]2N(c3cccc(N4CCOC4=O)n3)c3nc(Nc4ccc(N5CCNCC5)cc4)ncc3[C@@]2(C)CO1] | 600, 601 | 1H NMR (300 MHz, DMSO-d6) δ 9.13 (s, 1H), 8.41 (d, J = 8.1 Hz, 1H), 8.00 (s, 1H), 7.82 (t, J = 8.1 Hz, 1H), 7.68 (d, J = 8.1 Hz, 1H), 7.58-7.49 (m, 2H), 6.93- 6.84 (m, 2H), 4.64-4.53 (m, 1H), 4.53- 4.39 (m, 2H), 4.35-4.14 (m, 2H), 3.86- 3.61 (m, 2H), 3.15-2.93 (m, 4H), 2.93- 2.75 (m, 4H), 2.34-2.22 (m, 1H), 1.65- 1.51 (m, 1H), 1.28 (s, 3H), 1.25 (s, 3H), 1.06 (s, 3H). 1H NMR (300 MHz, DMSO-d6) δ 9.13 (s, 1H), 8.41 (d, J = 8.1 Hz, 1H), 8.00 (s, 1H), 7.82 (t, J = 8.1 Hz, 1H), 7.68 (d, J = 8.1 Hz, 1H), 7.58-7.49 (m, 2H), 6.93- 6.84 (m, 2H), 4.64-4.53 (m, 1H), 4.45- 4.39 (m, 2H), 4.35-4.14 (m, 2H), 3.86- 3.61 (m, 2H), 3.15-2.93 (m, 4H), 2.93- 2.75 (m, 4H), 2.34-2.22 (m, 1H), 1.65- 1.51 (m, 1H), 1.28 (s, 3H), 1.25 (s, 3H), 1.06 (s, 3H). | |
| 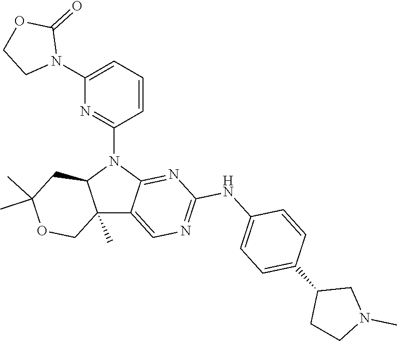 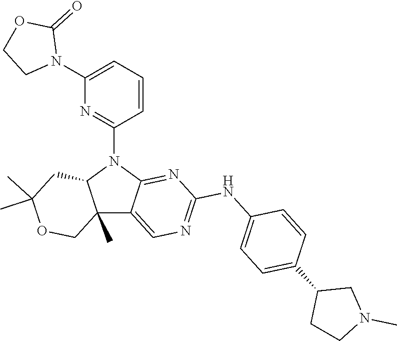 | 602, 603 | 1H NMR (300 MHz, DMSO-d6) δ 9.29 (s, 1H), 8.41 (d, J = 8.1 Hz, 1H), 8.03 (s, 1H), 7.84-7.79 (m, 1H), 7.71-7.62 (m, 3H), 7.19 (d, J = 8.4 Hz, 2H), 4.62-4.58 (m, 1H), 4.50-4.45 (m, 2H), 4.29-4.16 (m, 2H), 3.81-3.77 (m, 1H), 3.69-3.65 (m, 1H), 3.30-3.24 (m, 1H), 2.90-2.87 (m, 1H), 2.67-2.59 (m, 2H), 2.41-2.36 (m, 2H), 2.32 (s, 3H), 2.26-2.17 (m, 2 H), 1.81-1.70 (m, 1H), 1.63-1.58 (m, 1H), 1.29 (s, 3H), 1.25 (s, 3H), 1.05 (s, 3H). 1H NMR (300 MHz, DMSO-d6) δ 9.29 (s, 1H), 8.41 (d, J = 8.1 Hz, 1H), 8.03 (s, 1H), 7.80 (t, J = 8.4 Hz, 1H), 7.71-7.62 (m, 3H), 7.19 (d, J = 8.4 Hz, 2H), 4.62- 4.58 (m, 1H), 4.50-4.45 (m, 2H), 4.29- 4.16 (m, 2H), 3.81-3.77 (m, 1H), 3.69- 3.65 (m, 1H), 3.30-3.24 (m, 1H), 2.90- 2.87 (m, 1H), 2.67-2.59 (m, 2H), 2.41- 2.36 (m, 1H), 2.32 (s, 3H), 2.26-2.17 (m, 2 H), 1.81-1.70 (m, 1H), 1.63-1.58 (m, 1H), 1.29 (s, 3H), 1.25 (s, 3H), 1.05 (s, 3H). | LCMS (ES, m/z): 556 [M + H]+, Rt 0.630 min. LCMS (ES, m/z): 556 [M + H]+, Rt 0.656 min. |
| 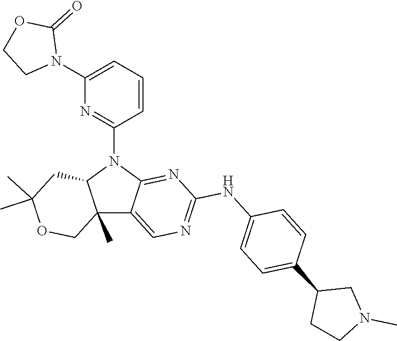 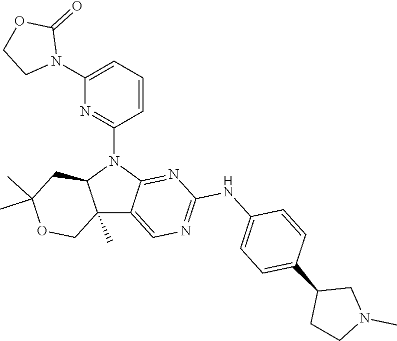 | 604, 605 | 1H NMR (400 MHz, DMSO-d6) δ 9.30 (s, 1H), 8.42 (d, J = 8.1 Hz, 1H), 8.04 (s, 1H), 7.82 (t, J = 8.1 Hz, 1H), 7.70 (d, J = 8.0 Hz, 1H), 7.63 (d, J = 8.1 Hz, 2H), 7.18 (d, J = 8.1 Hz, 2H), 4.62-4.59 (m, 1H), 4.50-4.45 (m, 2H), 4.29-4.17 (m, 2H), 3.85-3.77 (m, 1H), 3.73-3.62 (m, 1H), 3.29-3.24 (m, 1H), 2.87 (t, J = 8.4 Hz, 1H), 2.66-2.58 (m, 2H), 2.40-2.36 (m, 1H), 2.30 (s, 3H), 2.28-2.23 (m, 2H), 1.82-1.69 (m, 1H), 1.62-1.59 (m, 1H), 1.29 (s, 3H), 1.25 (s, 3H), 1.06 (s, 3H). 1H NMR (300 MHz, DMSO-d6) δ 9.30 (s, 1H), 8.42 (d, J = 8.1 Hz, 1H), 8.04 (s, 1H), 7.83 (t, J = 8.1 Hz, 1H), 7.72-7.63 (m, 3H), 7.19 (d, J = 8.7 Hz, 2H), 4.63- 4.59 (m, 1H), 4.51-4.46 (m, 2H), 4.30- 4.17 (m, 2H), 3.85-3.76 (m, 1H), 3.73- 3.62 (m, 1H), 3.28-3.24 (m, 1H), 2.87 (t, J = 8.4 Hz, 1H), 2.67-2.58 (m, 2H), 2.41- 2.23 (m, 6H), 1.85-1.69 (m, 1H), 1.66- 1.50 (m, 1H), 1.30 (s, 3H), 1.26 (s, 3H), 1.06 (s, 3H). | LCMS (ES, m/z): 556 [M + H]+, Rt 1.227 min. LCMS (ES, m/z): 556 [M + H]+, Rt 0.650 min. |
| 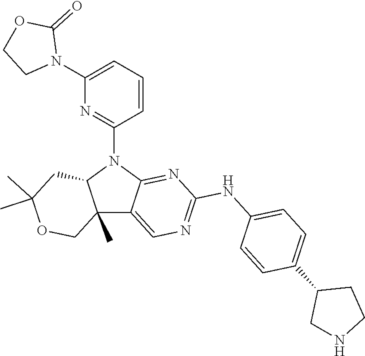 [SMILES: CC1(C)C[C@@H]2N(c3cccc(N4CCOC4=O)n3)c3nc(Nc4ccc([C@@H]5CCNC5)cc4)ncc3[C@]2(C)CO1] 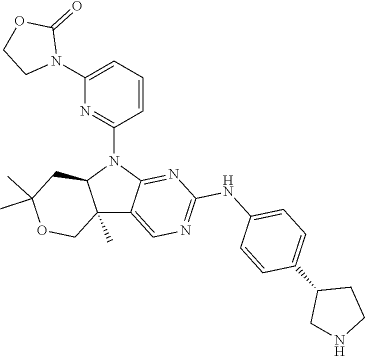 [SMILES: CC1(C)C[C@H]2N(c3cccc(N4CCOC4=O)n3)c3nc(Nc4ccc([C@@H]5CCNC5)cc4)ncc3[C@@]2(C)CO1] | 606, 607 | 1H NMR (300 MHz, DMSO-d6) δ 9.32 (s, 1H), 8.42 (d, J = 7.8 Hz, 1H), 8.05 (s, 1H), 7.86-7.81 (m, 1H), 7.72-7.64 (m, 3H), 7.21 (t, J = 7.8 Hz, 2H), 4.64-4.59 (m, 1H), 4.57-4.41 (m, 2H), 4.31-4.18 (m, 2H), 3.84-3.78 (m, 1H), 3.77-3.64 (m, 1H), 3.28-2.97 (m, 4H), 2.77-2.62 (m, 1H), 2.35-2.25 (m, 1H), 2.23-2.07 (m, 1H), 1.81-1.69 (m, 1H), 1.68-1.54 (m, 1H), 1.30 (s, 3H), 1.28 (s, 3H), 1.06 (s, 3H). 1H NMR (300 MHz, DMSO-d6) δ 9.31 (s, 1H), 8.42 (d, J = 8.1 Hz, 1H), 8.04 (s, 1H), 7.83 (t, J = 8.1 Hz, 1H), 7.72-7.63 (m, 3H), 7.19 (d, J = 8.4 Hz, 2H), 4.64- 4.46 (m, 3H), 4.31-4.18 (m, 2H), 3.86- 3.77 (m, 1H), 3.71-3.64 (m, 1H), 3.22- 3.16 (m, 2H), 3.11-2.93 (m, 2H), 2.70- 2.56 (m, 1H), 2.33-2.26 (m, 1H), 2.19- 2.06 (m, 1H), 1.79-1.56 (m, 2H), 1.30 (s, 3H), 1.26 (s, 3H), 1.07 (s, 3H). | LCMS (ES, m/z): 542 [M + H]+, Rt 0.642 min. LCMS (ES, m/z): 542 [M + H]+, Rt 0.650 min. |
| 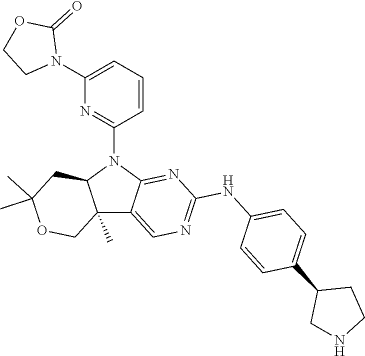 [SMILES: CC1(C)C[C@H]2N(c3cccc(N4CCOC4=O)n3)c3nc(Nc4ccc([C@H]5CCNC5)cc4)ncc3[C@@]2(C)CO1] 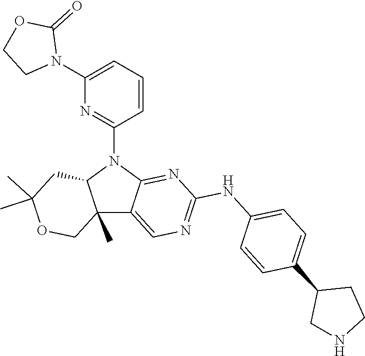 [SMILES: CC1(C)C[C@@H]2N(c3cccc(N4CCOC4=O)n3)c3nc(Nc4ccc([C@H]5CCNC5)cc4)ncc3[C@]2(C)CO1] | 608, 609 | 1H NMR (300 MHz, DMSO-d6) δ 9.35 (s, 1H), 8.41 (d, J = 8.4 Hz, 1H), 8.04 (s, 1H), 7.83 (t, J = 8.1 Hz, 1H), 7.71-7.66 (m, 3H), 7.22 (d, J = 8.4 Hz, 2H), 4.63- 4.58 (m, 1H), 4.50-4.45 (m, 2H), 4.29- 4.16 (m, 2H), 3.81-3.77 (m, 1H), 3.69- 3.65 (m, 1H), 3.42-3.36 (m, 1H), 3.26- 3.18 (m, 2H), 3.13-3.04 (m, 1H), 2.91- 2.84 (m, 1H), 2.31-2.20 (m, 2H), 1.87- 1.80 (m, 1H), 1.63-1.56 (m, 1H), 1.29 (s, 3H), 1.25 (s, 3H), 1.05 (s, 3H). 1H NMR (300 MHz, DMSO-d6) 8 9.32 (s, 1H), 8.41 (d, J = 8.1 Hz, 1H), 8.03 (s, 1H), 7.85-7.80 (m, 1H), 7.71-7.62 (m, 3H), 7.22-7.17 (m, 2H), 4.62-4.58 (m, 1H), 4.50-4.45 (m, 2H), 4.29-4.16 (m, 2H), 3.81-3.77 (m, 1H), 3.69-3.65 (m, 1H), 3.51-3.48 (m, 1H), 3.31-3.29 (m, 1H), 3.21-3.15 (m, 1H), 3.10-2.92 (m, 2H), 2.67-2.61 (m, 1H), 2.33-2.25 (m, 1H), 2.18-1.88 (m, 1H), 1.73-1.55 (m, 2H), 1.29 (s, 3H), 1.25 (s, 3H), 1.05 (s, 3H). | LCMS (ES, m/z): 542 [M + H]+, Rt 0.644 min. LCMS (ES, m/z): 542 [M + H]+, Rt 0.651min. |
| 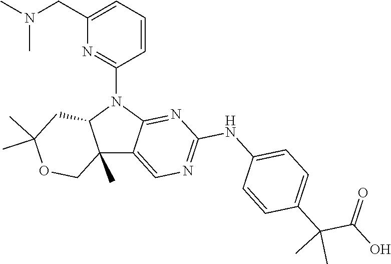 [SMILES: CN(C)Cc1cccc(N2c3nc(Nc4ccc(C(C)(C)C(=O)O)cc4)ncc3[C@]3(C)COC(C)(C)C[C@H]23)n1] 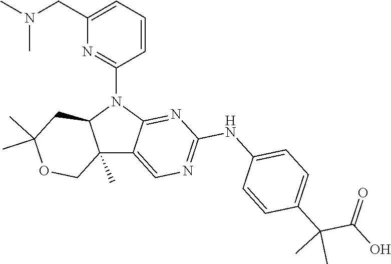 [SMILES: CN(C)Cc1cccc(N2c3nc(Nc4ccc(C(C)(C)C(=O)O)cc4)ncc3[C@@]3(C)COC(C)(C)C[C@@H]23)n1] | 610, 611 | 1H NMR (300 MHz, DMSO-d6) δ 12.21 (br, 1H), 9.31 (s, 1H), 8.52 (d, J = 8.4 Hz, 1H), 8.02 (s, 1H), 7.75 (t, J = 8.1 Hz, 1H), 7.68 (d, J = 8.7 Hz, 2H), 7.27 (d, J = 8.7 Hz, 2H), 7.08 (d, J = 7.2 Hz, 1H), 4.67- 4.63 (m, 1H), 3.79-3.63 (m, 2H), 3.56- 3.45 (m, 2H), 2.23-2.16 (m, 7H), 1.69- 1.62 (m, 1H), 1.47 (s, 6H), 1.28 (s, 3H), 1.21 (s, 3H), 1.00 (s, 3H). 1H NMR (300 MHz, DMSO-d6) δ 12.22 (br, 1H), 9.31 (s, 1H), 8.52 (d, J = 8.4 Hz, 1H), 8.03 (s, 1H), 7.73 (t, J = 8.1 Hz, 1H), 7.68 (d, J = 8.7 Hz, 2H), 7.27 (d, J = 9.0 Hz, 2H), 7.08 (d, J = 7.2 Hz, 1H), 4.67- 4.63 (m, 1H), 3.79-3.63 (m, 2H), 3.56- 3.45 (m, 2H), 2.24-2.16 (m, 7H), 1.70- 1.62 (m, 1H), 1.47 (s, 6H), 1.28 (s, 3H), 1.21 (s, 3H), 1.00 (s, 3H) | LCMS (ES, m/z): 531.35 [M + H]+, Rt 0.617 min. LCMS (ES, m/z): 531.30 [M + H]+, Rt 0.617 min. |
| 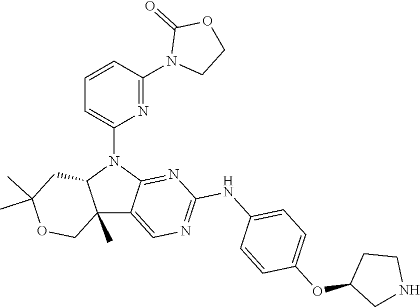 [SMILES: CC1(C)C[C@@H]2N(c3cccc(N4CCOC4=O)n3)c3nc(Nc4ccc(O[C@H]5CCNC5)cc4)ncc3[C@]2(C)CO1] 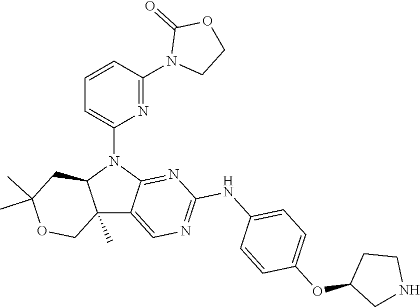 [SMILES: CC1(C)C[C@H]2N(c3cccc(N4CCOC4=O)n3)c3nc(Nc4ccc(O[C@H]5CCNC5)cc4)ncc3[C@@]2(C)CO1] | 612, 613 | 1H NMR (400 MHz, DMSO-d6) δ 9.18 (s, 1H), 8.40 (d, J = 5.6 Hz, 1H), 8.00 (s, 1H), 7.82-7.78 (m, 1H), 7.69-7.67 (m, 1H), 7.60 (d, J = 8.8 Hz, 2H), 6.87 (d, J = 9.2 Hz, 2H), 4.85-4.82 (m, 1H), 4.60- 4.44 (m, 3H), 4.28-4.16 (m, 2H), 3.79- 3.76 (m, 1H), 3.67-3.64 (m, 1H), 3.12- 3.07 (m, 1H), 2.97-2.89 (m, 3H), 2.30- 2.25 (m, 1H), 2.06-1.97 (m, 1H), 1.84- 1.78 (m, 1H), 1.61-1.57 (m, 1H), 1.27 (s, 3H), 1.24 (s, 3H), 1.05 (s, 3H). 1H NMR (400 MHz, DMSO-d6) δ 9.18 (s, 1H), 8.39 (d, J = 7.6 Hz, 1H), 8.00 (s, 1H), 7.82-7.78 (m, 1H), 7.69-7.58 (m, 3H), 6.87 (d, J = 9.2 Hz, 2H), 4.84-4.82 (m, 1H), 4.60-4.57 (m, 1H), 4.49-4.44 (m, 2H), 4.28-4.16 (m, 2H), 3.79-3.76 (m, 1H), 3.67-3.64 (m, 1H), 3.13-3.09 (m, 1H), 2.98-2.91 (m, 3H), 2.29-2.25 (m, 1H), 2.07-1.98 (m, 1H), 1.85-1.79 (m, 1H), 1.61-1.57 (m, 1H), 1.27 (s, 3H), 1.24 (s, 3H), 1.05 (s, 3H). | LCMS (ES, m/z): 558 M + H]+. Rt 0.654 min. LCMS (ES, m/z): 558 [M + H]+. Rt 0.663 min. |
| 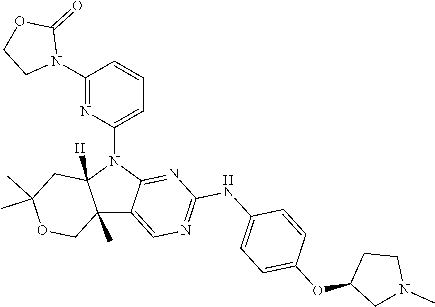 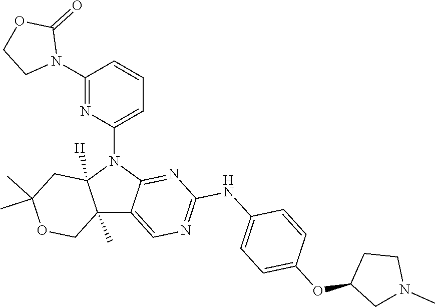 | 614, 615 | 1H NMR (400 MHz, DMSO-d6) δ 9.17 (s, 1H), 8.39 (d, J = 8.4 Hz, 1H), 8.00 (s, 1H), 7.81 (t, J = 8.0 Hz, 1H), 7.69-7.57 (m, 3H), 6.84 (d, J = 9.2 Hz, 2H), 4.84- 4.79 (m, 1H), 4.60-4.57 (m, 1H), 4.50- 4.45 (m, 2H), 4.29-4.14 (m, 2H), 3.80- 3.65 (m, 2H), 2.80-2.76 (m, 1H), 2.67- 2.56 (m, 2H), 2.39-2.56 (m, 6H), 1.80- 1.72 (m, 1H), 1.61-1.55 (m, 1H), 1.28 (s, 3H), 1.24 (s, 3H), 1.05 (s, 3H). 1H NMR (400 MHz, DMSO-d6) δ 9.17 (s, 1H), 8.39 (d, J = 8.4 Hz, 1H), 8.00 (s, 1H), 7.81 (t, J = 8.4 Hz, 1H), 7.69-7.57 (m, 3H), 6.84 (d, J = 9.2 Hz, 2H), 4.83- 4.79 (m, 1H), 4.61-4.45 (m, 3H), 4.31- 4.14 (m, 2H), 3.80-3.65 (m, 2H), 2.80- 2.76 (m, 1H), 2.67-2.56 (m, 2H), 2.39- 2.32 (m, 1H), 2.29-2.26 (m, 5H), 1.80- 1.73 (m, 1H), 1.61-1.55 (m, 1H), 1.28 (s, 3H), 1.24 (s, 3H), 1.05 (s, 3H). | LCMS (ES, m/z): 572 [M + H]+, Rt 0.664 min. LCMS (ES, m/z): 572 [M + H]+, Rt 0.663 min. |
| 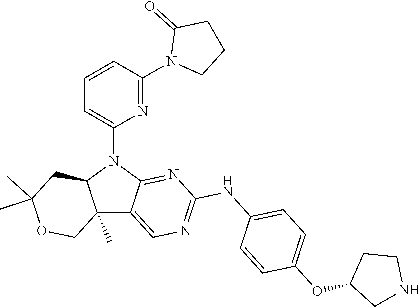 [SMILES: CC1(C)C[C@H]2N(c3cccc(N4CCCC4=O)n3)c3nc(Nc4ccc(O[C@@H]5CCNC5)cc4)ncc3[C@@]2(C)CO1] 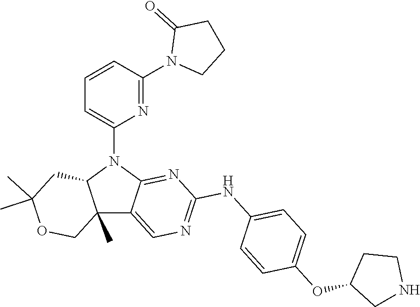 [SMILES: CC1(C)C[C@@H]2N(c3cccc(N4CCCC4=O)n3)c3nc(Nc4ccc(O[C@@H]5CCNC5)cc4)ncc3[C@]2(C)CO1] | 616, 617 | 1H NMR (300 MHz, DMSO-d6) δ 9.16 (s, 1H), 8.38 (d, J = 8.1 Hz, 1H), 7.99 (s, 1H), 7.92 (d, J = 8.1 Hz, 1H), 7.85-7.69 (m, 1H), 7.69-7.54 (m, 2H), 6.95-6.75 (m, 2H), 4.95-4.75 (m, 1H), 4.75-4.49 (m, 1H), 4.21- 3.90 (m, 2H), 3.83-3.61 (m, 2H), 3.10-2.99 (m, 1H), 2.96-2.81 (m, 2H), 2.76-2.53 (m, 3H), 2.34-2.22 (m, 1H), 2.18-1.85 (m, 3H), 1.81-1.65 (m, 1H), 1.65-1.52 (m, 1H), 1.28 (s, 3H), 1.25 (s, 3H), 1.05 (s, 3H). 1H NMR (300 MHz, DMSO-d6) δ 9.16 (s, 1H), 8.38 (d, J = 8.1 Hz, 1H), 8.00 (s, 1H), 7.92 (d, J = 7.2 Hz, 1H), 7.85-7.69 (m, 1H), 7.69-7.54 (m, 2H), 6.95-6.75 (m, 2H), 4.95-4.75 (m, 1H), 4.75-4.49 (m, 1H), 4.21- 3.90 (m, 2H), 3.83-3.61 (m, 2H), 3.10-2.99 (m, 1H), 2.96-2.81 (m, 2H), 2.76-2.53 (m, 3H), 2.34-2.22 (m, 1H), 2.18-1.85 (m, 3H), 1.81-1.65 (m, 1H), 1.65-1.52 (m, 1H), 1.28 (s, 3H), 1.25 (s, 3H), 1.06 (s, 3H). | LCMS(ES, m/z): 556 [M + H]+. Rt 0.680 min. LCMS(ES, m/z): 556 [M + H]+. Rt 0.678 min. |
| 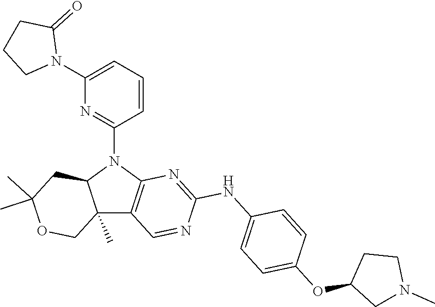 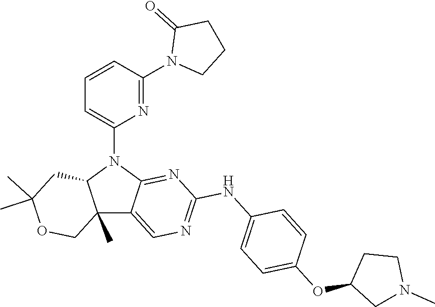 | 618, 619 | 1H NMR (300 MHz, DMSO-d6) δ 9.17 (s, 1H), 8.38 (d, J = 8.1 Hz, 1H), 8.12-7.85 (m, 2H), 7.83 (t, J = 8.1 Hz, 1H), 7.57 (d, J = 9.0 Hz, 2H), 6.83 (d, J = 9.0 Hz, 2H), 4.95-4.75 (m, 1H), 4.75-4.49 (m, 1H), 4.15-3.90 (m, 2H), 3.83-3.61 (m, 2H), 32.89-2.65 (m, 4H), 2.45-2.33 (m, 1H), 2.31-2.22 (m, 6H), 2.18-1.85 (m, 2H), 1.81-1.65 (m, 1H), 1.65-1.52 (m, 1H), 1.28 (s, 3H), 1.25 (s, 3H), 1.05 (s, 3H). 1H NMR (300 MHz, DMSO-d6) δ 9.17 (s, 1H), 8.38 (d, J = 8.1 Hz, 1H), 8.12-7.85 (m, 2H), 7.83 (t, J = 8.1 Hz, 1H), 7.57 (d, J = 9.0 Hz, 2H), 6.83 (d, J = 9.0 Hz, 2H), 4.95-4.75 (m, 1H), 4.75-4.49 (m, 1H), 4.15-3.90 (m, 2H), 3.83-3.61 (m, 2H), 32.89-2.65 (m, 4H), 2.45-2.33 (m, 1H), 2.31-2.22 (m, 6H), 2.18-1.85 (m, 2H), 1.81-1.65 (m, 1H), 1.65-1.52 (m, 1H), 1.28 (s, 3H), 1.25 (s, 3H), 1.05 (s, 3H). | LCMS(ES, m/z): 570 [M + H]+. Rt 0.690 min. LCMS(ES, m/z): 570 [M + H]+. Rt 0.691 min. |
| 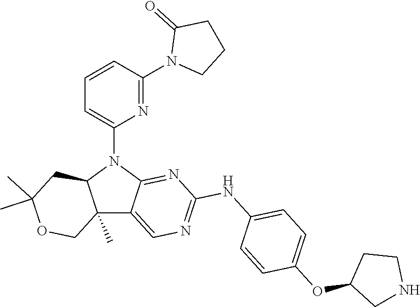 [SMILES: CC1(C)C[C@H]2N(c3cccc(N4CCCC4=O)n3)c3nc(Nc4ccc(O[C@H]5CCNC5)cc4)ncc3[C@@]2(C)CO1] 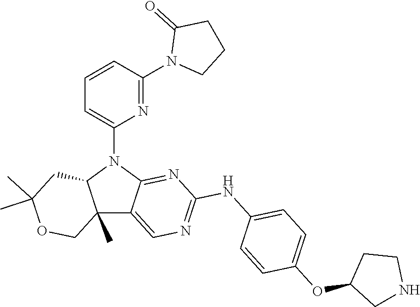 [SMILES: CC1(C)C[C@@H]2N(c3cccc(N4CCCC4=O)n3)c3nc(Nc4ccc(O[C@H]5CCNC5)cc4)ncc3[C@]2(C)CO1] | 620, 621 | 1H NMR (300 MHz, DMSO-d6) δ 9.16 (s, 1H), 8.38 (d, J = 8.1 Hz, 1H), 8.12 -7.85 (m, 2H),7.85-7.69 (m, 1H), 7.69-7.54 (m, 2H), 6.95-6.75 (m, 2H), 4.95-4.75 (m, 1H), 4.75-4.49 (m, 1H), 4.21- 3.90 (m, 2H), 3.83-3.61 (m, 2H), 3.10-2.99 (m, 1H), 2.96-2.81 (m, 2H), 2.76-2.53 (m, 3H), 2.34-2.22 (m, 1H), 2.18-1.85 (m, 3H), 1.81-1.65 (m, 1H), 1.65-1.52 (m, 1H), 1.28 (s, 3H), 1.25 (s, 3H), 1.05 (s, 3H). 1H NMR (300 MHz, DMSO-d6) δ 9.16 (s, 1H), 8.38 (d, J = 8.1 Hz, 1H), 8.12 -7.85 (m, 2H), 7.85-7.69 (m,1H), 7.69-7.54 (m, 2H), 6.95-6.75 (m, 2H), 4.95-4.75 (m, 1H), 4.75-4.49 (m, 1H), 4.21- 3.90 (m, 2H), 3.83-3.61 (m, 2H), 3.10-2.99 (m, 1H), 2.96-2.81 (m, 2H), 2.76-2.53 (m, 3H), 2.34-2.22 (m, 1H), 2.18-1.85 (m, 3H), 1.81-1.65 (m, 1H), 1.65-1.52 (m, 1H), 1.28 (s, 3H), 1.25 (s, 3H), 1.05 (s, 3H). | LCMS(ES, m/z): 556 [M + H]+. Rt 0.679 min. LCMS(ES, m/z): 556 [M + H]+. Rt 0.676 min. |
| 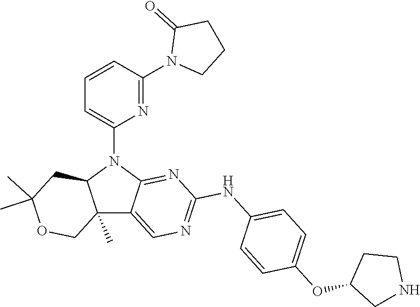 [SMILES: CC1(C)C[C@H]2N(c3cccc(N4CCCC4=O)n3)c3nc(Nc4ccc(O[C@@H]5CCNC5)cc4)ncc3[C@@]2(C)CO1] 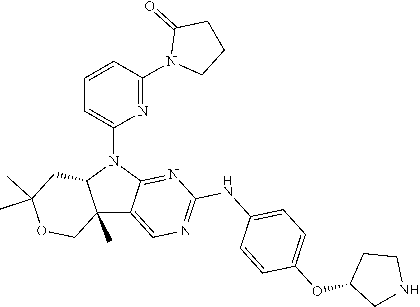 [SMILES: CC1(C)C[C@@H]2N(c3cccc(N4CCCC4=O)n3)c3nc(Nc4ccc(O[C@@H]5CCNC5)cc4)ncc3[C@]2(C)CO1] | 622, 623 | 1H NMR (300 MHz, DMSO-d6) δ 9.24 (s, 1H), 8.41 (d, J = 8.1 Hz, 1H), 8.03 (s, 1H), 7.83 (t, J = 8.1 Hz, 1H), 7.71 (d, J = 8.1 Hz, 1H), 7.61 (d, J = 9.0 Hz, 2H), 6.89 (d, J = 9.0 Hz, 2H), 4.99-4.89 (m, 1H), 4.67-4.56 (m, 1H), 4.56-4.41 (m, 2H), 4.37-4.13 (m, 2H), 3.86-3.66 (m, 2H), 3.30-3.18 (m, 1H), 3.18-2.96 (m, 3H), 2.36-2.24 (m, 1H), 2.20-2.02 (m, 1H), 2.01-1.86 (m, 1H), 1.68-1.54 (m, 1H), 1.30 (s, 3H), 1.27 (s, 3H), 1.08 (s, 3H). 1H NMR (300 MHz, DMSO-d6) δ 9.21 (s, 1H), 8.41 (d, J = 8.1 Hz, 1H), 8.03 (s, 1H), 7.83 (t, J = 8.1 Hz, 1H), 7.71 (d, J = 8.1 Hz, 1H), 7.58 (d, J = 9.0 Hz, 2H), 6.86 (d, J = 8.7 Hz, 2H), 4.87-4.81 (m, 1H), 4.67-4.56 (m, 1H), 4.56-4.41 (m, 2H), 4.37-4.13 (m, 2H), 3.86-3.65 (m, 2H), 3.15-3.03 (m, 1H), 3.03-2.76 (m, 3H), 2.36-2.24 (m, 1H), 2.12-1.94 (m, 1H), 1.89-1.74 (m, 1H), 1.68-1.54 (m, 1H), 1.30 (s, 3H), 1.27 (s, 3H), 1.08 (s, 3H). | LCMS (ES, m/z): 558 [M + H]+; RT: 0.652 min. LCMS (ES, m/z): 558 [M + H]+; RT: 0.658 min. |
| 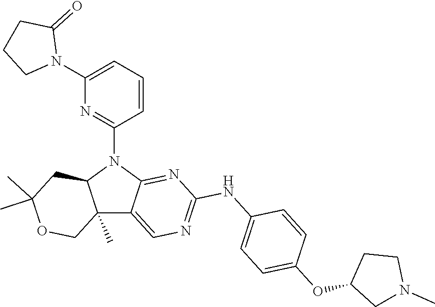 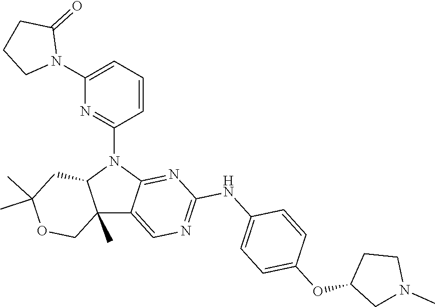 | 624, 625 | 1H NMR (300 MHz, DMSO-d6) δ 9.22 (s, 1H), 8.42 (d, J = 8.1 Hz, 1H), 8.04 (s, 1H), 7.85 (t, J = 8.1 Hz, 1H), 7.72 (d, J = 8.1 Hz, 1H), 7.58 (d, J = 9.0 Hz, 2H), 6.83 (d, J = 9.0 Hz, 2H), 4.93-4.80 (m, 1H), 4.68-4.57 (m, 1H), 4.57-4.42 (m, 2H), 4.39-4.14 (m, 2H), 3.88-3.65 (m, 2H), 2.90-2.79 (m, 1H), 2.78-2.54 (m, 2H), 2.50-2.40 (m, 1H), 2.32 (s, 3H), 2.31- 2.22 (m, 2H), 1.89-1.73 (m, 1H), 1.69- 1.55 (m, 1H), 1.32 (s, 3H), 1.28 (s, 3H), 1.09 (s, 3H). 1H NMR (300 MHz, DMSO-d6) δ 9.21 (s, 1H), 8.41 (d, J = 8.1 Hz, 1H), 8.03 (s, 1H), 7.84 (t, J = 8.1 Hz, 1H), 7.71 (d, J = 8.1 Hz, 1H), 7.59 (d, J = 9.0 Hz, 2H), 6.83 (d, J = 9.0 Hz, 2H), 4.92-4.80 (m, 1H), 4.67-4.57 (m, 1H), 4.57-4.41 (m, 2H), 4.37-4.13 (m, 2H), 3.86-3.65 (m, 2H), 2.90-2.79 (m, 1H), 2.79-2.61 (m, 2H), 2.50-2.40 (m, 1H), 2.32 (s, 3H), 2.30- 2.23 (m, 2H), 1.89-1.73 (m, 1H), 1.68- 1.54 (m, 1H), 1.30 (s, 3H), 1.27 (s, 3H), 1.08 (s, 3H). | LCMS (ES, m/z): 572 [M + H]+; RT: 0.663 min. LCMS (ES, m/z): 572 [M + H]+; RT: 0.662 min. |
| 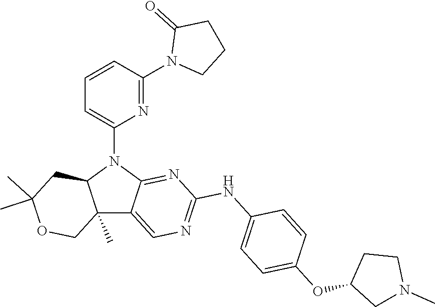 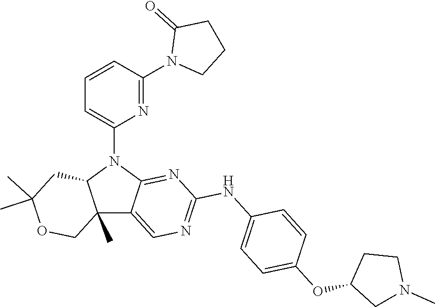 | 626, 627 | 1H NMR (400 MHz, DMSO-d6) δ 9.18 (s, 1H), 8.38 (d, J = 7.6 Hz, 1H), 8.00 (s, 1H), 7.92 (d, J = 8.0 Hz, 1H), 7.79 (t, J = 8.0 Hz, 1H), 7.59 (d, J = 9.2 Hz, 2H), 6.83 (d, J = 9.2 Hz, 2H), 4.87-4.81 (m, 1H), 4.61-4.57 (m, 1H), 4.18-3.98 (m, 2H), 3.80-3.63 (m, 2H), 2.81-2.72 (m, 1H), 2.69-2.57 (m, 4H), 2.41-2.22 (m, 6H), 2.12-2.01 (m, 2H), 1.82-1.71 (m, 1H), 1.63-1.52 (m, 1H), 1.29 (s, 3H), 1.25 (s, 3H), 1.06 (s, 3H). 1H NMR (300 MHz, DMSO-d6) δ 9.15 (s, 1H), 8.38 (d, J = 7.8 Hz, 1H), 7.99 (s, 1H), 7.92 (d, J = 7.8 Hz, 1H), 7.79 (t, J = 8.1Hz, 1H), 7.59 (d, J = 9.0 Hz, 2H), 6.83 (d, J = 9.0 Hz, 2H), 4.87-4.81 (m, 1H), 4.61-4.57 (m, 1H), 4.18-3.98 (m, 2H), 3.80-3.63 (m, 2H), 2.81-2.72 (m, 1H), 2.69-2.57 (m, 4H), 2.41-2.22 (m, 6H), 2.12-2.01 (m, 2H), 1.82-1.71 (m, 1H), 1.63-1.52 (m, 1H), 1.28 (s, 3H), 1.25 (s, 3H), 1.06 (s, 3H). | LCMS (ES, m/z): 570 [M + H]+; RT: 0.687 min. LCMS (ES, m/z): 570 [M + H]+; RT: 0.683 min. |
| 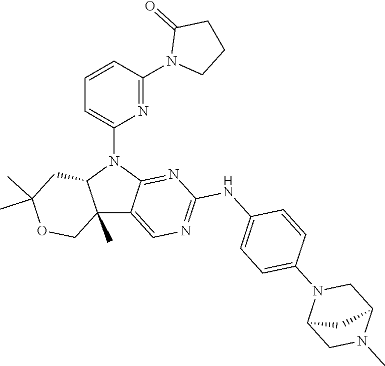 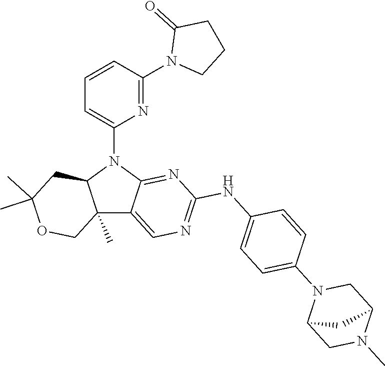 | 628, 629 | 1H NMR (300 MHz, DMSO-d6) δ 8.93 (s, 1H), 8.40 (d, J = 8.1 Hz, 1H), 7.95-7.92 (m, 2H), 7.74 (t, J = 8.1 Hz, 1H), 7.45 (d, J = 8.7 Hz, 2H), 6.56 (d, J = 8.7 Hz, 2H), 4.60-4.55 (m, 1H), 4.25 (s, 1H), 4.14- 4.06 (m, 1H), 4.01-3.93 (m, 1H), 3.81- 3.61 (m, 2H), 3.43 (s, 1H), 3.31 (s, 1H), 3.14 (d, J = 9.0 Hz, 1H), 2.81-2.73 (m, 1H), 2.60-2.55 (m, 3H), 2.29-2.27 (m, 4H), 2.10-2.03 (m, 2H), 1.88-1.85 (m, 1H), 1.78-1.75 (m, 1H), 1.61-1.53 (m, 1H), 1.27 (s, 3H), 1.24 (s, 3H), 1.05 (s, 3H). 1H NMR (300 MHz, DMSO-d6) δ 8.93 (s, 1H), 8.40 (d, J = 8.1 Hz, 1H), 7.95-7.89 (m, 2H), 7.75 (t, J = 8.1 Hz, 1H), 7.44 (d, J = 8.7 Hz, 2H), 6.56 (d, J = 9.0 Hz, 2H), 4.59-4.55 (m, 1H), 4.24 (s, 1H), 4.15- 4.06 (m, 1H), 4.01-3.92 (m, 1H), 3.79- 3.61 (m, 2H), 3.39 (s, 1H), 3.31 (s, 1H), 3.13 (d, J = 9.0 Hz, 1H), 2.79-2.71 (m, 1H), 2.60-2.55 (m, 3H), 2.31-2.25 (m, 4H), 2.10-2.00 (m, 2H), 1.87-1.83 (m, 1H), 1.77-1.74 (m, 1H), 1.61-1.53 (m, 1H), 1.27 (s, 3H), 1.24 (s, 3H), 1.05 (s, 3H). | LCMS (ES, m/z): 581 [M + H]+, Rt 0.681 min. LCMS (ES, m/z): 581 [M + H]+, Rt 0.680 min. |
| 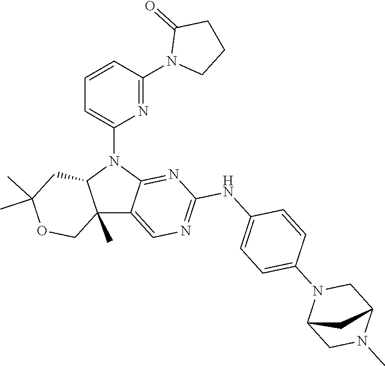 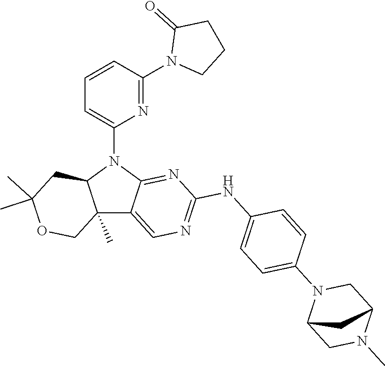 | 630, 631 | 1H NMR (300 MHz, DMSO-d6) δ 8.93 (s, 1H), 8.38 (d, J = 8.1 Hz, 1H), 7.94-7.89 (m, 2H), 7.75 (t, J = 8.1 Hz, 1H), 7.42 (d, J = 9.0 Hz, 2H), 6.53 (d, J = 8.7 Hz, 2H), 4.59-4.55 (m, 1H), 4.23 (s, 1H), 4.12- 4.08 (m, 1H), 3.98-3.94 (m, 1H), 3.78- 3.74 (m, 1H), 3.67-3.63 (m, 1H), 3.35- 3.29 (m, 3H), 3.13-3.10 (m, 1H), 2.77- 2.74 (m, 1H), 2.57-2.50 (m, 2H), 2.30- 2.21 (m, 4H), 2.07-2.02 (m, 2H), 1.86- 1.83 (m, 1H), 1.76-1.73 (m, 1H), 1.61- 1.56 (m, 1H), 1.27 (s, 3H), 1.24 (s, 3H), 1.05 (s, 3H). 1H NMR (300 MHz, DMSO-d6) δ 8.93 (s, 1H), 8.38 (d, J = 8.1 Hz, 1H), 7.94-7.89 (m, 2H), 7.75 (t, J = 8.1 Hz, 1H), 7.42 (d, J = 9.0 Hz, 2H), 6.53 (d, J = 8.7 Hz, 2H), 4.59-4.55 (m, 1H), 4.23 (s, 1H), 4.12- 4.08 (m, 1H), 3.98-3.94 (m, 1H), 3.78- 3.74 (m, 1H), 3.67-3.63 (m, 1H), 3.35- 3.29 (m, 3H), 3.13-3.10 (m, 1H), 2.77- 2.74 (m, 1H), 2.57-2.50 (m, 2H), 2.30- 2.21 (m, 4H), 2.07-2.02 (m, 2H), 1.86- 1.83 (m, 1H), 1.76-1.73 (m, 1H), 1.61- 1.56 (m, 1H), 1.27 (s, 3H), 1.24 (s, 3H), 1.05 (s, 3H). | LCMS (ES, m/z): 581 [M + H]+, Rt 0.640 min. LCMS (ES, m/z): 581 [M + H]+, Rt 0.648 min. |
| 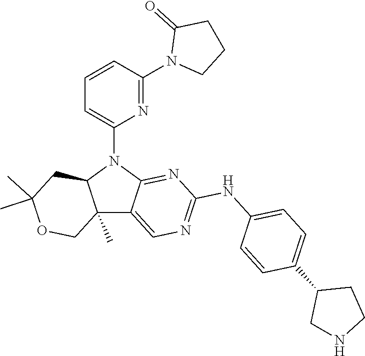 [SMILES: CC1(C)C[C@H]2N(c3cccc(N4CCCC4=O)n3)c3nc(Nc4ccc([C@@H]5CCNC5)cc4)ncc3[C@@]2(C)CO1] 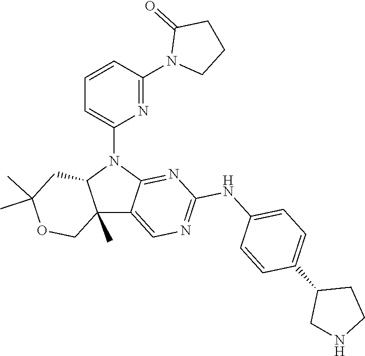 [SMILES: CC1(C)C[C@@H]2N(c3cccc(N4CCCC4=O)n3)c3nc(Nc4ccc([C@@H]5CCNC5)cc4)ncc3[C@]2(C)CO1] | 632, 633 | 1H NMR (300 MHz, DMSO-d6) δ 9.29- 9.17 (m, 1H), 8.33 (d, J = 8.1 Hz, 1H), 7.95 (s, 1H), 7.86 (d, J = 8.1 Hz, 1H), 7.72 (t, J = 8.1 Hz, 1H), 7.56 (d, J = 8.4 Hz, 2H), 7.11 (t, J = 8.7 Hz, 2H), 4.56- 4.51 (m, 1H), 4.09-3.84 (m, 2H), 3.78- 3.56 (m, 2H), 3.15-3.09 (m, 1H), 3.05- 2.77 (m, 3H), 2.59-2.49 (m, 3H), 2.26- 2.16 (m, 1H), 2.11-1.91 (m, 3H), 1.69- 1.45 (m, 3H), 1.22 (s, 3H), 1.18 (s, 3H), 0.98 (s, 3H). 1H NMR (300 MHz, DMSO-d6) δ 9.37- 9.28 (m, 1H), 8.41 (d, J = 8.1 Hz, 1H), 8.04 (s, 1H), 7.95 (d, J = 7.8 Hz, 1H), 7.80 (t, J = 8.2 Hz, 1H), 7.65 (d, J = 8.4 Hz, 2H), 7.21 (t, J = 8.6 Hz, 2H), 4.64- 4.60 (m, 1H), 4.20-4.05 (m, 1H), 4.03- 3.91 (m, 1H), 3.85-3.63 (m, 2H), 3.25- 2.95 (m, 4H), 2.69-2.52 (m, 3H), 2.36- 2.23 (m, 1H), 2.05-1.99 (m, 3H), 1.77- 1.53 (m, 2H), 1.30 (s, 3H), 1.26 (s, 3H), 1.07 (s, 3H). | LCMS (ES, m/z): 540 [M + H]+, Rt 0.653 min. LCMS (ES, m/z): 540 [M + H]+, Rt 0.660 min. |
| 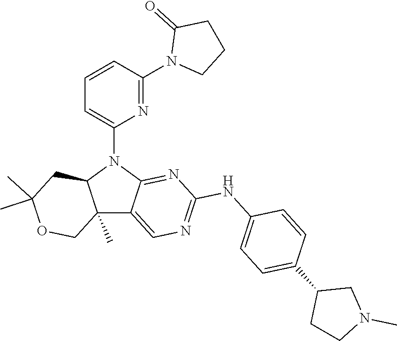 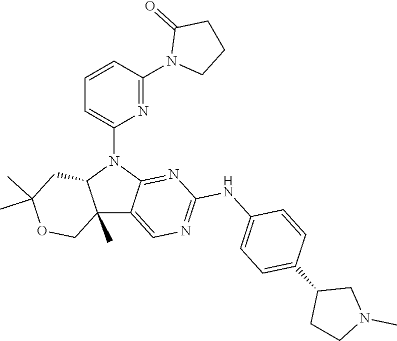 | 634, 635 | 1H NMR (300 MHz, DMSO-d6) δ 9.28 (s, 1H), 8.40 (d, J = 8.1 Hz, 1H), 8.02 (s, 1H), 7.93 (d, J = 8.1 Hz, 1H), 7.79 (t, J = 8.1 Hz, 1H), 7.63 (d, J = 8.4 Hz, 2H), 7.23-7.14 (m, 2H), 4.65-4.55 (m, 1H), 4.18-4.04 (m, 1H), 4.04-3.90 (m, 1H), 3.83-3.74 (m, 1H), 3.74-3.62 (m, 1H), 3.26-3.18 (m, 1H), 2.98-2.75 (m, 1H), 2.72 -2.53 (m, 4H), 2.43-2.14 (m, 6H), 2.14-1.98 (m, 2H), 1.84-1.66 (m, 1H), 1.66-1.52 (m, 1H), 1.29 (s, 3H), 1.25 (s, 3H), 1.05 (s, 3H). 1H NMR (300 MHz, DMSO-d6) δ 9.28 (s, 1H), 8.40 (d, J = 8.1 Hz, 1H), 8.02 (s, 1H), 7.93 (d, J = 8.1 Hz, 1H), 7.79 (t, J = 8.1 Hz, 1H), 7.63 (d, J = 8.4 Hz, 2H), 7.23-7.14 (m, 2H), 4.65-4.55 (m, 1H), 4.18-4.04 (m, 1H), 4.04-3.90 (m, 1H), 3.83-3.74 (m, 1H), 3.74-3.62 (m, 1H), 3.26-3.18 (m, 1H), 2.98-2.75 (m, 1H), 2.72 -2.53 (m, 4H), 2.43-2.14 (m, 6H), 2.14-1.98 (m, 2H), 1.84-1.66 (m, 1H), 1.66-1.52 (m, 1H), 1.29 (s, 3H), 1.25 (s, 3H), 1.05 (s, 3H). | LCMS(ES, m/z): 554 [M + H]+. Rt 0.660 min. LCMS(ES, m/z): 554 [M + H]+. Rt 0.652 min. |
| 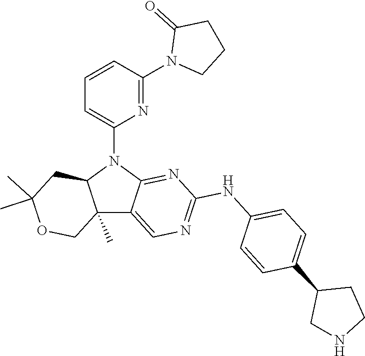 [SMILES: CC1(C)C[C@H]2N(c3cccc(N4CCCC4=O)n3)c3nc(Nc4ccc([C@H]5CCNC5)cc4)ncc3[C@@]2(C)CO1] 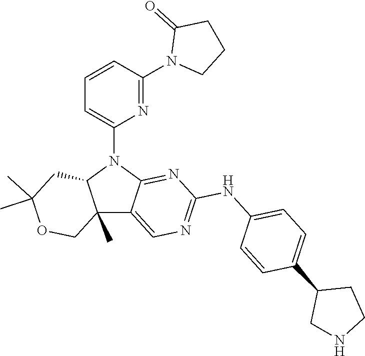 [SMILES: CC1(C)C[C@@H]2N(c3cccc(N4CCCC4=O)n3)c3nc(Nc4ccc([C@H]5CCNC5)cc4)ncc3[C@]2(C)CO1] | 636, 637 | 1H NMR (400 MHz, DMSO-d6) δ 9.35- 9.25 (m, 1H), 8.40 (d, J = 8.0 Hz, 1H), 8.05-8.00 (m, 1H), 7.93 (d, J = 8.0 Hz, 1H), 7.84-7.75 (m, 1H), 7.71-7.60 (m, 2H), 7.35-7.14 (m, 2H), 4.65-4.55 (m, 1H), 4.25-4.08 (m, 1H), 4.08-3.92 (m, 1H), 3.82-3.75 (m, 1H), 3.70-3.60 (m, 1H), 3.51-3.42 (m, 1H), 3.30-3.20 (m, 1H), 3.12-3.05 (m, 1H), 2.98-2.85 (m, 1H), 2.67-2.54 (m, 3H), 2.33-2.24 (m, 1H), 2.18-2.00 (m, 3H), 1.75-1.54 (m, 2H), 1.29 (s, 3H), 1.25 (s, 3H), 1.05 (s, 3H). 1H NMR (400 MHz, DMSO-d6) δ 9.35- 9.25 (m, 1H), 8.40 (d, J = 8.0 Hz, 1H), 8.05-8.00 (m, 1H), 7.93 (d, J = 8.0 Hz, 1H), 7.84-7.75 (m, 1H), 7.71-7.60 (m, 2H), 7.35-7.14 (m, 2H), 4.75-4.45 (m, 1H), 4.25-4.08 (m, 1H), 4.08-3.92 (m, 1H), 3.85-3.75 (m, 1H), 3.75-3.63 (m, 1H), 3.51-3.42 (m, 1H), 3.23-3.14 (m, 1H), 3.13-3.06 (m, 1H), 3.06-2.85 (m, 1H), 2.67-2.54 (m, 3H), 2.33-2.24 (m, 1H), 2.18-2.00 (m, 3H), 1.75-1.54 (m, 2H), 1.29 (s, 3H), 1.25 (s, 3H), 1.05 (s, 3H). | LCMS(ES, m/z): 540 [M + H]+. Rt 0.654 min. LCMS(ES, m/z): 540 [M + H]+. Rt 0.655 min. |
| 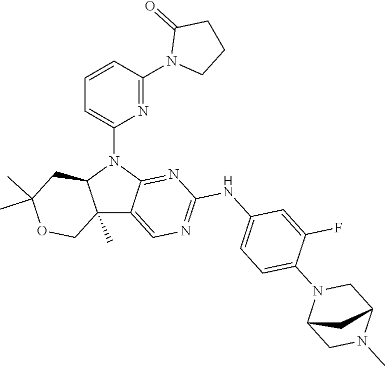 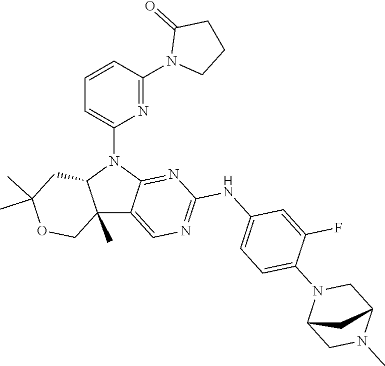 | 638, 639 | 1H NMR (300 MHz, DMSO-d6) δ 9.22 (s, 1H), 8.38 (d, J = 7.8 Hz, 1H), 8.01 (s, 1H), 7.93 (d, J = 7.8 Hz, 1H), 7.79-7.61 (m, 2H), 7.25-7.22 (m, 1H), 6.71 (t, J = 9.3 Hz, 1H), 4.62-4.57 (m, 1H), 4.23- 3.91 (m, 3H), 3.80-3.76 (m, 1H), 3.68- 3.64 (m, 1H), 3.47-3.38 (m, 1H), 3.23- 3.19 (m, 1H), 2.77-2.69 (m, 1H), 2.66- 2.56 (m, 3H), 2.33-2.20 (m, 5H), 2.13- 1.98 (m, 2H), 1.86-1.72 (m, 2H), 1.63- 1.55 (m, 1H), 1.28 (s, 3H), 1.25 (s, 3H), 1.05 (s, 3H). 1H NMR (300 MHz, DMSO-d6) δ 9.22 (s, 1H), 8.37 (d, J = 8.1 Hz, 1H), 8.01 (s, 1H), 7.93 (d, J = 8.1 Hz, 1H), 7.79-7.61 (m, 2H), 7.25-7.22 (m, 1H), 6.71 (t, J = 9.6 Hz, 1H), 4.62-4.57 (m, 1H), 4.23- 3.91 (m, 3H), 3.80-3.76 (m, 1H), 3.68- 3.64 (m, 1H), 3.47-3.38 (m, 1H), 3.23- 3.19 (m, 1H), 2.77-2.69 (m, 1H), 2.66- 2.56 (m, 4H), 2.33-2.20 (m, 4H), 2.13- 1.98 (m, 2H), 1.86-1.72 (m, 2H), 1.63- 1.55 (m, 1H), 1.28 (s, 3H), 1.24 (s, 3H), 1.05 (s, 3H). | LCMS (ES, m/z): 599 [M + H]+, Rt 0.699 min. LCMS (ES, m/z): 599 [M + H]+, Rt 0.709 min. |
| 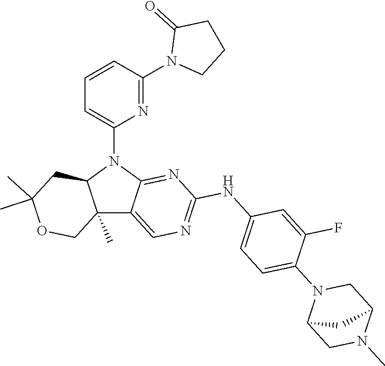 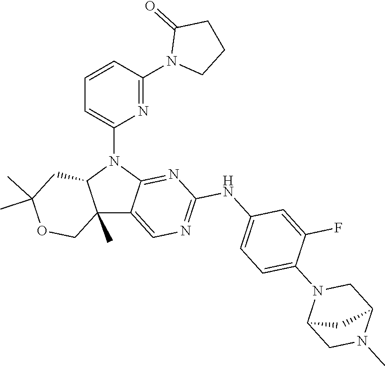 | 640, 641 | 1H NMR (300 MHz, DMSO-d6) δ 9.22 (s, 1H), 8.38 (d, J = 8.1 Hz, 1H), 8.01 (s, 1H), 7.93 (d, J = 7.8 Hz, 1H), 7.79-7.61 (m, 2H), 7.25-7.22 (m, 1H), 6.70 (t, J = 9.6 Hz, 1H), 4.62-4.57 (m, 1H), 4.20 (s, 1H), 4.15-4.07 (m, 1H), 4.01-3.93 (m, 1H), 3.80-3.76 (m, 1H), 3.68-3.64 (m, 1H), 3.51-3.42 (m, 2H), 3.23-3.19 (m, 1H), 2.78-2.56 (m, 4H), 2.29-2.25 (m, 4H), 2.08-2.03 (m, 2H), 1.86-1.72 (m, 2H), 1.63-1.55 (m, 1H), 1.28 (s, 3H), 1.25 (s, 3H), 1.05 (s, 3H). 1H NMR (300 MHz, DMSO-d6) δ 9.22 (s, 1H), 8.38 (d, J = 8.1 Hz, 1H), 8.01-7.92 (m, 2H), 7.79-7.61 (m, 2H), 7.25-7.22 (m, 1H), 6.70 (t, J = 9.2 Hz, 1H), 4.62- 4.57(m, 1H), 4.20 (s, 1H), 4.15-4.07 (m, 1H), 4.01-3.93 (m, 1H), 3.80-3.76 (m, 1H), 3.68-3.64 (m, 1H), 3.45-3.41 (m, 2H), 3.23-3.19 (m, 1H), 2.78-2.56 (m, 4H), 2.29 (s, 4H), 2.11-1.99 (m, 2H), 1.86-1.72 (m, 2H), 1.63-1.55 (m, 1H), 1.28 (s, 3H), 1.25 (s, 3H), 1.05 (s, 3H). | LCMS (ES, m/z): 599 [M + H]+, Rt 0.670 min. LCMS (ES, m/z): 599[M + H]+, Rt 1.895 min. |
| 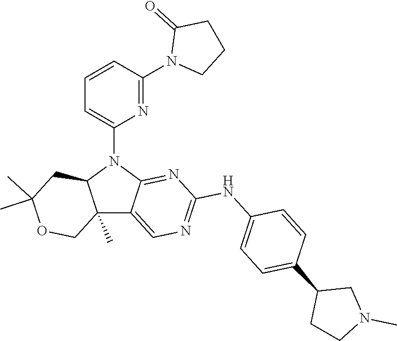 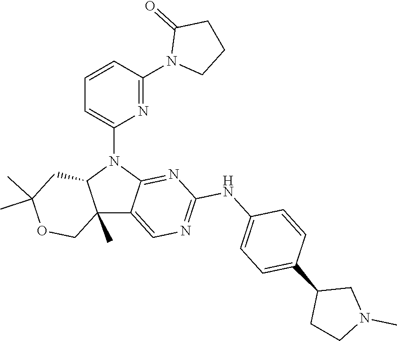 | 642, 643 | 1H NMR (300 MHz, DMSO-d6) δ 9.28 (s, 1H), 8.40 (d, J = 8.1 Hz, 1H), 8.02-7.92 (m, 2H), 7.82-7.62 (m, 3H), 7.19 (d, J = 8.4 Hz, 2H), 4.62-.58 (m, 1H), 4.17-4.04 (m, 1H), 4.03-3.96 (m, 1H), 3.81-3.65 (m, 2H), 3.29-3.18 (m, 1H), 2.90 (t, J = 8.4 Hz, 1H), 2.69-2.56 (m, 4H), 2.40 (t, J = 8.4 Hz, 1H), 2.32-2.23 (m, 5H), 2.08- 2.03 (m, 2H), 1.82-1.73 (m, 1H), 1.63- 1.55 (m, 1H), 1.29 (s, 3H), 1.25 (s, 3H), 1.05 (s, 3H). 1H NMR (300 MHz, DMSO-d6) δ 9.29 (s, 1H), 8.40 (d, J = 8.1 Hz, 1H), 8.02 (s, 1H), 7.93 (d, J = 8.1 Hz, 1H), 7.79 (t, J = 8.1 Hz, 1H), 7.63 (d, J = 8.4 Hz, 2H), 7.18 (d, J = 8.4 Hz, 2H), 4.62-4.58 (m, 1H), 4.17-4.05 (m, 1H), 4.01-3.92 (m, 1H), 3.83-3.62 (m, 2H), 3.25 (t, J = 8.4 Hz, 1H), 2.91 (t, J = 8.4 Hz, 1H), 2.70-2.55 (m, 4H), 2.49-2.39 (m, 1H), 2.33-2.23 (m, 5H), 2.08-2.01 (m, 2H), 1.82-1.71 (m, 1H), 1.63-1.56 (m, 1H), 1.29 (s, 3H), 1.25 (s, 3H), 1.05 (s, 3H). | LCMS (ES, m/z): 554[M + H]+, Rt 0.684 min. LCMS (ES, m/z): 554[M + H]+, Rt 0.690 min. |
| 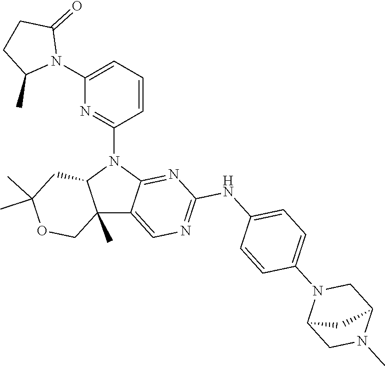 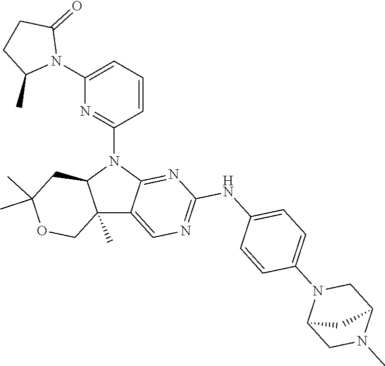 | 650, 651 | 1H NMR (400 MHz, DMSO-d6) δ 8.95 (s, 1H), 8.55-8.28 (m, 1H), 7.96 (s, 1H), 7.89 (d, J = 8.0 Hz, 1H), 7.74 (t, J = 8.0 Hz, 1H), 7.58-7.30 (m, 2H), 6.69-6.42 (m, 2H), 4.82-4.58 (m, 1H), 4.58-4.35 (m, 1H), 4.23 (s, 1H), 3.90-3.75 (m, 1H), 3.75-3.63 (m, 1H), 3.39 (s, 1H), 3.32- 3.30 (m, 1H), 3.20-3.00 (m, 1H), 2.83- 2.72 (m, 2H), 2.48-2.38 (m, 2H), 2.36- 2.22 (m, 5H), 2.00-1.75 (m, 1H), 1.75- 1.65 (m, 2H), 1.61-1.51 (m, 1H), 1.40 (d, J = 6.0 Hz, 3H), 1.27 (s, 3H), 1.25 (s, 3H), 1.07 (s, 3H). 1H NMR (400 MHz, DMSO-d6) δ 8.95 (s, 1H), 8.55-8.28 (m, 1H), 7.96 (s, 1H), 7.85-7.79 (m, 1H), 7.80-7.65 (m, 1H), 7.58-7.30 (m, 2H), 6.69-6.42 (m, 2H), 4.99-4.75 (m, 1H),4.75- 4.55 (m, 1H), 4.23 (s, 1H), 3.90-3.75 (m, 1H), 3.75- 3.63 (m, 1H), 3.39 (s, 1H), 3.32-3.30 (m, 1H), 3.18-3.12 (m, 1H), 2.83-2.72 (m, 2H), 2.48-2.38 (m, 2H), 2.36-2.22 (m, 5H), 2.00-1.75 (m, 1H), 1.75-1.65 (m, 2H), 1.61-1.51 (m, 1H), 1.35-1.24(m, 9H), 1.07 (s, 3H). | LCMS(ES, m/z): 595 [M + H]+. Rt 0.680 min. LCMS(ES, m/z): 595 [M + H]+. Rt 0.672 min. |
| 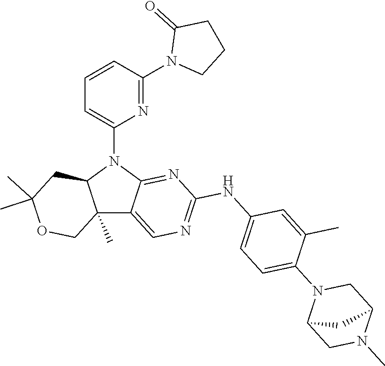 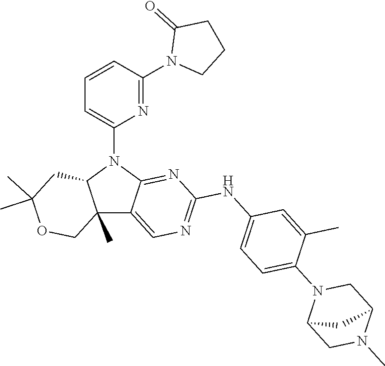 | 652, 653 | 1H NMR (400 MHz, DMSO-d6) δ 9.05 (s, 1H), 8.39 (d, J = 8.0 Hz, 1H), 7.98 (s, 1H), 7.92 (d, J = 8.0 Hz, 1H), 7.75 (t, J = 8.0 Hz, 1H), 7.45 (d, J = 2.4 Hz, 1H), 7.29 (d, J = 7.6 Hz, 1H), 6.75 (d, J = 7.2 Hz, 1H), 4.60-4.57 (m, 1H), 4.14-4.07 (m, 1H), 4.00-3.94 (m, 1H), 3.91-3.87 (m, 1H), 3.79-3.62 (m, 2H), 3.32-3.30 (m, 1H), 3.24-3.22 (m, 1H), 3.19-3.16 (m, 1H), 2.77-2.74 (m, 1H), 2.71-2.69 (m, 1H), 2.60-2.56 (m, 2H), 2.29-2.26 (m, 4H), 2.21 (s, 3H), 2.09-2.02 (m, 2H), 1.80 (d, J = 9.2 Hz, 1H), 1.72 (d, J = 9.2 Hz, 1H), 1.60-1.54 (m, 1H), 1.28 (s, 3H), 1.25 (s, 3H), 1.05 (s, 3H). 1H NMR (400 MHz, DMSO-d6) δ 9.05 (s, 1H), 8.39 (d, J = 8.0 Hz, 1H), 7.98 (s, 1H), 7.92 (d, J = 8.0 Hz, 1H), 7.75 (t, J = 8.0 Hz, 1H), 7.45 (d, J = 2.4 Hz, 1H), 7.28 (d, J = 8.4 Hz, 1H), 6.75 (d, J = 8.4 Hz, 1H), 4.60-4.57 (m, 1H), 4.14-4.07 (m, 1H), 4.00-3.94 (m, 1H), 3.91-3.87 (m, 1H), 3.81-3.61 (m, 2H), 3.32-3.30 (m, 1H), 3.24-3.17 (m, 2H), 2.77-2.67 (m, 2H), 2.60-2.56 (m, 2H), 2.30-2.26 (m, 4H), 2.21 (s, 3H), 2.09-2.02 (m, 2H), 1.81 (d, J = 8.8 Hz, 1H), 1.72 (d, J = 8.8 Hz, 1H), 1.60-1.55 (m, 1H), 1.28 (s, 3H), 1.25 (s, 3H), 1.05 (s, 3H). | LCMS (ES, m/z): 595 [M + H]+, Rt 0.669 min. LCMS (ES, m/z): 595 [M + H]+, Rt 0.677 min. |
| 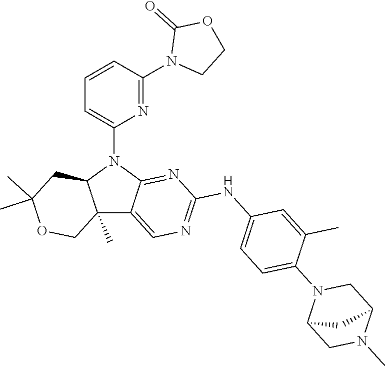 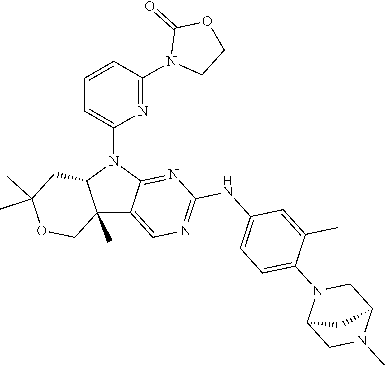 | 654, 655 | 1H NMR (400 MHz, DMSO-d6) δ 9.06 (s, 1H), 8.40 (d, J = 8.0 Hz, 1H), 7.99 (s, 1H), 7.80 (t, J = 8.0 Hz, 1H), 7.69 (d, J = 7.6 Hz, 1H), 7.45 (d, J = 2.4 Hz, 1H), 7.28 (d, J = 8.4 Hz, 1H), 6.75 (d, J = 8.4 Hz, 1H), 4.60-4.57 (m, 1H), 4.49-4.45 (m, 2H), 4.31-4.24 (m, 1H), 4.21-4.14 (m, 1H), 3.91-3.87 (m, 1H), 3.1-3.62 (m, 2H), 3.37-3.33 (m, 1H), 3.24-3.17 (m, 2H), 2.78-2.67 (m, 2H), 2.30-2.26 (m, 4H), 2.21(s, 3H), 1.81 (d, J = 9.2 Hz, 1H), 1.72 (d, J = 9.2 Hz, 1H), 1.60-1.54 (m, 1H), 1.27 (s, 3H), 1.24 (s, 3H), 1.05 (s, 3H). 1H NMR (400 MHz, DMSO-d6) δ 9.06 (s, 1H), 8.40 (d, J = 8.0 Hz, 1H), 7.99 (s, 1H), 7.80 (t, J = 8.0 Hz, 1H), 7.69 (d, J = 8.0 Hz, 1H), 7.46 (d, J = 2.0 Hz, 1H), 7.29 (d, J = 9.2 Hz, 1H), 6.76 (d, J = 8.8 Hz, 1H), 4.60-4.45 (m, 3H), 4.31-4.14 (m, 2H), 3.96-3.92 (m, 1H), 3.81-3.62 (m, 2H), 3.37-3.33 (m, 1H), 3.25-3.17 (m, 2H), 2.79-2.67 (m, 2H), 2.32-2.26 (m, 4H), 2.21(s, 3H), 1.83 (d, J = 8.8 Hz, 1H), 1.74 (d, J = 9.2 Hz, 1H), 1.60-1.54 (m, 1H), 1.27 (s, 3H), 1.24 (s, 3H), 1.05 (s, 3H). | LCMS (ES, m/z): 597 [M + H]+, Rt 0.657 min. LCMS (ES, m/z): 597 [M + H]+, Rt 0.646 min. |
| 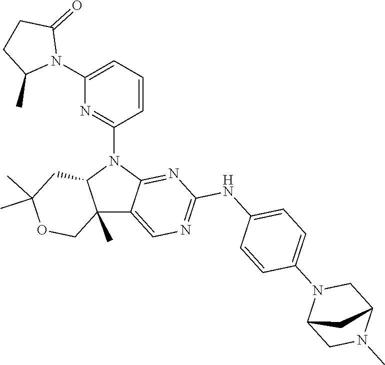 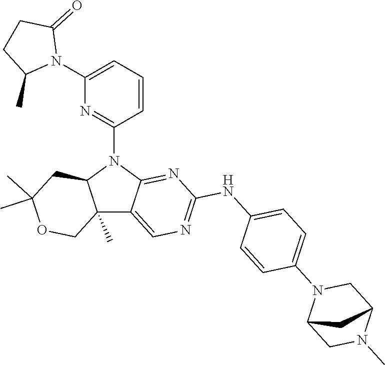 | 656, 657 | 1H NMR (400 MHz, DMSO-d6) δ 8.95 (s, 1H), 8.42-8.36 (m, 1H), 7.96 (s, 1H), 7.89 (d, J = 8.0 Hz, 1H), 7.75 (t, J = 8.0 Hz, 1H), 7.52-7.29 (m, 2H), 6.65-6.39 (m, 2H), 4.72-4.60 (m, 1H), 4.60-4.35 (m, 1H), 4.23 (s, 1H), 3.90-3.74 (m, 1H), 3.74-3.55 (m, 1H), 3.38 (s, 1H), 3.31- 3.28 (m, 1H), 3.20-3.04 (m, 1H), 2.95- 2.71 (m, 2H), 2.50-2.40 (m, 2H), 2.35- 2.21 (m, 5H), 2.00-1.81 (m, 1H), 1.81- 1.68 (m, 2H), 1.68-1.51 (m, 1H), 1.40 (d, J = 6.0 Hz, 3H), 1.27 (s, 3H), 1.25 (s, 3H), 1.07 (s, 3H). 1H NMR (400 MHz, DMSO-d6) δ 8.95 (s, 1H), 8.42-8.36 (m, 1H), 7.96 (s, 1H), 7.89 (d, J = 8.0 Hz, 1H), 7.75 (t, J = 8.0 Hz, 1H), 7.52-7.29 (m, 2H), 6.65-6.39 (m, 2H), 4.72-4.60 (m, 1H), 4.60-4.35 (m, 1H), 4.23 (s, 1H), 3.90-3.74 (m, 1H), 3.74-3.55 (m, 1H), 3.38 (s, 1H), 3.31- 3.28 (m, 1H), 3.20-3.04 (m, 1H), 2.95- 2.71 (m, 2H), 2.50-2.40 (m, 2H), 2.35- 2.21 (m, 5H), 2.00-1.81 (m, 1H), 1.81- 1.68 (m, 2H), 1.68-1.51 (m, 1H), 1.29 (d, J = 6.0 Hz, 3H), 1.28 (s, 3H), 1.24 (s, 3H), 1.07 (s, 3H). | LCMS(ES, m/z): 595 [M + H]+. Rt 0.673 min. LCMS(ES, m/z): 595 [M + H]+. Rt 0.666 min. |
| 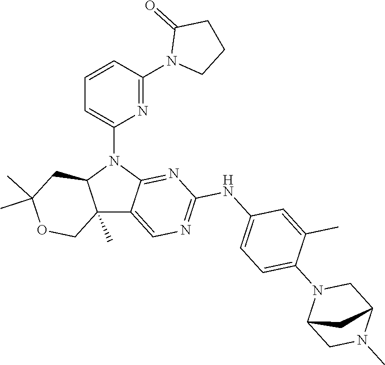 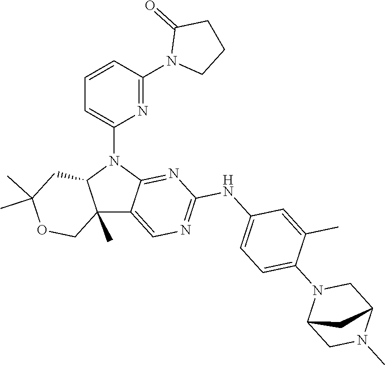 | 658, 659 | 1H NMR (300 MHz, DMSO-d6) δ 9.03 (s, 1H), 8.38 (d, J = 8.1 Hz, 1H), 7.97 (s, 1H), 7.91 (d, J = 7.8 Hz, 1H), 7.75 (t, J = 8.1 Hz, 1H), 7.45-7.44 (m, 1H), 7.28- 7.26 (m, 1H), 6.74 (d, J = 8.7 Hz, 1H), 4.59-4.56 (m, 1H), 4.17-4.08 (m, 1H), 3.95-3.93 (m, 1H), 3.92-3.89 (m, 1H), 3.79-3.75 (m, 1H), 3.67-3.63 (m, 1H), 3.24-3.18 (m, 2H), 2.74-2.68 (m, 2H), 2.60-2.54 (m, 2H), 2.50-2.49 (m, 1H), 2.29-2.25 (m, 4H), 2.21 (s, 3H), 2.07- 2.03 (m, 2H), 1.1-1.77 (m, 1H), 1.71- 1.69 (m, 1H), 1.62-1.58 (m, 1H), 1.27 (s, 3H), 1.24 (s, 3H), 1.05 (s, 3H). 1H NMR (300 MHz, DMSO-d6) δ 9.04 (s, 1H), 8.38 (d, J = 8.1 Hz, 1H), 7.97 (s, 1H), 7.91 (d, J = 7.8 Hz, 1H), 7.75 (t, J = 8.2 Hz, 1H), 7.45-7.44 (m, 1H), 7.28- 7.26 (m, 1H), 6.74 (d, J = 8.7 Hz, 1H), 4.59-4.56 (m, 1H), 4.17-4.08 (m, 1H), 3.95-3.93 (m, 1H), 3.92-3.88 (m, 1H), 3.79-3.75 (m, 1H), 3.67-3.63 (m, 1H), 3.24-3.18 (m, 2H), 2.74-2.68 (m, 2H), 2.60-2.54 (m, 2H), 2.50-2.49 (m, 1H), 2.29-2.25 (m, 4H), 2.21 (s, 3H), 2.07- 2.03 (m, 2H), 1.1-1.77 (m, 1H), 1.71- 1.69 (m, 1H), 1.62-1.58 (m, 1H), 1.27 (s, 3H), 1.24 (s, 3H), 1.05 (s, 3H). | LCMS (ES, m/z): 595 [M + H]+, Rt 0.669 min. LCMS (ES, m/z): 595 [M + H]+, Rt 0.668 min. |
| 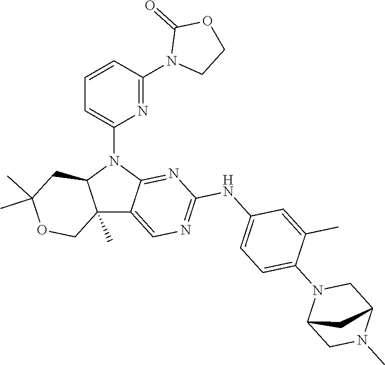 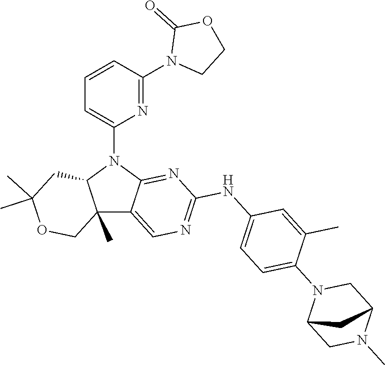 | 660, 661 | 1H NMR (300 MHz, DMSO-d6) δ 9.05 (s, 1H), 8.38 (d, J = 8.1 Hz, 1H), 7.99 (s, 1H), 7.78 (t, J = 8.1 Hz, 1H), 7.67 (d, J = 7.8 Hz, 1H), 7.45-7.44 (m, 1H), 7.29- 7.26 (m, 1H), 6.74 (d, J = 8.7 Hz, 1H), 4.60-4.55 (m, 1H), 4.50-4.44 (m, 2H), 4.28- 4.15 (m, 2H), 3.94-3.86 (m, 1H), 3.80-3.75 (m, 1H), 3.67-3.63 (m, 1H), 3.27-3.16 (m, 2H), 2.77-2.69 (m, 2H), 2.49-2.46 (m, 1H), 2.30-2.24 (m, 4H), 2.21 (s, 3H), 1.82-1.78 (m, 1H), 1.76- 1.72 (m, 1H), 1.60-1.53 (m, 1H), 1.26 (s, 3H), 1.23 (s, 3H), 1.05 (s, 3H). 1H NMR (300 MHz, DMSO-d6) δ 9.05 (s, 1H), 8.38 (d, J = 8.1 Hz, 1H), 7.99 (s, 1H), 7.78 (t, J = 8.1 Hz, 1H), 7.67 (d, J = 7.8 Hz, 1H), 7.45-7.44 (m, 1H), 7.29- 7.26 (m, 1H), 6.74 (d, J = 8.7 Hz, 1H), 4.60-4.55 (m, 1H), 4.50-4.44 (m, 2H), 4.28- 4.15 (m, 2H), 3.94-3.86 (m, 1H), 3.80-3.75 (m, 1H), 3.67-3.63 (m, 1H), 3.27-3.16 (m, 2H), 2.77-2.69 (m, 2H), 2.49-2.46 (m, 1H), 2.30-2.24 (m, 4H), 2.21 (s, 3H), 1.81-1.78 (m, 1H), 1.72- 1.69 (m, 1H), 1.61-1.53 (m, 1H), 1.27 (s, 3H), 1.24 (s, 3H), 1.05 (s, 3H). | LCMS (ES, m/z): 597 [M + H]+, Rt 0.640 min. LCMS (ES, m/z): 597 [M + H]+, Rt 0.651 min. |
| 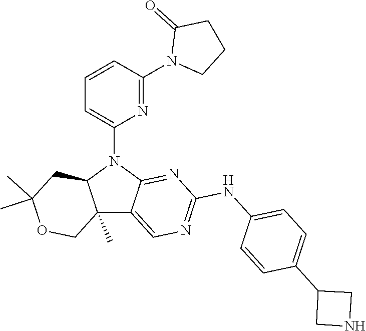 [SMILES: CC1(C)C[C@H]2N(c3cccc(N4CCCC4=O)n3)c3nc(Nc4ccc(C5CNC5)cc4)ncc3[C@@]2(C)CO1] 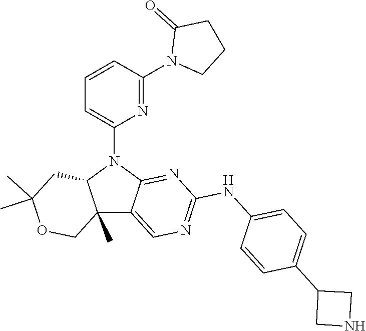 [SMILES: CC1(C)C[C@@H]2N(c3cccc(N4CCCC4=O)n3)c3nc(Nc4ccc(C5CNC5)cc4)ncc3[C@]2(C)CO1] | 662, 663 | 1H NMR (300 MHz, DMSO-d6) δ 9.34 (s, 1H), 8.40 (d, J = 8.1 Hz, 1H), 8.03 (s, 1H), 7.93 (d, J = 7.8 Hz, 1H), 7.80 (t, J = 8.1 Hz, 1H), 7.69 (d, J = 8.1 Hz, 2H), 7.26 (d, J = 8.4 Hz, 2H), 4.63-4.58 (m, 1H), 4.13-3.93 (m, 3H), 3.81-3.65 (m, 6H), 2.61-2.56 (m, 2H), 2.32-2.26 (m, 1H), 2.10-2.00 (m, 2H), 1.63-1.55 (m, 1H), 1.29 (s, 3H), 1.26 (s, 3H), 1.05 (s, 3H). 1H NMR (300 MHz, DMSO-d6) δ 9.34 (s, 1H), 8.40 (d, J = 8.1 Hz, 1H), 8.03 (s, 1H), 7.93 (d, J = 7.8 Hz, 1H), 7.80 (t, J = 8.1 Hz, 1H), 7.69 (d, J = 7.8 Hz, 2H), 7.26 (d, J = 8.4 Hz, 2H), 4.63-4.58 (m, 1H), 4.14-4.07 (m, 2H), 4.03-3.90 (m, 1H), 3.81-3.65 (m, 6H), 2.61-2.58 (m, 2H), 2.32-2.26 (m, 1H), 2.11-1.99 (m, 2H), 1.63- 1.55 (m, 1H), 1.29 (s, 3H), 1.25 (s, 3H), 1.05 (s, 3H). | LCMS (ES, m/z): 526 [M + H]+, Rt 0.635 min. LCMS (ES, m/z): 526 [M + H]+, Rt 0.631 min. |
| 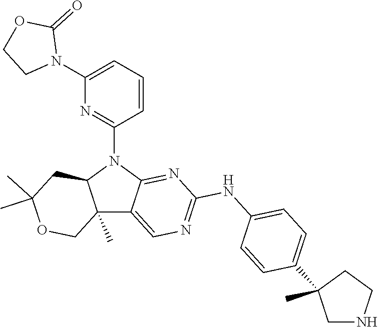 [SMILES: CC1(C)C[C@H]2N(c3cccc(N4CCOC4=O)n3)c3nc(Nc4ccc([C@]5(C)CCNC5)cc4)ncc3[C@@]2(C)CO1] 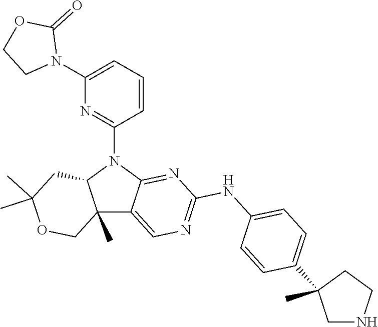 [SMILES: CC1(C)C[C@@H]2N(c3cccc(N4CCOC4=O)n3)c3nc(Nc4ccc([C@]5(C)CCNC5)cc4)ncc3[C@]2(C)CO1] | 664, 665 | 1H NMR (400 MHz, DMSO-d6) δ 9.35 (s, 1H), 8.41 (d, J = 8.0 Hz, 1H), 8.04 (d, J = 2.8 Hz, 1H), 7.87-7.78 (m, 1H), 7.72- 7.61 (m, 3H), 7.23 (d, J = 8.4 Hz, 2H), 4.60-4.56 (m, 1H), 4.52-4.41 (m, 2H), 4.33-4.23 (m, 1H), 4.22-4.13 (m, 1H), 3.83-3.63 (m, 2H), 3.52-3.38 (m, 1H), 3.09-3.00 (m, 1H), 2.98-2.88 (m, 2H), 2.33-2.23 (m, 1H), 2.04-1.80 (m, 2H), 1.63-1.53 (m, 1H), 1.28 (s, 3H), 1.26 (s, 3H), 1.24 (s, 3H), 1.05 (s, 3H). 1H NMR (400 MHz, DMSO-d6) δ 9.31 (s, 1H), 8.41 (d, J = 8.0 Hz, 1H), 8.04 (d, J = 2.8 Hz, 1H), 7.87-7.78 (m, 1H), 7.72- 7.61 (m, 3H), 7.23 (d, J = 8.4 Hz, 2H), 4.64-4.56 (m, 1H), 4.52-4.41 (m, 2H), 4.33-4.23 (m, 1H), 4.22-4.13 (m, 1H), 3.83-3.63 (m, 2H), 3.52-3.38 (m, 1H), 3.11-3.00 (m, 1H), 2.98-2.88 (m, 2H), 2.33-2.23 (m, 1H), 2.04-1.80 (m, 2H), 1.63-1.53 (m, 1H), 1.29 (s, 3H), 1.27 (s, 3H), 1.25 (s, 3H), 1.05 (s, 3H). | LCMS (ES, m/z): 556 [M + H]+, Rt 0.642 min LCMS (ES, m/z): 556 [M + H]+, Rt 0.650 min |
| 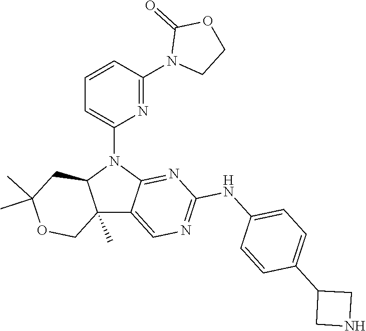 [SMILES: CC1(C)C[C@H]2N(c3cccc(N4CCOC4=O)n3)c3nc(Nc4ccc(C5CNC5)cc4)ncc3[C@@]2(C)CO1] 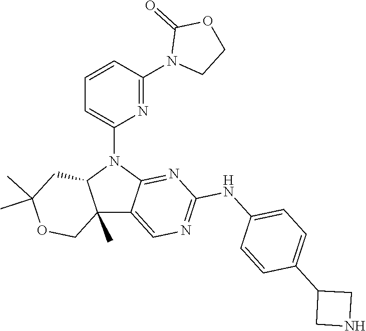 [SMILES: CC1(C)C[C@@H]2N(c3cccc(N4CCOC4=O)n3)c3nc(Nc4ccc(C5CNC5)cc4)ncc3[C@]2(C)CO1] | 666, 667 | 1H NMR (300 MHz, DMSO-d6) δ 9.35 (s, 1H), 8.41 (d, J = 8.1 Hz, 1H), 8.05 (s, 1H), 7.83 (d, J = 8.1 Hz, 1H), 7.75-7.64 (m, 3H), 7.27 (d, J = 8.4 Hz, 2H), 4.65- 4.42 (m, 3H), 4.32-4.11 (m, 2H), 3.89- 3.71 (m, 3H), 3.70-3.59 (m, 3H), 3.41- 3.35 (m, 1H), 2.32-2.26 (m, 1H), 1.63- 1.55 (m, 1H), 1.29 (s, 3H), 1.25 (s, 3H), 1.05 (s, 3H). 1H NMR (300 MHz, DMSO-d6) δ 9.36 (s, 1H), 8.42 (d, J = 8.1 Hz, 1H), 8.05 (s, 1H), 7.82 (d, J = 8.1 Hz, 1H), 7.75-7.64 (m, 3H), 7.27 (d, J = 8.1 Hz, 2H), 4.65- 4.42 (m, 3H), 4.35-4.11 (m, 3H), 3.87- 3.61 (m, 6H), 2.32-2.26 (m, 1H), 1.63- 1.55 (m, 1H), 1.29 (s, 3H), 1.25 (s, 3H), 1.06 (s, 3H). | LCMS (ES, m/z): 528 [M + H]+, Rt 0.649 min. LCMS (ES, m/z): 528 [M + H]+, Rt 0.638 min. |
| 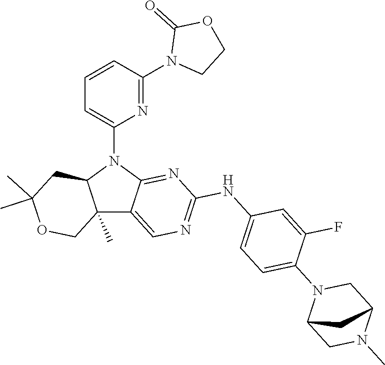 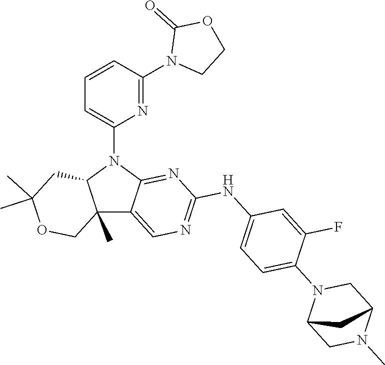 | 668, 669 | 1H NMR (300 MHz, DMSO-d6) δ 9.23 (s, 1H), 8.38 (d, J = 8.1 Hz, 1H), 8.02 (s, 1H), 7.84-7.74 (m, 1H), 7.73-7.67 (m, 1H), 7.67-7.57 (m, 1H), 7.27-7.19 (m, 1H), 6.76-6.63 (m, 1H), 4.64-4.54 (m, 1H), 4.52-4.42 (m, 2H), 4.35-4.11 (m, 3H), 3.83-3.61 (m, 2H), 3.47-3.34 (m, 2H), 3.25-3.15 (m, 1H), 2.79-2.61(m, 2H), 2.32-2.22 (m, 4H), 1.86-1.68 (m, 2H), 1.64-1.52 (m, 1H), 1.28 (s, 3H), 1.24 (s, 3H), 1.05 (s, 3H). 1H NMR (300 MHz, DMSO-d6) δ 9.23 (s, 1H), 8.38 (d, J = 8.1 Hz, 1H), 8.02 (s, 1H), 7.84-7.74 (m, 1H), 7.73-7.57 (m, 2H), 7.27-7.19 (m, 1H), 6.76-6.63 (m, 1H), 4.64-4.54 (m, 1H), 4.52-4.42 (m, 2H), 4.35-4.11 (m, 3H), 3.83-3.61 (m, 2H), 3.47-3.34 (m, 2H), 3.25-3.15 (m, 1H), 2.79-2.61(m, 2H), 2.32-2.22 (m, 4H), 1.86-1.68 (m, 2H), 1.64-1.52 (m, 1H), 1.28 (s, 3H), 1.24 (s, 3H), 1.05 (s, 3H) | LCMS (ES, m/z): 601 [M + H]+, Rt 0.650 min. LCMS (ES, m/z): 601 [M + H]+, Rt 0.650 min. |
| 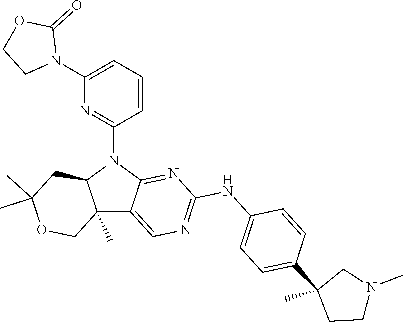 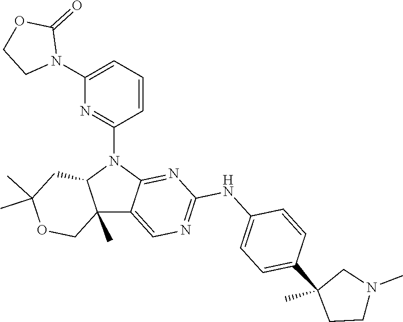 | 670, 671 | 1H NMR (400 MHz, DMSO-d6) δ 9.29 (s, 1H), 8.42 (d, J = 8.0 Hz, 1H), 8.03 (s, 1H), 7.85-7.77 (m, 1H), 7.69 (d, J = 8.0 Hz, 1H), 7.67-7.60 (m, 2H), 7.26-7.20 (m, 2H), 4.64-4.55 (m, 1H), 4.53-4.41 (m, 2H), 4.33-4.23 (m, 1H), 4.23-4.13 (m, 1H), 3.83-3.63 (m, 2H), 2.83-2.73 (m, 1H), 2.67 (d, J = 8.8 Hz, 1H), 2.61 (d, J = 8.8 Hz, 1H), 2.49-2.42 (m, 1H), 2.33- 2.23 (m, 4H), 2.15-2.05 (m, 1H), 1.93- 1.82 (m, 1H), 1.64-1.54 (m, 1H), 1.36 (s, 3H), 1.28 (s, 3H), 1.25 (s, 3H), 1.05 (s, 3H). 1H NMR (400 MHz, DMSO-d6) δ 9.29 (s, 1H), 8.42 (d, J = 8.0 Hz, 1H), 8.03 (s, 1H), 7.85-7.77 (m, 1H), 7.69 (d, J = 8.0 Hz, 1H), 7.66-7.60 (m, 2H), 7.26-7.20 (m, 2H), 4.64-4.56 (m, 1H), 4.52-4.42 (m, 2H), 4.33-4.23 (m, 1H), 4.23-4.13 (m, 1H), 3.83-3.63 (m, 2H), 2.83-2.73 (m, 1H), 2.67 (d, J = 8.8 Hz, 1H), 2.62 (d, J = 8.8 Hz, 1H), 2.49-2.42 (m, 1H), 2.34- 2.23 (m, 4H), 2.15-2.05 (m, 1H), 1.93- 1.82 (m, 1H), 1.64-1.54 (m, 1H), 1.36 (s, 3H), 1.28 (s, 3H), 1.25 (s, 3H), 1.05 (s, 3H). | LCMS (ES, m/z): 570 [M + H]+, Rt 0.650 min LCMS (ES, m/z): 570 [M + H]+, Rt 0.642 min |
| 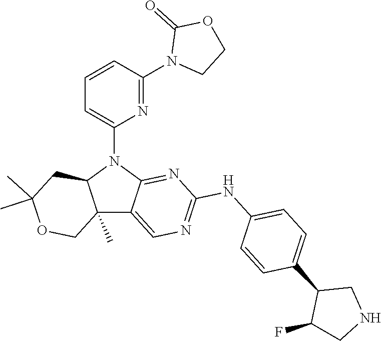 [SMILES: CC1(C)C[C@H]2N(c3cccc(N4CCOC4=O)n3)c3nc(Nc4ccc([C@H]5CNC[C@H]5F)cc4)ncc3[C@@]2(C)CO1] 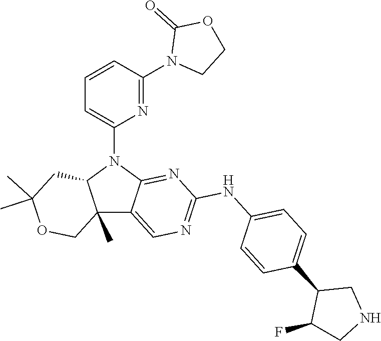 [SMILES: CC1(C)C[C@@H]2N(c3cccc(N4CCOC4=O)n3)c3nc(Nc4ccc([C@H]5CNC[C@H]5F)cc4)ncc3[C@]2(C)CO1] | 672, 673 | 1H NMR (400 MHz, DMSO-d6) δ 9.35 (s, 1H), 8.42 (d, J = 8.4 Hz, 1H), 8.04 (s, 1H), 7.83 (t, J = 8.0 Hz, 1H), 7.76-7.65 (m, 3H), 7.19 (d, J = 8.4 Hz, 2H), 5.19- 5.03 (m, 1H), 4.62-4.45 (m, 3H), 4.31- 4.15 (m, 2H), 3.81-3.62 (m, 2H), 3.42- 3.37 (m, 2H), 3.24-3.04 (m, 3H), 2.73- 2.67 (m, 1H), 2.32-2.26 (m, 1H), 1.62- 1.57 (m, 1H), 1.29 (s, 3H), 1.25 (s, 3H), 1.05 (s, 3H). 1H NMR (400 MHz, DMSO-d6) δ 9.36 (s, 1H), 8.42 (d, J = 8.4 Hz, 1H), 8.04 (s, 1H), 7.83 (t, J = 8.0 Hz, 1H), 7.75-7.66 (m, 3H), 7.19 (d, J = 8.4 Hz, 2H), 5.19- 5.03 (m, 1H), 4.62-4.59 (m, 1H), 4.50- 4.45 (m, 2H), 4.31-4.15 (m, 2H), 3.81- 3.62 (m, 2H), 3.42-3.33 (m, 2H), 3.24- 3.19 (m, 1H), 3.13-3.12 (m, 1H), 3.06- 3.04 (m, 1H), 2.73-2.67 (m, 1H), 2.33- 2.26 (m, 1H), 1.62-1.57 (m, 1H), 1.29 (s, 3H), 1.24 (s, 3H), 1.05 (s, 3H). | LCMS (ES, m/z): 560 [M + H]+. Rt 0.630 min. LCMS (ES, m/z): 560 [M + H]+. Rt 0.622 min. |
| 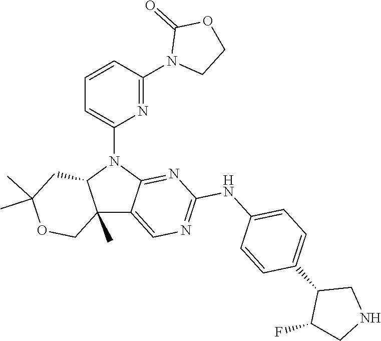 [SMILES: CC1(C)C[C@@H]2N(c3cccc(N4CCOC4=O)n3)c3nc(Nc4ccc([C@@H]5CNC[C@@H]5F)cc4)ncc3[C@]2(C)CO1] 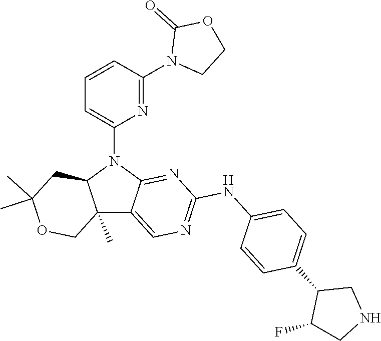 [SMILES: CC1(C)C[C@H]2N(c3cccc(N4CCOC4=O)n3)c3nc(Nc4ccc([C@@H]5CNC[C@@H]5F)cc4)ncc3[C@@]2(C)CO1] | 674, 675 | 1H NMR (300 MHz, DMSO-d6) δ 9.35 (s, 1H), 8.42 (d, J = 8.1 Hz, 1H), 8.04 (s, 1H), 7.83 (t, J = 8.1 Hz, 1H), 7.70-7.66 (m, 3H), 7.19 (d, J = 8.4 Hz, 2H), 5.20- 5.00 (m, 1H), 4.62-4.57 (m, 1H), 4.50- 4.44 (m, 2H), 4.29-4.16 (m, 2H), 3.81- 3.77 (m, 1H), 3.68-3.64 (m, 1H), 3.50- 3.32 (m, 2H), 3.13-3.02 (m, 2H), 2.72- 2.67 (m, 1H), 2.31-2.27 (m, 1H), 1.63- 1.55 (m, 1H), 1.29 (s, 3H), 1.25 (s, 3H), 1.05 (s, 3H). 1H NMR (300 MHz, DMSO-d6) δ 9.35 (s, 1H), 8.42 (d, J = 8.1 Hz, 1H), 8.04 (s, 1H), 7.83 (t, J = 8.1 Hz, 1H), 7.70-7.66 (m, 3H), 7.19 (d, J = 8.4 Hz, 2H), 5.20- 5.01 (m, 1H), 4.62-4.57 (m, 1H), 4.50- 4.44 (m, 2H), 4.28-4.16 (m, 2H), 3.81- 3.77 (m, 1H), 3.68-3.65 (m, 1H), 3.42- 3.32 (m, 2H), 3.13-3.02 (m, 2H), 2.72- 2.67 (m, 1H), 2.31-2.25 (m, 1H), 1.63- 1.58 (m, 1H), 1.28 (s, 3H), 1.24 (s, 3H), 1.05 (s, 3H). | LCMS (ES, m/z): 560 [M + H]+. Rt 0.627 min. LCMS (ES, m/z): 560.25 [M + H]+. Rt 0.626 min. |
| 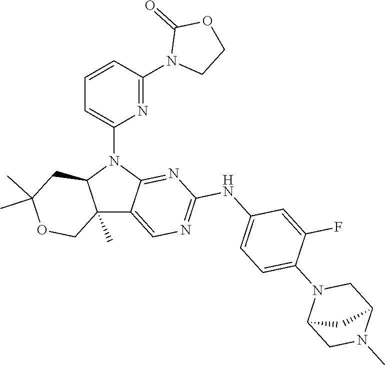 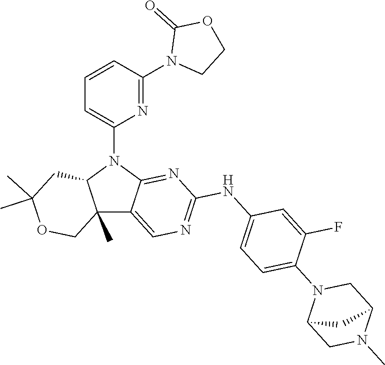 | 676, 677 | 1H NMR (300 MHz, DMSO-d6) δ 9.23 (s, 1H), 8.38 (d, J = 8.1 Hz, 1H), 8.02 (s, 1H), 7.82-7.61 (m, 3H), 7.25-7.22 (m, 1H), 6.74-6.58 (m, 1H), 4.98-4.58 (m, 1H), 4.50-4.44 (m, 2H), 4.29-4.16 (m, 3H), 3.80-3.64 (m, 2H), 3.41-3.32 (m, 2H), 3.28-3.21 (m, 1H), 2.85- 2.60 (m, 2H), 2.28-2.24 (m, 4H), 1.87-1.68 (m, 2H), 1.60-1.53 (m, 1H), 1.28 (s, 3H), 1.24 (s, 3H), 1.05 (s, 3H). 1H NMR (300 MHz, DMSO-d6) δ 9.23 (s, 1H), 8.38 (d, J = 8.1 Hz, 1H), 8.02 (s, 1H), 7.79 (t, J = 8.1 Hz, 1H), 7.70-7.60 (m, 2H), 7.25-7.21 (m, 1H), 6.69 (t, J = 9.7 Hz, 1H), 4.61-4.58 (m, 1H), 4.50- 4.44 (m, 2H), 4.29-4.16 (m, 3H), 3.80- 3.75 (m, 1H), 3.68-3.64 (m, 1H), 3.41- 3.32 (m, 2H), 3.28-3.21 (m, 1H), 2.85- 2.60 (m, 2H), 2.28-2.24 (m, 4H), 1.82- 1.75 (m, 1H), 1.74-1.68 (m, 1H), 1.60- 1.53 (m, 1H), 1.28 (s, 3H), 1.24 (s, 3H), 1.10- 0.89 (m, 3H). | LCMS (ES, m/z): 601 [M + H]+, Rt 0.640 min. LCMS (ES, m/z): 601 [M + H]+, Rt 0.639 min. |
| 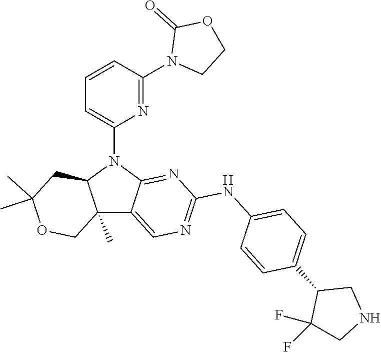 [SMILES: CC1(C)C[C@H]2N(c3cccc(N4CCOC4=O)n3)c3nc(Nc4ccc([C@@H]5CNCC5(F)F)cc4)ncc3[C@@]2(C)CO1] 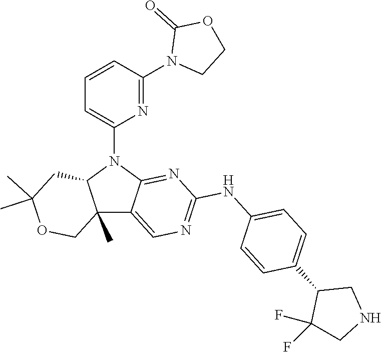 [SMILES: CC1(C)C[C@@H]2N(c3cccc(N4CCOC4=O)n3)c3nc(Nc4ccc([C@@H]5CNCC5(F)F)cc4)ncc3[C@]2(C)CO1] | 680, 681 | 1H NMR (300 MHz, DMSO-d6) δ 9.39 (s, 1H), 8.42 (d, J = 8.1 Hz, 1H), 8.05 (s, 1H), 7.82 (t, J = 8.1 Hz, 1H), 7.72-7.68 (m, 3H), 7.23 (d, J = 8.7 Hz, 2H), 4.63- 4.58 (m, 1H), 4.53-4.41 (m, 2H), 4.32- 4.15 (m, 2H), 3.82-3.74 (m, 1H), 3.73- 3.64 (m, 1H), 3.51-3.39 (m, 3H), 3.21- 3.06 (m, 2H), 2.32-2.25 (m, 1H), 1.64- 1.56 (m, 1H), 1.29 (s, 3H), 1.25 (s, 3H), 1.05 (s, 3H). 1H NMR (300 MHz, DMSO-d6) δ 9.39 (s, 1H), 8.42 (d, J = 8.1 Hz, 1H), 8.05 (s, 1H), 7.82 (t, J = 8.1 Hz, 1H), 7.72-7.68 (m, 3H), 7.23 (d, J = 8.7 Hz, 2H), 4.63- 4.58 (m, 1H), 4.53-4.41 (m, 2H), 4.32- 4.15 (m, 2H), 3.82-3.74 (m, 1H), 3.73- 3.65 (m, 1H), 3.51-3.39 (m, 3H), 3.21- 3.08 (m, 2H), 2.31-2.25 (m, 1H), 1.64- 1.56 (m, 1H), 1.29 (s, 3H), 1.25 (s, 3H), 1.05 (s, 3H). | LCMS (ES, m/z): 578 [M + H]+. Rt 0.637 min. LCMS (ES, m/z): 578 [M + H]+. Rt 0.632 min. |
| 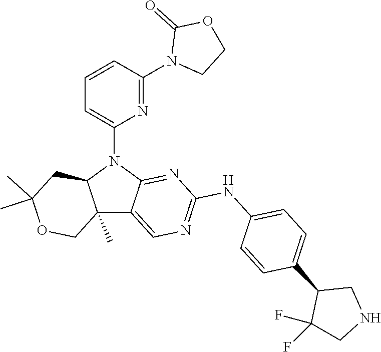 [SMILES: CC1(C)C[C@H]2N(c3cccc(N4CCOC4=O)n3)c3nc(Nc4ccc([C@H]5CNCC5(F)F)cc4)ncc3[C@@]2(C)CO1] 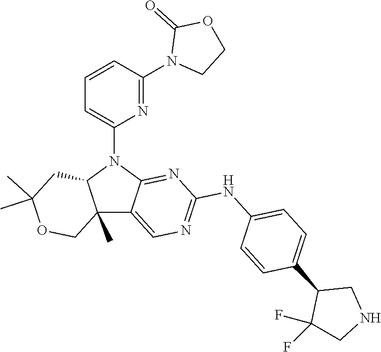 [SMILES: CC1(C)C[C@@H]2N(c3cccc(N4CCOC4=O)n3)c3nc(Nc4ccc([C@H]5CNCC5(F)F)cc4)ncc3[C@]2(C)CO1] | 682, 683 | 1H NMR (400 MHz, DMSO-d6) δ 9.39 (s, 1H), 8.42 (d, J = 8.0 Hz, 1H), 8.05 (s, 1H), 7.82 (t, J = 8.4 Hz, 1H), 7.71-7.69 (m, 3H), 7.23 (d, J = 8.4 Hz, 2H), 4.62- 4.59 (m, 1H), 4.50-4.45 (m, 2H), 4.31- 4.15 (m, 2H), 3.81-3.65 (m, 2H), 3.46- 3.36 (m, 3H), 3.19-3.07 (m, 2H), 2.33- 2.26 (m, 1H), 1.63-1.57 (m, 1H), 1.29 (s, 3H), 1.25 (s, 3H), 1.05 (s, 3H). 1H NMR (400 MHz, DMSO-d6) δ 9.39 (s, 1H), 8.42 (d, J = 8.4 Hz, 1H), 8.05 (s, 1H), 7.84-7.69 (m, 4H), 7.22 (d, J = 8.4 Hz, 2H), 4.62-4.59 (m, 1H), 4.50-4.45 (m, 2H), 4.31-4.15 (m, 2H), 3.83-3.65 (m, 2H), 3.46-3.36 (m, 3H), 3.19-3.07 (m, 2H), 2.32-2.26 (m, 1H), 1.63-1.57 (m, 1H), 1.29 (s, 3H), 1.25 (s, 3H), 1.05 (s, 3H). | LCMS (ES, m/z): 578 [M + H]+. Rt 0.638 min. LCMS (ES, m/z): 578 [M + H]+. Rt 0.641 min. |
| 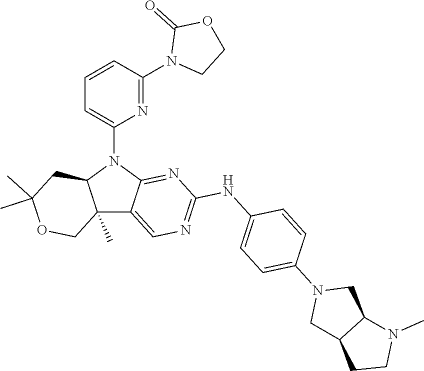 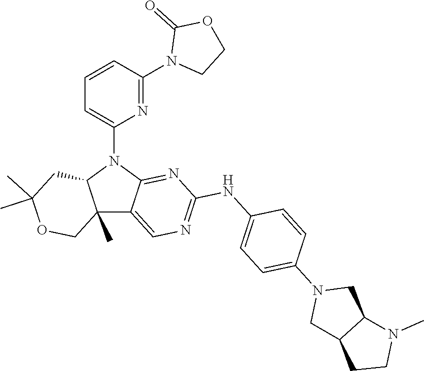 | 684, 685 | 1H NMR (400 MHz, DMSO-d6) δ 9.00 (s, 1H), 8.40 (d, J = 8.0 Hz, 1H), 7.97 (s, 1H), 7.83-7.75 (m, 1H), 7.67 (d, J = 8.4 Hz, 1H), 7.46 (d, J = 8.4 Hz, 2H), 6.61 (d, J = 8.8 Hz, 2H), 4.61-4.54 (m, 1H), 4.51- 4.40 (m, 2H), 4.32-4.22 (m, 1H), 4.22- 4.12 (m, 1H), 3.81-3.62 (m, 1H), 3.34- 3.28 (m, 1H), 3.27-3.20 (m, 2H), 3.14- 3.02 (m, 2H), 3.00-2.92 (m, 1H), 2.91- 2.80 (m, 2H), 2.33-2.19 (m, 5H), 2.09- 1.97 (m, 1H), 1.65-1.52 (m, 2H), 1.27 (s, 3H), 1.24 (s, 3H), 1.05 (s, 3H). 1H NMR (400 MHz, DMSO-d6) δ 9.00 (s, 1H), 8.40 (d, J = 8.0 Hz, 1H), 7.97 (s, 1H), 7.83-7.75 (m, 1H), 7.67 (d, J = 8.0 Hz, 1H), 7.46 (d, J = 8.8 Hz, 2H), 6.61 (d, J = 8.8 Hz, 2H), 4.61-4.54 (m, 1H), 4.51- 4.41 (m, 2H), 4.32-4.22 (m, 1H), 4.22- 4.12 (m, 1H), 3.81-3.62 (m, 1H), 3.34- 3.28 (m, 1H), 3.27-3.19 (m, 2H), 3.14- 3.02 (m, 2H), 3.01-2.94 (m, 1H), 2.91- 2.84 (m, 2H), 2.33-2.23 (m, 5H), 2.09- 1.99 (m, 1H), 1.65-1.52 (m, 2H), 1.27 (s, 3H), 1.24 (s, 3H), 1.05 (s, 3H). | LCMS (ES, m/z): 597 [M + H]+, Rt 0.642 min. LCMS (ES, m/z): 597 [M + H]+, Rt 0.642 min. |
| 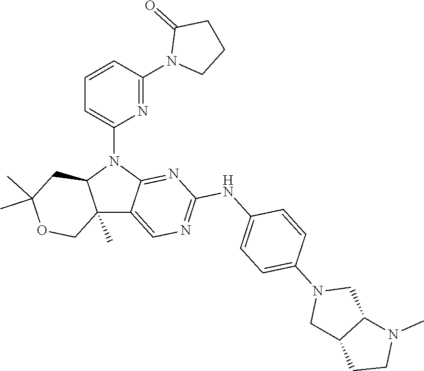 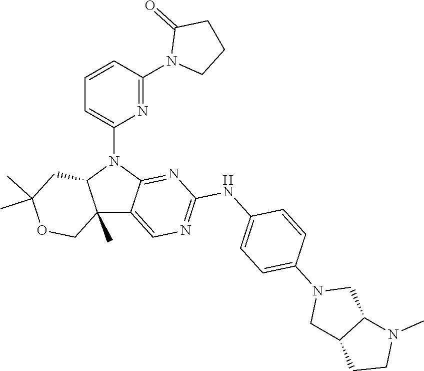 | 686, 687 | 1H NMR (300 MHz, DMSO-d6) δ 8.98 (s, 1H), 8.39 (d, J = 8.1 Hz, 1H), 8.12-7.87 (m, 2H), 7.76 (t, J = 8.1 Hz, 1H), 7.65- 7.32 (m, 2H), 6.57 (d, J = 8.7 Hz, 2H), 4.75-4.35 (m, 1H), 4.25-3.88 (m, 2H), 3.82-3.55 (m, 2H), 3.32-3.19 (m, 2H), 3.18-3.02 (m, 2H), 3.02-2.83 (m, 3H), 2.65-2.55 (m, 2H), 2.32-2.22 (m, 5H), 2.09-2.00 (m, 3H), 1.70-1.50 (m, 2H), 1.27 (s, 3H), 1.24 (s, 3H), 1.05 (s, 3H). 1H NMR (300 MHz, DMSO-d6) δ 8.98 (s, 1H), 8.39 (d, J = 8.1 Hz, 1H), 8.12-7.87 (m, 2H), 7.76 (t, J = 8.1 Hz, 1H), 7.65- 7.32 (m, 2H), 6.57 (d, J = 8.7 Hz, 2H), 4.75-4.35 (m, 1H), 4.25-4.04 (m, 1H), 4.04-3.88 (m, 1H), 3.82-3.55 (m, 2H), 3.32-3.19 (m, 2H), 3.18-3.02 (m, 2H), 3.02- 2.83 (m, 3H), 2.65-2.55 (m, 2H), 2.32-2.22 (m, 5H), 2.09-2.00 (m, 3H), 1.70-1.50 (m, 2H), 1.27 (s, 3H), 1.24 (s, 3H), 1.05 (s, 3H). | LCMS(ES, m/z): 595 [M + H]+. Rt 0.672 min. LCMS(ES, m/z): 595 [M + H]+. Rt 0.677 min. |
| 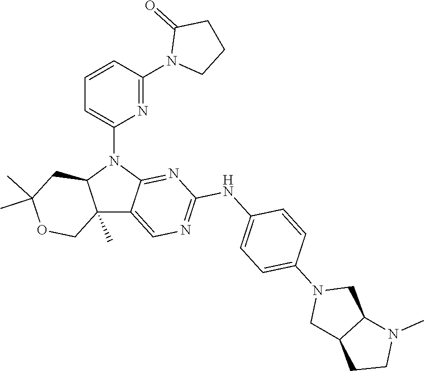 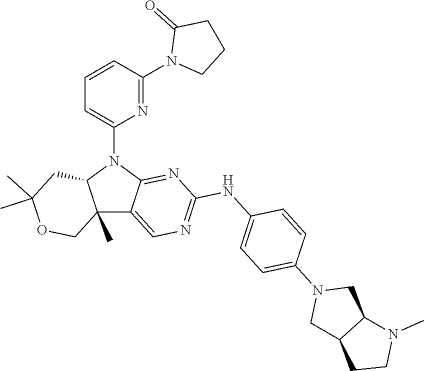 | 688, 689 | 1H NMR (400 MHz, DMSO-d6) δ 8.99 (s, 1H), 8.39 (d, J = 8.0 Hz, 1H), 7.96 (s, 1H), 7.94-7.87 (m, 1H), 7.81-7.72 (m, 1H), 7.46 (d, J = 8.4 Hz, 2H), 6.57 (d, J = 8.8 Hz, 2H), 4.61-4.53 (m, 1H), 4.15- 4.05 (m, 1H), 4.02-3.91 (m, 1H), 3.80- 3.62 (m, 2H), 3.34-3.27 (m, 1H), 3.27- 3.21 (m, 1H), 3.14-3.02 (m, 2H), 2.99- 2.92 (m, 1H), 2.91-2.82 (m, 2H), 2.63- 2.54 (m, 2H), 2.34-2.19 (m, 5H), 2.11- 1.99 (m, 3H), 1.65-1.52 (m, 2H), 1.27 (s, 3H), 1.24 (s, 3H), 1.05 (s, 3H). 1H NMR (400 MHz, DMSO-d6) δ 8.98 (s, 1H), 8.39 (d, J = 8.0 Hz, 1H), 7.96 (s, 1H), 7.93-7.88 (m, 1H), 7.80-7.72 (m, 1H), 7.46 (d, J = 8.4 Hz, 2H), 6.57 (d, J = 8.8 Hz, 2H), 4.61-4.53 (m, 1H), 4.15- 4.05 (m, 1H), 4.02-3.91 (m, 1H), 3.80- 3.62 (m, 2H), 3.34-3.27 (m, 1H), 3.27- 3.20 (m, 1H), 3.14-3.02 (m, 2H), 2.99- 2.92 (m, 1H), 2.90-2.80 (m, 2H), 2.63- 2.54 (m, 2H), 2.34-2.19 (m, 5H), 2.11- 1.99 (m, 3H), 1.65-1.52 (m, 2H), 1.27 (s, 3H), 1.24 (s, 3H), 1.05 (s, 3H). | LCMS (ES, m/z): 595 [M + H]+, Rt 0.667 min LCMS (ES, m/z): 595 [M + H]+, Rt 0.667 min |
| 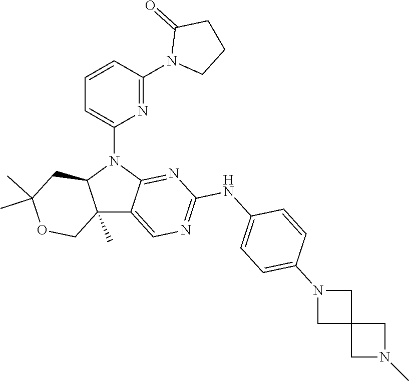 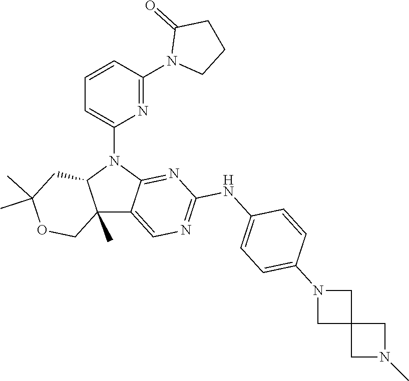 | 690, 691 | 1H NMR (300 MHz, DMSO-d6) δ 9.00 (s, 1H), 8.37 (d, J = 8.1 Hz, 1H), 7.95-7.89 (m, 2H), 7.75 (t, J = 8.1 Hz, 1H), 7.45 (d, J = 8.7 Hz, 2H), 6.39 (d, J = 9.0 Hz, 2H), 4.59-4.55 (m, 1H), 4.12-4.08 (m, 1H), 3.98-3.94 (m, 1H), 3.80-3.74 (m, 5H), 3.67-3.63 (m, 1H), 3.32-3.26 (m, 4H), 2.58-2.51 (m, 2H), 2.29-2.24 (m, 1H), 2.19 (s, 3H), 2.10-2.0 (m, 2H), 1.60-1.53 (m, 1H), 1.27 (s, 3H), 1.24 (s, 3H), 1.05 (s, 3H). 1H NMR (300 MHz, DMSO-d6) δ 9.00 (s, 1H), 8.37 (d, J = 8.1 Hz, 1H), 7.95-7.89 (m, 2H), 7.75 (t, J = 8.1 Hz, 1H), 7.45 (d, J = 8.7 Hz, 2H), 6.39 (d, J = 9.0 Hz, 2H), 4.59-4.55 (m, 1H), 4.12-4.08 (m, 1H), 3.98-3.94 (m, 1H), 3.80-3.74 (m, 5H), 3.67-3.63 (m, 1H), 3.32-3.26 (m, 4H), 2.58-2.51 (m, 2H), 2.29-2.24 (m, 1H), 2.19 (s, 3H), 2.10-2.0 (m, 2H), 1.60-1.53 (m, 1H), 1.27 (s, 3H), 1.24 (s, 3H), 1.05 (s, 3H). | LCMS (ES, m/z): 581 [M + H]+, Rt 0.639 min. LCMS (ES, m/z): 581 [M + H]+, Rt 0.640 min. |
| 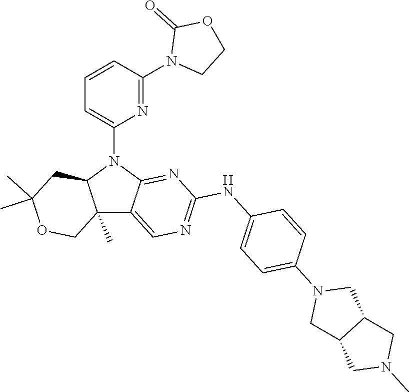 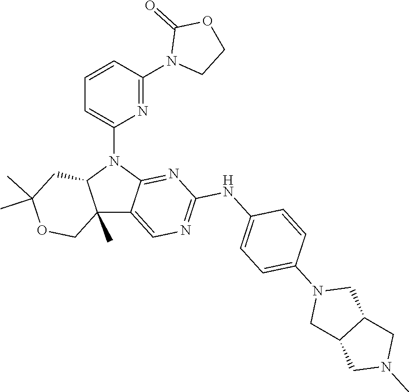 | 692, 693 | 1H NMR (400 MHz, DMSO-d6) δ 9.02 (s, 1H), 8.41 (d, J = 8.0 Hz, 1H), 7.97 (s, 1H), 7.82 (t, J = 8.0 Hz, 1H), 7.68 (d, J = 7.6 Hz, 1H), 7.49 (d, J = 8.8 Hz, 2H), 6.64 (d, J = 8.8 Hz, 2H), 4.59-4.56 (m, 1H), 4.49-4.45 (m, 2H), 4.31-4.24 (m, 1H), 4.21-4.14 (m, 1H), 3.81-3.65 (m, 2H) 3.29-3.28 (m, 2H), 3.05-3.02 (m, 2H), 2.09-2.83 (m, 2H), 2.61-2.57 (m, 2H), 2.39-2.36 (m, 2H), 2.30-2.22 (m, 4H), 1.60-1.54 (m, 1H), 1.27 (s, 3H), 1.24 (s, 3H), 1.05 (s, 3H). 1H NMR (400 MHz, DMSO-d6) δ 9.03 (s, 1H), 8.41 (d, J = 8.0 Hz, 1H), 7.97 (s, 1H), 7.82 (t, J = 8.0 Hz, 1H), 7.68 (d, J = 8.0 Hz, 1H), 7.49 (d, J = 8.8 Hz, 2H), 6.64 (d, J = 8.8 Hz, 2H), 4.59-4.56 (m, 1H), 4.49-4.45 (m, 2H), 4.31-4.24 (m, 1H), 4.21-4.14 (m, 1H), 3.81-3.65 (m, 2H) 3.33-3.28 (m, 2H), 3.05-3.02 (m, 2H), 2.91-2.83 (m, 2H), 2.62-2.59 (m, 2H), 2.40-2.37 (m, 2H), 2.30-2.23 (m, 4H), 1.60-1.54 (m, 1H), 1.27 (s, 3H), 1.24 (s, 3H), 1.05 (s, 3H). | LCMS (ES, m/z): 597 [M + H]+, Rt 0.647 min. LCMS (ES, m/z): 597 [M + H]+, Rt 0.647 min. |
| 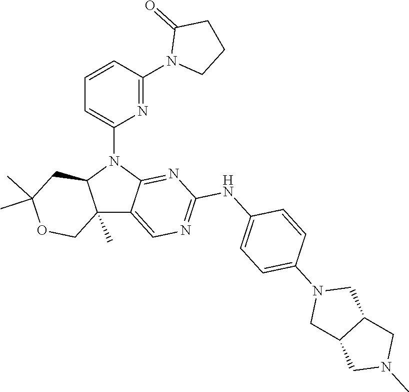 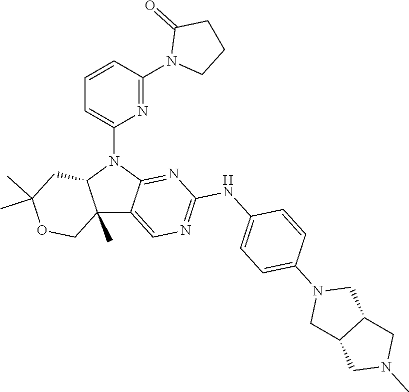 | 694, 695 | 1H NMR (300 MHz, DMSO-d6) δ 9.01 (s, 1H), 8.40 (d, J = 7.8 Hz, 1H), 7.96-7.89 (m, 2H), 7.79-7.73 (m, 1H), 7.49-7.46 (m, 2H), 6.64 (d, J = 8.7 Hz, 2H), 4.60- 4.55 (m, 1H), 4.12-4.08 (m, 1H), 3.98- 3.95 (m, 1H), 3.78-3.63 (m, 2H), 3.06- 3.04 (m, 2H), 2.88-2.72 (m, 2H), 2.60- 2.55 (m, 6H), 2.47-2.41 (m, 2H), 2.29- 2.26 (m, 4H), 2.07-2.02 (m, 2H), 1.61- 1.53 (m, 1H), 1.27 (s, 3H), 1.24 (s, 3H), 1.05 (s, 3H). 1H NMR (300 MHz, DMSO-d6) δ 9.00 (s, 1H), 8.40 (d, J = 8.1 Hz, 1H), 7.96-7.89 (m, 2H), 7.79-7.73 (m, 1H), 7.49-7.46 (m, 2H), 6.63 (d, J = 9.0 Hz, 2H), 4.60- 4.55 (m, 1H), 4.12-4.08 (m, 1H), 3.98- 3.95 (m, 1H), 3.78-3.74 (m, 1H), 3.67- 3.63 (m, 1H), 3.32-3.27 (m, 2H), 3.04- 3.00 (m, 2H), 2.86-2.81 (m, 2H), 2.60- 2.55 (m, 4H), 2.38-2.31 (m, 2H), 2.29- 2.24 (m, 1H), 2.21 (s, 3H), 2.07-2.02 (m, 2H), 1.61-1.53 (m, 1H), 1.27 (s, 3H), 1.24 (s, 3H), 1.05 (s, 3H). | LCMS (ES, m/z): 595 [M + H]+. Rt 0.664 min. LCMS (ES, m/z): 595 [M + H]+. Rt 0.656 min. |
| 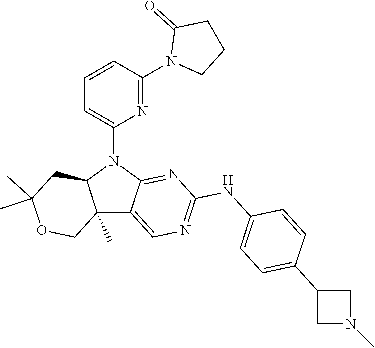 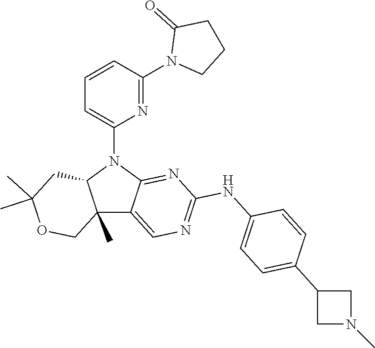 | 696, 697 | 1H NMR (400 MHz, DMSO-d6) δ 9.32 (s, 1H), 8.41 (d, J = 8.0 Hz, 1H), 8.03 (s, 1H), 7.93 (d, J = 8.4 Hz, 1H), 7.80 (t, J = 8.1 Hz, 1H), 7.67 (d, J = 8.4 Hz, 2H), 7.24 (d, J = 8.8 Hz, 2H), 4.62-4.59 (m, 1H), 4.12-4.00 (m, 1H), 3.99-3.96 (m, 1H), 3.87-3.76 (m, 1H), 3.72-3.65 (m, 1H), 3.61-3.58 (m, 2H), 3.52-3.49 (m, 1H), 3.03 (t, J = 6.8 Hz, 2H), 2.61-2.50 (m, 2H), 2.31-2.26 (m, 4H), 2.10-2.04 (m, 2H), 1.63-1.57 (m, 1H), 1.29 (s, 3H), 1.25 (s, 3H), 1.05 (s, 3H). 1H NMR (400 MHz, DMSO-d6) δ 9.32 (s, 1H), 8.40 (d, J = 8.0 Hz, 1H), 8.03 (s, 1H), 7.93 (d, J = 8.4 Hz, 1H), 7.80 (t, J = 8.1 Hz, 1H), 7.67 (d, J = 8.4 Hz, 2H), 7.24 (d, J = 8.8 Hz, 2H), 4.62-4.58 (m, 1H), 4.12-4.08 (m, 1H), 4.00-3.96 (m, 1H), 3.87-3.78 (m, 1H), 3.72-3.65 (m, 1H), 3.61-3.58 (m, 2H), 3.52-3.49 (m, 1H), 3.03 (t, J = 6.8 Hz, 2H), 2.60-2.50 (m, 2H), 2.31-2.26 (m, 4H), 2.10-2.04 (m, 2H), 1.63-1.57 (m, 1H), 1.29 (s, 3H), 1.25 (s, 3H), 1.05 (s, 3H). | LCMS (ES, m/z): 540 [M + H]+, Rt 1.183 min. LCMS (ES, m/z): 540 [M + H]+, Rt 1.190 min. |
| 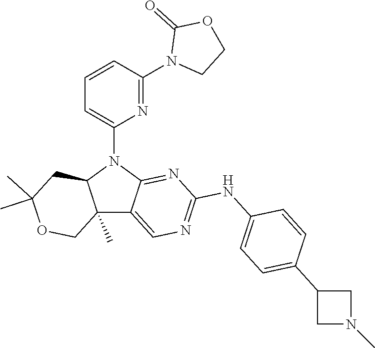 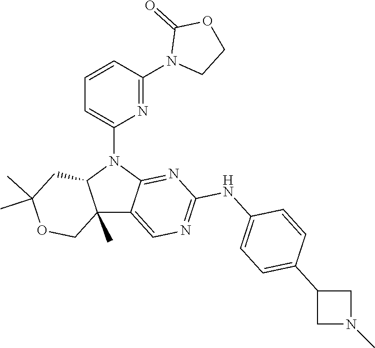 | 698, 699 | 1H NMR (300 MHz, DMSO-d6) δ 9.34 (s, 1H), 8.41 (d, J = 8.1 Hz, 1H), 8.04 (s, 1H), 7.83 (t, J = 8.1 Hz, 1H), 7.74-7.65 (m, 3H), 7.24 (d, J = 8.4 Hz, 2H), 4.61- 4.43 (m, 2H), 4.31-4.13 (m, 2H), 3.88- 3.57 (m, 4H), 3.56-3.45 (m, 2H), 3.09- 3.01 (m, 2H), 3.33-3.24 (m, 4H), 2.67- 2.50 (m, 1H), 1.29(s, 3H), 1.25 (s, 3H), 1.05 (s, 3H). 1H NMR (300 MHz, DMSO-d6) δ 9.34 (s, 1H), 8.41 (d, J = 8.1 Hz, 1H), 8.04 (s, 1H), 7.83 (t, J = 8.1 Hz, 1H), 7.74-7.65 (m, 3H), 7.24 (d, J = 8.4 Hz, 2H), 4.61- 4.43 (m, 2H), 4.31-4.13 (m, 2H), 3.88- 3.57 (m, 4H), 3.56-3.45 (m, 2H), 3.09- 3.01 (m, 2H), 3.33-3.24 (m, 4H), 2.67- 2.50 (m, 1H), 1.29 (s, 3H), 1.25 (s, 3H), 1.05 (s, 3H). | LCMS (ES, m/z): 542 [M + H]+, Rt 1.309 min. LCMS (ES, m/z): 542 [M + H]+, Rt 1.290 min. |
| 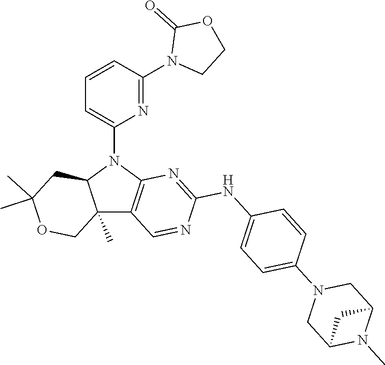 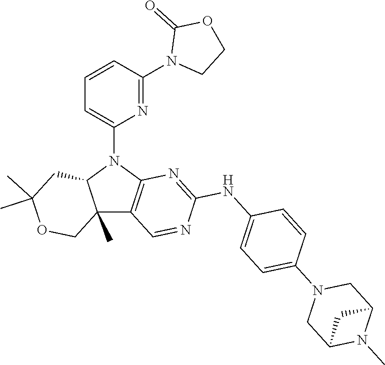 | 700, 701 | 1H NMR (300 MHz, DMSO-d6) δ 9.00 (s, 1H), 8.41 (d, J = 8.1 Hz, 1H), 7.97 (s, 1H), 7.80 (t, J = 8.1 Hz, 1H), 7.67 (d, J = 8.1 Hz, 1H), 7.51 (d, J = 9.0 Hz, 2H), 6.69 (d, J = 9.0 Hz, 2H), 4.60-4.41 (m, 3H), 4.35-4.12 (m, 2H), 3.85-3.61 (m, 2H), 3.57 (d, J = 7.6 Hz, 2H), 3.45-3.38 (m, 2H), 3.31-3.22 (m, 2H), 2.45-2.21 (m, 2H), 1.99 (s, 3H), 1.62-1.51 (m, 2H), 1.27 (s, 3H), 1.25 (s, 3H), 1.06 (s, 3H). 1H NMR (300 MHz, DMSO-d6) δ 9.00 (s, 1H), 8.42 (d, J = 8.1 Hz, 1H), 7.97 (s, 1H), 7.80 (t, J = 8.1 Hz, 1H), 7.67 (d, J = 8.1 Hz, 1H), 7.51 (d, J = 9.0 Hz, 2H), 6.70 (d, J = 9.0 Hz, 2H), 4.60-4.41 (m, 3H), 4.35-4.12 (m, 2H), 3.85-3.61 (m, 2H), 3.57 (d, J = 7.6 Hz, 2H), 3.45-3.38 (m, 2H), 3.31-3.22 (m, 2H), 2.45-2.21 (m, 2H), 1.99 (s, 3H), 1.62-1.51 (m, 2H), 1.27 (s, 3H), 1.25 (s, 3H), 1.06 (s, 3H). | LCMS (ES, m/z): 583 [M + H]+; RT: 0.628 min. LCMS (ES, m/z): 583 [M + H]+; RT: 0.635 min. |
| 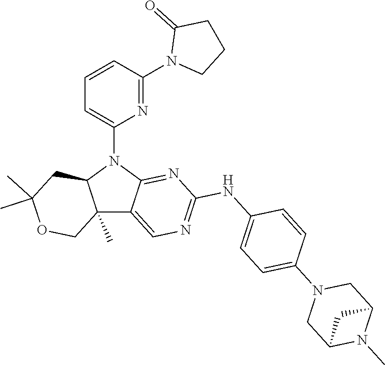 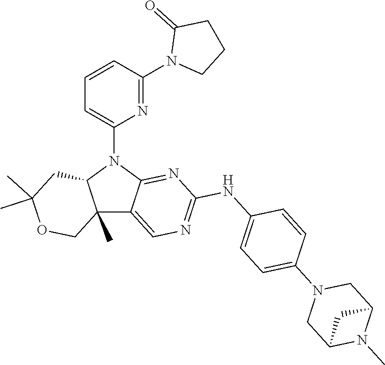 | 703, 704 | 1H NMR (300 MHz, DMSO-d6) δ 8.99 (s, 1H), 8.41 (d, J = 8.1 Hz, 1H), 7.99-7.86 (m, 2H), 7.77 (t, J = 8.1 Hz, 1H), 7.52 (d, J = 8.7 Hz, 2H), 6.69 (d, J = 9.0 Hz, 2H), 4.63-4.52 (m, 1H), 4.18-4.04 (m, 1H), 4.04-3.90 (m, 1H), 3.81-3.63 (m, 2H), 3.62-3.56 (m, 2H), 3.54-3.38 (m, 2H), 3.32-3.23 (m, 2H), 2.64-2.52 (m, 2H), 2.46-2.38 (m, 1H), 2.34-2.22 (m, 1H), 2.13-1.98 (m, 5H), 1.64-1.51 (m, 2H), 1.28 (s, 3H), 1.24 (s, 3H), 1.06 (s, 3H). 1H NMR (300 MHz, DMSO-d6) δ 8.99 (s, 1H), 8.41 (d, J = 8.1 Hz, 1H), 7.99-7.86 (m, 2H), 7.77 (t, J = 8.1 Hz, 1H), 7.52 (d, J = 8.7 Hz, 2H), 6.69 (d, J = 9.0 Hz, 2H), 4.63-4.52 (m, 1H), 4.18-4.04 (m, 1H), 4.04-3.85 (m, 1H), 3.81-3.62 (m, 2H), 3.61-3.54 (m, 2H), 3.47-3.38 (m, 2H), 3.31-3.21 (m, 2H), 2.67-2.52 (m, 2H), 2.46-2.38 (m, 1H), 2.34-2.22 (m, 1H), 2.13-2.01 (m, 2H), 1.99 (s, 3H), 1.64- 1.50 (m, 2H), 1.28 (s, 3H), 1.24 (s, 3H), 1.06 (s, 3H). | LCMS (ES, m/z): 581 [M + H]+; RT: 0.663 min. LCMS (ES, m/z): 581 [M + H]+; RT: 0.654 min. |
| 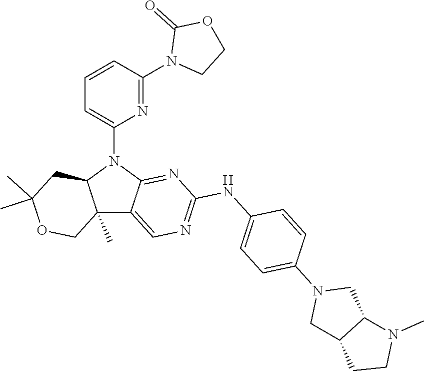 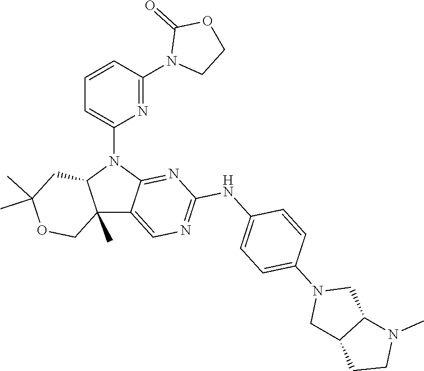 | 705, 706 | 1H NMR (300 MHz, DMSO-d6) δ 8.99 (s, 1H), 8.40 (d, J = 8.1 Hz, 1H), 7.97 (s, 1H), 7.79 (t, J = 8.1 Hz, 1H), 7.71-7.62 (m, 1H), 7.58-7.32 (m, 2H), 6.75-6.45 (m, 2H), 4.70-4.52 (m, 1H), 4.52-4.38 (m, 2H), 4.38-4.10 (m, 2H), 3.82-3.72 (m, 1H), 3.72-3.60 (m, 1H), 3.30-3.18 (m, 2H), 3.16-3.02 (m, 2H), 3.02- 2.75 (m, 3H), 2.33-2.22 (m, 5H), 2.12-1.96 (m, 1H), 1.65-1.50 (m, 2H), 1.27 (s, 3H), 1.24 (s, 3H), 1.05 (s, 3H). 1H NMR (300 MHz, DMSO-d6) δ 8.99 (s, 1H), 8.40 (d, J = 8.1 Hz, 1H), 7.97 (s, 1H), 7.79 (t, J = 8.1 Hz, 1H), 7.71-7.62 (m, 1H), 7.58-7.32 (m, 2H), 6.75-6.45 (m, 2H), 4.70-4.52 (m, 1H), 4.52-4.38 (m, 2H), 4.38-4.10 (m, 2H), 3.82-3.72 (m, 1H), 3.72-3.60 (m, 1H), 3.30-3.18 (m, 2H), 3.16-3.02 (m, 2H), 3.02- 2.75 (m, 3H), 2.33-2.22 (m, 5H), 2.12-1.96 (m, 1H), 1.65-1.50 (m, 2H), 1.27 (s, 3H), 1.24 (s, 3H), 1.05 (s, 3H). | LCMS(ES, m/z): 597 [M + H]+. Rt 0.642 min. LCMS(ES, m/z): 597 [M + H]+. Rt 0.643 min. |
| 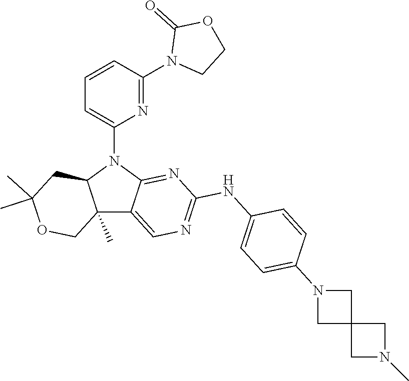 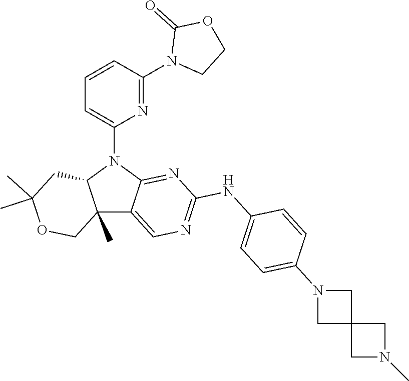 | 707, 708 | 1H NMR (300 MHz, DMSO-d6) δ 9.01 (s, 1H), 8.38 (d, J = 8.1 Hz, 1H), 7.96 (s, 1H), 7.78 (t, J = 8.1 Hz, 1H), 7.67 (d, J = 7.8 Hz, 1H), 7.45 (d, J = 8.7 Hz, 2H), 6.39 (d, J = 9.0 Hz, 2H), 4.59-4.54 (m, 1H), 4.49-4.44 (m, 2H), 4.28-4.15 (m, 2H), 3.80-3.63 (m, 6H), 3.32-3.25 (m, 4H), 2.30-2.24 (m, 1H), 2.18 (s, 3H), 1.60- 1.56 (m, 1H), 1.27 (s, 3H), 1.24 (s, 3H), 1.05 (s, 3H). 1H NMR (300 MHz, DMSO-d6) δ 9.01 (s, 1H), 8.38 (d, J = 8.1 Hz, 1H), 7.96 (s, 1H), 7.78 (t, J = 8.1 Hz, 1H), 7.67 (d, J = 7.8 Hz, 1H), 7.45 (d, J = 8.7 Hz, 2H), 6.39 (d, J = 9.0 Hz, 2H), 4.59-4.54 (m, 1H), 4.49-4.44 (m, 2H), 4.28-4.15 (m, 2H), 3.80-3.63 (m, 6H), 3.32-3.25 (m, 4H), 2.30-2.24 (m, 1H), 2.19 (s, 3H), 1.60- 1.56 (m, 1H), 1.26 (s, 3H), 1.24 (s, 3H), 1.04 (s, 3H). | LCMS (ES, m/z): 583 [M + H]+, Rt 0.620 min. LCMS (ES, m/z): 583 [M + H]+, Rt 0.622 min. |

### Examples 572 and 573. (4bR,8aR)-4b-methyl-9-(6-((methylamino)methyl)pyridin-2-yl)-N-(4-morpholinophenyl)-4b,5,7,8,8a,9-hexahydropyrano[3′,4′:4,5]pyrrolo[2,3-d]pyrimidin-2-amine and (4bS,8aS)-4b-methyl-9-(6-((methylamino)methyl)pyridin-2-yl)-N-(4-morpholinophenyl)-4b,5,7,8,8a,9-hexahydropyrano[3′,4′:4,5]pyrrolo[2,3-d]pyrimidin-2-amine

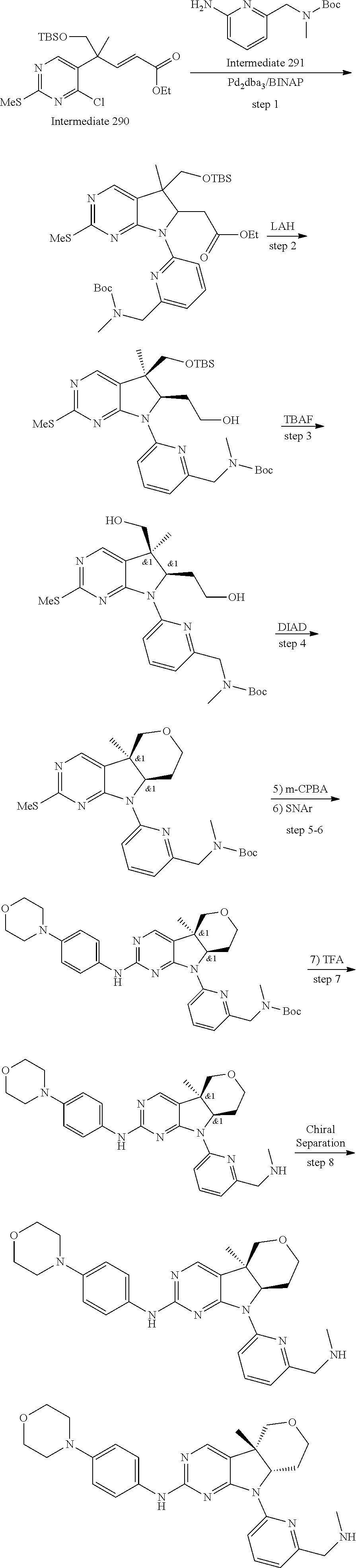

### Step 1. ethyl 2-(7-(6-(((tert-butoxycarbonyl)(methyl)amino)methyl)pyridin-2-yl)-5-(((tert-butyldimethylsilyl)oxy)methyl)-5-methyl-2-(methylthio)-6,7-dihydro-5H-pyrrolo[2,3-d]pyrimidin-6-yl)acetate

A mixture of ethyl (E)-5-[tert-butyl(dimethyl)silyl]oxy-4-(4-chloro-2-methylsulfanyl-pyrimidin-5-yl)-4-methyl-pent-2-enoate (Intermediate 290, 250 mg, 579.96 umol, 1 eq.), tert-butyl N-[(6-amino-2-pyridyl)methyl]-N-methyl-carbamate (Intermediate 291, 206.43 mg, 869.94 umol, 1.5 eq.), Pd₂dba₃ (53.11 mg, 58.00 umol, 0.1 eq.), BINAP (72.23 mg, 115.99 umol, 0.2 eq.), Cs₂CO₃ (566.89 mg, 1.74 mmol, 3 eq.) in toluene (4 mL) was stirred at 100° C. for 16 h under nitrogen atmosphere. The reaction was allowed to cool down to room temperature, then was diluted with brine (50 mL) and was extracted with EA (3×50 mL). The combined organic layers were dried over anhydrous Na₂SO₄. After filtration, the filtrate was concentrated under reduced pressure. The residue was purified by silica gel column (eluting with 1:1 EA/PE) to afford ethyl 2-(7-(6-(((tert-butoxycarbonyl)(methyl)amino)methyl)pyridin-2-yl)-5-(((tert-butyldimethylsilyl)oxy)methyl)-5-methyl-2-(methylthio)-6,7-dihydro-5H-pyrrolo[2,3-d]pyrimidin-6-yl)acetate (210 mg, 0.33 mmol, 57% yield) as a yellow solid. LCMS (ES, m/z): 632 [M+H]⁺, Rt 1.244 min.

### Step 2. tert-butyl rac-((6-((5R,6R)-5-(((tert-butyldimethylsilyl)oxy)methyl)-6-(2-hydroxyethyl)-5-methyl-2-(methylthio)-5,6-dihydro-7H-pyrrolo[2,3-d]pyrimidin-7-yl)pyridin-2-yl)methyl)(methyl)carbamate

To a stirred solution of ethyl 2-(7-(6-(((tert-butoxycarbonyl)(methyl)amino)methyl)pyridin-2-yl)-5-(((tert-butyldimethylsilyl)oxy)methyl)-5-methyl-2-(methylthio)-6,7-dihydro-5H-pyrrolo[2,3-d]pyrimidin-6-yl)acetate (850 mg, 1.35 mmol) in THF (10 mL) was added LAH (2 M in THF, 1.35 mL, 2.69 mmol, 2 eq.) dropwise at −78° C. under the nitrogen atmosphere. The reaction was stirred for 1 h at −78° C. was then quenched with water/ice (100 mL). The resulting mixture was extracted with EA (3×100 mL). The combined organic layers were dried over anhydrous Na₂SO₄. After filtration, the filtrate was concentrated under reduced pressure. The residue was purified by silica gel column chromatography (eluting with 2:1 ethyl acetate/petroleum ether) to afford tert-butyl rac-((6-((5R,6R)-5-(((tert-butyldimethylsilyl)oxy)methyl)-6-(2-hydroxyethyl)-5-methyl-2-(methylthio)-5,6-dihydro-7H-pyrrolo[2,3-d]pyrimidin-7-yl)pyridin-2-yl)methyl)(methyl)carbamate (195 mg, 330.59 μmol, 25% yield). LCMS (ES, m/z): 590 [M+H]⁺, Rt 1.125 min.

### Step 3. rac-tert-butyl ((6-((5R,6R)-6-(2-hydroxyethyl)-5-(hydroxymethyl)-5-methyl-2-(methylthio)-5,6-dihydro-7H-pyrrolo[2,3-d]pyrimidin-7-yl)pyridin-2-yl)methyl)(methyl)carbamate

To a stirred solution of tert-butyl rac-((6-((5R,6R)-5-(((tert-butyldimethylsilyl)oxy)methyl)-6-(2-hydroxyethyl)-5-methyl-2-(methylthio)-5,6-dihydro-7H-pyrrolo[2,3-d]pyrimidin-7-yl)pyridin-2-yl)methyl)(methyl)carbamate (195 mg, 0.33 mmol, 1 eq.) in THF (8 mL) was added TBAF (1M in THF, 8 mL) dropwise at room temperature. The resulting mixture was stirred for 2 h at room temperature, then was diluted with water (50 mL) and extracted with EA (3×50 mL). The combined organic layers were washed with brine, dried over anhydrous Na₂SO₄. After filtration, the filtrate was concentrated under reduced pressure. The residue was purified by silica gel column chromatography (eluting with 1:1 EA/PE) to afford rac-tert-butyl ((6-((5R,6R)-6-(2-hydroxyethyl)-5-(hydroxymethyl)-5-methyl-2-(methylthio)-5,6-dihydro-7H-pyrrolo[2,3-d]pyrimidin-7-yl)pyridin-2-yl)methyl)(methyl)carbamate (130 mg, 0.27 mmol, 81% yield) as a yellow oil. ¹H NMR (400 MHz, CDCl3) δ 8.53 (d, J=8.4 Hz, 1H), 8.12 (s, 1H), 7.72 (t, J=7.6 Hz, 1H), 6.93-6.87 (m, 1H), 4.91-4.82 (m, 1H), 4.69-4.30 (m, 2H), 4.08-3.95 (m, 2H), 3.70-3.51 (m, 2H), 2.61 (s, 3H), 2.20-2.07 (m, 1H), 1.82-1.67 (m, 1H), 1.57-1.39 (m, 9H), 1.36 (s, 3H), 0.09 (s, 3H). LCMS (ES, m/z): 476 [M+H]⁺, Rt 0.760 min.

### Step 4. rac-tert-butyl methyl((6-((4bR,8aR)-4b-methyl-2-(methylthio)-4b,7,8,8a-tetrahydropyrano[3′,4′:4,5]pyrrolo[2,3-d]pyrimidin-9(5H)-yl)pyridin-2-yl)methyl)carbamate

To a stirred solution of rac-tert-butyl ((6-((5R,6R)-6-(2-hydroxyethyl)-5-(hydroxymethyl)-5-methyl-2-(methylthio)-5,6-dihydro-7H-pyrrolo[2,3-d]pyrimidin-7-yl)pyridin-2-yl)methyl)(methyl)carbamate (110 mg, 0.23 μmol, 1 eq.) and PPh₃ (121 mg, 0.46 mmol, 2 eq.) in THF (4 mL) was added DIAD (93 mg, 0.46 mmol, 2 eq.) dropwise at room temperature. The reaction was stirred for 3 h at 50° C. under the N₂ atmosphere and then was allowed to cool down to room temperature. The resulting mixture was concentrated under vacuum and the crude product was purified by reverse phase chromatography with the following conditions: (Column: C18; Mobile phase, A: water (containing 10 mmol/L NH₄HCO₃) and B: ACN (5% to 90% over 30 min); Detector, UV 254 nm) to afford rac-tert-butyl methyl((6-((4bR,8aR)-4b-methyl-2-(methylthio)-4b,7,8,8a-tetrahydropyrano[3′,4′:4,5]pyrrolo[2,3-d]pyrimidin-9(5H)-yl)pyridin-2-yl)methyl)carbamate (25 mg, 54.63 umol, 23% yield) as a yellow oil. LCMS (ES, m/z): 458 [M+H]⁺, Rt 0.914 min.

### Step 5-6. rac-tert-butyl methyl((6-((4bR,8aR)-4b-methyl-2-((4-morpholinophenyl)amino)-4b,7,8,8a-tetrahydropyrano[3′,4′:4,5]pyrrolo[2,3-d]pyrimidin-9(5H)-yl)pyridin-2-yl)methyl)carbamate

Following the experimental procedures described for Examples 1 and 2/step 5-6, rac-tert-butyl methyl((6-((4bR,8aR)-4b-methyl-2-((4-morpholinophenyl)amino)-4b,7,8,8a-tetrahydropyrano[3′,4′:4,5]pyrrolo[2,3-d]pyrimidin-9(5H)-yl)pyridin-2-yl)methyl)carbamate (18 mg, 27.85 umol, 42% yield) was obtained as a yellow oil, starting from rac-tert-butyl methyl((6-((4bR,8aR)-4b-methyl-2-(methylthio)-4b,7,8,8a-tetrahydropyrano[3′,4′:4,5]pyrrolo[2,3-d]pyrimidin-9(5H)-yl)pyridin-2-yl)methyl)carbamate (25 mg, 54.70 umol, 1 eq.). LCMS (ES, m/z): 588 [M+H]⁺, Rt 0.892 min.

### Step 7. rac-(4bR,8aR)-4b-methyl-9-(6-((methylamino)methyl)pyridin-2-yl)-N-(4-morpholinophenyl)-4b,5,7,8,8a,9-hexahydropyrano[3′,4′:4,5]pyrrolo[2,3-d]pyrimidin-2-amine

To a stirred solution of rac-tert-butyl methyl((6-((4bR,8aR)-4b-methyl-2-((4-morpholinophenyl)amino)-4b,7,8,8a-tetrahydropyrano[3′,4′:4,5]pyrrolo[2,3-d]pyrimidin-9(5H)-yl)pyridin-2-yl)methyl)carbamate (18 mg, 30.63 umol, 1 eq.) in DCM (2 mL) was added TFA (0.5 mL) dropwise. The resulting mixture was stirred for 1.5 h at room temperature and then was concentrated under vacuum. The crude product was purified by Prep-HPLC (Column: XBridge Shield RP18 OBD Column, 19*150 mm/5 μm; Mobile Phase A: Water (10 mmol/L NH₄HCO₃+0.1% NH₃·H₂O), Mobile Phase B: ACN; Flow rate: 25 mL/min; Gradient: 33% B to 53% B in 7 min, 53% B; Wave Length: 254 nm; RT1(min): 6.17) to afford rac-(4bR,8aR)-4b-methyl-9-(6-((methylamino)methyl)pyridin-2-yl)-N-(4-morpholinophenyl)-4b,5,7,8,8a,9-hexahydropyrano[3′,4′:4,5]pyrrolo[2,3-d]pyrimidin-2-amine (10 mg, 20.49 umol, 66% yield) as a white solid. ¹H NMR (300 MHz, DMSO-d6) δ 9.11 (s, 1H), 8.41 (d, J=8.1 Hz, 1H), 8.06 (s, 1H), 7.77 (t, J=8.1 Hz, 1H), 7.60 (d, J=8.1 Hz, 2H), 7.08 (d, J=7.2 Hz, 1H), 6.92 (d, J=8.4 Hz, 2H), 4.67-4.63 (m, 1H), 4.06-4.02 (m, 1H), 3.75-3.64 (m, 7H), 3.56-3.45 (m, 2H), 3.07-3.04 (m, 4H), 2.43-2.28 (m, 4H), 1.56-1.51 (m, 1H), 1.19 (s, 3H). LCMS (ES, m/z): 488 [M+H]⁺. Rt 0.527 min.

### Step 8. (4bR,8aR)-4b-methyl-9-(6-((methylamino)methyl)pyridin-2-yl)-N-(4-morpholinophenyl)-4b,5,7,8,8a,9-hexahydropyrano[3′,4′:4,5]pyrrolo[2,3-d]pyrimidin-2-amine and (4bS,8aS)-4b-methyl-9-(6-((methylamino)methyl)pyridin-2-yl)-N-(4-morpholinophenyl)-4b,5,7,8,8a,9-hexahydropyrano[3′,4′:4,5]pyrrolo[2,3-d]pyrimidin-2-amine

Rac-(4bR,8aR)-4b-methyl-9-(6-((methylamino)methyl)pyridin-2-yl)-N-(4-morpholinophenyl)-4b,5,7,8,8a,9-hexahydropyrano[3′,4′:4,5]pyrrolo[2,3-d]pyrimidin-2-amine (8 mg, 16.41 umol, 1 eq.) was separated by chiral-HPLC (Column: (R, R)-WHELK-O1-Kromasil, 5*25 cm/5 μm; Mobile Phase A: Hex:DCM=3:1 (0.5% 2M NH₃-MeOH), Mobile Phase B: EtOH; Flow rate: 20 mL/min; Gradient: 10% B in 14 min; Wave Length: 220/254 nm; RT1(min): 10.077; RT2(min): 12.969) to afford (4bR,8aR)-4b-methyl-9-(6-((methylamino)methyl)pyridin-2-yl)-N-(4-morpholinophenyl)-4b,5,7,8,8a,9-hexahydropyrano[3′,4′:4,5]pyrrolo[2,3-d]pyrimidin-2-amine (3.3 mg, 40% yield) and (4bS,8aS)-4b-methyl-9-(6-((methylamino)methyl)pyridin-2-yl)-N-(4-morpholinophenyl)-4b,5,7,8,8a,9-hexahydropyrano[3′,4′:4,5]pyrrolo[2,3-d]pyrimidin-2-amine (3.2 mg, 39% yield) as a white solids.

### Examples 572 and 573: ¹H NMR (300 MHz, DMSO-d6) δ 9.11 (s, 1H), 8.41 (d, J=8.1 Hz, 1H), 8.06 (s, 1H), 7.77 (t, J=8.1 Hz, 1H), 7.60 (d, J=8.1 Hz, 2H), 7.08 (d, J=7.5 Hz, 1H), 6.92 (d, J=8.7 Hz, 2H), 4.67-4.63 (m, 1H), 4.06-4.02 (m, 1H), 3.75-3.64 (m, 7H), 3.56-3.44 (m, 2H), 3.07-3.04 (m, 4H), 2.43-2.27 (m, 4H), 1.54-1.51 (m, 1H), 1.17 (s, 3H). LCMS (ES, m/z): 488 [M+H]⁺. Rt 0.525 min.

¹H NMR (300 MHz, DMSO-d6) δ 9.11 (s, 1H), 8.41 (d, J=8.4 Hz, 1H), 8.06 (s, 1H), 7.77 (t, J=8.1 Hz, 1H), 7.60 (d, J=8.4 Hz, 2H), 7.07 (d, J=7.2 Hz, 1H), 6.92 (d, J=8.7 Hz, 2H), 4.67-4.62 (m, 1H), 4.06-4.02 (m, 1H), 3.75-3.64 (m, 7H), 3.56-3.42 (m, 2H), 3.07-3.04 (m, 4H), 2.43-2.28 (m, 4H), 1.54-1.51 (m, 1H), 1.17 (s, 3H). LCMS (ES, m/z): 488 [M+H]⁺. Rt 0.525 min.

### Intermediate 300. (4bR,8aR)-4b,7,7-trimethyl-2-(methylthio)-4b,5,7,8,8a,9-hexahydropyrano[3′,4′:4,5]pyrrolo[2,3-d]pyrimidine

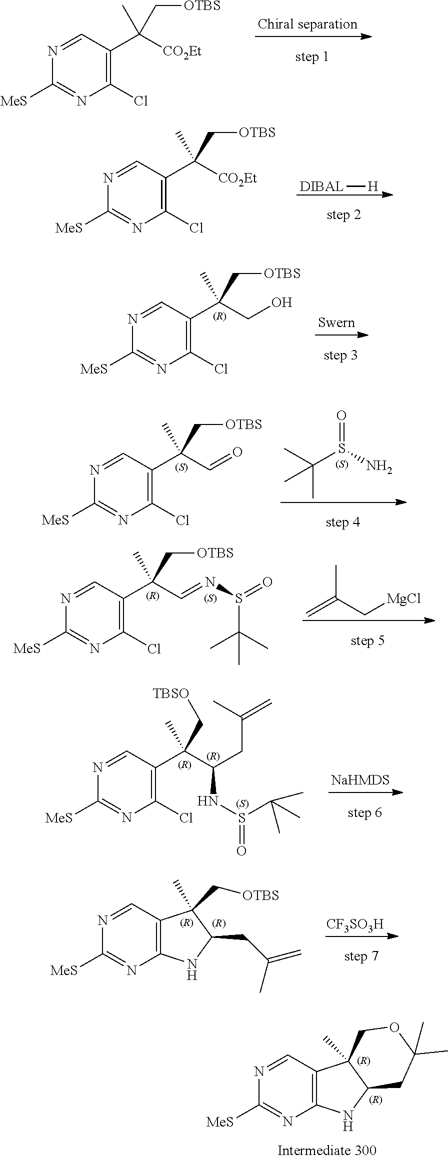

### Step 1. ethyl (2S)-3-[tert-butyl(dimethyl)silyl]oxy-2-(4-chloro-2-methylsulfanyl-pyrimidin-5-yl)-2-methyl propanoate and ethyl (2R)-3-[tert-butyl(dimethyl)silyl]oxy-2 (4 chloro-2-methylsulfanyl-pyrimidin-5-yl)-2-methyl-propanoate

The mixture of ethyl 3-[tert-butyl(dimethyl)silyl]oxy-2-(4-chloro-2-methylsulfanyl-pyrimidin-5-yl)-2-methyl-propanoate (Intermediate 1/step 4, 63 g, 155.55 mmol, 1 eq) was separated by Prep-CHIRAL-HPLC (CHIRAL ART Cellulose-SC, 7*25 cm, 10 rm; Mobile Phase A: CO₂, Mobile Phase B: IPA:HEX=1:1; Flow rate: 250 mL/min; Gradient: isocratic 20% B; Column Temperature(° C.): 35; Back Pressure(bar): 100; Wave Length: 220 nm; RT1(min): 6.78; RT2(min): 8.26) to afford ethyl (2S)-3-[tert-butyl(dimethyl)silyl]oxy-2-(4-chloro-2-methylsulfanyl-pyrimidin-5-yl)-2-methyl-propanoate (27 g, 66.00 mmol, 43% yield) (first eluting isomer) as a yellow oil and ethyl (2R)-3-[tert-butyl(dimethyl)silyl]oxy-2-(4-chloro-2-methylsulfanyl-pyrimidin-5-yl)-2-methyl-propanoate (24 g, 58.66 mmol, 38% yield) (second eluting isomer) as a yellow oil. LCMS (ES, m/z):405 [M+H]+, Rt 1.038 min.

### Step 2. (2R)-3-[tert-butyl(dimethyl)silyl]oxy-2-(4-chloro-2-methylsulfanyl-pyrimidin-5-yl)-2-methyl-propan-1-ol

A solution of ethyl (2S)-3-[tert-butyl(dimethyl)silyl]oxy-2-(4-chloro-2-methylsulfanyl-pyrimidin-5-yl)-2-methyl-propanoate (10.00 g, 24.68 mmol, 1 eq) in DCM (200 mL) was added DIBAL-H (1 M in DCM, 62 mL, 2.5 eq.) dropwise with stirring at −78° C. The mixture was stirred for 2.5 h at −78° C. The reaction was quenched with water (2.4 mL) at −78° C. The mixture was raised up to 0° C. and stirred at 0° C. for 10 min. Then NaOH (15%, 2.4 mL) was added. The mixture was stirred at 0-5° C. for 10 min. Then H₂O (6.0 mL) was added. The mixture was stirred at room temperature for 1 h. The reaction mixture was added Na₂SO₄ (30 g), stirred at room temperature for 0.5 h. Then filtered, the filtered cake was washed with DCM (50 mL×4). The organic layers were dried over sodium sulfate, filtered. The organic layer was concentrated under reduced pressure to afford (2R)-3-[tert-butyl(dimethyl)silyl]oxy-2-(4-chloro-2-methylsulfanyl-pyrimidin-5-yl)-2-methyl-propan-1-ol (8 g, crude), which was used in the next step directly without further purification. LCMS (ES, m/z): 363 [M+H]+, Rt 0.933 min.

### Step 3. (2S)-3-[tert-butyl(dimethyl)silyl]oxy-2-(4-chloro-2-methylsulfanyl-pyrimidin-5-yl)-2-methyl-propanal

Into a 3-necked flask was added DCM (100 mL) and oxalyl chloride (7 g, 55.10 mmol, 4.81 mL, 2 eq) at −50° C. Then DMSO (5.38 g, 68.87 mmol, 4.89 mL, 2.5 eq) was added dropwise at −50° C. The mixture was stirred at −50° C. for 0.5 h. To the above mixture, (2R)-3-[tert-butyl(dimethyl)silyl]oxy-2-(4-chloro-2-methylsulfanyl-pyrimidin-5-yl)-2-methyl-propan-1-ol (10 g, 27.55 mmol, 1 eq) was added dropwise at −50° C. After addition, this mixture was stirred at −50° C. for 1 h. Then TEA (11.15 g, 110.20 mmol, 15.36 mL) was added dropwise at −50° C. The mixture was stirred at −50° C. for 1 h. The reaction solution was quenched with water (100 mL). The resulting solution was extracted with DCM (300 mL×2). The organic layers were combined, washed with water (200 mL×3). The organic layers were dried over anhydrous sodium sulfate and concentrated to afford (2S)-3-[tert-butyl(dimethyl)silyl]oxy-2-(4-chloro-2-methylsulfanyl-pyrimidin-5-yl)-2-methyl-propanal (10 g, crude) as a yellow oil, which was used in the next step directly without further purification. LCMS (ES, m/z): 361 [M+H]⁺, Rt 0.947 min.

### Step 4. (S)—N—((R,E)-3-((tert-butyldimethylsilyl)oxy)-2-(4-chloro-2-(methylthio)pyrimidin-5-yl)-2-methylpropylidene)-2-methylpropane-2-sulfinamide

A solution of (2S)-3-[tert-butyl(dimethyl)silyl]oxy-2-(4-chloro-2-methylsulfanyl-pyrimidin-5-yl)-2-methyl-propanal (20 g, 55.41 mmol, 1 eq.) in THF (200 mL) was added (S)-2-methylpropane-2-sulfinamide (13.43 g, 110.81 mmol, 2 eq) and Ti(EtO)₄ (25.28 g, 110.81 mmol, 2 eq.). The mixture was stirred at room temperature for 16 h. Then the solution was poured into sat. Na₂CO₃ (50 mL). The mixture was stirred at room temperature for 2 h. The resulting solution was filtered, the filtered cake was washed with EA (50 mL×4). The filtrate was dried over anhydrous sodium sulfate and concentrated. The residue was purified by silica gel column chromatography (eluting with 3:1 PE/EA) to afford (S)—N—((R,E)-3-((tert-butyldimethylsilyl)oxy)-2-(4-chloro-2-(methylthio)pyrimidin-5-yl)-2-methylpropylidene)-2-methylpropane-2-sulfinamide (18.5 g, 37.86 mmol, 72% yield) as a yellow oil. LCMS (ES, m/z): 464 [M+H]+, Rt 1.400 min.

### Step 5. (S)—N-[(1R)-1-[(1R)-2-[tert-butyl(dimethyl)silyl]oxy-1-(4-chloro-2-methylsulfanyl-pyrimidin-5-yl)-1-methyl-ethyl]-3-methyl-but-3-enyl]-2-methyl-propane-2-sulfinamide

To a solution of (S)—N—((R,E)-3-((tert-butyldimethylsilyl)oxy)-2-(4-chloro-2-(methylthio)pyrimidin-5-yl)-2-methylpropylidene)-2-methylpropane-2-sulfonamide (8.5 g, 18.31 mmol, 1 eq) in DCM (170 mL) was added chloro(2-methylallyl)magnesium (0.5 M in THF, 38.5 mL, 1.05 eq) at −50° C. dropwise under N₂ atmosphere. The resulting mixture was stirred for 1 h at −50° C. under N₂ atmosphere. The reaction was quenched with ice/water. The resulting mixture was extracted with EA (3×100 mL). The combined organic layers were washed with brine (2×100 mL), dried over anhydrous Na₂SO₄. After filtration, the filtrate was concentrated under reduced pressure. The residue was purified by silica gel column chromatography (eluting with 2:1 PE/EA) to afford (S)—N-[(1R)-1-[(1R)-2-[tert-butyl(dimethyl) silyl]oxy-1-(4-chloro-2-methylsulfanyl-pyrimidin-5-yl)-1-methyl-ethyl]-3-methyl-but-3-enyl]-2-methyl-propane-2-sulfinamide (7.2 g, 13.15 mmol, 76% yield) as a yellow oil. LCMS (ES, m/z): 520 [M+H]⁺, Rt 0.958 min.

### Step 6. tert-butyl-dimethyl-[[(5R,6R)-5-methyl-6-(2-methylallyl)-2-methylsulfanyl-6,7-dihydropyrrolo[2,3-d]pyrimidin-5-yl]methoxy]silane

To a solution of (S)—N-[(1R)-1-[(1R)-2-[tert-butyl(dimethyl)silyl]oxy-1-(4-chloro-2-methylsulfanyl-pyrimidin-5-yl)-1-methyl-ethyl]-3-methyl-but-3-enyl]-2-methyl-propane-2-sulfinamide (4 g, 7.69 mmol, 1 eq.) in THF (40 mL) was added NaHMDS (2 M in THF, 11.53 mL, 3 eq) at −50° C. The resulting mixture was stirred for 3 h at room temperature under the N₂ atmosphere. The reaction was quenched with ice/water(100 mL). The resulting mixture was extracted with EA (3×200 mL). The combined organic layers were washed with brine (2×200 mL), dried over anhydrous Na₂SO₄. After filtration, the filtrate was concentrated under reduced pressure to afford tert-butyl-dimethyl-[[(5R,6R)-5-methyl-6-(2-methylallyl)-2-methylsulfanyl-6,7-dihydropyrrolo[2,3-d]pyrimidin-5-yl]methoxy]silane (1.9 g, crude), which was used in the next step directly without further purification. LCMS (ES, m/z): 380 [M+H]⁺, Rt 0.747 min.

### Step 7. (4bR,8aR)-4b,7,7-trimethyl-2-(methylthio)-4b,5,7,8,8a,9-hexahydropyrano[3′,4′:4,5]pyrrolo[2,3-d]pyrimidine (Intermediate 300)

To a solution of tert-butyl-dimethyl-[[(5R,6R)-5-methyl-6-(2-methylallyl)-2-methylsulfanyl-6,7-dihydropyrrolo[2,3-d]pyrimidin-5-yl]methoxy]silane (3.2 g, 8.43 mmol, 1 eq.) in DCM (10 mL) was added CF₃SO₃H (2.08 g, 25.29 mmol, 3 eq.) at 0° C. The resulting mixture was stirred for 2 h at room temperature. The reaction was monitored by LCMS. The mixture was basified to pH=8 with Na₂CO₃ (aq) at 0° C. The resulting mixture was extracted with DCM (3×100 mL). The combined organic layers were washed with brine (2×100 mL), dried over anhydrous Na₂SO₄. After filtration, the filtrate was concentrated under reduced pressure. The residue was purified by silica gel column chromatography (eluting with 1:1 PE/EA) to afford (4bR,8aR)-4b,7,7-trimethyl-2-(methylthio)-4b,5,7,8,8a,9-hexahydropyrano[3′,4′:4,5]pyrrolo[2,3-d]pyrimidine (1.7 g, 6.09 mmol, 76% yield) as a yellow solid. LCMS (ES, m/z): 266 [M+H]⁺, Rt 0.850 min. ¹H NMR (400 MHz, DMSO-d6) δ 7.77 (s, 1H), 7.66 (s, 1H), 3.77-3.51 (m, 2H), 3.48-3.41 (m, 1H), 2.37 (s, 3H), 1.77-1.68 (m, 1H), 1.59-1.48 (m, 1H), 1.21 (s, 3H), 1.14 (s, 6H).

### Intermediate 301. Dimethyl((6-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)imino)-l6-sulfanone

To a stirred mixture of (4bR,8aR)-4b,7,7-trimethyl-2-(methylthio)-4b,5,7,8,8a,9-hexahydropyrano[3′,4′:4,5]pyrrolo[2,3-d]pyrimidine (Intermediate 300, 500 mg, 1.88 mmol, 1 eq.) and (3-bromophenyl)imino-dimethyl-oxo-sulfane (Intermediate 53, 514.28 mg, 2.07 mmol, 1.2 eq.) in dioxane (10 mL) was added BINAP (245.21 mg, 376.83 μmol, 0.2 eq.), Pd₂(dba)₃ (172.53 mg, 188.41 μmol, 0.1 eq.) and dicesium carbonate (1.84 g, 5.65 mmol, 3 eq.) at room temperature. The resulting mixture was stirred for 2 h at 100° C. under N₂ atmosphere. The mixture was allowed to cool down to room temperature.

Desired product could be detected by LCMS. The solids were filtered out and washed with EA (3×10 mL). The filtrate was concentrated under reduced pressure. The residue was purified by silica gel column chromatography (eluting with 1:1 PE/EA) to afford dimethyl((6-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)imino)-l6-sulfanone (750 mg, 1.66 mmol, 88% yield) as a yellow solid. LCMS (ES, m/z): 434 [M+H]+, Rt 0.719 min.

### Intermediate 302 Dimethyl((6-((4bR,8aR)-4b,7,7-trimethyl-2-(methylsulfinyl)-4b,7,8,8a-tetra-hydropyrano[3′,4′:4,5]pyrrolo[2,3-d]pyrimidin-9(5H)-yl)pyridin-2-yl)imino)-l6-sulfanone

To a stirred mixture of dimethyl((6-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)pyridin-2-yl)imino)-l6-sulfanone (Intermediate 301, 400 mg, 0.92 mmol, 1.0 eq.) in DCM (15 mL) was added m-CPBA (191 mg, 1.11 mmol, 1.2 eq.) under 0° C. The resulting mixture was stirred for 3 h at room temperature. The reaction was monitored by LC-MS. The resulting mixture was concentrated under reduced pressure to afford dimethyl((6-((4bR,8aR)-4b,7,7-trimethyl-2-(methylsulfinyl)-4b,7,8,8a-tetrahydropyrano[3′,4′:4,5]pyrrolo[2,3-d]pyrimidin-9(5H)-yl)pyridin-2-yl)imino)-l6-sulfanone (400 mg, crude) as a yellow oil. LCMS (ES, m/z): 450 [M+H]⁺, Rt 0.897 min.

### Example 100: Dimethyl((6-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)pyridin-2-yl)imino)-l6-sulfanone

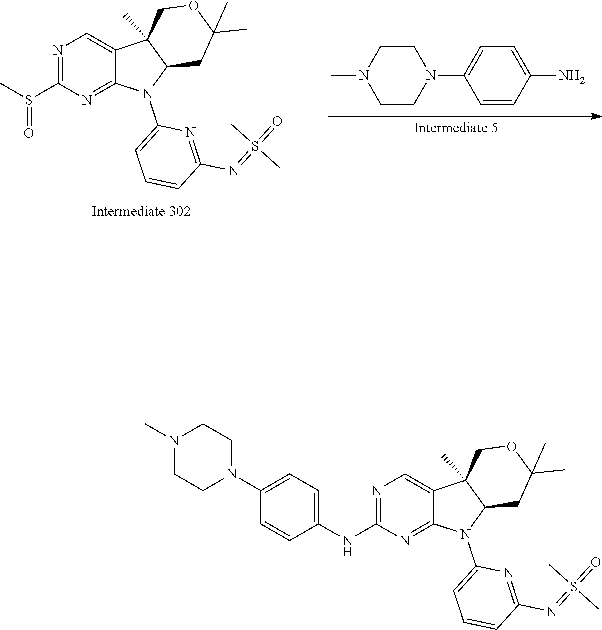

To a stirred mixture of dimethyl((6-((4bR,8aR)-4b,7,7-trimethyl-2-(methylsulfinyl)-4b,7,8,8a-tetrahydropyrano[3′,4′:4,5]pyrrolo[2,3-d]pyrimidin-9(5H)-yl)pyridin-2-yl)imino)-l6-sulfanone (Intermediate 302, 700 mg, 1.56 mmol, 1.0 eq.) in DIEA (3 mL) and toluene (3 mL) was added 4-(4-methyl piperazin-1-yl)aniline (Intermediate 5, 655 mg, 3.43 mmol, 2.2 eq.) at room temperature. The resulting mixture was stirred for 12 h at 110° C. The reaction was monitored by LC-MS. The resulting mixture was allowed to cool down to room temperature. The resulting mixture was concentrated under reduced pressure. The crude product was purified by reverse phase chromatography (C18 Column, 330 g; Mobile Phase A: Water (10 mmol/L NH₄HCO₃+0.1% NH₃·H₂O), Mobile Phase B: ACN (30% to 30% in 15 min); Flow rate: 90 mL/min;) to afford dimethyl((6-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)pyridin-2-yl)imino)-l6-sulfanone (555.7 mg, 0.95 mmol, 61% yield, 99% purity) as a light-brown solid.

¹H NMR (400 MHz, DMSO-d₆) δ 9.06 (s, 1H), 8.11 (d, J=8.0 Hz, 1H), 7.97 (s, 1H), 7.56-7.52 (m, 3H), 6.89-6.88 (m, 2H), 6.31 (d, J=8.0 Hz, 1H), 4.53-4.50 (m, 1H), 3.82 (d, J=12.4 Hz, 1H), 3.64 (d, J=12.4 Hz, 1H), 3.49 (s, 3H), 3.29 (s, 3H), 3.07-3.05 (m, 4H), 2.50-2.44 (m, 5H), 2.22 (s, 3H), 1.52-1.47 (m, 1H), 1.26 (s, 3H), 1.23 (s, 3H), 1.01 (s, 3H). LCMS (ES, m/z): 577 [M+H]⁺, Rt 0.561 min.

TABLE 18
The examples in the following table were synthesized according to the
appropriate method as demonstrated in Example 100.

| | Ex. | | |
|---|---|---|---|
| Structure | # | ¹H NMR | LCMS |
| 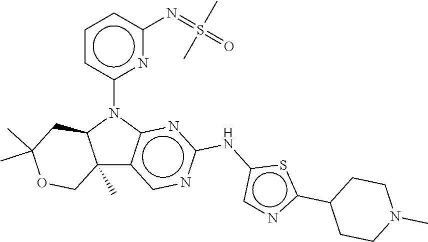 | 221 | 1H NMR (400 MHz, DMSO-d6) δ 10.33 (s, 1H), 8.09 (br, 1H), 8.04 (s, 1H), 7.55 (t, J = 8.0 Hz, 1H), 7.30 (s, 1H), 6.36 (d, J = 7.6 Hz, 1H), 4.57-4.53 (m, 1H), 3.84-3.81 (m, 1H), 3.66-3.63 (m, 1H), 3.48 (s, 3H), 2.30 (s, 3H), 2.83-2.76 (m, 3H), 2.46-2.41 (m, 1H), 2.18 (s, 3H), 2.01-1.95 (m, 4H), 1.73-1.63 (m, 2H), 1.55-1.49 (m, 1H), 1.27 (s, 3H), 1.22 (s, 3H), 1.00 (s, 3H). | LCMS (ES, m/z): 583 [M + H]+, Rt 1.023 min. |
| 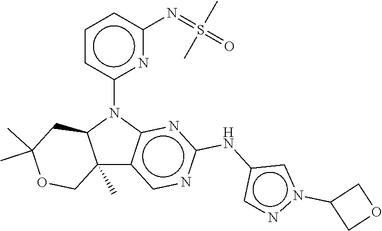 | 223 | 1H NMR (400 MHz, DMSO-d6) δ 9.19 (s, 1H), 8.05-7.98 (m, 3H), 7.60-7.54 (m, 2H), 6.32 (d, J = 7.8 Hz, 1H), 5.61-5.52 (m, 1H), 4.94-4.83 (m, 4H), 4.52-4.51 (m, 1H), 3.82-3.78 (m, 1H), 3.65-3.61 (m, 1H), 3.47 (s, 3H), 3.29 (s, 3H), 2.49-2.48 (m, 1H), 1.55-1.49 (m, 1H), 1.25 (s, 3H), 1.22 (s, 3H), 1.01 (s, 3H). | LCMS (ES, m/z): 525 [M + H]+, Rt 0.583 min. |
| 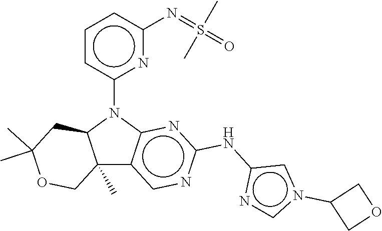 | 232 | 1H NMR (400 MHz, DMSO-d6) δ 9.31 (s, 1H), 8.13 (br, 1H), 8.02 (s, 1H), 7.60-7.55 (m, 3H), 6.32 (d, J = 7.6 Hz, 1H), 5.46-5.40 (m, 1H), 4.98 (t, J = 7.6 Hz, 2H), 4.78- 4.72 (m, 2H), 4.54-4.51 (m, 1H), 3.83-3.80 (m, 1H), 3.66-3.63 (m, 1H), 3.48 (s, 3H), 3.29 (s, 3H), 2.46-2.41 (m, 1H), 1.55-1.49 (m, 1H), 1.27 (s, 3H), 1.22 (s, 3H), 1.02 (s, 3H). | LCMS (ES, m/z): 525 [M + H]+, Rt 0.967 min. |
| 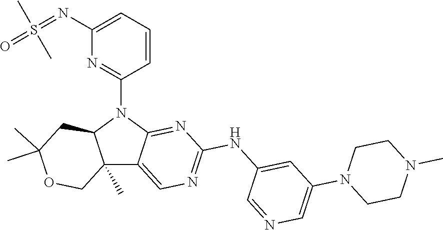 [SMILES: CN1CCN(c2cncc(Nc3ncc4c(n3)N(c3cccc(N=S(C)(C)=O)n3)[C@@H]3CC(C)(C)OC[C@]43C)c2)CC1] | 483 | 1H NMR (400 MHz, DMSO-d6) δ 9.36 (s, 1H), 8.31 (d, J = 2.0 Hz, 1H), 8.07 (s, 1H), 8.05 (d, J = 8.0 Hz, 1H), 7.91- 7.81 (m, 2H), 7.53 (t, J = 7.6 Hz, 1H), 6.34 (d, J = 7.6 Hz, 1H), 4.58-4.50 (m, 1H), 3.87-3.82 (m, 1H), 3.66-3.61 (m, 1H), 3.49 (s, 3H), 3.35 (s, 3H), 3.33-3.31 (m, 1H), 3.16-3.13 (m, 4H), 2.50-2.46 (m, 4H), 2.23 (s, 3H), 1.56-1.46 (m, 1H), 1.29 (s, 3H), 1.24 (s, 3H), 1.02 (s, 3H). | LCMS (ES, m/z): 578 [M + H]+; RT: 0.540 min. |
| 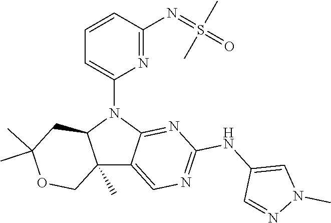 | 484 | 1H NMR (300 MHz, DMSO-d6) δ 9.11 (s, 1H), 8.02 (br, 1H), 7.97 (s, 1H), 7.83 (s, 1H), 7.56 (t, J = 7.8 Hz, 1H), 7.45 (s, 1H), 6.33 (d, J = 7.8 Hz, 1H), 4.53-4.48 (m, 1H), 3.83-3.79 (m, 4H), 3.65-3.62 (m, 1H), 3.48 (s, 3H), 3.33 (s, 3H), 2.50-2.41 (m, 1H), 1.54-1.46 (m, 1H), 1.26 (s, 3H), 1.22 (s, 3H), 1.01 (s, 3H). | LCMS (ES, m/z): 483 [M + H]+, Rt 0.578 min. |
| 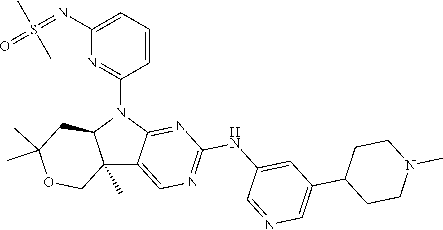 [SMILES: CN1CCC(c2cncc(Nc3ncc4c(n3)N(c3cccc(N=S(C)(C)=O)n3)[C@@H]3CC(C)(C)OC[C@]43C)c2)CC1] | 485 | 1H NMR (300 MHz, DMSO-d6) δ 9.47 (s, 1H), 8.67 (s, 1H), 8.15 (s, 1H), 8.08 (s, 1H), 8.05-7.97 (m, 2H), 7.57(d, J = 8.1 Hz, 1H), 6.33 (d, J = 7.8 Hz, 1H), 4.58- 4.53 (m, 1H), 3.87-3.82 (m, 1H), 3.66-3.61 (m, 1H), 3.49 (s, 3H), 3.30 (s, 3H), 2.88-2.82 (m, 2H), 2.49 (s, 3H), 2.20 (s, 3H), 1.95-1.91 (m, 2H), 1.76-1.61 (m, 3H), 1.56-1.52 (m, 1H), 1.27 (s, 3H), 1.24 (s, 3H), 1.02 (s, 3H). | LCMS (ES, m/z): 577 [M + H]+, Rt 0.869 min |
| 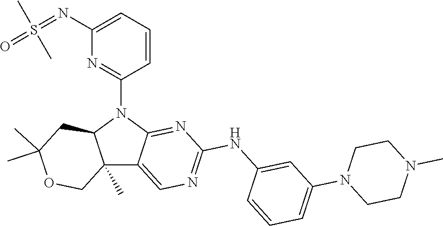 [SMILES: CN1CCN(c2cccc(Nc3ncc4c(n3)N(c3cccc(N=S(C)(C)=O)n3)[C@@H]3CC(C)(C)OC[C@]43C)c2)CC1] | 486 | 1H NMR (300 MHz, DMSO-d6) δ 9.14 (s, 1H), 8.11 (d, J = 8.1 Hz, 1H), 8.02 (s, 1H), 7.52 (t, J = 8.1 Hz, 1H), 7.37 (s, 1H), 7.19 (d, J = 8.1 Hz, 1H), 7.09 (t, J = 8.1 Hz, 1H), 6.52 (d, J = 8.4 Hz, 1H), 6.32 (d, J = 7.5 Hz, 1H), 4.55- 4.51 (m, 1H), 3.87-3.82 (m, 1H), 3.66-3.61 (m, 1H), 3.49 (s, 3H), 3.29 (s, 3H), 3.09-3.01 (m, 4H), 2.47-2.43 (m, 5H), 2.21 (s, 3H), 1.54-1.46 (m, 1H), 1.27 (s, 3H), 1.23 (s, 3H), 1.01 (s, 3H). | LCMS (ES, m/z): 577 [M + H]+, Rt 0.584 min. |
| 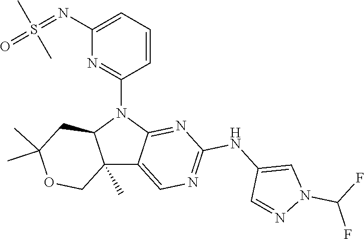 [SMILES: CC1(C)C[C@H]2N(c3cccc(N=S(C)(C)=O)n3)c3nc(Nc4cnn(C(F)F)c4)ncc3[C@@]2(C)CO1] | 487 | 1H NMR (400 MHz, DMSO-d6) δ 9.45 (s, 1H), 8.32 (s, 1H), 8.10-7.90 (m, 2H), 7.92-7.75 (m, 1H), 7.85-7.75 (m, 1H), 7.53 (d, J = 7.8 Hz, 1H), 6.32 (d, J = 8.0 Hz, 1H), 4.62-4.47 (m, 1H), 3.90-3.75 (m, 1H), 3.74-3.60 (m, 1H), 3.45 (s, 3H), 3.27 (s, 3H), 2.42-2.37 (m, 1H), 1.65- 1.45 (m, 1H), 1.27 (s, 3H), 1.22 (s, 3H), 1.02 (s, 3H). | LCMS (ES, m/z): 519 [M + H]+, Rt 0.663 min. |
| 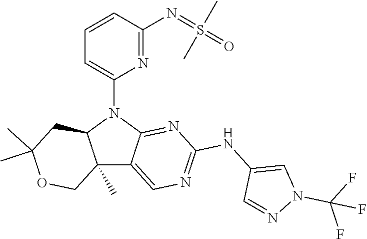 [SMILES: CC1(C)C[C@H]2N(c3cccc(N=S(C)(C)=O)n3)c3nc(Nc4cnn(C(F)(F)F)c4)ncc3[C@@]2(C)CO1] | 488 | 1H NMR (400 MHz, DMSO-d6) δ 9.58 (s, 1H), 8.43 (s, 1H), 8.06 (s, 1H), 7.95 (s, 1H), 7.54 (t, J = 8.0 Hz, 1H), 6.35 (d, J = 7.6 Hz, 1H), 4.62-4.50 (m, 1H), 3.87-3.80 (m, 1H), 3.72-3.57 (m, 1H), 3.48 (s, 3H), 3.25 (s, 3H), 2.47-2.46 (m, 1H), 2.40-2.35 (m, 1H), 1.61-1.52 (m, 1H), 1.27 (s, 3H), 1.22 (s, 3H), 1.01 (s, 3H). | LCMS (ES, m/z): 537 [M + H]+, Rt 0.731 min. |
| 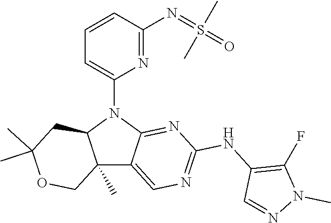 | 490 | 1H NMR (400 MHz, DMSO-d6) δ 8.50 (s, 1H), 7.92 (br, 1H), 7.90 (s, 1H), 7.49-7.42 (m, 2H), 6.29 (d, J = 7.6 Hz, 1H), 4.52-4.48 (m, 1H), 3.81-3.78 (m, 1H), 3.72 (s, 3H), 3.64-3.61 (m, 1H), 3.48 (s, 3H), 3.29 (s, 3H), 2.46-2.42 (m, 1H), 1.51-1.45 (m, 1H), 1.24 (s, 3H), 1.22 (s, 3H), 1.01 (s, 3H). | LCMS (ES, m/z): 501 [M + H]+, Rt 0.628 min. |
| 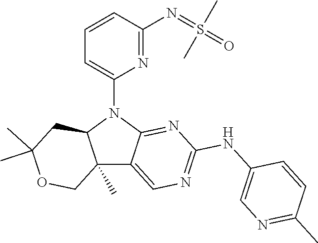 | 491 | 1H NMR (400 MHz, DMSO-d6) δ 9.38 (s, 1H), 8.73 (s, 1H), 8.11-8.03 (m, 3H), 7.55 (t, J = 8.0 Hz, 1H), 7.17 (d, J = 8.4 Hz, 1H), 6.34 (d, J = 8.0 Hz, 1H), 4.56-4.52 (m, 1H), 3.85-3.81 (m, 1H), 3.67-3.64 (m, 1H), 3.49 (s, 3H), 3.29 (s, 3H), 2.47-2.40 (m, 4H), 1.54-1.49 (m, 1H), 1.27 (s, 3H), 1.23 (s, 3H), 1.01 (s, 3H). | LCMS (ES, m/z): 494 [M + H]+, Rt 1.008 min. |
| 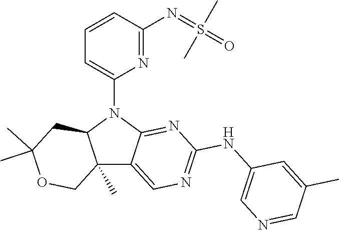 | 492 | 1H NMR (400 MHz, DMSO-d6) δ 9.48 (s, 1H), 8.61 (s, 1H), 8.14 (s, 1H), 7.07-7.98 (m, 3H), 7.55 (t, J = 8.0 Hz, 1H), 6.35 (d, J = 7.6 Hz, 1H), 4.57-4.53 (m, 1H), 3.85- 3.82 (m, 1H), 3.67-3.63 (m, 1H), 3.49 (s, 3H), 3.29 (s, 3H), 2.47-2.42 (m, 1H), 2.28 (s, 3H), 1.56-1.49 (m, 1H), 1.28 (s, 3H), 1.22 (s, 3H), 1.01 (s, 3H). | LCMS (ES, m/z): 494 [M + H]+, Rt 0.542 min. |
| 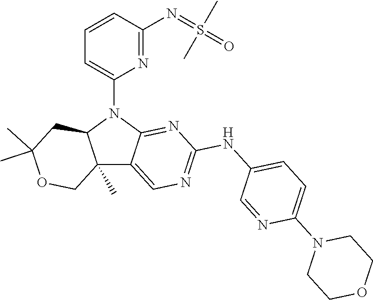 [SMILES: CC1(C)C[C@H]2N(c3cccc(N=S(C)(C)=O)n3)c3nc(Nc4ccc(N5CCOCC5)nc4)ncc3[C@@]2(C)CO1] | 493 | 1H NMR (400 MHz, DMSO-d6) δ 9.08 (s, 1H), 8.40 (s, 1H), 8.07 (d, J = 7.2 Hz, 1H), 7.94-7.93 (m, 2H), 7.52 (t, J = 8.0 Hz, 1H), 6.84 (d, J = 8.8 Hz, 1H), 6.32 (d, J = 7.6 Hz, 1H), 4.54-4.50 (m, 1H), 3.83-3.62 (m, 6H), 3.48 (s, 3H), 3.37-3.34 (m, 4H), 3.29 (s, 3H), 2.47-2.42 (m, 1H), 1.52-1.47 (m, 1H), 1.26 (s, 3H), 1.22 (s, 3H), 1.01 (s, 3H). | LCMS (ES, m/z): 565 [M + H]+, Rt 1.153 min. |
| 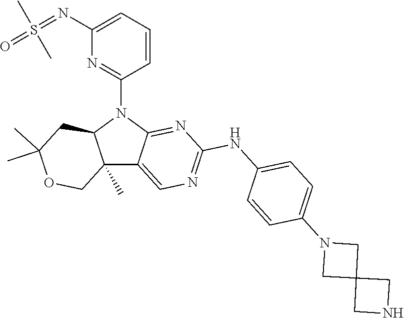 [SMILES: CC1(C)C[C@H]2N(c3cccc(N=S(C)(C)=O)n3)c3nc(Nc4ccc(N5CC6(CNC6)C5)cc4)ncc3[C@@]2(C)CO1] | 494 | 1H NMR (400 MHz, DMSO-d6) δ 8.94 (s, 1H), 8.09 (d, J = 8.0 Hz, 1H), 7.94 (s, 1H), 7.69-7.35 (m, 3H), 6.60-6.19 (m, 3H), 4.65-4.35 (m, 1H), 4.01-3.75 (m, 6H), 3.75- 3.59 (m, 4H), 3.58-3.42 (m, 4H), 3.31 (s, 3H), 2.49-2.41 (m, 1H), 1.65-1.45 (m, 1H), 1.28 (s, 3H), 1.23 (s, 3H), 1.01 (s, 3H). | LCMS (ES, m/z): 575 [M + H]+, Rt 0.569 min. |
| 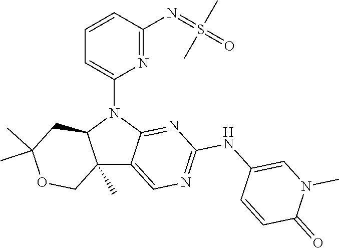 | 495 | 1H NMR (300 MHz, DMSO-d6) δ 8.92 (s, 1H), 8.10 (d, J = 2.1 Hz, 1H), 8.02-7.83 (m, 2H), 7.57-7.50 (m, 2H), 6.41-6.30 (m, 2H), 4.54-4.49 (m, 1H), 3.82-3.78 (m, 1H), 3.65-3.61 (m, 1H), 3.48 (s, 3H), 3.42 (s, 3H), 3.29 (s, 3H), 2.49-2.40 (m, 1H), 1.53-1.45 (m, 1H), 1.25 (s, 3H), 1.21 (s, 3H), 1.01 (s, 3H). | LCMS (ES, m/z): 510 [M + H]+, Rt 0.548 min. |
| 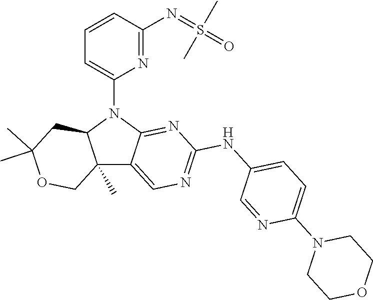 [SMILES: CC1(C)C[C@H]2N(c3cccc(N=S(C)(C)=O)n3)c3nc(Nc4ccc(N5CCOCC5)nc4)ncc3[C@@]2(C)CO1] | 496 | 1H NMR (400 MHz, DMSO-d6) δ 9.07 (s, 1H), 8.10 (d, J = 8.0 Hz, 1H), 7.97 (s, 1H), 7.62-7.50 (m, 3H), 6.89 (d, J = 9.2 Hz, 2H), 6.31 (d, J = 7.6 Hz, 1H), 4.56-4.51(m, 1H), 3.8-3.69 (m, 5H), 3.66-3.61 (m, 1H), 3.48 (s, 3H), 3.29 (s, 3H), 3.11-2.98 (m, 4H), 2.50-2.45 (m, 1H), 1.55-1.42 (m, 1H), 1.27 (s, 3H), 1.24 (s, 3H), 1.01 (s, 3H). | LCMS (ES, m/z): 564 [M + H]+, Rt 1.203 min. |
| 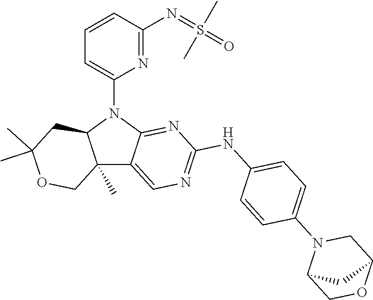 [SMILES: CC1(C)C[C@H]2N(c3cccc(N=S(C)(C)=O)n3)c3nc(Nc4ccc(N5C[C@@H]6C[C@H]5CO6)cc4)ncc3[C@@]2(C)CO1] | 497 | 1H NMR (400 MHz, DMSO-d6) δ 8.92 (s, 1H), 8.11 (d, J = 8.0 Hz, 1H), 7.94 (s, 1H), 7.69-7.22 (m, 3H), 6.57 (d, J = 8.8 Hz, 2H), 6.30 (d, J = 8.0 Hz, 1H), 4.61-4.43 (m, 3H), 3.86-3.61 (m, 4H), 3.48 (s, 4H), 3.29 (s, 3H), 2.92 (d, J = 9.2 Hz, 1H), 2.50-2.41 (m, 1H), 1.95-1.89 (m, 1H), 1.86-1.81 (m, 1H), 1.53-1.42 (m, 1H), 1.27 (s, 3H), 1.24 (s, 3H), 1.01 (s, 3H). | LCMS (ES, m/z): 576 [M + H]+, Rt 0.690 min. |
| 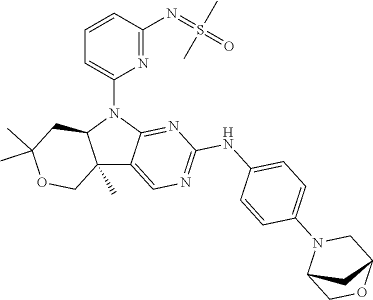 [SMILES: CC1(C)C[C@H]2N(c3cccc(N=S(C)(C)=O)n3)c3nc(Nc4ccc(N5C[C@H]6C[C@@H]5CO6)cc4)ncc3[C@@]2(C)CO1] | 498 | 1H NMR (400 MHz, DMSO-d6) δ 8.92 (s, 1H), 8.10 (d, J = 8.0 Hz, 1H), 7.94 (s, 1H), 7.65-7.35 (m, 3H), 6.58 (d, J = 8.8 Hz, 2H), 6.70-6.45 (m, 1H), 6.29 (d, J = 7.6 Hz, 1H), 4.71-4.55 (m, 1H), 4.54-4.38 (m, 2H), 3.95-3.79 (m, 1H), 3.79-3.60 (m, 2H), 3.60-3.40 (m, 4H), 3.28 (s, 3H), 3.05-2.80 (m, 1H), 2.48-2.41 (m, 1H), 2.05-1.90 (m, 1H), 1.90-1.74 (m, 1H), 1.62-1.38 (m, 1H), 1.28 (s, 3H), 1.23 (s, 3H), 1.01 (s, 3H). | LCMS (ES, m/z): 576 [M + H]+, Rt 0.682 min. |
| 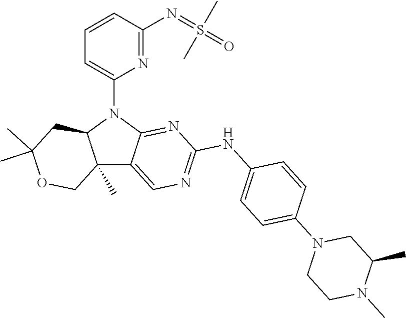 | 499 | 1H NMR (400 MHz, DMSO-d6) δ 8.98 (s, 1H), 8.04 (d, J = 8.0 Hz, 1H), 7.90 (s, 1H), 7.52-7.43 (m, 3H), 6.87 (d, J = 9.2 Hz, 2H), 6.24 (d, J = 7.8 Hz, 1H), 4.48-4.40 (m, 1H), 3.77-3.72 (m, 1H), 3.62-3.57 (m, 1H), 3.44-3.32 (m, 5H), 3.22 (s, 3H), 2.82-2.75 (m, 1H), 2.66-2.55 (m, 1H), 2.42-2.33 (m, 1H), 2.29-2.03 (m, 6H), 1.47-1.37 (m, 1H), 1.19 (s, 3H), 1.15 (s, 3H), 1.05 (d, J = 6.0 Hz, 3H), 1.01 (s, 3H). | LCMS (ES, m/z): 591 [M + H]+; RT: 0.554 min. |
| 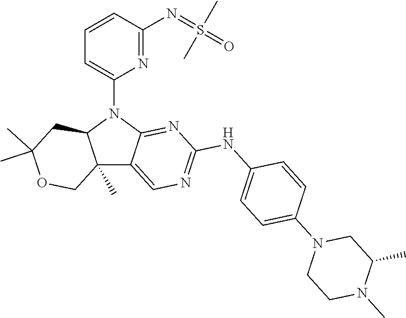 | 500 | 1H NMR (400 MHz, DMSO-d6) δ 8.98 (s, 1H), 8.04 (d, J = 8.0 Hz, 1H), 7.90 (s, 1H), 7.52-7.43 (m, 3H), 6.80 (d, J = 9.2 Hz, 2H), 6.24 (d, J = 7.8 Hz, 1H), 4.48-4.40 (m, 1H), 3.78-3.73 (m, 1H), 3.63-3.53 (m, 1H), 3.42 (s, 3H), 3.39-3.31 (m, 2H), 3.22 (s, 3H), 2.77-2.68 (m, 1H), 2.66-2.55 (m, 1H), 2.42-2.33 (m, 1H), 2.29-2.17 (m, 2H), 2.14 (s, 3H), 2.09-2.00 (m, 1H), 1.47-1.37 (m, 1H), 1.16 (s, 3H), 1.11 (s, 3H), 0.97 (d, J = 6.4 Hz, 3H), 0.95 (s, 3H). | LCMS (ES, m/z): 591 [M + H]+; RT: 0.566 min. |
| 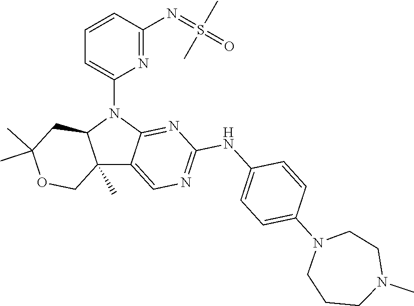 | 501 | 1H NMR (300 MHz, DMSO-d6) δ 8.87 (s, 1H), 8.11 (d, J = 8.1 Hz, 1H), 7.93 (s, 1H), 7.53-7.41 (m, 3H), 6.65-6.62 (m, 2H), 6.29 (d, J = 7.8 Hz, 1H), 4.52-4.47 (m, 1H), 3.82-3.78 (m, 1H), 3.65-3.61 (m, 1H), 3.57-3.47 (m, 5H), 3.43 (t, J = 6.3 Hz, 2H), 3.32 (s, 3H), 2.61-2.51 (m, 2H), 2.49-2.42 (m, 3H), 2.25 (s, 3H), 1.92-1.84 (m, 2H), 1.52-1.44 (m, 1H), 1.25 (s, 3H), 1.22 (s, 3H), 1.01 (s, 3H). | LCMS (ES, m/z): 591 [M + H]+, Rt 0.578 min. |
| 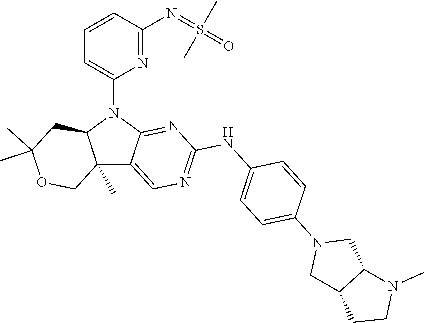 | 502 | 1H NMR (400 MHz, DMSO-d6) δ 8.91 (s, 1H), 8.11 (d, J = 8.0 Hz, 1H), 7.94 (s, 1H), 7.58-7.42 (m, 3H), 6.55 (d, J = 8.8 Hz, 2H), 6.30 (d, J = 7.6 Hz, 1H), 4.65-4.39 (m, 1H), 3.95-3.75 (m, 1H), 3.69-3.59 (m, 1H), 3.52 (s, 3H), 3.32-3.30 (m, 4H), 3.25-3.18 ( m, 1H), 3.17-3.01 (m, 2H), 3.00-2.92 (m, 1H), 2.90-2.81 (m, 2H), 2.48-2.42 (m, 1H), 2.32-2.29 (m, 3H), 2.25-2.18 (m, 1H), 2.18- 1.92 (m, 1H), 1.70-1.49 (m, 2H), 1.28 (s, 3H), 1.23 (s, 3H), 1.01 (s, 3H). | LCMS (ES, m/z): 603 [M + H]+, Rt 0.581 min. |
| 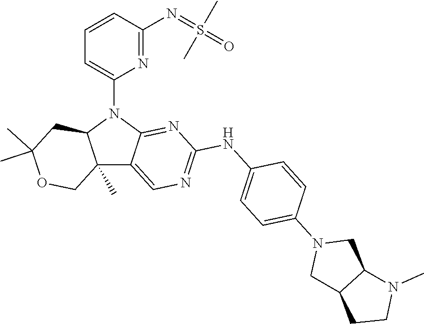 | 503 | 1H NMR (400 MHz, DMSO-d6) δ 8.91 (s, 1H), 8.11 (d, J = 8.0 Hz, 1H), 7.94 (s, 1H), 7.54-7.46 (m, 3H), 6.56 (d, J = 8.8 Hz, 2H), 6.30 (d, J = 7.6 Hz, 1H), 4.52-4.45 (m, 1H), 3.87-3.82 (m, 1H), 3.66-3.61 (m, 1H), 3.48 (s, 3H), 3.33-3.28 (m, 4H), 3.27-3.19 (m, 1H), 3.11-3.02 (m, 2H), 2.99-2.78 (m, 3H), 2.50-2.42 (m, 1H), 2.28 (s, 3H), 2.27-2.19 (m, 1H), 2.08-2.01 (m, 1H), 1.68-1.42 (m, 2H), 1.27 (s, 3H), 1.24 (s, 3H), 1.01 (s, 3H). | LCMS (ES, m/z): 603 [M + H]+, Rt 0.585 min. |
| 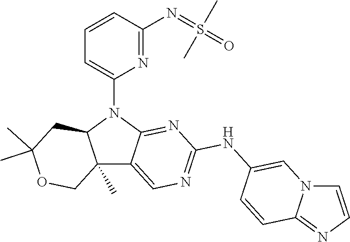 [SMILES: CC1(C)C[C@H]2N(c3cccc(N=S(C)(C)=O)n3)c3nc(Nc4ccc5nccn5c4)ncc3[C@@]2(C)CO1] | 504 | 1H NMR (400 MHz, DMSO-d6) δ 9.37 (s, 1H), 9.25 (s, 1H), 8.06 (d, J = 6.4 Hz, 2H), 7.88 (s, 1H), 7.61-7.47 (m, 3H), 7.41-7.34 (m, 1H), 6.35 (d, J = 7.86 Hz, 1H), 4.59- 4.51 (m, 1H), 3.87-3.82 (m, 1H), 3.66-3.61 (m, 1H), 3.49 (s, 3H), 3.30 (s, 3H), 2.49-2.40 (m, 1H), 1.58-1.48 (m, 1H), 1.29 (s, 3H), 1.23 (s, 3H), 1.02 (s, 3H). | LCMS (ES, m/z): 519 [M + H]+; RT: 1.063 min. |
| 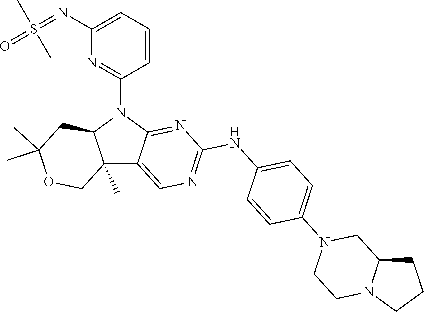 [SMILES: CC1(C)C[C@H]2N(c3cccc(N=S(C)(C)=O)n3)c3nc(Nc4ccc(N5CCN6CCC[C@@H]6C5)cc4)ncc3[C@@]2(C)CO1] | 505 | 1H NMR (300 MHz, DMSO-d6) δ 9.05 (s, 1H), 8.10 (d, J = 8.4 Hz, 1H), 7.96 (s, 1H), 7.57-7.52 (m, 3H), 6.89 (d, J = 9.0 Hz, 2H), 6.30 (d, J = 7.5 Hz, 1H), 4.52-4.49 (m, 1H), 3.83-3.79 (m, 1H), 3.69-3.61 (m, 2H), 3.55-3.48 (m, 4H), 3.29 (s, 3H), 3.05-3.01 (m, 2H), 2.68-2.66 (m, 1H), 2.94-2.43 (m, 1H), 2.37-2.30 (m, 1H), 2.23-2.22 (m, 1H), 2.07-2.01 (m, 2H), 1.72-1.67 (m, 3H), 1.55- 1.43 (m, 1H), 1.42-1.30 (m, 1H), 1.25 (s, 3H), 1.22 (s, 3H), 1.01 (s, 3H). | LCMS (ES, m/z): 603 [M + H]+, Rt 0.562 min. |
| 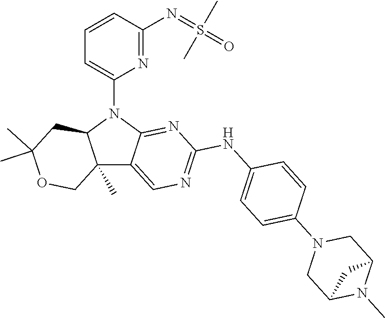 | 506 | 1H NMR (400 MHz, DMSO-d6) δ 8.94 (s, 1H), 8.14 (d, J = 8.0 Hz, 1H), 7.95 (s, 1H), 7.57-7.49 (m, 3H), 6.68 (d, J = 8.8 Hz, 2H), 6.31 (d, J = 7.6 Hz, 1H), 4.55-4.47 (m, 1H), 3.87-3.82 (m, 1H), 3.66-3.61 (m, 1H), 3.59 (d, J = 5.6 Hz, 2H), 3.53-3.38 (m, 6H), 3.35 (s, 2H), 3.27-3.22 (m, 2H), 2.50-2.39 (m, 2H), 2.00 (s, 3H), 1.58-1.45 (m, 2H), 1.27 (s, 3H), 1.24 (s, 3H), 1.02 (s, 3H). | LCMS (ES, m/z): 589 [M + H]+; RT: 1.122 min. |
| 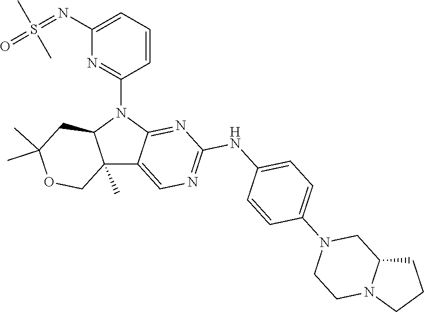 [SMILES: CC1(C)C[C@H]2N(c3cccc(N=S(C)(C)=O)n3)c3nc(Nc4ccc(N5CCN6CCC[C@H]6C5)cc4)ncc3[C@@]2(C)CO1] | 507 | 1H NMR (400 MHz, DMSO-d6) δ 9.04 (s, 1H), 8.10 (d, J = 8.0 Hz, 1H), 7.97 (s, 1H), 7.65-7.50 (m, 3H), 6.89 (d, J = 8.8 Hz, 2H), 6.30 (d, J = 8.0 Hz, 1H), 4.60-4.41 (m, 1H), 3.86-3.75 (m, 1H), 3.75-3.61 (m, 2H), 3.61-3.42 (m, 4H), 3.31 (s, 3H), 3.08-2.98 (m, 2H), 2.75-2.62 (m, 1H), 2.47-2.41 (m, 1H), 2.38-2.31 (m, 1H), 2.28-2.21 (m, 1H), 2.15-2.00 (m, 2H), 1.85-1.78 (m, 1H), 1.78- 1.66 (m, 2H), 1.60-1.50 (m, 1H), 1.45-1.32 (m, 1H), 1.28 (s, 3H), 1.22 (s, 3H), 1.01 (s, 3H). | LCMS (ES, m/z): 603 [M + H]+, Rt 0.568 min. |
| 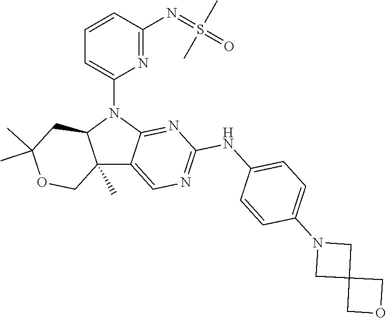 [SMILES: CC1(C)C[C@H]2N(c3cccc(N=S(C)(C)=O)n3)c3nc(Nc4ccc(N5CC6(COC6)C5)cc4)ncc3[C@@]2(C)CO1] | 508 | 1H NMR (400 MHz, DMSO-d6) δ 8.95 (s, 1H), 8.09 (d, J = 7.6 Hz, 1H), 7.94 (s, 1H), 7.53-7.47 (m, 3H), 6.40 (d, J = 8.8 Hz, 2H), 6.30 (d, J = 7.6 Hz, 1H), 4.71 (s, 4H), 4.52-4.48 (m, 1H), 3.94 (s, 4H), 3.82- 3.79 (m, 1H), 3.64-3.61 (m, 1H), 3.48 (s, 3H), 3.28 (s, 3H), 2.49-2.41 (m, 1H), 1.51-1.45 (m, 1H), 1.24 (s, 3H), 1.22 (s, 3H), 1.01 (s, 3H). | LCMS (ES, m/z): 576.25 [M + H]+, Rt 0.650 min. |
| 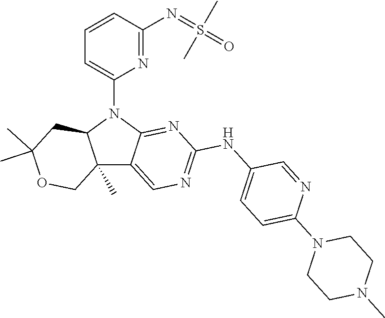 | 509 | 1H NMR (300 MHz, DMSO-d6) δ 9.06 (s, 1H), 8.36 (s, 1H), 8.06 (d, J = 8.1 Hz, 1H), 7.99-7.78 (m, 2H), 7.52 (t, J = 8.1 Hz, 1H), 6.82 (d, J = 9.0 Hz, 1H), 6.31 (d, J = 7.5 Hz, 1H), 4.65-4.38 (m, 1H), 3.82- 3.75 (m, 1H), 3.75-3.60 (m, 1H), 3.48 (s, 3H), 3.41- 3.35 (m, 4H), 3.29 (s, 3H), 2.47-2.35 (m, 5H), 2.27 (s, 3H), 1.62-1.35 (m, 1H), 1.25 (s, 3H), 1.22 (s, 3H), 1.01 (s, 3H). | LCMS (ES, m/z): 578.35 [M + H]+, Rt 0.618 min. |
| 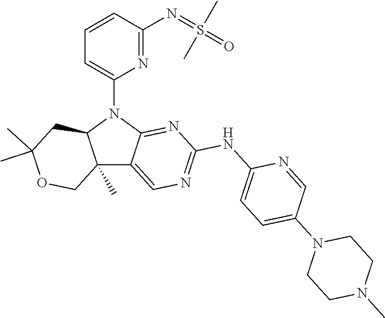 | 510 | 1H NMR (300 MHz, DMSO-d6) δ 9.24 (s, 1H), 8.17 (d, J = 8.1 Hz, 1H), 8.11-7.87 (m, 3H), 7.56 (t, J = 8.1 Hz, 1H), 7.47-7.37 (m, 1H), 6.32 (d, J = 7.8 Hz, 1H), 4.59-4.49 (m, 1H), 3.89-3.79 (m, 1H), 3.71- 3.60 (m, 2H), 3.49 (s, 3H), 3.29 (s, 3H), 3.16-3.07 (m, 4H), 2.52-2.39 (m, 4H), 2.23 (s, 3H), 1.58-1.45 (m, 1H), 1.27 (s, 3H), 1.23 (s, 3H), 1.02 (s, 3H). | LCMS (ES, m/z): 578.35 [M + H]+, Rt 0.572 min. |
| 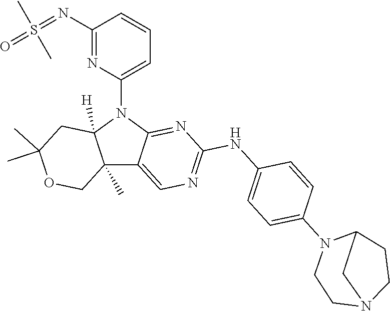 [SMILES: CC1(C)C[C@H]2N(c3cccc(N=S(C)(C)=O)n3)c3nc(Nc4ccc(N5CCN6CCC5C6)cc4)ncc3[C@@]2(C)CO1] | 511 | 1H NMR (300 MHz, DMSO-d6) δ 9.03 (s, 1H), 8.11 (d, J = 8.1 Hz, 1H), 7.96 (s, 1H), 7.58-7.47 (m, 3H), 6.87-6.79 (m, 2H), 6.30 (d, J = 7.8 Hz, 1H), 4.56- 4.46 (m, 1H), 4.21-4.12 (m, 1H), 3.89-3.77 (m, 1H), 3.71-3.60 (m, 1H), 3.48 (s, 3H), 3.35-3.31 (m, 1H), 3.29 (s, 3H), 3.05-2.89 (m, 3H), 2.85-2.60 (m, 4H), 2.47-2.39 (m, 1H), 1.71-1.56 (m, 2H), 1.54- 1.43 (m, 1H), 1.25 (s, 3H), 1.22 (s, 3H), 1.01 (s, 3H). | LCMS (ES, m/z): 589.35 [M + H]+, Rt 0.575 min. |
| 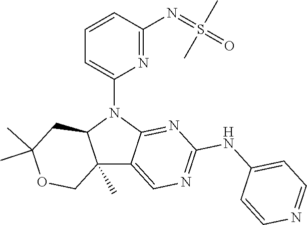 [SMILES: CC1(C)C[C@H]2N(c3cccc(N=S(C)(C)=O)n3)c3nc(Nc4ccncc4)ncc3[C@@]2(C)CO1] | 512 | 1H NMR (300 MHz, DMSO-d6) δ 8.66 (s, 2H), 8.35 (s, 1H), 7.83 (d, J = 8.0 Hz, 1H), 7.69 (t, J = 8.0 Hz, 1H), 6.64 (s, 2H), 6.63 (d, J = 8.0 Hz, 1H), 4.76- 4.62 (m, 1H), 3.95-3.86 (m, 1H), 3.75-3.68 (m, 1H), 3.49 (s, 3H), 3.31 (s, 3H), 2.48-2.40 (m, 1H), 1.70-1.55 (m, 1H), 1.31 (s, 3H), 1.21 (s, 3H), 1.00 (s, 3H). | LCMS (ES, m/z): 480.20 [M + H]+, Rt 0.633 min. |
| 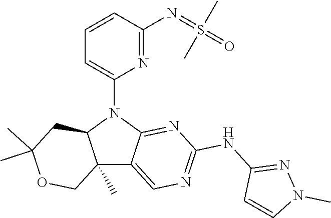 | 513 | 1H NMR (300 MHz, DMSO-d6) δ 9.33 (s, 1H), 8.16 (d, J = 8.1 Hz, 1H), 7.96 (s, 1H), 7.58-7.43 (m, 2H), 6.49 (d, J = 1.8 Hz, 1H), 6.30 (d, J = 7.8 Hz, 1H), 4.58-4.44 (m, 1H), 3.87-3.79 (m, 1H), 3.73 (s, 3H), 3.69-3.61 (m, 1H), 3.47 (s, 3H), 3.28 (s, 3H), 2.48- 2.37 (m, 1H), 1.58-1.41 (m, 1H), 1.25 (s, 3H), 1.22 (s, 3H), 1.01 (s, 3H). | LCMS (ES, m/z): 483.25 [M + H]+, Rt 1.233 min. |
| 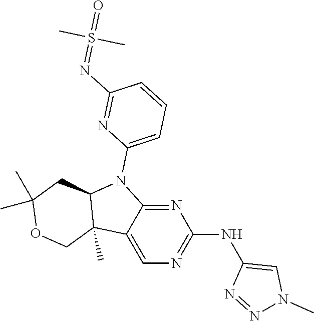 | 514 | 1H NMR (400 MHz, DMSO-d6) δ 9.86 (s, 1H), 8.21-7.95 (m, 3H), 7.57 (t, J = 8.0 Hz, 1H), 6.33 (d, J = 8.0 Hz, 1H), 4.60-4.43 (m, 1H), 4.03 (s, 3H), 3.89-3.79 (m, 1H), 3.68-3.61 (m, 1H), 3.48 (s, 3H), 3.29 (s, 3H), 2.46-2.40 (m, 1H), 1.55-1.47 (m, 1H), 1.27 (s, 3H), 1.22 (s, 3H), 1.01 (s, 3H). | LCMS (ES, m/z): 484.10 [M + H]+, Rt 0.858 min. |
| 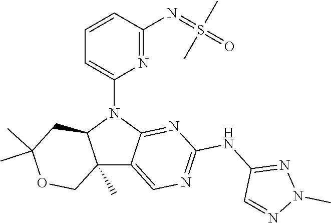 | 515 | 1H NMR (300 MHz, DMSO-d6) δ 9.88 (s, 1H), 8.07 (d, J = 7.8 Hz, 1H), 8.02 (s, 1H), 7.89 (s, 1H), 7.53 (t, J = 7.8 Hz, 1H), 6.35-6.27 (m, 1H), 4.58-4.47 (m, 1H), 4.04 (s, 3H), 3.87-3.79 (m, 1H), 3.69-3.61 (m, 1H), 3.47 (s, 3H), 3.29 (s, 3H), 2.48-2.40 (m, 1H), 1.58-1.45 (m, 1H), 1.27 (s, 3H), 1.22 (s, 3H), 1.01 (s, 3H). | LCMS (ES, m/z): 484.15 [M + H]+, Rt 1.098 min. |
| 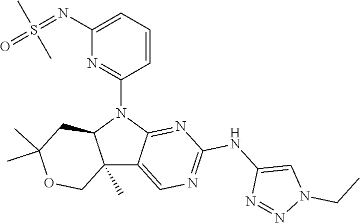 [SMILES: CCn1cc(Nc2ncc3c(n2)N(c2cccc(N=S(C)(C)=O)n2)[C@@H]2CC(C)(C)OC[C@]32C)nn1] | 516 | 1H NMR (400 MHz, DMSO-d6) δ 9.84 (s, 1H), 8.09 (s, 2H), 8.00 (s, 1H), 7.54 (t, J = 8.0 Hz, 1H), 6.33 (d, J = 7.6 Hz, 1H), 4.55-4.49 (m, 1H), 4.40-4.32 (m, 2H), 3.87-3.79 (m 1H), 3.72-3.66 (m, 1H), 3.48 (s, 3H), 3.29 (s, 3H), 2.46-2.38 (m, 1H), 1.54-1.48 (m, 1H), 1.44 (t, J = 7.2 Hz, 3H), 1.26 (s, 3H), 1.22 (s, 3H), 1.01 (s, 3H). | LCMS (ES, m/z): 498.10 [M + H]+, Rt 0.853 min. |
| 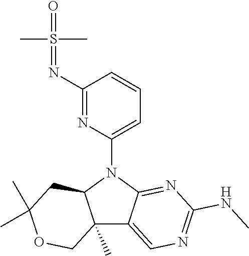 | 723 | 1H NMR (300 MHz, DMSO-d6) δ 8.12 (d, J = 8.1 Hz, 1H), 7.84 (s, 1H), 7.50 (t, J = 7.8 Hz, 1H), 6.77- 6.75 (m, 1H), 6.27 (d, J = 7.8 Hz, 1H), 4.49-4.44 (m, 1H), 3.77-3.58 (m, 2H), 3.47 (s, 3H), 3.28 (s, 3H), 2.79 (d, J = 4.8 Hz, 3H), 2.46-2.40 (m, 1H), 1.44-1.42 (m, 1H), 1.22 (s, 6H), 1.00 (s, 3H). | LCMS (ES, m/z): 417 [M + H]+, Rt 0.572 min. |
| 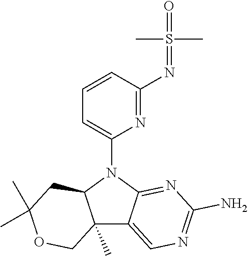 [SMILES: CC1(C)C[C@H]2N(c3cccc(N=S(C)(C)=O)n3)c3nc(N)ncc3[C@@]2(C)CO1] | 746 | 1H NMR (300 MHz, DMSO-d6) δ 8.10 (d, J = 7.8 Hz, 1H), 7.79 (s, 1H), 7.49 (t, J = 7.8 Hz, 1H), 6.29- 6.25 (m, 3H), 4.48-4.44 (m, 1H), 3.78-3.74 (m, 1H), 3.62-3.58 (m, 1H), 3.46 (s, 3H), 3.27 (s, 3H), 2.49-2.39 (m, 1H), 1.47-1.39 (m, 1H), 1.21(s, 3H), 1.19 (s, 3H), 1.01 (s, 3H). | LCMS (ES m/z): 403 [M + H]+, Rt 0.542 min. |

### Examples 219 and 494. Dimethyl((6-((4bR,8aR)-4b,7,7-trimethyl-2-((4-(6-methyl-2,6-diazaspiro[3.3]heptan-2-yl)phenyl)amino)-4b,7,8,8a-tetrahydropyrano[3′,4′:4,5]pyrrolo[2,3-d]pyrimidin-9(5H)-yl)pyridin-2-yl)imino)-l6-sulfanone and ((6-((4bR,8aR)-2-((4-(2,6-diazaspiro[13.3]heptan-2-yl)phenyl)amino)-4b,7,7-trimethyl-4b,7,8,8a-tetrahydropyrano[3′,4′:4,5]pyrrolo[2,3-d]pyrimidin-9(5H)-yl)pyridin-2-yl)imino)dimethyl-l6-sulfanone

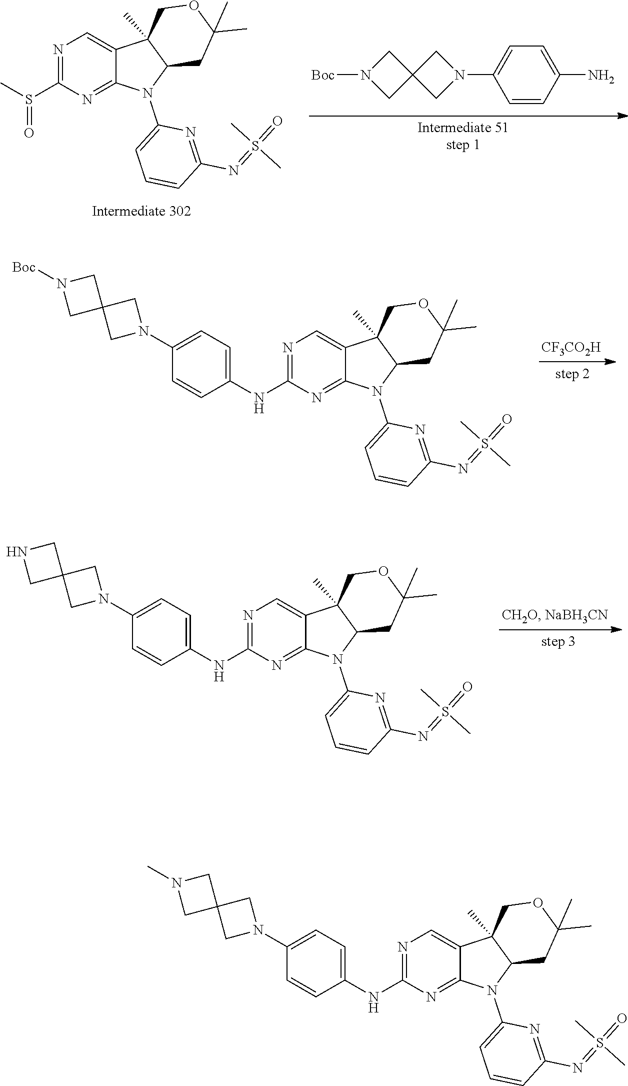

### Step 1. tert-butyl 6-(4-(((4bR,8aR)-9-(6-((dimethyl(oxo)-l6-sulfaneylidene)amino) pyridin-2-yl)-4b,7,7-trimethyl-4b,5,7,8,8a,9-hexahydropyrano[3′,4′:4,5]pyrrolo[2,3-d]pyrimidin-2-yl)amino)phenyl)-2,6-diazaspiro[3.3]heptane-2-carboxylate

To a solution of dimethyl((6-((4bR,8aR)-4b,7,7-trimethyl-2-(methylsulfinyl)-4b,7,8,8a-tetrahydropyrano[3′,4′:4,5]pyrrolo[2,3-d]pyrimidin-9(5H)-yl)pyridin-2-yl)imino)-l6-sulfanone (Intermediate 302, 2.97 g, crude), tert-butyl 6-(4-aminophenyl)-2,6-diazaspiro[3.3]heptane-2-carboxylate (Intermediate 51, 4.78 g, 16.52 mmol, 2.5 eq.) and DIEA (5.75 mL, 33.03 mmol, 5 eq.) in toluene (20 mL) was stirred for 16 h at 100° C. The reaction was monitored by LC-MS. The mixture was allowed to cool down to room temperature. The resulting mixture was concentrated under reduced pressure. The crude product was purified by reverse phase chromatography (Column: C18; Mobile phase, A: water containing 10 mmol/L NH₄HCO₃ and B: ACN (60% to 65% over 10 min); Detector, UV 254 nm, 280 nm) to afford tert-butyl 6-(4-(((4bR,8aR)-9-(6-((dimethyl(oxo)-l6-sulfaneylidene)amino) pyridin-2-yl)-4b,7,7-trimethyl-4b,5,7,8,8a,9-hexahydropyrano[3′,4′:4,5]pyrrolo[2,3-d]pyrimidin-2-yl)amino)phenyl)-2,6-diazaspiro[3.3]heptane-2-carboxylate (3.24 g, 4.80 mmol, 73% yield). LCMS (ES, m/z): 675[M+H]⁺, Rt 0.752 min.

### Step 2. ((6-((4bR,8aR)-2-((4-(2,6-diazaspiro[3.3]heptan-2-yl)phenyl)amino)-4b,7,7-trimethyl-4b,7,8,8a-tetrahydropyrano[3′,4′:4,5]pyrrolo[2,3-d]pyrimidin-9(5H)-yl)pyridin-2-yl)imino)dimethyl-l6-sulfanone (Example 494)

To a stirred solution of tert-butyl 6-(4-(((4bR,8aR)-9-(6-((dimethyl(oxo)-l6-sulfaneylidene)amino)pyridin-2-yl)-4b,7,7-trimethyl-4b,5,7,8,8a,9-hexahydropyrano[3′,4′:4,5]pyrrolo[2,3-d]pyrimidin-2-yl)amino)phenyl)-2,6-diazaspiro[3.3]heptane-2-carboxylate (3.24 g, 4.80 mmol, 1 eq.) in DCM (20 mL) was added TFA (5 mL) dropwise at 0° C. The resulting mixture was stirred for 2 h at room temperature. The residue was concentrated under reduced pressure to afford ((6-((4bR,8aR)-2-((4-(2,6-diazaspiro[3.3]heptan-2-yl)phenyl)amino)-4b,7, 7-trimethyl-4b,7,8,8a-tetrahydropyrano[3′,4′:4,5]pyrrolo[2,3-d]pyrimidin-9(5H)-yl)pyridin-2-yl)imino)dimethyl-l6-sulfanone (Example 494, 3.54 g, crude) as yellow oil. LCMS (ES, m/z): 575 [M+H]⁺, Rt 0.623 min. ¹H NMR (400 MHz, DMSO-d6) δ 8.94 (s, 1H), 8.09 (d, J=8.0 Hz, 1H), 7.94 (s, 1H), 7.69-7.35 (m, 3H), 6.60-6.19 (m, 3H), 4.65-4.35 (m, 1H), 4.01-3.75 (m, 6H), 3.75-3.59 (m, 4H), 3.58-3.42 (m, 4H), 3.31 (s, 3H), 2.49-2.41 (m, 1H), 1.65-1.45 (m, 1H), 1.28 (s, 3H), 1.23 (s, 3H), 1.01 (s, 3H).

### Step 3. dimethyl((6-((4bR,8aR)-4b,7,7-trimethyl-2-((4-(6-methyl-2,6-diazaspiro[3.3]heptan-2-yl)phenyl)amino)-4b,7,8,8a-tetrahydropyrano[3′,4′:4,5]pyrrolo[2,3-d]pyrimidin-9(5H)-yl)pyridin-2-yl)imino)-l6-sulfanone (Example 219)

To a solution of ((6-((4bR,8aR)-2-((4-(2,6-diazaspiro[3.3]heptan-2-yl)phenyl)amino)-4b,7,7-trimethyl-4b,7,8,8a-tetrahydropyrano[3′,4′:4,5]pyrrolo[2,3-d]pyrimidin-9(5H)-yl)pyridin-2-yl)imino)dimethyl-l6-sulfanone (3.54 g, crude) in EtOH (15 mL) was added HCHO (5.00 mL, 61.59 mmol, 10 eq., 37% in water) and HOAc (5.29 mL, 92.39 mmol, 15 eq.). The mixture was stirred for 5 min at 0° C. To above solution was added NaBH₃CN (3.87 g, 61.59 mmol, 10 eq.) at 0° C. The mixture was stirred for additional 2 h at room temperature. The reaction was monitored by LC-MS. The resulting mixture was concentrated under reduced pressure. The residue was purified by reverse phase chromatography (Column: C18; Mobile phase, A: water containing 0.1% FA and Mobile phase B: ACN (0% to 50% over 40 min); Detector, UV 254 nm, 280 nm). The fraction was concentrated under reduced pressure. Then the crude product was purified by reverse phase chromatography (Column: C18; Mobile phase A: water containing 10 mmol/L NH₄HCO₃+0.1% NH₃·H₂O and Mobile phase B: ACN (40% to 50% over 10 min); Detector, UV 254 nm) to afford dimethyl((6-((4bR,8aR)-4b,7,7-trimethyl-2-((4-(6-methyl-2,6-diazaspiro[3.3]heptan-2-yl)phenyl)amino)-4b,7,8,8a-tetrahydropyrano[3′,4′:4,5]pyrrolo[2,3-d]pyrimidin-9(5H)-yl)pyridin-2-yl)imino)-l6-sulfanone (1.16 g, 1.90 mmol, 32% yield) as a white solid.

¹H NMR (300 MHz, DMSO-d₆) δ 8.92 (s, 1H), 8.08 (d, J=8.1 Hz, 1H), 7.93 (s, 1H), 7.53-7.45 (m, 3H), 6.38 (d, J=8.7 Hz, 2H), 6.30 (d, J=7.2 Hz, 1H), 4.53-4.48 (m, 1H), 3.82-3.79 (m, 5H), 3.64-3.47 (m, 5H), 3.29 (s, 6H), 2.49-2.40 (m, 1H), 2.22 (s, 3H), 1.52-1.44 (m, 1H), 1.25 (s, 3H), 1.22 (s, 3H), 1.01 (s, 3H). LCMS (ES, m/z): 589 [M+H]⁺, Rt 0.570 min.

### Example 229. Dimethyl((6-((4bR,8aR)-4b,7,7-trimethyl-2-((1-(1-methylpiperidin-4-yl)-1H-pyrazol-3-yl)amino)-4b,7,8,8a-tetrahydropyrano[3′,4′:4,5]pyrrolo[2,3-d]pyrimidin-9(5H)-yl)pyridin-2-yl)imino)-l6-sulfanone

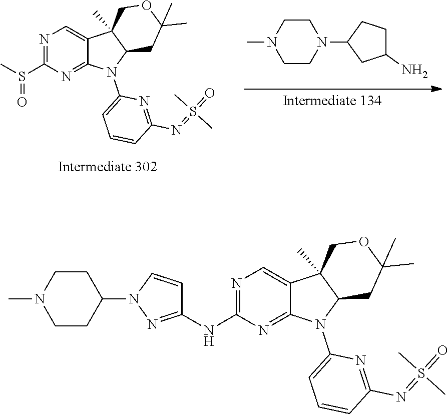

To a stirred mixture of dimethyl((6-((4bR,8aR)-4b,7,7-trimethyl-2-(methylsulfinyl)-4b,7,8,8a-tetrahydropyrano[3′,4′:4,5]pyrrolo[2,3-d]pyrimidin-9(5H)-yl)pyridin-2-yl)imino)-l6-sulfanone (Intermediate 302, 80 mg, 177.94 μmol, 1 eq.) and 1-(1-methyl-4-piperidyl) pyrazol-3-amine (Intermediate 134, 64.15 mg, 355.88 μmol, 2 eq.) in o-xylene (1 mL) were added CuMeSal (83.65 mg, 391.47 μmol, 2.2 eq.), Pd(OAc)₂ (3.99 mg, 17.79 μmol, 0.1 eq.) and Cs₂CO₃ (173.93 mg, 533.82 μmol, 3 eq.) at room temperature under N₂ atmosphere. The resulting mixture was stirred for 16 h at 100° C. under N₂ atmosphere. The reaction was monitored by LCMS. The mixture was allowed to cool down to room temperature. The resulting mixture was filtered. The filter cake was washed with DCM (3×10 mL). The filtrate was concentrated under reduced pressure. The resulting mixture was concentrated under vacuum. The crude product was purified by Prep-HPLC with the following conditions (Column: XBridge Prep OBD C18 Column, 30*150 mm, 5 μm; Mobile Phase A: Water(10 mmol/L NH₄HCO₃+0.1% NH₃·H₂O), Mobile Phase B: ACN; Flow rate: 60 mL/min; Gradient: 20% B to 50% B in 7 min, 50% B;) to afford dimethyl((6-((4bR,8aR)-4b,7,7-trimethyl-2-((1-(1-methylpiperidin-4-yl)-1H-pyrazol-3-yl)amino)-4b,7,8,8a-tetrahydropyrano[3′,4′:4,5]pyrrolo[2,3-d]pyrimidin-9(5H)-yl)pyridin-2-yl)imino)-l6-sulfanone (12.7 mg, 22.43 μmol, 13% yield) as an off-white solid. ¹H NMR (400 MHz, DMSO-d6) δ 8.97 (s, 1H), 8.10-7.80 (m, 2H), 7.60-7.30 (m, 2H), 6.29 (d, J=8.0 Hz, 1H), 6.16 (s, 1H), 4.67-4.35 (m, 1H), 4.28-4.00 (m, 1H), 3.95-3.75 (m, 1H), 3.75-3.61 (m, 1H), 3.47 (s, 3H), 3.29 (s, 3H), 3.00-2.70 (m, 2H), 2.49-2.41 (m, 1H), 2.16 (s, 3H), 2.10-1.85 (m, 4H), 1.85-1.68 (m, 2H), 1.68-1.40 (m, 1H), 1.28 (s, 3H), 1.24 (s, 3H), 1.01 (s, 3H). LCMS (ES, m/z): 566 [M+H]⁺, Rt 0.532 min.

TABLE 19
The examples in the following table were synthesized according to the
appropriate method as demonstrated in Example 229.

| Structure | Ex. # | ¹H NMR | LCMS |
|---|---|---|---|
| 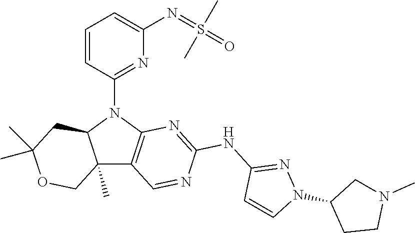 | 225 | 1H NMR (400 MHz, DMSO-d6) δ 10.70 (br, 1H), 8.00 (s, 2H), 7.49 (d, J = 8.0 Hz, 1H), 7.28 (s, 1H), 6.41 (s, 1H), 6.31 (d, J = 7.6 Hz, 1H), 5.18-4.88 (m, 1H), 4.67-4.40 (m, 1H), 3.85-3.81 (m, 1H), 3.67-3.62 (m, 1H), 3.48 (s, 3H), 3.29 (s, 3H), 3.01-2.92 (m, 2H), 2.85-2.82 (m, 1H), 2.45-2.29 (m, 6H), 2.05- 1.91 (m, 1H), 1.69-1.41 (m, 1H), 1.28 (s, 3H), 1.22 (s, 3H), 1.01 (s, 3H). | LCMS (ES, m/z): 552 [M + H]+, Rt 0.541 min. |
| 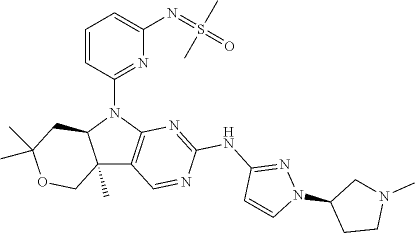 | 227 | 1H NMR (400 MHz, DMSO-d6) δ 10.78 (br, 1H), 8.01 (s, 2H), 7.49 (t, J = 8.0 Hz, 1H), 7.28 (s, 1H), 6.44 (s, 1H), 6.32 (d, J = 7.6 Hz, 1H), 5.08-5.01 (m, 1H), 4.55-4.51 (m, 1H), 3.84-3.80 (m, 1H), 3.66-3.62 (m, 1H), 3.48 (s, 3H), 3.29 (s, 3H), 3.11-2.90 (m, 2H), 2.70 (t, J = 8.8 Hz, 1H), 2.47-2.34 (m, 6H), 2.07- 1.92 (m, 1H), 1.55-1.45 (m, 1H), 1.25(s, 3H), 1.20 (s, 3H), 1.02 (s, 3H). | LCMS (ES, m/z): 552 [M + H]+, Rt 0.527 min. |
| 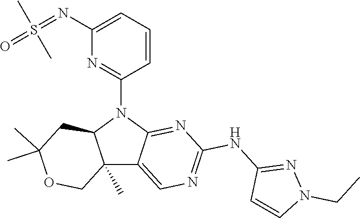 [SMILES: CCn1ccc(Nc2ncc3c(n2)N(c2cccc(N=S(C)(C)=O)n2)[C@@H]2CC(C)(C)OC[C@]32C)n1] | 830 | 1H NMR (400 MHz, DMSO-d6) δ 9.37 (s, 1H), 8.17 (d, J = 8.0 Hz, 1H), 7.97 (s, 1H), 7.57 (d, J = 2.0 Hz, 1H), 7.49 (t, J = 8.0 Hz, 1H), 6.49 (d, J = 1.6 Hz, 1H), 6.30 (d, J = 7.6 Hz, 1H), 4.53-4.49 (m, 1H), 4.06-3.97 (m, 2H), 3.81-3.63 (m, 2H), 3.48 (s, 3H), 3.29 (s, 3H), 2.47-2.41 (m, 1H), 1.53-1.46 (m, 1H), 1.36 (t, J = 7.2 Hz, 3H), 1.25 (s, 3H), 1.23 (s, 3H), 1.01 (s, 3H). | LCMS (ES, m/z): 497.25 [M + H]+, Rt 0.675 min. |
| 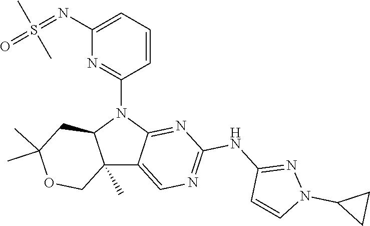 [SMILES: CC1(C)C[C@H]2N(c3cccc(N=S(C)(C)=O)n3)c3nc(Nc4ccn(C5CC5)n4)ncc3[C@@]2(C)CO1] | 831 | 1H NMR (400 MHz, DMSO-d6) δ 9.37 (s, 1H), 8.16 (d, J = 8.0 Hz, 1H), 7.97 (s, 1H), 7.62 (d, J = 2.4 Hz, 1H), 7.50 (t, J = 8.0 Hz, 1H), 6.50 (d, J = 2.4 Hz, 1H), 6.31 (d, J = 7.6 Hz, 1H), 4.52-4.45 (m, 1H), 3.87-3.82 (m, 1H), 3.68-3.54 (m, 2H), 3.48 (s, 3H), 3.38 (s, 3H), 2.47-2.42 (m, 1H), 1.50-1.45 (m, 1H), 1.27 (s, 3H), 1.24 (s, 3H), 1.04-0.92 (m, 3H), 0.95-0.82 (m, 4H). | LCMS (ES, m/z): 509.10 [M + H]+, Rt 0.942 min. |
| 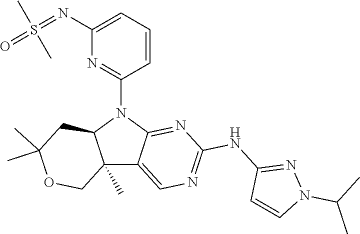 [SMILES: CC(C)n1ccc(Nc2ncc3c(n2)N(c2cccc(N=S(C)(C)=O)n2)[C@@H]2CC(C)(C)OC[C@]32C)n1] | 832 | 1H NMR (300 MHz, DMSO-d6) δ 9.28 (s, 1H), 8.10 (d, J = 8.4 Hz, 1H), 7.90 (s, 1H), 7.53 (d, J = 2.4 Hz, 1H), 7.42 (t, J = 8.1 Hz, 1H), 6.42 (d, J = 2.7 Hz, 1H), 6.23 (d, J = 7.8 Hz, 1H), 4.69-4.42 (m, 1H), 4.33-4.28 (m, 1H), 3.75-3.56 (m, 2H), 3.41 (s, 3H), 3.26 (s, 3H), 2.43 (d, J = 1.8 Hz, 1H), 1.49-1.41 (m, 1H), 1.34 (d, J = 6.6 Hz, 6H), 1.19 (d, J = 9.0 Hz, 6H), 1.01 (s, 3H). | LCMS (ES, m/z): 511.25 [M + H]+, Rt 0.725 min. |

### Intermediate 303. ((6-((4bR,8aR)-2-amino-4b,7,7-trimethyl-4b,7,8,8a-tetrahydropyrano[3′,4′:4,5]pyrrolo[2,3-d]pyrimidin-9(5H)-yl)pyridin-2-yl)imino)dimethyl-l6-sulfanone

### Step 1. ((6-((4bR,8aR)-2-((2,4-dimethoxybenzyl)amino)-4b,7,7-trimethyl-4b,7,8,8a-tetrahydropyrano[3′,4′:4,5]pyrrolo[2,3-d]pyrimidin-9(5H)-yl)pyridin-2-yl)imino)dimethyl-l6-sulfanone

A mixture of dimethyl((6-((4bR,8aR)-4b,7,7-trimethyl-2-(methylsulfinyl)-4b,7,8,8a-tetrahydropyrano[3′,4′:4,5]pyrrolo[2,3-d]pyrimidin-9(5H)-yl)pyridin-2-yl)imino)-l6-sulfanone (Intermediate 302, 1.40 g, crude), (2,4-dimethoxyphenyl)methanamine (1.56 g, 9.34 mmol, 2.5 eq.) and DIEA (2.23 g, 17.22 mmol, 5 eq.) in toluene (8 mL) was stirred for 16 h at 100° C. The reaction was monitored by LC-MS. The mixture was allowed to cool down to room temperature. The resulting mixture was concentrated under reduced pressure. The crude product was purified by reverse phase chromatography (Column: C18; Mobile phase, A: water containing 10 mmol/L NH4HCO3 and B: ACN (60% to 65% over 10 min); Detector, UV 254/280 nm). The product was concentrated under reduced pressure to afford ((6-((4bR,8aR)-2-((2,4-dimethoxybenzyl)amino)-4b,7,7-trimethyl-4b,7,8,8a-tetrahydropyrano[3′,4′:4,5]pyrrolo[2,3-d]pyrimidin-9(5H)-yl)pyridin-2-yl)imino) dimethyl-l6-sulfanone (1.6 g, 2.89 mmol, 88% yield). LCMS (ES, m/z): 553 [M+H]+, Rt 0.532 min.

### Step 2. ((6-((4bR,8aR)-2-amino-4b,7,7-trimethyl-4b,7,8,8a-tetrahydropyrano[3′,4′:4,5]pyrrolo[2,3-d]pyrimidin-9(5H)-yl)pyridin-2-yl)imino)dimethyl-l6-sulfanone (Intermediate 303)

A mixture of ((6-((4bR,8aR)-2-((2,4-dimethoxybenzyl)amino)-4b,7,7-trimethyl-4b,7,8,8a-tetrahydropyrano[3′,4′:4,5]pyrrolo[2,3-d]pyrimidin-9(5H)-yl)pyridin-2-yl)imino)dimethyl-l6-sulfanone (1.6 g, 2.89 mmol, 1 eq.) in TFA (10 mL) was stirred for 16 h at room temperature. The reaction was monitored by LC-MS. The mixture was concentrated under reduced pressure. Then saturated sodium bicarbonate (100 mL) was added. The resulting mixture was extracted with EA (3×100 mL). The combined organic phase was dried over anhydrous Na₂SO₄. After filtration, the filtrate was concentrated under reduced pressure. The residue was purified by silica gel column chromatography (eluting with 10:1 DCM/EtOH) to afford ((6-((4bR,8aR)-2-amino-4b,7,7-trimethyl-4b,7,8,8a-tetrahydropyrano[3′,4′:4,5]pyrrolo[2,3-d]pyrimidin-9(5H)-yl)pyridin-2-yl)imino)dimethyl-l6-sulfanone (1.02 g, 2.42 mmol, 84% yield) as a white solid. LCMS (ES, m/z): 403 [M+H]⁺, Rt 0.502 min.

### Example 233: 4-[[(1R,9R)-8-[6-[[dimethyl(oxo)-sulfanylidene]amino]-2-pyridyl]-1,11,11-trimethyl-12-oxa-4,6,8-triazatricyclo[7.4.0.02,7]trideca-2,4,6-trien-5-yl]amino]-1-tetrahydropyran-4-yl-pyridin-2-one

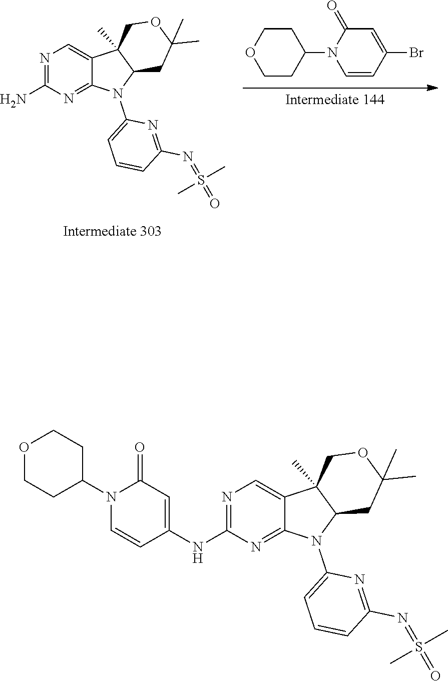 [SMILES: CC1(C)C[C@H]2N(c3cccc(N=S(C)(C)=O)n3)c3nc(N)ncc3[C@@]2(C)CO1 ; O=c1cc(Br)ccn1C1CCOCC1 ; CC1(C)C[C@H]2N(c3cccc(N=S(C)(C)=O)n3)c3nc(Nc4ccn(C5CCOCC5)c(=O)c4)ncc3[C@@]2(C)CO1]

To a stirred mixture of (1R,9R)-8-[6-[[dimethyl(oxo)-sulfanylidene]amino]-2-pyridyl]-1,11,11-trimethyl-12-oxa-4,6,8-triazatricyclo[7.4.0.02,7]trideca-2,4,6-trien-5-amine (Intermediate 303, 40 mg, 99.38 μmol, 1 eq.) and 4-bromo-1-tetrahydropyran-4-yl-pyridin-2-one (30.78 mg, 119.25 μmol, 1.2 eq.) in dioxane (2 mL) were added Xphos (9.46 mg, 19.88 μmol, 0.2 eq.), Xphos Pd G3 (8.41 mg, 9.94 μmol, 0.1 eq.) and Cs₂CO₃ (97.14 mg, 298.13 μmol, 3 eq.) at room temperature under nitrogen atmosphere. The resulting mixture was stirred for 2 h at 100° C. under nitrogen atmosphere. The mixture was allowed to cool down to room temperature. The resulting mixture was concentrated under reduced pressure. The residue was purified by Prep-TLC (eluting with 1:2 EA/PE). The crude product was purified by prep-HPLC (Column: XBridge Prep OBD C18 Column, 30×150 mm, 5 μm; Mobile Phase A: Water (10 mmol/L NH₄HCO₃+0.1% NH₃·H₂O), Mobile Phase B: ACN; Flow rate: 60 mL/min; Gradient: 20% B to 50% B in 7 min, 50% B; Wave Length: 254 nm;) to afford 4-[[(1R,9R)-8-[6-[[dimethyl(oxo)-sulfanylidene]amino]-2-pyridyl]-1,11,11-trimethyl-12-oxa-4,6,8-triazatricyclo[7.4.0.02,7]trideca-2,4,6-trien-5-yl]amino]-1-tetrahydropyran-4-yl-pyridin-2-one (28.1 mg, 48.38 μmol, 49% yield) as a white solid. LCMS (ES, m/z): 580 [M+H]⁺, Rt 0.606 min. ¹H NMR (400 MHz, DMSO-d6) δ 9.56 (s, 1H), 8.11 (s, 1H), 8.05 (d, J=8.0 Hz, 1H), 7.85-7.28 (m, 2H), 7.10 (d, J=2.4 Hz, 1H), 6.57 (d, J=2.4 Hz, 1H), 6.35 (d, J=8.0 Hz, 1H), 4.95-4.72 (m, 1H), 4.58-4.35 (m, 1H), 4.09-3.90 (m, 2H), 3.90-3.75 (m, 1H), 3.75-3.65 (m, 1H), 3.55-3.36 (m, 5H), 3.29 (s, 3H), 2.48-2.39 (m, 1H), 1.92-1.78 (m, 2H), 1.69-1.52 (m, 2H), 1.58-1.48 (m, 1H), 1.28 (s, 3H), 1.22 (s, 3H), 1.01 (s, 3H). LCMS (ES, m/z): 580 [M+H]+, Rt 0.606 min.

### Example 361: ((6-((4bR,8aR)-2-(imidazo[1,2-b]pyridazin-6-ylamino)-4b,7,7-trimethyl-4b,7,8,8a-tetrahydropyrano[3′,4′:4,5]pyrrolo[2,3-d]pyrimidin-9(5H)-yl)pyridin-2-yl)imino)dimethyl-l6-sulfanone

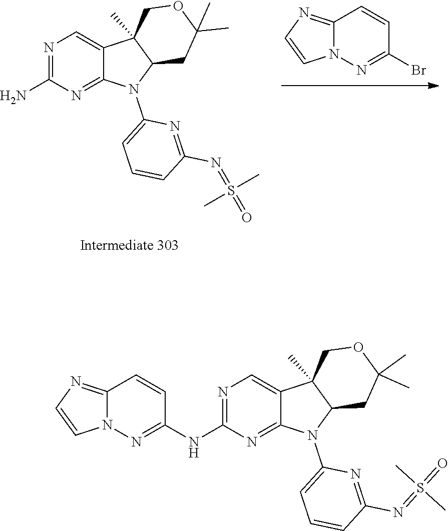 [SMILES: CC1(C)C[C@H]2N(c3cccc(N=S(C)(C)=O)n3)c3nc(N)ncc3[C@@]2(C)CO1 ; Brc1ccc2nccn2n1 ; CC1(C)C[C@H]2N(c3cccc(N=S(C)(C)=O)n3)c3nc(Nc4ccc5nccn5n4)ncc3[C@@]2(C)CO1]

Into a mixture of (1R,9R)-8-[6-[[dimethyl(oxo)-sulfanylidene]amino]-2-pyridyl]-1,11,11-trimethyl-12-oxa-4,6,8-triazatricyclo[7.4.0.0^{2,7}]trideca-2,4,6-trien-5-amine (Intermediate 303, 50 mg, 124.22 μmol, 1 eq.) and Intermediate 87 (29.52 mg, 149.06 μmol, 1.2 eq.) in dioxane (2 mL) were added Xphos Pd G3 (10.51 mg, 12.42 μmol, 0.1 eq.) and Xphos (11.84 mg, 24.84 μmol, 0.2 eq.) and Cs₂CO₃ (80.95 mg, 248.44 μmol, 2 eq.). The mixture was stirred for 2 h at 100° C. under N₂ atmosphere. The reaction was monitored by LCMS. The mixture was allowed to cool down to room temperature. The solids were filtered out and washed with EA (3×5 mL). The filtrate was concentrated under reduced pressure. The residue was purified by prep-HPLC (Column: XBridge Prep OBD C18 Column, 30*150 mm, 5 μm; Mobile Phase A: Water (10 mmol/L NH₄HCO₃+0.1% NH₃·H₂O), Mobile Phase B: ACN; Flow rate: 60 mL/min; Gradient: 22% B to 44% B in 7 min, 44% B; Wave Length: 254 nm) to afford ((6-((4bR,8aR)-2-(imidazo[1,2-b]pyridazin-6-ylamino)-4b,7,7-trimethyl-4b,7,8,8a-tetrahydropyrano[3′,4′:4,5]pyrrolo[2,3-d]pyrimidin-9(5H)-yl)pyridin-2-yl)imino)dimethyl-l6-sulfanone (22.7 mg, 43.6 μmol, 35% yield) as a white solid. LCMS (ES, m/z): 520 [M+H]+; RT: 0.989 min. ¹H NMR (300 MHz, DMSO-d6) δ 10.16 (s, 1H), 8.24 (d, J=8.1 Hz, 1H), 8.10 (s, 1H), 8.08-8.00 (m, 2H), 7.81 (d, J=9.9 Hz, 1H), 7.65 (s, 1H), 7.56 (t, J=7.8 Hz, 1H), 6.34 (d, J=7.8 Hz, 1H), 4.63-4.52 (m, 1H), 3.87-3.82 (m, 1H), 3.66-3.61 (m, 1H), 3.50 (s, 3H), 3.30 (s, 3H), 2.50-2.46 (m, 1H), 1.61-1.47 (m, 1H), 1.30 (s, 3H), 1.24 (s, 3H), 1.03 (s, 3H).

### Example 362: ((6-((4bR,8aR)-2-([1,2,3]triazolo[1,5-a]pyridin-6-ylamino)-4b,7,7-trimethyl-4b,7,8,8a-tetrahydropyrano[3′,4′:4,5]pyrrolo[2,3-d]pyrimidin-9(5H)-yl)pyridin-2-yl)imino)dimethyl-l6-sulfanone

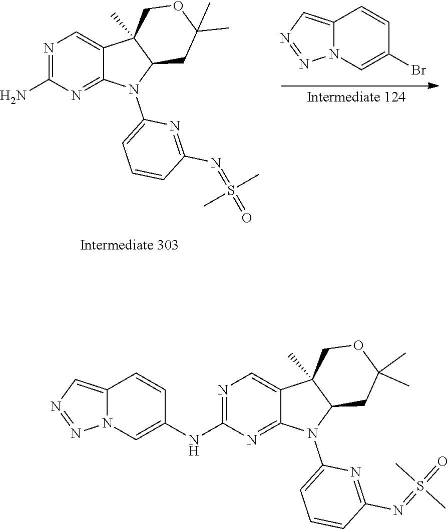 [SMILES: CC1(C)C[C@H]2N(c3cccc(N=S(C)(C)=O)n3)c3nc(N)ncc3[C@@]2(C)CO1 ; Brc1ccc2cnnn2c1 ; CC1(C)C[C@H]2N(c3cccc(N=S(C)(C)=O)n3)c3nc(Nc4ccc5cnnn5c4)ncc3[C@@]2(C)CO1]

To a stirred mixture of ((6-((4bR,8aR)-2-amino-4b,7,7-trimethyl-4b,7,8,8a-tetrahydropyrano[3′,4′:4,5]pyrrolo[2,3-d]pyrimidin-9(5H)-yl)pyridin-2-yl)imino)dimethyl-l6-sulfanone (Intermediate 303, 50 mg, 124.22 μmol, 1 eq.) in dioxane (2 mL) was added Intermediate 124 (29.52 mg, 149.06 μmol, 1.2 eq.), XantPhos (7.2 mg, 12.4 μmol, 0.1 eq.), XantPhos Pd G3 (11.0 mg, 12.4 μmol, 0.1 eq.) and Cs₂CO₃ (101.2 mg, 310.6 μmol, 2.5 eq.). The resulting mixture was stirred for 16 h at 100° C. under N2 atmosphere. The reaction was monitored by LCMS. The mixture was cooled down to room temperature. The solids were filtered out and washed by EA (5 mL×3). The resulting mixture was concentrated under reduced pressure. The crude product was purified by Prep-HPLC (Column: XBridge Prep OBD C18 Column, 30×150 mm, 5 μm; Mobile Phase A: Water(10 mmol/L NH₄HCO₃+0.1% NH₃·H₂O), Mobile Phase B: ACN (25% to 55% in 7 min); Flow rate: 60 mL/min; Wave Length: 254 nm) to afford ((6-((4bR,8aR)-2-([1,2,3]triazolo[1,5-a]pyridin-6-ylamino)-4b,7,7-trimethyl-4b,7,8,8a-tetrahydropyrano[3′,4′:4,5]pyrrolo[2,3-d]pyrimidin-9(5H)-yl)pyridin-2-yl)imino)dimethyl-l6-sulfanone (22.4 mg, 42.7 μmol, 34% yield) as a white solid. LCMS (ES, m/z): 520 [M+H]⁺, Rt 0.976 min. ¹H NMR (400 MHz, DMSO-d6) δ 9.93 (s, 1H), 9.79 (s, 1H), 8.19-8.07 (m, 3H), 7.88 (d, J=9.2 Hz, 1H), 7.63-7.52 (m, 2H), 6.39 (d, J=7.6 Hz, 1H), 4.59-4.52 (m, 1H), 3.89-3.86 (m, 1H), 3.70-3.67 (m, 1H), 3.50 (s, 3H), 3.32 (s, 3H), 2.48-2.44 (m, 1H), 1.60-1.54 (m, 1H), 1.31 (s, 3H), 1.24 (s, 3H), 1.03 (s, 3H).

Using appropriate intermediates and reaction conditions, the following examples in Table 20 were prepared in the similar manner to Examples 233/361/362.

TABLE 20

| Structure | EX # | ¹H NMR | LCMS |
|---|---|---|---|
| 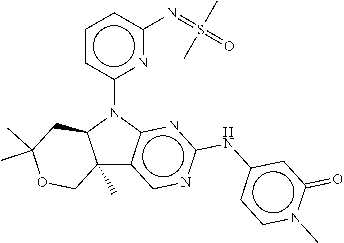 | 363 | 1H NMR (400 MHz, DMSO-d6) δ 9.56 (s, 1H), 8.12 (s, 1H), 8.05 (d, J = 7.6 Hz, 1H), 7.57 (t, J = 8.0 Hz, 1H), 7.50 (d, J = 7.6 Hz, 1H), 7.10 (d, J = 2.4 Hz, 1H), 6.52-6.36 (m, 1H), 6.35 (d, J = 7.2 Hz, 1H), 4.58- 4.55 (m, 1H), 3.87-3.82 (m, 1H), 3.66-3.61 (m, 1H), 3.49 (s, 3H), 3.33 (s, 3H), 3.30 (s, 3H), 2.50- 2.42 (m, 1H), 1.57-1.51 (m, 1H), 1.28 (s, 3H), 1.23 (s, 3H), 1.01 (s, 3H). | LCMS (ES, m/z): 510 [M + H]+, Rt 0.584 min. |
| 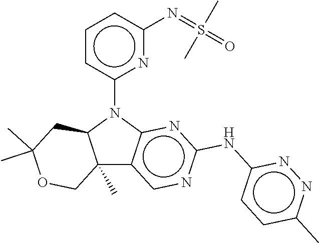 | 364 | 1H NMR (400 MHz, DMSO-d6) δ 8.12-8.05 (m, 3H), 7.67-7.60 (m, 2H), 6.44 (d, J = 8.0 Hz, 1H), 4.67- 4.64 (m, 1H), 3.89-3.85 (m, 1H), 3.70-3.66 (m, 1H), 3.49 (s, 3H), 3.31 (s, 3H), 2.58 (s, 3H), 2.47- 2.42 (m, 1H), 1.64-1.58 (m, 1H), 1.32 (s, 3H), 1.22 (s, 3H), 1.01 (s, 3H). | LCMS (ES, m/z): 495 [M + H]+, Rt 0.642 min. |
| 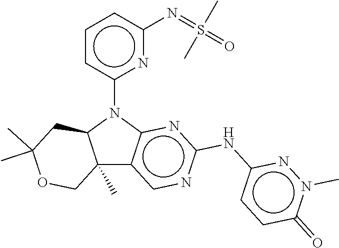 | 365 | 1H NMR (400 MHz, DMSO-d6) δ 9.71 (s, 1H), 8.06 (d, J = 8.0 Hz, 1H), 8.00 (s, 1H), 7.93 (d, J = 10.0 Hz, 1H), 7.52 (t, J = 8.0 Hz, 1H), 6.95 (d, J = 9.6 Hz, 1H), 6.32 (d, J = 7.6 Hz, 1H), 4.57-4.53 (m, 1H), 3.87-3.82 (m, 1H), 3.66-3.61 (m, 1H), 3.59 (s, 3H), 3.48 (s, 3H), 3.29 (s, 3H), 2.50-2.42 (m, 1H), 1.54-1.50 (m, 1H), 1.26 (s, 3H), 1.23 (s, 3H), 1.01 (s, 3H). | LCMS (ES, m/z): 511 [M + H]+, Rt 0.594 min. |
| 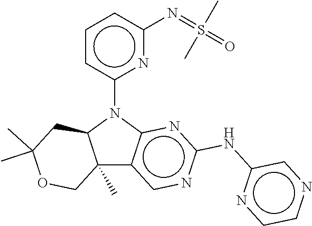 | 366 | 1H NMR (400 MHz, DMSO-d6) δ 9.99 (s, 1H), 9.49 (s, 1H), 9.31 (s, 1H), 8.18-8.12 (m, 3H), 7.53 (t, J = 8.0 Hz, 1H), 6.35 (d, J = 8.0 Hz, 1H), 4.59-4.55 (m, 1H), 3.87- 3.84 (m, 1H), 3.68-3.65 (m, 1H), 3.48 (s, 3H), 3.29 (s, 3H), 2.46- 2.43 (m, 1H), 1.57-1.51 (m, 1H), 1.29 (s, 3H), 1.23 (s, 3H), 1.01 (s, 3H). | LCMS (ES, m/z): 481 [M + H]+, Rt 0.623 min. |
| 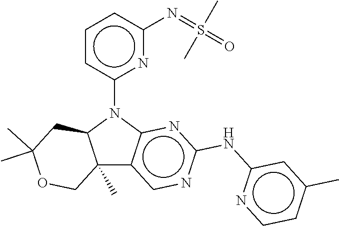 | 367 | 1H NMR (300 MHz, DMSO-d6) δ 10.60 (s, 1H), 8.30 (d, J = 7.8 Hz, 1H), 8.13 (s, 1H), 7.53-7.47 (m, 2H), 7.26 (s, 1H), 6.32 (d, J = 7.8 Hz, 1H), 4.59-4.54 (m, 1H), 3.87- 3.83 (m, 1H), 3.75-3.64 (m, 4H), 3.48 (s, 3H), 3.32 (s, 3H), 2.44- 2.42 (m, 1H), 2.32-2.11 (m, 2H), 1.55-1.51 (m, 1H), 1.48-1.40 (m, 1H), 1.27 (s, 3H), 1.22 (s, 3H), 1.19-1.12 (m, 1H), 1.00 (s, 3H). | LCMS (ES, m/z): 551 [M + H]+, Rt 1.227 min. |
| 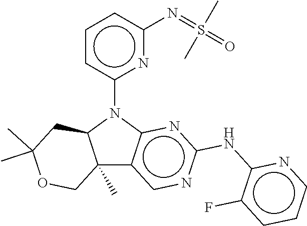 | 368 | 1H NMR (400 MHz, DMSO-d6) δ 9.48 (s, 1H), 8.12 (d, J = 4.8 Hz, 1H), 7.97 (s, 1H), 7.87 (d, J = 8.0 Hz, 1H), 7.71 (t, J = 9.6 Hz, 1H), 7.40 (t, J = 8.0 Hz, 1H), 7.20-7.15 (m, 1H), 6.26 (d, J = 8.0 Hz, 1H), 4.65-4.38 (m, 1H), 3.99-3.75 (m, 1H), 3.75-3.59 (m, 1H), 3.47 (s, 3H), 3.27 (s, 3H), 2.43-2.35 (m, 1H), 1.58-1.49 (m, 1H), 1.28 (s, 3H), 1.22 (s, 3H), 1.00 (s, 3H). | LCMS (ES, m/z): 498 [M + H]+, Rt 0.693 min. |
| 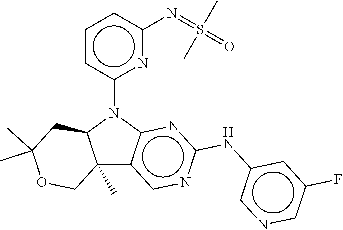 | 369 | 1H NMR (300 MHz, DMSO-d6) δ 9.85 (s, 1H), 8.67 (s, 1H), 8.41- 8.34 (m, 1H), 8.20-8.10 (m, 2H), 8.03 (d, J = 8.1 Hz, 1H), 7.65-7.50 (m, 1H), 6.36 (d, J = 7.8 Hz, 1H), 4.70-4.50 (m, 1H), 3.92-3.85 (m, 1H), 3.75-3.62 (m, 1H), 3.49 (s, 3H), 3.25 (s, 3H), 2.42-2.35 (m, 1H), 1.65-1.45 (m, 1H), 1.29 (s, 3H), 1.23 (s, 3H), 1.02 (s, 3H) | LCMS (ES, m/z): 498 [M + H]+, Rt 0.640 min. |
| 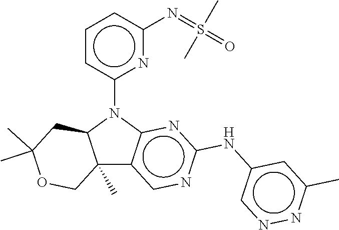 | 370 | 1H NMR (400 MHz, DMSO-d6) δ 10.04 (s, 1H), 9.19 (s, 1H), 8.24- 8.07 (m, 2H), 7.98 (d, J = 8.0 Hz, 1H), 7.62 (t, J = 8.0 Hz, 1H), 6.38 (d, J = 7.6 Hz, 1H), 4.61-4.57(m, 1H), 3.87-3.82 (m, 1H), 3.66-3.61 (m, 1H), 3.49 (s, 3H), 3.31 (s, 3H), 2.53 (s, 3H), 2.46-2.43 (m, 1H), 1.60-1.57 (m, 1H), 1.30 (s, 3H), 1.23 (s, 3H), 1.01 (s, 3H). | LCMS (ES, m/z): 495 [M + H]+, Rt 0.595 min. |
| 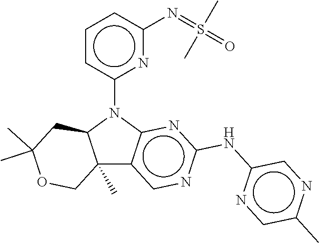 | 371 | 1H NMR (300 MHz, DMSO-d6) δ 9.84 (s, 1H), 9.36 (s, 1H), 8.20- 8.15 (m, 2H), 8.10 (s, 1H), 7.53 (t, J = 7.8 Hz, 1H), 6.35 (d, J = 7.5 Hz, 1H), 4.59-4.54 (m, 1H), 3.87- 3.83 (m, 1H), 3.69-3.64 (m, 1H), 3.49 (s, 3H), 3.29 (s, 3H), 2.50- 2.43 (m, 4H), 1.57-1.49 (m, 1H), 1.29 (s, 3H), 1.23 (s, 3H), 1.01 (s, 3H). | LCMS (ES, m/z): 495 [M + H]+, Rt 0.617 min. |
| 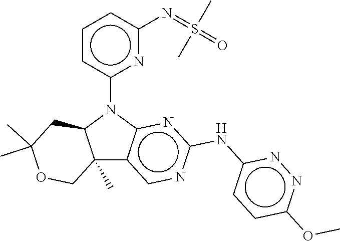 | 372 | 1H NMR (400 MHz, DMSO-d6) δ 9.97 (s, 1H), 8.38 (d, J = 9.6 Hz, 1H), 8.12 (d, J = 8.0 Hz, 1H), 8.04 (s, 1H), 7.56-7.52 (m, 1H), 7.22 (d, J = 9.6 Hz, 1H), 6.33 (d, J = 7.6 Hz, 1H), 4.57-4.454 (m, 1H), 3.98 (s, 3H), 3.87-3.82 (m, 1H), 3.66- 3.61 (m, 1H), 3.48 (s, 3H), 3.32- 3.26(s, 3H), 2.50-2.42 (m, 1H), 1.55-1.49 (m, 1H), 1.27 (s, 3H), 1.24 (s, 3H), 1.01 (s, 3H). | LCMS (ES, m/z): 511 [M + H]+, Rt 1.253 min. |
| 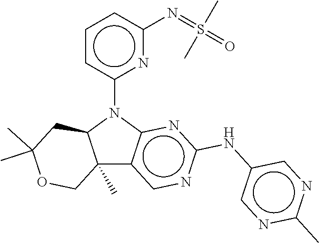 | 373 | 1H NMR (300 MHz, DMSO-d6) δ 9.56 (s, 1H), 9.05 (s, 2H), 8.08 (s, 1H), 8.02 (d, J = 8.1 Hz, 1H), 7.55 (t, J = 8.1 Hz, 1H), 6.35 (d, J = 7.8 Hz, 1H), 4.61-4.50 (m, 1H), 3.87- 3.82 (m, 1H), 3.66-3.61 (m, 1H), 3.49 (s, 3H), 3.31 (s, 3H), 2.55 (s, 3H), 2.50-2.40 (m, 1H), 1.59-1.45 (m, 1H), 1.28 (s, 3H), 1.23 (s, 3H), 1.02 (s, 3H). | LCMS (ES, m/z): 495 [M + H]+; RT: 0.608 min. |
| 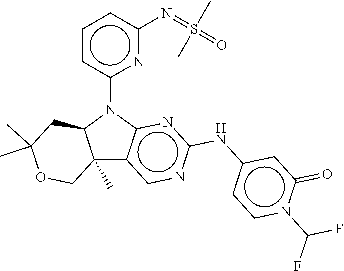 | 374 | 1H NMR (300 MHz, DMSO-d6) δ 9.92 (s, 1H), 8.16 (s, 1H), 8.02- 7.97 (m, 1H), 7.77-7.54 (m, 3H), 7.27 (d, J = 2.1 Hz, 1H), 6.66-6.63 (m, 1H), 6.36 (d, J = 7.5 Hz, 1H), 4.60-4.56 (m, 1H), 3.88-3.84 (m, 1H), 3.68-3.64 (m, 1H), 3.48 (s, 3H), 3.30 (s, 3H), 2.49-2.40 (m, 1H), 1.60-1.52 (m, 1H), 1.29 (s, 3H), 1.22 (s, 3H), 1.01 (s, 3H). | LCMS (ES, m/z): 546 [M + H]+, Rt 0.680 min. |
| 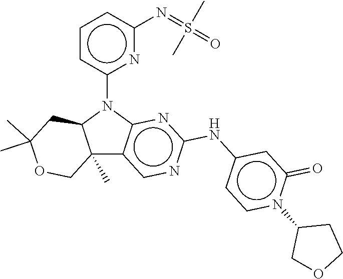 | 375 | 1H NMR (400 MHz, DMSO-d6) δ 9.58 (s, 1H), 8.12 (s, 1H), 8.05 (d, J = 8.0 Hz, 1H), 7.57 (t, J = 8.0 Hz, 1H), 7.42 (d, J = 8.0 Hz, 1H), 7.13 (s, 1H), 6.68-6.51 (m, 1H), 6.38-6.30 (m, 1H), 5.40-5.25 (m, 1H), 4.65-4.35 (m, 1H), 4.11-3.97 (m, 1H), 3.95-3.81 (m, 2H), 3.81- 3.65 (m, 3H), 3.32 (s, 3H), 3.30 (s, 3H), 2.49-2.32 (m, 2H), 2.00-1.85 (m, 1H), 1.62-1.50 (m, 1H), 1.28 (s, 3H), 1.22 (s, 3H), 1.01 (s, 3H). | LCMS (ES, m/z): 566 [M + H]+, Rt 1.232 min. |
| 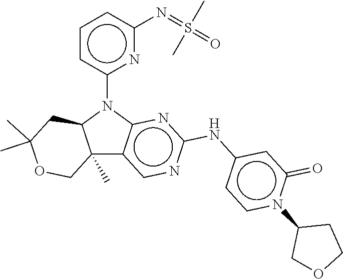 | 376 | 1H NMR (400 MHz, DMSO-d6) δ 9.58 (s, 1H), 8.12 (s, 1H), 8.08- 8.02 (m, 1H), 7.57 (t = 8.0 Hz, 1H), 7.42 (d, J = 8.0 Hz, 1H), 7.13 (s, 1H), 6.62-6.52 (m, 1H), 6.35 (d, J = 7.8 Hz, 1H), 5.42-5.25 (m, 1H), 4.62-4.50 (m, 1H), 4.10- 3.95 (m, 1H), 3.80-3.70 (m, 2H), 3.70-3.62 (m, 3H), 3.45 (s, 3H), 3.27 (s, 3H), 2.49-2.32 (m, 2H), 2.00-1.85 (m, 1H), 1.62-1.45 (m, 1H), 1.28 (s, 3H), 1.22 (s, 3H), 1.01 (s, 3H). | LCMS (ES, m/z): 566 [M + H]+, Rt 0.875 min. |
| 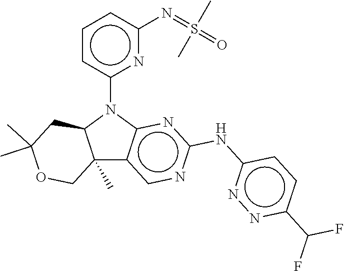 | 377 | 1H NMR (400 MHz, DMSO-d6) δ 10.75 (s, 1H), 8.74 (d, J = 9.6 Hz, 1H), 8.22 (d, J = 8.0 Hz, 1H), 8.14 (s, 1H), 7.96 (d, J = 9.6 Hz, 1H), 7.58 (t, J = 8.0 Hz, 1H), 7.35-7.02 (m, 1H), 6.36 (d, J = 7.8 Hz, 1H), 4.68-4.55 (m, 1H), 3.87-3.82 (m, 1H), 3.72-3.66 (m, 1H), 3.52 (s, 3H), 3.31 (s, 3H), 2.50-2.42 (m, 1H), 1.62-1.50 (m, 1H), 1.30 (s, 3H), 1.23 (s, 3H), 1.01 (s, 3H). | LCMS (ES, m/z): 531 [M + H]+, Rt 0.666 min. |
| 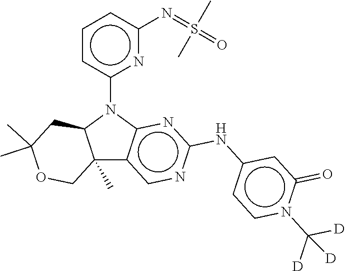 | 378 | 1H NMR (400 MHz, DMSO-d6) δ 9.54 (s, 1H), 8.11 (s, 1H), 8.06 (d, J = 8.0 Hz, 1H), 7.56 (t, J = 8.0 Hz, 1H), 7.50 (d, J = 7.2 Hz, 1H), 7.09 (s, 1H), 6.51 (d, J = 7.6 Hz, 1H), 6.36 (d, J = 8.0 Hz, 1H), 4.58-4.54 (m, 1H), 3.87-3.84 (m, 1H), 3.68-3.65 (m, 1H), 3.49 (s, 3H), 3.30 (s, 3H), 2.47-2.42 (m, 1H), 1.57-1.51 (m, 1H), 1.28 (s, 3H), 1.22 (s, 3H), 1.01 (s, 3H). | LCMS (ES, m/z): 513 [M + H]+, Rt 1.118 min. |
| 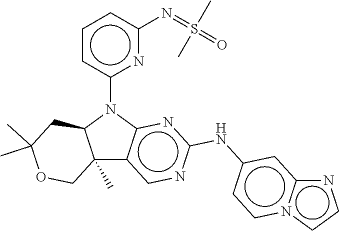 | 379 | 1H NMR (400 MHz, DMSO-d6) δ 9.55 (s, 1H), 8.30 (d, J = 6.8 Hz, 1H), 8.20 (s, 1H), 8.07 (s, 1H), 8.04 (s, 1H), 7.66 (s, 1H), 7.52 (t, J = 8.0 Hz, 1H), 7.33 (s, 1H), 7.05- 7.01 (m, 1H), 6.29 (d, J = 7.6 Hz, 1H), 4.54-4.45 (m, 1H), 3.87-3.82 (m, 1H), 3.66-3.61 (m, 1H), 3.43 (s, 3H), 3.24 (s, 3H), 2.44-2.35 (m, 1H), 1.53-1.43 (m, 1H), 1.23 (s, 3H), 1.16 (s, 3H), 0.95 (s, 3H). | LCMS (ES, m/z): 519 [M + H]+; RT: 0.551 min. |
| 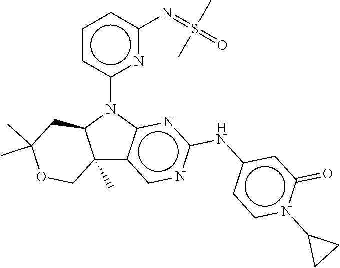 | 380 | 1H NMR (400 MHz, DMSO-d6) δ 9.54 (s, 1H), 8.11 (s, 1H), 8.05 (d, J = 8.0 Hz, 1H), 7.55 (t, J = 8.0 Hz, 1H), 7.34 (d, J = 7.6 Hz, 1H), 7.08 (s, 1H), 6.55-6.46 (m, 1H), 6.35 (d, J = 7.6 Hz, 1H), 4.70-4.45 (m, 1H), 4.00-3.78 (m, 1H), 3.78-3.62 (m, 1H), 3.49 (s, 3H), 3.30 (s, 3H), 3.28-3.15 (m, 1H), 2.49-2.39 (m, 1H), 1.72-1.45(m, 1H), 1.28 (s, 3H), 1.22 (s, 3H), 1.01 (s, 3H), 0.99-0.88 (m, 2H), 0.88-0.66 (m, 2H). | LCMS (ES, m/z): 536 [M + H]+, Rt 0.608 min. |
| 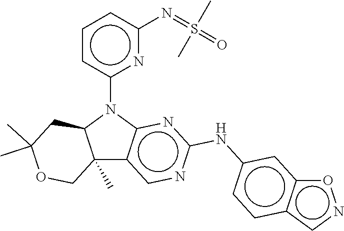 | 381 | 1H NMR (300 MHz, DMSO-d6) δ 10.77 (br, 1H), 9.62 (s, 1H), 8.08- 7.97 (m, 2H), 7.59-7.47 (m, 2H), 7.36 (d, J = 8.4 Hz, 1H), 7.27-7.18 (m, 1H), 6.27 (d, J = 7.8 Hz, 1H), 4.54-4.43 (m, 1H), 3.83-3.72 (m, 1H), 3.68-3.56 (m, 1H), 3.42 (s, 3H), 3.23 (s, 3H), 2.43-2.32 (m, 1H), 1.52-1.38 (m, 1H), 1.19 (s, 3H), 1.15 (s, 3H), 0.95 (s, 3H). | LCMS (ES, m/z): 520 [M + H]+; RT: 1.231 min. |
| 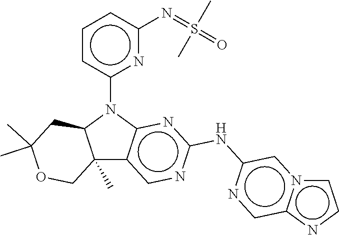 | 382 | 1H NMR (300 MHz, DMSO-d6) δ 9.54 (s, 1H), 9.27 (s, 1H), 8.85 (s, 1H), 8.14 (d, J = 8.1 Hz, 1H), 8.09 (s, 1H), 8.03 (s, 1H), 7.70 (s, 1H), 7.48 (t, J = 8.4 Hz, 1H), 6.28 (d, J = 7.8 Hz, 1H), 4.54-4.44 (m, 1H), 3.83-3.72 (m, 1H), 3.68-3.56 (m, 1H), 3.42 (s, 3H), 3.23 (s, 3H), 2.45-2.37 (m, 1H), 1.53-1.40 (m, 1H), 1.19 (s, 3H), 1.15 (s, 3H), 0.95 (s, 3H). | LCMS (ES, m/z): 520 [M + H]+; RT: 1.099 min. |
| 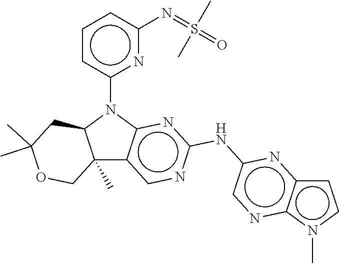 | 383 | 1H NMR (400 MHz, DMSO-d6) δ 9.63 (s, 1H), 8.99 (s, 1H), 8.21 (d, J = 8.0 Hz, 1H), 8.06 (s, 1H), 7.77 (s, 1H), 7.49 (t, J = 8.0 Hz, 1H), 6.47 (d, J = 3.6 Hz, 1H), 6.32 (d, J = 7.6 Hz, 1H), 4.57-4.53 (m, 1H), 3.90-3.85 (m, 1H), 3.84 (s, 3H), 3.67-3.61 (m, 1H), 3.49 (s, 3H), 3.29 (s, 3H), 2.50-2.42 (m, 1H), 1.57-1.47 (m, 1H), 1.29 (s, 3H), 1.23 (s, 3H), 1.02 (s, 3H). | LCMS (ES, m/z): 534 [M + H]+, Rt 0.688 min. |
| 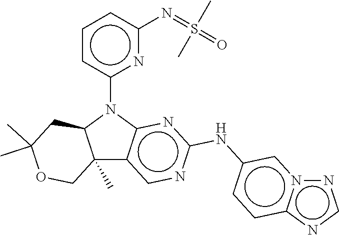 | 384 | 1H NMR (400 MHz, DMSO-d6) δ 9.75-9.71 (m, 2H), 8.39 (s, 1H), 8.15 (s, 1H), 8.07 (s, 1H), 7.80 (s, 2H), 7.60 (t, J = 8.0 Hz, 1H), 6.37 (d, J = 7.6 Hz, 1H), 4.59-4.55 (m, 1H), 3.87-3.82 (m, 1H), 3.66-3.61 (m, 1H), 3.50 (s, 3H), 3.31 (s, 3H), 2.50-2.44 (m, 1H), 1.59-1.53 (m, 1H), 1.30 (s, 3H), 1.23 (s, 3H), 1.02 (s, 3H). | LCMS (ES, m/z): 520 [M + H]+, Rt 0.579 min. |
| 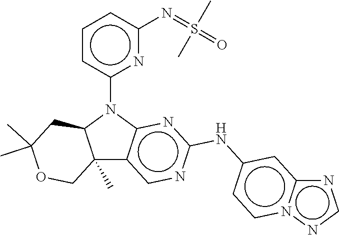 | 385 | 1H NMR (400 MHz, DMSO-d6) δ 9.98 (s, 1H), 8.76 (d, J = 7.6 Hz, 1H), 8.52 (s, 1H), 8.32-7.92 (m, 3H), 7.70-7.04 (m, 2H), 6.39 (d, J = 7.6 Hz, 1H), 4.59-4.53 (m, 1H), 3.87-3.82 (m, 1H), 3.66-3.61 (m, 1H), 3.50 (s, 3H), 3.32 (s, 3H), 2.50-2.46 (m, 1H), 1.61-1.55 (m, 1H), 1.31 (s, 3H), 1.24 (s, 3H), 1.03 (s, 3H). | LCMS (ES, m/z): 520 [M + H]+, Rt 1.197 min. |
| 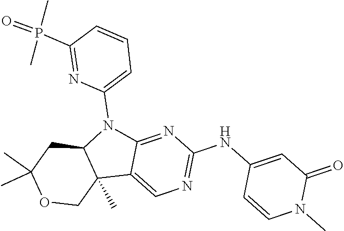 [SMILES: Cn1ccc(Nc2ncc3c(n2)N(c2cccc(P(C)(C)=O)n2)[C@@H]2CC(C)(C)OC[C@]32C)cc1=O] | 779 | 1H NMR (400 MHz, DMSO-d6) δ 9.66 (s, 1H), 8.79-8.77 (m, 1H), 8.18 (s, 1H), 8.01-7.96 (m, 1H), 7.61 (t, J = 5.6 Hz, 1H), 7.53 (d, J = 7.6 Hz, 1H), 7.09 (d, J = 2.4 Hz, 1H), 6.50-6.48 (m, 1H), 4.69- 4.66 (m, 1H), 3.83-3.68 (m, 2H), 3.33 (s, 3H), 2.23-2.18 (m, 1H), 1.71-1.59 (m, 7H), 1.32 (s, 3H), 1.24 (s, 3H), 1.04 (s, 3H). | LCMS (ES, m/z): 495.15 [M + H]+, Rt 0.550 min. |
| 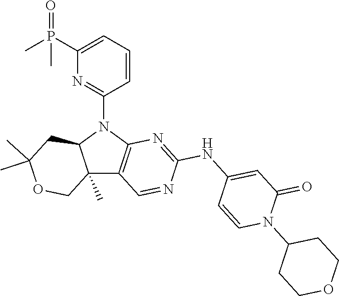 [SMILES: CC1(C)C[C@H]2N(c3cccc(P(C)(C)=O)n3)c3nc(Nc4ccn(C5CCOCC5)c(=O)c4)ncc3[C@@]2(C)CO1] | 780 | 1H NMR (400 MHz, DMSO-d6) δ 9.68 (s, 1H), 8.78 (d, J = 8.8 Hz, 1H), 8.18 (s, 1H), 8.05-7.96 (m, 1H), 7.64-7.56 (m, 2H), 7.10 (d, J = 2.4 Hz, 1H), 6.61-6.52 (m, 1H), 4.93-4.80 (m, 1H), 4.72-4.62 (m, 1H), 4.03-3.90 (m, 2H), 3.87- 3.77 (m, 1H), 3.74-3.63 (m, 1H), 3.45 (t, J = 11.2 Hz, 2H), 2.27- 2.18 (m, 1H), 1.92-1.76 (m, 2H), 1.75-1.55 (m, 9H), 1.33 (s, 3H), 1.24 (s, 3H), 1.04 (s, 3H). | LCMS (ES, m/z): 565.25 [M + H]+, Rt 0.600 min. |
| 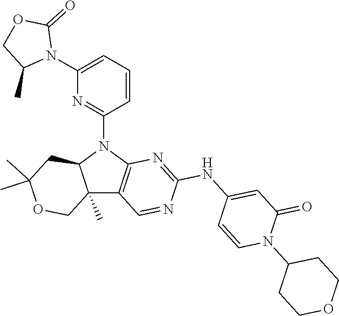 [SMILES: C[C@H]1COC(=O)N1c1cccc(N2c3nc(Nc4ccn(C5CCOCC5)c(=O)c4)ncc3[C@@]3(C)COC(C)(C)C[C@@H]23)n1] | 781 | 1H NMR (300 MHz, DMSO-d6) δ 9.63 (s, 1H), 8.36 (d, J = 8.1 Hz, 1H), 8.16 (s, 1H), 7.85 (t, J = 8.1 Hz, 1H), 7.68 (d, J = 7.8 Hz, 1H), 7.58 (d, J = 7.8 Hz, 1H), 7.09 (d, J = 2.4 Hz, 1H), 6.57-6.54 (m, 1H), 4.91-4.80 (m, 2H), 4.75-4.71 (m, 1H), 4.54 (t, J = 8.4 Hz, 1H), 4.14-4.10 (m, 1H), 3.99-3.94 (m, 2H), 3.89-3.85 (m, 1H), 3.71-3.67 (m, 1H), 3.67-3.41 (m, 2H), 2.15- 2.03 (m, 1H), 1.86-1.81 (m, 2H), 1.66-1.61 (m, 3H), 1.38 (d, J = 6.0 Hz, 3H), 1.27(s, 3H), 1.23 (s, 3H), 1.05 (s, 3H). | LCMS (ES, m/z): 588.25 [M + H]+, Rt 0.742 min. |
| 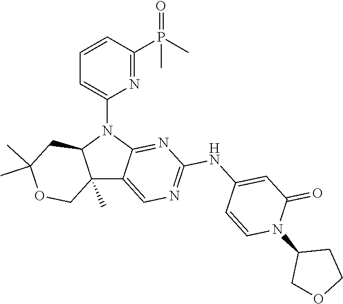 [SMILES: CC1(C)C[C@H]2N(c3cccc(P(C)(C)=O)n3)c3nc(Nc4ccn([C@H]5CCOC5)c(=O)c4)ncc3[C@@]2(C)CO1] | 782, 783 | 1H NMR (400 MHz, DMSO-d6) δ 9.70 (s, 1H), 8.79 (d, J = 8.4 Hz, 1H), 8.19 (s, 1H), 8.04-7.95 (m, 1H), 7.64-7.58 (m, 1H), 7.44 (d, J = 7.6 Hz, 1H), 7.12 (d, J = 2.4 Hz, 1H), 6.58-6.51 (m, 1H), 5.38- 5.28 (m, 1H), 4.68 (dd, J = 9.2, 4.8 Hz, 1H), 4.04 (q, J = 8.0 Hz, 1H), 3.88-3.65 (m, 5H), 2.45-2.30 (m, 1H), 2.26-2.15 (m, 1H), 2.00- 1.86 (m, 1H), 1.78-1.54 (m, 7H), 1.32 (s, 3H), 1.24 (s, 3H), 1.04 (s, 3H). | LCMS (ES, m/z): 551.25 [M + H]+, Rt 0.583 min. |
| 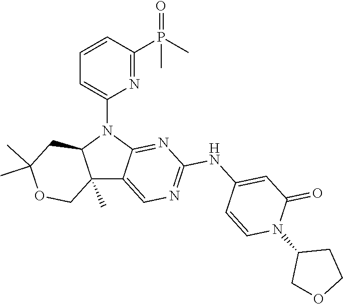 [SMILES: CC1(C)C[C@H]2N(c3cccc(P(C)(C)=O)n3)c3nc(Nc4ccn([C@@H]5CCOC5)c(=O)c4)ncc3[C@@]2(C)CO1] | | 1H NMR (400 MHz, DMSO-d6) δ 9.70 (s, 1H), 8.80 (d, J = 9.6 Hz, 1H), 8.19 (s, 1H), 8.01-7.96 (m, 1H), 7.61 (t, J = 5.6 Hz, 1H), 7.45 (d, J = 7.6 Hz, 1H), 7.13 (d, J = 2.4 Hz, 1H), 6.56 (m, 1H), 5.36- 5.31 (m, 1H), 4.69-4.66 (m, 1H), 4.07-4.01 (m, 1H), 3.82-3.68 (m, 5H), 2.43-2.34 (m, 1H), 2.23-2.19 (m, 1H), 1.97-1.89 (m, 1H), 1.71- 1.59 (m, 7H), 1.33 (s, 3H), 1.24 (s, 3H) , 1.04 (s, 3H). | LCMS (ES, m/z): 551.25 [M + H]+, Rt 0.583 min. |
| 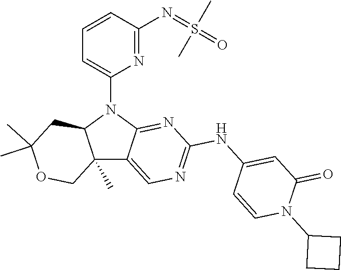 [SMILES: CC1(C)C[C@H]2N(c3cccc(N=S(C)(C)=O)n3)c3nc(Nc4ccn(C5CCC5)c(=O)c4)ncc3[C@@]2(C)CO1] | 802 | 1H NMR (400 MHz, DMSO-d6) δ 9.56 (s, 1H), 8.11 (s, 1H), 8.05 (d, J = 8.0 Hz, 1H), 7.66 (d, J = 7.6 Hz, 1H), 7.57 (t, J = 8.0 Hz, 1H), 7.05 (s, 1H), 6.70-6.45 (m, 1H), 6.35 (d, J = 7.6 Hz, 1H), 5.19-4.90 (m, 1H), 4.69-4.39 (m, 1H), 3.88- 3.80 (m, 1H), 3.75-3.65 (m, 1H), 3.49 (s, 3H), 3.31 (s, 3H), 2.45- 2.41(m, 1H), 2.29-2.09 (m, 4H), 1.89-1.61(m, 2H), 1.61-1.39 (m, 1H), 1.28 (s, 3H), 1.22 (s, 3H), 1.01 (s, 3H). | LCMS (ES, m/z): 550.30 [M + H]+, Rt 0.647 min. |
| 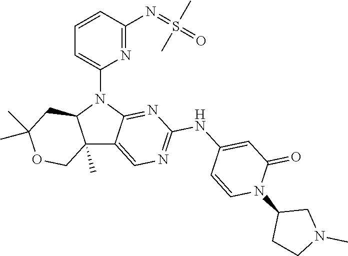 | 804, 805 | 1H NMR (400 MHz, DMSO-d6) δ 9.57 (s, 1H), 8.11 (s, 1H), 8.06 (d, J = 8.0 Hz, 1H), 7.67 (d, J = 7.6 Hz, 1H), 7.56 (t, J = 8.0 Hz, 1H), 7.11 (s, 1H), 6.61-6.41 (m, 1H), 6.35 (d, J = 7.6 Hz, 1H), 5.40-5.19 (m, 1H), 4.66-4.49 (m, 1H), 3.89- 3.78 (m, 1H), 3.78-3.60 (m, 1H), 3.49 (s, 3H), 3.31 (s, 3H), 3.05- 2.95 (m, 1H), 2.78-2.68 (m, 1H), 2.48-2.40 (m, 2H), 2.39-2.25 (m, 4H), 2.25-2.15 (m, 1H), 1.78-1.61 (m, 1H), 1.61-1.45 (m, 1H), 1.28 (s, 3H), 1.22 (s, 3H), 1.01 (s, 3H). | LCMS (ES, m/z): 579.30 [M + H]+, Rt 1.123 min. |
| 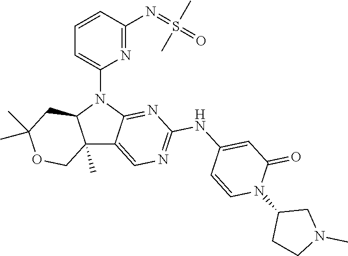 | | 1H NMR (400 MHz, DMSO-d6) δ 9.55 (s, 1H), 8.11 (s, 1H), 8.07 (d, J = 8.0 Hz, 1H), 7.67 (d, J = 7.6 Hz, 1H), 7.56 (t, J = 8.0 Hz, 1H), 7.10 (d, J = 2.4 Hz, 1H), 6.57- 6.50 (m, 1H), 6.35 (d, J = 7.8 Hz, 1H), 5.37-5.26 (m, 1H), 4.61-4.53 (m, 1H), 3.87-3.81 (m, 1H), 3.68- 3.63 (m, 1H), 3.48 (s, 3H), 3.29 (s, 3H), 3.00-2.93 (m, 1H), 2.74-2.68 (m, 1H), 2.48-2.43 (m, 2H), 2.38- 2.32 (m, 1H), 2.28 (s, 3H), 2.24- 2.14 (m, 1H), 1.72-1.63 (m, 1H), 1.59-1.49 (m, 1H), 1.28 (s, 3H), 1.22 (s, 3H), 1.00 (s, 3H). | LCMS (ES, m/z): 579.20 [M + H]+, Rt 0.848 min. |
| 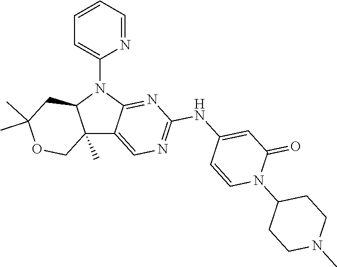 | 806 | 1H NMR (300 MHz, DMSO-d6) δ 9.59 (s, 1H), 8.57 (d, J = 8.4 Hz, 1H), 8.40 (d, J = 3.6 Hz, 1H), 8.15 (s, 1H), 7.83 (t, J = 6.9 Hz, 1H), 7.55 (d, J = 7.5 Hz, 1H), 7.20-6.95 (m, 2H), 6.65-6.40 (m, 1H), 4.79- 4.68 (m, 1H), 4.68-4.47 (m, 1H), 3.99-3.79 (m, 1H), 3.75-3.55 (m, 1H), 3.01-2.75 (m, 2H), 2.20-2.08 (m, 4H), 2.07-1.91 (m, 2H), 1.91- 1.72 (m, 2H), 1.72-1.49 (m, 3H), 1.23 (s, 3H), 1.19 (s, 3H), 0.99 (s, 3H). | LCMS (ES, m/z): 502.30 [M + H]+, Rt 0.963 min. |
| 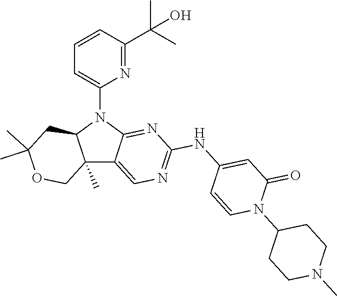 | 810 | 1H NMR (300 MHz, DMSO-d6) δ 9.59 (s, 1H), 8.44 (d, J = 8.1 Hz, 1H), 8.11 (s, 1H), 7.90-7.70 (m, 1H), 7.55 (d, J = 7.8 Hz, 1H), 7.33 (d, J = 6.9 Hz, 1H), 7.10 (s, 1H), 6.65-6.41 (m, 1H), 5.20 (br, 1H), 4.78-4.41 (m, 2H), 3.99-3.75 (m, 1H), 3.74-3.62 (m, 1H), 3.02-2.71 (m, 2H), 2.29-2.22 (m, 1H), 2.20 (s, 3H), 2.11-1.91 (m, 2H), 1.90- 1.72 (m, 2H), 1.71-1.53 (m, 3H), 1.46 (s, 6H), 1.31 (s, 3H), 1.23 (s, 3H), 1.03 (s, 3H). | LCMS (ES, m/z): 560.30 [M + H]+, Rt 0.600 min. |
| 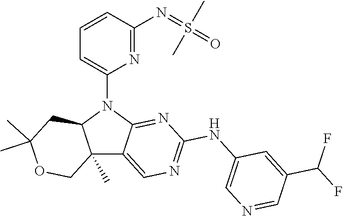 [SMILES: CC1(C)C[C@H]2N(c3cccc(N=S(C)(C)=O)n3)c3nc(Nc4cncc(C(F)F)c4)ncc3[C@@]2(C)CO1] | 815 | 1H NMR (300 MHz, DMSO-d6) δ 9.82 (s, 1H), 8.93 (s, 1H), 8.60 (s, 1H), 8.31 (s, 1H), 8.12 (s, 1H), 8.00 (d, J = 8.1 Hz, 1H), 7.55 (t, J = 7.8 Hz, 1H), 7.37-6.92 (m, 1H), 6.35 (d, J = 7.8 Hz, 1H), 4.65- 4.53 (m, 1H), 3.91-3.81 (m, 1H), 3.73-3.60 (m, 1H), 3.49 (s, 3H), 3.30 (s, 3H), 2.49-2.40 (m, 1H), 1.63-1.45 (m, 1H), 1.30 (s, 3H), 1.22 (s, 3H), 1.02 (s, 3H). | LCMS (ES, m/z): 530.15 [M + H]+, Rt 0.640 min. |
| 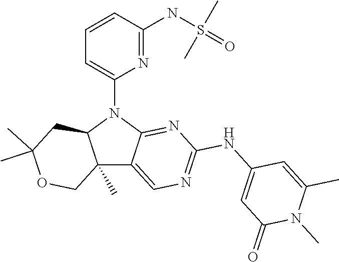 | 816 | 1H NMR (400 MHz, DMSO-d6) δ 9.46 (s, 1H), 8.10 (s, 1H), 8.04 (d, J = 8.0 Hz, 1H), 7.55 (t, J = 8.0 Hz, 1H), 6.91 (d, J = 2.0 Hz, 1H), 6.47 (d, J = 2.0 Hz, 1H), 6.36 (d, J = 7.6 Hz, 1H), 4.60-4.50 (m, 1H), 3.87-3.81 (m, 1H), 3.68-3.63 (m, 1H), 3.49 (s, 3H), 3.35-3.25 (m, 6H), 2.48-2.41 (m, 1H), 2.29 (s, 3H), 1.58-1.50 (m, 1H), 1.27 (s, 3H), 1.22 (s, 3H), 1.00 (s, 3H). | LCMS (ES, m/z): 524.15 [M + H]+, Rt 0.597 min. |
| 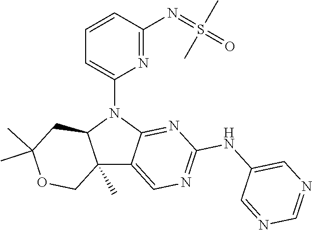 [SMILES: CC1(C)C[C@H]2N(c3cccc(N=S(C)(C)=O)n3)c3nc(Nc4cncnc4)ncc3[C@@]2(C)CO1] | 817 | 1H NMR (400 MHz, DMSO-d6) δ 9.69 (s, 1H), 9.18 (s, 2H), 8.74 (s, 1H), 8.10 (s, 1H), 8.02 (d, J = 8.0 Hz, 1H), 7.56 (t, J = 8.0 Hz, 1H), 6.35 (d, J = 8.0 Hz, 1H), 4.66-4.48 (m, 1H), 3.90-3.79 (m, 1H), 3.72- 3.62 (m, 1H), 3.30 (s, 3H), 3.33- 3.32 (m, 1H), 3.31-3.30 (m, 2H), 2.48-2.43 (m, 1H), 1.60-1.50 (m, 1H), 1.30 (s, 3H), 1.23 (s, 3H), 1.01 (s, 3H). | LCMS (ES, m/z): 481.10 [M + H]+, Rt 0.988 min. |
| 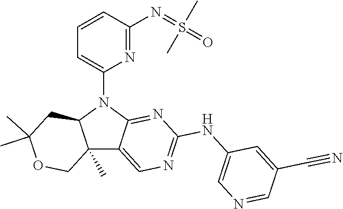 [SMILES: CC1(C)C[C@H]2N(c3cccc(N=S(C)(C)=O)n3)c3nc(Nc4cncc(C#N)c4)ncc3[C@@]2(C)CO1] | 818 | 1H NMR (400 MHz, DMSO-d6) δ 9.97 (s, 1H), 9.00 (d, J = 2.8 Hz, 1H), 8.83 (s, 1H), 8.54 (s, 1H), 8.13 (s, 1H), 7.95 (d, J = 8.0 Hz, 1H), 7.59 (t, J = 8.0 Hz, 1H), 6.43- 6.34 (m, 1H), 4.63-4.53 (m, 1H), 3.88-3.81 (m, 1H), 3.72-3.65 (m, 1H), 3.48 (s, 3H), 3.30 (s, 3H), 2.47-2.40 (m, 1H), 1.62-1.50 (m, 1H), 1.28 (s, 3H), 1.22 (s, 3H), 1.01 (s, 3H). | LCMS (ES, m/z): 505.20 [M + H]+, Rt 0.642 min. |
| 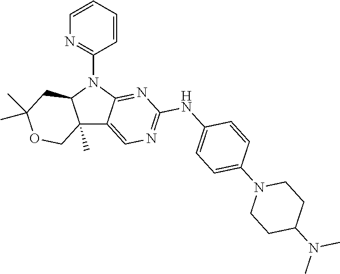 | 819 | 1H NMR (300 MHz, DMSO-d6) δ 9.08 (s, 1H), 8.63 (d, J = 8.7 Hz, 1H), 8.37 (d, J = 0.9 Hz, 1H), 8.01 (s, 1H), 7.81 (t, J = 1.8 Hz, 1H), 7.52 (d, J = 9.0 Hz, 2H), 7.02 (d, J = 5.1 Hz, 1H), 6.90 (d, J = 9.0 Hz, 2H), 4.83-4.51 (m, 1H), 3.85- 3.74 (m, 1H), 3.70-3.55 (m, 3H), 2.66-2.53 (m, 2H), 2.25-2.07 (m, 8H), 1.90-1.79 (m, 2H), 1.68-1.40 (m, 3H), 1.24 (s, 3H), 1.21 (s, 3H), 1.00 (s, 3H). | LCMS (ES, m/z): 514.25 [M + H]+, Rt 0.595 min. |
| 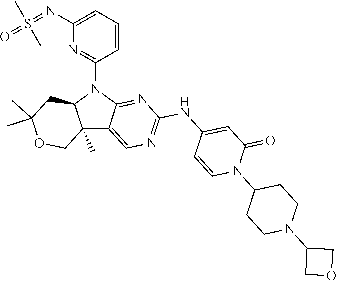 [SMILES: CC1(C)C[C@H]2N(c3cccc(N=S(C)(C)=O)n3)c3nc(Nc4ccn(C5CCN(C6COC6)CC5)c(=O)c4)ncc3[C@@]2(C)CO1] | 824 | 1H NMR (300 MHz, DMSO-d6) δ 9.55 (s, 1H), 8.10 (s, 1H), 8.03 (d, J = 8.1 Hz, 1H), 7.64-7.53 (m, 2H), 7.08 (d, J = 2.1 Hz, 1H), 6.61-6.49 (m, 1H), 6.35 (d, J = 7.8 Hz, 1H), 4.74-4.35 (m, 6H), 3.89- 3.81 (m, 1H), 3.68-3.60 (m, 1H), 3.51-3.39 (m, 4H), 3.30 (s, 3H), 2.88-3.81 (m, 2H), 2.49-2.38 (m, 1H), 2.03-1.75 (m, 4H), 1.73-1.63 (m, 2H), 1.61-1.47 (m, 1H), 1.28 (s, 3H), 1.22 (s, 3H), 1.01 (s, 3H). | LCMS (ES, m/z): 635.40 [M + H]+, Rt 1.070 min. |
| 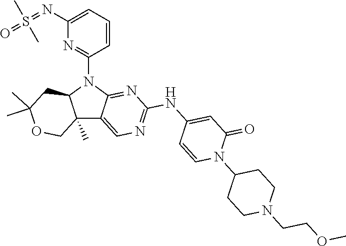 [SMILES: COCCN1CCC(n2ccc(Nc3ncc4c(n3)N(c3cccc(N=S(C)(C)=O)n3)[C@@H]3CC(C)(C)OC[C@]43C)cc2=O)CC1] | 825 | 1H NMR (400 MHz, DMSO-d6) δ 9.56 (s, 1H), 8.11 (s, 1H), 8.04 (d, J = 8.0 Hz, 1H), 7.58 (t, J = 7.6 Hz, 2H), 7.09 (d, J = 2.4 Hz, 1H), 6.58-6.51 (m, 1H), 6.35 (d, J = 7.6 Hz, 1H), 4.66-4.52 (m, 2H), 3.89-3.81 (m, 1H), 3.70-3.62 (m, 1H), 3.49 (s, 3H), 3.44 (t, J = 5.6 Hz, 2H), 3.34 (s, 4H), 3.24 (s, 3H), 3.03-2.96 (m, 2H), 2.51-2.40 (m, 2H), 2.14-2.04 (m, 2H), 1.86-1.72 (m, 2H), 1.68-1.59 (m, 2H), 1.59- 1.49 (m, 1H), 1.28 (s, 3H), 1.22 (s, 3H), 1.01 (s, 3H). | LCMS (ES, m/z): 637.40 [M + H]+, Rt 1.148 min. |

### Intermediate 304. tert-butyl N-[[6-[(1R,9R)-5-amino-1,11,11-trimethyl-12-oxa-4,6,8-triazatricyclo[7.4.0.02,7]trideca-2(7),3,5-trien-8-yl]-2-pyridyl]methyl]-N-methyl-carbamate

### Step 1. tert-butyl N-methyl-N-[[6-[(1R,9R)-1,11,11-trimethyl-5-methylsulfanyl-12-oxa-4,6,8-triazatricyclo[7.4.0.02,7]trideca-2,4,6-trien-8-yl]-2-pyridyl]methyl]carbamate

To a stirred solution of Intermediate 300 (2 g, 7.54 mmol, 1 eq.) and Intermediate 2 (1.97 g, 8.29 mmol, 1.1 eq.) in 1,4-dioxane (18 mL) were added Pd₂dba₃ (689.59 mg, 753.65 μmol, 0.1 eq.), BINAP (938.56 mg, 1.51 mmol, 0.2 eq) and cesium carbonate (4.91 g, 15.07 mmol, 2 eq.) in portions. The resulting mixture was stirred for 2 h at 100° C. under N₂ atmosphere. The mixture was allowed to cool down to room temperature. The resulting mixture was filtered, the filter cake was washed with DCM (3×10 mL). The filtrate was concentrated under reduced pressure. The residue was purified by silica gel column chromatography, eluted with [EA/PE](1:3) to afford tert-butyl N-methyl-N-[[6-[(1R,9R)-1,11,11-trimethyl-5-methylsulfanyl-12-oxa-4,6,8-triazatricyclo[7.4.0.02,7]trideca-2,4,6-trien-8-yl]-2-pyridyl]methyl]carbamate (2.83 g, 5.77 mmol, 77% yield) as a yellow oil.

### Step 2. tert-butyl N-methyl-N-[[6-[(1R,9R)-1,11,11-trimethyl-5-methylsulfinyl-12-oxa-4,6,8-triazatricyclo[7.4.0.02,7]trideca-2(7),3,5-trien-8-yl]-2-pyridyl]methyl]carbamate

To a stirred solution of tert-butyl N-methyl-N-[[6-[(1R,9R)-1,11,11-trimethyl-5-methylsulfanyl-12-oxa-4,6,8-triazatricyclo[7.4.0.02,7]trideca-2(7),3,5-trien-8-yl]-2-pyridyl]methyl]carbamate (1 g, 2.06 mmol, 1 eq.) in DCM (10 mL) was added m-CPBA (426 mg, 2.47 mmol, 1.2 eq.) dropwise at 0° C. The resulting mixture was stirred for 1 h at room temperature. The reaction was monitored by TLC. The resulting mixture was concentrated under reduced pressure. The residue was purified by silica gel column chromatography (eluting with 10:1 DCM/MeOH) to afford tert-butyl N-methyl-N-[[6-[(1R,9R)-1,11,11-trimethyl-5-methylsulfinyl-12-oxa-4,6,8-triazatricyclo[7.4.0.02,7]trideca-2(7),3,5-trien-8-yl]-2-pyridyl]methyl]carbamate (950 mg, 1.90 mmol, 91% yield) as a light-yellow oil. LCMS (ES, m/z):502 [M+H]+, Rt 0.692 min.

### Step 3. tert-butyl N-[[6-[(1R,9R)-5-amino-1,11,11-trimethyl-12-oxa-4,6,8-triazatricyclo[7.4.0.02,7]trideca-2(7),3,5-trien-8-yl]-2-pyridyl]methyl]-N-methyl-carbamate (Intermediate 304)

Into a mixture of tert-butyl N-methyl-N-[[6-[(1R,9R)-1,11,11-trimethyl-5-methylsulfinyl-12-oxa-4,6,8-triazatricyclo[7.4.0.02,7]trideca-2(7),3,5-trien-8-yl]-2-pyridyl]methyl]carbamate (900 mg, 1.79 mmol, 1 eq.) in dioxane (4 mL) was added ammonia (25%, 12 mL) slowly. The mixture was stirred for 16 h at 80° C. The reaction was monitored by LCMS. The mixture was allowed to cool down to room temperature. The resulting mixture was concentrated under reduced pressure. The residue was purified by silica gel column chromatography (eluting with 10:1 DCM/MeOH) to afford tert-butyl N-[[6-[(1R,9R)-5-amino-1,11,11-trimethyl-12-oxa-4,6,8-triazatricyclo[7.4.0.02,7]trideca-2(7),3,5-trien-8-yl]-2-pyridyl]methyl]-N-methyl-carbamate (390 mg, 0.86 mmol, 48% yield) as a light yellow oil. LCMS (ES, m/z):455 [M+H]+, Rt 0.533 min.

### Example 386: 1-tetrahydropyran-4-yl-4-[[(1R,9R)-1,11,11-trimethyl-8-[6-(methylaminomethyl)-2-pyridyl]-12-oxa-4,6,8-triazatricyclo[7.4.0.02,7]trideca-2(7),3,5-trien-5-yl]amino]pyridin-2-one

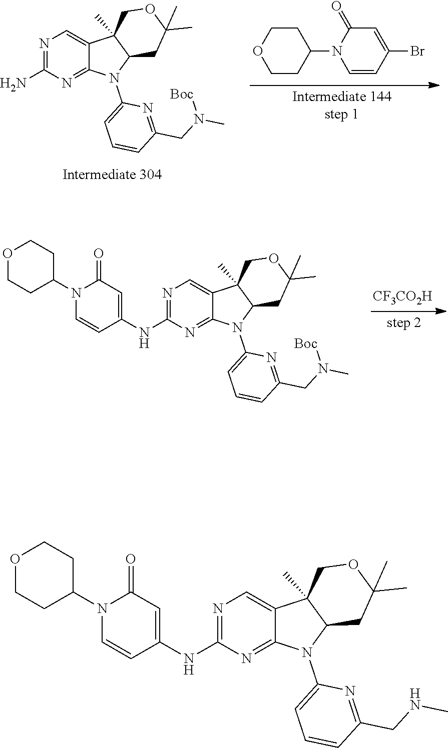 [SMILES: CN(Cc1cccc(N2c3nc(N)ncc3[C@@]3(C)COC(C)(C)C[C@@H]23)n1)C(=O)OC(C)(C)C ; O=c1cc(Br)ccn1C1CCOCC1 ; CN(Cc1cccc(N2c3nc(Nc4ccn(C5CCOCC5)c(=O)c4)ncc3[C@@]3(C)COC(C)(C)C[C@@H]23)n1)C(=O)OC(C)(C)C ; "CF3CO2H" ; CNCc1cccc(N2c3nc(Nc4ccn(C5CCOCC5)c(=O)c4)ncc3[C@@]3(C)COC(C)(C)C[C@@H]23)n1]

### Step 1. tert-butyl N-methyl-N-[[6-[(1R,9R)-1,11,11-trimethyl-5-[(2-oxo-1-tetrahydropyran-4-yl-4-pyridyl)amino]-12-oxa-4,6,8-triazatricyclo[7.4.0.02,7]trideca-2(7),3,5-trien-8-yl]-2-pyridyl]methyl]carbamate

To a stirred mixture of tert-butyl N-[[6-[(1R,9R)-5-amino-1,11,11-trimethyl-12-oxa-4,6,8-triazatricyclo[7.4.0.02,7]trideca-2(7),3,5-trien-8-yl]-2-pyridyl]methyl]-N-methyl-carbamate (Intermediate 304, 50 mg, 110 μmol, 1 eq.) and 4-bromo-1-tetrahydropyran-4-yl-pyridin-2-one (Intermediate 144, 34.07 mg, 132.0 μmol, 1.2 eq.) in dioxane (2 mL) were added Xphos (10.47 mg, 22.00 μmol, 0.2 eq.), XPhos Pd G3 (9.31 mg, 11.00 μmol, 0.1 eq.) and Cs₂CO₃ (107.52 mg, 329.99 μmol, 3.0 eq.). The resulting mixture was stirred for 2 h at 100° C. under N2 atmosphere. The reaction was monitored by LCMS. The mixture was allowed to cool down to room temperature. The solids were filtered out and washed with EA (3×5 mL). The filtrate was concentrated under reduced pressure. The residue was purified by prep-TLC (eluting with 1:1 EA/PE) to afford tert-butyl N-methyl-N-[[6-[(1R,9R)-1,11,11-trimethyl-5-[(2-oxo-1-tetrahydropyran-4-yl-4-pyridyl)amino]-12-oxa-4,6,8-triazatricyclo[7.4.0.02,7]trideca-2(7),3,5-trien-8-yl]-2-pyridyl]methyl]carbamate (50 mg, 79.23 μmol, 72% yield) as a light-yellow solid. LCMS (ES, m/z):632 [M+H]⁺, Rt 1.053 min.

### Step 2. 1-tetrahydropyran-4-yl-4-[[(1R,9R)-1,11,11-trimethyl-8-[6-(methylaminomethyl)-2-pyridyl]-12-oxa-4,6,8-triazatricyclo[7.4.0.02,7]trideca-2(7),3,5-trien-5-yl]amino]pyridin-2-one (Example 386)

To a solution of tert-butyl N-methyl-N-[[6-[(1R,9R)-1,11,11-trimethyl-5-[(2-oxo-1-tetrahydropyran-4-yl-4-pyridyl)amino]-12-oxa-4,6,8-triazatricyclo[7.4.0.02,7]trideca-2(7),3,5-trien-8-yl]-2-pyridyl]methyl]carbamate (40 mg, 63.31 μmol, 1 eq.) in DCM (3 mL) was added TFA (2 mL) dropwise at 0° C. The resulting mixture was stirred for 1 h at room temperature. The reaction was monitored by LCMS. The resulting mixture was concentrated under reduced pressure. The residue was purified by Prep-HPLC (Column: XBridge Prep OBD C18 Column, 30*150 mm, 5 μm; Mobile Phase A: Water(10 mmol/L NH₄HCO₃), Mobile Phase B: ACN; Flow rate: 60 mL/min; Gradient: 17% B to 39% B in 7 min, 39% B; Wave Length: 254 nm) to afford 1-tetrahydropyran-4-yl-4-[[(1R,9R)-1,11,11-trimethyl-8-[6-(methylaminomethyl)-2-pyridyl]-12-oxa-4,6,8-triazatricyclo[7.4.0.02,7]trideca-2(7),3,5-trien-5-yl]amino]pyridin-2-one (24.1 mg, 45.0 μmol, 71% yield) as a white solid. ¹H NMR (400 MHz, DMSO-d6) δ 9.59 (s, 1H), 8.43 (d, J=8.4 Hz, 1H), 8.13 (s, 1H), 7.78 (t, J=8.0 Hz, 1H), 7.58 (d, J=7.6 z, 1H), 7.10 (s, 1H), 7.08 (d, J=7.6 z, 1H), 6.68-6.43 (m, 1H), 4.99-4.80 (m, 1H), 4.79-4.55 (m, 1H), 4.12-3.90 (m, 2H), 3.90-3.58 (m, 4H), 3.55-3.39 (m, 2H), 2.34 (s, 3H), 2.24-2.14 (m, 1H), 1.91-1.77 (m, 2H), 1.72-1.55 (m, 3H), 1.30 (s, 3H), 1.21 (s, 3H), 1.00 (s, 3H). LCMS (ES, m/z): 532 [M+H]+, Rt 1.053 min.

### Example 387: 4-[[(1R,9R)-8-[6-[(dimethylamino)methyl]-2-pyridyl]-1,11,11-trimethyl-12-oxa-4,6,8-triazatricyclo[7.4.0.02,7]trideca-2(7),3,5-trien-5-yl]amino]-1-tetrahydropyran-4-yl-pyridin-2-one

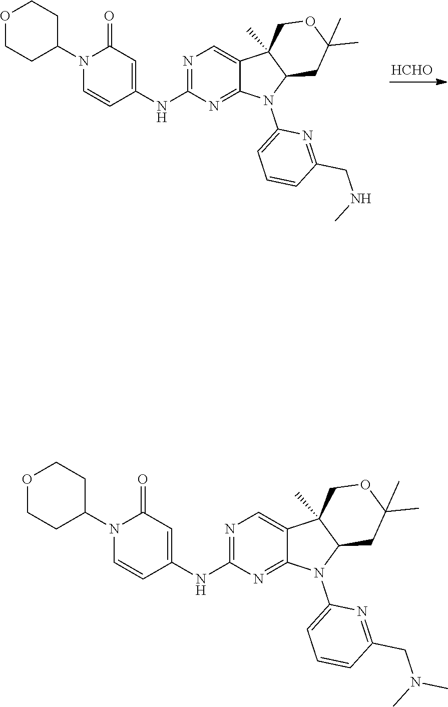

To a stirred solution of 1-tetrahydropyran-4-yl-4-[[(1R,9R)-1,11,11-trimethyl-8-[6-(methylaminomethyl)-2-pyridyl]-12-oxa-4,6,8-triazatricyclo[7.4.0.02,7]trideca-2(7),3,5-trien-5-yl]amino]pyridin-2-one (Example 386, 25 mg, 47.0 μmol, 1 eq.), HCHO (37% in water, 43 mg, 470 μmol, 10 eq.), TFA (80 mg, 705.4 μmol, 15 eq.) in EtOH (1 mL) was added sodium cyanoborohydride (29 mg, 470 μmol, 10 eq.) dropwise at 0° C. The resulting mixture was stirred for 2 h at room temperature under N₂ atmosphere. The resulting mixture was concentrated under reduced pressure. The crude product was purified by Prep-HPLC (Column: XBridge Prep OBD C18 Column, 30*150 mm, 5 μm; Mobile Phase A: Water(10 mmol/L NH₄HCO₃+0.1% NH₃·H₂O), Mobile Phase B: ACN; Flow rate: 60 mL/min; Gradient: 20% B to 50% B in 7 min, 50% B; Wave Length: 254 nm) to afford 4-[[(1R,9R)-8-[6-[(dimethylamino)methyl]-2-pyridyl]-1,11,11-trimethyl-12-oxa-4,6,8-triazatricyclo[7.4.0.02,7]trideca-2(7),3,5-trien-5-yl]amino]-1-tetrahydropyran-4-yl-pyridin-2-one (15.8 mg, 28.9 μmol, 61.5% yield) as a white solid. LCMS (ES, m/z): 546 [M+H]⁺. Rt 0.555 min. ¹H NMR (400 MHz, DMSO-d6) δ 9.60 (s, 1H), 8.46 (d, J=6.4 Hz, 1H), 8.13 (s, 1H), 7.80 (t, J=7.6 Hz, 1H), 7.60 (d, J=7.6 Hz, 1H), 7.11-7.10 (m, 2H), 6.58-6.52 (m, 1H), 4.90-4.83 (m, 1H), 4.70-4.67 (m, 1H), 3.99-3.95 (m, 2H), 3.80-3.77 (m, 1H), 3.68-3.65 (m, 1H), 3.54-3.42 (m, 4H), 2.21-2.16 (m, 7H), 1.89-1.79 (m, 2H), 1.72-1.63 (m, 3H), 1.30 (s, 3H), 1.20 (s, 3H), 0.99 (s, 3H).

TABLE 21
Using appropriate intermediates and reaction conditions, the following examples were prepared in a similar manner
to Example 386.

| Structure | EX # | ¹H NMR | LCMS |
|---|---|---|---|
| 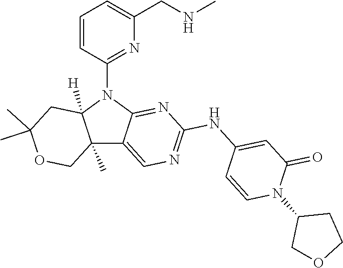 [SMILES: CNCc1cccc(N2c3nc(Nc4ccn([C@@H]5CCOC5)c(=O)c4)ncc3[C@@]3(C)COC(C)(C)C[C@@H]23)n1] | 388, 389 | 1H NMR (400 MHz, DMSO-d6) δ 9.61 (s, 1H), 8.44 (d, J = 8.4 Hz, 1H), 8.13 (s, 1H), 7.70 (t, J = 8.4 Hz, 1H), 7.42 (d, J = 8.0 Hz, 1H), 7.15-7.05 (m, 2H), 6.58-6.51 (m, 1H), 5.48-5.10 (m, 1H), 4.76-4.52 (m, 1H), 4.12-3.98 (m, 1H), 3.87- 3.63 (m, 7H), 2.44-2.33 (m, 4H), 2.24-2.15 (m, 1H), 2.00-1.85 (m, 1H), 1.75-1.52(m, 1H), 1.30 (s, 3H), 1.21 (s, 3H), 1.00 (s, 3H). | LCMS (ES, m/z): 518.20 [M + H]+, Rt 1.070 min. |
| 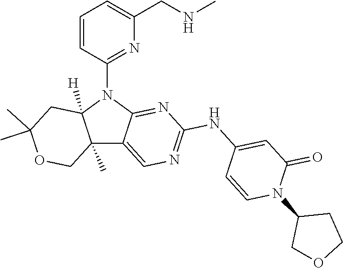 [SMILES: CNCc1cccc(N2c3nc(Nc4ccn([C@H]5CCOC5)c(=O)c4)ncc3[C@@]3(C)COC(C)(C)C[C@@H]23)n1] | | 1H NMR (400 MHz, DMSO-d6) δ 9.61 (s, 1H), 8.44 (d, J = 8.4 Hz, 1H), 8.13 (s, 1H), 7.81-7.71 (m, 1H), 7.43 (d, J = 7.6 Hz, 1H), 7.16- 7.05 (m, 2H), 6.58-6.51 (m, 1H), 5.38-5.27 (m, 1H), 4.75-4.68 (m, 1H), 4.09-3.97 (m, 1H), 3.88-3.59 (m, 7H), 2.44-2.25 (m, 4H), 2.24- 2.13 (m, 1H), 2.00-1.86 (m, 1H), 1.73-1.60 (m, 1H), 1.30 (s, 3H), 1.21 (s, 3H), 1.00 (s, 3H). | LCMS (ES, m/z): 518.20 [M + H]+, Rt 1.080 min. |
| 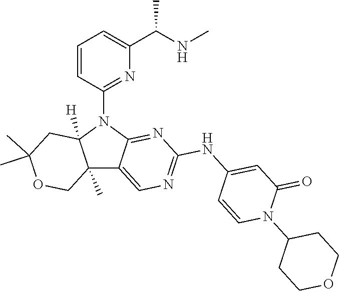 [SMILES: CN[C@@H](C)c1cccc(N2c3nc(Nc4ccn(C5CCOCC5)c(=O)c4)ncc3[C@@]3(C)COC(C)(C)C[C@@H]23)n1] | 390, 391 | 1H NMR (400 MHz, DMSO-d6) δ 9.61 (s, 1H), 8.47 (d, J = 8.4 Hz, 1H), 8.14 (s, 1H), 7.82 (t, J = 8.0 Hz, 1H), 7.59 (d, J = 7.6 Hz, 1H), 7.16-7.08 (m, 2H), 7.13-7.10 (m, 1H), 4.92-4.81 (m, 1H), 4.79-4.72 (m, 1H), 4.39-4.01 (m, 2H), 3.88- 3.74 (m, 2H), 3.70-3.63 (m, 1H), 3.52-3.39 (m, 2H), 2.48-2.42 (m, 1H), 2.33 (s, 3H), 2.20-2.11 (m, 1H), 1.93-1.76 (m, 2H), 1.72-1.61 (m, 3H), 1.38-1.29 (m, 6H), 1.22 (s, 3H), 1.00 (s, 3H). | LCMS (ES, m/z): 546.30 [M + H]+, Rt 1.068 min. |
| 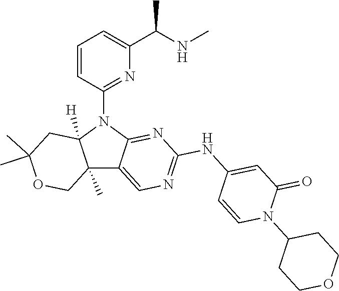 [SMILES: CN[C@H](C)c1cccc(N2c3nc(Nc4ccn(C5CCOCC5)c(=O)c4)ncc3[C@@]3(C)COC(C)(C)C[C@@H]23)n1] | | 1H NMR (400 MHz, DMSO-d6) δ 9.61 (s, 1H), 8.45 (d, J = 8.4 Hz, 1H), 8.13 (s, 1H), 7.78 (t, J = 8.0 Hz, 1H), 7.59 (d, J = 7.6 Hz, 1H), 7.14-7.05 (m, 2H), 6.59-6.52 (m, 1H), 4.94-4.70 (m, 2H), 4.02-3.93 (m, 2H), 3.82-3.75 (m, 1H), 3.71- 3.58 (m, 2H), 3.50-3.40 (m, 2H), 2.25-2.15 (m, 4H), 1.92-1.77 (m, 2H), 1.73-1.60 (m, 3H), 1.34-1.29 (m, 6H), 1.22 (s, 3H), 1.01 (s, 3H). | LCMS (ES, m/z): 546.30 [M + H]+, Rt 1.147 min. |
| 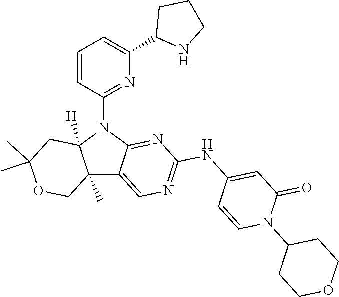 | 392, 394 | 1H NMR (300 MHz, DMSO-d6) δ 9.61 (s, 1H), 8.44 (d, J = 8.1 Hz, 1H), 8.11 (s, 1H), 7.78-7.73 (m, 1H), 7.58 (d, J = 7.5 Hz, 1H), 7.16- 7.10 (m, 2H), 6.57-6.53 (m, 1H), 4.86-4.79 (m, 1H), 4.68-4.66 (m, 1H), 4.13 (t, J = 6.6 Hz, 1H), 3.99- 3.94 (m, 2H), 3.81-3.77 (m, 1H), 3.69-3.65 (m, 1H), 3.48-3.41 (m, 2H), 3.06-2.98 (m, 1H), 2.89-2.82 (m, 1H), 2.30-2.11 (m, 2H), 1.86- 1.62 (m, 8H), 1.30 (s, 3H), 1.28 (s, 3H), 1.02 (s, 3H). | LCMS (ES, m/z): 558.30 [M + H]+, Rt 0.558 min. |
| 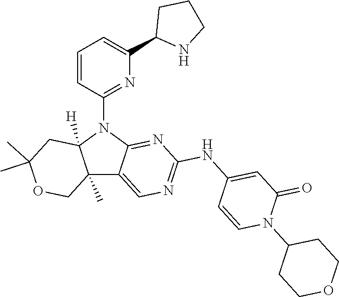 [SMILES: CC1(C)C[C@H]2N(c3cccc([C@H]4CCCN4)n3)c3nc(Nc4ccn(C5CCOCC5)c(=O)c4)ncc3[C@@]2(C)CO1] | | 1H NMR (400 MHz, DMSO-d6) δ 9.60 (s, 1H), 8.44 (d, J = 8.4 Hz, 1H), 8.12 (s, 1H), 7.75 (t, J = 8.0 Hz, 1H), 7.58 (d, J = 7.6 Hz, 1H), 7.21-6.99 (m, 2H), 6.69-6.45(m, 1H), 5.01-4.80 (m, 1H), 4.80-4.60 (m, 1H), 4.20-4.06 (m, 1H), 4.06- 3.87 (m, 2H), 3.87-3.76 (m, 1H), 3.76-3.64 (m, 1H), 3.50-3.40 (m, 2H), 3.15-2.99 (m, 1H), 2.91-2.80 (m, 1H), 2.27-2.18 (m, 1H), 2.15- 2.05 (m, 1H), 1.91-1.83 (m, 1H), 1.95-1.59 (m, 8H), 1.31 (s, 3H), 1.22 (s, 3H), 1.02 (s, 3H). | LCMS (ES, m/z): 558.30 [M + H]+, Rt 0.567 min. |
| 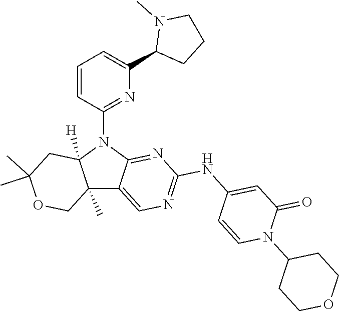 [SMILES: CN1CCC[C@H]1c1cccc(N2c3nc(Nc4ccn(C5CCOCC5)c(=O)c4)ncc3[C@@]3(C)COC(C)(C)C[C@@H]23)n1] | 393, 395 | 1H NMR (300 MHz, DMSO-d6) δ 9.60 (s, 1H), 8.44 (d, J = 8.4 Hz, 1H), 8.12 (s, 1H), 7.79 (t, J = 7.8 Hz, 1H), 7.58 (d, J = 7.8 Hz, 1H), 7.13-7.10 (m, 2H), 6.57-6.53 (m, 1H), 4.96-4.87 (m, 1H), 4.68-4.65 (m, 1H), 3.99-3.95 (m, 2H), 3.80- 3.64 (m, 2H), 3.48-3.41 (m, 2H), 3.32-3.25 (m, 1H), 3.14-3.10 (m, 1H), 2.34-2.16 (m, 6H), 1.86-81 (m, 5H), 1.77-1.63 (m, 3H), 1.30 (s, 3H), 1.21 (s, 3H), 0.99 (s, 3H). | LCMS (ES, m/z): 572.30 [M + H]+, Rt 0.558 min. |
| 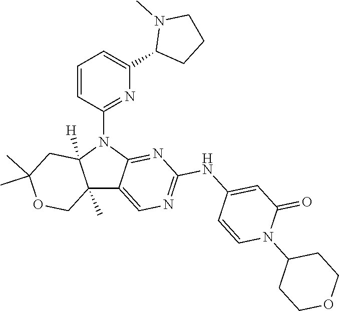 [SMILES: CN1CCC[C@@H]1c1cccc(N2c3nc(Nc4ccn(C5CCOCC5)c(=O)c4)ncc3[C@@]3(C)COC(C)(C)C[C@@H]23)n1] | | 1H NMR (400 MHz, DMSO-d6) δ 9.60 (s, 1H), 8.45 (d, J = 8.4 Hz, 1H), 8.12 (s, 1H), 7.79 (t, J = 8.0 Hz, 1H), 7.58 (d, J = 7.6 Hz, 1H), 7.13-7.07 (m, 2H), 6.59-6.52 (m, 1H), 4.93-4.81 (m, 1H), 4.72-4.65 (m, 1H), 4.01-3.93 (m, 2H), 3.82- 3.74 (m, 1H), 3.71-3.63 (m, 1H), 3.45 (t, J = 10.8 Hz, 2H), 3.24- 3.17 (m, 1H), 3.17-3.08 (m, 1H), 2.35-2.26 (m, 1H), 2.23-2.14 (m, 5H), 1.94-1.75 (m, 5H), 1.74-1.60 (m, 3H), 1.31 (s, 3H), 1.21 (s, 3H), 1.00 (s, 3H). | LCMS (ES, m/z): 572.30 [M + H]+, Rt 1.078 min. |

### Example 519. (4bR,8aR)-4b,7,7-trimethyl-9-(1-methyl-1H-pyrazol-3-yl)-N-(4-((1S,4S)-5-methyl-2,5-diazabicyclo[2.2.1]heptan-2-yl)phenyl)-4b,5,7,8,8a,9-hexahydropyrano[3′,4′:4,5]pyrrolo[2,3-d]pyrimidin-2-amine

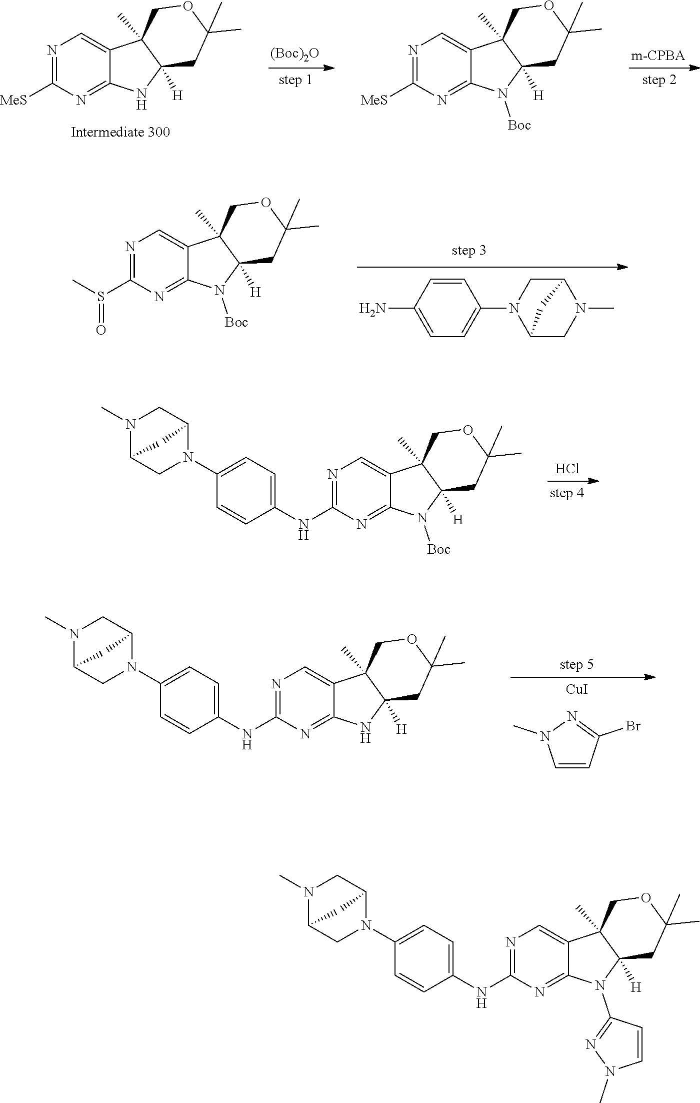

### Step 1. tert-butyl (4bR,8aR)-4b,7,7-trimethyl-2-(methylthio)-4b,7,8,8a-tetrahydropyrano[3′,4′:4,5]pyrrolo[2,3-d]pyrimidine-9(5H)-carboxylate

To a stirred solution of (4bR,8aR)-4b,7,7-trimethyl-2-(methylthio)-4b,5,7,8,8a,9-hexahydropyrano[3′,4′:4,5]pyrrolo[2,3-d]pyrimidine (Intermediate 300, 300 mg, 1.13 mmol, 1 eq.) and TEA (343.18 mg, 3.39 mmol, 3 eq.) and DMAP (69.05 mg, 565.24 μmol, 5 eq.) in DCM (5 mL) was added Boc₂O (296.07 mg, 1.36 mmol, 1.2 eq.) at 0° C. The resulting mixture was stirred for 4 h at 25° C. under N₂ atmosphere and then was concentrated under reduced pressure. The residue was purified by silica gel column chromatography, eluted with EA/PE (1:1) to afford tert-butyl (4bR,8aR)-4b,7,7-trimethyl-2-(methylthio)-4b,7,8,8a-tetrahydropyrano[3′,4′:4,5]pyrrolo[2,3-d]pyrimidine-9(5H)-carboxylate (350 mg, 960.36 μmol, 85% yield, 90% purity) as a yellow solid. LCMS (ES, m/z): 366 [M+H]⁺, Rt 0.739 min.

### Step 2. tert-butyl (4bR,8aR)-4b,7,7-trimethyl-2-(methylsulfinyl)-4b,7,8,8a-tetrahydropyrano[3′,4′:4,5]pyrrolo[2,3-d]pyrimidine-9(5H)-carboxylate

To a stirred solution of tert-butyl (4bR,8aR)-4b,7,7-trimethyl-2-(methylthio)-4b,7,8,8a-tetrahydropyrano[3′,4′:4,5]pyrrolo[2,3-d]pyrimidine-9(5H)-carboxylate (0.2 g, 547.21 μmol, 1 eq.) in DCM (3 mL) was added m-CPBA (103 mg, 601.93 μmol, 1.1 eq.) in portions. The mixture was stirred at room temperature for 2 h and then was concentrated under reduced pressure to afford tert-butyl (4bR,8aR)-4b,7,7-trimethyl-2-(methylsulfinyl)-4b,7,8,8a-tetrahydropyrano[3′,4′:4,5]pyrrolo[2,3-d]pyrimidine-9(5H)-carboxylate (190 mg, crude) as a yellow solid. LCMS (ES, m/z): 382 [M+H]⁺, Rt 0.625 min.

### Step 3. tert-butyl (4bR,8aR)-4b,7,7-trimethyl-2-((4-((1S,4S)-5-methyl-2,5-diazabicyclo[2.2.1]heptan-2-yl)phenyl)amino)-4b,7,8,8a-tetrahydropyrano[3′,4′:4,5]pyrrolo[2,3-d]pyrimidine-9(5H)-carboxylate

To the mixture of tert-butyl (1R,9R)-1,11,11-trimethyl-5-methylsulfinyl-12-oxa-4,6,8-triazatricyclo[7.4.0.02,7]trideca-2,4,6-triene-8-carboxylate (170 mg, 445.62 μmol, 1 eq.) in toluene (2 mL) was added DIEA (0.5 mL) and 4-((1S,4S)-5-methyl-2,5-diazabicyclo[2.2.1]heptan-2-yl)aniline (Intermediate 54, 181.17 mg, 891.25 μmol, 2 eq.). The mixture was stirred for 16 h at 100° C. and then was allowed to cool down to room temperature. The resulting mixture was concentrated under reduced pressure. The residue was purified by (Column: C18; Mobile phase, A: water (containing 10 mmol/L NH₄HCO₃) and B: MeOH (65% B over 5 min); Detector: UV 254 nm) to afford afford tert-butyl (4bR,8aR)-4b,7,7-trimethyl-2-((4-((1S,4S)-5-methyl-2,5-diazabicyclo[2.2.1]heptan-2-yl)phenyl)amino)-4b,7,8,8a-tetrahydropyrano[3′,4′:4,5]pyrrolo[2,3-d]pyrimidine-9(5H)-carboxylate (190 mg, 363.00 μmol, 81% yield) as a yellow oil. LCMS (ES, m/z): 521 [M+H]⁺, Rt 0.533 min.

### Step 4. (4bR,8aR)-4b,7,7-trimethyl-N-(4-((1S,4S)-5-methyl-2,5-diazabicyclo[2.2.1]heptan-2-yl)phenyl)-4b,5,7,8,8a,9-hexahydropyrano[3′,4′:4,5]pyrrolo[2,3-d]pyrimidin-2-amine

A mixture of tert-butyl (4bR,8aR)-4b,7,7-trimethyl-2-((4-((1S,4S)-5-methyl-2,5-diazabicyclo[2.2.1]heptan-2-yl)phenyl)amino)-4b,7,8, 8a-tetrahydropyrano[3′,4′:4,5]pyrrolo[2,3-d]pyrimidine-9(5H)-carboxylate (210 mg, 403.33 μmol, 1 eq.) and HCl (6M, 2 mL) in 1,4-dioxane (5 mL) was stirred for 2 h at room temperature. The resulting mixture was concentrated under reduced pressure to afford (4bR,8aR)-4b,7,7-trimethyl-N-(4-((1S,4S)-5-methyl-2,5-diazabicyclo[2.2.1]heptan-2-yl)phenyl)-4b,5,7,8,8a,9-hexahydropyrano[3′,4′:4,5]pyrrolo[2,3-d]pyrimidin-2-amine (130 mg, crude) as a yellow oil. LCMS (ES, m/z): 421 [M+H]⁺, Rt 0.42 min.

### Step 5. (4bR,8aR)-4b,7,7-trimethyl-9-(1-methyl-1H-pyrazol-3-yl)-N-(4-((1S,4S)-5-methyl-2,5-diazabicyclo[2.2.1]heptan-2-yl)phenyl)-4b,5,7,8,8a,9-hexahydropyrano[3′,4′:4,5]pyrrolo[2,3-d]pyrimidin-2-amine

To a stirred mixture of (4bR,8aR)-4b,7,7-trimethyl-N-(4-((1S,4S)-5-methyl-2,5-diazabicyclo[2.2.1]heptan-2-yl)phenyl)-4b,5,7,8,8a,9-hexahydropyrano[3′,4′:4,5]pyrrolo[2,3-d]pyrimidin-2-amine (120 mg, 285.34 μmol, 1 eq.) and 3-bromo-1-methyl-pyrazole (92 mg, 570.68 μmol, 1 eq.) in 1,4-dioxane (1.5 mL) was added CuI (27 mg, 142.67 μmol, 0.5 eq.), N,N′-dimethyl-1,2-ethanediamine (25 mg, 285.34 μmol, 1 eq.) and K₂CO₃ (118 mg, 856.02 μmol, 3 eq.) in portions. The reaction was stirred for 16 h at 100° C. under the N₂ atmosphere. The mixture was allowed to cool down to room temperature. The solids were filtered out and washed with EA (2×10 mL). The filtrate was concentrated under reduced pressure. The residue was purified by reverse phase chromatography (Column: Xselect CSH C18 OBD 30*150 mm/5 μm, Mobile Phase A: Water (10 mmol/L NH₄HCO₃+0.1% NH₃—H₂O), Mobile Phase B: ACN; Flow rate: 60 mL/min; Gradient: 30% to 50% B in 7 min; Wave Length: 254 nm; RT: 6 min) to afford (4bR,8aR)-4b,7,7-trimethyl-9-(1-methyl-1H-pyrazol-3-yl)-N-(4-((1S,4S)-5-methyl-2,5-diazabicyclo[2.2.1]heptan-2-yl)phenyl)-4b,5,7,8,8a,9-hexahydropyrano[3′,4′:4,5]pyrrolo[2,3-d]pyrimidin-2-amine (28.9 mg, 56.80 μmol, 20% yield) as a white solid.

¹H NMR (300 MHz, DMSO-d6) δ 8.72 (s, 1H), 7.82 (s, 1H), 7.62 (d, J=2.4 Hz, 1H), 7.43 (d, J=8.8 Hz, 2H), 6.88 (s, 1H), 6.50 (d, J=8.8 Hz, 2H), 4.28-4.18 (m, 2H), 3.77 (s, 3H), 3.73-3.55 (m, 2H), 3.42-3.36 (m, 1H), 3.32-3.26 (m, 1H), 3.14-3.05 (m, 1H), 2.81-2.72 (m, 1H), 2.60-2.52 (m, 1H), 2.23 (s, 3H), 2.15-2.05 (m, 1H), 1.88-1.71 (m, 3H), 1.23 (s, 3H), 1.19 (s, 3H), 1.00 (s, 3H). LCMS (ES, m/z): 501 [M+H]⁺, Rt 0.585 min.

Using appropriate intermediates and reaction conditions, the following examples in Table 22 were prepared in the similar manner to Example 519.

TABLE 22

| Structure | EX # | ¹H NMR | LCMS |
|---|---|---|---|
| 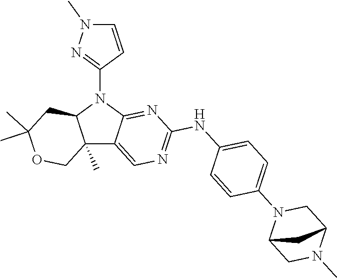 | 520 | 1H NMR (300 MHz, DMSO-d6) δ 8.74 (s, 1H), 7.83 (s, 1H), 7.62 (d, J = 2.4 Hz, 1H), 7.44 (d, J = 8.7 Hz, 2H), 6.88 (s, 1H), 6.51 (d, J = 9.0 Hz, 2H), 4.28-4.18 (m, 2H), 3.78 (s, 3H), 3.73-3.55 (m, 2H), 3.42-3.36 (m, 1H), 3.32-3.26 (m, 1H), 3.14-3.08 (m, 1H), 2.80-2.70 (m, 1H), 2.60-2.52 (m, 1H), 2.25 (s, 3H), 2.15-2.05 (m, 1H), 1.88- 1.71 (m, 3H), 1.23 (s, 3H), 1.17 (s, 3H), 1.00 (s, 3H). | LCMS (ES, m/z): 501.25 [M + H]+, Rt 0.583 min. |
| 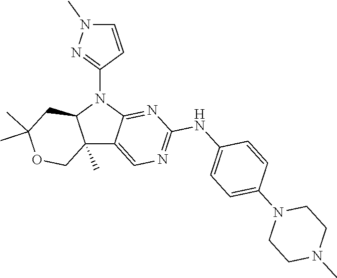 | 717 | 1H NMR (300 MHz, DMSO-d6) δ 8.87 (s, 1H), 7.85 (s, 1H), 7.77- 7.39 (m, 3H), 7.00-6.81 (m, 3H), 4.40-4.15 (m, 1H), 3.78 (s, 3H), 3.79-3.55 (m, 2H), 3.19-2.90 (m, 4H), 2.50-2.38 (m, 4H), 2.22 (s, 3H), 2.16-2.00(m, 1H), 1.99-1.76 (m, 1H), 1.24 (s, 3H), 1.17 (s, 3H), 1.00 (s, 3H). | LCMS (ES, m/z): 489.25 [M + H]+, Rt 0.575 min. |
| 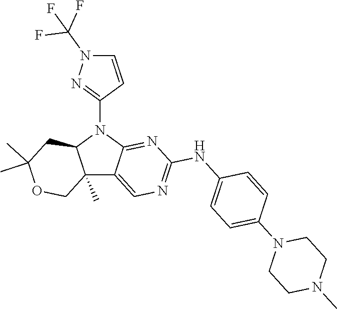 | 719 | 1H NMR (400 MHz, DMSO-d6) δ 9.09 (s, 1H), 8.48 (d, J = 3.2 Hz, 1H), 8.00 (s, 1H), 7.53-7.43 (m, 3H), 6.92-6.85 (m, 2H), 4.30-4.27 (m, 1H), 3.74-3.72 (m, 1H), 3.62- 3.59 (m, 1H), 3.06 (t, J = 4.8 Hz, 4H), 2.46 (t, J = 4.8 Hz, 4H), 2.23 (s, 3H), 2.12-2.07 (m, 1H), 1.96- 1.90 (m, 1H), 1.26 (s, 3H), 1.18 (s, 3H), 0.97 (s, 3H). | LCMS (ES, m/z): 543.25 [M + H]+, Rt 0.695 min. |
| 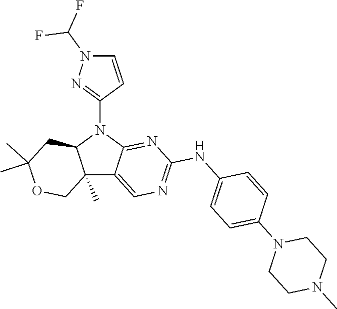 | 720 | 1H NMR (400 MHz, DMSO-d6) δ 9.02 (s, 1H), 8.21 (d, J = 2.8 Hz, 1H), 7.96 (s, 1H), 7.71-7.52 (m, 3H), 7.34-7.29 (m, 1H), 6.92-6.87 (m, 2H), 4.30-4.27 (m, 1H), 3.71- 3.68 (m, 1H), 3.62-3.59 (m, 1H), 3.06 (t, J = 4.8 Hz, 4H), 2.45 (t, J = 4.8 Hz, 4H), 2.22 (s, 3H), 2.13- 2.08 (m, 1H), 1.94-1.88 (m, 1H), 1.26 (s, 3H), 1.18 (s, 3H), 0.98 (s, 3H). | LCMS (ES, m/z): 525.25 [M + H]+, Rt 0.628 min. |
| 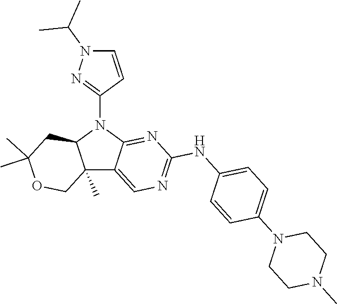 | 721 | 1H NMR (400 MHz, DMSO-d6) δ 8.87 (s, 1H), 7.84 (s, 1H), 7.71 (s, 1H), 7.55 (d, J = 8.4 Hz, 2H), 7.00-6.75 (m, 3H), 4.56-4.35 (m, 1H), 4.35-4.15 (m, 1H), 3.70-3.55 (m, 2H), 3.11-3.02 (m, 4H), 2.50- 2.44 (m, 4H), 2.25 (s, 3H), 2.13- 2.04 (m, 1H), 1.95-1.85 (m, 1H), 1.41 (d, J =6.4 Hz, 6H), 1.26 (s, 3H), 1.17 (s, 3H), 0.98 (s, 3H). | LCMS (ES, m/z): 517.30 [M + H]+, Rt 1.218 min. |
| 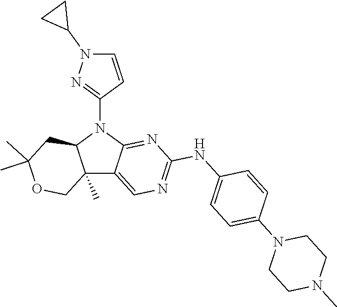 | 739 | 1H NMR (400 MHz, DMSO-d6) δ 8.88 (s, 1H), 7.85 (s, 1H), 7.72 (s, 1H), 7.54 (d, J = 8.8 Hz, 2H), 6.99- 6.82 (m, 3H), 4.37-4.15 (m, 1H), 3.71-3.54 (m, 3H), 3.08-3.01 (m, 4H), 2.49-2.42 (m, 4H), 2.20 (s, 3H), 2.13-2.04 (m, 1H), 1.89-1.79 (m, 1H), 1.24 (s, 3H), 1.18 (s, 3H), 1.05-0.89 (m, 7H). | LCMS (ES, m/z): 515.20 [M + H]+, Rt 1.416 min. |
| 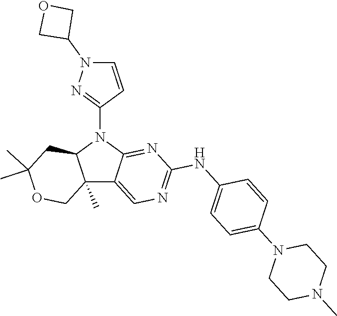 | 742 | 1H NMR (300 MHz, DMSO-d6) δ 8.92 (s, 1H), 7.88-7.85 (m, 2H), 7.56 (d, J = 9.0 Hz, 2H), 6.99 (d, J = 1.8 Hz, 1H), 7.47 (d, J = 9.0 Hz, 2H), 5.53-5.48 (m, 1H), 4.91- 4.88 (m, 4H), 4.33-4.29 (m, 1H), 3.65-3.61 (m, 2H), 3.06-3.03 (m, 4H), 2.49-2.45 (m, 4H), 2.21 (s, 3H), 2.15-2.10 (m, 1H), 1.98-1.92 (m, 1H), 1.26 (s, 3H), 1.18 (s, 3H), 0.98 (s, 3H). | LCMS (ES, m/z): 531.30 [M + H]+, Rt 0.562 min. |
| 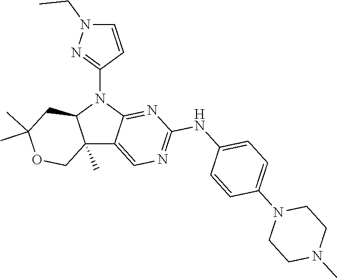 | 744 | 1H NMR (400 MHz, DMSO-d6) δ 8.90 (s, 1H), 7.86 (s, 1H), 7.70 (d, J = 2.4 Hz, 1H), 7.56 (d, J = 8.8 Hz, 2H), 6.91-6.82 (m, 3H), 4.29- 4.21 (m, 1H), 4.12-4.02 (m, 2H), 3.71-3.56 (m, 2H), 3.09-3.02 (m, 4H), 2.49-2.42 (m, 4H), 2.23 (s, 3H), 2.14-2.04 (m, 1H), 1.94-1.82 (m, 1H), 1.37 (t, J = 7.2 Hz, 3H), 1.24 (s, 3H), 1.17 (s, 3H), 0.99 (s, 3H). | LCMS (ES, m/z): 503.30 [M + H]+, Rt 0.633 min. |

### Examples 787 and 788. (R)-7-ethyl-2-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)-6,7-dihydro-5H-cyclopenta[b]pyridin-7-ol and (S)-7-ethyl-2-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)-6,7-dihydro-5H-cyclopenta[b]pyridin-7-ol

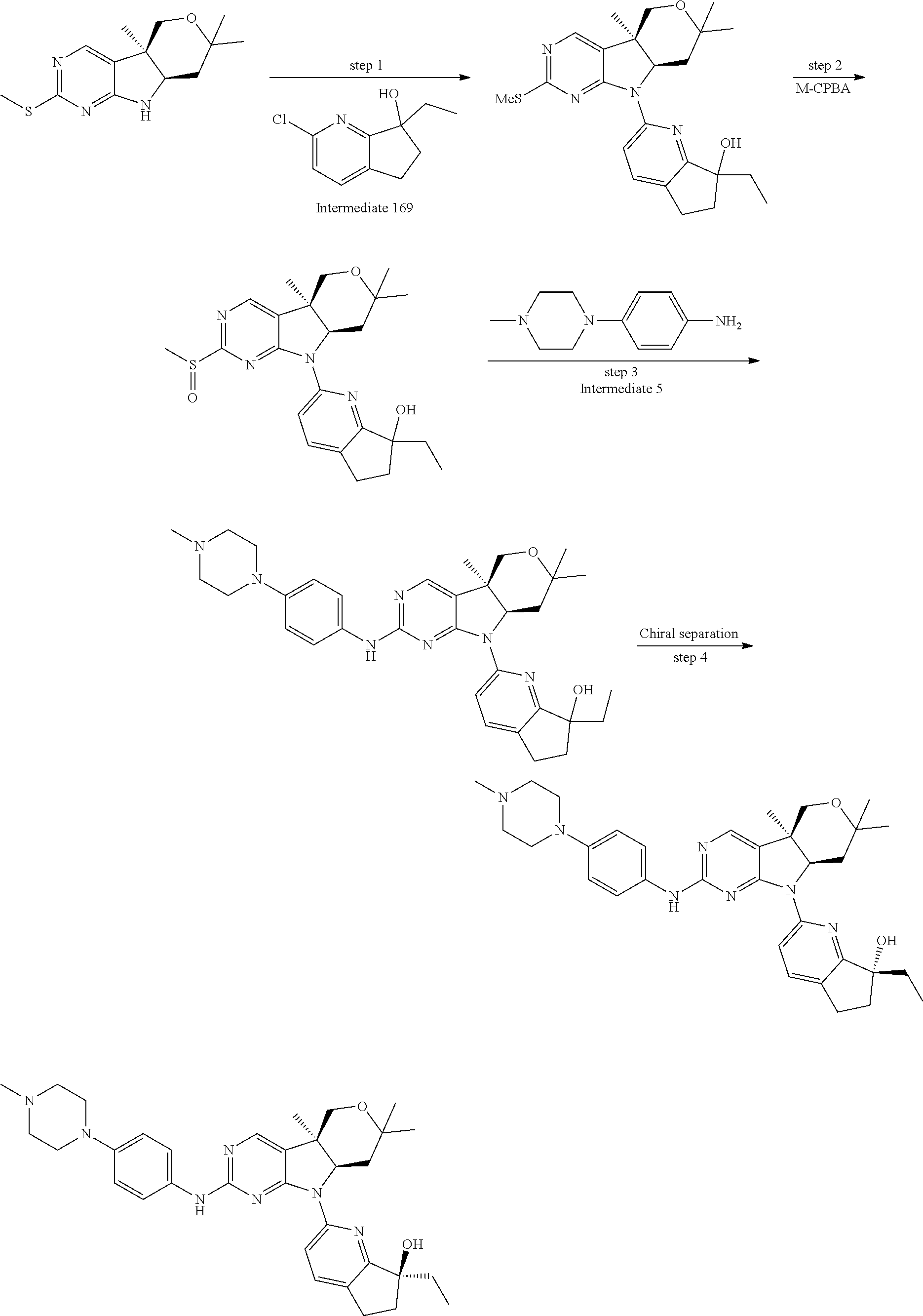

### Step 1. 7-ethyl-2-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)-6,7-dihydro-5H-cyclopenta[b]pyridin-7-ol

### To a Stirred Solution of (4bR,8aR)-4b,7,7-trimethyl-2-(methylthio)-4b,5,7,8,8a,9-hexahydropyrano[3′,4′:4,5]pyrrolo[2,3-d]pyrimidine (Intermediate 300, 150 mg, 0.56 mmol, 1 eq.) and 2-chloro-7-ethyl-6,7-dihydro-5H-cyclopenta[b]pyridin-7-ol

(Intermediate 169, 134 mg, 0.68 mmol, 1.2 eq.) in 1,4-dioxane were added Pd₂(dba)₃ (51 mg, 56.52 μmol, 0.1 eq.), BINAP (71 mg, 0.11 mmol, 0.2 eq.) and Cs₂CO₃ (552 mg, 1.70 mmol, 3 eq.). The resulting mixture was stirred for 2 h at 100° C. under the N₂ atmosphere and then was allowed to cool down to room temperature. The solids were filtered and washed with EA (3×10 mL). The filtrate was concentrated under reduced pressure. The residue was purified by silica gel column chromatography (eluting with 1:5 PE/EA) to afford 7-ethyl-2-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)-6,7-dihydro-5H-cyclopenta[b]pyridin-7-ol (220 mg, 0.52 mmol, 91% yield) as a yellow solid. LCMS (ES, m/z): 427 [M+H]⁺, Rt 0.671 min.

### Step 2-3. 7-ethyl-2-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)-6,7-dihydro-5H-cyclopenta[b]pyridin-7-ol

Following the general procedures described for Examples 3 and 4/steps 4-5, 7-ethyl-2-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)-6,7-dihydro-5H-cyclopenta[b]pyridin-7-ol (90 mg, 0.158 mmol, 58% yield) was obtained, starting from 7-ethyl-2-((4bR,8aR)-4b,7,7-trimethyl-2-(methylthio)-4b,7,8,8a-tetrahydropyrano[3′,4′:4,5]pyrrolo[2,3-d]pyrimidin-9(5H)-yl)-6,7-dihydro-5H-cyclopenta[b]pyridin-7-ol. LCMS (ES, m/z): 570 [M+H]⁺, Rt 0.542 min.

### Step 4. (R)-7-ethyl-2-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)-6,7-dihydro-5H-cyclopenta[b]pyridin-7-ol and (S)-7-ethyl-2-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)-6,7-dihydro-5H-cyclopenta[b]pyridin-7-ol

The racemic 7-ethyl-2-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)-6,7-dihydro-5H-cyclopenta[b]pyridin-7-ol (90 mg, 0.16 mmol) was separated by Chiral-HPLC (Column: CHIRALPAK ID, 2*25 cm/5 μm; Mobile Phase A: Hex (0.5% 2M NH₃-MeOH), Mobile Phase B: EtOH; Flow rate: 20 mL/min; Gradient: 40% B in 19 min; 220/254 nm; RT1: 11.741 min; RT2: 15.998 min) to afford (R)-7-ethyl-2-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)-6,7-dihydro-5H-cyclopenta[b]pyridin-7-ol (28.6 mg, 32% yield) as a light-yellow solid and (S)-7-ethyl-2-((4bR,8aR)-4b,7,7-trimethyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)-6,7-dihydro-5H-cyclopenta[b]pyridin-7-ol (26.4 mg, 29% yield) as an off-white solid.

Examples 787 and 788: IH NMR (400 MHz, DMSO-d6) δ 9.06 (s, 1H), 8.44 (d, J=8.4 Hz, 1H), 7.95 (s, 1H), 7.64 (d, J=8.4 Hz, 1H), 7.55 (d, J=8.8 Hz, 2H), 6.88 (d, J=9.2 Hz, 2H), 4.75 (s, 1H), 4.61-4.57 (m, 1H), 3.75-3.72 (m, 1H), 3.65-3.62 (m, 1H), 3.07-3.04 (m, 4H), 2.90-2.88 (m, 1H), 2.76-2.62 (m, 1H), 2.49-2.44 (m, 4H), 2.39-2.35 (m, 1H), 2.22 (s, 3H), 2.18-2.09 (m, 1H), 2.03-2.00 (m, 1H), 1.92-1.86 (m, 1H), 1.71-1.65 (m, 2H), 1.26 (s, 3H), 1.21 (s, 3H), 1.01 (s, 3H), 0.93 (t, J=7.6 Hz, 3H). LCMS (ES, m/z): 570 [M+H]⁺, Rt 0.633 min

¹H NMR (400 MHz, DMSO-d6) δ 9.08 (s, 1H), 8.48 (d, J=8.0 Hz, 1H), 7.95 (s, 1H), 7.65 (d, J=8.4 Hz, 1H), 7.55 (d, J=8.8 Hz, 2H), 6.87 (d, J=9.2 Hz, 2H), 4.86 (s, 1H), 4.77-4.73 (m, 1H), 3.75-3.72 (m, 1H), 3.66-3.63 (m, 1H), 3.07-3.05 (m, 4H), 2.91-2.89 (m, 1H), 2.76-2.62 (m, 1H), 2.49-2.44 (m, 4H), 2.22 (s, 3H), 2.15-2.09 (m, 2H), 2.00-1.98 (m, 1H), 1.88-1.85 (m, 1H), 1.79-1.75 (m, 1H), 1.64-1.58 (m, 1H), 1.28 (s, 3H), 1.20 (s, 3H), 0.99 (s, 3H), 0.86 (t, J=7.6 Hz, 3H). LCMS (ES, m/z): 570 [M+H]⁺, Rt 0.648 min.

TABLE 23
Using appropriate intermediates and reaction conditions, the following
examples were prepared in the similar manner to Examples 787 and 788.

| Structure | EX # | ¹H NMR | LCMS |
|---|---|---|---|
| 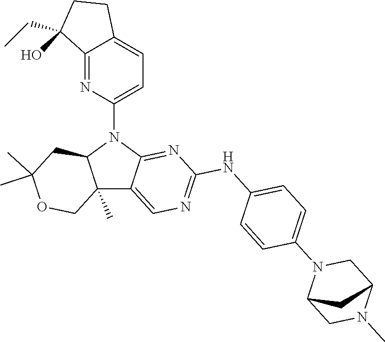 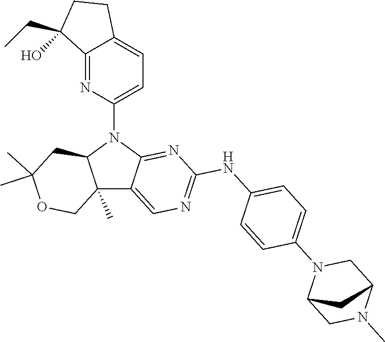 | 789, 790 | 1H NMR (400 MHz, DMSO-d6)δ8.90 (s, 1H), 8.44 (d, J = 8.4 Hz, 1H), 7.92 (s, 1H), 7.60 (d, J = 8.4 Hz, 1H), 7.44 (d, J = 8.8 Hz, 2H), 6.53 (d, J = 8.8 Hz, 2H), 4.75 (s, 1H), 4.59-4.56 (m, 1H), 4.23 (s, 1H), 3.75-3.61 (m, 2H), 3.38-3.34 (m, 1H), 3.32-3.29 (m, 1H), 3.10 (d, J = 8.8 Hz, 1H), 2.89- 2.80 (m, 1H), 2.76-2.74 (m, 1H), 2.70-2.62 (m, 1H), 2.38-2.34 (m, 1H), 2.24 (s, 3H), 2.10-2.02 (m, 1H), 2.00-1.89 (m, 1H), 1.88-1.83 (m, 2H), 1.76-1.65 (m, 3H), 1.25 (s, 3H), 1.24-1.23 (m, 1H), 1.21 (s, 3H), 1.01 (s, 3H), 0.93 (t, J = 7.2 Hz, 3H). 1H NMR (400 MHz, DMSO-d6)δ8.91 (s, 1H), 8.44 (d, J = 8.4 Hz, 1H), 7.92 (s, 1H), 7.61 (d, J = 8.4 Hz, 1H), 7.44 (d, J = 8.8 Hz, 2H), 6.53 (d, J = 8.8 Hz, 2H), 4.86 (s, 1H), 4.75-4.72 (m, 1H), 4.23 (s, 1H), 3.75-3.63 (m, 2H), 3.39-3.34 (m, 1H), 3.32-3.30 (m, 1H), 3.10 (d, J = 9.2 Hz, 1H), 2.89- 2.80 (m, 1H), 2.76-2.74 (m, 1H), 2.70-2.62 (m, 1H), 2.24 (s, 3H), 2.15-2.10 (m, 2H), 2.00-1.97 (m, 1H), 1.88-1.83 (m, 2H), 1.78-1.73 (m, 2H), 1.63-1.60 (m, 1H), 1.28 (s, 3H), 1.24-1.23 (m, 1H), 1.20 (s, 3H), 0.99 (s, 3H), 0.85 (t, J = 3.6 Hz, 3H). | LCMS (ES, m/z): 582.35 [M + H]+, Rt 0.650 min. LCMS (ES, m/z): 582.30 [M + H]+, Rt 0.667 min. |
| 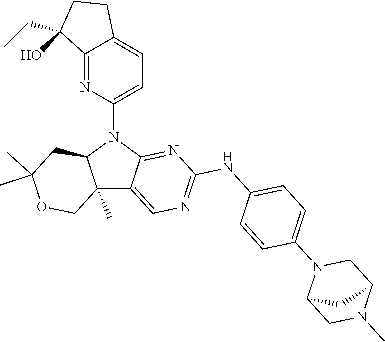 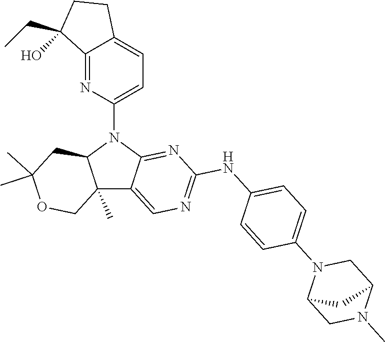 | 791, 792 | 1H NMR (400 MHz, DMSO-d6) δ 8.94 (s, 1H), 8.44 (d, J = 8.4 Hz, 1H), 7.93 (s, 1H), 7.63 (d, J = 8.4 Hz, 1H), 7.49 (d, J = 8.4 Hz, 2H), 6.58 (d, J = 8.4 Hz, 2H), 4.73 (s, 1H), 4.62-4.58 (m, 1H), 4.49-4.36 (m, 1H), 3.82-3.62 (m, 3H), 3.48-3.33 (m, 2H), 3.26-3.22 (m, 1H), 3.03-2.81 (m, 2H), 2.76-2.63 (m, 2H), 2.43-2.89 (m, 2H), 2.15-1.59 (m, 6H), 1.76-1.73 (m, 2H), 1.26 (s, 3H), 1.22 (s, 3H), 1.01 (s, 3H), 0.94 (t, J = 7.4 Hz, 3H). 1H NMR (400 MHz, DMSO-d6) δ 8.96 (s, 1H), 8.49 (d, J = 8.4 Hz, 1H), 7.93 (s, 1H), 7.63 (d, J = 8.4 Hz, 1H), 7.49 (d, J = 8.4 Hz, 2H), 6.58 (d, J = 8.4 Hz, 2H), 4.85 (s, 1H), 4.64-4.73 (m, 1H), 4.22-4.38 (m, 1H), 3.86-3.79 (m, 1H), 3.75-3.72 (m, 1H), 3.66-3.63 (m, 1H), 3.51-3.42 (m, 1H), 3.25-3.22 (m, 1H), 2.95-2.87 (m, 3H), 2.71-2.65 (m, 2H), 2.19-1.18 (m, 8H), 1.82-1.71 (m, 1H), 1.67-1.57 (m, 1H), 1.28 (s, 3H), 1.21 (s, 3H), 0.99 (s, 3H), 0.87 (t, J = 7.4 Hz, 3H). | LCMS (ES, m/z): 582.30 [M + H]+, Rt 0.642 min. LCMS (ES, m/z): 582.30 [M + H]+, Rt 0.658 min. |
| 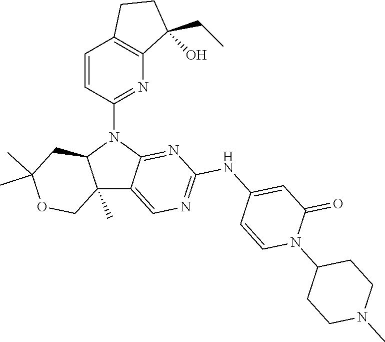 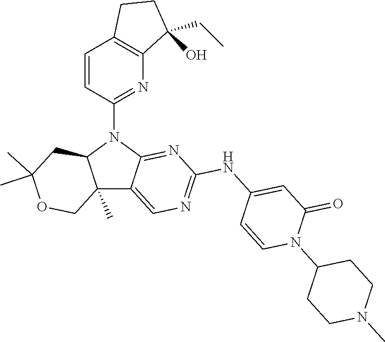 | 811, 812 | 1H NMR (300 MHz, DMSO-d6) δ 9.56 (s, 1H), 8.34 (d, J = 8.4 Hz, 1H), 8.09 (s, 1H), 7.67 (d, J = 8.4 Hz, 1H), 7.54 (d, J = 7.8 Hz, 1H), 7.10 (d, J = 2.4 Hz, 1H), 6.58-6.47 (m, 1H), 4.80 (s, 1H), 4.69-4.51 (m, 2H), 3.85-3.61 (m, 2H), 2.96-2.85 (m, 3H), 2.79-2.62 (m, 1H), 2.41-2.27 (m, 1H), 2.20 (s, 3H), 2.11-1.62 (m, 11H), 1.29 (s, 6H), 1.20 (s, 3H), 1.00 (s, 3H), 0.93 (t, J = 7.5 Hz, 3H). 1H NMR (300 MHz, DMSO-d6) δ 9.57 (s, 1H), 8.41 (d, J = 8.4 Hz, 1H), 8.09 (s, 1H), 7.68 (d, J = 8.4 Hz, 1H), 7.54 (d, J = 7.8 Hz, 1H), 7.09 (d, J = 2.4 Hz, 1H), 6.59-6.50 (m, 1H), 4.88 (s, 1H), 4.84-4.75 (m, 1H), 4.66-4.52 (m, 1H), 3.85-3.61 (m, 2H), 3.00-2.83 (m, 3H), 2.78-2.63 (m, 1H), 2.21 (s, 3H), 2.18-1.95 (m, 5H), 1.94-1.74 (m, 4H), 1.72-1.59 (m, 3H), 1.31 (s, 3H), 1.20 (s, 3H), 0.98 (s, 3H), 0.87 (t, J = 7.2 Hz, 3H). | LCMS (ES, m/z): 586.20 [M + H]+, Rt 0.617 min. LCMS (ES, m/z): 586.25 [M + H]+, Rt 0.633 min. |

TABLE 24
The examples in the following table were synthesized starting from
Intermediate 300 and other appropriate intermediates according to the methods as demonstrated
for Examples 100, 219, 229, 233, 361, 362, 386, 519, 787-788.

| Structure | Ex # | ¹H NMR | LCMS |
|---|---|---|---|
| 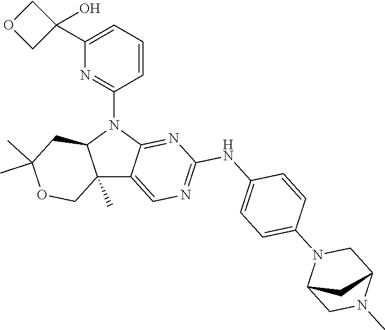 | 517 | 1H NMR (300 MHz, DMSO-d6) δ 8.95 (s, 1H), 8.58 (d, J = 8.4 Hz, 1H), 7.97 (s, 1H), 7.81-7.76 (m, 1H), 7.45 (d, J = 8.7 Hz, 2H), 7.22 (d, J = 7.5 Hz, 1H), 6.67- 6.53 (m, 3H), 5.01 (d, J = 6.0 Hz, 1H), 4.89 (d, J = 6.0 Hz, 1H), 4.74-4.65 (m, 3H), 4.29 (s, 1H), 3.85-3.51 (m, 3H), 3.41-3.33 (m, 1H), 3.15 (d, J = 9.3 Hz, 1H), 2.79-2.75 (m, 1H), 2.69-2.57 (m, 1H), 2.41-2.31 (m, 4H), 1.98-1.78 (m, 2H), 1.72-1.61 (m, 1H), 1.31 (s, 3H), 1.24 (s, 3H), 1.05 (s, 3H). LCMS (ES, m/z): 570 [M + H]+, Rt 0.567 min. | LCMS (ES, m/z): 570 [M + H]+, Rt 0.602 min. |
| 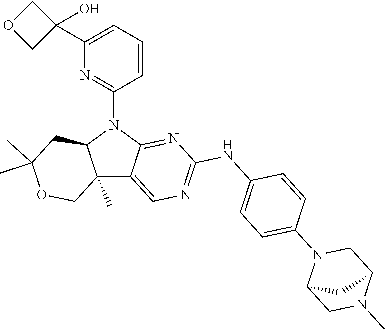 | 518 | 1H NMR (300 MHz, DMSO-d6) δ 8.96 (s, 1H), 8.60 (d, J = 8.4 Hz, 1H), 7.97 (s, 1H), 7.81-7.76 (m, 1H), 7.45 (d, J = 8.7 Hz, 2H), 7.22 (d, J = 7.5 Hz, 1H), 6.67- 6.53 (m, 3H), 5.03 (d, J = 6.0 Hz, 1H), 4.91 (d, J = 6.0 Hz, 1H), 4.74-4.65 (m, 3H), 4.25 (s, 1H), 3.81-3.66 (m, 2H), 3.31 (d, J = 2.1 Hz, 2H), 3.13 (d, J = 9.0 Hz, 1H), 2.79-2.75 (m, 1H), 2.49 (s, 1H), 2.41-2.34 (m, 1H), 2.25 (s, 3H), 1.89- 1.71 (m, 2H), 1.69-1.61 (m, 1H), 1.32 (s, 3H), 1.26 (s, 3H), 1.06 (s, 3H). | LCMS (ES, m/z): 570 [M + H]+, Rt 0.567 min. |
| 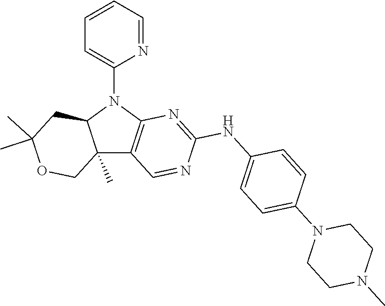 | 722 | 1H NMR (300 MHz, DMSO-d6) δ 9.08 (s, 1H), 8.64 (d, J = 8.4 Hz, 1H), 8.37 (d, J = 5.7 Hz, 1H), 8.01 (s, 1H), 7.82-7.56 (m, 1H), 7.56 (d, J = 8.7 Hz, 2H), 7.05-7.01 (m, 1H), 6.91 (d, J = 9.0 Hz, 2H), 4.70- 4.66 (m, 1H), 3.85-3.73 (m, 1H), 3.71- 3.59 (m, 1H), 3.12-2.98 (m, 4H), 2.48- 2.45 (m, 4H), 2.23-2.13 (m, 4H), 1.65- 1.57 (m, 1H), 1.24 (s, 3H), 1.21 (s, 3H), 0.99 (s, 3H). | LCMS (ES, m/z): 486.25 [M + H]+, Rt 0.608 min. |
| 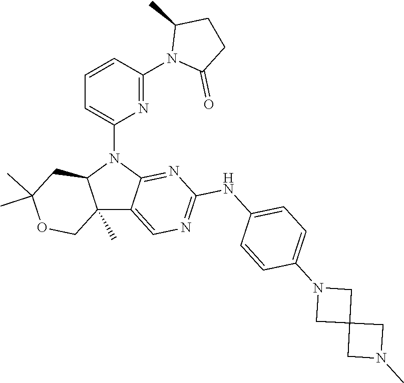 | 725 | 1H NMR (300 MHz, DMSO-d6) δ 8.99 (s, 1H), 8.43-8.34 (m, 1H), 7.97 (s, 1H), 7.85-7.70 (m, 2H), 7.46 (d, J = 8.7 Hz, 2H), 6.39 (d, J = 9.0 Hz, 2H), 4.95-4.69 (m, 1H), 4.68-4.57 (m, 1H), 3.87-3.77 (m, 5H), 3.71-3.61 (m, 1H), 3.29-3.22 (m, 4H), 2.95-2.73 (m, 1H), 2.48-2.36 (m, 1H), 2.36-2.05 (m, 5H), 1.90-1.66 (m, 1H), 1.61-1.47 (m, 1H), 1.45-1.21 (m, 9H), 1.05 (s, 3H). | LCMS (ES, m/z): 595.30 [M + H]+, Rt 0.670 min. |
| 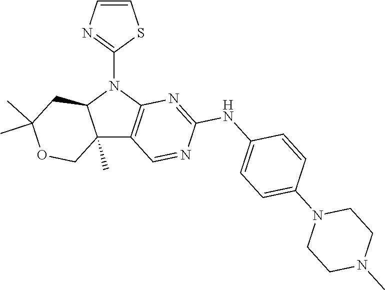 | 726 | 1H NMR (400 MHz, DMSO-d6) δ 9.17 (s, 1H), 8.07 (s, 1H), 7.61 (d, J = 10 Hz, 2H), 7.49 (d, J = 3.6 Hz, 1H), 7.29 (d, J = 3.6 Hz, 1H), 6.91 (d, J = 8.8 Hz, 2H), 4.54- 4.51 (m, 1H), 3.74-3.63 (m, 2H), 3.07 (t, J = 4.4 Hz, 4H), 2.46 (t, J = 4.8 Hz, 4H), 2.24-2.19 (m, 4H), 2.03-1.97 (m, 1H), 1.29 (s, 3H), 1.19 (s, 3H), 0.97 (s, 3H). | LCMS (ES, m/z): 492.25 [M + H]+, Rt 0.587 min. |
| 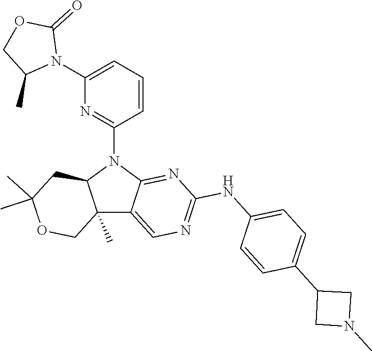 | 727 | 1H NMR (400 MHz, DMSO-d6) δ 9.33 (s, 1H), 8.41 (d, J = 8.0 Hz, 1H), 8.05 (s, 1H), 7.92-7.75 (m, 2H), 7.67 (d, J = 8.0 Hz, 2H), 7.24 (d, J = 8.0 Hz, 2H), 4.89- 4.78 (m, 1H), 4.69-4.60 (m, 1H), 3.88- 3.80 (m, 1H), 3.72-3.65 (m, 1H), 3.64- 3.56 (m, 2H), 3.56-3.45 (m, 1H), 3.08- 2.98 (m, 2H), 2.86-2.72 (m, 1H), 2.48- 2.35 (m, 1H), 2.35-2.24 (m, 4H), 2.24- 2.10 (m, 1H), 1.81-1.66 (m, 1H), 1.63- 1.49 (m, 1H), 1.43-1.15 (m, 9H), 1.06 (s, 3H). | LCMS (ES, m/z): 554.30 [M + H]+, Rt 1.253 min. |
| 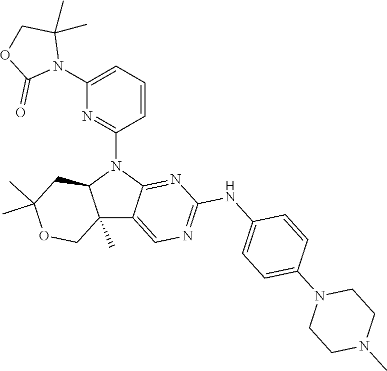 | 728 | 1H NMR (400 MHz, DMSO-d6) δ 9.14 (s, 1H), 8.43 (d, J = 8.4 Hz, 1H), 8.05 (s, 1H), 7.84 (t, J = 8.4 Hz, 1H), 7.55 (d, J = 9.2 Hz, 2H), 7.30 (d, J = 7.6 Hz, 1H), 6.90 (d, J = 9.2 Hz, 2H), 4.63-4.55 (m, 1H), 4.23-4.12 (m, 2H), 3.90-3.83 (m, 1H), 3.72-3.64 (m, 1H), 3.12-3.03 (m, 4H), 2.54-2.43 (m, 4H), 2.23 (s, 3H), 2.17- 2.08 (m, 1H), 1.63 (s, 3H), 1.59 (s, 3H), 1.57-1.49 (m, 1H), 1.24 (s, 3H), 1.22 (s, 3H), 1.03 (s, 3H). | LCMS (ES, m/z): 599 [M + H] +; RT: 1.639 min. |
| 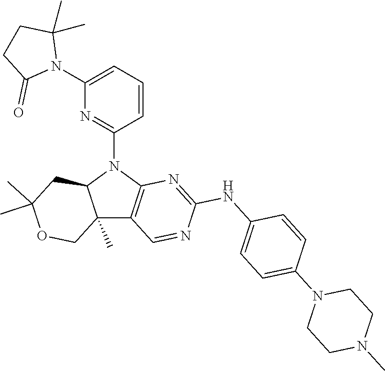 | 729 | 1H NMR (300 MHz, DMSO-d6) δ 9.11 (s, 1H), 8.45 (d, J = 8.1 Hz, 1H), 8.03 (s, 1H), 7.81 (t, J = 8.1 Hz, 1H), 7.55 (d, J = 9.0 Hz, 2H), 7.18 (d, J = 7.8 Hz, 1H), 6.90 (d, J = 9.0 Hz, 2H), 4.63-4.55 (m, 1H), 3.90-3.80 (m, 1H), 3.72-3.62 (m, 1H), 3.13-3.04 (m, 4H), 2.58-2.50 (m, 6H), 2.26 (s, 3H), 2.19-2.07 (m, 1H), 2.01- 1.88 (m, 2H), 1.78-1.40(m, 7H) 1.24 (s, 3H), 1.19 (s, 3H), 1.03 (s, 3H). | LCMS (ES, m/z): 597.30 [M + H]+, Rt 0.662 min. |
| 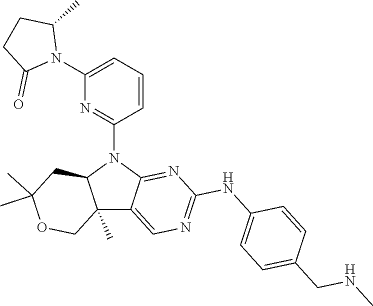 | 730 | 1H NMR (400 MHz, DMSO-d6) δ 9.29 (s, 1H), 8.42 (d, J = 8.0 Hz, 1H), 8.05 (s, 1H), 7.85-7.77 (m, 2H), 7.67 (d, J = 8.4 Hz, 2H), 7.23 (d, J = 8.8 Hz, 2H), 4.86- 4.82 (m, 1H), 4.67-4.64 (m, 1H), 3.85- 3.66 (m, 2H), 3.58 (s, 2H), 2.84-2.75 (m, 1H), 2.47-2.40 (m, 1H), 2.24-2.15 (m, 4H), 2.25-2.15 (m, 1H), 1.76-1.71 (m, 1H) , 1.59-1.54 (m, 1H), 1.30 (d, J = 6.4 Hz, 3H), 1.26 (s, 3H), 1.24 (s, 3H), 1.06 (s, 3H). | LCMS (ES, m/z): 528.25 [M + H]+, Rt 0.645 min. |
| 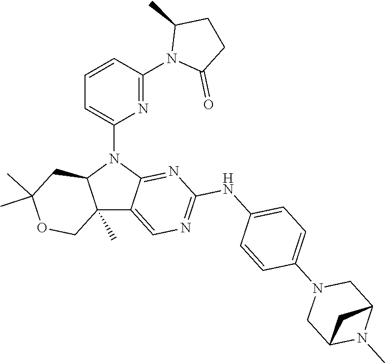 | 748 | 1H NMR (400 MHz, DMSO-d6) δ 9.01 (s, 1H), 8.43 (d, J = 8.0 Hz, 1H), 7.98 (s, 1H), 7.85-7.73 (m, 2H), 7.52 (d, J = 8.4 Hz, 2H), 6.74-6.66 (m, 2H), 4.90-4.79 (m, 1H), 4.67-4.59 (m, 1H), 3.87-3.79 (m, 1H), 3.71-3.64 (m, 1H), 3.61-3.55 (m, 2H), 3.46-3.39 (m, 2H), 3.30-3.23 (m, 2H), 2.86-2.73 (m, 1H), 2.50-2.38 (m, 2H), 2.33-2.14 (m, 2H), 2.00 (s, 3H), 1.79-1.68 (m, 1H), 1.60-1.50 (m, 2H), 1.29 (d, J = 6.4 Hz, 3H), 1.25 (s, 6H), 1.06 (s, 3H). | LCMS (ES, m/z): 595.35 [M + H]+, Rt 0.725 min. |
| 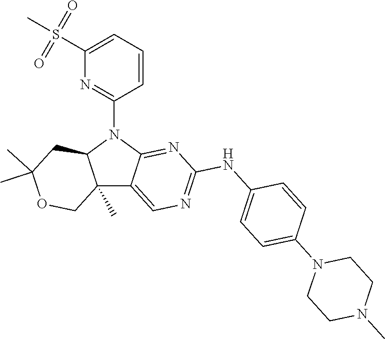 | 749 | 1H NMR (400 MHz, DMSO-d6) δ 9.22 (s, 1H), 8.97 (d, J = 8.8 Hz, 1H), 8.15-8.07 (m, 2H), 7.62 (d, J = 7.6 Hz, 1H), 7.53 (d, J = 8.4 Hz, 2H), 6.92 (d, J = 8.4 Hz, 2H), 4.68-4.60 (m, 1H), 3.83-3.65 (m, 2H), 3.34-3.31 (m, 4H), 3.12-3.05 (m, 4H), 2.47 (s, 3H), 2.31-2.19 (m, 4H), 1.70- 1.60 (m, 1H), 1.32 (s, 3H), 1.24 (s, 3H), 1.03 (s, 3H). | LCMS (ES, m/z): 564.30 [M + H]+, Rt 0.588 min. |
| 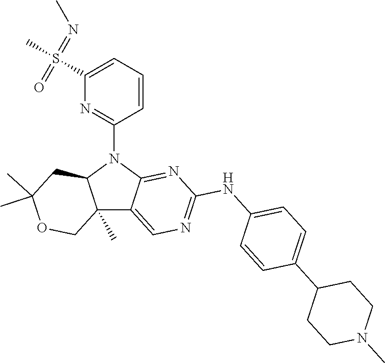 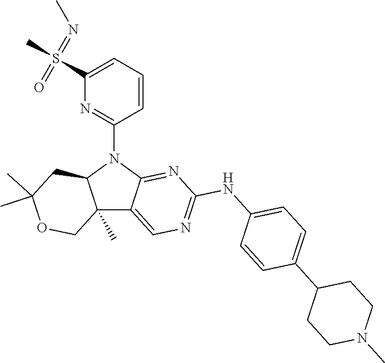 | 751, 752 | 1H NMR (400 MHz, DMSO-d6) δ 9.37 (s, 1H), 8.87 (d, J = 8.4 Hz, 1H), 8.13-8.02 (m, 2H), 7.68-7.55 (m, 3H), 7.17 (d, J = 8.4 Hz, 2H), 4.68-4.55 (m, 1H), 3.82- 3.65 (m, 2H), 3.20 (s, 3H), 2.92-2.82 (m, 2H), 2.51(s, 3H), 2.48-2.37 (m, 1H), 2.29-2.18 (m, 4H), 2.02-1.92 (m, 2H), 1.85-1.59 (m, 5H), 1.32 (s, 3H), 1.21 (s, 3H), 1.02 (s, 3H). 1H NMR (400 MHz, DMSO-d6) δ 9.37 (s, 1H), 8.89 (d, J = 8.4 Hz, 1H), 8.15-8.02 (m, 2H), 7.68-7.55 (m, 3H), 7.17 (d, J = 8.4 Hz, 2H), 4.68-4.57 (m, 1H), 3.89- 3.81 (m, 1H), 3.72-3.63 (m, 1H), 3.30 (s, 3H), 2.92-2.85 (m, 2H), 2.52 (s, 3H), 2.48-2.37 (m, 1H), 2.24-2.16 (m, 4H), 2.02-1.90 (m, 2H), 1.78-1.61 (m, 5H), 1.32 (s, 3H), 1.22 (s, 3H), 1.03 (s, 3H). | LCMS (ES, m/z): 576.30 [M + H]+, Rt 1.058 min. LCMS (ES, m/z): 576.30 [M + H]+, Rt 1.048 min. |
| 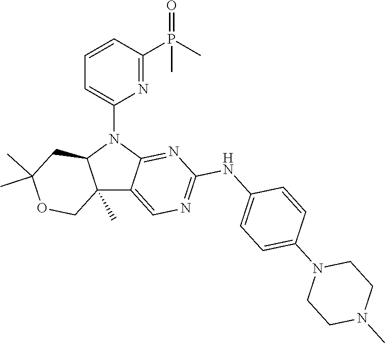 | 753 | 1H NMR (400 MHz, DMSO-d6) δ 9.18 (s, 1H), 8.82 (d, J = 8.8 Hz, 1H), 8.03 (s, 1H), 8.00-7.91 (m, 1H), 7.61-7.51 (m, 3H), 6.95-6.86 (m, 2H), 4.66-4.58 (m, 1H), 3.83-3.64 (m, 2H), 3.11-3.04 (m, 4H), 2.49-2.42 (m, 4H), 2.22 (s, 3H), 2.20-2.17 (m, 1H), 1.70 (s, 3H), 1.67 (s, 3H), 1.63-1.53 (m, 1H), 1.30 (s, 3H), 1.24 (s, 3H), 1.05 (s, 3H). | LCMS (ES, m/z): 562.30 [M + H]+, Rt 0.575 min. |
| 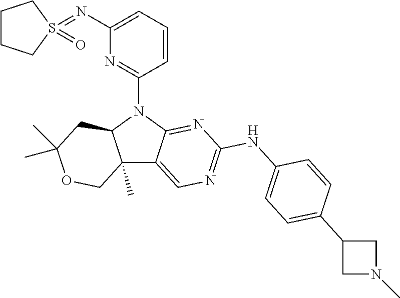 [SMILES: CN1CC(c2ccc(Nc3ncc4c(n3)N(c3cccc(N=S5(=O)CCCC5)n3)[C@@H]3CC(C)(C)OC[C@]43C)cc2)C1] | 754 | 1H NMR (400 MHz, DMSO-d6) δ 9.25 (s, 1H), 8.19-8.11 (m, 1H), 8.02 (s, 1H), 7.77-7.65 (m, 2H), 7.57 (t, J = 8.0 Hz, 1H), 7.23 (d, J = 8.4 Hz, 2H), 6.39 (d, J = 7.8 Hz, 1H), 4.72-4.52 (m, 1H), 3.90- 3.80 (m, 1H), 3.75-3.58 (m, 4H), 3.58- 3.42 (m, 2H), 3.33-3.21 (m, 2H), 3.15- 3.05 (m, 2H), 2.41-2.33 (m, 1H), 2.25- 2.02 (m, 7H), 1.62-1.45 (m, 1H), 1.27 (s, 3H), 1.22 (s, 3H), 1.02 (s, 3H). | LCMS (ES, m/z): 574 [M + H]+, Rt 0.616 min. |
| 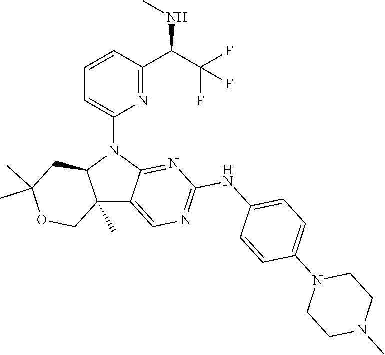 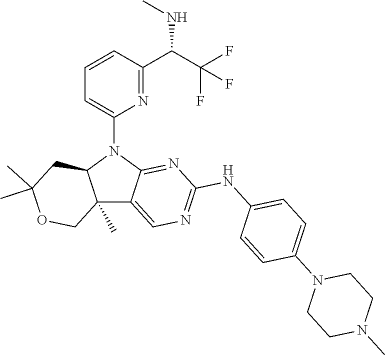 | 755, 756 | 1H NMR (300 MHz, DMSO-d6) δ 9.13 (s, 1H), 8.66 (d, J = 8.4 Hz, 1H), 8.00 (s, 1H), 7.86 (t, J = 7.8 Hz, 1H), 7.55 (d, J = 8.1 Hz, 2H), 7.22 (d, J = 7.2 Hz, 1H), 6.91 (d, J = 9.0 Hz, 2H), 4.64-4.59 (m, 1H), 4.36-4.30 (m, 1H), 3.77-3.63 (m, 2H), 3.08-3.05 (m, 4H), 2.50-2.49 (m, 1H), 2.47-2.44 (m, 4H), 2.30 (s, 3H), 2.22 (s, 3H), 2.17-2.16 (m, 1H), 1.67-1.64 (m, 1H), 1.35 (s, 3H), 1.23 (s, 3H), 1.14 (s, 3H). 1H NMR (300 MHz, DMSO-d6) δ 9.13 (s, 1H), 8.67 (d, J = 8.4 Hz, 1H), 8.00 (s, 1H), 7.85 (t, J = 7.5 Hz, 1H), 7.55 (d, J = 8.1 Hz, 2H), 7.17 (d, J = 7.2 Hz, 1H), 6.91 (d, J = 9.0 Hz, 2H), 4.72-4.68 (m, 1H), 4.36-4.31 (m, 1H), 3.77-3.62 (m, 2H), 3.08-3.05 (m, 4H), 2.50-2.49 (m, 1H), 2.47-2.44 (m, 4H), 2.30 (s, 3H), 2.22- 2.14 (m, 4H), 1.64-1.59 (m, 1H), 1.29 (s, 3H), 1.20 (s, 3H), 1.02 (s, 3H). | LCMS (ES, m/z): 597.20 [M + H]+, Rt 1.018 min. LCMS (ES, m/z): 597.00 [M + H]+, Rt 1.078 min. |
| 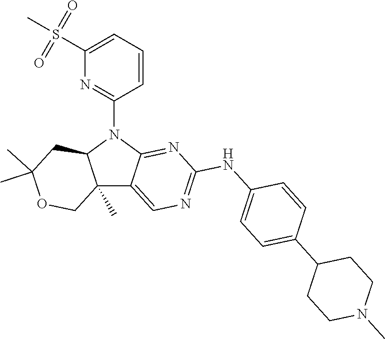 | 757 | 1H NMR (400 MHz, DMSO-d6) δ 9.40 (s, 1H), 8.97 (d, J = 8.4 Hz, 1H), 8.15-8.06 (m, 2H), 7.66-7.58 (m, 3H), 7.17 (d, J = 8.4 Hz, 2H), 4.82-4.50 (m, 1H), 3.83- 3.76 (m, 1H), 3.72-3.65 (m, 1H), 3.33 (s, 3H), 2.90-2.83 (m, 2H), 2.45-2.34 (m, 1H), 2.26-2.14 (m, 4H), 2.01-1.91 (m, 2H), 1.76-1.60 (m, 5H), 1.32 (s, 3H), 1.23 (s, 3H), 1.03 (s, 3H). | LCMS (ES, m/z): 563.20 [M + H]+, Rt 0.600 min. |
| 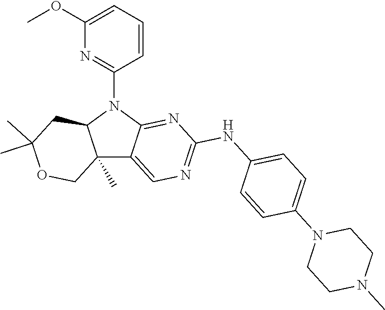 | 758 | 1H NMR (400 MHz, DMSO-d6) δ 9.10 (s, 1H), 8.24 (d, J = 8.0 Hz, 1H), 7.98 (s, 1H), 7.69 (t, J = 8.0 Hz, 1H), 7.55 (d, J = 8.8 Hz, 2H), 6.93-6.85 (m, 2H), 6.44 (d, J = 8.0 Hz, 1H), 4.67-4.59 (m, 1H), 3.87 (s, 3H), 3.82-3.74 (m, 1H), 3.70-3.63 (m, 1H), 3.10-3.03 (m, 4H), 2.49-2.42 (m, 4H), 2.35-2.26 (m, 1H), 2.22 (s, 3H), 1.64-1.53 (m, 1H), 1.29 (s, 3H), 1.24 (s, 3H), 1.06 (s, 3H). | LCMS (ES, m/z): 516.30 [M + H]+, Rt 0.930 min. |
| 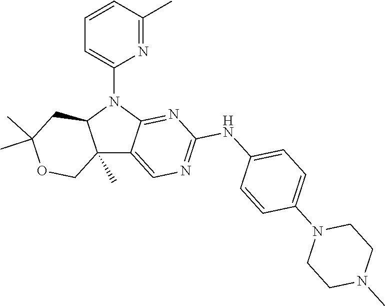 | 759 | 1H NMR (400 MHz, DMSO-d6) δ 9.07 (s, 1H), 8.43 (d, J = 8.4 Hz, 1H), 7.98 (s, 1H), 7.67 (t, J = 8.0 Hz, 1H), 7.55 (d, J = 8.4 Hz, 2H), 6.93-6.85 (m, 3H), 4.71- 4.63 (m, 1H), 3.80-3.61 (m, 2H), 3.10- 3.03 (m, 4H), 2.54-2.49 (m, 2H), 2.48- 2.40 (m, 6H), 2.22 (s, 3H), 1.68-1.58 (m, 1H), 1.26 (s, 3H), 1.21 (s, 3H), 1.00 (s, 3H). | LCMS (ES, m/z): 500.30 [M + H]+, Rt 0.675 min. |
| 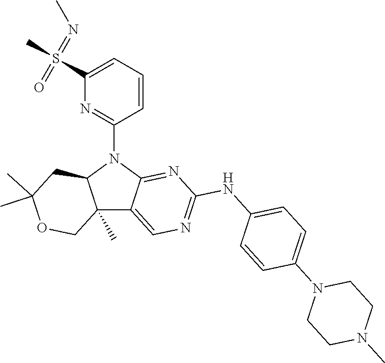 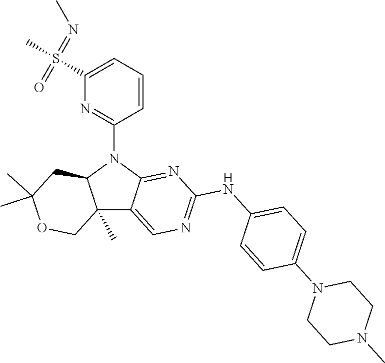 | 765, 766 | 1H NMR (400 MHz, DMSO-d6) δ 9.26 (s, 1H), 8.89 (d, J = 8.4 Hz, 1H), 8.08 (m, 2H), 7.58 (d, J = 7.2 Hz, 3H), 6.97 (d, J = 9.2 Hz, 2H), 4.64-4.60 (m, 1H), 3.81- 3.79 (m, 1H), 3.69-3.61 (m, 1H), 3.27 (s, 3H), 3.21-3.00 (m, 3H), 2.98-2.68 (m, 4H), 2.63-2.51 (m, 4H), 2.50 (s, 3H), 2.22-2.17 (m, 1H), 1.64-1.58 (m, 1H), 1.30 (s, 3H), 1.21 (s, 3H), 0.99 (s, 3H). 1H NMR (400 MHz, DMSO-d6) δ 9.20 (s, 1H), 8.86 (d, J = 8.4 Hz, 1H), 8.09-8.02 (m, 2H), 7.62-7.49 (m, 3H), 6.91 (d, J = 8.0 Hz, 2H), 4.59 (d, J = 8.4 Hz, 1H), 3.80-3.60 (m, 2H), 3.21 (s, 3H), 3.07 (t, J = 4.8 Hz, 4H), 2.50-2.48 (m, 3H), 2.48- 2.42 (m, 4H), 2.25-2.18 (m, 4H), 1.79- 1.72 (m, 1H), 1.31 (s, 3H), 1.21 (s, 3H), 0.99 (s, 3H). | LCMS (ES, m/z): 577.20 [M + H]+, Rt 0.928 min. LCMS (ES, m/z): 577.20 [M + H]+, Rt 0.958 min. |
| 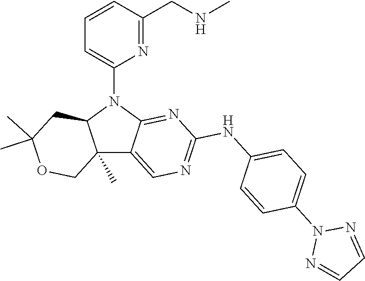 [SMILES: CNCc1cccc(N2c3nc(Nc4ccc(-n5nccn5)cc4)ncc3[C@@]3(C)COC(C)(C)C[C@@H]23)n1] | 767 | 1H NMR (300 MHz, DMSO-d6) δ 9.64 (s, 1H), 8.49 (d, J = 8.4 Hz, 1H), 8.09 (s, 1H), 8.07 (s, 2H), 8.01-7.88 (m, 4H), 7.80 (t, J = 9.0 Hz, 1H), 7.08 (d, J = 7.5 Hz, 1H), 4.77-4.67 (m, 1H), 3.84-3.76 (m, 1H), 3.72 (s, 2H), 3.71-3.64 (m, 1H), 2.35 (s, 3H), 2.27-2.15 (m, 1H), 1.73- 1.60 (m, 1H), 1.30 (s, 3H), 1.23 (s, 3H), 1.02 (s, 3H). | LCMS (ES, m/z): 498.20 [M + H]+, Rt 0.667 min. |
| 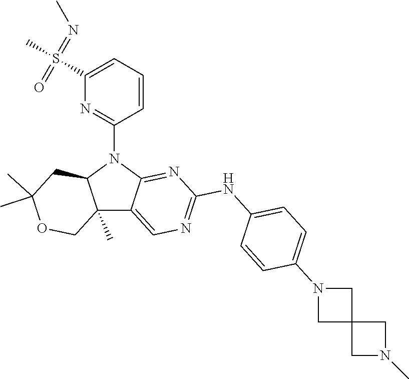 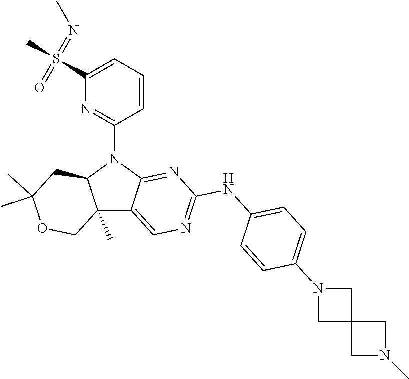 | 770, 771 | 1H NMR (400 MHz, DMSO-d6) δ 9.10 (s, 1H), 8.85 (d, J = 8.8 Hz, 1H), 8.08-7.95 (m, 2H), 7.59 (d, J = 7.2 Hz, 1H), 7.45 (d, J = 8.4 Hz, 2H), 6.41 (d, J = 8.0 Hz, 2H), 4.61-4.56 (m, 1H), 3.87-3.69 (m, 5H), 3.69-3.58 (m, 1H), 3.32-3.30 (m, 3H), 3.30-3.25 (s, 4H), 3.21 (s, 3H), 2.25-2.16 (m, 4H), 1.79-1.70 (m, 1H), 1.25 (d, J = 37.4 Hz, 6H), 0.99 (s, 3H). 1H NMR (400 MHz, DMSO-d6) δ 9.09 (s, 1H), 8.88 (d, J = 8.4 Hz, 1H), 8.04-7.99 (m, 2H), 7.56 (d, J = 7.2 Hz, 1H), 7.46 (d, J = 8.4 Hz, 2H), 6.43 (d, J = 8.8 Hz, 2H), 4.62-4.58 (m, 1H), 4.13-3.91 (m, 1H), 3.82 (s, 3H), 3.78-3.65 (m, 2H), 3.38 (s, 3H), 3.32 (s, 4H), 3.26 (s, 3H), 2.26 (s, 3H), 2.22-2.17 (m, 1H), 1.63-1.57 (m, 1H), 1.29 (s, 3H), 1.20 (s, 3H), 1.02 (s, 3H) | LCMS (ES, m/z): 589.25 [M + H]+, Rt 0.525 min. LCMS (ES, m/z): 589.30 [M + H]+, Rt 0.575 min. |
| 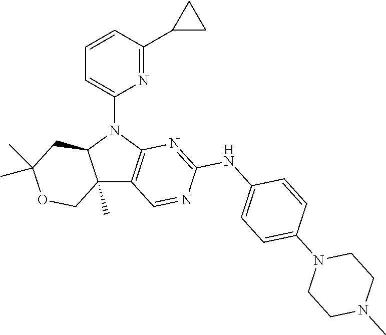 | 774 | 1H NMR (300 MHz, DMSO-d6) δ 9.06 (s, 1H), 8.41 (d, J = 8.4 Hz, 1H), 7.94 (s, 1H), 7.64-7.52 (m, 3H), 6.99-6.86 (m, 3H), 4.55-4.50 (m, 1H), 3.76 (d, J = 12 Hz, 1H), 3.66 (d, J = 11.7 Hz, 1H), 3.07- 3.04 (m, 4H), 2.47-2.44 (m, 4H), 2.22- 2.13 (m, 4H), 2.07-2.04 (m, 1H), 1.55- 1.47 (m, 1H), 1.26 (s, 3H), 1.24 (s, 3H), 1.04-0.82 (m, 7H). | LCMS (ES, m/z): 526.20 [M + H]+, Rt 0.725 min. |
| 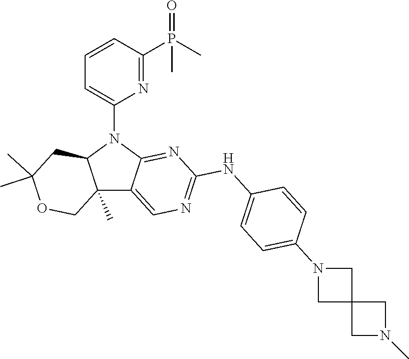 | 775 | 1H NMR (300 MHz, DMSO-d6) δ 9.10 (s, 1H), 8.86-8.76 (m, 1H), 8.01 (s, 1H), 7.98-7.86 (m, 1H), 7.59-7.53 (m, 1H), 7.46 (d, J = 8.4 Hz, 2H), 6.46-6.36 (m, 2H), 4.67-4.56 (m, 1H), 3.85-3.74 (m, 4H), 3.72-3.63 (m, 1H), 3.25 (s, 4H), 2.19 (s, 4H), 1.70 (s, 3H), 1.66 (s, 3H), 1.63-1.50 (m, 2H), 1.29 (s, 3H), 1.24 (s, 3H), 1.05 (s, 3H). | LCMS (ES, m/z): 574.30 [M + H]+, Rt 0.567 min. |
| 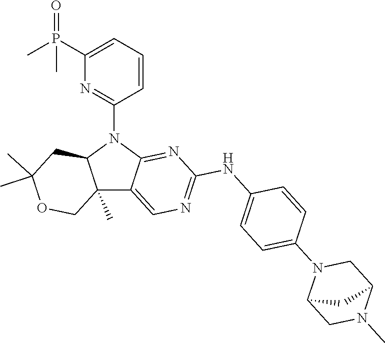 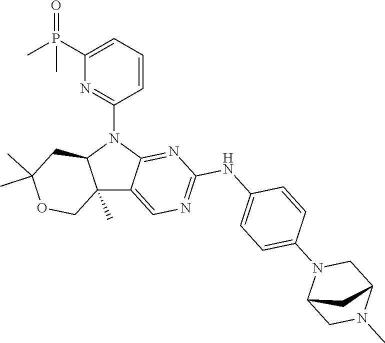 | 777, 778 | 1H NMR (400 MHz, DMSO-d6) δ 9.01 (s, 1H), 8.82 (d, J = 8 Hz, 1H), 7.99 (s, 1H), 7.94-7.87 (m, 1H), 7.55 (t, J = 5.6 Hz, 1H), 7.43 (d, J = 8.8 Hz, 2H), 6.56 (d, J = 9.2 Hz, 2H), 4.62-4.59 (m, 1H), 4.24 (s, 1H), 3.79-3.65 (m, 2H), 3.39 (s, 1H), 3.30 (s, 1H), 3.12 (d, J = 9.2 Hz, 1H), 2.77-2.75 (m, 1H), 2.49 (s, 1H), 2.22- 2.17 (m, 4H), 1.86 (d, J = 8.8 Hz, 1H), 1.77 (d, J = 8.8 Hz, 1H), 1.69-1.66 (m, 6H), 1.59-1.54 (m, 1H), 1.29 (s, 3H), 1.23 (s, 3H), 1.04 (s, 3H). 1H NMR (400 MHz, DMSO-d6) δ 9.01 (s, 1H), 8.81 (d, J = 8.8 Hz, 1H), 8.04-7.83 (m, 2H), 7.58-7.49 (m, 1H), 7.42 (d, J = 8.4 Hz, 2H), 6.55 (d, J = 8.8 Hz, 2H), 4.65-4.55 (m, 1H), 4.24 (s, 1H), 3.82- 3.61 (m, 2H), 3.41-3.36 (m, 1H), 3.31- 3.27 (m, 1H), 3.11 (d, J = 8.8 Hz, 1H), 2.80-2.71 (m, 1H), 2.48-2.44 (m, 1H), 2.28-2.13 (m, 4H), 1.89-1.80 (m, 1H), 1.79-1.72 (m, 1H), 1.67 (s, 3H), 1.62 (s, 3H), 1.59-1.51 (m, 1H), 1.30 (s, 3H), 1.23 (s, 3H), 1.04 (s, 3H). | LCMS (ES, m/z): 574.25 [M + H]+, Rt 1.140 min. LCMS (ES, m/z): 574.25 [M + H]+, Rt 0.818 min. |
| 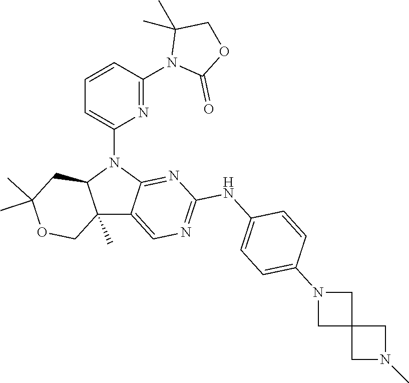 | 785 | 1H NMR (400 MHz, DMSO-d6) δ 8.96 (s, 1H), 8.35 (d, J = 8.4 Hz, 1H), 7.95 (s, 1H), 7.73 (t, J = 8.0 Hz, 1H), 7.39 (d, J = 8.4 Hz, 2H), 7.22 (d, J = 7.6 Hz, 1H), 6.36-6.29 (m, 2H), 4.55-4.47 (m, 1H), 4.15-4.05 (m, 2H), 3.82-3.71 (m, 5H), 3.64-3.56 (m, 1H), 3.19 (s, 4H), 2.12 (s, 3H), 2.08-2.02 (m, 1H), 1.55 (s, 3H), 1.52 (s, 3H), 1.50-1.39 (m, 1H), 1.16 (s, 3H), 1.12 (s, 3H), 0.96 (s, 3H). | LCMS (ES, m/z): 611.30 [M + H]+, Rt 0.658 min. |
| 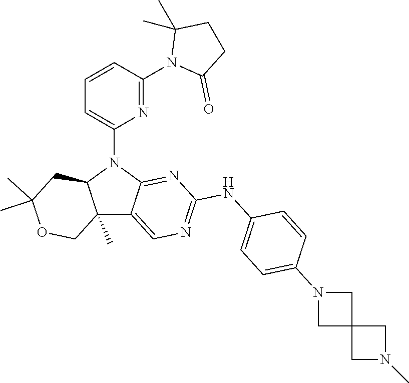 | 786 | 1H NMR (400 MHz, DMSO-d6) δ 9.02 (s, 1H), 8.48-8.41 (m, 1H), 8.00 (s, 1H), 7.78 (t, J = 8.0 Hz, 1H), 7.62-7.30 (m, 2H), 7.17 (d, J = 7.8 Hz, 1H), 6.39 (d, J = 8.8 Hz, 2H), 4.70-4.45 (m, 1H), 3.99- 3.75 (m, 5H), 3.70-3.50 (m, 1H), 3.25 (s, 4H), 2.59-2.51 (m, 2H), 2.21-2.08 (m, 4H), 2.09-1.85 (m, 2H), 1.62-1.45 (m, 7H), 1.23 (s, 3H), 1.19 (s, 3H), 1.02 (s, 3H). | LCMS (ES, m/z): 609.35 [M + H]+, Rt 0.667 min. |
| 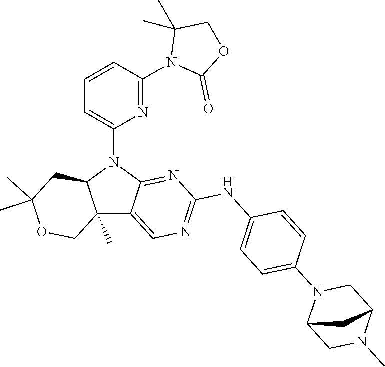 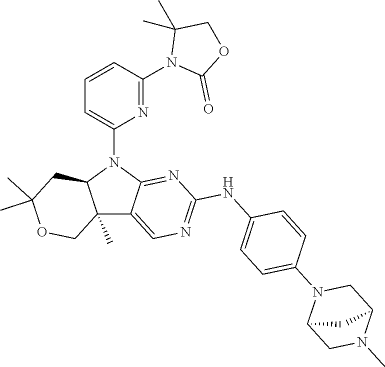 | 795, 796 | 1H NMR (300 MHz, DMSO-d6) δ 8.97 (s, 1H), 8.43 (d, J = 3.9 Hz, 1H), 8.01 (s, 1H), 7.79 (d, J = 8.4 Hz, 1H), 7.51-7.42 (m, 2H), 7.30 (d, J = 8.4 Hz, 1H), 6.62- 6.53 (m, 2H), 4.63-4.48 (m, 1H), 4.23- 4.17 (m, 3H), 3.88-3.84 (m, 1H), 3.69- 3.65 (m, 1H), 3.43-3.35 (m, 1H), 3.15- 3.08 (m, 1H), 2.78-2.74 (m, 1H), 2.55- 2.45 (m, 2H), 2.24 (s, 3H), 2.14-2.09 (m, 1H), 1.88-1.80 (m, 1H), 1.79-1.70 (m, 1H), 1.60-1.46 (m, 7H), 1.24 (s, 3H), 1.18 (s, 3H), 1.03 (s, 3H). 1H NMR (400 MHz, DMSO-d6) δ 8.88 (s, 1H), 8.36 (d, J = 8.4 Hz, 1H), 7.94 (s, 1H), 7.73 (t, J = 8.0 Hz, 1H), 7.36 (d, J = 8.8 Hz, 2H), 7.21 (d, J = 7.6 Hz, 1H), 6.47 (d, J = 8.8 Hz, 2H), 4.55-4.47 (m, 1H), 4.20-4.05 (m, 3H), 3.82-3.75 (m, 1H), 3.64-3.56 (m, 1H), 3.36-3.29 (m, 2H), 3.08-3.01 (m, 1H), 2.70-2.64 (m, 1H), 2.43-2.39 (m, 1H), 2.17 (s, 3H), 2.09- 2.00 (m, 1H), 1.81-1.74 (m, 1H), 1.71- 1.64 (m, 1H), 1.55 (s, 3H), 1.52 (s, 3H), 1.50-1.40 (m, 1H), 1.17 (s, 3H), 1.12 (s, 3H), 0.96 (s, 3H). | LCMS (ES, m/z): 611.35 [M + H]+, Rt 0.675 min. LCMS (ES, m/z): 611.35 [M + H]+, Rt 0.683 min. |
| 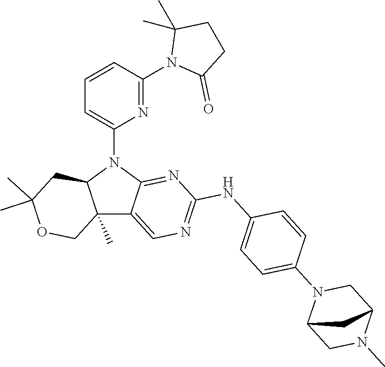 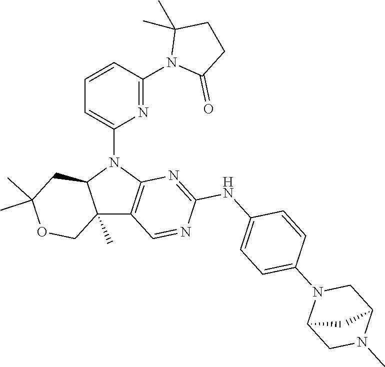 | 797, 798 | 1HNMR (300 MHz, DMSO-d6)δ8.96 (s, 1H), 8.45 (d, J = 8.4 Hz, 1H), 7.99 (s, 1H), 7.76 (t, J = 8.1 Hz, 1H), 7.43 (d, J = 8.7 Hz, 2H), 7.16 (d, J = 7.5 Hz, 1H), 6.54 (d, J = 9.0 Hz, 2H), 4.59-4.54 (m, 1H), 4.23 (s, 1H), 3.86-3.82 (m, 1H), 3.68- 3.64 (m, 1H), 3.37-3.34 (m, 1H), 3.29- 3.26 (m, 1H), 3.12-3.09 (m, 1H), 2.77- 2.73 (m, 1H), 2.58-2.56 (m, 2H), 2.53- 2.50 (m, 1H), 2.23 (s, 3H), 2.16-2.09 (m, 1H), 1.94 (t, J = 7.8 Hz, 2H), 1.85-1.73 (m, 2H), 1.55-1.49 (m, 7H), 1.23 (s, 3H), 1.18 (s, 3H), 1.02 (s, 3H). 1H NMR (300 MHz, DMSO-d6) δ 8.96 (s, 1H), 8.46 (d, J = 8.1 Hz, 1H), 8.00 (s, 1H), 7.77 (t, J = 8.1 Hz, 1H), 7.43 (d, J = 8.7 Hz, 2H), 7.16 (d, J = 7.8 Hz, 1H), 6.54 (d, J = 8.7 Hz, 2H), 4.68-4.43 (m, 1H), 4.23 (s, 1H), 4.01-3.79 (m, 1H), 3.75- 3.55 (m, 1H), 3.38 (s, 1H), 3.20-2.99 (m, 1H),2.90-2.78 (m, 1H), 2.85-2.62 (m, 1H),2.62-2.60 (m, 2H), 2.55-2.52 (m, 1H), 2.24 (s, 3H), 2.18-2.02 (m, 1H), 1.95 (t,J = 7.8 Hz, 2H), 1.88-1.63 (m, 2H), 1.56-1.42 (m, 7H), 1.23 (s, 3H), 1.19 (s, 3H), 1.03 (s, 3H). | LCMS (ES, m/z): 609.35 [M + H]+, Rt 0.683 min. LCMS (ES, m/z): 609.35 [M + H]+, Rt 0.667 min. |
| 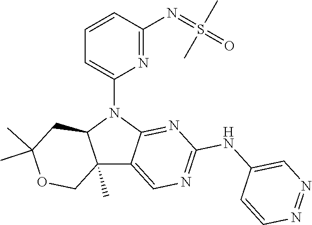 [SMILES: CC1(C)C[C@H]2N(c3cccc(N=S(C)(C)=O)n3)c3nc(Nc4ccnnc4)ncc3[C@@]2(C)CO1] | 799 | 1H NMR (300 MHz, DMSO-d6) δ 10.07 (s, 1H), 9.43-9.42 (m, 1H), 8.90 (d, J = 6.0 Hz, 1H), 8.18-8.16 (m, 2H), 8.03 (d, J = 8.1 Hz, 1H), 7.5-7.60 (m, 1H), 6.38 (d, J = 7.8 Hz, 1H), 4.62-4.57 (m, 1H), 3.89- 3.85 (m, 1H), 3.70-3.66 (m, 1H), 3.49 (s, 3H), 3.31 (s, 3H), 2.46-2.42 (m, 1H), 1.61-1.53 (m, 1H), 1.30 (s, 3H), 1.23 (s, 3H), 1.02 (s, 3H) | LCMS (ES, m/z): 481.20 [M + H]+, Rt 0.575 min. |
| 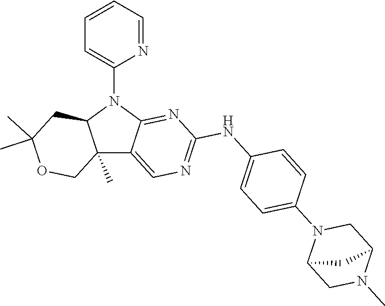 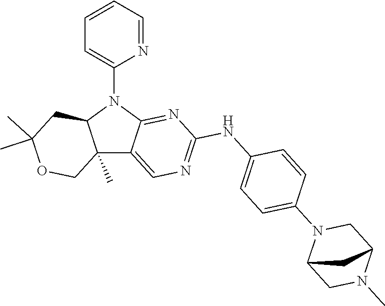 | 800, 801 | 1H NMR (400 MHz, DMSO-d6) δ 8.95 (s, 1H), 8.64 (d, J = 8.8 Hz, 1H), 8.41-8.29 (m, 1H), 7.98 (s, 1H), 7.81-7.69 (m, 1H), 7.43 (d, J = 8.8 Hz, 2H), 7.09-6.92 (m, 1H), 6.54 (d, J = 8.8 Hz, 2H), 4.73-4.59 (m, 1H), 4.24 (s, 1H), 3.90-3.75 (m, 1H), 3.74-3.60 (m, 1H), 3.39-3.37 (m, 1H), 3.28-3.25 (m, 1H), 3.18-3.11 (m, 1H), 2.78-2.68 (m, 1H), 2.45-2.42 (m, 1H), 2.24 (s, 3H), 2.19-2.11 (m, 1H), 1.87- 1.75 (m, 2H), 1.63-1.54 (m, 1H), 1.23 (s, 3H), 1.20 (s, 3H) 1.00 (s, 3H). 1H NMR (400 MHz, DMSO-d6) δ 8.94 (s, 1H), 8.63 (d, J = 8.4 Hz, 1H), 8.39-8.28 (m, 1H), 7.98 (s, 1H), 7.78-7.65 (m, 1H), 7.43 (d, J = 8.8 Hz, 2H), 7.08-6.92 (m, 1H), 6.54 (d, J = 8.8 Hz, 2H), 4.73-4.45 (m, 1H), 4.23 (s, 1H), 3.85-3.75 (m, 1H), 3.74-3.61 (m, 1H), 3.39-3.31 (m, 1H), 3.30-3.28 (m, 1H), 3.18-3.10 (m, 1H), 2.80-2.70 (m, 1H), 2.48-2.43 (m, 1H), 2.24 (s, 3H), 2.17-2.08 (m, 1H), 1.82- 1.69 (m, 2H), 1.65-1.48 (m, 1H), 1.22 (s, 3H), 1.19(s, 3H), 1.00 (s, 3H). | LCMS (ES, m/z): 498.30 [M + H]+, Rt 0.622 min. LCMS (ES, m/z): 498.30 [M + H]+, Rt 0.622 min. |
| 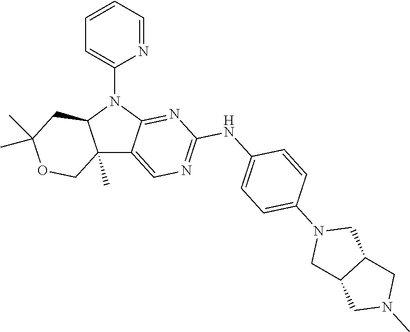 | 803 | 1H NMR (400 MHz, DMSO-d6) δ 9.00 (s, 1H), 8.64 (d, J = 8.4 Hz, 1H), 8.48-8.38 (m, 1H),7.99 (s, 1H), 7.70 (t, J = 6.8 Hz, 1H), 7.48 (d, J = 8.8 Hz, 2H), 7.07-6.98 (m, 1H), 6.62 (d, J = 8.8 Hz, 2H), 4.75- 4.51 (m, 1H), 3.89-3.72 (m, 1H), 3.72- 3.65 (m, 1H), 3.31-3.25(m, 2H), 3.07- 3.00 (m, 2H), 2.90-2.81 (m, 2H), 2.63- 2.55 (m, 2H), 2.41-2.31 (m, 2H), 2.23 (s, 3H), 2.21-2.12 (m, 1H), 1.69-1.55 (m, 1H), 1.23 (s, 3H), 1.20 (s, 3H), 1.00 (s, 3H) | LCMS (ES, m/z): 512.35 [M + H]+, Rt 0.630 min. |
| 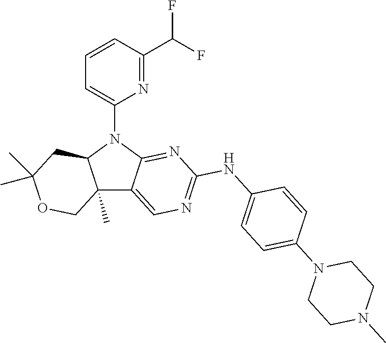 | 807 | 1H NMR (400 MHz, DMSO-d6) δ 9.17 (s, 1H), 8.80 (d, J = 8.4 Hz, 1H), 8.05 (s, 1H), 7.97 (t,J = 8.4 Hz, 1H), 7.53 (d, J = 8.8 Hz, 2H), 7.31 (d, J = 7.2 Hz, 1H), 7.04-6.74 (m, 3H), 4.66-4.58 (m, 1H), 3.80-3.73 (m, 1H), 3.69-3.62 (m, 1H), 3.11-3.04 (m, 4H), 2.49-2.42 (m, 4H), 2.22 (s, 3H), 2.23-2.15 (m, 1H), 1.72- 1.62 (m, 1H), 1.28 (s, 3H), 1.21 (s, 3H), 0.99 (s, 3H). | LCMS (ES, m/z): 536.30 [M + H]+, Rt 0.675 min. |
| 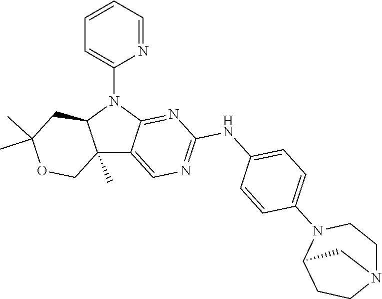 [SMILES: CC1(C)C[C@H]2N(c3ccccn3)c3nc(Nc4ccc(N5CCN6CC[C@@H]5C6)cc4)ncc3[C@@]2(C)CO1] 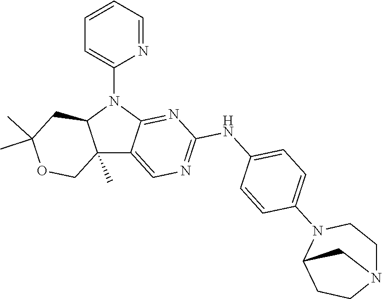 [SMILES: CC1(C)C[C@H]2N(c3ccccn3)c3nc(Nc4ccc(N5CCN6CC[C@H]5C6)cc4)ncc3[C@@]2(C)CO1] | 808, 809 | 1H NMR (300 MHz, DMSO-d6) δ 9.06 (s, 1H), 8.62 (d, J = 8.7 Hz, 1H), 8.40-8.32 (m, 1H), 8.00 (s, 1H), 7.83-7.72 (m, 1H), 7.52 (d, J = 8.7 Hz, 2H), 7.08-6.98 (m, 1H), 6.85 (d, J = 9.0 Hz, 2H), 4.72-4.61 (m, 1H), 4.22 (t, J = 3.9 Hz, 1H), 3.85- 3.61 (m, 2H), 3.27-3.21 (m, 1H), 3.09- 2.96 (m, 3H), 2.90-2.75 (m, 3H), 2.61- 2.54 (m, 1H), 2.21-2.11 (m, 1H), 1.77- 1.55 (m, 3H), 1.24 (s, 3H), 1.20 (s, 3H), 0.99 (s, 3H). 1H NMR (300 MHz, DMSO-d6) δ 9.06 (s, 1H), 8.63 (d, J = 8.4 Hz, 1H), 8.36 (d, J = 4.8 Hz, 1H), 8.00 (s, 1H), 7.85-7.69 (m, 1H), 7.53 (d, J = 9.0 Hz, 2H), 7.08-6.98 (m, 1H), 6.86 (d, J = 9.0 Hz, 2H), 4.73- 4.62 (m, 1H), 4.28-4.21 (m, 1H), 3.81- 3.62 (m, 2H), 3.21-2.98 (m, 4H), 2.93- 2.78 (m, 3H), 2.65-2.55 (m, 1H), 2.22- 2.12 (m, 1H), 2.07-1.94 (m, 1H), 1.76- 1.56 (m, 2H), 1.23 (s, 3H), 1.20 (s, 3H), 0.99 (s, 3H). | LCMS (ES, m/z): 498.15 [M + H]+, Rt 0.613 min. LCMS (ES, m/z): 498.15 [M + H]+, Rt 0.617 min. |
| 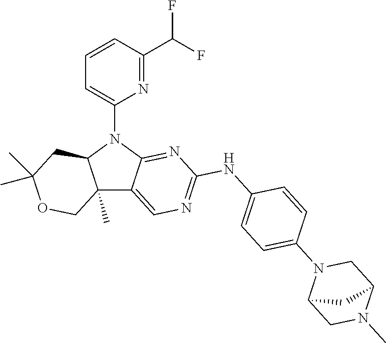 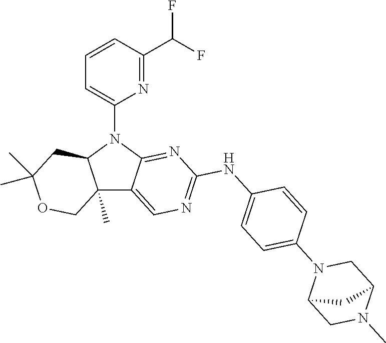 | 813, 814 | 1H NMR (400 MHz, DMSO-d6) δ 9.01 (s, 1H), 8.80 (d, J = 8.4 Hz, 1H), 8.02 (s, 1H), 7.93 (t, J = 7.6 Hz, 1H), 7.42 (d, J = 8.8 Hz, 2H), 7.29 (d, J = 7.2 Hz, 1H), 7.09 -6.70 (m, 1H), 6.55 (d, J = 8.8 Hz, 2H), 4.68-4.50 (m, 1H), 4.26-4.21 (m, 1H), 3.80-3.72 (m, 1H), 3.71-3.58 (m, 1H), 3.41-3.38 (m, 1H), 3.32-3.29 (m, 2H), 3.09-3.15 (m, 1H), 2.80-2.71 (m, 1H), 2.31-2.11 (m, 4H), 1.91-1.81 (m, 1H), 1.80-1.71 (m, 1H), 1.70-1.61 (m, 1H), 1.27 (s, 3H), 1.20 (s, 3H), 1.00 (s, 3H). 1H NMR (400 MHz, DMSO-d6) δ 9.01 (s, 1H), 8.80 (d, J = 8.8 Hz, 1H), 8.02 (s, 1H), 7.92 (t, J = 8.0 Hz, 1H), 7.42 (d, J = 8.8 Hz, 2H), 7.29 (d, J = 7.2 Hz, 1H), 7.05-6.73 (m, 1H), 6.54 (d, J = 9.2 Hz, 2H), 4.66-4.57 (m, 1H), 4.23 (s, 1H), 3.81-3.66 (m, 2H), 3.46-3.37 (m, 2H), 3.33-3.28 (m, 1H), 3.11 (d, J = 9.2 Hz, 1H), 2.80-2.73 (m, 1H), 2.24 (s, 3H), 2.23-2.17 (m, 1H), 1.89-1.71 (m, 2H), 1.70-1.61 (m, 1H), 1.26 (s, 3H), 1.20 (s, 3H), 0.99 (s, 3H). | LCMS (ES, m/z): 548.30 [M + H]+, Rt 0.692 min. LCMS (ES, m/z): 548.25 [M + H]+, Rt 0.683 min. |
| 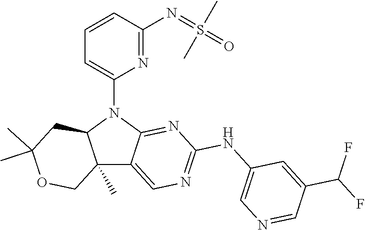 [SMILES: CC1(C)C[C@H]2N(c3cccc(N=S(C)(C)=O)n3)c3nc(Nc4cncc(C(F)F)c4)ncc3[C@@]2(C)CO1] | 815 | 1H NMR (300 MHz, DMSO-d6) δ 9.82 (s, 1H), 8.93 (s, 1H), 8.60 (s, 1H), 8.31 (s, 1H), 8.12 (s, 1H), 8.00 (d, J = 8.1 Hz, 1H), 7.55 (t, J = 7.8 Hz, 1H), 7.37-6.92 (m, 1H), 6.35 (d, J = 7.8 Hz, 1H), 4.65- 4.53 (m, 1H), 3.91-3.81 (m, 1H), 3.73- 3.60 (m, 1H), 3.49 (s, 3H), 3.30 (s, 3H), 2.49-2.40 (m, 1H), 1.63-1.45 (m, 1H), 1.30 (s, 3H), 1.22 (s, 3H), 1.02 (s, 3H). | LCMS (ES, m/z): 530.15 [M + H]+, Rt 0.640 min. |
| 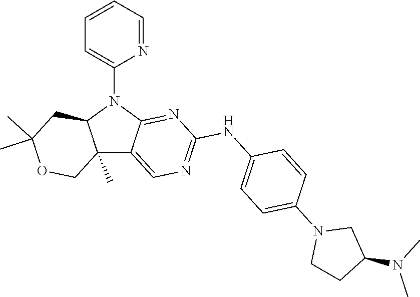 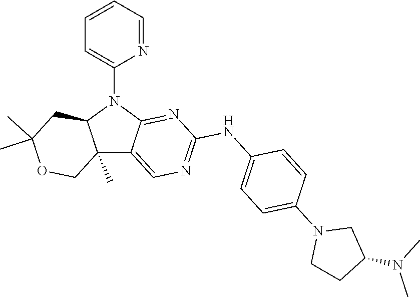 | 820, 821 | 1H NMR (300 MHz, DMSO-d6) δ 8.92 (s, 1H), 8.64 (d, J = 8.4 Hz, 1H), 8.37-8.31 (m, 1H), 7.97 (s, 1H), 7.80-7.70 (m, 1H), 7.45 (d, J = 8.7 Hz, 2H), 7.05-6.98 (m, 1H), 6.51 (d, J = 8.7 Hz, 2H), 4.72-4.61 (m, 1H), 3.82-3.61 (m, 2H), 3.45-3.35 (m, 1H), 3.30-3.14 (m, 1H), 3.01 (t, J = 8.1 Hz, 1H), 2.87-2.74 (m, 1H), 2.87- 2.74 (m, 1H), 2.26-2.07 (m, 8H), 1.89- 1.72 (m, 1H), 1.65-1.53 (m, 1H), 1.23 (s, 3H), 1.20 (s, 3H), 0.99 (s, 3H). 1H NMR (300 MHz, DMSO-d6) δ 8.93 (s, 1H), 8.65 (d, J = 8.4 Hz, 1H), 8.40-8.31 (m, 1H), 7.98 (s, 1H), 7.82-7.70 (m, 1H), 7.52-7.42 (m, 2H), 7.07-6.97 (m, 1H), 6.57-6.46 (m, 2H), 4.72-4.62 (m, 1H), 3.84-3.74 (m, 1H), 3.69-3.59 (m, 1H), 3.47-3.35 (m, 1H), 3.33-3.15 (m, 2H), 3.08-2.96 (m, 1H), 2.87-2.71 (m, 1H), 2.28-2.07 (m, 8H), 1.90-1.70 (m, 1H), 1.66-1.53 (m, 1H), 1.23 (s, 3H), 1.20 (s, 3H), 1.00 (s, 3H). | LCMS (ES, m/z): 500.20 [M + H]+, Rt 0.620 min. LCMS (ES, m/z): 500.30 [M + H]+, Rt 0.642 min. |
| 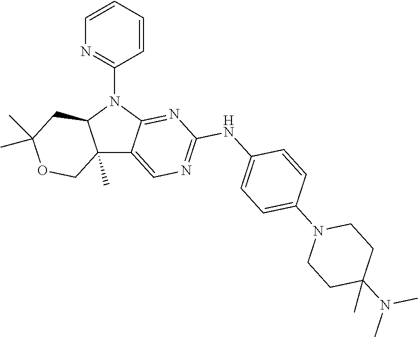 | 822 | 1H NMR (300 MHz, DMSO-d6) δ 9.03 (s, 1H), 8.63 (d, J = 8.7 Hz, 1H), 8.36 (d, J = 4.8 Hz, 1H), 8.00 (s, 1H), 7.78 (t, J = 1.5 Hz, 1H), 7.52 (d, J = 9.0 Hz, 2H), 7.04 (dd, J = 6.3 Hz, 1H), 6.88 (d, J = 9.0 Hz, 2H), 4.81-4.53 (m, 1H), 3.86-3.72 (m, 1H), 3.71-3.60 (m, 1H), 3.19-3.09 (m, 2H), 3.08-2.95 (m, 2H), 2.23-2.08 (m, 7H), 1.87-1.72 (m, 2H), 1.68-1.46 (m, 3H), 1.24 (s, 3H), 1.21 (s, 3H), 1.00 (s, 3H), 0.88 (s, 3H). | LCMS (ES, m/z): 528.35 [M + H]+, Rt 1.350 min. |
| 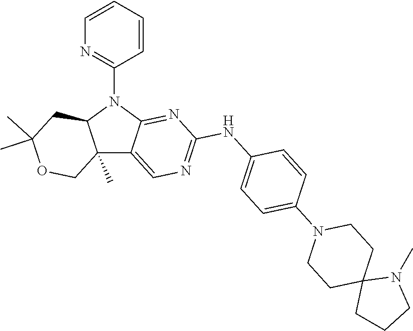 | 823 | 1H NMR (300 MHz, DMSO-d6) δ 9.10 (s, 1H), 8.63 (d, J = 8.4 Hz, 1H), 8.40-8.34 (m, 1H), 8.01 (s, 1H), 7.84-7.72 (m, 1H), 7.55 (d, J = 9.0 Hz, 2H), 7.07-7.00 (m, 1H), 6.93 (d, J = 9.0 Hz, 2H), 4.72-4.64 (m, 1H), 3.85-3.76 (m, 1H), 3.71-3.60 (m, 3H), 3.19-3.11 (m, 2H), 2.77-2.62 (m, 2H), 2.55 (s, 3H), 2.21-2.11 (m, 1H), 2.01-1.81 (m, 6H), 1.68-1.52 (m, 3H), 1.24 (s, 3H), 1.20 (s, 3H), 0.99 (s, 3H). | LCMS (ES) m/z): 540.35 [M + H]+, Rt 1.077 min. |
| 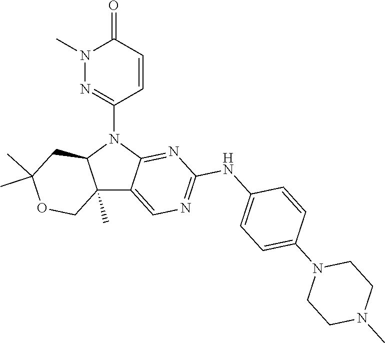 | 826 | 1H NMR (400 MHz, DMSO-d6) δ 9.11 (s, 1H), 8.72-8.65 (m, 1H), 8.00 (s, 1H), 7.47 (d, J = 9.2 Hz, 2H), 7.04 (d, J = 10.0 Hz, 1H), 6.88 (d, J = 9.2 Hz, 2H), 4.36- 4.27 (m, 1H), 3.72-3.66 (m, 1H), 3.61 (s, 3H), 3.60-3.56 (m, 1H), 3.10-3.01 (m, 4H), 2.47-2.39 (m, 4H), 2.22 (s, 3H), 2.12-2.01 (m, 1H), 1.82-1.71 (m, 1H), 1.26 (s, 3H), 1.18 (s, 3H), 1.03 (s, 3H). | LCMS (ES, m/z): 517.25 [M + H]+, Rt 0.655 min. |
| 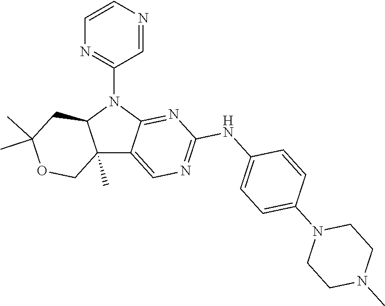 | 827 | 1H NMR (300 MHz, DMSO-d6) δ 9.95 (s, 1H), 9.24 (s, 1H), 8.42-8.37 (m, 1H), 8.25 (d, J = 2.7 Hz, 1H), 8.08 (s, 1H), 7.54 (d, J = 9.0 Hz, 2H), 6.88 (d, J = 9.0 Hz, 2H), 4.64-4.55 (m, 1H), 3.85-3.63 (m, 2H), 3.12-3.01 (m, 4H), 2.49-3.41 (m, 4H), 2.22 (s, 3H), 2.20-2.10 (m, 1H), 1.68-1.56 (m, 1H), 1.26 (s, 3H), 1.21 (s, 3H), 1.00 (s, 3H). | LCMS (ES, m/z): 487.30 [M + H]+, Rt 0.575 min. |
| 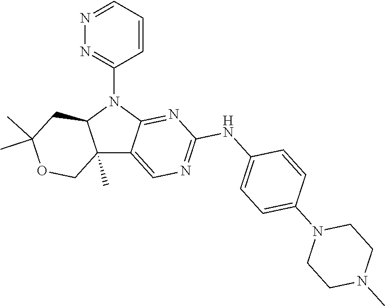 | 828 | 1H NMR (300 MHz, DMSO-d6) δ 9.16 (s, 1H), 8.96-8.85 (m, 2H), 8.09 (s, 1H), 7.71-7.62 (m, 1H), 7.50 (d, J = 9.0 Hz, 2H), 6.90 (d, J = 9.0 Hz, 2H), 4.86-4.75 (m, 1H), 3.85-3.62 (m, 2H), 3.12-3.04 (m, 4H), 2.49-2.41 (m, 4H), 2.31-2.26 (m, 1H), 2.22 (s, 3H), 1.72-1.61 (m, 1H), 1.27 (s, 3H), 1.22 (s, 3H), 1.00 (s, 3H). | LCMS (ES, m/z): 487.25 [M + H]+, Rt 0.970 min. |
| 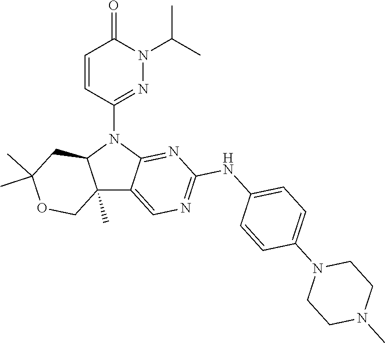 | 829 | 1H NMR (400 MHz, DMSO-d6) δ 9.11 (s, 1H), 8.72 (d, J = 10.0 Hz, 1H), 7.99 (s, 1H), 7.48 (d, J = 9.2 Hz, 2H), 7.01 (d, J = 10.0 Hz, 1H), 6.91-6.86 (m, 2H), 5.21- 5.09 (m, 1H), 4.35-4.30 (m, 1H), 3.73- 3.61 (m, 2H), 3.12-3.02 (m, 4H), 2.45 (t, J = 5.0 Hz, 4H), 2.22 (s, 3H), 2.16-2.12 (m, 1H), 1.75-1.72 (m, 1H), 1.33-1.25 (m, 9H), 1.21 (s, 3H), 1.05 (s, 3H). | LCMS (ES, m/z): 545.30 [M + H]+, Rt 0.633 min. |

### Example 702. (S)-4-methyl-3-(6-((4bR,8aR)-4b-methyl-2-((4-(4-methylpiperazin-1-yl)phenyl)amino)-4b,7,8,8a-tetrahydropyrano[3′,4:4,5]pyrrolo[2,3-d]pyrimidin-9(5H)-yl)pyridin-2-yl)oxazolidin-2-one

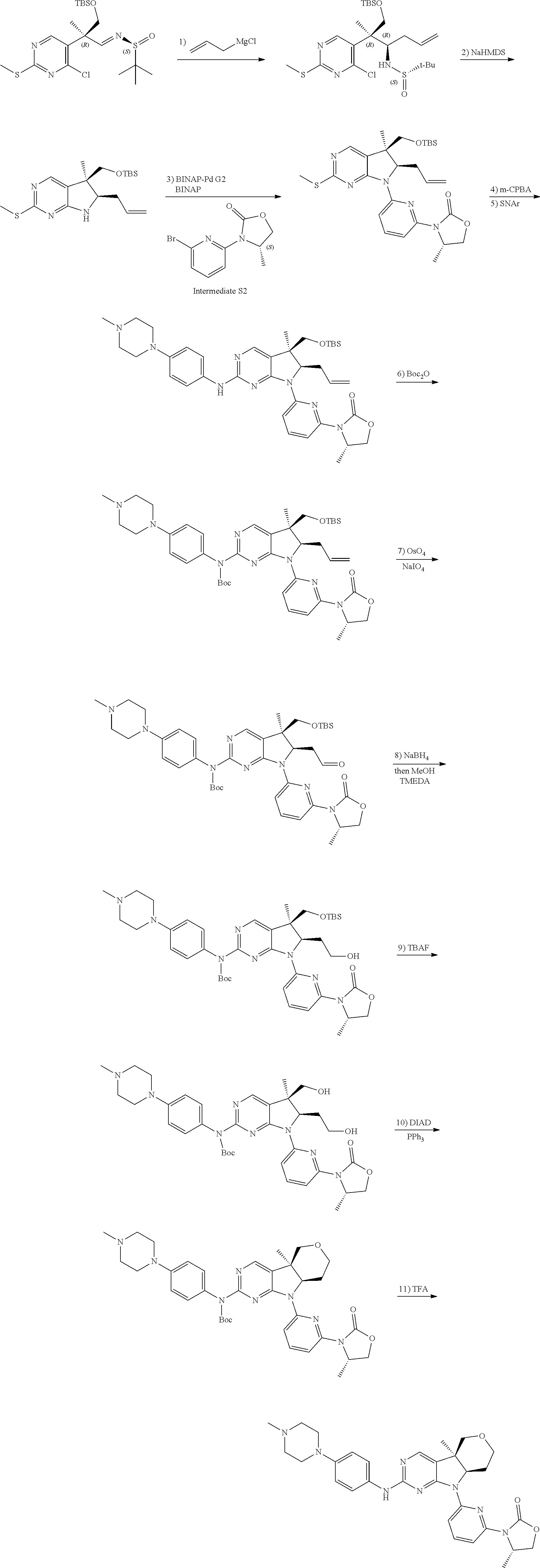

### Step 1. (S)—N-((2R,3R)-1-((tert-butyldimethylsilyl)oxy)-2-(4-chloro-2-(methylthio)pyrimidin-5-yl)-2-methylhex-5-en-3-yl)-2-methylpropane-2-sulfinamide

Following the general procedure described for Intermediate 300/step 5, (S)—N-((2R,3R)-1-((tert-butyldimethylsilyl)oxy)-2-(4-chloro-2-(methylthio)pyrimidin-5-yl)-2-methylhex-5-en-3-yl)-2-methylpropane-2-sulfinamide (340 mg, 671.62 μmol, 62% yield) was obtained as a light yellow oil, starting from (S)—N—((R,E)-3-((tert-butyldimethylsilyl)oxy)-2-(4-chloro-2-(methylthio)pyrimidin-5-yl)-2-methylpropylidene)-2-methylpropane-2-sulfinamide (Intermediate 300/step 4, 500 mg, 1.08 mmol, 1 eq.) and allylmagnesiumchloride (1 M in THF, 1.13 mL, 1.05 eq.). LCMS (ES, m/z): 506, 508 [M+H]⁺, Rt 0.990 min.

### Step 2. (5R,6R)-6-allyl-5-(((tert-butyldimethylsilyl)oxy)methyl)-5-methyl-2-(methylthio)-6,7-dihydro-5H-pyrrolo[2,3-d]pyrimidine

Following the general procedure described for Intermediate 300/step 6, (5R,6R)-6-allyl-5-(((tert-butyldimethylsilyl)oxy)methyl)-5-methyl-2-(methylthio)-6,7-dihydro-5H-pyrrolo[2,3-d]pyrimidine (230 mg, 0.63 μmol, 88% yield) was obtained as a light yellow oil, starting from (S)—N-((2R,3R)-1-((tert-butyldimethylsilyl)oxy)-2-(4-chloro-2-(methylthio)pyrimidin-5-yl)-2-methylhex-5-en-3-yl)-2-methylpropane-2-sulfinamide (360 mg, 0.71 mmol). ¹HNMR (400 MHz, DMSO-d6) δ 7.80 (s, 1H), 7.26 (s, 1H), 5.84-5.67 (m, 1H), 5.19-5.11 (m, 2H), 3.63-3.49 (m, 3H), 2.60-2.43 (m, 4H), 2.28-2.10 (m, 1H), 1.33 (s, 3H), 0.88 (s, 9H), 0.01 (d, J=7.6 Hz, 6H). LCMS (ES, m/z): 366 [M+H]⁺, Rt 0.750 min.

### Step 3. (S)-3-(6-((5R,6R)-6-allyl-5-(((tert-butyldimethylsilyl)oxy)methyl)-5-methyl-2-(methylthio)-5,6-dihydro-7H-pyrrolo[2,3-d]pyrimidin-7-yl)pyridin-2-yl)-4-methyloxazolidin-2-one

A mixture of (5R,6R)-6-allyl-5-(((tert-butyldimethylsilyl)oxy)methyl)-5-methyl-2-(methylthio)-6,7-dihydro-5H-pyrrolo[2,3-d]pyrimidine (300 mg, 820.55 μmol, 1 eq.), (4S)-3-(6-bromo-2-pyridyl)-4-methyl-oxazolidin-2-one (Intermediate 52, 232.05 mg, 902.61 μmol, 1.2 eq.), BINAP Pd G2 (76.89 mg, 82.06 μmol, 0.1 eq.), BINAP (53.40 mg, 82.06 μmol, 1 eq.) and Cs₂CO₃ (534.70 mg, 1.64 mmol, 2 eq.) in dioxane (5 mL) was stirred at 100° C. for 2 h under the N₂ atmosphere. The mixture was cooled down to room temperature. The solids were filtered and washed by EA (3×30 mL). The filtrate was concentrated under vacuum. The crude product was purified by silica gel column chromatography (eluting with 1:3 EA/PE) to afford (S)-3-(6-((5R,6R)-6-allyl-5-(((tert-butyldimethylsilyl)oxy)methyl)-5-methyl-2-(methylthio)-5,6-dihydro-7H-pyrrolo[2,3-d]pyrimidin-7-yl)pyridin-2-yl)-4-methyloxazolidin-2-one (370 mg, 682.94 μmol, 83% yield) as a yellow solid. LCMS (ES, m/z): 542 [M+H]⁺, Rt 0.875 min.

### Step 4-5. (S)-3-(6-((5R,6R)-6-allyl-5-(((tert-butyldimethylsilyl)oxy)methyl)-5-methyl-2-((4-(4-methylpiperazin-1-yl)phenyl)amino)-5,6-dihydro-7H-pyrrolo[2,3-d]pyrimidin-7-yl)pyridin-2-yl)-4-methyloxazolidin-2-one

Following the general procedures described for Examples 3 and 4/steps 4-5, (S)-3-(6-((5R,6R)-6-allyl-5-(((tert-butyldimethylsilyl)oxy)methyl)-5-methyl-2-((4-(4-methylpiperazin-1-yl)phenyl)amino)-5,6-dihydro-7H-pyrrolo[2,3-d]pyrimidin-7-yl)pyridin-2-yl)-4-methyloxazolidin-2-one (300 mg, 437.99 μmol, 66% yield) was obtained as a yellow solid, starting from (S)-3-(6-((5R,6R)-6-allyl-5-(((tert-butyldimethylsilyl)oxy)methyl)-5-methyl-2-(methylthio)-5,6-dihydro-7H-pyrrolo[2,3-d]pyrimidin-7-yl)pyridin-2-yl)-4-methyloxazolidin-2-one (360 mg, 664.48 μmol, 1 eq.). LCMS (ES, m/z): 685 [M+H]⁺, Rt 0.744 min.

### Step 6. tert-butyl ((5R,6R)-6-allyl-5-(((tert-butyldimethylsilyl)oxy)methyl)-5-methyl-7-(6-((S)-4-methyl-2-oxooxazolidin-3-yl)pyridin-2-yl)-6,7-dihydro-5H-pyrrolo[2,3-d]pyrimidin-2-yl)(4-(4-methylpiperazin-1-yl)phenyl)carbamate

To a stirred solution of (S)-3-(6-((5R,6R)-6-allyl-5-(((tert-butyldimethylsilyl)oxy)methyl)-5-methyl-2-((4-(4-methylpiperazin-1-yl)phenyl)amino)-5,6-dihydro-7H-pyrrolo[2,3-d]pyrimidin-7-yl)pyridin-2-yl)-4-methyloxazolidin-2-one (280 mg, 408.79 μmol, 1 eq.), DMAP (49.94 mg, 408.79 μmol, 1 eq.) and TEA (124.10 mg, 1.23 mmol, 3 eq.) in DCM (5 mL) was added Boc₂O (223.04 mg, 1.02 mmol, 2.5 eq.) at 0° C. The reaction mixture was stirred at room temperature for 16 h and was then concentrated under vacuum. The crude product was purified by silica gel column chromatography (eluting with 2:1 EA:PE) to afford tert-butyl ((5R,6R)-6-allyl-5-(((tert-butyldimethylsilyl)oxy)methyl)-5-methyl-7-(6-((S)-4-methyl-2-oxooxazolidin-3-yl)pyridin-2-yl)-6,7-dihydro-5H-pyrrolo[2,3-d]pyrimidin-2-yl)(4-(4-methylpiperazin-1-yl)phenyl)carbamate (260 mg, 331.19 μmol, 81% yield) as a yellow solid. LCMS (ES, m/z): 785 [M+H]⁺, Rt 0.750 min.

### Step 7. tert-butyl ((5R,6R)-5-(((tert-butyldimethylsilyl)oxy)methyl)-5-methyl-7-(6-((S)-4-methyl-2-oxooxazolidin-3-yl)pyridin-2-yl)-6-(2-oxoethyl)-6,7-dihydro-5H-pyrrolo[2,3-d]pyrimidin-2-yl)(4-(4-methylpiperazin-1-yl)phenyl)carbamate

To a stirred solution of tert-butyl ((5R,6R)-6-allyl-5-(((tert-butyldimethylsilyl)oxy)methyl)-5-methyl-7-(6-((S)-4-methyl-2-oxooxazolidin-3-yl)pyridin-2-yl)-6,7-dihydro-5H-pyrrolo[2,3-d]pyrimidin-2-yl)(4-(4-methylpiperazin-1-yl)phenyl)carbamate (250 mg, 318.45 μmol, 1 eq.) in THF (6 mL) and H₂O (2 mL) was added osmium tetroxide (8.10 mg, 31.84 μmol, 0.1 eq.) and sodium periodate (204.34 mg, 955.34 μmol, 3 eq.) in portions at 0° C. The reaction was stirred at 25° C. for 16 h. The mixture was diluted with saturated Na₂S₂O₃ (50 mL) and extracted with EA (3×50 mL). The combined organic layers were washed with brine (80 mL), dried over anhydrous Na₂SO₄, filtered and concentrated under vacuum. The crude product was purified by silica gel column chromatography (eluting with 10:1 DCM/MeOH) to afford tert-butyl ((5R,6R)-5-(((tert-butyldimethylsilyl)oxy)methyl)-5-methyl-7-(6-((S)-4-methyl-2-oxooxazolidin-3-yl)pyridin-2-yl)-6-(2-oxoethyl)-6,7-dihydro-5H-pyrrolo[2,3-d]pyrimidin-2-yl)(4-(4-methylpiperazin-1-yl)phenyl)carbamate (190 mg, 241.41 μmol, 76% yield) as a yellow solid. LCMS (ES, m/z): 787 [M+H]⁺, Rt 0.731 min.

### Step 8. tert-butyl ((5R,6R)-5-(((tert-butyldimethylsilyl)oxy)methyl)-6-(2-hydroxyethyl)-5-methyl-7-(6-((S)-4-methyl-2-oxooxazolidin-3-yl)pyridin-2-yl)-6,7-dihydro-5H-pyrrolo [2,3-d]pyrimidin-2-yl)(4-(4-methylpiperazin-1-yl)phenyl)carbamate

To a stirred solution of tert-butyl ((5R,6R)-5-(((tert-butyldimethylsilyl)oxy)methyl)-5-methyl-7-(6-((S)-4-methyl-2-oxooxazolidin-3-yl)pyridin-2-yl)-6-(2-oxoethyl)-6,7-dihydro-5H-pyrrolo[2,3-d]pyrimidin-2-yl)(4-(4-methylpiperazin-1-yl)phenyl)carbamate (100 mg, 127.06 μmol, 1 eq.) in THF (5 mL) was added NaBH₄ (9.61 mg, 254.12 μmol, 2 eq.) in portions at 0° C. The reaction mixture was stirred at 25° C. for 2 h and was then concentrated under vacuum. To the residue was added MeOH (5 mL) and TMEDA (44.29 mg, 381.18 μmol, 3 eq) and the resulting mixture was stirred at 25° C. for 2 h. The crude tert-butyl ((5R,6R)-5-(((tert-butyldimethylsilyl)oxy)methyl)-6-(2-hydroxyethyl)-5-methyl-7-(6-((S)-4-methyl-2-oxooxazolidin-3-yl)pyridin-2-yl)-6,7-dihydro-5H-pyrrolo[2,3-d]pyrimidin-2-yl)(4-(4-methylpiperazin-1-yl)phenyl)carbamate (120 mg) was obtained as a yellow solid upon concentration of the solvents under reduced pressure. LCMS (ES, m/z): 789 [M+H]⁺, Rt 0.697 min.

### Step 9. tert-butyl ((5R,6R)-6-(2-hydroxyethyl)-5-(hydroxymethyl)-5-methyl-7-(6-((S)-4-methyl-2-oxooxazolidin-3-yl)pyridin-2-yl)-6,7-dihydro-5H-pyrrolo[2,3-d]pyrimidin-2-yl)(4-(4-methylpiperazin-1-yl)phenyl)carbamate

A solution of tert-butyl ((5R,6R)-5-(((tert-butyldimethylsilyl)oxy)methyl)-6-(2-hydroxyethyl)-5-methyl-7-(6-((S)-4-methyl-2-oxooxazolidin-3-yl)pyridin-2-yl)-6,7-dihydro-5H-pyrrolo[2,3-d]pyrimidin-2-yl)(4-(4-methylpiperazin-1-yl)phenyl)carbamate (100 mg, 126.73 μmol, 1 eq.) in TBAF (1M in THF, 5 mL) was stirred at 25° C. for 2 h and was then concentrated under vacuum. The crude product was purified by reverse phase chromatography with the following conditions: (Column: C18, 20 g, 40-60 nm; Mobile Phase A: water (10 mM NH₄HCO₃), Mobile Phase B: MeCN (45% to 65% in 7 min); 254 nm) to afford tert-butyl ((5R,6R)-6-(2-hydroxyethyl)-5-(hydroxymethyl)-5-methyl-7-(6-((S)-4-methyl-2-oxooxazolidin-3-yl)pyridin-2-yl)-6,7-dihydro-5H-pyrrolo[2,3-d]pyrimidin-2-yl)(4-(4-methylpiperazin-1-yl)phenyl)carbamate (40 mg, 47% yield) as a yellow solid. LCMS (ES, m/z): 675 [M+H]⁺, Rt 0.544 min.

### Step 10. tert-butyl ((4bR,8aR)-4b-methyl-9-(6-((S)-4-methyl-2-oxooxazolidin-3-yl)pyridin-2-yl)-4b,5,7,8,8a,9-hexahydropyrano[3′,4′:4,5]pyrrolo[2,3-d]pyrimidin-2-yl)(4-(4-methylpiperazin-1-yl)phenyl)carbamate

To a solution of tert-butyl ((5R,6R)-6-(2-hydroxyethyl)-5-(hydroxymethyl)-5-methyl-7-(6-((S)-4-methyl-2-oxooxazolidin-3-yl)pyridin-2-yl)-6,7-dihydro-5H-pyrrolo[2,3-d]pyrimidin-2-yl)(4-(4-methylpiperazin-1-yl)phenyl)carbamate (35 mg, 51.87 μmol, 1 eq.) and PPh₃ (27.21 mg, 103.74 μmol, 2 eq.) in THF (2 mL) was added dropwise DIAD (20.98 mg, 103.74 μmol, 2 eq.) at 0° C. under nitrogen atmosphere. The reaction was stirred at 50° C. for 2 h. The resulting mixture was cooled down to room temperature and concentrated under vacuum. The crude product was purified by Prep-TLC (eluting with 1:1 EA:PE) to afford tert-butyl ((4bR,8aR)-4b-methyl-9-(6-((S)-4-methyl-2-oxooxazolidin-3-yl)pyridin-2-yl)-4b,5,7,8,8a,9-hexahydropyrano[3′,4′:4,5]pyrrolo[2,3-d]pyrimidin-2-yl)(4-(4-methylpiperazin-1-yl)phenyl)carbamate (25 mg, 38.06 μmol, 73% yield) as a yellow solid. LCMS (ES, m/z): 657 [M+H]⁺, Rt 0.638 min.

### Step 11. (S)-4-methyl-3-(6-((4bR,8aR)-4b-methyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)pyridin-2-yl)oxazolidin-2-one

A solution of tert-butyl ((4bR,8aR)-4b-methyl-9-(6-((S)-4-methyl-2-oxooxazolidin-3-yl)pyridin-2-yl)-4b,5,7,8,8a,9-hexahydropyrano[3′,4′:4,5]pyrrolo[2,3-d]pyrimidin-2-yl)(4-(4-methylpiperazin-1-yl)phenyl)carbamate (23 mg, 35.02 μmol, 1 eq.) in mixture of TFA (0.5 mL) and DCM (1.5 mL) was stirred for 2 h at 25° C. The resulting mixture was concentrated under reduced pressure and the residue was purified by Prep-HPLC: (Column: XBridge Shield RP18 OBD Column, 30*150 mm/5 μm; Mobile Phase A: Water(10 mmol/L NH₄HCO₃), Mobile Phase B: ACN; Flow rate: 60 mL/min; Gradient: 43% to 63% B in 7 min; 254 nm) to afford (S)-4-methyl-3-(6-((4bR,8aR)-4b-methyl-2-((4-(4-methylpiperazin-1-yl)phenyl)amino)-4b,7,8,8a-tetrahydropyrano[3′,4′:4,5]pyrrolo[2,3-d]pyrimidin-9(5H)-yl)pyridin-2-yl)oxazolidin-2-one (7.1 mg, 36% yield) as a white solid.

¹H NMR (400 MHz, DMSO-d6) δ 9.14 (s, 1H), 8.29 (d, J=8.0 Hz, 5H), 8.08 (s, 1H), 7.84 (t, J=8.0 Hz, 1H), 7.63 (d, J=8.0 Hz, 1H), 7.55 (d, J=8.8 Hz, 2H), 6.91-6.88 (m, 2H), 5.01-4.88 (m, 1H), 4.69-4.60 (m, 1H), 4.53 (t, J=8.4 Hz, 1H), 4.18-4.07 (m, 2H), 3.75-3.68 (m, 1H), 3.55-3.51 (m, 2H), 3.08-3.02 (m, 4H), 2.50-2.47 (m, 4H), 2.29-2.21 (m, 4H), 1.59-1.42 (m, 1H), 1.36 (d, J=6.4 Hz, 3H), 1.12 (s, 3H). LCMS (ES, m/z): 557 [M+H]⁺, Rt 0.590 mi.

TABLE 25
Using appropriate intermediates and reaction conditions, the following
examples were prepared in the similar manner to Example 702.

| Structure | EX # | ¹H NMR | LCMS |
|---|---|---|---|
| 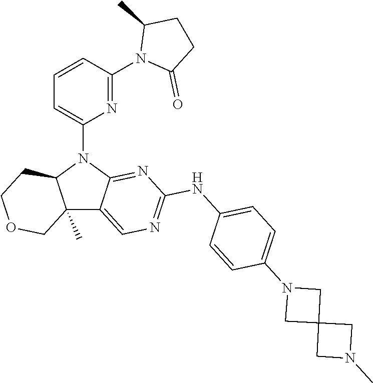 [SMILES: C[C@H]1CCC(=O)N1c1cccc(N2c3nc(Nc4ccc(N5CC6(CN(C)C6)C5)cc4)ncc3[C@@]3(C)COCC[C@@H]23)n1] | 776 | 1H NMR (400 MHz, DMSO-d6) δ 9.01 (s, 1H), 8.26 (d, J = 7.6 Hz, 1H), 8.04 (s, 1H), 7.83-7.75 (m, 2H), 7.47 (d, J = 8.4 Hz, 2H), 6.40-6.38 (m, 2H), 4.85-4.81 (m, 1H), 4.62-4.58 (m, 1H), 4.12- 4.09 (m, 1H), 3.83-3.78 (m, 4H), 3.72-3.68 (m, 1H), 3.70-3.44 (m, 2H), 3.29-3.24 (m, 4H), 2.80- 2.74 (m, 1H), 2.47-2.39 (m, 1H), 2.30-2.22 (m, 2H), 2.20 (s, 3H), 1.77-1.67 (m, 1H), 1.51-1.49 (m, 1H), 1.26 (d, J = 6.0 Hz, 3H) 1.11 (s, 3H) | LCMS (ES, m/z): 567.25 [M + H]+, Rt 0.898 min. |
| 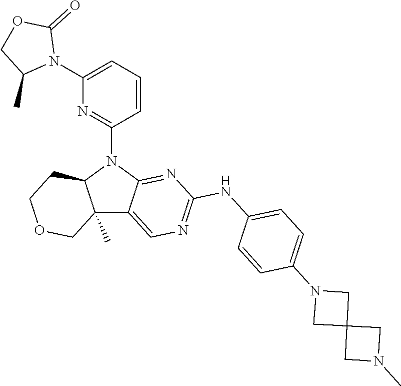 [SMILES: C[C@H]1COC(=O)N1c1cccc(N2c3nc(Nc4ccc(N5CC6(CN(C)C6)C5)cc4)ncc3[C@@]3(C)COCC[C@@H]23)n1] | 784 | 1H NMR (400 MHz, DMSO-d6) δ 9.01 (s, 1H), 8.27 (d, J = 8.0 Hz, 1H), 8.05 (s, 1H), 7.80 (t, J = 8.4 Hz, 1H), 7.62 (d, J = 8.4 Hz, 1H), 7.47 (d, J = 8.4 Hz, 2H), 6.39 (d, J = 8.4 Hz, 2H), 4.95-4.89 (m, 1H), 4.61-4.59 (m, 1H), 4.55-4.51 (m, 1H), 4.21-3.99 (m, 2H), 3.84-3.76 (m, 4H), 3.74- 3.66 (m, 1H), 3.57-3.42 (m, 2H), 3.28-2.21 (m, 4H), 2.29-2.21 (m, 1H), 2.19 (s, 3H), 1.54-1.47 (s, 1H), 1.36 (d, J = 6.0 Hz, 3H), 1.12 (s, 3H). | LCMS (ES, m/z): 569.25 [M + H]+, Rt 0.608 min. |
| 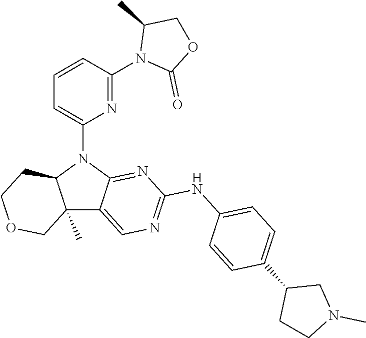 [SMILES: C[C@H]1COC(=O)N1c1cccc(N2c3nc(Nc4ccc([C@H]5CCN(C)C5)cc4)ncc3[C@@]3(C)COCC[C@@H]23)n1] | 793, 794 | 1H NMR (400 MHz, DMSO-d6) δ 9.32 (s, 1H), 8.30 (d, J = 8.0 Hz, 1H), 8.12 (s, 1H), 7.84 (t, J = 8.0 Hz, 1H), 7.65-7.63 (m, 3H), 7.19-7.17 (m, 2H), 4.96-4.91 (m, 1H), 4.66-4.62 (m, 1H), 4.53 (t, J = 8.0 Hz, 1H), 4.15-4.10 (m, 2H), 3.74-3.69 (m, 1H), 3.62-3.43 (m, 2H), 3.30-3.22 (m, 1H), 2.85 (t, J = 8.4 Hz, 1H), 2.65-2.54 (m, 2H), 2.38- 2.34 (m, 1H), 2.29-2.19 (m, 5H), 1.79-1.70 (m, 1H), 1.56-1.46 (m, 1H), 1.36 (d, J = 6.4 Hz, 3H), 1.13 (s, 3H). | LCMS (ES, m/z): 542.25 [M + H]+, Rt 0.600 min. |
| 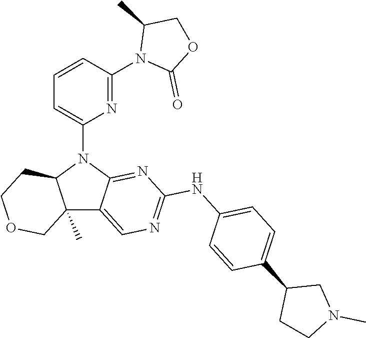 [SMILES: C[C@H]1COC(=O)N1c1cccc(N2c3nc(Nc4ccc([C@@H]5CCN(C)C5)cc4)ncc3[C@@]3(C)COCC[C@@H]23)n1] | | 1H NMR (400 MHz, DMSO-d6) δ 9.32 (s, 1H), 8.30 (d, J = 8.4 Hz, 1H), 8.13-8.12 (m, 1H), 7.86- 7.82 (m, 1H), 7.65-7.63 (m, 3H), 7.19-7.17 (m, 2H), 4.96-4.92 (m, 1H), 4.66-4.51 (m, 2H), 4.15- 4.11 (m, 2H), 3.73-3.70 (m, 1H), 3.56-3.44 (m, 2H), 3.28-3.24 (m, 1H), 2.85 (t, J = 8.0 Hz, 1H), 2.65-2.57 (m, 2H), 2.36 (t, J = 8.2 Hz, 1H), 2.29- 2.25 (m, 5H), 1.77-1.72 (m, 1H), 1.52-1.50 (m, 1H), 1.38-1.36 (m, 3H), 1.13 (s, 3H). | LCMS (ES, m/z): 542.25 [M + H]+, Rt 0.592 min. |

### Examples 396 and 397: (1R,2R)—N-((4bR,8aR)-9-(6-((dimethyl(oxo)-l6-sulfaneylidene)amino)pyridin-2-yl)-4b,7,7-trimethyl-4b,5,7,8,8a,9-hexahydropyrano[3′,4′:4,5]pyrrolo[2,3-d]pyrimidin-2-yl)-2-phenylcyclopropane-1-carboxamide and (1S,2S)—N-((4bR,8aR)-9-(6-((dimethyl(oxo)-l6-sulfaneylidene)amino)pyridin-2-yl)-4b,7,7-trimethyl-4b,5,7,8,8a,9-hexahydropyrano[3′,4′:4,5]pyrrolo[2,3-d]pyrimidin-2-yl)-2-phenylcyclopropane-1-carboxamide

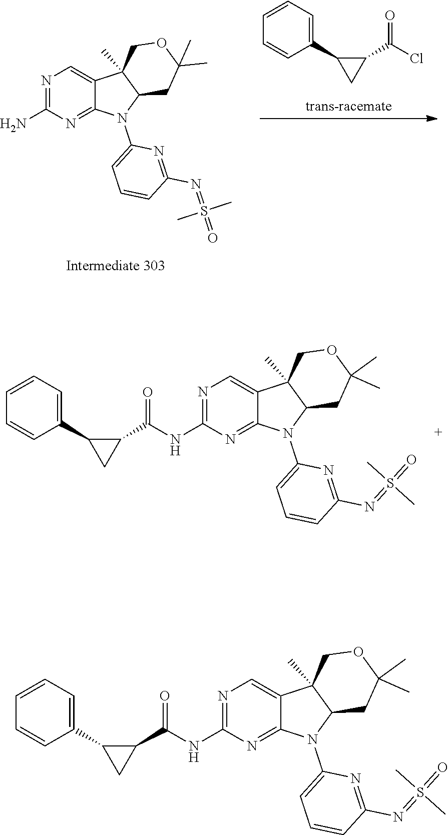 [SMILES: CC1(C)C[C@H]2N(c3cccc(N=S(C)(C)=O)n3)c3nc(N)ncc3[C@@]2(C)CO1 ; O=C(Cl)[C@@H]1C[C@H]1c1ccccc1 ; CC1(C)C[C@H]2N(c3cccc(N=S(C)(C)=O)n3)c3nc(NC(=O)[C@@H]4C[C@H]4c4ccccc4)ncc3[C@@]2(C)CO1 ; CC1(C)C[C@H]2N(c3cccc(N=S(C)(C)=O)n3)c3nc(NC(=O)[C@H]4C[C@@H]4c4ccccc4)ncc3[C@@]2(C)CO1]

To a stirred mixture of ((6-((4bR,8aR)-2-amino-4b,7,7-trimethyl-4b,7,8,8a-tetrahydropyrano[3′,4′:4,5]pyrrolo[2,3-d]pyrimidin-9(5H)-yl)pyridin-2-yl)imino)dimethyl-l6-sulfanone (Intermediate 303, 50 mg, 124.22 μmol, 1 eq.) in pyridine (3 mL) was added trans-2-phenylcyclopropanecarbonyl chloride (67.3 mg, 373 μmol, 3 eq.) in DCM at 0° C. The resulting mixture was stirred for 24 h at 80° C. The reaction was monitored by LC-MS. The resulting mixture was allowed to cool down to room temperature. The resulting mixture was concentrated under reduced pressure. The residue was purified by Prep-HPLC(Column: XBridge Prep OBD C18 Column, 30×150 mm, 5 μm; Mobile Phase A: Water (10 mmol/L NH₄HCO₃), Mobile Phase B: MeCN (25% to 55% in 7 min); Flow rate: 60 mL/min; Wave Length: 254 nm) to afford the racemic product. The racemic mixture was separated by Prep-CHIRAL-HPLC(Column: CHIRALPAK IG, 2×25 cm, 5 μm; Mobile Phase A: Hex (0.5% 2M NH₃-MeOH), Mobile Phase B: EtOH (40% to 40% in 28 min); Flow rate: 18 mL/min; Wave Length: 220/254 nm; RT1(min): 18.333; RT2(min): 27.291) to afford (1R,2R)—N-((4bR,8aR)-9-(6-((dimethyl(oxo)-l6-sulfaneylidene)amino)pyridin-2-yl)-4b,7,7-trimethyl-4b,5,7,8,8a,9-hexahydropyrano[3′,4′:4,5]pyrrolo[2,3-d]pyrimidin-2-yl)-2-phenylcyclopropane-1-carboxamide (10.7 mg, 19.56 μmol, 32% yield) as a light-yellow solid and (1S,2S)—N-((4bR,8aR)-9-(6-((dimethyl(oxo)-l6-sulfaneylidene)amino)pyridin-2-yl)-4b,7,7-trimethyl-4b,5,7,8,8a,9-hexahydropyrano[3′,4′:4,5]pyrrolo[2,3-d]pyrimidin-2-yl)-2-phenylcyclopropane-1-carboxamide(10.8 mg, 19.69 μmol, 32% yield) as a light-yellow solid.

Example 396: ¹H NMR (400 MHz, DMSO-d6) δ 10.66 (s, 1H), 8.32 (d, J=8.0 Hz, 1H), 8.14 (s, 1H), 7.52 (t, J=8.0 Hz, 1H), 7.31-7.27 (m, 2H), 7.21-7.16 (m, 3H), 6.32 (d, J=8.0 Hz, 1H), 4.59-4.56 (m, 1H), 3.87-3.84 (m, 1H), 3.68-3.65 (m, 1H), 3.49 (s, 3H), 3.34 (s, 3H), 2.49-2.39 (m, 3H), 1.55-1.48 (m, 2H), 1.38-1.33 (m, 1H), 1.28 (s, 3H), 1.22 (s, 3H), 1.00 (s, 3H). LCMS (ES, m/z): 547 [M+H]⁺, Rt1.488 min.

Example 397: ¹H NMR (400 MHz, DMSO-d6) δ 10.67 (s, 1H), 8.33 (d, J=8.4 Hz, 1H), 8.14 (s, 1H), 7.52 (t, J=8.0 Hz, 1H), 7.31-7.27 (m, 2H), 7.21-7.17 (m, 3H), 6.32 (d, J=7.6 Hz, 1H), 4.59-4.56 (m, 1H), 3.88-3.85 (m, 1H), 3.68-3.65 (m, 1H), 3.49 (s, 3H), 3.34 (s, 3H), 2.48-2.39 (m, 3H), 1.54-1.48 (m, 2H), 1.38-1.33 (m, 1H), 1.27 (s, 3H), 1.23 (s, 3H), 1.00 (s, 3H). LCMS (ES, m/z): 547 [M+H]⁺, Rt0.766 min.

Absolute stereochemistry of Examples 396 and 397 was arbitrarily assigned upon the separation of racemic mixture by chiral-HPLC.

Using appropriate intermediates and reaction conditions, the following examples in Table 26 were prepared in the similar manner to Examples 396 and 397.

TABLE 26

| Structure | EX # | ¹H NMR | LCMS |
|---|---|---|---|
| 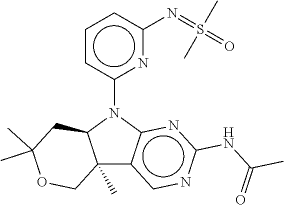 | 398 | 1H NMR (300 MHz, DMSO-d6) δ 10.30 (s, 1H), 8.27 (d, J = 8.1 Hz, 1H), 8.13 (s, 1H), 7.53 (t, J = 8.1 Hz, 1H), 6.32 (d, J = 7.5 Hz, 1H), 4.60-4.55 (m, 1H), 3.87- 3.82 (m, 1H), 3.66-3.61 (m, 1H), 3.49 (s, 3H), 3.29 (s, 3H), 2.50-2.44 (m, 1H), 2.17 (s, 3H), 1.56-1.48 (m, 1H), 1.28 (s, 3H), 1.23 (s, 3H), 1.00 (s, 3H). | LCMS (ES, m/z): 445 [M + H]+, Rt 1.099 min. |
| 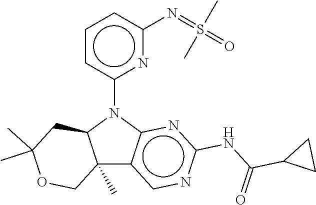 | 399 | 1H NMR (300 MHz, DMSO-d6) δ 10.57 (s, 1H), 8.30 (d, J = 8.1 Hz, 1H), 8.13 (s, 1H), 7.52 (t, J = 8.1 Hz, 1H), 6.31 (d, J = 7.2 Hz, 1H), 4.57-4.56 (m, 1H), 3.88-3.84 (m, 1H), 3.68-3.64 (m, 1H), 3.48 (s, 3H), 3.28 (s, 3H), 2.49-2.44 (m, 1H), 2.19-2.06 (m, 1H), 1.62-1.52 (m, 1H), 1.27(s, 3H), 1.22 (s, 3H), 1.00 (s, 3H), 0.83-0.78 (m, 4H). | LCMS (ES, m/z): 471 [M + H]+, Rt 1.171 min. |
| 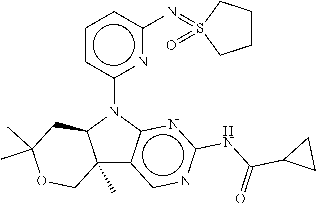 | 400 | 1H NMR (300 MHz, DMSO-d6) δ 10.61 (s, 1H), 8.34 (d, J = 7.8 Hz, 1H), 8.15 (s, 1H), 7.52 (t, J = 7.8 Hz, 1H), 6.38-6.36 (m, 1H), 4.65-4.60 (m, 1H), 3.88-3.83 (m, 1H), 3.70-3.64 (m, 2H), 3.49-3.47 (m, 1H), 3.35-3.25 (m, 2H), 2.44-2.37 (m, 1H), 2.27-2.08 (m, 5H), 1.55-1.48 (m, 1H), 1.26 (s, 3H), 1.23 (s, 3H), 1.21 (s, 3H), 0.98-0.78 (m, 4H). | LCMS (ES, m/z): 497 [M + H]+, Rt 0.652 min. |
| 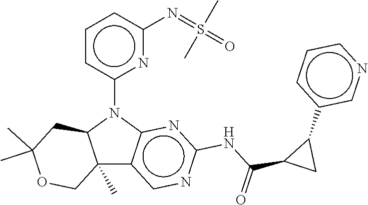 | 401 | 1H NMR (400 MHz, DMSO-d6) δ 10.69 (s, 1H), 8.49 (d, J = 2.0 Hz, 1H), 8.41 (d, J = 2.0 Hz, 1H), 8.33 (d, J = 8.0 Hz, 1H), 8.14 (s, 1H), 7.56-7.50 (m, 2H), 7.34- 7.31 (m, 1H), 6.32 (d, J = 7.6 Hz, 1H), 4.59-4.56 (m, 1H), 3.88-3.85 (m, 1H), 3.68-3.65 (m, 1H), 3.49 (s, 3H), 3.29 (s, 3H), 2.51-2.43 (m, 3H), 1.56-1.41 (m, 3H), 1.27 (s, 3H), 1.22 (s, 3H), 1.00 (s, 3H). | LCMS (ES, m/z): 548 [M + H]+, Rt 0.993 min |
| 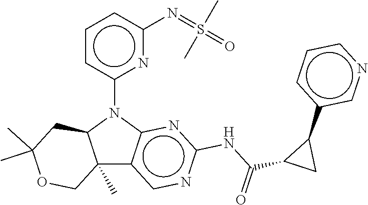 | 402 | 1H NMR (400 MHz, DMSO-d6) δ 10.69 (s, 1H), 8.49 (d, J = 2.0 Hz, 1H), 8.41 (d, J = 2.0 Hz, 1H), 8.32 (d, J = 8.4 Hz, 1H), 8.14 (s, 1H), 7.55-7.49 (m, 2H), 7.33- 7.30 (m, 1H), 6.32 (d, J = 7.6 Hz, 1H), 4.59-4.56 (m, 1H), 3.87-3.84 (m, 1H), 3.68-3.65 (m, 1H), 3.49 (s, 3H), 3.29 (s, 3H), 2.51-2.43 (m, 3H), 1.57-1.42 (m, 3H), 1.28 (s, 3H), 1.22 (s, 3H), 1.00 (s, 3H). | LCMS (ES, m/z): 548 [M + H]+, Rt 1.064 min |
| 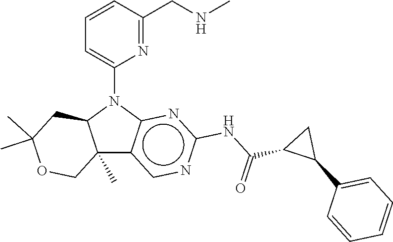 | 760, 761 | 1H NMR (400 MHz, DMSO-d6) δ 10.70 (s, 1H), 8.72 (d, J = 8.4 Hz, 1H), 8.15 (s, 1H), 7.72 (t, J = 8 Hz, 1H), 7.34-7.26 (m, 2H), 7.24-7.15 (m, 3H), 7.09- 7.01 (m, 1H), 4.75-4.72 (m, 1H), 3.86-3.78 (m, 1H), 3.72-3.64 (m, 3H), 2.49-2.37 (m, 2H), 2.33 (s, 3H), 2.23-2.19 (m, 1H), 1.68-1.61 (m, 1H), 1.56-1.47 (m, 1H), 1.40-1.31 (m, 1H), 1.26 (s, 3H), 1.21 (s, 3H), 0.99 (s, 3H). | LCMS (ES, m/z): 499.20 [M + H]+, Rt 0.650 min. |
| 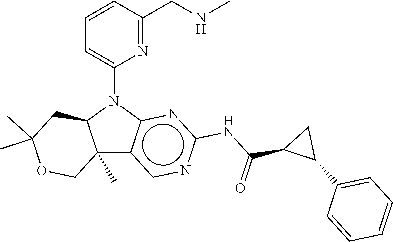 | | 1H NMR (400 MHz, DMSO-d6) δ 10.70 (s, 1H), 8.72 (d, J = 8.4 Hz, 1H), 8.15 (s, 1H), 7.72 (t, J = 8 Hz, 1H), 7.36-7.27 (m, 2H), 7.24-7.15 (m, 3H), 7.09- 7.01 (m, 1H), 4.75-4.72 (m, 1H), 3.86-3.78 (m, 1H), 3.72-3.64 (m, 3H), 2.49-2.37 (m, 2H), 2.33 (s, 3H), 2.23-2.19 (m, 1H), 1.68-1.61 (m, 1H), 1.56-1.47 (m, 1H), 1.40-1.31 (m, 1H), 1.28 (s, 3H), 1.21 (s, 3H), 0.99 (s, 3H). | LCMS (ES, m/z): 499.25 [M + H]+, Rt 1.238 min. |
| 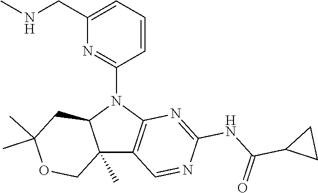 [SMILES: CNCc1cccc(N2c3nc(NC(=O)C4CC4)ncc3[C@@]3(C)COC(C)(C)C[C@@H]23)n1] | 762 | 1H NMR (400 MHz, DMSO-d6) δ 10.65 (s, 1H), 8.72 (d, J = 8.4 Hz, 1H), 8.16 (s, 1H), 7.73 (t, J = 8.0 Hz, 1H), 7.04 (d, J = 7.6 Hz, 1H), 4.75-4.72 (m, 1H), 3.82-3.79 (m, 1H), 3.75-3.64 (m, 3H), 2.34 (s, 3H), 2.23-2.18 (m, 1H), 2.16-2.09 (m, 1H), 1.68-1.62 (m, 1H), 1.28 (s, 3H), 1.22 (s, 3H), 0.99 (s, 3H), 0.89-0.75 (m, 4H). | LCMS (ES, m/z): 423.15 [M + H]+, Rt 0.960 min. |
| 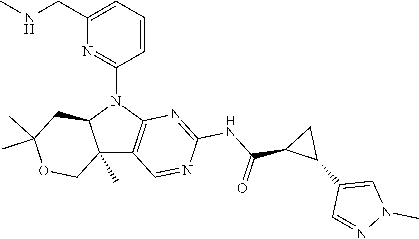 | 763, 764 | 1H NMR (400 MHz, DMSO-d6) δ 10.61 (s, 1H), 8.71 (d, J = 8.4 Hz, 1H), 8.15 (s, 1H), 7.70 (t, J = 8.0 Hz, 1H), 7.53 (s, 1H), 7.26 (s, 1H), 7.04 (d, J = 7.6 Hz, 1H), 4.74-4.71 (m, 1H), 3.81-3.75 (m, 4H), 3.69-3.65 (m, 3H), 2.33-2.28 (m, 4H), 2.22-2.18 (m, 2H), 1.68-1.62 (m, 1H), 1.40-1.35 (m, 1H), 1.28 (s, 3H), 1.23 (s, 3H), 1.18-1.14 (m, 1H), 0.99 (s, 3H). | LCMS (ES, m/z): 503.20 [M + H]+, Rt 0.988 min. |
| 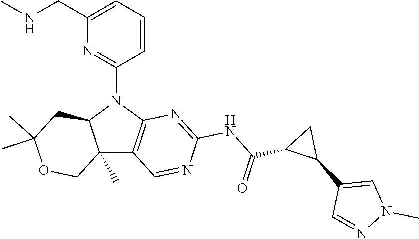 | | 1H NMR (400 MHz, DMSO-d6) δ 10.64 (s, 1H), 8.71 (d, J = 8.4 Hz, 1H), 8.15 (s, 1H), 7.70 (t, J = 8.0 Hz, 1H), 7.54 (s, 1H), 7.27 (s, 1H), 7.04 (d, J = 7.6 Hz, 1H), 4.74-4.71 (m, 1H), 3.81-3.73 (m, 4H), 3.70-3.65 (m, 3H), 2.33-2.27 (m, 3H), 2.22-2.18 (m, 3H), 1.67-1.62 (m, 1H), 1.39-1.35 (m, 1H), 1.28 (s, 3H), 1.26 (s, 3H), 1.18-1.13 (m, 1H), 0.98 (s, 3H). | LCMS (ES, m/z): 503.20 [M + H]+, Rt 0.958 min. |
| 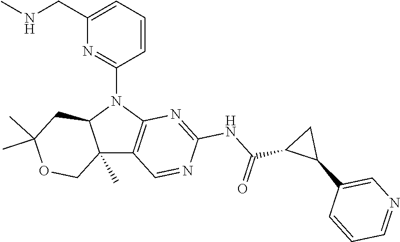 [SMILES: CNCc1cccc(N2c3nc(NC(=O)[C@@H]4C[C@H]4c4cccnc4)ncc3[C@@]3(C)COC(C)(C)C[C@@H]23)n1] | 768, 769 | 1H NMR (400 MHz, DMSO-d6) δ 10.74 (s, 1H), 8.73 (d, J = 8.4 Hz, 1H), 8.50 (s, 1H), 8.41 (d, J = 4.8 Hz, 1H),8 .15 (s, 1H), 7.72 (t, J = 8.0 Hz, 1H), 7.56-7.53 (m, 1H), 7.34-7.31 (m, 1H), 7.04 (d, J = 7.6 Hz, 1H), 4.75-4.72 (m, 1H), 3.86-3.79 (m, 1H), 3.70-3.66 (m, 3H), 2.47-2.44 (m, 2H), 2.33 (s, 3H), 2.23-2.19 (m, 1H), 1.68-1.62 (m, 1H), 1.57-1.53 (m, 1H), 1.47-1.42 (m, 1H), 1.28 (s, 3H), 1.22 (s, 3H), 0.99 (s, 3H). | LCMS (ES, m/z): 500.15 [M + H]+, Rt 0.975 min. |
| 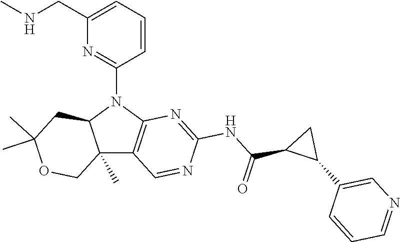 [SMILES: CNCc1cccc(N2c3nc(NC(=O)[C@H]4C[C@@H]4c4cccnc4)ncc3[C@@]3(C)COC(C)(C)C[C@@H]23)n1] | | 1H NMR (400 MHz, DMSO-d6) δ 10.74 (s, 1H), 8.72 (d, J = 8.4 Hz, 1H), 8.50 (s, 1H), 8.41 (d, J = 4.8 Hz, 1H) ,8.15 (s, 1H), 7.72 (t, J = 8.0 Hz, 1H), 7.55-7.53 (m, 1H), 7.33-7.30 (m, 1H), 7.04 (d, J = 7.6 Hz, 1H), 4.75-4.72 (m, 1H), 3.81-3.78 (m, 1H), 3.70-3.66 (m, 3H), 2.47-2.44 (m, 2H), 2.33 (s, 3H), 2.22-2.18 (m, 1H), 1.69-1.63 (m, 1H), 1.58-1.53 (m, 1H), 1.47- 1.42 (m, 1H), 1.28 (s, 3H), 1.21 (s, 3H), 0.99 (s, 3H). | LCMS (ES, m/z): 500.15 [M + H]+, Rt 0.983 min. |

### Example 403: (1R,2R)—N-((4bR,8aR)-9-(6-((dimethyl(oxo)-l6-sulfaneylidene)amino)pyridin-2-yl)-4b,7,7-trimethyl-4b,5,7,8,8a,9-hexahydropyrano[3′,4′:4,5]pyrrolo[2,3-d]pyrimidin-2-yl)-2-(1-methyl-1H-pyrazol-4-yl)cyclopropane-1-carboxamide

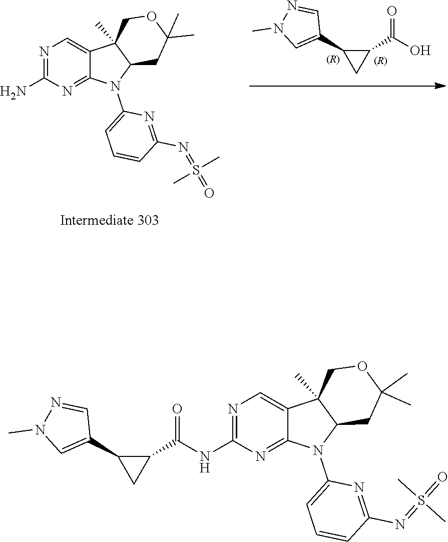

To a stirred solution of (1R,2R)-2-(1-methylpyrazol-4-yl)cyclopropanecarboxylic acid (60.00 mg, 352.94 μmol, 2.3 eq.) and (1R,9R)-8-[6-[[dimethyl (oxo)-sulfanylidene]amino]-2-pyridyl]-1,11,11-trimethyl-12-oxa-4,6,8-triazatricyclo[7.4.0.02,7]trideca-2,4,6-trien-5-amine (Intermediate 303, 60 mg, 149 μmol, 1 eq.) in DMF (1 mL) were added N,N-dimethylpyridin-4-amine (54.3 mg, 447 μmol, 3 eq.), 3-(ethyliminomethyleneamino)-N,N-dimethyl-propan-1-amine; hydrochloride (57.2 mg, 298 μmol, 2 eq.) slowly. The reaction mixture was stirred for 16 h at room temperature. The reaction was monitored by LC-MS. The mixture was diluted with water (10 mL). The resulting mixture was extracted by EA (3×10 mL). The combined organic layers were washed with brine (20 mL), dried over sodium sulfate. After filtration, the filtrated was concentrated under reduced pressure. The crude product was purified by Prep-HPLC with (Column: XBridge Prep OBD C18 Column, 30×150 mm, 5 μm; Mobile Phase A: Water (10 mmol/L NH₄HCO₃+0.1% NH₃·H₂O), Mobile Phase B: ACN; Flow rate: 60 mL/min; Gradient: 18% B to 38% B in 7 min, 38% B; Wave Length: 220 nm) to afford (1R,2R)—N-[(1S,9S)-8-[6-[[dimethyl(oxo)-sulfanylidene]amino]-2-pyridyl]-1,11,11-trimethyl-12-oxa-4,6-diazatricyclo[7.4.0.02,7]trideca-2,4,6-trien-5-yl]-2-(1-methylpyrazol-4-yl) cyclopropanecarboxamide (23.6 mg, 42.7 μmol, 29% yield) as a white solid. ¹H NMR (400 MHz, DMSO-d6) δ 10.16 (s, 1H), 8.32 (d, J=8.0 Hz, 1H), 8.14 (s, 1H), 7.54-7.48 (m, 2H), 7.27 (s, 1H), 6.32 (d, J=8.0 Hz, 1H), 4.59 (m, 1H), 3.88-3.83 (m, 1H), 3.76 (s, 3H), 3.68-3.64 (m, 1H), 3.49 (s, 3H), 3.29 (s, 3H), 2.33-2.24 (m, 2H), 2.21-2.17 (m, 1H), 1.54-1.48 (m, 1H), 1.39-1.34 (m, 1H), 1.27 (s, 3H), 1.25 (s, 3H), 1.28-1.22 (m, 1H), 0.99 (s, 3H). LCMS (ES, m/z): 551 [M+H]+, Rt 1.227 min.

Using appropriate intermediates and reaction conditions, the following examples in Table 27 were prepared in the similar manner to Example 403. Absolute stereochemistry of the examples in the following table was arbitrarily assigned.

TABLE 27

| Structure | EX # | ¹H NMR | LCMS |
|---|---|---|---|
| 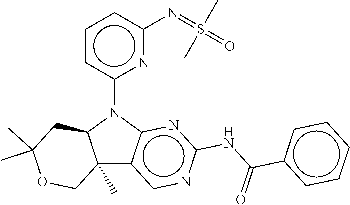 | 404 | 1H NMR (300 MHz, DMSO-d6) δ 10.73 (s, 1H), 8.23-8.21 (m, 2H), 7.95-7.92 (m, 2H), 7.59-7.48 (m, 4H), 6.31 (d, J = 7.5 Hz, 1H), 4.62-4.57 (m, 1H), 3.92-3.88 (m, 1H), 3.71-3.67 (m, 1H), 3.49 (s, 3H), 3.29 (s, 3H), 2.49-2.45 (m, 1H), 1.58- 1.50 (m, 1H), 1.30 (s, 3H), 1.24 (s, 3H), 1.02 (s, 3H). | LCMS (ES, m/z): 507 [M + H]+, Rt 0.658 min. |
| 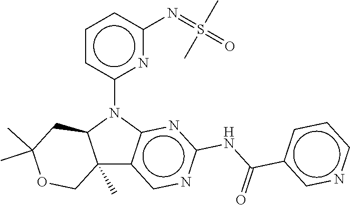 | 405 | 1H NMR (400 MHz, DMSO-d6) δ 11.02 (br, 1H), 9.06 (s, 1H), 8.75-8.73 (m, 1H), 8.28-8.19 (m, 1H), 8.25-8.17 (m, 2H), 7.58-7.48 (m, 2H), 6.32 (d, J = 7.6 Hz, 1H), 4.62-4.59 (m, 1H), 3.87-3.82 (m, 1H), 3.66-3.61 (m, 1H),3.49 (s, 3H), 3.29 (s, 3H), 2.50-2.46 (m, 1H), 1.58-1.51 (m, 1H), 1.27 (s, 3H), 1.24 (s, 3H), 1.02 (s, 3H). | LCMS (ES, m/z): 508 [M + H]+, Rt 1.100 min. |
| 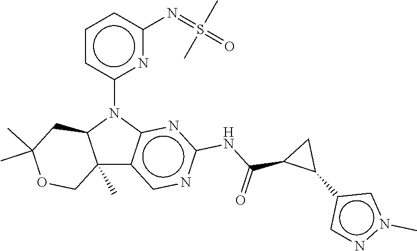 | 406 | 1H NMR (300 MHz, DMSO-d6) δ 10.60 (s, 1H), 8.30 (d, J = 7.8 Hz, 1H), 8.13 (s, 1H), 7.53-7.47 (m, 2H), 7.26 (s, 1H), 6.32 (d, J = 7.8 Hz, 1H), 4.59-4.54 (m, 1H), 3.87-3.83 (m, 1H), 3.75-3.64 (m, 4H), 3.48 (s, 3H), 3.32 (s, 3H), 2.44-2.42 (m, 1H), 2.32-2.11 (m, 2H), 1.55-1.51 (m, 1H), 1.48- 1.40 (m, 1H), 1.27 (s, 3H), 1.22 (s, 3H), 1.19- 1.12 (m, 1H), 1.00 (s, 3H). | LCMS (ES, m/z): 551 [M + H]+, Rt 1.227 min. |
| 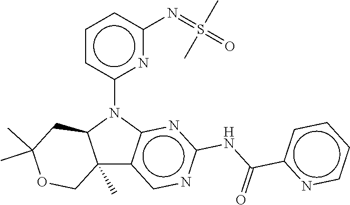 | 407 | 1H NMR (400 MHz, DMSO-d6) δ 10.52 (s, 1H), 8.78-8.76 (m, 1H), 8.40 (d, J = 8.0 Hz, 1H), 8.26- 8.23 (m, 2H), 8.12 (t, J = 7.6 Hz, 1H), 7.76-7.72 (m, 1H), 6.62 (t, J = 8.0 Hz, 1H), 6.37 (d, J = 8.0 Hz, 1H), 4.67-4.60 (m, 1H), 3.93-3.87 (m, 1H), 3.74-3.66 (m, 1H), 3.51 (s, 3H), 3.31 (s, 3H), 2.50-2.47 (m, 1H), 1.63-1.53 (m, 1H), 1.32 (s, 3H), 1.24 (s, 3H), 1.02 (s, 3H). | LCMS (ES, m/z): 508 [M + H]+; RT: 1.207 min. |
| 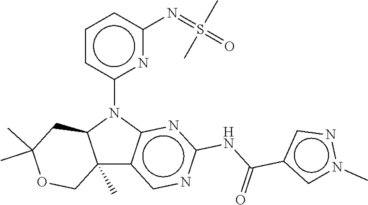 | 408 | 1H NMR (400 MHz, DMSO-d6) δ 10.41 (br, 1H), 8.41-8.36 (m, 2H), 8.20 (s, 1H), 8.08 (s, 1H), 7.55 (t, J = 8.0 Hz, 1H), 6.32 (d, J = 8.0 Hz, 1H), 4.61-4.57 (m, 1H), 3.91-3.88 (m, 4H), 3.70-3.67 (m, 1H), 3.50 (s, 3H), 3.29 (s, 3H), 2.50-2.45 (m, 1H), 1.56-1.51 (m, 1H), 1.30 (s, 3H), 1.24 (s, 3H), 1.02 (s, 3H). | LCMS (ES, m/z): 511 [M + H]+, Rt 1.246 min. |
| 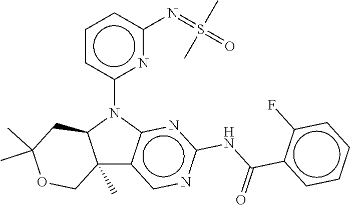 | 409 | 1H NMR (400 MHz, DMSO-d6) δ 10.85 (s, 1H), 8.17 (s, 1H), 7.87 (d, J = 8.0 Hz, 1H), 7.88-7.63 (m, 1H), 7.63-7.56 (m, 1H), 7.43 (t, J = 8.0 Hz, 1H), 7.37-7.17 (m, 2H), 6.30 (d, J = 7.6 Hz, 1H), 4.58-4.52 (m, 1H), 3.87-3.82 (m, 1H), 3.66-3.61 (m, 1H), 3.48 (s, 3H), 3.28 (s, 3H), 2.50-2.01 (m, 1H), 1.54-1.44 (m, 1H), 1.27 (s, 3H), 1.24 (s, 3H), 1.00 (s, 3H). | LCMS (ES, m/z): 525 [M + H]+, Rt 0.673 min. |
| 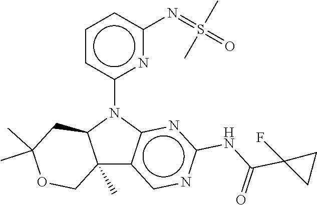 | 410 | 1H NMR (400 MHz, DMSO-d6) δ 10.28 (s, 1H), 8.59-7.87 (m, 2H), 7.54 (t, J = 8.0 Hz, 1H), 6.33 (d, J = 7.6 Hz, 1H), 4.61-4.58 (m, 1H), 3.87-3.82 (m, 1H), 3.66-3.61 (m, 1H), 3.49 (s, 3H), 3.29 (s, 3H), 2.51-2.43 (m, 1H), 1.56-1.51(m, 1H), 1.48- 1.31 (m, 4H), 1.29 (s, 3H), 1.23 (s, 3H), 1.00 (s, 3H). | LCMS (ES, m/z): 489 [M + H]+, Rt 0.662 min. |
| 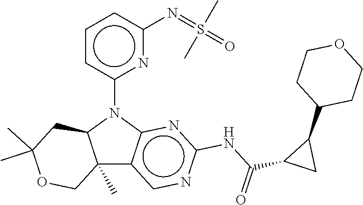 | 411 | 1H NMR (400 MHz, DMSO-d6) δ 10.53 (s, 1H), 8.31 (d, J = 8.0 Hz, 1H), 8.13 (s, 1H), 7.52 (t, J = 8.0 Hz, 1H), 6.32 (d, J = 7.6 Hz, 1H), 4.58-4.54 (m, 1H), 3.88-3.81 (m, 3H), 3.66-3.61 (m, 1H), 3.48 (s, 3H), 3.32-3.22 (m, 5H), 2.51-2.49 (m, 1H), 2.44 (s, 1H), 1.60-1.49 (m, 3H), 1.35-1.14 (m, 9H), 1.04-1.00 (m, 5H), 0.81-0.73 (m, 1H). | LCMS (ES, m/z): 555 [M + H]+, Rt 0.662 min. |
| 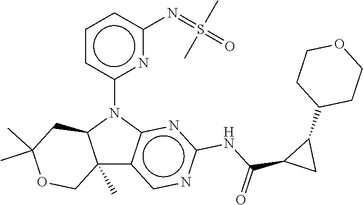 | 412 | 1H NMR (400 MHz, DMSO-d6) δ 10.53 (s, 1H), 8.31 (d, J = 8 Hz, 1H), 8.13 (s, 1H), 7.52 (t, J = 8 Hz, 1H), 6.32 (d, J = 7.6 Hz, 1H), 4.58-4.55 (m, 1H), 3.87-3.81 (m, 3H), 3.68 (d, J = 12 Hz, 1H), 3.48 (s, 3H), 3.32-3.22 (m, 5H), 2.51-2.49 (m, 1H), 2.44 (s, 1H), 1.60-1.50 (m, 3H), 1.34-1.14 (m, 9H), 1.07-0.98 (m, 5H), 0.81-0.74 (m, 1H). | LCMS (ES, m/z): 555 [M + H]+, Rt 0.672 min. |
| 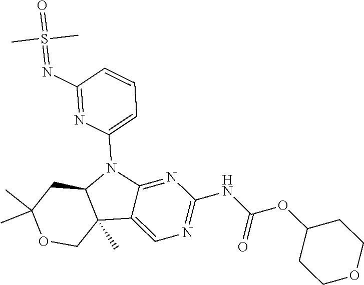 [SMILES: CC1(C)C[C@H]2N(c3cccc(N=S(C)(C)=O)n3)c3nc(NC(=O)OC4CCOCC4)ncc3[C@@]2(C)CO1] | 772 | 1H NMR (400 MHz, DMSO-d6) δ 10.10 (s, 1H), 8.34 (d, J = 8.0 Hz, 1H), 8.10 (s, 1H), 7.52 (t, J = 8.0 Hz, 1H), 6.32 (d, J = 7.6 Hz, 1H), 4.92-4.85 (m, 1H), 4.59-4.55 (m, 1H), 3.90-3.79 (m, 3H), 3.66-3.62 (m, 1H), 3.48-3.41 (m, 5H), 3.29 (s, 3H), 2.50-2.45 (m, 1H), 1.97-1.90 (m, 2H), 1.68- 1.42 (m, 3H), 1.27 (s, 3H), 1.24 (s, 3H), 1.00 (s, 3H). | LCMS (ES, m/z): 531 [M + H]+, Rt 1.151 min. |
| 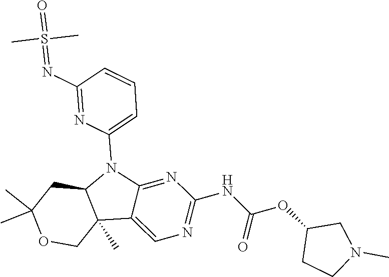 | 773 | 1H NMR (400 MHz, DMSO-d6) δ 10.06 (s, 1H), 8.27 (d, J = 8.0 Hz, 1H), 8.03 (s, 1H), 7.45 (t, J = 8.0 Hz, 1H), 6.25 (d, J = 7.6 Hz, 1H), 5.05-5.04 (m, 1H), 4.50-4.48 (m, 1H), 4.02 (t, J = 6.0 Hz, 1H), 3.80-3.77 (m, 1H), 3.61-3.58 (m, 2H), 3.42 (s, 3H), 3.22 (s, 3H), 2.60-2.55 (m, 2H), 2.18 (s, 3H), 2.14-2.08 (m, 1H), 1.70-1.67 (m, 2H), 1.47- 1.41 (m, 1H), 1.20 (s, 3H), 1.16 (s, 3H), 0.93 (s, 3H). | LCMS (ES, m/z): 530 [M + H]+, Rt 0.895 min |

### Phosphorylated Cdc2 MSD Electrochemiluminescence Assay

The effect of Wee1 inhibitors on cellular phosphorylation of the Wee1 substrate Cdc2 was determined using the following protocol:

A total of 40,000 A427 cells in 100 uL culture medium (1640 medium+10% Fetal bovine serum+1% Penicillin-Streptomycin) were plated in 96-well cell culture plates. 3-fold serial dilutions of test compounds were prepared in completed PBS at 25× final concentration. After 20 hours, the plates were removed and 4 μL of each were added to the cells and incubated with shaking for 30 minutes at 37° C., 5% CO₂. Each concentration was tested in duplicate. After a total 6 hours incubation, plates were removed and centrifuged for 5 minutes and 100 uL of supernatant from each well was removed. Cells were washed with 200 μL PBS and lysed with 50 μL MSD lysis buffer (Meso Scale Diagnostics) supplied with 1× complete ULTRA cocktail inhibitor (Roche).

To detect phosphorylation of Cdc2 Y15, 30 μL of capture antibody solution (CST catalog #9116S 1:200) was added into each well of the MSD plate (MSD catalog #L15XB-6), sealed and incubated at 4° C. with shaking (450 rpm) overnight. The antibody solution was removed, wells blocked with BSA solution and plates washed three times with PBST, followed by addition of 30 μl of cell lysate per well. After 2 hours of incubation, plates were washed three times with PBST. 30 μL of 1× detection antibody solution (CST catalog #4539S 1:200) was then added to each well and incubated for 1 hour. Plates were washed three times with PBST and 30 μL of 1× secondary antibody solution (MSD, catalog #:R32AB-1, 1:5000) was added to each well and incubated for 1 hour. Plates were washed three times with PBST and 150 μL of 1× Read Buffer T (MSD, catalog #R92TC-1) was added to each well of the MSD plate. The electrochemiluminescence signal was measured on a MESO SECTOR S600 plate reader. The percentage of remaining phosphorylated Cdc2 signal was calculated following the equation below.

$$\% Inhibition = 100 \times \frac{{R}_{HC} - {R}_{cpds}}{{R}_{HC} - {R}_{LC}}$$

HC (high control): Cells treated with DMSO
Cpds: Cells treated with test compounds
LC (low control): Cells treated with positive control Wee1 inhibitor

### Wee1 Kinase Biochemical Assay Protocol

Wee1 kinase domain was purchased from Carna (catalog #05-177). Wee1 kinase activity was determined with Poly(Lys,Tyr 4:1) hydrobromide as a substrate (Sigma-Aldrich) and by measuring ADP production using the ADP-Glo Kinase Assay kit (Promega) following the manufacturer's instructions. The kinase reaction was performed using the following conditions:

Buffer: 40 mM Tris-HCl, 20 mM magnesium chloride, supplemented with 0.1 mg/mL bovine serum albumin and 2 mM DTT. The final reaction mix contained 1 nM Wee1 enzyme, 15 uM ATP, and 2 ng/mL Poly(Lys,Tyr 4:1) hydrobromide substrate. The reaction time was 4 hours at 25° C.

The ADP-Glo signal was measured using the Envision plate reader and the percentage inhibition of kinase activity calculated for each inhibitor tested. Percent inhibition of Wee1 kinase activity was calculated based on the following formula.

$$Inhibition ( \% ) = 100 \% \times ( 1 - \frac{{S}_{Sample} - {S}_{Low Ctrl}}{{S}_{High Ctrl} - {S}_{Low Ctrl}} )$$

S_{Sample}: the signal of compounds
S_{High Ctrl}: the signal of high control (DMSO)
S_{Low Ctrl}: the signal of low control (positive control Wee1 inhibitor at saturating concentration)

The WEE1 IC₅₀ ranges are as follows:
A: IC₅₀<10 nM; B: 10 nM≤IC₅₀<100 nM; C: 100 nM≤IC₅₀<1000 nM; D: IC₅₀≥1000 nM.

The A427 pCDC2 IC₅₀ ranges are as follows:
A: IC₅₀<100 nM; B: 100 nM≤IC₅₀<1000 nM; C: 1000 nM≤IC₅₀<10000 nM; D: IC₅₀≥10000 nM.

TABLE A
Biological Data Obtained
in the above assays.

| | WEE1 | A427 |
|---|---|---|
| | kinase | pCDC2 |
| Ex. | ADP-Glo | MSD |
| No. | IC50 | IC50 |
| 1 | B | NA |
| 2 | A | B |
| 3 | C | NA |
| 4 | A | B |
| 5 | A | B |
| 6 | B | NA |
| 7 | B | NA |
| 8 | A | B |
| 9 | A | B |
| 10 | B | NA |
| 11 | A | B |
| 12 | C | NA |
| 13 | B | NA |
| 14 | A | B |
| 15 | B | NA |
| 16 | A | B |
| 17 | C | NA |
| 18 | A | B |
| 19 | C | NA |
| 20 | A | B |
| 21 | C | NA |
| 22 | A | B |
| 23 | C | NA |
| 24 | A | B |
| 25 | B | NA |
| 26 | A | B |
| 27 | D | NA |
| 28 | A | NA |
| 29 | B | NA |
| 30 | A | B |
| 31 | C | NA |
| 32 | A | B |
| 33 | C | NA |
| 34 | A | B |
| 35 | A | B |
| 36 | B | NA |
| 37 | C | NA |
| 38 | A | A |
| 39 | C | NA |
| 40 | A | A |
| 41 | A | A |
| 42 | B | NA |
| 43 | B | NA |
| 44 | A | A |
| 45 | A | B |
| 46 | B | NA |
| 47 | C | NA |
| 48 | A | A |
| 49 | A | B |
| 50 | B | NA |
| 51 | D | NA |
| 52 | A | A |
| 53 | D | NA |
| 54 | A | A |
| 55 | D | NA |
| 56 | A | B |
| 57 | B | NA |
| 58 | D | NA |
| 59 | A | NA |
| 60 | A | NA |
| 61 | A | A |
| 62 | B | NA |
| 63 | A | B |
| 64 | B | NA |
| 65 | A | B |
| 66 | C | NA |
| 67 | C | NA |
| 68 | A | B |
| 69 | A | A |
| 70 | B | NA |
| 71 | C | NA |
| 72 | A | B |
| 73 | A | B |
| 74 | B | NA |
| 75 | D | NA |
| 76 | A | B |
| 77 | C | NA |
| 78 | A | B |
| 79 | C | NA |
| 80 | A | B |
| 81 | D | NA |
| 82 | A | B |
| 83 | D | NA |
| 84 | A | B |
| 85 | A | B |
| 86 | C | NA |
| 87 | A | B |
| 88 | A | B |
| 89 | D | NA |
| 90 | NA | NA |
| 91 | C | NA |
| 92 | A | B |
| 93 | B | NA |
| 94 | A | B |
| 95 | D | NA |
| 96 | A | B |
| 97 | C | NA |
| 98 | A | NA |
| 99 | C | NA |
| 100 | A | A |
| 101 | C | C |
| 102 | A | B |
| 103 | B | NA |
| 104 | A | A |
| 105 | B | NA |
| 106 | A | A |
| 107 | A | A |
| 108 | B | NA |
| 109 | A | A |
| 110 | B | NA |
| 111 | B | NA |
| 112 | A | A |
| 113 | B | NA |
| 114 | A | A |
| 115 | B | NA |
| 116 | C | NA |
| 117 | A | A |
| 118 | D | NA |
| 119 | A | B |
| 120 | A | A |
| 121 | C | NA |
| 122 | A | A |
| 123 | C | NA |
| 124 | A | A |
| 125 | B | NA |
| 126 | A | A |
| 127 | A | A |
| 128 | C | NA |
| 129 | A | A |
| 130 | C | NA |
| 131 | A | A |
| 132 | B | NA |
| 133 | A | B |
| 134 | C | NA |
| 135 | A | A |
| 136 | B | NA |
| 137 | A | A |
| 138 | B | NA |
| 139 | C | NA |
| 140 | A | A |
| 141 | A | A |
| 142 | B | NA |
| 143 | A | B |
| 144 | C | NA |
| 145 | A | A |
| 146 | B | NA |
| 147 | A | A |
| 148 | B | NA |
| 149 | A | B |
| 150 | B | NA |
| 151 | C | NA |
| 152 | A | B |
| 153 | A | A |
| 154 | C | NA |
| 155 | A | B |
| 156 | B | NA |
| 157 | A | A |
| 158 | C | NA |
| 159 | A | A |
| 160 | C | NA |
| 161 | C | NA |
| 162 | A | A |
| 163 | A | A |
| 164 | B | NA |
| 165 | A | A |
| 166 | B | NA |
| 167 | B | NA |
| 168 | A | A |
| 169 | A | A |
| 170 | B | NA |
| 171 | A | B |
| 172 | B | NA |
| 173 | A | A |
| 174 | B | NA |
| 175 | C | NA |
| 176 | A | B |
| 177 | A | B |
| 178 | C | NA |
| 179 | C | NA |
| 180 | A | B |
| 181 | A | A |
| 182 | C | NA |
| 183 | A | B |
| 184 | A | A |
| 185 | C | NA |
| 186 | C | NA |
| 187 | D | NA |
| 188 | A | A |
| 189 | C | NA |
| 190 | A | B |
| 191 | C | NA |
| 192 | A | B |
| 193 | C | NA |
| 194 | A | B |
| 195 | C | NA |
| 196 | A | B |
| 197 | C | NA |
| 198 | A | B |
| 199 | A | B |
| 200 | B | NA |
| 201 | A | B |
| 202 | A | B |
| 203 | C | NA |
| 204 | A | B |
| 205 | A | A |
| 206 | C | NA |
| 207 | A | B |
| 208 | B | NA |
| 209 | B | NA |
| 210 | A | B |
| 211 | B | NA |
| 212 | A | B |
| 213 | B | NA |
| 214 | A | B |
| 215 | C | NA |
| 216 | A | B |
| 217 | A | B |
| 218 | B | NA |
| 219 | A | B |
| 220 | C | NA |
| 221 | A | A |
| 223 | A | B |
| 225 | A | C |
| 227 | A | C |
| 229 | B | NA |
| 232 | A | C |
| 233 | A | B |
| 234 | B | NA |
| 235 | A | B |
| 236 | B | NA |
| 237 | A | A |
| 238 | A | B |
| 239 | A | NA |
| 240 | C | NA |
| 241 | A | B |
| 242 | B | NA |
| 243 | A | A |
| 244 | B | NA |
| 245 | A | B |
| 246 | B | C |
| 247 | A | B |
| 248 | C | NA |
| 249 | A | B |
| 250 | B | NA |
| 251 | A | B |
| 252 | C | NA |
| 253 | A | B |
| 254 | B | NA |
| 255 | A | B |
| 256 | B | NA |
| 257 | A | B |
| 258 | C | NA |
| 259 | A | B |
| 260 | A | B |
| 261 | B | NA |
| 262 | C | B |
| 263 | A | A |
| 264 | B | B |
| 265 | A | A |
| 266 | B | NA |
| 267 | A | B |
| 268 | B | NA |
| 269 | A | B |
| 270 | B | NA |
| 271 | A | B |
| 272 | B | NA |
| 273 | A | B |
| 274 | B | NA |
| 275 | A | B |
| 276 | B | NA |
| 277 | A | B |
| 278 | B | NA |
| 279 | A | B |
| 280 | C | NA |
| 281 | A | B |
| 282 | C | NA |
| 283 | A | B |
| 284 | B | NA |
| 285 | A | B |
| 286 | C | NA |
| 287 | A | B |
| 288 | B | NA |
| 289 | A | B |
| 290 | A | NA |
| 291 | A | B |
| 292 | B | NA |
| 293 | A | B |
| 294 | B | NA |
| 295 | A | B |
| 296 | A | NA |
| 297 | A | B |
| 298 | B | NA |
| 299 | A | B |
| 300 | C | NA |
| 301 | A | C |
| 302 | C | NA |
| 303 | A | B |
| 304 | B | NA |
| 305 | A | B |
| 306 | A | B |
| 307 | A | NA |
| 308 | B | NA |
| 309 | B | NA |
| 310 | A | B |
| 311 | A | B |
| 312 | B | NA |
| 313 | A | B |
| 314 | B | NA |
| 315 | A | B |
| 316 | B | NA |
| 317 | A | B |
| 318 | B | NA |
| 319 | A | B |
| 320 | C | NA |
| 321 | A | B |
| 322 | C | NA |
| 323 | A | B |
| 324 | C | NA |
| 325 | A | B |
| 326 | B | NA |
| 327 | A | B |
| 328 | C | NA |
| 329 | A | B |
| 330 | B | NA |
| 331 | A | B |
| 332 | B | NA |
| 333 | A | B |
| 334 | C | NA |
| 335 | A | A |
| 336 | B | NA |
| 337 | A | B |
| 338 | B | NA |
| 339 | A | B |
| 340 | C | NA |
| 341 | A | B |
| 342 | C | NA |
| 343 | A | B |
| 344 | B | NA |
| 345 | A | B |
| 346 | B | NA |
| 347 | A | B |
| 348 | B | NA |
| 349 | A | B |
| 350 | B | NA |
| 351 | A | A |
| 352 | A | B |
| 353 | B | NA |
| 354 | B | NA |
| 355 | A | B |
| 356 | B | NA |
| 357 | C | NA |
| 359 | B | NA |
| 360 | A | B |
| 361 | A | NA |
| 362 | A | NA |
| 363 | A | B |
| 364 | A | B |
| 365 | A | C |
| 366 | A | C |
| 367 | A | B |
| 368 | B | NA |
| 369 | A | B |
| 370 | A | B |
| 371 | A | B |
| 372 | A | B |
| 373 | A | B |
| 374 | A | B |
| 375 | A | B |
| 376 | A | B |
| 377 | A | C |
| 378 | A | B |
| 379 | A | B |
| 380 | A | B |
| 381 | A | NA |
| 382 | B | NA |
| 383 | A | NA |
| 384 | A | NA |
| 385 | A | NA |
| 386 | A | B |
| 387 | A | C |
| 388 | A | B |
| 389 | A | C |
| 390 | A | B |
| 391 | A | B |
| 392 | A | C |
| 393 | A | C |
| 394 | A | C |
| 395 | A | B |
| 396 | A | B |
| 397 | A | B |
| 398 | B | NA |
| 399 | A | B |
| 400 | A | B |
| 401 | A | B |
| 402 | B | C |
| 403 | A | B |
| 404 | B | NA |
| 405 | B | NA |
| 406 | A | B |
| 407 | B | NA |
| 408 | A | C |
| 409 | C | NA |
| 410 | B | NA |
| 411 | A | C |
| 412 | A | D |
| 413 | A | B |
| 414 | C | NA |
| 415 | C | NA |
| 416 | A | B |
| 417 | A | A |
| 418 | B | NA |
| 419 | A | A |
| 420 | B | NA |
| 421 | A | A |
| 422 | B | NA |
| 423 | A | B |
| 424 | B | NA |
| 425 | A | B |
| 426 | B | NA |
| 427 | A | B |
| 428 | C | NA |
| 429 | A | B |
| 430 | B | NA |
| 431 | A | B |
| 432 | C | NA |
| 433 | A | B |
| 434 | B | NA |
| 435 | A | B |
| 436 | B | NA |
| 437 | A | B |
| 438 | C | NA |
| 439 | A | B |
| 440 | B | NA |
| 441 | A | B |
| 442 | C | NA |
| 443 | A | B |
| 444 | C | NA |
| 445 | C | NA |
| 446 | A | B |
| 447 | C | NA |
| 448 | A | B |
| 449 | B | NA |
| 450 | A | B |
| 451 | C | NA |
| 452 | A | B |
| 453 | C | NA |
| 454 | A | B |
| 455 | D | NA |
| 456 | B | NA |
| 457 | D | NA |
| 458 | A | C |
| 459 | C | NA |
| 460 | A | B |
| 461 | D | NA |
| 462 | B | NA |
| 463 | C | NA |
| 464 | A | B |
| 465 | C | NA |
| 466 | A | C |
| 467 | D | NA |
| 468 | A | C |
| 469 | C | NA |
| 470 | A | B |
| 471 | C | NA |
| 472 | A | B |
| 473 | A | B |
| 474 | C | NA |
| 475 | C | NA |
| 476 | A | B |
| 477 | C | NA |
| 478 | B | NA |
| 479 | A | B |
| 480 | C | NA |
| 481 | A | B |
| 482 | C | NA |
| 483 | A | A |
| 484 | A | A |
| 485 | A | B |
| 486 | A | A |
| 487 | A | B |
| 488 | A | B |
| 490 | A | B |
| 491 | A | B |
| 492 | A | A |
| 493 | A | B |
| 494 | A | B |
| 495 | A | C |
| 496 | A | B |
| 497 | A | B |
| 498 | A | B |
| 499 | A | A |
| 500 | A | A |
| 501 | A | A |
| 502 | A | A |
| 503 | A | A |
| 504 | A | B |
| 505 | A | B |
| 506 | A | B |
| 507 | A | NA |
| 508 | A | NA |
| 509 | A | NA |
| 510 | A | NA |
| 511 | A | NA |
| 512 | C | NA |
| 513 | A | NA |
| 514 | A | B |
| 515 | A | NA |
| 516 | A | NA |
| 517 | A | B |
| 518 | A | B |
| 519 | A | B |
| 520 | A | C |
| 521 | C | NA |
| 522 | A | B |
| 523 | A | B |
| 524 | C | NA |
| 525 | C | NA |
| 526 | A | B |
| 527 | B | NA |
| 528 | A | NA |
| 529 | A | B |
| 530 | B | NA |
| 531 | A | B |
| 532 | C | NA |
| 533 | A | B |
| 534 | C | NA |
| 535 | A | B |
| 536 | B | NA |
| 537 | A | B |
| 538 | B | NA |
| 539 | A | B |
| 540 | B | NA |
| 541 | C | NA |
| 542 | A | B |
| 543 | A | B |
| 544 | C | NA |
| 545 | A | B |
| 546 | C | NA |
| 547 | B | NA |
| 548 | D | NA |
| 549 | A | A |
| 550 | C | NA |
| 551 | A | A |
| 552 | C | NA |
| 553 | A | B |
| 554 | C | NA |
| 555 | B | NA |
| 556 | A | B |
| 557 | A | NA |
| 558 | C | NA |
| 559 | D | NA |
| 560 | B | NA |
| 561 | D | NA |
| 562 | A | B |
| 563 | C | NA |
| 564 | A | A |
| 565 | A | B |
| 566 | D | NA |
| 567 | A | A |
| 568 | B | NA |
| 569 | A | A |
| 570 | C | NA |
| 571 | A | NA |
| 572 | A | B |
| 573 | C | NA |
| 574 | B | B |
| 575 | A | A |
| 576 | C | NA |
| 577 | A | A |
| 578 | C | NA |
| 579 | A | B |
| 580 | C | NA |
| 581 | A | B |
| 582 | C | NA |
| 583 | A | B |
| 584 | C | NA |
| 585 | A | B |
| 586 | B | NA |
| 587 | A | B |
| 588 | B | NA |
| 589 | A | B |
| 590 | B | NA |
| 591 | A | B |
| 592 | B | NA |
| 593 | A | B |
| 594 | C | NA |
| 595 | A | B |
| 596 | B | NA |
| 597 | A | B |
| 598 | C | NA |
| 599 | A | B |
| 600 | C | NA |
| 601 | A | NA |
| 602 | A | B |
| 603 | B | NA |
| 604 | B | NA |
| 605 | A | B |
| 606 | B | NA |
| 607 | A | B |
| 608 | A | B |
| 609 | B | NA |
| 610 | D | NA |
| 611 | A | C |
| 612 | C | NA |
| 613 | A | A |
| 614 | C | NA |
| 615 | A | A |
| 616 | A | B |
| 617 | C | NA |
| 618 | A | A |
| 619 | B | NA |
| 620 | A | A |
| 621 | C | NA |
| 622 | A | B |
| 623 | B | NA |
| 624 | A | B |
| 625 | B | NA |
| 626 | A | B |
| 627 | B | NA |
| 628 | C | NA |
| 629 | A | B |
| 630 | C | NA |
| 631 | A | B |
| 632 | A | B |
| 633 | B | NA |
| 634 | A | B |
| 635 | B | NA |
| 636 | A | B |
| 637 | B | NA |
| 638 | A | B |
| 639 | B | NA |
| 640 | A | B |
| 641 | C | NA |
| 642 | A | B |
| 643 | B | NA |
| 644 | A | B |
| 645 | C | NA |
| 646 | A | B |
| 647 | B | NA |
| 650 | B | NA |
| 651 | A | B |
| 652 | A | B |
| 653 | B | NA |
| 654 | A | B |
| 655 | B | NA |
| 656 | B | NA |
| 657 | A | B |
| 658 | A | B |
| 659 | B | NA |
| 660 | A | B |
| 661 | B | NA |
| 662 | A | B |
| 663 | B | NA |
| 664 | A | B |
| 665 | B | NA |
| 666 | A | B |
| 667 | B | NA |
| 668 | A | B |
| 669 | B | NA |
| 670 | A | B |
| 671 | B | NA |
| 672 | A | B |
| 673 | B | NA |
| 674 | B | NA |
| 675 | A | B |
| 676 | A | B |
| 677 | B | NA |
| 680 | A | B |
| 681 | B | NA |
| 682 | A | B |
| 683 | C | NA |
| 684 | A | B |
| 685 | B | NA |
| 686 | A | B |
| 687 | C | NA |
| 688 | A | A |
| 689 | C | NA |
| 690 | A | B |
| 691 | C | NA |
| 692 | A | B |
| 693 | C | NA |
| 694 | A | B |
| 695 | B | NA |
| 696 | A | B |
| 697 | B | NA |
| 698 | A | B |
| 699 | C | NA |
| 700 | A | B |
| 701 | B | NA |
| 702 | A | B |
| 703 | A | B |
| 704 | C | NA |
| 705 | A | B |
| 706 | B | NA |
| 707 | A | B |
| 708 | B | NA |
| 709 | D | NA |
| 710 | A | C |
| 711 | C | NA |
| 712 | A | C |
| 713 | A | B |
| 714 | B | NA |
| 715 | C | NA |
| 716 | A | C |
| 717 | A | B |
| 719 | A | C |
| 720 | A | B |
| 721 | A | B |
| 722 | A | B |
| 723 | B | NA |
| 725 | A | B |
| 726 | A | B |
| 727 | A | A |
| 728 | A | B |
| 729 | A | B |
| 730 | A | B |
| 731 | A | B |
| 732 | B | NA |
| 733 | B | NA |
| 734 | A | A |
| 735 | B | NA |
| 736 | C | NA |
| 737 | A | B |
| 738 | B | NA |
| 739 | A | C |
| 742 | A | C |
| 744 | A | C |
| 746 | A | B |
| 748 | A | B |
| 749 | A | B |
| 751 | A | B |
| 752 | A | B |
| 753 | A | B |
| 754 | A | A |
| 755 | A | B |
| 756 | A | B |
| 757 | A | B |
| 758 | A | B |
| 759 | A | B |
| 760 | A | B |
| 761 | B | NA |
| 762 | B | NA |
| 763 | B | NA |
| 764 | A | B |
| 765 | A | B |
| 766 | A | B |
| 767 | A | B |
| 768 | A | B |
| 769 | C | NA |
| 770 | A | B |
| 771 | A | B |
| 772 | A | B |
| 773 | B | NA |
| 774 | A | B |
| 775 | A | B |
| 776 | A | B |
| 777 | A | C |
| 778 | A | B |
| 779 | A | A |
| 780 | A | A |
| 781 | A | B |
| 782 | A | A |
| 783 | A | A |
| 784 | A | B |
| 785 | A | NA |
| 786 | A | NA |
| 787 | A | NA |
| 788 | A | NA |
| 789 | A | NA |
| 790 | A | NA |
| 791 | A | NA |
| 792 | A | NA |
| 793 | A | NA |
| 794 | A | NA |
| 795 | A | NA |
| 796 | A | NA |
| 797 | A | NA |
| 798 | A | NA |
| 799 | A | C |
| 800 | A | NA |
| 801 | A | NA |
| 802 | A | NA |
| 803 | A | NA |
| 804 | A | NA |
| 805 | A | NA |
| 806 | A | NA |
| 807 | A | NA |
| 808 | A | NA |
| 809 | A | NA |
| 810 | A | B |
| 811 | A | NA |
| 812 | A | B |
| 813 | A | NA |
| 814 | A | B |
| 815 | A | NA |
| 816 | A | NA |
| 817 | A | NA |
| 818 | A | NA |
| 819 | A | NA |
| 820 | A | NA |
| 823 | A | NA |
| 824 | A | NA |
| 825 | A | NA |
| 826 | A | NA |
| 827 | A | NA |
| 828 | A | NA |
| 829 | A | NA |
| 830 | A | NA |
| 831 | A | NA |
| 832 | A | NA |

### NUMBERED EMBODIMENTS

1. A compound of Formula (I):

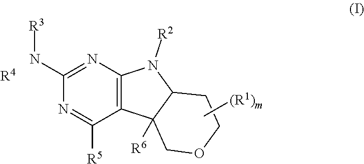

or a pharmaceutically acceptable salt thereof, wherein:
each R¹ is independently C1-C6 alkyl;
m is 0, 1, or 2;
R² is hydrogen, C1-C6 alkyl, phenyl, 5-10 membered heteroaryl containing one or more ring atom selected from N, O, or S, or 5-10 membered heterocyclyl containing one or more ring atom selected from N, O, S, C(O), N(O), S(O), or S(O)₂, wherein each of phenyl, 5-10 membered heteroaryl, and 5-10 membered heterocyclyl is optionally substituted with 1-3 substituents each independently selected from R^{A};
each R^{A} is independently selected from: halogen; C1-C6 haloalkyl, optionally substituted with NR^{B}R^{C}; hydroxyl; cyano; NR^{B}R^{C}; C(═O)NR^{B}R^{C}; N═S(O)(R^{J})₂, wherein each R^{J} is methyl or both taken together with the S atom to which they are attached form a 5 membered ring; S(O)₂C1-C6 alkyl; S(O)(═NR^{B})C1-C6 alkyl; P(O)(C1-C6 alkyl)₂; C1-C6 alkyl, optionally substituted with hydroxyl, or NR^{B}R^{C}; C1-C6 alkoxy; C3-C6 cycloalkyl, optionally substituted with hydroxyl or NR^{B}R^{C}; and 4-6 membered heterocyclyl containing one or more ring atom selected from N, O, S, C(O), N(O), S(O), or S(O)₂, optionally substituted with 1-2 substituents independently selected from halogen, hydroxyl, and C1-C6 alkyl;
each R^{B} and R^{C} are independently hydrogen or C1-C6 alkyl;
R³ is hydrogen or C1-C6 alkyl;
R⁴ is
(i) hydrogen;
(ii) phenyl, optionally substituted with 1 or 2 substituents each independently selected from the group consisting of:
   halogen; cyano; SO₂(C1-C6 alkyl); C1-C6 haloalkyl; C1-C6 deuteroalkyl; C1-C6 alkyl, optionally substituted with 1 or 2 substituents each independently selected from: —NR^{B}R^{C} and —CO₂H; (C1-C6 alkyl)ₙ-C(═O)NR^{E}R^{F}; C3-C6 cycloalkyl, optionally substituted with C1-C6 alkyl; 5-6 membered heteroaryl containing one or more ring atom selected from N, O, or S, optionally substituted with C1-C6 alkyl; 4-12 membered heterocyclyloxy containing one or more ring atom selected from N, O, S, C(O), N(O), S(O), or S(O)₂, optionally substituted with 1 or 2 independently selected R^{G}; and 4-12 membered heterocyclyl containing one or more ring atom selected from N, O, S, C(O), N(O), S(O), or S(O)₂, optionally substituted with 1, 2, or 3 independently selected R^{G};
(iii) 9-12 membered heterocyclyl containing one or more ring atom selected from N, O, S, C(O), N(O), S(O), or S(O)₂, optionally substituted with 1-4 independently selected C1-C6 alkyl, or halogen;
(iv) 5-10 membered heteroaryl containing one or more ring atom selected from N, O, or S, optionally substituted with 1 or 2 substituents independently selected from the group consisting of C1-C6 alkyl; cyano; halogen; C1-C6 haloalkyl; C1-C6 deuteroalkyl; C1-C6 alkoxy; C3-C6 cycloalkyl; 5-6 membered heteroaryl containing one or more ring atom selected from N, O, or S, optionally substituted with C1-C6 alkyl; 4-12 membered heterocyclyloxy containing one or more ring atom selected from N, O, S, C(O), N(O), S(O), or S(O)₂, optionally substituted with 1 or 2 independently selected R^{G}; and 4-12 membered heterocyclyl containing one or more ring atom selected from N, O, S, C(O), N(O), S(O), or S(O)₂, optionally substituted with 1 or 2 C1-C6 alkyl, C1-C6 alkoxy, amino, or 4-12 membered heterocyclyl containing one or more ring atom selected from N, O, S, C(O), N(O), S(O), or S(O)₂;
(v) C3-C6 cycloalkyl; or
(vi) C(O)—R^{I};
n is 0 or 1;
each R^{E} and R^{F} are independently hydrogen or C1-C6 alkyl; or
R^{E} and R^{F}, together with the nitrogen atom to which they are attached, form a 4-8 membered heterocyclyl containing one or more ring atom selected from N, O, S, C(O), N(O), S(O), or S(O)₂, optionally substituted with C1-C6 alkyl;
each R^{G} is independently halogen, cyano; C1-C6 alkyl, C1-C6 deuteroalkyl, NR^{B}R^{C}, or ═NR^{H};
R^{H} is hydrogen or C1-C6 alkyl;
R^{I} is selected from the group consisting of C1-C6 alkyl; phenyl, optionally substituted with 1 to 3 halogen; 5-6 membered heteroaryl containing one or more ring atom selected from N, O, or S, optionally substituted with 1 to 3 C1-C6 alkyl; C3-C6 cycloalkyl, optionally substituted with 1 substituent selected from the group consisting of: halogen, phenyl, 5-6 membered heterocyclyl containing one or more ring atom selected from N, O, S, C(O), N(O), S(O), or S(O)₂ and optionally substituted with 1 to 3 C1-C6 alkyl, and 5-6 membered heteroaryl containing one or more ring atom selected from N, O, or S, optionally substituted with 1 to 3 C1-C6 alkyl; and 4-12 membered heterocyclyloxy containing one or more ring atom selected from N, O, S, C(O), N(O), S(O), or S(O)₂, optionally substituted with 1 or 2 C1-C6 alkyl;
R⁵ is hydrogen, halogen, or C1-C6 alkyl; and
R⁶ is hydrogen or C1-C6 alkyl.

2. A compound of Formula (I):

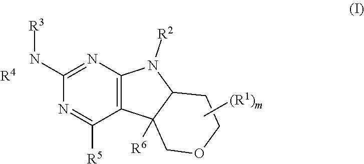

or a pharmaceutically acceptable salt thereof, wherein:
each R¹ is independently C1-C6 alkyl;
m is 0, 1, or 2;
R² is hydrogen, C1-C6 alkyl, phenyl, 5-10 membered heteroaryl containing one or more ring atom selected from N, O, or S, or 5-10 membered heterocyclyl containing one or more ring atom selected from N, O, S, C(O), N(O), S(O), or S(O)₂, wherein the phenyl, 5-10 membered heteroaryl, and 5-10 membered heterocyclyl are each optionally substituted with 1-3 substituents each independently selected from R^{A};
each R^{A} is independently selected from: halogen, cyano, —NR^{B}R^{C}, —C(═O)NR^{B}R^{C}, —N═S(O)Me)₂, C1-C6 alkyl optionally substituted with hydroxyl or —NR^{B}R^{C}; C3-C6 cycloalkyl optionally substituted with —NR^{B}R^{C}; and 4-6 membered heterocyclyl containing one or more ring atom selected from N, O, S, C(O), N(O), S(O), or S(O)₂ optionally substituted with 1-2 substituents independently selected from halogen and C1-C6 alkyl;
each R^{B} and R^{C} are independently hydrogen or C1-C6 alkyl;
R³ is hydrogen or C1-C6 alkyl;
R⁴ is
(i) phenyl optionally substituted with 1 or 2 substituents each independently selected from the group consisting of: halogen, cyano, —SO₂(C1-C6 alkyl), C1-C6 haloalkyl, C1-C6 deuteroalkyl, C1-C6 alkyl optionally substituted with 1 or 2 substituents each independently selected from: —NR^{B}R^{C} and —CO₂H; —(C1-C6 alkyl)ₙ-C(═O)NR^{E}R^{F}, C3-C6 cycloalkyl optionally substituted with C1-C6 alkyl; 5-6 membered heteroaryl containing one or more ring atom selected from N, O, or S optionally substituted with C1-C6 alkyl; 4-12 membered heterocyclyloxy containing one or more ring atom selected from N, O, S, C(O), N(O), S(O), or S(O)₂ optionally substituted with 1 or 2 independently selected R^{G}; and 4-12 membered heterocyclyl containing one or more ring atom selected from N, O, S, C(O), N(O), S(O), or S(O)₂ optionally substituted with 1 or 2 independently selected R^{G};
(ii) 9-12 membered heterocyclyl containing one or more ring atom selected from N, O, S, C(O), N(O), S(O), or S(O)₂ optionally substituted with 1-3 independently selected C1-C6 alkyl;
(iii) 5-10 membered heteroaryl containing one or more ring atom selected from N, O, or S optionally substituted with 1 or 2 substituents independently selected from the group consisting of C1-C6 alkyl, C1-C6 haloalkyl, C1-C6 deuteroalkyl, C3-C6 cycloalkyl, 5-6 membered heteroaryl containing one or more ring atom selected from N, O, or S optionally substituted with C1-C6 alkyl; 4-12 membered heterocyclyloxy containing one or more ring atom selected from N, O, S, C(O), N(O), S(O), or S(O)₂ optionally substituted with 1 or 2 independently selected R^{G}; and 4-12 membered heterocyclyl containing one or more ring atom selected from N, O, S, C(O), N(O), S(O), or S(O)₂ optionally substituted with 1 or 2 C1-C6 alkyl or amino;
(iv) C3-C6 cycloalkyl; or
(v) C(O)—R^{I};
n is 0 or 1;
each R^{E} and R^{F} are independently hydrogen or C1-C6 alkyl; or
R^{E} and R^{F}, together with the nitrogen atom to which they are attached, form a 4-8 membered heterocyclyl containing one or more ring atom selected from N, O, S, C(O), N(O), S(O), or S(O)₂ optionally substituted with C1-C6 alkyl;
each R^{G} is independently halogen, C1-C6 alkyl, C1-C6 deuteroalkyl, —NR^{B}R^{C}, or ═NR^{H};
R^{H} is hydrogen or C1-C6 alkyl;
R^{I} is C1-C6 alkyl, phenyl optionally substituted with 1 to 3 halogen; 5-6 membered heteroaryl containing one or more ring atom selected from N, O, or S optionally substituted with 1 to 3 C1-C6 alkyl; C3-C6 cycloalkyl optionally substituted with 1 substituent selected from the group consisting of: halogen, phenyl, and 5-6 membered heteroaryl containing one or more ring atom selected from N, O, or S optionally substituted with 1 to 3 C1-C6 alkyl;
R⁵ is hydrogen, halogen, or C1-C6 alkyl; and
R⁶ is hydrogen or C1-C6 alkyl.

3. The compound of embodiment 1 or 2, wherein m is 2.

4. The compound of any one of embodiments 1 to 3, wherein each R¹ is methyl.

5. The compound of any one of embodiments 1-4, wherein two independently selected R¹ groups are geminal, and the carbon atom to which they are attached is adjacent to the ring oxygen.

6. The compound according to any one of embodiments 1-5, wherein R⁵ is hydrogen.

7. The compound according to anyone of embodiments 1-6, wherein R⁶ is methyl.

8. The compound according to anyone of embodiments 1-7 having the following structure:

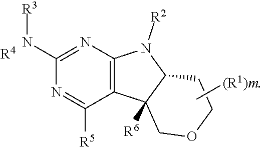

9. The compound of any one of embodiments 1-8, wherein R² is phenyl optionally substituted with 1-3 independently selected R^{A}.

10. The compound of any one of embodiments 1-9, wherein R² is a 5-10 membered heteroaryl containing one or more ring atom selected from N, O, or S optionally substituted with 1-3 independently selected R^{A}.

11. The compound of any one of embodiments 1-8 and 10, wherein R² is a 5-10 membered heteroaryl containing one or more ring atom selected from N, O, or S substituted with 1-3 independently selected R^{A}.

12. The compound of embodiments 1-8 and 10-11, wherein the R² 5-10 membered heteroaryl containing one or more ring atom selected from N, O, or S is a 5-6 membered heteroaryl containing one or more ring atom selected from N, O, or S.

13. The compound of any one of embodiments 1-8 and 10-12, wherein the R² 5-10 membered heteroaryl containing one or more ring atom selected from N, O, or S is pyridyl, pyrazinyl, or pyrimidinyl.

14. The compound of any one of embodiments 1-8 and 10-13 wherein the R² 5-10 membered heteroaryl containing one or more ring atom selected from N, O, or S is pyridyl.

15. The compound of anyone of embodiments 1-8 and 10-14, wherein the R² pyridyl is 2-pyridyl.

16. The compound of embodiment 15, wherein R² is

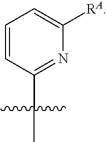

17. The compound of embodiment 16, wherein R² is selected from:

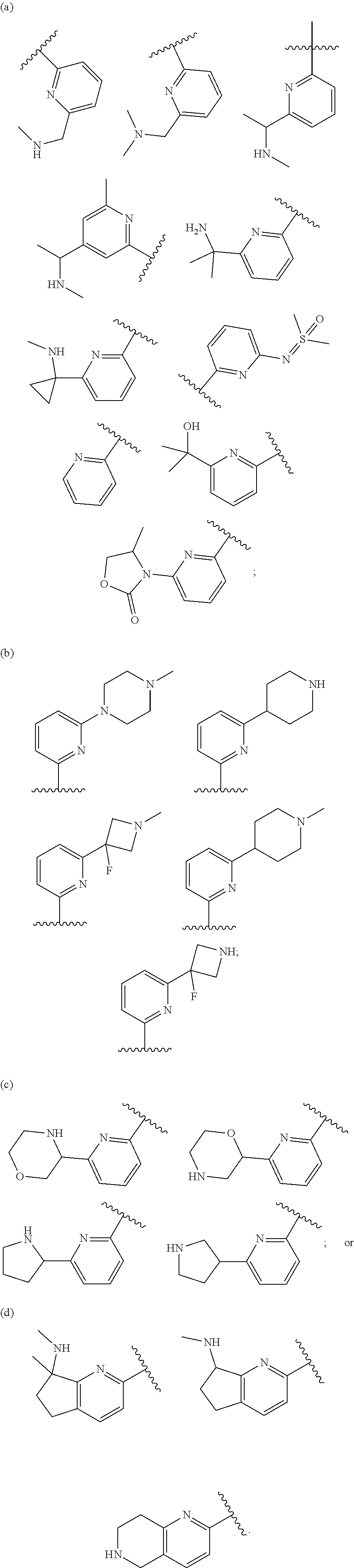

18. The compound of any one of embodiments 1-8, wherein R² is a 5-10 membered heterocyclyl containing one or more ring atom selected from N, O, S, C(O), N(O), S(O), or S(O)₂ optionally substituted with 1-3 independently selected R^{A}.

19. The compound of any one of embodiments 1-8 and 15, wherein R² is a 5-10 membered heterocyclyl containing one or more ring atom selected from N, O, S, C(O), N(O), S(O), or S(O)₂ substituted with 1-3 independently selected R^{A}.

20. The compound of any one of embodiments 1-16 and 18-19, wherein each R^{A} is independently selected from the group consisting of fluoro; chloro; hydroxyl; cyano; NR^{B}R^{C}; C(═O)NR^{B}R^{C}; C1-C6 alkyl, optionally substituted with hydroxyl, NR^{B}R^{C}, or C3-C6 cycloalkyl; C1-C6 alkoxy; C1-C6 haloalkyl, optionally substituted with NR^{B}R^{C}; C3-C6 cycloalkyl, optionally substituted with NR^{B}R^{C}; N═S(O)(Me)₂; N═S(O)(R^{J})2, where each R^{J} combines with the S atom to which it is attached to form a 5 membered ring; S(O)₂C1-C6 alkyl; S(O)(═NR^{B})C1-C6 alkyl; P(O)(C1-C6 alkyl)₂; and 4-6 membered heterocyclyl containing one or more ring atom selected from N, O, S, C(O), N(O), S(O), or S(O)₂ optionally substituted with halogen, hydroxyl, or C1-C6 alkyl.

21. The compound of any one of embodiments 1-16 and 18-20, wherein one or more R^{A} is an independently selected NR^{B}R^{C}.

22. The compound of any one of embodiments 1-16 and 18-21, wherein one or more R^{A} is an independently selected C(═O)NR^{B}R^{C}.

23. The compound of any one of embodiments 1-16 and 18-22, wherein R^{B} and R^{C} are each hydrogen.

24. The compound of any one of embodiments 1-16 and 18-22, wherein R^{B} is hydrogen and R^{C} is C1-C6 alkyl.

25. The compound of any one of embodiments 1-16 and 18-22, wherein R^{B} and R^{C} are each independently selected C1-C6 alkyl.

26. The compound of any one of embodiments 1-16 and 18-20, wherein one or more R^{A} is an independently selected C1-C6 alkyl optionally substituted with hydroxyl or NR^{B}R^{C}.

27. The compound of any one of embodiments 1-26, wherein R³ is hydrogen.

28. The compound of any one of embodiments 1-26, wherein R³ is C1-C6 alkyl.

29. The compound of any one of embodiments 1-26, wherein R³ is methyl.

30. The compound of any one of embodiments 1-29, wherein R⁴ is phenyl, optionally substituted with 1 or 2 substituents each independently selected from the group consisting of: halogen; cyano; SO₂(C1-C6 alkyl); C1-C6 haloalkyl; C1-C6 deuteroalkyl; C1-C6 alkyl, optionally substituted with 1 or 2 substituents each independently selected from: NR^{B}R^{C} and CO₂H; (C1-C6 alkyl)ₙ-C(═O)NR^{E}R^{F}; C3-C6 cycloalkyl optionally substituted with C1-C6 alkyl; 5-6 membered heteroaryl containing one or more ring atom selected from N, O, or S optionally substituted with C1-C6 alkyl; 4-12 membered heterocyclyloxy containing one or more ring atom selected from N, O, S, C(O), N(O), S(O), or S(O)₂, optionally substituted with 1 or 2 independently selected R^{G}; and 4-12 membered heterocyclyl containing one or more ring atom selected from N, O, S, C(O), N(O), S(O), or S(O)₂, optionally substituted with 1 or 2 independently selected R^{G}.

31. The compound of any one of embodiments 1-30, wherein R⁴ is phenyl substituted with 1 or 2 substituents each independently selected from the group consisting of: halogen; cyano; SO₂(C1-C6 alkyl); C1-C6 haloalkyl; C1-C6 deuteroalkyl; C1-C6 alkyl, optionally substituted with 1 or 2 substituents each independently selected from: NR^{B}R^{C} and CO₂H; (C1-C6 alkyl)ₙ-C(═O)NR^{E}R^{F}; C3-C6 cycloalkyl, optionally substituted with C1-C6 alkyl; 5-6 membered heteroaryl containing one or more ring atom selected from N, O, or S, optionally substituted with C1-C6 alkyl; 4-12 membered heterocyclyloxy containing one or more ring atom selected from N, O, S, C(O), N(O), S(O), or S(O)₂, optionally substituted with 1 or 2 independently selected R^{G}; and 4-12 membered heterocyclyl containing one or more ring atom selected from N, O, S, C(O), N(O), S(O), or S(O)₂, optionally substituted with 1 or 2 independently selected R^{G}.

32. The compound of any one of embodiments 1-31, wherein R⁴ is phenyl substituted with 1 or 2 substituents independently selected from the group consisting of halogen; C1-C6 alkyl; 5-6 membered heteroaryl containing one or more ring atom selected from N, O, or S optionally substituted with C1-C6 alkyl; 4-12 membered heterocyclyloxy containing one or more ring atom selected from N, O, S, C(O), N(O), S(O), or S(O)₂, optionally substituted with 1 or 2 independently selected R^{G}; and 4-12 membered heterocyclyl containing one or more ring atom selected from N, O, S, C(O), N(O), S(O), or S(O)₂, optionally substituted with 1 or 2 independently selected R^{G}.

33. The compound of embodiment 32, wherein R⁴ is phenyl substituted with 1 or 2 substituents independently selected from the group consisting of fluoro; chloro; C1-C3 alkyl; 5-6 membered heteroaryl containing one or more ring atom selected from N, O, or S, optionally substituted with C1-C6 alkyl; 4-12 membered heterocyclyloxy containing one or more ring atom selected from N, O, S, C(O), N(O), S(O), or S(O)₂, optionally substituted with 1 or 2 independently selected R^{G}; and 4-12 membered heterocyclyl containing one or more ring atom selected from N, O, S, C(O), N(O), S(O), or S(O)₂, substituted with 1 or 2 independently selected R^{G}.

34. The compound of embodiment 33, wherein R⁴ is phenyl is substituted with imidazolyl or pyrazolyl, each optionally substituted with 1 or 2 independently selected R^{G}.

35. The compound of embodiment 32, wherein R⁴ is phenyl is substituted with piperidinyl, piperazinyl, morpholinyl, pyrrolidinyl, pyrrolidinonyl, tetrahydrofuranyl, tetrahydropyranyl, oxetanyl, azetidinyl, 1,4-oxazepan-4-yl, 3-oxa-8-azabicyclo[3.2.1]octanyl, 2,5-diazabicyclo[2.2.1]heptanyl, 2-oxa-5-azabicyclo[2.2.1]heptanyl, 1,4-diazepanyl, 2-oxopiperazinyl, 3-oxopiperazinyl, thiomorpholinyl, thiomorpholinyl-1-oxide, octahydropyrrolo[3,4-c]pyrrolyl, or 2,6-diazaspiro[3.3]heptanyl, wherein each of which is optionally substituted with 1 or 2 independently selected R^{G}.

36. The compound of any one of embodiments 1-35, wherein each R^{G} is independently selected from halogen, C1-C6 alkyl, C1-C6 deuteroalkyl, and NR^{B}R^{C}.

37. The compound of any one of embodiments 1-36, wherein each R^{G} is independently selected from fluoro, chloro, methyl, CD₃, and NCH₃R^{C}, wherein R^{C} is selected from hydrogen and methyl.

38. The compound of any one of embodiments 1-29, wherein R⁴ is a 9-12 membered heterocyclyl containing one or more ring atom selected from N, O, S, C(O), N(O), S(O), or S(O)₂, optionally substituted with 1-3 independently selected C1-C6 alkyl.

39. The compound of any one of embodiments 1-29 and 38, wherein R⁴ is a 9-12 membered heterocyclyl containing one or more ring atom selected from N, O, S, C(O), N(O), S(O), or S(O)₂, substituted with 1 or 2 independently selected C1-C6 alkyl.

40. The compound of any one of embodiments 1-29 and 38, wherein R⁴ is an unsubstituted 9-12 membered heterocyclyl containing one or more ring atom selected from N, O, S, C(O), N(O), S(O), or S(O)₂.

41. The compound of any one of embodiments 1-29 and 38-40, wherein R⁴ is selected from

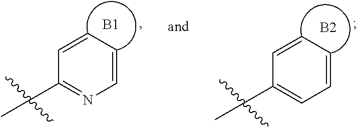

wherein each of Ring B1 and Ring B2 is independently selected 5-6 membered heterocyclyl containing one or more ring atom selected from N, O, S, C(O), N(O), S(O), or S(O)₂.

42. The compound of any one of embodiments 1-29, wherein R⁴ is a 5-6 membered heteroaryl containing one or more ring atom selected from N, O, or S, optionally substituted with 1 or 2 substituents independently selected from the group consisting of C1-C6 alkyl; 5-6 membered heteroaryl containing one or more ring atom selected from N, O, or S, optionally substituted with C1-C6 alkyl; 4-12 membered heterocyclyloxy containing one or more ring atom selected from N, O, S, C(O), N(O), S(O), or S(O)₂, optionally substituted with 1 or 2 independently selected R^{G}; and 4-12 membered heterocyclyl containing one or more ring atom selected from N, O, S, C(O), N(O), S(O), or S(O)₂, optionally substituted with C1-C6 alkyl or amino.

43. The compound of any one of embodiments 1-29 and 42, wherein R⁴ is a 5-6 membered heteroaryl containing one or more ring atom selected from N, O, or S, substituted with 1 or 2 substituents independently selected from the group consisting of C1-C6 alkyl; 5-6 membered heteroaryl containing one or more ring atom selected from N, O, or S, optionally substituted with C1-C6 alkyl; 4-12 membered heterocyclyloxy containing one or more ring atom selected from N, O, S, C(O), N(O), S(O), or S(O)₂, optionally substituted with 1 or 2 independently selected R^{G}; and 4-12 membered heterocyclyl containing one or more ring atom selected from N, O, S, C(O), N(O), S(O), or S(O)₂, optionally substituted with C1-C6 alkyl or amino.

44. The compound of any one of embodiments 1-29, 42 and 43, wherein R⁴ is a 5-6 membered heteroaryl containing one or more ring atom selected from N, O, or S, substituted with 1 or 2 independently selected 4-12 membered heterocyclyl groups containing one or more ring atom selected from N, O, S, C(O), N(O), S(O), or S(O) 2, each optionally substituted with C1-C6 alkyl or amino.

45. The compound of any one of embodiments 1-29 and 42-44, wherein R⁴ is a 5-6 membered heteroaryl containing one or more ring atom selected from N, O, or S, substituted with 1 or 2 substituents independently selected from the group consisting of piperidinyl, piperazinyl, and tetrahydropyranyl, each optionally substituted with methyl or amino.

46. The compound of any one of embodiments 1-29, 42, and 43, wherein R⁴ is a 5-6 membered heteroaryl containing one or more ring atom selected from N, O, or S, substituted with 1 or 2 independently selected C1-C6 alkyl.

47. The compound of any one of embodiments 1-29, 42, and 43, wherein R⁴ is a 5-6 membered heteroaryl containing one or more ring atom selected from N, O, or S, substituted with 1 or 2 substituents independently selected from 4-12 membered heterocyclyloxy containing one or more ring atom selected from N, O, S, C(O), N(O), S(O), or S(O)₂, optionally substituted with 1 or 2 independently selected R^{G}.

48. The compound of any one of embodiments 1-29, 42, 43, and 46, wherein R⁴ is a 5-6 membered heteroaryl containing one or more ring atom selected from N, O, or S, substituted with 1 or 2 substituents independently selected from the group consisting of azetidinyloxy, piperidinyloxy, and pyrrolidinyloxy, each optionally substituted with 1 or 2 independently selected R^{G}.

49. The compound of any one of embodiments 1-29, 42, and 43, wherein the R⁴ is a 5-6 membered heteroaryl containing one or more ring atom selected from N, O, or S, selected from the group consisting of pyrazol-4-yl, pyrazol-3-yl, imidazolyl, isoxazolyl, thiazolyl, 1,2,3-triazol-4-yl, pyridin-3-yl, pyazinyl, pyrimidinyl, and pyridazinyl.

50. The compound of any one of embodiments 1-29, wherein R⁴ is C3-C6 cycloalkyl.

51. The compound according to any one of embodiments 1-29, wherein R⁴ is C(O)—R^{I}.

52. The compound of Embodiment 1 or Embodiment 2, wherein the compound is selected from the group consisting of the compounds in Tables 1 and 2, or a pharmaceutically acceptable salt thereof.

53. A pharmaceutical composition comprising a compound of any one of Embodiments 1-52, or a pharmaceutically acceptable salt thereof, and at least one pharmaceutically acceptable excipient.

54. A method for treating cancer in a subject in need thereof, comprising administering to the subject an effective amount of a compound of any one of Embodiments 1-52 or a pharmaceutically acceptable salt thereof, or a pharmaceutical composition according to Embodiment 53.

55. The method of embodiment 54, wherein the cancer is selected from one or more of uterine, ovarian, breast, gastric, colorectal, and non-small cell lung.

56. Use of a compound of any one of embodiments 1-52 or a composition of embodiment 53 in the manufacture of a medicament for treating cancer in a patient in need thereof.

57. The use of embodiment 56, wherein the cancer is selected from one or more of uterine, ovarian, breast, gastric, colorectal, and non-small cell lung.

58. A compound or composition for use in the treatment of cancer comprising a compound of any one of embodiments 1-52, or a composition of embodiment 53.

59. The compound or composition of embodiment 58, wherein the cancer is selected from one or more of uterine, ovarian, breast, gastric, colorectal, and non-small cell lung.

A number of embodiments of the present disclosure have been described. Nevertheless, it will be understood that various modifications may be made without departing from the spirit and scope of the disclosure. Accordingly, other embodiments are within the scope of the following claims.

What is claimed is:

1. A compound of Formula (I): 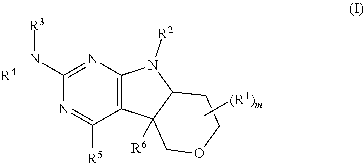
or a pharmaceutically acceptable salt thereof, wherein:
each R¹ is independently C1-C6 alkyl;
m is 0, 1, or 2;
R² is hydrogen, C1-C6 alkyl, phenyl, 5-10 membered heteroaryl containing one or more ring atom selected from N, O, or S, or 5-10 membered heterocyclyl containing one or more ring atom selected from N, O, S, C(O), N(O), S(O), or S(O)₂, wherein each of phenyl, 5-10 membered heteroaryl, and 5-10 membered heterocyclyl is optionally substituted with 1-3 substituents each independently selected from R^{A};
each R^{A} is independently selected from: halogen; C1-C6 haloalkyl, optionally substituted with NR^{B}R^{C}; hydroxyl; cyano; NR^{B}R^{C}; C(═O)NR^{B}R^{C}; N═S(O)(R^{J})₂, wherein each R^{J} is methyl or both taken together with the S atom to which they are attached form a 5 membered ring; S(O)₂C1-C6 alkyl; S(O)(═NR^{B})C1-C6 alkyl; P(O)(C1-C6 alkyl)₂; C1-C6 alkyl, optionally substituted with hydroxyl, or NR^{B}R^{C}; C1-C6 alkoxy; C3-C6 cycloalkyl, optionally substituted with hydroxyl or NR^{B}R^{C}; and 4-6 membered heterocyclyl containing one or more ring atom selected from N, O, S, C(O), N(O), S(O), or S(O)², optionally substituted with 1-2 substituents independently selected from halogen, hydroxyl, and C1-C6 alkyl;
each R^{B} and R^{C} are independently hydrogen or C1-C6 alkyl;
R³ is hydrogen or C1-C6 alkyl;
R⁴ is
(iii) hydrogen;
(iv) phenyl, optionally substituted with 1 or 2 substituents each independently selected from the group consisting of:
halogen; cyano; SO₂(C1-C6 alkyl); C1-C6 haloalkyl; C1-C6 deuteroalkyl; C1-C6 alkyl, optionally substituted with 1 or 2 substituents each independently selected from: —NR^{B}R^{C} and —CO₂H; (C1-C6 alkyl)ₙ-C(═O)NR^{E}R^{F}; C3-C6 cycloalkyl, optionally substituted with C1-C6 alkyl; 5-6 membered heteroaryl containing one or more ring atom selected from N, O, or S, optionally substituted with C1-C6 alkyl; 4-12 membered heterocyclyloxy containing one or more ring atom selected from N, O, S, C(O), N(O), S(O), or S(O)₂, optionally substituted with 1 or 2 independently selected R^{G}; and 4-12 membered heterocyclyl containing one or more ring atom selected from N, O, S, C(O), N(O), S(O), or S(O)₂, optionally substituted with 1, 2, or 3 independently selected R^{G};
(iii) 9-12 membered heterocyclyl containing one or more ring atom selected from N, O, S, C(O), N(O), S(O), or S(O)₂, optionally substituted with 1-4 independently selected C1-C6 alkyl, or halogen;
(iv) 5-10 membered heteroaryl containing one or more ring atom selected from N, O, or S, optionally substituted with 1 or 2 substituents independently selected from the group consisting of C1-C6 alkyl; cyano; halogen; C1-C6 haloalkyl; C1-C6 deuteroalkyl; C1-C6 alkoxy; C3-C6 cycloalkyl; 5-6 membered heteroaryl containing one or more ring atom selected from N, O, or S, optionally substituted with C1-C6 alkyl; 4-12 membered heterocyclyloxy containing one or more ring atom selected from N, O, S, C(O), N(O), S(O), or S(O)₂, optionally substituted with 1 or 2 independently selected R^{G}; and 4-12 membered heterocyclyl containing one or more ring atom selected from N, O, S, C(O), N(O), S(O), or S(O)₂, optionally substituted with 1 or 2 C1-C6 alkyl, C1-C6 alkoxy, amino, or 4-12 membered heterocyclyl containing one or more ring atom selected from N, O, S, C(O), N(O), S(O), or S(O)₂;
(v) C3-C6 cycloalkyl; or
(vi) C(O)—R^{I};
n is 0 or 1;
each R^{E} and R^{F} are independently hydrogen or C1-C6 alkyl; or
R^{E} and R^{F}, together with the nitrogen atom to which they are attached, form a 4-8 membered heterocyclyl containing one or more ring atom selected from N, O, S, C(O), N(O), S(O), or S(O)₂, optionally substituted with C1-C6 alkyl;
each R^{G} is independently halogen, cyano; C1-C6 alkyl, C1-C6 deuteroalkyl, NR^{B}R^{C}, or ═NR^{H};
R^{H} is hydrogen or C1-C6 alkyl;
R^{I} is selected from the group consisting of C1-C6 alkyl; phenyl, optionally substituted with 1 to 3 halogen; 5-6 membered heteroaryl containing one or more ring atom selected from N, O, or S, optionally substituted with 1 to 3 C1-C6 alkyl; C3-C6 cycloalkyl, optionally substituted with 1 substituent selected from the group consisting of: halogen, phenyl, 5-6 membered heterocyclyl containing one or more ring atom selected from N, O, S, C(O), N(O), S(O), or S(O)₂ and optionally substituted with 1 to 3 C1-C6 alkyl, and 5-6 membered heteroaryl containing one or more ring atom selected from N, O, or S, optionally substituted with 1 to 3 C1-C6 alkyl; and 4-12 membered heterocyclyloxy containing one or more ring atom selected from N, O, S, C(O), N(O), S(O), or S(O)₂, optionally substituted with 1 or 2 C1-C6 alkyl;
R⁵ is hydrogen, halogen, or C1-C6 alkyl; and
R⁶ is hydrogen or C1-C6 alkyl.

2. A compound of Formula (I): 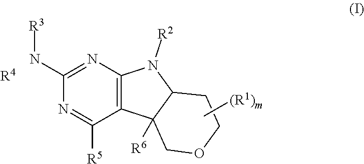
or a pharmaceutically acceptable salt thereof, wherein:
each R¹ is independently C1-C6 alkyl;
m is 0, 1, or 2;
R² is hydrogen, C1-C6 alkyl, phenyl, 5-10 membered heteroaryl containing one or more ring atom selected from N, O, or S, or 5-10 membered heterocyclyl containing one or more ring atom selected from N, O, S, C(O), N(O), S(O), or S(O)₂, wherein the phenyl, 5-10 membered heteroaryl, and 5-10 membered heterocyclyl are each optionally substituted with 1-3 substituents each independently selected from R^{A};
each R^{A} is independently selected from: halogen, cyano, —NR^{B}R^{C}, —C(═O)NR^{B}R^{C}, —N═S(O)Me)₂, C1-C6 alkyl optionally substituted with hydroxyl or —NR^{B}R^{C}; C3-C6 cycloalkyl optionally substituted with —NR^{B}R^{C}; and 4-6 membered heterocyclyl containing one or more ring atom selected from N, O, S, C(O), N(O), S(O), or S(O)₂ optionally substituted with 1-2 substituents independently selected from halogen and C1-C6 alkyl;
each R^{B} and R^{C} are independently hydrogen or C1-C6 alkyl;
R³ is hydrogen or C1-C6 alkyl;
R⁴ is
(i) phenyl optionally substituted with 1 or 2 substituents each independently selected from the group consisting of: halogen, cyano, —SO₂(C1-C6 alkyl), C1-C6 haloalkyl, C1-C6 deuteroalkyl, C1-C6 alkyl optionally substituted with 1 or 2 substituents each independently selected from: —NR^{B}R^{C} and —CO₂H; —(C1-C6 alkyl)ₙ-C(═O)NR^{E}R^{F}, C3-C6 cycloalkyl optionally substituted with C1-C6 alkyl; 5-6 membered heteroaryl containing one or more ring atom selected from N, O, or S optionally substituted with C1-C6 alkyl; 4-12 membered heterocyclyloxy containing one or more ring atom selected from N, O, S, C(O), N(O), S(O), or S(O)₂ optionally substituted with 1 or 2 independently selected R^{G}; and 4-12 membered heterocyclyl containing one or more ring atom selected from N, O, S, C(O), N(O), S(O), or S(O)₂ optionally substituted with 1 or 2 independently selected R^{G};
(ii) 9-12 membered heterocyclyl containing one or more ring atom selected from N, O, S, C(O), N(O), S(O), or S(O)₂ optionally substituted with 1-3 independently selected C1-C6 alkyl;
(iii) 5-10 membered heteroaryl containing one or more ring atom selected from N, O, or S optionally substituted with 1 or 2 substituents independently selected from the group consisting of C1-C6 alkyl, C1-C6 haloalkyl, C1-C6 deuteroalkyl, C3-C6 cycloalkyl, 5-6 membered heteroaryl containing one or more ring atom selected from N, O, or S optionally substituted with C1-C6 alkyl; 4-12 membered heterocyclyloxy containing one or more ring atom selected from N, O, S, C(O), N(O), S(O), or S(O)₂ optionally substituted with 1 or 2 independently selected R^{G}; and 4-12 membered heterocyclyl containing one or more ring atom selected from N, O, S, C(O), N(O), S(O), or S(O)₂ optionally substituted with 1 or 2 C1-C6 alkyl or amino;
(iv) C3-C6 cycloalkyl; or
(v) C(O)—R^{I};
n is 0 or 1;
each R^{E} and R^{F} are independently hydrogen or C1-C6 alkyl; or
R^{E} and R^{F}, together with the nitrogen atom to which they are attached, form a 4-8 membered heterocyclyl containing one or more ring atom selected from N, O, S, C(O), N(O), S(O), or S(O)₂ optionally substituted with C1-C6 alkyl;
each R^{G} is independently halogen, C1-C6 alkyl, C1-C6 deuteroalkyl, —NR^{B}R^{C}, or ═NR^{H};
R^{H} is hydrogen or C1-C6 alkyl;
R^{I} is C1-C6 alkyl, phenyl optionally substituted with 1 to 3 halogen; 5-6 membered heteroaryl containing one or more ring atom selected from N, O, or S optionally substituted with 1 to 3 C1-C6 alkyl; C3-C6 cycloalkyl optionally substituted with 1 substituent selected from the group consisting of: halogen, phenyl, and 5-6 membered heteroaryl containing one or more ring atom selected from N, O, or S optionally substituted with 1 to 3 C1-C6 alkyl;
R⁵ is hydrogen, halogen, or C1-C6 alkyl; and
R⁶ is hydrogen or C1-C6 alkyl.

3. The compound of claim 1 or 2, wherein m is 2.

4. The compound of any one of claims 1 to 3, wherein each R¹ is methyl.

5. The compound of any one of claims 1-4, wherein two independently selected R¹ groups are geminal, and the carbon atom to which they are attached is adjacent to the ring oxygen.

6. The compound according to any one of claims 1-5, wherein R⁵ is hydrogen.

7. The compound according to any one of claims 1-6, wherein R⁶ is methyl.

8. The compound according to any one of claims 1-7 having the following structure:

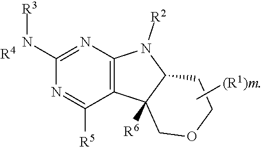

9. The compound of any one of claims 1-8, wherein R² is phenyl optionally substituted with 1-3 independently selected R^{A}.

10. The compound of any one of claims 1-9, wherein R² is a 5-10 membered heteroaryl containing one or more ring atom selected from N, O, or S optionally substituted with 1-3 independently selected R^{A}.

11. The compound of any one of claims 1-8 and 10, wherein R² is a 5-10 membered heteroaryl containing one or more ring atom selected from N, O, or S substituted with 1-3 independently selected R^{A}.

12. The compound of claims 1-8 and 10-11, wherein the R² 5-10 membered heteroaryl containing one or more ring atom selected from N, O, or S is a 5-6 membered heteroaryl containing one or more ring atom selected from N, O, or S.

13. The compound of any one of claims 1-8 and 10-12, wherein the R² 5-10 membered heteroaryl containing one or more ring atom selected from N, O, or S is pyridyl, pyrazinyl, or pyrimidinyl.

14. The compound of any one of claims 1-8 and 10-13 wherein the R² 5-10 membered heteroaryl containing one or more ring atom selected from N, O, or S is pyridyl.

15. The compound of anyone of claims 1-8 and 10-14, wherein the R² pyridyl is 2-pyridyl.

16. The compound of claim 15, wherein R² is

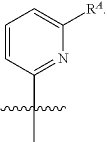

17. The compound of claim 16, wherein R² is selected from:

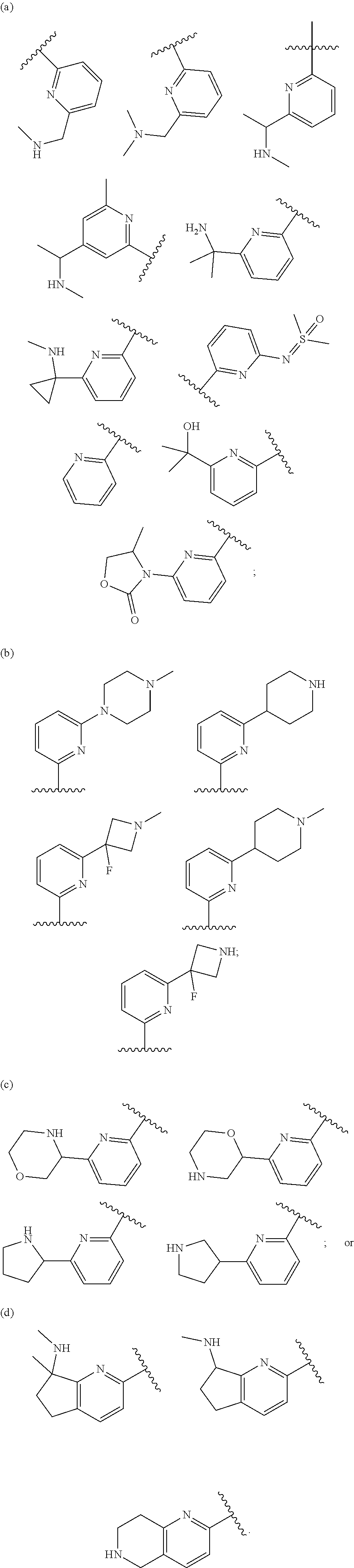

18. The compound of any one of claims 1-8, wherein R² is a 5-10 membered heterocyclyl containing one or more ring atom selected from N, O, S, C(O), N(O), S(O), or S(O)₂ optionally substituted with 1-3 independently selected R^{A}.

19. The compound of any one of claims 1-8 and 15, wherein R² is a 5-10 membered heterocyclyl containing one or more ring atom selected from N, O, S, C(O), N(O), S(O), or S(O)₂ substituted with 1-3 independently selected R^{A}.

20. The compound of any one of claims 1-16 and 18-19, wherein each R^{A} is independently selected from the group consisting of fluoro; chloro; hydroxyl; cyano; NR^{B}R^{C}; C(═O)NR^{B}R^{C}; C1-C6 alkyl, optionally substituted with hydroxyl, NR^{B}R^{C}, or C3-C6 cycloalkyl; C1-C6 alkoxy; C1-C6 haloalkyl, optionally substituted with NR^{B}R^{C}; C3-C6 cycloalkyl, optionally substituted with NR^{B}R^{C}; N═S(O)(Me)₂; N═S(O)(R^{J})₂, where each R^{J} combines with the S atom to which it is attached to form a 5 membered ring; S(O)₂C1-C6 alkyl; S(O)(═NR^{B})C1-C6 alkyl; P(O)(C1-C6 alkyl)₂; and 4-6 membered heterocyclyl containing one or more ring atom selected from N, O, S, C(O), N(O), S(O), or S(O)₂ optionally substituted with halogen, hydroxyl, or C1-C6 alkyl.

21. The compound of any one of claims 1-16 and 18-20, wherein one or more R^{A} is an independently selected NR^{B}R^{C}.

22. The compound of any one of claims 1-16 and 18-21, wherein one or more R^{A} is an independently selected C(═O)NR^{B}R^{C}.

23. The compound of any one of claims 1-16 and 18-22, wherein R^{B} and R^{C} are each hydrogen.

24. The compound of any one of claims 1-16 and 18-22, wherein R^{B} is hydrogen and R^{C} is C1-C6 alkyl.

25. The compound of any one of claims 1-16 and 18-22, wherein R^{B} and R^{C} are each independently selected C1-C6 alkyl.

26. The compound of any one of claims 1-16 and 18-20, wherein one or more R^{A} is an independently selected C1-C6 alkyl optionally substituted with hydroxyl or NR^{B}R^{C}.

27. The compound of any one of claims 1-26, wherein R³ is hydrogen.

28. The compound of any one of claims 1-26, wherein R³ is C1-C6 alkyl.

29. The compound of any one of claims 1-26, wherein R³ is methyl.

30. The compound of any one of claims 1-29, wherein R⁴ is phenyl, optionally substituted with 1 or 2 substituents each independently selected from the group consisting of: halogen; cyano; SO₂(C1-C6 alkyl); C1-C6 haloalkyl; C1-C6 deuteroalkyl; C1-C6 alkyl, optionally substituted with 1 or 2 substituents each independently selected from: NR^{B}R^{C} and CO₂H; (C1-C6 alkyl)ₙ-C(═O)NR^{E}R^{F}; C3-C6 cycloalkyl optionally substituted with C1-C6 alkyl; 5-6 membered heteroaryl containing one or more ring atom selected from N, O, or S optionally substituted with C1-C6 alkyl; 4-12 membered heterocyclyloxy containing one or more ring atom selected from N, O, S, C(O), N(O), S(O), or S(O)₂, optionally substituted with 1 or 2 independently selected R^{G}; and 4-12 membered heterocyclyl containing one or more ring atom selected from N, O, S, C(O), N(O), S(O), or S(O)₂, optionally substituted with 1 or 2 independently selected R^{G}.

31. The compound of any one of claims 1-30, wherein R⁴ is phenyl substituted with 1 or 2 substituents each independently selected from the group consisting of: halogen; cyano; SO₂(C1-C6 alkyl); C1-C6 haloalkyl; C1-C6 deuteroalkyl; C1-C6 alkyl, optionally substituted with 1 or 2 substituents each independently selected from: NR^{B}R^{C} and CO₂H; (C1-C6 alkyl)ₙ-C(═O)NR^{E}R^{F}; C3-C6 cycloalkyl, optionally substituted with C1-C6 alkyl; 5-6 membered heteroaryl containing one or more ring atom selected from N, O, or S, optionally substituted with C1-C6 alkyl; 4-12 membered heterocyclyloxy containing one or more ring atom selected from N, O, S, C(O), N(O), S(O), or S(O)₂, optionally substituted with 1 or 2 independently selected R^{G}; and 4-12 membered heterocyclyl containing one or more ring atom selected from N, O, S, C(O), N(O), S(O), or S(O)₂, optionally substituted with 1 or 2 independently selected R^{G}.

32. The compound of any one of claims 1-31, wherein R⁴ is phenyl substituted with 1 or 2 substituents independently selected from the group consisting of halogen; C1-C6 alkyl; 5-6 membered heteroaryl containing one or more ring atom selected from N, O, or S optionally substituted with C1-C6 alkyl; 4-12 membered heterocyclyloxy containing one or more ring atom selected from N, O, S, C(O), N(O), S(O), or S(O)₂, optionally substituted with 1 or 2 independently selected R^{G}; and 4-12 membered heterocyclyl containing one or more ring atom selected from N, O, S, C(O), N(O), S(O), or S(O)₂, optionally substituted with 1 or 2 independently selected R^{G}.

33. The compound of claim 32, wherein R⁴ is phenyl substituted with 1 or 2 substituents independently selected from the group consisting of fluoro; chloro; C1-C3 alkyl; 5-6 membered heteroaryl containing one or more ring atom selected from N, O, or S, optionally substituted with C1-C6 alkyl; 4-12 membered heterocyclyloxy containing one or more ring atom selected from N, O, S, C(O), N(O), S(O), or S(O)₂, optionally substituted with 1 or 2 independently selected R^{G}; and 4-12 membered heterocyclyl containing one or more ring atom selected from N, O, S, C(O), N(O), S(O), or S(O)₂, substituted with 1 or 2 independently selected R^{G}.

34. The compound of claim 33, wherein R⁴ is phenyl is substituted with imidazolyl or pyrazolyl, each optionally substituted with 1 or 2 independently selected R^{G}.

35. The compound of claim 32, wherein R⁴ is phenyl is substituted with piperidinyl, piperazinyl, morpholinyl, pyrrolidinyl, pyrrolidinonyl, tetrahydrofuranyl, tetrahydropyranyl, oxetanyl, azetidinyl, 1,4-oxazepan-4-yl, 3-oxa-8-azabicyclo[3.2.1]octanyl, 2,5-diazabicyclo[2.2.1]heptanyl, 2-oxa-5-azabicyclo[2.2.1]heptanyl, 1,4-diazepanyl, 2-oxopiperazinyl, 3-oxopiperazinyl, thiomorpholinyl, thiomorpholinyl-1-oxide, octahydropyrrolo[3,4-c]pyrrolyl, or 2,6-diazaspiro[3.3]heptanyl, wherein each of which is optionally substituted with 1 or 2 independently selected R^{G}.

36. The compound of any one of claims 1-35, wherein each R^{G} is independently selected from halogen, C1-C6 alkyl, C1-C6 deuteroalkyl, and NR^{B}R^{C}.

37. The compound of any one of claims 1-36, wherein each R^{G} is independently selected from fluoro, chloro, methyl, CD₃, and NCH₃R^{C}, wherein R^{C} is selected from hydrogen and methyl.

38. The compound of any one of claims 1-29, wherein R⁴ is a 9-12 membered heterocyclyl containing one or more ring atom selected from N, O, S, C(O), N(O), S(O), or S(O)₂, optionally substituted with 1-3 independently selected C1-C6 alkyl.

39. The compound of any one of claims 1-29 and 38, wherein R⁴ is a 9-12 membered heterocyclyl containing one or more ring atom selected from N, O, S, C(O), N(O), S(O), or S(O)₂, substituted with 1 or 2 independently selected C1-C6 alkyl.

40. The compound of any one of claims 1-29 and 38, wherein R⁴ is an unsubstituted 9-12 membered heterocyclyl containing one or more ring atom selected from N, O, S, C(O), N(O), S(O), or S(O)₂.

41. The compound of any one of claims 1-29 and 38-40, wherein R⁴ is selected from 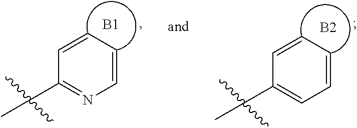
wherein each of Ring B1 and Ring B2 is independently selected 5-6 membered heterocyclyl containing one or more ring atom selected from N, O, S, C(O), N(O), S(O), or S(O)₂.

42. The compound of any one of claims 1-29, wherein R⁴ is a 5-6 membered heteroaryl containing one or more ring atom selected from N, O, or S, optionally substituted with 1 or 2 substituents independently selected from the group consisting of C1-C6 alkyl; 5-6 membered heteroaryl containing one or more ring atom selected from N, O, or S, optionally substituted with C1-C6 alkyl; 4-12 membered heterocyclyloxy containing one or more ring atom selected from N, O, S, C(O), N(O), S(O), or S(O)₂, optionally substituted with 1 or 2 independently selected R^{G}; and 4-12 membered heterocyclyl containing one or more ring atom selected from N, O, S, C(O), N(O), S(O), or S(O)₂, optionally substituted with C1-C6 alkyl or amino.

43. The compound of any one of claims 1-29 and 42, wherein R⁴ is a 5-6 membered heteroaryl containing one or more ring atom selected from N, O, or S, substituted with 1 or 2 substituents independently selected from the group consisting of C1-C6 alkyl; 5-6 membered heteroaryl containing one or more ring atom selected from N, O, or S, optionally substituted with C1-C6 alkyl; 4-12 membered heterocyclyloxy containing one or more ring atom selected from N, O, S, C(O), N(O), S(O), or S(O)₂, optionally substituted with 1 or 2 independently selected R^{G}; and 4-12 membered heterocyclyl containing one or more ring atom selected from N, O, S, C(O), N(O), S(O), or S(O)₂, optionally substituted with C1-C6 alkyl or amino.

44. The compound of any one of claims 1-29, 42 and 43, wherein R⁴ is a 5-6 membered heteroaryl containing one or more ring atom selected from N, O, or S, substituted with 1 or 2 independently selected 4-12 membered heterocyclyl groups containing one or more ring atom selected from N, O, S, C(O), N(O), S(O), or S(O)₂, each optionally substituted with C1-C6 alkyl or amino.

45. The compound of any one of claims 1-29 and 42-44, wherein R⁴ is a 5-6 membered heteroaryl containing one or more ring atom selected from N, O, or S, substituted with 1 or 2 substituents independently selected from the group consisting of piperidinyl, piperazinyl, and tetrahydropyranyl, each optionally substituted with methyl or amino.

46. The compound of any one of claims 1-29, 42, and 43, wherein R⁴ is a 5-6 membered heteroaryl containing one or more ring atom selected from N, O, or S, substituted with 1 or 2 independently selected C1-C6 alkyl.

47. The compound of any one of claims 1-29, 42, and 43, wherein R⁴ is a 5-6 membered heteroaryl containing one or more ring atom selected from N, O, or S, substituted with 1 or 2 substituents independently selected from 4-12 membered heterocyclyloxy containing one or more ring atom selected from N, O, S, C(O), N(O), S(O), or S(O)₂, optionally substituted with 1 or 2 independently selected R^{G}.

48. The compound of any one of claims 1-29, 42, 43, and 46, wherein R⁴ is a 5-6 membered heteroaryl containing one or more ring atom selected from N, O, or S, substituted with 1 or 2 substituents independently selected from the group consisting of azetidinyloxy, piperidinyloxy, and pyrrolidinyloxy, each optionally substituted with 1 or 2 independently selected RG.

49. The compound of any one of claims 1-29, 42, and 43, wherein the R⁴ is a 5-6 membered heteroaryl containing one or more ring atom selected from N, O, or S, selected from the group consisting of pyrazol-4-yl, pyrazol-3-yl, imidazolyl, isoxazolyl, thiazolyl, 1,2,3-triazol-4-yl, pyridin-3-yl, pyazinyl, pyrimidinyl, and pyridazinyl.

50. The compound of any one of claims 1-29, wherein R⁴ is C3-C6 cycloalkyl.

51. The compound according to any one of claims 1-29, wherein R⁴ is C(O)—R^{I}.

52. The compound of claim 1 or claim 2, wherein the compound is selected from the group consisting of the compounds in Tables 1 and 2, or a pharmaceutically acceptable salt thereof.

53. A pharmaceutical composition comprising a compound of any one of claims 1-52, or a pharmaceutically acceptable salt thereof, and at least one pharmaceutically acceptable excipient.

54. A method for treating cancer in a subject in need thereof, comprising administering to the subject an effective amount of a compound of any one of claims 1-52 or a pharmaceutically acceptable salt thereof, or a pharmaceutical composition according to claim 53.

55. The method of claim 54, wherein the cancer is selected from one or more of uterine, ovarian, breast, gastric, colorectal, and non-small cell lung.

56. Use of a compound of any one of claims 1-52 or a composition of claim 53 in the manufacture of a medicament for treating cancer in a patient in need thereof.

57. The use of claim 56, wherein the cancer is selected from one or more of uterine, ovarian, breast, gastric, colorectal, and non-small cell lung.

58. A compound or composition for use in the treatment of cancer comprising a compound of any one of claims 1-52, or a composition of claim 53.

59. The compound or composition of claim 58, wherein the cancer is selected from one or more of uterine, ovarian, breast, gastric, colorectal, and non-small cell lung.
